(19) United States
(12) Patent Application Publication
(10) Pub. No.: US 2023/0167133 A1
(43) Pub. Date: Jun. 1, 2023

(54) 7H-PYRROLO[2,3-D]PYRIMIDINES AND PREPARATION AND USES THEREOF

(71) Applicant: BioSplice Therapeutics, Inc., San Diego, CA (US)

(72) Inventors: Gopi Kumar Mittapalli, San Diego, CA (US); Sunil Kumar KC, San Diego, CA (US); Chi Ching Mak, San Diego, CA (US); Brian Joseph Hofilena, San Diego, CA (US); Chandramouli Chiruta, San Diego, CA (US); Ramkrishna Reddy Vakiti, San Diego, CA (US); Brian Walter Eastman, San Diego, CA (US); Joseph Timothy Marakovits, Encinitas, CA (US); Lewis Daniel Turner, San Diego, CA (US)

(21) Appl. No.: 17/964,470

(22) Filed: Oct. 12, 2022

Related U.S. Application Data
(60) Provisional application No. 63/330,480, filed on Apr. 13, 2022.
(60) Provisional application No. 63/254,708, filed on Oct. 12, 2021.

(51) Int. Cl. C07D 519/00; C07D 487/04

(52) U.S. Cl. CPC C07D 519/00; C07D 487/04; C07B 2200/05

(57) ABSTRACT

7H-Pyrrolo[2,3-d]pyrimidine compound for treating various diseases and pathologies are disclosed. More particularly, the present disclosure concerns the use of 7H-pyrrolo[2,3-d]pyrimidine compounds or analogs thereof, in the treatment of disorders characterized by overexpression of DYRK1A (e.g., cancer, Down syndrome, Alzheimer's disease, diabetes, viral infections, and osteoarthritis).

### RELATED APPLICATIONS

This application claims the benefit of U.S. Provisional Application Nos. 63/254,708, filed Oct. 12, 2021, and 63/330,480, filed Apr. 13, 2022, which are incorporated herein by reference in its entirety.

### BACKGROUND

### Technical Field

This disclosure relates to inhibitors of dual-specificity tyrosine phosphorylation-regulated lA kinase, and compositions comprising the same. More particularly, it concerns the use of a 7H-pyrrolo[2,3-d]pyrimidine compound or salts or analogs thereof, in the treatment of disorders characterized by the abnormal expression and/or activity of DYRK1A (e.g., cancer, Down syndrome, Alzheimer's disease, diabetes, viral infections, and osteoarthritis).

### Background

Dual-specificity tyrosine phosphorylation-regulated kinases (DYRK1A, 1B, 2-4) comprise a family of protein kinases within the CMGC group of the eukaryotic kinome. These protein kinases are involved in multiple cellular functions, including intracellular signaling, mRNA splicing, chromatin transcription, DNA damage repair, cell survival, cell cycle control, differentiation, homocysteine/methionine/folate regulation, body temperature regulation, endocytosis, neuronal development, synaptic plasticity, etc. Abnormal expression and/or activity of some of these kinases, DYRK1A in particular, is seen in many human nervous system diseases, such as cognitive deficits associated with Down syndrome, Alzheimer's disease, and related diseases, tauopathies, dementia, Pick's disease, Parkinson's disease, and other neurodegenerative diseases, Phelan-McDermid syndrome, autism, and CDKLS deficiency disorder. DYRKs are also involved in diabetes, abnormal folate/methionine metabolism, osteoarthritis, several solid cancers (glioblastoma, breast, and pancreatic cancers) and leukemias (acute lymphoblastic leukemia, acute megakaryoblastic leukemia), viral infections (influenza, HIV-1, HCMV, HCV, CMV, HPV), as well as infections caused by unicellular parasites (Leishmania, Trypanosoma, Plasmodium) (International Journal of Molecular Sciences (2021), 22(11), 6047). DYRK1A has also been identified as a critical stabilizer of EGFR (Cell Death & Disease (2019), 10, 282) which is a crucial factor contributing to the keratinization, cell hyperproliferation, abnormal differentiation and inflammatory infiltration during the progress of psoriasis.

### SUMMARY

The present disclosure provides methods and reagents, involving contacting a cell with an agent, such as a 7H-pyrrolo[2,3-d]pyrimidine compound, in a sufficient amount to antagonize DYRK1A activity, e.g., reduced the proliferation of head and neck squamous cell carcinoma, luminal/HER2 breast cancer (Cell (2016), 164(1-2), 293-309) or pancreatic adenocarcinoma, as well as impaired the self-renewal capacity of glioblastoma and compromised ovarian cancer spheroid cell viability (Molecular Cancer Research (2017), 15(4), 371-381).

The present disclosure also provides methods and reagents, involving contacting a cell with an agent, such as a 7H-pyrrolo[2,3-d]pyrimidine compound, in a sufficient amount to antagonize DYRK1A activity, e.g., i) to normalize prenatal and early postnatal brain development; ii) to improve cognitive function in youth and adulthood; and/or iii) to attenuate Alzheimer's-type neurodegeneration.

Some embodiments disclosed herein include DYRK1A inhibitors containing a 7H-pyrrolo[2,3-d]pyrimidine core. Other embodiments disclosed herein include pharmaceutical compositions and methods of treatment using these compounds.

One embodiment disclosed herein includes a compound having the structure of Formula I:

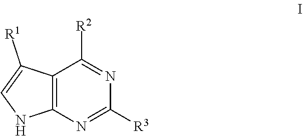

or a pharmaceutically acceptable salt thereof,

wherein:

R¹ is 8-11-membered bicyclic heteroaryl optionally substituted with 1-10 R⁴; wherein a carbon atom on an aromatic ring of the heteroaryl forms the bond with

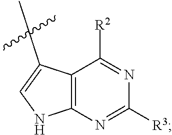

R² is selected from the group consisting of H, —OR⁷, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(Cl₁₋₉alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, -carbocyclyl optionally substituted with 1-12 R¹⁸, —C(═O)N(R¹⁹)₂, and —C(═O)R²⁰;

alternatively, two R⁴ attached to the same carbon atom are taken together to form a carbonyl group;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₃ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸;

each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁵;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁵;

each R²⁶ is independently selected from the group consisting of halide, —OMe, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl); and each p is independently 0 or 1; and

wherein each H atom is optionally independently replaced by ²H (D)(deuterium).

In another embodiment disclosed herein includes a compound having the structure of Formula I:

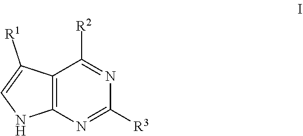

or a pharmaceutically acceptable salt thereof,

wherein:

R¹ is 8-11-membered bicyclic heteroaryl optionally substituted with 1-10 R⁴;

wherein a carbon atom on an aromatic ring of the heteroaryl forms the bond with

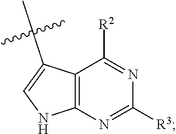

R² is selected from the group consisting of H, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(Cₗ₋ₛalkylene)OMe, —(C₁₋₃ alkylene)ₚcarbocyclyl optionally substituted with 1-12 R¹′, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁵;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁵;

each R²⁶ is independently selected from the group consisting of halide, —OR³², —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubst ituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D)(deuterium).

In another embodiment disclosed herein includes a compound having the structure of Formula I:

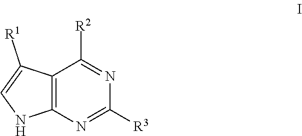

or a pharmaceutically acceptable salt thereof,

wherein:

R¹ is 8-11-membered bicyclic heteroaryl optionally substituted with 1-10 R⁴;

wherein a carbon atom on an aromatic ring of the heteroaryl forms the bond with

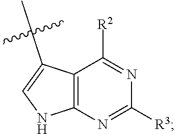

R² is selected from the group consisting of H, —OR⁷, 5-membered heteroaryl optionally substituted with 1-3 R⁸, and —NHR⁹,

R³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R¹⁸, —C(═O)N(R¹⁹)₂, and —C(═O)R²⁰;

alternatively, two R⁴ attached to the same carbon atom are taken together to form a carbonyl group;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R″;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅alkylene)OR³²;

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉alkyl), unsubstituted —(C₂₋₉ alkenyl), unsubstituted —(C₂₋₉ alkynyl), unsubstituted —(C₁₋₉ haloalkyl), and —heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁵;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁵;

each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);

each R³⁴ is independently selected from the group consisting of —N(R³⁷)₂, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D)(deuterium).

Some embodiments of the present disclosure include compounds of Formula I:

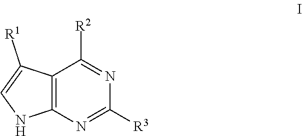

or salts, pharmaceutically acceptable salts, or prodrugs thereof,

wherein:

R¹ is selected from the group consisting of phenyl substituted with 1-5 R⁵, 6-membered

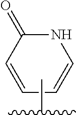

heteroaryl optionally substituted with 1-4 R⁶, and optionally substituted with 1-4 R⁶;

R² is selected from the group consisting of H, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

with the proviso that either R² or R³ is H but R² and R³ are not both H;

each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R¹⁸, —C(═O)N(R¹⁹)₂, and —C(═O)R²⁰;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 R^{16a}, -heteroaryl optionally substituted with 1-10 R^{17a}, —carbocyclyl optionally substituted with 1-12 R^{18a}, —C(═O)N(R^{19a})₂, and —C(═O)R^{20a};

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹′, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a;}

each R¹⁵′ is independently selected from the group consisting of unsubstituted —(C₁₋₉alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a};

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{16a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷′ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R¹⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R^{18a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R^{19a} are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R^{27a}, -heterocyclyl optionally substituted with 1-10 R^{28a}, and -heteroaryl optionally substituted with 1-10 R^{29a};

each R²⁰ is -heterocyclyl optionally substituted with 1-10 R²⁸;

each R^{20a} is -heterocyclyl optionally substituted with 1-10 R^{28a}; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{27a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{28a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(Cl_salkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula I is not a structure selected from the group consisting of:

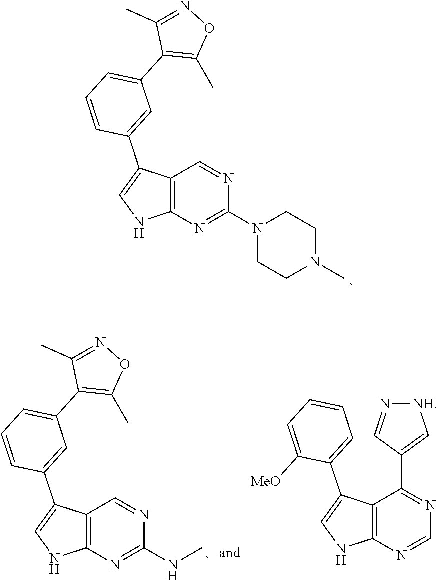 [SMILES: Cc1noc(C)c1-c1cccc(-c2c[nH]c3nc(N4CCN(C)CC4)ncc23)c1 ; CNc1ncc2c(-c3cccc(-c4c(C)noc4C)c3)c[nH]c2n1 ; COc1ccccc1-c1c[nH]c2ncnc(-c3cn[nH]c3)c12]

Some embodiments of the present disclosure include compounds of Formula I:

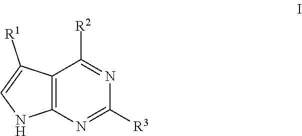

or salts, pharmaceutically acceptable salts, or prodrugs thereof,

wherein:

R¹ is selected from the group consisting of phenyl substituted with 1-5 R⁵, 6-membered

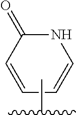

heteroaryl optionally substituted with 1-4 R⁶, and optionally substituted with 1-4 R⁶;

R² is selected from the group consisting of H, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

with the proviso that either R² or R³ is H but R² and R³ are not both H; each R⁵ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)₂, and —C(═O)R²⁰;

each R⁶ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 R^{16a}, -heteroaryl optionally substituted with 1-10 R^{17a}, —carbocyclyl optionally substituted with 1-12 R^{18a}, —C(═O)N(R¹⁹a)₂, and —C(═O)R^{20a};

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹; each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅alkylene)OR³²;

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R^{15a} is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a};

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{16a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R^{17a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{18a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R^{19a} are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R^{27a}, -heterocyclyl optionally substituted with 1-10 R^{28a}, and -heteroaryl optionally substituted with 1-10 R^{29a};

each R²⁰ is -heterocyclyl optionally substituted with 1-10 R²⁸;

each R^{20a} is -heterocyclyl optionally substituted with 1-10 R^{28a}; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{27a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{28a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C1-5 alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);

each R³⁴ is independently selected from the group consisting of —N(R³⁷)₂, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula I is not a structure selected from the group consisting of:

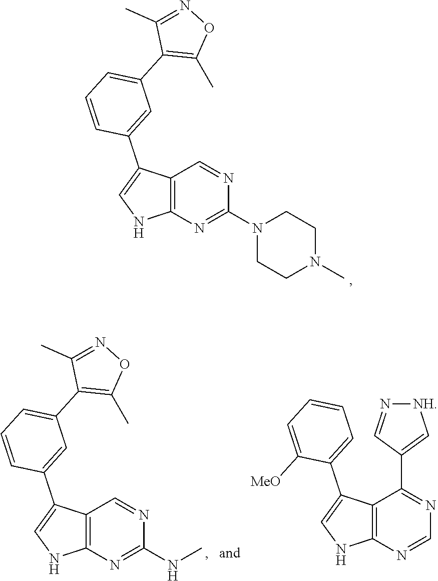 [SMILES: Cc1noc(C)c1-c1cccc(-c2c[nH]c3nc(N4CCN(C)CC4)ncc23)c1 ; CNc1ncc2c(-c3cccc(-c4c(C)noc4C)c3)c[nH]c2n1 ; COc1ccccc1-c1c[nH]c2ncnc(-c3cn[nH]c3)c12]

Some embodiments include stereoisomers and pharmaceutically acceptable salts of a compound of Formula I. Some embodiments include pharmaceutically acceptable salts of a compound of Formula I.

Some embodiments include pro-drugs of a compound of Formula I.

Some embodiments of the present disclosure include pharmaceutical compositions comprising a compound of Formula I and a pharmaceutically acceptable carrier, diluent, or excipient.

Other embodiments disclosed herein include methods of inhibiting DYRK1A by administering to a patient affected by a disorder or disease in which DYRK1A overexpression is implicated, such as Alzheimer's Disease, Amyotrophic Lateral Sclerosis, CDKLS Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor and Stroke.

Inhibitors of DYRK1A can also be used to treat tauopathies. Tauopathies are neurodegenerative disorders characterized by the deposition of abnormal tau protein in the brain. The spectrum of tau pathologies expands beyond the traditionally discussed disease forms like Pick's disease, progressive supranuclear palsy, corticobasal degeneration, and argyrophilic grain disease. Emerging entities and pathologies include globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia (Handbook of Clinical Neurology (2018), 145, 355-368 and Aging Cell (2019), 18(5), e 13000).

Inhibitors of DYRK1A can also be used to treat disorders associated with abnormal folate/methionine metabolism.

Non-limiting examples of diseases which can be treated with the compounds and compositions provided herein include a variety of cancers, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

Some embodiments of the present disclosure include methods to prepare compounds of Formula I.

It is to be understood that both the foregoing general description and the following detailed description are exemplary and explanatory only and are not restrictive of the disclosure, as claimed.

### DETAILED DESCRIPTION

Provided herein are compositions and methods for inhibiting DYRK1A.

Some embodiments provided herein relate to a method for treating a disease including, but not limited to, neurological diseases or disorders, cancers, cognitive deficits, knee osteoarthritis, tendinopathy, viral infections, unicellular parasite infections, and motor deficits.

In some embodiments, non-limiting examples of a neurological disease or disorder which can be treated with the compounds and compositions provided herein include, but are not limited to, Alzheimer's disease, amyotrophic lateral sclerosis, Down Syndrome, frontotemporal dementia with Parkinsonism-17 (FTDP-17), Lewy body dementia, Parkinson's disease, Pick's disease tauopathies, and additional diseases with pronounced neurodegeneration such as autism, dementia, epilepsy, Huntington's disease, multiple sclerosis; diseases and disorders associated with acquired brain injury such as Chronic Traumatic Encephalopathy, Traumatic Brain Injury, Tumor, and Stroke.

In some embodiments, non-limiting examples of cancers which can be treated with the compounds and compositions provided herein include solid cancers (e.g., glioblastoma, ovarian, breast, and pancreatic cancers) and leukemias (e.g., acute lymphoblastic leukemia, acute megakaryoblastic leukemia, and chronic myeloid leukemia).

In some embodiments, pharmaceutical compositions are provided that are effective for treatment of a disease of an animal, e.g., a mammal, caused by DYRK1A overexpression. The composition includes a pharmaceutically acceptable carrier and a compound as described herein.

Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-0—group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “haloalkoxy” means a haloalkyl-O—group in which the haloalkyl group is as described herein. Exemplary haloalkoxy groups include fluoromethoxy, difluoromethoxy, trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylalkylene” means an aryl-alkylene-group in which the aryl and alkylene moieties are as previously described. In some embodiments, arylalkylene groups contain a C₁₋₄alkylene moiety. Exemplary arylalkylene groups include benzyl and 2-phenethyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d] pyrimidinyl, pyrrolo[2,3-b] pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b] pyridinyl, tetrazolyl, chromane, 2,3-dihydrobenzo[b] [1,4]dioxine, benzo[d] [1,3]dioxole, 2,3-dihydrobenzofuran, tetrahydroquinoline, 2,3-dihydrobenzo [b] [1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched alkyl, alkenyl or alkynyl substituted with one or more chloro, bromo, fluoro, and/or iodo atom(s). In some embodiments, a haloalkyl is a fluoroalkyl, wherein one or more of the hydrogen atoms have been substituted by fluoro. In some embodiments, haloalkyls are of 1 to 3 carbons in length (e.g., 1 to 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings such as bicyclic and spirocyclic heterocyclyls. Heterocyclyls may be substituted or unsubstituted with one or more substituents. In some embodiments, heterocycles have 3-11 members. In six membered monocyclic heterocycles, the heteroatom(s) are selected from one to three of O, N and S, and wherein when the heterocycle is five membered, it can have one or two heteroatoms selected from O, N, and S. Examples of heterocyclyl include 2-azaspiro [3.5]nonanyl, 7-azaspiro[3.5]nonane, azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, 1,4-dioxaspirodecanyl, morpholinyl, thiomorpholinyl, piperazinyl, pyranyl, pyrrolidinyl, tetrahydrofuryl, tetrahydropyridinyl, octahydrocyclopenta[c]pyrrolyl, oxazinyl, 1-oxaspiro [3.5]nonanyl, 2-oxaspiro[3.5]nonanyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 3-7 members. In six membered monocyclic heterocycles, the heteroatom(s) are selected from one to three of O, N and S, and wherein when the heterocycle is five membered, it can have one or two heteroatoms selected from O, N, and S. Examples of monocyclic heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents. In some embodiments, bicyclic heterocycles have 4-11 members with the heteroatom(s) being selected from one to five of O, N and S. Examples of bicyclic heterocyclyls include 2-azabicyclo[1.1.0]butane, 2-azabicyclo [2.1.0]pentane, 2-azabicyclo [1.1.1]pentane, 3-azabicyclo [3.1.0] hexane, 5-azabicyclo [2.1.1]hexane, 3-azabicyclo [3 .2.0] heptane , octahydrocyclopenta[c]pyrrole, 3-azabicyclo [4.1.0] heptane, 7-azabicyclo [2.2.1]heptane, 6-azabicyclo [3.11]heptane, 7-azabicyclo[4.2.0]octane, 2-azabicyclo [2.2.2]octane, and the like.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N and S. Examples of spirocyclic heterocyclyls include 2-azaspiro[2.2]pentane, 4-azaspiro[2.5]octane, 1-azaspiro[3.5]nonane, 2-azaspiro [3.5]nonane, 7-azaspiro[3.5]nonane, 2-azaspiro[4.4]nonane, 6-azaspiro[2.6]nonane, 1,7-diazaspiro [4.5]decane, 2,5-diazaspiro [3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)2 and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R¹¹ are independently selected from H; —(C₁₋₉ alkyl); C6-10 aryl optionally substituted with 1-3 R′″; 5-10 membered heteroaryl having from 1-4 heteroatoms independently selected from N, O, and S and optionally substituted with 1-3 R′″; C₃₋₇ carbocyclyl optionally substituted with 1-3 R′″; and 3-8 membered heterocyclyl having from 1-4 heteroatoms independently selected from N, 0, and S and optionally substituted with 1-3 R″; wherein each R′″ is independently selected from —(C₁₋₆ alkyl), —(C1-6 haloalkyl), a halide (e.g., F), a hydroxyl, —C(O)OR, —C(O)R, —(C₁₋₆ alkoxyl), —NRR′, —C(O)NRR', and a cyano, in which each occurrence of R and R′ is independently selected from H and —(C₁₋₆ alkyl). In some embodiments, the substituent is selected from —(C₁₋₆ alkyl), 4C₁-6 haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formulas I, Ia, Ib, and Ic, wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), isotopes of carbon, such as ¹¹C, ¹³C and ¹⁴C, isotopes of chlorine, such as ³⁶C1, isotopes of fluorine, such as ¹⁸F, isotopes of iodine, such as ¹²³1 and ¹²1, isotopes of nitrogen, such as ¹³N and ¹⁵N, isotopes of oxygen, such as ¹⁵O, ¹⁷O and ¹⁸O, isotopes of phosphorus, such as ³²P, and isotopes of sulfur, such as ³⁵S.

The term “administration” or “administering” refers to a method of providing a dosage of a compound or pharmaceutical composition to a vertebrate or invertebrate, including a mammal, a bird, a fish, or an amphibian, where the method of administration is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” and “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formulas I, Ia, Ib, and Ic, in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age, and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing, or preventing the further development of a disorder, and/or reducing the severity of symptoms that will or are expected to develop.

“Drug-eluting” and/or controlled release as used herein refers to any and all mechanisms, e.g., diffusion, migration, permeation, and/or desorption by which the drug(s) incorporated in the drug-eluting material pass therefrom over time into the surrounding body tissue.

“Drug-eluting material” and/or controlled release material as used herein refers to any natural, synthetic, or semi-synthetic material capable of acquiring and retaining a desired shape or configuration and into which one or more drugs can be incorporated and from which incorporated drug(s) are capable of eluting over time.

“Elutable drug” as used herein refers to any drug or combination of drugs having the ability to pass over time from the drug-eluting material in which it is incorporated into the surrounding areas of the body.

Compounds

The compounds and compositions described herein can be used to inhibit DYRK1A for treating a disorder or disease in which DYRK1A overexpression is implicated, such as in neurological diseases or disorders, cancers, cognitive deficits, knee osteoarthritis, tendinopathy, viral infections, unicellular parasite infections, and motor deficits.

Some embodiments of the present disclosure include compounds of Formula I:

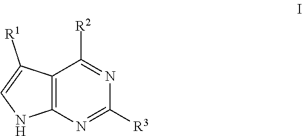

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the heteroaryl forms the bond with

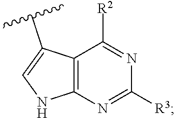

R² is selected from the group consisting of H, —OR⁷, 5-membered heteroaryl optionally substituted with 1-3 R⁸, and —NHR⁹;

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

with the proviso that R² and R³ are not both H;

each R⁴ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₃ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉alkyl), unsubstituted —(C₂₋₉ alkenyl), unsubstituted —(C₂₋₉ alkynyl), unsubstituted —(C₁₋₉ haloalkyl), and —heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸;

each R²¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²² is selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁶;

each R²⁶ is independently selected from the group consisting of halide, —OMe, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each 12_³¹ is independently selected from the group consisting of unsubstituted —(C₁₋₉alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and with the proviso that Formula I is not a structure selected from the group consisting of:

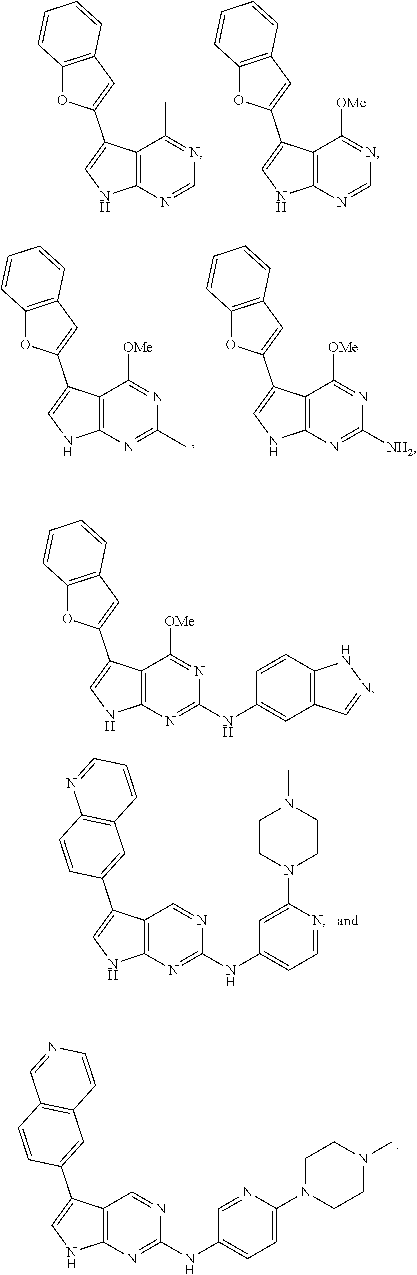

Some embodiments of the present disclosure include compounds of Formula I:

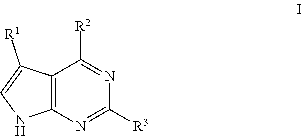

or salts, pharmaceutically acceptable salts, or prodrugs thereof, wherein:

R¹ is 8-11-membered bicyclic heteroaryl optionally substituted with 1-10 R⁴;

wherein a carbon atom on an aromatic ring of the heteroaryl forms the bond with

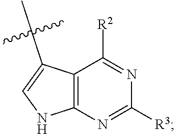

R² is selected from the group consisting of H, 5-membered heteroaryl optionally substituted with 1-3 R⁸, and —NHR⁹;

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —heterocyclyl optionally substituted with 1-10 R¹¹, —(5-10 membered heteroaryl) optionally substituted with 1-10 R¹², —(CH₂)ₚcarbocyclyl optionally substituted with 1-12 R¹², and —NHR¹⁴;

with the proviso that R² and R³ are not both H;

each R⁴ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁶;

each R²⁵ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁶;

each R²⁶ is independently selected from the group consisting of halide, —01e, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅alkylene)01e²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula I is not a structure selected from the group consisting of:

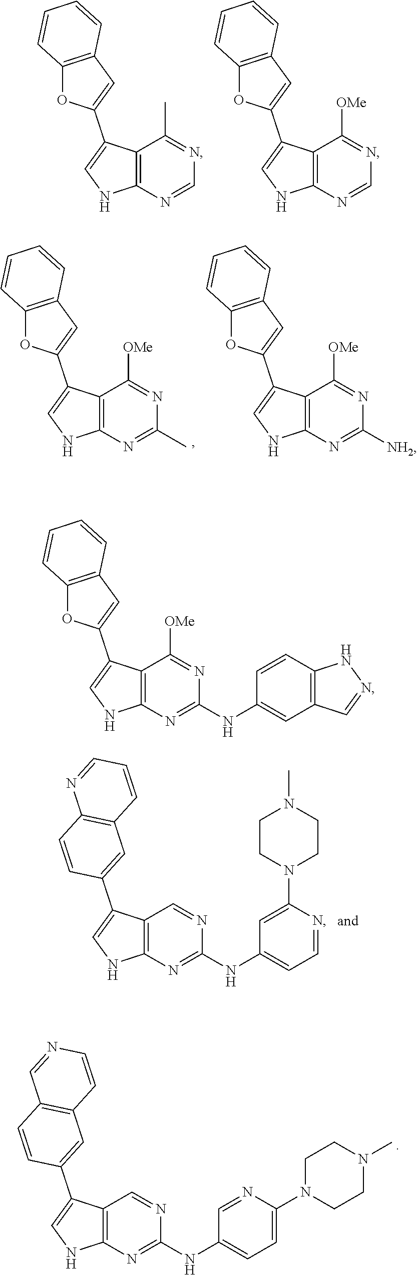

Some embodiments of the present disclosure include compounds of Formula

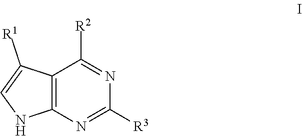

or salts, pharmaceutically acceptable salts, or prodrugs thereof,

wherein:

R¹ is 8-11-membered bicyclic heteroaryl optionally substituted with 1-10 R⁴; wherein a carbon atom on an aromatic ring of the heteroaryl forms the bond with

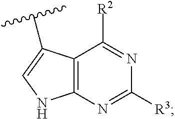

R² is selected from the group consisting of H, 5-membered heteroaryl optionally substituted with 1-3 R⁸, and —NHR⁹;

R³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹¹, and —NHR¹⁴;

with the proviso that R² and R³ are not both H;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R″;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸;

each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R¹⁶;

each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(R³⁷)2, and -heterocyclyl optionally substituted with 1-10 R²⁸;

each R³⁵ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula I is not a structure selected from the group consisting of:

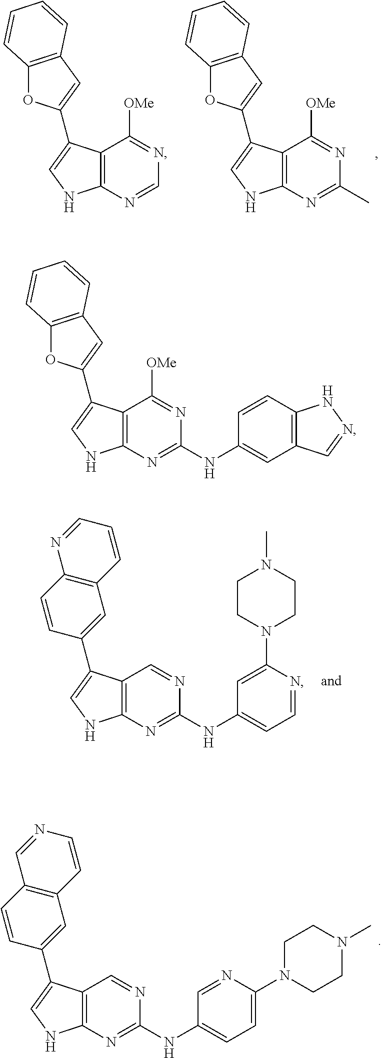 [SMILES: COc1ncnc2[nH]cc(-c3cc4ccccc4o3)c12 ; COc1nc(C)nc2[nH]cc(-c3cc4ccccc4o3)c12 ; COc1nc(Nc2ccc3[nH]ncc3c2)nc2[nH]cc(-c3cc4ccccc4o3)c12 ; CN1CCN(c2cc(Nc3ncc4c(-c5ccc6ncccc6c5)c[nH]c4n3)ccn2)CC1 ; CN1CCN(c2ccc(Nc3ncc4c(-c5ccc6cnccc6c5)c[nH]c4n3)cn2)CC1]

Some embodiments of the present disclosure include compounds of Formula I:

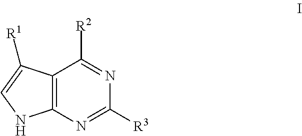

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the heteroaryl forms the bond with

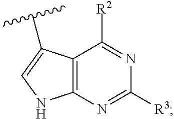

R² is —OW;

R³ is —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, —carbocyclyl optionally substituted with 1-12 R⁸, —C(═O)N(R¹⁹)₂, and —C(═O)R²⁰;

R⁷ is selected from the group consisting of unsubstituted —(C₁₋₃ alkyl) and unsubstituted —(C₁₋₃ haloalkyl);

R¹⁴ is selected from the group consisting of —carbocyclyl optionally substituted with 1-12 R¹⁷ and -heterocyclyl optionally substituted with 1-10 R²⁶;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁰ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(R³⁷)₂, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D) (deuterium).

Some embodiments of the present disclosure include compounds of Formula

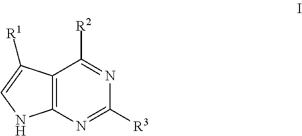

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the heteroaryl forms the bond with

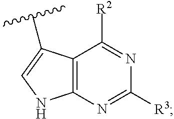

R² is —NHR⁹;

R³ is —NHR¹⁴;

each R⁴ is independently selected from the group consisting of halide, unsubstituted —(C₁₋₉alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, —carbocyclyl optionally substituted with 1-12 R⁸, —C(═O)N(R¹⁹)2, and —C(═O)R²⁰;

R⁹ is selected from the group consisting of unsubstituted —(C₁₋₃ alkyl) and unsubstituted —(C₁₋₃ haloalkyl);

R¹⁴ is selected from the group consisting of —carbocyclyl optionally substituted with 1-12 R¹' and -heterocyclyl optionally substituted with 1-10 R²⁶;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

h R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C_{1—}salkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(R³⁷)2, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D) (deuterium).

Some embodiments of the present disclosure include compounds of Formula

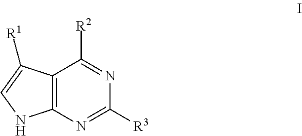

or salts, pharmaceutically acceptable salts, or prodrugs thereof,

wherein:

R¹ is selected from the heteroaryl group consisting of:

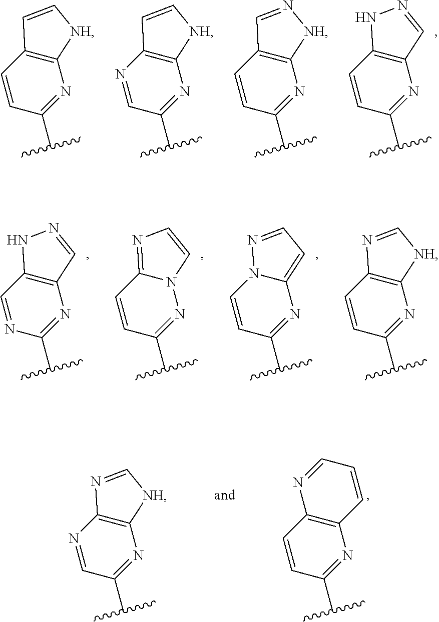

optionally substituted with 1-10 R⁴;

R² is —NHR⁹;

R³ is —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, —carbocyclyl optionally substituted with 1-12 R⁸, —C(═O)N(R¹⁹)2, and —C(═O)R²⁰;

R⁹ is selected from the group consisting of unsubstituted —(C₁₋₃ alkyl) and unsubstituted —(C₁₋₃ haloalkyl);

R¹⁴ is selected from the group consisting of —carbocyclyl optionally substituted with 1-12 R¹⁷ and -heterocyclyl optionally substituted with 1-10 R²⁶;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(R³⁷)₂, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D) (deuterium).

Some embodiments of the present disclosure include compounds of Formula Ia:

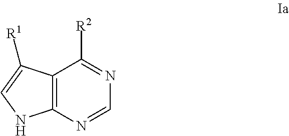

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the heteroaryl forms the bond with

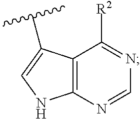

R² is selected from the group consisting of H, 5-membered heteroaryl optionally substituted with 1-3 R⁸, and —NHR⁹;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)2, and —C(═O)R²⁰ ;

alternatively, two R⁴ attached to the same carbon atom are taken together to form a carbonyl group;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and with the proviso that Formula Ia is not a structure selected from the group consisting of:

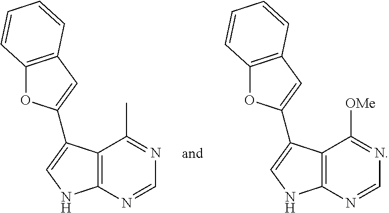 [SMILES: Cc1ncnc2[nH]cc(-c3cc4ccccc4o3)c12 ; COc1ncnc2[nH]cc(-c3cc4ccccc4o3)c12]

Some embodiments of the present disclosure include compounds of Formula Ia:

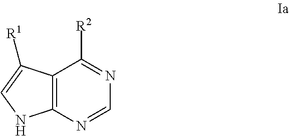

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the heteroaryl forms the bond with

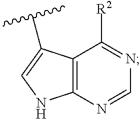

R² is selected from the group consisting of H, 5-membered heteroaryl optionally substituted with 1-3 R⁸, and —NHR⁹;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R¹⁸, —C(═O)N(R¹⁹)2, and —C(═O)R²⁰;

alternatively, two R⁴ attached to the same carbon atom are taken together to form a carbonyl group;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸;

each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(Cl_salkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; wherein each H atom is optionally independently replaced by ²H (D) (deuterium); and with the proviso that Formula Ia is not a structure selected from the group consisting of:

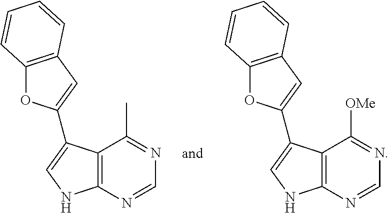 [SMILES: Cc1ncnc2[nH]cc(-c3cc4ccccc4o3)c12 ; COc1ncnc2[nH]cc(-c3cc4ccccc4o3)c12]

Some embodiments of the present disclosure include compounds of Formula Ia:

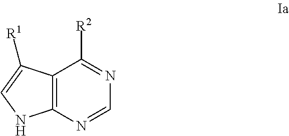

or salts, pharmaceutically acceptable salts, or prodrugs thereof,

wherein:

R¹ is 8-11-membered bicyclic heteroaryl optionally substituted with 1-10 R⁴; wherein a carbon atom on an aromatic ring of the heteroaryl forms the bond with

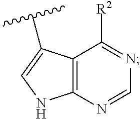

R² is selected from the group consisting of —OR', 5-membered heteroaryl optionally substituted with 1-3 R⁸, and —NHR⁹;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C1-5 alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);

each R³⁴ is independently selected from the group consisting of —N(R³⁷)₂, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula Ia is not a structure selected from the group consisting of:

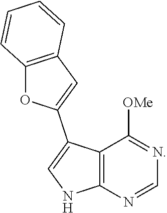 [SMILES: COc1ncnc2[nH]cc(-c3cc4ccccc4o3)c12]

Some embodiments of the present disclosure include compounds of Formula Ia:

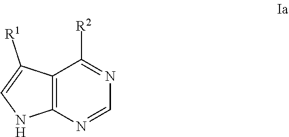

or salts, pharmaceutically acceptable salts, or prodrugs thereof,

wherein:

R¹ is selected from the heteroaryl group consisting of:

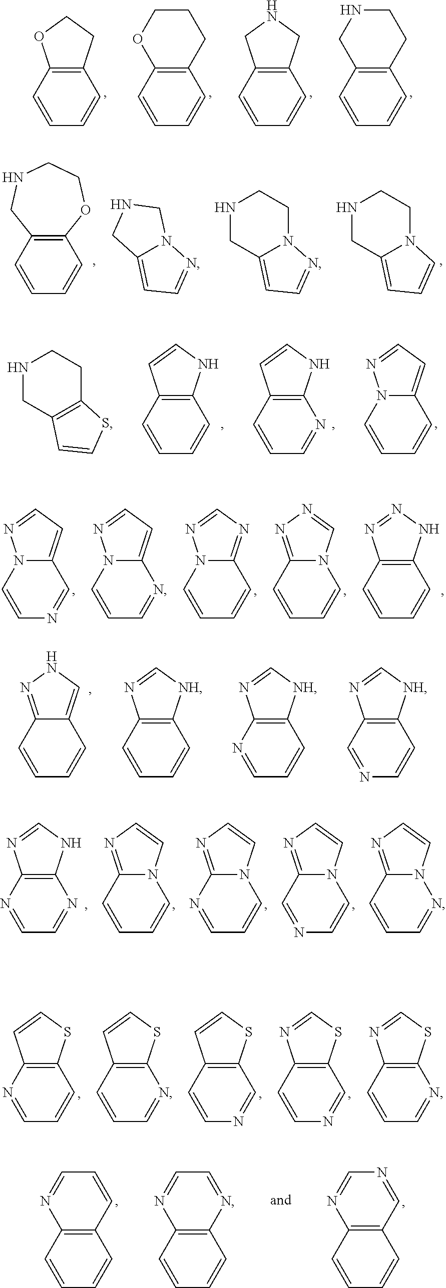

optionally

substituted with 1-10 R⁴;

wherein a carbon atom on an aromatic ring of the heteroaryl forms the bond with

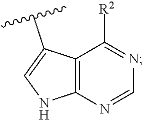

R² is selected from the group consisting of H, 5-membered heteroaryl optionally substituted with 1-3 R⁸, and —NHR⁹;

each R⁴ is independently selected from the group consisting of halide, unsubstituted —(C₁₋₉ 9alkyl), unsubstituted —(C₂₋₉ alkenyl), unsubstituted —(C₂₋₉alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)₂, and —C(═O)R²⁰;

alternatively, two R⁴ attached to the same carbon atom are taken together to form a carbonyl group;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each 12_³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and wherein each H atom is optionally independently replaced by ²H (D) (deuterium).

Some embodiments of the present disclosure include compounds of Formula Ia:

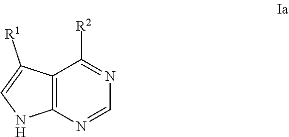

or salts, pharmaceutically acceptable salts, or prodrugs thereof,

wherein:

R¹ is selected from the heteroaryl group consisting of:

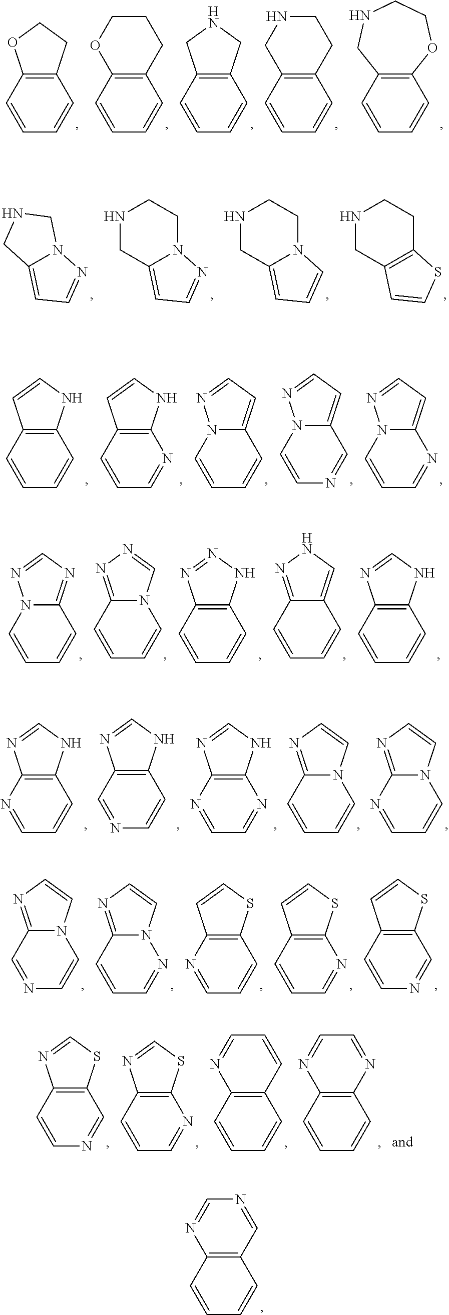

optionally

substituted with 1-10 R⁴;

wherein a carbon atom on an aromatic ring of the heteroaryl forms the bond with

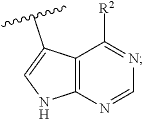

R² is selected from the group consisting of H, 5-membered heteroaryl optionally substituted with 1-3 R⁸, and —NHR⁹;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(Cl_salkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D) (deuterium).

Some embodiments of the present disclosure include compounds of Formula Ia:

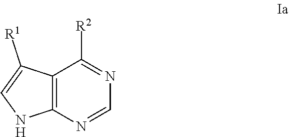

or salts, pharmaceutically acceptable salts, or prodrugs thereof,

wherein:

R¹ is selected from the heteroaryl group consisting of:

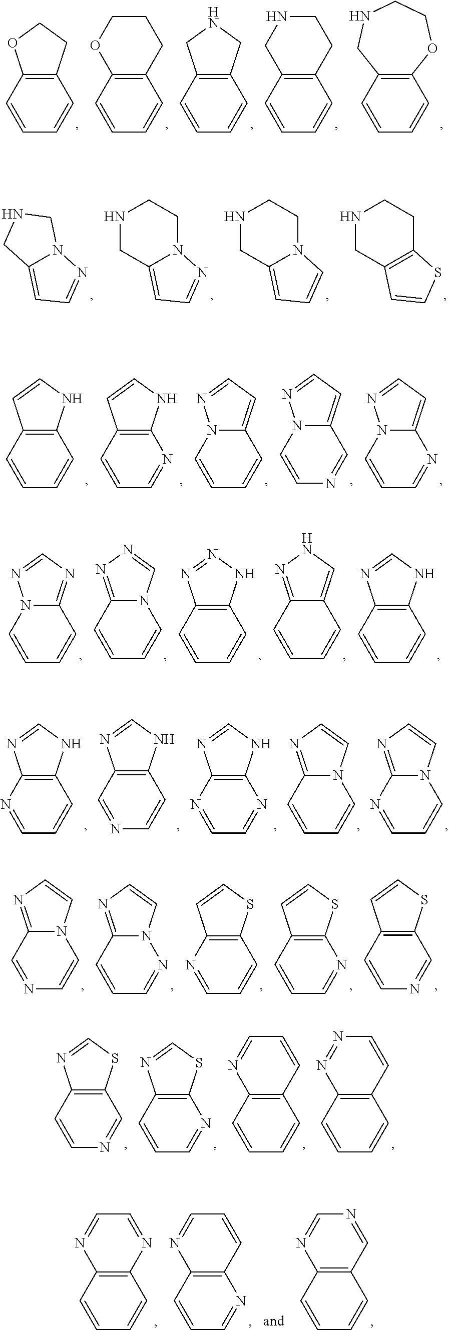

optionally substituted with 1-10 R⁴;

wherein a carbon atom on an aromatic ring of the heteroaryl forms the bond with

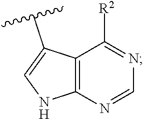

R² is selected from the group consisting of —OR', 5-membered heteroaryl optionally substituted with 1-3 R⁸, and —NHR⁹;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶; each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸;

each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C1-5 alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);

each R³⁴ is independently selected from the group consisting of —N(R³⁷)₂, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D) (deuterium).

Some embodiments of the present disclosure include compounds of Formula Ib:

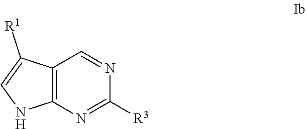

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the bicyclic heteroaryl form the bond with

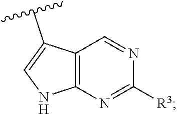

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)₂, and —C(═O)R²⁰;

alternatively, two R⁴ attached to the same carbon atom are taken together to form a carbonyl group;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₃ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁴ is s independently elected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OMe, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula Ib is not a structure selected from the group consisting of:

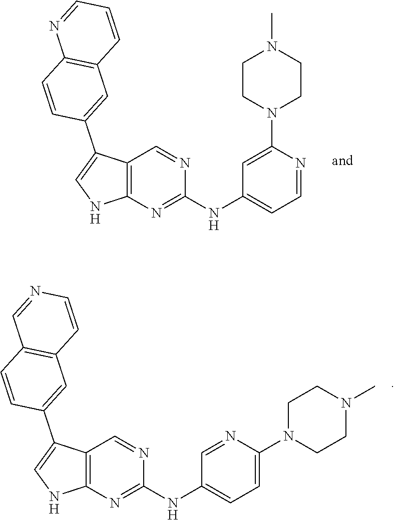 [SMILES: CN1CCN(c2cc(Nc3ncc4c(-c5ccc6ncccc6c5)c[nH]c4n3)ccn2)CC1 ; CN1CCN(c2ccc(Nc3ncc4c(-c5ccc6cnccc6c5)c[nH]c4n3)cn2)CC1]

Some embodiments of the present disclosure include compounds of Formula Ib:

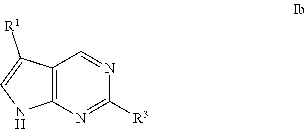

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the bicyclic heteroaryl form the bond with

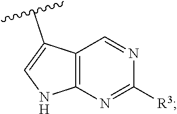

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(Cₗ₋ₛalkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸;

each R²⁴ is s independently elected from the group consisting of halide, unsubstituted —(C alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³², —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula Ib is not a structure selected from the group consisting of:

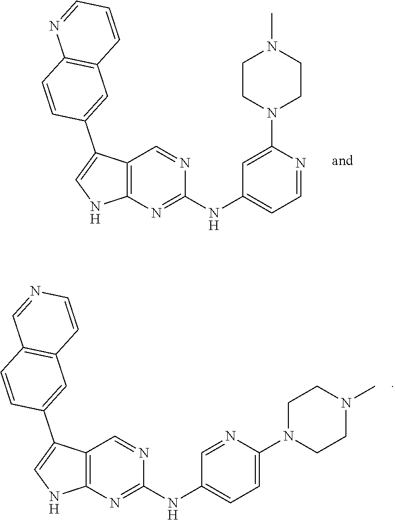 [SMILES: CN1CCN(c2cc(Nc3ncc4c(-c5ccc6ncccc6c5)c[nH]c4n3)ccn2)CC1 ; CN1CCN(c2ccc(Nc3ncc4c(-c5ccc6cnccc6c5)c[nH]c4n3)cn2)CC1]

Some embodiments of the present disclosure include compounds of Formula Ib:

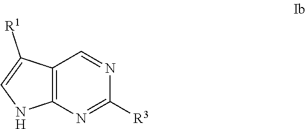

or salts, pharmaceutically acceptable salts, or prodrugs thereof,

wherein:

R¹ is 8-11-membered bicyclic heteroaryl optionally substituted with 1-10 R⁴;

wherein a carbon atom on an aromatic ring of the bicyclic heteroaryl form the bond with

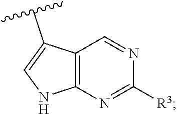

R³ is selected from the group consisting of —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R12, (CH₂)_{pcar}b_{ocyc}l_{yl opt}iₒₙₐll_{y} substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{il} is independently selected from the group consisting of halide, unsubstituted —(C alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R¹² is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl); each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁴ is s independently elected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(Cl_salkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each leis independently selected from the group consisting of —N(R³⁷)2, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula Ib is not a structure selected from the group consisting of:

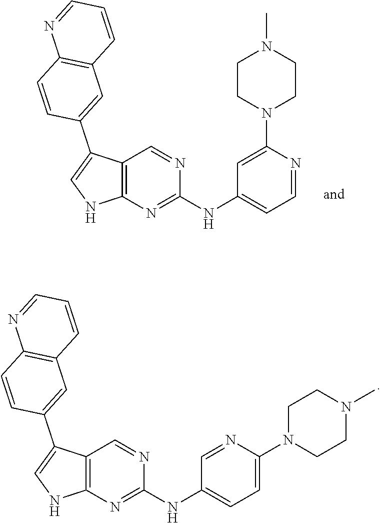 [SMILES: CN1CCN(c2cc(Nc3ncc4c(-c5ccc6ncccc6c5)c[nH]c4n3)ccn2)CC1 ; CN1CCN(c2ccc(Nc3ncc4c(-c5ccc6ncccc6c5)c[nH]c4n3)cn2)CC1]

Some embodiments of the present disclosure include compounds of Formula Ib:

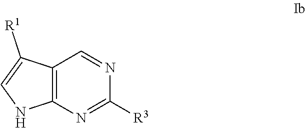

or salts, pharmaceutically acceptable salts, or prodrugs thereof,

wherein:

R¹ is selected from the heteroaryl group consisting of:

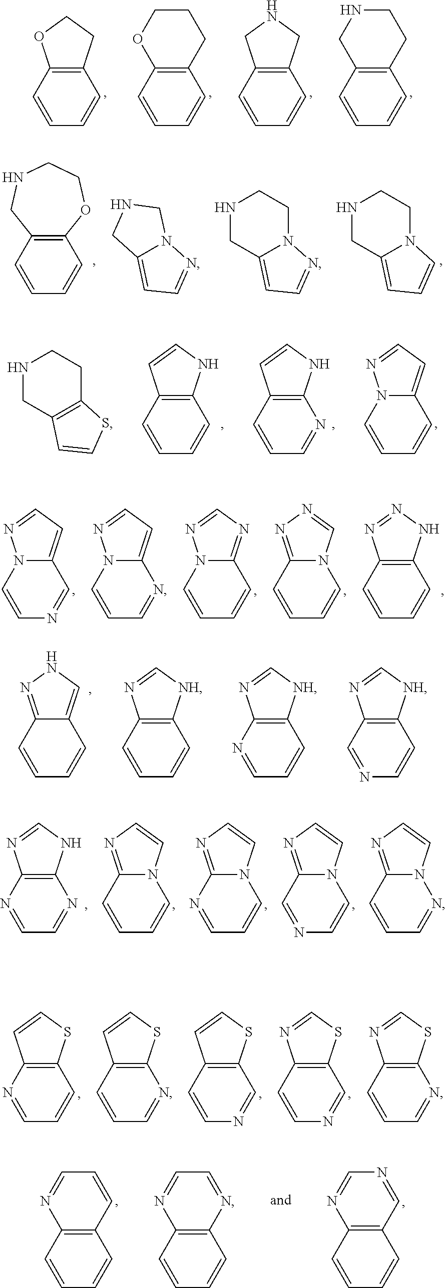

optionally

substituted with 1-10 R⁴;

wherein a carbon atom on an aromatic ring of the heteroaryl forms the bond with

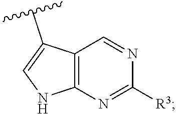

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₃ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OMe, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C alkyl), unsubstituted —(C₂₋₉ alkenyl), unsubstituted —(C₂₋₉ alkynyl), and unsubstituted —(CI-9 haloalkyl);

each 12_³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula Ib is not a structure selected from the group consisting of:

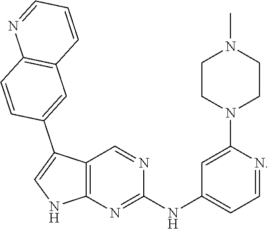 [SMILES: CN1CCN(c2cc(Nc3ncc4c(-c5ccc6ncccc6c5)c[nH]c4n3)ccn2)CC1]

Some embodiments of the present disclosure include compounds of Formula Ib:

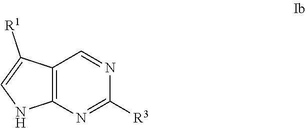

or salts, pharmaceutically acceptable salts, or prodrugs thereof,

wherein:

R¹ is selected from the heteroarvl group consisting of:

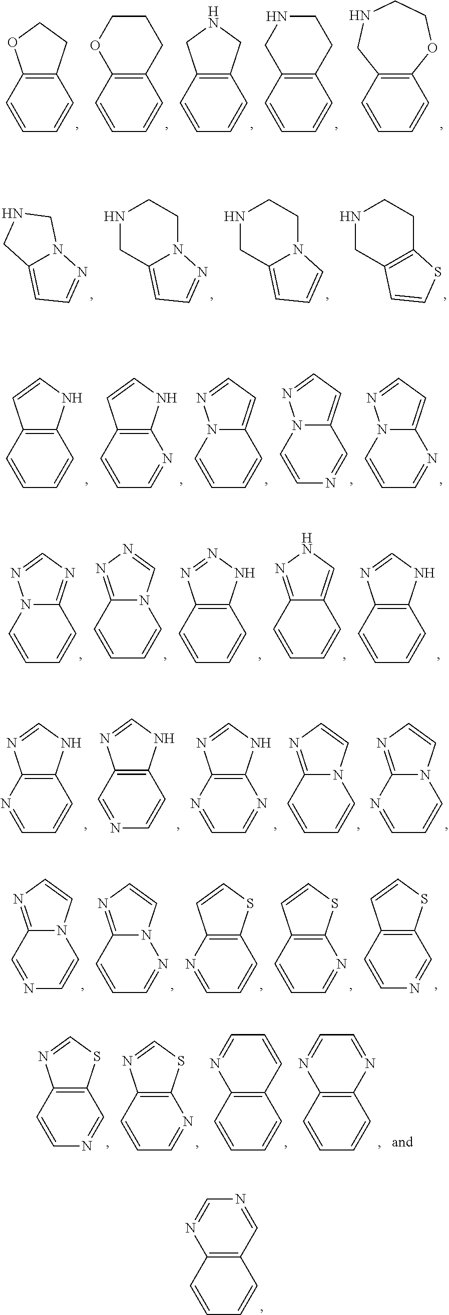

optionally

substituted with 1-10 R⁴;

wherein a carbon atom on an aromatic ring of the heteroaryl forms the bond with

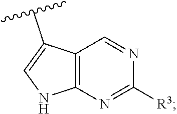

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹' is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁴ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³², —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR²²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula Ib is not a structure selected from the group consisting of:

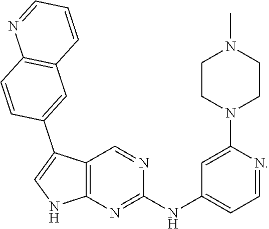 [SMILES: CN1CCN(c2cc(Nc3ncc4c(-c5ccc6ncccc6c5)c[nH]c4n3)ccn2)CC1]

Some embodiments of the present disclosure include compounds of Formula Ib:

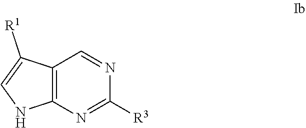

or salts, pharmaceutically acceptable salts, or prodrugs thereof,

wherein:

R¹ is selected from the heteroaryl group consisting of:

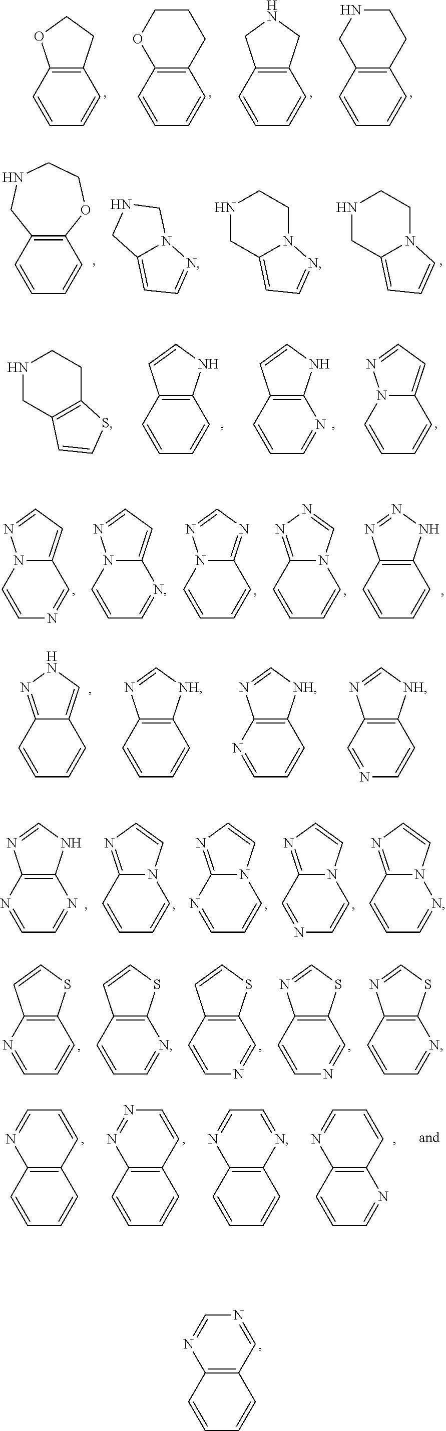

optionally substituted with 1-10 R⁴;

wherein a carbon atom on an aromatic ring of the heteroaryl forms the bond with

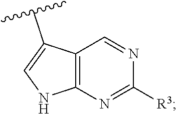

R³ is selected from the group consisting of —OR¹⁻®, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹²,

(CH₂)ₚcarbocyclyl optionally substituted with 1-12 R¹¹, and —NHR¹⁴;

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(Cl_salkylene)OR³²;

each R¹² is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³², —C(═O)12_³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(23⁷)2, and -heterocyclyl optionally substituted with 1-10 R²⁸;

each R³⁵ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and with the proviso that Formula Ib is not a structure selected from the group consisting of:

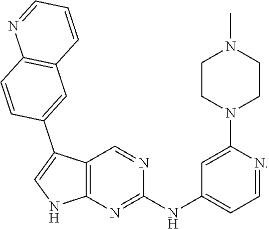 [SMILES: CN1CCN(c2cc(Nc3ncc4c(-c5ccc6ncccc6c5)c[nH]c4n3)ccn2)CC1]

Some embodiments of the present disclosure include compounds of Formula Ic:

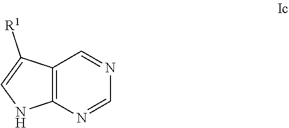

or salts, pharmaceutically acceptable salts, or prodrugs thereof,

wherein:

R¹ is selected from the heteroaryl group consisting of:

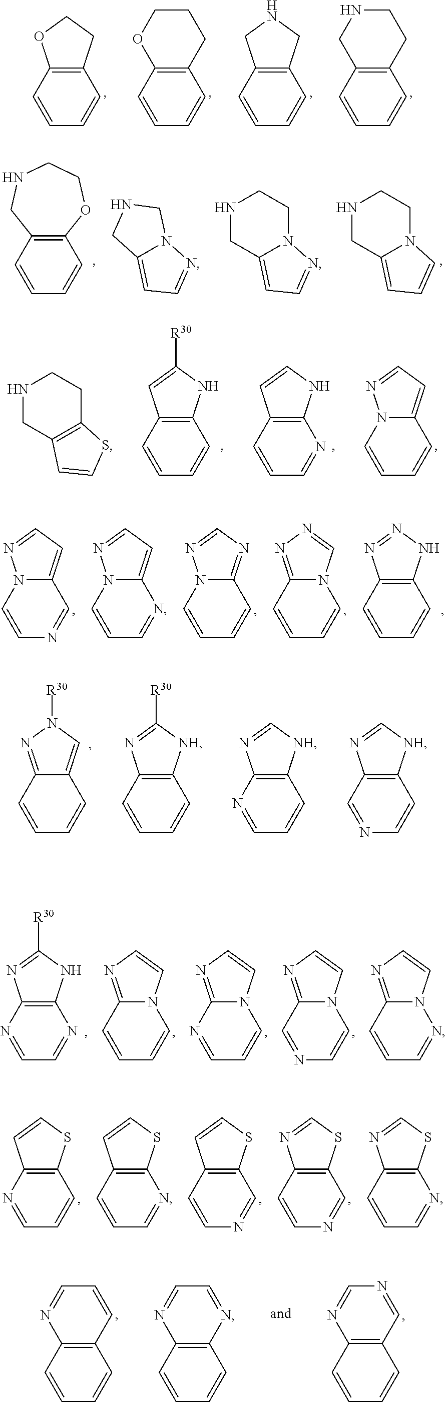

optionally

substituted with 1-10 R⁴;

wherein a carbon atom on an aromatic ring of the heteroaryl forms the bond with

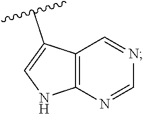 [SMILES: *c1c[nH]c2ncncc12]

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)₂, and —C(═O)R²⁰;

alternatively, two R⁴ attached to the same carbon atom are taken together to form a carbonyl group;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —01V¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R^{N} is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each 12_³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and wherein each H atom is optionally independently replaced by ²H (D) (deuterium).

Some embodiments of the present disclosure include compounds of Formula Ic:

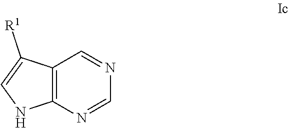

or salts, pharmaceutically acceptable salts, or prodrugs thereof,

wherein:

R¹ is selected from the heteroaryl group consisting of:

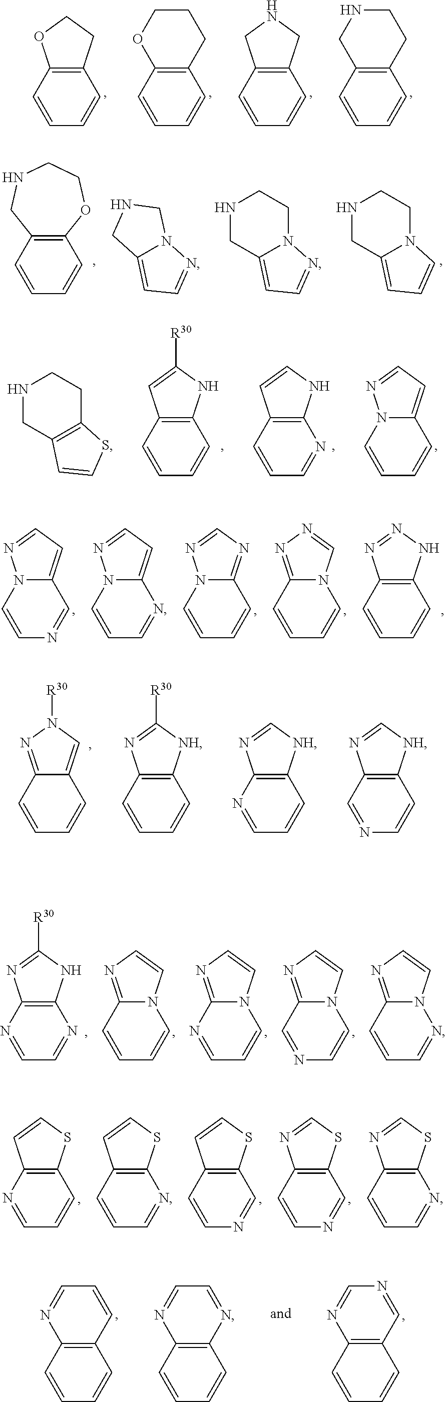

optionally

substituted with 1-10 R⁴;

wherein a carbon atom on an aromatic ring of the heteroaryl forms the bond with

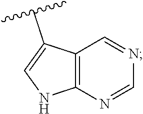 [SMILES: *c1c[nH]c2ncncc12]

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R¹¹, —C(═O)N(R¹⁹)2, and —C(═O)R²⁰;

alternatively, two R⁴ attached to the same carbon atom are taken together to form a carbonyl group;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{N} is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(Cl_salkylene)OR2²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and wherein each H atom is optionally independently replaced by ²H (D) (deuterium).

Some embodiments of the present disclosure include compounds of Formula Ic:

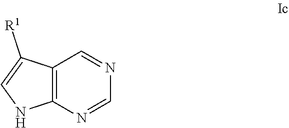

or salts, pharmaceutically acceptable salts, or prodrugs thereof,

wherein:

R¹ is selected from the heteroaryl group consisting of:

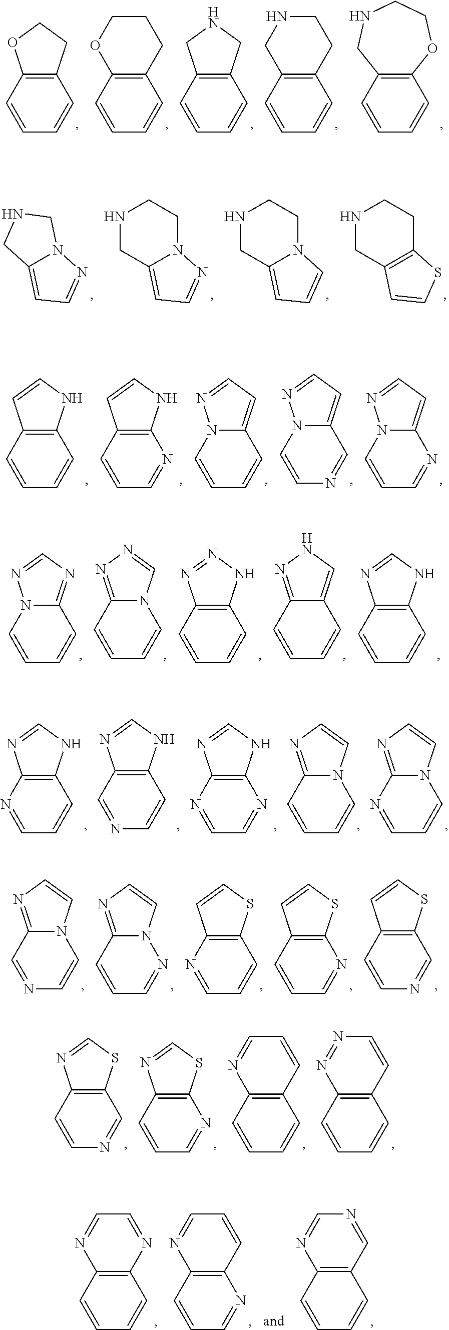

optionally substituted with 1-10 R⁴;

wherein a carbon atom on an aromatic ring of the heteroaryl forms the bond with

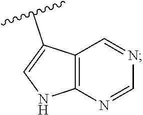 [SMILES: *c1c[nH]c2ncncc12]

each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)2, and —C(═O)R²⁰;

alternatively, two R⁴ attached to the same carbon atom are taken together to form a carbonyl group;

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and heterocyclyl optionally substituted with 1-10 R¹⁶;

each R¹⁶ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R²⁰ is -heterocyclyl optionally substituted with 1-10 R²⁸; each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R³° is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(R³⁷)2, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; and

wherein each H atom is optionally independently replaced by ²H (D) (deuterium).

Some embodiments of the present disclosure include compounds of Formula

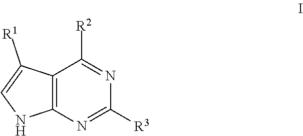

or salts, pharmaceutically acceptable salts, or prodrugs thereof,

wherein:

R¹ is selected from the group consisting of phenyl substituted with 1-5 R⁵, 6-membered

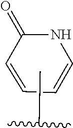

heteroaryl optionally substituted with 1-4 R⁶, and optionally substituted with 1-4 R⁶;

R² is selected from the group consisting of H, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;

with the proviso that either R² or R³ is H but R² and R³ are not both H; each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹′, —carbocyclyl optionally substituted with 1-12 R⁸, —C(═O)N(R¹⁹)2, and —C(═O)R²⁰;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 R^{16a}, -heteroaryl optionally substituted with 1-10 R^{17a}, —carbocyclyl optionally substituted with 1-12 R^{18a}, —C(═O)N(R¹⁹a)₂, ₐₙd —C(═O)R²⁰ ®;

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₃ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁵′ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a};

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{16a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{17a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{18a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R^{19a} are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R^{27a}, -heterocyclyl optionally substituted with 1-10 R^{28a}, and -heteroaryl optionally substituted with 1-10 R^{29a};

each R²⁰ is -heterocyclyl optionally substituted with 1-10 R²⁸; each R^{26a} is -heterocyclyl optionally substituted with 1-10 R^{28a}; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶; each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OMe, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{27a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{28a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1; wherein each H atom is optionally independently replaced by ²H (D) (deuterium); and with the proviso that Formula I is not a structure selected from the group consisting of:

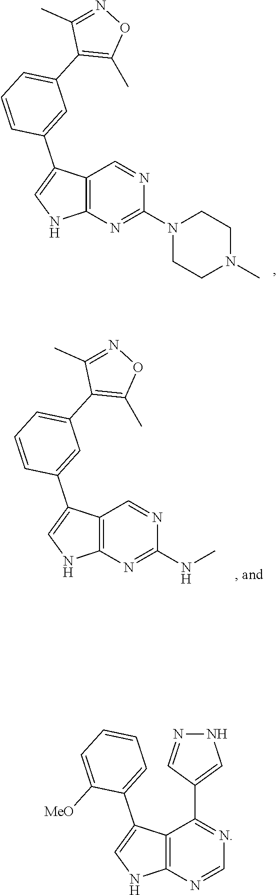 [SMILES: Cc1noc(C)c1-c1cccc(-c2c[nH]c3nc(N4CCN(C)CC4)ncc23)c1 ; CNc1ncc2c(-c3cccc(-c4c(C)noc4C)c3)c[nH]c2n1 ; COc1ccccc1-c1c[nH]c2ncnc(-c3cn[nH]c3)c12]

Some embodiments of the present disclosure include compounds of Formula I:

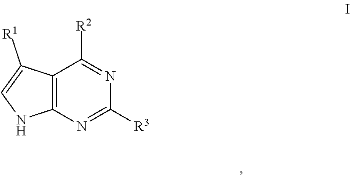

or salts, pharmaceutically acceptable salts, or prodrugs thereof, wherein:

R¹ is selected from the group consisting of phenyl substituted with 1-5 R⁵, 6-membered

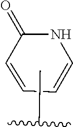

heteroaryl optionally substituted with 1-4 R⁶, and optionally substituted with 1-4 R⁶;

R² is selected from the group consisting of H, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹¹, and —NHR¹⁴;

with the proviso that either R² or R³ is H but R² and R³ are not both H; each R⁵ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹', —carbocyclyl optionally substituted with 1-12 R⁸, —C(═O)N(R¹⁹)₂, and —C(═O)R²⁰;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 R^{16a}, -heteroaryl optionally substituted with 1-10 R^{17a}, —carbocyclyl optionally substituted with 1-12 R^{18a}, —C(═O)N(R¹⁹a)₂, and —C(═O)R^{26a};

127 is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁶ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹′, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R^{15a} is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a};

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{16a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{17a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸′ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R¹⁹′ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R^{27a}, -heterocyclyl optionally substituted with 1-10 R^{28a}, and -heteroaryl optionally substituted with 1-10 R^{29a};

each R²⁰ is -heterocyclyl optionally substituted with 1-10 R²⁸; each R²⁰′ is -heterocyclyl optionally substituted with 1-10 R^{28a}; each R²¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —01e, —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{27a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{28a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C,_salkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula I is not a structure selected from the group consisting of:

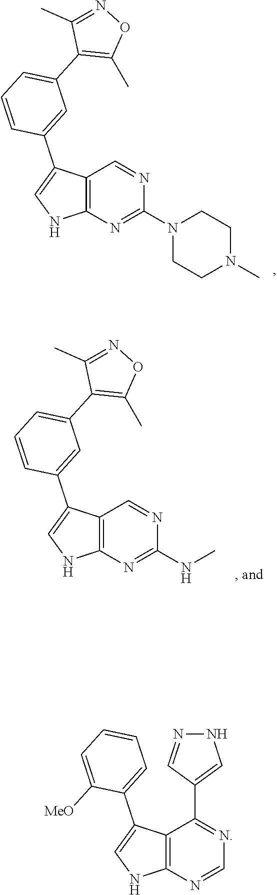 [SMILES: Cc1noc(C)c1-c1cccc(-c2c[nH]c3nc(N4CCN(C)CC4)ncc23)c1 ; CNc1ncc2c(-c3cccc(-c4c(C)noc4C)c3)c[nH]c2n1 ; COc1ccccc1-c1c[nH]c2ncnc(-c3cn[nH]c3)c12]

Some embodiments of the present disclosure include compounds of Formula

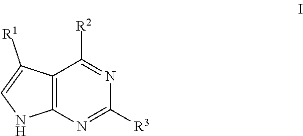

or salts, pharmaceutically acceptable salts, or prodrugs thereof, wherein:

R¹ is selected from the group consisting of phenyl substituted with 1-5 R⁵, 6-membered

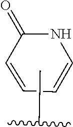

heteroaryl optionally substituted with 1-4 R⁶, and optionally substituted with 1-4 R⁶;

R² is selected from the group consisting of H, 5-membered heteroaryl optionally substituted with 1-3 R⁸, and —NHR⁹,

R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹², and —NHR¹⁴; with the proviso that either R² or R³ is H but R² and R³ are not both H; each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 R¹⁶′, -heteroaryl optionally substituted with 1-10 R^{17a}, —carbocyclyl optionally substituted with 1-12 R^{18a}, —C(═O)N(R¹⁹′)₂, and —C(═O)R²⁰ ′;

R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;

each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C alkyl), unsubstituted —(C₂₋₉ alkenyl), unsubstituted —(C₂₋₉ alkynyl), unsubstituted —(C₁₋₉ haloalkyl), and —(C1-5 alkylene)OR³²;

each R¹² is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);

each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R¹⁵′ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a};

each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{16a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;

each R¹⁷′ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁸′ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;

each R^{19a} are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R^{27a}, -heterocyclyl optionally substituted with 1-10 R^{28a}, and -heteroaryl optionally substituted with 1-10 R^{29a};

each R²⁰ is -heterocyclyl optionally substituted with 1-10 R²⁸; each R²⁰′ is -heterocyclyl optionally substituted with 1-10 R^{28a}; each R²¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;

each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;

alternatively, two R²⁶ attached to the same carbon atom are taken together to form a carbonyl group;

each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R^{27a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R^{28a} is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);

each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;

each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁴ is independently selected from the group consisting of —N(R³⁷)2, and -heterocyclyl optionally substituted with 1-10 R²⁸;

each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁵ attached to the same carbon atom are taken together to form a carbonyl group;

each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);

each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each p is independently 0 or 1;

wherein each H atom is optionally independently replaced by ²H (D) (deuterium); and

with the proviso that Formula I is not a structure selected from the group consisting of:

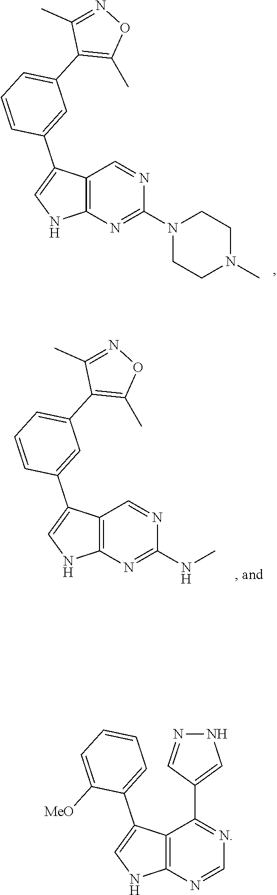 [SMILES: Cc1noc(C)c1-c1cccc(-c2c[nH]c3nc(N4CCN(C)CC4)ncc23)c1 ; CNc1ncc2c(-c3cccc(-c4c(C)noc4C)c3)c[nH]c2n1 ; COc1ccccc1-c1c[nH]c2ncnc(-c3cn[nH]c3)c12]

Some embodiments of the present disclosure include compounds of Formula

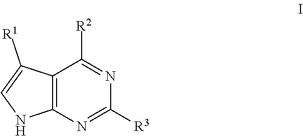

or salts, pharmaceutically acceptable salts, or prodrugs thereof.

Some embodiments of the present disclosure include compounds of Formula Ia:

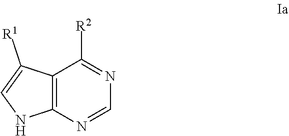

or salts, pharmaceutically acceptable salts, or prodrugs thereof.

Some embodiments of the present disclosure include compounds of Formula Ib:

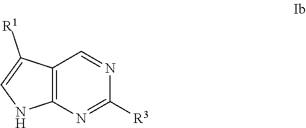

or salts, pharmaceutically acceptable salts, or prodrugs thereof.

Some embodiments of the present disclosure include compounds of Formula Ic:

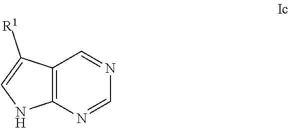

or salts, pharmaceutically acceptable salts, or prodrugs thereof.

In some embodiments of Formulas I, Ia, Ib, and Ic, R¹ is 8-11-membered bicyclic heteroaryl optionally substituted with 1-10 R⁴. IV is 8-11-membered bicyclic heteroaryl optionally substituted with 1-10, 1-9, 1-8, 1-7, 1-6, 1-5, 1-4, 1-3, 1-2, 1) R⁴.

In some embodiments of Formula I, R′ is 9-membered bicyclic heteroaryl optionally substituted with 1-2 R⁴.

In some embodiments of Formula I, R′ is 9-membered bicyclic heteroaryl optionally substituted with one halide (e.g., F, Cl, Br, I) and/or one unsubstituted —(C₁₋₃ alkyl).

In some embodiments of Formula I, R¹ selected from the group consisting of:

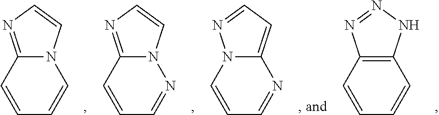

optionally substituted with 1-2 R⁴.

In some embodiments of Formula I, R¹ selected from the group consisting of:

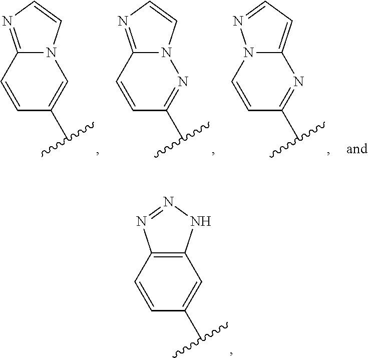 [SMILES: *c1ccc2nccn2c1 ; *c1ccc2nccn2n1 ; *c1ccn2nccc2n1 ; *c1ccc2nn[nH]c2c1]

optionally substituted with 1 R⁴.

In some embodiments of Formula I, R¹ selected from the group consisting of:

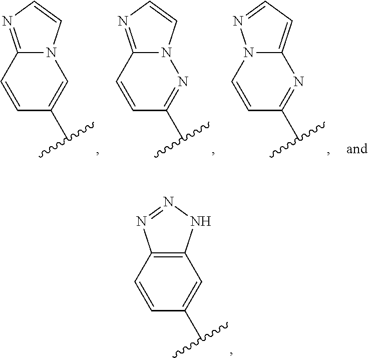 [SMILES: *c1ccc2nccn2c1 ; *c1ccc2nccn2n1 ; *c1ccn2nccc2n1 ; *c1ccc2nn[nH]c2c1]

optionally substituted with one halide or one unsubstituted —(C₁₋₃ alkyl).

In some embodiments of Formulas I and Ib, R¹ selected from the group consisting of:

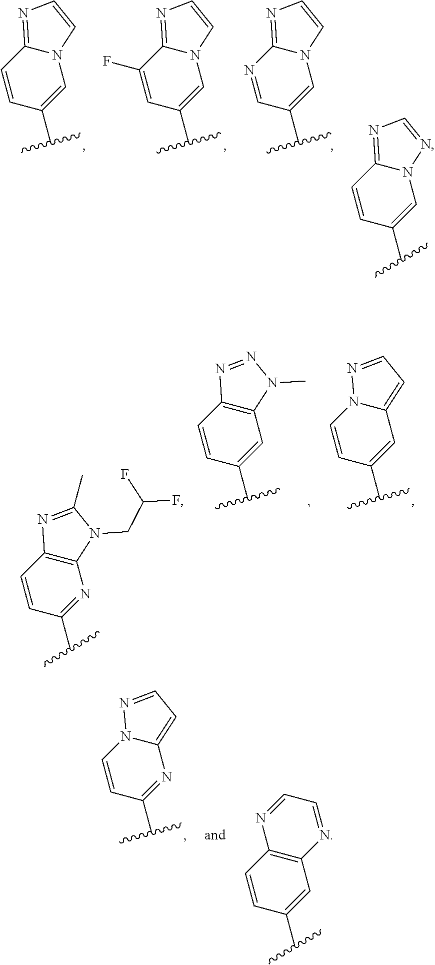

In some embodiments of Formula I, R¹ is selected from the group consisting of 6-membered heteroaryl optionally substituted with 1-2 R⁶ and

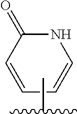

optionally substituted with 1-2 R⁶.

In some embodiments of Formula I, R¹ is selected from the group consisting of pyridine and

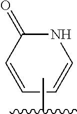

optionally substituted with one —OR^{15a} or one unsubstituted —(C₁₋₃ alkyl).

In some embodiments of Formula I, R¹ is selected from the group consisting of

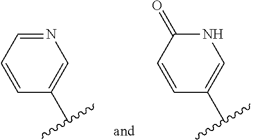 [SMILES: *c1cccnc1 ; *c1ccc(=O)[nH]c1]

optionally substituted with one —OR^{15a} or one unsubstituted —(C₁₋₃ alkyl).

In some embodiments of Formula Ic, R¹ is selected from the group consisting of:

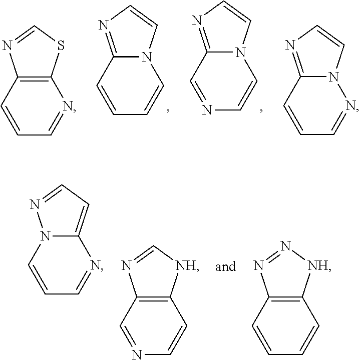

optionally substituted with 1-2 R⁴.

In some embodiments of Formula Ic, R¹ is selected from the group consisting of:

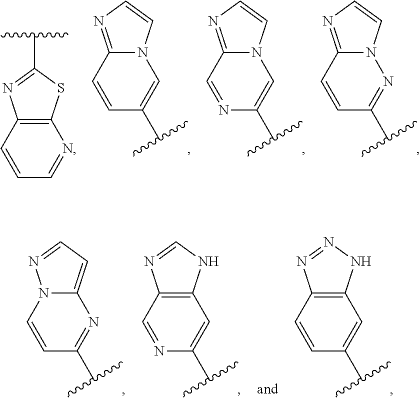

optionally substituted with 1-2 R⁴.

In some embodiments of Formula Ic, R¹ is selected from the group consisting of:

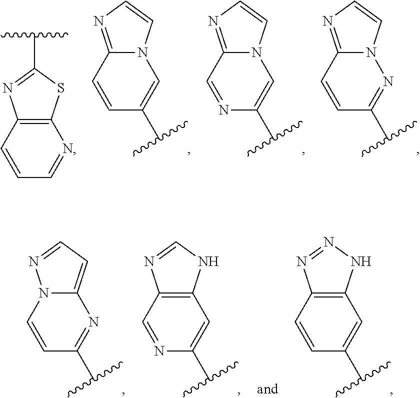

optionally substituted with one halide or one unsubstituted —(C₁₋₃ alkyl).

In some embodiments of Formula I, R¹ is selected from the group consisting of phenyl substituted with 1-5 (e.g., 1-4, 1-3, 1-2, 1) R⁵, 6-membered heteroaryl optionally substituted with 1-4 (e.g., 1-3, 1-2, 1) R⁶, and

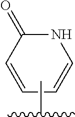

optionally substituted with 1-4 (e.g., 1-3, 1-2, 1) R⁶.

In some embodiments of Formula Ia, R¹ is selected from the heteroaryl group consisting of:

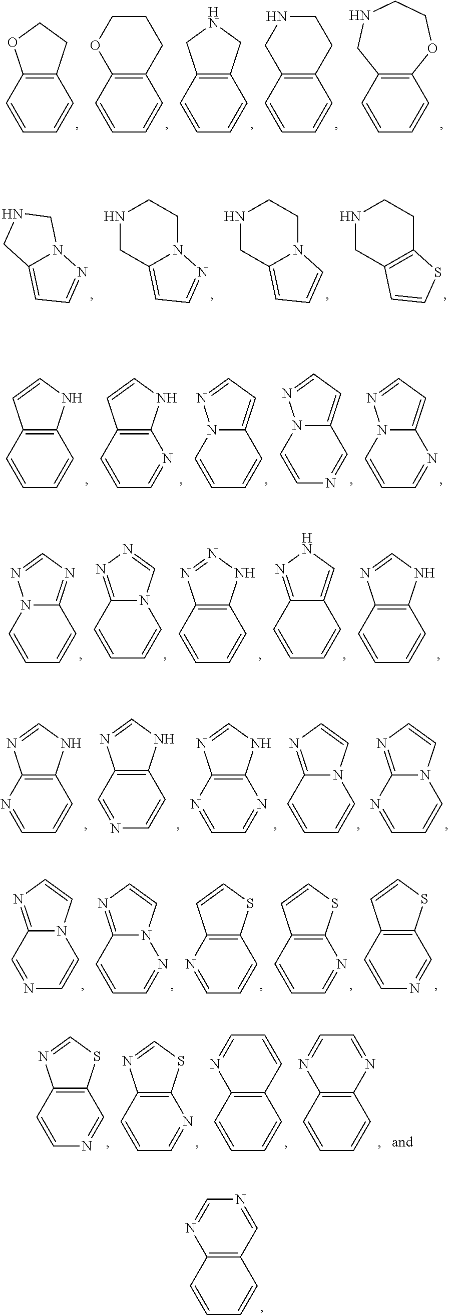

and optionally substituted with 1-10 (e.g., 1-9, 1-8, 1-7, 1-6, 1-5, 1-4, 1-3, 1-2, 1) R⁴.

In some embodiments of Formula Ia, R¹ is selected from the heteroaryl group consisting of:

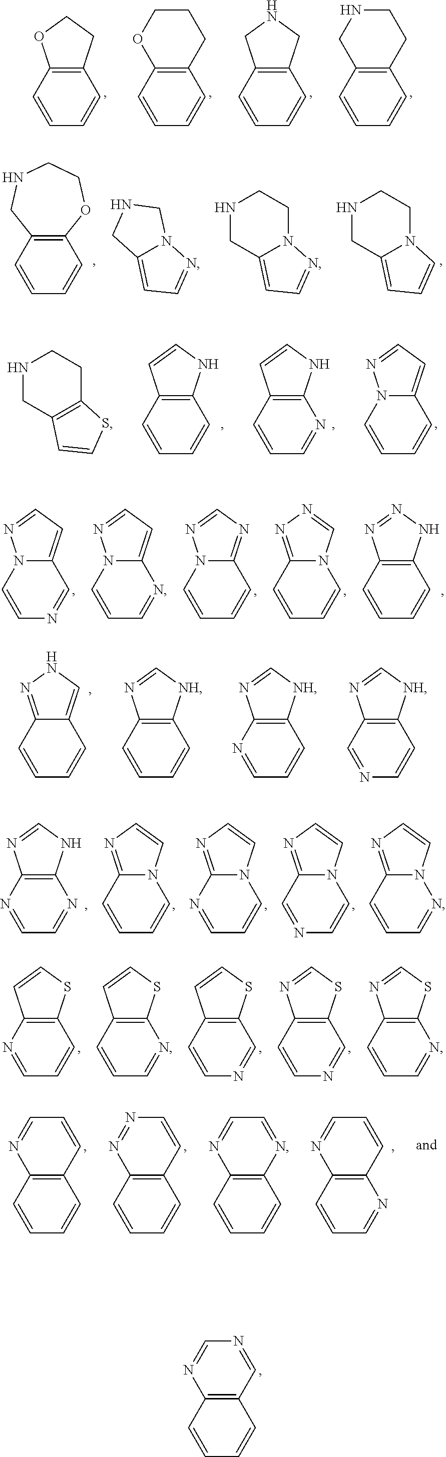

and optionally substituted with 1-10 (e.g., 1-9, 1-8, 1-7, 1-6, 1-5, 1-4, 1-3, 1-2, 1) R⁴.

In some embodiments of Formula Ib, R¹ is selected from the heteroaryl group consisting of:

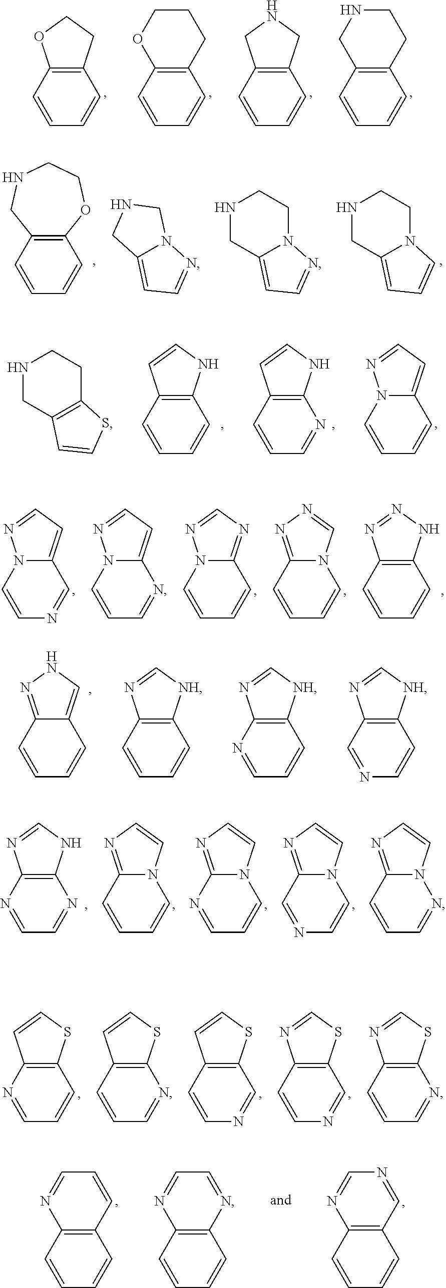

and optionally substituted with 1-10 (e.g., 1-9, 1-8, 1-7, 1-6, 1-5, 1-4, 1-3, 1-2, 1) R⁴.

In some embodiments of Formula Ib, R¹ is selected from the heteroaryl group consisting of:

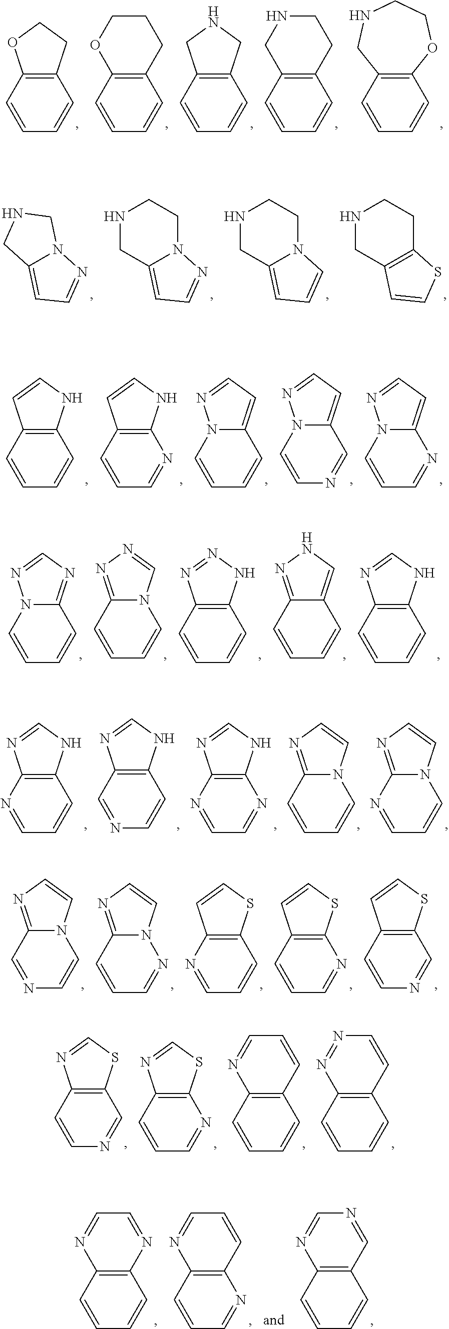

and optionally substituted with 1-10 (e.g., 1-9, 1-8, 1-7, 1-6, 1-5, 1-4, 1-3, 1-2, 1) R⁴.

In some embodiments of Formula Ic, R¹ is selected from the heteroaryl group consisting of:

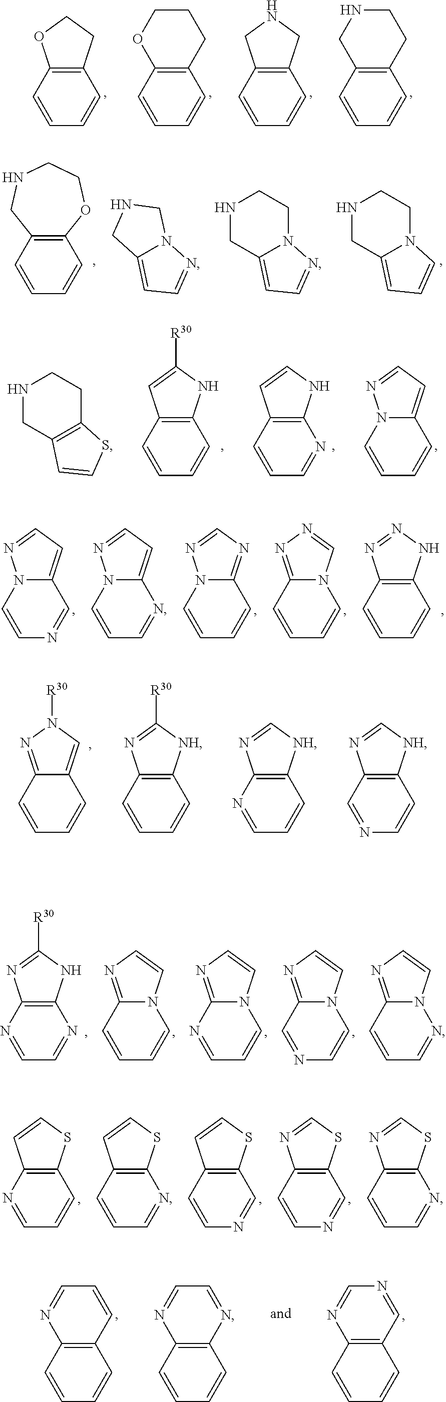

and optionally substituted with 1-10 (e.g., 1-9, 1-8, 1-7, 1-6, 1-5, 1-4, 1-3, 1-2, 1) R⁴.

In some embodiments of Formula Ic, R¹ is selected from the heteroaryl group consisting of:

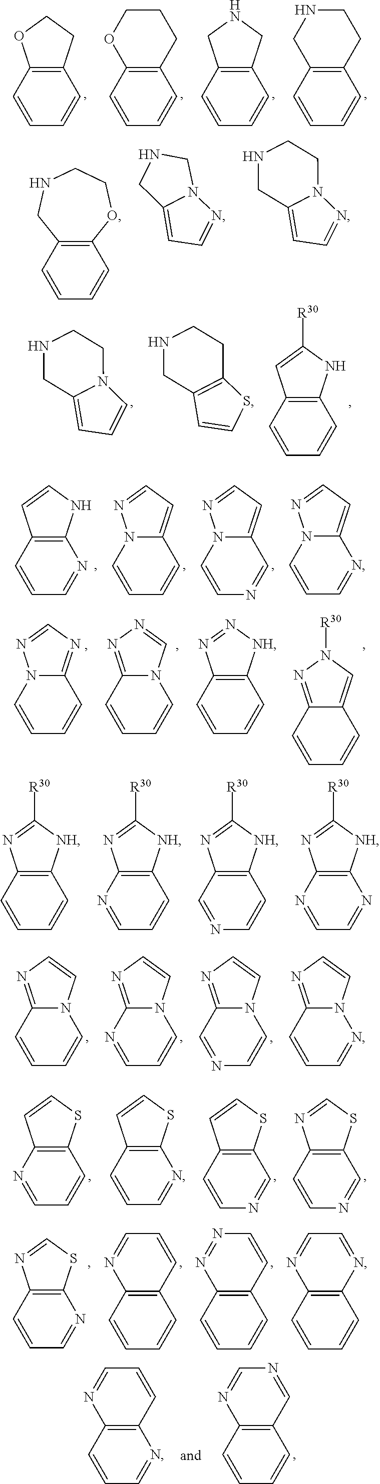

and optionally substituted with 1-10 (e.g., 1-9, 1-8, 1-7, 1-6, 1-5, 1-4, 1-3, 1-2, 1) R⁴.

In some embodiments of Formula I, a carbon atom on an aromatic ring of the heteroaryl form the bond with

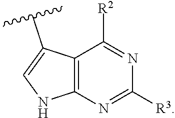

In some embodiments of Formula Ia, a carbon atom on an aromatic ring of the heteroaryl form the bond with

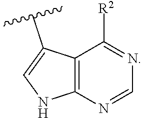

In some embodiments of Formula Ib, a carbon atom on an aromatic ring of the heteroaryl form the bond with

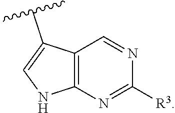

In some embodiments of Formula Ic, a carbon atom on an aromatic ring of the heteroaryl form the bond with

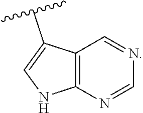 [SMILES: *c1c[nH]c2ncncc12]

In some embodiments of Formulas I and Ia, R² is selected from the group consisting of H, 5-membered heteroaryl optionally substituted with 1-3 (e.g., 1-2, 1) R⁸, and —NHR⁹.

In some embodiments of Formulas I and Ia, R² is selected from the group consisting of —OW, 5-membered heteroaryl optionally substituted with 1-3 (e.g., 1-2, 1) R⁸, and —NHR⁹.

In some embodiments of Formulas I and Ia, R² is H.

In some embodiments of Formulas I and Ia, R² is —OW, in some embodiments of Formulas I and Ia, R² is —OMe, in some embodiments of Formulas I and Ia, R² is —OEt, in some embodiments of Formulas I and Ia, R² is —OW in some embodiments of Formulas I and Ia, R² is —OⁱPr, in some embodiments of Formulas I and Ia, R² is —OCH₂CH₂OH, and in some embodiments of Formulas I and Ia, R² is —OCH₂CH₂OMe.

In some embodiments of Formulas I and Ia, R² is —NHR⁹, in some embodiments of Formulas I and Ia, R² is —NHMe, in some embodiments of Formulas I and Ia, R² is —NHEt, in some embodiments of Formulas I and Ia, R² is —WTI⁻, in some embodiments of Formulas I and Ia, R² is —NH′Pr.

In some embodiments of Formulas I and Ia, when R² is —NHR⁹, R¹ is selected from the group consisting of:

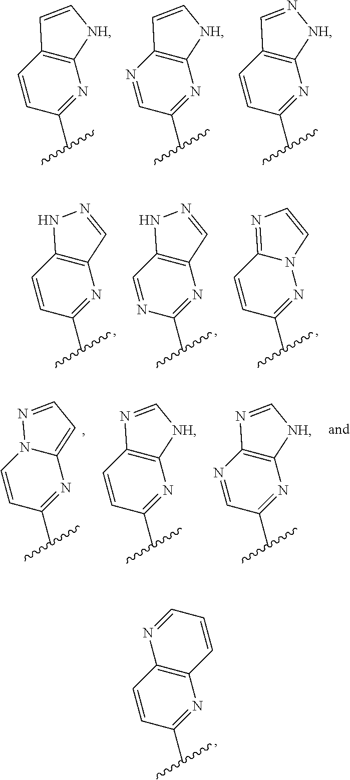

and optionally substituted with 1-3 R⁴.

In some embodiments of Formulas I and Ia, when R² is —NHR⁹, R¹ is selected from the group consisting of:

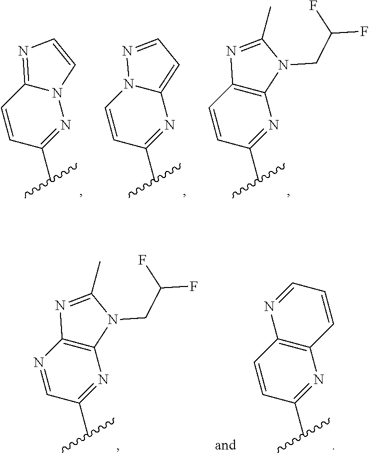

In some embodiments of Formulas I and Ia, when R² is —NHMe, R¹ is selected from the group consisting of:

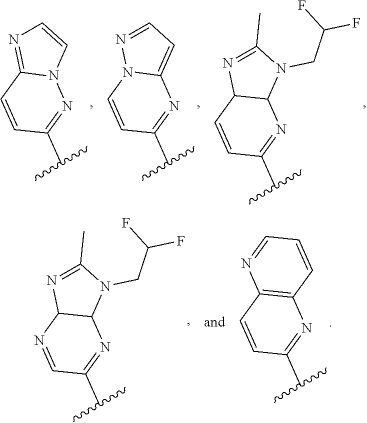

In some embodiments of Formulas I and Ib, R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 (e.g., 1-9, 1-8, 1-7, 1-6, 1-5, 1-4, 1-3, 1-2, 1) R¹¹, -(5-10 membered heteroaryl) optionally substituted with 1-10 (e.g., 1-9, 1-8, 1-7, 1-6, 1-5, 1-4, 1-3, 1-2, 1) R¹², —(CH₂)ₚcarbocyclyl optionally substituted with 1-12 (e.g., 1-11, 1-10, 1-9, 1-8, 1-7, 1-6, 1-5, 1-4, 1-3, 1-2, 1) R¹³, and —NHR¹⁴.

In some embodiments of Formulas I and Ib, R³ is selected from the group consisting of H, —OR¹⁰, unsubstituted —(C₁₋₅ alkyl), unsubstituted —(C₁₋₅ haloalkyl), -heterocyclyl optionally substituted with 1-2 R¹¹, -(5-10 membered heteroaryl) optionally substituted with 1-2 R¹², (CH₂)_{pcar}b_{ocyc}l_{yl opt}iₒₙₐll_{y} substituted with 1-3 R¹³, and —NHR¹⁴.

In some embodiments of Formulas I and Ib, R³ is selected from the group consisting of -carbocyclyl optionally substituted with 1-2 R¹³ and —NHR¹⁴.

In some embodiments of Formulas I and Ib, R³ is selected from the group consisting of unsubstituted —(C1-5 alkyl) and —NHR¹⁴

In some embodiments of Formulas I and Ib, R³ is —NHR¹⁴.

In some embodiments of Formulas I and Ib, R³ is selected from the group

consisting of:

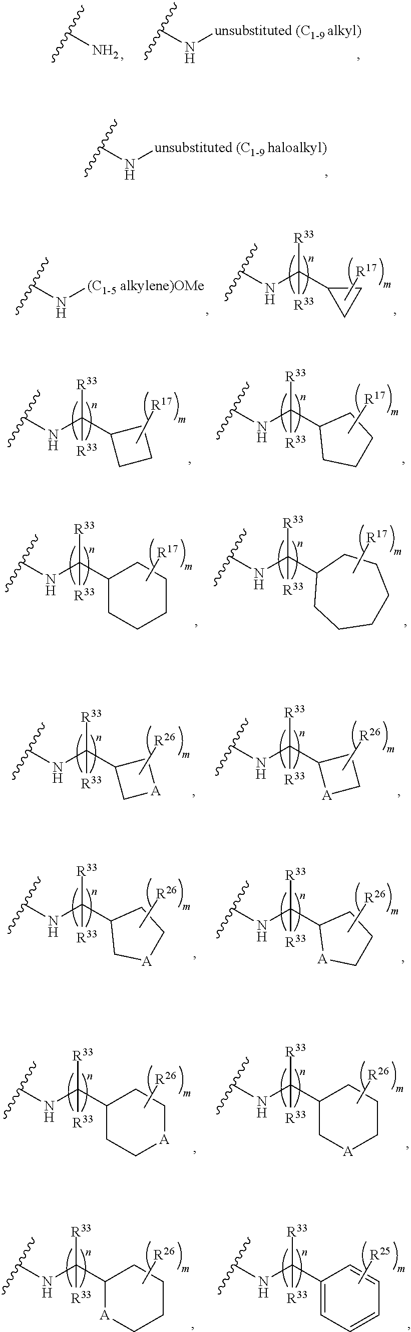

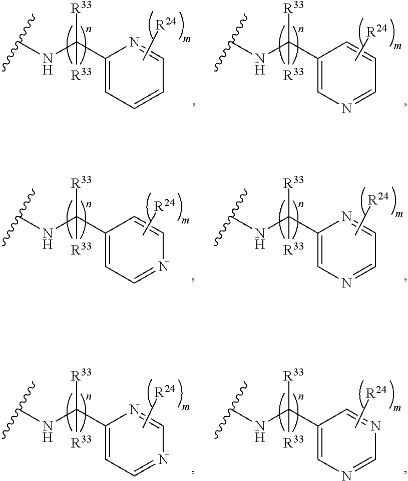

wherein n is 0-3; m is 0-6;

A is selected from the group consisting of N, O, and S; and R³³ is independently selected from the group consisting of H, halide, CH₃, and CF₃.

In some embodiments of Formulas I and Ib, R³ is selected from the group consisting of:

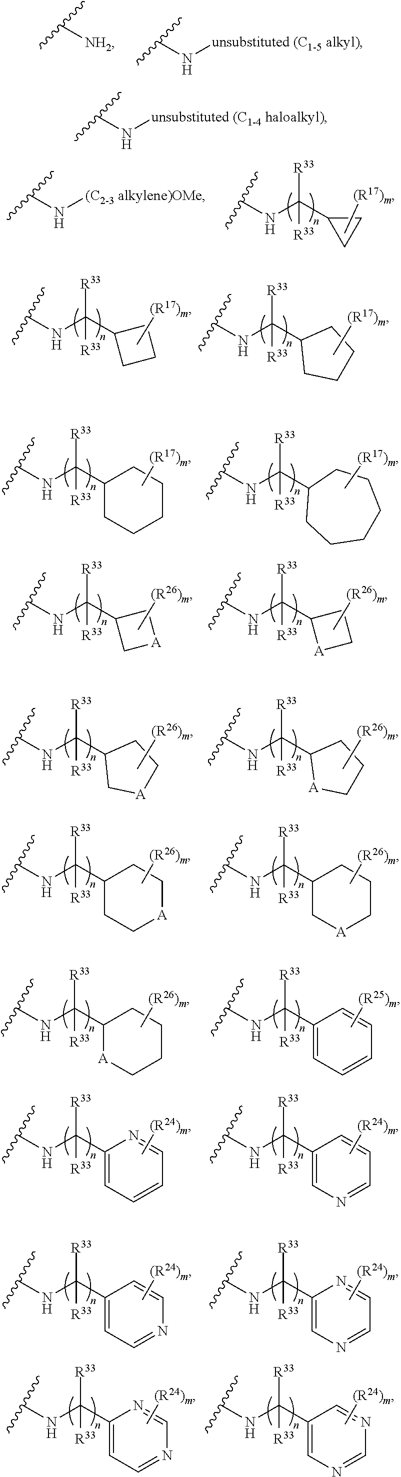

wherein n is 0-1; m is 0-2; A is selected from the group consisting of N and 0; and R³³ is independently selected from the group consisting of H, F, CH₃, and CF₃.

In some embodiments of Formulas I and Ib, R³ is selected from the group consisting of:

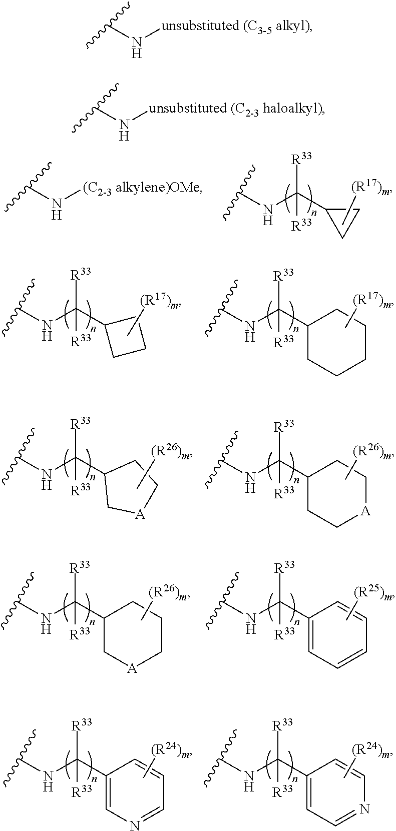

wherein n is 0-1; m is 0-2;

A is selected from the group consisting of N and 0; and each R¹' is independently selected from the group consisting of F, —OR³¹, unsubstituted —(C₁₋₂ alkyl), and unsubstituted —(C₁₋₂haloalkyl); each R²⁴ is independently selected from the group consisting of F, CH₃, CF3, and -heterocyclyl optionally substituted with 1-2 R¹⁵; each R²⁵ is independently selected from the group consisting of F, CH₃, CF3, and -heterocyclyl optionally substituted with 1-2 R¹⁵; each R²⁶ is independently selected from the group consisting of F, —OH, —OMe, CH₃, and CF3; each R³³ is independently selected from the group consisting of H, F, CH₃, and CF₃.

In some embodiments of Formulas I and Ib, R³ is selected from the group consisting of:

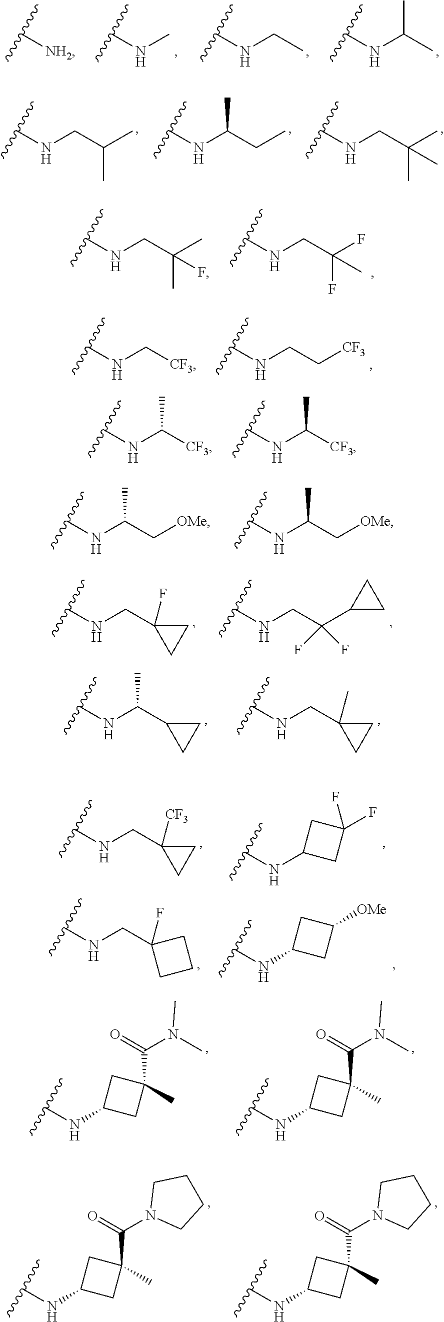

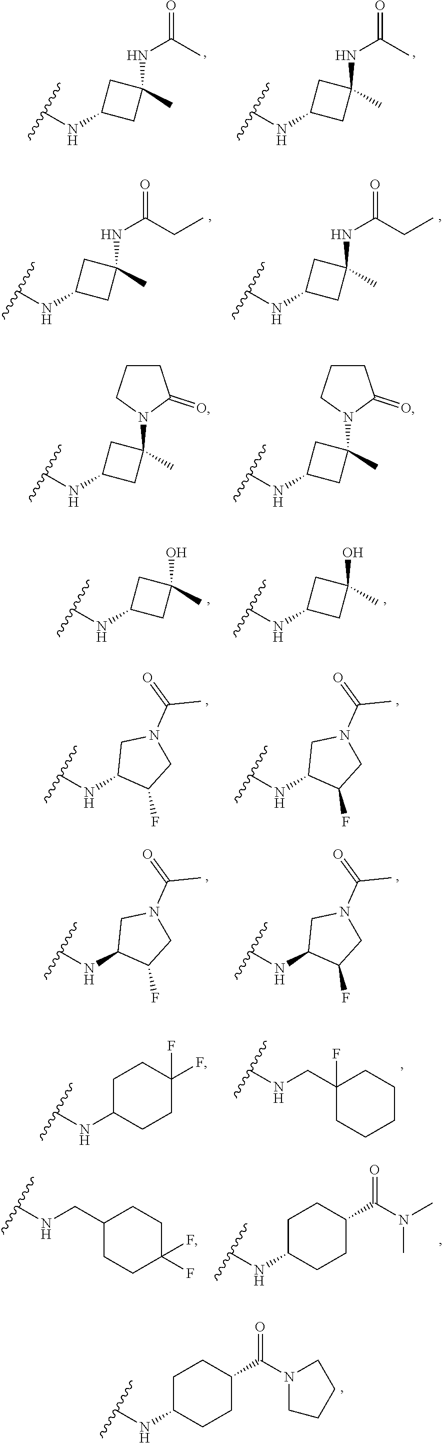

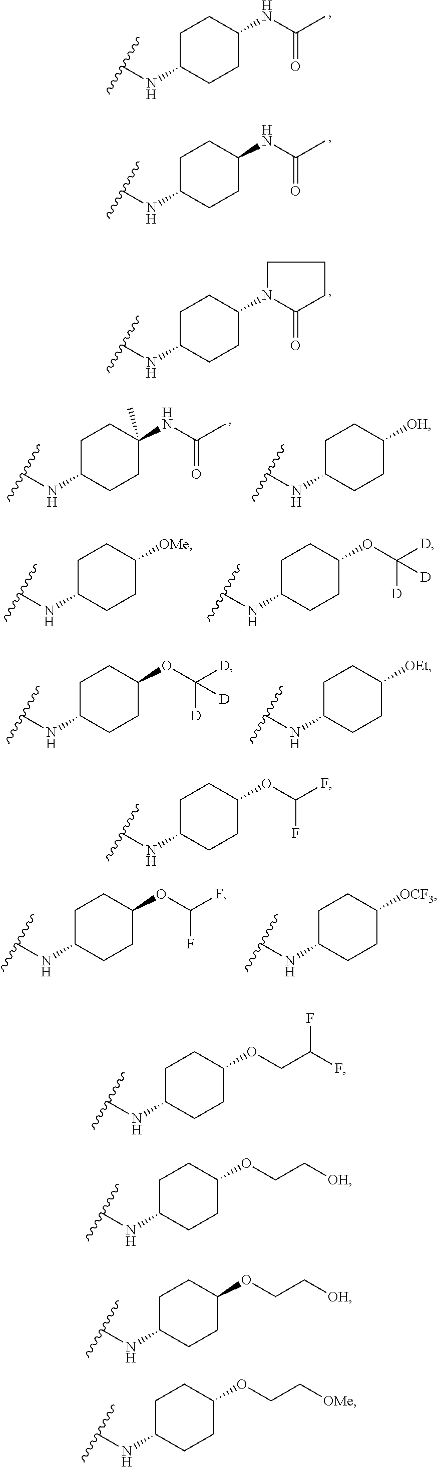

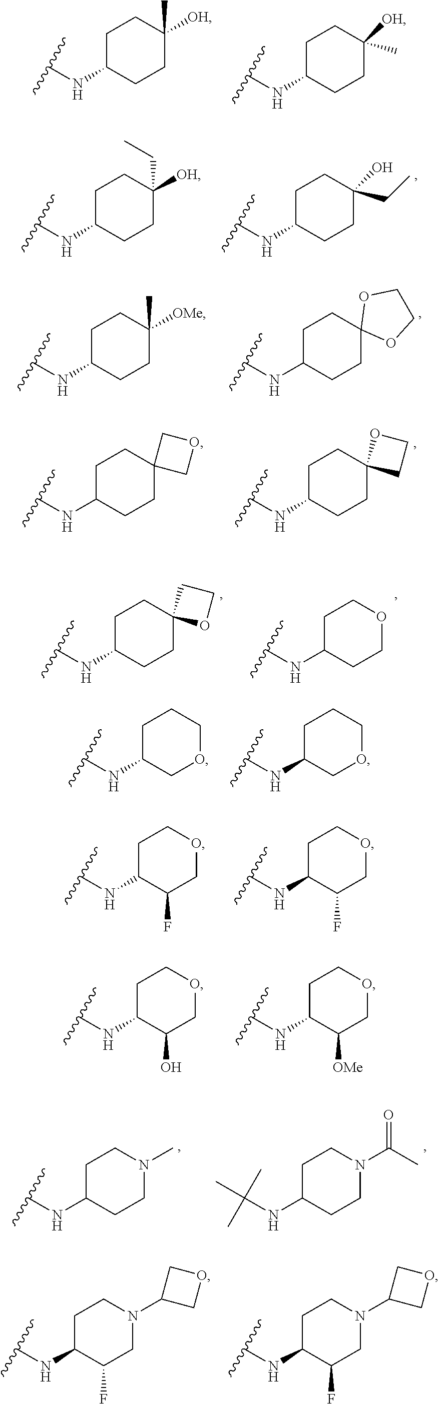

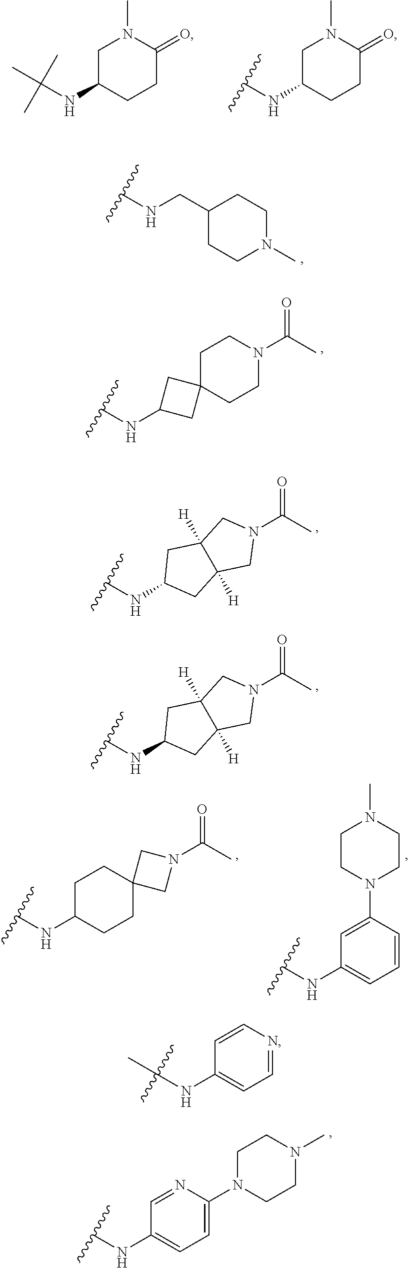

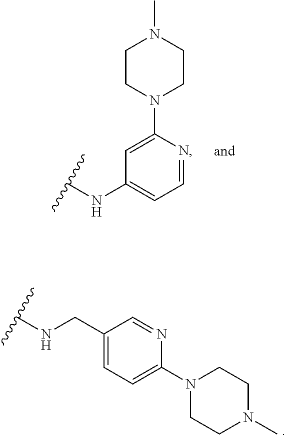 [SMILES: *Nc1ccnc(N2CCN(C)CC2)c1 ; "and" ; *NCc1ccc(N2CCN(C)CC2)nc1]

In some embodiments of Formulas I and Ib, R³ is selected from the group consisting of:

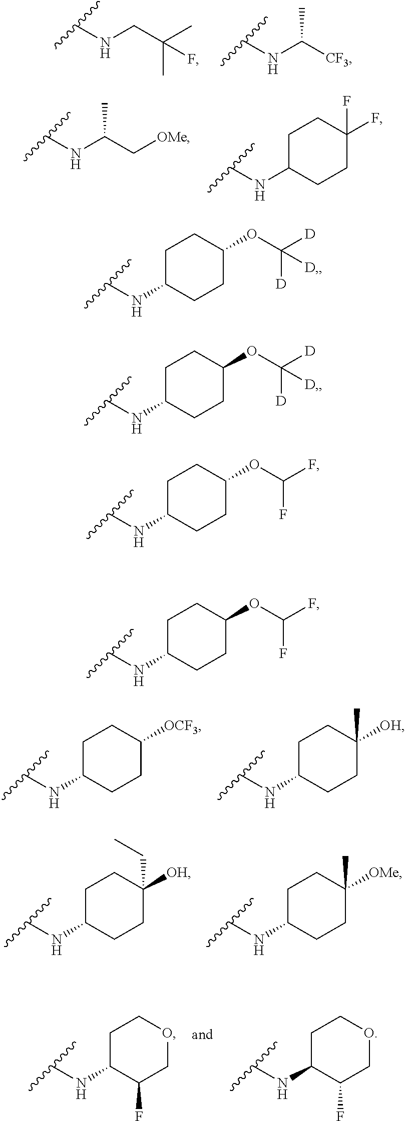

In some embodiments of Formulas I and Ib, R³ is selected from the group consisting of:

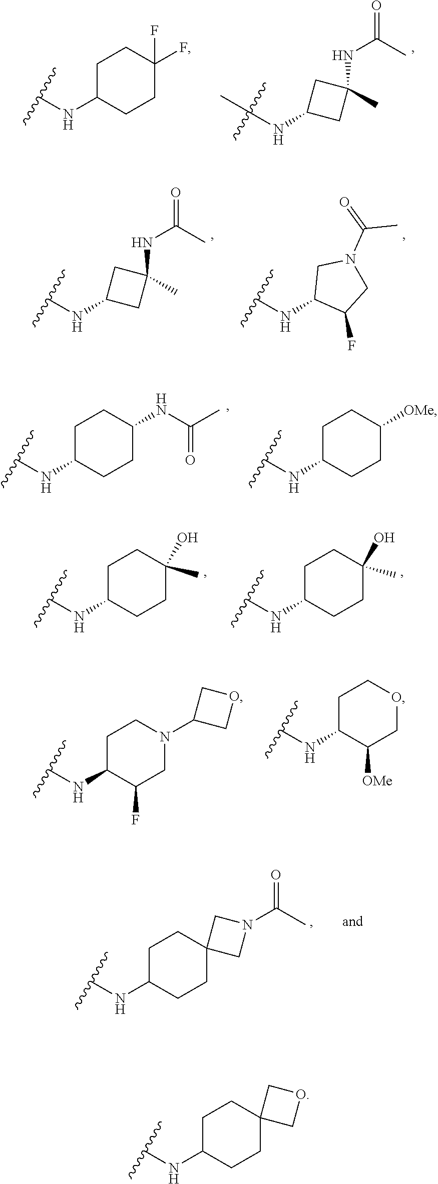

In some embodiments of Formulas I and Ib, there is the proviso that R² and R³ are not both H.

In some embodiments of Formulas I and Ib, there is the proviso that either R² or R³ is H but R² and R³ are not both H.

In some embodiments of Formulas I, Ia, Ib, and Ic, each R⁴ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 (e.g., 1-9, 1-8, 1-7, 1-6, 1-5, 1-4, 1-3, 1-2, 1) R¹⁶, -heteroaryl optionally substituted with 1-10 (e.g., 1-9, 1-8, 1-7, 1-6, 1-5, 1-4, 1-3, 1-2, 1) R¹⁷, -carbocyclyl optionally substituted with 1-12 (e.g., 1-11, 1-10, 1-9, 1-8, 1-7, 1-6, 1-5, 1-4, 1-3, 1-2, 1) R⁸, —C(═O)N(R¹⁹)2, and —C(═O)R²⁰.

In some embodiments of Formulas I, Ia, Ib, and Ic, each R⁴ is independently selected from the group consisting of F, Cl, unsubstituted —(C₁₋₃ alkyl), unsubstituted —(C₁₋₃ haloalkyl), —OR¹⁵, —CH₂OH, -heterocyclyl optionally substituted with 1-2 R¹⁶, -heteroaryl optionally substituted with 1-2 R¹⁷, -carbocyclyl optionally substituted with 1-2 R⁸, —C(═O)N(R¹⁹)2, and —C(═O)R²⁰.

In some embodiments of Formulas I, Ia, Ib, and Ic, each R⁴ is independently selected from the group consisting of F, Cl, unsubstituted —(C₁₋₃ alkyl), unsubstituted —(C₁₋₃ haloalkyl), —OMe, —OCHF2, —OCF₃, and —CH₂OH.

In some embodiments of Formulas I, Ia, Ib, and Ic, two R⁴ attached to the same carbon atom are taken together to form a carbonyl group.

In some embodiments of Formula I, each R⁵ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 (e.g., 1-9, 1-8, 1-7, 1-6, 1-5, 1-4, 1-3, 1-2, 1) R¹⁶, -heteroaryl optionally substituted with 1-10 (e.g., 1-9, 1-8, 1-7, 1-6, 1-5, 1-4, 1-3, 1-2, 1) R¹⁷, -carbocyclyl optionally substituted with 1-12 (e.g., 1-11, 1-10, 1-9, 1-8, 1-7, 1-6, 1-5, 1-4, 1-3, 1-2, 1) R⁸, —C(═O)N(R¹⁹)2, and —C(═O)R²⁰.

In some embodiments of Formula I, each R⁶ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 (e.g., 1-9, 1-8, 1-7, 1-6, 1-5, 1-4, 1-3, 1-2, 1) R^{16a}, -heteroaryl optionally substituted with 1-10 (e.g., 1-9, 1-8, 1-7, 1-6, 1-5, 1-4, 1-3, 1-2, 1) R^{17a}, -carbocyclyl optionally substituted with 1-12 (e.g., 1-11, 1-10, 1-9, 1-8, 1-7, 1-6, 1-5, 1-4, 1-3, 1-2, 1) R^{18a}, —C(═O)N(R¹⁹a)₂, and —C(═O)R²″.

In some embodiments of Formulas I and Ia, R⁷ is selected from the group consisting of unsubstituted —(C₁₋₉ alkyl), unsubstituted —(C₂₋₉ alkenyl), unsubstituted —(C₂₋₉-₉ alkynyl), unsubstituted —(C₁₋₉ haloalkyl), and —(CH₂)heterocyclyl optionally substituted with 1-10 (e.g., 1-9, 1-8, 1-7, 1-6, 1-5, 1-4, 1-3, 1-2, 1) R²¹.

In some embodiments of Formulas I and Ia, R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹.

In some embodiments of Formulas I and Ia, R⁷ is selected from the group consisting of unsubstituted —(C₁₋₃ alkyl), unsubstituted —(C₁₋₃ haloalkyl), and —CH₂CH₂OR³².

In some embodiments of Formulas I and Ia, R⁷ is selected from the group consisting of unsubstituted —(C₁₋₂ alkyl), unsubstituted —(C₁₋₂ haloalkyl), —CH₂CH₂OH, and —CH₂CH₂₀Me.

In some embodiments of Formulas I and Ia, each R⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ia, R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 (e.g., 1-11, 1-10, 1-9, 1-8, 1-7, 1-6, 1-5, 1-4, 1-3, 1-2, 1) R²², and -heterocyclyl optionally substituted with 1-10 (e.g., 1-9, 1-8, 1-7, 1-6, 1-5, 1-4, 1-3, 1-2, 1) R²³.

In some embodiments of Formulas I and Ia, R⁹ is selected from the group consisting of unsubstituted —(C₁₋₂ alkyl) and unsubstituted —(C₁₋₂ haloalkyl).

In some embodiments of Formulas I and Ib, R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ia, R¹⁰ is selected from the group consisting of unsubstituted —(C₁₋₃ alkyl) and unsubstituted —(C₁₋₃ haloalkyl).

In some embodiments of Formulas I and Ib, each R¹¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ib, each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³².

In some embodiments of Formulas I and Ib, each R¹² is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C ₁₋₉ haloalkyl).

In some embodiments of Formulas I and Ib, each R¹³ is independently selected from the group consisting of H, halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ib, R^{1A} is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₃ alkylene)OMe, —(C₁₋₃ alkylene)ₚcarbocyclyl optionally substituted with 1-12 (e.g., 1-11, 1-10, 1-9, 1-8, 1-7, 1-6, 1-5, 1-4, 1-3, 1-2, 1) R¹⁷, —(C₁₋₉ alkylene)ₚheteroaryl optionally substituted with 1-10 (e.g., 1-9, 1-8, 1-7, 1-6, 1-5, 1-4, 1-3, 1-2, 1) R²⁴, (C₁₋₃ alkylene)ₚphenyl optionally substituted with 1-10 R²⁵, and —(C₁₋₃ alkylene)ₚheterocyclyl optionally substituted with 1-10 (e.g., 1-9, 1-8, 1-7, 1-6, 1-5, 1-4, 1-3, 1-2, 1) R²⁶, wherein each —(C₁₋₃ alkylene) is, independently, optionally substituted with 1-5 (e.g., 1-4, 1-3, 1-2, 1) halide (e.g., F, Cl, Br, I) and/or 1-5 (e.g., 1-4, 1-3, 1-2, 1) unsubstituted —(C₁₋₃ alkyl).

In some embodiments of Formulas I and Ib, R^{1A} is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C ₁₋₃ alkylene) is, independently, optionally substituted with 1-5 halide and/or 1-5 unsubstituted —(C₁₋₃ alkyl).

In some embodiments of Formulas I and Ib, R^{1A} is selected from the group consisting of H, unsubstituted —(Cl_s alkyl), unsubstituted —(Cl_shaloalkyl), —(C₂₋₃ alkylene)OMe, —(CR³⁸)ₚcarbocyclyl optionally substituted with 1-3 R¹⁷, —(CR³⁸)ₚheteroaryl optionally substituted with 1-2 R²⁴, —(CR³⁸)ₚphenyl optionally substituted with 1-10 R²⁵, and —(CR³⁸)ₚheterocyclyl optionally substituted with 1-2 R²⁶.

In some embodiments of Formulas I and Ib, R^{1A} is selected from the group consisting of unsubstituted —(C1-5 alkyl), unsubstituted —(C1.5 haloalkyl), —(C₂₋₃alkylene)OMe, and -(CR³⁸)ₚcarbocyclyl optionally substituted with 1-3 R¹⁷.

In some embodiments of Formulas I and Ib, R^{1A} is selected from the group consisting of —(C₁₋₃ alkylene)ₚcarbocyclyl optionally substituted with 1-12 (e.g., 1-11, 1-10, 1-9, 1-8, 1-7, 1-6, 1-5, 1-4, 1-3, 1-2, 1) R¹⁷ and —(C₁₋₃ alkylene)ₚheterocyclyl optionally substituted with 1-10 (e.g., 1-9, 1-8, 1-7, 1-6, 1-5, 1-4, 1-3, 1-2, 1) R²⁶, wherein each —(C₁₋₃ alkylene) is, independently, optionally substituted with 1-5 (e.g., 1-4, 1-3, 1-2, 1) halide (e.g., F, Cl, Br, I) and/or 1-5 (e.g., 1-4, 1-3, 1-2, 1) unsubstituted —(C₁₋₃ alkyl).

In some embodiments of Formulas I and Ib, R^{1A} is selected from the group consisting of -carbocyclyl optionally substituted with 1-5 (e.g., 1-4, 1-3, 1-2, 1) R¹⁷ and -heterocyclyl optionally substituted with 1-5 (e.g., 1-4, 1-3, 1-2, 1) R²⁶.

In some embodiments of Formulas I, Ia, Ib, and Ic, each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R¹⁶.

In some embodiments of Formula I, each R^{15a} is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R¹⁶a.

In some embodiments of Formulas I, Ia, Ib, and Ic, each R¹⁶ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R^{16a} is independently selected from the group consisting of halide (e.g., F, Cl, Br, I), unsubstituted —(C₁₋₉ alkyl), unsubstituted —(C₂₋₉-₉ alkenyl), unsubstituted —(C₂₋₉ alkynyl), and unsubstituted —(C₁₋₉ haloalkyl).

In some embodiments of Formulas I, Ia, Ib, and Ic, each R¹⁷ is independently selected from the group consisting of halide (e.g., F, Cl, Br, I), —OR³¹, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R¹⁷ is independently selected from the group consisting of F, —OR³¹, unsubstituted —(C1-2 alkyl), and unsubstituted —(C₁₋₉ 2 haloalkyl).

In some embodiments of Formulas I, Ia, Ib, and Ic, each R¹⁷ is independently selected from the group consisting of halide, -01V¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵.

In some embodiments of Formula I, each R¹⁷′ is independently selected from the group consisting of halide (e.g., F, Cl, Br, I), -OMe, unsubstituted —(C₁₋₉ alkyl), unsubstituted -(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R¹⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R¹⁸′ is independently selected from the group consisting of halide (e.g., F, Cl, Br, I), unsubstituted —(C₁₋₉ alkyl), unsubstituted —(C₂₋₉-₉ alkenyl), unsubstituted —(C₂₋₉ alkynyl), and unsubstituted —(C₁₋₉ haloalkyl).

In some embodiments of Formulas I, Ia, Ib, and Ic, each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 (e.g., 1-11, 1-10, 1-9, 1-8, 1-7, 1-6, 1-5, 1-4, 1-3, 1-2, 1) R²⁷, -heterocyclyl optionally substituted with 1-10 (e.g., 1-9, 1-8, 1-7, 1-6, 1-5, 1-4, 1-3, 1-2, 1) R²⁸, and -heteroaryl optionally substituted with 1-10 (e.g., 1-9, 1-8, 1-7, 1-6, 1-5, 1-4, 1-3, 1-2, 1) R²⁹.

In some embodiments of Formula I, each R¹⁹′ are independently selected from the group consisting of H, unsubstituted —(C₁₋₉ alkyl), unsubstituted —(C₂₋₉ alkenyl), unsubstituted -(C₂₋₉ alkynyl), and unsubstituted —(C₁₋₉ haloalkyl); —(CH₂)ₚcarbocyclyl optionally substituted with 1-12 (e.g., 1-11, 1-10, 1-9, 1-8, 1-7, 1-6, 1-5, 1-4, 1-3, 1-2, 1) R^{27a}, -heterocyclyl optionally substituted with 1-10 (e.g., 1-9, 1-8, 1-7, 1-6, 1-5, 1-4, 1-3, 1-2, 1) R^{28a}, and -heteroaryl optionally substituted with 1-10 (e.g., 1-9, 1-8, 1-7, 1-6, 1-5, 1-4, 1-3, 1-2, 1) R^{29a}

In some embodiments of Formulas I, Ia, Ib, and Ic, each R²⁰ is -heterocyclyl optionally substituted with 1-10 (e.g., 1-9, 1-8, 1-7, 1-6, 1-5, 1-4, 1-3, 1-2, 1) R²⁸.

In some embodiments of Formula I, each R²⁰′ is -heterocyclyl optionally substituted with 1-10 (e.g., 1-9, 1-8, 1-7, 1-6, 1-5, 1-4, 1-3, 1-2, 1) R²⁸a.

In some embodiments of Formulas I and Ia, each R²¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ia, each R²² is independently selected from the group consisting of halide (e.g., F, Cl, Br, I), -OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ia, each R²³ is independently selected from the group consisting of halide (e.g., F, Cl, Br, I), -OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R¹⁶.

In some embodiments of Formulas I and Ib, each R²⁴ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R¹⁶.

In some embodiments of Formulas I and Ib, each R²⁴ is independently selected from the group consisting of F, CH₃, CF3, and -heterocyclyl optionally substituted with 1-2 R¹⁵.

In some embodiments of Formulas I and Ib, each R²⁵ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R¹⁶.

In some embodiments of Formulas I and Ib, each R²⁵ is independently selected from the group consisting of F, CH₃, CF3, and -heterocyclyl optionally substituted with 1-2 R¹⁵.

In some embodiments of Formulas I and Ib, each R²⁶ is independently selected from the group consisting of halide (e.g., F, Cl, Br, I), -OMe, -SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ib, each R²⁶ is independently selected from the group consisting of halide, —OR³², —SO₂Me, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ib, each R²⁶ is independently selected from the group consisting of halide, —OR³², —C(═O)R³⁶, -SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸.

In some embodiments of Formulas I and Ib, two R²⁶ attached to the same carbon atom are taken together to form a carbonyl group.

In some embodiments of Formulas I and Ib, each R²⁶ is independently selected from the group consisting of F, —OH, —OMe, CH₃, and CF3.

In some embodiments of Formulas I, Ia, Ib, and Ic, each R²⁷ is independently selected from the group consisting of halide (e.g., F, Cl, Br, I), —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R^{27a} is independently selected from the group consisting of halide (e.g., F, Cl, Br, I), —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R²⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C ₁₋₉ haloalkyl).

In some embodiments of Formula I, each R^{28a} is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R²⁹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas Ic, each R³° is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R³¹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³².

In some embodiments of Formulas I, Ia, Ib, and Ic, each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, and Ic, each R³⁴ is independently selected from the group consisting of—N(R³⁷)₂, and -heterocyclyl optionally substituted with 1-10 (e.g., 1-9, 1-8, 1-7, 1-6, 1-5, 1-4, 1-3, 1-2, 1) R²⁸.

In some embodiments of Formulas I, Ia, Ib, and Ic, each R³⁵ is independently selected from the group consisting of halide (e.g., F, Cl, Br, I), unsubstituted —(C₁₋₉ alkyl), unsubstituted —(C₂₋₉ alkenyl), unsubstituted —(C₂₋₉alkynyl), and unsubstituted —(C ₁₋₉ haloalkyl).

In some embodiments of Formulas I, Ia, Ib, and Ic, each two R³⁵ attached to the same carbon atom are taken together to form a carbonyl group.

In some embodiments of Formulas I, Ia, Ib, and Ic, each R³⁶ is independently selected from the group consisting of unsubstituted —(C₁₋₉ alkyl), unsubstituted —(C₂₋₉ alkenyl), unsubstituted —(C₂₋₉alkynyl), and unsubstituted —(C₁₋₉ haloalkyl).

In some embodiments of Formulas I, Ia, Ib, and Ic, each R³⁷ is independently selected from the group consisting of H, unsubstituted —(C₁₋₉ alkyl), unsubstituted —(C₂₋₉ alkenyl), unsubstituted —(C₂₋₉alkynyl), and unsubstituted —(C₁₋₉ haloalkyl).

In some embodiments of Formulas I and Ib, each R³⁸ is independently selected from the group consisting of H, halide, CH₃, and CF3.

In some embodiments of Formulas I and Ib, each R³⁸ is independently selected from the group consisting of H, F, CH₃, and CF3.

In some embodiments of Formulas I and Ib, A is selected from the group consisting of N, O, and S.

In some embodiments of Formulas I and Ib, A is selected from the group consisting of N and O.

In some embodiments of Formulas I and Ib, each n is independently 0-3.

In some embodiments of Formulas I and Ib, each m is independently 0-6.

In some embodiments of Formulas I, Ia, Ib, and Ic, each p is independently 0 or 1.

In some embodiments of Formulas I, Ia, Ib, and Ic, each H atom is optionally independently replaced by ²H (D) (deuterium).

In some embodiments of Formulas I, there is the proviso that Formula I is not a structure selected from the group consisting of:

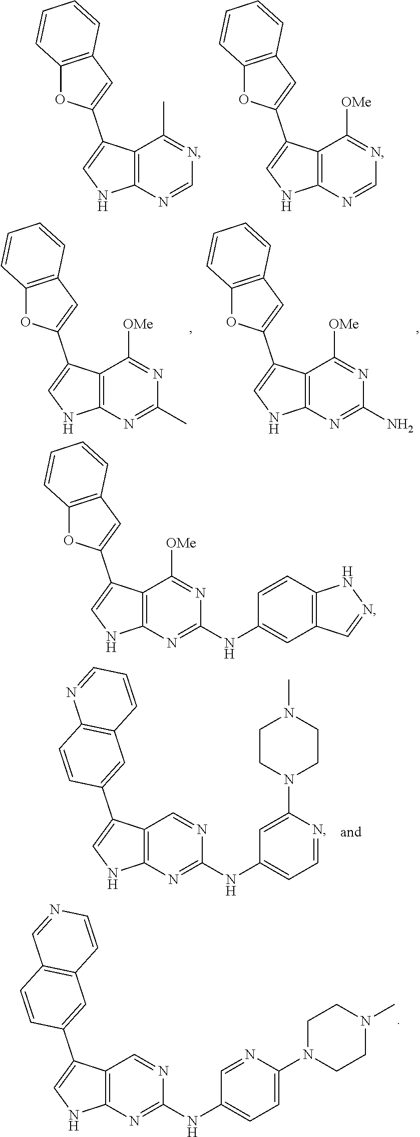

In some embodiments of Formulas I, there is the proviso that Formula I is not a structure selected from the group consisting of:

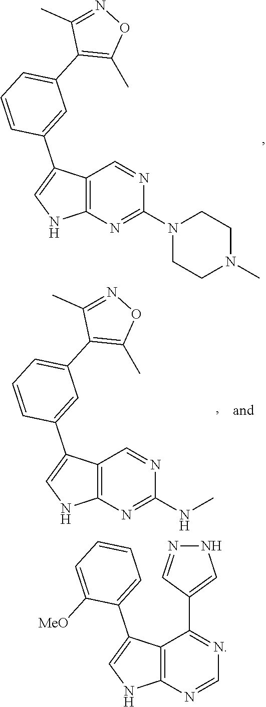

In some embodiments of Formulas Ia, there is the proviso that Formula Ia is not a structure selected from the group consisting of:

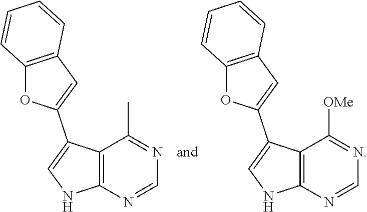 [SMILES: Cc1ncnc2[nH]cc(-c3cc4ccccc4o3)c12 ; COc1ncnc2[nH]cc(-c3cc4ccccc4o3)c12]

In some embodiments of Formulas Ia, there is the proviso that Formula Ia is not a structure of:

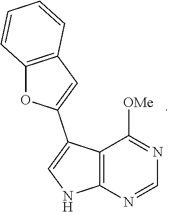 [SMILES: COc1ncnc2[nH]cc(-c3cc4ccccc4o3)c12]

In some embodiments of Formulas Ib, there is the proviso that Formula Ib is not a structure selected from the group consisting of:

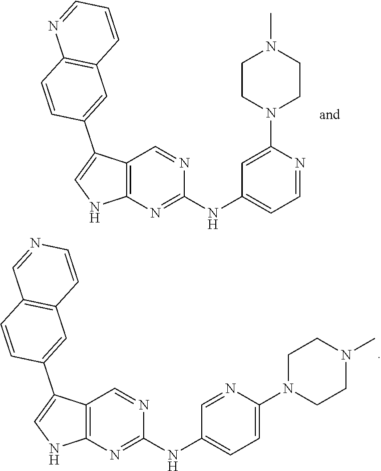 [SMILES: CN1CCN(c2cc(Nc3ncc4c(-c5ccc6ncccc6c5)c[nH]c4n3)ccn2)CC1 ; CN1CCN(c2ccc(Nc3ncc4c(-c5ccc6cnccc6c5)c[nH]c4n3)cn2)CC1]

In some embodiments of Formulas Ib, there is the proviso that Formula Ib is not a structure selected from the group consisting of:

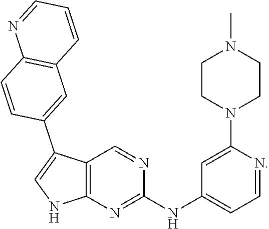 [SMILES: CN1CCN(c2cc(Nc3ncc4c(-c5ccc6ncccc6c5)c[nH]c4n3)ccn2)CC1]

Illustrative compounds of Formulas I, Ia, Ib, and Ic, are shown in Table 1.

TABLE 1

| | |
|---|---|
| 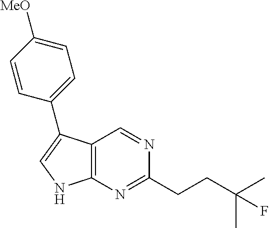 [SMILES: COc1ccc(-c2c[nH]c3nc(CCC(C)(C)F)ncc23)cc1] | 1 |
| 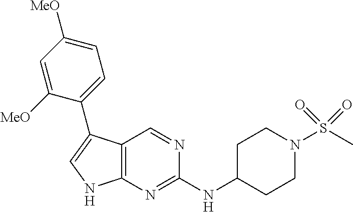 [SMILES: COc1ccc(-c2c[nH]c3nc(NC4CCN(S(C)(=O)=O)CC4)ncc23)c(OC)c1] | 2 |
| 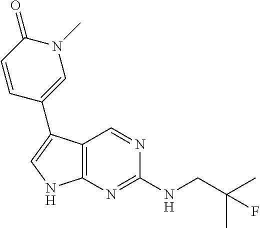 [SMILES: Cn1cc(-c2c[nH]c3nc(NCC(C)(C)F)ncc23)ccc1=O] | 3 |
| 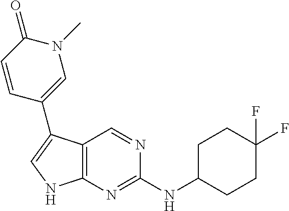 [SMILES: Cn1cc(-c2c[nH]c3nc(NC4CCC(F)(F)CC4)ncc23)ccc1=O] | 4 |
| 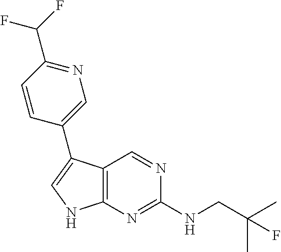 [SMILES: CC(C)(F)CNc1ncc2c(-c3ccc(C(F)F)nc3)c[nH]c2n1] | 5 |
| 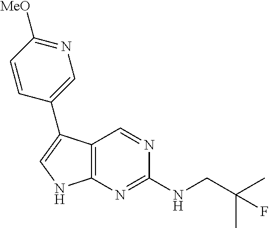 [SMILES: COc1ccc(-c2c[nH]c3nc(NCC(C)(C)F)ncc23)cn1] | 6 |
| 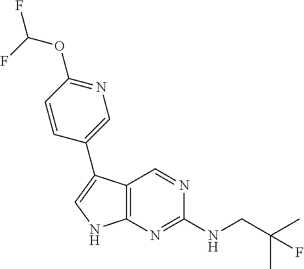 [SMILES: CC(C)(F)CNc1ncc2c(-c3ccc(OC(F)F)nc3)c[nH]c2n1] | 7 |
| 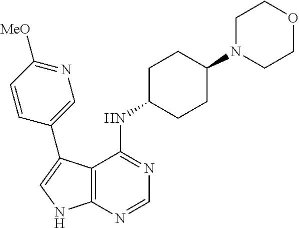 [SMILES: COc1ccc(-c2c[nH]c3ncnc(N[C@H]4CC[C@H](N5CCOCC5)CC4)c23)cn1] | 8 |
| 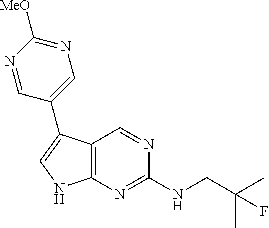 [SMILES: COc1ncc(-c2c[nH]c3nc(NCC(C)(C)F)ncc23)cn1] | 9 |
| 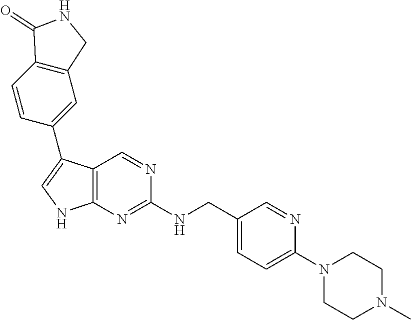 [SMILES: CN1CCN(c2ccc(CNc3ncc4c(-c5ccc6c(c5)CNC6=O)c[nH]c4n3)cn2)CC1] | 10 |
| 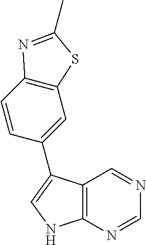 [SMILES: Cc1nc2ccc(-c3c[nH]c4ncncc34)cc2s1] | 11 |
| 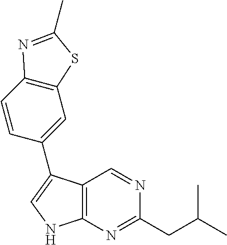 [SMILES: Cc1nc2ccc(-c3c[nH]c4nc(CC(C)C)ncc34)cc2s1] | 12 |
| 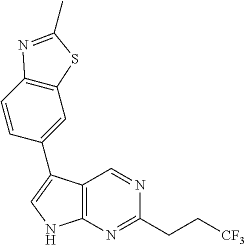 [SMILES: Cc1nc2ccc(-c3c[nH]c4nc(CCC(F)(F)F)ncc34)cc2s1] | 13 |
| 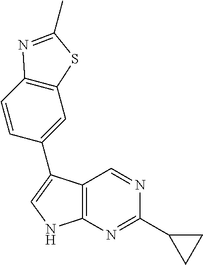 [SMILES: Cc1nc2ccc(-c3c[nH]c4nc(C5CC5)ncc34)cc2s1] | 14 |
| 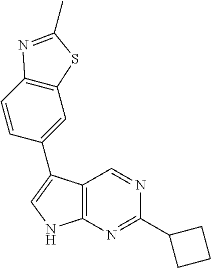 [SMILES: Cc1nc2ccc(-c3c[nH]c4nc(C5CCC5)ncc34)cc2s1] | 15 |
| 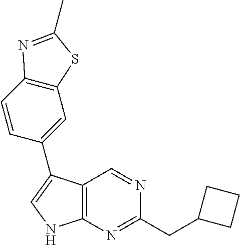 [SMILES: Cc1nc2ccc(-c3c[nH]c4nc(CC5CCC5)ncc34)cc2s1] | 16 |
| 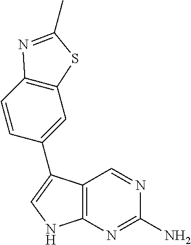 [SMILES: Cc1nc2ccc(-c3c[nH]c4nc(N)ncc34)cc2s1] | 17 |
| 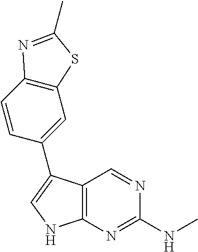 [SMILES: CNc1ncc2c(-c3ccc4nc(C)sc4c3)c[nH]c2n1] | 18 |
| 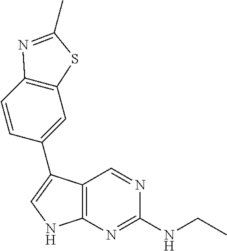 [SMILES: CCNc1ncc2c(-c3ccc4nc(C)sc4c3)c[nH]c2n1] | 19 |
| 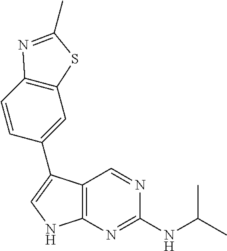 [SMILES: Cc1nc2ccc(-c3c[nH]c4nc(NC(C)C)ncc34)cc2s1] | 20 |
| 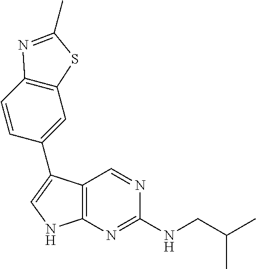 [SMILES: Cc1nc2ccc(-c3c[nH]c4nc(NCC(C)C)ncc34)cc2s1] | 21 |
| 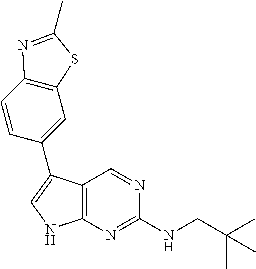 [SMILES: Cc1nc2ccc(-c3c[nH]c4nc(NCC(C)(C)C)ncc34)cc2s1] | 22 |
| 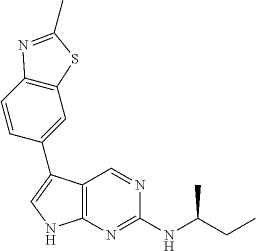 [SMILES: CC[C@H](C)Nc1ncc2c(-c3ccc4nc(C)sc4c3)c[nH]c2n1] | 23 |
| 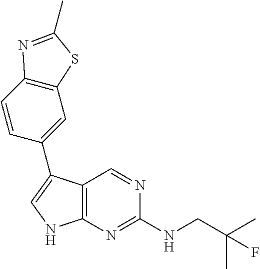 [SMILES: Cc1nc2ccc(-c3c[nH]c4nc(NCC(C)(C)F)ncc34)cc2s1] | 24 |
| 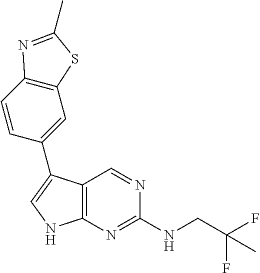 [SMILES: Cc1nc2ccc(-c3c[nH]c4nc(NCC(C)(F)F)ncc34)cc2s1] | 25 |
| 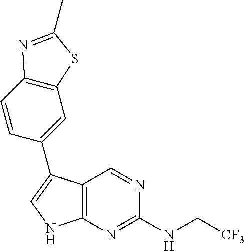 [SMILES: Cc1nc2ccc(-c3c[nH]c4nc(NCC(F)(F)F)ncc34)cc2s1] | 26 |
| 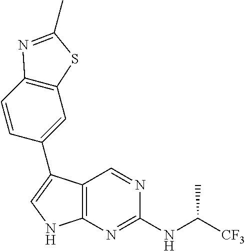 [SMILES: Cc1nc2ccc(-c3c[nH]c4nc(N[C@H](C)C(F)(F)F)ncc34)cc2s1] | 27 |
| 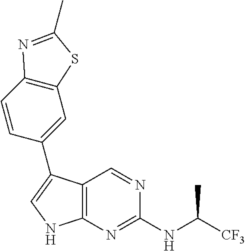 [SMILES: Cc1nc2ccc(-c3c[nH]c4nc(N[C@@H](C)C(F)(F)F)ncc34)cc2s1] | 28 |
| 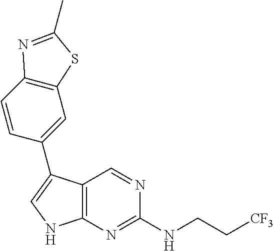 [SMILES: Cc1nc2ccc(-c3c[nH]c4nc(NCCC(F)(F)F)ncc34)cc2s1] | 29 |
| 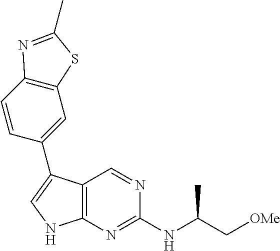 [SMILES: COC[C@H](C)Nc1ncc2c(-c3ccc4nc(C)sc4c3)c[nH]c2n1] | 30 |
| 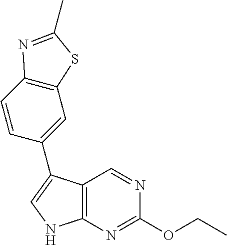 [SMILES: CCOc1ncc2c(-c3ccc4nc(C)sc4c3)c[nH]c2n1] | 31 |
| 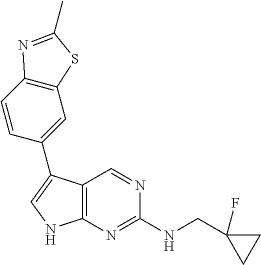 [SMILES: Cc1nc2ccc(-c3c[nH]c4nc(NCC5(F)CC5)ncc34)cc2s1] | 32 |
| 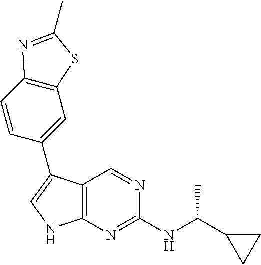 [SMILES: Cc1nc2ccc(-c3c[nH]c4nc(N[C@H](C)C5CC5)ncc34)cc2s1] | 33 |
| 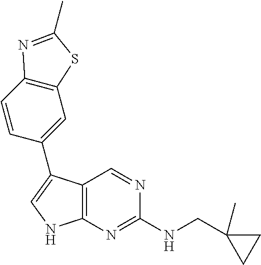 [SMILES: Cc1nc2ccc(-c3c[nH]c4nc(NCC5(C)CC5)ncc34)cc2s1] | 34 |
| 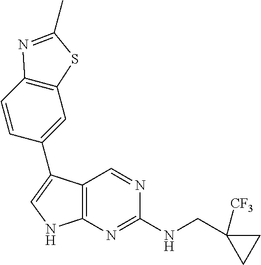 [SMILES: Cc1nc2ccc(-c3c[nH]c4nc(NCC5(C(F)(F)F)CC5)ncc34)cc2s1] | 35 |
| 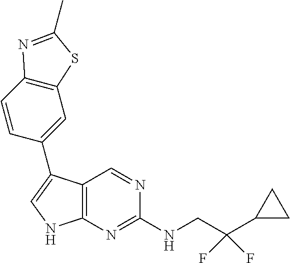 [SMILES: Cc1nc2ccc(-c3c[nH]c4nc(NCC(F)(F)C5CC5)ncc34)cc2s1] | 36 |
| 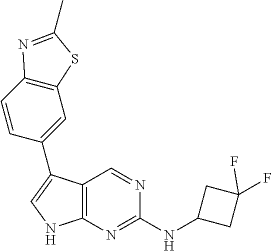 [SMILES: Cc1nc2ccc(-c3c[nH]c4nc(NC5CC(F)(F)C5)ncc34)cc2s1] | 37 |
| 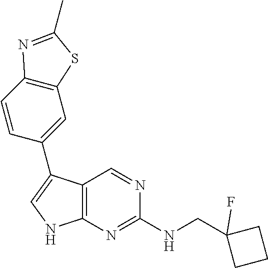 [SMILES: Cc1nc2ccc(-c3c[nH]c4nc(NCC5(F)CCC5)ncc34)cc2s1] | 38 |
| 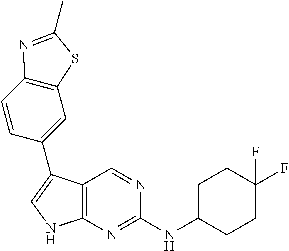 [SMILES: Cc1nc2ccc(-c3c[nH]c4nc(NC5CCC(F)(F)CC5)ncc34)cc2s1] | 39 |
| 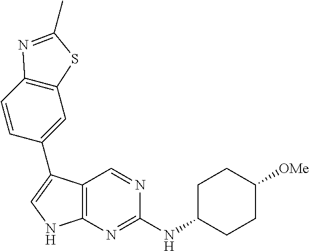 [SMILES: CO[C@H]1CC[C@@H](Nc2ncc3c(-c4ccc5nc(C)sc5c4)c[nH]c3n2)CC1] | 40 |
| 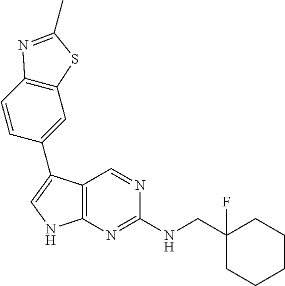 [SMILES: Cc1nc2ccc(-c3c[nH]c4nc(NCC5(F)CCCCC5)ncc34)cc2s1] | 41 |
| 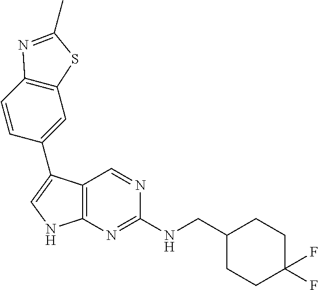 [SMILES: Cc1nc2ccc(-c3c[nH]c4nc(NCC5CCC(F)(F)CC5)ncc34)cc2s1] | 42 |
| 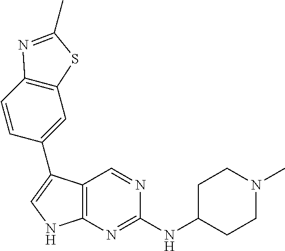 [SMILES: Cc1nc2ccc(-c3c[nH]c4nc(NC5CCN(C)CC5)ncc34)cc2s1] | 43 |
| 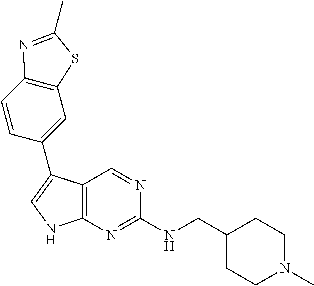 [SMILES: Cc1nc2ccc(-c3c[nH]c4nc(NCC5CCN(C)CC5)ncc34)cc2s1] | 44 |
| 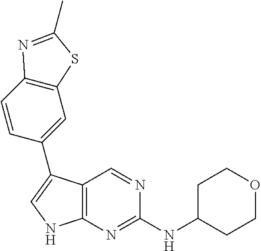 [SMILES: Cc1nc2ccc(-c3c[nH]c4nc(NC5CCOCC5)ncc34)cc2s1] | 45 |
| 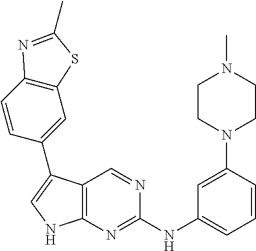 [SMILES: Cc1nc2ccc(-c3c[nH]c4nc(Nc5cccc(N6CCN(C)CC6)c5)ncc34)cc2s1] | 46 |
| 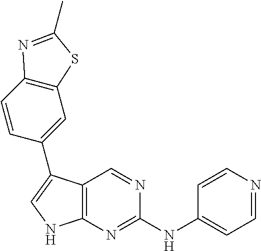 [SMILES: Cc1nc2ccc(-c3c[nH]c4nc(Nc5ccncc5)ncc34)cc2s1] | 47 |
| 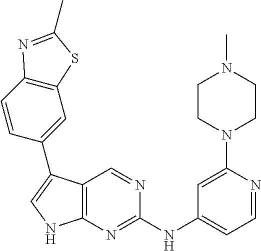 [SMILES: Cc1nc2ccc(-c3c[nH]c4nc(Nc5ccnc(N6CCN(C)CC6)c5)ncc34)cc2s1] | 48 |
| 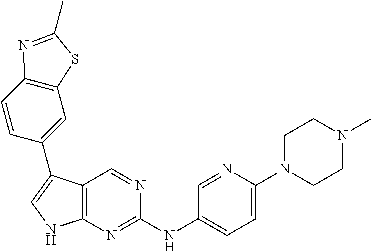 [SMILES: Cc1nc2ccc(-c3c[nH]c4nc(Nc5ccc(N6CCN(C)CC6)nc5)ncc34)cc2s1] | 49 |
| 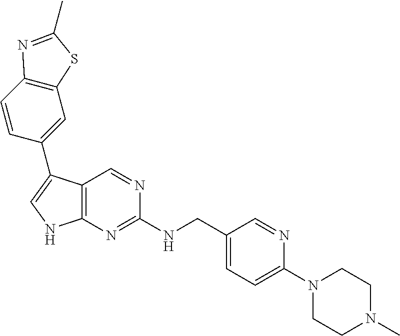 [SMILES: Cc1nc2ccc(-c3c[nH]c4nc(NCc5ccc(N6CCN(C)CC6)nc5)ncc34)cc2s1] | 50 |
| 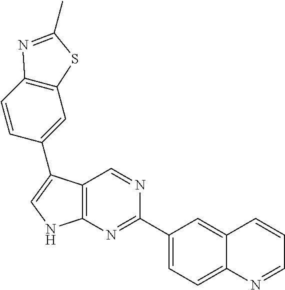 [SMILES: Cc1nc2ccc(-c3c[nH]c4nc(-c5ccc6ncccc6c5)ncc34)cc2s1] | 51 |
| 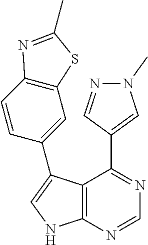 [SMILES: Cc1nc2ccc(-c3c[nH]c4ncnc(-c5cnn(C)c5)c34)cc2s1] | 52 |
| 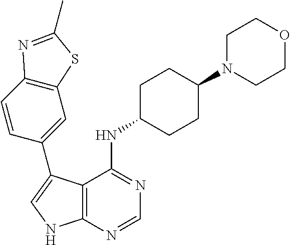 [SMILES: Cc1nc2ccc(-c3c[nH]c4ncnc(N[C@H]5CC[C@H](N6CCOCC6)CC5)c34)cc2s1] | 53 |
| 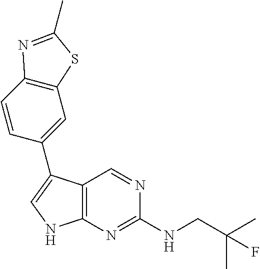 [SMILES: Cc1nc2ccc(-c3c[nH]c4nc(NCC(C)(C)F)ncc34)cc2s1] | 54 |
| 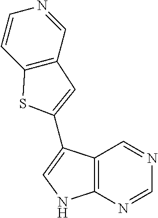 [SMILES: c1cc2sc(-c3c[nH]c4ncncc34)cc2cn1] | 55 |
| 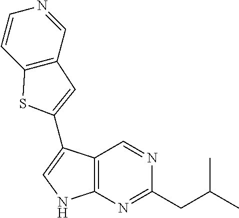 [SMILES: CC(C)Cc1ncc2c(-c3cc4cnccc4s3)c[nH]c2n1] | 56 |
| 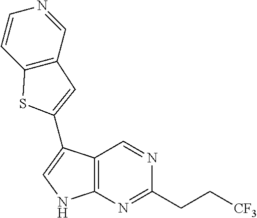 [SMILES: FC(F)(F)CCc1ncc2c(-c3cc4cnccc4s3)c[nH]c2n1] | 57 |
| 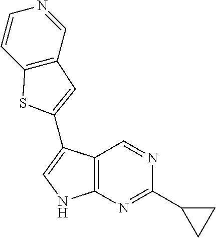 [SMILES: c1cc2sc(-c3c[nH]c4nc(C5CC5)ncc34)cc2cn1] | 58 |
| 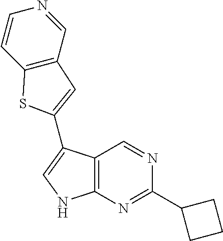 [SMILES: c1cc2sc(-c3c[nH]c4nc(C5CCC5)ncc34)cc2cn1] | 59 |
| 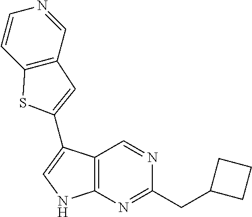 [SMILES: c1cc2sc(-c3c[nH]c4nc(CC5CCC5)ncc34)cc2cn1] | 60 |
| 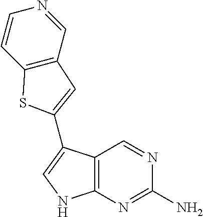 [SMILES: Nc1ncc2c(-c3cc4cnccc4s3)c[nH]c2n1] | 61 |
| 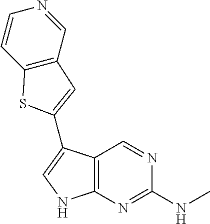 [SMILES: CNc1ncc2c(-c3cc4cnccc4s3)c[nH]c2n1] | 62 |
| 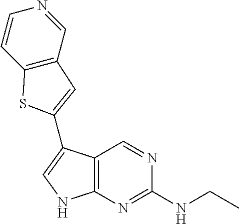 [SMILES: CCNc1ncc2c(-c3cc4cnccc4s3)c[nH]c2n1] | 63 |
| 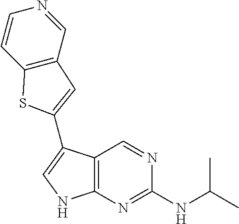 [SMILES: CC(C)Nc1ncc2c(-c3cc4cnccc4s3)c[nH]c2n1] | 64 |
| 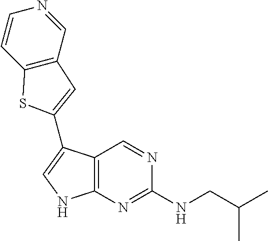 [SMILES: CC(C)CNc1ncc2c(-c3cc4cnccc4s3)c[nH]c2n1] | 65 |
| 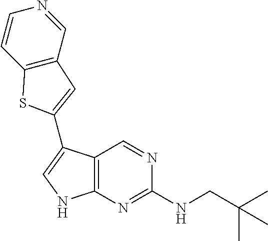 [SMILES: CC(C)(C)CNc1ncc2c(-c3cc4cnccc4s3)c[nH]c2n1] | 66 |
| 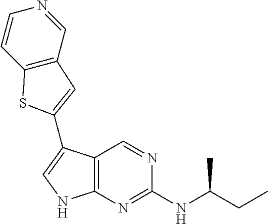 [SMILES: CC[C@H](C)Nc1ncc2c(-c3cc4cnccc4s3)c[nH]c2n1] | 67 |
| 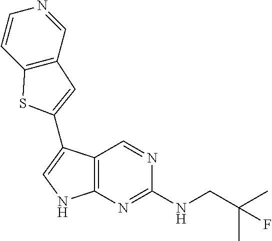 [SMILES: CC(C)(F)CNc1ncc2c(-c3cc4cnccc4s3)c[nH]c2n1] | 68 |
| 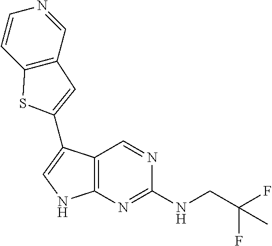 [SMILES: CC(F)(F)CNc1ncc2c(-c3cc4cnccc4s3)c[nH]c2n1] | 69 |
| 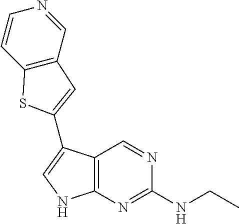 [SMILES: CCNc1ncc2c(-c3cc4cnccc4s3)c[nH]c2n1] | 70 |
| 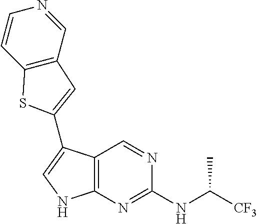 [SMILES: C[C@@H](Nc1ncc2c(-c3cc4cnccc4s3)c[nH]c2n1)C(F)(F)F] | 71 |
| 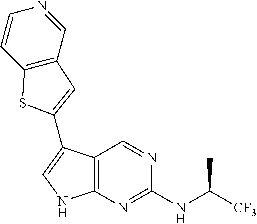 [SMILES: C[C@H](Nc1ncc2c(-c3cc4cnccc4s3)c[nH]c2n1)C(F)(F)F] | 72 |
| 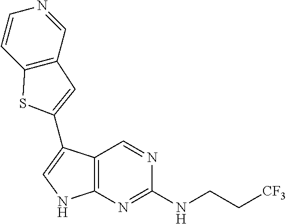 [SMILES: FC(F)(F)CCNc1ncc2c(-c3cc4cnccc4s3)c[nH]c2n1] | 73 |
| 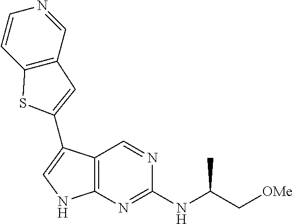 [SMILES: COC[C@H](C)Nc1ncc2c(-c3cc4cnccc4s3)c[nH]c2n1] | 74 |
| 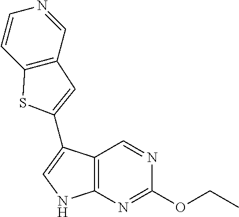 [SMILES: CCOc1ncc2c(-c3cc4cnccc4s3)c[nH]c2n1] | 75 |
| 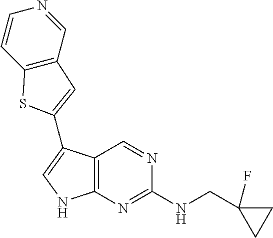 [SMILES: FC1(CNc2ncc3c(-c4cc5cnccc5s4)c[nH]c3n2)CC1] | 76 |
| 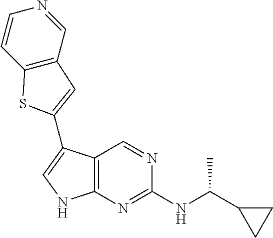 [SMILES: C[C@@H](Nc1ncc2c(-c3cc4cnccc4s3)c[nH]c2n1)C1CC1] | 77 |
| 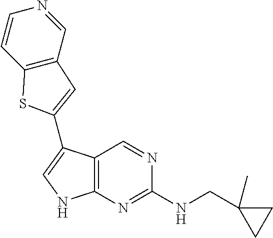 [SMILES: CC1(CNc2ncc3c(-c4cc5cnccc5s4)c[nH]c3n2)CC1] | 78 |
| 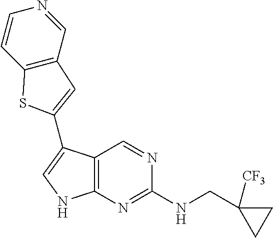 [SMILES: FC(F)(F)C1(CNc2ncc3c(-c4cc5cnccc5s4)c[nH]c3n2)CC1] | 79 |
| 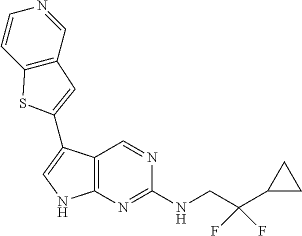 [SMILES: FC(F)(CNc1ncc2c(-c3cc4cnccc4s3)c[nH]c2n1)C1CC1] | 80 |
| 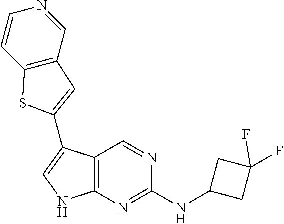 [SMILES: FC1(F)CC(Nc2ncc3c(-c4cc5cnccc5s4)c[nH]c3n2)C1] | 81 |
| 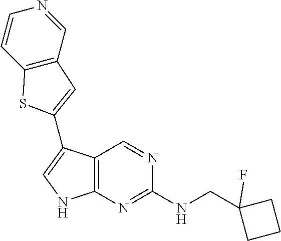 [SMILES: FC1(CNc2ncc3c(-c4cc5cnccc5s4)c[nH]c3n2)CCC1] | 82 |
| 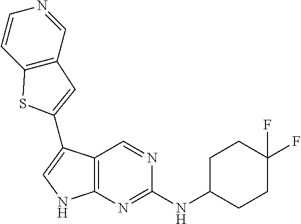 [SMILES: FC1(F)CCC(Nc2ncc3c(-c4cc5cnccc5s4)c[nH]c3n2)CC1] | 83 |
| 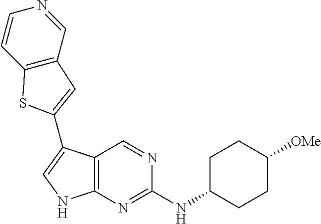 [SMILES: CO[C@H]1CC[C@@H](Nc2ncc3c(-c4cc5cnccc5s4)c[nH]c3n2)CC1] | 84 |
| 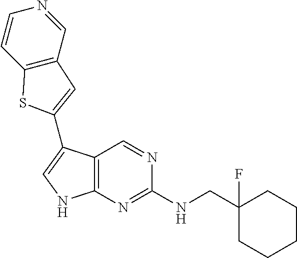 [SMILES: FC1(CNc2ncc3c(-c4cc5cnccc5s4)c[nH]c3n2)CCCCC1] | 85 |
| 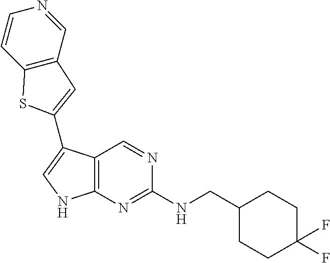 [SMILES: FC1(F)CCC(CNc2ncc3c(-c4cc5cnccc5s4)c[nH]c3n2)CC1] | 86 |
| 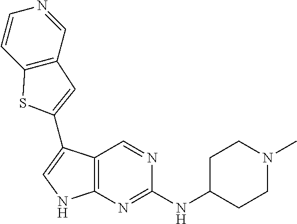 [SMILES: CN1CCC(Nc2ncc3c(-c4cc5cnccc5s4)c[nH]c3n2)CC1] | 87 |
| 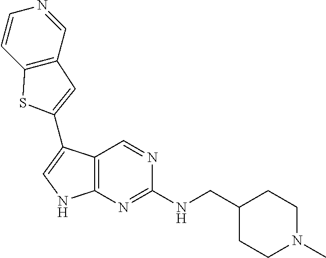 [SMILES: CN1CCC(CNc2ncc3c(-c4cc5cnccc5s4)c[nH]c3n2)CC1] | 88 |
| 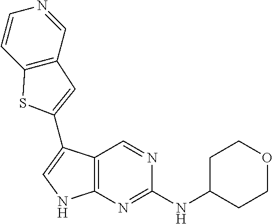 [SMILES: c1cc2sc(-c3c[nH]c4nc(NC5CCOCC5)ncc34)cc2cn1] | 89 |
| 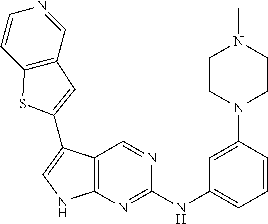 [SMILES: CN1CCN(c2cccc(Nc3ncc4c(-c5cc6cnccc6s5)c[nH]c4n3)c2)CC1] | 90 |
| 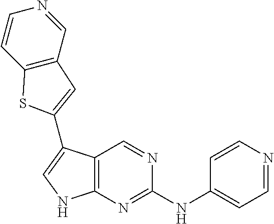 [SMILES: c1cc(Nc2ncc3c(-c4cc5cnccc5s4)c[nH]c3n2)ccn1] | 91 |
| 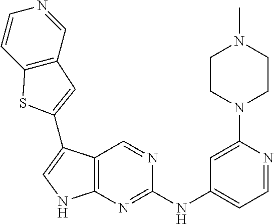 [SMILES: CN1CCN(c2cc(Nc3ncc4c(-c5cc6cnccc6s5)c[nH]c4n3)ccn2)CC1] | 92 |
| 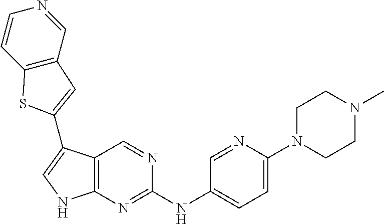 [SMILES: CN1CCN(c2ccc(Nc3ncc4c(-c5cc6cnccc6s5)c[nH]c4n3)cn2)CC1] | 93 |
| 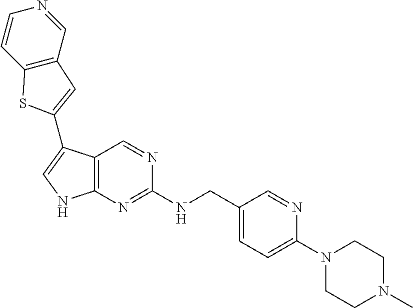 [SMILES: CN1CCN(c2ccc(CNc3ncc4c(-c5cc6cnccc6s5)c[nH]c4n3)cn2)CC1] | 94 |
| 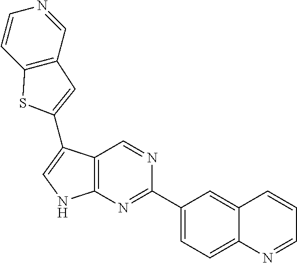 [SMILES: c1cnc2ccc(-c3ncc4c(-c5cc6cnccc6s5)c[nH]c4n3)cc2c1] | 95 |
| 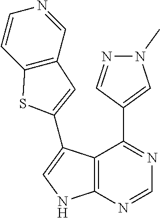 [SMILES: Cn1cc(-c2ncnc3[nH]cc(-c4cc5cnccc5s4)c23)cn1] | 96 |
| 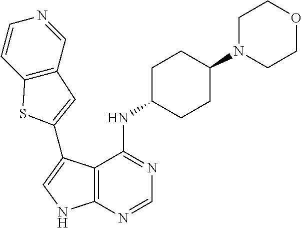 [SMILES: c1cc2sc(-c3c[nH]c4ncnc(N[C@H]5CC[C@H](N6CCOCC6)CC5)c34)cc2cn1] | 97 |
| 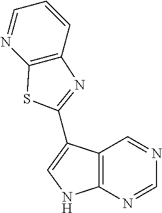 [SMILES: c1cnc2sc(-c3c[nH]c4ncncc34)nc2c1] | 98 |
| 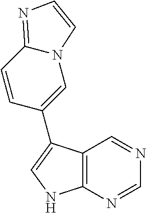 [SMILES: c1ncc2c(-c3ccc4nccn4c3)c[nH]c2n1] | 99 |
| 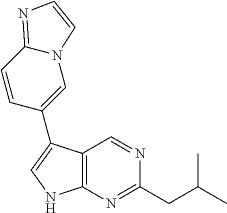 [SMILES: CC(C)Cc1ncc2c(-c3ccc4nccn4c3)c[nH]c2n1] | 100 |
| 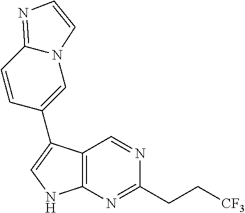 [SMILES: FC(F)(F)CCc1ncc2c(-c3ccc4nccn4c3)c[nH]c2n1] | 101 |
| 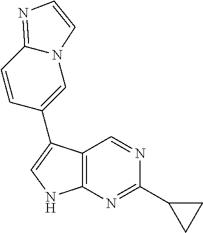 [SMILES: c1cn2cc(-c3c[nH]c4nc(C5CC5)ncc34)ccc2n1] | 102 |
| 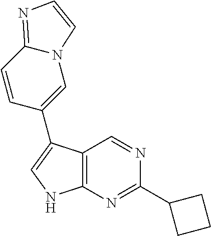 [SMILES: c1cn2cc(-c3c[nH]c4nc(C5CCC5)ncc34)ccc2n1] | 103 |
| 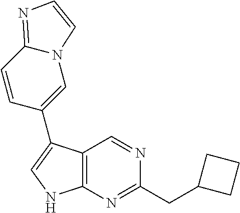 [SMILES: c1cn2cc(-c3c[nH]c4nc(CC5CCC5)ncc34)ccc2n1] | 104 |
| 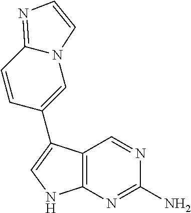 [SMILES: Nc1ncc2c(-c3ccc4nccn4c3)c[nH]c2n1] | 105 |
| 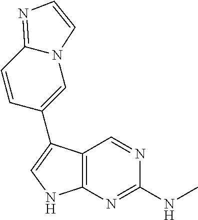 [SMILES: CNc1ncc2c(-c3ccc4nccn4c3)c[nH]c2n1] | 106 |
| 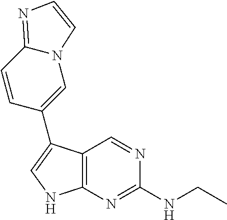 [SMILES: CCNc1ncc2c(-c3ccc4nccn4c3)c[nH]c2n1] | 107 |
| 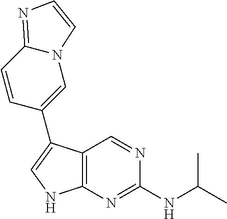 [SMILES: CC(C)Nc1ncc2c(-c3ccc4nccn4c3)c[nH]c2n1] | 108 |
| 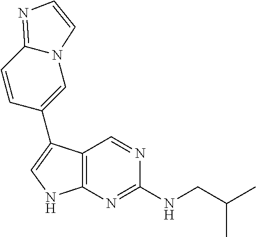 [SMILES: CC(C)CNc1ncc2c(-c3ccc4nccn4c3)c[nH]c2n1] | 109 |
| 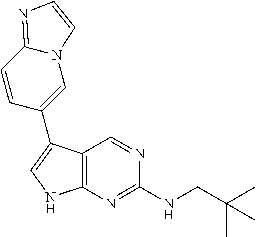 [SMILES: CC(C)(C)CNc1ncc2c(-c3ccc4nccn4c3)c[nH]c2n1] | 110 |
| 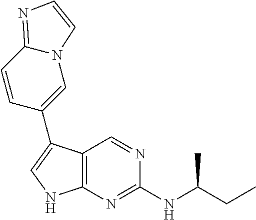 [SMILES: CC[C@H](C)Nc1ncc2c(-c3ccc4nccn4c3)c[nH]c2n1] | 111 |
| 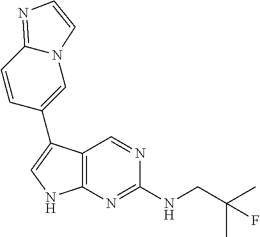 [SMILES: CC(C)(F)CNc1ncc2c(-c3ccc4nccn4c3)c[nH]c2n1] | 112 |
| 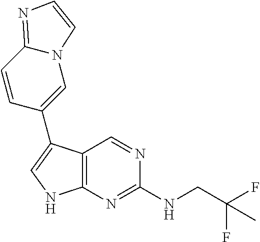 [SMILES: CC(F)(F)CNc1ncc2c(-c3ccc4nccn4c3)c[nH]c2n1] | 113 |
| 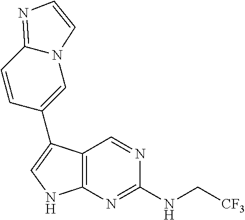 [SMILES: FC(F)(F)CNc1ncc2c(-c3ccc4nccn4c3)c[nH]c2n1] | 114 |
| 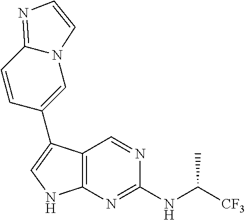 [SMILES: C[C@@H](Nc1ncc2c(-c3ccc4nccn4c3)c[nH]c2n1)C(F)(F)F] | 115 |
| 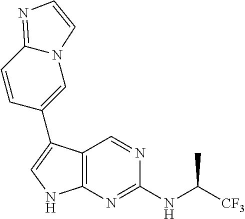 [SMILES: C[C@H](Nc1ncc2c(-c3ccc4nccn4c3)c[nH]c2n1)C(F)(F)F] | 116 |
| 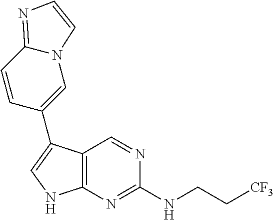 [SMILES: FC(F)(F)CCNc1ncc2c(-c3ccc4nccn4c3)c[nH]c2n1] | 117 |
| 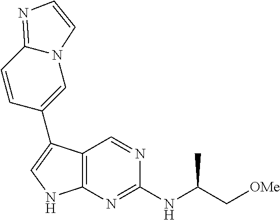 [SMILES: COC[C@H](C)Nc1ncc2c(-c3ccc4nccn4c3)c[nH]c2n1] | 118 |
| 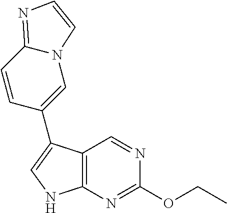 [SMILES: CCOc1ncc2c(-c3ccc4nccn4c3)c[nH]c2n1] | 119 |
| 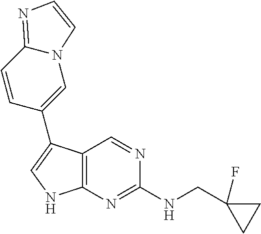 [SMILES: FC1(CNc2ncc3c(-c4ccc5nccn5c4)c[nH]c3n2)CC1] | 120 |
| 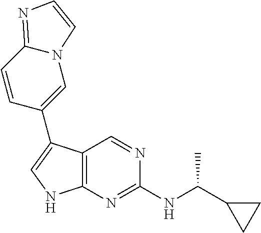 [SMILES: C[C@@H](Nc1ncc2c(-c3ccc4nccn4c3)c[nH]c2n1)C1CC1] | 121 |
| 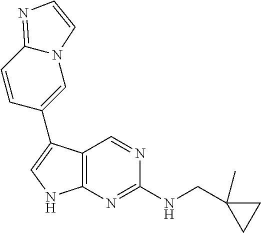 [SMILES: CC1(CNc2ncc3c(-c4ccc5nccn5c4)c[nH]c3n2)CC1] | 122 |
| 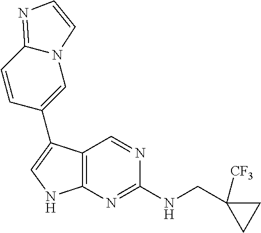 [SMILES: FC(F)(F)C1(CNc2ncc3c(-c4ccc5nccn5c4)c[nH]c3n2)CC1] | 123 |
| 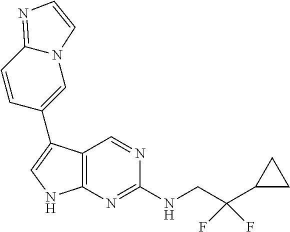 [SMILES: FC(F)(CNc1ncc2c(-c3ccc4nccn4c3)c[nH]c2n1)C1CC1] | 124 |
| 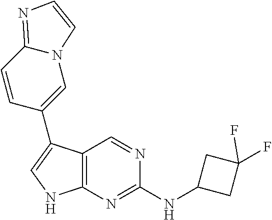 [SMILES: FC1(F)CC(Nc2ncc3c(-c4ccc5nccn5c4)c[nH]c3n2)C1] | 125 |
| 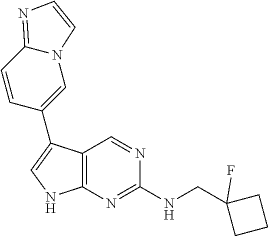 [SMILES: FC1(CNc2ncc3c(-c4ccc5nccn5c4)c[nH]c3n2)CCC1] | 126 |
| 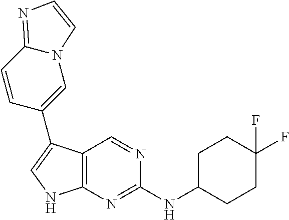 [SMILES: FC1(F)CCC(Nc2ncc3c(-c4ccc5nccn5c4)c[nH]c3n2)CC1] | 127 |
| 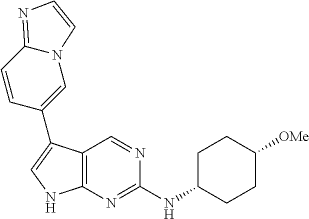 [SMILES: CO[C@H]1CC[C@@H](Nc2ncc3c(-c4ccc5nccn5c4)c[nH]c3n2)CC1] | 128 |
| 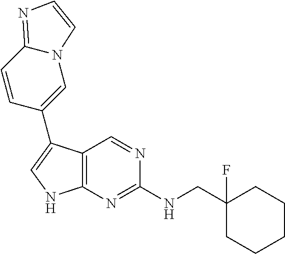 [SMILES: FC1(CNc2ncc3c(-c4ccc5nccn5c4)c[nH]c3n2)CCCCC1] | 129 |
| 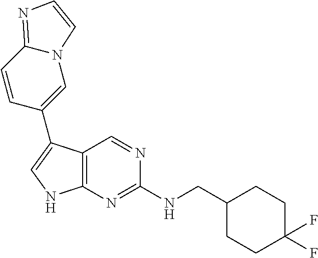 [SMILES: FC1(F)CCC(CNc2ncc3c(-c4ccc5nccn5c4)c[nH]c3n2)CC1] | 130 |
| 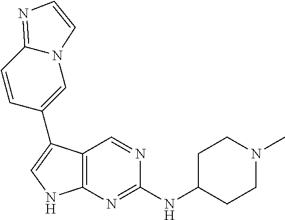 [SMILES: CN1CCC(Nc2ncc3c(-c4ccc5nccn5c4)c[nH]c3n2)CC1] | 131 |
| 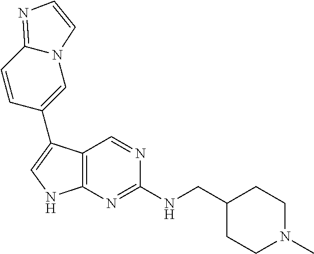 [SMILES: CN1CCC(CNc2ncc3c(-c4ccc5nccn5c4)c[nH]c3n2)CC1] | 132 |
| 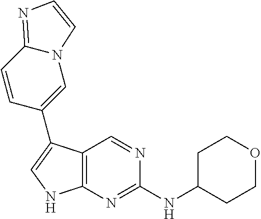 [SMILES: c1cn2cc(-c3c[nH]c4nc(NC5CCOCC5)ncc34)ccc2n1] | 133 |
| 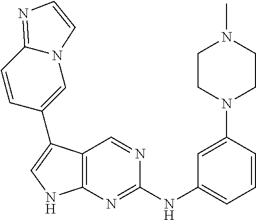 [SMILES: CN1CCN(c2cccc(Nc3ncc4c(-c5ccc6nccn6c5)c[nH]c4n3)c2)CC1] | 134 |
| 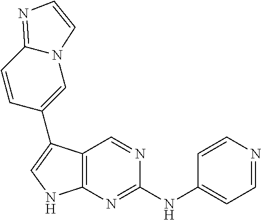 [SMILES: c1cc(Nc2ncc3c(-c4ccc5nccn5c4)c[nH]c3n2)ccn1] | 135 |
| 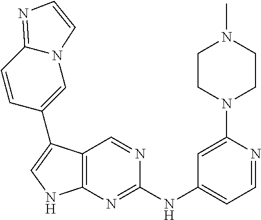 [SMILES: CN1CCN(c2cc(Nc3ncc4c(-c5ccc6nccn6c5)c[nH]c4n3)ccn2)CC1] | 136 |
| 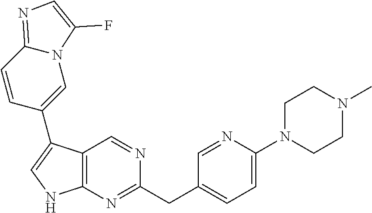 [SMILES: CN1CCN(c2ccc(Cc3ncc4c(-c5ccc6ncc(F)n6c5)c[nH]c4n3)cn2)CC1] | 137 |
| 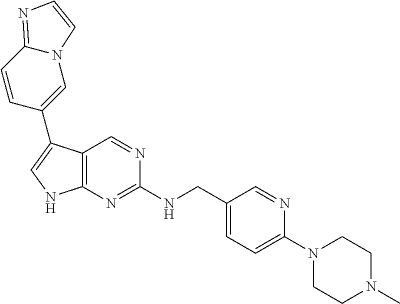 [SMILES: CN1CCN(c2ccc(CNc3ncc4c(-c5ccc6nccn6c5)c[nH]c4n3)cn2)CC1] | 138 |
| 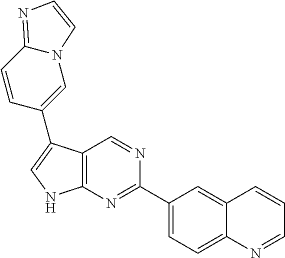 [SMILES: c1cnc2ccc(-c3ncc4c(-c5ccc6nccn6c5)c[nH]c4n3)cc2c1] | 139 |
| 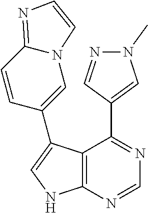 [SMILES: Cn1cc(-c2ncnc3[nH]cc(-c4ccc5nccn5c4)c23)cn1] | 140 |
| 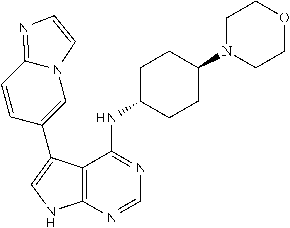 [SMILES: c1nc(N[C@H]2CC[C@H](N3CCOCC3)CC2)c2c(-c3ccc4nccn4c3)c[nH]c2n1] | 141 |
| 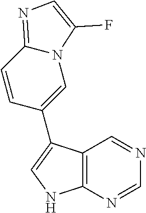 [SMILES: Fc1cnc2ccc(-c3c[nH]c4ncncc34)cn12] | 142 |
| 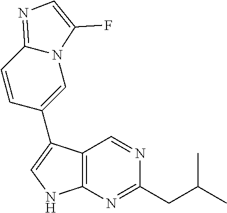 [SMILES: CC(C)Cc1ncc2c(-c3ccc4ncc(F)n4c3)c[nH]c2n1] | 143 |
| 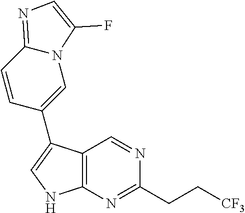 [SMILES: Fc1cnc2ccc(-c3c[nH]c4nc(CCC(F)(F)F)ncc34)cn12] | 144 |
| 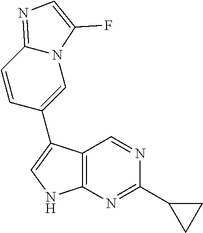 [SMILES: Fc1cnc2ccc(-c3c[nH]c4nc(C5CC5)ncc34)cn12] | 145 |
| 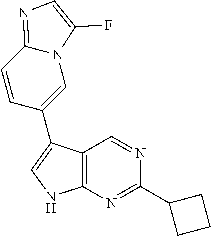 [SMILES: Fc1cnc2ccc(-c3c[nH]c4nc(C5CCC5)ncc34)cn12] | 146 |
| 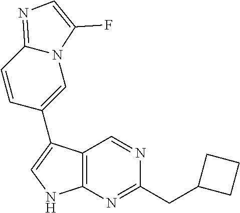 [SMILES: Fc1cnc2ccc(-c3c[nH]c4nc(CC5CCC5)ncc34)cn12] | 147 |
| 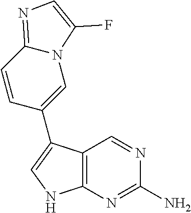 [SMILES: Nc1ncc2c(-c3ccc4ncc(F)n4c3)c[nH]c2n1] | 148 |
| 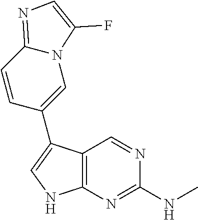 [SMILES: CNc1ncc2c(-c3ccc4ncc(F)n4c3)c[nH]c2n1] | 149 |
| 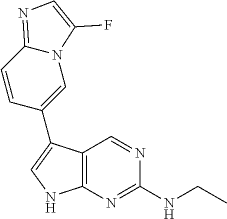 [SMILES: CCNc1ncc2c(-c3ccc4ncc(F)n4c3)c[nH]c2n1] | 150 |
| 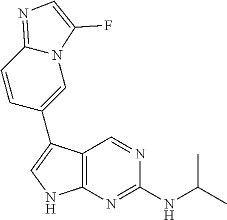 [SMILES: CC(C)Nc1ncc2c(-c3ccc4ncc(F)n4c3)c[nH]c2n1] | 151 |
| 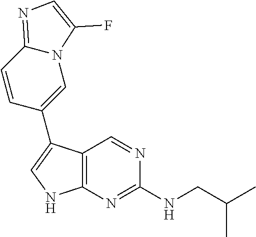 [SMILES: CC(C)CNc1ncc2c(-c3ccc4ncc(F)n4c3)c[nH]c2n1] | 152 |
| 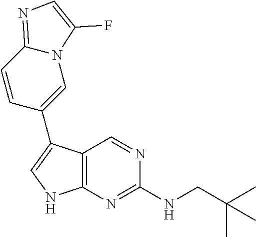 [SMILES: CC(C)(C)CNc1ncc2c(-c3ccc4ncc(F)n4c3)c[nH]c2n1] | 153 |
| 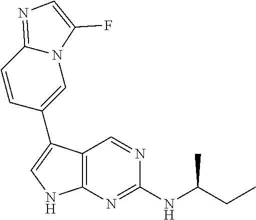 [SMILES: CC[C@H](C)Nc1ncc2c(-c3ccc4ncc(F)n4c3)c[nH]c2n1] | 154 |
| 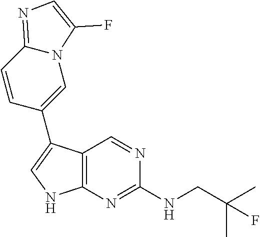 [SMILES: CC(C)(F)CNc1ncc2c(-c3ccc4ncc(F)n4c3)c[nH]c2n1] | 155 |
| 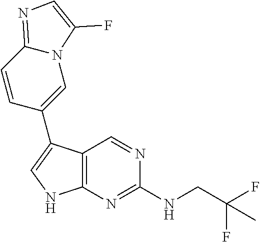 [SMILES: CC(F)(F)CNc1ncc2c(-c3ccc4ncc(F)n4c3)c[nH]c2n1] | 156 |
| 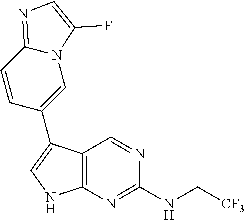 [SMILES: Fc1cnc2ccc(-c3c[nH]c4nc(NCC(F)(F)F)ncc34)cn12] | 157 |
| 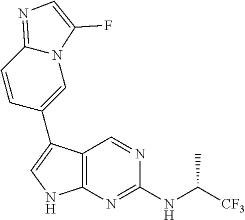 [SMILES: C[C@@H](Nc1ncc2c(-c3ccc4ncc(F)n4c3)c[nH]c2n1)C(F)(F)F] | 158 |
| 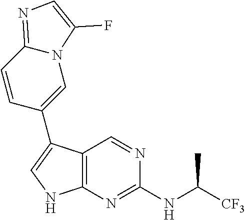 [SMILES: C[C@H](Nc1ncc2c(-c3ccc4ncc(F)n4c3)c[nH]c2n1)C(F)(F)F] | 159 |
| 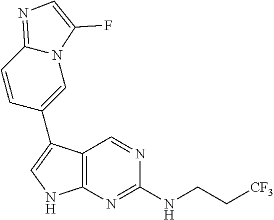 [SMILES: Fc1cnc2ccc(-c3c[nH]c4nc(NCCC(F)(F)F)ncc34)cn12] | 160 |
| 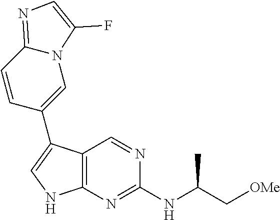 [SMILES: COC[C@H](C)Nc1ncc2c(-c3ccc4ncc(F)n4c3)c[nH]c2n1] | 161 |
| 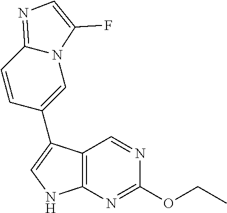 [SMILES: CCOc1ncc2c(-c3ccc4ncc(F)n4c3)c[nH]c2n1] | 162 |
| 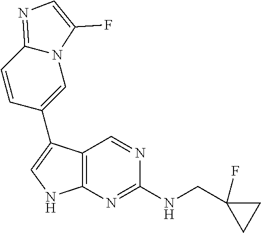 [SMILES: Fc1cnc2ccc(-c3c[nH]c4nc(NCC5(F)CC5)ncc34)cn12] | 163 |
| 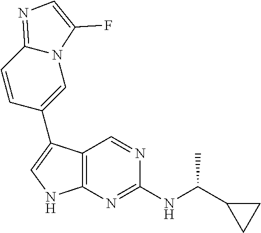 [SMILES: C[C@@H](Nc1ncc2c(-c3ccc4ncc(F)n4c3)c[nH]c2n1)C1CC1] | 164 |
| 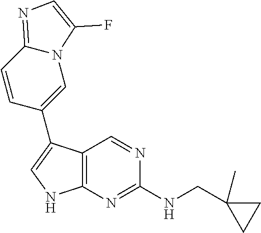 [SMILES: CC1(CNc2ncc3c(-c4ccc5ncc(F)n5c4)c[nH]c3n2)CC1] | 165 |
| 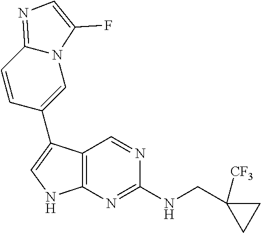 [SMILES: Fc1cnc2ccc(-c3c[nH]c4nc(NCC5(C(F)(F)F)CC5)ncc34)cn12] | 166 |
| 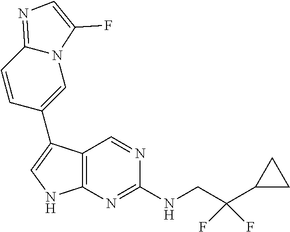 [SMILES: Fc1cnc2ccc(-c3c[nH]c4nc(NCC(F)(F)C5CC5)ncc34)cn12] | 167 |
| 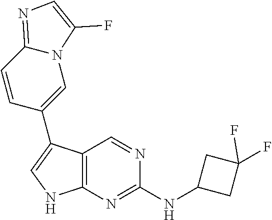 [SMILES: Fc1cnc2ccc(-c3c[nH]c4nc(NC5CC(F)(F)C5)ncc34)cn12] | 168 |
| 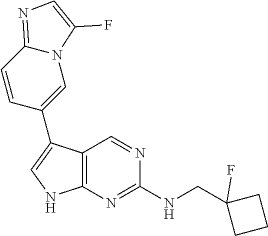 [SMILES: Fc1cnc2ccc(-c3c[nH]c4nc(NCC5(F)CCC5)ncc34)cn12] | 169 |
| 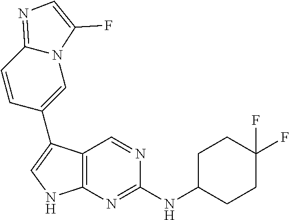 [SMILES: Fc1cnc2ccc(-c3c[nH]c4nc(NC5CCC(F)(F)CC5)ncc34)cn12] | 170 |
| 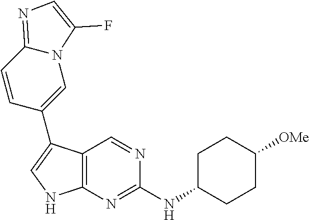 [SMILES: CO[C@H]1CC[C@@H](Nc2ncc3c(-c4ccc5ncc(F)n5c4)c[nH]c3n2)CC1] | 171 |
| 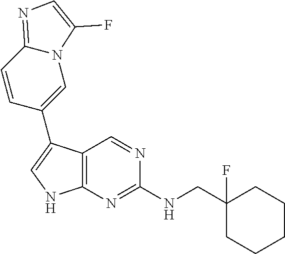 [SMILES: Fc1cnc2ccc(-c3c[nH]c4nc(NCC5(F)CCCCC5)ncc34)cn12] | 172 |
| 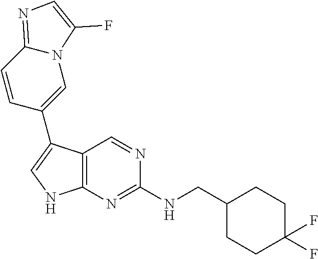 [SMILES: Fc1cnc2ccc(-c3c[nH]c4nc(NCC5CCC(F)(F)CC5)ncc34)cn12] | 173 |
| 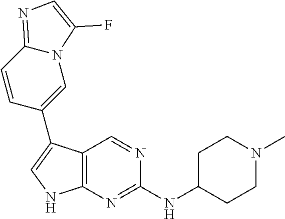 [SMILES: CN1CCC(Nc2ncc3c(-c4ccc5ncc(F)n5c4)c[nH]c3n2)CC1] | 174 |
| 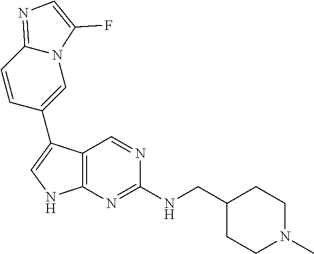 [SMILES: CN1CCC(CNc2ncc3c(-c4ccc5ncc(F)n5c4)c[nH]c3n2)CC1] | 175 |
| 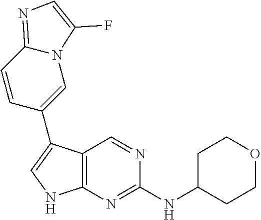 [SMILES: Fc1cnc2ccc(-c3c[nH]c4nc(NC5CCOCC5)ncc34)cn12] | 176 |
| 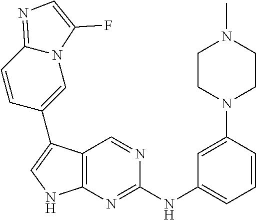 [SMILES: CN1CCN(c2cccc(Nc3ncc4c(-c5ccc6ncc(F)n6c5)c[nH]c4n3)c2)CC1] | 177 |
| 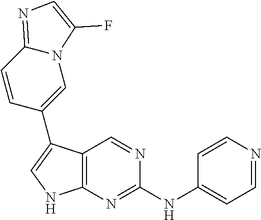 [SMILES: Fc1cnc2ccc(-c3c[nH]c4nc(Nc5ccncc5)ncc34)cn12] | 178 |
| 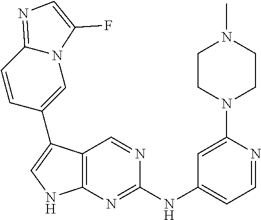 [SMILES: CN1CCN(c2cc(Nc3ncc4c(-c5ccc6ncc(F)n6c5)c[nH]c4n3)ccn2)CC1] | 179 |
| 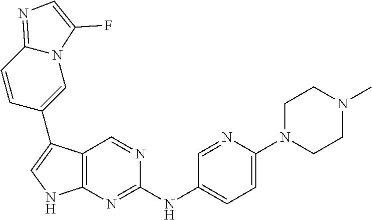 [SMILES: CN1CCN(c2ccc(Nc3ncc4c(-c5ccc6ncc(F)n6c5)c[nH]c4n3)cn2)CC1] | 180 |
| 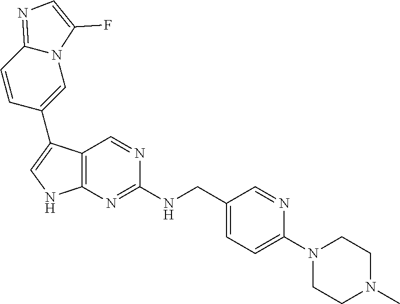 [SMILES: CN1CCN(c2ccc(CNc3ncc4c(-c5ccc6ncc(F)n6c5)c[nH]c4n3)cn2)CC1] | 181 |
| 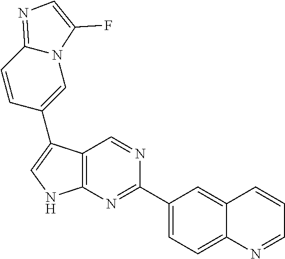 [SMILES: Fc1cnc2ccc(-c3c[nH]c4nc(-c5ccc6ncccc6c5)ncc34)cn12] | 182 |
| 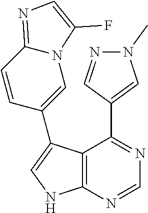 [SMILES: Cn1cc(-c2ncnc3[nH]cc(-c4ccc5ncc(F)n5c4)c23)cn1] | 183 |
| 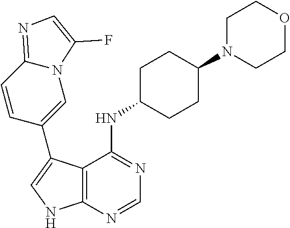 [SMILES: Fc1cnc2ccc(-c3c[nH]c4ncnc(N[C@H]5CC[C@H](N6CCOCC6)CC5)c34)cn12] | 184 |
| 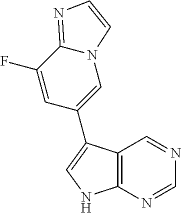 [SMILES: Fc1cc(-c2c[nH]c3ncncc23)cn2ccnc12] | 185 |
| 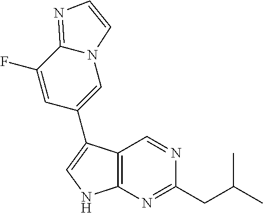 [SMILES: CC(C)Cc1ncc2c(-c3cc(F)c4nccn4c3)c[nH]c2n1] | 186 |
| 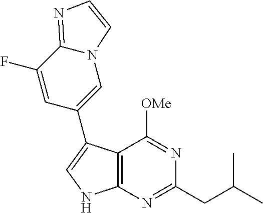 [SMILES: COc1nc(CC(C)C)nc2[nH]cc(-c3cc(F)c4nccn4c3)c12] | 187 |
| 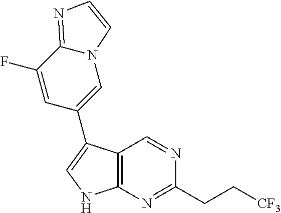 [SMILES: Fc1cc(-c2c[nH]c3nc(CCC(F)(F)F)ncc23)cn2ccnc12] | 188 |
| 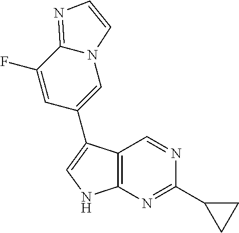 [SMILES: Fc1cc(-c2c[nH]c3nc(C4CC4)ncc23)cn2ccnc12] | 189 |
| 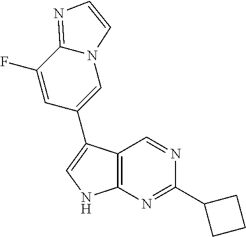 [SMILES: Fc1cc(-c2c[nH]c3nc(C4CCC4)ncc23)cn2ccnc12] | 190 |
| 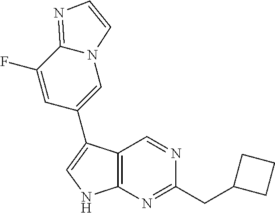 [SMILES: Fc1cc(-c2c[nH]c3nc(CC4CCC4)ncc23)cn2ccnc12] | 191 |
| 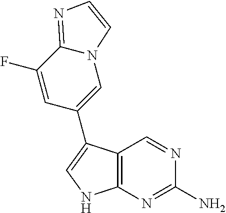 [SMILES: Nc1ncc2c(-c3cc(F)c4nccn4c3)c[nH]c2n1] | 192 |
| 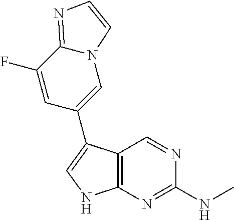 [SMILES: CNc1ncc2c(-c3cc(F)c4nccn4c3)c[nH]c2n1] | 193 |
| 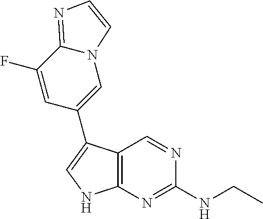 [SMILES: CCNc1ncc2c(-c3cc(F)c4nccn4c3)c[nH]c2n1] | 194 |
| 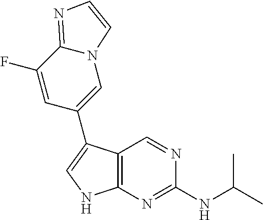 [SMILES: CC(C)Nc1ncc2c(-c3cc(F)c4nccn4c3)c[nH]c2n1] | 195 |
| 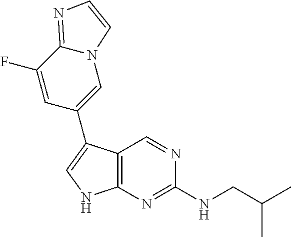 [SMILES: CC(C)CNc1ncc2c(-c3cc(F)c4nccn4c3)c[nH]c2n1] | 196 |
| 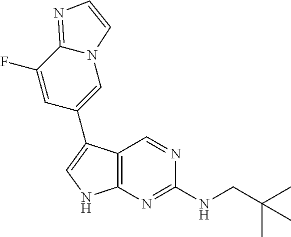 [SMILES: CC(C)(C)CNc1ncc2c(-c3cc(F)c4nccn4c3)c[nH]c2n1] | 197 |
| 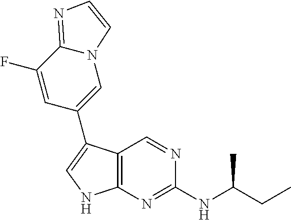 [SMILES: CC[C@H](C)Nc1ncc2c(-c3cc(F)c4nccn4c3)c[nH]c2n1] | 198 |
| 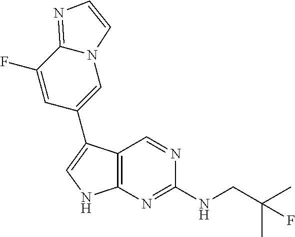 [SMILES: CC(C)(F)CNc1ncc2c(-c3cc(F)c4nccn4c3)c[nH]c2n1] | 199 |
| 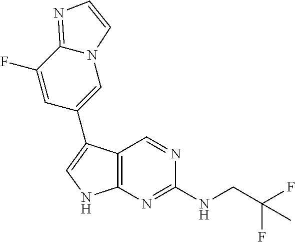 [SMILES: CC(F)(F)CNc1ncc2c(-c3cc(F)c4nccn4c3)c[nH]c2n1] | 200 |
| 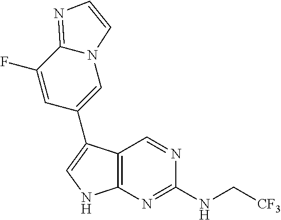 [SMILES: Fc1cc(-c2c[nH]c3nc(NCC(F)(F)F)ncc23)cn2ccnc12] | 201 |
| 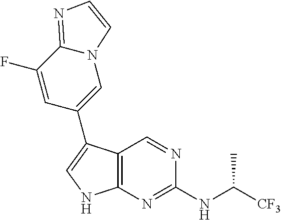 [SMILES: C[C@@H](Nc1ncc2c(-c3cc(F)c4nccn4c3)c[nH]c2n1)C(F)(F)F] | 202 |
| 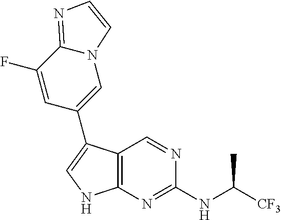 [SMILES: C[C@H](Nc1ncc2c(-c3cc(F)c4nccn4c3)c[nH]c2n1)C(F)(F)F] | 203 |
| 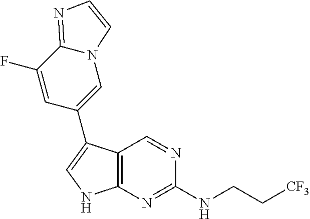 [SMILES: Fc1cc(-c2c[nH]c3nc(NCCC(F)(F)F)ncc23)cn2ccnc12] | 204 |
| 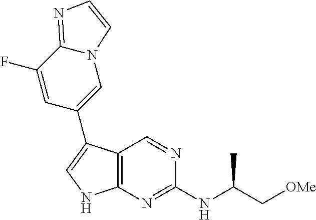 [SMILES: COC[C@H](C)Nc1ncc2c(-c3cc(F)c4nccn4c3)c[nH]c2n1] | 205 |
| 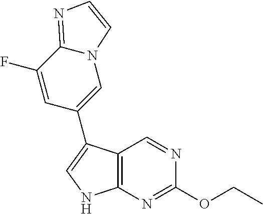 [SMILES: CCOc1ncc2c(-c3cc(F)c4nccn4c3)c[nH]c2n1] | 206 |
| 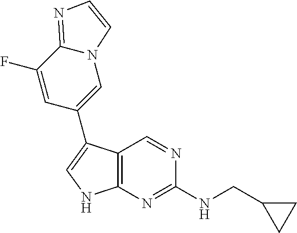 [SMILES: Fc1cc(-c2c[nH]c3nc(NCC4CC4)ncc23)cn2ccnc12] | 207 |
| 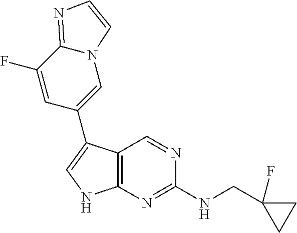 [SMILES: Fc1cc(-c2c[nH]c3nc(NCC4(F)CC4)ncc23)cn2ccnc12] | 208 |
| 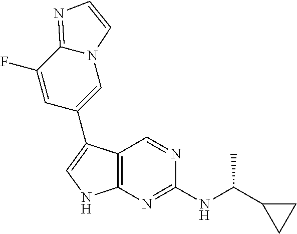 [SMILES: C[C@@H](Nc1ncc2c(-c3cc(F)c4nccn4c3)c[nH]c2n1)C1CC1] | 209 |
| 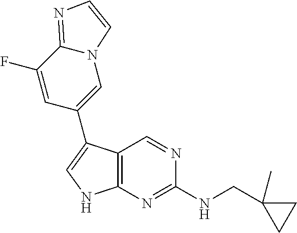 [SMILES: CC1(CNc2ncc3c(-c4cc(F)c5nccn5c4)c[nH]c3n2)CC1] | 210 |
| 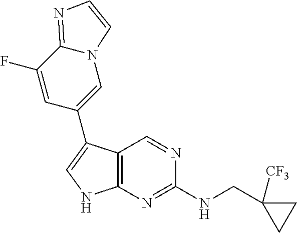 [SMILES: Fc1cc(-c2c[nH]c3nc(NCC4(C(F)(F)F)CC4)ncc23)cn2ccnc12] | 211 |
| 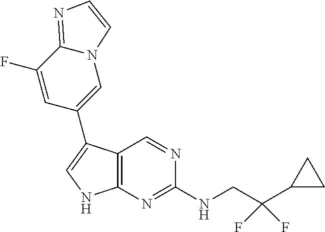 [SMILES: Fc1cc(-c2c[nH]c3nc(NCC(F)(F)C4CC4)ncc23)cn2ccnc12] | 212 |
| 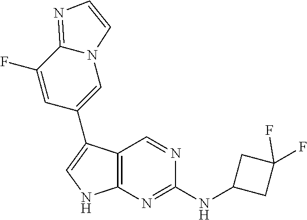 [SMILES: Fc1cc(-c2c[nH]c3nc(NC4CC(F)(F)C4)ncc23)cn2ccnc12] | 213 |
| 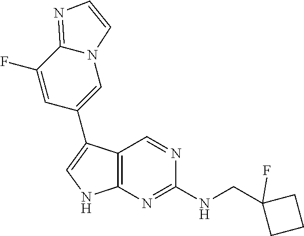 [SMILES: Fc1cc(-c2c[nH]c3nc(NCC4(F)CCC4)ncc23)cn2ccnc12] | 214 |
| 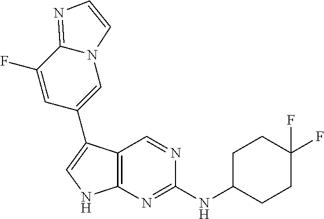 [SMILES: Fc1cc(-c2c[nH]c3nc(NC4CCC(F)(F)CC4)ncc23)cn2ccnc12] | 215 |
| 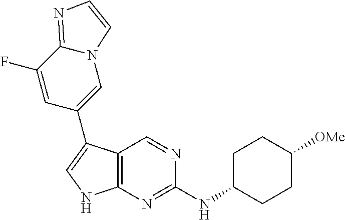 [SMILES: CO[C@H]1CC[C@@H](Nc2ncc3c(-c4cc(F)c5nccn5c4)c[nH]c3n2)CC1] | 216 |
| 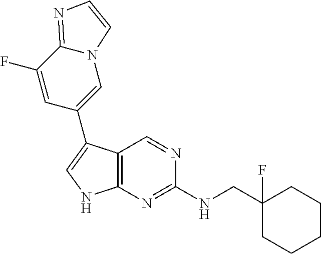 [SMILES: Fc1cc(-c2c[nH]c3nc(NCC4(F)CCCCC4)ncc23)cn2ccnc12] | 217 |
| 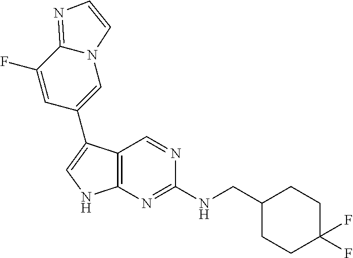 [SMILES: Fc1cc(-c2c[nH]c3nc(NCC4CCC(F)(F)CC4)ncc23)cn2ccnc12] | 218 |
| 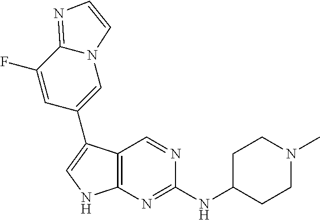 [SMILES: CN1CCC(Nc2ncc3c(-c4cc(F)c5nccn5c4)c[nH]c3n2)CC1] | 219 |
| 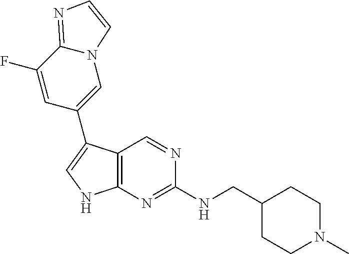 [SMILES: CN1CCC(CNc2ncc3c(-c4cc(F)c5nccn5c4)c[nH]c3n2)CC1] | 220 |
| 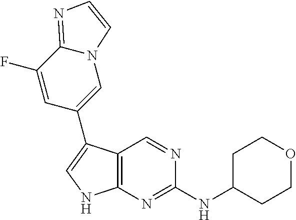 [SMILES: Fc1cc(-c2c[nH]c3nc(NC4CCOCC4)ncc23)cn2ccnc12] | 221 |
| 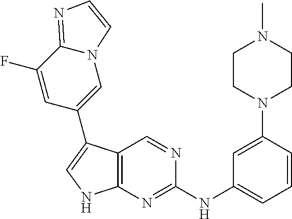 [SMILES: CN1CCN(c2cccc(Nc3ncc4c(-c5cc(F)c6nccn6c5)c[nH]c4n3)c2)CC1] | 222 |
| 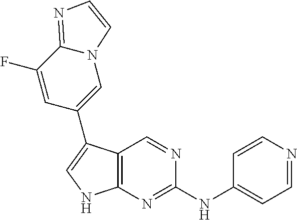 [SMILES: Fc1cc(-c2c[nH]c3nc(Nc4ccncc4)ncc23)cn2ccnc12] | 223 |
| 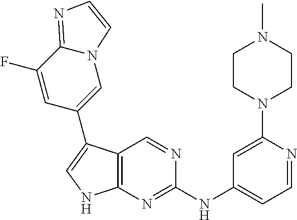 [SMILES: CN1CCN(c2cc(Nc3ncc4c(-c5cc(F)c6nccn6c5)c[nH]c4n3)ccn2)CC1] | 224 |
| 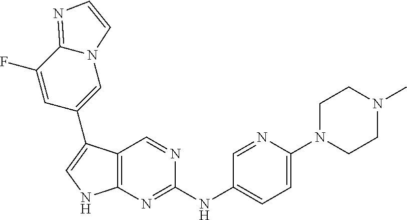 [SMILES: CN1CCN(c2ccc(Nc3ncc4c(-c5cc(F)c6nccn6c5)c[nH]c4n3)cn2)CC1] | 225 |
| 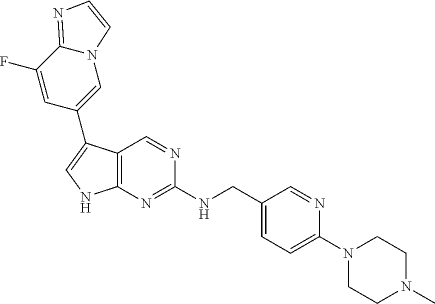 [SMILES: CN1CCN(c2ccc(CNc3ncc4c(-c5cc(F)c6nccn6c5)c[nH]c4n3)cn2)CC1] | 226 |
| 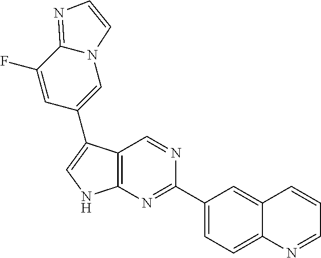 [SMILES: Fc1cc(-c2c[nH]c3nc(-c4ccc5ncccc5c4)ncc23)cn2ccnc12] | 227 |
| 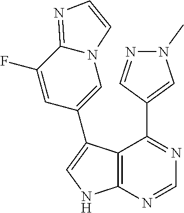 [SMILES: Cn1cc(-c2ncnc3[nH]cc(-c4cc(F)c5nccn5c4)c23)cn1] | 228 |
| 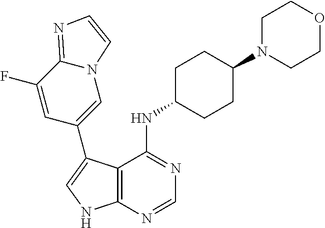 [SMILES: Fc1cc(-c2c[nH]c3ncnc(N[C@H]4CC[C@H](N5CCOCC5)CC4)c23)cn2ccnc12] | 229 |
| 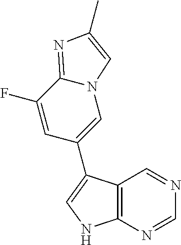 [SMILES: Cc1cn2cc(-c3c[nH]c4ncncc34)cc(F)c2n1] | 230 |
| 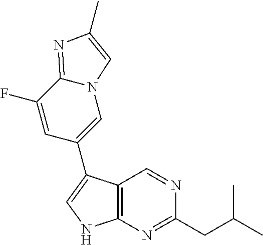 [SMILES: Cc1cn2cc(-c3c[nH]c4nc(CC(C)C)ncc34)cc(F)c2n1] | 231 |
| 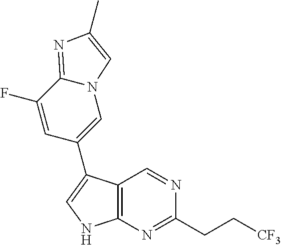 [SMILES: Cc1cn2cc(-c3c[nH]c4nc(CCC(F)(F)F)ncc34)cc(F)c2n1] | 232 |
| 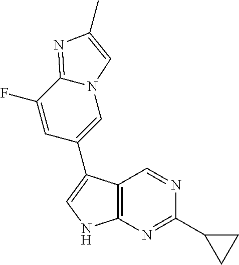 [SMILES: Cc1cn2cc(-c3c[nH]c4nc(C5CC5)ncc34)cc(F)c2n1] | 233 |
| 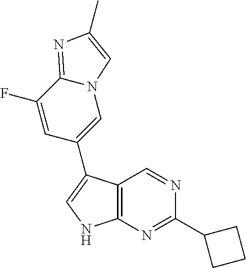 [SMILES: Cc1cn2cc(-c3c[nH]c4nc(C5CCC5)ncc34)cc(F)c2n1] | 234 |
| 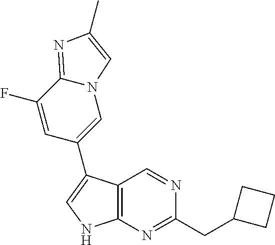 [SMILES: Cc1cn2cc(-c3c[nH]c4nc(CC5CCC5)ncc34)cc(F)c2n1] | 235 |
| 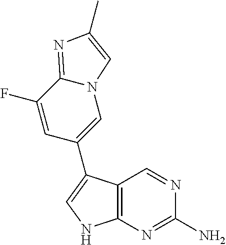 [SMILES: Cc1cn2cc(-c3c[nH]c4nc(N)ncc34)cc(F)c2n1] | 236 |
| 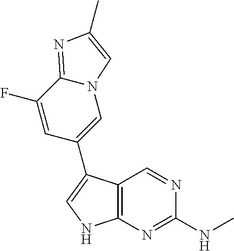 [SMILES: CNc1ncc2c(-c3cc(F)c4nc(C)cn4c3)c[nH]c2n1] | 237 |
| 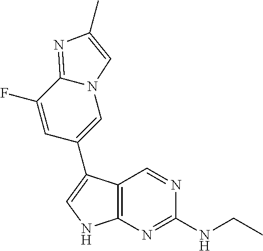 [SMILES: CCNc1ncc2c(-c3cc(F)c4nc(C)cn4c3)c[nH]c2n1] | 238 |
| 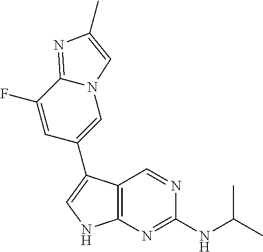 [SMILES: Cc1cn2cc(-c3c[nH]c4nc(NC(C)C)ncc34)cc(F)c2n1] | 239 |
| 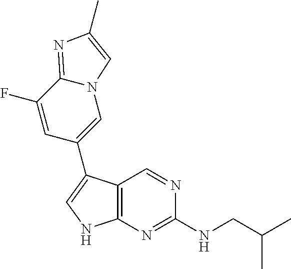 [SMILES: Cc1cn2cc(-c3c[nH]c4nc(NCC(C)C)ncc34)cc(F)c2n1] | 240 |
| 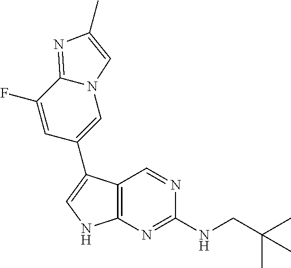 [SMILES: Cc1cn2cc(-c3c[nH]c4nc(NCC(C)(C)C)ncc34)cc(F)c2n1] | 241 |
| 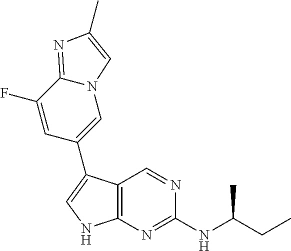 [SMILES: CC[C@H](C)Nc1ncc2c(-c3cc(F)c4nc(C)cn4c3)c[nH]c2n1] | 242 |
| 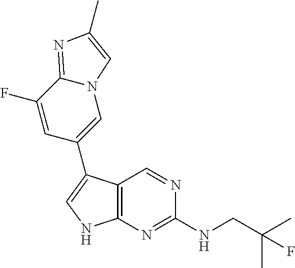 [SMILES: Cc1cn2cc(-c3c[nH]c4nc(NCC(C)(C)F)ncc34)cc(F)c2n1] | 243 |
| 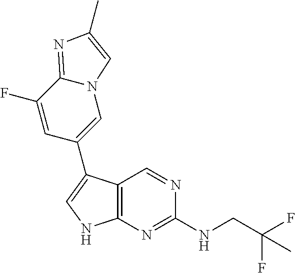 [SMILES: Cc1cn2cc(-c3c[nH]c4nc(NCC(C)(F)F)ncc34)cc(F)c2n1] | 244 |
| 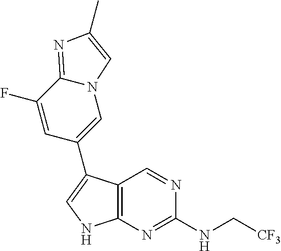 [SMILES: Cc1cn2cc(-c3c[nH]c4nc(NCC(F)(F)F)ncc34)cc(F)c2n1] | 245 |
| 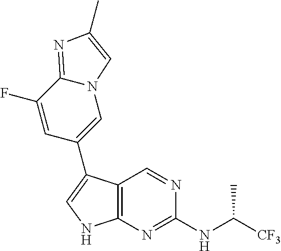 [SMILES: Cc1cn2cc(-c3c[nH]c4nc(N[C@H](C)C(F)(F)F)ncc34)cc(F)c2n1] | 246 |
| 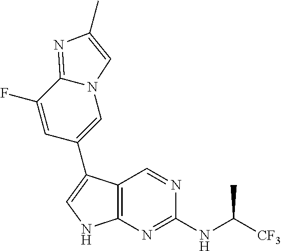 [SMILES: Cc1cn2cc(-c3c[nH]c4nc(N[C@@H](C)C(F)(F)F)ncc34)cc(F)c2n1] | 247 |
| 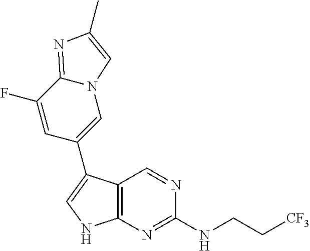 [SMILES: Cc1cn2cc(-c3c[nH]c4nc(NCCC(F)(F)F)ncc34)cc(F)c2n1] | 248 |
| 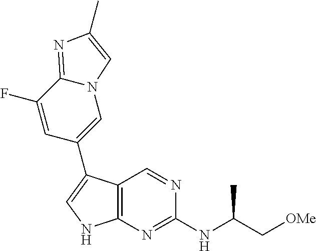 [SMILES: COC[C@H](C)Nc1ncc2c(-c3cc(F)c4nc(C)cn4c3)c[nH]c2n1] | 249 |
| 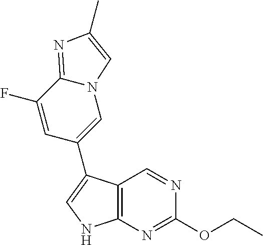 [SMILES: CCOc1ncc2c(-c3cc(F)c4nc(C)cn4c3)c[nH]c2n1] | 250 |
| 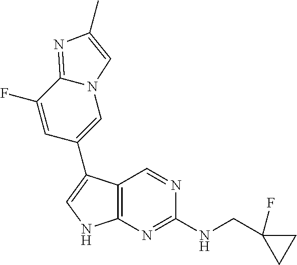 [SMILES: Cc1cn2cc(-c3c[nH]c4nc(NCC5(F)CC5)ncc34)cc(F)c2n1] | 251 |
| 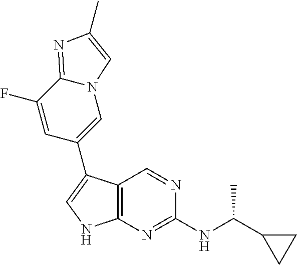 [SMILES: Cc1cn2cc(-c3c[nH]c4nc(N[C@H](C)C5CC5)ncc34)cc(F)c2n1] | 252 |
| 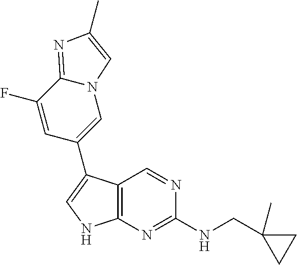 [SMILES: Cc1cn2cc(-c3c[nH]c4nc(NCC5(C)CC5)ncc34)cc(F)c2n1] | 253 |
| 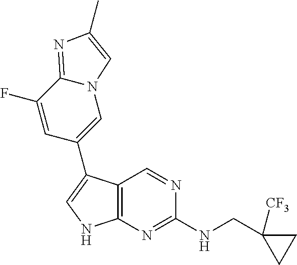 [SMILES: Cc1cn2cc(-c3c[nH]c4nc(NCC5(C(F)(F)F)CC5)ncc34)cc(F)c2n1] | 254 |
| 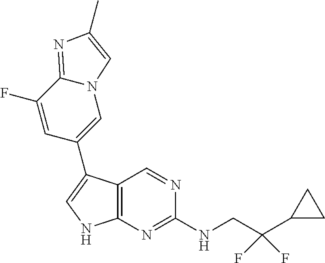 [SMILES: Cc1cn2cc(-c3c[nH]c4nc(NCC(F)(F)C5CC5)ncc34)cc(F)c2n1] | 255 |
| 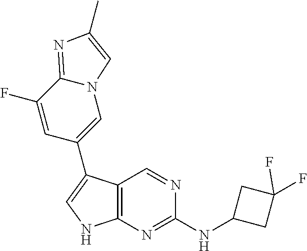 [SMILES: Cc1cn2cc(-c3c[nH]c4nc(NC5CC(F)(F)C5)ncc34)cc(F)c2n1] | 256 |
| 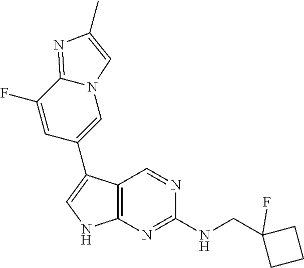 [SMILES: Cc1cn2cc(-c3c[nH]c4nc(NCC5(F)CCC5)ncc34)cc(F)c2n1] | 257 |
| 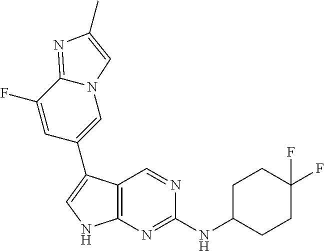 [SMILES: Cc1cn2cc(-c3c[nH]c4nc(NC5CCC(F)(F)CC5)ncc34)cc(F)c2n1] | 258 |
| 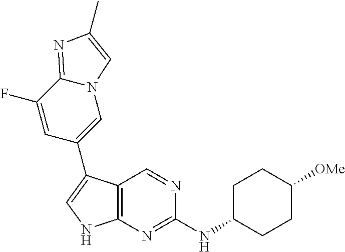 [SMILES: CO[C@H]1CC[C@@H](Nc2ncc3c(-c4cc(F)c5nc(C)cn5c4)c[nH]c3n2)CC1] | 259 |
| 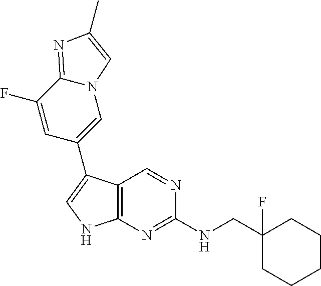 [SMILES: Cc1cn2cc(-c3c[nH]c4nc(NCC5(F)CCCCC5)ncc34)cc(F)c2n1] | 260 |
| 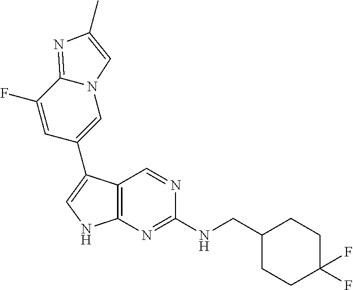 [SMILES: Cc1cn2cc(-c3c[nH]c4nc(NCC5CCC(F)(F)CC5)ncc34)cc(F)c2n1] | 261 |
| 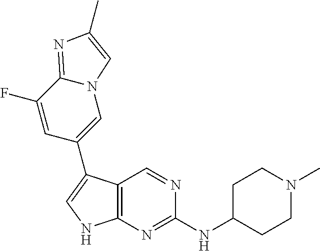 [SMILES: Cc1cn2cc(-c3c[nH]c4nc(NC5CCN(C)CC5)ncc34)cc(F)c2n1] | 262 |
| 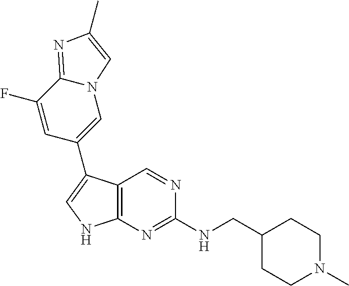 [SMILES: Cc1cn2cc(-c3c[nH]c4nc(NCC5CCN(C)CC5)ncc34)cc(F)c2n1] | 263 |
| 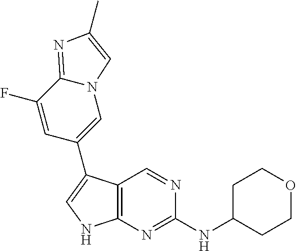 [SMILES: Cc1cn2cc(-c3c[nH]c4nc(NC5CCOCC5)ncc34)cc(F)c2n1] | 264 |
| 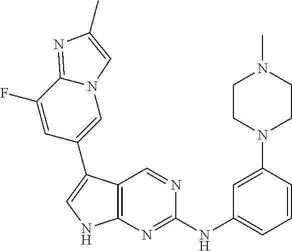 [SMILES: Cc1cn2cc(-c3c[nH]c4nc(Nc5cccc(N6CCN(C)CC6)c5)ncc34)cc(F)c2n1] | 265 |
| 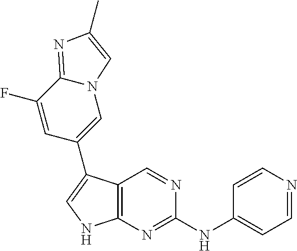 [SMILES: Cc1cn2cc(-c3c[nH]c4nc(Nc5ccncc5)ncc34)cc(F)c2n1] | 266 |
| 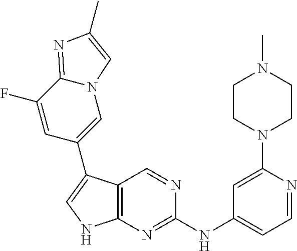 [SMILES: Cc1cn2cc(-c3c[nH]c4nc(Nc5ccnc(N6CCN(C)CC6)c5)ncc34)cc(F)c2n1] | 267 |
| 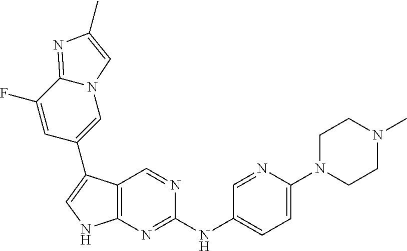 [SMILES: Cc1cn2cc(-c3c[nH]c4nc(Nc5ccc(N6CCN(C)CC6)nc5)ncc34)cc(F)c2n1] | 268 |
| 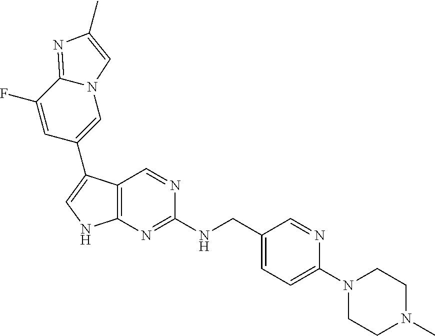 [SMILES: Cc1cn2cc(-c3c[nH]c4nc(NCc5ccc(N6CCN(C)CC6)nc5)ncc34)cc(F)c2n1] | 269 |
| 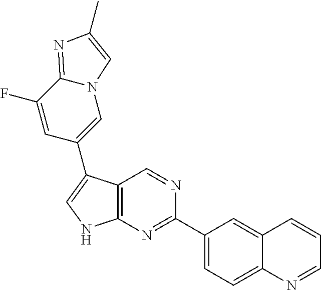 [SMILES: Cc1cn2cc(-c3c[nH]c4nc(-c5ccc6ncccc6c5)ncc34)cc(F)c2n1] | 270 |
| 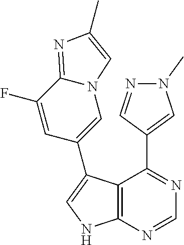 [SMILES: Cc1cn2cc(-c3c[nH]c4ncnc(-c5cnn(C)c5)c34)cc(F)c2n1] | 271 |
| 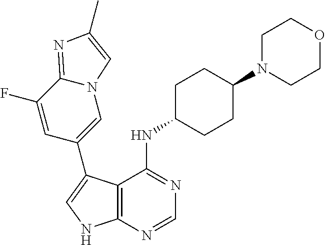 [SMILES: Cc1cn2cc(-c3c[nH]c4ncnc(N[C@H]5CC[C@H](N6CCOCC6)CC5)c34)cc(F)c2n1] | 272 |
| 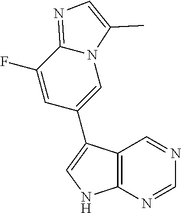 [SMILES: Cc1cnc2c(F)cc(-c3c[nH]c4ncncc34)cn12] | 273 |
| 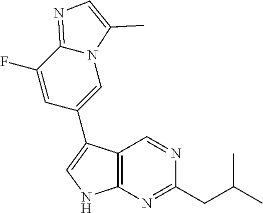 [SMILES: Cc1cnc2c(F)cc(-c3c[nH]c4nc(CC(C)C)ncc34)cn12] | 274 |
| 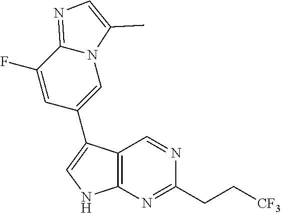 [SMILES: Cc1cnc2c(F)cc(-c3c[nH]c4nc(CCC(F)(F)F)ncc34)cn12] | 275 |
| 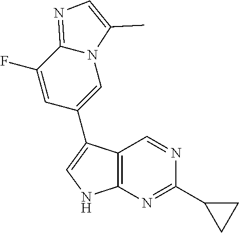 [SMILES: Cc1cnc2c(F)cc(-c3c[nH]c4nc(C5CC5)ncc34)cn12] | 276 |
| 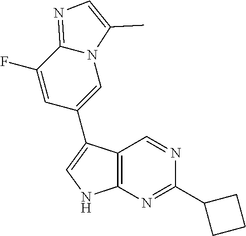 [SMILES: Cc1cnc2c(F)cc(-c3c[nH]c4nc(C5CCC5)ncc34)cn12] | 277 |
| 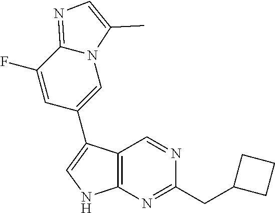 [SMILES: Cc1cnc2c(F)cc(-c3c[nH]c4nc(CC5CCC5)ncc34)cn12] | 278 |
| 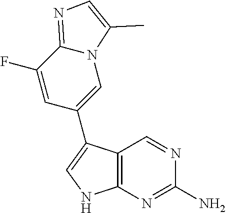 [SMILES: Cc1cnc2c(F)cc(-c3c[nH]c4nc(N)ncc34)cn12] | 279 |
| 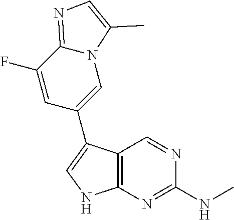 [SMILES: CNc1ncc2c(-c3cc(F)c4ncc(C)n4c3)c[nH]c2n1] | 280 |
| 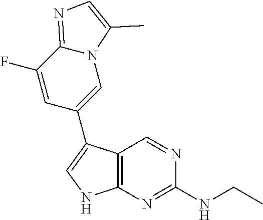 [SMILES: CCNc1ncc2c(-c3cc(F)c4ncc(C)n4c3)c[nH]c2n1] | 281 |
| 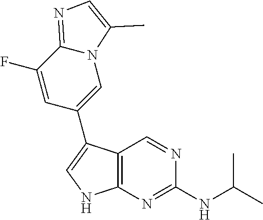 [SMILES: Cc1cnc2c(F)cc(-c3c[nH]c4nc(NC(C)C)ncc34)cn12] | 282 |
| 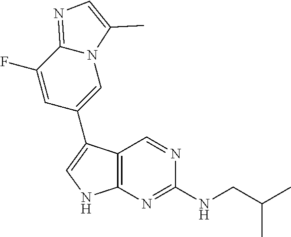 [SMILES: Cc1cnc2c(F)cc(-c3c[nH]c4nc(NCC(C)C)ncc34)cn12] | 283 |
| 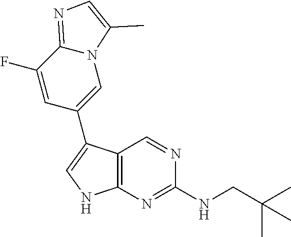 [SMILES: Cc1cnc2c(F)cc(-c3c[nH]c4nc(NCC(C)(C)C)ncc34)cn12] | 284 |
| 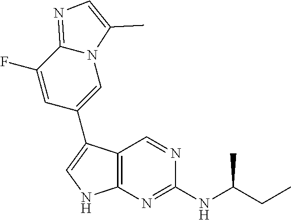 [SMILES: CC[C@H](C)Nc1ncc2c(-c3cc(F)c4ncc(C)n4c3)c[nH]c2n1] | 285 |
| 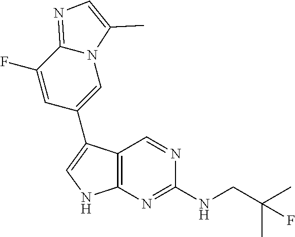 [SMILES: Cc1cnc2c(F)cc(-c3c[nH]c4nc(NCC(C)(C)F)ncc34)cn12] | 286 |
| 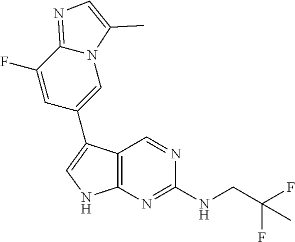 [SMILES: Cc1cnc2c(F)cc(-c3c[nH]c4nc(NCC(C)(F)F)ncc34)cn12] | 287 |
| 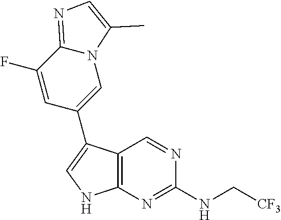 [SMILES: Cc1cnc2c(F)cc(-c3c[nH]c4nc(NCC(F)(F)F)ncc34)cn12] | 288 |
| 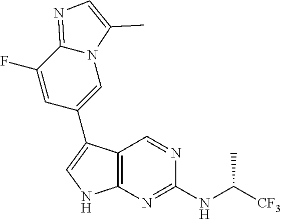 [SMILES: Cc1cnc2c(F)cc(-c3c[nH]c4nc(N[C@H](C)C(F)(F)F)ncc34)cn12] | 289 |
| 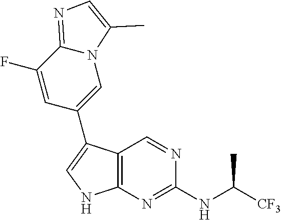 [SMILES: Cc1cnc2c(F)cc(-c3c[nH]c4nc(N[C@@H](C)C(F)(F)F)ncc34)cn12] | 290 |
| 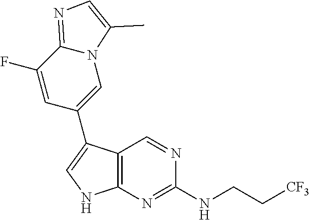 [SMILES: Cc1cnc2c(F)cc(-c3c[nH]c4nc(NCCC(F)(F)F)ncc34)cn12] | 291 |
| 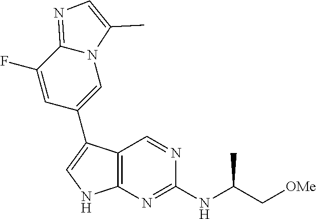 [SMILES: COC[C@H](C)Nc1ncc2c(-c3cc(F)c4ncc(C)n4c3)c[nH]c2n1] | 292 |
| 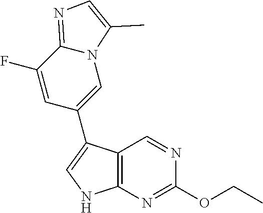 [SMILES: CCOc1ncc2c(-c3cc(F)c4ncc(C)n4c3)c[nH]c2n1] | 293 |
| 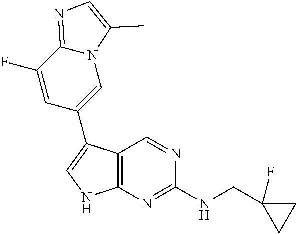 [SMILES: Cc1cnc2c(F)cc(-c3c[nH]c4nc(NCC5(F)CC5)ncc34)cn12] | 294 |
| 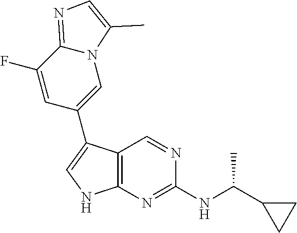 [SMILES: Cc1cnc2c(F)cc(-c3c[nH]c4nc(N[C@H](C)C5CC5)ncc34)cn12] | 295 |
| 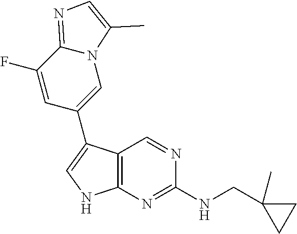 [SMILES: Cc1cnc2c(F)cc(-c3c[nH]c4nc(NCC5(C)CC5)ncc34)cn12] | 296 |
| 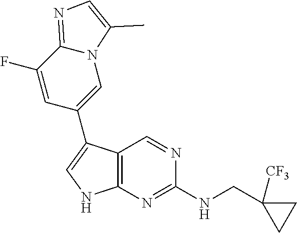 [SMILES: Cc1cnc2c(F)cc(-c3c[nH]c4nc(NCC5(C(F)(F)F)CC5)ncc34)cn12] | 297 |
| 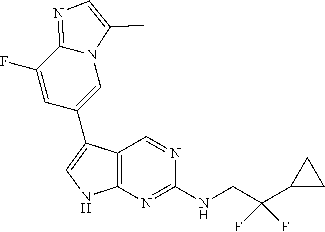 [SMILES: Cc1cnc2c(F)cc(-c3c[nH]c4nc(NCC(F)(F)C5CC5)ncc34)cn12] | 298 |
| 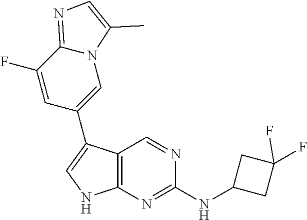 [SMILES: Cc1cnc2c(F)cc(-c3c[nH]c4nc(NC5CC(F)(F)C5)ncc34)cn12] | 299 |
| 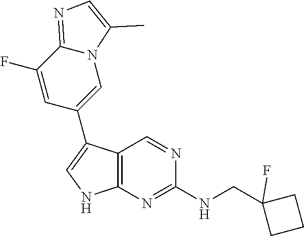 [SMILES: Cc1cnc2c(F)cc(-c3c[nH]c4nc(NCC5(F)CCC5)ncc34)cn12] | 300 |
| 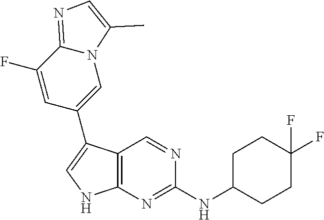 [SMILES: Cc1cnc2c(F)cc(-c3c[nH]c4nc(NC5CCC(F)(F)CC5)ncc34)cn12] | 301 |
| 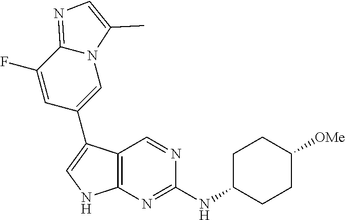 [SMILES: CO[C@H]1CC[C@@H](Nc2ncc3c(-c4cc(F)c5ncc(C)n5c4)c[nH]c3n2)CC1] | 302 |
| 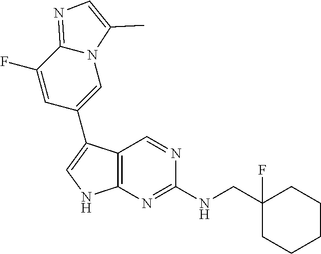 [SMILES: Cc1cnc2c(F)cc(-c3c[nH]c4nc(NCC5(F)CCCCC5)ncc34)cn12] | 303 |
| 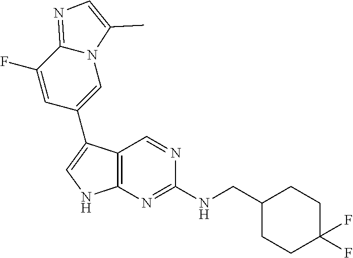 [SMILES: Cc1cnc2c(F)cc(-c3c[nH]c4nc(NCC5CCC(F)(F)CC5)ncc34)cn12] | 304 |
| 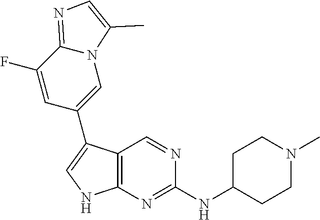 [SMILES: Cc1cnc2c(F)cc(-c3c[nH]c4nc(NC5CCN(C)CC5)ncc34)cn12] | 305 |
| 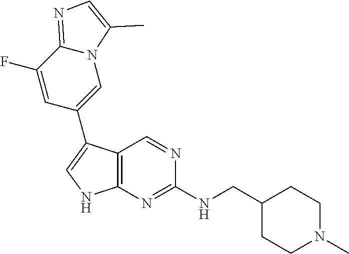 [SMILES: Cc1cnc2c(F)cc(-c3c[nH]c4nc(NCC5CCN(C)CC5)ncc34)cn12] | 306 |
| 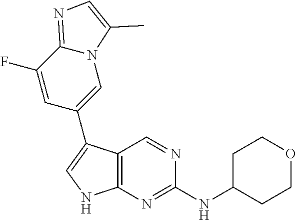 [SMILES: Cc1cnc2c(F)cc(-c3c[nH]c4nc(NC5CCOCC5)ncc34)cn12] | 307 |
| 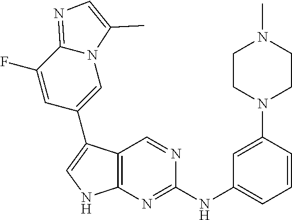 [SMILES: Cc1cnc2c(F)cc(-c3c[nH]c4nc(Nc5cccc(N6CCN(C)CC6)c5)ncc34)cn12] | 308 |
| 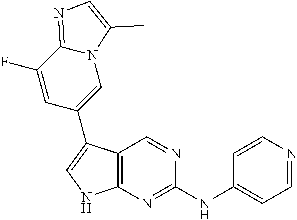 [SMILES: Cc1cnc2c(F)cc(-c3c[nH]c4nc(Nc5ccncc5)ncc34)cn12] | 309 |
| 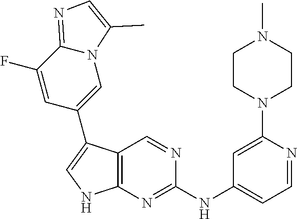 [SMILES: Cc1cnc2c(F)cc(-c3c[nH]c4nc(Nc5ccnc(N6CCN(C)CC6)c5)ncc34)cn12] | 310 |
| 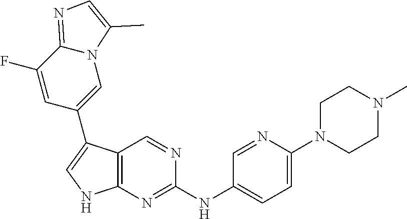 [SMILES: Cc1cnc2c(F)cc(-c3c[nH]c4nc(Nc5ccc(N6CCN(C)CC6)nc5)ncc34)cn12] | 311 |
| 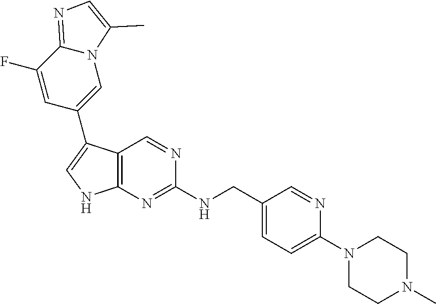 [SMILES: Cc1cnc2c(F)cc(-c3c[nH]c4nc(NCc5ccc(N6CCN(C)CC6)nc5)ncc34)cn12] | 312 |
| 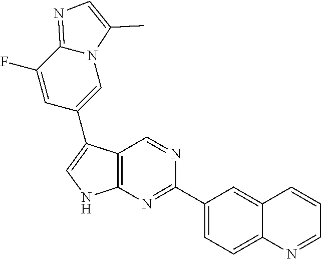 [SMILES: Cc1cnc2c(F)cc(-c3c[nH]c4nc(-c5ccc6ncccc6c5)ncc34)cn12] | 313 |
| 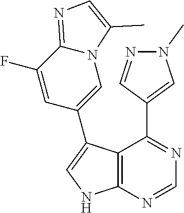 [SMILES: Cc1cnc2c(F)cc(-c3c[nH]c4ncnc(-c5cnn(C)c5)c34)cn12] | 314 |
| 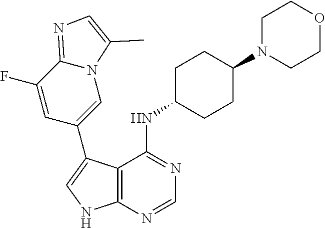 [SMILES: Cc1cnc2c(F)cc(-c3c[nH]c4ncnc(N[C@H]5CC[C@H](N6CCOCC6)CC5)c34)cn12] | 315 |
| 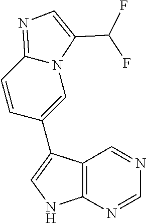 [SMILES: FC(F)c1cnc2ccc(-c3c[nH]c4ncncc34)cn12] | 316 |
| 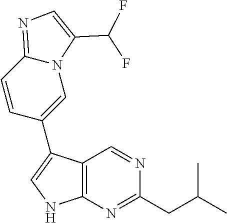 [SMILES: CC(C)Cc1ncc2c(-c3ccc4ncc(C(F)F)n4c3)c[nH]c2n1] | 317 |
| 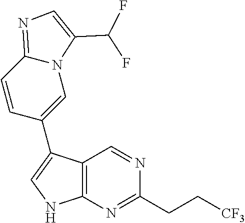 [SMILES: FC(F)c1cnc2ccc(-c3c[nH]c4nc(CCC(F)(F)F)ncc34)cn12] | 318 |
| 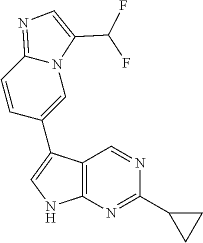 [SMILES: FC(F)c1cnc2ccc(-c3c[nH]c4nc(C5CC5)ncc34)cn12] | 319 |
| 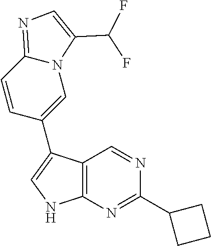 [SMILES: FC(F)c1cnc2ccc(-c3c[nH]c4nc(C5CCC5)ncc34)cn12] | 320 |
| 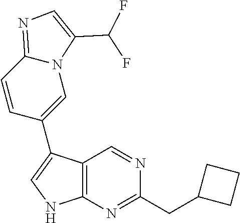 [SMILES: FC(F)c1cnc2ccc(-c3c[nH]c4nc(CC5CCC5)ncc34)cn12] | 321 |
| 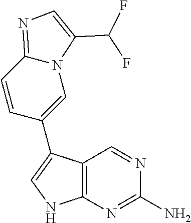 [SMILES: Nc1ncc2c(-c3ccc4ncc(C(F)F)n4c3)c[nH]c2n1] | 322 |
| 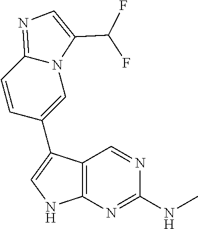 [SMILES: CNc1ncc2c(-c3ccc4ncc(C(F)F)n4c3)c[nH]c2n1] | 323 |
| 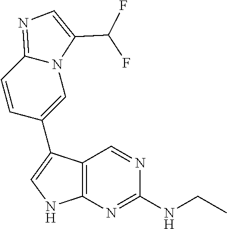 [SMILES: CCNc1ncc2c(-c3ccc4ncc(C(F)F)n4c3)c[nH]c2n1] | 324 |
| 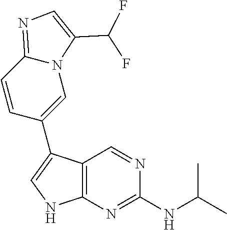 [SMILES: CC(C)Nc1ncc2c(-c3ccc4ncc(C(F)F)n4c3)c[nH]c2n1] | 325 |
| 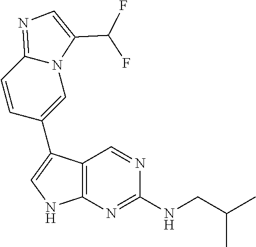 [SMILES: CC(C)CNc1ncc2c(-c3ccc4ncc(C(F)F)n4c3)c[nH]c2n1] | 326 |
| 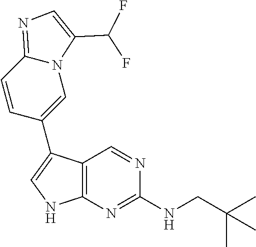 [SMILES: CC(C)(C)CNc1ncc2c(-c3ccc4ncc(C(F)F)n4c3)c[nH]c2n1] | 327 |
| 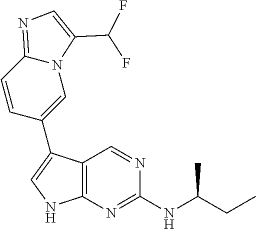 [SMILES: CC[C@H](C)Nc1ncc2c(-c3ccc4ncc(C(F)F)n4c3)c[nH]c2n1] | 328 |
| 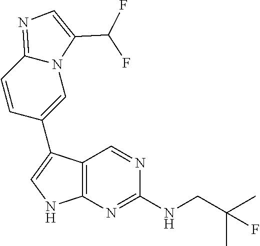 [SMILES: CC(C)(F)CNc1ncc2c(-c3ccc4ncc(C(F)F)n4c3)c[nH]c2n1] | 329 |
| 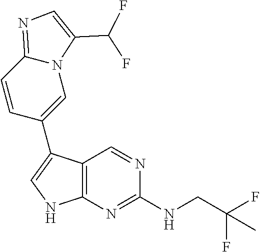 [SMILES: CC(F)(F)CNc1ncc2c(-c3ccc4ncc(C(F)F)n4c3)c[nH]c2n1] | 330 |
| 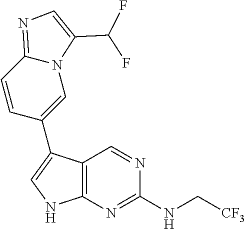 [SMILES: FC(F)c1cnc2ccc(-c3c[nH]c4nc(NCC(F)(F)F)ncc34)cn12] | 331 |
| 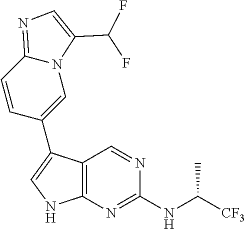 [SMILES: C[C@@H](Nc1ncc2c(-c3ccc4ncc(C(F)F)n4c3)c[nH]c2n1)C(F)(F)F] | 332 |
| 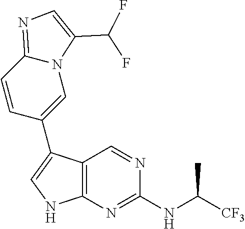 [SMILES: C[C@H](Nc1ncc2c(-c3ccc4ncc(C(F)F)n4c3)c[nH]c2n1)C(F)(F)F] | 333 |
| 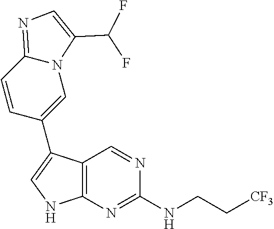 [SMILES: FC(F)c1cnc2ccc(-c3c[nH]c4nc(NCCC(F)(F)F)ncc34)cn12] | 334 |
| 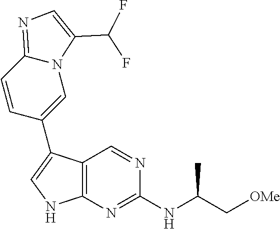 [SMILES: COC[C@H](C)Nc1ncc2c(-c3ccc4ncc(C(F)F)n4c3)c[nH]c2n1] | 335 |
| 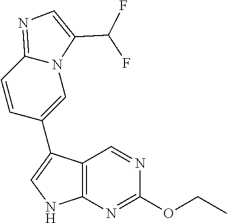 [SMILES: CCOc1ncc2c(-c3ccc4ncc(C(F)F)n4c3)c[nH]c2n1] | 336 |
| 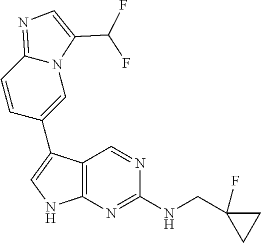 [SMILES: FC(F)c1cnc2ccc(-c3c[nH]c4nc(NCC5(F)CC5)ncc34)cn12] | 337 |
| 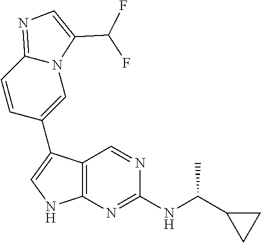 [SMILES: C[C@@H](Nc1ncc2c(-c3ccc4ncc(C(F)F)n4c3)c[nH]c2n1)C1CC1] | 338 |
| 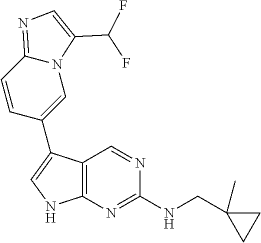 [SMILES: CC1(CNc2ncc3c(-c4ccc5ncc(C(F)F)n5c4)c[nH]c3n2)CC1] | 339 |
| 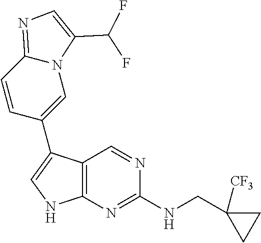 [SMILES: FC(F)c1cnc2ccc(-c3c[nH]c4nc(NCC5(C(F)(F)F)CC5)ncc34)cn12] | 340 |
| 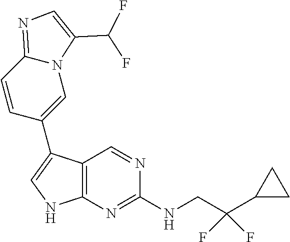 [SMILES: FC(F)c1cnc2ccc(-c3c[nH]c4nc(NCC(F)(F)C5CC5)ncc34)cn12] | 341 |
| 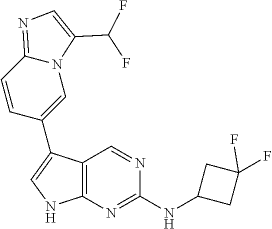 [SMILES: FC(F)c1cnc2ccc(-c3c[nH]c4nc(NC5CC(F)(F)C5)ncc34)cn12] | 342 |
| 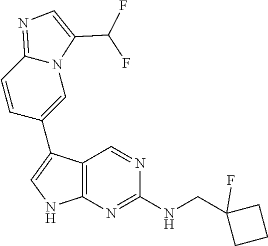 [SMILES: FC(F)c1cnc2ccc(-c3c[nH]c4nc(NCC5(F)CCC5)ncc34)cn12] | 343 |
| 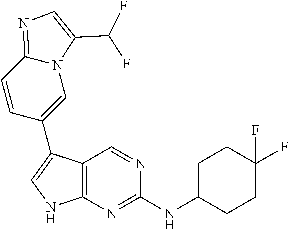 [SMILES: FC(F)c1cnc2ccc(-c3c[nH]c4nc(NC5CCC(F)(F)CC5)ncc34)cn12] | 344 |
| 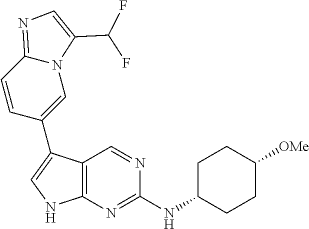 [SMILES: CO[C@H]1CC[C@@H](Nc2ncc3c(-c4ccc5ncc(C(F)F)n5c4)c[nH]c3n2)CC1] | 345 |
| 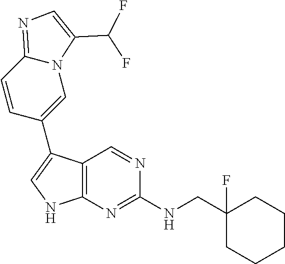 [SMILES: FC(F)c1cnc2ccc(-c3c[nH]c4nc(NCC5(F)CCCCC5)ncc34)cn12] | 346 |
| 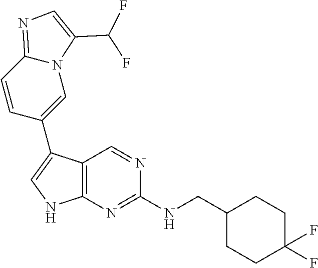 [SMILES: FC(F)c1cnc2ccc(-c3c[nH]c4nc(NCC5CCC(F)(F)CC5)ncc34)cn12] | 347 |
| 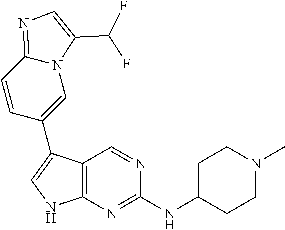 [SMILES: CN1CCC(Nc2ncc3c(-c4ccc5ncc(C(F)F)n5c4)c[nH]c3n2)CC1] | 348 |
| 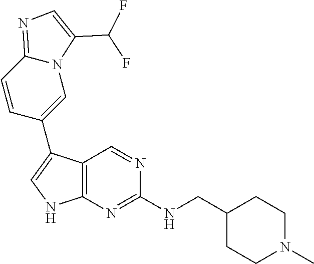 [SMILES: CN1CCC(CNc2ncc3c(-c4ccc5ncc(C(F)F)n5c4)c[nH]c3n2)CC1] | 349 |
| 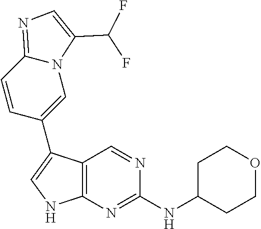 [SMILES: FC(F)c1cnc2ccc(-c3c[nH]c4nc(NC5CCOCC5)ncc34)cn12] | 350 |
| 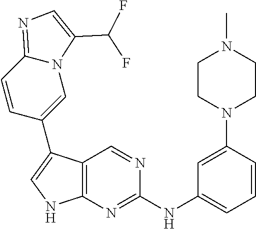 [SMILES: CN1CCN(c2cccc(Nc3ncc4c(-c5ccc6ncc(C(F)F)n6c5)c[nH]c4n3)c2)CC1] | 351 |
| 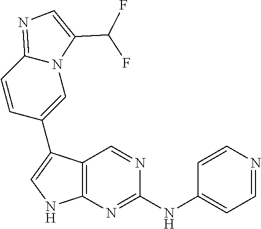 [SMILES: FC(F)c1cnc2ccc(-c3c[nH]c4nc(Nc5ccncc5)ncc34)cn12] | 352 |
| 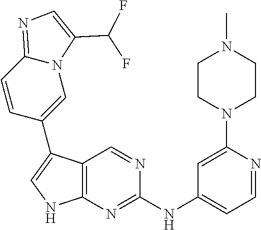 [SMILES: CN1CCN(c2cc(Nc3ncc4c(-c5ccc6ncc(C(F)F)n6c5)c[nH]c4n3)ccn2)CC1] | 353 |
| 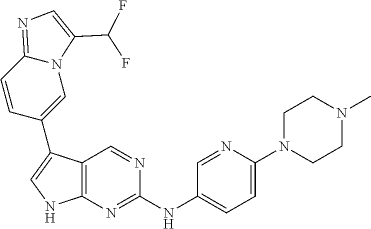 [SMILES: CN1CCN(c2ccc(Nc3ncc4c(-c5ccc6ncc(C(F)F)n6c5)c[nH]c4n3)cn2)CC1] | 354 |
| 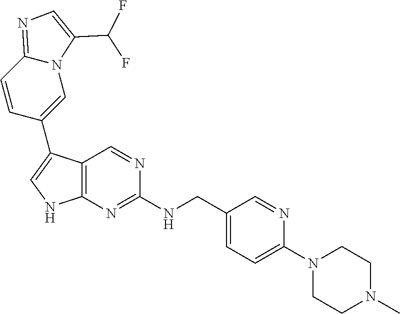 [SMILES: CN1CCN(c2ccc(CNc3ncc4c(-c5ccc6ncc(C(F)F)n6c5)c[nH]c4n3)cn2)CC1] | 355 |
| 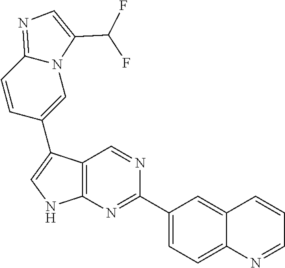 [SMILES: FC(F)c1cnc2ccc(-c3c[nH]c4nc(-c5ccc6ncccc6c5)ncc34)cn12] | 356 |
| 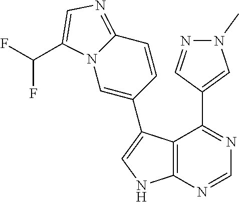 [SMILES: Cn1cc(-c2ncnc3[nH]cc(-c4ccc5ncc(C(F)F)n5c4)c23)cn1] | 357 |
| 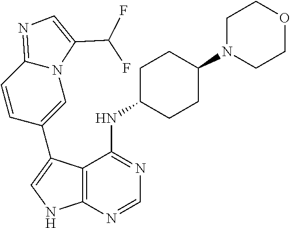 [SMILES: FC(F)c1cnc2ccc(-c3c[nH]c4ncnc(N[C@H]5CC[C@H](N6CCOCC6)CC5)c34)cn12] | 358 |
| 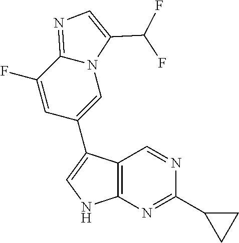 [SMILES: Fc1cc(-c2c[nH]c3nc(C4CC4)ncc23)cn2c(C(F)F)cnc12] | 359 |
| 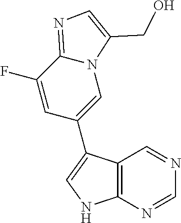 [SMILES: OCc1cnc2c(F)cc(-c3c[nH]c4ncncc34)cn12] | 360 |
| 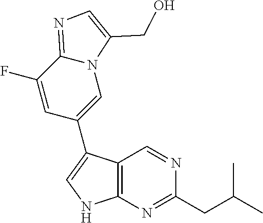 [SMILES: CC(C)Cc1ncc2c(-c3cc(F)c4ncc(CO)n4c3)c[nH]c2n1] | 361 |
| 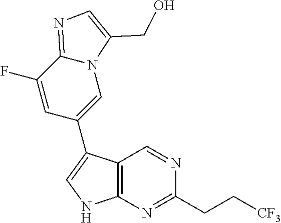 [SMILES: OCc1cnc2c(F)cc(-c3c[nH]c4nc(CCC(F)(F)F)ncc34)cn12] | 362 |
| 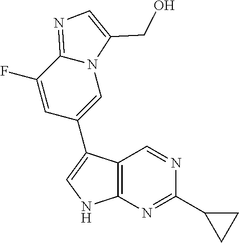 [SMILES: OCc1cnc2c(F)cc(-c3c[nH]c4nc(C5CC5)ncc34)cn12] | 363 |
| 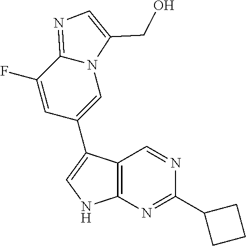 [SMILES: OCc1cnc2c(F)cc(-c3c[nH]c4nc(C5CCC5)ncc34)cn12] | 364 |
| 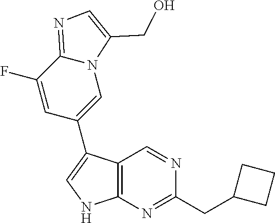 [SMILES: OCc1cnc2c(F)cc(-c3c[nH]c4nc(CC5CCC5)ncc34)cn12] | 365 |
| 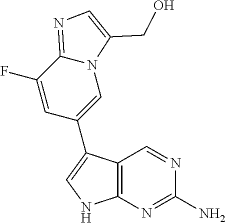 [SMILES: Nc1ncc2c(-c3cc(F)c4ncc(CO)n4c3)c[nH]c2n1] | 366 |
| 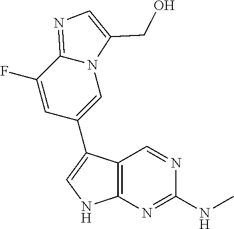 [SMILES: CNc1ncc2c(-c3cc(F)c4ncc(CO)n4c3)c[nH]c2n1] | 367 |
| 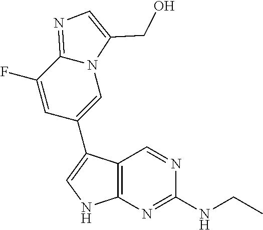 [SMILES: CCNc1ncc2c(-c3cc(F)c4ncc(CO)n4c3)c[nH]c2n1] | 368 |
| 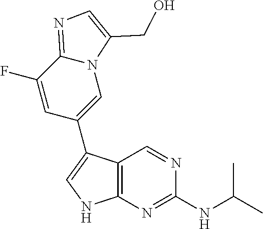 [SMILES: CC(C)Nc1ncc2c(-c3cc(F)c4ncc(CO)n4c3)c[nH]c2n1] | 369 |
| 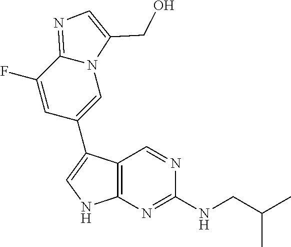 [SMILES: CC(C)CNc1ncc2c(-c3cc(F)c4ncc(CO)n4c3)c[nH]c2n1] | 370 |
| 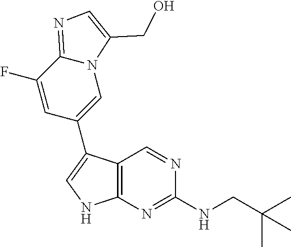 [SMILES: CC(C)(C)CNc1ncc2c(-c3cc(F)c4ncc(CO)n4c3)c[nH]c2n1] | 371 |
| 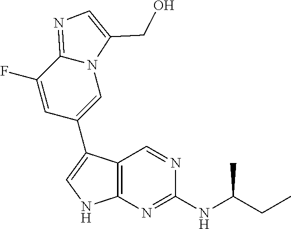 [SMILES: CC[C@H](C)Nc1ncc2c(-c3cc(F)c4ncc(CO)n4c3)c[nH]c2n1] | 372 |
| 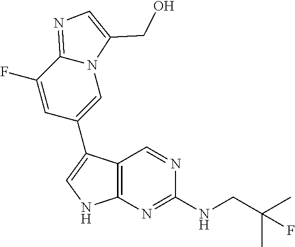 [SMILES: CC(C)(F)CNc1ncc2c(-c3cc(F)c4ncc(CO)n4c3)c[nH]c2n1] | 373 |
| 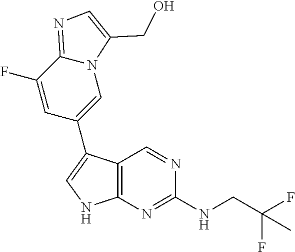 [SMILES: CC(F)(F)CNc1ncc2c(-c3cc(F)c4ncc(CO)n4c3)c[nH]c2n1] | 374 |
| 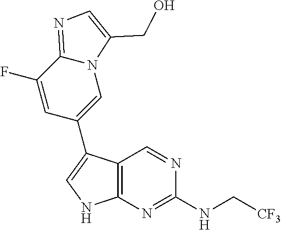 [SMILES: OCc1cnc2c(F)cc(-c3c[nH]c4nc(NCC(F)(F)F)ncc34)cn12] | 375 |
| 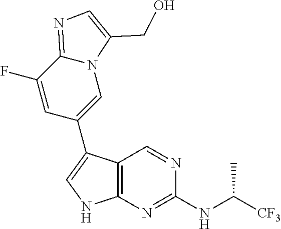 [SMILES: C[C@@H](Nc1ncc2c(-c3cc(F)c4ncc(CO)n4c3)c[nH]c2n1)C(F)(F)F] | 376 |
| 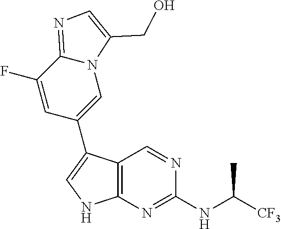 [SMILES: C[C@H](Nc1ncc2c(-c3cc(F)c4ncc(CO)n4c3)c[nH]c2n1)C(F)(F)F] | 377 |
| 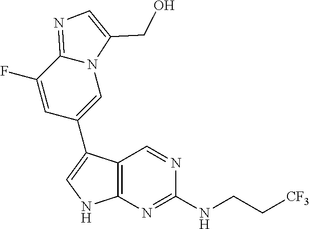 [SMILES: OCc1cnc2c(F)cc(-c3c[nH]c4nc(NCCC(F)(F)F)ncc34)cn12] | 378 |
| 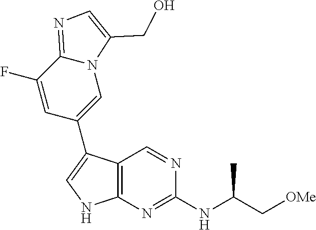 [SMILES: COC[C@H](C)Nc1ncc2c(-c3cc(F)c4ncc(CO)n4c3)c[nH]c2n1] | 379 |
| 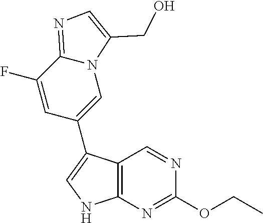 [SMILES: CCOc1ncc2c(-c3cc(F)c4ncc(CO)n4c3)c[nH]c2n1] | 380 |
| 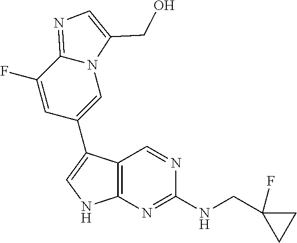 [SMILES: OCc1cnc2c(F)cc(-c3c[nH]c4nc(NCC5(F)CC5)ncc34)cn12] | 381 |
| 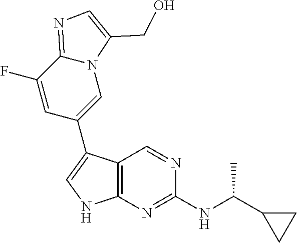 [SMILES: C[C@@H](Nc1ncc2c(-c3cc(F)c4ncc(CO)n4c3)c[nH]c2n1)C1CC1] | 382 |
| 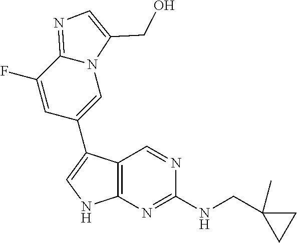 [SMILES: CC1(CNc2ncc3c(-c4cc(F)c5ncc(CO)n5c4)c[nH]c3n2)CC1] | 383 |
| 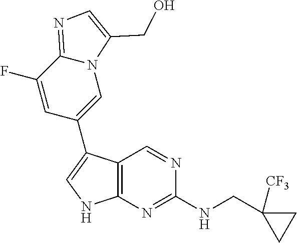 [SMILES: OCc1cnc2c(F)cc(-c3c[nH]c4nc(NCC5(C(F)(F)F)CC5)ncc34)cn12] | 384 |
| 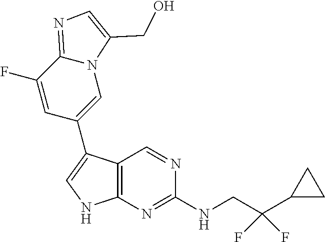 [SMILES: OCc1cnc2c(F)cc(-c3c[nH]c4nc(NCC(F)(F)C5CC5)ncc34)cn12] | 385 |
| 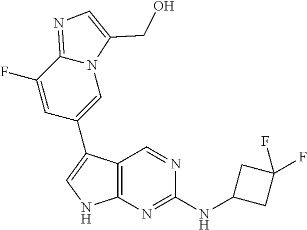 [SMILES: OCc1cnc2c(F)cc(-c3c[nH]c4nc(NC5CC(F)(F)C5)ncc34)cn12] | 386 |
| 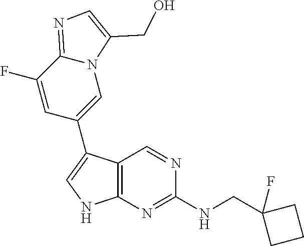 [SMILES: OCc1cnc2c(F)cc(-c3c[nH]c4nc(NCC5(F)CCC5)ncc34)cn12] | 387 |
| 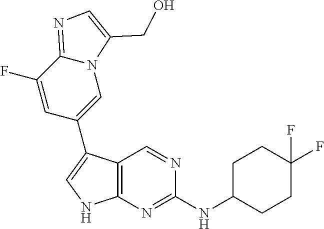 [SMILES: OCc1cnc2c(F)cc(-c3c[nH]c4nc(NC5CCC(F)(F)CC5)ncc34)cn12] | 388 |
| 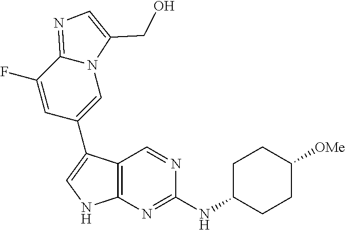 [SMILES: CO[C@H]1CC[C@@H](Nc2ncc3c(-c4cc(F)c5ncc(CO)n5c4)c[nH]c3n2)CC1] | 389 |
| 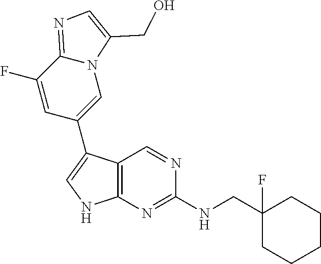 [SMILES: OCc1cnc2c(F)cc(-c3c[nH]c4nc(NCC5(F)CCCCC5)ncc34)cn12] | 390 |
| 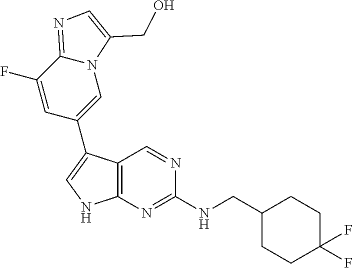 [SMILES: OCc1cnc2c(F)cc(-c3c[nH]c4nc(NCC5CCC(F)(F)CC5)ncc34)cn12] | 391 |
| 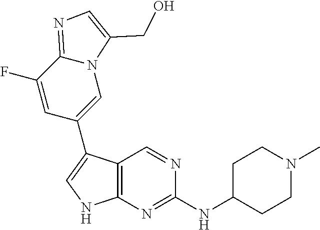 [SMILES: CN1CCC(Nc2ncc3c(-c4cc(F)c5ncc(CO)n5c4)c[nH]c3n2)CC1] | 392 |
| 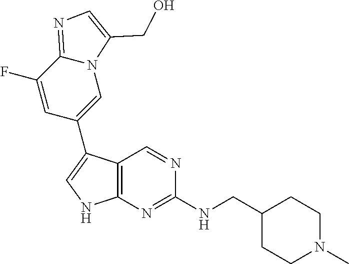 [SMILES: CN1CCC(CNc2ncc3c(-c4cc(F)c5ncc(CO)n5c4)c[nH]c3n2)CC1] | 393 |
| 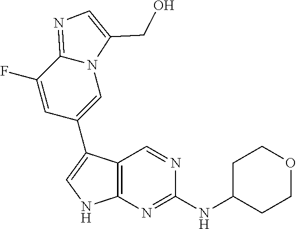 [SMILES: OCc1cnc2c(F)cc(-c3c[nH]c4nc(NC5CCOCC5)ncc34)cn12] | 394 |
| 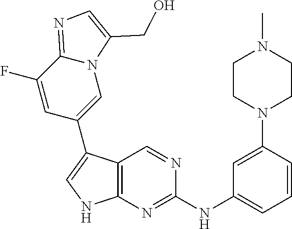 [SMILES: CN1CCN(c2cccc(Nc3ncc4c(-c5cc(F)c6ncc(CO)n6c5)c[nH]c4n3)c2)CC1] | 395 |
| 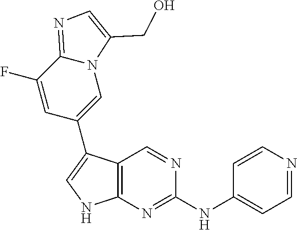 [SMILES: OCc1cnc2c(F)cc(-c3c[nH]c4nc(Nc5ccncc5)ncc34)cn12] | 396 |
| 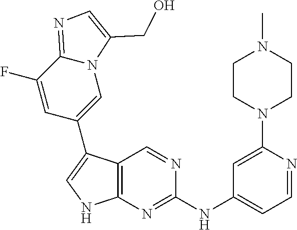 [SMILES: CN1CCN(c2cc(Nc3ncc4c(-c5cc(F)c6ncc(CO)n6c5)c[nH]c4n3)ccn2)CC1] | 397 |
| 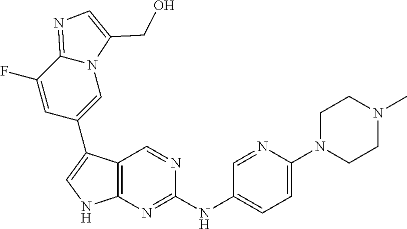 [SMILES: CN1CCN(c2ccc(Nc3ncc4c(-c5cc(F)c6ncc(CO)n6c5)c[nH]c4n3)cn2)CC1] | 398 |
| 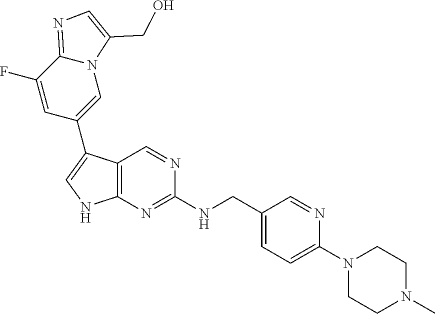 [SMILES: CN1CCN(c2ccc(CNc3ncc4c(-c5cc(F)c6ncc(CO)n6c5)c[nH]c4n3)cn2)CC1] | 399 |
| 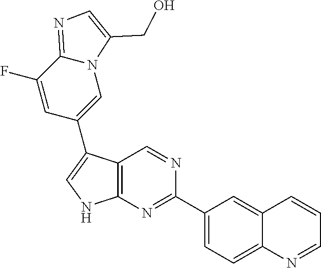 [SMILES: OCc1cnc2c(F)cc(-c3c[nH]c4nc(-c5ccc6ncccc6c5)ncc34)cn12] | 400 |
| 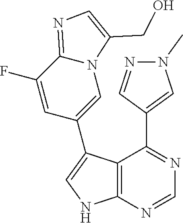 [SMILES: Cn1cc(-c2ncnc3[nH]cc(-c4cc(F)c5ncc(CO)n5c4)c23)cn1] | 401 |
| 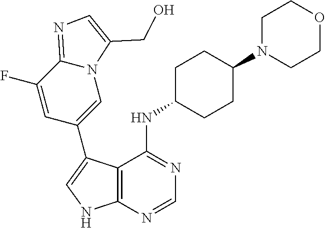 [SMILES: OCc1cnc2c(F)cc(-c3c[nH]c4ncnc(N[C@H]5CC[C@H](N6CCOCC6)CC5)c34)cn12] | 402 |
| 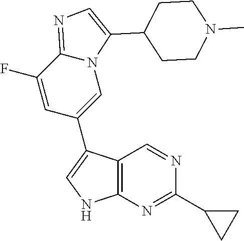 [SMILES: CN1CCC(c2cnc3c(F)cc(-c4c[nH]c5nc(C6CC6)ncc45)cn23)CC1] | 403 |
| 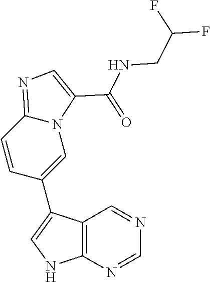 [SMILES: O=C(NCC(F)F)c1cnc2ccc(-c3c[nH]c4ncncc34)cn12] | 404 |
| 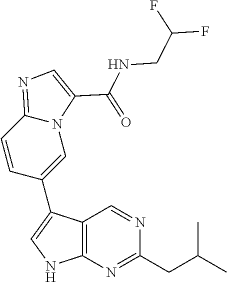 [SMILES: CC(C)Cc1ncc2c(-c3ccc4ncc(C(=O)NCC(F)F)n4c3)c[nH]c2n1] | 405 |
| 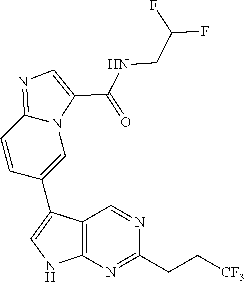 [SMILES: O=C(NCC(F)F)c1cnc2ccc(-c3c[nH]c4nc(CCC(F)(F)F)ncc34)cn12] | 406 |
| 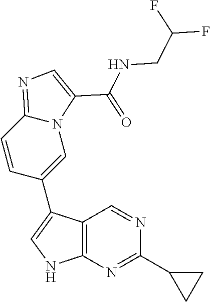 [SMILES: O=C(NCC(F)F)c1cnc2ccc(-c3c[nH]c4nc(C5CC5)ncc34)cn12] | 407 |
| 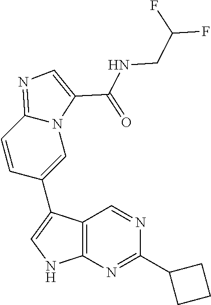 [SMILES: O=C(NCC(F)F)c1cnc2ccc(-c3c[nH]c4nc(C5CCC5)ncc34)cn12] | 408 |
| 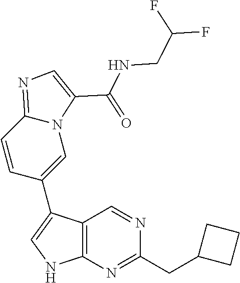 [SMILES: O=C(NCC(F)F)c1cnc2ccc(-c3c[nH]c4nc(CC5CCC5)ncc34)cn12] | 409 |
| 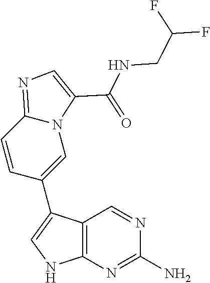 [SMILES: Nc1ncc2c(-c3ccc4ncc(C(=O)NCC(F)F)n4c3)c[nH]c2n1] | 410 |
| 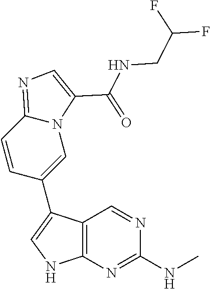 [SMILES: CNc1ncc2c(-c3ccc4ncc(C(=O)NCC(F)F)n4c3)c[nH]c2n1] | 411 |
| 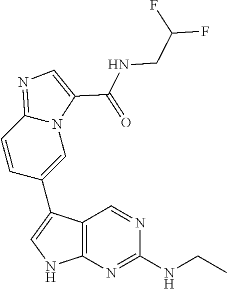 [SMILES: CCNc1ncc2c(-c3ccc4ncc(C(=O)NCC(F)F)n4c3)c[nH]c2n1] | 412 |
| 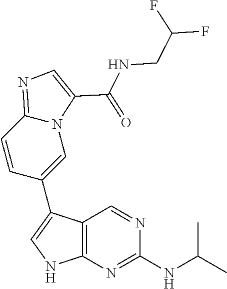 [SMILES: CC(C)Nc1ncc2c(-c3ccc4ncc(C(=O)NCC(F)F)n4c3)c[nH]c2n1] | 413 |
| 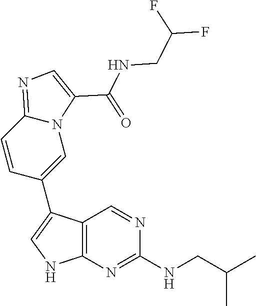 [SMILES: CC(C)CNc1ncc2c(-c3ccc4ncc(C(=O)NCC(F)F)n4c3)c[nH]c2n1] | 414 |
| 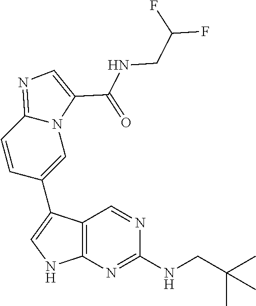 [SMILES: CC(C)(C)CNc1ncc2c(-c3ccc4ncc(C(=O)NCC(F)F)n4c3)c[nH]c2n1] | 415 |
| 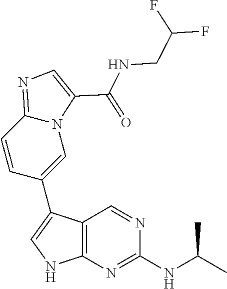 | 416 |
| 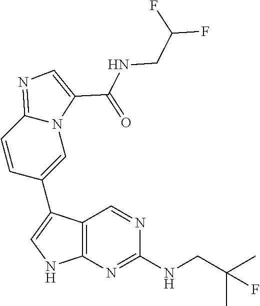 [SMILES: CC(C)(F)CNc1ncc2c(-c3ccc4ncc(C(=O)NCC(F)F)n4c3)c[nH]c2n1] | 417 |
| 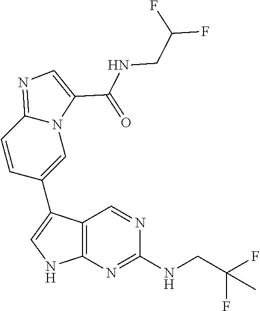 [SMILES: CC(F)(F)CNc1ncc2c(-c3ccc4ncc(C(=O)NCC(F)F)n4c3)c[nH]c2n1] | 418 |
| 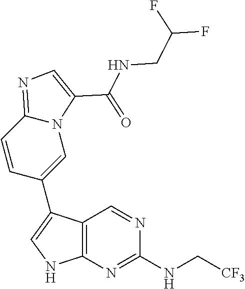 [SMILES: O=C(NCC(F)F)c1cnc2ccc(-c3c[nH]c4nc(NCC(F)(F)F)ncc34)cn12] | 419 |
| 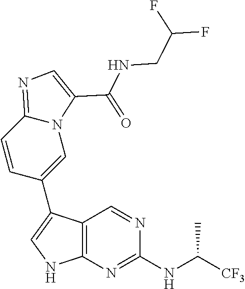 [SMILES: C[C@@H](Nc1ncc2c(-c3ccc4ncc(C(=O)NCC(F)F)n4c3)c[nH]c2n1)C(F)(F)F] | 420 |
| 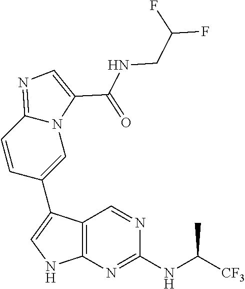 [SMILES: C[C@H](Nc1ncc2c(-c3ccc4ncc(C(=O)NCC(F)F)n4c3)c[nH]c2n1)C(F)(F)F] | 421 |
| 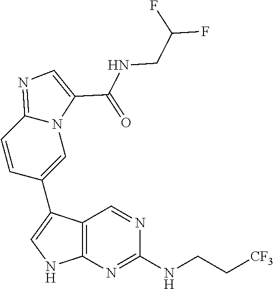 [SMILES: O=C(NCC(F)F)c1cnc2ccc(-c3c[nH]c4nc(NCCC(F)(F)F)ncc34)cn12] | 422 |
| 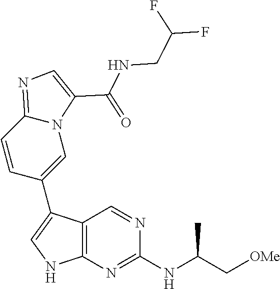 [SMILES: COC[C@H](C)Nc1ncc2c(-c3ccc4ncc(C(=O)NCC(F)F)n4c3)c[nH]c2n1] | 423 |
| 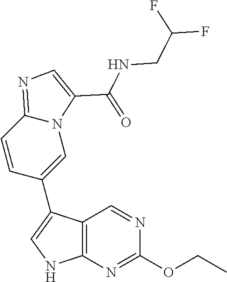 [SMILES: CCOc1ncc2c(-c3ccc4ncc(C(=O)NCC(F)F)n4c3)c[nH]c2n1] | 424 |
| 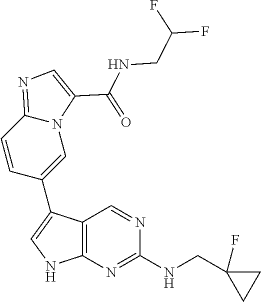 [SMILES: O=C(NCC(F)F)c1cnc2ccc(-c3c[nH]c4nc(NCC5(F)CC5)ncc34)cn12] | 425 |
| 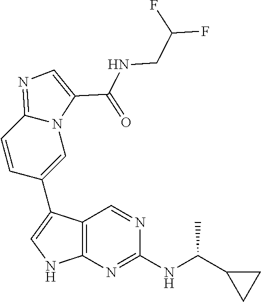 [SMILES: C[C@@H](Nc1ncc2c(-c3ccc4ncc(C(=O)NCC(F)F)n4c3)c[nH]c2n1)C1CC1] | 426 |
| 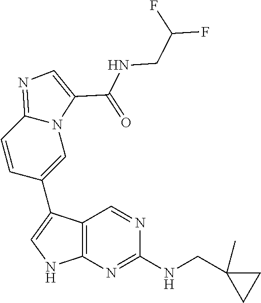 [SMILES: CC1(CNc2ncc3c(-c4ccc5ncc(C(=O)NCC(F)F)n5c4)c[nH]c3n2)CC1] | 427 |
| 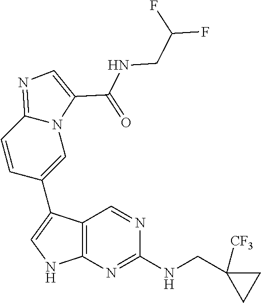 [SMILES: O=C(NCC(F)F)c1cnc2ccc(-c3c[nH]c4nc(NCC5(C(F)(F)F)CC5)ncc34)cn12] | 428 |
| 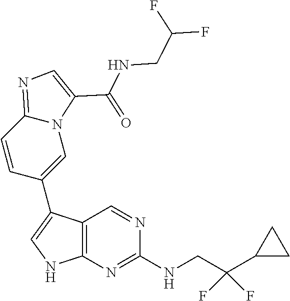 [SMILES: O=C(NCC(F)F)c1cnc2ccc(-c3c[nH]c4nc(NCC(F)(F)C5CC5)ncc34)cn12] | 429 |
| 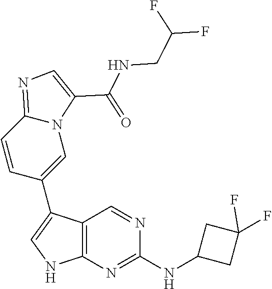 [SMILES: O=C(NCC(F)F)c1cnc2ccc(-c3c[nH]c4nc(NC5CC(F)(F)C5)ncc34)cn12] | 430 |
| 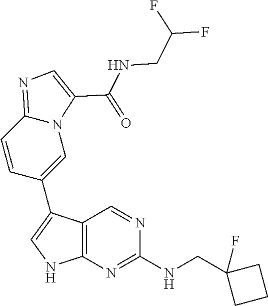 [SMILES: O=C(NCC(F)F)c1cnc2ccc(-c3c[nH]c4nc(NCC5(F)CCC5)ncc34)cn12] | 431 |
| 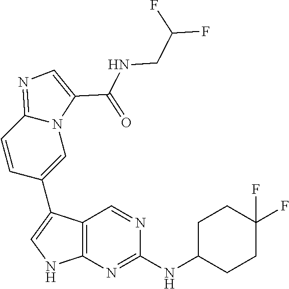 [SMILES: O=C(NCC(F)F)c1cnc2ccc(-c3c[nH]c4nc(NC5CCC(F)(F)CC5)ncc34)cn12] | 432 |
| 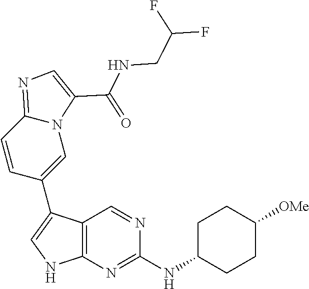 [SMILES: CO[C@H]1CC[C@@H](Nc2ncc3c(-c4ccc5ncc(C(=O)NCC(F)F)n5c4)c[nH]c3n2)CC1] | 433 |
| 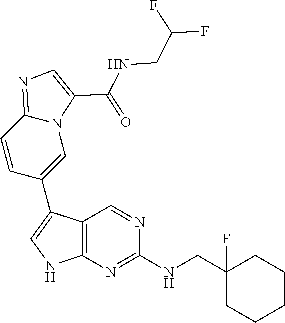 [SMILES: O=C(NCC(F)F)c1cnc2ccc(-c3c[nH]c4nc(NCC5(F)CCCCC5)ncc34)cn12] | 434 |
| 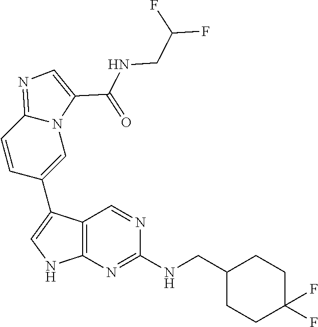 [SMILES: O=C(NCC(F)F)c1cnc2ccc(-c3c[nH]c4nc(NCC5CCC(F)(F)CC5)ncc34)cn12] | 435 |
| 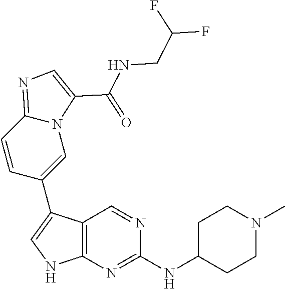 [SMILES: CN1CCC(Nc2ncc3c(-c4ccc5ncc(C(=O)NCC(F)F)n5c4)c[nH]c3n2)CC1] | 436 |
| 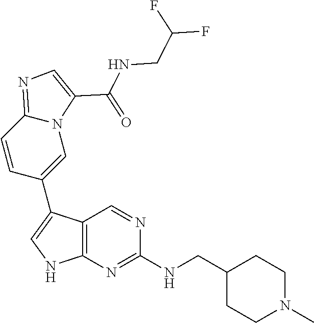 [SMILES: CN1CCC(CNc2ncc3c(-c4ccc5ncc(C(=O)NCC(F)F)n5c4)c[nH]c3n2)CC1] | 437 |
| 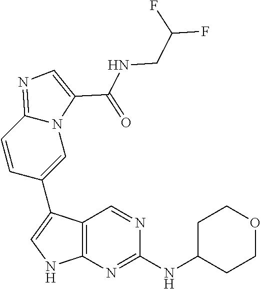 [SMILES: O=C(NCC(F)F)c1cnc2ccc(-c3c[nH]c4nc(NC5CCOCC5)ncc34)cn12] | 438 |
| 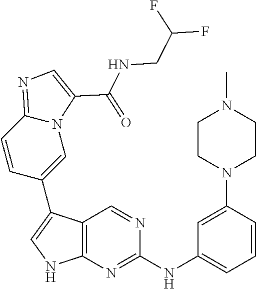 [SMILES: CN1CCN(c2cccc(Nc3ncc4c(-c5ccc6ncc(C(=O)NCC(F)F)n6c5)c[nH]c4n3)c2)CC1] | 439 |
| 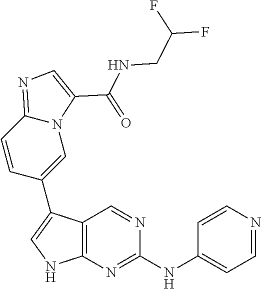 [SMILES: O=C(NCC(F)F)c1cnc2ccc(-c3c[nH]c4nc(Nc5ccncc5)ncc34)cn12] | 440 |
| 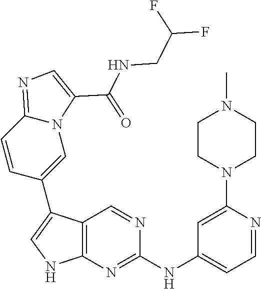 [SMILES: CN1CCN(c2cc(Nc3ncc4c(-c5ccc6ncc(C(=O)NCC(F)F)n6c5)c[nH]c4n3)ccn2)CC1] | 441 |
| 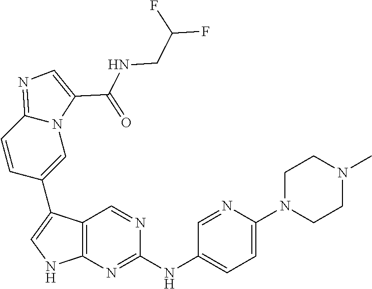 [SMILES: CN1CCN(c2ccc(Nc3ncc4c(-c5ccc6ncc(C(=O)NCC(F)F)n6c5)c[nH]c4n3)cn2)CC1] | 442 |
| 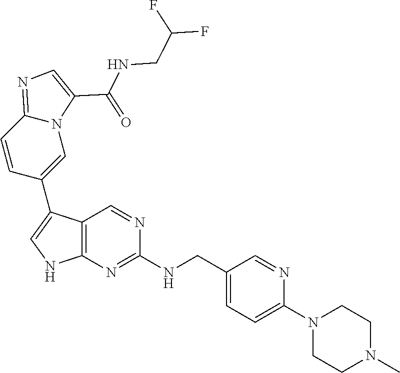 [SMILES: CN1CCN(c2ccc(CNc3ncc4c(-c5ccc6ncc(C(=O)NCC(F)F)n6c5)c[nH]c4n3)cn2)CC1] | 443 |
| 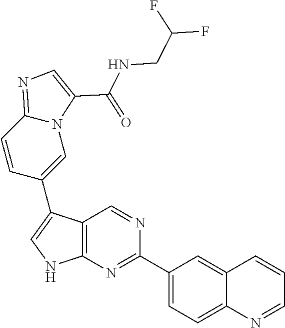 [SMILES: O=C(NCC(F)F)c1cnc2ccc(-c3c[nH]c4nc(-c5ccc6ncccc6c5)ncc34)cn12] | 444 |
| 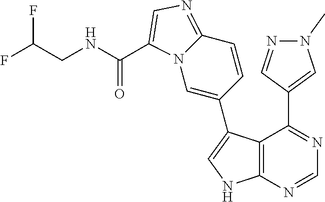 [SMILES: Cn1cc(-c2ncnc3[nH]cc(-c4ccc5ncc(C(=O)NCC(F)F)n5c4)c23)cn1] | 445 |
| 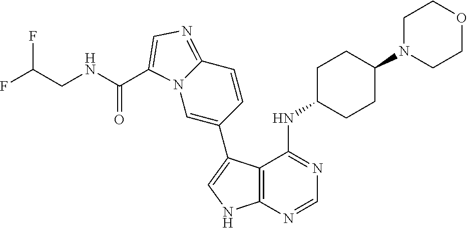 [SMILES: O=C(NCC(F)F)c1cnc2ccc(-c3c[nH]c4ncnc(N[C@H]5CC[C@H](N6CCOCC6)CC5)c34)cn12] | 446 |
| 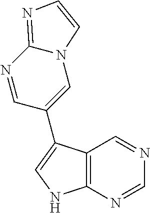 [SMILES: c1ncc2c(-c3cnc4nccn4c3)c[nH]c2n1] | 447 |
| 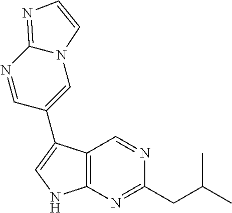 [SMILES: CC(C)Cc1ncc2c(-c3cnc4nccn4c3)c[nH]c2n1] | 448 |
| 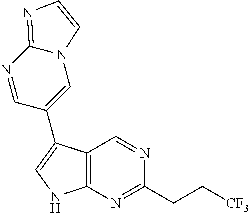 [SMILES: FC(F)(F)CCc1ncc2c(-c3cnc4nccn4c3)c[nH]c2n1] | 449 |
| 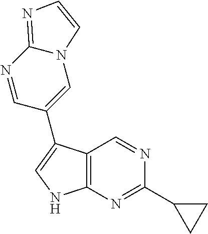 [SMILES: c1cn2cc(-c3c[nH]c4nc(C5CC5)ncc34)cnc2n1] | 450 |
| 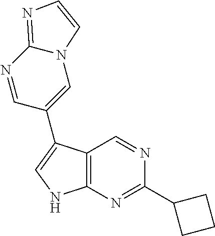 [SMILES: c1cn2cc(-c3c[nH]c4nc(C5CCC5)ncc34)cnc2n1] | 451 |
| 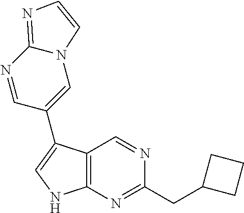 [SMILES: c1cn2cc(-c3c[nH]c4nc(CC5CCC5)ncc34)cnc2n1] | 452 |
| 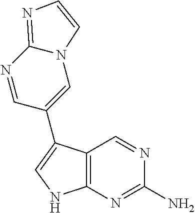 [SMILES: Nc1ncc2c(-c3cnc4nccn4c3)c[nH]c2n1] | 453 |
| 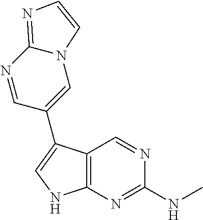 [SMILES: CNc1ncc2c(-c3cnc4nccn4c3)c[nH]c2n1] | 454 |
| 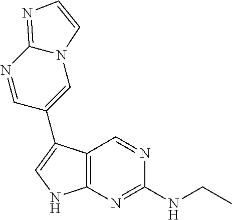 [SMILES: CCNc1ncc2c(-c3cnc4nccn4c3)c[nH]c2n1] | 455 |
| 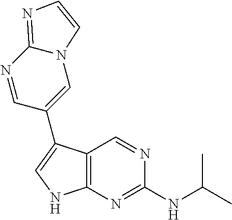 [SMILES: CC(C)Nc1ncc2c(-c3cnc4nccn4c3)c[nH]c2n1] | 456 |
| 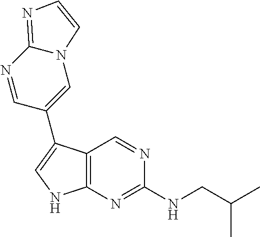 [SMILES: CC(C)CNc1ncc2c(-c3cnc4nccn4c3)c[nH]c2n1] | 457 |
| 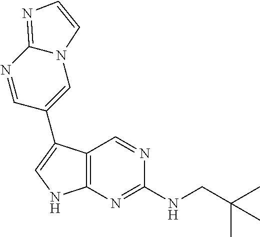 [SMILES: CC(C)(C)CNc1ncc2c(-c3cnc4nccn4c3)c[nH]c2n1] | 458 |
| 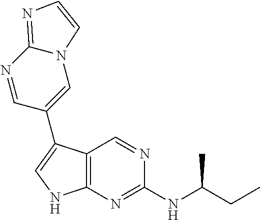 [SMILES: CC[C@H](C)Nc1ncc2c(-c3cnc4nccn4c3)c[nH]c2n1] | 459 |
| 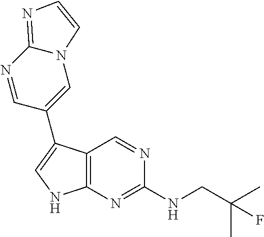 [SMILES: CC(C)(F)CNc1ncc2c(-c3cnc4nccn4c3)c[nH]c2n1] | 460 |
| 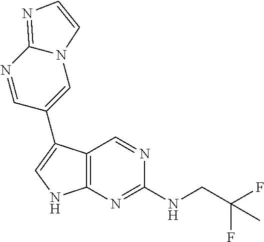 [SMILES: CC(F)(F)CNc1ncc2c(-c3cnc4nccn4c3)c[nH]c2n1] | 461 |
| 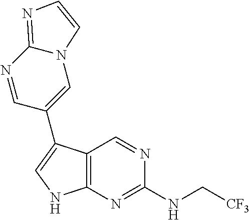 [SMILES: FC(F)(F)CNc1ncc2c(-c3cnc4nccn4c3)c[nH]c2n1] | 462 |
| 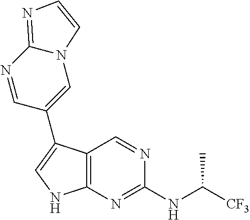 [SMILES: C[C@@H](Nc1ncc2c(-c3cnc4nccn4c3)c[nH]c2n1)C(F)(F)F] | 463 |
| 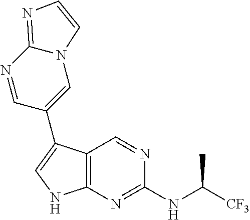 [SMILES: C[C@H](Nc1ncc2c(-c3cnc4nccn4c3)c[nH]c2n1)C(F)(F)F] | 464 |
| 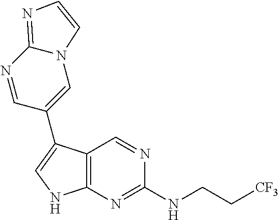 [SMILES: FC(F)(F)CCNc1ncc2c(-c3cnc4nccn4c3)c[nH]c2n1] | 465 |
| 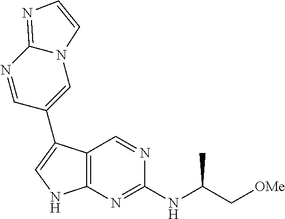 [SMILES: COC[C@H](C)Nc1ncc2c(-c3cnc4nccn4c3)c[nH]c2n1] | 466 |
| 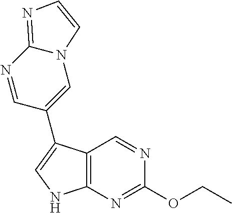 [SMILES: CCOc1ncc2c(-c3cnc4nccn4c3)c[nH]c2n1] | 467 |
| 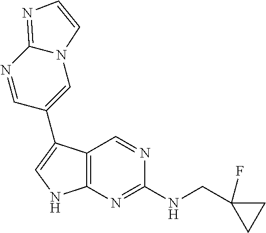 [SMILES: FC1(CNc2ncc3c(-c4cnc5nccn5c4)c[nH]c3n2)CC1] | 468 |
| 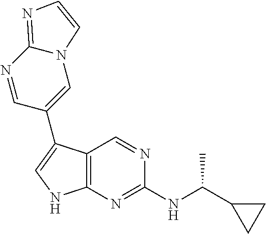 [SMILES: C[C@@H](Nc1ncc2c(-c3cnc4nccn4c3)c[nH]c2n1)C1CC1] | 469 |
| 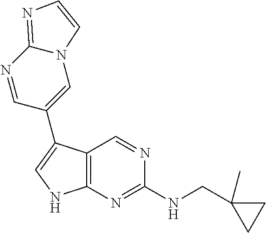 [SMILES: CC1(CNc2ncc3c(-c4cnc5nccn5c4)c[nH]c3n2)CC1] | 470 |
| 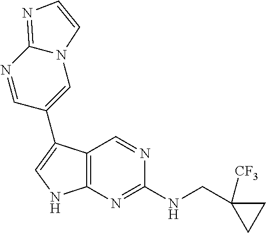 [SMILES: FC(F)(F)C1(CNc2ncc3c(-c4cnc5nccn5c4)c[nH]c3n2)CC1] | 471 |
| 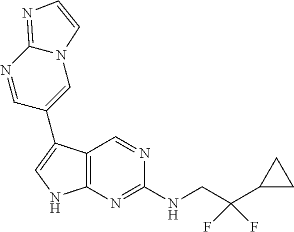 [SMILES: FC(F)(CNc1ncc2c(-c3cnc4nccn4c3)c[nH]c2n1)C1CC1] | 472 |
| 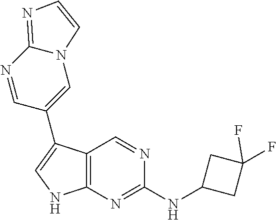 [SMILES: FC1(F)CC(Nc2ncc3c(-c4cnc5nccn5c4)c[nH]c3n2)C1] | 473 |
| 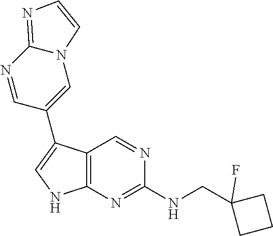 [SMILES: FC1(CNc2ncc3c(-c4cnc5nccn5c4)c[nH]c3n2)CCC1] | 474 |
| 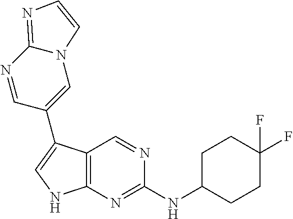 [SMILES: FC1(F)CCC(Nc2ncc3c(-c4cnc5nccn5c4)c[nH]c3n2)CC1] | 475 |
| 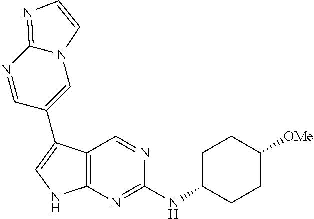 [SMILES: CO[C@H]1CC[C@@H](Nc2ncc3c(-c4cnc5nccn5c4)c[nH]c3n2)CC1] | 476 |
| 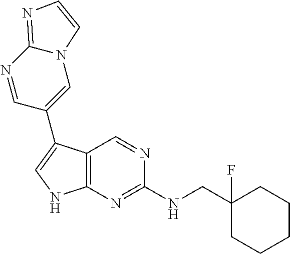 [SMILES: FC1(CNc2ncc3c(-c4cnc5nccn5c4)c[nH]c3n2)CCCCC1] | 477 |
| 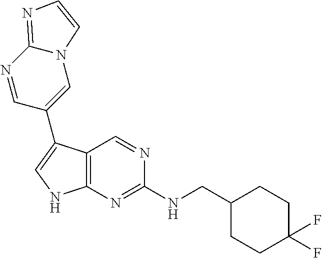 [SMILES: FC1(F)CCC(CNc2ncc3c(-c4cnc5nccn5c4)c[nH]c3n2)CC1] | 478 |
| 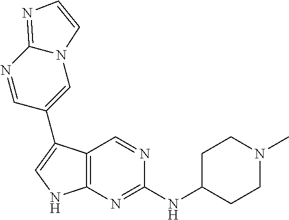 [SMILES: CN1CCC(Nc2ncc3c(-c4cnc5nccn5c4)c[nH]c3n2)CC1] | 479 |
| 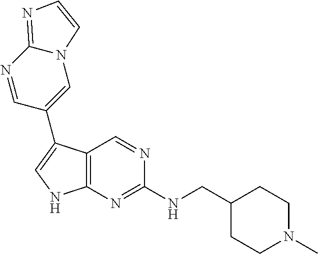 [SMILES: CN1CCC(CNc2ncc3c(-c4cnc5nccn5c4)c[nH]c3n2)CC1] | 480 |
| 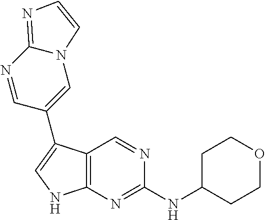 [SMILES: c1cn2cc(-c3c[nH]c4nc(NC5CCOCC5)ncc34)cnc2n1] | 481 |
| 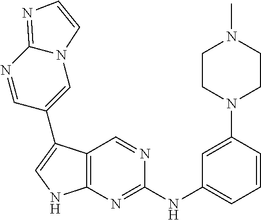 [SMILES: CN1CCN(c2cccc(Nc3ncc4c(-c5cnc6nccn6c5)c[nH]c4n3)c2)CC1] | 482 |
| 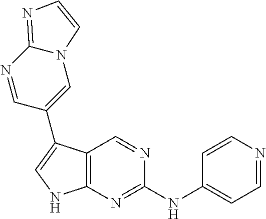 [SMILES: c1cc(Nc2ncc3c(-c4cnc5nccn5c4)c[nH]c3n2)ccn1] | 483 |
| 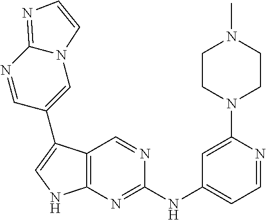 [SMILES: CN1CCN(c2cc(Nc3ncc4c(-c5cnc6nccn6c5)c[nH]c4n3)ccn2)CC1] | 484 |
| 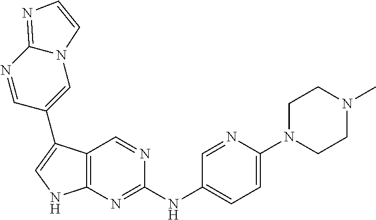 [SMILES: CN1CCN(c2ccc(Nc3ncc4c(-c5cnc6nccn6c5)c[nH]c4n3)cn2)CC1] | 485 |
| 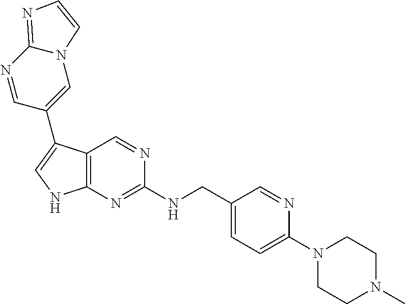 [SMILES: CN1CCN(c2ccc(CNc3ncc4c(-c5cnc6nccn6c5)c[nH]c4n3)cn2)CC1] | 486 |
| 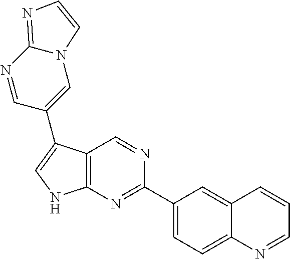 [SMILES: c1cnc2ccc(-c3ncc4c(-c5cnc6nccn6c5)c[nH]c4n3)cc2c1] | 487 |
| 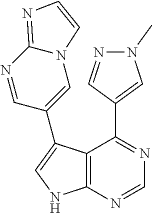 [SMILES: Cn1cc(-c2ncnc3[nH]cc(-c4cnc5nccn5c4)c23)cn1] | 488 |
| 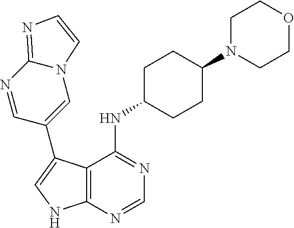 [SMILES: c1nc(N[C@H]2CC[C@H](N3CCOCC3)CC2)c2c(-c3cnc4nccn4c3)c[nH]c2n1] | 489 |
| 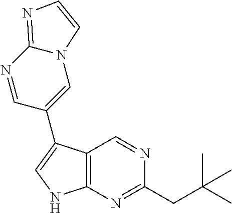 [SMILES: CC(C)(C)Cc1ncc2c(-c3cnc4nccn4c3)c[nH]c2n1] | 490 |
| 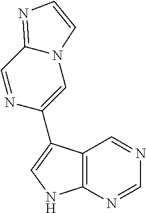 [SMILES: c1ncc2c(-c3cn4ccnc4cn3)c[nH]c2n1] | 491 |
| 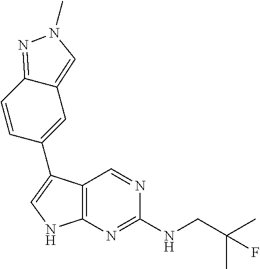 [SMILES: Cn1cc2cc(-c3c[nH]c4nc(NCC(C)(C)F)ncc34)ccc2n1] | 492 |
| 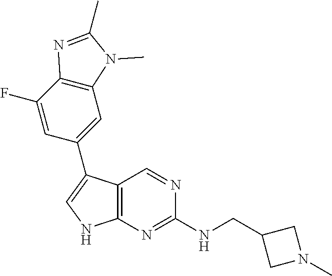 [SMILES: Cc1nc2c(F)cc(-c3c[nH]c4nc(NCC5CN(C)C5)ncc34)cc2n1C] | 493 |
| 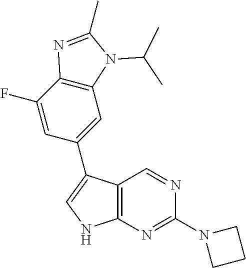 [SMILES: Cc1nc2c(F)cc(-c3c[nH]c4nc(N5CCC5)ncc34)cc2n1C(C)C] | 494 |
| 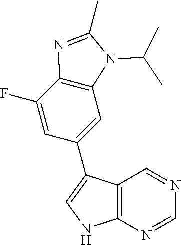 [SMILES: Cc1nc2c(F)cc(-c3c[nH]c4ncncc34)cc2n1C(C)C] | 495 |
| 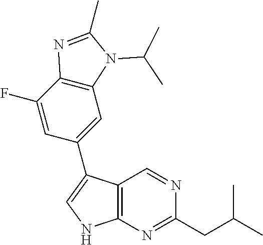 [SMILES: Cc1nc2c(F)cc(-c3c[nH]c4nc(CC(C)C)ncc34)cc2n1C(C)C] | 496 |
| 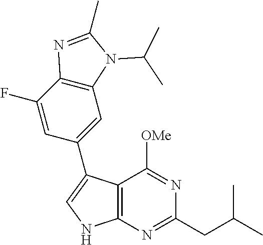 [SMILES: COc1nc(CC(C)C)nc2[nH]cc(-c3cc(F)c4nc(C)n(C(C)C)c4c3)c12] | 497 |
| 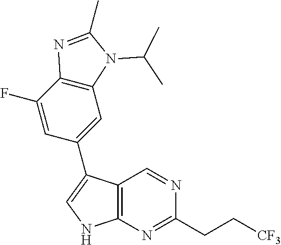 [SMILES: Cc1nc2c(F)cc(-c3c[nH]c4nc(CCC(F)(F)F)ncc34)cc2n1C(C)C] | 498 |
| 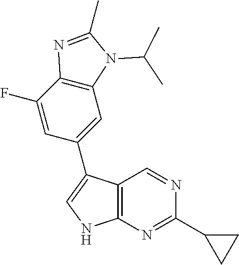 [SMILES: Cc1nc2c(F)cc(-c3c[nH]c4nc(C5CC5)ncc34)cc2n1C(C)C] | 499 |
| 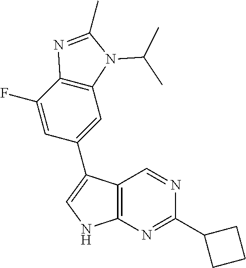 [SMILES: Cc1nc2c(F)cc(-c3c[nH]c4nc(C5CCC5)ncc34)cc2n1C(C)C] | 500 |
| 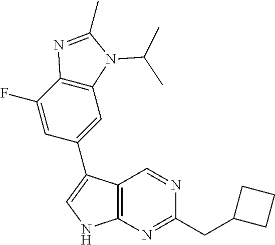 [SMILES: Cc1nc2c(F)cc(-c3c[nH]c4nc(CC5CCC5)ncc34)cc2n1C(C)C] | 501 |
| 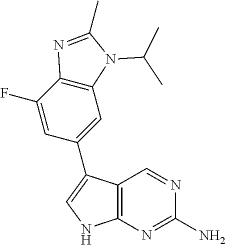 [SMILES: Cc1nc2c(F)cc(-c3c[nH]c4nc(N)ncc34)cc2n1C(C)C] | 502 |
| 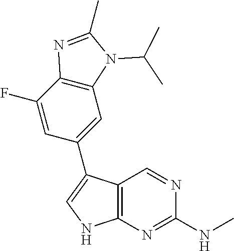 [SMILES: CNc1ncc2c(-c3cc(F)c4nc(C)n(C(C)C)c4c3)c[nH]c2n1] | 503 |
| 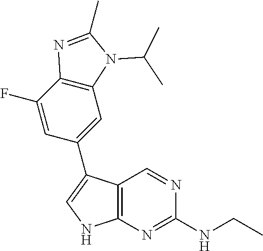 [SMILES: CCNc1ncc2c(-c3cc(F)c4nc(C)n(C(C)C)c4c3)c[nH]c2n1] | 504 |
| 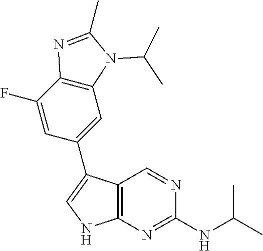 [SMILES: Cc1nc2c(F)cc(-c3c[nH]c4nc(NC(C)C)ncc34)cc2n1C(C)C] | 505 |
| 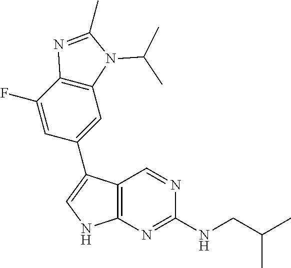 [SMILES: Cc1nc2c(F)cc(-c3c[nH]c4nc(NCC(C)C)ncc34)cc2n1C(C)C] | 506 |
| 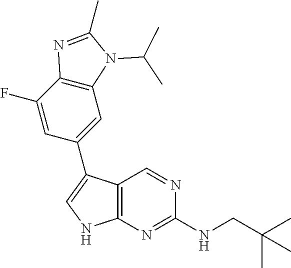 [SMILES: Cc1nc2c(F)cc(-c3c[nH]c4nc(NCC(C)(C)C)ncc34)cc2n1C(C)C] | 507 |
| 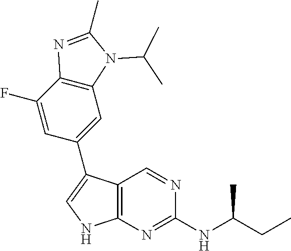 [SMILES: CC[C@H](C)Nc1ncc2c(-c3cc(F)c4nc(C)n(C(C)C)c4c3)c[nH]c2n1] | 508 |
| 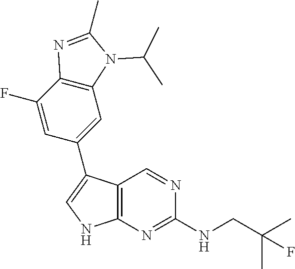 [SMILES: Cc1nc2c(F)cc(-c3c[nH]c4nc(NCC(C)(C)F)ncc34)cc2n1C(C)C] | 509 |
| 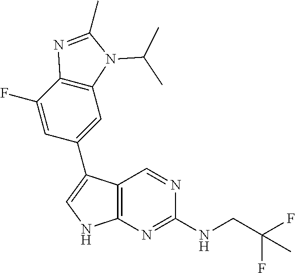 [SMILES: Cc1nc2c(F)cc(-c3c[nH]c4nc(NCC(C)(F)F)ncc34)cc2n1C(C)C] | 510 |
| 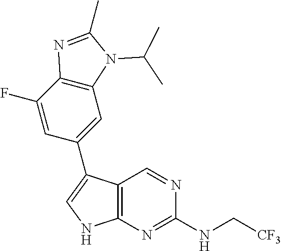 [SMILES: Cc1nc2c(F)cc(-c3c[nH]c4nc(NCC(F)(F)F)ncc34)cc2n1C(C)C] | 511 |
| 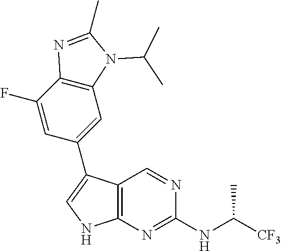 [SMILES: Cc1nc2c(F)cc(-c3c[nH]c4nc(N[C@H](C)C(F)(F)F)ncc34)cc2n1C(C)C] | 512 |
| 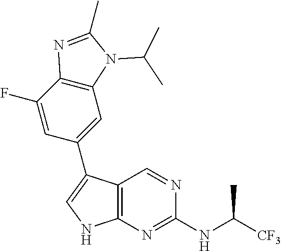 [SMILES: Cc1nc2c(F)cc(-c3c[nH]c4nc(N[C@@H](C)C(F)(F)F)ncc34)cc2n1C(C)C] | 513 |
| 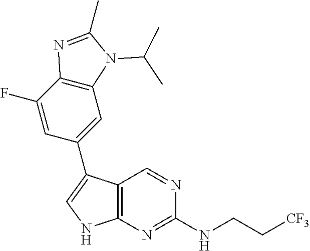 [SMILES: Cc1nc2c(F)cc(-c3c[nH]c4nc(NCCC(F)(F)F)ncc34)cc2n1C(C)C] | 514 |
| 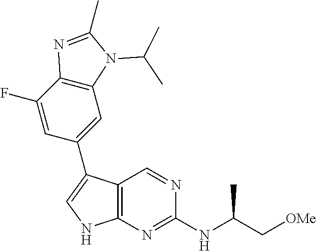 [SMILES: COC[C@H](C)Nc1ncc2c(-c3cc(F)c4nc(C)n(C(C)C)c4c3)c[nH]c2n1] | 515 |
| 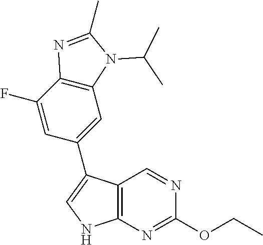 [SMILES: CCOc1ncc2c(-c3cc(F)c4nc(C)n(C(C)C)c4c3)c[nH]c2n1] | 516 |
| 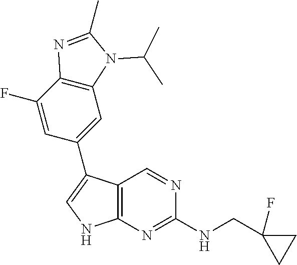 [SMILES: Cc1nc2c(F)cc(-c3c[nH]c4nc(NCC5(F)CC5)ncc34)cc2n1C(C)C] | 517 |
| 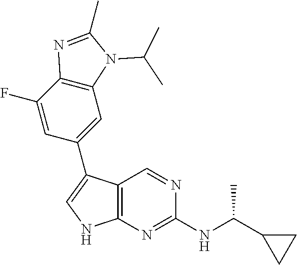 [SMILES: Cc1nc2c(F)cc(-c3c[nH]c4nc(N[C@H](C)C5CC5)ncc34)cc2n1C(C)C] | 518 |
| 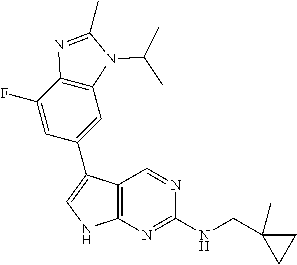 [SMILES: Cc1nc2c(F)cc(-c3c[nH]c4nc(NCC5(C)CC5)ncc34)cc2n1C(C)C] | 519 |
| 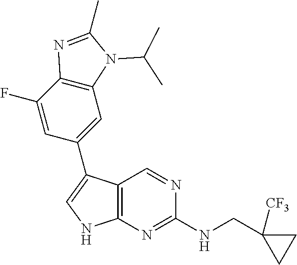 [SMILES: Cc1nc2c(F)cc(-c3c[nH]c4nc(NCC5(C(F)(F)F)CC5)ncc34)cc2n1C(C)C] | 520 |
| 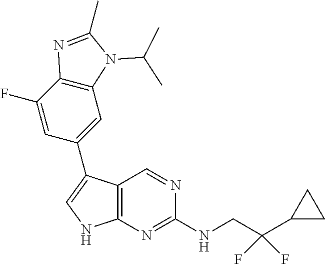 [SMILES: Cc1nc2c(F)cc(-c3c[nH]c4nc(NCC(F)(F)C5CC5)ncc34)cc2n1C(C)C] | 521 |
| 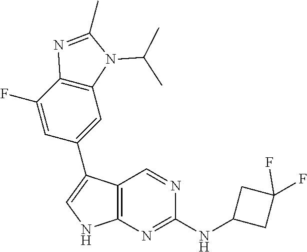 [SMILES: Cc1nc2c(F)cc(-c3c[nH]c4nc(NC5CC(F)(F)C5)ncc34)cc2n1C(C)C] | 522 |
| 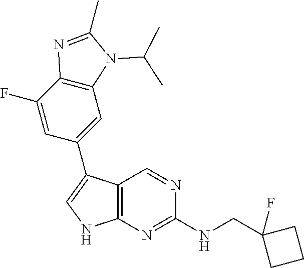 [SMILES: Cc1nc2c(F)cc(-c3c[nH]c4nc(NCC5(F)CCC5)ncc34)cc2n1C(C)C] | 523 |
| 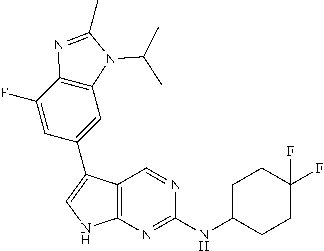 [SMILES: Cc1nc2c(F)cc(-c3c[nH]c4nc(NC5CCC(F)(F)CC5)ncc34)cc2n1C(C)C] | 524 |
| 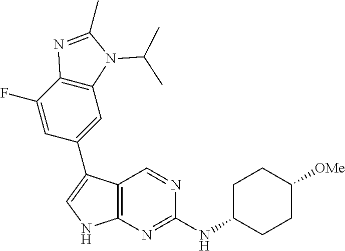 [SMILES: CO[C@H]1CC[C@@H](Nc2ncc3c(-c4cc(F)c5nc(C)n(C(C)C)c5c4)c[nH]c3n2)CC1] | 525 |
| 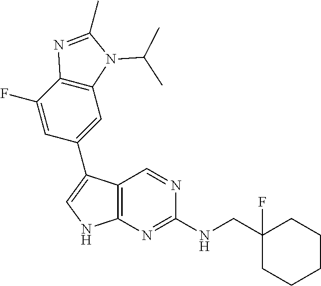 [SMILES: Cc1nc2c(F)cc(-c3c[nH]c4nc(NCC5(F)CCCCC5)ncc34)cc2n1C(C)C] | 526 |
| 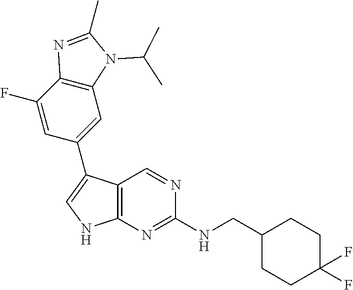 [SMILES: Cc1nc2c(F)cc(-c3c[nH]c4nc(NCC5CCC(F)(F)CC5)ncc34)cc2n1C(C)C] | 527 |
| 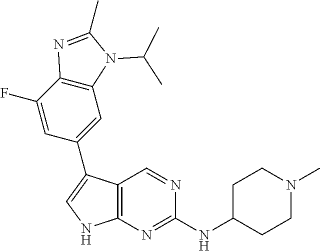 [SMILES: Cc1nc2c(F)cc(-c3c[nH]c4nc(NC5CCN(C)CC5)ncc34)cc2n1C(C)C] | 528 |
| 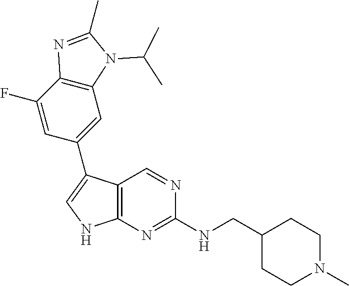 [SMILES: Cc1nc2c(F)cc(-c3c[nH]c4nc(NCC5CCN(C)CC5)ncc34)cc2n1C(C)C] | 529 |
| 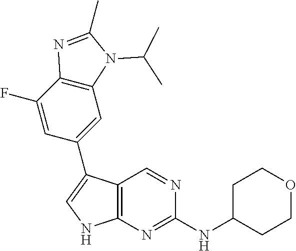 [SMILES: Cc1nc2c(F)cc(-c3c[nH]c4nc(NC5CCOCC5)ncc34)cc2n1C(C)C] | 530 |
| 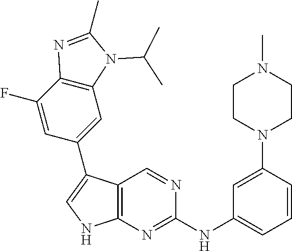 [SMILES: Cc1nc2c(F)cc(-c3c[nH]c4nc(Nc5cccc(N6CCN(C)CC6)c5)ncc34)cc2n1C(C)C] | 531 |
| 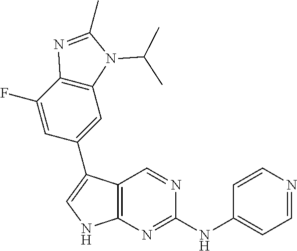 [SMILES: Cc1nc2c(F)cc(-c3c[nH]c4nc(Nc5ccncc5)ncc34)cc2n1C(C)C] | 532 |
| 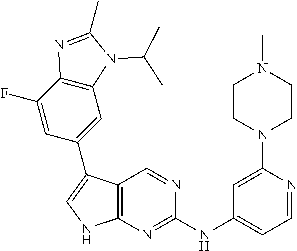 [SMILES: Cc1nc2c(F)cc(-c3c[nH]c4nc(Nc5ccnc(N6CCN(C)CC6)c5)ncc34)cc2n1C(C)C] | 533 |
| 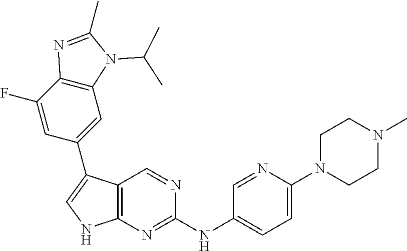 [SMILES: Cc1nc2c(F)cc(-c3c[nH]c4nc(Nc5ccc(N6CCN(C)CC6)nc5)ncc34)cc2n1C(C)C] | 534 |
| 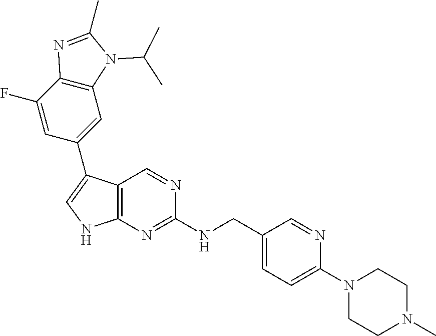 [SMILES: Cc1nc2c(F)cc(-c3c[nH]c4nc(NCc5ccc(N6CCN(C)CC6)nc5)ncc34)cc2n1C(C)C] | 535 |
| 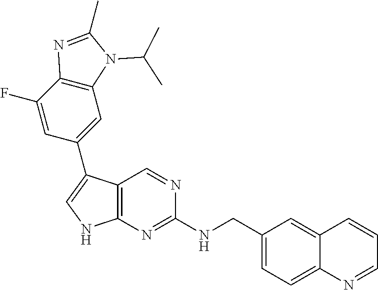 [SMILES: Cc1nc2c(F)cc(-c3c[nH]c4nc(NCc5ccc6ncccc6c5)ncc34)cc2n1C(C)C] | 536 |
| 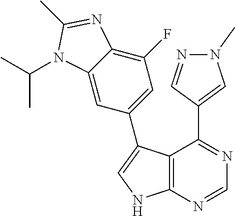 [SMILES: Cc1nc2c(F)cc(-c3c[nH]c4ncnc(-c5cnn(C)c5)c34)cc2n1C(C)C] | 537 |
| 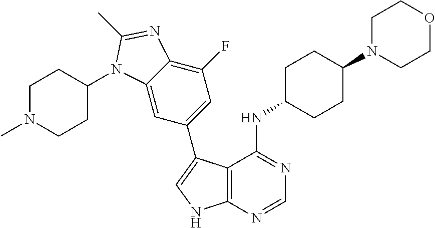 [SMILES: Cc1nc2c(F)cc(-c3c[nH]c4ncnc(N[C@H]5CC[C@H](N6CCOCC6)CC5)c34)cc2n1C1CCN(C)CC1] | 538 |
| 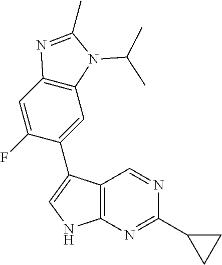 [SMILES: Cc1nc2cc(F)c(-c3c[nH]c4nc(C5CC5)ncc34)cc2n1C(C)C] | 539 |
| 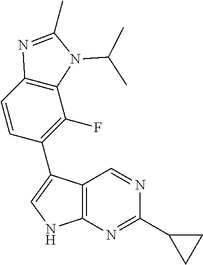 [SMILES: Cc1nc2ccc(-c3c[nH]c4nc(C5CC5)ncc34)c(F)c2n1C(C)C] | 540 |
| 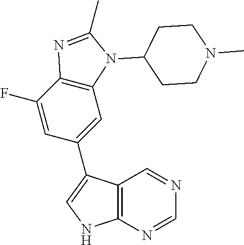 [SMILES: Cc1nc2c(F)cc(-c3c[nH]c4ncncc34)cc2n1C1CCN(C)CC1] | 541 |
| 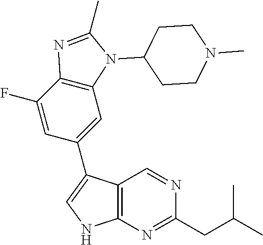 [SMILES: Cc1nc2c(F)cc(-c3c[nH]c4nc(CC(C)C)ncc34)cc2n1C1CCN(C)CC1] | 542 |
| 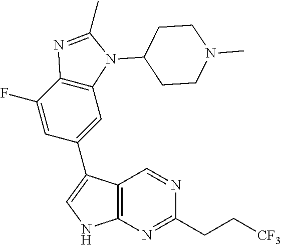 [SMILES: Cc1nc2c(F)cc(-c3c[nH]c4nc(CCC(F)(F)F)ncc34)cc2n1C1CCN(C)CC1] | 543 |
| 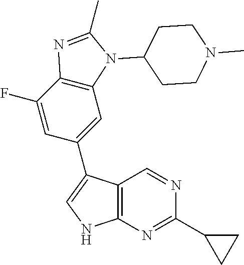 [SMILES: Cc1nc2c(F)cc(-c3c[nH]c4nc(C5CC5)ncc34)cc2n1C1CCN(C)CC1] | 544 |
| 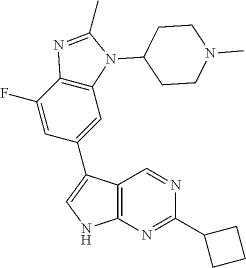 [SMILES: Cc1nc2c(F)cc(-c3c[nH]c4nc(C5CCC5)ncc34)cc2n1C1CCN(C)CC1] | 545 |
| 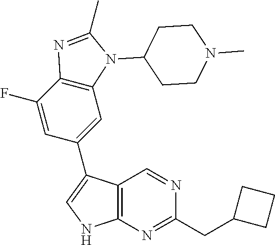 [SMILES: Cc1nc2c(F)cc(-c3c[nH]c4nc(CC5CCC5)ncc34)cc2n1C1CCN(C)CC1] | 546 |
| 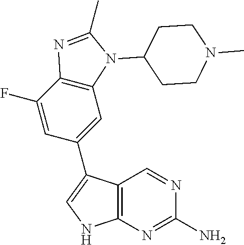 [SMILES: Cc1nc2c(F)cc(-c3c[nH]c4nc(N)ncc34)cc2n1C1CCN(C)CC1] | 547 |
| 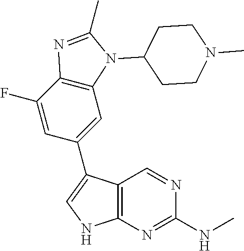 [SMILES: CNc1ncc2c(-c3cc(F)c4nc(C)n(C5CCN(C)CC5)c4c3)c[nH]c2n1] | 548 |
| 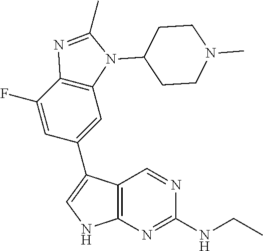 [SMILES: CCNc1ncc2c(-c3cc(F)c4nc(C)n(C5CCN(C)CC5)c4c3)c[nH]c2n1] | 549 |
| 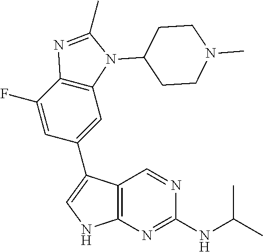 [SMILES: Cc1nc2c(F)cc(-c3c[nH]c4nc(NC(C)C)ncc34)cc2n1C1CCN(C)CC1] | 550 |
| 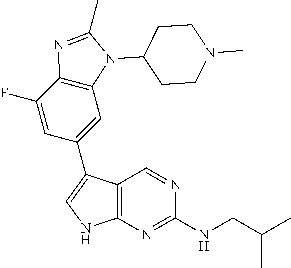 [SMILES: Cc1nc2c(F)cc(-c3c[nH]c4nc(NCC(C)C)ncc34)cc2n1C1CCN(C)CC1] | 551 |
| 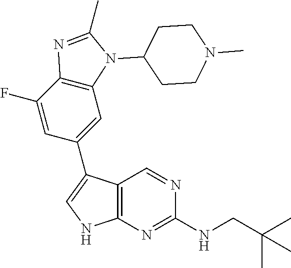 [SMILES: Cc1nc2c(F)cc(-c3c[nH]c4nc(NCC(C)(C)C)ncc34)cc2n1C1CCN(C)CC1] | 552 |
| 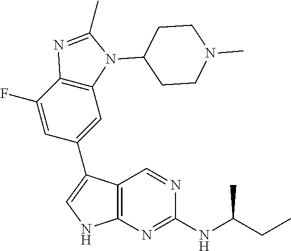 [SMILES: CC[C@H](C)Nc1ncc2c(-c3cc(F)c4nc(C)n(C5CCN(C)CC5)c4c3)c[nH]c2n1] | 553 |
| 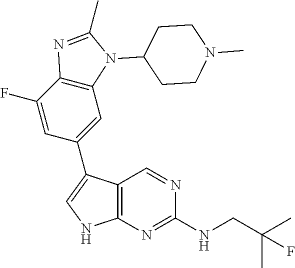 [SMILES: Cc1nc2c(F)cc(-c3c[nH]c4nc(NCC(C)(C)F)ncc34)cc2n1C1CCN(C)CC1] | 554 |
| 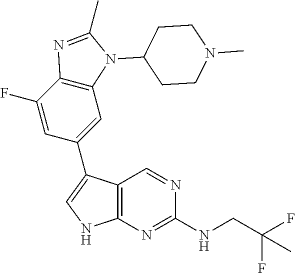 [SMILES: Cc1nc2c(F)cc(-c3c[nH]c4nc(NCC(C)(F)F)ncc34)cc2n1C1CCN(C)CC1] | 555 |
| 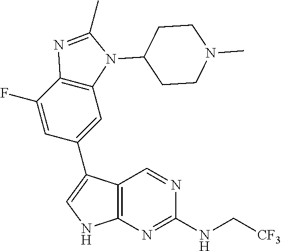 [SMILES: Cc1nc2c(F)cc(-c3c[nH]c4nc(NCC(F)(F)F)ncc34)cc2n1C1CCN(C)CC1] | 556 |
| 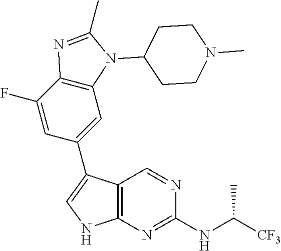 [SMILES: Cc1nc2c(F)cc(-c3c[nH]c4nc(N[C@H](C)C(F)(F)F)ncc34)cc2n1C1CCN(C)CC1] | 557 |
| 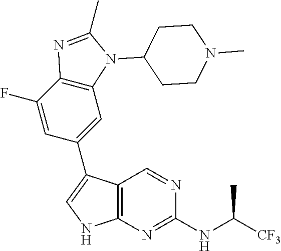 [SMILES: Cc1nc2c(F)cc(-c3c[nH]c4nc(N[C@@H](C)C(F)(F)F)ncc34)cc2n1C1CCN(C)CC1] | 558 |
| 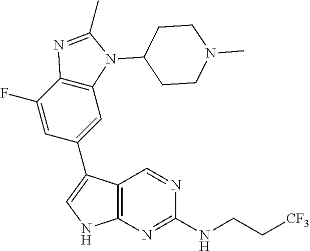 [SMILES: Cc1nc2c(F)cc(-c3c[nH]c4nc(NCCC(F)(F)F)ncc34)cc2n1C1CCN(C)CC1] | 559 |
| 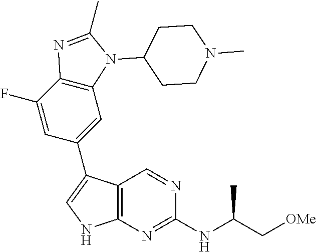 [SMILES: COC[C@H](C)Nc1ncc2c(-c3cc(F)c4nc(C)n(C5CCN(C)CC5)c4c3)c[nH]c2n1] | 560 |
| 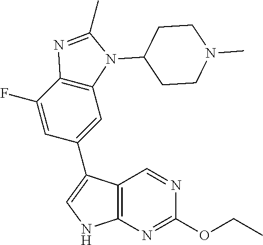 [SMILES: CCOc1ncc2c(-c3cc(F)c4nc(C)n(C5CCN(C)CC5)c4c3)c[nH]c2n1] | 561 |
| 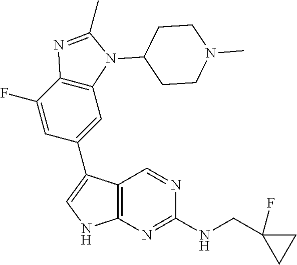 [SMILES: Cc1nc2c(F)cc(-c3c[nH]c4nc(NCC5(F)CC5)ncc34)cc2n1C1CCN(C)CC1] | 562 |
| 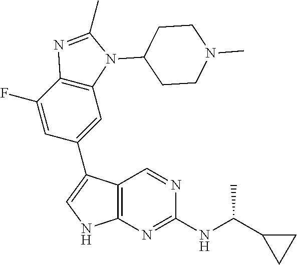 [SMILES: Cc1nc2c(F)cc(-c3c[nH]c4nc(N[C@H](C)C5CC5)ncc34)cc2n1C1CCN(C)CC1] | 563 |
| 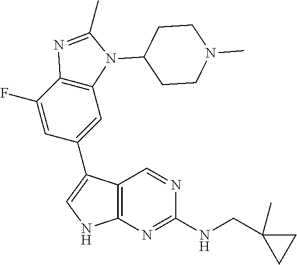 [SMILES: Cc1nc2c(F)cc(-c3c[nH]c4nc(NCC5(C)CC5)ncc34)cc2n1C1CCN(C)CC1] | 564 |
| 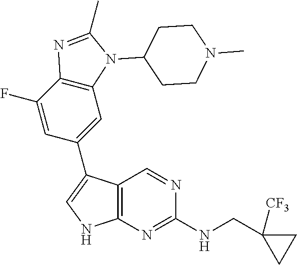 [SMILES: Cc1nc2c(F)cc(-c3c[nH]c4nc(NCC5(C(F)(F)F)CC5)ncc34)cc2n1C1CCN(C)CC1] | 565 |
| 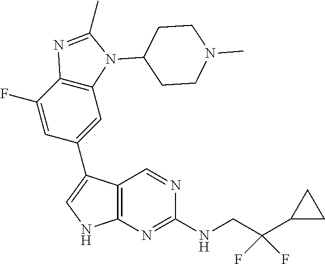 [SMILES: Cc1nc2c(F)cc(-c3c[nH]c4nc(NCC(F)(F)C5CC5)ncc34)cc2n1C1CCN(C)CC1] | 566 |
| 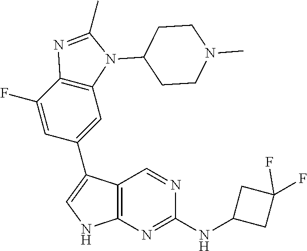 [SMILES: Cc1nc2c(F)cc(-c3c[nH]c4nc(NC5CC(F)(F)C5)ncc34)cc2n1C1CCN(C)CC1] | 567 |
| 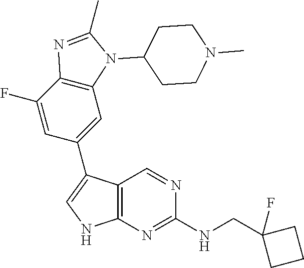 [SMILES: Cc1nc2c(F)cc(-c3c[nH]c4nc(NCC5(F)CCC5)ncc34)cc2n1C1CCN(C)CC1] | 568 |
| 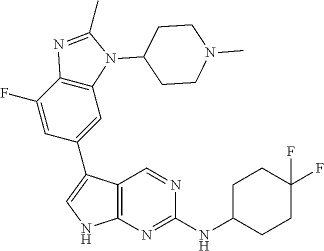 [SMILES: Cc1nc2c(F)cc(-c3c[nH]c4nc(NC5CCC(F)(F)CC5)ncc34)cc2n1C1CCN(C)CC1] | 569 |
| 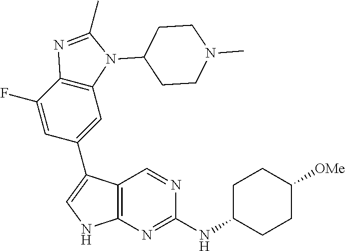 [SMILES: CO[C@H]1CC[C@@H](Nc2ncc3c(-c4cc(F)c5nc(C)n(C6CCN(C)CC6)c5c4)c[nH]c3n2)CC1] | 570 |
| 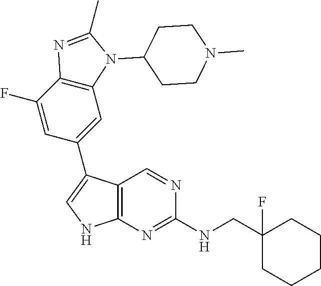 [SMILES: Cc1nc2c(F)cc(-c3c[nH]c4nc(NCC5(F)CCCCC5)ncc34)cc2n1C1CCN(C)CC1] | 571 |
| 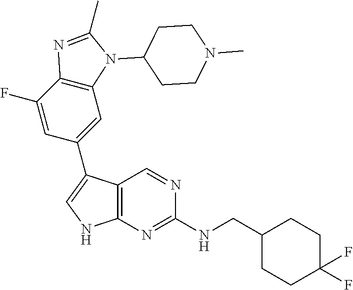 [SMILES: Cc1nc2c(F)cc(-c3c[nH]c4nc(NCC5CCC(F)(F)CC5)ncc34)cc2n1C1CCN(C)CC1] | 572 |
| 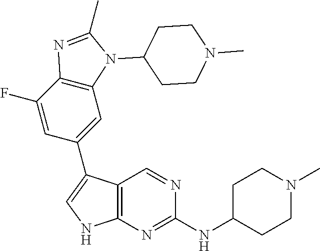 [SMILES: Cc1nc2c(F)cc(-c3c[nH]c4nc(NC5CCN(C)CC5)ncc34)cc2n1C1CCN(C)CC1] | 573 |
| 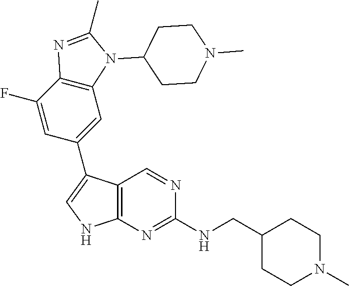 [SMILES: Cc1nc2c(F)cc(-c3c[nH]c4nc(NCC5CCN(C)CC5)ncc34)cc2n1C1CCN(C)CC1] | 574 |
| 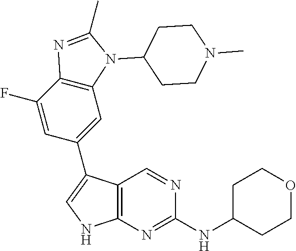 [SMILES: Cc1nc2c(F)cc(-c3c[nH]c4nc(NC5CCOCC5)ncc34)cc2n1C1CCN(C)CC1] | 575 |
| 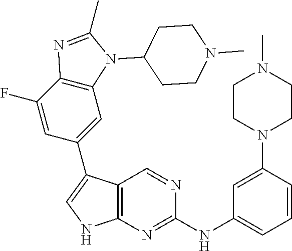 [SMILES: Cc1nc2c(F)cc(-c3c[nH]c4nc(Nc5cccc(N6CCN(C)CC6)c5)ncc34)cc2n1C1CCN(C)CC1] | 576 |
| 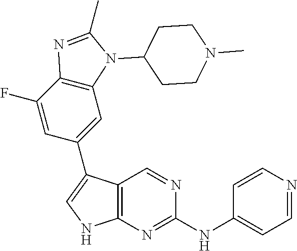 [SMILES: Cc1nc2c(F)cc(-c3c[nH]c4nc(Nc5ccncc5)ncc34)cc2n1C1CCN(C)CC1] | 577 |
| 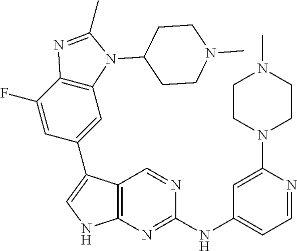 [SMILES: Cc1nc2c(F)cc(-c3c[nH]c4nc(Nc5ccnc(N6CCN(C)CC6)c5)ncc34)cc2n1C1CCN(C)CC1] | 578 |
| 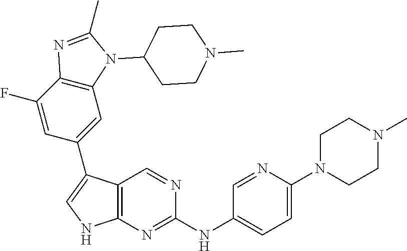 [SMILES: Cc1nc2c(F)cc(-c3c[nH]c4nc(Nc5ccc(N6CCN(C)CC6)nc5)ncc34)cc2n1C1CCN(C)CC1] | 579 |
| 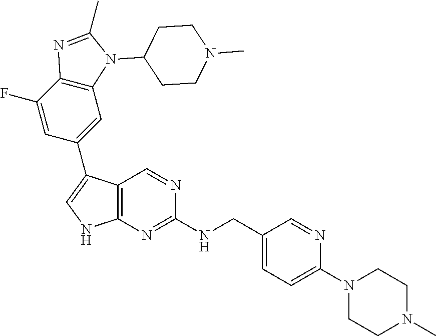 [SMILES: Cc1nc2c(F)cc(-c3c[nH]c4nc(NCc5ccc(N6CCN(C)CC6)nc5)ncc34)cc2n1C1CCN(C)CC1] | 580 |
| 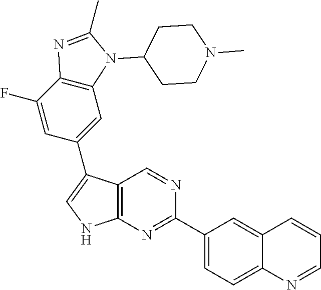 [SMILES: Cc1nc2c(F)cc(-c3c[nH]c4nc(-c5ccc6ncccc6c5)ncc34)cc2n1C1CCN(C)CC1] | 581 |
| 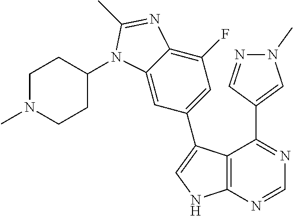 | 582 |
| 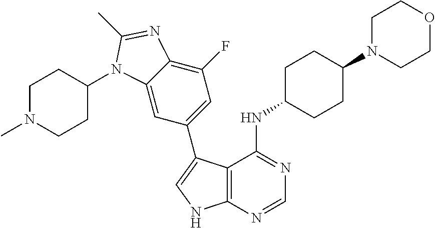 [SMILES: Cc1nc2c(F)cc(-c3c[nH]c4ncnc(N[C@H]5CC[C@H](N6CCOCC6)CC5)c34)cc2n1C1CCN(C)CC1] | 583 |
| 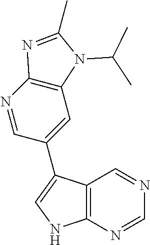 [SMILES: Cc1nc2ncc(-c3c[nH]c4ncncc34)cc2n1C(C)C] | 584 |
| 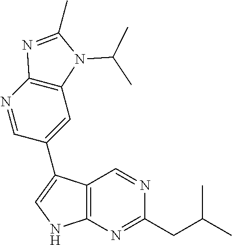 [SMILES: Cc1nc2ncc(-c3c[nH]c4nc(CC(C)C)ncc34)cc2n1C(C)C] | 585 |
| 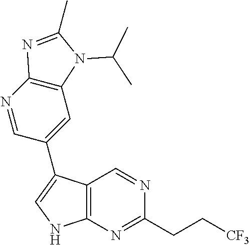 [SMILES: Cc1nc2ncc(-c3c[nH]c4nc(CCC(F)(F)F)ncc34)cc2n1C(C)C] | 586 |
| 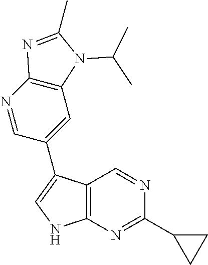 [SMILES: Cc1nc2ncc(-c3c[nH]c4nc(C5CC5)ncc34)cc2n1C(C)C] | 587 |
| 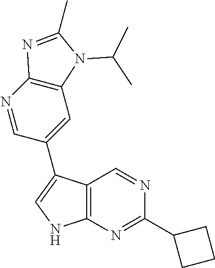 [SMILES: Cc1nc2ncc(-c3c[nH]c4nc(C5CCC5)ncc34)cc2n1C(C)C] | 588 |
| 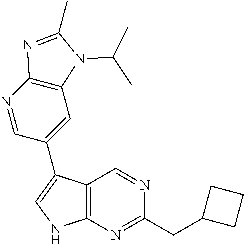 [SMILES: Cc1nc2ncc(-c3c[nH]c4nc(CC5CCC5)ncc34)cc2n1C(C)C] | 589 |
| 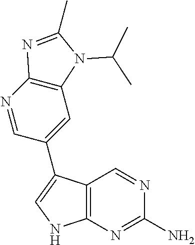 [SMILES: Cc1nc2ncc(-c3c[nH]c4nc(N)ncc34)cc2n1C(C)C] | 590 |
| 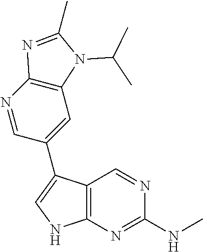 [SMILES: CNc1ncc2c(-c3cnc4nc(C)n(C(C)C)c4c3)c[nH]c2n1] | 591 |
| 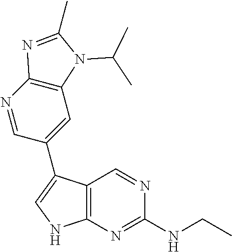 [SMILES: CCNc1ncc2c(-c3cnc4nc(C)n(C(C)C)c4c3)c[nH]c2n1] | 592 |
| 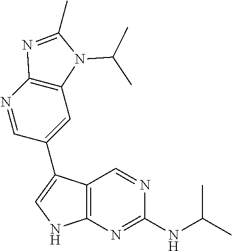 [SMILES: Cc1nc2ncc(-c3c[nH]c4nc(NC(C)C)ncc34)cc2n1C(C)C] | 593 |
| 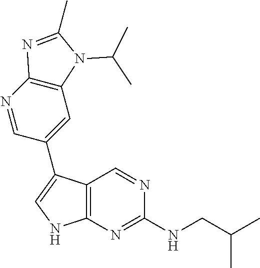 [SMILES: Cc1nc2ncc(-c3c[nH]c4nc(NCC(C)C)ncc34)cc2n1C(C)C] | 594 |
| 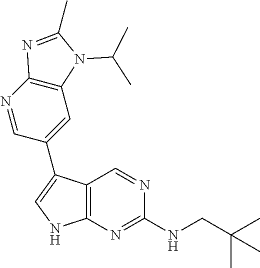 [SMILES: Cc1nc2ncc(-c3c[nH]c4nc(NCC(C)(C)C)ncc34)cc2n1C(C)C] | 595 |
| 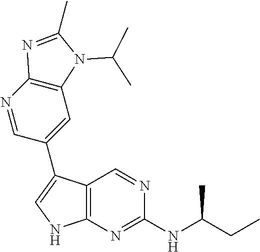 [SMILES: CC[C@H](C)Nc1ncc2c(-c3cnc4nc(C)n(C(C)C)c4c3)c[nH]c2n1] | 596 |
| 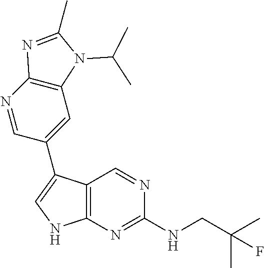 [SMILES: Cc1nc2ncc(-c3c[nH]c4nc(NCC(C)(C)F)ncc34)cc2n1C(C)C] | 597 |
| 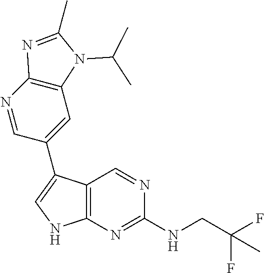 [SMILES: Cc1nc2ncc(-c3c[nH]c4nc(NCC(C)(F)F)ncc34)cc2n1C(C)C] | 598 |
| 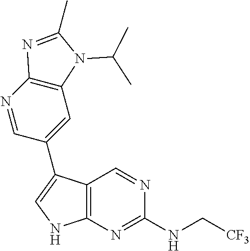 [SMILES: Cc1nc2ncc(-c3c[nH]c4nc(NCC(F)(F)F)ncc34)cc2n1C(C)C] | 599 |
| 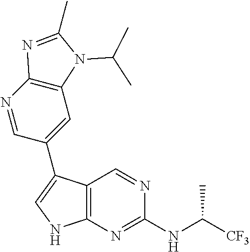 [SMILES: Cc1nc2ncc(-c3c[nH]c4nc(N[C@H](C)C(F)(F)F)ncc34)cc2n1C(C)C] | 600 |
| 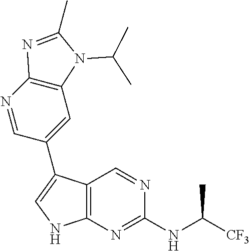 [SMILES: Cc1nc2ncc(-c3c[nH]c4nc(N[C@@H](C)C(F)(F)F)ncc34)cc2n1C(C)C] | 601 |
| 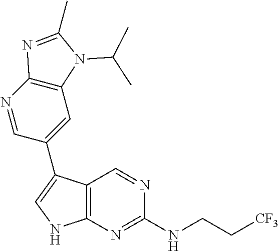 [SMILES: Cc1nc2ncc(-c3c[nH]c4nc(NCCC(F)(F)F)ncc34)cc2n1C(C)C] | 602 |
| 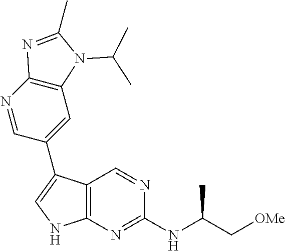 [SMILES: COC[C@H](C)Nc1ncc2c(-c3cnc4nc(C)n(C(C)C)c4c3)c[nH]c2n1] | 603 |
| 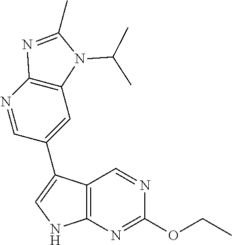 [SMILES: CCOc1ncc2c(-c3cnc4nc(C)n(C(C)C)c4c3)c[nH]c2n1] | 604 |
| 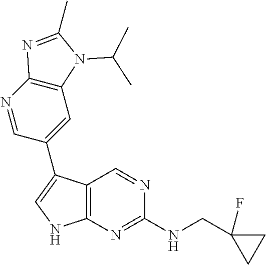 [SMILES: Cc1nc2ncc(-c3c[nH]c4nc(NCC5(F)CC5)ncc34)cc2n1C(C)C] | 605 |
| 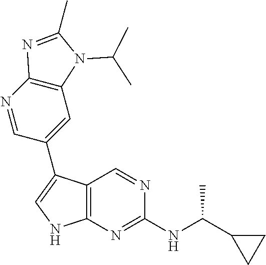 [SMILES: Cc1nc2ncc(-c3c[nH]c4nc(N[C@H](C)C5CC5)ncc34)cc2n1C(C)C] | 606 |
| 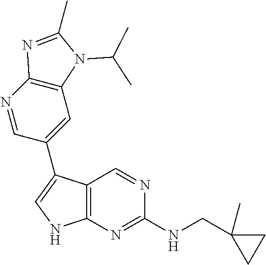 [SMILES: Cc1nc2ncc(-c3c[nH]c4nc(NCC5(C)CC5)ncc34)cc2n1C(C)C] | 607 |
| 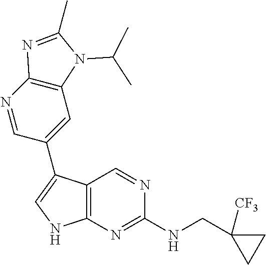 [SMILES: Cc1nc2ncc(-c3c[nH]c4nc(NCC5(C(F)(F)F)CC5)ncc34)cc2n1C(C)C] | 608 |
| 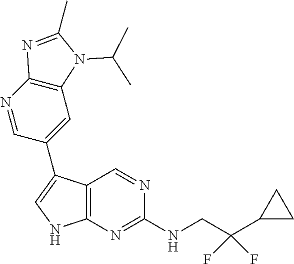 [SMILES: Cc1nc2ncc(-c3c[nH]c4nc(NCC(F)(F)C5CC5)ncc34)cc2n1C(C)C] | 609 |
| 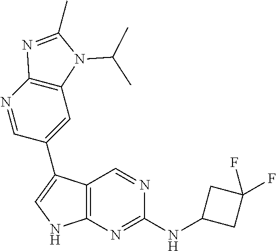 [SMILES: Cc1nc2ncc(-c3c[nH]c4nc(NC5CC(F)(F)C5)ncc34)cc2n1C(C)C] | 610 |
| 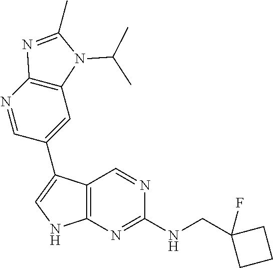 [SMILES: Cc1nc2ncc(-c3c[nH]c4nc(NCC5(F)CCC5)ncc34)cc2n1C(C)C] | 611 |
| 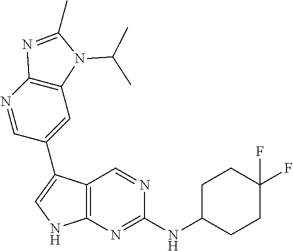 [SMILES: Cc1nc2ncc(-c3c[nH]c4nc(NC5CCC(F)(F)CC5)ncc34)cc2n1C(C)C] | 612 |
| 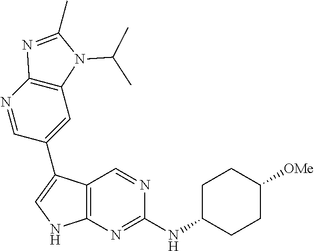 [SMILES: CO[C@H]1CC[C@@H](Nc2ncc3c(-c4cnc5nc(C)n(C(C)C)c5c4)c[nH]c3n2)CC1] | 613 |
| 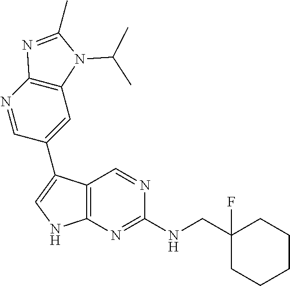 [SMILES: Cc1nc2ncc(-c3c[nH]c4nc(NCC5(F)CCCCC5)ncc34)cc2n1C(C)C] | 614 |
| 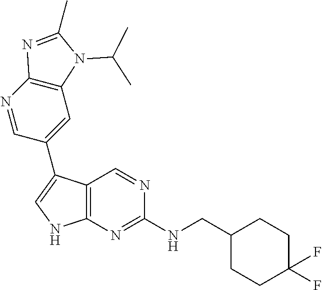 [SMILES: Cc1nc2ncc(-c3c[nH]c4nc(NCC5CCC(F)(F)CC5)ncc34)cc2n1C(C)C] | 615 |
| 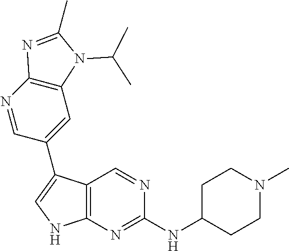 [SMILES: Cc1nc2ncc(-c3c[nH]c4nc(NC5CCN(C)CC5)ncc34)cc2n1C(C)C] | 616 |
| 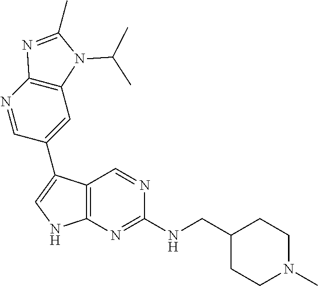 [SMILES: Cc1nc2ncc(-c3c[nH]c4nc(NCC5CCN(C)CC5)ncc34)cc2n1C(C)C] | 617 |
| 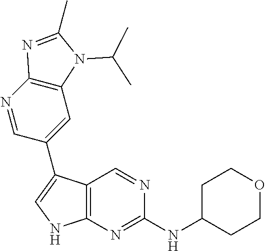 [SMILES: Cc1nc2ncc(-c3c[nH]c4nc(NC5CCOCC5)ncc34)cc2n1C(C)C] | 618 |
| 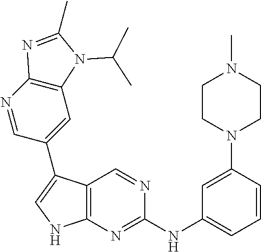 [SMILES: Cc1nc2ncc(-c3c[nH]c4nc(Nc5cccc(N6CCN(C)CC6)c5)ncc34)cc2n1C(C)C] | 619 |
| 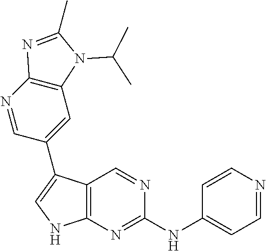 [SMILES: Cc1nc2ncc(-c3c[nH]c4nc(Nc5ccncc5)ncc34)cc2n1C(C)C] | 620 |
| 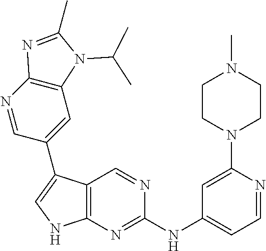 [SMILES: Cc1nc2ncc(-c3c[nH]c4nc(Nc5ccnc(N6CCN(C)CC6)c5)ncc34)cc2n1C(C)C] | 621 |
| 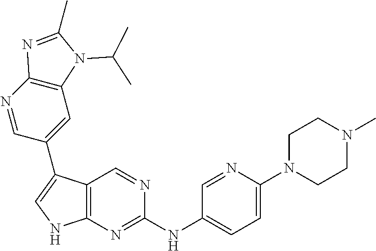 [SMILES: Cc1nc2ncc(-c3c[nH]c4nc(Nc5ccc(N6CCN(C)CC6)nc5)ncc34)cc2n1C(C)C] | 622 |
| 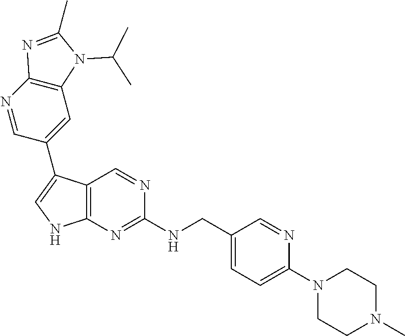 [SMILES: Cc1nc2ncc(-c3c[nH]c4nc(NCc5ccc(N6CCN(C)CC6)nc5)ncc34)cc2n1C(C)C] | 623 |
| 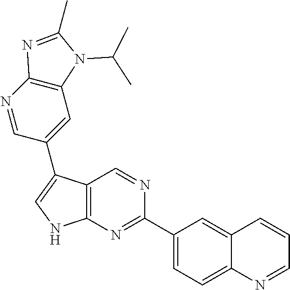 [SMILES: Cc1nc2ncc(-c3c[nH]c4nc(-c5ccc6ncccc6c5)ncc34)cc2n1C(C)C] | 624 |
| 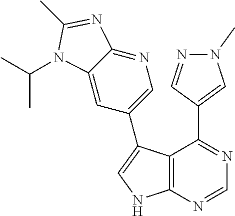 [SMILES: Cc1nc2ncc(-c3c[nH]c4ncnc(-c5cnn(C)c5)c34)cc2n1C(C)C] | 625 |
| 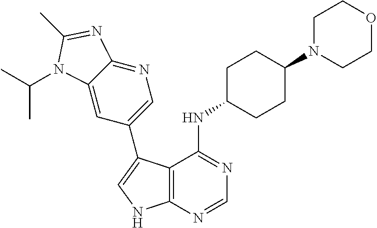 [SMILES: Cc1nc2ncc(-c3c[nH]c4ncnc(N[C@H]5CC[C@H](N6CCOCC6)CC5)c34)cc2n1C(C)C] | 626 |
| 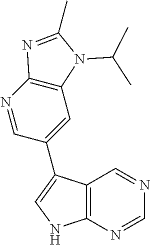 [SMILES: Cc1nc2ncc(-c3c[nH]c4ncncc34)cc2n1C(C)C] | 627 |
| 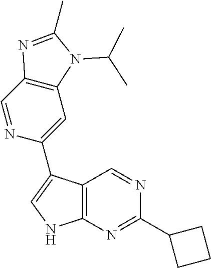 [SMILES: Cc1nc2cnc(-c3c[nH]c4nc(C5CCC5)ncc34)cc2n1C(C)C] | 628 |
| 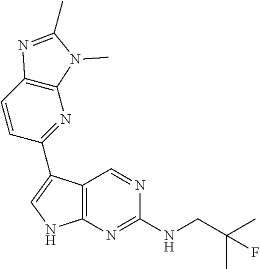 [SMILES: Cc1nc2ccc(-c3c[nH]c4nc(NCC(C)(C)F)ncc34)nc2n1C] | 629 |
| 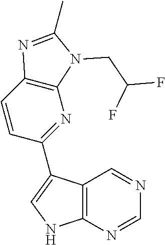 [SMILES: Cc1nc2ccc(-c3c[nH]c4ncncc34)nc2n1CC(F)F] | 630 |
| 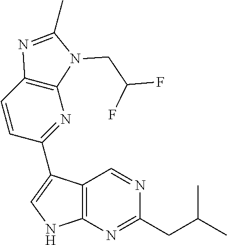 [SMILES: Cc1nc2ccc(-c3c[nH]c4nc(CC(C)C)ncc34)nc2n1CC(F)F] | 631 |
| 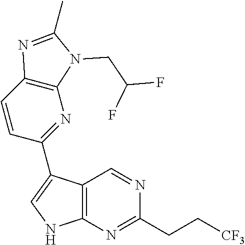 [SMILES: Cc1nc2ccc(-c3c[nH]c4nc(CCC(F)(F)F)ncc34)nc2n1CC(F)F] | 632 |
| 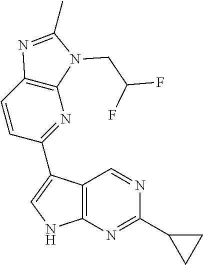 [SMILES: Cc1nc2ccc(-c3c[nH]c4nc(C5CC5)ncc34)nc2n1CC(F)F] | 633 |
| 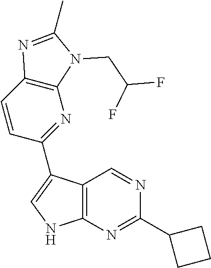 [SMILES: Cc1nc2ccc(-c3c[nH]c4nc(C5CCC5)ncc34)nc2n1CC(F)F] | 634 |
| 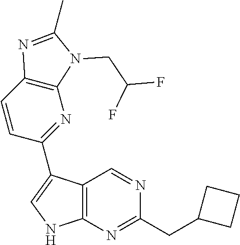 [SMILES: Cc1nc2ccc(-c3c[nH]c4nc(CC5CCC5)ncc34)nc2n1CC(F)F] | 635 |
| 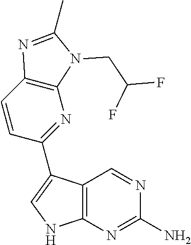 [SMILES: Cc1nc2ccc(-c3c[nH]c4nc(N)ncc34)nc2n1CC(F)F] | 636 |
| 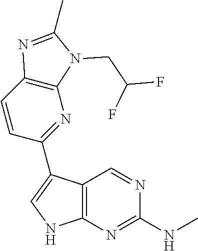 [SMILES: CNc1ncc2c(-c3ccc4nc(C)n(CC(F)F)c4n3)c[nH]c2n1] | 637 |
| 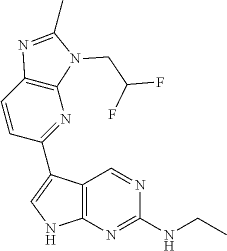 [SMILES: CCNc1ncc2c(-c3ccc4nc(C)n(CC(F)F)c4n3)c[nH]c2n1] | 638 |
| 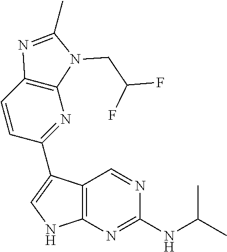 [SMILES: Cc1nc2ccc(-c3c[nH]c4nc(NC(C)C)ncc34)nc2n1CC(F)F] | 639 |
| 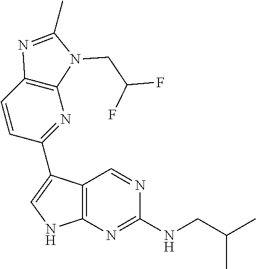 [SMILES: Cc1nc2ccc(-c3c[nH]c4nc(NCC(C)C)ncc34)nc2n1CC(F)F] | 640 |
| 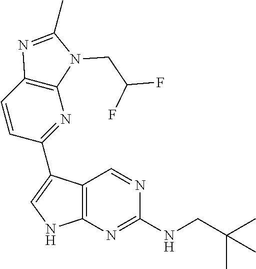 [SMILES: Cc1nc2ccc(-c3c[nH]c4nc(NCC(C)(C)C)ncc34)nc2n1CC(F)F] | 641 |
| 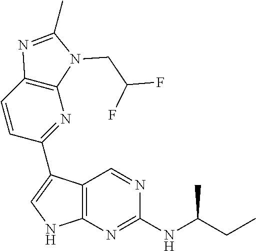 [SMILES: CC[C@H](C)Nc1ncc2c(-c3ccc4nc(C)n(CC(F)F)c4n3)c[nH]c2n1] | 642 |
| 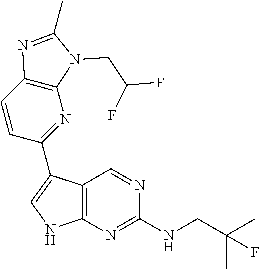 [SMILES: Cc1nc2ccc(-c3c[nH]c4nc(NCC(C)(C)F)ncc34)nc2n1CC(F)F] | 643 |
| 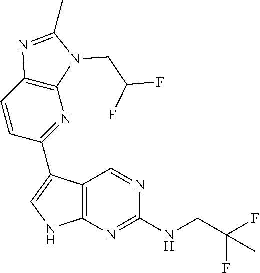 [SMILES: Cc1nc2ccc(-c3c[nH]c4nc(NCC(C)(F)F)ncc34)nc2n1CC(F)F] | 644 |
| 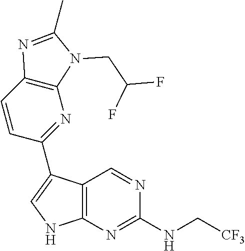 [SMILES: Cc1nc2ccc(-c3c[nH]c4nc(NCC(F)(F)F)ncc34)nc2n1CC(F)F] | 645 |
| 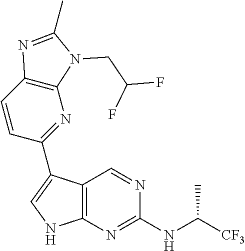 [SMILES: Cc1nc2ccc(-c3c[nH]c4nc(N[C@H](C)C(F)(F)F)ncc34)nc2n1CC(F)F] | 646 |
| 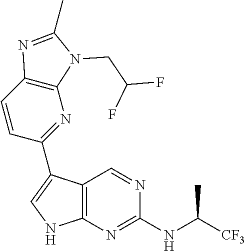 [SMILES: Cc1nc2ccc(-c3c[nH]c4nc(N[C@@H](C)C(F)(F)F)ncc34)nc2n1CC(F)F] | 647 |
| 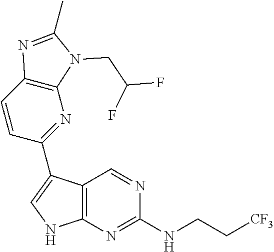 [SMILES: Cc1nc2ccc(-c3c[nH]c4nc(NCCC(F)(F)F)ncc34)nc2n1CC(F)F] | 648 |
| 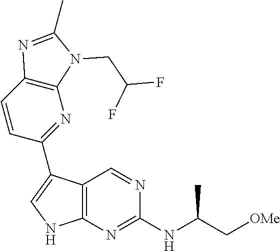 [SMILES: COC[C@H](C)Nc1ncc2c(-c3ccc4nc(C)n(CC(F)F)c4n3)c[nH]c2n1] | 649 |
| 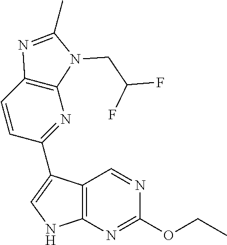 [SMILES: CCOc1ncc2c(-c3ccc4nc(C)n(CC(F)F)c4n3)c[nH]c2n1] | 650 |
| 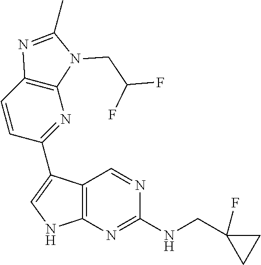 [SMILES: Cc1nc2ccc(-c3c[nH]c4nc(NCC5(F)CC5)ncc34)nc2n1CC(F)F] | 651 |
| 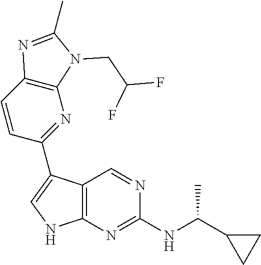 [SMILES: Cc1nc2ccc(-c3c[nH]c4nc(N[C@H](C)C5CC5)ncc34)nc2n1CC(F)F] | 652 |
| 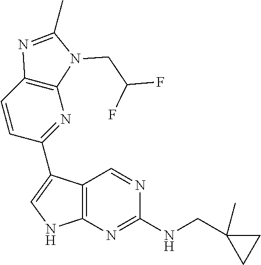 [SMILES: Cc1nc2ccc(-c3c[nH]c4nc(NCC5(C)CC5)ncc34)nc2n1CC(F)F] | 653 |
| 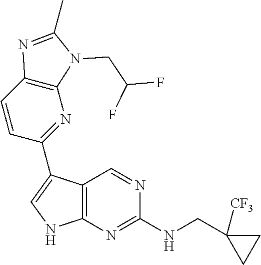 [SMILES: Cc1nc2ccc(-c3c[nH]c4nc(NCC5(C(F)(F)F)CC5)ncc34)nc2n1CC(F)F] | 654 |
| 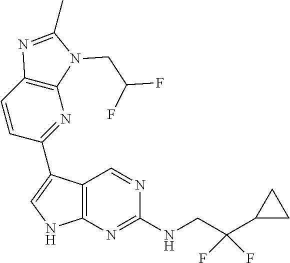 [SMILES: Cc1nc2ccc(-c3c[nH]c4nc(NCC(F)(F)C5CC5)ncc34)nc2n1CC(F)F] | 655 |
| 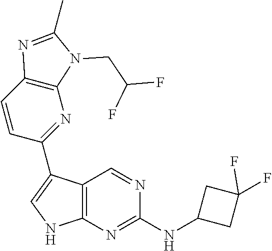 [SMILES: Cc1nc2ccc(-c3c[nH]c4nc(NC5CC(F)(F)C5)ncc34)nc2n1CC(F)F] | 656 |
| 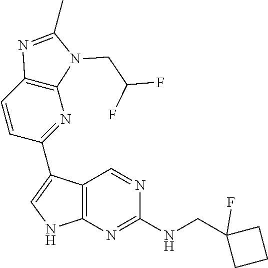 [SMILES: Cc1nc2ccc(-c3c[nH]c4nc(NCC5(F)CCC5)ncc34)nc2n1CC(F)F] | 657 |
| 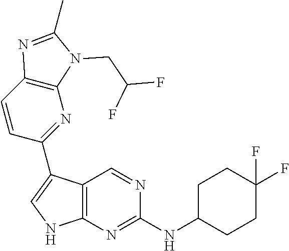 [SMILES: Cc1nc2ccc(-c3c[nH]c4nc(NC5CCC(F)(F)CC5)ncc34)nc2n1CC(F)F] | 658 |
| 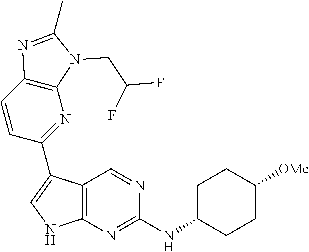 [SMILES: CO[C@H]1CC[C@@H](Nc2ncc3c(-c4ccc5nc(C)n(CC(F)F)c5n4)c[nH]c3n2)CC1] | 659 |
| 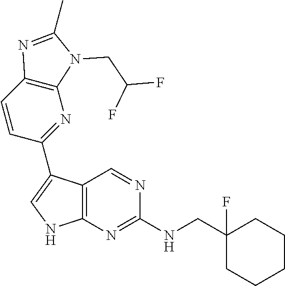 [SMILES: Cc1nc2ccc(-c3c[nH]c4nc(NCC5(F)CCCCC5)ncc34)nc2n1CC(F)F] | 660 |
| 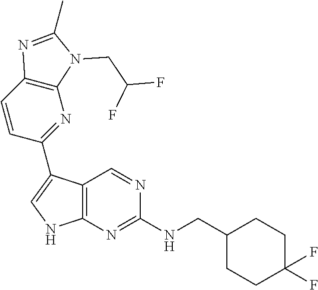 [SMILES: Cc1nc2ccc(-c3c[nH]c4nc(NCC5CCC(F)(F)CC5)ncc34)nc2n1CC(F)F] | 661 |
| 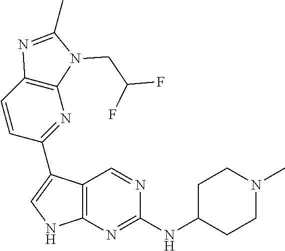 [SMILES: Cc1nc2ccc(-c3c[nH]c4nc(NC5CCN(C)CC5)ncc34)nc2n1CC(F)F] | 662 |
| 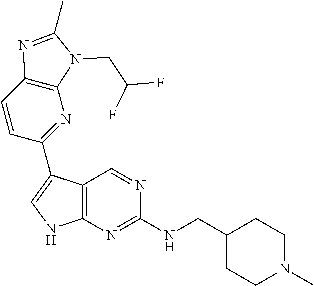 [SMILES: Cc1nc2ccc(-c3c[nH]c4nc(NCC5CCN(C)CC5)ncc34)nc2n1CC(F)F] | 663 |
| 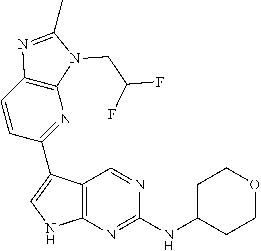 [SMILES: Cc1nc2ccc(-c3c[nH]c4nc(NC5CCOCC5)ncc34)nc2n1CC(F)F] | 664 |
| 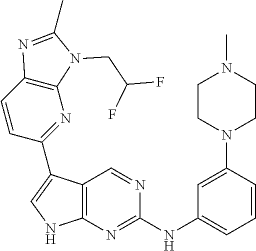 [SMILES: Cc1nc2ccc(-c3c[nH]c4nc(Nc5cccc(N6CCN(C)CC6)c5)ncc34)nc2n1CC(F)F] | 665 |
| 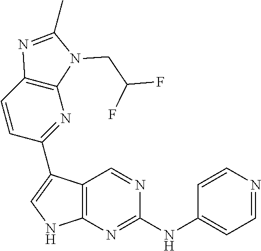 [SMILES: Cc1nc2ccc(-c3c[nH]c4nc(Nc5ccncc5)ncc34)nc2n1CC(F)F] | 666 |
| 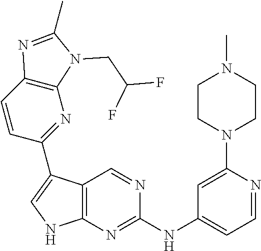 [SMILES: Cc1nc2ccc(-c3c[nH]c4nc(Nc5ccnc(N6CCN(C)CC6)c5)ncc34)nc2n1CC(F)F] | 667 |
| 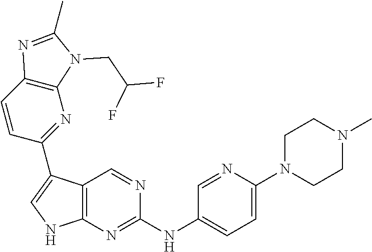 [SMILES: Cc1nc2ccc(-c3c[nH]c4nc(Nc5ccc(N6CCN(C)CC6)nc5)ncc34)nc2n1CC(F)F] | 668 |
| 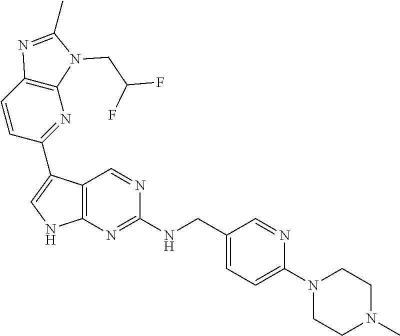 [SMILES: Cc1nc2ccc(-c3c[nH]c4nc(NCc5ccc(N6CCN(C)CC6)nc5)ncc34)nc2n1CC(F)F] | 669 |
| 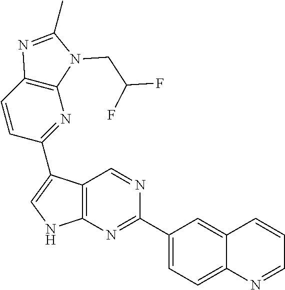 [SMILES: Cc1nc2ccc(-c3c[nH]c4nc(-c5ccc6ncccc6c5)ncc34)nc2n1CC(F)F] | 670 |
| 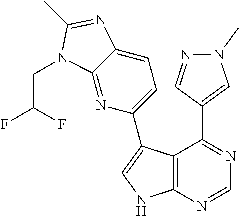 [SMILES: Cc1nc2ccc(-c3c[nH]c4ncnc(-c5cnn(C)c5)c34)nc2n1CC(F)F] | 671 |
| 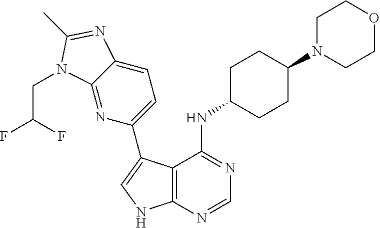 [SMILES: Cc1nc2ccc(-c3c[nH]c4ncnc(N[C@H]5CC[C@H](N6CCOCC6)CC5)c34)nc2n1CC(F)F] | 672 |
| 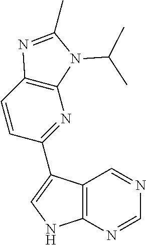 [SMILES: Cc1nc2ccc(-c3c[nH]c4ncncc34)nc2n1C(C)C] | 673 |
| 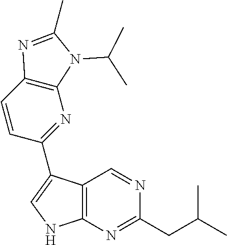 [SMILES: Cc1nc2ccc(-c3c[nH]c4nc(CC(C)C)ncc34)nc2n1C(C)C] | 674 |
| 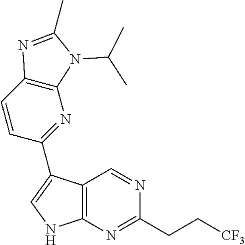 [SMILES: Cc1nc2ccc(-c3c[nH]c4nc(CCC(F)(F)F)ncc34)nc2n1C(C)C] | 675 |
| 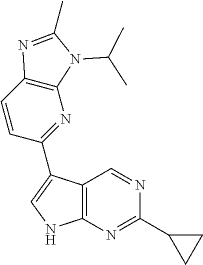 [SMILES: Cc1nc2ccc(-c3c[nH]c4nc(C5CC5)ncc34)nc2n1C(C)C] | 676 |
| 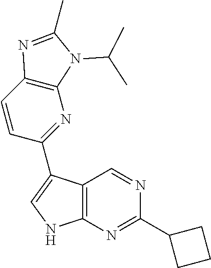 [SMILES: Cc1nc2ccc(-c3c[nH]c4nc(C5CCC5)ncc34)nc2n1C(C)C] | 677 |
| 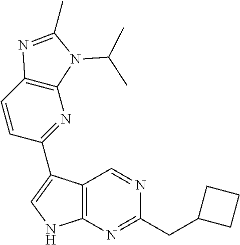 [SMILES: Cc1nc2ccc(-c3c[nH]c4nc(CC5CCC5)ncc34)nc2n1C(C)C] | 678 |
| 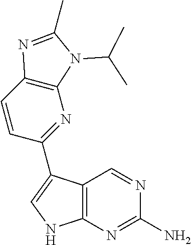 [SMILES: Cc1nc2ccc(-c3c[nH]c4nc(N)ncc34)nc2n1C(C)C] | 679 |
| 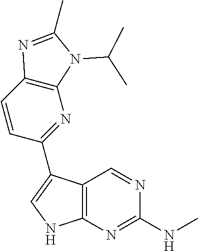 [SMILES: CNc1ncc2c(-c3ccc4nc(C)n(C(C)C)c4n3)c[nH]c2n1] | 680 |
| 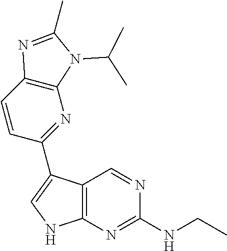 [SMILES: CCNc1ncc2c(-c3ccc4nc(C)n(C(C)C)c4n3)c[nH]c2n1] | 681 |
| 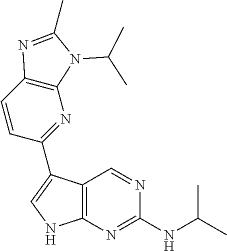 [SMILES: Cc1nc2ccc(-c3c[nH]c4nc(NC(C)C)ncc34)nc2n1C(C)C] | 682 |
| 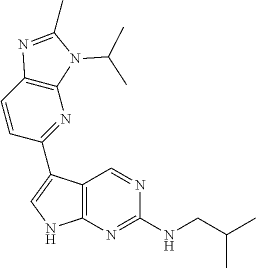 [SMILES: Cc1nc2ccc(-c3c[nH]c4nc(NCC(C)C)ncc34)nc2n1C(C)C] | 683 |
| 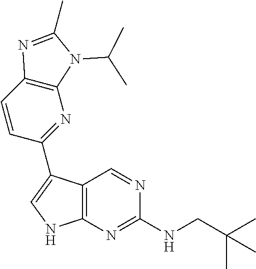 [SMILES: Cc1nc2ccc(-c3c[nH]c4nc(NCC(C)(C)C)ncc34)nc2n1C(C)C] | 684 |
| 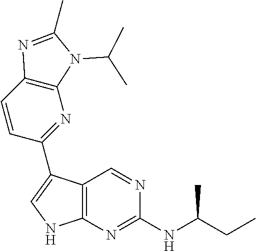 [SMILES: CC[C@H](C)Nc1ncc2c(-c3ccc4nc(C)n(C(C)C)c4n3)c[nH]c2n1] | 685 |
| 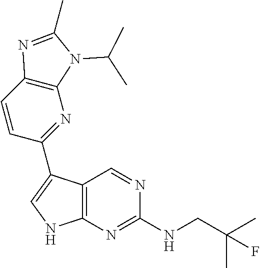 [SMILES: Cc1nc2ccc(-c3c[nH]c4nc(NCC(C)(C)F)ncc34)nc2n1C(C)C] | 686 |
| 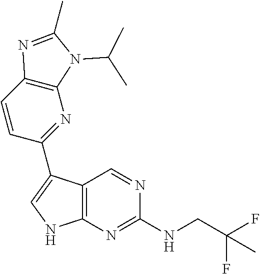 [SMILES: Cc1nc2ccc(-c3c[nH]c4nc(NCC(C)(F)F)ncc34)nc2n1C(C)C] | 687 |
| 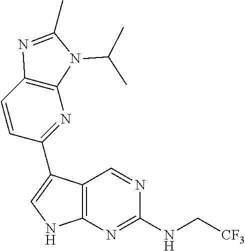 [SMILES: Cc1nc2ccc(-c3c[nH]c4nc(NCC(F)(F)F)ncc34)nc2n1C(C)C] | 688 |
| 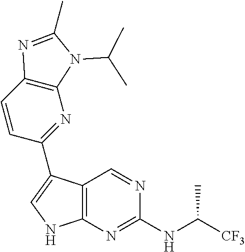 [SMILES: Cc1nc2ccc(-c3c[nH]c4nc(N[C@H](C)C(F)(F)F)ncc34)nc2n1C(C)C] | 689 |
| 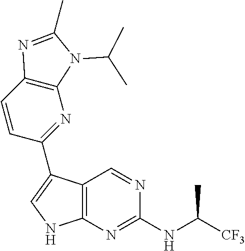 [SMILES: Cc1nc2ccc(-c3c[nH]c4nc(N[C@@H](C)C(F)(F)F)ncc34)nc2n1C(C)C] | 690 |
| 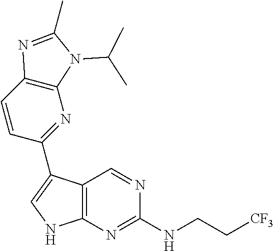 [SMILES: Cc1nc2ccc(-c3c[nH]c4nc(NCCC(F)(F)F)ncc34)nc2n1C(C)C] | 691 |
| 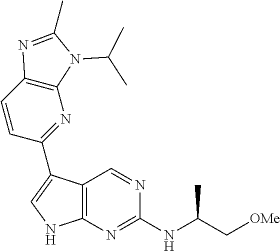 [SMILES: COC[C@H](C)Nc1ncc2c(-c3ccc4nc(C)n(C(C)C)c4n3)c[nH]c2n1] | 692 |
| 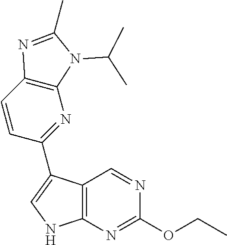 [SMILES: CCOc1ncc2c(-c3ccc4nc(C)n(C(C)C)c4n3)c[nH]c2n1] | 693 |
| 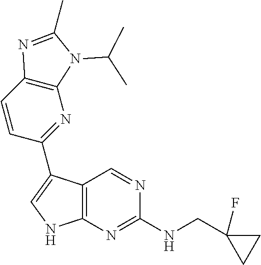 [SMILES: Cc1nc2ccc(-c3c[nH]c4nc(NCC5(F)CC5)ncc34)nc2n1C(C)C] | 694 |
| 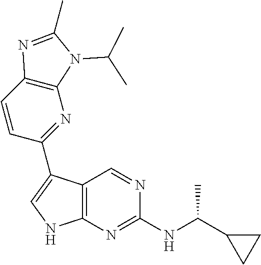 [SMILES: Cc1nc2ccc(-c3c[nH]c4nc(N[C@H](C)C5CC5)ncc34)nc2n1C(C)C] | 695 |
| 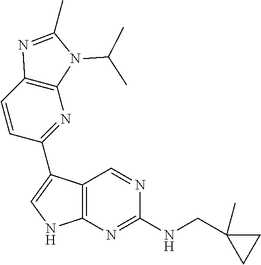 [SMILES: Cc1nc2ccc(-c3c[nH]c4nc(NCC5(C)CC5)ncc34)nc2n1C(C)C] | 696 |
| 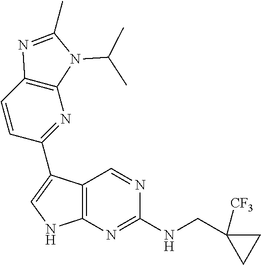 [SMILES: Cc1nc2ccc(-c3c[nH]c4nc(NCC5(C(F)(F)F)CC5)ncc34)nc2n1C(C)C] | 697 |
| 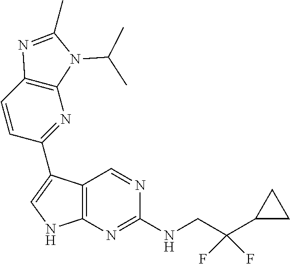 [SMILES: Cc1nc2ccc(-c3c[nH]c4nc(NCC(F)(F)C5CC5)ncc34)nc2n1C(C)C] | 698 |
| 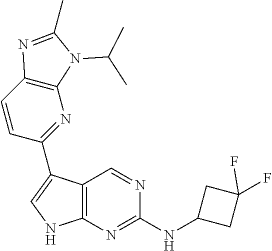 [SMILES: Cc1nc2ccc(-c3c[nH]c4nc(NC5CC(F)(F)C5)ncc34)nc2n1C(C)C] | 699 |
| 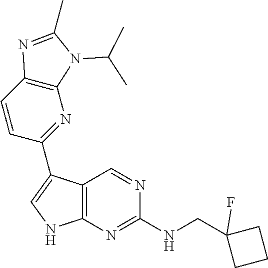 [SMILES: Cc1nc2ccc(-c3c[nH]c4nc(NCC5(F)CCC5)ncc34)nc2n1C(C)C] | 700 |
| 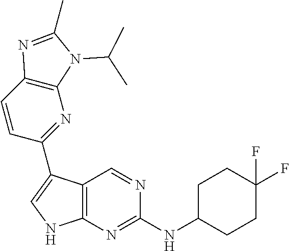 [SMILES: Cc1nc2ccc(-c3c[nH]c4nc(NC5CCC(F)(F)CC5)ncc34)nc2n1C(C)C] | 701 |
| 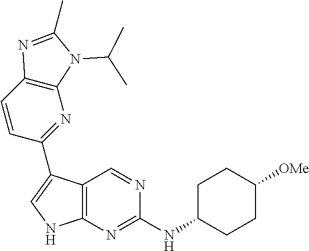 [SMILES: CO[C@H]1CC[C@@H](Nc2ncc3c(-c4ccc5nc(C)n(C(C)C)c5n4)c[nH]c3n2)CC1] | 702 |
| 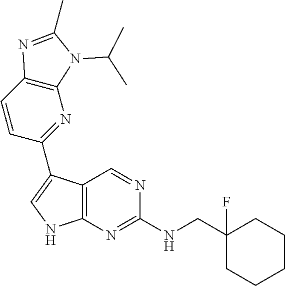 [SMILES: Cc1nc2ccc(-c3c[nH]c4nc(NCC5(F)CCCCC5)ncc34)nc2n1C(C)C] | 703 |
| 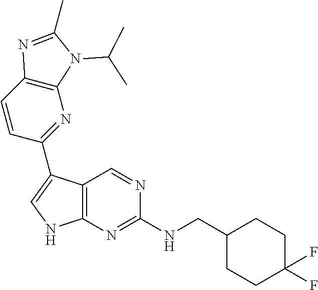 [SMILES: Cc1nc2ccc(-c3c[nH]c4nc(NCC5CCC(F)(F)CC5)ncc34)nc2n1C(C)C] | 704 |
| 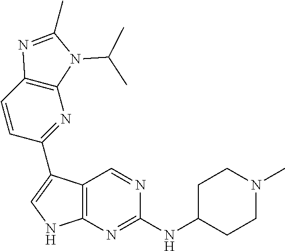 [SMILES: Cc1nc2ccc(-c3c[nH]c4nc(NC5CCN(C)CC5)ncc34)nc2n1C(C)C] | 705 |
| 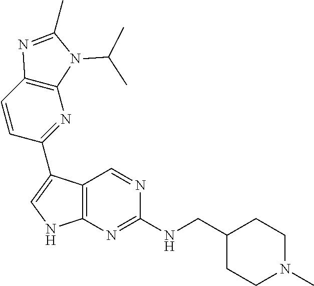 [SMILES: Cc1nc2ccc(-c3c[nH]c4nc(NCC5CCN(C)CC5)ncc34)nc2n1C(C)C] | 706 |
| 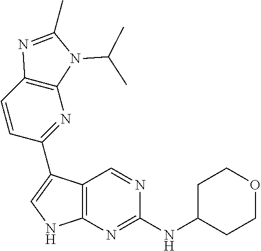 [SMILES: Cc1nc2ccc(-c3c[nH]c4nc(NC5CCOCC5)ncc34)nc2n1C(C)C] | 707 |
| 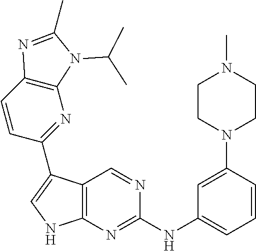 [SMILES: Cc1nc2ccc(-c3c[nH]c4nc(Nc5cccc(N6CCN(C)CC6)c5)ncc34)nc2n1C(C)C] | 708 |
| 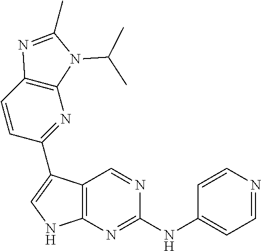 [SMILES: Cc1nc2ccc(-c3c[nH]c4nc(Nc5ccncc5)ncc34)nc2n1C(C)C] | 709 |
| 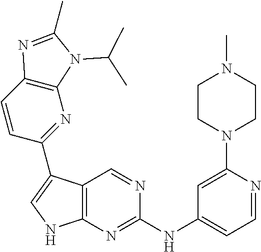 [SMILES: Cc1nc2ccc(-c3c[nH]c4nc(Nc5ccnc(N6CCN(C)CC6)c5)ncc34)nc2n1C(C)C] | 710 |
| 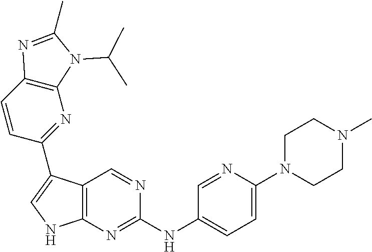 [SMILES: Cc1nc2ccc(-c3c[nH]c4nc(Nc5ccc(N6CCN(C)CC6)nc5)ncc34)nc2n1C(C)C] | 711 |
| 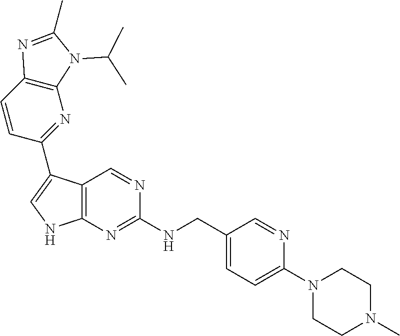 [SMILES: Cc1nc2ccc(-c3c[nH]c4nc(NCc5ccc(N6CCN(C)CC6)nc5)ncc34)nc2n1C(C)C] | 712 |
| 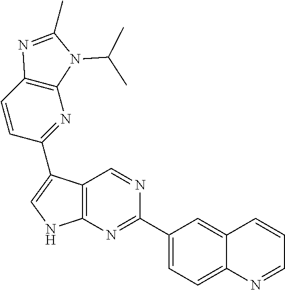 [SMILES: Cc1nc2ccc(-c3c[nH]c4nc(-c5ccc6ncccc6c5)ncc34)nc2n1C(C)C] | 713 |
| 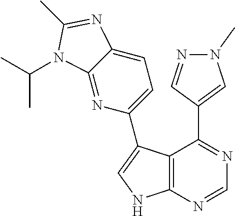 [SMILES: Cc1nc2ccc(-c3c[nH]c4ncnc(-c5cnn(C)c5)c34)nc2n1C(C)C] | 714 |
| 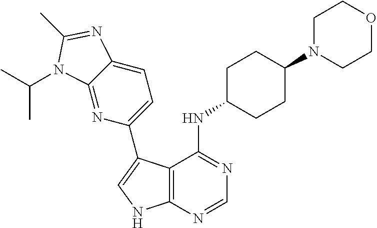 [SMILES: Cc1nc2ccc(-c3c[nH]c4ncnc(N[C@H]5CC[C@H](N6CCOCC6)CC5)c34)nc2n1C(C)C] | 715 |
| 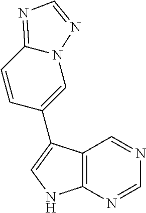 [SMILES: c1ncc2c(-c3ccc4ncnn4c3)c[nH]c2n1] | 716 |
| 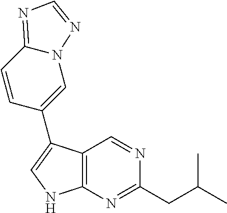 [SMILES: CC(C)Cc1ncc2c(-c3ccc4ncnn4c3)c[nH]c2n1] | 717 |
| 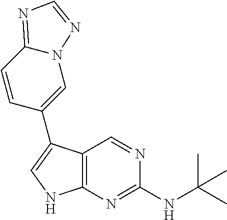 [SMILES: CC(C)(C)Nc1ncc2c(-c3ccc4ncnn4c3)c[nH]c2n1] | 718 |
| 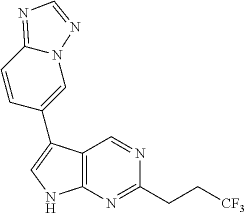 [SMILES: FC(F)(F)CCc1ncc2c(-c3ccc4ncnn4c3)c[nH]c2n1] | 719 |
| 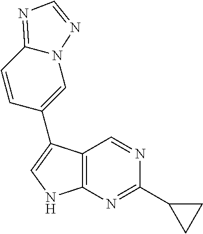 [SMILES: c1nc2ccc(-c3c[nH]c4nc(C5CC5)ncc34)cn2n1] | 720 |
| 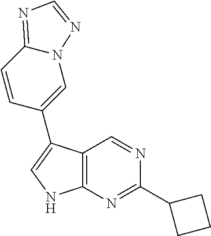 [SMILES: c1nc2ccc(-c3c[nH]c4nc(C5CCC5)ncc34)cn2n1] | 721 |
| 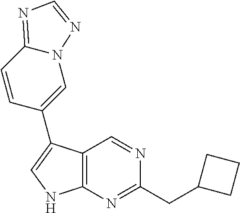 [SMILES: c1nc2ccc(-c3c[nH]c4nc(CC5CCC5)ncc34)cn2n1] | 722 |
| 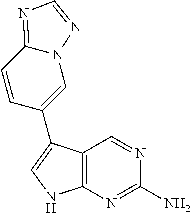 [SMILES: Nc1ncc2c(-c3ccc4ncnn4c3)c[nH]c2n1] | 723 |
| 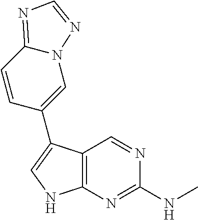 [SMILES: CNc1ncc2c(-c3ccc4ncnn4c3)c[nH]c2n1] | 724 |
| 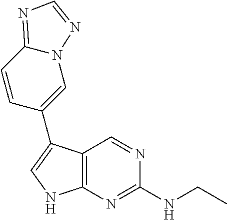 [SMILES: CCNc1ncc2c(-c3ccc4ncnn4c3)c[nH]c2n1] | 725 |
| 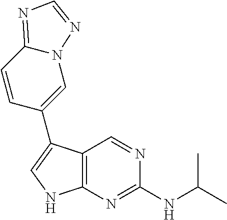 [SMILES: CC(C)Nc1ncc2c(-c3ccc4ncnn4c3)c[nH]c2n1] | 726 |
| 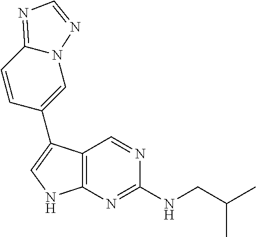 [SMILES: CC(C)CNc1ncc2c(-c3ccc4ncnn4c3)c[nH]c2n1] | 727 |
| 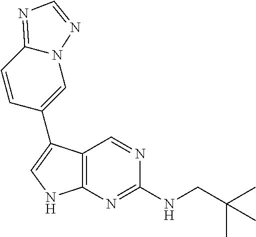 [SMILES: CC(C)(C)CNc1ncc2c(-c3ccc4ncnn4c3)c[nH]c2n1] | 728 |
| 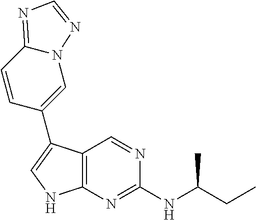 [SMILES: CC[C@H](C)Nc1ncc2c(-c3ccc4ncnn4c3)c[nH]c2n1] | 729 |
| 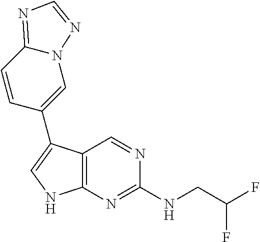 [SMILES: FC(F)CNc1ncc2c(-c3ccc4ncnn4c3)c[nH]c2n1] | 730 |
| 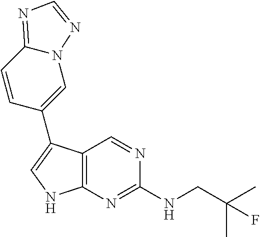 [SMILES: CC(C)(F)CNc1ncc2c(-c3ccc4ncnn4c3)c[nH]c2n1] | 731 |
| 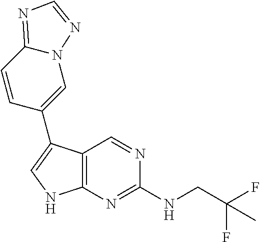 [SMILES: CC(F)(F)CNc1ncc2c(-c3ccc4ncnn4c3)c[nH]c2n1] | 732 |
| 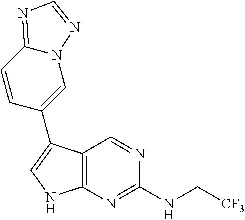 [SMILES: FC(F)(F)CNc1ncc2c(-c3ccc4ncnn4c3)c[nH]c2n1] | 733 |
| 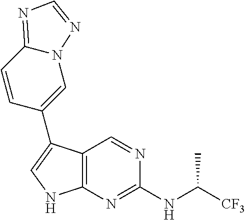 [SMILES: C[C@@H](Nc1ncc2c(-c3ccc4ncnn4c3)c[nH]c2n1)C(F)(F)F] | 734 |
| 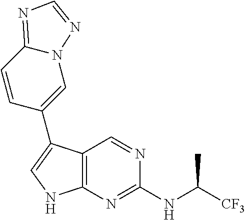 [SMILES: C[C@H](Nc1ncc2c(-c3ccc4ncnn4c3)c[nH]c2n1)C(F)(F)F] | 735 |
| 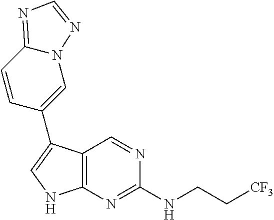 [SMILES: FC(F)(F)CCNc1ncc2c(-c3ccc4ncnn4c3)c[nH]c2n1] | 736 |
| 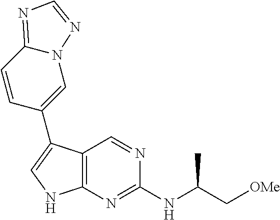 [SMILES: COC[C@H](C)Nc1ncc2c(-c3ccc4ncnn4c3)c[nH]c2n1] | 737 |
| 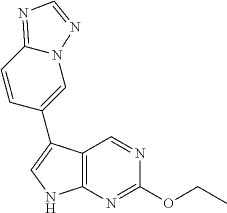 [SMILES: CCOc1ncc2c(-c3ccc4ncnn4c3)c[nH]c2n1] | 738 |
| 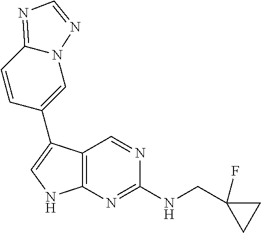 [SMILES: FC1(CNc2ncc3c(-c4ccc5ncnn5c4)c[nH]c3n2)CC1] | 739 |
| 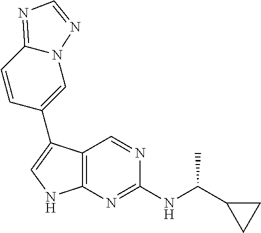 [SMILES: C[C@@H](Nc1ncc2c(-c3ccc4ncnn4c3)c[nH]c2n1)C1CC1] | 740 |
| 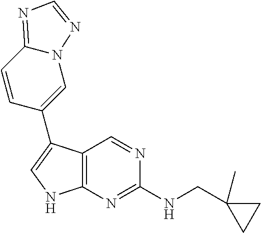 [SMILES: CC1(CNc2ncc3c(-c4ccc5ncnn5c4)c[nH]c3n2)CC1] | 741 |
| 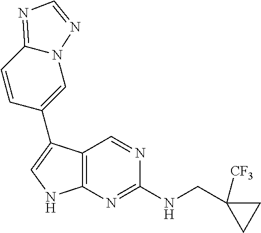 [SMILES: FC(F)(F)C1(CNc2ncc3c(-c4ccc5ncnn5c4)c[nH]c3n2)CC1] | 742 |
| 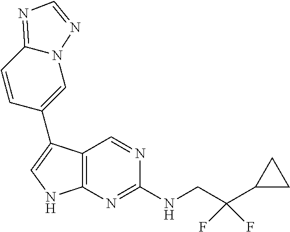 [SMILES: FC(F)(CNc1ncc2c(-c3ccc4ncnn4c3)c[nH]c2n1)C1CC1] | 743 |
| 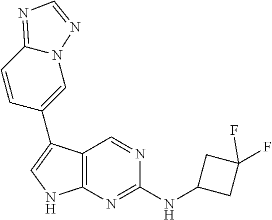 [SMILES: FC1(F)CC(Nc2ncc3c(-c4ccc5ncnn5c4)c[nH]c3n2)C1] | 744 |
| 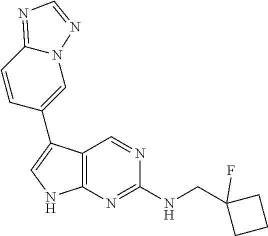 [SMILES: FC1(CNc2ncc3c(-c4ccc5ncnn5c4)c[nH]c3n2)CCC1] | 745 |
| 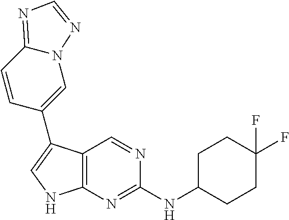 [SMILES: FC1(F)CCC(Nc2ncc3c(-c4ccc5ncnn5c4)c[nH]c3n2)CC1] | 746 |
| 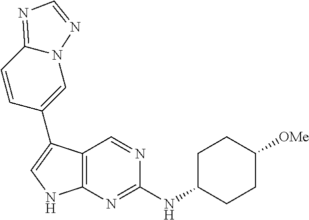 [SMILES: CO[C@H]1CC[C@@H](Nc2ncc3c(-c4ccc5ncnn5c4)c[nH]c3n2)CC1] | 747 |
| 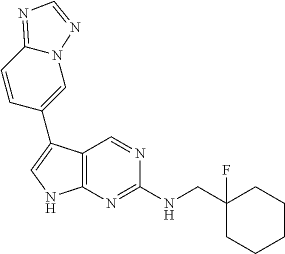 [SMILES: FC1(CNc2ncc3c(-c4ccc5ncnn5c4)c[nH]c3n2)CCCCC1] | 748 |
| 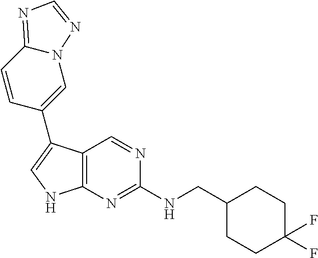 [SMILES: FC1(F)CCC(CNc2ncc3c(-c4ccc5ncnn5c4)c[nH]c3n2)CC1] | 749 |
| 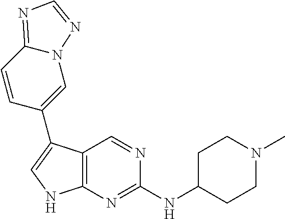 [SMILES: CN1CCC(Nc2ncc3c(-c4ccc5ncnn5c4)c[nH]c3n2)CC1] | 750 |
| 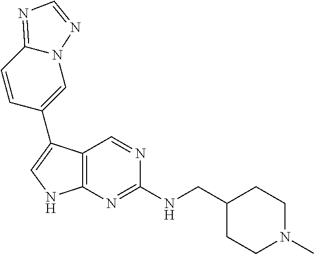 [SMILES: CN1CCC(CNc2ncc3c(-c4ccc5ncnn5c4)c[nH]c3n2)CC1] | 751 |
| 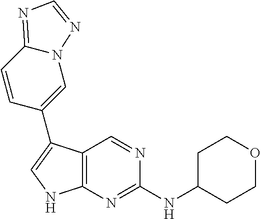 [SMILES: c1nc2ccc(-c3c[nH]c4nc(NC5CCOCC5)ncc34)cn2n1] | 752 |
| 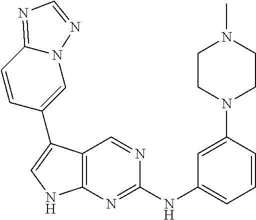 [SMILES: CN1CCN(c2cccc(Nc3ncc4c(-c5ccc6ncnn6c5)c[nH]c4n3)c2)CC1] | 753 |
| 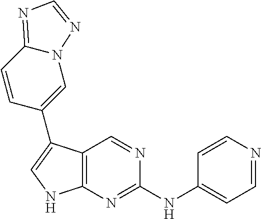 [SMILES: c1cc(Nc2ncc3c(-c4ccc5ncnn5c4)c[nH]c3n2)ccn1] | 754 |
| 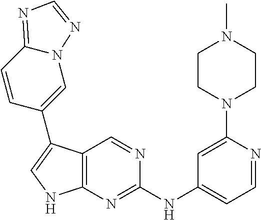 [SMILES: CN1CCN(c2cc(Nc3ncc4c(-c5ccc6ncnn6c5)c[nH]c4n3)ccn2)CC1] | 755 |
| 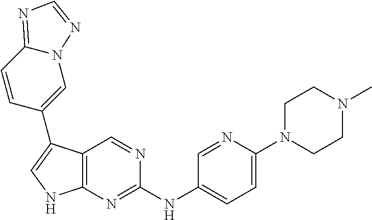 [SMILES: CN1CCN(c2ccc(Nc3ncc4c(-c5ccc6ncnn6c5)c[nH]c4n3)cn2)CC1] | 756 |
| 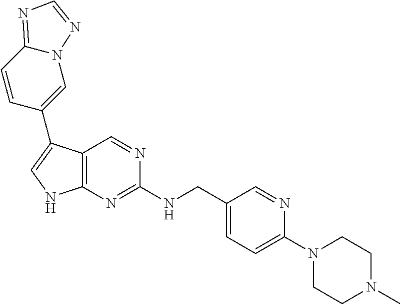 [SMILES: CN1CCN(c2ccc(CNc3ncc4c(-c5ccc6ncnn6c5)c[nH]c4n3)cn2)CC1] | 757 |
| 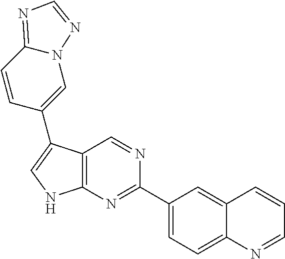 [SMILES: c1cnc2ccc(-c3ncc4c(-c5ccc6ncnn6c5)c[nH]c4n3)cc2c1] | 758 |
| 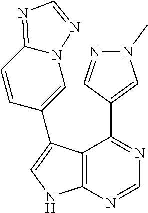 [SMILES: Cn1cc(-c2ncnc3[nH]cc(-c4ccc5ncnn5c4)c23)cn1] | 759 |
| 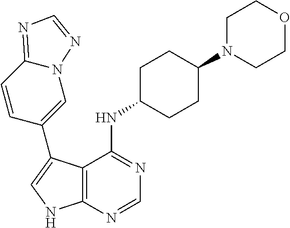 [SMILES: c1nc(N[C@H]2CC[C@H](N3CCOCC3)CC2)c2c(-c3ccc4ncnn4c3)c[nH]c2n1] | 760 |
| 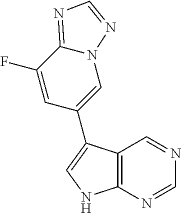 [SMILES: Fc1cc(-c2c[nH]c3ncncc23)cn2ncnc12] | 761 |
| 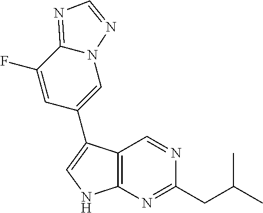 [SMILES: CC(C)Cc1ncc2c(-c3cc(F)c4ncnn4c3)c[nH]c2n1] | 762 |
| 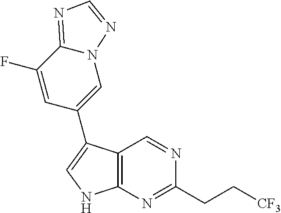 [SMILES: Fc1cc(-c2c[nH]c3nc(CCC(F)(F)F)ncc23)cn2ncnc12] | 763 |
| 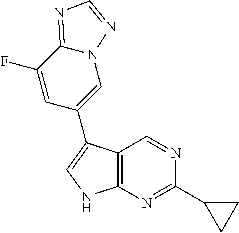 [SMILES: Fc1cc(-c2c[nH]c3nc(C4CC4)ncc23)cn2ncnc12] | 764 |
| 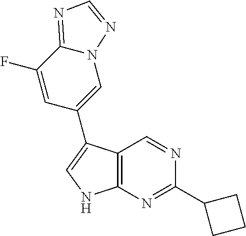 [SMILES: Fc1cc(-c2c[nH]c3nc(C4CCC4)ncc23)cn2ncnc12] | 765 |
| 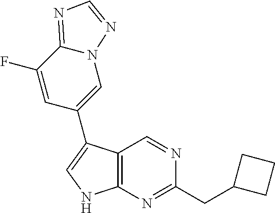 [SMILES: Fc1cc(-c2c[nH]c3nc(CC4CCC4)ncc23)cn2ncnc12] | 766 |
| 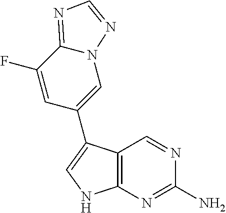 [SMILES: Nc1ncc2c(-c3cc(F)c4ncnn4c3)c[nH]c2n1] | 767 |
| 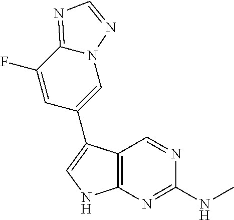 [SMILES: CNc1ncc2c(-c3cc(F)c4ncnn4c3)c[nH]c2n1] | 768 |
| 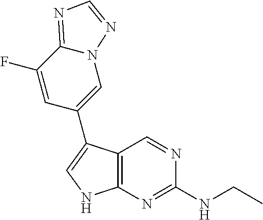 [SMILES: CCNc1ncc2c(-c3cc(F)c4ncnn4c3)c[nH]c2n1] | 769 |
| 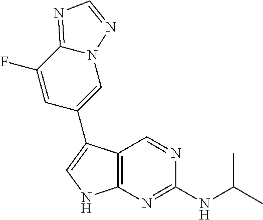 [SMILES: CC(C)Nc1ncc2c(-c3cc(F)c4ncnn4c3)c[nH]c2n1] | 770 |
| 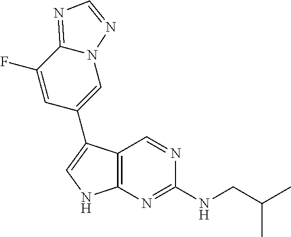 [SMILES: CC(C)CNc1ncc2c(-c3cc(F)c4ncnn4c3)c[nH]c2n1] | 771 |
| 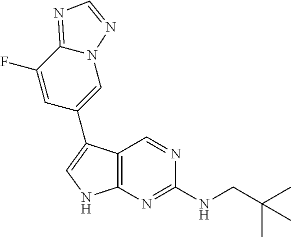 [SMILES: CC(C)(C)CNc1ncc2c(-c3cc(F)c4ncnn4c3)c[nH]c2n1] | 772 |
| 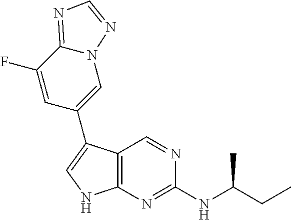 [SMILES: CC[C@H](C)Nc1ncc2c(-c3cc(F)c4ncnn4c3)c[nH]c2n1] | 773 |
| 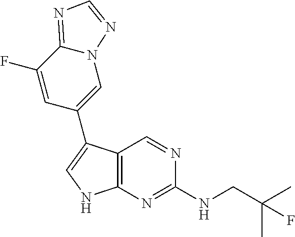 [SMILES: CC(C)(F)CNc1ncc2c(-c3cc(F)c4ncnn4c3)c[nH]c2n1] | 774 |
| 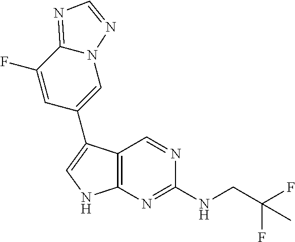 [SMILES: CC(F)(F)CNc1ncc2c(-c3cc(F)c4ncnn4c3)c[nH]c2n1] | 775 |
| 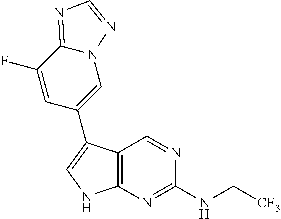 [SMILES: Fc1cc(-c2c[nH]c3nc(NCC(F)(F)F)ncc23)cn2ncnc12] | 776 |
| 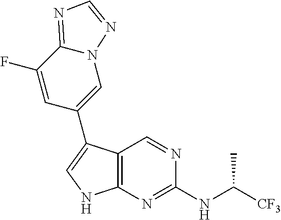 [SMILES: C[C@@H](Nc1ncc2c(-c3cc(F)c4ncnn4c3)c[nH]c2n1)C(F)(F)F] | 777 |
| 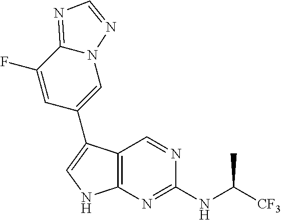 [SMILES: C[C@H](Nc1ncc2c(-c3cc(F)c4ncnn4c3)c[nH]c2n1)C(F)(F)F] | 778 |
| 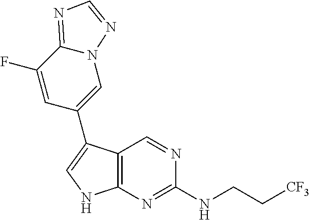 [SMILES: Fc1cc(-c2c[nH]c3nc(NCCC(F)(F)F)ncc23)cn2ncnc12] | 779 |
| 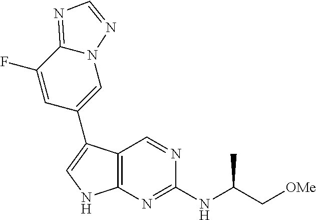 [SMILES: COC[C@H](C)Nc1ncc2c(-c3cc(F)c4ncnn4c3)c[nH]c2n1] | 780 |
| 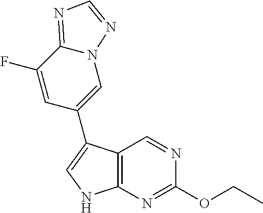 [SMILES: CCOc1ncc2c(-c3cc(F)c4ncnn4c3)c[nH]c2n1] | 781 |
| 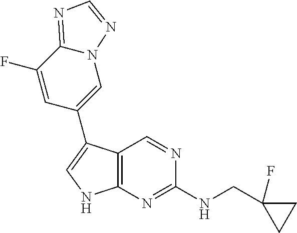 [SMILES: Fc1cc(-c2c[nH]c3nc(NCC4(F)CC4)ncc23)cn2ncnc12] | 782 |
| 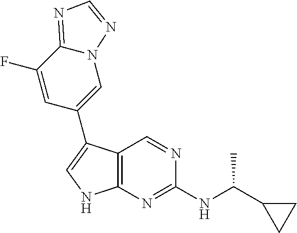 [SMILES: C[C@@H](Nc1ncc2c(-c3cc(F)c4ncnn4c3)c[nH]c2n1)C1CC1] | 783 |
| 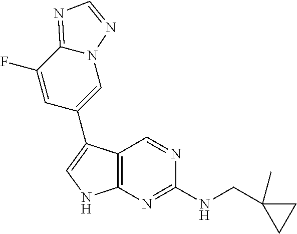 [SMILES: CC1(CNc2ncc3c(-c4cc(F)c5ncnn5c4)c[nH]c3n2)CC1] | 784 |
| 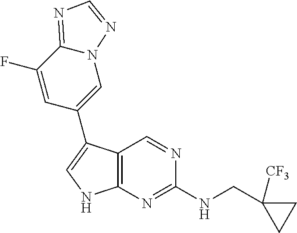 [SMILES: Fc1cc(-c2c[nH]c3nc(NCC4(C(F)(F)F)CC4)ncc23)cn2ncnc12] | 785 |
| 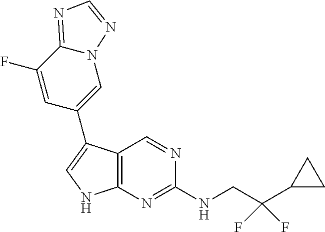 [SMILES: Fc1cc(-c2c[nH]c3nc(NCC(F)(F)C4CC4)ncc23)cn2ncnc12] | 786 |
| 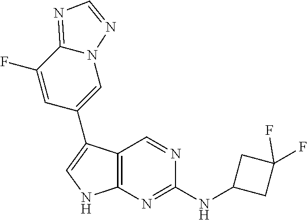 [SMILES: Fc1cc(-c2c[nH]c3nc(NC4CC(F)(F)C4)ncc23)cn2ncnc12] | 787 |
| 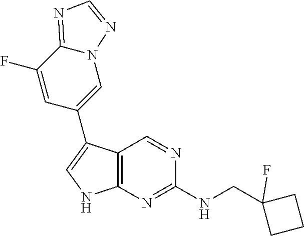 [SMILES: Fc1cc(-c2c[nH]c3nc(NCC4(F)CCC4)ncc23)cn2ncnc12] | 788 |
| 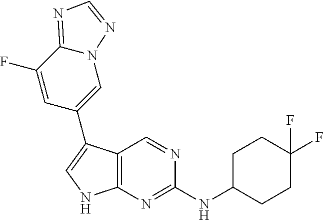 [SMILES: Fc1cc(-c2c[nH]c3nc(NC4CCC(F)(F)CC4)ncc23)cn2ncnc12] | 789 |
| 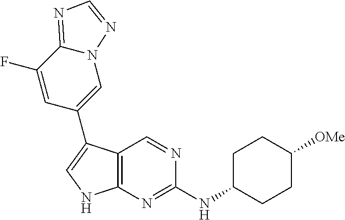 [SMILES: CO[C@H]1CC[C@@H](Nc2ncc3c(-c4cc(F)c5ncnn5c4)c[nH]c3n2)CC1] | 790 |
| 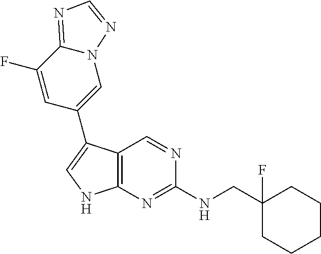 [SMILES: Fc1cc(-c2c[nH]c3nc(NCC4(F)CCCCC4)ncc23)cn2ncnc12] | 791 |
| 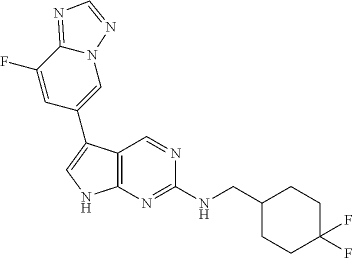 [SMILES: Fc1cc(-c2c[nH]c3nc(NCC4CCC(F)(F)CC4)ncc23)cn2ncnc12] | 792 |
| 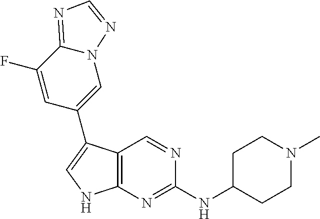 [SMILES: CN1CCC(Nc2ncc3c(-c4cc(F)c5ncnn5c4)c[nH]c3n2)CC1] | 793 |
| 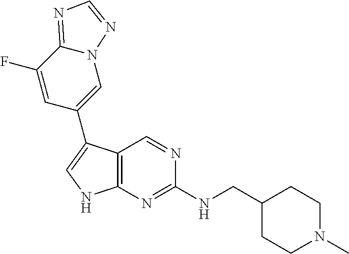 [SMILES: CN1CCC(CNc2ncc3c(-c4cc(F)c5ncnn5c4)c[nH]c3n2)CC1] | 794 |
| 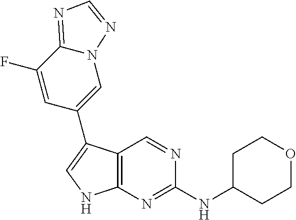 [SMILES: Fc1cc(-c2c[nH]c3nc(NC4CCOCC4)ncc23)cn2ncnc12] | 795 |
| 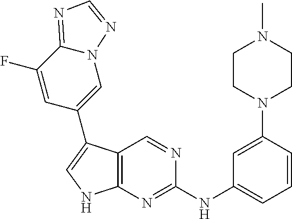 [SMILES: CN1CCN(c2cccc(Nc3ncc4c(-c5cc(F)c6ncnn6c5)c[nH]c4n3)c2)CC1] | 796 |
| 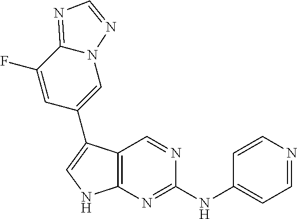 [SMILES: Fc1cc(-c2c[nH]c3nc(Nc4ccncc4)ncc23)cn2ncnc12] | 797 |
| 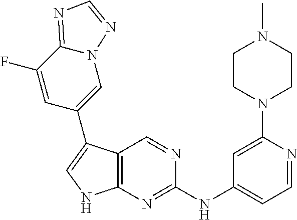 [SMILES: CN1CCN(c2cc(Nc3ncc4c(-c5cc(F)c6ncnn6c5)c[nH]c4n3)ccn2)CC1] | 798 |
| 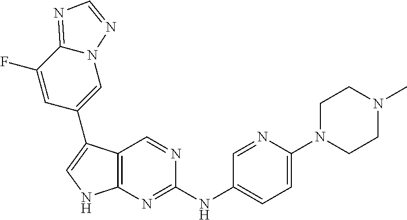 [SMILES: CN1CCN(c2ccc(Nc3ncc4c(-c5cc(F)c6ncnn6c5)c[nH]c4n3)cn2)CC1] | 799 |
| 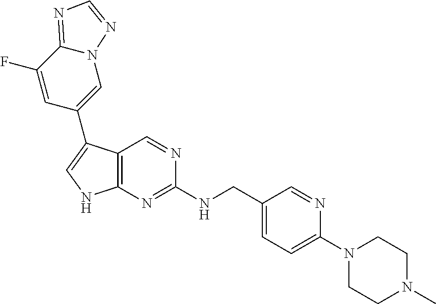 [SMILES: CN1CCN(c2ccc(CNc3ncc4c(-c5cc(F)c6ncnn6c5)c[nH]c4n3)cn2)CC1] | 800 |
| 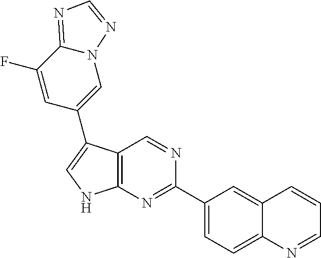 [SMILES: Fc1cc(-c2c[nH]c3nc(-c4ccc5ncccc5c4)ncc23)cn2ncnc12] | 801 |
| 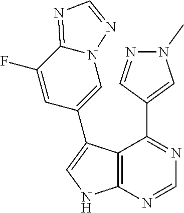 [SMILES: Cn1cc(-c2ncnc3[nH]cc(-c4cc(F)c5ncnn5c4)c23)cn1] | 802 |
| 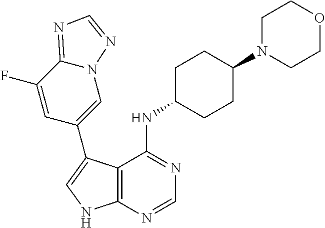 [SMILES: Fc1cc(-c2c[nH]c3ncnc(N[C@H]4CC[C@H](N5CCOCC5)CC4)c23)cn2ncnc12] | 803 |
| 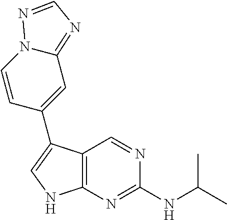 [SMILES: CC(C)Nc1ncc2c(-c3ccn4ncnc4c3)c[nH]c2n1] | 804 |
| 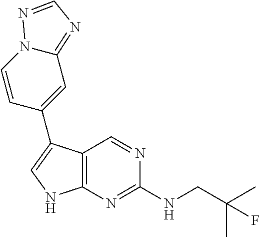 [SMILES: CC(C)(F)CNc1ncc2c(-c3ccn4ncnc4c3)c[nH]c2n1] | 805 |
| 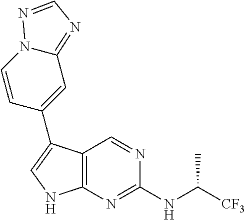 [SMILES: C[C@@H](Nc1ncc2c(-c3ccn4ncnc4c3)c[nH]c2n1)C(F)(F)F] | 806 |
| 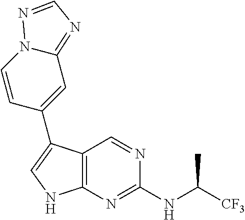 [SMILES: C[C@H](Nc1ncc2c(-c3ccn4ncnc4c3)c[nH]c2n1)C(F)(F)F] | 807 |
| 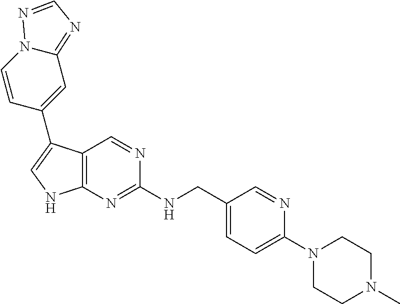 [SMILES: CN1CCN(c2ccc(CNc3ncc4c(-c5ccn6ncnc6c5)c[nH]c4n3)cn2)CC1] | 808 |
| 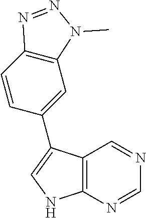 [SMILES: Cn1nnc2ccc(-c3c[nH]c4ncncc34)cc21] | 809 |
| 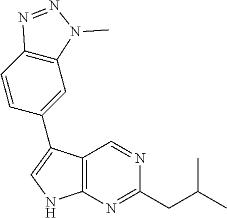 [SMILES: CC(C)Cc1ncc2c(-c3ccc4nnn(C)c4c3)c[nH]c2n1] | 810 |
| 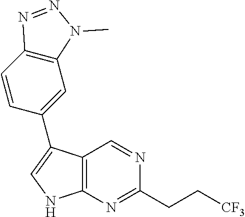 [SMILES: Cn1nnc2ccc(-c3c[nH]c4nc(CCC(F)(F)F)ncc34)cc21] | 811 |
| 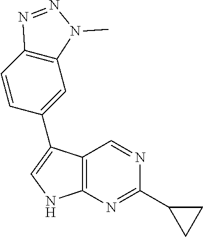 [SMILES: Cn1nnc2ccc(-c3c[nH]c4nc(C5CC5)ncc34)cc21] | 812 |
| 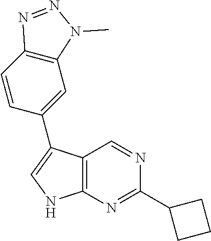 [SMILES: Cn1nnc2ccc(-c3c[nH]c4nc(C5CCC5)ncc34)cc21] | 813 |
| 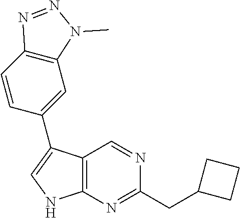 [SMILES: Cn1nnc2ccc(-c3c[nH]c4nc(CC5CCC5)ncc34)cc21] | 814 |
| 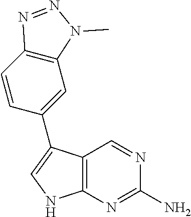 [SMILES: Cn1nnc2ccc(-c3c[nH]c4nc(N)ncc34)cc21] | 815 |
| 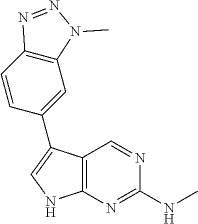 [SMILES: CNc1ncc2c(-c3ccc4nnn(C)c4c3)c[nH]c2n1] | 816 |
| 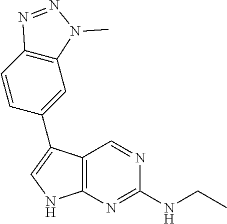 [SMILES: CCNc1ncc2c(-c3ccc4nnn(C)c4c3)c[nH]c2n1] | 817 |
| 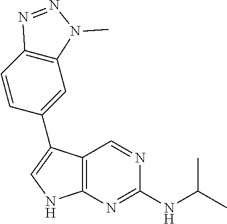 [SMILES: CC(C)Nc1ncc2c(-c3ccc4nnn(C)c4c3)c[nH]c2n1] | 818 |
| 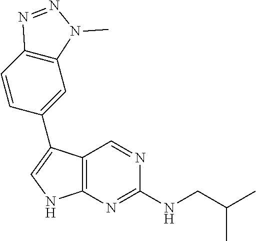 [SMILES: CC(C)CNc1ncc2c(-c3ccc4nnn(C)c4c3)c[nH]c2n1] | 819 |
| 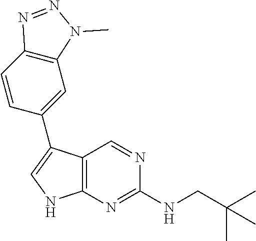 [SMILES: Cn1nnc2ccc(-c3c[nH]c4nc(NCC(C)(C)C)ncc34)cc21] | 820 |
| 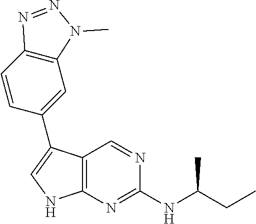 [SMILES: CC[C@H](C)Nc1ncc2c(-c3ccc4nnn(C)c4c3)c[nH]c2n1] | 821 |
| 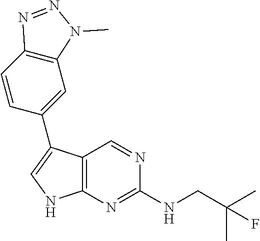 [SMILES: Cn1nnc2ccc(-c3c[nH]c4nc(NCC(C)(C)F)ncc34)cc21] | 822 |
| 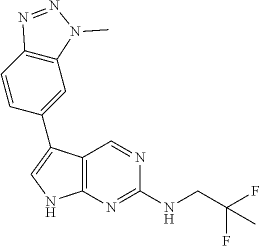 [SMILES: Cn1nnc2ccc(-c3c[nH]c4nc(NCC(C)(F)F)ncc34)cc21] | 823 |
| 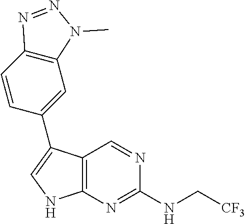 [SMILES: Cn1nnc2ccc(-c3c[nH]c4nc(NCC(F)(F)F)ncc34)cc21] | 824 |
| 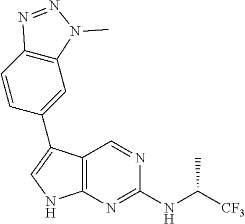 [SMILES: C[C@@H](Nc1ncc2c(-c3ccc4nnn(C)c4c3)c[nH]c2n1)C(F)(F)F] | 825 |
| 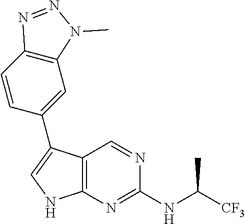 [SMILES: C[C@H](Nc1ncc2c(-c3ccc4nnn(C)c4c3)c[nH]c2n1)C(F)(F)F] | 826 |
| 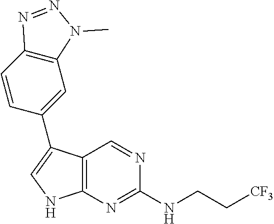 [SMILES: Cn1nnc2ccc(-c3c[nH]c4nc(NCCC(F)(F)F)ncc34)cc21] | 827 |
| 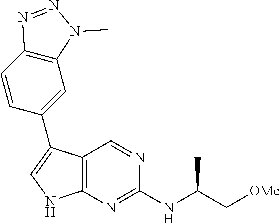 [SMILES: COC[C@H](C)Nc1ncc2c(-c3ccc4nnn(C)c4c3)c[nH]c2n1] | 828 |
| 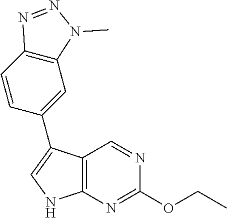 [SMILES: CCOc1ncc2c(-c3ccc4nnn(C)c4c3)c[nH]c2n1] | 829 |
| 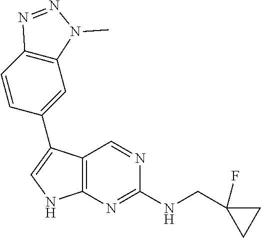 [SMILES: Cn1nnc2ccc(-c3c[nH]c4nc(NCC5(F)CC5)ncc34)cc21] | 830 |
| 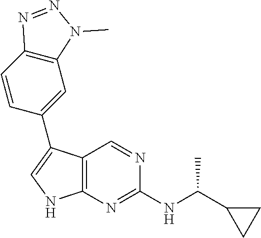 [SMILES: C[C@@H](Nc1ncc2c(-c3ccc4nnn(C)c4c3)c[nH]c2n1)C1CC1] | 831 |
| 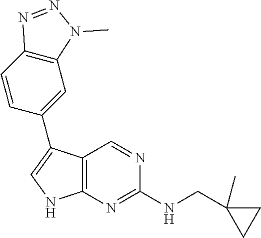 [SMILES: Cn1nnc2ccc(-c3c[nH]c4nc(NCC5(C)CC5)ncc34)cc21] | 832 |
| 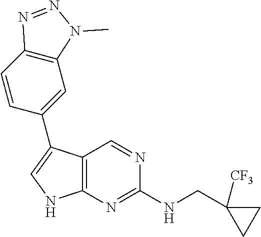 [SMILES: Cn1nnc2ccc(-c3c[nH]c4nc(NCC5(C(F)(F)F)CC5)ncc34)cc21] | 833 |
| 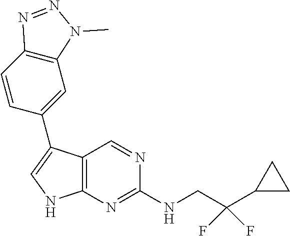 [SMILES: Cn1nnc2ccc(-c3c[nH]c4nc(NCC(F)(F)C5CC5)ncc34)cc21] | 834 |
| 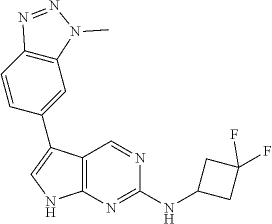 [SMILES: Cn1nnc2ccc(-c3c[nH]c4nc(NC5CC(F)(F)C5)ncc34)cc21] | 835 |
| 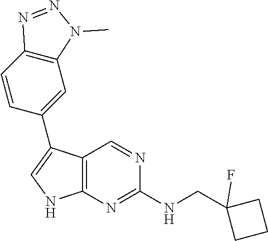 [SMILES: Cn1nnc2ccc(-c3c[nH]c4nc(NCC5(F)CCC5)ncc34)cc21] | 836 |
| 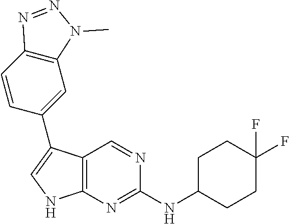 [SMILES: Cn1nnc2ccc(-c3c[nH]c4nc(NC5CCC(F)(F)CC5)ncc34)cc21] | 837 |
| 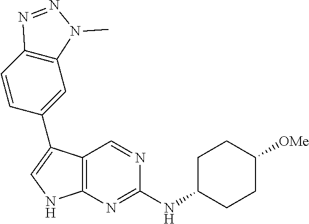 [SMILES: CO[C@H]1CC[C@@H](Nc2ncc3c(-c4ccc5nnn(C)c5c4)c[nH]c3n2)CC1] | 838 |
| 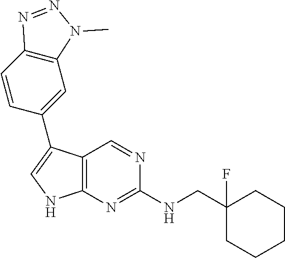 [SMILES: Cn1nnc2ccc(-c3c[nH]c4nc(NCC5(F)CCCCC5)ncc34)cc21] | 839 |
| 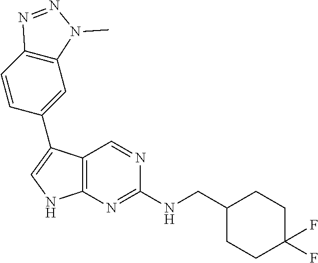 [SMILES: Cn1nnc2ccc(-c3c[nH]c4nc(NCC5CCC(F)(F)CC5)ncc34)cc21] | 840 |
| 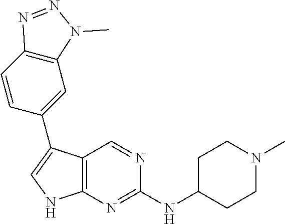 [SMILES: CN1CCC(Nc2ncc3c(-c4ccc5nnn(C)c5c4)c[nH]c3n2)CC1] | 841 |
| 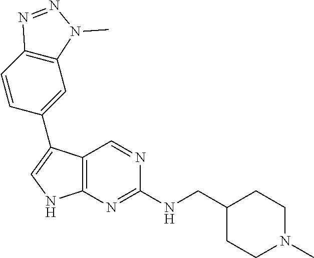 [SMILES: CN1CCC(CNc2ncc3c(-c4ccc5nnn(C)c5c4)c[nH]c3n2)CC1] | 842 |
| 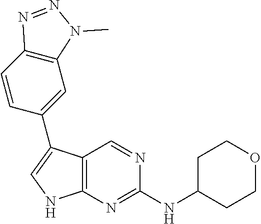 [SMILES: Cn1nnc2ccc(-c3c[nH]c4nc(NC5CCOCC5)ncc34)cc21] | 843 |
| 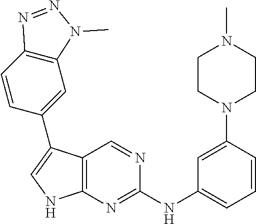 [SMILES: CN1CCN(c2cccc(Nc3ncc4c(-c5ccc6nnn(C)c6c5)c[nH]c4n3)c2)CC1] | 844 |
| 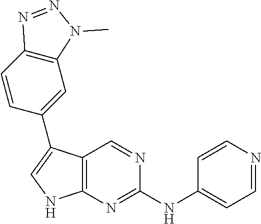 [SMILES: Cn1nnc2ccc(-c3c[nH]c4nc(Nc5ccncc5)ncc34)cc21] | 845 |
| 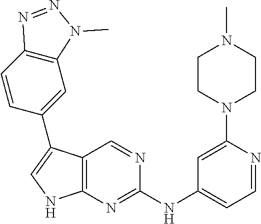 [SMILES: CN1CCN(c2cc(Nc3ncc4c(-c5ccc6nnn(C)c6c5)c[nH]c4n3)ccn2)CC1] | 846 |
| 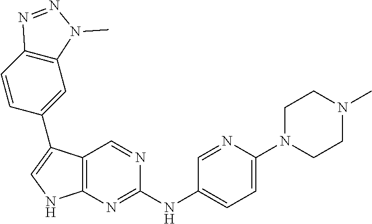 [SMILES: CN1CCN(c2ccc(Nc3ncc4c(-c5ccc6nnn(C)c6c5)c[nH]c4n3)cn2)CC1] | 847 |
| 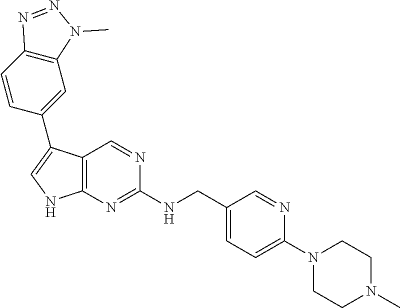 [SMILES: CN1CCN(c2ccc(CNc3ncc4c(-c5ccc6nnn(C)c6c5)c[nH]c4n3)cn2)CC1] | 848 |
| 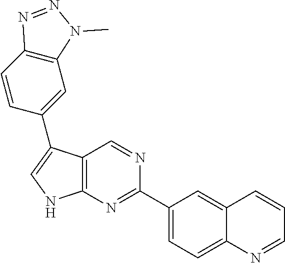 [SMILES: Cn1nnc2ccc(-c3c[nH]c4nc(-c5ccc6ncccc6c5)ncc34)cc21] | 849 |
| 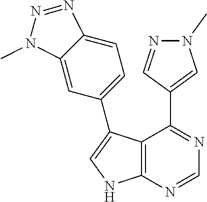 | 850 |
| 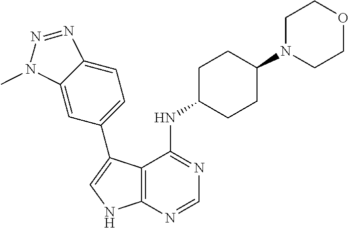 [SMILES: Cn1nnc2ccc(-c3c[nH]c4ncnc(N[C@H]5CC[C@H](N6CCOCC6)CC5)c34)cc21] | 851 |
| 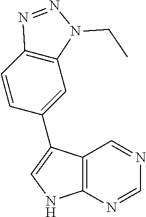 [SMILES: CCn1nnc2ccc(-c3c[nH]c4ncncc34)cc21] | 852 |
| 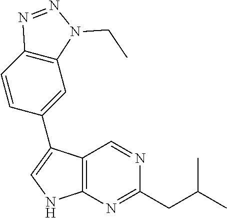 [SMILES: CCn1nnc2ccc(-c3c[nH]c4nc(CC(C)C)ncc34)cc21] | 853 |
| 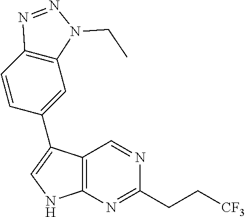 [SMILES: CCn1nnc2ccc(-c3c[nH]c4nc(CCC(F)(F)F)ncc34)cc21] | 854 |
| 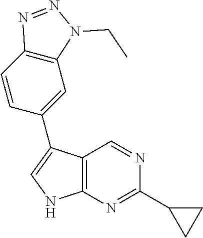 [SMILES: CCn1nnc2ccc(-c3c[nH]c4nc(C5CC5)ncc34)cc21] | 855 |
| 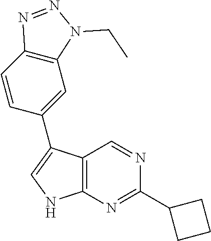 [SMILES: CCn1nnc2ccc(-c3c[nH]c4nc(C5CCC5)ncc34)cc21] | 856 |
| 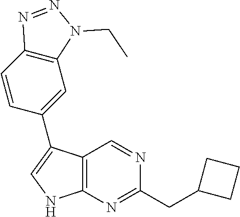 [SMILES: CCn1nnc2ccc(-c3c[nH]c4nc(CC5CCC5)ncc34)cc21] | 857 |
| 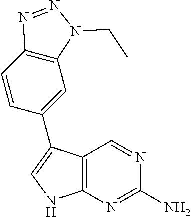 [SMILES: CCn1nnc2ccc(-c3c[nH]c4nc(N)ncc34)cc21] | 858 |
| 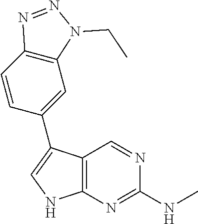 [SMILES: CCn1nnc2ccc(-c3c[nH]c4nc(NC)ncc34)cc21] | 859 |
| 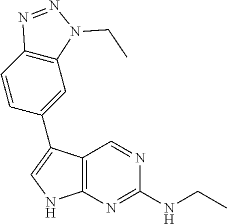 [SMILES: CCNc1ncc2c(-c3ccc4nnn(CC)c4c3)c[nH]c2n1] | 860 |
| 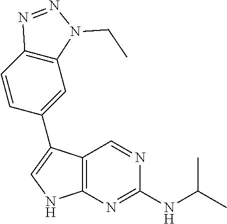 [SMILES: CCn1nnc2ccc(-c3c[nH]c4nc(NC(C)C)ncc34)cc21] | 861 |
| 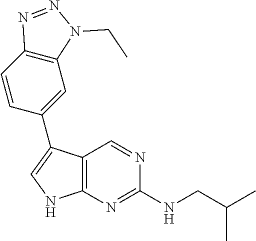 [SMILES: CCn1nnc2ccc(-c3c[nH]c4nc(NCC(C)C)ncc34)cc21] | 862 |
| 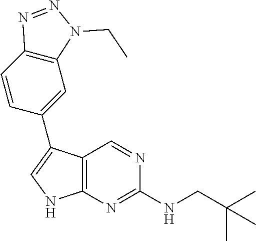 [SMILES: CCn1nnc2ccc(-c3c[nH]c4nc(NCC(C)(C)C)ncc34)cc21] | 863 |
| 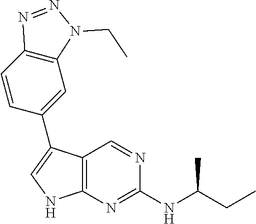 [SMILES: CC[C@H](C)Nc1ncc2c(-c3ccc4nnn(CC)c4c3)c[nH]c2n1] | 864 |
| 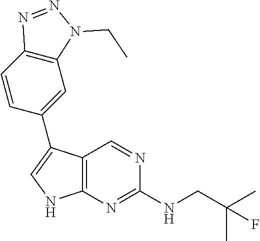 [SMILES: CCn1nnc2ccc(-c3c[nH]c4nc(NCC(C)(C)F)ncc34)cc21] | 865 |
| 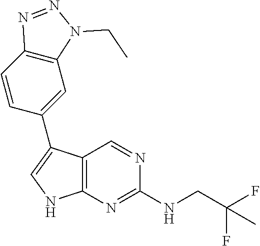 [SMILES: CCn1nnc2ccc(-c3c[nH]c4nc(NCC(C)(F)F)ncc34)cc21] | 866 |
| 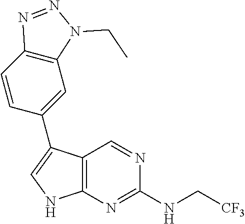 [SMILES: CCn1nnc2ccc(-c3c[nH]c4nc(NCC(F)(F)F)ncc34)cc21] | 867 |
| 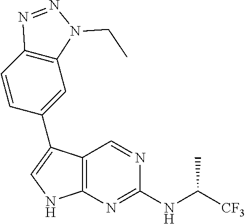 [SMILES: CCn1nnc2ccc(-c3c[nH]c4nc(N[C@H](C)C(F)(F)F)ncc34)cc21] | 868 |
| 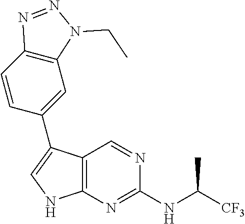 [SMILES: CCn1nnc2ccc(-c3c[nH]c4nc(N[C@@H](C)C(F)(F)F)ncc34)cc21] | 869 |
| 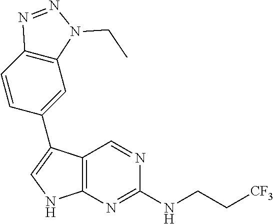 [SMILES: CCn1nnc2ccc(-c3c[nH]c4nc(NCCC(F)(F)F)ncc34)cc21] | 870 |
| 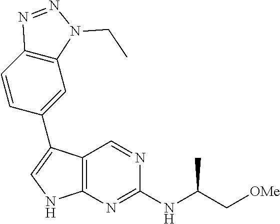 [SMILES: CCn1nnc2ccc(-c3c[nH]c4nc(N[C@@H](C)COC)ncc34)cc21] | 871 |
| 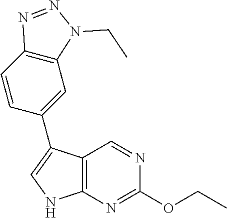 [SMILES: CCOc1ncc2c(-c3ccc4nnn(CC)c4c3)c[nH]c2n1] | 872 |
| 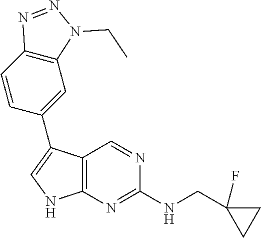 [SMILES: CCn1nnc2ccc(-c3c[nH]c4nc(NCC5(F)CC5)ncc34)cc21] | 873 |
| 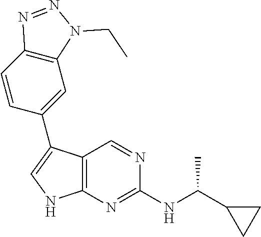 [SMILES: CCn1nnc2ccc(-c3c[nH]c4nc(N[C@H](C)C5CC5)ncc34)cc21] | 874 |
| 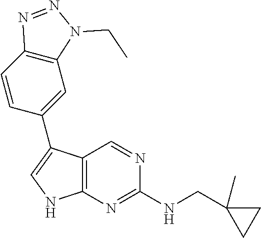 [SMILES: CCn1nnc2ccc(-c3c[nH]c4nc(NCC5(C)CC5)ncc34)cc21] | 875 |
| 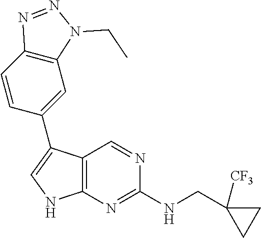 [SMILES: CCn1nnc2ccc(-c3c[nH]c4nc(NCC5(C(F)(F)F)CC5)ncc34)cc21] | 876 |
| 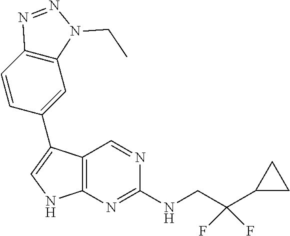 [SMILES: CCn1nnc2ccc(-c3c[nH]c4nc(NCC(F)(F)C5CC5)ncc34)cc21] | 877 |
| 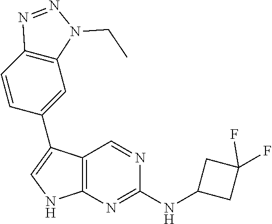 [SMILES: CCn1nnc2ccc(-c3c[nH]c4nc(NC5CC(F)(F)C5)ncc34)cc21] | 878 |
| 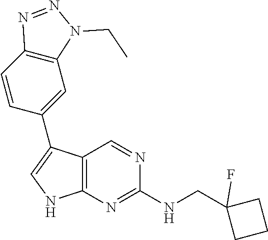 [SMILES: CCn1nnc2ccc(-c3c[nH]c4nc(NCC5(F)CCC5)ncc34)cc21] | 879 |
| 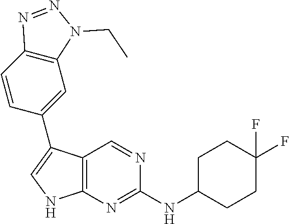 [SMILES: CCn1nnc2ccc(-c3c[nH]c4nc(NC5CCC(F)(F)CC5)ncc34)cc21] | 880 |
| 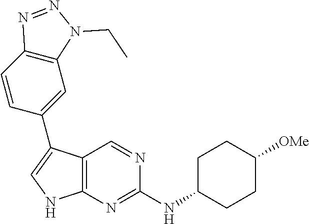 [SMILES: CCn1nnc2ccc(-c3c[nH]c4nc(N[C@H]5CC[C@@H](OC)CC5)ncc34)cc21] | 881 |
| 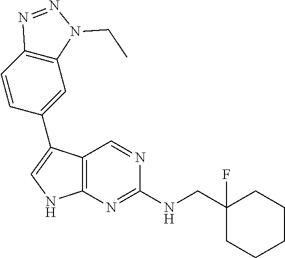 [SMILES: CCn1nnc2ccc(-c3c[nH]c4nc(NCC5(F)CCCCC5)ncc34)cc21] | 882 |
| 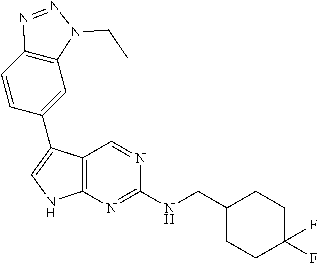 [SMILES: CCn1nnc2ccc(-c3c[nH]c4nc(NCC5CCC(F)(F)CC5)ncc34)cc21] | 883 |
| 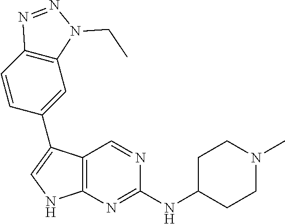 [SMILES: CCn1nnc2ccc(-c3c[nH]c4nc(NC5CCN(C)CC5)ncc34)cc21] | 884 |
| 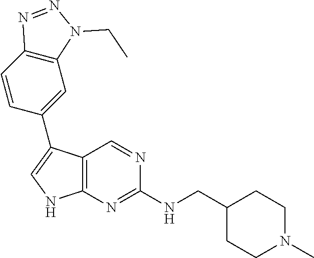 [SMILES: CCn1nnc2ccc(-c3c[nH]c4nc(NCC5CCN(C)CC5)ncc34)cc21] | 885 |
| 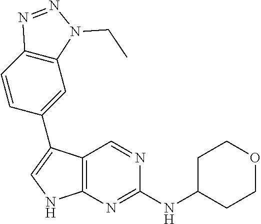 [SMILES: CCn1nnc2ccc(-c3c[nH]c4nc(NC5CCOCC5)ncc34)cc21] | 886 |
| 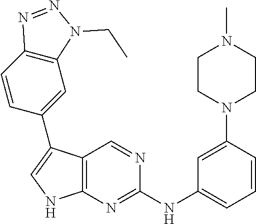 [SMILES: CCn1nnc2ccc(-c3c[nH]c4nc(Nc5cccc(N6CCN(C)CC6)c5)ncc34)cc21] | 887 |
| 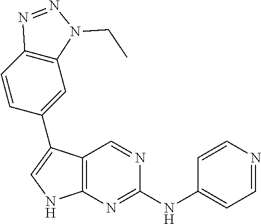 [SMILES: CCn1nnc2ccc(-c3c[nH]c4nc(Nc5ccncc5)ncc34)cc21] | 888 |
| 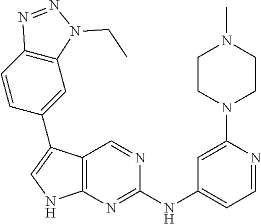 [SMILES: CCn1nnc2ccc(-c3c[nH]c4nc(Nc5ccnc(N6CCN(C)CC6)c5)ncc34)cc21] | 889 |
| 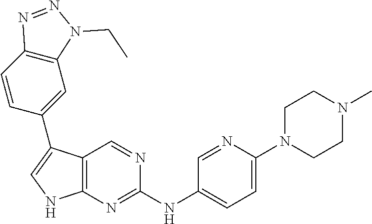 [SMILES: CCn1nnc2ccc(-c3c[nH]c4nc(Nc5ccc(N6CCN(C)CC6)nc5)ncc34)cc21] | 890 |
| 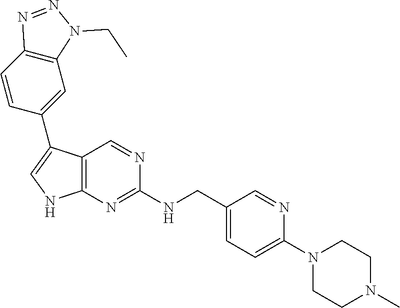 [SMILES: CCn1nnc2ccc(-c3c[nH]c4nc(NCc5ccc(N6CCN(C)CC6)nc5)ncc34)cc21] | 891 |
| 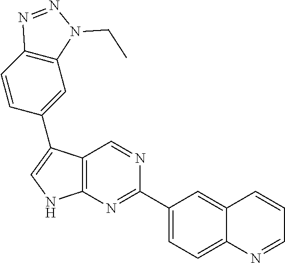 [SMILES: CCn1nnc2ccc(-c3c[nH]c4nc(-c5ccc6ncccc6c5)ncc34)cc21] | 892 |
| 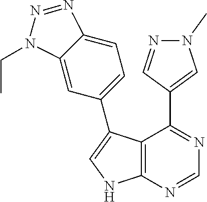 [SMILES: CCn1nnc2ccc(-c3c[nH]c4ncnc(-c5cnn(C)c5)c34)cc21] | 893 |
| 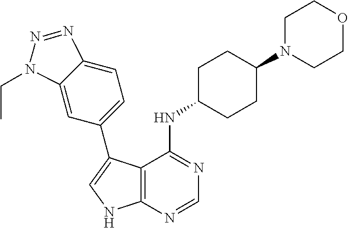 [SMILES: CCn1nnc2ccc(-c3c[nH]c4ncnc(N[C@H]5CC[C@H](N6CCOCC6)CC5)c34)cc21] | 894 |
| 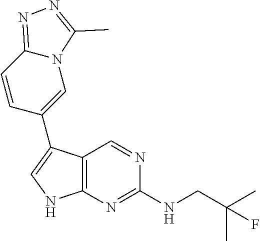 [SMILES: Cc1nnc2ccc(-c3c[nH]c4nc(NCC(C)(C)F)ncc34)cn12] | 895 |
| 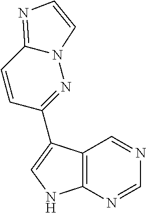 [SMILES: c1ncc2c(-c3ccc4nccn4n3)c[nH]c2n1] | 896 |
| 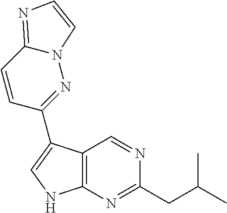 [SMILES: CC(C)Cc1ncc2c(-c3ccc4nccn4n3)c[nH]c2n1] | 897 |
| 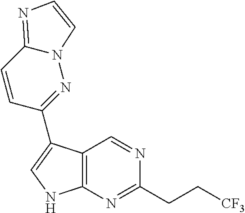 [SMILES: FC(F)(F)CCc1ncc2c(-c3ccc4nccn4n3)c[nH]c2n1] | 898 |
| 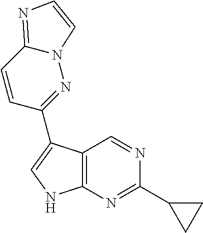 [SMILES: c1cn2nc(-c3c[nH]c4nc(C5CC5)ncc34)ccc2n1] | 899 |
| 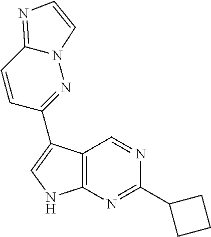 [SMILES: c1cn2nc(-c3c[nH]c4nc(C5CCC5)ncc34)ccc2n1] | 900 |
| 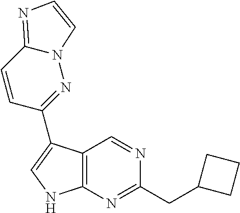 [SMILES: c1cn2nc(-c3c[nH]c4nc(CC5CCC5)ncc34)ccc2n1] | 901 |
| 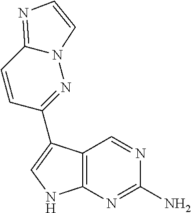 [SMILES: Nc1ncc2c(-c3ccc4nccn4n3)c[nH]c2n1] | 902 |
| 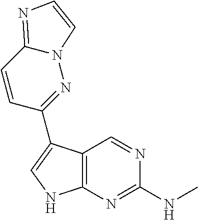 [SMILES: CNc1ncc2c(-c3ccc4nccn4n3)c[nH]c2n1] | 903 |
| 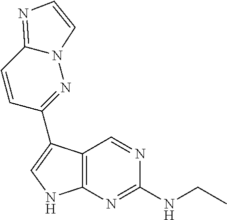 [SMILES: CCNc1ncc2c(-c3ccc4nccn4n3)c[nH]c2n1] | 904 |
| 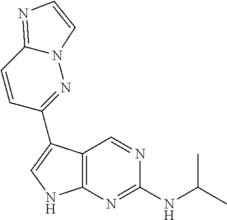 [SMILES: CC(C)Nc1ncc2c(-c3ccc4nccn4n3)c[nH]c2n1] | 905 |
| 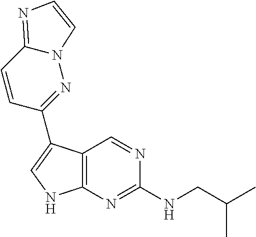 [SMILES: CC(C)CNc1ncc2c(-c3ccc4nccn4n3)c[nH]c2n1] | 906 |
| 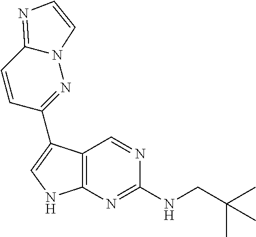 [SMILES: CC(C)(C)CNc1ncc2c(-c3ccc4nccn4n3)c[nH]c2n1] | 907 |
| 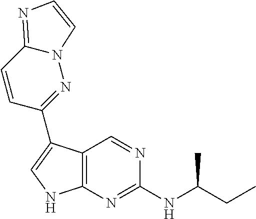 [SMILES: CC[C@H](C)Nc1ncc2c(-c3ccc4nccn4n3)c[nH]c2n1] | 908 |
| 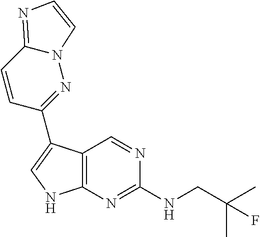 [SMILES: CC(C)(F)CNc1ncc2c(-c3ccc4nccn4n3)c[nH]c2n1] | 909 |
| 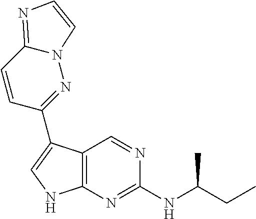 [SMILES: CC[C@H](C)Nc1ncc2c(-c3ccc4nccn4n3)c[nH]c2n1] | 910 |
| 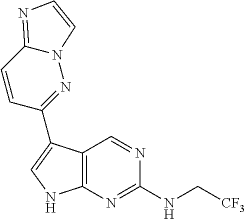 [SMILES: FC(F)(F)CNc1ncc2c(-c3ccc4nccn4n3)c[nH]c2n1] | 911 |
| 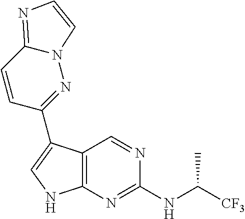 [SMILES: C[C@@H](Nc1ncc2c(-c3ccc4nccn4n3)c[nH]c2n1)C(F)(F)F] | 912 |
| 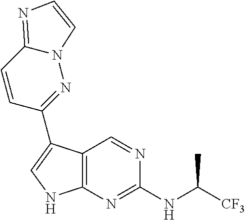 [SMILES: C[C@H](Nc1ncc2c(-c3ccc4nccn4n3)c[nH]c2n1)C(F)(F)F] | 913 |
| 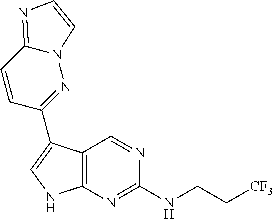 [SMILES: FC(F)(F)CCNc1ncc2c(-c3ccc4nccn4n3)c[nH]c2n1] | 914 |
| 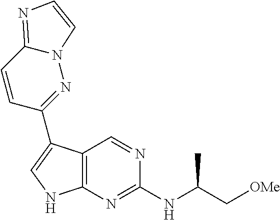 [SMILES: COC[C@H](C)Nc1ncc2c(-c3ccc4nccn4n3)c[nH]c2n1] | 915 |
| 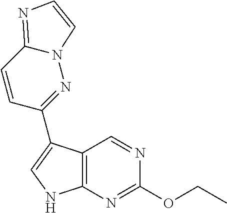 [SMILES: CCOc1ncc2c(-c3ccc4nccn4n3)c[nH]c2n1] | 916 |
| 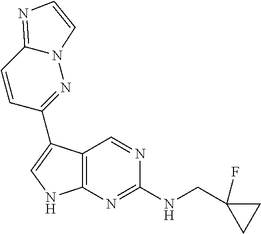 [SMILES: FC1(CNc2ncc3c(-c4ccc5nccn5n4)c[nH]c3n2)CC1] | 917 |
| 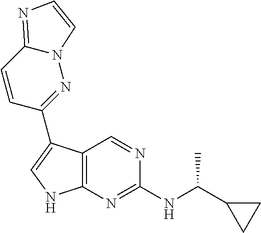 [SMILES: C[C@@H](Nc1ncc2c(-c3ccc4nccn4n3)c[nH]c2n1)C1CC1] | 918 |
| 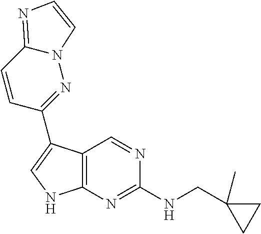 [SMILES: CC1(CNc2ncc3c(-c4ccc5nccn5n4)c[nH]c3n2)CC1] | 919 |
| 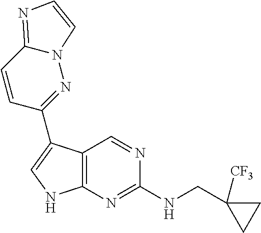 [SMILES: FC(F)(F)C1(CNc2ncc3c(-c4ccc5nccn5n4)c[nH]c3n2)CC1] | 920 |
| 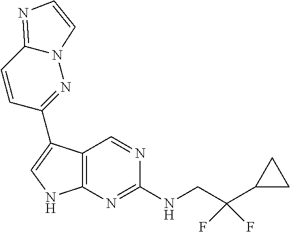 [SMILES: FC(F)(CNc1ncc2c(-c3ccc4nccn4n3)c[nH]c2n1)C1CC1] | 921 |
| 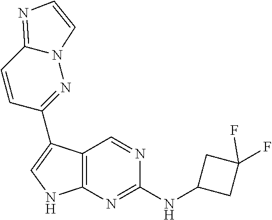 [SMILES: FC1(F)CC(Nc2ncc3c(-c4ccc5nccn5n4)c[nH]c3n2)C1] | 922 |
| 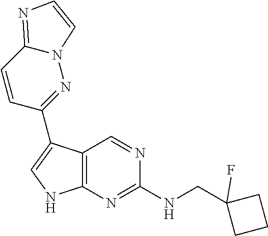 [SMILES: FC1(CNc2ncc3c(-c4ccc5nccn5n4)c[nH]c3n2)CCC1] | 923 |
| 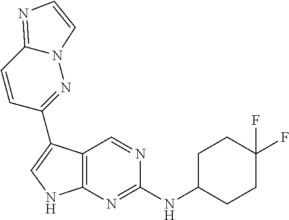 [SMILES: FC1(F)CCC(Nc2ncc3c(-c4ccc5nccn5n4)c[nH]c3n2)CC1] | 924 |
| 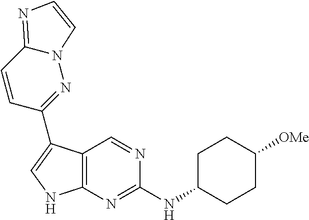 [SMILES: CO[C@H]1CC[C@@H](Nc2ncc3c(-c4ccc5nccn5n4)c[nH]c3n2)CC1] | 925 |
| 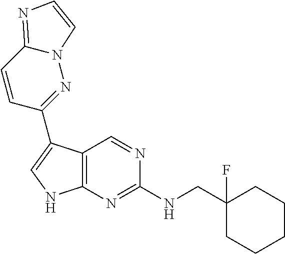 [SMILES: FC1(CNc2ncc3c(-c4ccc5nccn5n4)c[nH]c3n2)CCCCC1] | 926 |
| 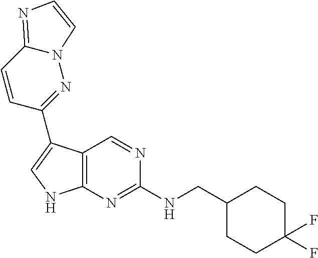 [SMILES: FC1(F)CCC(CNc2ncc3c(-c4ccc5nccn5n4)c[nH]c3n2)CC1] | 927 |
| 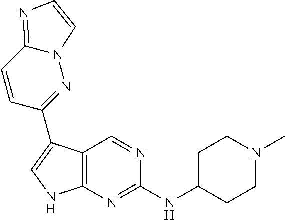 [SMILES: CN1CCC(Nc2ncc3c(-c4ccc5nccn5n4)c[nH]c3n2)CC1] | 928 |
| 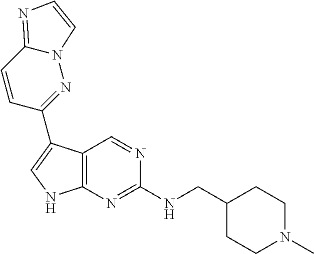 [SMILES: CN1CCC(CNc2ncc3c(-c4ccc5nccn5n4)c[nH]c3n2)CC1] | 929 |
| 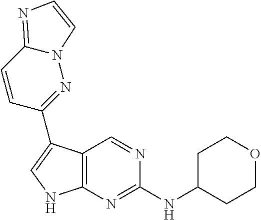 [SMILES: c1cn2nc(-c3c[nH]c4nc(NC5CCOCC5)ncc34)ccc2n1] | 930 |
| 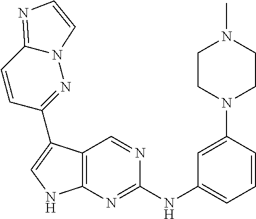 [SMILES: CN1CCN(c2cccc(Nc3ncc4c(-c5ccc6nccn6n5)c[nH]c4n3)c2)CC1] | 931 |
| 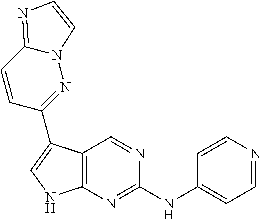 [SMILES: c1cc(Nc2ncc3c(-c4ccc5nccn5n4)c[nH]c3n2)ccn1] | 932 |
| 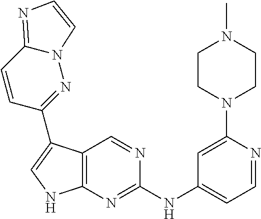 [SMILES: CN1CCN(c2cc(Nc3ncc4c(-c5ccc6nccn6n5)c[nH]c4n3)ccn2)CC1] | 933 |
| 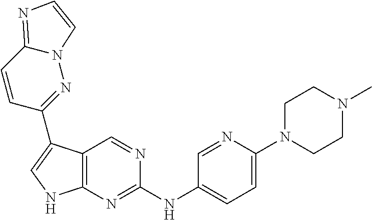 [SMILES: CN1CCN(c2ccc(Nc3ncc4c(-c5ccc6nccn6n5)c[nH]c4n3)cn2)CC1] | 934 |
| 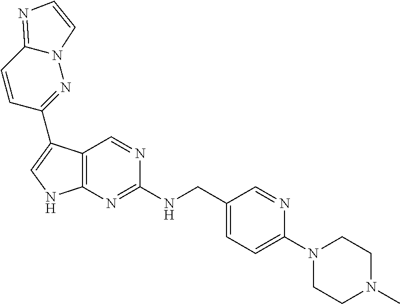 [SMILES: CN1CCN(c2ccc(CNc3ncc4c(-c5ccc6nccn6n5)c[nH]c4n3)cn2)CC1] | 935 |
| 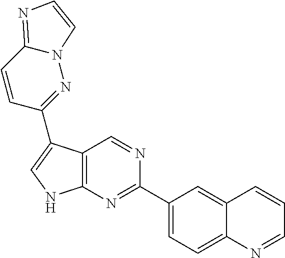 [SMILES: c1cnc2ccc(-c3ncc4c(-c5ccc6nccn6n5)c[nH]c4n3)cc2c1] | 936 |
| 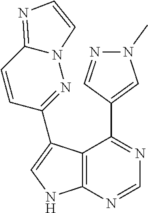 [SMILES: Cn1cc(-c2ncnc3[nH]cc(-c4ccc5nccn5n4)c23)cn1] | 937 |
| 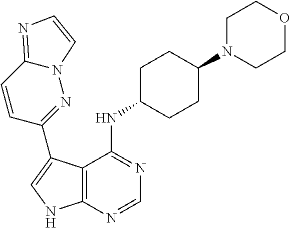 [SMILES: c1nc(N[C@H]2CC[C@H](N3CCOCC3)CC2)c2c(-c3ccc4nccn4n3)c[nH]c2n1] | 938 |
| 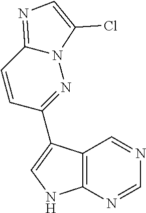 [SMILES: Clc1cnc2ccc(-c3c[nH]c4ncncc34)nn12] | 939 |
| 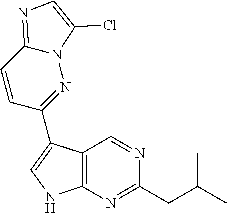 [SMILES: CC(C)Cc1ncc2c(-c3ccc4ncc(Cl)n4n3)c[nH]c2n1] | 940 |
| 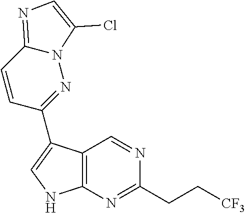 [SMILES: FC(F)(F)CCc1ncc2c(-c3ccc4ncc(Cl)n4n3)c[nH]c2n1] | 941 |
| 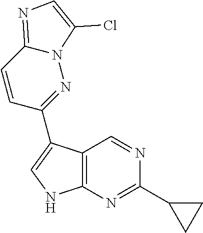 [SMILES: Clc1cnc2ccc(-c3c[nH]c4nc(C5CC5)ncc34)nn12] | 942 |
| 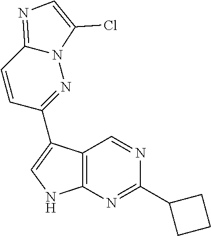 [SMILES: Clc1cnc2ccc(-c3c[nH]c4nc(C5CCC5)ncc34)nn12] | 943 |
| 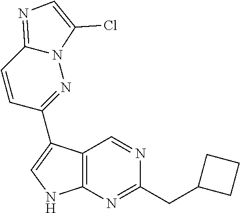 [SMILES: Clc1cnc2ccc(-c3c[nH]c4nc(CC5CCC5)ncc34)nn12] | 944 |
| 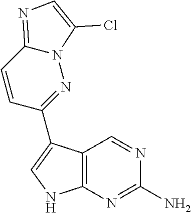 [SMILES: Nc1ncc2c(-c3ccc4ncc(Cl)n4n3)c[nH]c2n1] | 945 |
| 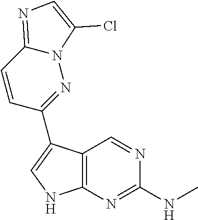 [SMILES: CNc1ncc2c(-c3ccc4ncc(Cl)n4n3)c[nH]c2n1] | 946 |
| 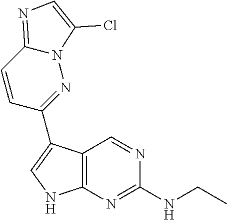 [SMILES: CCNc1ncc2c(-c3ccc4ncc(Cl)n4n3)c[nH]c2n1] | 947 |
| 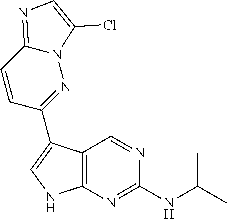 [SMILES: CC(C)Nc1ncc2c(-c3ccc4ncc(Cl)n4n3)c[nH]c2n1] | 948 |
| 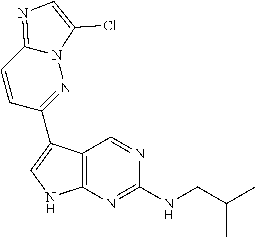 [SMILES: CC(C)CNc1ncc2c(-c3ccc4ncc(Cl)n4n3)c[nH]c2n1] | 949 |
| 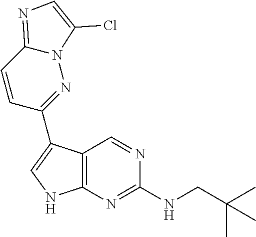 [SMILES: CC(C)(C)CNc1ncc2c(-c3ccc4ncc(Cl)n4n3)c[nH]c2n1] | 950 |
| 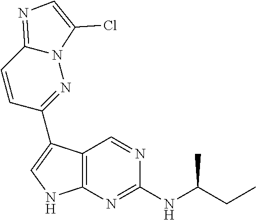 [SMILES: CC[C@H](C)Nc1ncc2c(-c3ccc4ncc(Cl)n4n3)c[nH]c2n1] | 951 |
| 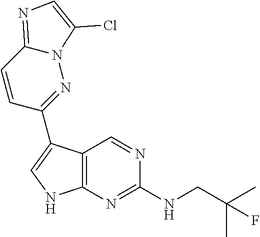 [SMILES: CC(C)(F)CNc1ncc2c(-c3ccc4ncc(Cl)n4n3)c[nH]c2n1] | 952 |
| 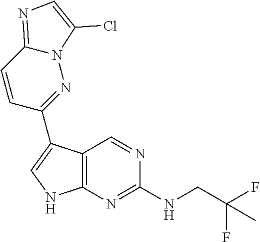 [SMILES: CC(F)(F)CNc1ncc2c(-c3ccc4ncc(Cl)n4n3)c[nH]c2n1] | 953 |
| 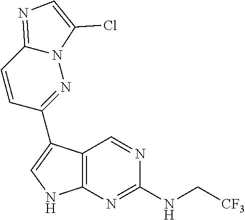 [SMILES: FC(F)(F)CNc1ncc2c(-c3ccc4ncc(Cl)n4n3)c[nH]c2n1] | 954 |
| 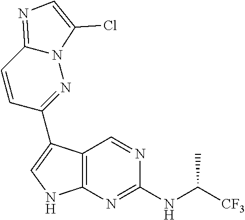 [SMILES: C[C@@H](Nc1ncc2c(-c3ccc4ncc(Cl)n4n3)c[nH]c2n1)C(F)(F)F] | 955 |
| 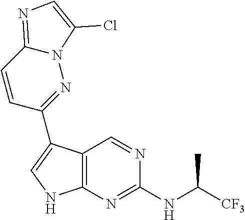 [SMILES: C[C@H](Nc1ncc2c(-c3ccc4ncc(Cl)n4n3)c[nH]c2n1)C(F)(F)F] | 956 |
| 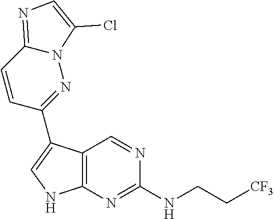 [SMILES: FC(F)(F)CCNc1ncc2c(-c3ccc4ncc(Cl)n4n3)c[nH]c2n1] | 957 |
| 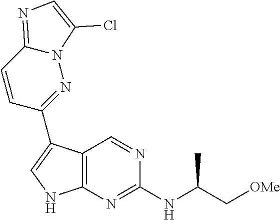 [SMILES: COC[C@H](C)Nc1ncc2c(-c3ccc4ncc(Cl)n4n3)c[nH]c2n1] | 958 |
| 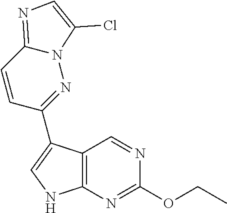 [SMILES: CCOc1ncc2c(-c3ccc4ncc(Cl)n4n3)c[nH]c2n1] | 959 |
| 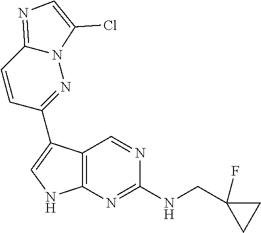 [SMILES: FC1(CNc2ncc3c(-c4ccc5ncc(Cl)n5n4)c[nH]c3n2)CC1] | 960 |
| 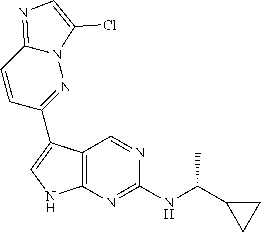 [SMILES: C[C@@H](Nc1ncc2c(-c3ccc4ncc(Cl)n4n3)c[nH]c2n1)C1CC1] | 961 |
| 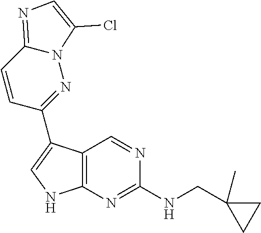 [SMILES: CC1(CNc2ncc3c(-c4ccc5ncc(Cl)n5n4)c[nH]c3n2)CC1] | 962 |
| 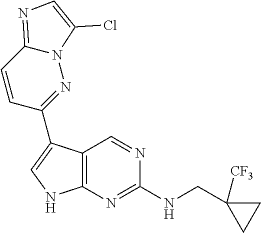 [SMILES: FC(F)(F)C1(CNc2ncc3c(-c4ccc5ncc(Cl)n5n4)c[nH]c3n2)CC1] | 963 |
| 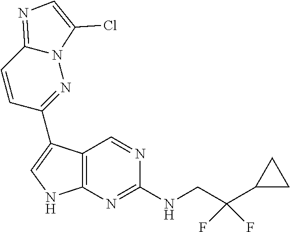 [SMILES: FC(F)(CNc1ncc2c(-c3ccc4ncc(Cl)n4n3)c[nH]c2n1)C1CC1] | 964 |
| 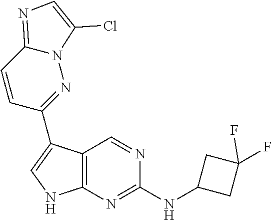 [SMILES: FC1(F)CC(Nc2ncc3c(-c4ccc5ncc(Cl)n5n4)c[nH]c3n2)C1] | 965 |
| 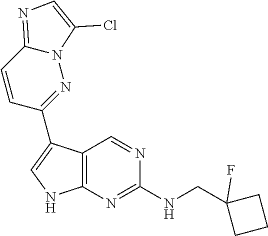 [SMILES: FC1(CNc2ncc3c(-c4ccc5ncc(Cl)n5n4)c[nH]c3n2)CCC1] | 966 |
| 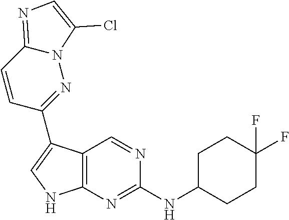 [SMILES: FC1(F)CCC(Nc2ncc3c(-c4ccc5ncc(Cl)n5n4)c[nH]c3n2)CC1] | 967 |
| 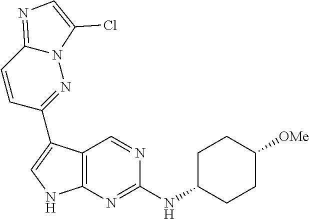 [SMILES: CO[C@H]1CC[C@@H](Nc2ncc3c(-c4ccc5ncc(Cl)n5n4)c[nH]c3n2)CC1] | 968 |
| 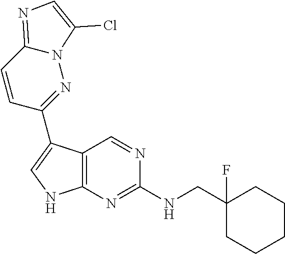 [SMILES: FC1(CNc2ncc3c(-c4ccc5ncc(Cl)n5n4)c[nH]c3n2)CCCCC1] | 969 |
| 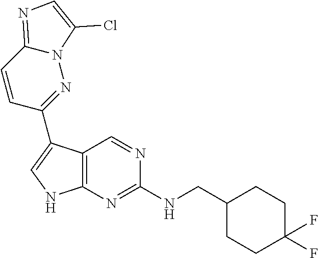 [SMILES: FC1(F)CCC(CNc2ncc3c(-c4ccc5ncc(Cl)n5n4)c[nH]c3n2)CC1] | 970 |
| 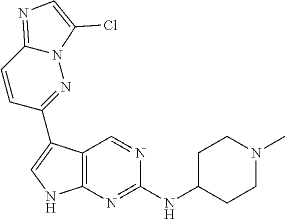 [SMILES: CN1CCC(Nc2ncc3c(-c4ccc5ncc(Cl)n5n4)c[nH]c3n2)CC1] | 971 |
| 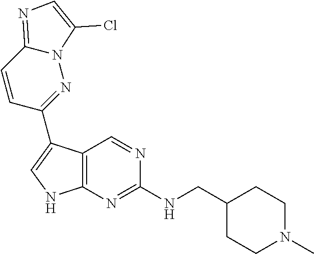 [SMILES: CN1CCC(CNc2ncc3c(-c4ccc5ncc(Cl)n5n4)c[nH]c3n2)CC1] | 972 |
| 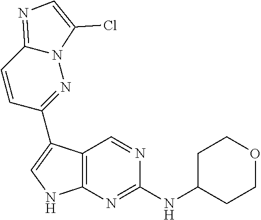 [SMILES: Clc1cnc2ccc(-c3c[nH]c4nc(NC5CCOCC5)ncc34)nn12] | 973 |
| 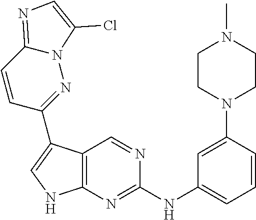 [SMILES: CN1CCN(c2cccc(Nc3ncc4c(-c5ccc6ncc(Cl)n6n5)c[nH]c4n3)c2)CC1] | 974 |
| 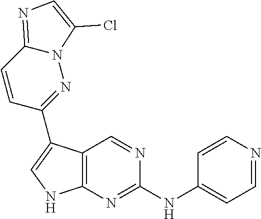 [SMILES: Clc1cnc2ccc(-c3c[nH]c4nc(Nc5ccncc5)ncc34)nn12] | 975 |
| 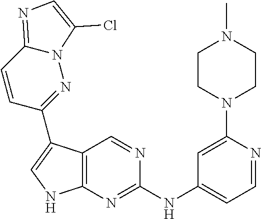 [SMILES: CN1CCN(c2cc(Nc3ncc4c(-c5ccc6ncc(Cl)n6n5)c[nH]c4n3)ccn2)CC1] | 976 |
| 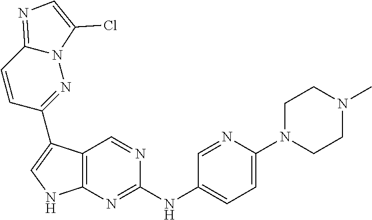 [SMILES: CN1CCN(c2ccc(Nc3ncc4c(-c5ccc6ncc(Cl)n6n5)c[nH]c4n3)cn2)CC1] | 977 |
| 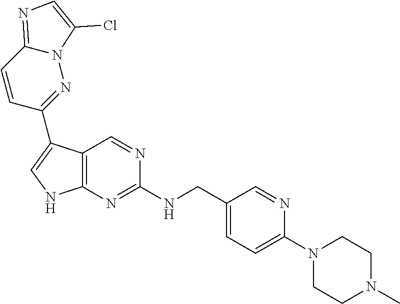 [SMILES: CN1CCN(c2ccc(CNc3ncc4c(-c5ccc6ncc(Cl)n6n5)c[nH]c4n3)cn2)CC1] | 978 |
| 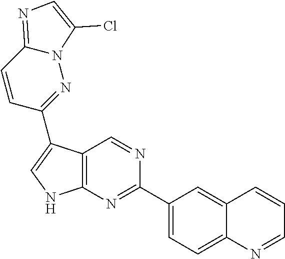 [SMILES: Clc1cnc2ccc(-c3c[nH]c4nc(-c5ccc6ncccc6c5)ncc34)nn12] | 979 |
| 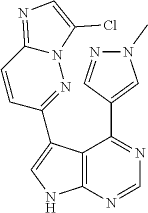 [SMILES: Cn1cc(-c2ncnc3[nH]cc(-c4ccc5ncc(Cl)n5n4)c23)cn1] | 980 |
| 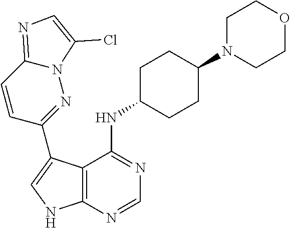 [SMILES: Clc1cnc2ccc(-c3c[nH]c4ncnc(N[C@H]5CC[C@H](N6CCOCC6)CC5)c34)nn12] | 981 |
| 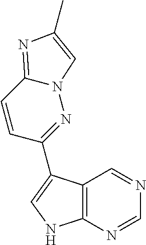 [SMILES: Cc1cn2nc(-c3c[nH]c4ncncc34)ccc2n1] | 982 |
| 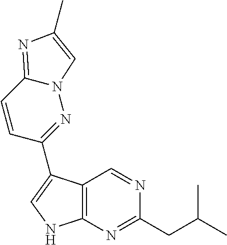 [SMILES: Cc1cn2nc(-c3c[nH]c4nc(CC(C)C)ncc34)ccc2n1] | 983 |
| 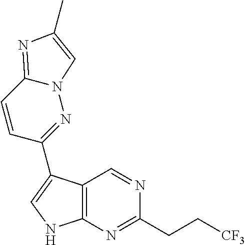 [SMILES: Cc1cn2nc(-c3c[nH]c4nc(CCC(F)(F)F)ncc34)ccc2n1] | 984 |
| 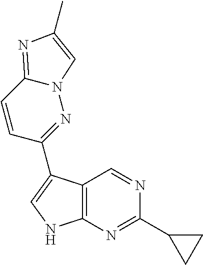 [SMILES: Cc1cn2nc(-c3c[nH]c4nc(C5CC5)ncc34)ccc2n1] | 985 |
| 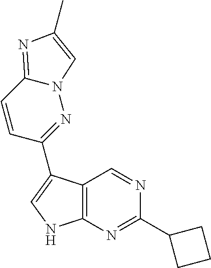 [SMILES: Cc1cn2nc(-c3c[nH]c4nc(C5CCC5)ncc34)ccc2n1] | 986 |
| 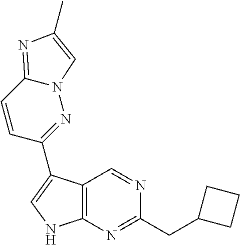 [SMILES: Cc1cn2nc(-c3c[nH]c4nc(CC5CCC5)ncc34)ccc2n1] | 987 |
| 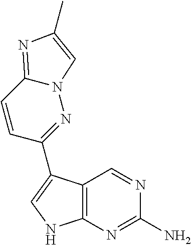 [SMILES: Cc1cn2nc(-c3c[nH]c4nc(N)ncc34)ccc2n1] | 988 |
| 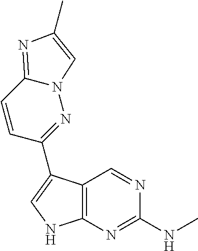 [SMILES: CNc1ncc2c(-c3ccc4nc(C)cn4n3)c[nH]c2n1] | 989 |
| 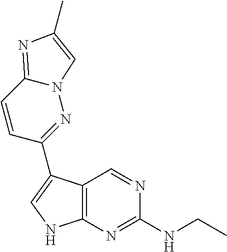 [SMILES: CCNc1ncc2c(-c3ccc4nc(C)cn4n3)c[nH]c2n1] | 990 |
| 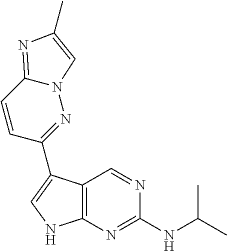 [SMILES: Cc1cn2nc(-c3c[nH]c4nc(NC(C)C)ncc34)ccc2n1] | 991 |
| 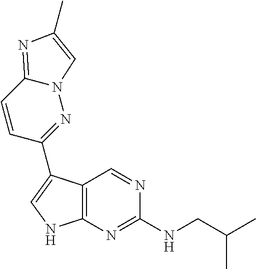 [SMILES: Cc1cn2nc(-c3c[nH]c4nc(NCC(C)C)ncc34)ccc2n1] | 992 |
| 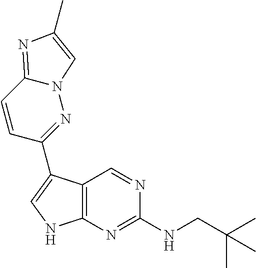 [SMILES: Cc1cn2nc(-c3c[nH]c4nc(NCC(C)(C)C)ncc34)ccc2n1] | 993 |
| 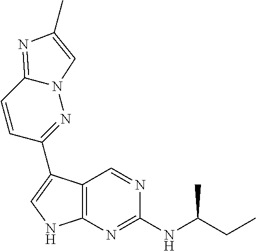 [SMILES: CC[C@H](C)Nc1ncc2c(-c3ccc4nc(C)cn4n3)c[nH]c2n1] | 994 |
| 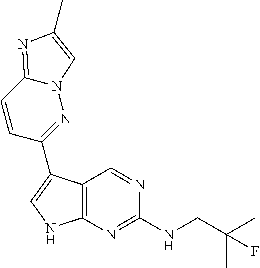 [SMILES: Cc1cn2nc(-c3c[nH]c4nc(NCC(C)(C)F)ncc34)ccc2n1] | 995 |
| 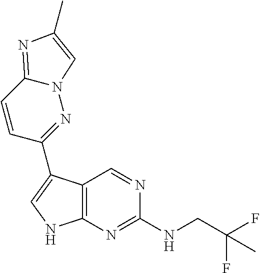 [SMILES: Cc1cn2nc(-c3c[nH]c4nc(NCC(C)(F)F)ncc34)ccc2n1] | 996 |
| 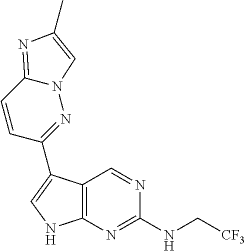 [SMILES: Cc1cn2nc(-c3c[nH]c4nc(NCC(F)(F)F)ncc34)ccc2n1] | 997 |
| 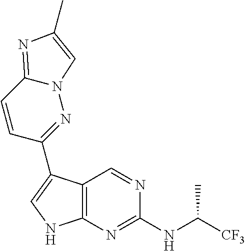 [SMILES: Cc1cn2nc(-c3c[nH]c4nc(N[C@H](C)C(F)(F)F)ncc34)ccc2n1] | 998 |
| 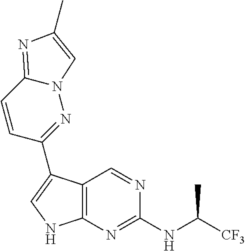 [SMILES: Cc1cn2nc(-c3c[nH]c4nc(N[C@@H](C)C(F)(F)F)ncc34)ccc2n1] | 999 |
| 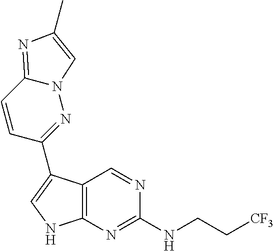 [SMILES: Cc1cn2nc(-c3c[nH]c4nc(NCCC(F)(F)F)ncc34)ccc2n1] | 1000 |
| 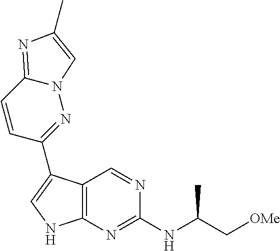 [SMILES: COC[C@H](C)Nc1ncc2c(-c3ccc4nc(C)cn4n3)c[nH]c2n1] | 1001 |
| 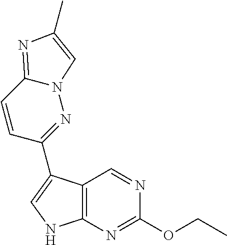 [SMILES: CCOc1ncc2c(-c3ccc4nc(C)cn4n3)c[nH]c2n1] | 1002 |
| 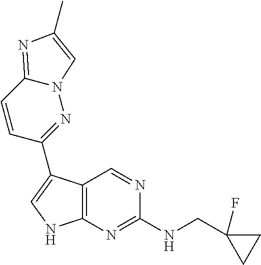 [SMILES: Cc1cn2nc(-c3c[nH]c4nc(NCC5(F)CC5)ncc34)ccc2n1] | 1003 |
| 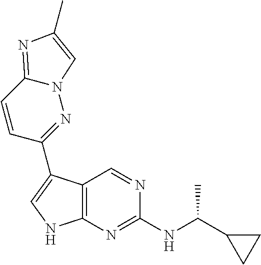 [SMILES: Cc1cn2nc(-c3c[nH]c4nc(N[C@H](C)C5CC5)ncc34)ccc2n1] | 1004 |
| 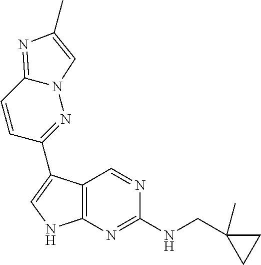 [SMILES: Cc1cn2nc(-c3c[nH]c4nc(NCC5(C)CC5)ncc34)ccc2n1] | 1005 |
| 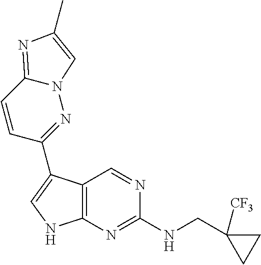 [SMILES: Cc1cn2nc(-c3c[nH]c4nc(NCC5(C(F)(F)F)CC5)ncc34)ccc2n1] | 1006 |
| 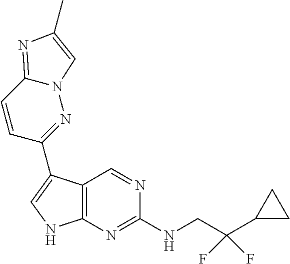 [SMILES: Cc1cn2nc(-c3c[nH]c4nc(NCC(F)(F)C5CC5)ncc34)ccc2n1] | 1007 |
| 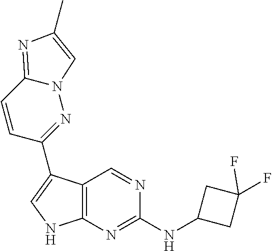 [SMILES: Cc1cn2nc(-c3c[nH]c4nc(NC5CC(F)(F)C5)ncc34)ccc2n1] | 1008 |
| 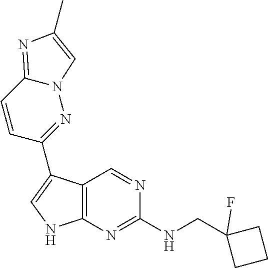 [SMILES: Cc1cn2nc(-c3c[nH]c4nc(NCC5(F)CCC5)ncc34)ccc2n1] | 1009 |
| 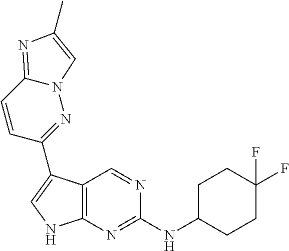 [SMILES: Cc1cn2nc(-c3c[nH]c4nc(NC5CCC(F)(F)CC5)ncc34)ccc2n1] | 1010 |
| 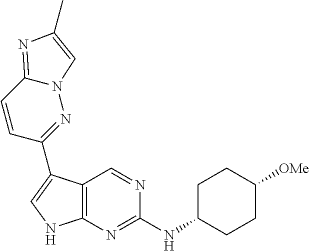 [SMILES: CO[C@H]1CC[C@@H](Nc2ncc3c(-c4ccc5nc(C)cn5n4)c[nH]c3n2)CC1] | 1011 |
| 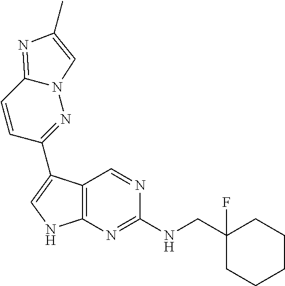 [SMILES: Cc1cn2nc(-c3c[nH]c4nc(NCC5(F)CCCCC5)ncc34)ccc2n1] | 1012 |
| 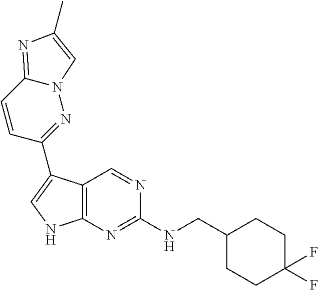 [SMILES: Cc1cn2nc(-c3c[nH]c4nc(NCC5CCC(F)(F)CC5)ncc34)ccc2n1] | 1013 |
| 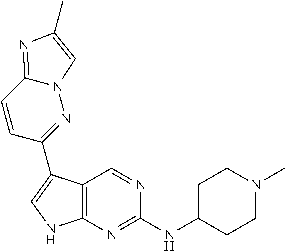 [SMILES: Cc1cn2nc(-c3c[nH]c4nc(NC5CCN(C)CC5)ncc34)ccc2n1] | 1014 |
| 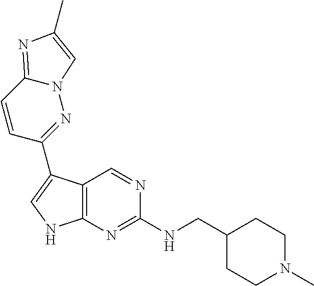 [SMILES: Cc1cn2nc(-c3c[nH]c4nc(NCC5CCN(C)CC5)ncc34)ccc2n1] | 1015 |
| 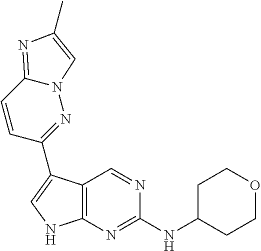 [SMILES: Cc1cn2nc(-c3c[nH]c4nc(NC5CCOCC5)ncc34)ccc2n1] | 1016 |
| 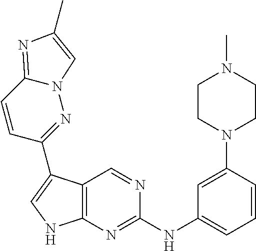 [SMILES: Cc1cn2nc(-c3c[nH]c4nc(Nc5cccc(N6CCN(C)CC6)c5)ncc34)ccc2n1] | 1017 |
| 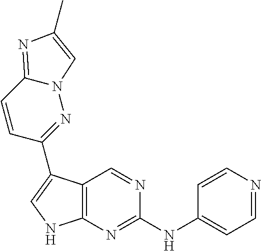 [SMILES: Cc1cn2nc(-c3c[nH]c4nc(Nc5ccncc5)ncc34)ccc2n1] | 1018 |
| 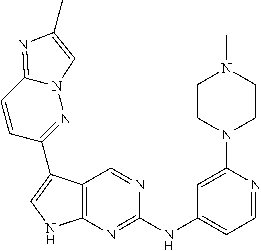 [SMILES: Cc1cn2nc(-c3c[nH]c4nc(Nc5ccnc(N6CCN(C)CC6)c5)ncc34)ccc2n1] | 1019 |
| 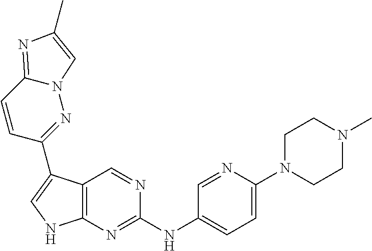 [SMILES: Cc1cn2nc(-c3c[nH]c4nc(Nc5ccc(N6CCN(C)CC6)nc5)ncc34)ccc2n1] | 1020 |
| 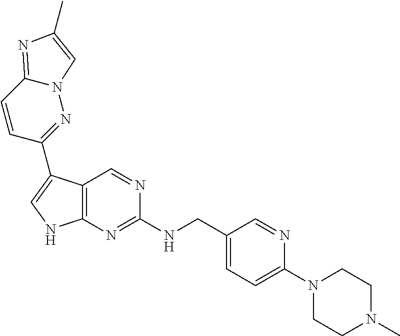 [SMILES: Cc1cn2nc(-c3c[nH]c4nc(NCc5ccc(N6CCN(C)CC6)nc5)ncc34)ccc2n1] | 1021 |
| 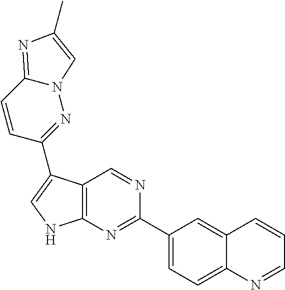 [SMILES: Cc1cn2nc(-c3c[nH]c4nc(-c5ccc6ncccc6c5)ncc34)ccc2n1] | 1022 |
| 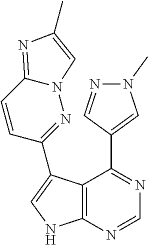 [SMILES: Cc1cn2nc(-c3c[nH]c4ncnc(-c5cnn(C)c5)c34)ccc2n1] | 1023 |
| 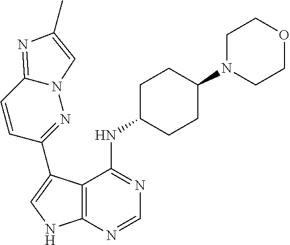 [SMILES: Cc1cn2nc(-c3c[nH]c4ncnc(N[C@H]5CC[C@H](N6CCOCC6)CC5)c34)ccc2n1] | 1024 |
| 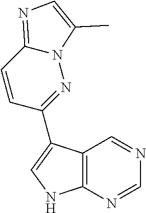 [SMILES: Cc1cnc2ccc(-c3c[nH]c4ncncc34)nn12] | 1025 |
| 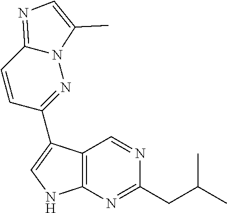 [SMILES: Cc1cnc2ccc(-c3c[nH]c4nc(CC(C)C)ncc34)nn12] | 1026 |
| 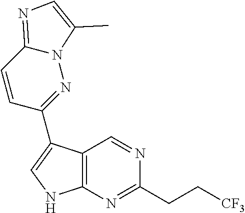 [SMILES: Cc1cnc2ccc(-c3c[nH]c4nc(CCC(F)(F)F)ncc34)nn12] | 1027 |
| 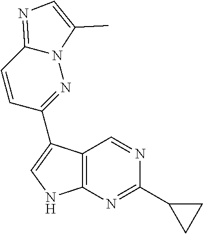 [SMILES: Cc1cnc2ccc(-c3c[nH]c4nc(C5CC5)ncc34)nn12] | 1028 |
| 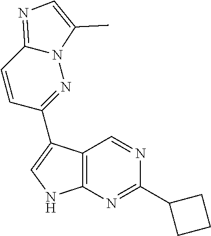 [SMILES: Cc1cnc2ccc(-c3c[nH]c4nc(C5CCC5)ncc34)nn12] | 1029 |
| 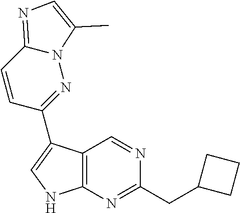 [SMILES: Cc1cnc2ccc(-c3c[nH]c4nc(CC5CCC5)ncc34)nn12] | 1030 |
| 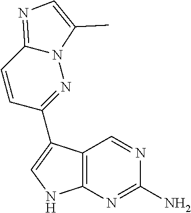 [SMILES: Cc1cnc2ccc(-c3c[nH]c4nc(N)ncc34)nn12] | 1031 |
| 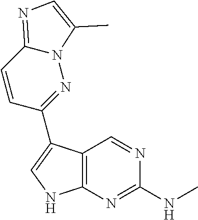 [SMILES: CNc1ncc2c(-c3ccc4ncc(C)n4n3)c[nH]c2n1] | 1032 |
| 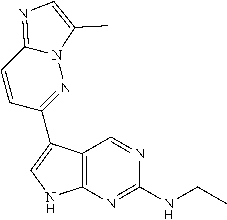 [SMILES: CCNc1ncc2c(-c3ccc4ncc(C)n4n3)c[nH]c2n1] | 1033 |
| 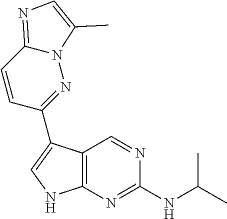 [SMILES: Cc1cnc2ccc(-c3c[nH]c4nc(NC(C)C)ncc34)nn12] | 1034 |
| 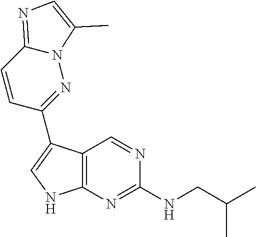 [SMILES: Cc1cnc2ccc(-c3c[nH]c4nc(NCC(C)C)ncc34)nn12] | 1035 |
| 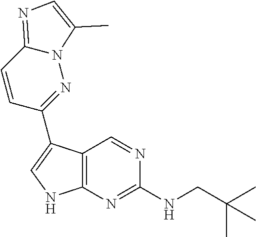 [SMILES: Cc1cnc2ccc(-c3c[nH]c4nc(NCC(C)(C)C)ncc34)nn12] | 1036 |
| 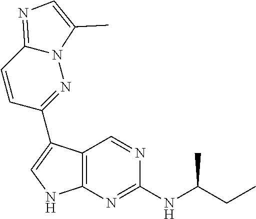 [SMILES: CC[C@H](C)Nc1ncc2c(-c3ccc4ncc(C)n4n3)c[nH]c2n1] | 1037 |
| 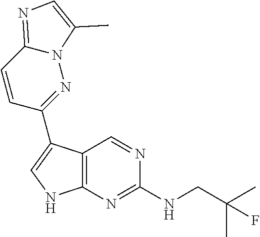 [SMILES: Cc1cnc2ccc(-c3c[nH]c4nc(NCC(C)(C)F)ncc34)nn12] | 1038 |
| 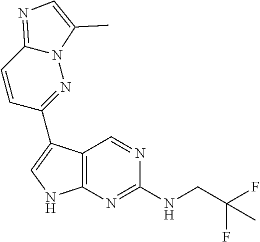 [SMILES: Cc1cnc2ccc(-c3c[nH]c4nc(NCC(C)(F)F)ncc34)nn12] | 1039 |
| 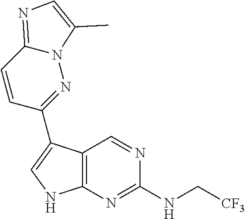 [SMILES: Cc1cnc2ccc(-c3c[nH]c4nc(NCC(F)(F)F)ncc34)nn12] | 1040 |
| 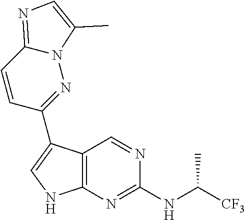 [SMILES: Cc1cnc2ccc(-c3c[nH]c4nc(N[C@H](C)C(F)(F)F)ncc34)nn12] | 1041 |
| 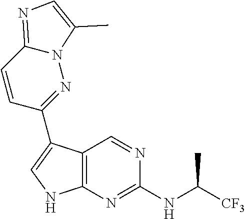 [SMILES: Cc1cnc2ccc(-c3c[nH]c4nc(N[C@@H](C)C(F)(F)F)ncc34)nn12] | 1042 |
| 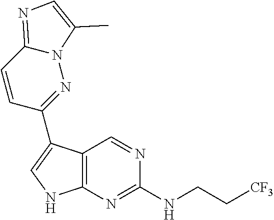 [SMILES: Cc1cnc2ccc(-c3c[nH]c4nc(NCCC(F)(F)F)ncc34)nn12] | 1043 |
| 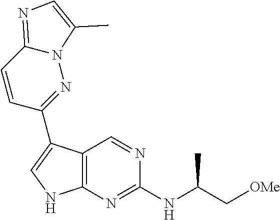 [SMILES: COC[C@H](C)Nc1ncc2c(-c3ccc4ncc(C)n4n3)c[nH]c2n1] | 1044 |
| 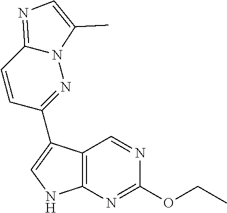 [SMILES: CCOc1ncc2c(-c3ccc4ncc(C)n4n3)c[nH]c2n1] | 1045 |
| 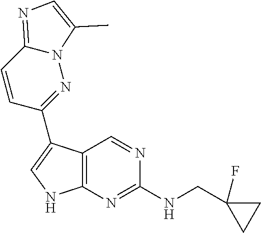 [SMILES: Cc1cnc2ccc(-c3c[nH]c4nc(NCC5(F)CC5)ncc34)nn12] | 1046 |
| 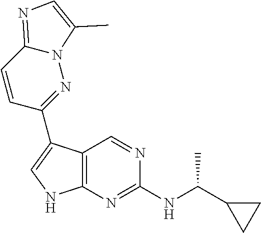 [SMILES: Cc1cnc2ccc(-c3c[nH]c4nc(N[C@H](C)C5CC5)ncc34)nn12] | 1047 |
| 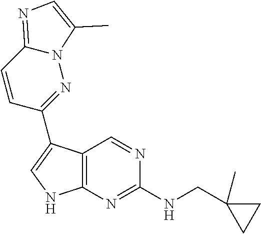 [SMILES: Cc1cnc2ccc(-c3c[nH]c4nc(NCC5(C)CC5)ncc34)nn12] | 1048 |
| 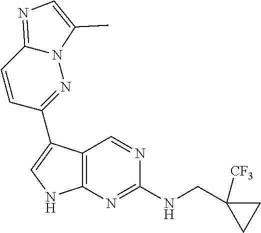 [SMILES: Cc1cnc2ccc(-c3c[nH]c4nc(NCC5(C(F)(F)F)CC5)ncc34)nn12] | 1049 |
| 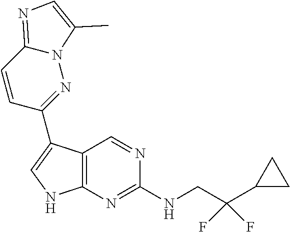 [SMILES: Cc1cnc2ccc(-c3c[nH]c4nc(NCC(F)(F)C5CC5)ncc34)nn12] | 1050 |
| 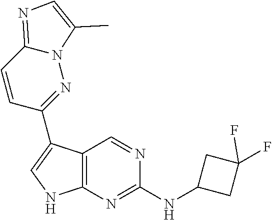 [SMILES: Cc1cnc2ccc(-c3c[nH]c4nc(NC5CC(F)(F)C5)ncc34)nn12] | 1051 |
| 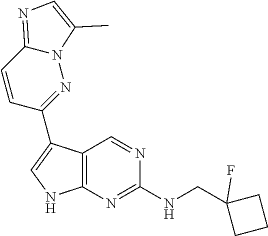 [SMILES: Cc1cnc2ccc(-c3c[nH]c4nc(NCC5(F)CCC5)ncc34)nn12] | 1052 |
| 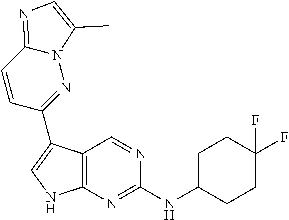 [SMILES: Cc1cnc2ccc(-c3c[nH]c4nc(NC5CCC(F)(F)CC5)ncc34)nn12] | 1053 |
| 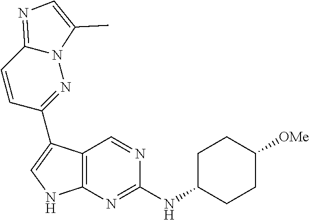 [SMILES: CO[C@H]1CC[C@@H](Nc2ncc3c(-c4ccc5ncc(C)n5n4)c[nH]c3n2)CC1] | 1054 |
| 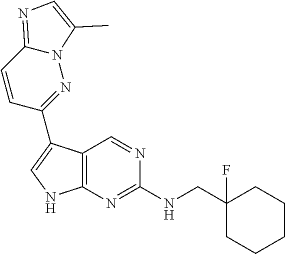 [SMILES: Cc1cnc2ccc(-c3c[nH]c4nc(NCC5(F)CCCCC5)ncc34)nn12] | 1055 |
| 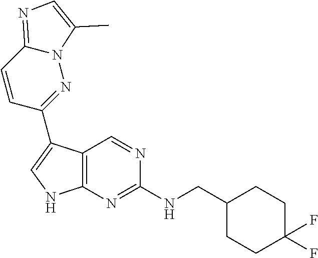 [SMILES: Cc1cnc2ccc(-c3c[nH]c4nc(NCC5CCC(F)(F)CC5)ncc34)nn12] | 1056 |
| 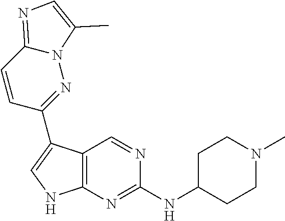 [SMILES: Cc1cnc2ccc(-c3c[nH]c4nc(NC5CCN(C)CC5)ncc34)nn12] | 1057 |
| 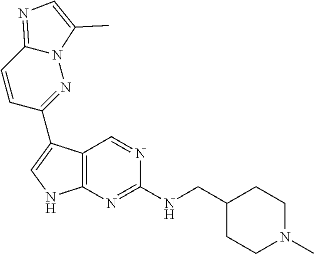 [SMILES: Cc1cnc2ccc(-c3c[nH]c4nc(NCC5CCN(C)CC5)ncc34)nn12] | 1058 |
| 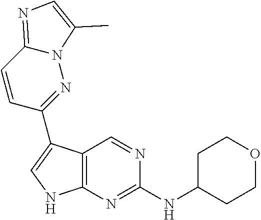 [SMILES: Cc1cnc2ccc(-c3c[nH]c4nc(NC5CCOCC5)ncc34)nn12] | 1059 |
| 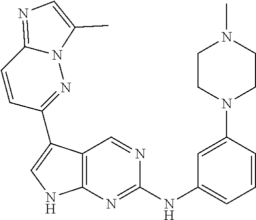 [SMILES: Cc1cnc2ccc(-c3c[nH]c4nc(Nc5cccc(N6CCN(C)CC6)c5)ncc34)nn12] | 1060 |
| 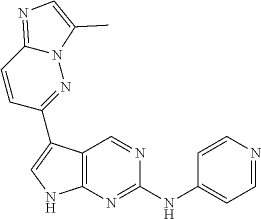 [SMILES: Cc1cnc2ccc(-c3c[nH]c4nc(Nc5ccncc5)ncc34)nn12] | 1061 |
| 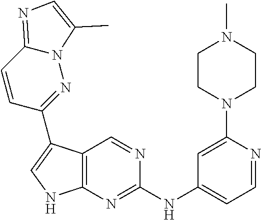 [SMILES: Cc1cnc2ccc(-c3c[nH]c4nc(Nc5ccnc(N6CCN(C)CC6)c5)ncc34)nn12] | 1062 |
| 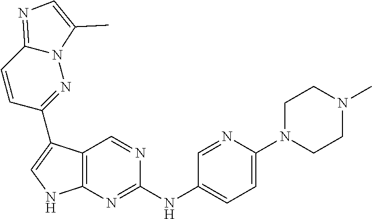 [SMILES: Cc1cnc2ccc(-c3c[nH]c4nc(Nc5ccc(N6CCN(C)CC6)nc5)ncc34)nn12] | 1063 |
| 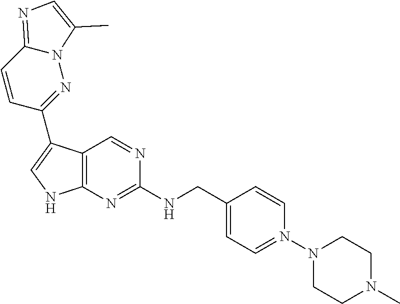 | 1064 |
| 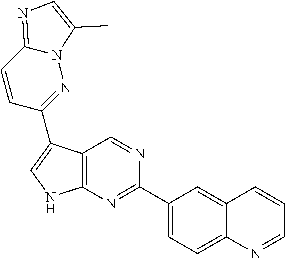 [SMILES: Cc1cnc2ccc(-c3c[nH]c4nc(-c5ccc6ncccc6c5)ncc34)nn12] | 1065 |
| 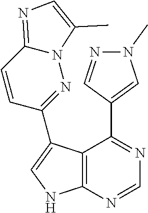 [SMILES: Cc1cnc2ccc(-c3c[nH]c4ncnc(-c5cnn(C)c5)c34)nn12] | 1066 |
| 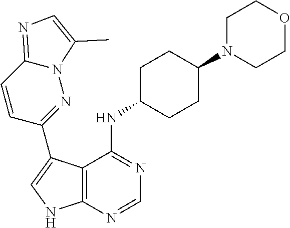 [SMILES: Cc1cnc2ccc(-c3c[nH]c4ncnc(N[C@H]5CC[C@H](N6CCOCC6)CC5)c34)nn12] | 1067 |
| 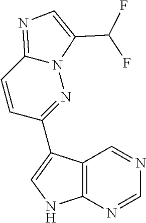 [SMILES: FC(F)c1cnc2ccc(-c3c[nH]c4ncncc34)nn12] | 1068 |
| 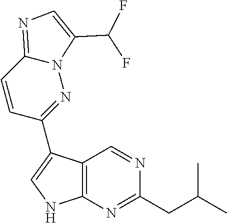 [SMILES: CC(C)Cc1ncc2c(-c3ccc4ncc(C(F)F)n4n3)c[nH]c2n1] | 1069 |
| 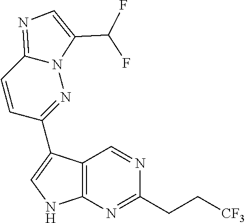 [SMILES: FC(F)c1cnc2ccc(-c3c[nH]c4nc(CCC(F)(F)F)ncc34)nn12] | 1070 |
| 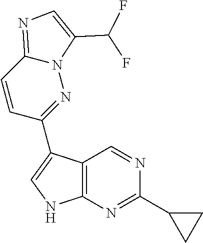 [SMILES: FC(F)c1cnc2ccc(-c3c[nH]c4nc(C5CC5)ncc34)nn12] | 1071 |
| 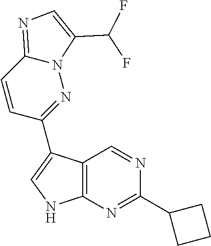 [SMILES: FC(F)c1cnc2ccc(-c3c[nH]c4nc(C5CCC5)ncc34)nn12] | 1072 |
| 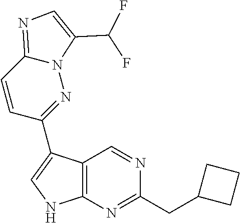 [SMILES: FC(F)c1cnc2ccc(-c3c[nH]c4nc(CC5CCC5)ncc34)nn12] | 1073 |
| 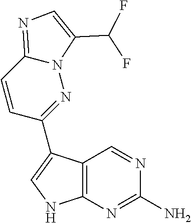 [SMILES: Nc1ncc2c(-c3ccc4ncc(C(F)F)n4n3)c[nH]c2n1] | 1074 |
| 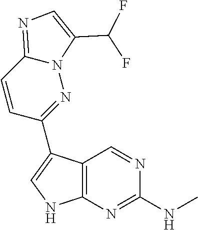 [SMILES: CNc1ncc2c(-c3ccc4ncc(C(F)F)n4n3)c[nH]c2n1] | 1075 |
| 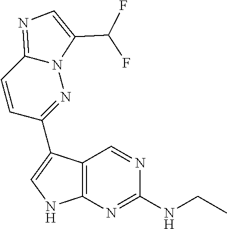 [SMILES: CCNc1ncc2c(-c3ccc4ncc(C(F)F)n4n3)c[nH]c2n1] | 1076 |
| 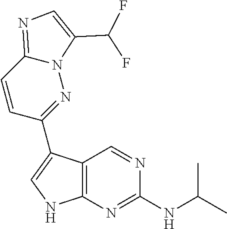 [SMILES: CC(C)Nc1ncc2c(-c3ccc4ncc(C(F)F)n4n3)c[nH]c2n1] | 1077 |
| 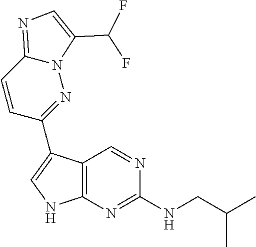 [SMILES: CC(C)CNc1ncc2c(-c3ccc4ncc(C(F)F)n4n3)c[nH]c2n1] | 1078 |
| 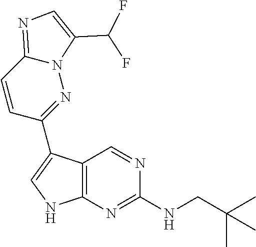 [SMILES: CC(C)(C)CNc1ncc2c(-c3ccc4ncc(C(F)F)n4n3)c[nH]c2n1] | 1079 |
| 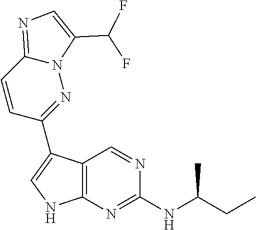 [SMILES: CC[C@H](C)Nc1ncc2c(-c3ccc4ncc(C(F)F)n4n3)c[nH]c2n1] | 1080 |
| 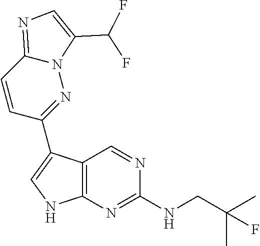 [SMILES: CC(C)(F)CNc1ncc2c(-c3ccc4ncc(C(F)F)n4n3)c[nH]c2n1] | 1081 |
| 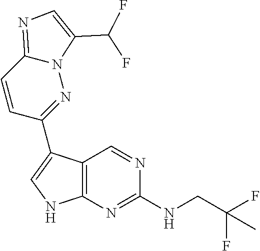 [SMILES: CC(F)(F)CNc1ncc2c(-c3ccc4ncc(C(F)F)n4n3)c[nH]c2n1] | 1082 |
| 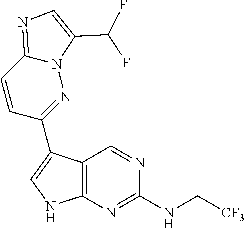 [SMILES: FC(F)c1cnc2ccc(-c3c[nH]c4nc(NCC(F)(F)F)ncc34)nn12] | 1083 |
| 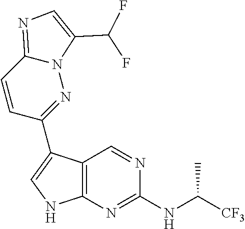 [SMILES: C[C@@H](Nc1ncc2c(-c3ccc4ncc(C(F)F)n4n3)c[nH]c2n1)C(F)(F)F] | 1084 |
| 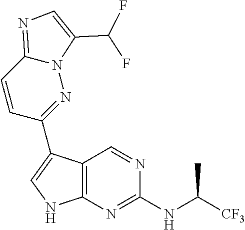 [SMILES: C[C@H](Nc1ncc2c(-c3ccc4ncc(C(F)F)n4n3)c[nH]c2n1)C(F)(F)F] | 1085 |
| 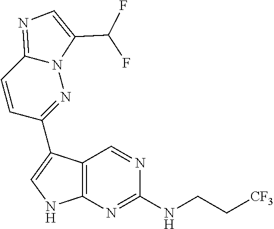 [SMILES: FC(F)c1cnc2ccc(-c3c[nH]c4nc(NCCC(F)(F)F)ncc34)nn12] | 1086 |
| 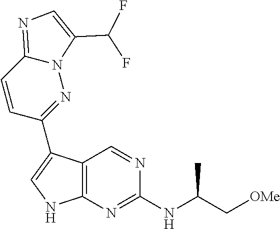 [SMILES: COC[C@H](C)Nc1ncc2c(-c3ccc4ncc(C(F)F)n4n3)c[nH]c2n1] | 1087 |
| 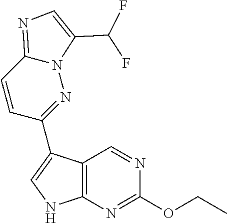 [SMILES: CCOc1ncc2c(-c3ccc4ncc(C(F)F)n4n3)c[nH]c2n1] | 1088 |
| 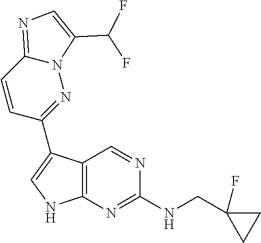 [SMILES: FC(F)c1cnc2ccc(-c3c[nH]c4nc(NCC5(F)CC5)ncc34)nn12] | 1089 |
| 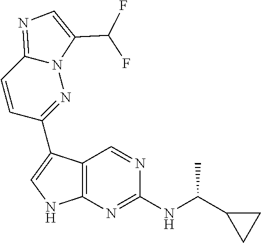 [SMILES: C[C@@H](Nc1ncc2c(-c3ccc4ncc(C(F)F)n4n3)c[nH]c2n1)C1CC1] | 1090 |
| 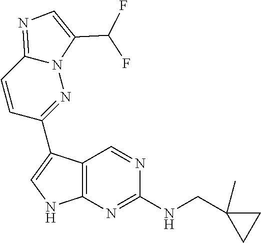 [SMILES: CC1(CNc2ncc3c(-c4ccc5ncc(C(F)F)n5n4)c[nH]c3n2)CC1] | 1091 |
| 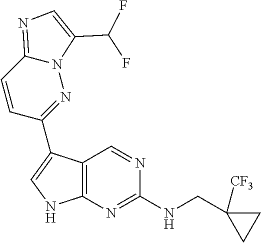 [SMILES: FC(F)c1cnc2ccc(-c3c[nH]c4nc(NCC5(C(F)(F)F)CC5)ncc34)nn12] | 1092 |
| 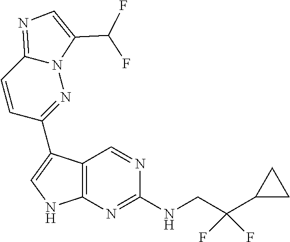 [SMILES: FC(F)c1cnc2ccc(-c3c[nH]c4nc(NCC(F)(F)C5CC5)ncc34)nn12] | 1093 |
| 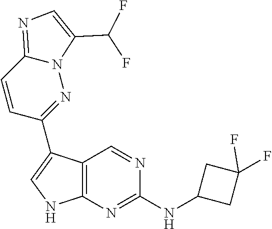 [SMILES: FC(F)c1cnc2ccc(-c3c[nH]c4nc(NC5CC(F)(F)C5)ncc34)nn12] | 1094 |
| 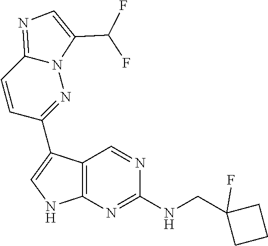 [SMILES: FC(F)c1cnc2ccc(-c3c[nH]c4nc(NCC5(F)CCC5)ncc34)nn12] | 1095 |
| 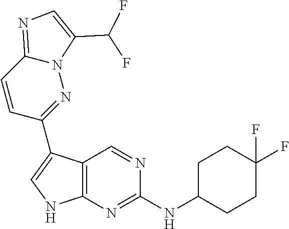 [SMILES: FC(F)c1cnc2ccc(-c3c[nH]c4nc(NC5CCC(F)(F)CC5)ncc34)nn12] | 1096 |
| 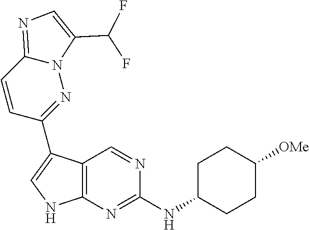 [SMILES: CO[C@H]1CC[C@@H](Nc2ncc3c(-c4ccc5ncc(C(F)F)n5n4)c[nH]c3n2)CC1] | 1097 |
| 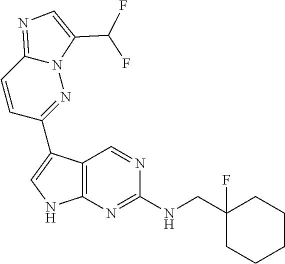 [SMILES: FC(F)c1cnc2ccc(-c3c[nH]c4nc(NCC5(F)CCCCC5)ncc34)nn12] | 1098 |
| 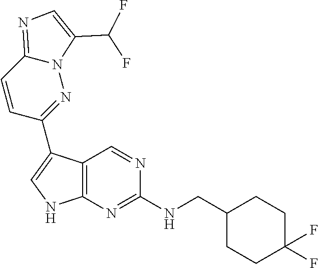 [SMILES: FC(F)c1cnc2ccc(-c3c[nH]c4nc(NCC5CCC(F)(F)CC5)ncc34)nn12] | 1099 |
| 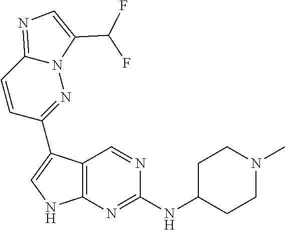 [SMILES: CN1CCC(Nc2ncc3c(-c4ccc5ncc(C(F)F)n5n4)c[nH]c3n2)CC1] | 1100 |
| 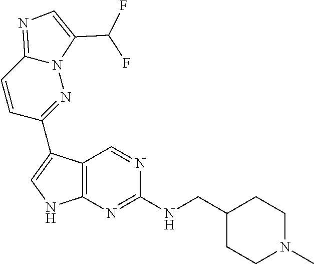 [SMILES: CN1CCC(CNc2ncc3c(-c4ccc5ncc(C(F)F)n5n4)c[nH]c3n2)CC1] | 1101 |
| 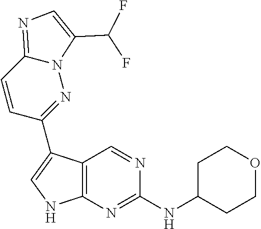 [SMILES: FC(F)c1cnc2ccc(-c3c[nH]c4nc(NC5CCOCC5)ncc34)nn12] | 1102 |
| 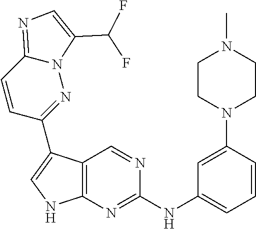 [SMILES: CN1CCN(c2cccc(Nc3ncc4c(-c5ccc6ncc(C(F)F)n6n5)c[nH]c4n3)c2)CC1] | 1103 |
| 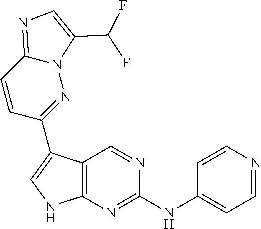 [SMILES: FC(F)c1cnc2ccc(-c3c[nH]c4nc(Nc5ccncc5)ncc34)nn12] | 1104 |
| 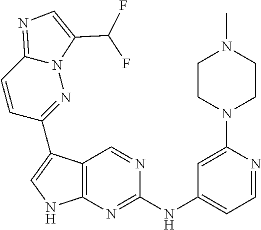 [SMILES: CN1CCN(c2cc(Nc3ncc4c(-c5ccc6ncc(C(F)F)n6n5)c[nH]c4n3)ccn2)CC1] | 1105 |
| 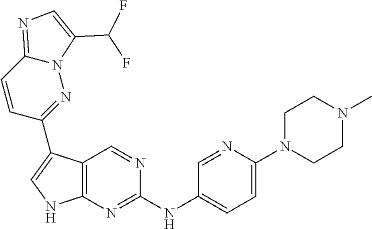 [SMILES: CN1CCN(c2ccc(Nc3ncc4c(-c5ccc6ncc(C(F)F)n6n5)c[nH]c4n3)cn2)CC1] | 1106 |
| 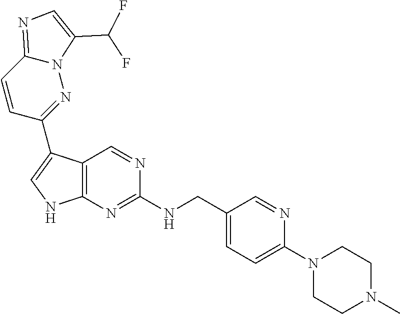 [SMILES: CN1CCN(c2ccc(CNc3ncc4c(-c5ccc6ncc(C(F)F)n6n5)c[nH]c4n3)cn2)CC1] | 1107 |
| 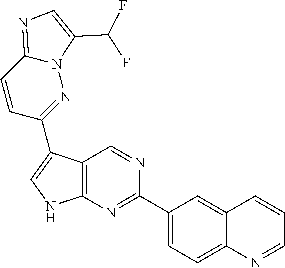 [SMILES: FC(F)c1cnc2ccc(-c3c[nH]c4nc(-c5ccc6ncccc6c5)ncc34)nn12] | 1108 |
| 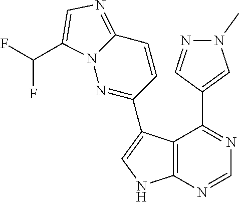 [SMILES: Cn1cc(-c2ncnc3[nH]cc(-c4ccc5ncc(C(F)F)n5n4)c23)cn1] | 1109 |
| 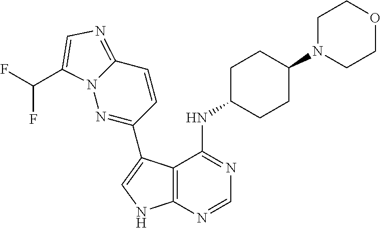 [SMILES: FC(F)c1cnc2ccc(-c3c[nH]c4ncnc(N[C@H]5CC[C@H](N6CCOCC6)CC5)c34)nn12] | 1110 |
| 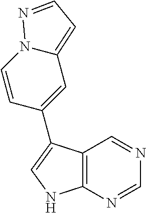 [SMILES: c1ncc2c(-c3ccn4nccc4c3)c[nH]c2n1] | 1111 |
| 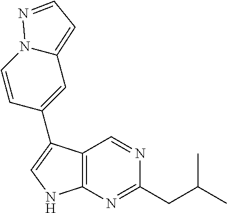 [SMILES: CC(C)Cc1ncc2c(-c3ccn4nccc4c3)c[nH]c2n1] | 1112 |
| 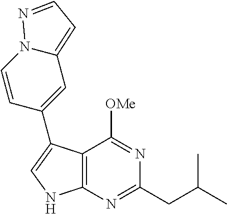 [SMILES: COc1nc(CC(C)C)nc2[nH]cc(-c3ccn4nccc4c3)c12] | 1113 |
| 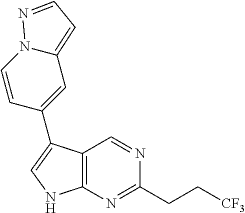 [SMILES: FC(F)(F)CCc1ncc2c(-c3ccn4nccc4c3)c[nH]c2n1] | 1114 |
| 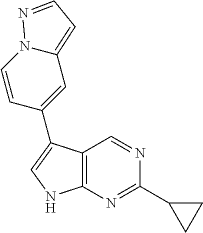 [SMILES: c1cc2cc(-c3c[nH]c4nc(C5CC5)ncc34)ccn2n1] | 1115 |
| 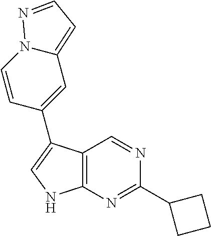 [SMILES: c1cc2cc(-c3c[nH]c4nc(C5CCC5)ncc34)ccn2n1] | 1116 |
| 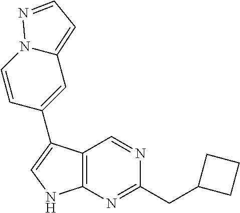 [SMILES: c1cc2cc(-c3c[nH]c4nc(CC5CCC5)ncc34)ccn2n1] | 1117 |
| 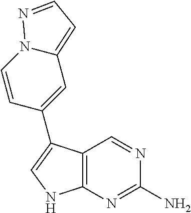 [SMILES: Nc1ncc2c(-c3ccn4nccc4c3)c[nH]c2n1] | 1118 |
| 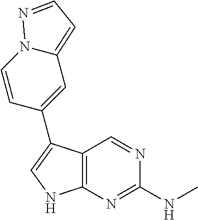 [SMILES: CNc1ncc2c(-c3ccn4nccc4c3)c[nH]c2n1] | 1119 |
| 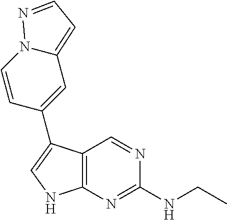 [SMILES: CCNc1ncc2c(-c3ccn4nccc4c3)c[nH]c2n1] | 1120 |
| 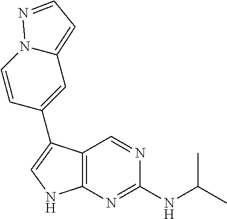 [SMILES: CC(C)Nc1ncc2c(-c3ccn4nccc4c3)c[nH]c2n1] | 1121 |
| 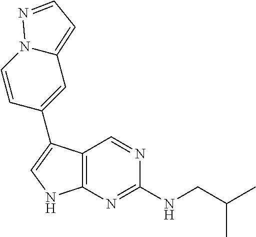 [SMILES: CC(C)CNc1ncc2c(-c3ccn4nccc4c3)c[nH]c2n1] | 1122 |
| 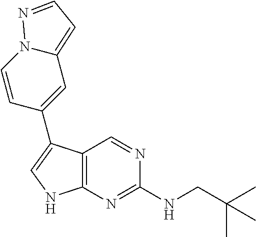 [SMILES: CC(C)(C)CNc1ncc2c(-c3ccn4nccc4c3)c[nH]c2n1] | 1123 |
| 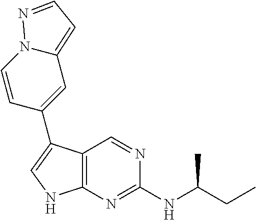 [SMILES: CC[C@H](C)Nc1ncc2c(-c3ccn4nccc4c3)c[nH]c2n1] | 1124 |
| 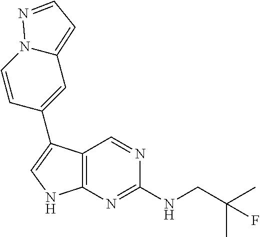 [SMILES: CC(C)(F)CNc1ncc2c(-c3ccn4nccc4c3)c[nH]c2n1] | 1125 |
| 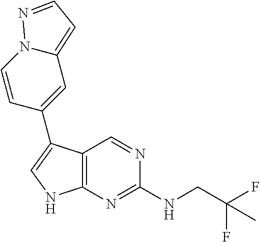 [SMILES: CC(F)(F)CNc1ncc2c(-c3ccn4nccc4c3)c[nH]c2n1] | 1126 |
| 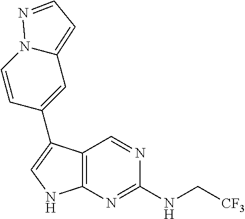 [SMILES: FC(F)(F)CNc1ncc2c(-c3ccn4nccc4c3)c[nH]c2n1] | 1127 |
| 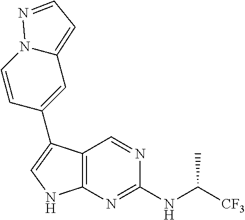 [SMILES: C[C@@H](Nc1ncc2c(-c3ccn4nccc4c3)c[nH]c2n1)C(F)(F)F] | 1128 |
| 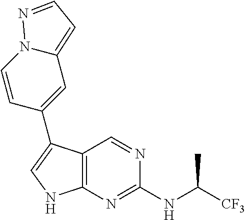 [SMILES: C[C@H](Nc1ncc2c(-c3ccn4nccc4c3)c[nH]c2n1)C(F)(F)F] | 1129 |
| 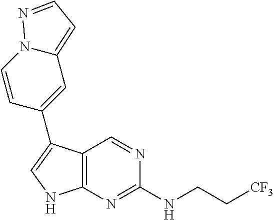 [SMILES: FC(F)(F)CCNc1ncc2c(-c3ccn4nccc4c3)c[nH]c2n1] | 1130 |
| 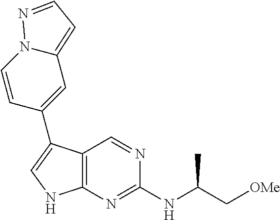 [SMILES: COC[C@H](C)Nc1ncc2c(-c3ccn4nccc4c3)c[nH]c2n1] | 1131 |
| 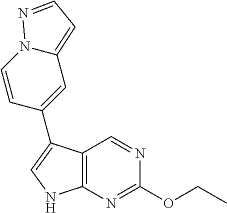 [SMILES: CCOc1ncc2c(-c3ccn4nccc4c3)c[nH]c2n1] | 1132 |
| 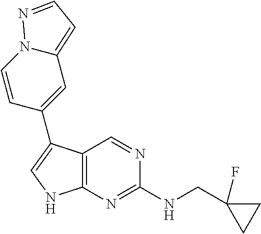 [SMILES: FC1(CNc2ncc3c(-c4ccn5nccc5c4)c[nH]c3n2)CC1] | 1133 |
| 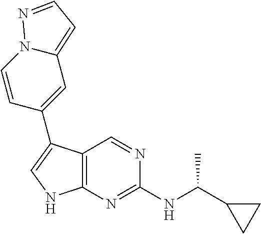 [SMILES: C[C@@H](Nc1ncc2c(-c3ccn4nccc4c3)c[nH]c2n1)C1CC1] | 1134 |
| 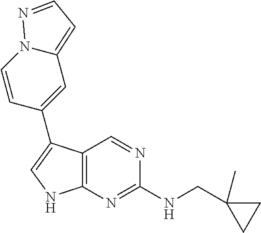 [SMILES: CC1(CNc2ncc3c(-c4ccn5nccc5c4)c[nH]c3n2)CC1] | 1135 |
| 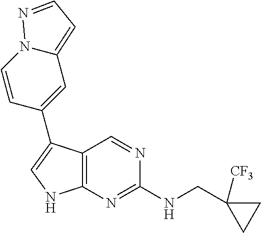 [SMILES: FC(F)(F)C1(CNc2ncc3c(-c4ccn5nccc5c4)c[nH]c3n2)CC1] | 1136 |
| 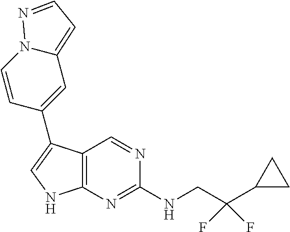 [SMILES: FC(F)(CNc1ncc2c(-c3ccn4nccc4c3)c[nH]c2n1)C1CC1] | 1137 |
| 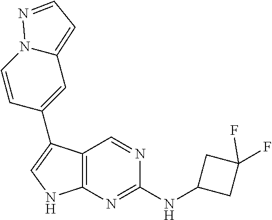 [SMILES: FC1(F)CC(Nc2ncc3c(-c4ccn5nccc5c4)c[nH]c3n2)C1] | 1138 |
| 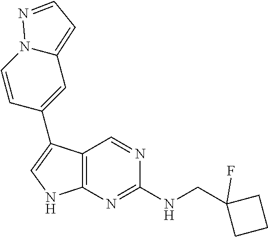 [SMILES: FC1(CNc2ncc3c(-c4ccn5nccc5c4)c[nH]c3n2)CCC1] | 1139 |
| 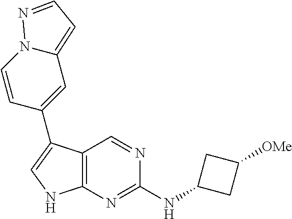 [SMILES: CO[C@H]1C[C@@H](Nc2ncc3c(-c4ccn5nccc5c4)c[nH]c3n2)C1] | 1140 |
| 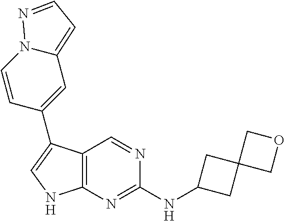 [SMILES: c1cc2cc(-c3c[nH]c4nc(NC5CC6(COC6)C5)ncc34)ccn2n1] | 1141 |
| 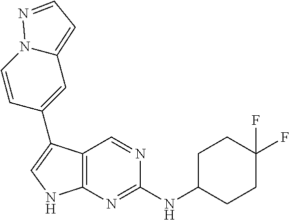 [SMILES: FC1(F)CCC(Nc2ncc3c(-c4ccn5nccc5c4)c[nH]c3n2)CC1] | 1142 |
| 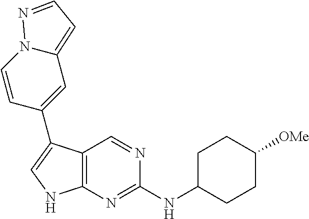 | 1143 |
| 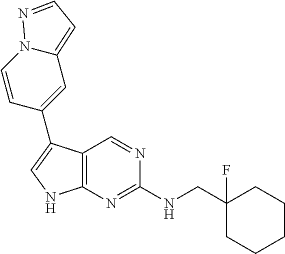 [SMILES: FC1(CNc2ncc3c(-c4ccn5nccc5c4)c[nH]c3n2)CCCCC1] | 1144 |
| 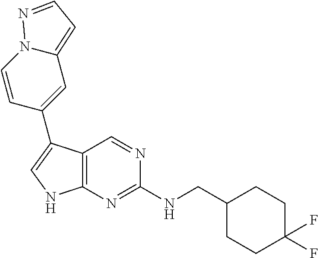 [SMILES: FC1(F)CCC(CNc2ncc3c(-c4ccn5nccc5c4)c[nH]c3n2)CC1] | 1145 |
| 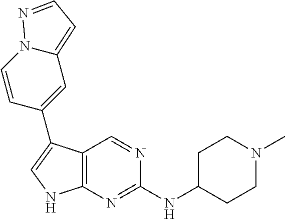 [SMILES: CN1CCC(Nc2ncc3c(-c4ccn5nccc5c4)c[nH]c3n2)CC1] | 1146 |
| 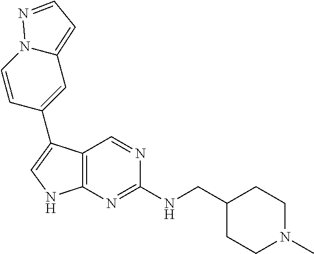 [SMILES: CN1CCC(CNc2ncc3c(-c4ccn5nccc5c4)c[nH]c3n2)CC1] | 1147 |
| 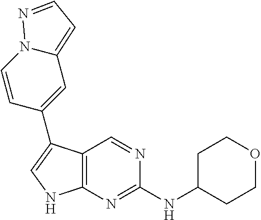 [SMILES: c1cc2cc(-c3c[nH]c4nc(NC5CCOCC5)ncc34)ccn2n1] | 1148 |
| 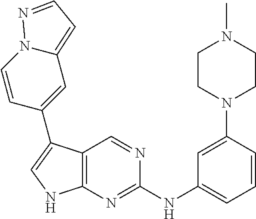 [SMILES: CN1CCN(c2cccc(Nc3ncc4c(-c5ccn6nccc6c5)c[nH]c4n3)c2)CC1] | 1149 |
| 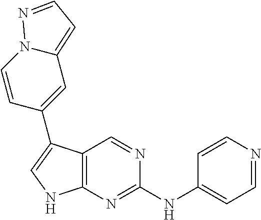 [SMILES: c1cc(Nc2ncc3c(-c4ccn5nccc5c4)c[nH]c3n2)ccn1] | 1150 |
| 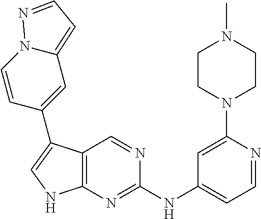 [SMILES: CN1CCN(c2cc(Nc3ncc4c(-c5ccn6nccc6c5)c[nH]c4n3)ccn2)CC1] | 1151 |
| 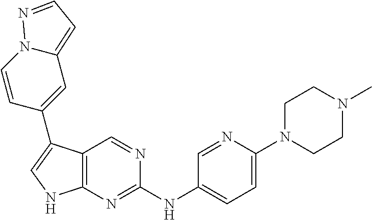 [SMILES: CN1CCN(c2ccc(Nc3ncc4c(-c5ccn6nccc6c5)c[nH]c4n3)cn2)CC1] | 1152 |
| 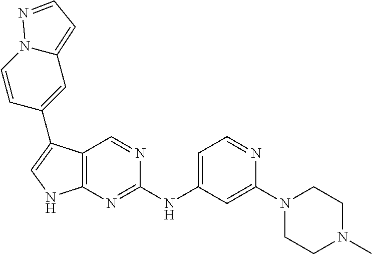 [SMILES: CN1CCN(c2cc(Nc3ncc4c(-c5ccn6nccc6c5)c[nH]c4n3)ccn2)CC1] | 1153 |
| 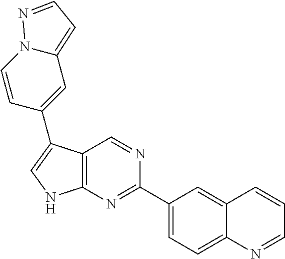 [SMILES: c1cnc2ccc(-c3ncc4c(-c5ccn6nccc6c5)c[nH]c4n3)cc2c1] | 1154 |
| 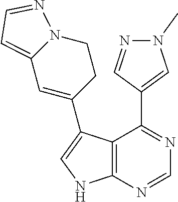 [SMILES: Cn1cc(-c2ncnc3[nH]cc(C4=Cc5ccnn5CC4)c23)cn1] | 1155 |
| 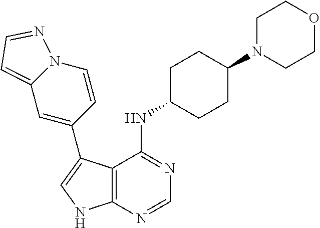 [SMILES: c1nc(N[C@H]2CC[C@H](N3CCOCC3)CC2)c2c(-c3ccn4nccc4c3)c[nH]c2n1] | 1156 |
| 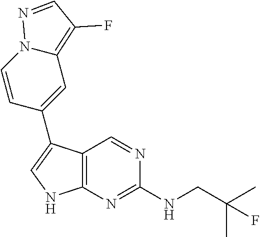 [SMILES: CC(C)(F)CNc1ncc2c(-c3ccn4ncc(F)c4c3)c[nH]c2n1] | 1157 |
| 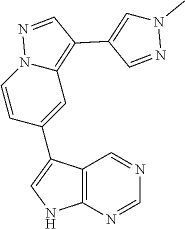 [SMILES: Cn1cc(-c2cnn3ccc(-c4c[nH]c5ncncc45)cc23)cn1] | 1158 |
| 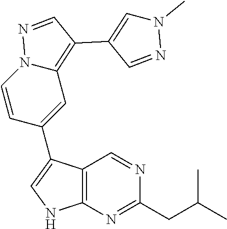 [SMILES: CC(C)Cc1ncc2c(-c3ccn4ncc(-c5cnn(C)c5)c4c3)c[nH]c2n1] | 1159 |
| 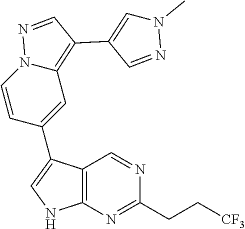 [SMILES: Cn1cc(-c2cnn3ccc(-c4c[nH]c5nc(CCC(F)(F)F)ncc45)cc23)cn1] | 1160 |
| 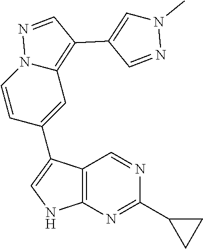 [SMILES: Cn1cc(-c2cnn3ccc(-c4c[nH]c5nc(C6CC6)ncc45)cc23)cn1] | 1161 |
| 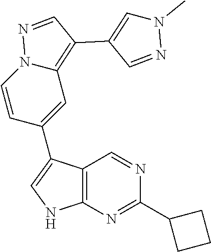 [SMILES: Cn1cc(-c2cnn3ccc(-c4c[nH]c5nc(C6CCC6)ncc45)cc23)cn1] | 1162 |
| 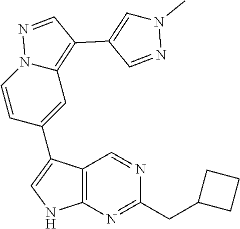 [SMILES: Cn1cc(-c2cnn3ccc(-c4c[nH]c5nc(CC6CCC6)ncc45)cc23)cn1] | 1163 |
| 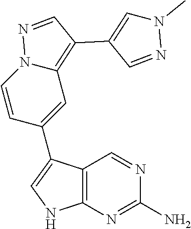 [SMILES: Cn1cc(-c2cnn3ccc(-c4c[nH]c5nc(N)ncc45)cc23)cn1] | 1164 |
| 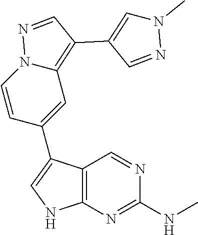 [SMILES: CNc1ncc2c(-c3ccn4ncc(-c5cnn(C)c5)c4c3)c[nH]c2n1] | 1165 |
| 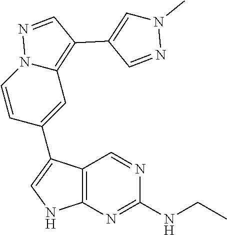 [SMILES: CCNc1ncc2c(-c3ccn4ncc(-c5cnn(C)c5)c4c3)c[nH]c2n1] | 1166 |
| 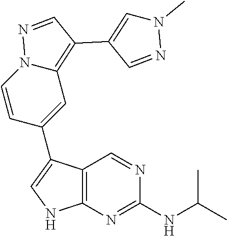 [SMILES: CC(C)Nc1ncc2c(-c3ccn4ncc(-c5cnn(C)c5)c4c3)c[nH]c2n1] | 1167 |
| 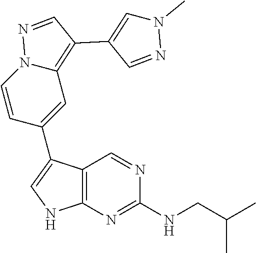 [SMILES: CC(C)CNc1ncc2c(-c3ccn4ncc(-c5cnn(C)c5)c4c3)c[nH]c2n1] | 1168 |
| 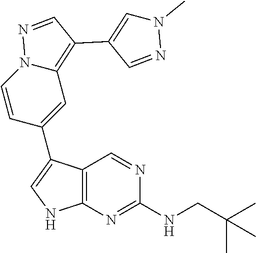 [SMILES: Cn1cc(-c2cnn3ccc(-c4c[nH]c5nc(NCC(C)(C)C)ncc45)cc23)cn1] | 1169 |
| 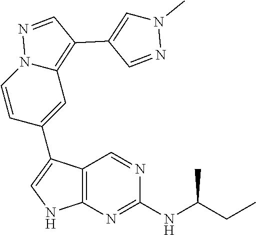 [SMILES: CC[C@H](C)Nc1ncc2c(-c3ccn4ncc(-c5cnn(C)c5)c4c3)c[nH]c2n1] | 1170 |
| 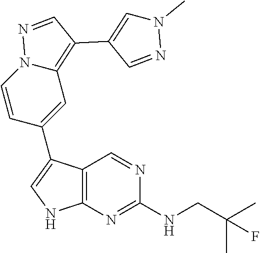 [SMILES: Cn1cc(-c2cnn3ccc(-c4c[nH]c5nc(NCC(C)(C)F)ncc45)cc23)cn1] | 1171 |
| 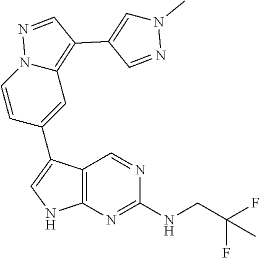 [SMILES: Cn1cc(-c2cnn3ccc(-c4c[nH]c5nc(NCC(C)(F)F)ncc45)cc23)cn1] | 1172 |
| 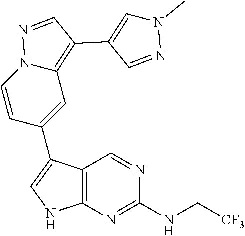 [SMILES: Cn1cc(-c2cnn3ccc(-c4c[nH]c5nc(NCC(F)(F)F)ncc45)cc23)cn1] | 1173 |
| 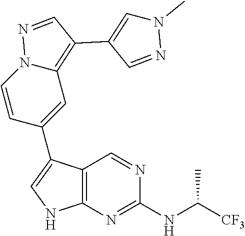 [SMILES: C[C@@H](Nc1ncc2c(-c3ccn4ncc(-c5cnn(C)c5)c4c3)c[nH]c2n1)C(F)(F)F] | 1174 |
| 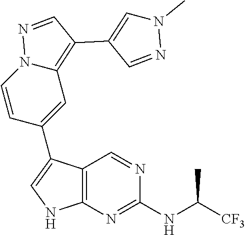 [SMILES: C[C@H](Nc1ncc2c(-c3ccn4ncc(-c5cnn(C)c5)c4c3)c[nH]c2n1)C(F)(F)F] | 1175 |
| 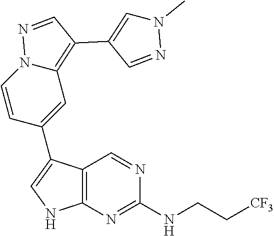 [SMILES: Cn1cc(-c2cnn3ccc(-c4c[nH]c5nc(NCCC(F)(F)F)ncc45)cc23)cn1] | 1176 |
| 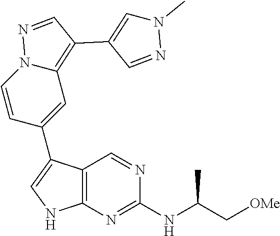 [SMILES: COC[C@H](C)Nc1ncc2c(-c3ccn4ncc(-c5cnn(C)c5)c4c3)c[nH]c2n1] | 1177 |
| 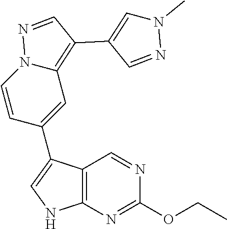 [SMILES: CCOc1ncc2c(-c3ccn4ncc(-c5cnn(C)c5)c4c3)c[nH]c2n1] | 1178 |
| 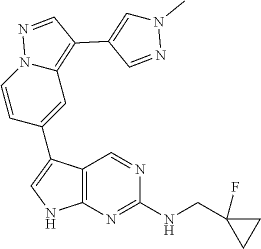 [SMILES: Cn1cc(-c2cnn3ccc(-c4c[nH]c5nc(NCC6(F)CC6)ncc45)cc23)cn1] | 1179 |
| 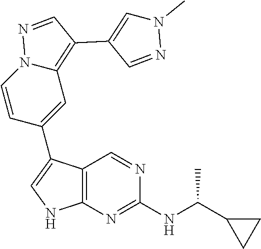 [SMILES: C[C@@H](Nc1ncc2c(-c3ccn4ncc(-c5cnn(C)c5)c4c3)c[nH]c2n1)C1CC1] | 1180 |
| 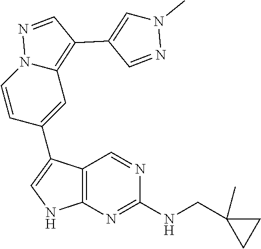 [SMILES: Cn1cc(-c2cnn3ccc(-c4c[nH]c5nc(NCC6(C)CC6)ncc45)cc23)cn1] | 1181 |
| 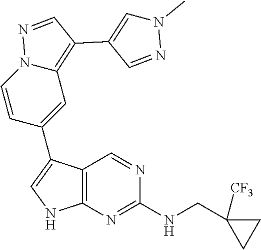 [SMILES: Cn1cc(-c2cnn3ccc(-c4c[nH]c5nc(NCC6(C(F)(F)F)CC6)ncc45)cc23)cn1] | 1182 |
| 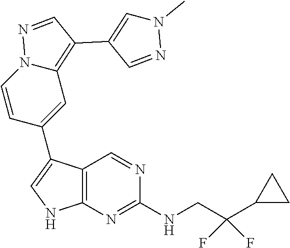 [SMILES: Cn1cc(-c2cnn3ccc(-c4c[nH]c5nc(NCC(F)(F)C6CC6)ncc45)cc23)cn1] | 1183 |
| 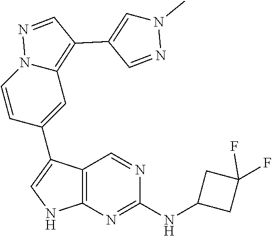 [SMILES: Cn1cc(-c2cnn3ccc(-c4c[nH]c5nc(NC6CC(F)(F)C6)ncc45)cc23)cn1] | 1184 |
| 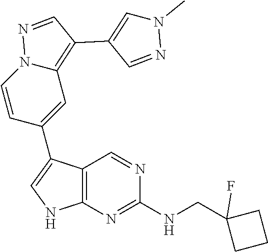 [SMILES: Cn1cc(-c2cnn3ccc(-c4c[nH]c5nc(NCC6(F)CCC6)ncc45)cc23)cn1] | 1185 |
| 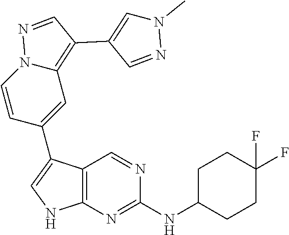 [SMILES: Cn1cc(-c2cnn3ccc(-c4c[nH]c5nc(NC6CCC(F)(F)CC6)ncc45)cc23)cn1] | 1186 |
| 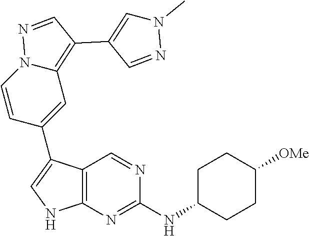 [SMILES: CO[C@H]1CC[C@@H](Nc2ncc3c(-c4ccn5ncc(-c6cnn(C)c6)c5c4)c[nH]c3n2)CC1] | 1187 |
| 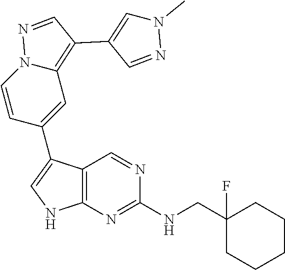 [SMILES: Cn1cc(-c2cnn3ccc(-c4c[nH]c5nc(NCC6(F)CCCCC6)ncc45)cc23)cn1] | 1188 |
| 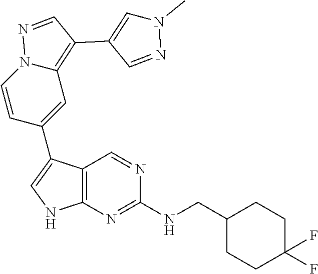 [SMILES: Cn1cc(-c2cnn3ccc(-c4c[nH]c5nc(NCC6CCC(F)(F)CC6)ncc45)cc23)cn1] | 1189 |
| 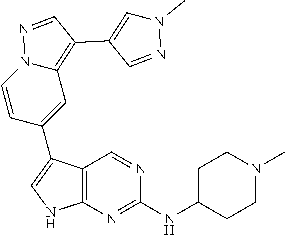 [SMILES: CN1CCC(Nc2ncc3c(-c4ccn5ncc(-c6cnn(C)c6)c5c4)c[nH]c3n2)CC1] | 1190 |
| 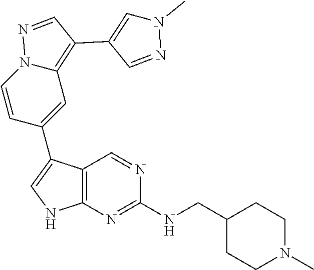 [SMILES: CN1CCC(CNc2ncc3c(-c4ccn5ncc(-c6cnn(C)c6)c5c4)c[nH]c3n2)CC1] | 1191 |
| 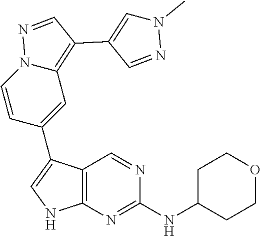 [SMILES: Cn1cc(-c2cnn3ccc(-c4c[nH]c5nc(NC6CCOCC6)ncc45)cc23)cn1] | 1192 |
| 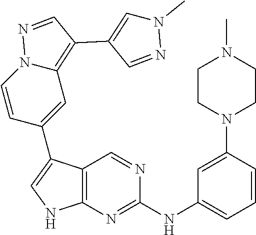 [SMILES: CN1CCN(c2cccc(Nc3ncc4c(-c5ccn6ncc(-c7cnn(C)c7)c6c5)c[nH]c4n3)c2)CC1] | 1193 |
| 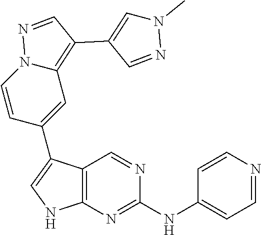 [SMILES: Cn1cc(-c2cnn3ccc(-c4c[nH]c5nc(Nc6ccncc6)ncc45)cc23)cn1] | 1194 |
| 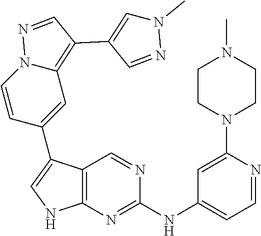 [SMILES: CN1CCN(c2cc(Nc3ncc4c(-c5ccn6ncc(-c7cnn(C)c7)c6c5)c[nH]c4n3)ccn2)CC1] | 1195 |
| 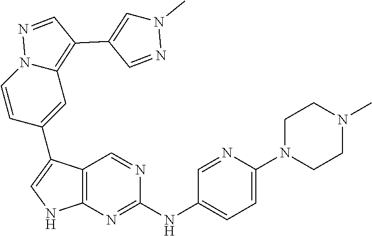 [SMILES: CN1CCN(c2ccc(Nc3ncc4c(-c5ccn6ncc(-c7cnn(C)c7)c6c5)c[nH]c4n3)cn2)CC1] | 1196 |
| 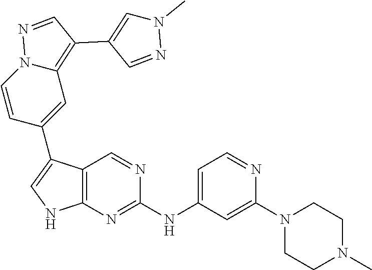 [SMILES: CN1CCN(c2cc(Nc3ncc4c(-c5ccn6ncc(-c7cnn(C)c7)c6c5)c[nH]c4n3)ccn2)CC1] | 1197 |
| 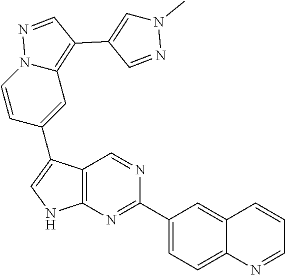 [SMILES: Cn1cc(-c2cnn3ccc(-c4c[nH]c5nc(-c6ccc7ncccc7c6)ncc45)cc23)cn1] | 1198 |
| 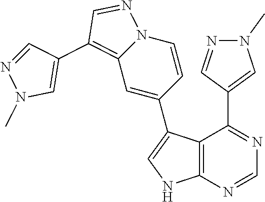 [SMILES: Cn1cc(-c2ncnc3[nH]cc(-c4ccn5ncc(-c6cnn(C)c6)c5c4)c23)cn1] | 1199 |
| 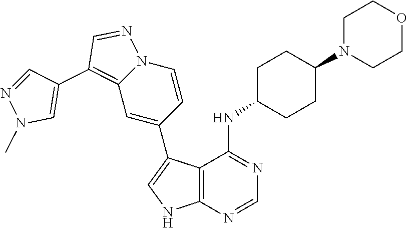 [SMILES: Cn1cc(-c2cnn3ccc(-c4c[nH]c5ncnc(N[C@H]6CC[C@H](N7CCOCC7)CC6)c45)cc23)cn1] | 1200 |
| 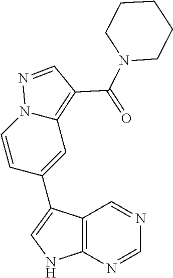 [SMILES: O=C(c1cnn2ccc(-c3c[nH]c4ncncc34)cc12)N1CCCCC1] | 1201 |
| 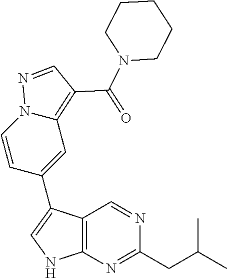 [SMILES: CC(C)Cc1ncc2c(-c3ccn4ncc(C(=O)N5CCCCC5)c4c3)c[nH]c2n1] | 1202 |
| 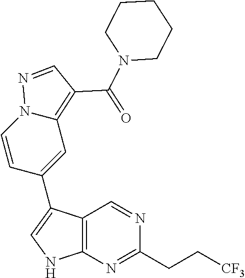 [SMILES: O=C(c1cnn2ccc(-c3c[nH]c4nc(CCC(F)(F)F)ncc34)cc12)N1CCCCC1] | 1203 |
| 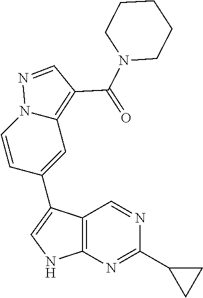 [SMILES: O=C(c1cnn2ccc(-c3c[nH]c4nc(C5CC5)ncc34)cc12)N1CCCCC1] | 1204 |
| 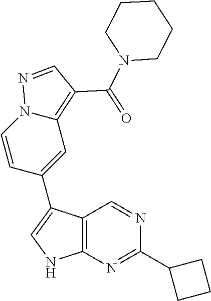 [SMILES: O=C(c1cnn2ccc(-c3c[nH]c4nc(C5CCC5)ncc34)cc12)N1CCCCC1] | 1205 |
| 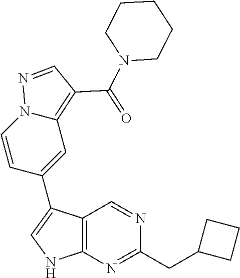 [SMILES: O=C(c1cnn2ccc(-c3c[nH]c4nc(CC5CCC5)ncc34)cc12)N1CCCCC1] | 1206 |
| 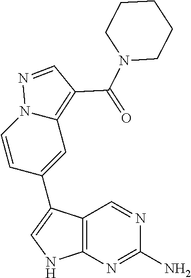 [SMILES: Nc1ncc2c(-c3ccn4ncc(C(=O)N5CCCCC5)c4c3)c[nH]c2n1] | 1207 |
| 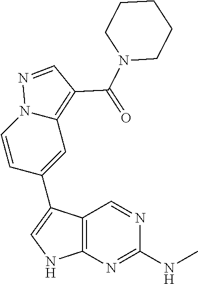 [SMILES: CNc1ncc2c(-c3ccn4ncc(C(=O)N5CCCCC5)c4c3)c[nH]c2n1] | 1208 |
| 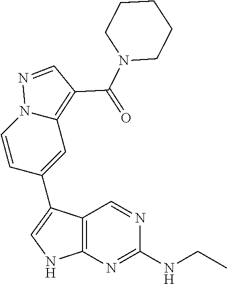 [SMILES: CCNc1ncc2c(-c3ccn4ncc(C(=O)N5CCCCC5)c4c3)c[nH]c2n1] | 1209 |
| 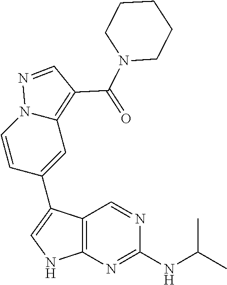 [SMILES: CC(C)Nc1ncc2c(-c3ccn4ncc(C(=O)N5CCCCC5)c4c3)c[nH]c2n1] | 1210 |
| 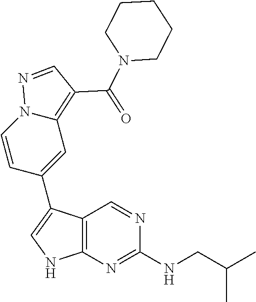 [SMILES: CC(C)CNc1ncc2c(-c3ccn4ncc(C(=O)N5CCCCC5)c4c3)c[nH]c2n1] | 1211 |
| 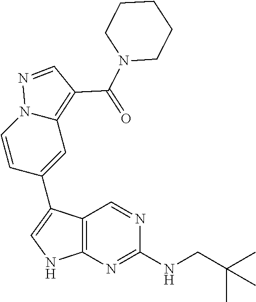 [SMILES: CC(C)(C)CNc1ncc2c(-c3ccn4ncc(C(=O)N5CCCCC5)c4c3)c[nH]c2n1] | 1212 |
| 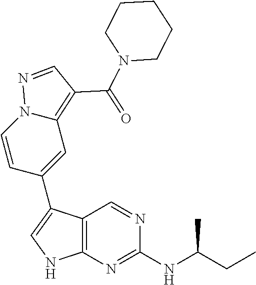 [SMILES: CC[C@H](C)Nc1ncc2c(-c3ccn4ncc(C(=O)N5CCCCC5)c4c3)c[nH]c2n1] | 1213 |
| 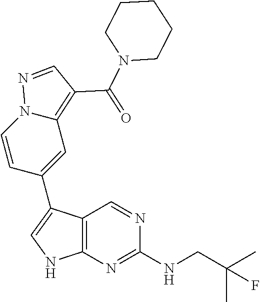 [SMILES: CC(C)(F)CNc1ncc2c(-c3ccn4ncc(C(=O)N5CCCCC5)c4c3)c[nH]c2n1] | 1214 |
| 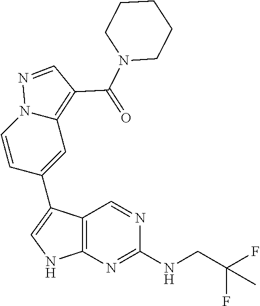 [SMILES: CC(F)(F)CNc1ncc2c(-c3ccn4ncc(C(=O)N5CCCCC5)c4c3)c[nH]c2n1] | 1215 |
| 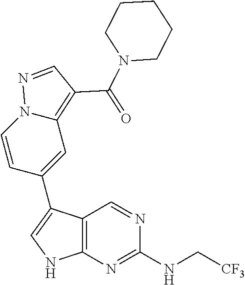 [SMILES: O=C(c1cnn2ccc(-c3c[nH]c4nc(NCC(F)(F)F)ncc34)cc12)N1CCCCC1] | 1216 |
| 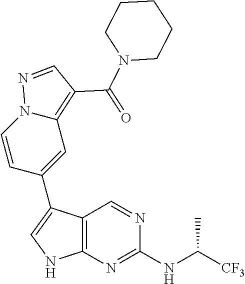 [SMILES: C[C@@H](Nc1ncc2c(-c3ccn4ncc(C(=O)N5CCCCC5)c4c3)c[nH]c2n1)C(F)(F)F] | 1217 |
| 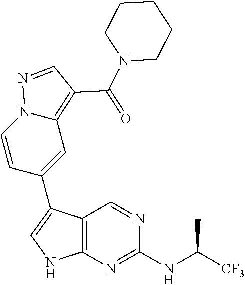 [SMILES: C[C@H](Nc1ncc2c(-c3ccn4ncc(C(=O)N5CCCCC5)c4c3)c[nH]c2n1)C(F)(F)F] | 1218 |
| 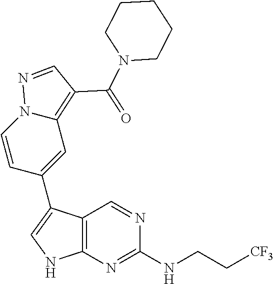 [SMILES: O=C(c1cnn2ccc(-c3c[nH]c4nc(NCCC(F)(F)F)ncc34)cc12)N1CCCCC1] | 1219 |
| 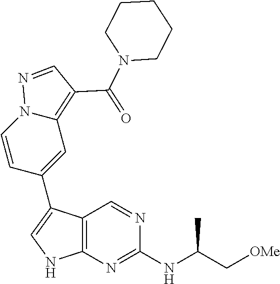 [SMILES: COC[C@H](C)Nc1ncc2c(-c3ccn4ncc(C(=O)N5CCCCC5)c4c3)c[nH]c2n1] | 1220 |
| 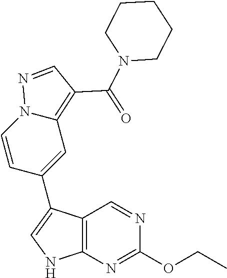 [SMILES: CCOc1ncc2c(-c3ccn4ncc(C(=O)N5CCCCC5)c4c3)c[nH]c2n1] | 1221 |
| 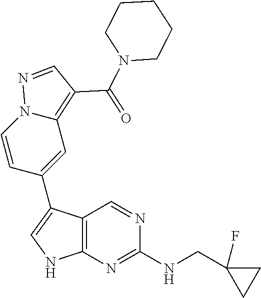 [SMILES: O=C(c1cnn2ccc(-c3c[nH]c4nc(NCC5(F)CC5)ncc34)cc12)N1CCCCC1] | 1222 |
| 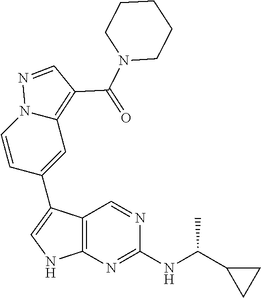 [SMILES: C[C@@H](Nc1ncc2c(-c3ccn4ncc(C(=O)N5CCCCC5)c4c3)c[nH]c2n1)C1CC1] | 1223 |
| 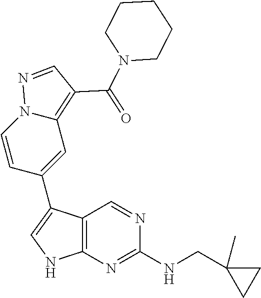 [SMILES: CC1(CNc2ncc3c(-c4ccn5ncc(C(=O)N6CCCCC6)c5c4)c[nH]c3n2)CC1] | 1224 |
| 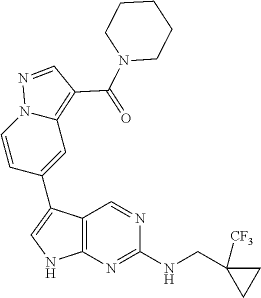 [SMILES: O=C(c1cnn2ccc(-c3c[nH]c4nc(NCC5(C(F)(F)F)CC5)ncc34)cc12)N1CCCCC1] | 1225 |
| 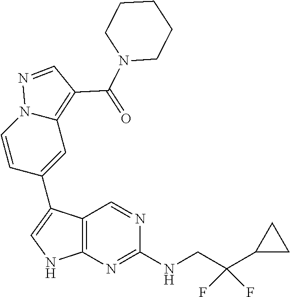 [SMILES: O=C(c1cnn2ccc(-c3c[nH]c4nc(NCC(F)(F)C5CC5)ncc34)cc12)N1CCCCC1] | 1226 |
| 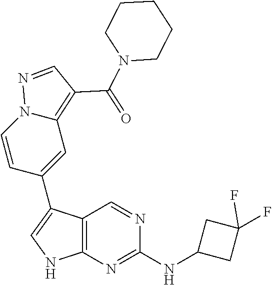 [SMILES: O=C(c1cnn2ccc(-c3c[nH]c4nc(NC5CC(F)(F)C5)ncc34)cc12)N1CCCCC1] | 1227 |
| 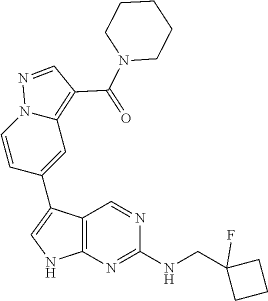 [SMILES: O=C(c1cnn2ccc(-c3c[nH]c4nc(NCC5(F)CCC5)ncc34)cc12)N1CCCCC1] | 1228 |
| 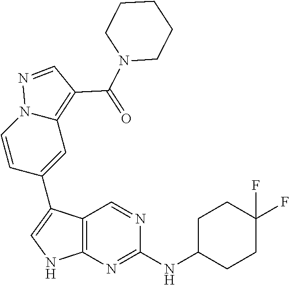 [SMILES: O=C(c1cnn2ccc(-c3c[nH]c4nc(NC5CCC(F)(F)CC5)ncc34)cc12)N1CCCCC1] | 1229 |
| 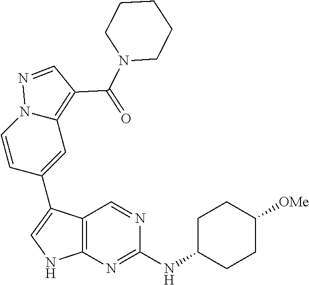 [SMILES: CO[C@H]1CC[C@@H](Nc2ncc3c(-c4ccn5ncc(C(=O)N6CCCCC6)c5c4)c[nH]c3n2)CC1] | 1230 |
| 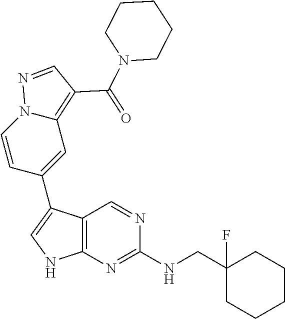 [SMILES: O=C(c1cnn2ccc(-c3c[nH]c4nc(NCC5(F)CCCCC5)ncc34)cc12)N1CCCCC1] | 1231 |
| 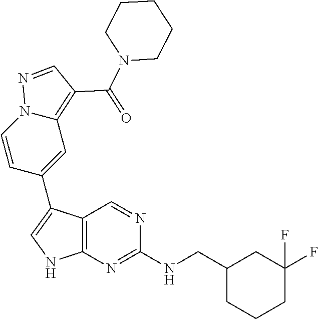 [SMILES: O=C(c1cnn2ccc(-c3c[nH]c4nc(NCC5CCCC(F)(F)C5)ncc34)cc12)N1CCCCC1] | 1232 |
| 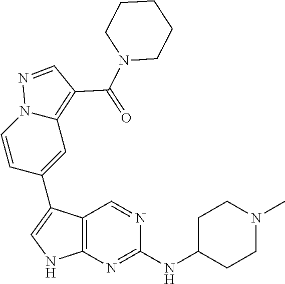 [SMILES: CN1CCC(Nc2ncc3c(-c4ccn5ncc(C(=O)N6CCCCC6)c5c4)c[nH]c3n2)CC1] | 1233 |
| 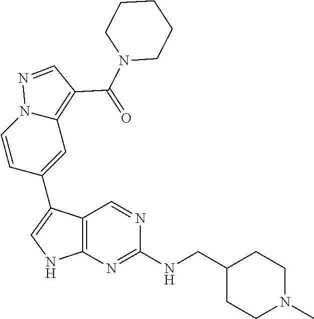 [SMILES: CN1CCC(CNc2ncc3c(-c4ccn5ncc(C(=O)N6CCCCC6)c5c4)c[nH]c3n2)CC1] | 1234 |
| 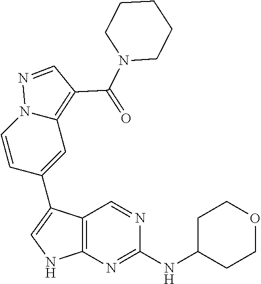 [SMILES: O=C(c1cnn2ccc(-c3c[nH]c4nc(NC5CCOCC5)ncc34)cc12)N1CCCCC1] | 1235 |
| 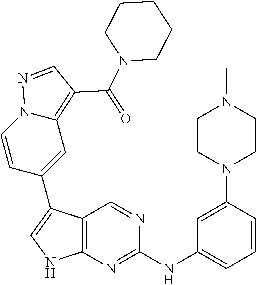 [SMILES: CN1CCN(c2cccc(Nc3ncc4c(-c5ccn6ncc(C(=O)N7CCCCC7)c6c5)c[nH]c4n3)c2)CC1] | 1236 |
| 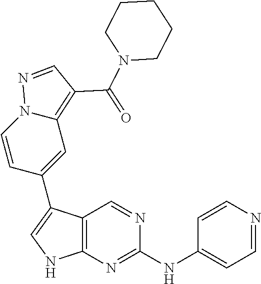 [SMILES: O=C(c1cnn2ccc(-c3c[nH]c4nc(Nc5ccncc5)ncc34)cc12)N1CCCCC1] | 1237 |
| 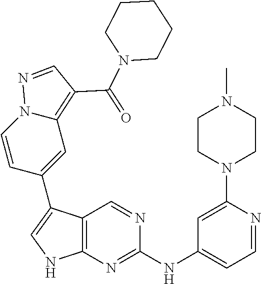 [SMILES: CN1CCN(c2cc(Nc3ncc4c(-c5ccn6ncc(C(=O)N7CCCCC7)c6c5)c[nH]c4n3)ccn2)CC1] | 1238 |
| 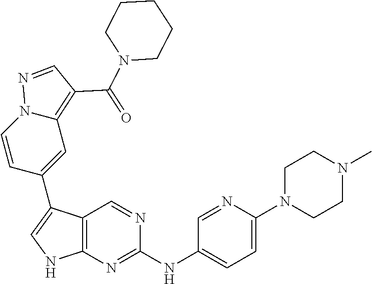 [SMILES: CN1CCN(c2ccc(Nc3ncc4c(-c5ccn6ncc(C(=O)N7CCCCC7)c6c5)c[nH]c4n3)cn2)CC1] | 1239 |
| 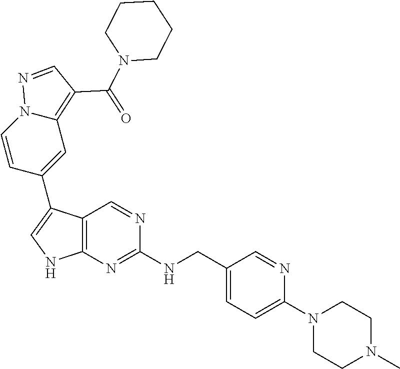 [SMILES: CN1CCN(c2ccc(CNc3ncc4c(-c5ccn6ncc(C(=O)N7CCCCC7)c6c5)c[nH]c4n3)cn2)CC1] | 1240 |
| 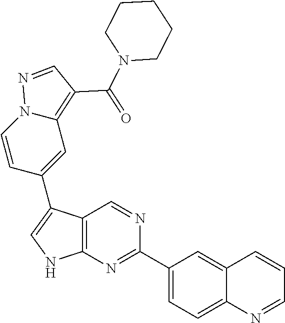 [SMILES: O=C(c1cnn2ccc(-c3c[nH]c4nc(-c5ccc6ncccc6c5)ncc34)cc12)N1CCCCC1] | 1241 |
| 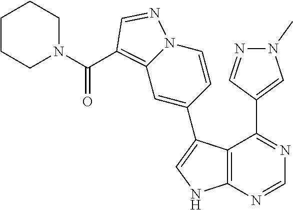 [SMILES: Cn1cc(-c2ncnc3[nH]cc(-c4ccn5ncc(C(=O)N6CCCCC6)c5c4)c23)cn1] | 1242 |
| 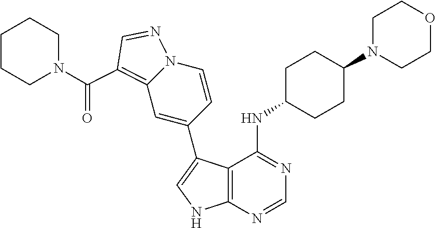 [SMILES: O=C(c1cnn2ccc(-c3c[nH]c4ncnc(N[C@H]5CC[C@H](N6CCOCC6)CC5)c34)cc12)N1CCCCC1] | 1243 |
| 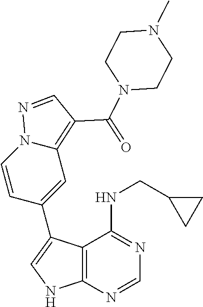 [SMILES: CN1CCN(C(=O)c2cnn3ccc(-c4c[nH]c5ncnc(NCC6CC6)c45)cc23)CC1] | 1244 |
| 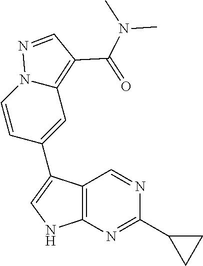 [SMILES: CN(C)C(=O)c1cnn2ccc(-c3c[nH]c4nc(C5CC5)ncc34)cc12] | 1245 |
| 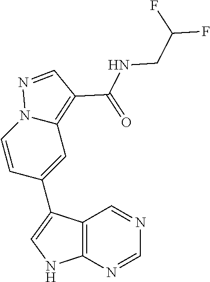 [SMILES: O=C(NCC(F)F)c1cnn2ccc(-c3c[nH]c4ncncc34)cc12] | 1246 |
| 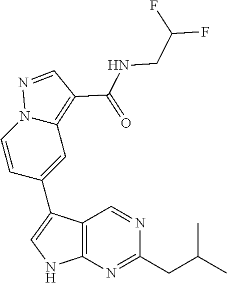 [SMILES: CC(C)Cc1ncc2c(-c3ccn4ncc(C(=O)NCC(F)F)c4c3)c[nH]c2n1] | 1247 |
| 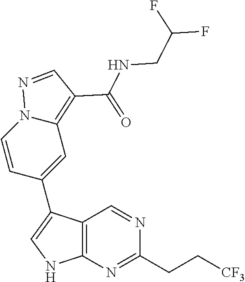 [SMILES: O=C(NCC(F)F)c1cnn2ccc(-c3c[nH]c4nc(CCC(F)(F)F)ncc34)cc12] | 1248 |
| 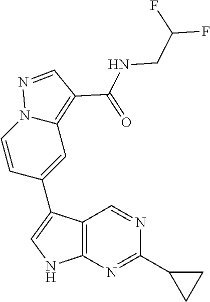 [SMILES: O=C(NCC(F)F)c1cnn2ccc(-c3c[nH]c4nc(C5CC5)ncc34)cc12] | 1249 |
| 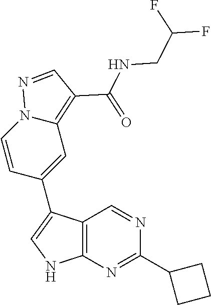 [SMILES: O=C(NCC(F)F)c1cnn2ccc(-c3c[nH]c4nc(C5CCC5)ncc34)cc12] | 1250 |
| 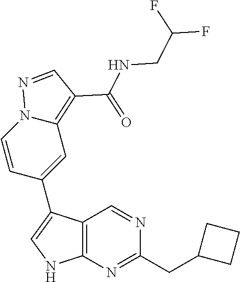 [SMILES: O=C(NCC(F)F)c1cnn2ccc(-c3c[nH]c4nc(CC5CCC5)ncc34)cc12] | 1251 |
| 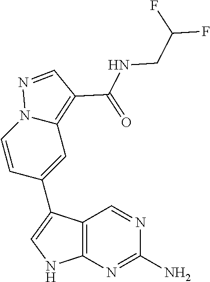 [SMILES: Nc1ncc2c(-c3ccn4ncc(C(=O)NCC(F)F)c4c3)c[nH]c2n1] | 1252 |
| 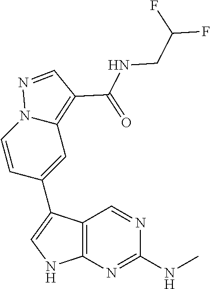 [SMILES: CNc1ncc2c(-c3ccn4ncc(C(=O)NCC(F)F)c4c3)c[nH]c2n1] | 1253 |
| 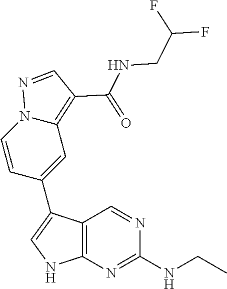 [SMILES: CCNc1ncc2c(-c3ccn4ncc(C(=O)NCC(F)F)c4c3)c[nH]c2n1] | 1254 |
| 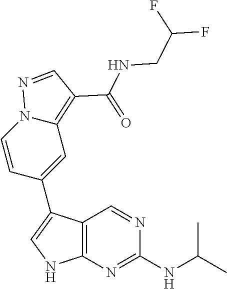 [SMILES: CC(C)Nc1ncc2c(-c3ccn4ncc(C(=O)NCC(F)F)c4c3)c[nH]c2n1] | 1255 |
| 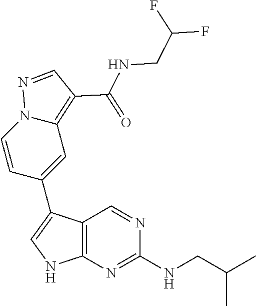 [SMILES: CC(C)CNc1ncc2c(-c3ccn4ncc(C(=O)NCC(F)F)c4c3)c[nH]c2n1] | 1256 |
| 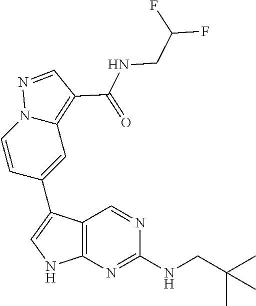 [SMILES: CC(C)(C)CNc1ncc2c(-c3ccn4ncc(C(=O)NCC(F)F)c4c3)c[nH]c2n1] | 1257 |
| 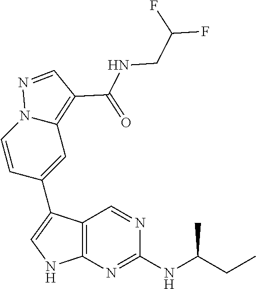 [SMILES: CC[C@H](C)Nc1ncc2c(-c3ccn4ncc(C(=O)NCC(F)F)c4c3)c[nH]c2n1] | 1258 |
| 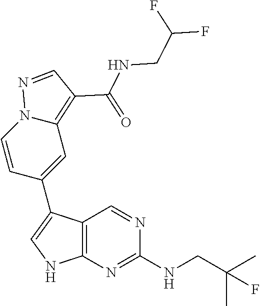 [SMILES: CC(C)(F)CNc1ncc2c(-c3ccn4ncc(C(=O)NCC(F)F)c4c3)c[nH]c2n1] | 1259 |
| 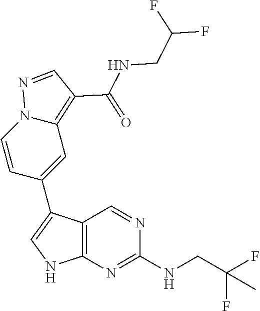 [SMILES: CC(F)(F)CNc1ncc2c(-c3ccn4ncc(C(=O)NCC(F)F)c4c3)c[nH]c2n1] | 1260 |
| 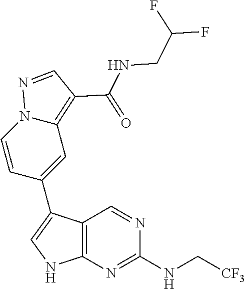 [SMILES: O=C(NCC(F)F)c1cnn2ccc(-c3c[nH]c4nc(NCC(F)(F)F)ncc34)cc12] | 1261 |
| 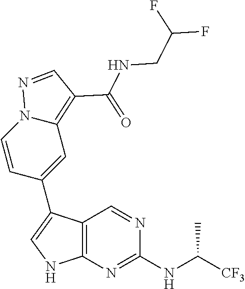 [SMILES: C[C@@H](Nc1ncc2c(-c3ccn4ncc(C(=O)NCC(F)F)c4c3)c[nH]c2n1)C(F)(F)F] | 1262 |
| 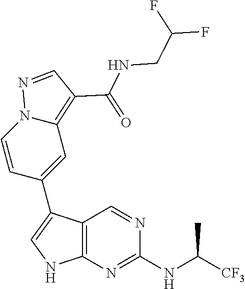 [SMILES: C[C@H](Nc1ncc2c(-c3ccn4ncc(C(=O)NCC(F)F)c4c3)c[nH]c2n1)C(F)(F)F] | 1263 |
| 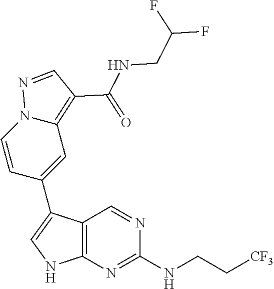 [SMILES: O=C(NCC(F)F)c1cnn2ccc(-c3c[nH]c4nc(NCCC(F)(F)F)ncc34)cc12] | 1264 |
| 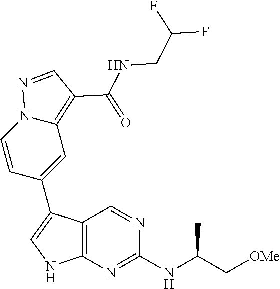 [SMILES: COC[C@H](C)Nc1ncc2c(-c3ccn4ncc(C(=O)NCC(F)F)c4c3)c[nH]c2n1] | 1265 |
| 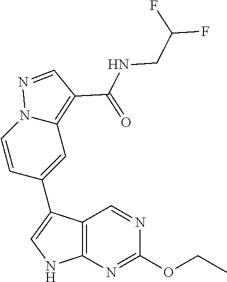 [SMILES: CCOc1ncc2c(-c3ccn4ncc(C(=O)NCC(F)F)c4c3)c[nH]c2n1] | 1266 |
| 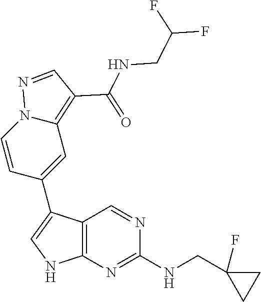 [SMILES: O=C(NCC(F)F)c1cnn2ccc(-c3c[nH]c4nc(NCC5(F)CC5)ncc34)cc12] | 1267 |
| 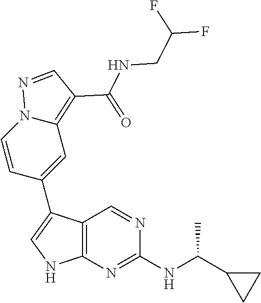 [SMILES: C[C@@H](Nc1ncc2c(-c3ccn4ncc(C(=O)NCC(F)F)c4c3)c[nH]c2n1)C1CC1] | 1268 |
| 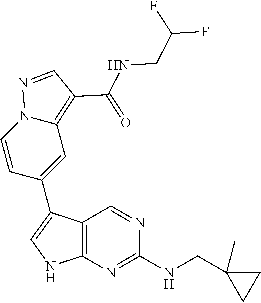 [SMILES: CC1(CNc2ncc3c(-c4ccn5ncc(C(=O)NCC(F)F)c5c4)c[nH]c3n2)CC1] | 1269 |
| 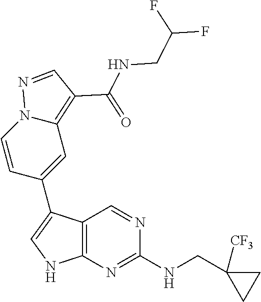 [SMILES: O=C(NCC(F)F)c1cnn2ccc(-c3c[nH]c4nc(NCC5(C(F)(F)F)CC5)ncc34)cc12] | 1270 |
| 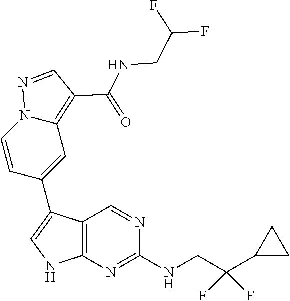 [SMILES: O=C(NCC(F)F)c1cnn2ccc(-c3c[nH]c4nc(NCC(F)(F)C5CC5)ncc34)cc12] | 1271 |
| 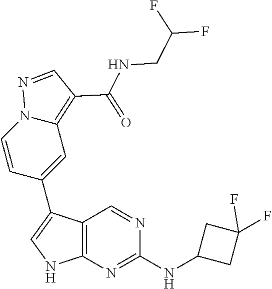 [SMILES: O=C(NCC(F)F)c1cnn2ccc(-c3c[nH]c4nc(NC5CC(F)(F)C5)ncc34)cc12] | 1272 |
| 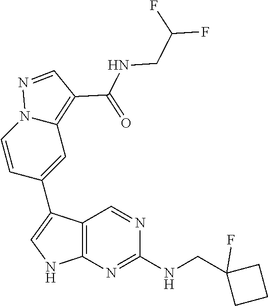 [SMILES: O=C(NCC(F)F)c1cnn2ccc(-c3c[nH]c4nc(NCC5(F)CCC5)ncc34)cc12] | 1273 |
| 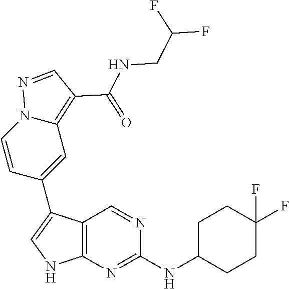 [SMILES: O=C(NCC(F)F)c1cnn2ccc(-c3c[nH]c4nc(NC5CCC(F)(F)CC5)ncc34)cc12] | 1274 |
| 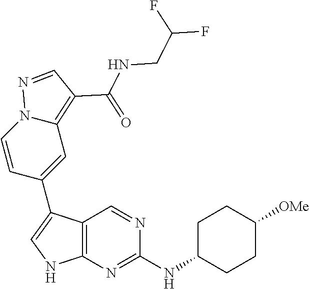 [SMILES: CO[C@H]1CC[C@@H](Nc2ncc3c(-c4ccn5ncc(C(=O)NCC(F)F)c5c4)c[nH]c3n2)CC1] | 1275 |
| 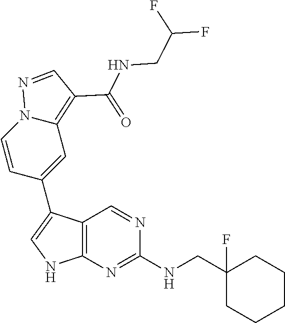 [SMILES: O=C(NCC(F)F)c1cnn2ccc(-c3c[nH]c4nc(NCC5(F)CCCCC5)ncc34)cc12] | 1276 |
| 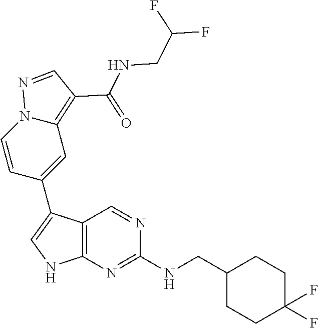 [SMILES: O=C(NCC(F)F)c1cnn2ccc(-c3c[nH]c4nc(NCC5CCC(F)(F)CC5)ncc34)cc12] | 1277 |
| 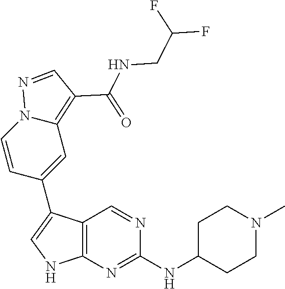 [SMILES: CN1CCC(Nc2ncc3c(-c4ccn5ncc(C(=O)NCC(F)F)c5c4)c[nH]c3n2)CC1] | 1278 |
| 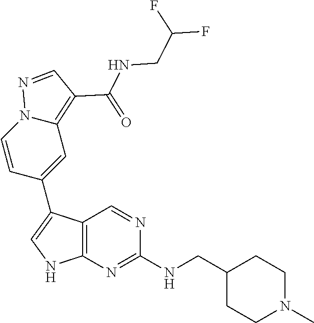 [SMILES: CN1CCC(CNc2ncc3c(-c4ccn5ncc(C(=O)NCC(F)F)c5c4)c[nH]c3n2)CC1] | 1279 |
| 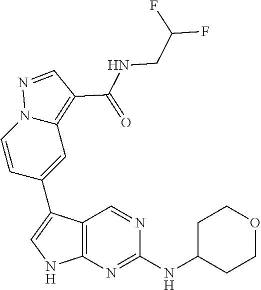 [SMILES: O=C(NCC(F)F)c1cnn2ccc(-c3c[nH]c4nc(NC5CCOCC5)ncc34)cc12] | 1280 |
| 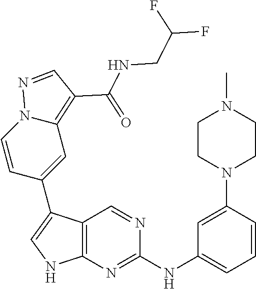 [SMILES: CN1CCN(c2cccc(Nc3ncc4c(-c5ccn6ncc(C(=O)NCC(F)F)c6c5)c[nH]c4n3)c2)CC1] | 1281 |
| 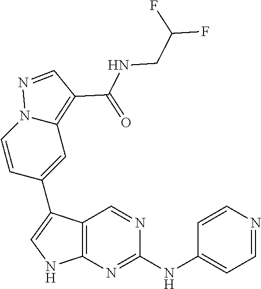 [SMILES: O=C(NCC(F)F)c1cnn2ccc(-c3c[nH]c4nc(Nc5ccncc5)ncc34)cc12] | 1282 |
| 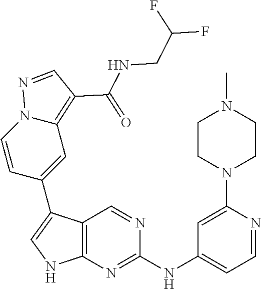 [SMILES: CN1CCN(c2cc(Nc3ncc4c(-c5ccn6ncc(C(=O)NCC(F)F)c6c5)c[nH]c4n3)ccn2)CC1] | 1283 |
| 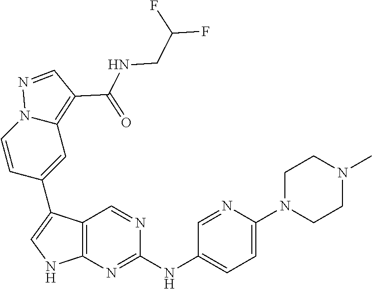 [SMILES: CN1CCN(c2ccc(Nc3ncc4c(-c5ccn6ncc(C(=O)NCC(F)F)c6c5)c[nH]c4n3)cn2)CC1] | 1284 |
| 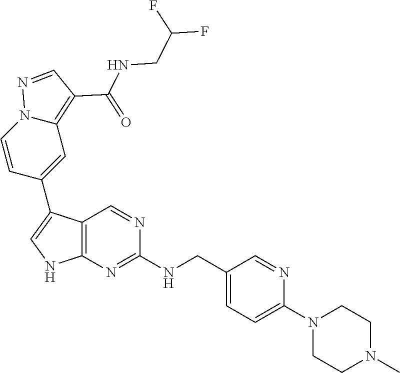 [SMILES: CN1CCN(c2ccc(CNc3ncc4c(-c5ccn6ncc(C(=O)NCC(F)F)c6c5)c[nH]c4n3)cn2)CC1] | 1285 |
| 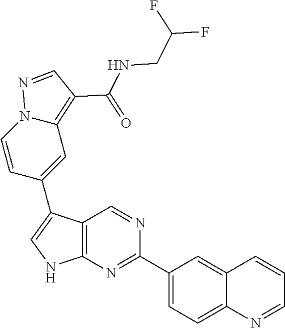 [SMILES: O=C(NCC(F)F)c1cnn2ccc(-c3c[nH]c4nc(-c5ccc6ncccc6c5)ncc34)cc12] | 1286 |
| 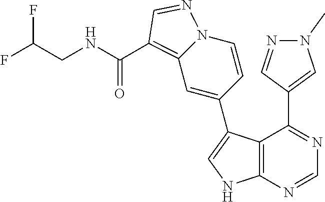 [SMILES: Cn1cc(-c2ncnc3[nH]cc(-c4ccn5ncc(C(=O)NCC(F)F)c5c4)c23)cn1] | 1287 |
| 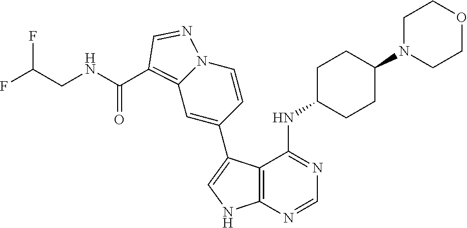 [SMILES: O=C(NCC(F)F)c1cnn2ccc(-c3c[nH]c4ncnc(N[C@H]5CC[C@H](N6CCOCC6)CC5)c34)cc12] | 1288 |
| 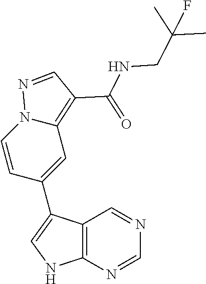 [SMILES: CC(C)(F)CNC(=O)c1cnn2ccc(-c3c[nH]c4ncncc34)cc12] | 1289 |
| 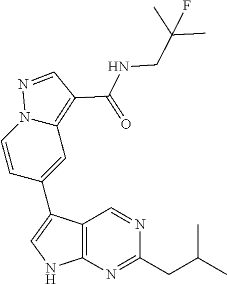 [SMILES: CC(C)Cc1ncc2c(-c3ccn4ncc(C(=O)NCC(C)(C)F)c4c3)c[nH]c2n1] | 1290 |
| 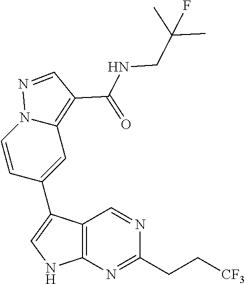 [SMILES: CC(C)(F)CNC(=O)c1cnn2ccc(-c3c[nH]c4nc(CCC(F)(F)F)ncc34)cc12] | 1291 |
| 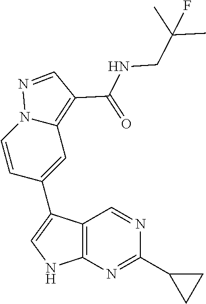 [SMILES: CC(C)(F)CNC(=O)c1cnn2ccc(-c3c[nH]c4nc(C5CC5)ncc34)cc12] | 1292 |
| 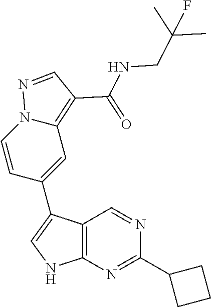 [SMILES: CC(C)(F)CNC(=O)c1cnn2ccc(-c3c[nH]c4nc(C5CCC5)ncc34)cc12] | 1293 |
| 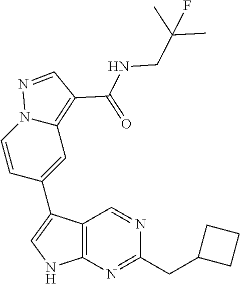 [SMILES: CC(C)(F)CNC(=O)c1cnn2ccc(-c3c[nH]c4nc(CC5CCC5)ncc34)cc12] | 1294 |
| 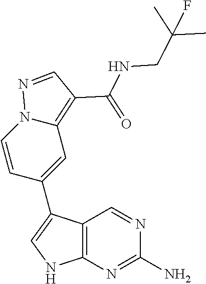 [SMILES: CC(C)(F)CNC(=O)c1cnn2ccc(-c3c[nH]c4nc(N)ncc34)cc12] | 1295 |
| 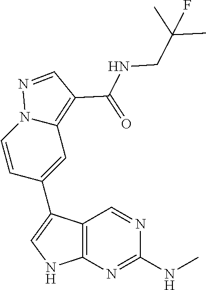 [SMILES: CNc1ncc2c(-c3ccn4ncc(C(=O)NCC(C)(C)F)c4c3)c[nH]c2n1] | 1296 |
| 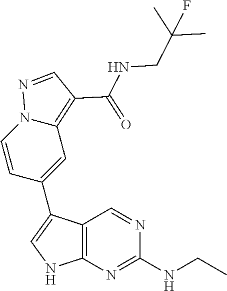 [SMILES: CCNc1ncc2c(-c3ccn4ncc(C(=O)NCC(C)(C)F)c4c3)c[nH]c2n1] | 1297 |
| 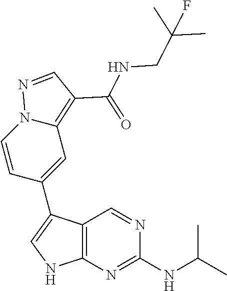 [SMILES: CC(C)Nc1ncc2c(-c3ccn4ncc(C(=O)NCC(C)(C)F)c4c3)c[nH]c2n1] | 1298 |
| 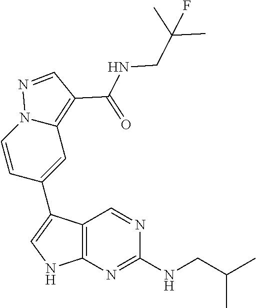 [SMILES: CC(C)CNc1ncc2c(-c3ccn4ncc(C(=O)NCC(C)(C)F)c4c3)c[nH]c2n1] | 1299 |
| 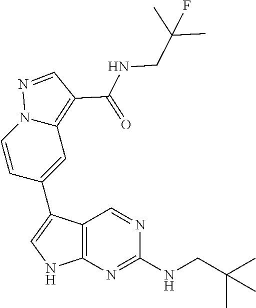 [SMILES: CC(C)(C)CNc1ncc2c(-c3ccn4ncc(C(=O)NCC(C)(C)F)c4c3)c[nH]c2n1] | 1300 |
| 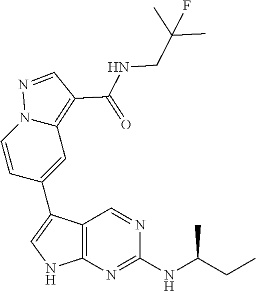 [SMILES: CC[C@H](C)Nc1ncc2c(-c3ccn4ncc(C(=O)NCC(C)(C)F)c4c3)c[nH]c2n1] | 1301 |
| 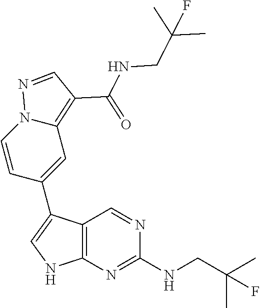 [SMILES: CC(C)(F)CNC(=O)c1cnn2ccc(-c3c[nH]c4nc(NCC(C)(C)F)ncc34)cc12] | 1302 |
| 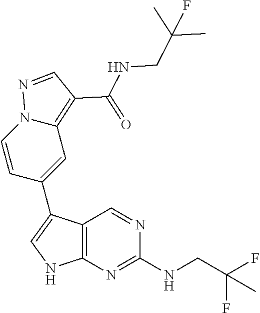 [SMILES: CC(C)(F)CNC(=O)c1cnn2ccc(-c3c[nH]c4nc(NCC(C)(F)F)ncc34)cc12] | 1303 |
| 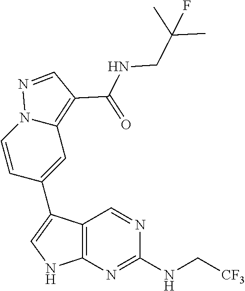 [SMILES: CC(C)(F)CNC(=O)c1cnn2ccc(-c3c[nH]c4nc(NCC(F)(F)F)ncc34)cc12] | 1304 |
| 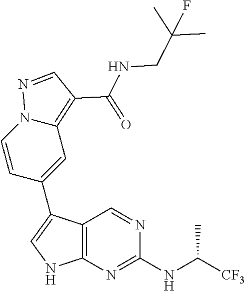 [SMILES: C[C@@H](Nc1ncc2c(-c3ccn4ncc(C(=O)NCC(C)(C)F)c4c3)c[nH]c2n1)C(F)(F)F] | 1305 |
| 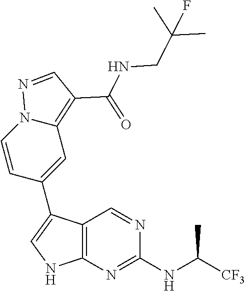 [SMILES: C[C@H](Nc1ncc2c(-c3ccn4ncc(C(=O)NCC(C)(C)F)c4c3)c[nH]c2n1)C(F)(F)F] | 1306 |
| 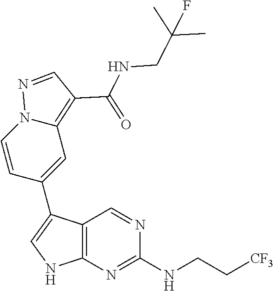 [SMILES: CC(C)(F)CNC(=O)c1cnn2ccc(-c3c[nH]c4nc(NCCC(F)(F)F)ncc34)cc12] | 1307 |
| 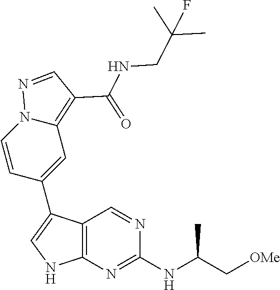 [SMILES: COC[C@H](C)Nc1ncc2c(-c3ccn4ncc(C(=O)NCC(C)(C)F)c4c3)c[nH]c2n1] | 1308 |
| 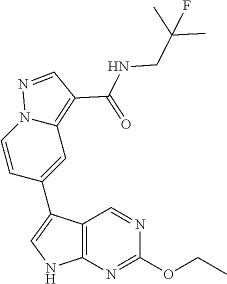 [SMILES: CCOc1ncc2c(-c3ccn4ncc(C(=O)NCC(C)(C)F)c4c3)c[nH]c2n1] | 1309 |
| 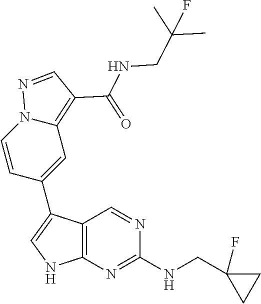 [SMILES: CC(C)(F)CNC(=O)c1cnn2ccc(-c3c[nH]c4nc(NCC5(F)CC5)ncc34)cc12] | 1310 |
| 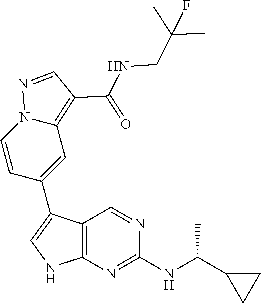 [SMILES: C[C@@H](Nc1ncc2c(-c3ccn4ncc(C(=O)NCC(C)(C)F)c4c3)c[nH]c2n1)C1CC1] | 1311 |
| 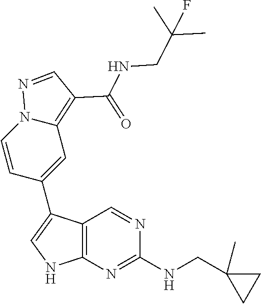 [SMILES: CC(C)(F)CNC(=O)c1cnn2ccc(-c3c[nH]c4nc(NCC5(C)CC5)ncc34)cc12] | 1312 |
| 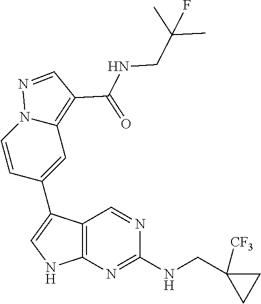 [SMILES: CC(C)(F)CNC(=O)c1cnn2ccc(-c3c[nH]c4nc(NCC5(C(F)(F)F)CC5)ncc34)cc12] | 1313 |
| 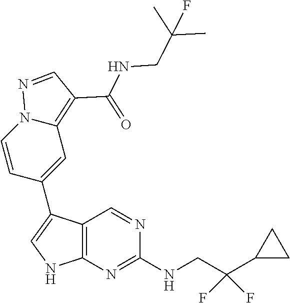 [SMILES: CC(C)(F)CNC(=O)c1cnn2ccc(-c3c[nH]c4nc(NCC(F)(F)C5CC5)ncc34)cc12] | 1314 |
| 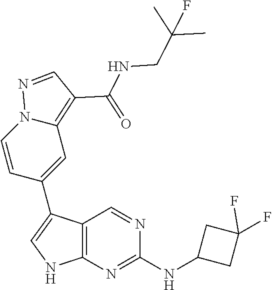 [SMILES: CC(C)(F)CNC(=O)c1cnn2ccc(-c3c[nH]c4nc(NC5CC(F)(F)C5)ncc34)cc12] | 1315 |
| 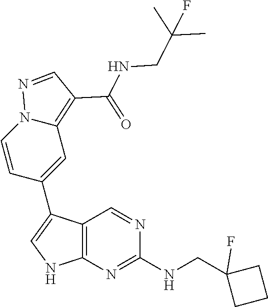 [SMILES: CC(C)(F)CNC(=O)c1cnn2ccc(-c3c[nH]c4nc(NCC5(F)CCC5)ncc34)cc12] | 1316 |
| 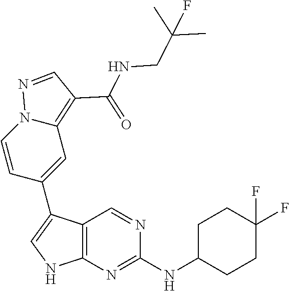 [SMILES: CC(C)(F)CNC(=O)c1cnn2ccc(-c3c[nH]c4nc(NC5CCC(F)(F)CC5)ncc34)cc12] | 1317 |
| 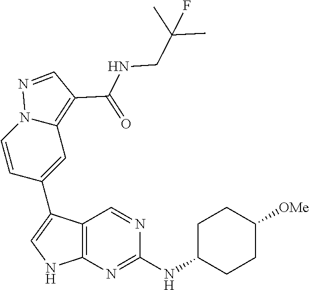 [SMILES: CO[C@H]1CC[C@@H](Nc2ncc3c(-c4ccn5ncc(C(=O)NCC(C)(C)F)c5c4)c[nH]c3n2)CC1] | 1318 |
| 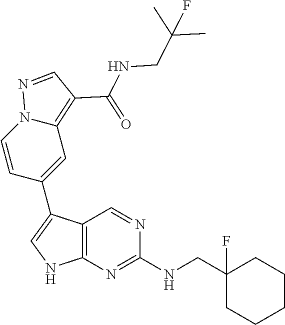 [SMILES: CC(C)(F)CNC(=O)c1cnn2ccc(-c3c[nH]c4nc(NCC5(F)CCCCC5)ncc34)cc12] | 1319 |
| 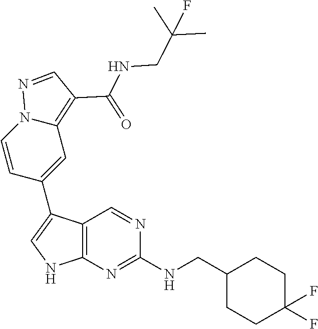 [SMILES: CC(C)(F)CNC(=O)c1cnn2ccc(-c3c[nH]c4nc(NCC5CCC(F)(F)CC5)ncc34)cc12] | 1320 |
| 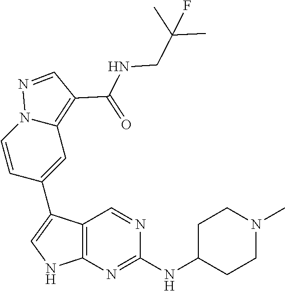 [SMILES: CN1CCC(Nc2ncc3c(-c4ccn5ncc(C(=O)NCC(C)(C)F)c5c4)c[nH]c3n2)CC1] | 1321 |
| 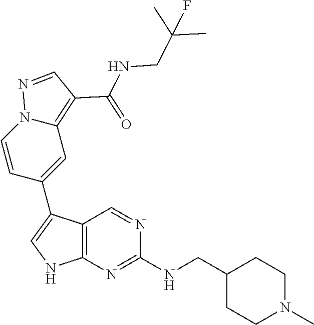 [SMILES: CN1CCC(CNc2ncc3c(-c4ccn5ncc(C(=O)NCC(C)(C)F)c5c4)c[nH]c3n2)CC1] | 1322 |
| 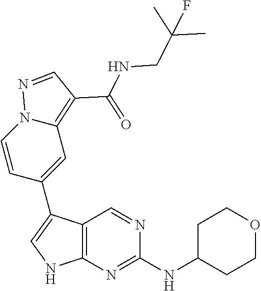 [SMILES: CC(C)(F)CNC(=O)c1cnn2ccc(-c3c[nH]c4nc(NC5CCOCC5)ncc34)cc12] | 1323 |
| 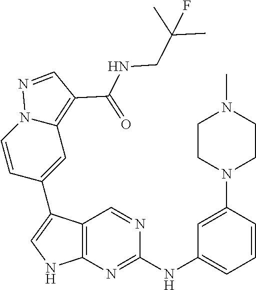 [SMILES: CN1CCN(c2cccc(Nc3ncc4c(-c5ccn6ncc(C(=O)NCC(C)(C)F)c6c5)c[nH]c4n3)c2)CC1] | 1324 |
| 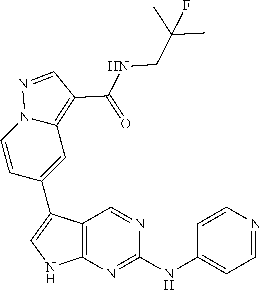 [SMILES: CC(C)(F)CNC(=O)c1cnn2ccc(-c3c[nH]c4nc(Nc5ccncc5)ncc34)cc12] | 1325 |
| 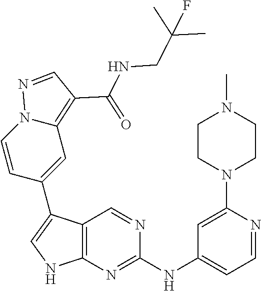 [SMILES: CN1CCN(c2cc(Nc3ncc4c(-c5ccn6ncc(C(=O)NCC(C)(C)F)c6c5)c[nH]c4n3)ccn2)CC1] | 1326 |
| 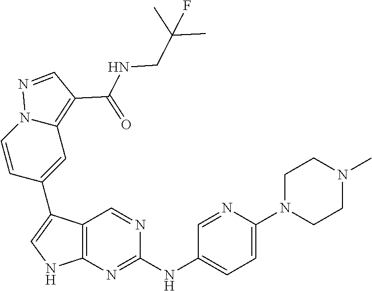 [SMILES: CN1CCN(c2ccc(Nc3ncc4c(-c5ccn6ncc(C(=O)NCC(C)(C)F)c6c5)c[nH]c4n3)cn2)CC1] | 1327 |
| 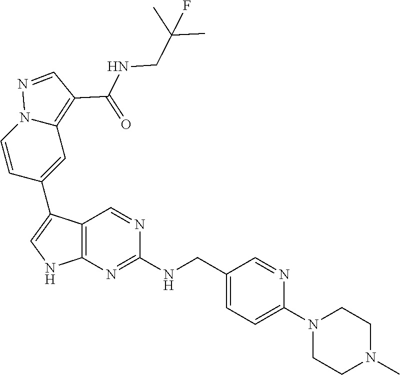 [SMILES: CN1CCN(c2ccc(CNc3ncc4c(-c5ccn6ncc(C(=O)NCC(C)(C)F)c6c5)c[nH]c4n3)cn2)CC1] | 1328 |
| 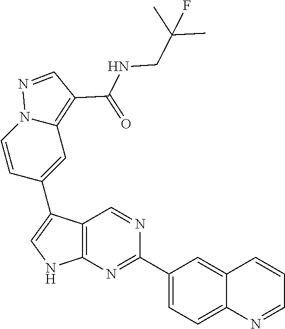 [SMILES: CC(C)(F)CNC(=O)c1cnn2ccc(-c3c[nH]c4nc(-c5ccc6ncccc6c5)ncc34)cc12] | 1329 |
| 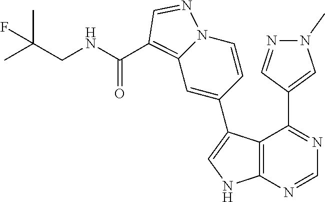 [SMILES: Cn1cc(-c2ncnc3[nH]cc(-c4ccn5ncc(C(=O)NCC(C)(C)F)c5c4)c23)cn1] | 1330 |
| 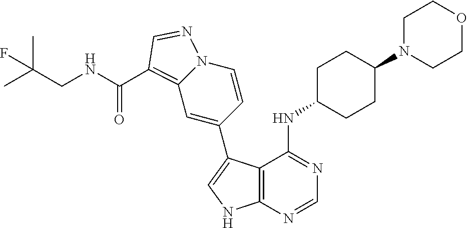 [SMILES: CC(C)(F)CNC(=O)c1cnn2ccc(-c3c[nH]c4ncnc(N[C@H]5CC[C@H](N6CCOCC6)CC5)c34)cc12] | 1331 |
| 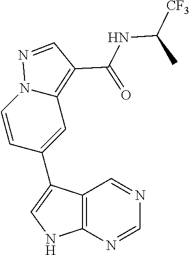 [SMILES: C[C@@H](NC(=O)c1cnn2ccc(-c3c[nH]c4ncncc34)cc12)C(F)(F)F] | 1332 |
| 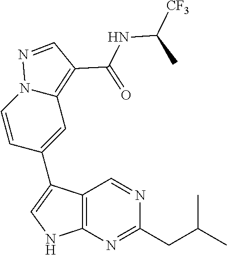 [SMILES: CC(C)Cc1ncc2c(-c3ccn4ncc(C(=O)N[C@H](C)C(F)(F)F)c4c3)c[nH]c2n1] | 1333 |
| 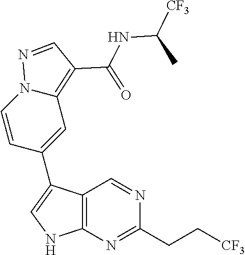 [SMILES: C[C@@H](NC(=O)c1cnn2ccc(-c3c[nH]c4nc(CCC(F)(F)F)ncc34)cc12)C(F)(F)F] | 1334 |
| 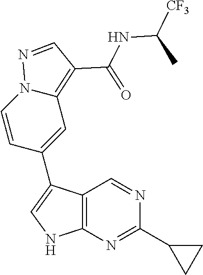 [SMILES: C[C@@H](NC(=O)c1cnn2ccc(-c3c[nH]c4nc(C5CC5)ncc34)cc12)C(F)(F)F] | 1335 |
| 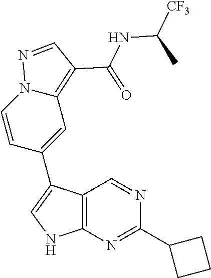 [SMILES: C[C@@H](NC(=O)c1cnn2ccc(-c3c[nH]c4nc(C5CCC5)ncc34)cc12)C(F)(F)F] | 1336 |
| 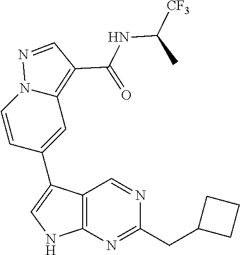 [SMILES: C[C@@H](NC(=O)c1cnn2ccc(-c3c[nH]c4nc(CC5CCC5)ncc34)cc12)C(F)(F)F] | 1337 |
| 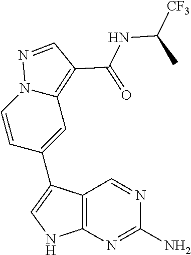 [SMILES: C[C@@H](NC(=O)c1cnn2ccc(-c3c[nH]c4nc(N)ncc34)cc12)C(F)(F)F] | 1338 |
| 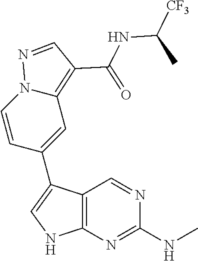 [SMILES: CNc1ncc2c(-c3ccn4ncc(C(=O)N[C@H](C)C(F)(F)F)c4c3)c[nH]c2n1] | 1339 |
| 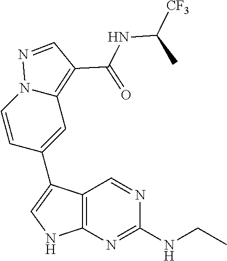 [SMILES: CCNc1ncc2c(-c3ccn4ncc(C(=O)N[C@H](C)C(F)(F)F)c4c3)c[nH]c2n1] | 1340 |
| 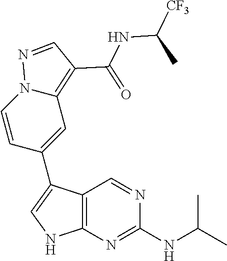 [SMILES: CC(C)Nc1ncc2c(-c3ccn4ncc(C(=O)N[C@H](C)C(F)(F)F)c4c3)c[nH]c2n1] | 1341 |
| 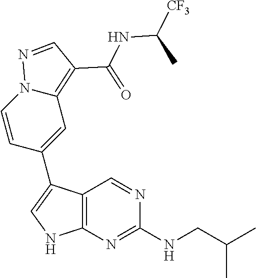 [SMILES: CC(C)CNc1ncc2c(-c3ccn4ncc(C(=O)N[C@H](C)C(F)(F)F)c4c3)c[nH]c2n1] | 1342 |
| 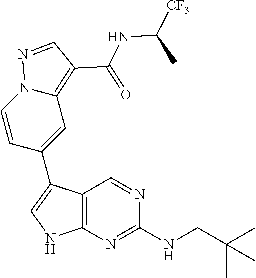 [SMILES: C[C@@H](NC(=O)c1cnn2ccc(-c3c[nH]c4nc(NCC(C)(C)C)ncc34)cc12)C(F)(F)F] | 1343 |
| 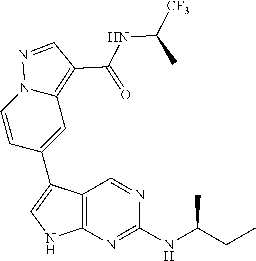 [SMILES: CC[C@H](C)Nc1ncc2c(-c3ccn4ncc(C(=O)N[C@H](C)C(F)(F)F)c4c3)c[nH]c2n1] | 1344 |
| 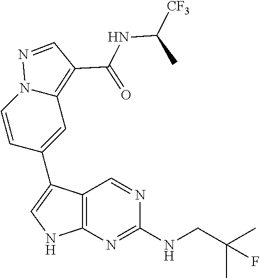 [SMILES: C[C@@H](NC(=O)c1cnn2ccc(-c3c[nH]c4nc(NCC(C)(C)F)ncc34)cc12)C(F)(F)F] | 1345 |
| 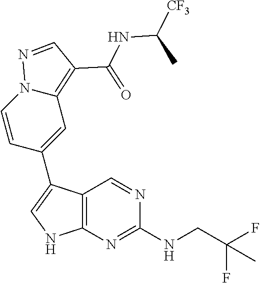 [SMILES: C[C@@H](NC(=O)c1cnn2ccc(-c3c[nH]c4nc(NCC(C)(F)F)ncc34)cc12)C(F)(F)F] | 1346 |
| 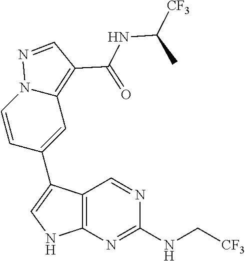 [SMILES: C[C@@H](NC(=O)c1cnn2ccc(-c3c[nH]c4nc(NCC(F)(F)F)ncc34)cc12)C(F)(F)F] | 1347 |
| 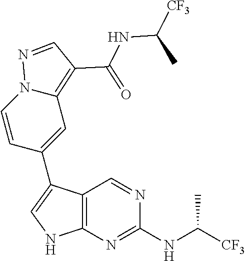 [SMILES: C[C@@H](NC(=O)c1cnn2ccc(-c3c[nH]c4nc(N[C@H](C)C(F)(F)F)ncc34)cc12)C(F)(F)F] | 1348 |
| 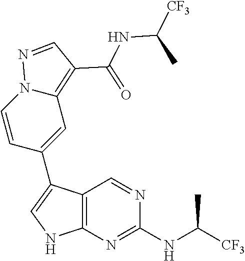 [SMILES: C[C@H](Nc1ncc2c(-c3ccn4ncc(C(=O)N[C@H](C)C(F)(F)F)c4c3)c[nH]c2n1)C(F)(F)F] | 1349 |
| 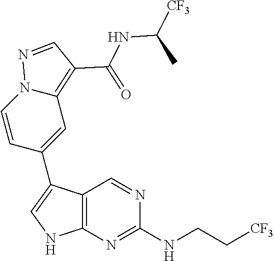 [SMILES: C[C@@H](NC(=O)c1cnn2ccc(-c3c[nH]c4nc(NCCC(F)(F)F)ncc34)cc12)C(F)(F)F] | 1350 |
| 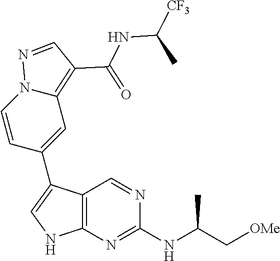 [SMILES: COC[C@H](C)Nc1ncc2c(-c3ccn4ncc(C(=O)N[C@H](C)C(F)(F)F)c4c3)c[nH]c2n1] | 1351 |
| 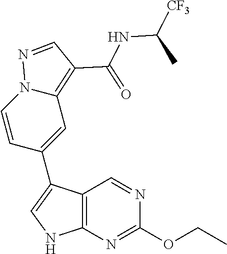 [SMILES: CCOc1ncc2c(-c3ccn4ncc(C(=O)N[C@H](C)C(F)(F)F)c4c3)c[nH]c2n1] | 1352 |
| 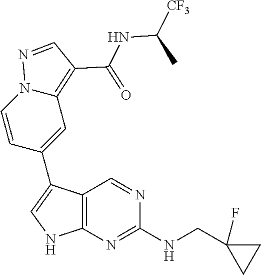 [SMILES: C[C@@H](NC(=O)c1cnn2ccc(-c3c[nH]c4nc(NCC5(F)CC5)ncc34)cc12)C(F)(F)F] | 1353 |
| 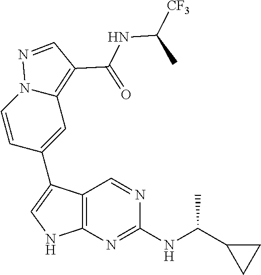 [SMILES: C[C@@H](Nc1ncc2c(-c3ccn4ncc(C(=O)N[C@H](C)C(F)(F)F)c4c3)c[nH]c2n1)C1CC1] | 1354 |
| 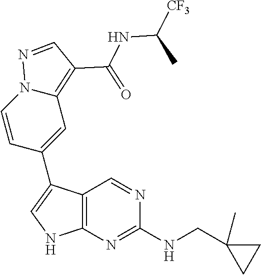 [SMILES: C[C@@H](NC(=O)c1cnn2ccc(-c3c[nH]c4nc(NCC5(C)CC5)ncc34)cc12)C(F)(F)F] | 1355 |
| 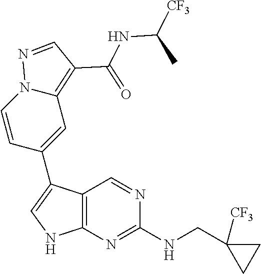 [SMILES: C[C@@H](NC(=O)c1cnn2ccc(-c3c[nH]c4nc(NCC5(C(F)(F)F)CC5)ncc34)cc12)C(F)(F)F] | 1356 |
| 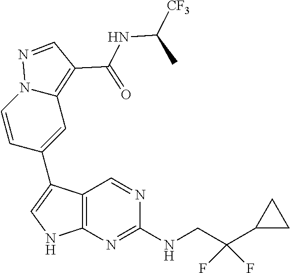 [SMILES: C[C@@H](NC(=O)c1cnn2ccc(-c3c[nH]c4nc(NCC(F)(F)C5CC5)ncc34)cc12)C(F)(F)F] | 1357 |
| 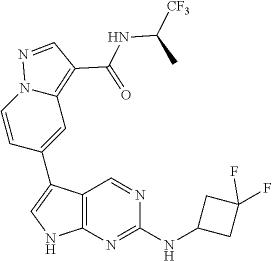 [SMILES: C[C@@H](NC(=O)c1cnn2ccc(-c3c[nH]c4nc(NC5CC(F)(F)C5)ncc34)cc12)C(F)(F)F] | 1358 |
| 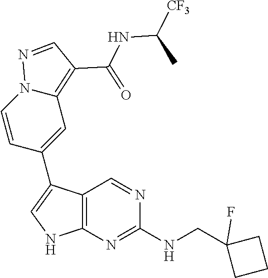 [SMILES: C[C@@H](NC(=O)c1cnn2ccc(-c3c[nH]c4nc(NCC5(F)CCC5)ncc34)cc12)C(F)(F)F] | 1359 |
| 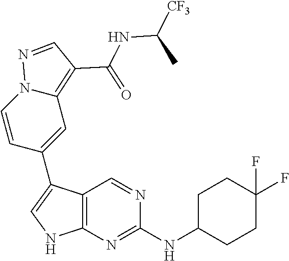 [SMILES: C[C@@H](NC(=O)c1cnn2ccc(-c3c[nH]c4nc(NC5CCC(F)(F)CC5)ncc34)cc12)C(F)(F)F] | 1360 |
| 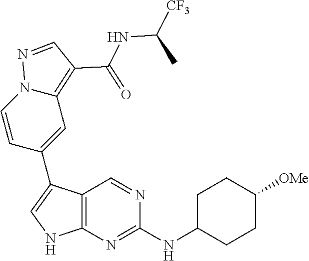 | 1361 |
| 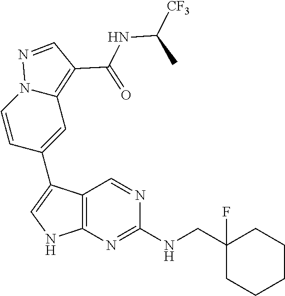 [SMILES: C[C@@H](NC(=O)c1cnn2ccc(-c3c[nH]c4nc(NCC5(F)CCCCC5)ncc34)cc12)C(F)(F)F] | 1362 |
| 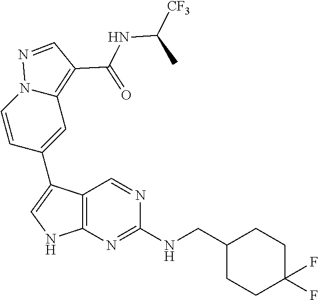 [SMILES: C[C@@H](NC(=O)c1cnn2ccc(-c3c[nH]c4nc(NCC5CCC(F)(F)CC5)ncc34)cc12)C(F)(F)F] | 1363 |
| 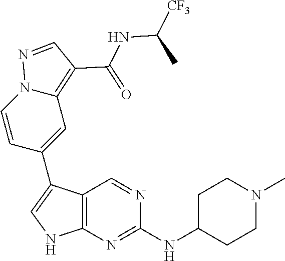 [SMILES: C[C@@H](NC(=O)c1cnn2ccc(-c3c[nH]c4nc(NC5CCN(C)CC5)ncc34)cc12)C(F)(F)F] | 1364 |
| 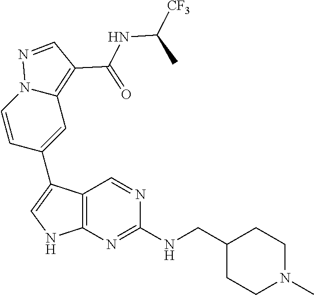 [SMILES: C[C@@H](NC(=O)c1cnn2ccc(-c3c[nH]c4nc(NCC5CCN(C)CC5)ncc34)cc12)C(F)(F)F] | 1365 |
| 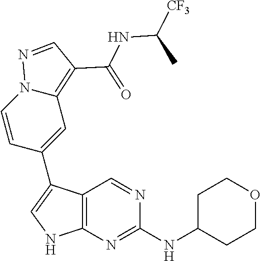 [SMILES: C[C@@H](NC(=O)c1cnn2ccc(-c3c[nH]c4nc(NC5CCOCC5)ncc34)cc12)C(F)(F)F] | 1366 |
| 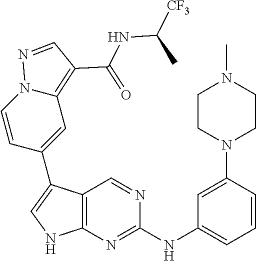 [SMILES: C[C@@H](NC(=O)c1cnn2ccc(-c3c[nH]c4nc(Nc5cccc(N6CCN(C)CC6)c5)ncc34)cc12)C(F)(F)F] | 1367 |
| 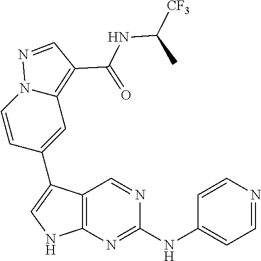 [SMILES: C[C@@H](NC(=O)c1cnn2ccc(-c3c[nH]c4nc(Nc5ccncc5)ncc34)cc12)C(F)(F)F] | 1368 |
| 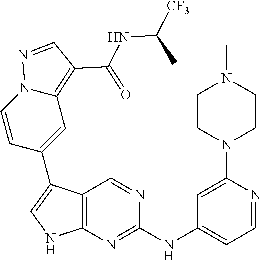 [SMILES: C[C@@H](NC(=O)c1cnn2ccc(-c3c[nH]c4nc(Nc5ccnc(N6CCN(C)CC6)c5)ncc34)cc12)C(F)(F)F] | 1369 |
| 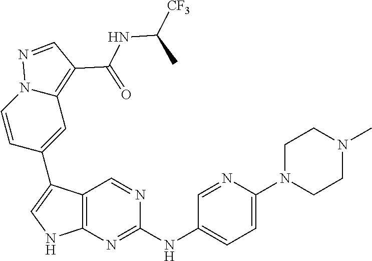 [SMILES: C[C@@H](NC(=O)c1cnn2ccc(-c3c[nH]c4nc(Nc5ccc(N6CCN(C)CC6)nc5)ncc34)cc12)C(F)(F)F] | 1370 |
| 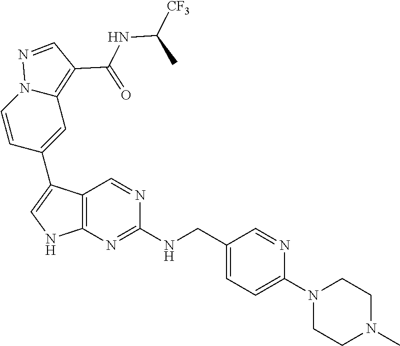 [SMILES: C[C@@H](NC(=O)c1cnn2ccc(-c3c[nH]c4nc(NCc5ccc(N6CCN(C)CC6)nc5)ncc34)cc12)C(F)(F)F] | 1371 |
| 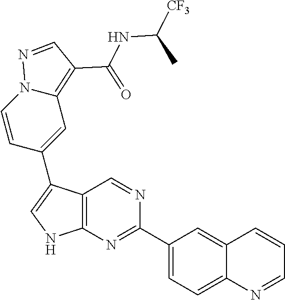 [SMILES: C[C@@H](NC(=O)c1cnn2ccc(-c3c[nH]c4nc(-c5ccc6ncccc6c5)ncc34)cc12)C(F)(F)F] | 1372 |
| 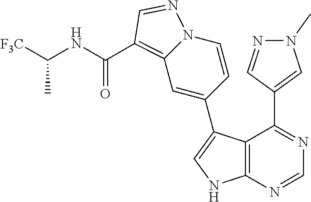 [SMILES: C[C@@H](NC(=O)c1cnn2ccc(-c3c[nH]c4ncnc(-c5cnn(C)c5)c34)cc12)C(F)(F)F] | 1373 |
| 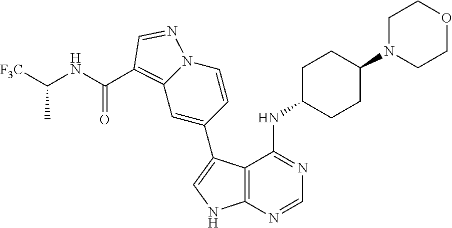 [SMILES: C[C@@H](NC(=O)c1cnn2ccc(-c3c[nH]c4ncnc(N[C@H]5CC[C@H](N6CCOCC6)CC5)c34)cc12)C(F)(F)F] | 1374 |
| 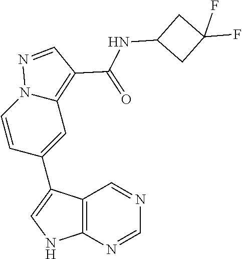 [SMILES: O=C(NC1CC(F)(F)C1)c1cnn2ccc(-c3c[nH]c4ncncc34)cc12] | 1375 |
| 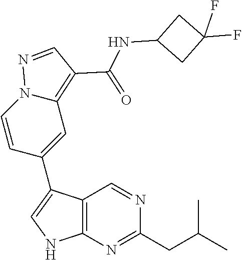 [SMILES: CC(C)Cc1ncc2c(-c3ccn4ncc(C(=O)NC5CC(F)(F)C5)c4c3)c[nH]c2n1] | 1376 |
| 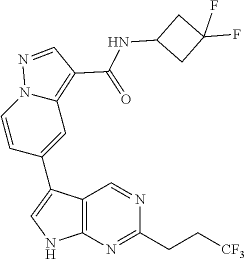 [SMILES: O=C(NC1CC(F)(F)C1)c1cnn2ccc(-c3c[nH]c4nc(CCC(F)(F)F)ncc34)cc12] | 1377 |
| 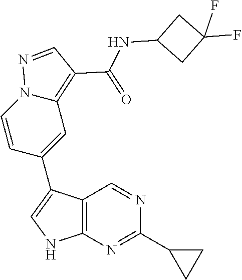 [SMILES: O=C(NC1CC(F)(F)C1)c1cnn2ccc(-c3c[nH]c4nc(C5CC5)ncc34)cc12] | 1378 |
| 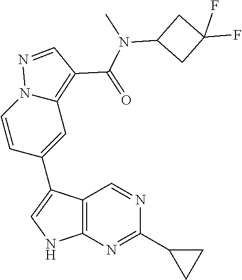 [SMILES: CN(C(=O)c1cnn2ccc(-c3c[nH]c4nc(C5CC5)ncc34)cc12)C1CC(F)(F)C1] | 1379 |
| 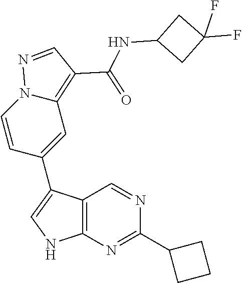 [SMILES: O=C(NC1CC(F)(F)C1)c1cnn2ccc(-c3c[nH]c4nc(C5CCC5)ncc34)cc12] | 1380 |
| 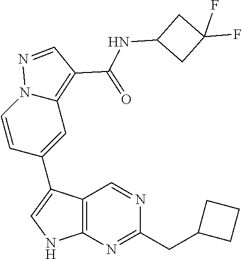 [SMILES: O=C(NC1CC(F)(F)C1)c1cnn2ccc(-c3c[nH]c4nc(CC5CCC5)ncc34)cc12] | 1381 |
| 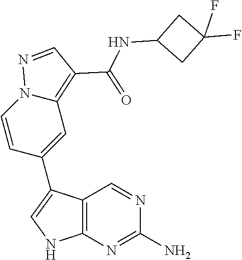 [SMILES: Nc1ncc2c(-c3ccn4ncc(C(=O)NC5CC(F)(F)C5)c4c3)c[nH]c2n1] | 1382 |
| 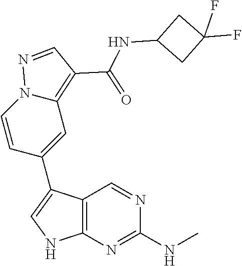 [SMILES: CNc1ncc2c(-c3ccn4ncc(C(=O)NC5CC(F)(F)C5)c4c3)c[nH]c2n1] | 1383 |
| 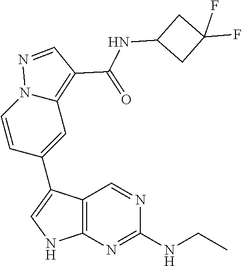 [SMILES: CCNc1ncc2c(-c3ccn4ncc(C(=O)NC5CC(F)(F)C5)c4c3)c[nH]c2n1] | 1384 |
| 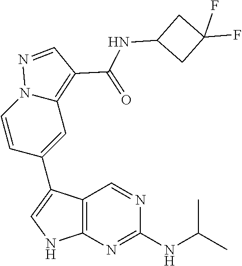 [SMILES: CC(C)Nc1ncc2c(-c3ccn4ncc(C(=O)NC5CC(F)(F)C5)c4c3)c[nH]c2n1] | 1385 |
| 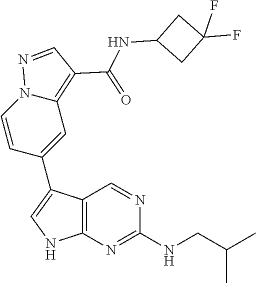 [SMILES: CC(C)CNc1ncc2c(-c3ccn4ncc(C(=O)NC5CC(F)(F)C5)c4c3)c[nH]c2n1] | 1386 |
| 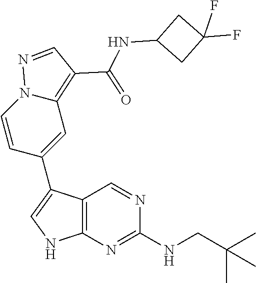 [SMILES: CC(C)(C)CNc1ncc2c(-c3ccn4ncc(C(=O)NC5CC(F)(F)C5)c4c3)c[nH]c2n1] | 1387 |
| 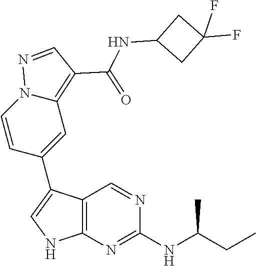 [SMILES: CC[C@H](C)Nc1ncc2c(-c3ccn4ncc(C(=O)NC5CC(F)(F)C5)c4c3)c[nH]c2n1] | 1388 |
| 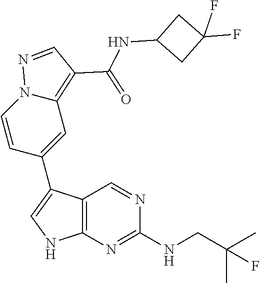 [SMILES: CC(C)(F)CNc1ncc2c(-c3ccn4ncc(C(=O)NC5CC(F)(F)C5)c4c3)c[nH]c2n1] | 1389 |
| 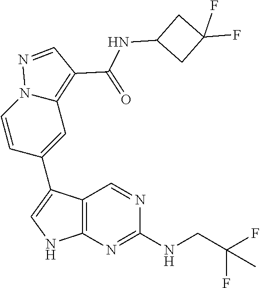 [SMILES: CC(F)(F)CNc1ncc2c(-c3ccn4ncc(C(=O)NC5CC(F)(F)C5)c4c3)c[nH]c2n1] | 1390 |
| 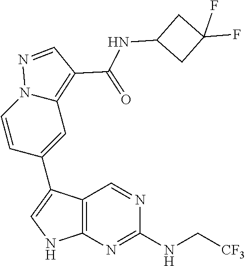 [SMILES: O=C(NC1CC(F)(F)C1)c1cnn2ccc(-c3c[nH]c4nc(NCC(F)(F)F)ncc34)cc12] | 1391 |
| 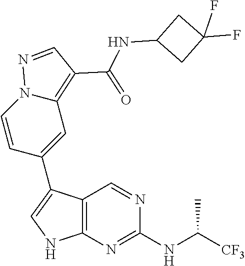 [SMILES: C[C@@H](Nc1ncc2c(-c3ccn4ncc(C(=O)NC5CC(F)(F)C5)c4c3)c[nH]c2n1)C(F)(F)F] | 1392 |
| 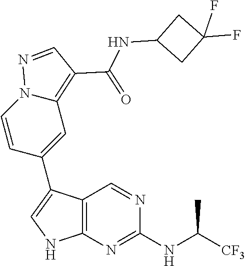 [SMILES: C[C@H](Nc1ncc2c(-c3ccn4ncc(C(=O)NC5CC(F)(F)C5)c4c3)c[nH]c2n1)C(F)(F)F] | 1393 |
| 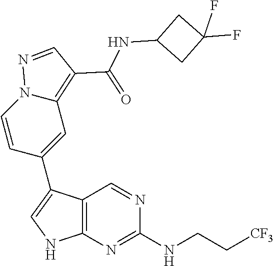 [SMILES: O=C(NC1CC(F)(F)C1)c1cnn2ccc(-c3c[nH]c4nc(NCCC(F)(F)F)ncc34)cc12] | 1394 |
| 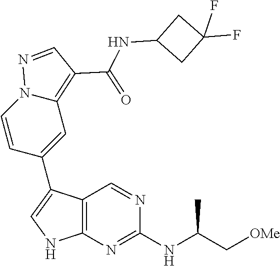 [SMILES: COC[C@H](C)Nc1ncc2c(-c3ccn4ncc(C(=O)NC5CC(F)(F)C5)c4c3)c[nH]c2n1] | 1395 |
| 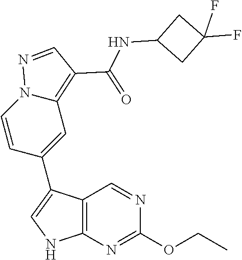 [SMILES: CCOc1ncc2c(-c3ccn4ncc(C(=O)NC5CC(F)(F)C5)c4c3)c[nH]c2n1] | 1396 |
| 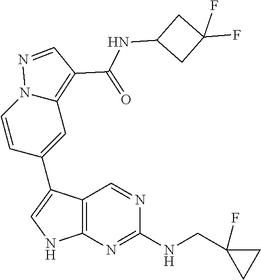 [SMILES: O=C(NC1CC(F)(F)C1)c1cnn2ccc(-c3c[nH]c4nc(NCC5(F)CC5)ncc34)cc12] | 1397 |
| 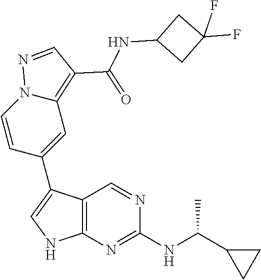 [SMILES: C[C@@H](Nc1ncc2c(-c3ccn4ncc(C(=O)NC5CC(F)(F)C5)c4c3)c[nH]c2n1)C1CC1] | 1398 |
| 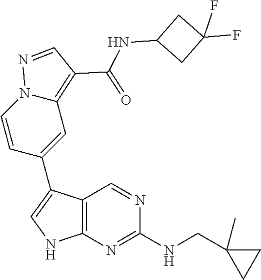 [SMILES: CC1(CNc2ncc3c(-c4ccn5ncc(C(=O)NC6CC(F)(F)C6)c5c4)c[nH]c3n2)CC1] | 1399 |
| 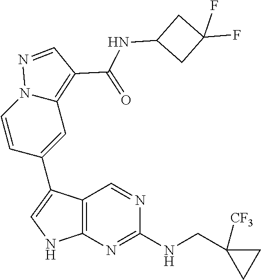 [SMILES: O=C(NC1CC(F)(F)C1)c1cnn2ccc(-c3c[nH]c4nc(NCC5(C(F)(F)F)CC5)ncc34)cc12] | 1400 |
| 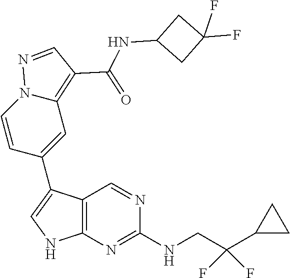 [SMILES: O=C(NC1CC(F)(F)C1)c1cnn2ccc(-c3c[nH]c4nc(NCC(F)(F)C5CC5)ncc34)cc12] | 1401 |
| 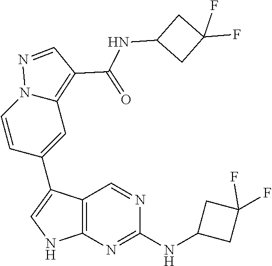 [SMILES: O=C(NC1CC(F)(F)C1)c1cnn2ccc(-c3c[nH]c4nc(NC5CC(F)(F)C5)ncc34)cc12] | 1402 |
| 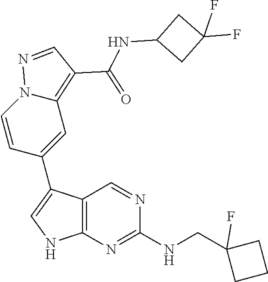 [SMILES: O=C(NC1CC(F)(F)C1)c1cnn2ccc(-c3c[nH]c4nc(NCC5(F)CCC5)ncc34)cc12] | 1403 |
| 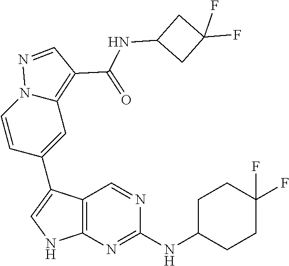 [SMILES: O=C(NC1CC(F)(F)C1)c1cnn2ccc(-c3c[nH]c4nc(NC5CCC(F)(F)CC5)ncc34)cc12] | 1404 |
| 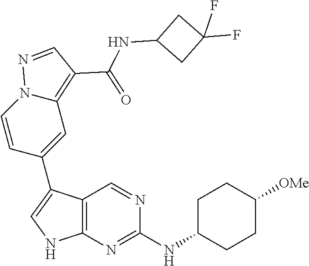 [SMILES: CO[C@H]1CC[C@@H](Nc2ncc3c(-c4ccn5ncc(C(=O)NC6CC(F)(F)C6)c5c4)c[nH]c3n2)CC1] | 1405 |
| 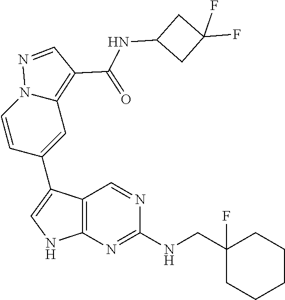 [SMILES: O=C(NC1CC(F)(F)C1)c1cnn2ccc(-c3c[nH]c4nc(NCC5(F)CCCCC5)ncc34)cc12] | 1406 |
| 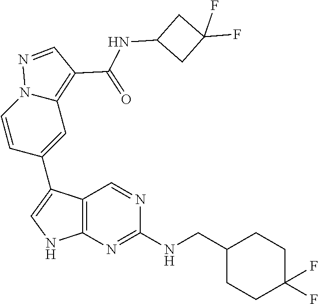 [SMILES: O=C(NC1CC(F)(F)C1)c1cnn2ccc(-c3c[nH]c4nc(NCC5CCC(F)(F)CC5)ncc34)cc12] | 1407 |
| 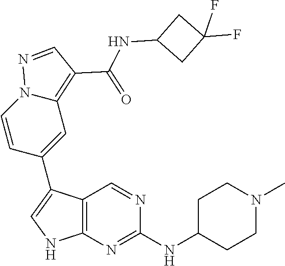 [SMILES: CN1CCC(Nc2ncc3c(-c4ccn5ncc(C(=O)NC6CC(F)(F)C6)c5c4)c[nH]c3n2)CC1] | 1408 |
| 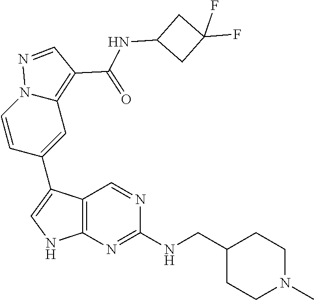 [SMILES: CN1CCC(CNc2ncc3c(-c4ccn5ncc(C(=O)NC6CC(F)(F)C6)c5c4)c[nH]c3n2)CC1] | 1409 |
| 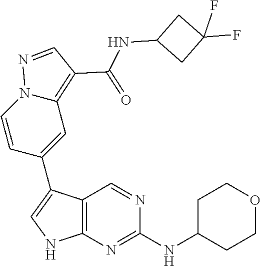 [SMILES: O=C(NC1CC(F)(F)C1)c1cnn2ccc(-c3c[nH]c4nc(NC5CCOCC5)ncc34)cc12] | 1410 |
| 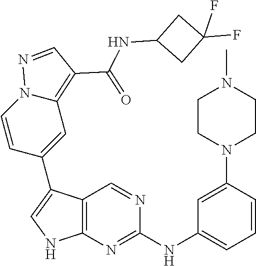 [SMILES: CN1CCN(c2cccc(Nc3ncc4c(-c5ccn6ncc(C(=O)NC7CC(F)(F)C7)c6c5)c[nH]c4n3)c2)CC1] | 1411 |
| 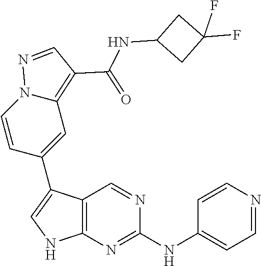 [SMILES: O=C(NC1CC(F)(F)C1)c1cnn2ccc(-c3c[nH]c4nc(Nc5ccncc5)ncc34)cc12] | 1412 |
| 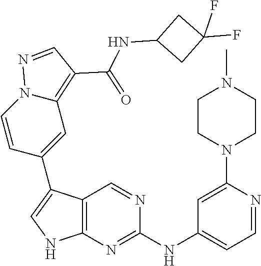 [SMILES: CN1CCN(c2cc(Nc3ncc4c(-c5ccn6ncc(C(=O)NC7CC(F)(F)C7)c6c5)c[nH]c4n3)ccn2)CC1] | 1413 |
| 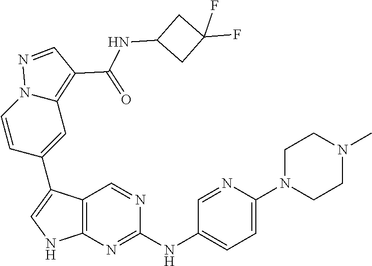 [SMILES: CN1CCN(c2ccc(Nc3ncc4c(-c5ccn6ncc(C(=O)NC7CC(F)(F)C7)c6c5)c[nH]c4n3)cn2)CC1] | 1414 |
| 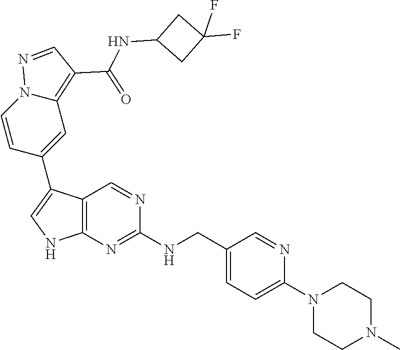 [SMILES: CN1CCN(c2ccc(CNc3ncc4c(-c5ccn6ncc(C(=O)NC7CC(F)(F)C7)c6c5)c[nH]c4n3)cn2)CC1] | 1415 |
| 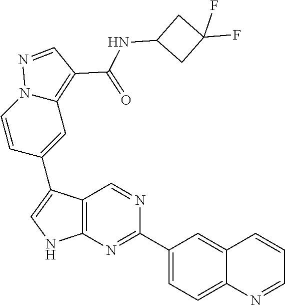 [SMILES: O=C(NC1CC(F)(F)C1)c1cnn2ccc(-c3c[nH]c4nc(-c5ccc6ncccc6c5)ncc34)cc12] | 1416 |
| 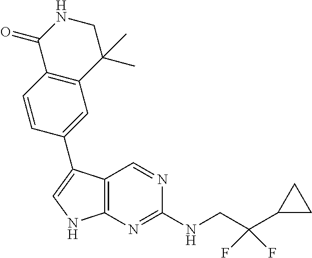 [SMILES: CC1(C)CNC(=O)c2ccc(-c3c[nH]c4nc(NCC(F)(F)C5CC5)ncc34)cc21] | 1417 |
| 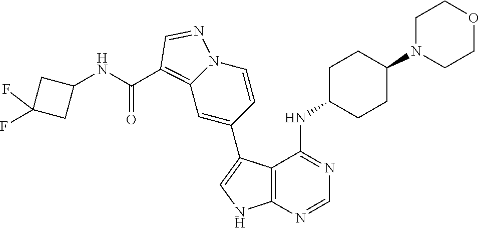 [SMILES: O=C(NC1CC(F)(F)C1)c1cnn2ccc(-c3c[nH]c4ncnc(N[C@H]5CC[C@H](N6CCOCC6)CC5)c34)cc12] | 1418 |
| 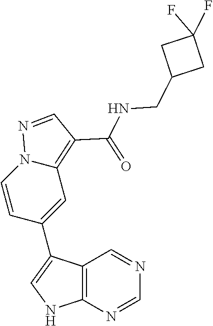 [SMILES: O=C(NCC1CC(F)(F)C1)c1cnn2ccc(-c3c[nH]c4ncncc34)cc12] | 1419 |
| 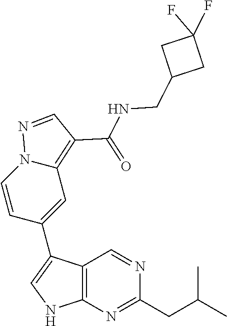 [SMILES: CC(C)Cc1ncc2c(-c3ccn4ncc(C(=O)NCC5CC(F)(F)C5)c4c3)c[nH]c2n1] | 1420 |
| 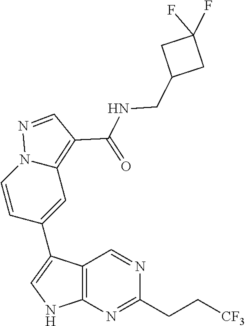 [SMILES: O=C(NCC1CC(F)(F)C1)c1cnn2ccc(-c3c[nH]c4nc(CCC(F)(F)F)ncc34)cc12] | 1421 |
| 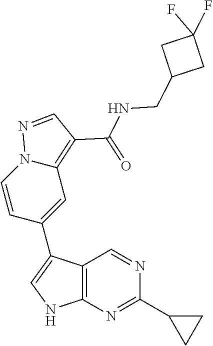 [SMILES: O=C(NCC1CC(F)(F)C1)c1cnn2ccc(-c3c[nH]c4nc(C5CC5)ncc34)cc12] | 1422 |
| 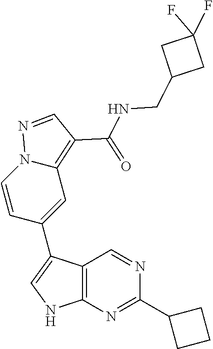 [SMILES: O=C(NCC1CC(F)(F)C1)c1cnn2ccc(-c3c[nH]c4nc(C5CCC5)ncc34)cc12] | 1423 |
| 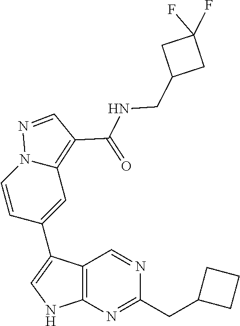 [SMILES: O=C(NCC1CC(F)(F)C1)c1cnn2ccc(-c3c[nH]c4nc(CC5CCC5)ncc34)cc12] | 1424 |
| 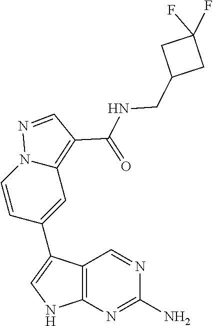 [SMILES: Nc1ncc2c(-c3ccn4ncc(C(=O)NCC5CC(F)(F)C5)c4c3)c[nH]c2n1] | 1425 |
| 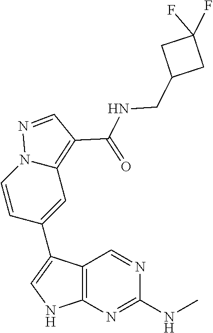 [SMILES: CNc1ncc2c(-c3ccn4ncc(C(=O)NCC5CC(F)(F)C5)c4c3)c[nH]c2n1] | 1426 |
| 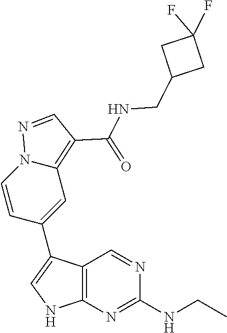 [SMILES: CCNc1ncc2c(-c3ccn4ncc(C(=O)NCC5CC(F)(F)C5)c4c3)c[nH]c2n1] | 1427 |
| 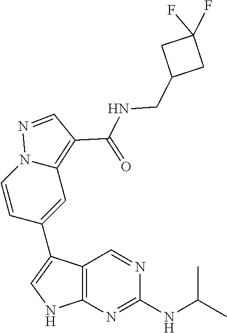 [SMILES: CC(C)Nc1ncc2c(-c3ccn4ncc(C(=O)NCC5CC(F)(F)C5)c4c3)c[nH]c2n1] | 1428 |
| 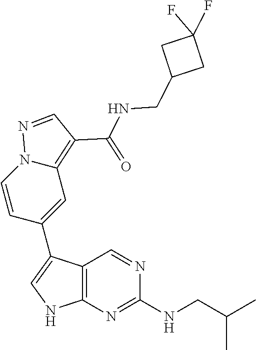 [SMILES: CC(C)CNc1ncc2c(-c3ccn4ncc(C(=O)NCC5CC(F)(F)C5)c4c3)c[nH]c2n1] | 2053 |
| 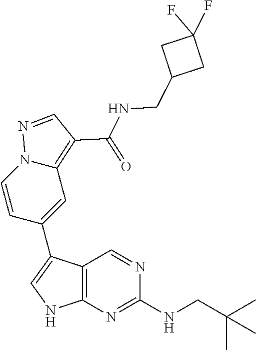 [SMILES: CC(C)(C)CNc1ncc2c(-c3ccn4ncc(C(=O)NCC5CC(F)(F)C5)c4c3)c[nH]c2n1] | 2054 |
| 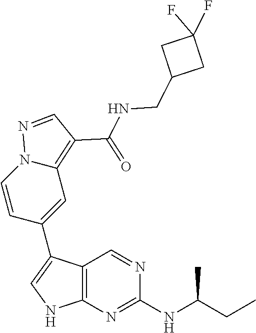 [SMILES: CC[C@H](C)Nc1ncc2c(-c3ccn4ncc(C(=O)NCC5CC(F)(F)C5)c4c3)c[nH]c2n1] | 2055 |
| 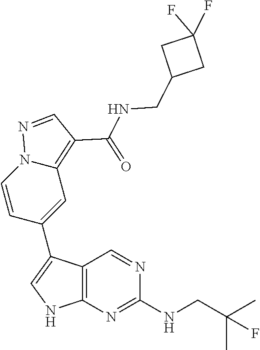 [SMILES: CC(C)(F)CNc1ncc2c(-c3ccn4ncc(C(=O)NCC5CC(F)(F)C5)c4c3)c[nH]c2n1] | 2056 |
| 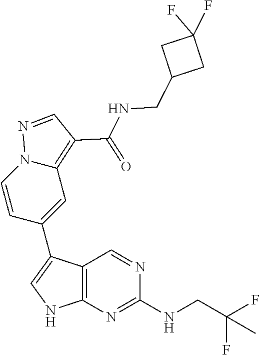 [SMILES: CC(F)(F)CNc1ncc2c(-c3ccn4ncc(C(=O)NCC5CC(F)(F)C5)c4c3)c[nH]c2n1] | 2057 |
| 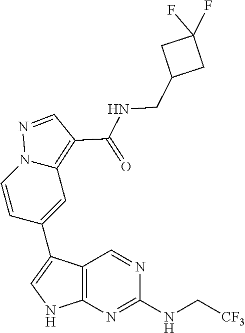 [SMILES: O=C(NCC1CC(F)(F)C1)c1cnn2ccc(-c3c[nH]c4nc(NCC(F)(F)F)ncc34)cc12] | 2058 |
| 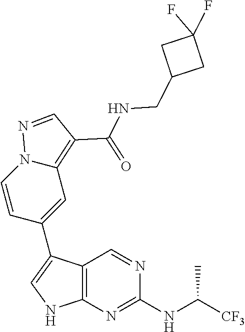 [SMILES: C[C@@H](Nc1ncc2c(-c3ccn4ncc(C(=O)NCC5CC(F)(F)C5)c4c3)c[nH]c2n1)C(F)(F)F] | 2059 |
| 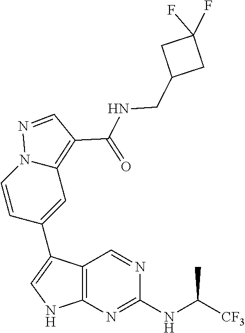 [SMILES: C[C@H](Nc1ncc2c(-c3ccn4ncc(C(=O)NCC5CC(F)(F)C5)c4c3)c[nH]c2n1)C(F)(F)F] | 2060 |
| 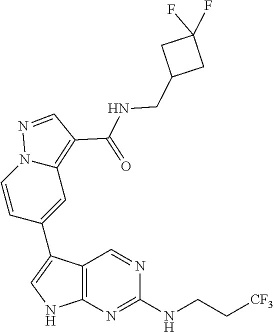 [SMILES: O=C(NCC1CC(F)(F)C1)c1cnn2ccc(-c3c[nH]c4nc(NCCC(F)(F)F)ncc34)cc12] | 2061 |
| 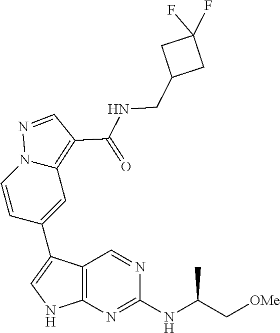 [SMILES: COC[C@H](C)Nc1ncc2c(-c3ccn4ncc(C(=O)NCC5CC(F)(F)C5)c4c3)c[nH]c2n1] | 2062 |
| 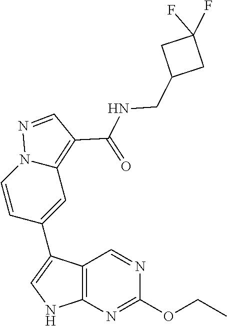 [SMILES: CCOc1ncc2c(-c3ccn4ncc(C(=O)NCC5CC(F)(F)C5)c4c3)c[nH]c2n1] | 2063 |
| 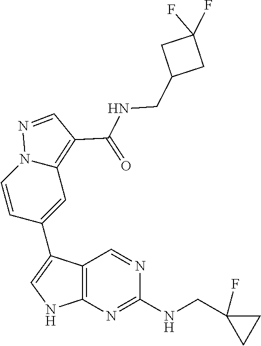 [SMILES: O=C(NCC1CC(F)(F)C1)c1cnn2ccc(-c3c[nH]c4nc(NCC5(F)CC5)ncc34)cc12] | 2064 |
| 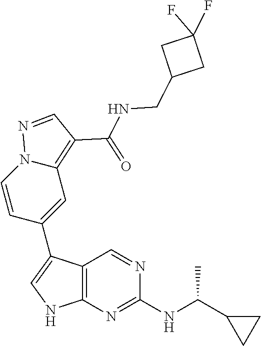 [SMILES: C[C@@H](Nc1ncc2c(-c3ccn4ncc(C(=O)NCC5CC(F)(F)C5)c4c3)c[nH]c2n1)C1CC1] | 2065 |
| 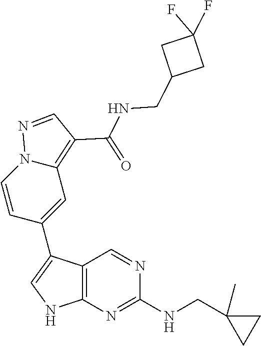 [SMILES: CC1(CNc2ncc3c(-c4ccn5ncc(C(=O)NCC6CC(F)(F)C6)c5c4)c[nH]c3n2)CC1] | 2066 |
| 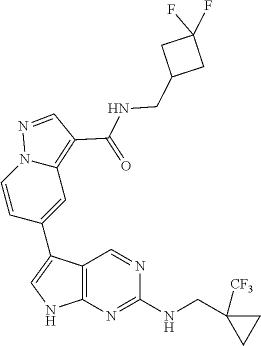 [SMILES: O=C(NCC1CC(F)(F)C1)c1cnn2ccc(-c3c[nH]c4nc(NCC5(C(F)(F)F)CC5)ncc34)cc12] | 2067 |
| 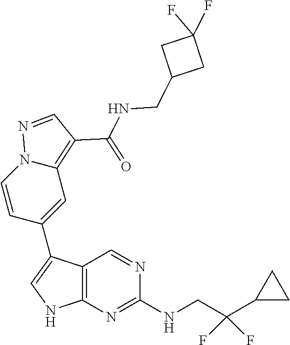 [SMILES: O=C(NCC1CC(F)(F)C1)c1cnn2ccc(-c3c[nH]c4nc(NCC(F)(F)C5CC5)ncc34)cc12] | 1444 |
| 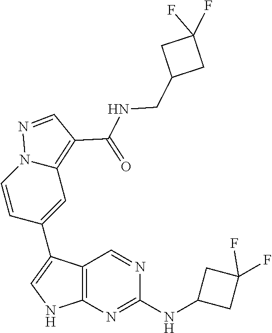 [SMILES: O=C(NCC1CC(F)(F)C1)c1cnn2ccc(-c3c[nH]c4nc(NC5CC(F)(F)C5)ncc34)cc12] | 1445 |
| 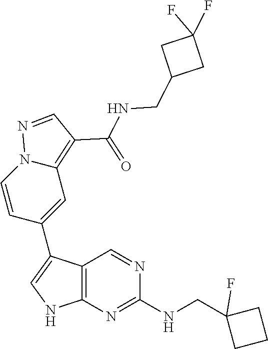 [SMILES: O=C(NCC1CC(F)(F)C1)c1cnn2ccc(-c3c[nH]c4nc(NCC5(F)CCC5)ncc34)cc12] | 1446 |
| 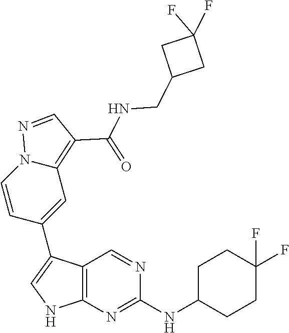 [SMILES: O=C(NCC1CC(F)(F)C1)c1cnn2ccc(-c3c[nH]c4nc(NC5CCC(F)(F)CC5)ncc34)cc12] | 1447 |
| 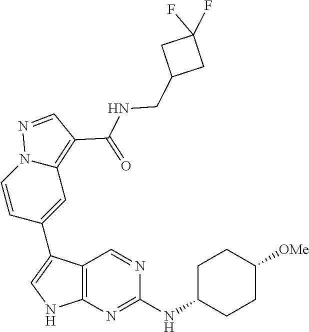 [SMILES: CO[C@H]1CC[C@@H](Nc2ncc3c(-c4ccn5ncc(C(=O)NCC6CC(F)(F)C6)c5c4)c[nH]c3n2)CC1] | 1448 |
| 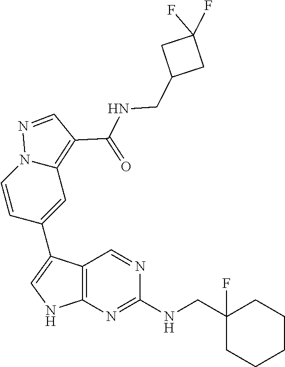 [SMILES: O=C(NCC1CC(F)(F)C1)c1cnn2ccc(-c3c[nH]c4nc(NCC5(F)CCCCC5)ncc34)cc12] | 1449 |
| 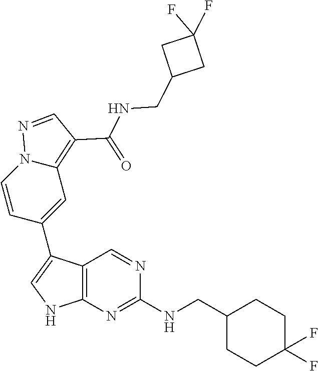 [SMILES: O=C(NCC1CC(F)(F)C1)c1cnn2ccc(-c3c[nH]c4nc(NCC5CCC(F)(F)CC5)ncc34)cc12] | 1450 |
| 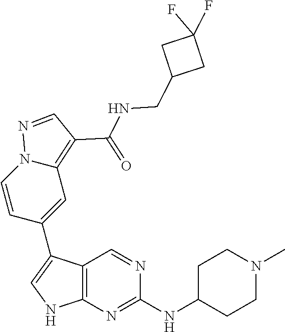 [SMILES: CN1CCC(Nc2ncc3c(-c4ccn5ncc(C(=O)NCC6CC(F)(F)C6)c5c4)c[nH]c3n2)CC1] | 1451 |
| 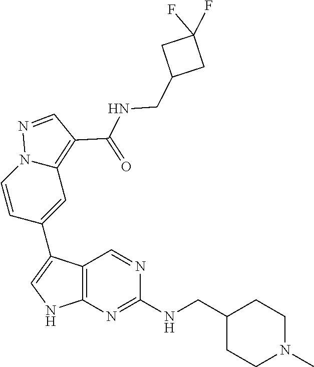 [SMILES: CN1CCC(CNc2ncc3c(-c4ccn5ncc(C(=O)NCC6CC(F)(F)C6)c5c4)c[nH]c3n2)CC1] | 1452 |
| 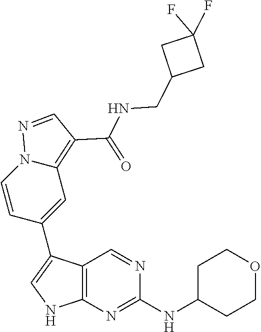 [SMILES: O=C(NCC1CC(F)(F)C1)c1cnn2ccc(-c3c[nH]c4nc(NC5CCOCC5)ncc34)cc12] | 1453 |
| 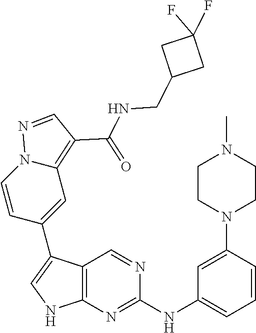 [SMILES: CN1CCN(c2cccc(Nc3ncc4c(-c5ccn6ncc(C(=O)NCC7CC(F)(F)C7)c6c5)c[nH]c4n3)c2)CC1] | 1454 |
| 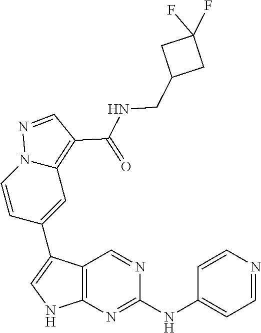 [SMILES: O=C(NCC1CC(F)(F)C1)c1cnn2ccc(-c3c[nH]c4nc(Nc5ccncc5)ncc34)cc12] | 1455 |
| 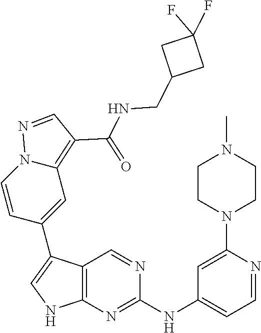 [SMILES: CN1CCN(c2cc(Nc3ncc4c(-c5ccn6ncc(C(=O)NCC7CC(F)(F)C7)c6c5)c[nH]c4n3)ccn2)CC1] | 1456 |
| 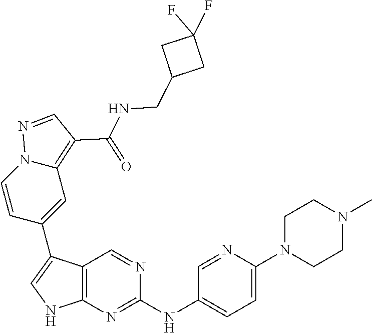 [SMILES: CN1CCN(c2ccc(Nc3ncc4c(-c5ccn6ncc(C(=O)NCC7CC(F)(F)C7)c6c5)c[nH]c4n3)cn2)CC1] | 1457 |
| 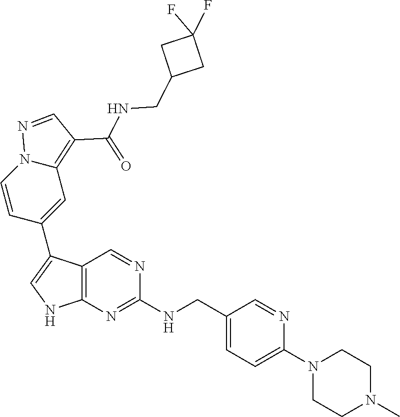 [SMILES: CN1CCN(c2ccc(CNc3ncc4c(-c5ccn6ncc(C(=O)NCC7CC(F)(F)C7)c6c5)c[nH]c4n3)cn2)CC1] | 1458 |
| 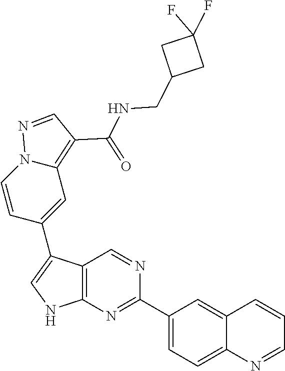 [SMILES: O=C(NCC1CC(F)(F)C1)c1cnn2ccc(-c3c[nH]c4nc(-c5ccc6ncccc6c5)ncc34)cc12] | 1459 |
| 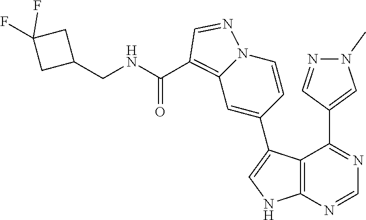 [SMILES: Cn1cc(-c2ncnc3[nH]cc(-c4ccn5ncc(C(=O)NCC6CC(F)(F)C6)c5c4)c23)cn1] | 1460 |
| 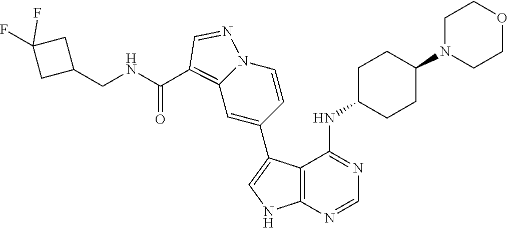 [SMILES: O=C(NCC1CC(F)(F)C1)c1cnn2ccc(-c3c[nH]c4ncnc(N[C@H]5CC[C@H](N6CCOCC6)CC5)c34)cc12] | 1461 |
| 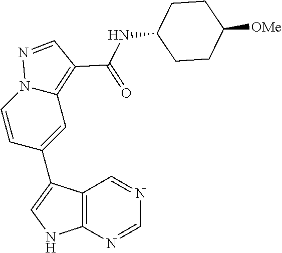 [SMILES: CO[C@H]1CC[C@H](NC(=O)c2cnn3ccc(-c4c[nH]c5ncncc45)cc23)CC1] | 1462 |
| 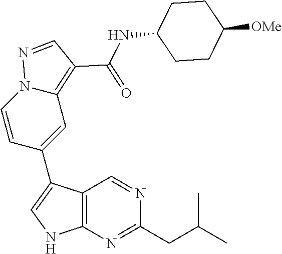 [SMILES: CO[C@H]1CC[C@H](NC(=O)c2cnn3ccc(-c4c[nH]c5nc(CC(C)C)ncc45)cc23)CC1] | 1463 |
| 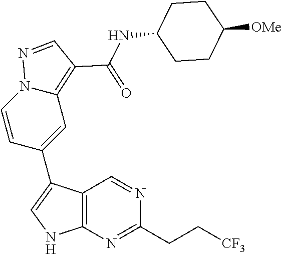 [SMILES: CO[C@H]1CC[C@H](NC(=O)c2cnn3ccc(-c4c[nH]c5nc(CCC(F)(F)F)ncc45)cc23)CC1] | 1464 |
| 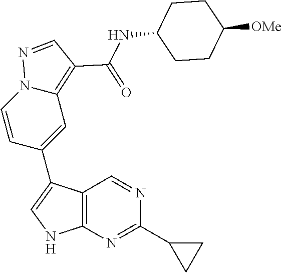 [SMILES: CO[C@H]1CC[C@H](NC(=O)c2cnn3ccc(-c4c[nH]c5nc(C6CC6)ncc45)cc23)CC1] | 1465 |
| 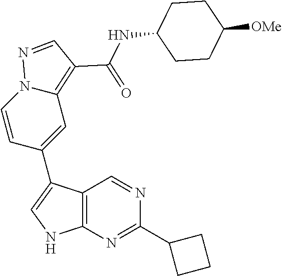 [SMILES: CO[C@H]1CC[C@H](NC(=O)c2cnn3ccc(-c4c[nH]c5nc(C6CCC6)ncc45)cc23)CC1] | 1466 |
| 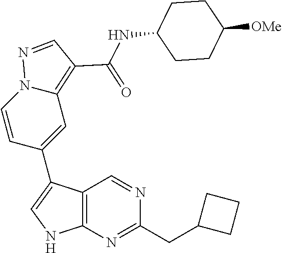 [SMILES: CO[C@H]1CC[C@H](NC(=O)c2cnn3ccc(-c4c[nH]c5nc(CC6CCC6)ncc45)cc23)CC1] | 1467 |
| 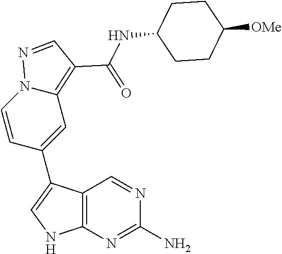 [SMILES: CO[C@H]1CC[C@H](NC(=O)c2cnn3ccc(-c4c[nH]c5nc(N)ncc45)cc23)CC1] | 1468 |
| 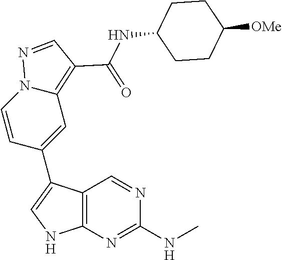 [SMILES: CNc1ncc2c(-c3ccn4ncc(C(=O)N[C@H]5CC[C@H](OC)CC5)c4c3)c[nH]c2n1] | 1469 |
| 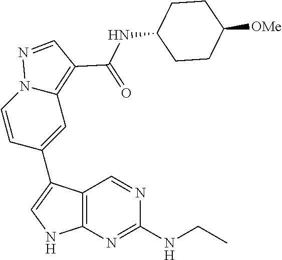 [SMILES: CCNc1ncc2c(-c3ccn4ncc(C(=O)N[C@H]5CC[C@H](OC)CC5)c4c3)c[nH]c2n1] | 1470 |
| 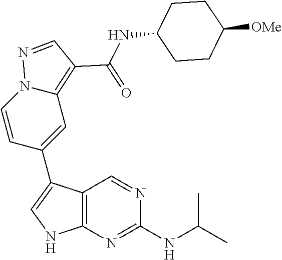 [SMILES: CO[C@H]1CC[C@H](NC(=O)c2cnn3ccc(-c4c[nH]c5nc(NC(C)C)ncc45)cc23)CC1] | 1471 |
| 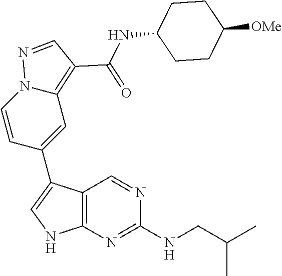 [SMILES: CO[C@H]1CC[C@H](NC(=O)c2cnn3ccc(-c4c[nH]c5nc(NCC(C)C)ncc45)cc23)CC1] | 1472 |
| 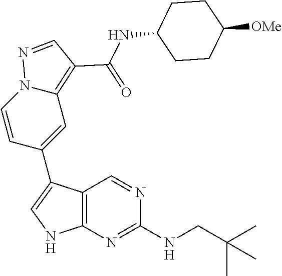 [SMILES: CO[C@H]1CC[C@H](NC(=O)c2cnn3ccc(-c4c[nH]c5nc(NCC(C)(C)C)ncc45)cc23)CC1] | 1473 |
| 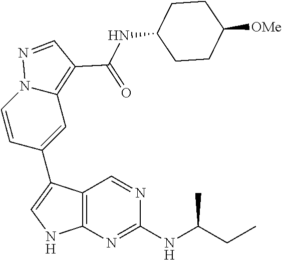 [SMILES: CC[C@H](C)Nc1ncc2c(-c3ccn4ncc(C(=O)N[C@H]5CC[C@H](OC)CC5)c4c3)c[nH]c2n1] | 1474 |
| 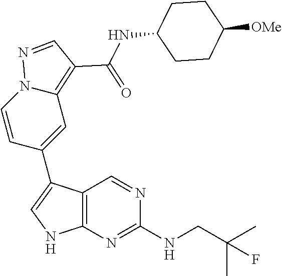 [SMILES: CO[C@H]1CC[C@H](NC(=O)c2cnn3ccc(-c4c[nH]c5nc(NCC(C)(C)F)ncc45)cc23)CC1] | 1475 |
| 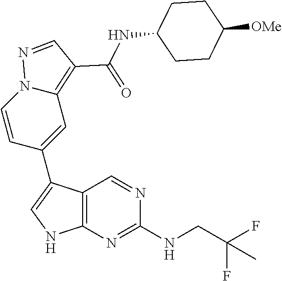 [SMILES: CO[C@H]1CC[C@H](NC(=O)c2cnn3ccc(-c4c[nH]c5nc(NCC(C)(F)F)ncc45)cc23)CC1] | 1476 |
| 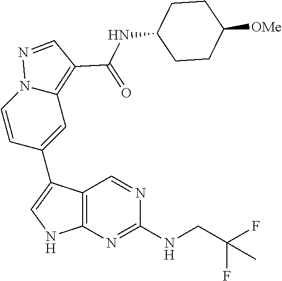 [SMILES: CO[C@H]1CC[C@H](NC(=O)c2cnn3ccc(-c4c[nH]c5nc(NCC(C)(F)F)ncc45)cc23)CC1] | 1477 |
| 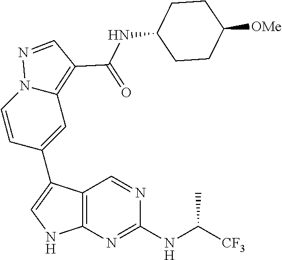 [SMILES: CO[C@H]1CC[C@H](NC(=O)c2cnn3ccc(-c4c[nH]c5nc(N[C@H](C)C(F)(F)F)ncc45)cc23)CC1] | 1478 |
| 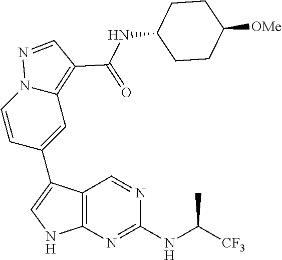 [SMILES: CO[C@H]1CC[C@H](NC(=O)c2cnn3ccc(-c4c[nH]c5nc(N[C@@H](C)C(F)(F)F)ncc45)cc23)CC1] | 1479 |
| 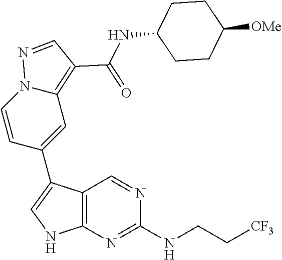 [SMILES: CO[C@H]1CC[C@H](NC(=O)c2cnn3ccc(-c4c[nH]c5nc(NCCC(F)(F)F)ncc45)cc23)CC1] | 1480 |
| 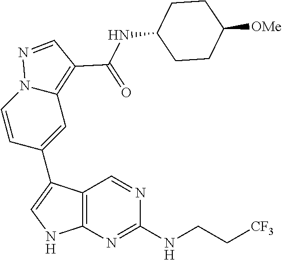 [SMILES: CO[C@H]1CC[C@H](NC(=O)c2cnn3ccc(-c4c[nH]c5nc(NCCC(F)(F)F)ncc45)cc23)CC1] | 1481 |
| 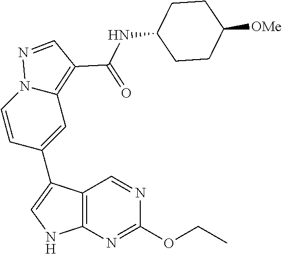 [SMILES: CCOc1ncc2c(-c3ccn4ncc(C(=O)N[C@H]5CC[C@H](OC)CC5)c4c3)c[nH]c2n1] | 1482 |
| 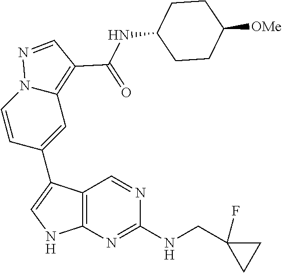 [SMILES: CO[C@H]1CC[C@H](NC(=O)c2cnn3ccc(-c4c[nH]c5nc(NCC6(F)CC6)ncc45)cc23)CC1] | 1483 |
| 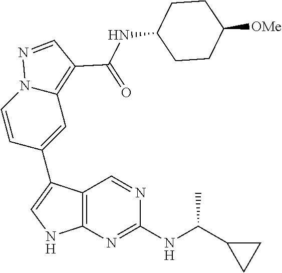 [SMILES: CO[C@H]1CC[C@H](NC(=O)c2cnn3ccc(-c4c[nH]c5nc(N[C@H](C)C6CC6)ncc45)cc23)CC1] | 1484 |
| 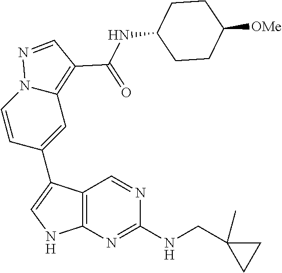 [SMILES: CO[C@H]1CC[C@H](NC(=O)c2cnn3ccc(-c4c[nH]c5nc(NCC6(C)CC6)ncc45)cc23)CC1] | 1485 |
| 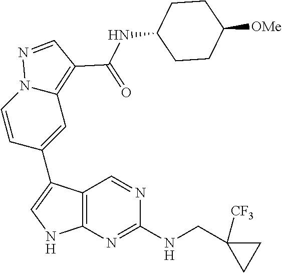 [SMILES: CO[C@H]1CC[C@H](NC(=O)c2cnn3ccc(-c4c[nH]c5nc(NCC6(C(F)(F)F)CC6)ncc45)cc23)CC1] | 1486 |
| 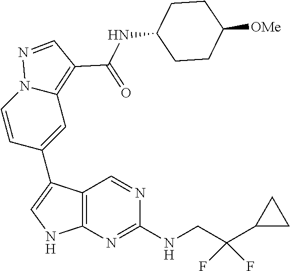 [SMILES: CO[C@H]1CC[C@H](NC(=O)c2cnn3ccc(-c4c[nH]c5nc(NCC(F)(F)C6CC6)ncc45)cc23)CC1] | 1487 |
| 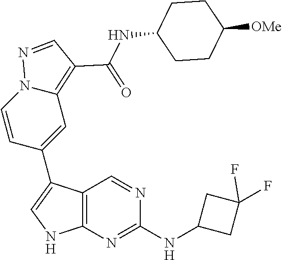 [SMILES: CO[C@H]1CC[C@H](NC(=O)c2cnn3ccc(-c4c[nH]c5nc(NC6CC(F)(F)C6)ncc45)cc23)CC1] | 1488 |
| 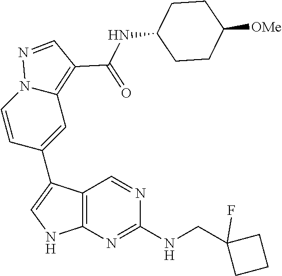 [SMILES: CO[C@H]1CC[C@H](NC(=O)c2cnn3ccc(-c4c[nH]c5nc(NCC6(F)CCC6)ncc45)cc23)CC1] | 1489 |
| 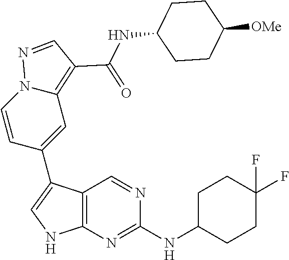 [SMILES: CO[C@H]1CC[C@H](NC(=O)c2cnn3ccc(-c4c[nH]c5nc(NC6CCC(F)(F)CC6)ncc45)cc23)CC1] | 1490 |
| 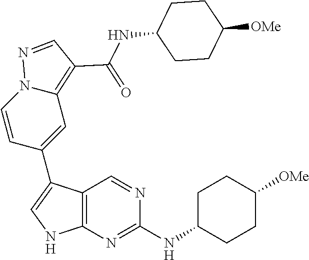 [SMILES: CO[C@H]1CC[C@H](NC(=O)c2cnn3ccc(-c4c[nH]c5nc(N[C@H]6CC[C@@H](OC)CC6)ncc45)cc23)CC1] | 1491 |
| 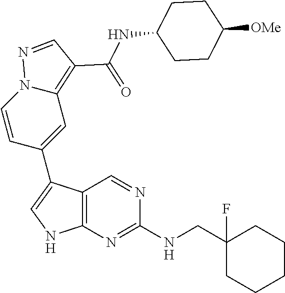 [SMILES: CO[C@H]1CC[C@H](NC(=O)c2cnn3ccc(-c4c[nH]c5nc(NCC6(F)CCCCC6)ncc45)cc23)CC1] | 1492 |
| 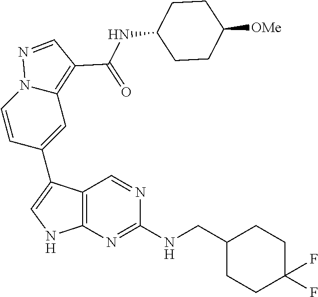 [SMILES: CO[C@H]1CC[C@H](NC(=O)c2cnn3ccc(-c4c[nH]c5nc(NCC6CCC(F)(F)CC6)ncc45)cc23)CC1] | 1493 |
| 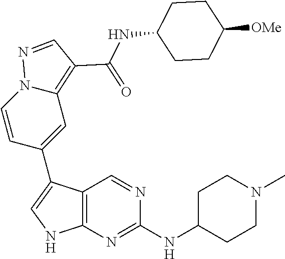 [SMILES: CO[C@H]1CC[C@H](NC(=O)c2cnn3ccc(-c4c[nH]c5nc(NC6CCN(C)CC6)ncc45)cc23)CC1] | 1494 |
| 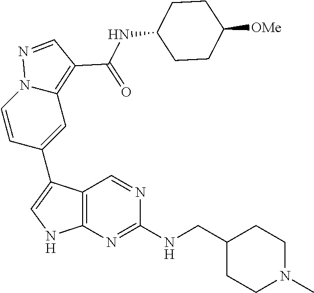 [SMILES: CO[C@H]1CC[C@H](NC(=O)c2cnn3ccc(-c4c[nH]c5nc(NCC6CCN(C)CC6)ncc45)cc23)CC1] | 1495 |
| 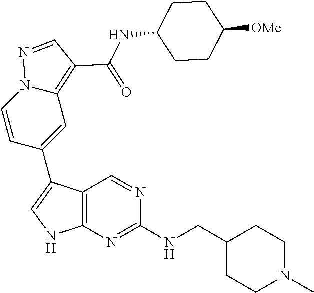 [SMILES: CO[C@H]1CC[C@H](NC(=O)c2cnn3ccc(-c4c[nH]c5nc(NCC6CCN(C)CC6)ncc45)cc23)CC1] | 1496 |
| 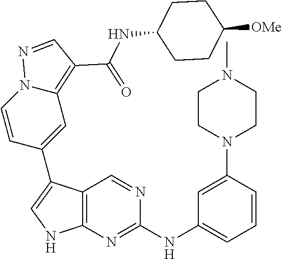 [SMILES: CO[C@H]1CC[C@H](NC(=O)c2cnn3ccc(-c4c[nH]c5nc(Nc6cccc(N7CCN(C)CC7)c6)ncc45)cc23)CC1] | 1497 |
| 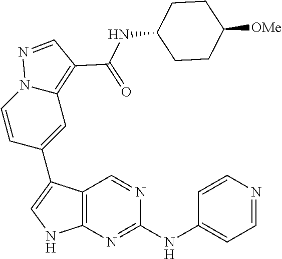 [SMILES: CO[C@H]1CC[C@H](NC(=O)c2cnn3ccc(-c4c[nH]c5nc(Nc6ccncc6)ncc45)cc23)CC1] | 1498 |
| 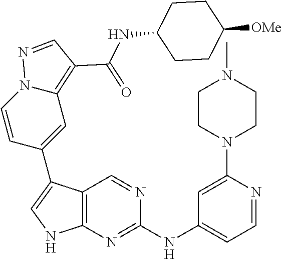 [SMILES: CO[C@H]1CC[C@H](NC(=O)c2cnn3ccc(-c4c[nH]c5nc(Nc6ccnc(N7CCN(C)CC7)c6)ncc45)cc23)CC1] | 1499 |
| 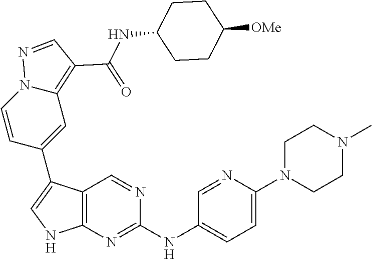 [SMILES: CO[C@H]1CC[C@H](NC(=O)c2cnn3ccc(-c4c[nH]c5nc(Nc6ccc(N7CCN(C)CC7)nc6)ncc45)cc23)CC1] | 1500 |
| 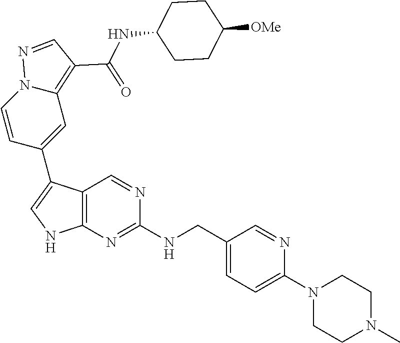 [SMILES: CO[C@H]1CC[C@H](NC(=O)c2cnn3ccc(-c4c[nH]c5nc(NCc6ccc(N7CCN(C)CC7)nc6)ncc45)cc23)CC1] | 1501 |
| 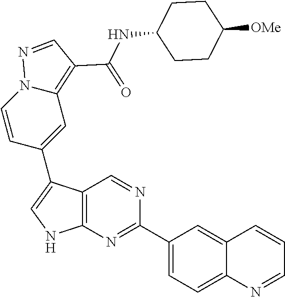 [SMILES: CO[C@H]1CC[C@H](NC(=O)c2cnn3ccc(-c4c[nH]c5nc(-c6ccc7ncccc7c6)ncc45)cc23)CC1] | 1502 |
| 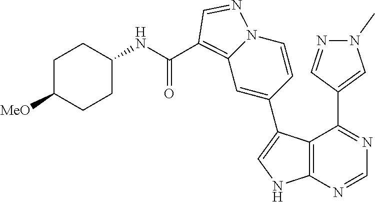 [SMILES: CO[C@H]1CC[C@H](NC(=O)c2cnn3ccc(-c4c[nH]c5ncnc(-c6cnn(C)c6)c45)cc23)CC1] | 1503 |
| 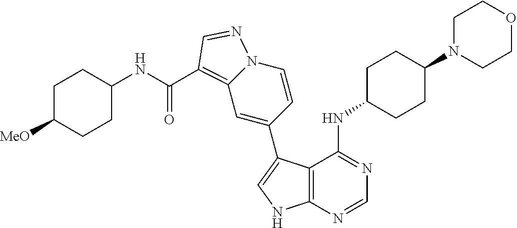 | 1504 |
| 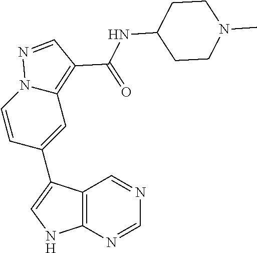 [SMILES: CN1CCC(NC(=O)c2cnn3ccc(-c4c[nH]c5ncncc45)cc23)CC1] | 1505 |
| 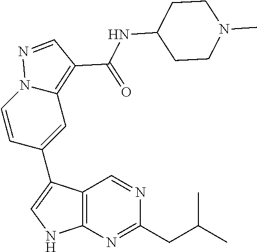 [SMILES: CC(C)Cc1ncc2c(-c3ccn4ncc(C(=O)NC5CCN(C)CC5)c4c3)c[nH]c2n1] | 1506 |
| 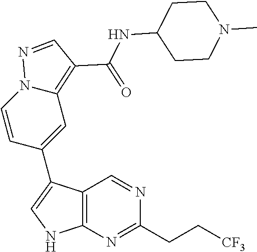 [SMILES: CN1CCC(NC(=O)c2cnn3ccc(-c4c[nH]c5nc(CCC(F)(F)F)ncc45)cc23)CC1] | 1507 |
| 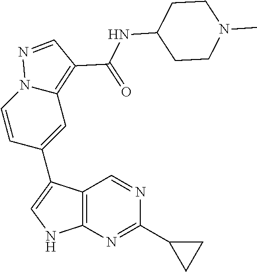 [SMILES: CN1CCC(NC(=O)c2cnn3ccc(-c4c[nH]c5nc(C6CC6)ncc45)cc23)CC1] | 1508 |
| 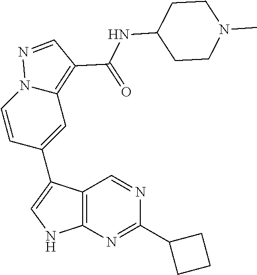 [SMILES: CN1CCC(NC(=O)c2cnn3ccc(-c4c[nH]c5nc(C6CCC6)ncc45)cc23)CC1] | 1509 |
| 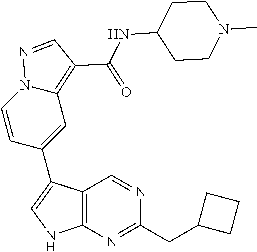 [SMILES: CN1CCC(NC(=O)c2cnn3ccc(-c4c[nH]c5nc(CC6CCC6)ncc45)cc23)CC1] | 1510 |
| 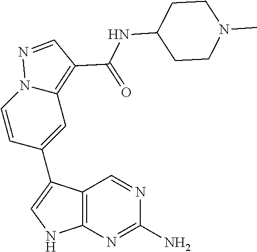 [SMILES: CN1CCC(NC(=O)c2cnn3ccc(-c4c[nH]c5nc(N)ncc45)cc23)CC1] | 1511 |
| 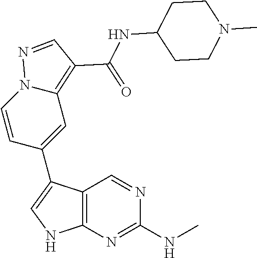 [SMILES: CNc1ncc2c(-c3ccn4ncc(C(=O)NC5CCN(C)CC5)c4c3)c[nH]c2n1] | 1512 |
| 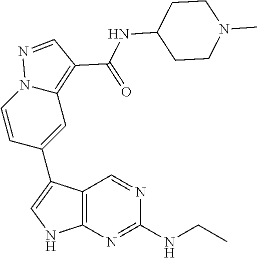 [SMILES: CCNc1ncc2c(-c3ccn4ncc(C(=O)NC5CCN(C)CC5)c4c3)c[nH]c2n1] | 1513 |
| 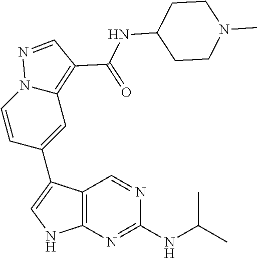 [SMILES: CC(C)Nc1ncc2c(-c3ccn4ncc(C(=O)NC5CCN(C)CC5)c4c3)c[nH]c2n1] | 1514 |
| 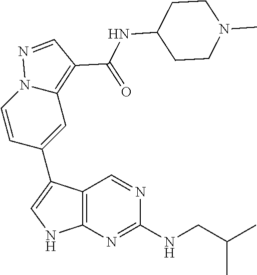 [SMILES: CC(C)CNc1ncc2c(-c3ccn4ncc(C(=O)NC5CCN(C)CC5)c4c3)c[nH]c2n1] | 1515 |
| 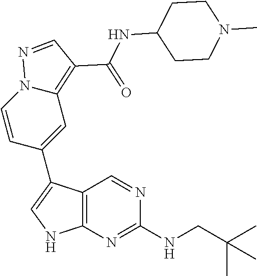 [SMILES: CN1CCC(NC(=O)c2cnn3ccc(-c4c[nH]c5nc(NCC(C)(C)C)ncc45)cc23)CC1] | 1516 |
| 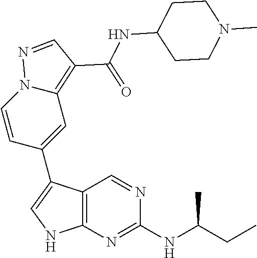 [SMILES: CC[C@H](C)Nc1ncc2c(-c3ccn4ncc(C(=O)NC5CCN(C)CC5)c4c3)c[nH]c2n1] | 1517 |
| 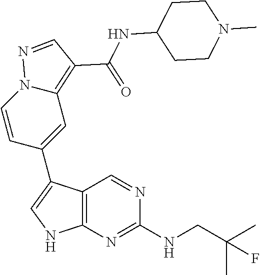 [SMILES: CN1CCC(NC(=O)c2cnn3ccc(-c4c[nH]c5nc(NCC(C)(C)F)ncc45)cc23)CC1] | 1518 |
| 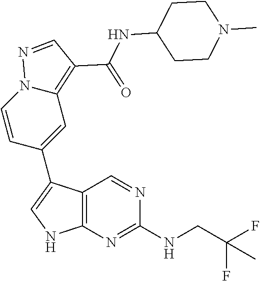 [SMILES: CN1CCC(NC(=O)c2cnn3ccc(-c4c[nH]c5nc(NCC(C)(F)F)ncc45)cc23)CC1] | 1519 |
| 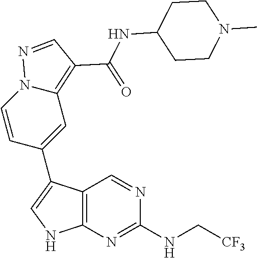 [SMILES: CN1CCC(NC(=O)c2cnn3ccc(-c4c[nH]c5nc(NCC(F)(F)F)ncc45)cc23)CC1] | 1520 |
| 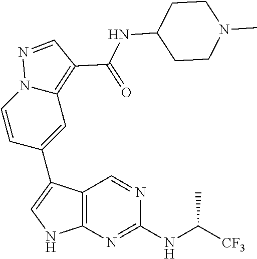 [SMILES: C[C@@H](Nc1ncc2c(-c3ccn4ncc(C(=O)NC5CCN(C)CC5)c4c3)c[nH]c2n1)C(F)(F)F] | 1521 |
| 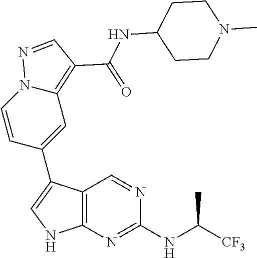 [SMILES: C[C@H](Nc1ncc2c(-c3ccn4ncc(C(=O)NC5CCN(C)CC5)c4c3)c[nH]c2n1)C(F)(F)F] | 1522 |
| 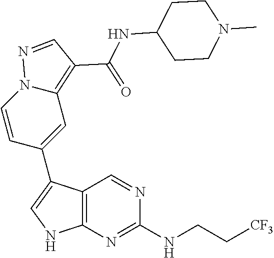 [SMILES: CN1CCC(NC(=O)c2cnn3ccc(-c4c[nH]c5nc(NCCC(F)(F)F)ncc45)cc23)CC1] | 1523 |
| 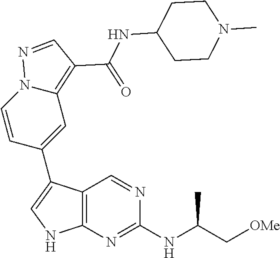 [SMILES: COC[C@H](C)Nc1ncc2c(-c3ccn4ncc(C(=O)NC5CCN(C)CC5)c4c3)c[nH]c2n1] | 1524 |
| 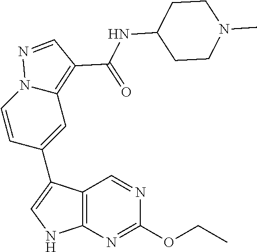 [SMILES: CCOc1ncc2c(-c3ccn4ncc(C(=O)NC5CCN(C)CC5)c4c3)c[nH]c2n1] | 1525 |
| 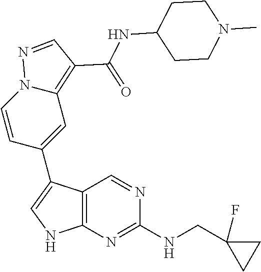 [SMILES: CN1CCC(NC(=O)c2cnn3ccc(-c4c[nH]c5nc(NCC6(F)CC6)ncc45)cc23)CC1] | 1526 |
| 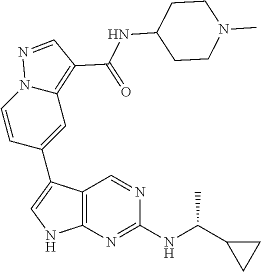 [SMILES: C[C@@H](Nc1ncc2c(-c3ccn4ncc(C(=O)NC5CCN(C)CC5)c4c3)c[nH]c2n1)C1CC1] | 1527 |
| 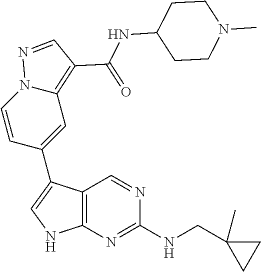 [SMILES: CN1CCC(NC(=O)c2cnn3ccc(-c4c[nH]c5nc(NCC6(C)CC6)ncc45)cc23)CC1] | 1528 |
| 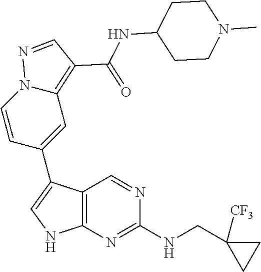 [SMILES: CN1CCC(NC(=O)c2cnn3ccc(-c4c[nH]c5nc(NCC6(C(F)(F)F)CC6)ncc45)cc23)CC1] | 1529 |
| 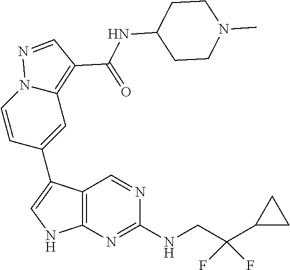 [SMILES: CN1CCC(NC(=O)c2cnn3ccc(-c4c[nH]c5nc(NCC(F)(F)C6CC6)ncc45)cc23)CC1] | 1530 |
| 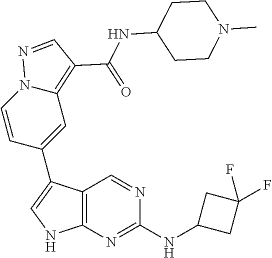 [SMILES: CN1CCC(NC(=O)c2cnn3ccc(-c4c[nH]c5nc(NC6CC(F)(F)C6)ncc45)cc23)CC1] | 1531 |
| 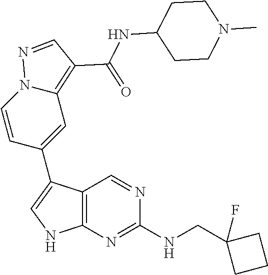 [SMILES: CN1CCC(NC(=O)c2cnn3ccc(-c4c[nH]c5nc(NCC6(F)CCC6)ncc45)cc23)CC1] | 1532 |
| 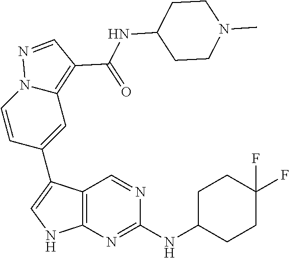 [SMILES: CN1CCC(NC(=O)c2cnn3ccc(-c4c[nH]c5nc(NC6CCC(F)(F)CC6)ncc45)cc23)CC1] | 1533 |
| 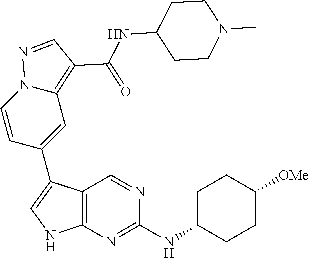 [SMILES: CO[C@H]1CC[C@@H](Nc2ncc3c(-c4ccn5ncc(C(=O)NC6CCN(C)CC6)c5c4)c[nH]c3n2)CC1] | 1534 |
| 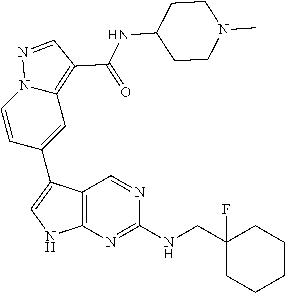 [SMILES: CN1CCC(NC(=O)c2cnn3ccc(-c4c[nH]c5nc(NCC6(F)CCCCC6)ncc45)cc23)CC1] | 1535 |
| 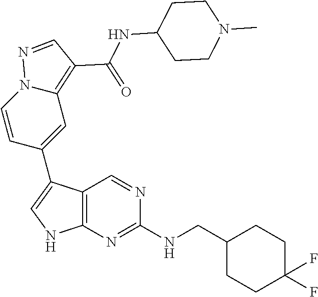 [SMILES: CN1CCC(NC(=O)c2cnn3ccc(-c4c[nH]c5nc(NCC6CCC(F)(F)CC6)ncc45)cc23)CC1] | 1536 |
| 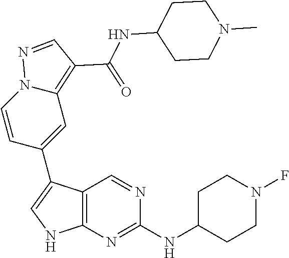 [SMILES: CN1CCC(NC(=O)c2cnn3ccc(-c4c[nH]c5nc(NC6CCN(F)CC6)ncc45)cc23)CC1] | 1537 |
| 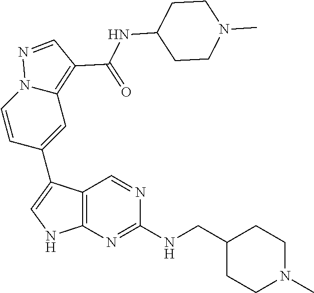 [SMILES: CN1CCC(CNc2ncc3c(-c4ccn5ncc(C(=O)NC6CCN(C)CC6)c5c4)c[nH]c3n2)CC1] | 1538 |
| 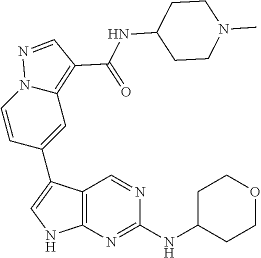 [SMILES: CN1CCC(NC(=O)c2cnn3ccc(-c4c[nH]c5nc(NC6CCOCC6)ncc45)cc23)CC1] | 1539 |
| 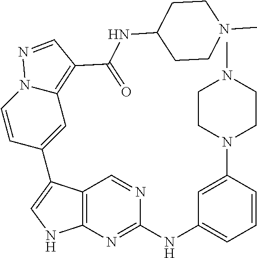 [SMILES: CN1CCC(NC(=O)c2cnn3ccc(-c4c[nH]c5nc(Nc6cccc(N7CCN(C)CC7)c6)ncc45)cc23)CC1] | 1540 |
| 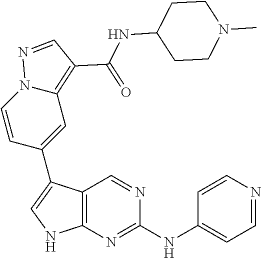 [SMILES: CN1CCC(NC(=O)c2cnn3ccc(-c4c[nH]c5nc(Nc6ccncc6)ncc45)cc23)CC1] | 1541 |
| 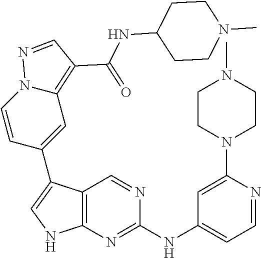 [SMILES: CN1CCC(NC(=O)c2cnn3ccc(-c4c[nH]c5nc(Nc6ccnc(N7CCN(C)CC7)c6)ncc45)cc23)CC1] | 1542 |
| 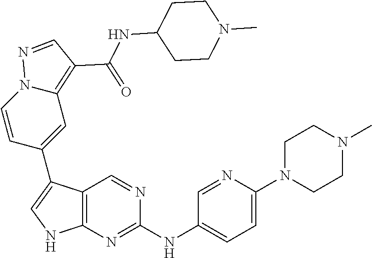 [SMILES: CN1CCC(NC(=O)c2cnn3ccc(-c4c[nH]c5nc(Nc6ccc(N7CCN(C)CC7)nc6)ncc45)cc23)CC1] | 1543 |
| 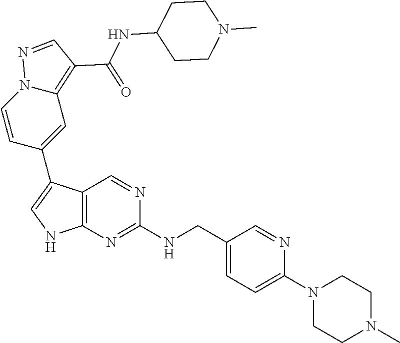 [SMILES: CN1CCC(NC(=O)c2cnn3ccc(-c4c[nH]c5nc(NCc6ccc(N7CCN(C)CC7)nc6)ncc45)cc23)CC1] | 1544 |
| 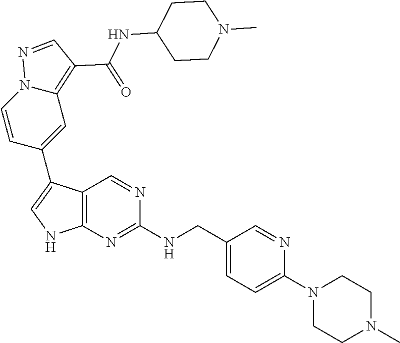 [SMILES: CN1CCC(NC(=O)c2cnn3ccc(-c4c[nH]c5nc(NCc6ccc(N7CCN(C)CC7)nc6)ncc45)cc23)CC1] | 1545 |
| 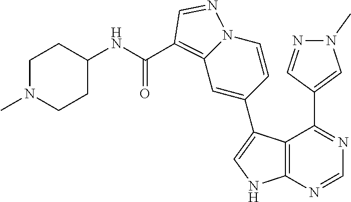 | 1546 |
| 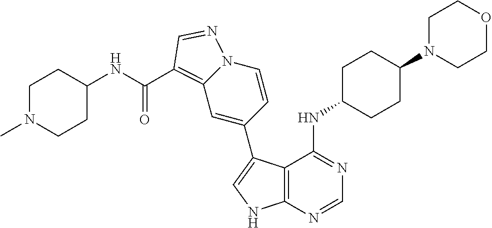 [SMILES: CN1CCC(NC(=O)c2cnn3ccc(-c4c[nH]c5ncnc(N[C@H]6CC[C@H](N7CCOCC7)CC6)c45)cc23)CC1] | 1547 |
| 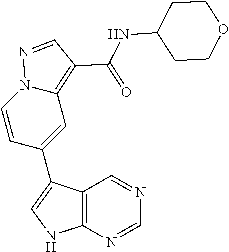 [SMILES: O=C(NC1CCOCC1)c1cnn2ccc(-c3c[nH]c4ncncc34)cc12] | 1548 |
| 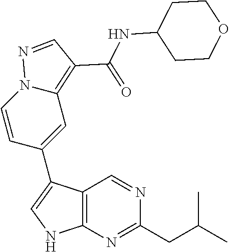 [SMILES: CC(C)Cc1ncc2c(-c3ccn4ncc(C(=O)NC5CCOCC5)c4c3)c[nH]c2n1] | 1549 |
| 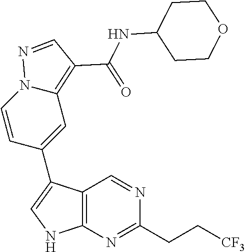 [SMILES: O=C(NC1CCOCC1)c1cnn2ccc(-c3c[nH]c4nc(CCC(F)(F)F)ncc34)cc12] | 1550 |
| 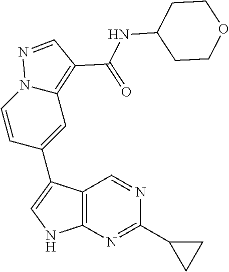 [SMILES: O=C(NC1CCOCC1)c1cnn2ccc(-c3c[nH]c4nc(C5CC5)ncc34)cc12] | 1551 |
| 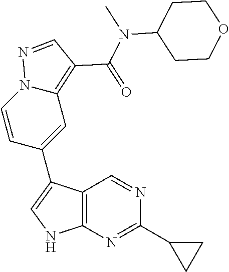 [SMILES: CN(C(=O)c1cnn2ccc(-c3c[nH]c4nc(C5CC5)ncc34)cc12)C1CCOCC1] | 1552 |
| 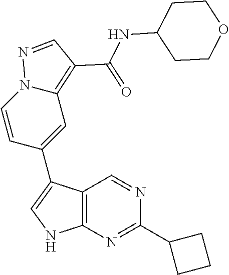 [SMILES: O=C(NC1CCOCC1)c1cnn2ccc(-c3c[nH]c4nc(C5CCC5)ncc34)cc12] | 1553 |
| 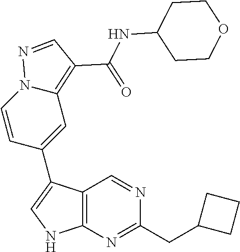 [SMILES: O=C(NC1CCOCC1)c1cnn2ccc(-c3c[nH]c4nc(CC5CCC5)ncc34)cc12] | 1554 |
| 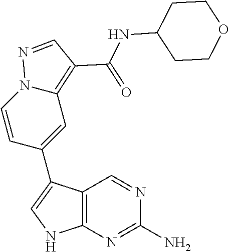 [SMILES: Nc1ncc2c(-c3ccn4ncc(C(=O)NC5CCOCC5)c4c3)c[nH]c2n1] | 1555 |
| 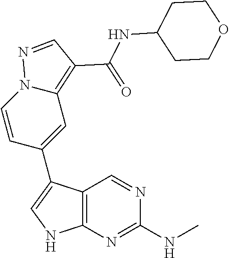 [SMILES: CNc1ncc2c(-c3ccn4ncc(C(=O)NC5CCOCC5)c4c3)c[nH]c2n1] | 1556 |
| 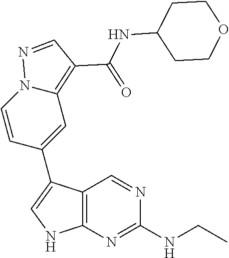 [SMILES: CCNc1ncc2c(-c3ccn4ncc(C(=O)NC5CCOCC5)c4c3)c[nH]c2n1] | 1557 |
| 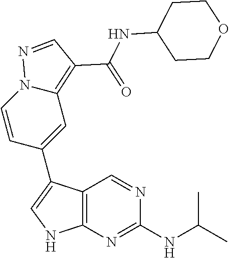 [SMILES: CC(C)Nc1ncc2c(-c3ccn4ncc(C(=O)NC5CCOCC5)c4c3)c[nH]c2n1] | 1558 |
| 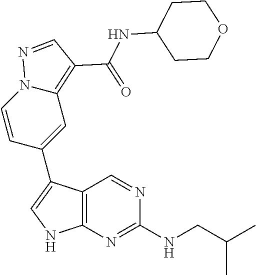 [SMILES: CC(C)CNc1ncc2c(-c3ccn4ncc(C(=O)NC5CCOCC5)c4c3)c[nH]c2n1] | 1559 |
| 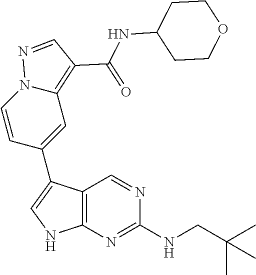 [SMILES: CC(C)(C)CNc1ncc2c(-c3ccn4ncc(C(=O)NC5CCOCC5)c4c3)c[nH]c2n1] | 1560 |
| 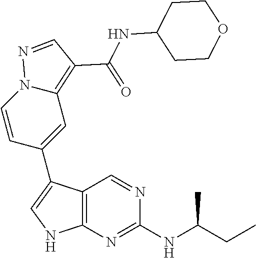 [SMILES: CC[C@H](C)Nc1ncc2c(-c3ccn4ncc(C(=O)NC5CCOCC5)c4c3)c[nH]c2n1] | 1561 |
| 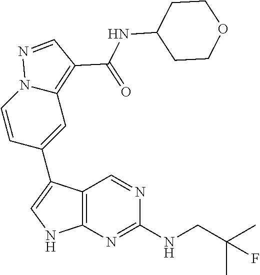 [SMILES: CC(C)(F)CNc1ncc2c(-c3ccn4ncc(C(=O)NC5CCOCC5)c4c3)c[nH]c2n1] | 1562 |
| 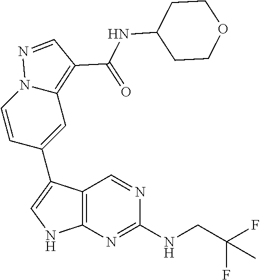 [SMILES: CC(F)(F)CNc1ncc2c(-c3ccn4ncc(C(=O)NC5CCOCC5)c4c3)c[nH]c2n1] | 1563 |
| 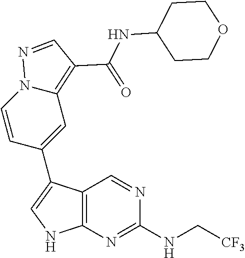 [SMILES: O=C(NC1CCOCC1)c1cnn2ccc(-c3c[nH]c4nc(NCC(F)(F)F)ncc34)cc12] | 1564 |
| 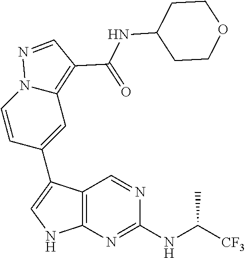 [SMILES: C[C@@H](Nc1ncc2c(-c3ccn4ncc(C(=O)NC5CCOCC5)c4c3)c[nH]c2n1)C(F)(F)F] | 1565 |
| 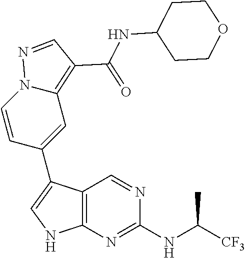 [SMILES: C[C@H](Nc1ncc2c(-c3ccn4ncc(C(=O)NC5CCOCC5)c4c3)c[nH]c2n1)C(F)(F)F] | 1566 |
| 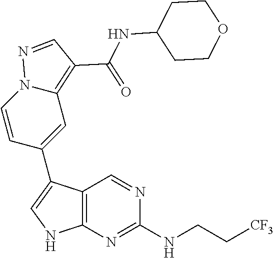 [SMILES: O=C(NC1CCOCC1)c1cnn2ccc(-c3c[nH]c4nc(NCCC(F)(F)F)ncc34)cc12] | 1567 |
| 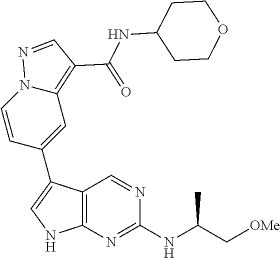 [SMILES: COC[C@H](C)Nc1ncc2c(-c3ccn4ncc(C(=O)NC5CCOCC5)c4c3)c[nH]c2n1] | 1568 |
| 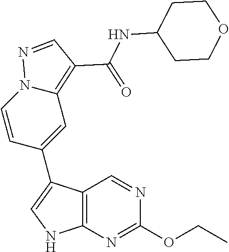 [SMILES: CCOc1ncc2c(-c3ccn4ncc(C(=O)NC5CCOCC5)c4c3)c[nH]c2n1] | 1569 |
| 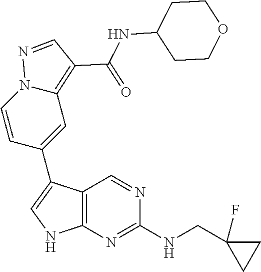 [SMILES: O=C(NC1CCOCC1)c1cnn2ccc(-c3c[nH]c4nc(NCC5(F)CC5)ncc34)cc12] | 1570 |
| 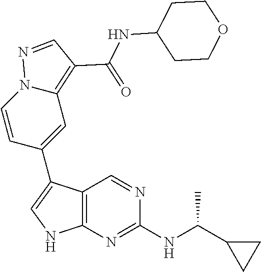 [SMILES: C[C@@H](Nc1ncc2c(-c3ccn4ncc(C(=O)NC5CCOCC5)c4c3)c[nH]c2n1)C1CC1] | 1571 |
| 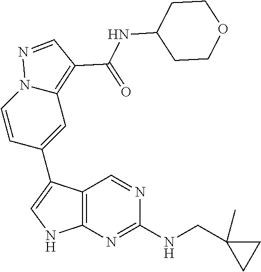 [SMILES: CC1(CNc2ncc3c(-c4ccn5ncc(C(=O)NC6CCOCC6)c5c4)c[nH]c3n2)CC1] | 1572 |
| 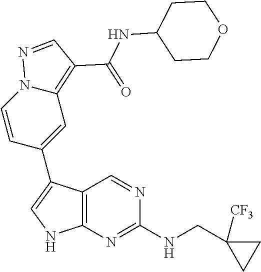 [SMILES: O=C(NC1CCOCC1)c1cnn2ccc(-c3c[nH]c4nc(NCC5(C(F)(F)F)CC5)ncc34)cc12] | 1573 |
| 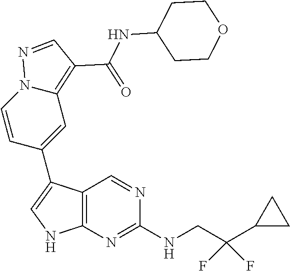 [SMILES: O=C(NC1CCOCC1)c1cnn2ccc(-c3c[nH]c4nc(NCC(F)(F)C5CC5)ncc34)cc12] | 1574 |
| 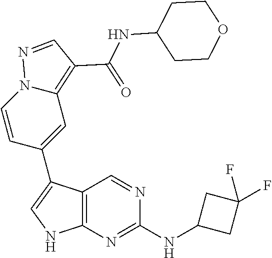 [SMILES: O=C(NC1CCOCC1)c1cnn2ccc(-c3c[nH]c4nc(NC5CC(F)(F)C5)ncc34)cc12] | 1575 |
| 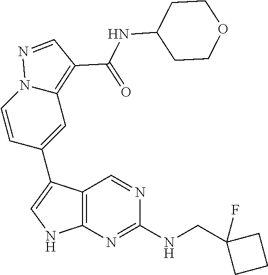 [SMILES: O=C(NC1CCOCC1)c1cnn2ccc(-c3c[nH]c4nc(NCC5(F)CCC5)ncc34)cc12] | 1576 |
| 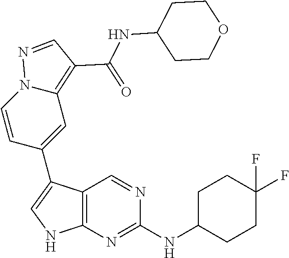 [SMILES: O=C(NC1CCOCC1)c1cnn2ccc(-c3c[nH]c4nc(NC5CCC(F)(F)CC5)ncc34)cc12] | 1577 |
| 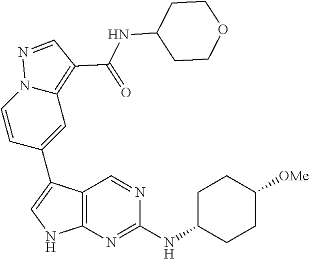 [SMILES: CO[C@H]1CC[C@@H](Nc2ncc3c(-c4ccn5ncc(C(=O)NC6CCOCC6)c5c4)c[nH]c3n2)CC1] | 1578 |
| 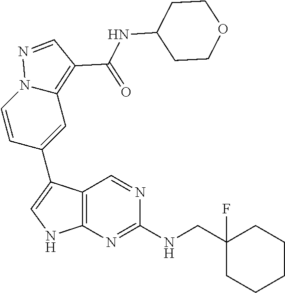 [SMILES: O=C(NC1CCOCC1)c1cnn2ccc(-c3c[nH]c4nc(NCC5(F)CCCCC5)ncc34)cc12] | 1579 |
| 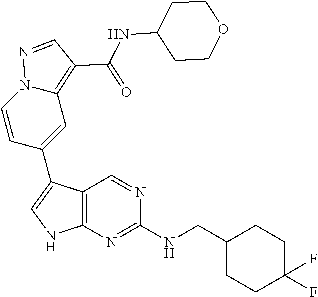 [SMILES: O=C(NC1CCOCC1)c1cnn2ccc(-c3c[nH]c4nc(NCC5CCC(F)(F)CC5)ncc34)cc12] | 1580 |
| 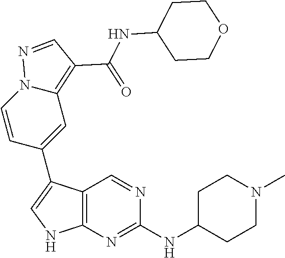 [SMILES: CN1CCC(Nc2ncc3c(-c4ccn5ncc(C(=O)NC6CCOCC6)c5c4)c[nH]c3n2)CC1] | 1581 |
| 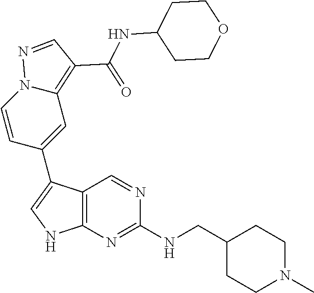 [SMILES: CN1CCC(CNc2ncc3c(-c4ccn5ncc(C(=O)NC6CCOCC6)c5c4)c[nH]c3n2)CC1] | 1582 |
| 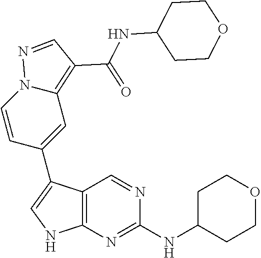 [SMILES: O=C(NC1CCOCC1)c1cnn2ccc(-c3c[nH]c4nc(NC5CCOCC5)ncc34)cc12] | 1583 |
| 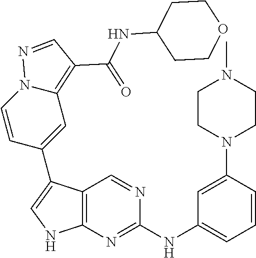 [SMILES: CN1CCN(c2cccc(Nc3ncc4c(-c5ccn6ncc(C(=O)NC7CCOCC7)c6c5)c[nH]c4n3)c2)CC1] | 1584 |
| 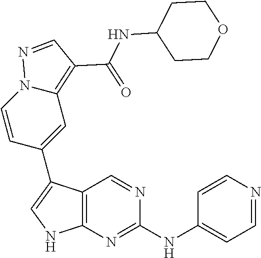 [SMILES: O=C(NC1CCOCC1)c1cnn2ccc(-c3c[nH]c4nc(Nc5ccncc5)ncc34)cc12] | 1585 |
| 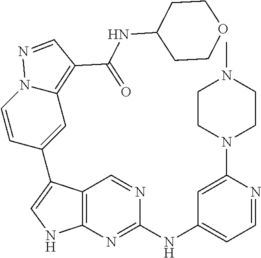 [SMILES: CN1CCN(c2cc(Nc3ncc4c(-c5ccn6ncc(C(=O)NC7CCOCC7)c6c5)c[nH]c4n3)ccn2)CC1] | 1586 |
| 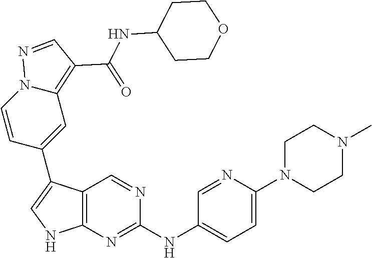 [SMILES: CN1CCN(c2ccc(Nc3ncc4c(-c5ccn6ncc(C(=O)NC7CCOCC7)c6c5)c[nH]c4n3)cn2)CC1] | 1587 |
| 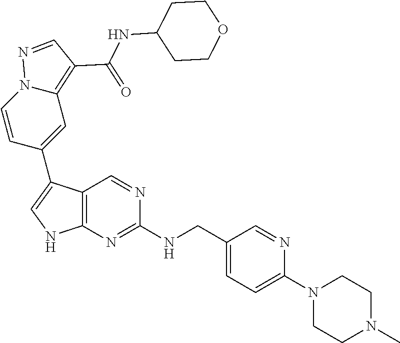 [SMILES: CN1CCN(c2ccc(CNc3ncc4c(-c5ccn6ncc(C(=O)NC7CCOCC7)c6c5)c[nH]c4n3)cn2)CC1] | 1588 |
| 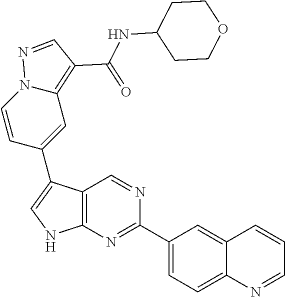 [SMILES: O=C(NC1CCOCC1)c1cnn2ccc(-c3c[nH]c4nc(-c5ccc6ncccc6c5)ncc34)cc12] | 1589 |
| 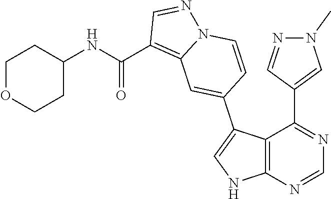 [SMILES: Cn1cc(-c2ncnc3[nH]cc(-c4ccn5ncc(C(=O)NC6CCOCC6)c5c4)c23)cn1] | 1590 |
| 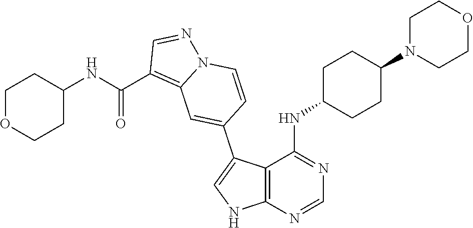 [SMILES: O=C(NC1CCOCC1)c1cnn2ccc(-c3c[nH]c4ncnc(N[C@H]5CC[C@H](N6CCOCC6)CC5)c34)cc12] | 1591 |
| 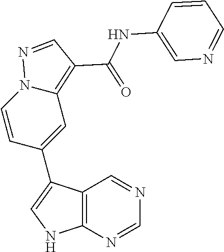 [SMILES: O=C(Nc1cccnc1)c1cnn2ccc(-c3c[nH]c4ncncc34)cc12] | 1592 |
| 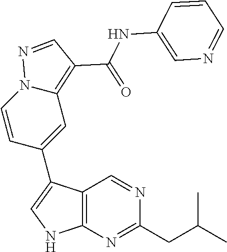 [SMILES: CC(C)Cc1ncc2c(-c3ccn4ncc(C(=O)Nc5cccnc5)c4c3)c[nH]c2n1] | 1593 |
| 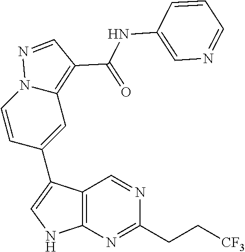 [SMILES: O=C(Nc1cccnc1)c1cnn2ccc(-c3c[nH]c4nc(CCC(F)(F)F)ncc34)cc12] | 1594 |
| 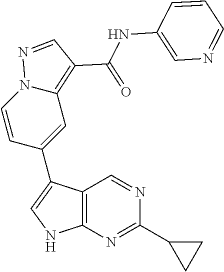 [SMILES: O=C(Nc1cccnc1)c1cnn2ccc(-c3c[nH]c4nc(C5CC5)ncc34)cc12] | 1595 |
| 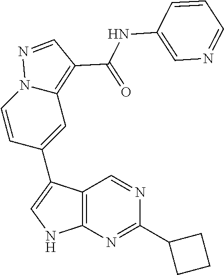 [SMILES: O=C(Nc1cccnc1)c1cnn2ccc(-c3c[nH]c4nc(C5CCC5)ncc34)cc12] | 1596 |
| 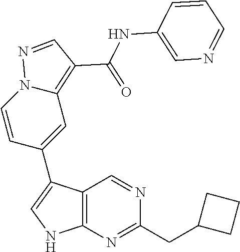 [SMILES: O=C(Nc1cccnc1)c1cnn2ccc(-c3c[nH]c4nc(CC5CCC5)ncc34)cc12] | 1597 |
| 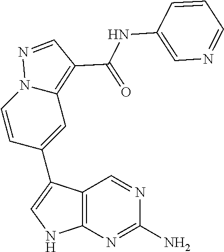 [SMILES: Nc1ncc2c(-c3ccn4ncc(C(=O)Nc5cccnc5)c4c3)c[nH]c2n1] | 1598 |
| 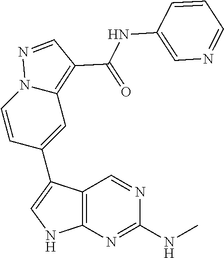 [SMILES: CNc1ncc2c(-c3ccn4ncc(C(=O)Nc5cccnc5)c4c3)c[nH]c2n1] | 1599 |
| 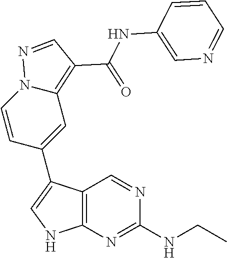 [SMILES: CCNc1ncc2c(-c3ccn4ncc(C(=O)Nc5cccnc5)c4c3)c[nH]c2n1] | 1600 |
| 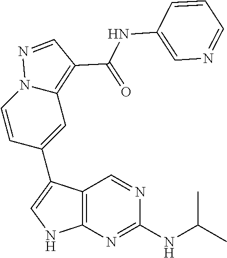 [SMILES: CC(C)Nc1ncc2c(-c3ccn4ncc(C(=O)Nc5cccnc5)c4c3)c[nH]c2n1] | 1601 |
| 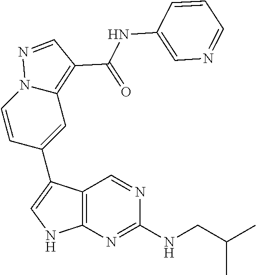 [SMILES: CC(C)CNc1ncc2c(-c3ccn4ncc(C(=O)Nc5cccnc5)c4c3)c[nH]c2n1] | 1602 |
| 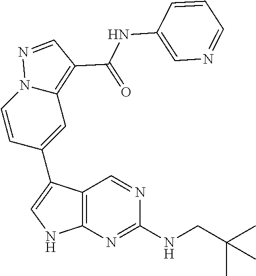 [SMILES: CC(C)(C)CNc1ncc2c(-c3ccn4ncc(C(=O)Nc5cccnc5)c4c3)c[nH]c2n1] | 1603 |
| 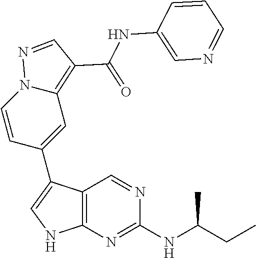 [SMILES: CC[C@H](C)Nc1ncc2c(-c3ccn4ncc(C(=O)Nc5cccnc5)c4c3)c[nH]c2n1] | 1604 |
| 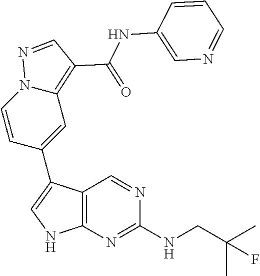 [SMILES: CC(C)(F)CNc1ncc2c(-c3ccn4ncc(C(=O)Nc5cccnc5)c4c3)c[nH]c2n1] | 1605 |
| 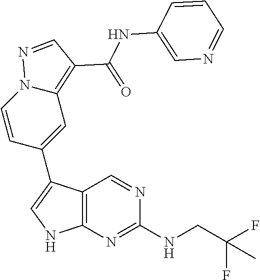 [SMILES: CC(F)(F)CNc1ncc2c(-c3ccn4ncc(C(=O)Nc5cccnc5)c4c3)c[nH]c2n1] | 1606 |
| 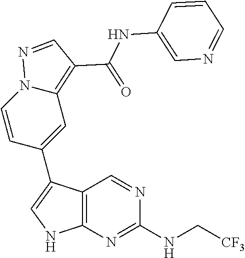 [SMILES: O=C(Nc1cccnc1)c1cnn2ccc(-c3c[nH]c4nc(NCC(F)(F)F)ncc34)cc12] | 1607 |
| 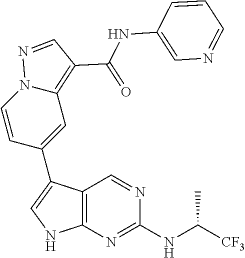 [SMILES: C[C@@H](Nc1ncc2c(-c3ccn4ncc(C(=O)Nc5cccnc5)c4c3)c[nH]c2n1)C(F)(F)F] | 1608 |
| 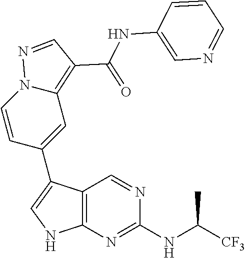 [SMILES: C[C@H](Nc1ncc2c(-c3ccn4ncc(C(=O)Nc5cccnc5)c4c3)c[nH]c2n1)C(F)(F)F] | 1609 |
| 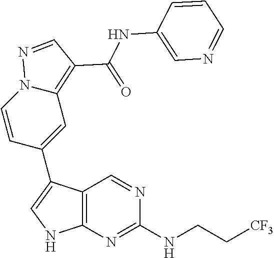 [SMILES: O=C(Nc1cccnc1)c1cnn2ccc(-c3c[nH]c4nc(NCCC(F)(F)F)ncc34)cc12] | 1610 |
| 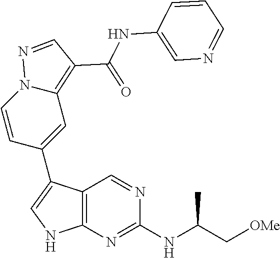 [SMILES: COC[C@H](C)Nc1ncc2c(-c3ccn4ncc(C(=O)Nc5cccnc5)c4c3)c[nH]c2n1] | 1611 |
| 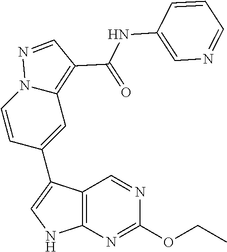 [SMILES: CCOc1ncc2c(-c3ccn4ncc(C(=O)Nc5cccnc5)c4c3)c[nH]c2n1] | 1612 |
| 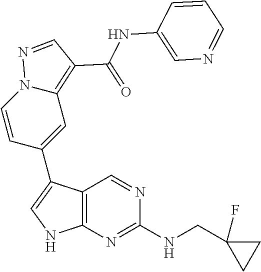 [SMILES: O=C(Nc1cccnc1)c1cnn2ccc(-c3c[nH]c4nc(NCC5(F)CC5)ncc34)cc12] | 1613 |
| 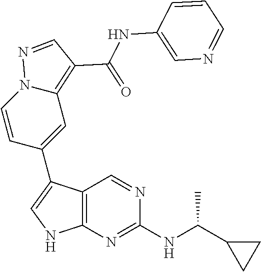 [SMILES: C[C@@H](Nc1ncc2c(-c3ccn4ncc(C(=O)Nc5cccnc5)c4c3)c[nH]c2n1)C1CC1] | 1614 |
| 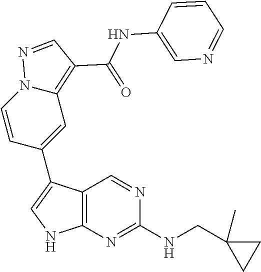 [SMILES: CC1(CNc2ncc3c(-c4ccn5ncc(C(=O)Nc6cccnc6)c5c4)c[nH]c3n2)CC1] | 1615 |
| 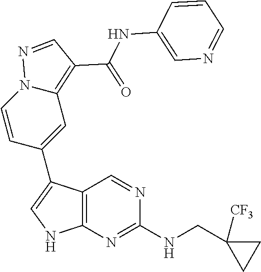 [SMILES: O=C(Nc1cccnc1)c1cnn2ccc(-c3c[nH]c4nc(NCC5(C(F)(F)F)CC5)ncc34)cc12] | 1616 |
| 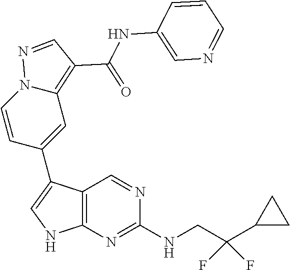 [SMILES: O=C(Nc1cccnc1)c1cnn2ccc(-c3c[nH]c4nc(NCC(F)(F)C5CC5)ncc34)cc12] | 1617 |
| 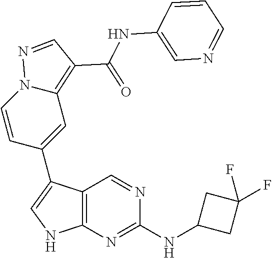 [SMILES: O=C(Nc1cccnc1)c1cnn2ccc(-c3c[nH]c4nc(NC5CC(F)(F)C5)ncc34)cc12] | 1618 |
| 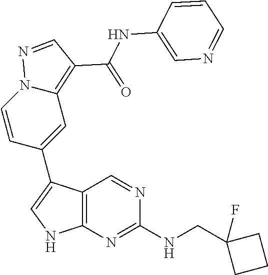 [SMILES: O=C(Nc1cccnc1)c1cnn2ccc(-c3c[nH]c4nc(NCC5(F)CCC5)ncc34)cc12] | 1619 |
| 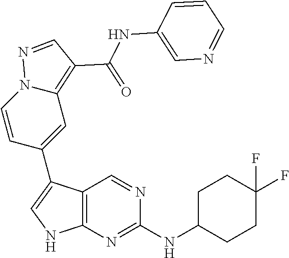 [SMILES: O=C(Nc1cccnc1)c1cnn2ccc(-c3c[nH]c4nc(NC5CCC(F)(F)CC5)ncc34)cc12] | 1620 |
| 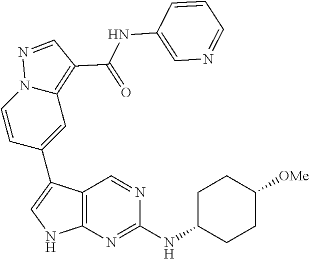 [SMILES: CO[C@H]1CC[C@@H](Nc2ncc3c(-c4ccn5ncc(C(=O)Nc6cccnc6)c5c4)c[nH]c3n2)CC1] | 1621 |
| 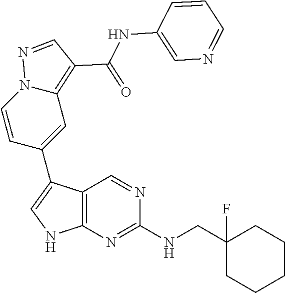 [SMILES: O=C(Nc1cccnc1)c1cnn2ccc(-c3c[nH]c4nc(NCC5(F)CCCCC5)ncc34)cc12] | 1622 |
| 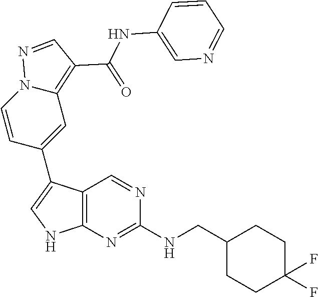 [SMILES: O=C(Nc1cccnc1)c1cnn2ccc(-c3c[nH]c4nc(NCC5CCC(F)(F)CC5)ncc34)cc12] | 1623 |
| 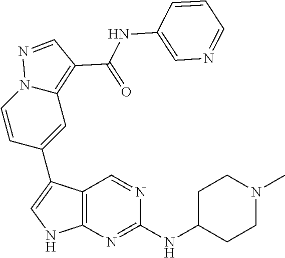 [SMILES: CN1CCC(Nc2ncc3c(-c4ccn5ncc(C(=O)Nc6cccnc6)c5c4)c[nH]c3n2)CC1] | 1624 |
| 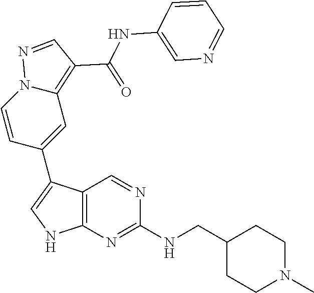 [SMILES: CN1CCC(CNc2ncc3c(-c4ccn5ncc(C(=O)Nc6cccnc6)c5c4)c[nH]c3n2)CC1] | 1625 |
| 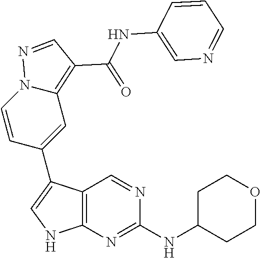 [SMILES: O=C(Nc1cccnc1)c1cnn2ccc(-c3c[nH]c4nc(NC5CCOCC5)ncc34)cc12] | 1626 |
| 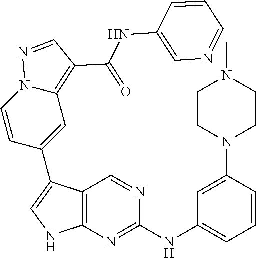 [SMILES: CN1CCN(c2cccc(Nc3ncc4c(-c5ccn6ncc(C(=O)Nc7cccnc7)c6c5)c[nH]c4n3)c2)CC1] | 1627 |
| 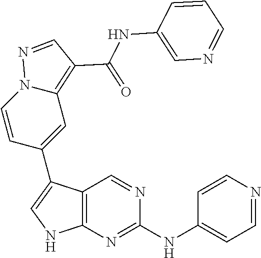 [SMILES: O=C(Nc1cccnc1)c1cnn2ccc(-c3c[nH]c4nc(Nc5ccncc5)ncc34)cc12] | 1628 |
| 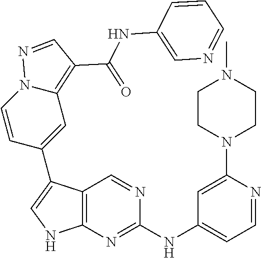 [SMILES: CN1CCN(c2cc(Nc3ncc4c(-c5ccn6ncc(C(=O)Nc7cccnc7)c6c5)c[nH]c4n3)ccn2)CC1] | 1629 |
| 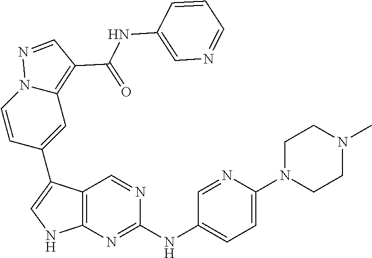 [SMILES: CN1CCN(c2ccc(Nc3ncc4c(-c5ccn6ncc(C(=O)Nc7cccnc7)c6c5)c[nH]c4n3)cn2)CC1] | 1630 |
| 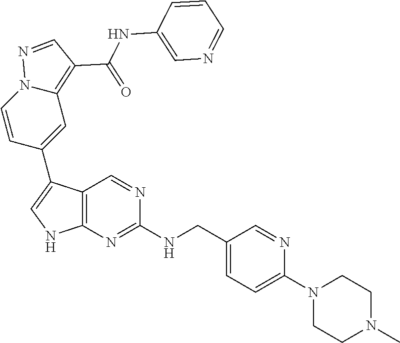 [SMILES: CN1CCN(c2ccc(CNc3ncc4c(-c5ccn6ncc(C(=O)Nc7cccnc7)c6c5)c[nH]c4n3)cn2)CC1] | 1631 |
| 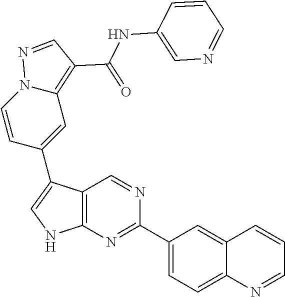 [SMILES: O=C(Nc1cccnc1)c1cnn2ccc(-c3c[nH]c4nc(-c5ccc6ncccc6c5)ncc34)cc12] | 1632 |
| 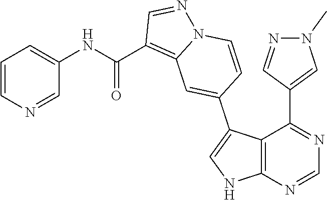 [SMILES: Cn1cc(-c2ncnc3[nH]cc(-c4ccn5ncc(C(=O)Nc6cccnc6)c5c4)c23)cn1] | 1633 |
| 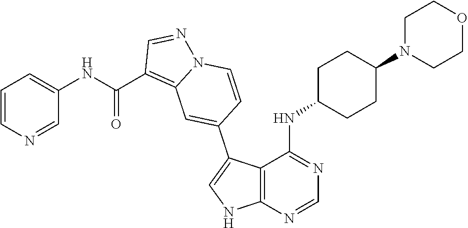 [SMILES: O=C(Nc1cccnc1)c1cnn2ccc(-c3c[nH]c4ncnc(N[C@H]5CC[C@H](N6CCOCC6)CC5)c34)cc12] | 1634 |
| 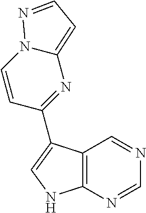 [SMILES: c1ncc2c(-c3ccn4nccc4n3)c[nH]c2n1] | 1635 |
| 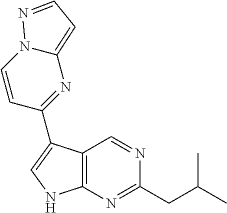 [SMILES: CC(C)Cc1ncc2c(-c3ccn4nccc4n3)c[nH]c2n1] | 1636 |
| 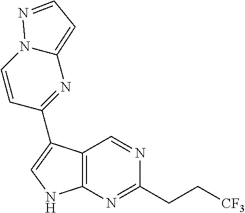 [SMILES: FC(F)(F)CCc1ncc2c(-c3ccn4nccc4n3)c[nH]c2n1] | 1637 |
| 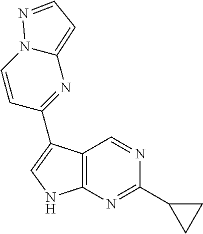 [SMILES: c1cc2nc(-c3c[nH]c4nc(C5CC5)ncc34)ccn2n1] | 1638 |
| 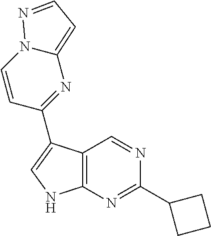 [SMILES: c1cc2nc(-c3c[nH]c4nc(C5CCC5)ncc34)ccn2n1] | 1639 |
| 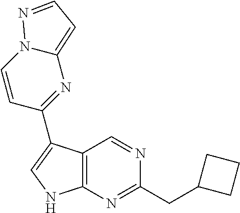 [SMILES: c1cc2nc(-c3c[nH]c4nc(CC5CCC5)ncc34)ccn2n1] | 1640 |
| 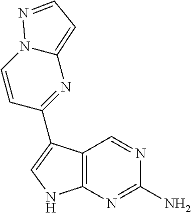 [SMILES: Nc1ncc2c(-c3ccn4nccc4n3)c[nH]c2n1] | 1641 |
| 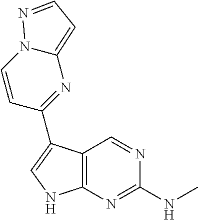 [SMILES: CNc1ncc2c(-c3ccn4nccc4n3)c[nH]c2n1] | 1642 |
| 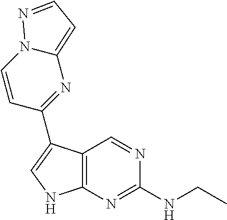 [SMILES: CCNc1ncc2c(-c3ccn4nccc4n3)c[nH]c2n1] | 1643 |
| 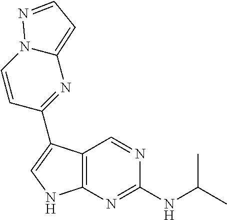 [SMILES: CC(C)Nc1ncc2c(-c3ccn4nccc4n3)c[nH]c2n1] | 1644 |
| 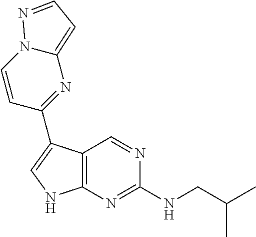 [SMILES: CC(C)CNc1ncc2c(-c3ccn4nccc4n3)c[nH]c2n1] | 1645 |
| 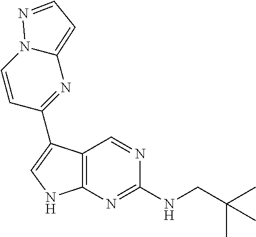 [SMILES: CC(C)(C)CNc1ncc2c(-c3ccn4nccc4n3)c[nH]c2n1] | 1646 |
| 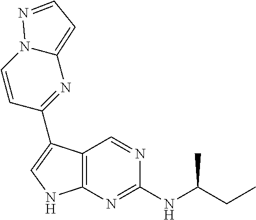 [SMILES: CC[C@H](C)Nc1ncc2c(-c3ccn4nccc4n3)c[nH]c2n1] | 1647 |
| 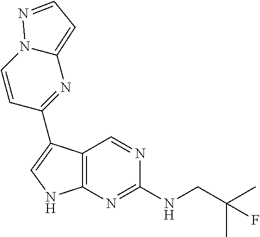 [SMILES: CC(C)(F)CNc1ncc2c(-c3ccn4nccc4n3)c[nH]c2n1] | 1648 |
| 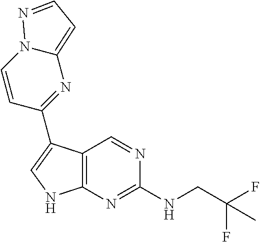 [SMILES: CC(F)(F)CNc1ncc2c(-c3ccn4nccc4n3)c[nH]c2n1] | 1649 |
| 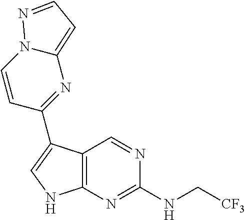 [SMILES: FC(F)(F)CNc1ncc2c(-c3ccn4nccc4n3)c[nH]c2n1] | 1650 |
| 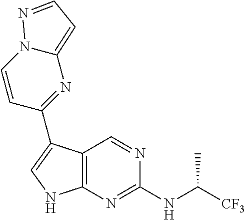 [SMILES: C[C@@H](Nc1ncc2c(-c3ccn4nccc4n3)c[nH]c2n1)C(F)(F)F] | 1651 |
| 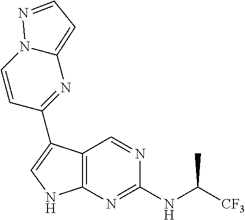 [SMILES: C[C@H](Nc1ncc2c(-c3ccn4nccc4n3)c[nH]c2n1)C(F)(F)F] | 1652 |
| 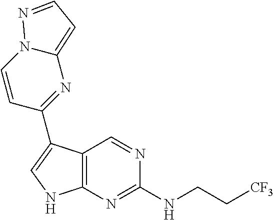 [SMILES: FC(F)(F)CCNc1ncc2c(-c3ccn4nccc4n3)c[nH]c2n1] | 1653 |
| 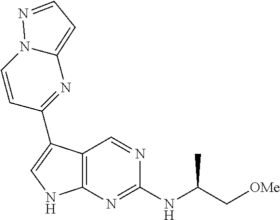 [SMILES: COC[C@H](C)Nc1ncc2c(-c3ccn4nccc4n3)c[nH]c2n1] | 1654 |
| 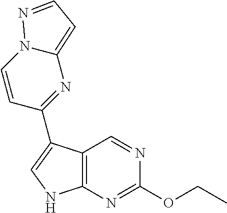 [SMILES: CCOc1ncc2c(-c3ccn4nccc4n3)c[nH]c2n1] | 1655 |
| 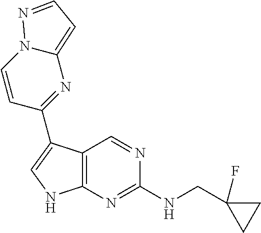 [SMILES: FC1(CNc2ncc3c(-c4ccn5nccc5n4)c[nH]c3n2)CC1] | 1656 |
| 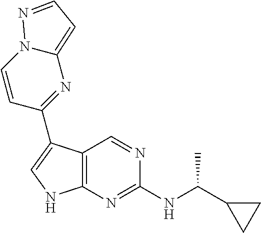 [SMILES: C[C@@H](Nc1ncc2c(-c3ccn4nccc4n3)c[nH]c2n1)C1CC1] | 1657 |
| 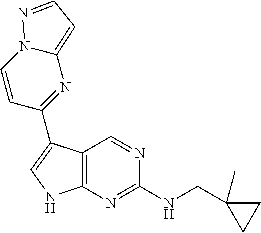 [SMILES: CC1(CNc2ncc3c(-c4ccn5nccc5n4)c[nH]c3n2)CC1] | 1658 |
| 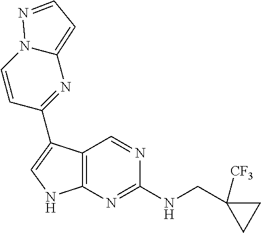 [SMILES: FC(F)(F)C1(CNc2ncc3c(-c4ccn5nccc5n4)c[nH]c3n2)CC1] | 1659 |
| 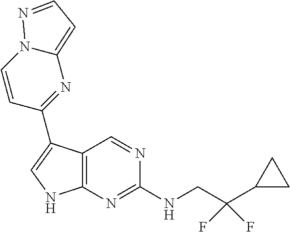 [SMILES: FC(F)(CNc1ncc2c(-c3ccn4nccc4n3)c[nH]c2n1)C1CC1] | 1660 |
| 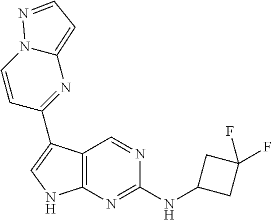 [SMILES: FC1(F)CC(Nc2ncc3c(-c4ccn5nccc5n4)c[nH]c3n2)C1] | 1661 |
| 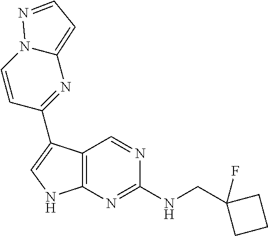 [SMILES: FC1(CNc2ncc3c(-c4ccn5nccc5n4)c[nH]c3n2)CCC1] | 1662 |
| 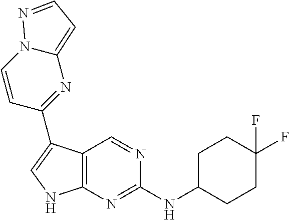 [SMILES: FC1(F)CCC(Nc2ncc3c(-c4ccn5nccc5n4)c[nH]c3n2)CC1] | 1663 |
| 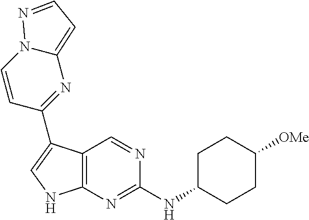 [SMILES: CO[C@H]1CC[C@@H](Nc2ncc3c(-c4ccn5nccc5n4)c[nH]c3n2)CC1] | 1664 |
| 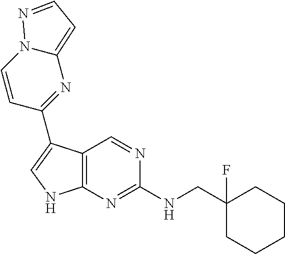 [SMILES: FC1(CNc2ncc3c(-c4ccn5nccc5n4)c[nH]c3n2)CCCCC1] | 1665 |
| 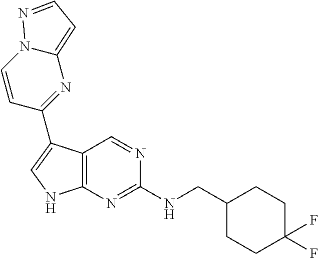 [SMILES: FC1(F)CCC(CNc2ncc3c(-c4ccn5nccc5n4)c[nH]c3n2)CC1] | 1666 |
| 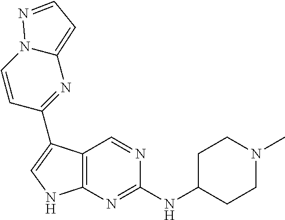 [SMILES: CN1CCC(Nc2ncc3c(-c4ccn5nccc5n4)c[nH]c3n2)CC1] | 1667 |
| 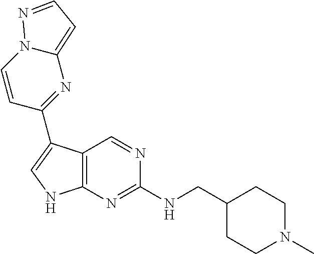 [SMILES: CN1CCC(CNc2ncc3c(-c4ccn5nccc5n4)c[nH]c3n2)CC1] | 1668 |
| 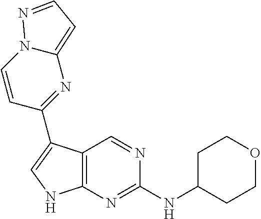 [SMILES: c1cc2nc(-c3c[nH]c4nc(NC5CCOCC5)ncc34)ccn2n1] | 1669 |
| 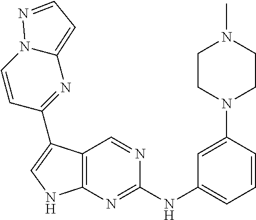 [SMILES: CN1CCN(c2cccc(Nc3ncc4c(-c5ccn6nccc6n5)c[nH]c4n3)c2)CC1] | 1670 |
| 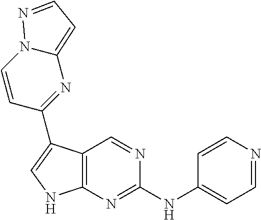 [SMILES: c1cc(Nc2ncc3c(-c4ccn5nccc5n4)c[nH]c3n2)ccn1] | 1671 |
| 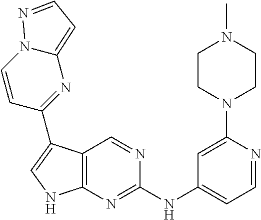 [SMILES: CN1CCN(c2cc(Nc3ncc4c(-c5ccn6nccc6n5)c[nH]c4n3)ccn2)CC1] | 1672 |
| 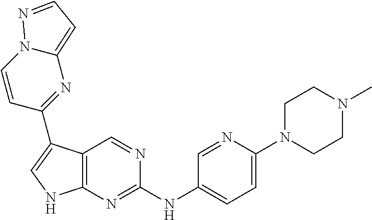 [SMILES: CN1CCN(c2ccc(Nc3ncc4c(-c5ccn6nccc6n5)c[nH]c4n3)cn2)CC1] | 1673 |
| 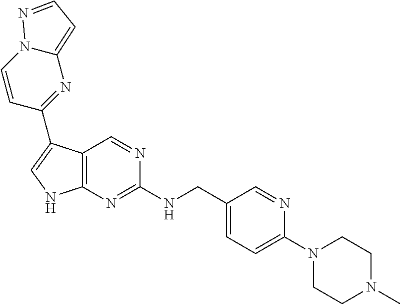 [SMILES: CN1CCN(c2ccc(CNc3ncc4c(-c5ccn6nccc6n5)c[nH]c4n3)cn2)CC1] | 1674 |
| 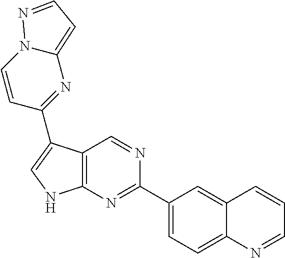 [SMILES: c1cnc2ccc(-c3ncc4c(-c5ccn6nccc6n5)c[nH]c4n3)cc2c1] | 1675 |
| 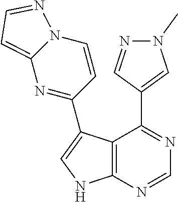 [SMILES: Cn1cc(-c2ncnc3[nH]cc(-c4ccn5nccc5n4)c23)cn1] | 1676 |
| 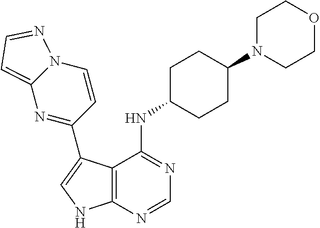 [SMILES: c1nc(N[C@H]2CC[C@H](N3CCOCC3)CC2)c2c(-c3ccn4nccc4n3)c[nH]c2n1] | 1677 |
| 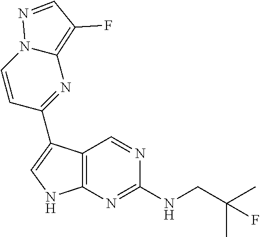 [SMILES: CC(C)(F)CNc1ncc2c(-c3ccn4ncc(F)c4n3)c[nH]c2n1] | 1678 |
| 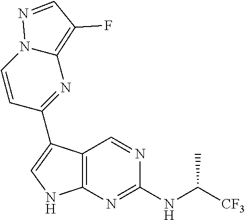 [SMILES: C[C@@H](Nc1ncc2c(-c3ccn4ncc(F)c4n3)c[nH]c2n1)C(F)(F)F] | 1679 |
| 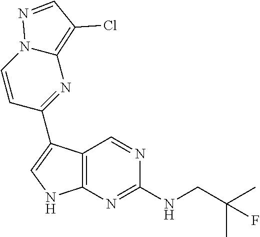 [SMILES: CC(C)(F)CNc1ncc2c(-c3ccn4ncc(Cl)c4n3)c[nH]c2n1] | 1680 |
| 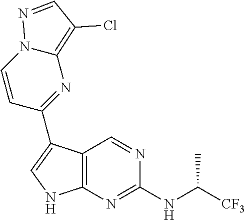 [SMILES: C[C@@H](Nc1ncc2c(-c3ccn4ncc(Cl)c4n3)c[nH]c2n1)C(F)(F)F] | 1681 |
| 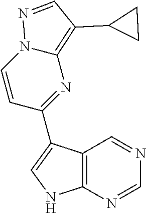 [SMILES: c1ncc2c(-c3ccn4ncc(C5CC5)c4n3)c[nH]c2n1] | 1682 |
| 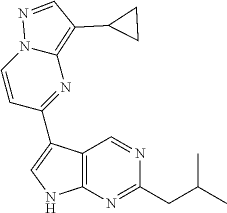 [SMILES: CC(C)Cc1ncc2c(-c3ccn4ncc(C5CC5)c4n3)c[nH]c2n1] | 1683 |
| 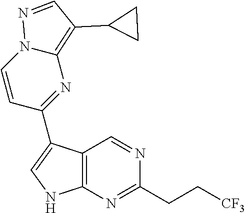 [SMILES: FC(F)(F)CCc1ncc2c(-c3ccn4ncc(C5CC5)c4n3)c[nH]c2n1] | 1684 |
| 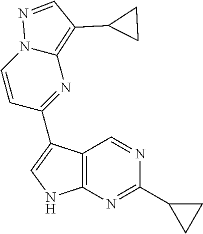 [SMILES: c1cn2ncc(C3CC3)c2nc1-c1c[nH]c2nc(C3CC3)ncc12] | 1685 |
| 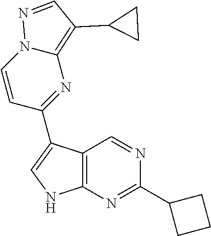 [SMILES: c1cn2ncc(C3CC3)c2nc1-c1c[nH]c2nc(C3CCC3)ncc12] | 1686 |
| 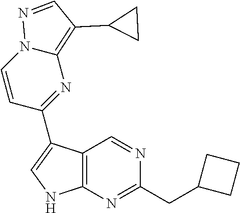 [SMILES: c1cn2ncc(C3CC3)c2nc1-c1c[nH]c2nc(CC3CCC3)ncc12] | 1687 |
| 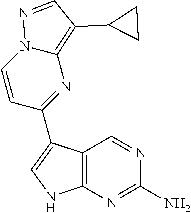 [SMILES: Nc1ncc2c(-c3ccn4ncc(C5CC5)c4n3)c[nH]c2n1] | 1688 |
| 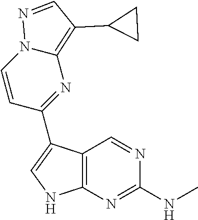 [SMILES: CNc1ncc2c(-c3ccn4ncc(C5CC5)c4n3)c[nH]c2n1] | 1689 |
| 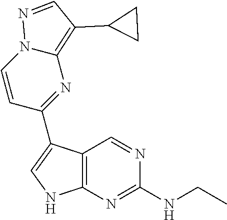 [SMILES: CCNc1ncc2c(-c3ccn4ncc(C5CC5)c4n3)c[nH]c2n1] | 1690 |
| 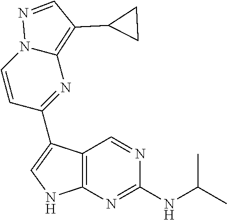 [SMILES: CC(C)Nc1ncc2c(-c3ccn4ncc(C5CC5)c4n3)c[nH]c2n1] | 1691 |
| 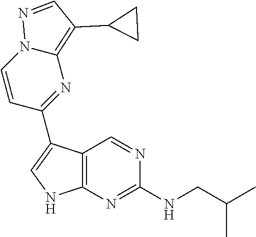 [SMILES: CC(C)CNc1ncc2c(-c3ccn4ncc(C5CC5)c4n3)c[nH]c2n1] | 1692 |
| 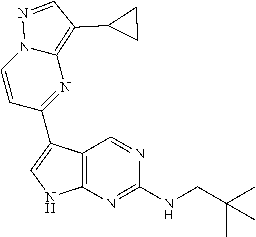 [SMILES: CC(C)(C)CNc1ncc2c(-c3ccn4ncc(C5CC5)c4n3)c[nH]c2n1] | 1693 |
| 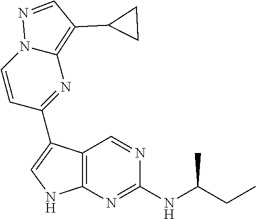 [SMILES: CC[C@H](C)Nc1ncc2c(-c3ccn4ncc(C5CC5)c4n3)c[nH]c2n1] | 1694 |
| 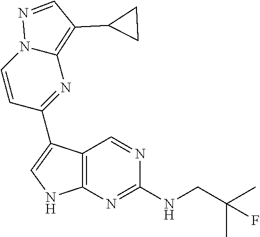 [SMILES: CC(C)(F)CNc1ncc2c(-c3ccn4ncc(C5CC5)c4n3)c[nH]c2n1] | 1695 |
| 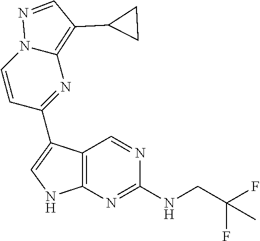 [SMILES: CC(F)(F)CNc1ncc2c(-c3ccn4ncc(C5CC5)c4n3)c[nH]c2n1] | 1696 |
| 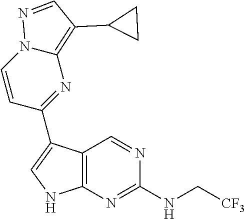 [SMILES: FC(F)(F)CNc1ncc2c(-c3ccn4ncc(C5CC5)c4n3)c[nH]c2n1] | 1697 |
| 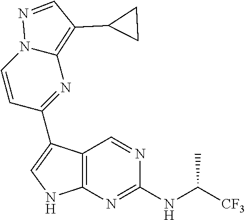 [SMILES: C[C@@H](Nc1ncc2c(-c3ccn4ncc(C5CC5)c4n3)c[nH]c2n1)C(F)(F)F] | 1698 |
| 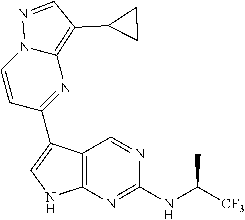 [SMILES: C[C@H](Nc1ncc2c(-c3ccn4ncc(C5CC5)c4n3)c[nH]c2n1)C(F)(F)F] | 1699 |
| 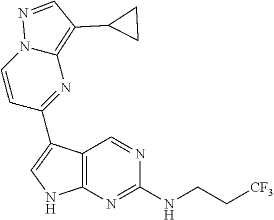 [SMILES: FC(F)(F)CCNc1ncc2c(-c3ccn4ncc(C5CC5)c4n3)c[nH]c2n1] | 1700 |
| 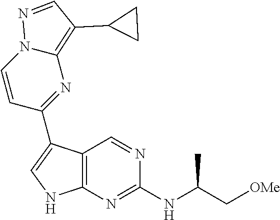 [SMILES: COC[C@H](C)Nc1ncc2c(-c3ccn4ncc(C5CC5)c4n3)c[nH]c2n1] | 1701 |
| 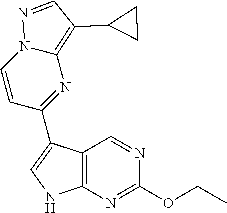 [SMILES: CCOc1ncc2c(-c3ccn4ncc(C5CC5)c4n3)c[nH]c2n1] | 1702 |
| 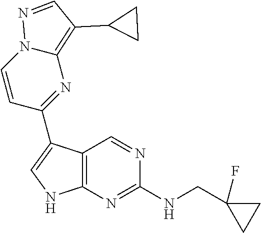 [SMILES: FC1(CNc2ncc3c(-c4ccn5ncc(C6CC6)c5n4)c[nH]c3n2)CC1] | 1703 |
| 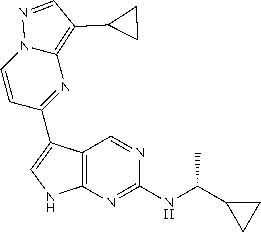 [SMILES: C[C@@H](Nc1ncc2c(-c3ccn4ncc(C5CC5)c4n3)c[nH]c2n1)C1CC1] | 1704 |
| 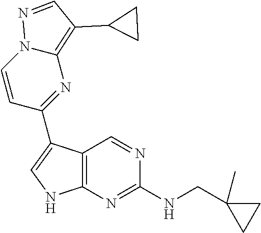 [SMILES: CC1(CNc2ncc3c(-c4ccn5ncc(C6CC6)c5n4)c[nH]c3n2)CC1] | 1705 |
| 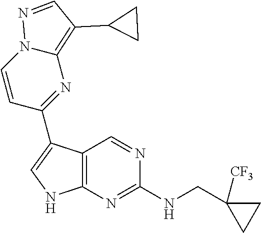 [SMILES: FC(F)(F)C1(CNc2ncc3c(-c4ccn5ncc(C6CC6)c5n4)c[nH]c3n2)CC1] | 1706 |
| 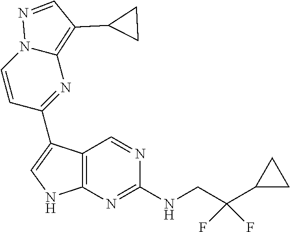 [SMILES: FC(F)(CNc1ncc2c(-c3ccn4ncc(C5CC5)c4n3)c[nH]c2n1)C1CC1] | 1707 |
| 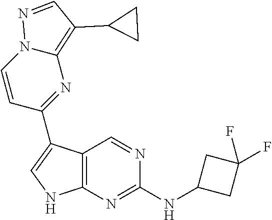 [SMILES: FC1(F)CC(Nc2ncc3c(-c4ccn5ncc(C6CC6)c5n4)c[nH]c3n2)C1] | 1708 |
| 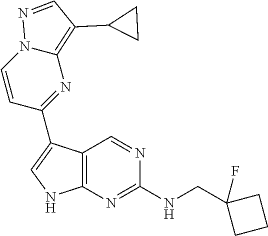 [SMILES: FC1(CNc2ncc3c(-c4ccn5ncc(C6CC6)c5n4)c[nH]c3n2)CCC1] | 1709 |
| 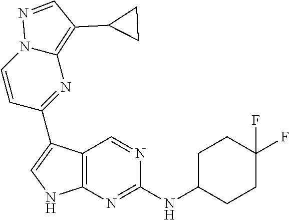 [SMILES: FC1(F)CCC(Nc2ncc3c(-c4ccn5ncc(C6CC6)c5n4)c[nH]c3n2)CC1] | 1710 |
| 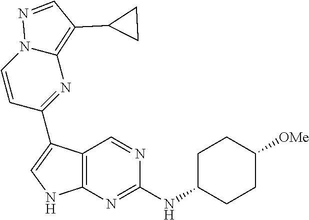 [SMILES: CO[C@H]1CC[C@@H](Nc2ncc3c(-c4ccn5ncc(C6CC6)c5n4)c[nH]c3n2)CC1] | 1711 |
| 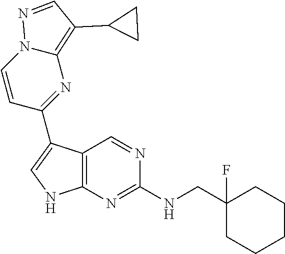 [SMILES: FC1(CNc2ncc3c(-c4ccn5ncc(C6CC6)c5n4)c[nH]c3n2)CCCCC1] | 1712 |
| 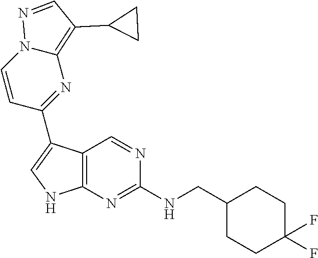 [SMILES: FC1(F)CCC(CNc2ncc3c(-c4ccn5ncc(C6CC6)c5n4)c[nH]c3n2)CC1] | 1713 |
| 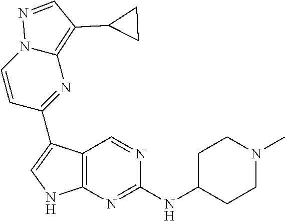 [SMILES: CN1CCC(Nc2ncc3c(-c4ccn5ncc(C6CC6)c5n4)c[nH]c3n2)CC1] | 1714 |
| 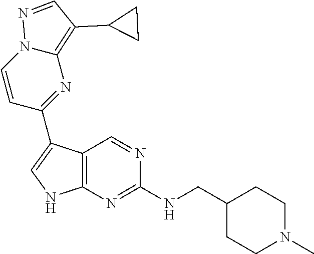 [SMILES: CN1CCC(CNc2ncc3c(-c4ccn5ncc(C6CC6)c5n4)c[nH]c3n2)CC1] | 1715 |
| 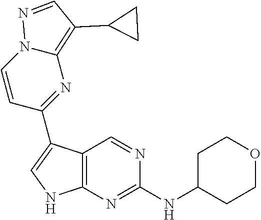 [SMILES: c1cn2ncc(C3CC3)c2nc1-c1c[nH]c2nc(NC3CCOCC3)ncc12] | 1716 |
| 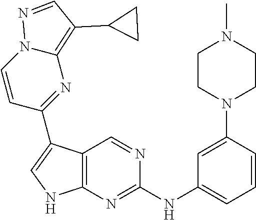 [SMILES: CN1CCN(c2cccc(Nc3ncc4c(-c5ccn6ncc(C7CC7)c6n5)c[nH]c4n3)c2)CC1] | 1717 |
| 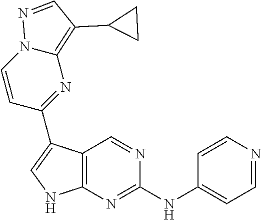 [SMILES: c1cc(Nc2ncc3c(-c4ccn5ncc(C6CC6)c5n4)c[nH]c3n2)ccn1] | 1718 |
| 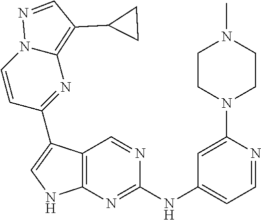 [SMILES: CN1CCN(c2cc(Nc3ncc4c(-c5ccn6ncc(C7CC7)c6n5)c[nH]c4n3)ccn2)CC1] | 1719 |
| 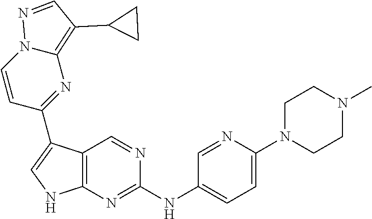 [SMILES: CN1CCN(c2ccc(Nc3ncc4c(-c5ccn6ncc(C7CC7)c6n5)c[nH]c4n3)cn2)CC1] | 1720 |
| 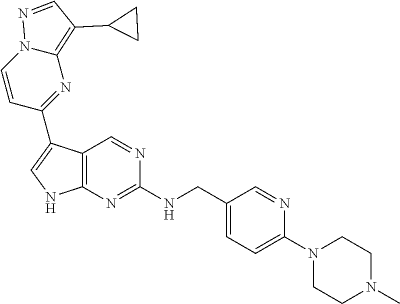 [SMILES: CN1CCN(c2ccc(CNc3ncc4c(-c5ccn6ncc(C7CC7)c6n5)c[nH]c4n3)cn2)CC1] | 1721 |
| 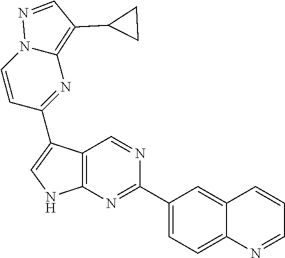 [SMILES: c1cnc2ccc(-c3ncc4c(-c5ccn6ncc(C7CC7)c6n5)c[nH]c4n3)cc2c1] | 1722 |
| 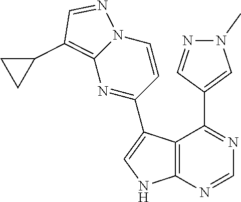 [SMILES: Cn1cc(-c2ncnc3[nH]cc(-c4ccn5ncc(C6CC6)c5n4)c23)cn1] | 1723 |
| 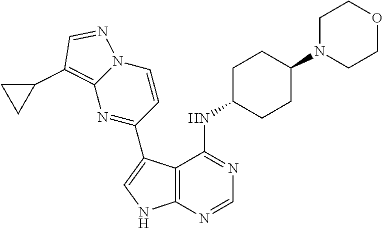 [SMILES: c1nc(N[C@H]2CC[C@H](N3CCOCC3)CC2)c2c(-c3ccn4ncc(C5CC5)c4n3)c[nH]c2n1] | 1724 |
| 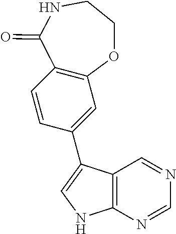 [SMILES: O=C1NCCOc2cc(-c3c[nH]c4ncncc34)ccc21] | 1725 |
| 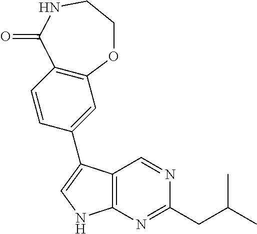 [SMILES: CC(C)Cc1ncc2c(-c3ccc4c(c3)OCCNC4=O)c[nH]c2n1] | 1726 |
| 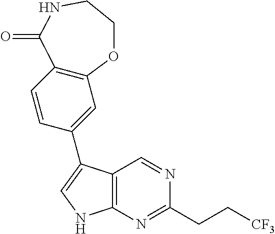 [SMILES: O=C1NCCOc2cc(-c3c[nH]c4nc(CCC(F)(F)F)ncc34)ccc21] | 1727 |
| 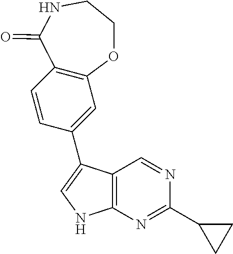 [SMILES: O=C1NCCOc2cc(-c3c[nH]c4nc(C5CC5)ncc34)ccc21] | 1728 |
| 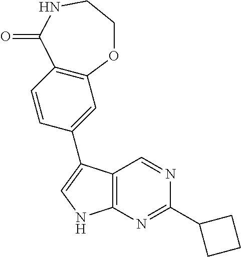 [SMILES: O=C1NCCOc2cc(-c3c[nH]c4nc(C5CCC5)ncc34)ccc21] | 1729 |
| 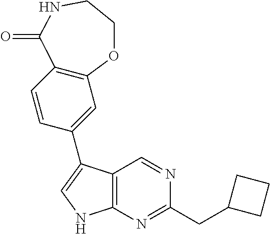 [SMILES: O=C1NCCOc2cc(-c3c[nH]c4nc(CC5CCC5)ncc34)ccc21] | 1730 |
| 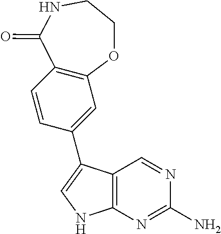 [SMILES: Nc1ncc2c(-c3ccc4c(c3)OCCNC4=O)c[nH]c2n1] | 1731 |
| 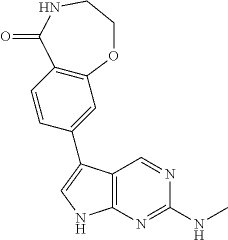 [SMILES: CNc1ncc2c(-c3ccc4c(c3)OCCNC4=O)c[nH]c2n1] | 1732 |
| 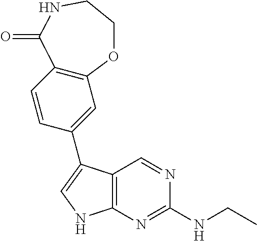 [SMILES: CCNc1ncc2c(-c3ccc4c(c3)OCCNC4=O)c[nH]c2n1] | 1733 |
| 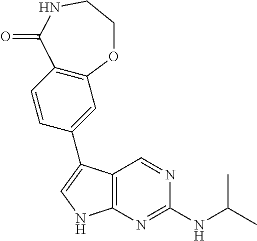 [SMILES: CC(C)Nc1ncc2c(-c3ccc4c(c3)OCCNC4=O)c[nH]c2n1] | 1734 |
| 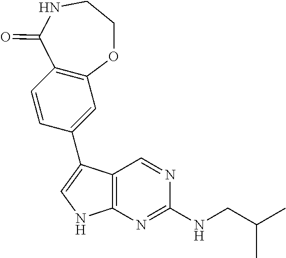 [SMILES: CC(C)CNc1ncc2c(-c3ccc4c(c3)OCCNC4=O)c[nH]c2n1] | 1735 |
| 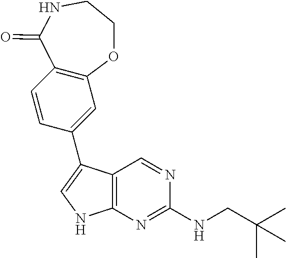 [SMILES: CC(C)(C)CNc1ncc2c(-c3ccc4c(c3)OCCNC4=O)c[nH]c2n1] | 1736 |
| 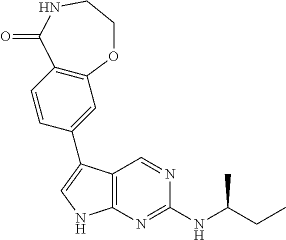 [SMILES: CC[C@H](C)Nc1ncc2c(-c3ccc4c(c3)OCCNC4=O)c[nH]c2n1] | 1737 |
| 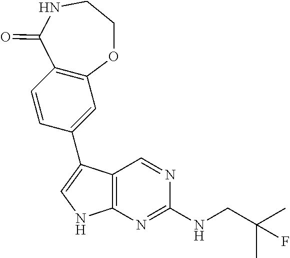 [SMILES: CC(C)(F)CNc1ncc2c(-c3ccc4c(c3)OCCNC4=O)c[nH]c2n1] | 1738 |
| 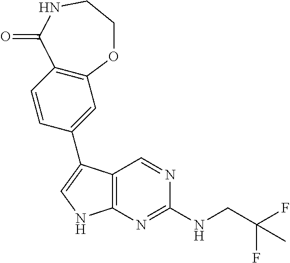 [SMILES: CC(F)(F)CNc1ncc2c(-c3ccc4c(c3)OCCNC4=O)c[nH]c2n1] | 1739 |
| 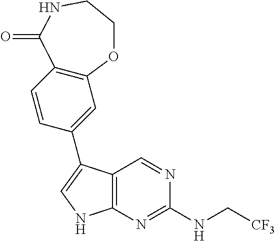 [SMILES: O=C1NCCOc2cc(-c3c[nH]c4nc(NCC(F)(F)F)ncc34)ccc21] | 1740 |
| 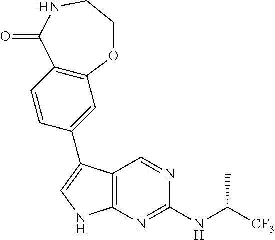 [SMILES: C[C@@H](Nc1ncc2c(-c3ccc4c(c3)OCCNC4=O)c[nH]c2n1)C(F)(F)F] | 1741 |
| 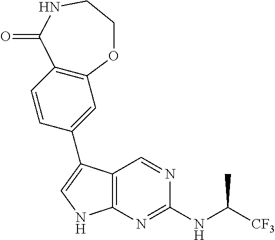 [SMILES: C[C@H](Nc1ncc2c(-c3ccc4c(c3)OCCNC4=O)c[nH]c2n1)C(F)(F)F] | 1742 |
| 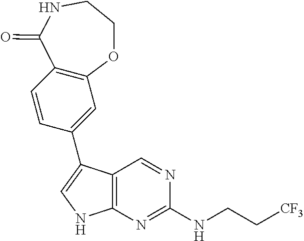 [SMILES: O=C1NCCOc2cc(-c3c[nH]c4nc(NCCC(F)(F)F)ncc34)ccc21] | 1743 |
| 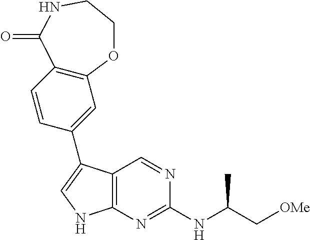 [SMILES: COC[C@H](C)Nc1ncc2c(-c3ccc4c(c3)OCCNC4=O)c[nH]c2n1] | 1744 |
| 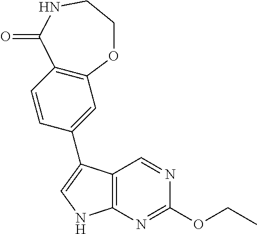 [SMILES: CCOc1ncc2c(-c3ccc4c(c3)OCCNC4=O)c[nH]c2n1] | 1745 |
| 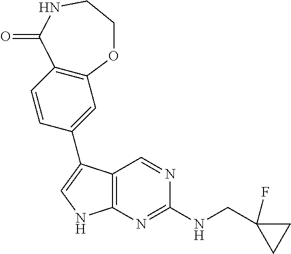 [SMILES: O=C1NCCOc2cc(-c3c[nH]c4nc(NCC5(F)CC5)ncc34)ccc21] | 1746 |
| 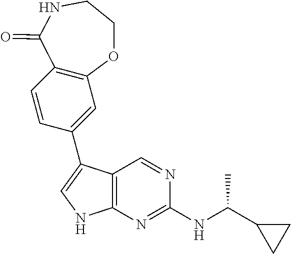 [SMILES: C[C@@H](Nc1ncc2c(-c3ccc4c(c3)OCCNC4=O)c[nH]c2n1)C1CC1] | 1747 |
| 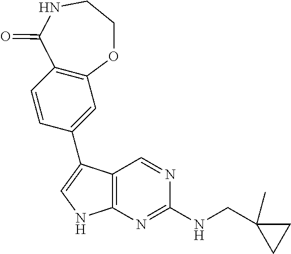 [SMILES: CC1(CNc2ncc3c(-c4ccc5c(c4)OCCNC5=O)c[nH]c3n2)CC1] | 1748 |
| 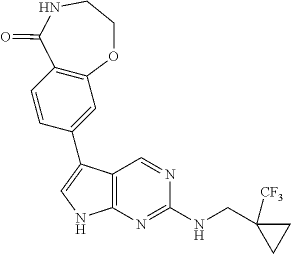 [SMILES: O=C1NCCOc2cc(-c3c[nH]c4nc(NCC5(C(F)(F)F)CC5)ncc34)ccc21] | 1749 |
| 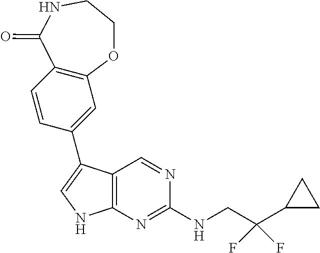 [SMILES: O=C1NCCOc2cc(-c3c[nH]c4nc(NCC(F)(F)C5CC5)ncc34)ccc21] | 1750 |
| 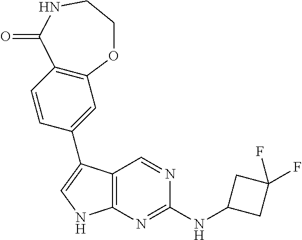 [SMILES: O=C1NCCOc2cc(-c3c[nH]c4nc(NC5CC(F)(F)C5)ncc34)ccc21] | 1751 |
| 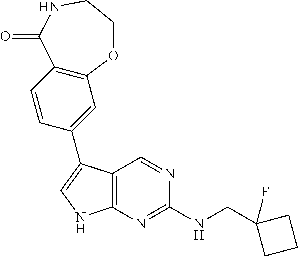 [SMILES: O=C1NCCOc2cc(-c3c[nH]c4nc(NCC5(F)CCC5)ncc34)ccc21] | 1752 |
| 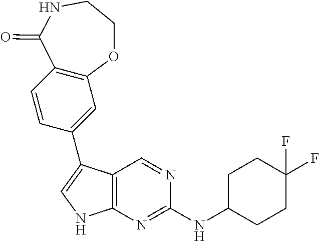 [SMILES: O=C1NCCOc2cc(-c3c[nH]c4nc(NC5CCC(F)(F)CC5)ncc34)ccc21] | 1753 |
| 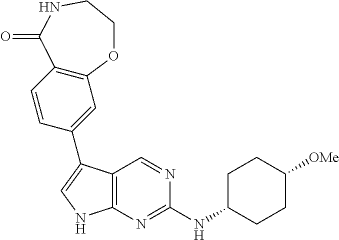 [SMILES: CO[C@H]1CC[C@@H](Nc2ncc3c(-c4ccc5c(c4)OCCNC5=O)c[nH]c3n2)CC1] | 1754 |
| 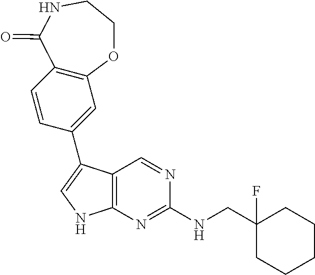 [SMILES: O=C1NCCOc2cc(-c3c[nH]c4nc(NCC5(F)CCCCC5)ncc34)ccc21] | 1755 |
| 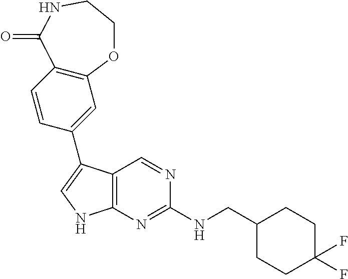 [SMILES: O=C1NCCOc2cc(-c3c[nH]c4nc(NCC5CCC(F)(F)CC5)ncc34)ccc21] | 1756 |
| 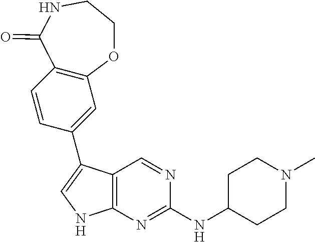 [SMILES: CN1CCC(Nc2ncc3c(-c4ccc5c(c4)OCCNC5=O)c[nH]c3n2)CC1] | 1757 |
| 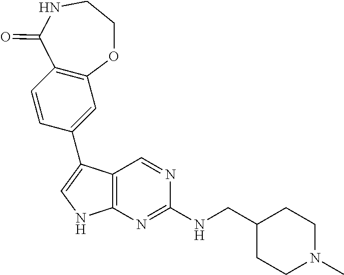 [SMILES: CN1CCC(CNc2ncc3c(-c4ccc5c(c4)OCCNC5=O)c[nH]c3n2)CC1] | 1758 |
| 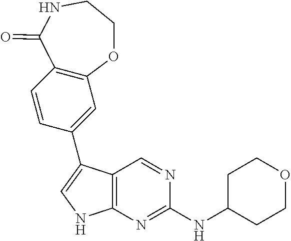 [SMILES: O=C1NCCOc2cc(-c3c[nH]c4nc(NC5CCOCC5)ncc34)ccc21] | 1759 |
| 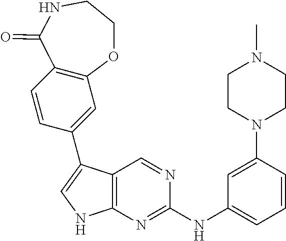 [SMILES: CN1CCN(c2cccc(Nc3ncc4c(-c5ccc6c(c5)OCCNC6=O)c[nH]c4n3)c2)CC1] | 1760 |
| 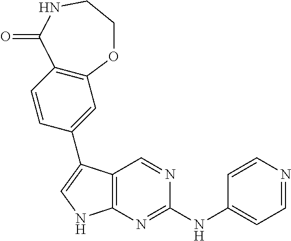 [SMILES: O=C1NCCOc2cc(-c3c[nH]c4nc(Nc5ccncc5)ncc34)ccc21] | 1761 |
| 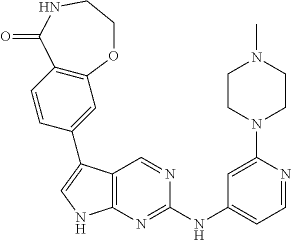 [SMILES: CN1CCN(c2cc(Nc3ncc4c(-c5ccc6c(c5)OCCNC6=O)c[nH]c4n3)ccn2)CC1] | 1762 |
| 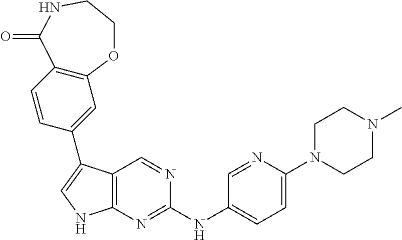 [SMILES: CN1CCN(c2ccc(Nc3ncc4c(-c5ccc6c(c5)OCCNC6=O)c[nH]c4n3)cn2)CC1] | 1763 |
| 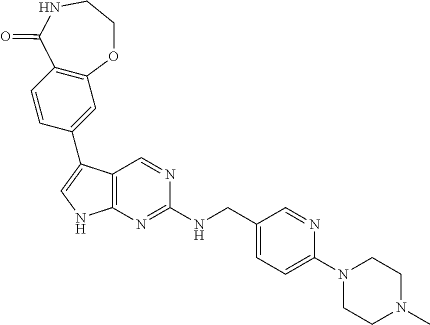 [SMILES: CN1CCN(c2ccc(CNc3ncc4c(-c5ccc6c(c5)OCCNC6=O)c[nH]c4n3)cn2)CC1] | 1764 |
| 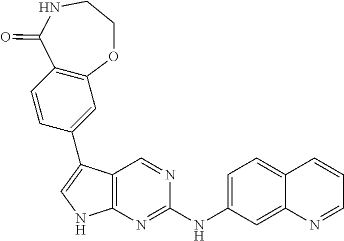 [SMILES: O=C1NCCOc2cc(-c3c[nH]c4nc(Nc5ccc6cccnc6c5)ncc34)ccc21] | 1765 |
| 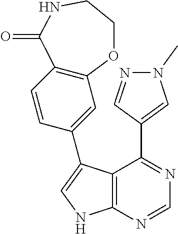 [SMILES: Cn1cc(-c2ncnc3[nH]cc(-c4ccc5c(c4)OCCNC5=O)c23)cn1] | 1766 |
| 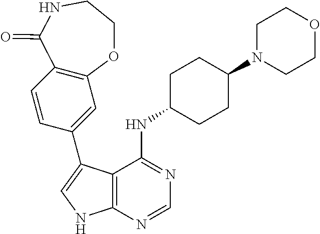 [SMILES: O=C1NCCOc2cc(-c3c[nH]c4ncnc(N[C@H]5CC[C@H](N6CCOCC6)CC5)c34)ccc21] | 1767 |
| 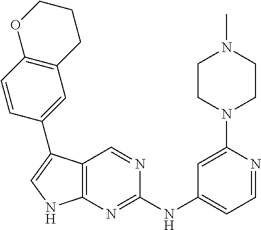 [SMILES: CN1CCN(c2cc(Nc3ncc4c(-c5ccc6c(c5)CCCO6)c[nH]c4n3)ccn2)CC1] | 1768 |
| 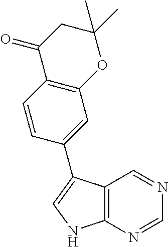 [SMILES: CC1(C)CC(=O)c2ccc(-c3c[nH]c4ncncc34)cc2O1] | 1769 |
| 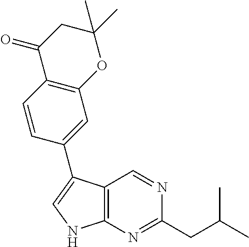 [SMILES: CC(C)Cc1ncc2c(-c3ccc4c(c3)OC(C)(C)CC4=O)c[nH]c2n1] | 1770 |
| 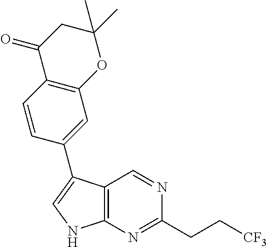 [SMILES: CC1(C)CC(=O)c2ccc(-c3c[nH]c4nc(CCC(F)(F)F)ncc34)cc2O1] | 1771 |
| 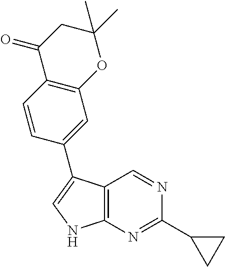 [SMILES: CC1(C)CC(=O)c2ccc(-c3c[nH]c4nc(C5CC5)ncc34)cc2O1] | 1772 |
| 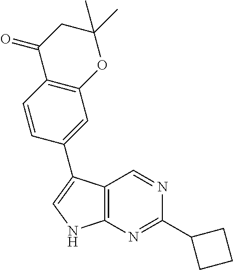 [SMILES: CC1(C)CC(=O)c2ccc(-c3c[nH]c4nc(C5CCC5)ncc34)cc2O1] | 1773 |
| 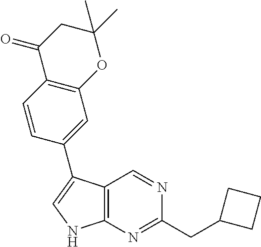 [SMILES: CC1(C)CC(=O)c2ccc(-c3c[nH]c4nc(CC5CCC5)ncc34)cc2O1] | 1774 |
| 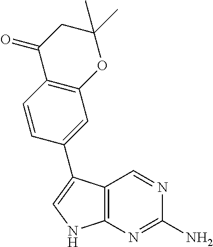 [SMILES: CC1(C)CC(=O)c2ccc(-c3c[nH]c4nc(N)ncc34)cc2O1] | 1775 |
| 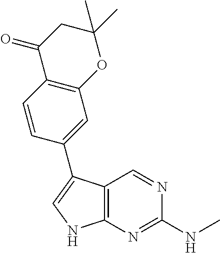 [SMILES: CNc1ncc2c(-c3ccc4c(c3)OC(C)(C)CC4=O)c[nH]c2n1] | 1776 |
| 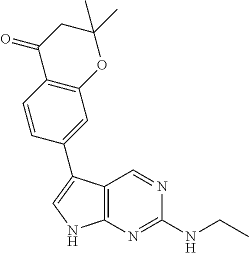 [SMILES: CCNc1ncc2c(-c3ccc4c(c3)OC(C)(C)CC4=O)c[nH]c2n1] | 1777 |
| 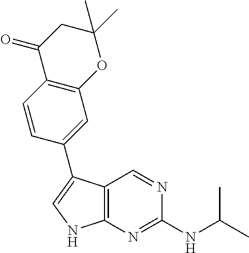 [SMILES: CC(C)Nc1ncc2c(-c3ccc4c(c3)OC(C)(C)CC4=O)c[nH]c2n1] | 1778 |
| 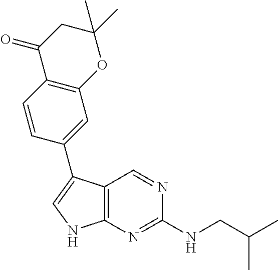 [SMILES: CC(C)CNc1ncc2c(-c3ccc4c(c3)OC(C)(C)CC4=O)c[nH]c2n1] | 1779 |
| 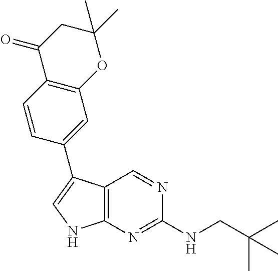 [SMILES: CC(C)(C)CNc1ncc2c(-c3ccc4c(c3)OC(C)(C)CC4=O)c[nH]c2n1] | 1780 |
| 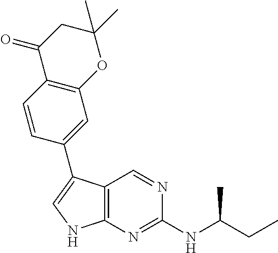 [SMILES: CC[C@H](C)Nc1ncc2c(-c3ccc4c(c3)OC(C)(C)CC4=O)c[nH]c2n1] | 1781 |
| 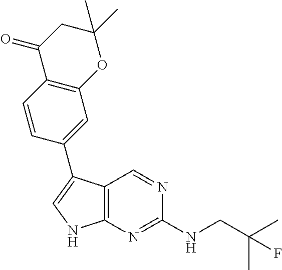 [SMILES: CC(C)(F)CNc1ncc2c(-c3ccc4c(c3)OC(C)(C)CC4=O)c[nH]c2n1] | 1782 |
| 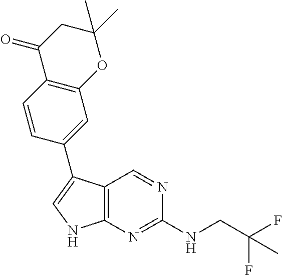 [SMILES: CC(F)(F)CNc1ncc2c(-c3ccc4c(c3)OC(C)(C)CC4=O)c[nH]c2n1] | 1783 |
| 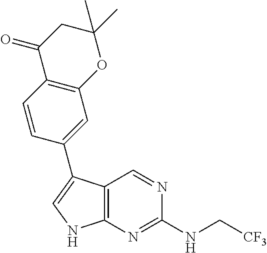 [SMILES: CC1(C)CC(=O)c2ccc(-c3c[nH]c4nc(NCC(F)(F)F)ncc34)cc2O1] | 1784 |
| 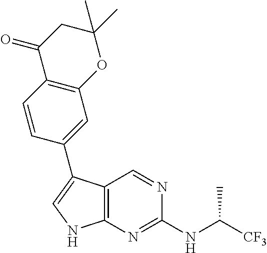 [SMILES: C[C@@H](Nc1ncc2c(-c3ccc4c(c3)OC(C)(C)CC4=O)c[nH]c2n1)C(F)(F)F] | 1785 |
| 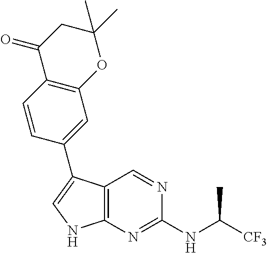 [SMILES: C[C@H](Nc1ncc2c(-c3ccc4c(c3)OC(C)(C)CC4=O)c[nH]c2n1)C(F)(F)F] | 1786 |
| 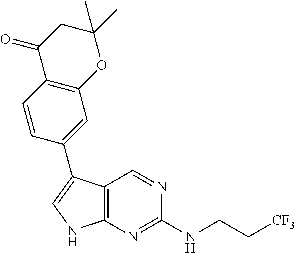 [SMILES: CC1(C)CC(=O)c2ccc(-c3c[nH]c4nc(NCCC(F)(F)F)ncc34)cc2O1] | 1787 |
| 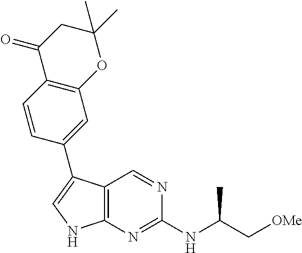 [SMILES: COC[C@H](C)Nc1ncc2c(-c3ccc4c(c3)OC(C)(C)CC4=O)c[nH]c2n1] | 1788 |
| 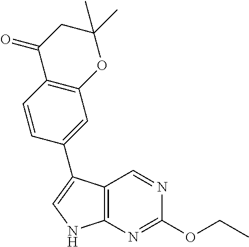 [SMILES: CCOc1ncc2c(-c3ccc4c(c3)OC(C)(C)CC4=O)c[nH]c2n1] | 1789 |
| 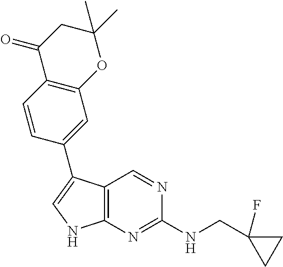 [SMILES: CC1(C)CC(=O)c2ccc(-c3c[nH]c4nc(NCC5(F)CC5)ncc34)cc2O1] | 1790 |
| 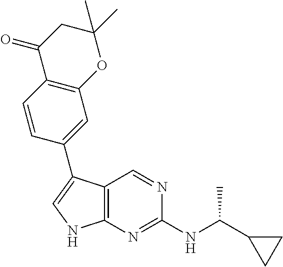 [SMILES: C[C@@H](Nc1ncc2c(-c3ccc4c(c3)OC(C)(C)CC4=O)c[nH]c2n1)C1CC1] | 1791 |
| 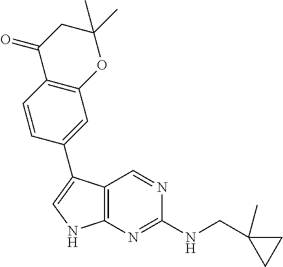 [SMILES: CC1(CNc2ncc3c(-c4ccc5c(c4)OC(C)(C)CC5=O)c[nH]c3n2)CC1] | 1792 |
| 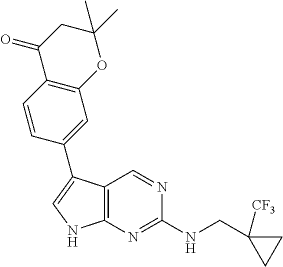 [SMILES: CC1(C)CC(=O)c2ccc(-c3c[nH]c4nc(NCC5(C(F)(F)F)CC5)ncc34)cc2O1] | 1793 |
| 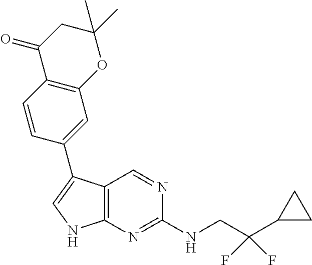 [SMILES: CC1(C)CC(=O)c2ccc(-c3c[nH]c4nc(NCC(F)(F)C5CC5)ncc34)cc2O1] | 1794 |
| 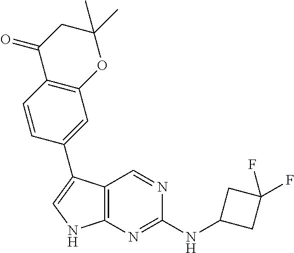 [SMILES: CC1(C)CC(=O)c2ccc(-c3c[nH]c4nc(NC5CC(F)(F)C5)ncc34)cc2O1] | 1795 |
| 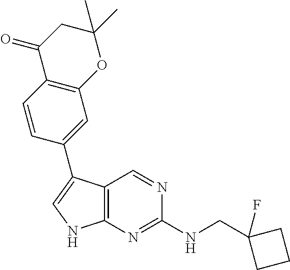 [SMILES: CC1(C)CC(=O)c2ccc(-c3c[nH]c4nc(NCC5(F)CCC5)ncc34)cc2O1] | 1796 |
| 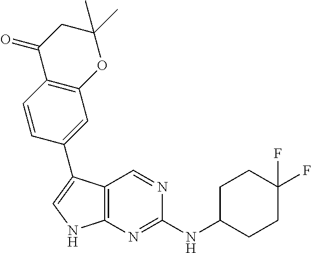 [SMILES: CC1(C)CC(=O)c2ccc(-c3c[nH]c4nc(NC5CCC(F)(F)CC5)ncc34)cc2O1] | 1797 |
| 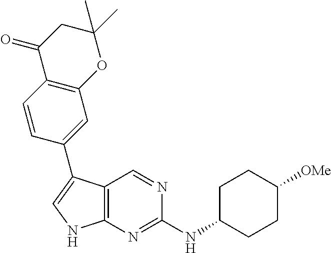 [SMILES: CO[C@H]1CC[C@@H](Nc2ncc3c(-c4ccc5c(c4)OC(C)(C)CC5=O)c[nH]c3n2)CC1] | 1798 |
| 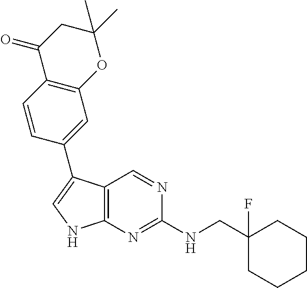 [SMILES: CC1(C)CC(=O)c2ccc(-c3c[nH]c4nc(NCC5(F)CCCCC5)ncc34)cc2O1] | 1799 |
| 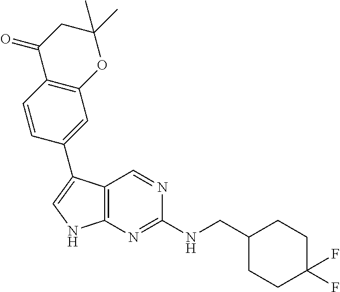 [SMILES: CC1(C)CC(=O)c2ccc(-c3c[nH]c4nc(NCC5CCC(F)(F)CC5)ncc34)cc2O1] | 1800 |
| 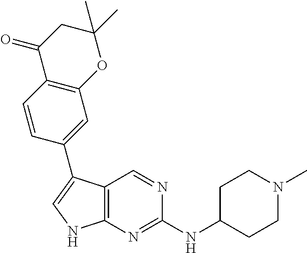 [SMILES: CN1CCC(Nc2ncc3c(-c4ccc5c(c4)OC(C)(C)CC5=O)c[nH]c3n2)CC1] | 1801 |
| 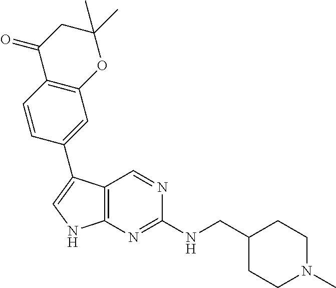 [SMILES: CN1CCC(CNc2ncc3c(-c4ccc5c(c4)OC(C)(C)CC5=O)c[nH]c3n2)CC1] | 1802 |
| 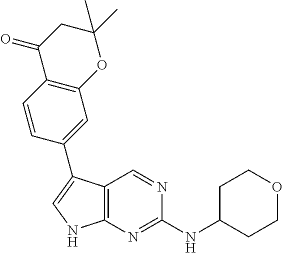 [SMILES: CC1(C)CC(=O)c2ccc(-c3c[nH]c4nc(NC5CCOCC5)ncc34)cc2O1] | 1803 |
| 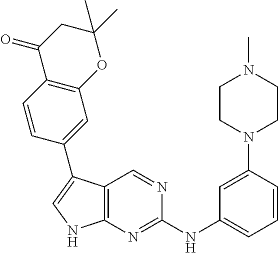 [SMILES: CN1CCN(c2cccc(Nc3ncc4c(-c5ccc6c(c5)OC(C)(C)CC6=O)c[nH]c4n3)c2)CC1] | 1804 |
| 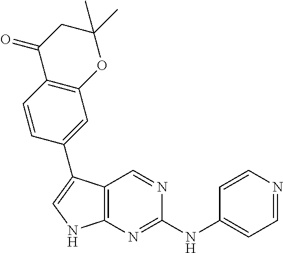 [SMILES: CC1(C)CC(=O)c2ccc(-c3c[nH]c4nc(Nc5ccncc5)ncc34)cc2O1] | 1805 |
| 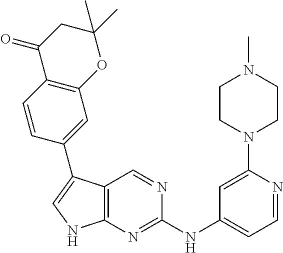 [SMILES: CN1CCN(c2cc(Nc3ncc4c(-c5ccc6c(c5)OC(C)(C)CC6=O)c[nH]c4n3)ccn2)CC1] | 1806 |
| 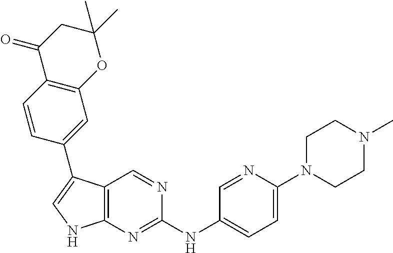 [SMILES: CN1CCN(c2ccc(Nc3ncc4c(-c5ccc6c(c5)OC(C)(C)CC6=O)c[nH]c4n3)cn2)CC1] | 1807 |
| 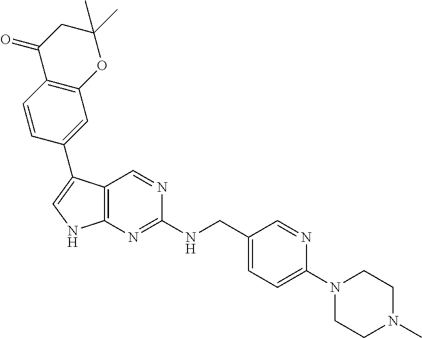 [SMILES: CN1CCN(c2ccc(CNc3ncc4c(-c5ccc6c(c5)OC(C)(C)CC6=O)c[nH]c4n3)cn2)CC1] | 1808 |
| 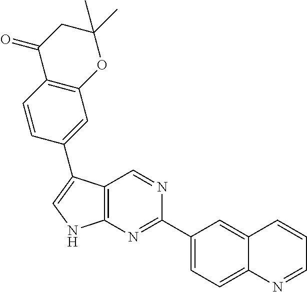 [SMILES: CC1(C)CC(=O)c2ccc(-c3c[nH]c4nc(-c5ccc6ncccc6c5)ncc34)cc2O1] | 1809 |
| 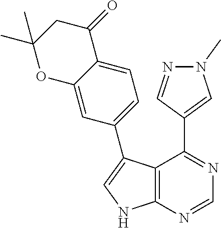 | 1810 |
| 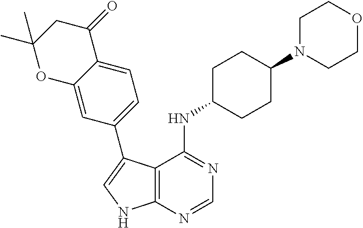 [SMILES: CC1(C)CC(=O)c2ccc(-c3c[nH]c4ncnc(N[C@H]5CC[C@H](N6CCOCC6)CC5)c34)cc2O1] | 1811 |
| 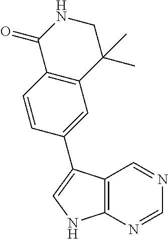 [SMILES: CC1(C)CNC(=O)c2ccc(-c3c[nH]c4ncncc34)cc21] | 1812 |
| 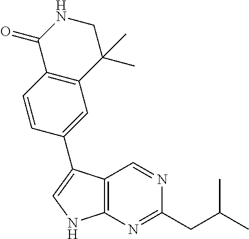 [SMILES: CC(C)Cc1ncc2c(-c3ccc4c(c3)C(C)(C)CNC4=O)c[nH]c2n1] | 1813 |
| 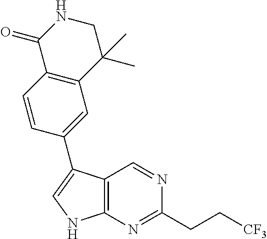 [SMILES: CC1(C)CNC(=O)c2ccc(-c3c[nH]c4nc(CCC(F)(F)F)ncc34)cc21] | 1814 |
| 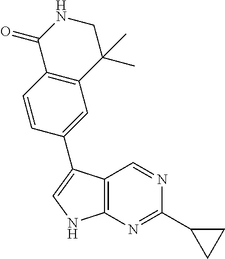 [SMILES: CC1(C)CNC(=O)c2ccc(-c3c[nH]c4nc(C5CC5)ncc34)cc21] | 1815 |
| 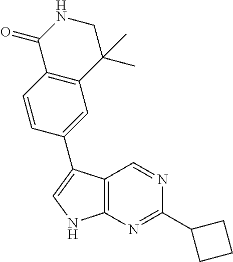 [SMILES: CC1(C)CNC(=O)c2ccc(-c3c[nH]c4nc(C5CCC5)ncc34)cc21] | 1816 |
| 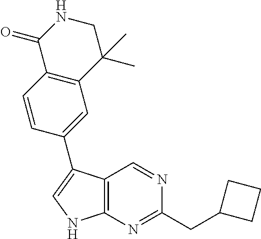 [SMILES: CC1(C)CNC(=O)c2ccc(-c3c[nH]c4nc(CC5CCC5)ncc34)cc21] | 1817 |
| 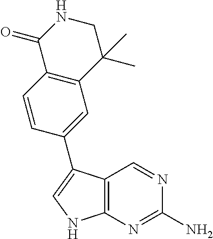 [SMILES: CC1(C)CNC(=O)c2ccc(-c3c[nH]c4nc(N)ncc34)cc21] | 1818 |
| 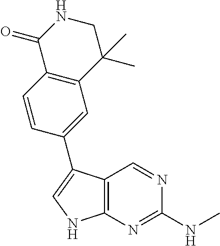 [SMILES: CNc1ncc2c(-c3ccc4c(c3)C(C)(C)CNC4=O)c[nH]c2n1] | 1819 |
| 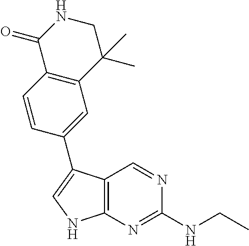 [SMILES: CCNc1ncc2c(-c3ccc4c(c3)C(C)(C)CNC4=O)c[nH]c2n1] | 1820 |
| 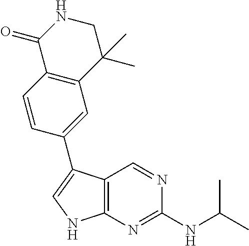 [SMILES: CC(C)Nc1ncc2c(-c3ccc4c(c3)C(C)(C)CNC4=O)c[nH]c2n1] | 1821 |
| 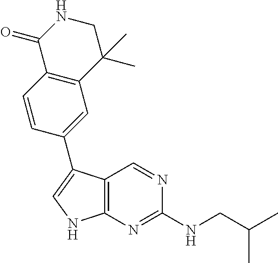 [SMILES: CC(C)CNc1ncc2c(-c3ccc4c(c3)C(C)(C)CNC4=O)c[nH]c2n1] | 1822 |
| 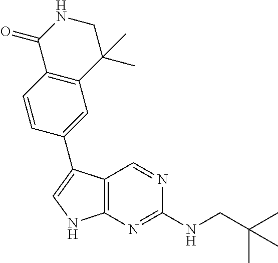 [SMILES: CC(C)(C)CNc1ncc2c(-c3ccc4c(c3)C(C)(C)CNC4=O)c[nH]c2n1] | 1823 |
| 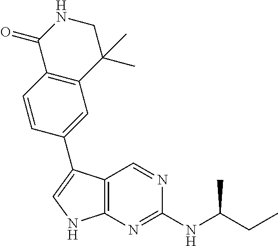 [SMILES: CC[C@H](C)Nc1ncc2c(-c3ccc4c(c3)C(C)(C)CNC4=O)c[nH]c2n1] | 1824 |
| 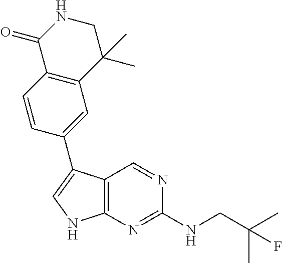 [SMILES: CC(C)(F)CNc1ncc2c(-c3ccc4c(c3)C(C)(C)CNC4=O)c[nH]c2n1] | 1825 |
| 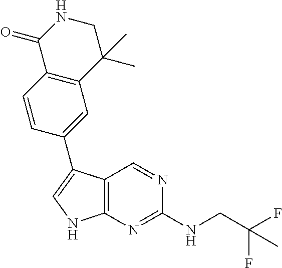 [SMILES: CC(F)(F)CNc1ncc2c(-c3ccc4c(c3)C(C)(C)CNC4=O)c[nH]c2n1] | 1826 |
| 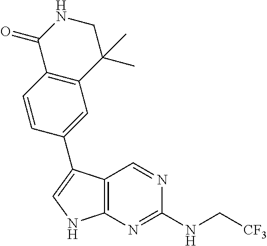 [SMILES: CC1(C)CNC(=O)c2ccc(-c3c[nH]c4nc(NCC(F)(F)F)ncc34)cc21] | 1827 |
| 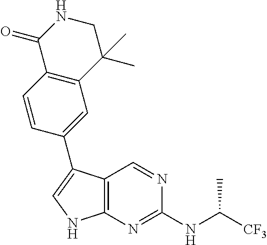 [SMILES: C[C@@H](Nc1ncc2c(-c3ccc4c(c3)C(C)(C)CNC4=O)c[nH]c2n1)C(F)(F)F] | 1828 |
| 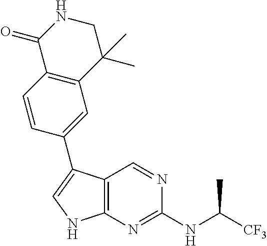 [SMILES: C[C@H](Nc1ncc2c(-c3ccc4c(c3)C(C)(C)CNC4=O)c[nH]c2n1)C(F)(F)F] | 1829 |
| 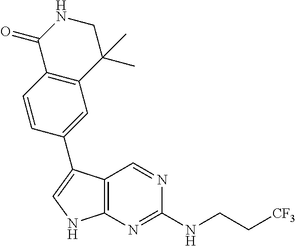 [SMILES: CC1(C)CNC(=O)c2ccc(-c3c[nH]c4nc(NCCC(F)(F)F)ncc34)cc21] | 1830 |
| 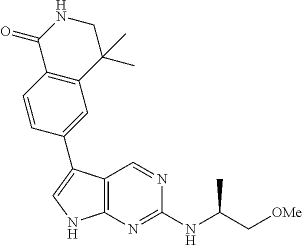 [SMILES: COC[C@H](C)Nc1ncc2c(-c3ccc4c(c3)C(C)(C)CNC4=O)c[nH]c2n1] | 1831 |
| 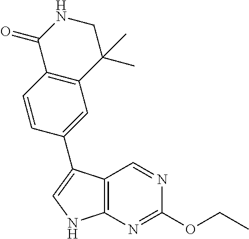 [SMILES: CCOc1ncc2c(-c3ccc4c(c3)C(C)(C)CNC4=O)c[nH]c2n1] | 1832 |
| 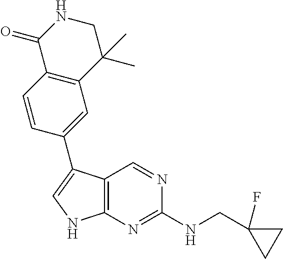 [SMILES: CC1(C)CNC(=O)c2ccc(-c3c[nH]c4nc(NCC5(F)CC5)ncc34)cc21] | 1833 |
| 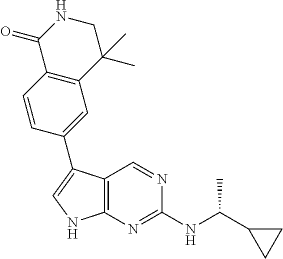 [SMILES: C[C@@H](Nc1ncc2c(-c3ccc4c(c3)C(C)(C)CNC4=O)c[nH]c2n1)C1CC1] | 1834 |
| 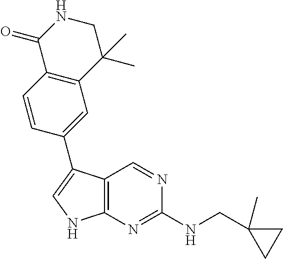 [SMILES: CC1(CNc2ncc3c(-c4ccc5c(c4)C(C)(C)CNC5=O)c[nH]c3n2)CC1] | 1835 |
| 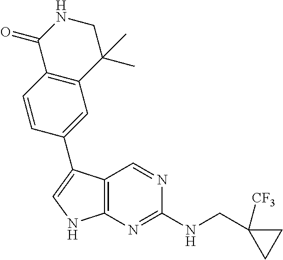 [SMILES: CC1(C)CNC(=O)c2ccc(-c3c[nH]c4nc(NCC5(C(F)(F)F)CC5)ncc34)cc21] | 1836 |
| 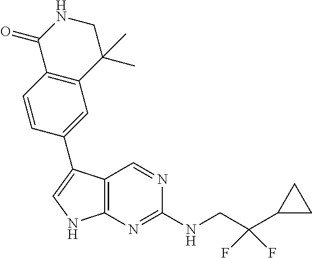 [SMILES: CC1(C)CNC(=O)c2ccc(-c3c[nH]c4nc(NCC(F)(F)C5CC5)ncc34)cc21] | 1837 |
| 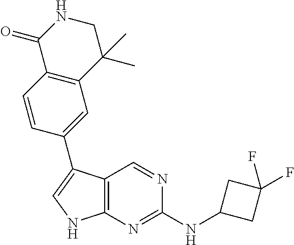 [SMILES: CC1(C)CNC(=O)c2ccc(-c3c[nH]c4nc(NC5CC(F)(F)C5)ncc34)cc21] | 1838 |
| 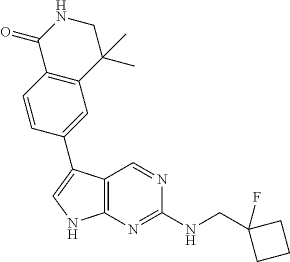 [SMILES: CC1(C)CNC(=O)c2ccc(-c3c[nH]c4nc(NCC5(F)CCC5)ncc34)cc21] | 1839 |
| 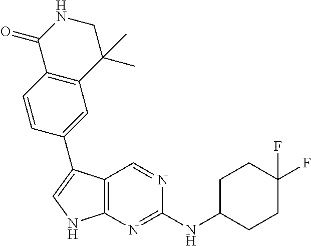 [SMILES: CC1(C)CNC(=O)c2ccc(-c3c[nH]c4nc(NC5CCC(F)(F)CC5)ncc34)cc21] | 1840 |
| 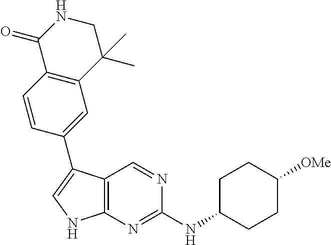 [SMILES: CO[C@H]1CC[C@@H](Nc2ncc3c(-c4ccc5c(c4)C(C)(C)CNC5=O)c[nH]c3n2)CC1] | 1841 |
| 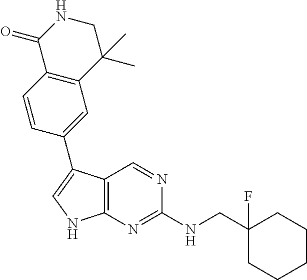 [SMILES: CC1(C)CNC(=O)c2ccc(-c3c[nH]c4nc(NCC5(F)CCCCC5)ncc34)cc21] | 1842 |
| 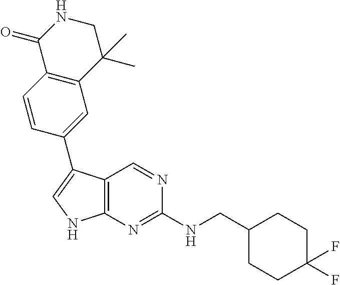 [SMILES: CC1(C)CNC(=O)c2ccc(-c3c[nH]c4nc(NCC5CCC(F)(F)CC5)ncc34)cc21] | 1843 |
| 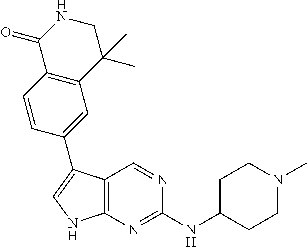 [SMILES: CN1CCC(Nc2ncc3c(-c4ccc5c(c4)C(C)(C)CNC5=O)c[nH]c3n2)CC1] | 1844 |
| 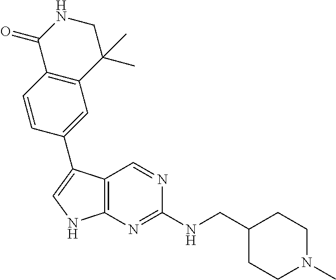 [SMILES: CN1CCC(CNc2ncc3c(-c4ccc5c(c4)C(C)(C)CNC5=O)c[nH]c3n2)CC1] | 1845 |
| 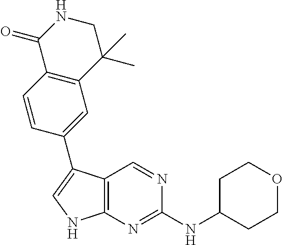 [SMILES: CC1(C)CNC(=O)c2ccc(-c3c[nH]c4nc(NC5CCOCC5)ncc34)cc21] | 1846 |
| 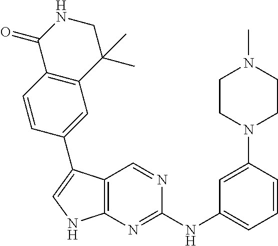 [SMILES: CN1CCN(c2cccc(Nc3ncc4c(-c5ccc6c(c5)C(C)(C)CNC6=O)c[nH]c4n3)c2)CC1] | 1847 |
| 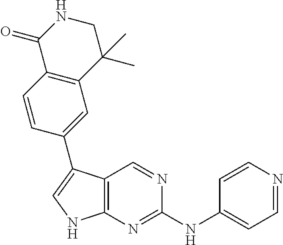 [SMILES: CC1(C)CNC(=O)c2ccc(-c3c[nH]c4nc(Nc5ccncc5)ncc34)cc21] | 1848 |
| 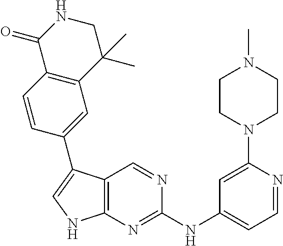 [SMILES: CN1CCN(c2cc(Nc3ncc4c(-c5ccc6c(c5)C(C)(C)CNC6=O)c[nH]c4n3)ccn2)CC1] | 1849 |
| 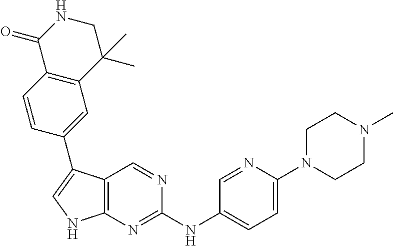 [SMILES: CN1CCN(c2ccc(Nc3ncc4c(-c5ccc6c(c5)C(C)(C)CNC6=O)c[nH]c4n3)cn2)CC1] | 1850 |
| 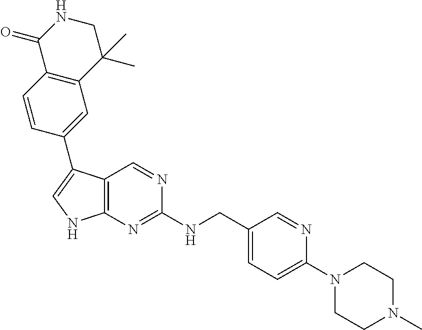 [SMILES: CN1CCN(c2ccc(CNc3ncc4c(-c5ccc6c(c5)C(C)(C)CNC6=O)c[nH]c4n3)cn2)CC1] | 1851 |
| 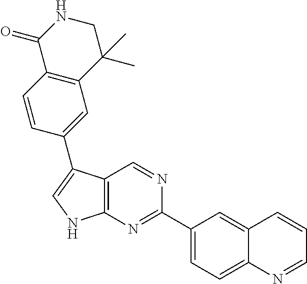 [SMILES: CC1(C)CNC(=O)c2ccc(-c3c[nH]c4nc(-c5ccc6ncccc6c5)ncc34)cc21] | 1852 |
| 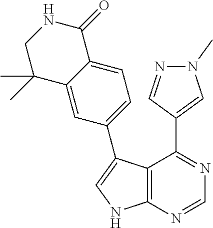 | 1853 |
| 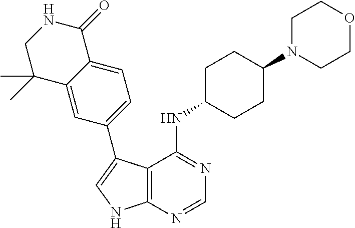 [SMILES: CC1(C)CNC(=O)c2ccc(-c3c[nH]c4ncnc(N[C@H]5CC[C@H](N6CCOCC6)CC5)c34)cc21] | 1854 |
| 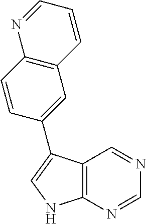 [SMILES: c1cnc2ccc(-c3c[nH]c4ncncc34)cc2c1] | 1855 |
| 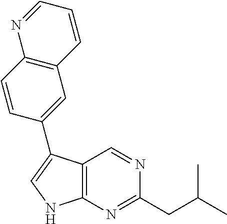 [SMILES: CC(C)Cc1ncc2c(-c3ccc4ncccc4c3)c[nH]c2n1] | 1856 |
| 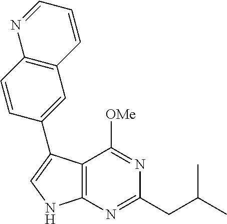 [SMILES: COc1nc(CC(C)C)nc2[nH]cc(-c3ccc4ncccc4c3)c12] | 1857 |
| 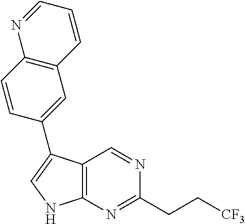 [SMILES: FC(F)(F)CCc1ncc2c(-c3ccc4ncccc4c3)c[nH]c2n1] | 1858 |
| 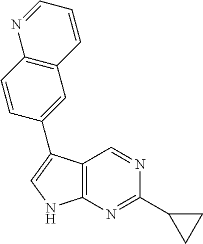 [SMILES: c1cnc2ccc(-c3c[nH]c4nc(C5CC5)ncc34)cc2c1] | 1859 |
| 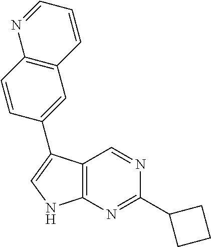 [SMILES: c1cnc2ccc(-c3c[nH]c4nc(C5CCC5)ncc34)cc2c1] | 1860 |
| 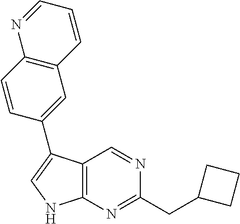 [SMILES: c1cnc2ccc(-c3c[nH]c4nc(CC5CCC5)ncc34)cc2c1] | 1861 |
| 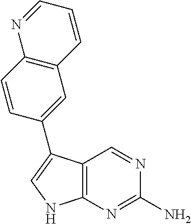 [SMILES: Nc1ncc2c(-c3ccc4ncccc4c3)c[nH]c2n1] | 1862 |
| 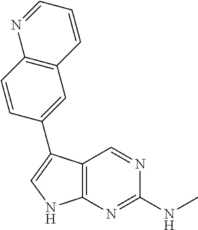 [SMILES: CNc1ncc2c(-c3ccc4ncccc4c3)c[nH]c2n1] | 1863 |
| 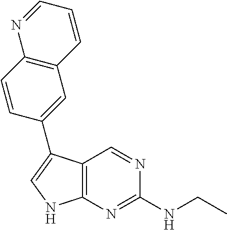 [SMILES: CCNc1ncc2c(-c3ccc4ncccc4c3)c[nH]c2n1] | 1864 |
| 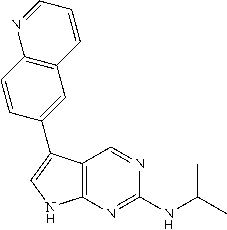 [SMILES: CC(C)Nc1ncc2c(-c3ccc4ncccc4c3)c[nH]c2n1] | 1865 |
| 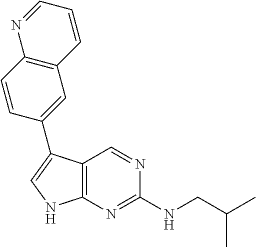 [SMILES: CC(C)CNc1ncc2c(-c3ccc4ncccc4c3)c[nH]c2n1] | 1866 |
| 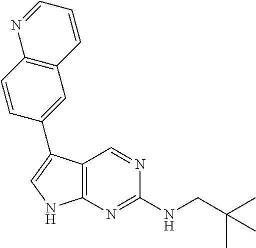 [SMILES: CC(C)(C)CNc1ncc2c(-c3ccc4ncccc4c3)c[nH]c2n1] | 1867 |
| 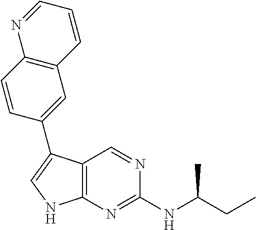 [SMILES: CC[C@H](C)Nc1ncc2c(-c3ccc4ncccc4c3)c[nH]c2n1] | 1868 |
| 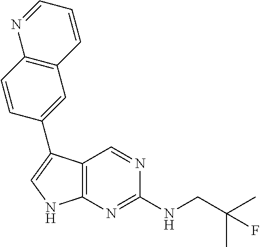 [SMILES: CC(C)(F)CNc1ncc2c(-c3ccc4ncccc4c3)c[nH]c2n1] | 1869 |
| 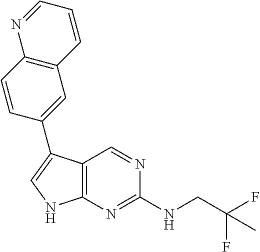 [SMILES: CC(F)(F)CNc1ncc2c(-c3ccc4ncccc4c3)c[nH]c2n1] | 1870 |
| 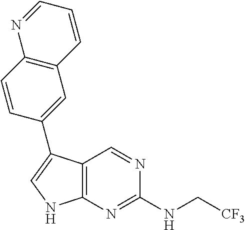 [SMILES: FC(F)(F)CNc1ncc2c(-c3ccc4ncccc4c3)c[nH]c2n1] | 1871 |
| 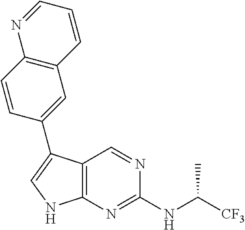 [SMILES: C[C@@H](Nc1ncc2c(-c3ccc4ncccc4c3)c[nH]c2n1)C(F)(F)F] | 1872 |
| 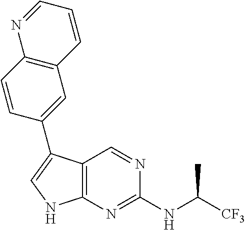 [SMILES: C[C@H](Nc1ncc2c(-c3ccc4ncccc4c3)c[nH]c2n1)C(F)(F)F] | 1873 |
| 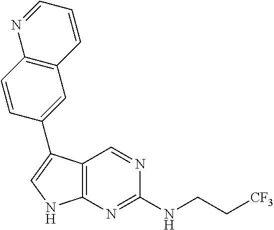 [SMILES: FC(F)(F)CCNc1ncc2c(-c3ccc4ncccc4c3)c[nH]c2n1] | 1874 |
| 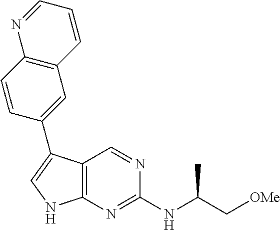 [SMILES: COC[C@H](C)Nc1ncc2c(-c3ccc4ncccc4c3)c[nH]c2n1] | 1875 |
| 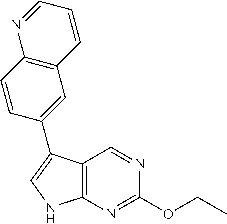 [SMILES: CCOc1ncc2c(-c3ccc4ncccc4c3)c[nH]c2n1] | 1876 |
| 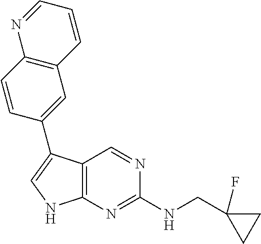 [SMILES: FC1(CNc2ncc3c(-c4ccc5ncccc5c4)c[nH]c3n2)CC1] | 1877 |
| 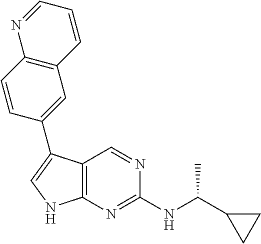 [SMILES: C[C@@H](Nc1ncc2c(-c3ccc4ncccc4c3)c[nH]c2n1)C1CC1] | 1878 |
| 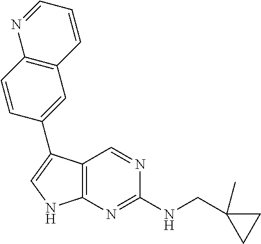 [SMILES: CC1(CNc2ncc3c(-c4ccc5ncccc5c4)c[nH]c3n2)CC1] | 1879 |
| 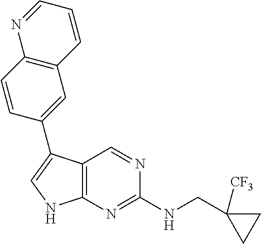 [SMILES: FC(F)(F)C1(CNc2ncc3c(-c4ccc5ncccc5c4)c[nH]c3n2)CC1] | 1880 |
| 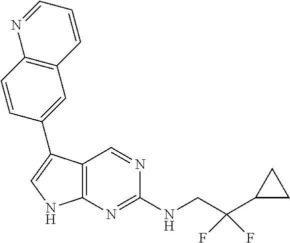 [SMILES: FC(F)(CNc1ncc2c(-c3ccc4ncccc4c3)c[nH]c2n1)C1CC1] | 1881 |
| 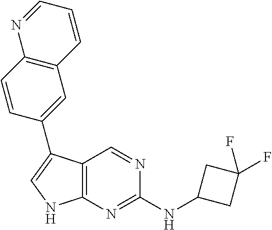 [SMILES: FC1(F)CC(Nc2ncc3c(-c4ccc5ncccc5c4)c[nH]c3n2)C1] | 1882 |
| 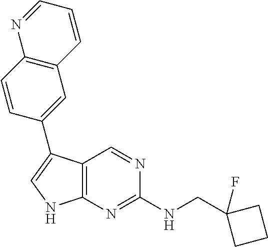 [SMILES: FC1(CNc2ncc3c(-c4ccc5ncccc5c4)c[nH]c3n2)CCC1] | 1883 |
| 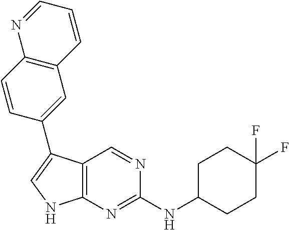 [SMILES: FC1(F)CCC(Nc2ncc3c(-c4ccc5ncccc5c4)c[nH]c3n2)CC1] | 1884 |
| 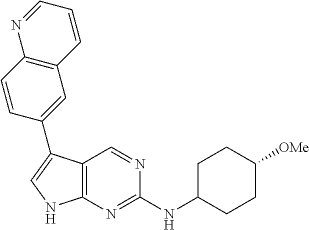 | 1885 |
| 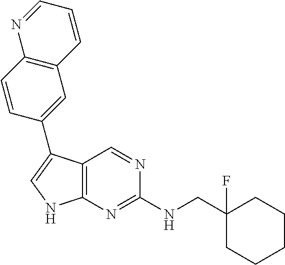 [SMILES: FC1(CNc2ncc3c(-c4ccc5ncccc5c4)c[nH]c3n2)CCCCC1] | 1886 |
| 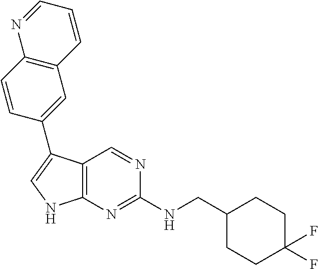 [SMILES: FC1(F)CCC(CNc2ncc3c(-c4ccc5ncccc5c4)c[nH]c3n2)CC1] | 1887 |
| 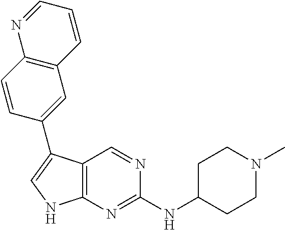 [SMILES: CN1CCC(Nc2ncc3c(-c4ccc5ncccc5c4)c[nH]c3n2)CC1] | 1888 |
| 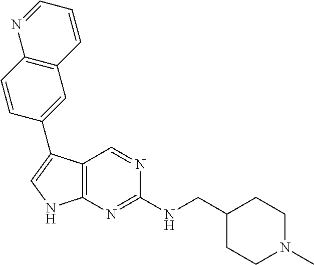 [SMILES: CN1CCC(CNc2ncc3c(-c4ccc5ncccc5c4)c[nH]c3n2)CC1] | 1889 |
| 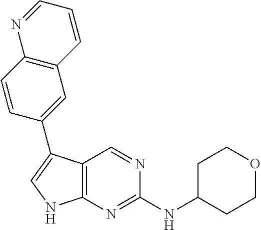 [SMILES: c1cnc2ccc(-c3c[nH]c4nc(NC5CCOCC5)ncc34)cc2c1] | 1890 |
| 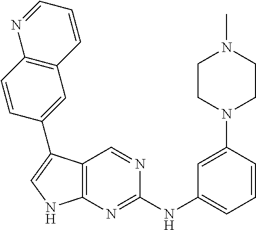 [SMILES: CN1CCN(c2cccc(Nc3ncc4c(-c5ccc6ncccc6c5)c[nH]c4n3)c2)CC1] | 1891 |
| 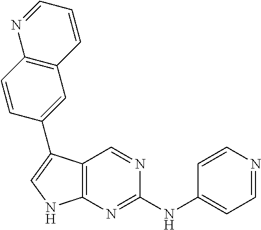 [SMILES: c1cnc2ccc(-c3c[nH]c4nc(Nc5ccncc5)ncc34)cc2c1] | 1892 |
| 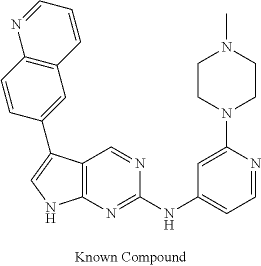 [SMILES: CN1CCN(c2cc(Nc3ncc4c(-c5ccc6ncccc6c5)c[nH]c4n3)ccn2)CC1] | 1893 |
| 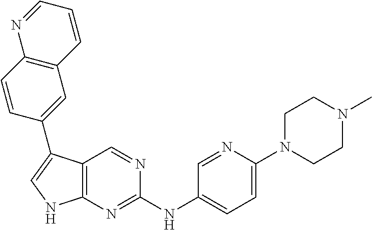 [SMILES: CN1CCN(c2ccc(Nc3ncc4c(-c5ccc6ncccc6c5)c[nH]c4n3)cn2)CC1] | 1894 |
| 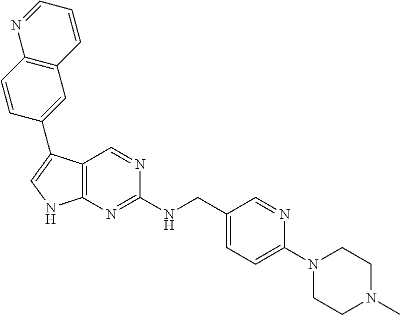 [SMILES: CN1CCN(c2ccc(CNc3ncc4c(-c5ccc6ncccc6c5)c[nH]c4n3)cn2)CC1] | 1895 |
| 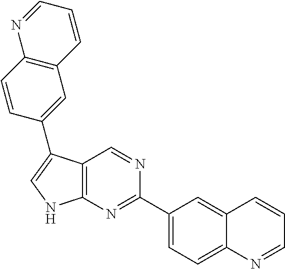 [SMILES: c1cnc2ccc(-c3ncc4c(-c5ccc6ncccc6c5)c[nH]c4n3)cc2c1] | 1896 |
| 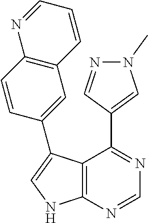 [SMILES: Cn1cc(-c2ncnc3[nH]cc(-c4ccc5ncccc5c4)c23)cn1] | 1897 |
| 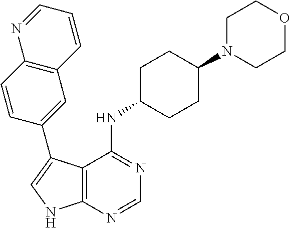 [SMILES: c1cnc2ccc(-c3c[nH]c4ncnc(N[C@H]5CC[C@H](N6CCOCC6)CC5)c34)cc2c1] | 1898 |
| 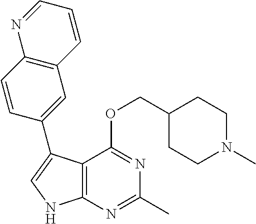 [SMILES: Cc1nc(OCC2CCN(C)CC2)c2c(-c3ccc4ncccc4c3)c[nH]c2n1] | 1899 |
| 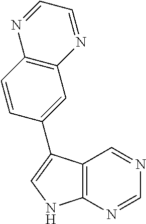 [SMILES: c1cnc2cc(-c3c[nH]c4ncncc34)ccc2n1] | 1900 |
| 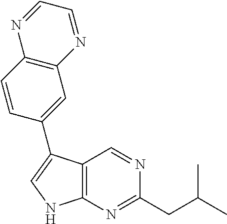 [SMILES: CC(C)Cc1ncc2c(-c3ccc4nccnc4c3)c[nH]c2n1] | 1901 |
| 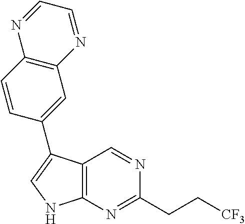 [SMILES: FC(F)(F)CCc1ncc2c(-c3ccc4nccnc4c3)c[nH]c2n1] | 1902 |
| 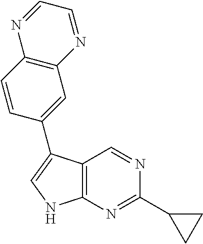 [SMILES: c1cnc2cc(-c3c[nH]c4nc(C5CC5)ncc34)ccc2n1] | 1903 |
| 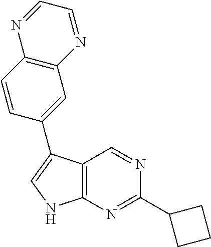 [SMILES: c1cnc2cc(-c3c[nH]c4nc(C5CCC5)ncc34)ccc2n1] | 1904 |
| 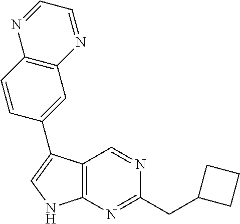 [SMILES: c1cnc2cc(-c3c[nH]c4nc(CC5CCC5)ncc34)ccc2n1] | 1905 |
| 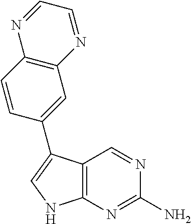 [SMILES: Nc1ncc2c(-c3ccc4nccnc4c3)c[nH]c2n1] | 1906 |
| 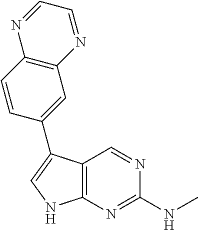 [SMILES: CNc1ncc2c(-c3ccc4nccnc4c3)c[nH]c2n1] | 1907 |
| 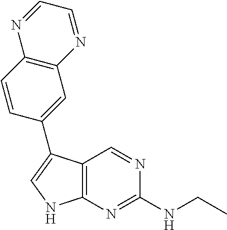 [SMILES: CCNc1ncc2c(-c3ccc4nccnc4c3)c[nH]c2n1] | 1908 |
| 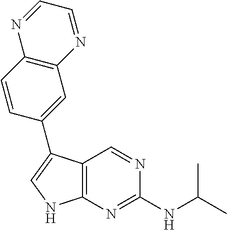 [SMILES: CC(C)Nc1ncc2c(-c3ccc4nccnc4c3)c[nH]c2n1] | 1909 |
| 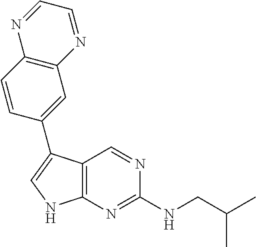 [SMILES: CC(C)CNc1ncc2c(-c3ccc4nccnc4c3)c[nH]c2n1] | 1910 |
| 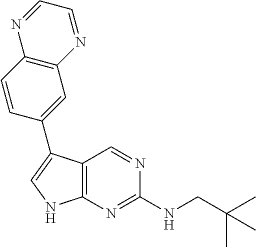 [SMILES: CC(C)(C)CNc1ncc2c(-c3ccc4nccnc4c3)c[nH]c2n1] | 1911 |
| 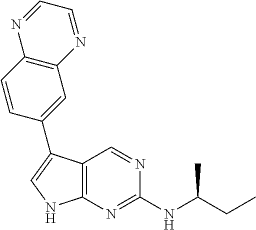 [SMILES: CC[C@H](C)Nc1ncc2c(-c3ccc4nccnc4c3)c[nH]c2n1] | 1912 |
| 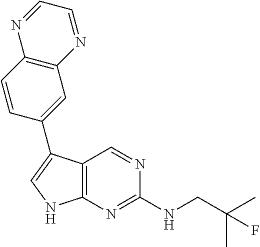 [SMILES: CC(C)(F)CNc1ncc2c(-c3ccc4nccnc4c3)c[nH]c2n1] | 1913 |
| 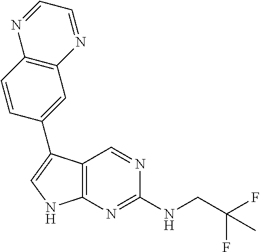 [SMILES: CC(F)(F)CNc1ncc2c(-c3ccc4nccnc4c3)c[nH]c2n1] | 1914 |
| 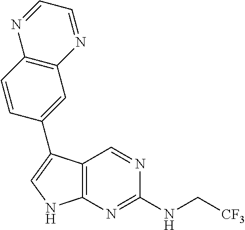 [SMILES: FC(F)(F)CNc1ncc2c(-c3ccc4nccnc4c3)c[nH]c2n1] | 1915 |
| 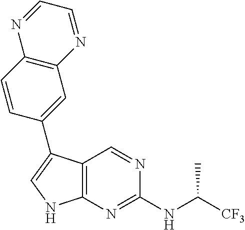 [SMILES: C[C@@H](Nc1ncc2c(-c3ccc4nccnc4c3)c[nH]c2n1)C(F)(F)F] | 1916 |
| 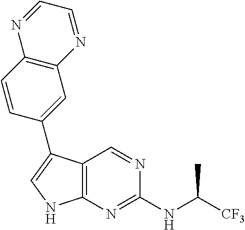 [SMILES: C[C@H](Nc1ncc2c(-c3ccc4nccnc4c3)c[nH]c2n1)C(F)(F)F] | 1917 |
| 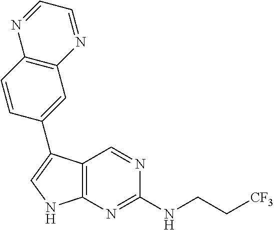 [SMILES: FC(F)(F)CCNc1ncc2c(-c3ccc4nccnc4c3)c[nH]c2n1] | 1918 |
| 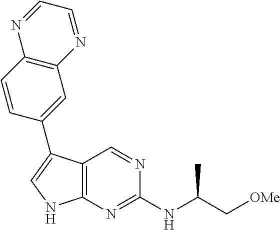 [SMILES: COC[C@H](C)Nc1ncc2c(-c3ccc4nccnc4c3)c[nH]c2n1] | 1919 |
| 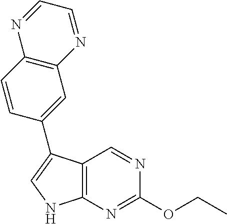 [SMILES: CCOc1ncc2c(-c3ccc4nccnc4c3)c[nH]c2n1] | 1920 |
| 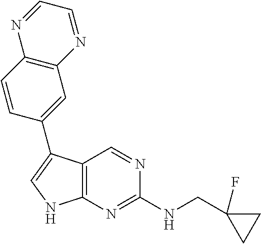 [SMILES: FC1(CNc2ncc3c(-c4ccc5nccnc5c4)c[nH]c3n2)CC1] | 1921 |
| 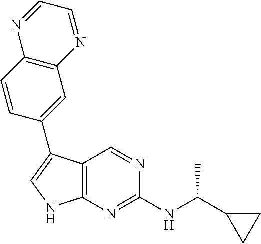 [SMILES: C[C@@H](Nc1ncc2c(-c3ccc4nccnc4c3)c[nH]c2n1)C1CC1] | 1922 |
| 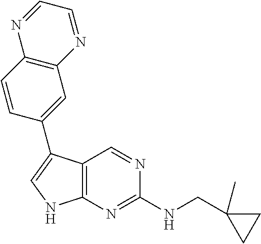 [SMILES: CC1(CNc2ncc3c(-c4ccc5nccnc5c4)c[nH]c3n2)CC1] | 1923 |
| 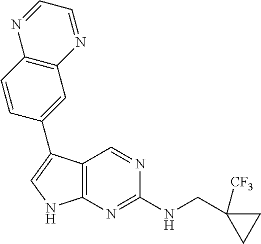 [SMILES: FC(F)(F)C1(CNc2ncc3c(-c4ccc5nccnc5c4)c[nH]c3n2)CC1] | 1924 |
| 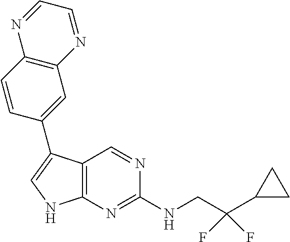 [SMILES: FC(F)(CNc1ncc2c(-c3ccc4nccnc4c3)c[nH]c2n1)C1CC1] | 1925 |
| 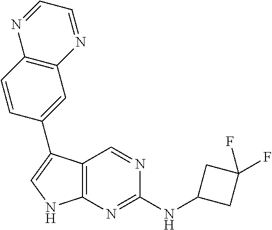 [SMILES: FC1(F)CC(Nc2ncc3c(-c4ccc5nccnc5c4)c[nH]c3n2)C1] | 1926 |
| 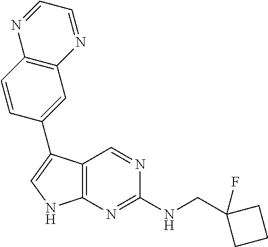 [SMILES: FC1(CNc2ncc3c(-c4ccc5nccnc5c4)c[nH]c3n2)CCC1] | 1927 |
| 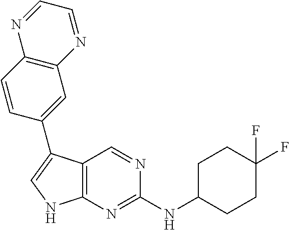 [SMILES: FC1(F)CCC(Nc2ncc3c(-c4ccc5nccnc5c4)c[nH]c3n2)CC1] | 1928 |
| 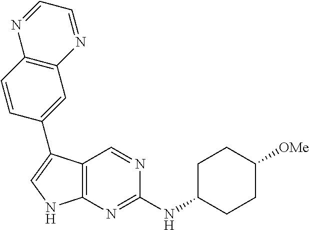 [SMILES: CO[C@H]1CC[C@@H](Nc2ncc3c(-c4ccc5nccnc5c4)c[nH]c3n2)CC1] | 1929 |
| 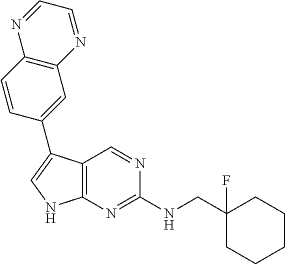 [SMILES: FC1(CNc2ncc3c(-c4ccc5nccnc5c4)c[nH]c3n2)CCCCC1] | 1930 |
| 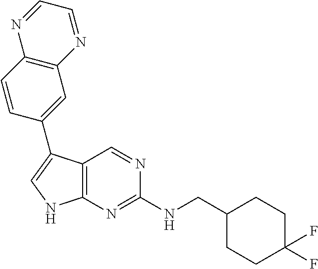 [SMILES: FC1(F)CCC(CNc2ncc3c(-c4ccc5nccnc5c4)c[nH]c3n2)CC1] | 1931 |
| 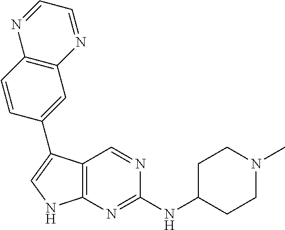 [SMILES: CN1CCC(Nc2ncc3c(-c4ccc5nccnc5c4)c[nH]c3n2)CC1] | 1932 |
| 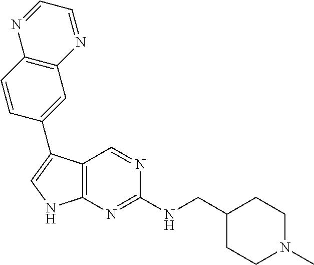 [SMILES: CN1CCC(CNc2ncc3c(-c4ccc5nccnc5c4)c[nH]c3n2)CC1] | 1933 |
| 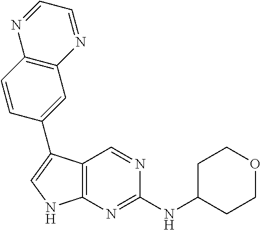 [SMILES: c1cnc2cc(-c3c[nH]c4nc(NC5CCOCC5)ncc34)ccc2n1] | 1934 |
| 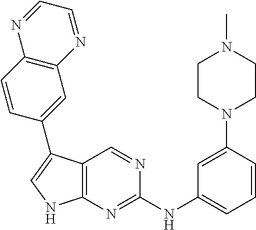 [SMILES: CN1CCN(c2cccc(Nc3ncc4c(-c5ccc6nccnc6c5)c[nH]c4n3)c2)CC1] | 1935 |
| 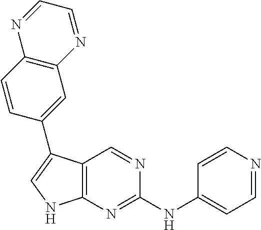 [SMILES: c1cc(Nc2ncc3c(-c4ccc5nccnc5c4)c[nH]c3n2)ccn1] | 1936 |
| 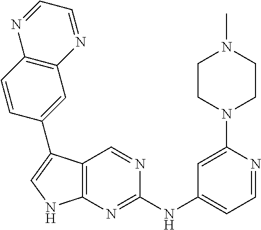 [SMILES: CN1CCN(c2cc(Nc3ncc4c(-c5ccc6nccnc6c5)c[nH]c4n3)ccn2)CC1] | 1937 |
| 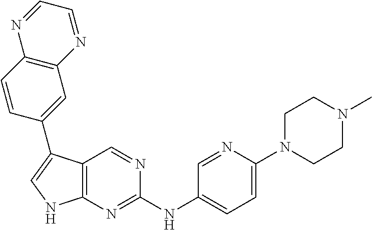 [SMILES: CN1CCN(c2ccc(Nc3ncc4c(-c5ccc6nccnc6c5)c[nH]c4n3)cn2)CC1] | 1938 |
| 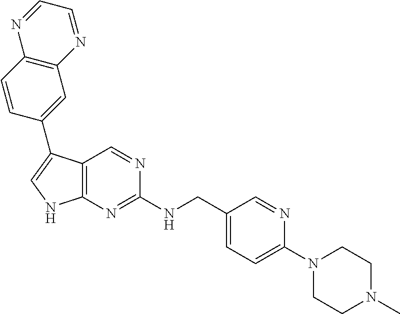 [SMILES: CN1CCN(c2ccc(CNc3ncc4c(-c5ccc6nccnc6c5)c[nH]c4n3)cn2)CC1] | 1939 |
| 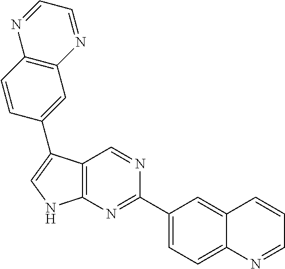 [SMILES: c1cnc2ccc(-c3ncc4c(-c5ccc6nccnc6c5)c[nH]c4n3)cc2c1] | 1940 |
| 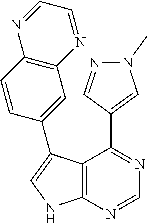 [SMILES: Cn1cc(-c2ncnc3[nH]cc(-c4ccc5nccnc5c4)c23)cn1] | 1941 |
| 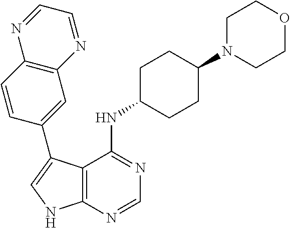 [SMILES: c1cnc2cc(-c3c[nH]c4ncnc(N[C@H]5CC[C@H](N6CCOCC6)CC5)c34)ccc2n1] | 1942 |
| 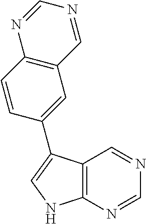 [SMILES: c1ncc2cc(-c3c[nH]c4ncncc34)ccc2n1] | 1943 |
| 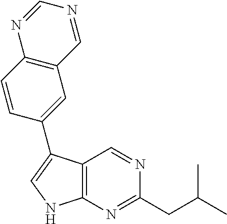 [SMILES: CC(C)Cc1ncc2c(-c3ccc4ncncc4c3)c[nH]c2n1] | 1944 |
| 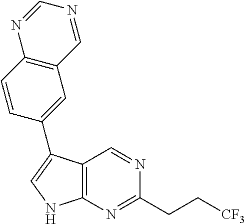 [SMILES: FC(F)(F)CCc1ncc2c(-c3ccc4ncncc4c3)c[nH]c2n1] | 1945 |
| 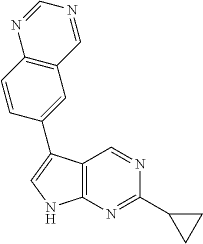 [SMILES: c1ncc2cc(-c3c[nH]c4nc(C5CC5)ncc34)ccc2n1] | 1946 |
| 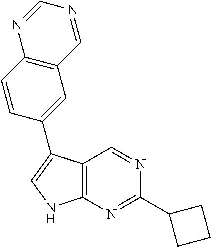 [SMILES: c1ncc2cc(-c3c[nH]c4nc(C5CCC5)ncc34)ccc2n1] | 1947 |
| 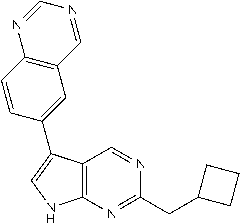 [SMILES: c1ncc2cc(-c3c[nH]c4nc(CC5CCC5)ncc34)ccc2n1] | 1948 |
| 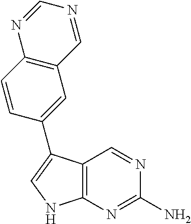 [SMILES: Nc1ncc2c(-c3ccc4ncncc4c3)c[nH]c2n1] | 1949 |
| 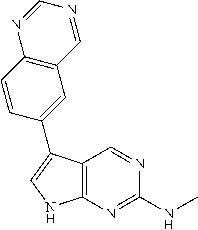 [SMILES: CNc1ncc2c(-c3ccc4ncncc4c3)c[nH]c2n1] | 1950 |
| 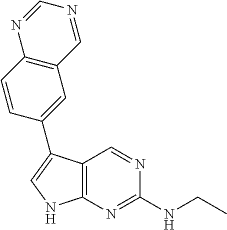 [SMILES: CCNc1ncc2c(-c3ccc4ncncc4c3)c[nH]c2n1] | 1951 |
| 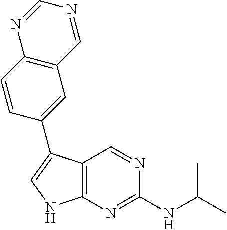 [SMILES: CC(C)Nc1ncc2c(-c3ccc4ncncc4c3)c[nH]c2n1] | 1952 |
| 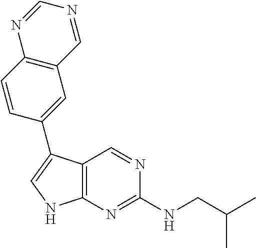 [SMILES: CC(C)CNc1ncc2c(-c3ccc4ncncc4c3)c[nH]c2n1] | 1953 |
| 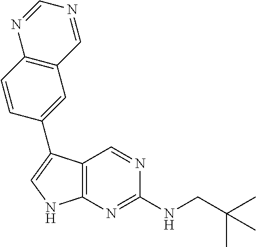 [SMILES: CC(C)(C)CNc1ncc2c(-c3ccc4ncncc4c3)c[nH]c2n1] | 1954 |
| 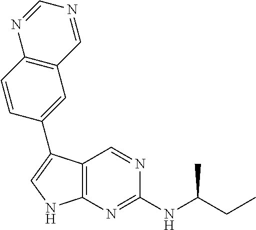 [SMILES: CC[C@H](C)Nc1ncc2c(-c3ccc4ncncc4c3)c[nH]c2n1] | 1955 |
| 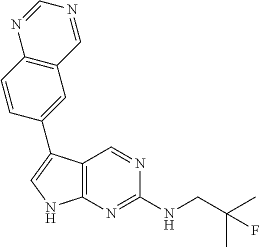 [SMILES: CC(C)(F)CNc1ncc2c(-c3ccc4ncncc4c3)c[nH]c2n1] | 1956 |
| 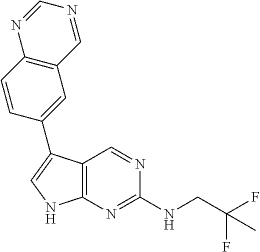 [SMILES: CC(F)(F)CNc1ncc2c(-c3ccc4ncncc4c3)c[nH]c2n1] | 1957 |
| 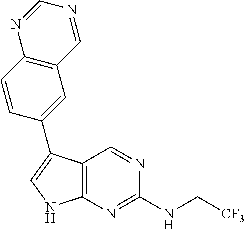 [SMILES: FC(F)(F)CNc1ncc2c(-c3ccc4ncncc4c3)c[nH]c2n1] | 1958 |
| 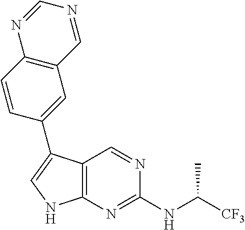 [SMILES: C[C@@H](Nc1ncc2c(-c3ccc4ncncc4c3)c[nH]c2n1)C(F)(F)F] | 1959 |
| 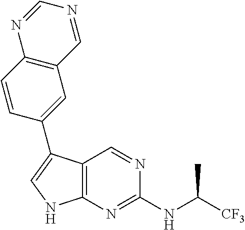 [SMILES: C[C@H](Nc1ncc2c(-c3ccc4ncncc4c3)c[nH]c2n1)C(F)(F)F] | 1960 |
| 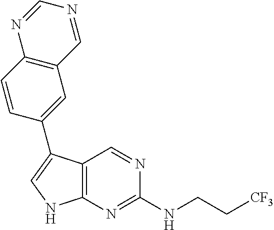 [SMILES: FC(F)(F)CCNc1ncc2c(-c3ccc4ncncc4c3)c[nH]c2n1] | 1961 |
| 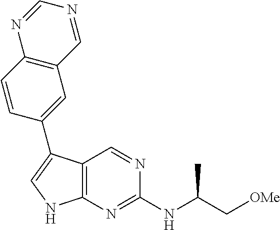 [SMILES: COC[C@H](C)Nc1ncc2c(-c3ccc4ncncc4c3)c[nH]c2n1] | 1962 |
| 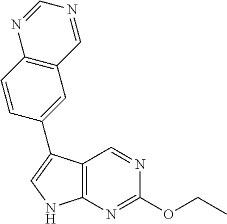 [SMILES: CCOc1ncc2c(-c3ccc4ncncc4c3)c[nH]c2n1] | 1963 |
| 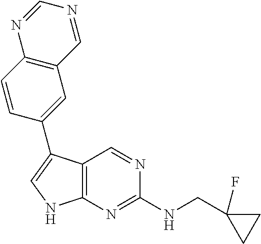 [SMILES: FC1(CNc2ncc3c(-c4ccc5ncncc5c4)c[nH]c3n2)CC1] | 1964 |
| 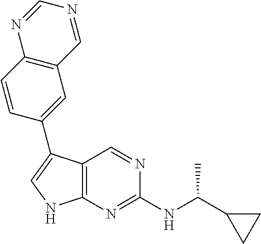 [SMILES: C[C@@H](Nc1ncc2c(-c3ccc4ncncc4c3)c[nH]c2n1)C1CC1] | 1965 |
| 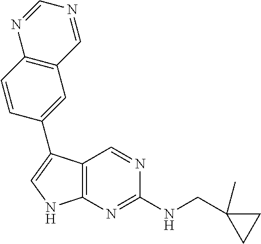 [SMILES: CC1(CNc2ncc3c(-c4ccc5ncncc5c4)c[nH]c3n2)CC1] | 1966 |
| 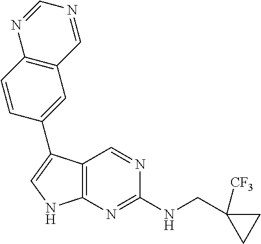 [SMILES: FC(F)(F)C1(CNc2ncc3c(-c4ccc5ncncc5c4)c[nH]c3n2)CC1] | 1967 |
| 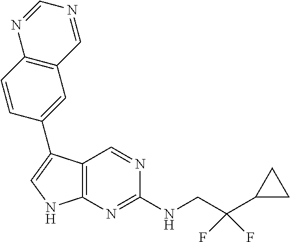 [SMILES: FC(F)(CNc1ncc2c(-c3ccc4ncncc4c3)c[nH]c2n1)C1CC1] | 1968 |
| 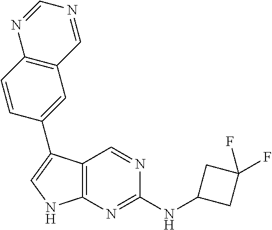 [SMILES: FC1(F)CC(Nc2ncc3c(-c4ccc5ncncc5c4)c[nH]c3n2)C1] | 1969 |
| 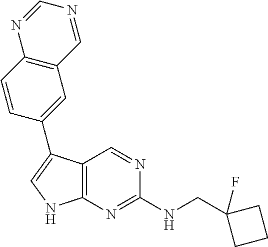 [SMILES: FC1(CNc2ncc3c(-c4ccc5ncncc5c4)c[nH]c3n2)CCC1] | 1970 |
| 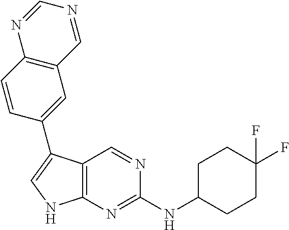 [SMILES: FC1(F)CCC(Nc2ncc3c(-c4ccc5ncncc5c4)c[nH]c3n2)CC1] | 1971 |
| 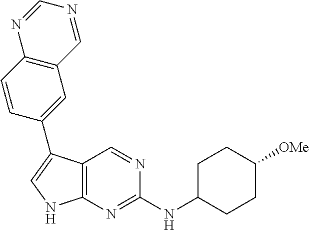 | 1972 |
| 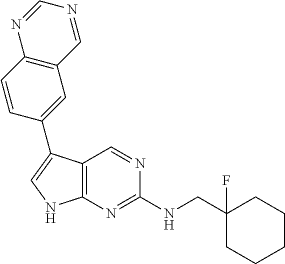 [SMILES: FC1(CNc2ncc3c(-c4ccc5ncncc5c4)c[nH]c3n2)CCCCC1] | 1973 |
| 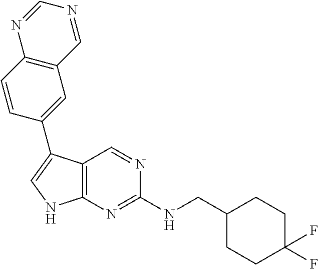 [SMILES: FC1(F)CCC(CNc2ncc3c(-c4ccc5ncncc5c4)c[nH]c3n2)CC1] | 1974 |
| 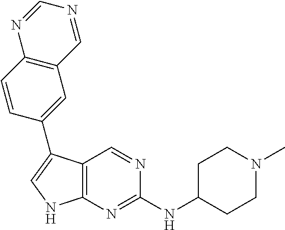 [SMILES: CN1CCC(Nc2ncc3c(-c4ccc5ncncc5c4)c[nH]c3n2)CC1] | 1975 |
| 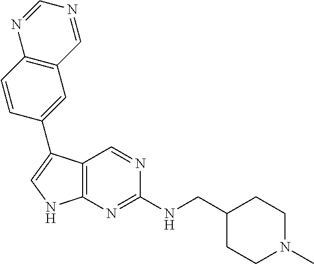 [SMILES: CN1CCC(CNc2ncc3c(-c4ccc5ncncc5c4)c[nH]c3n2)CC1] | 1976 |
| 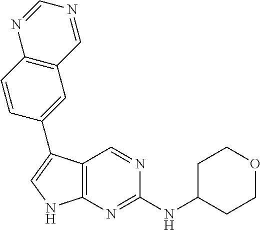 [SMILES: c1ncc2cc(-c3c[nH]c4nc(NC5CCOCC5)ncc34)ccc2n1] | 1977 |
| 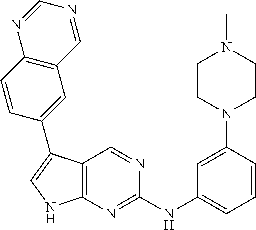 [SMILES: CN1CCN(c2cccc(Nc3ncc4c(-c5ccc6ncncc6c5)c[nH]c4n3)c2)CC1] | 1978 |
| 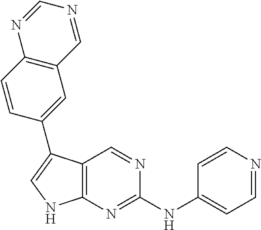 [SMILES: c1cc(Nc2ncc3c(-c4ccc5ncncc5c4)c[nH]c3n2)ccn1] | 1979 |
| 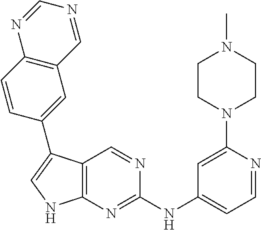 [SMILES: CN1CCN(c2cc(Nc3ncc4c(-c5ccc6ncncc6c5)c[nH]c4n3)ccn2)CC1] | 1980 |
| 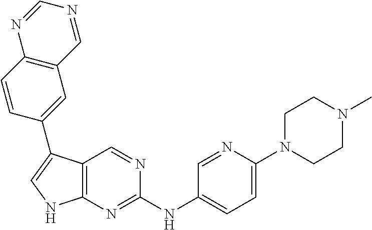 [SMILES: CN1CCN(c2ccc(Nc3ncc4c(-c5ccc6ncncc6c5)c[nH]c4n3)cn2)CC1] | 1981 |
| 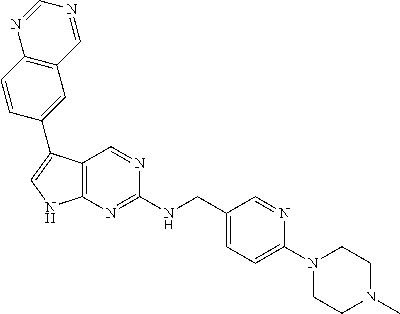 [SMILES: CN1CCN(c2ccc(CNc3ncc4c(-c5ccc6ncncc6c5)c[nH]c4n3)cn2)CC1] | 1982 |
| 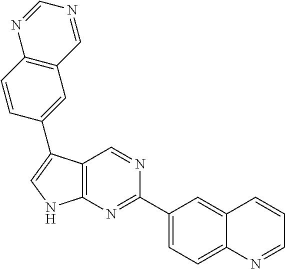 [SMILES: c1cnc2ccc(-c3ncc4c(-c5ccc6ncncc6c5)c[nH]c4n3)cc2c1] | 1983 |
| 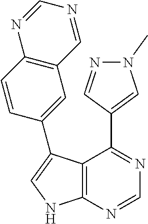 [SMILES: Cn1cc(-c2ncnc3[nH]cc(-c4ccc5ncncc5c4)c23)cn1] | 1984 |
| 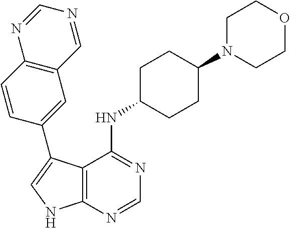 [SMILES: c1ncc2cc(-c3c[nH]c4ncnc(N[C@H]5CC[C@H](N6CCOCC6)CC5)c34)ccc2n1] | 1985 |
| 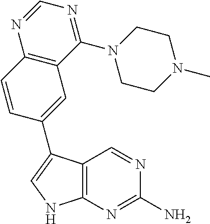 [SMILES: CN1CCN(c2ncnc3ccc(-c4c[nH]c5nc(N)ncc45)cc23)CC1] | 1986 |
| 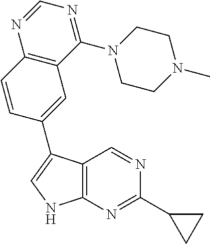 [SMILES: CN1CCN(c2ncnc3ccc(-c4c[nH]c5nc(C6CC6)ncc45)cc23)CC1] | 1987 |
| 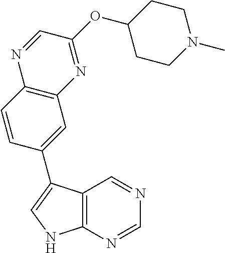 [SMILES: CN1CCC(Oc2cnc3ccc(-c4c[nH]c5ncncc45)cc3n2)CC1] | 1988 |
| 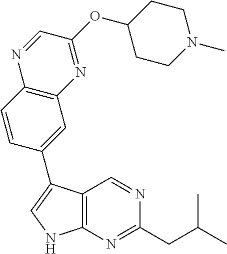 [SMILES: CC(C)Cc1ncc2c(-c3ccc4ncc(OC5CCN(C)CC5)nc4c3)c[nH]c2n1] | 1989 |
| 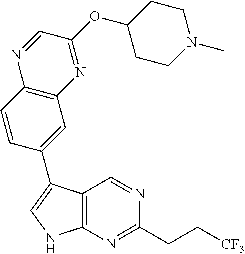 [SMILES: CN1CCC(Oc2cnc3ccc(-c4c[nH]c5nc(CCC(F)(F)F)ncc45)cc3n2)CC1] | 1990 |
| 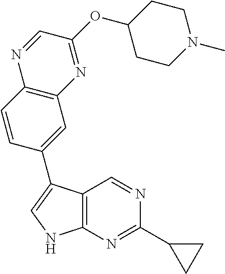 [SMILES: CN1CCC(Oc2cnc3ccc(-c4c[nH]c5nc(C6CC6)ncc45)cc3n2)CC1] | 1991 |
| 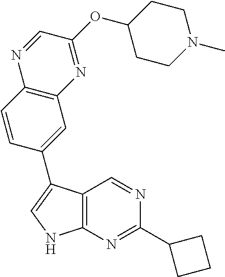 [SMILES: CN1CCC(Oc2cnc3ccc(-c4c[nH]c5nc(C6CCC6)ncc45)cc3n2)CC1] | 1992 |
| 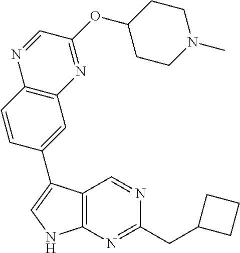 [SMILES: CN1CCC(Oc2cnc3ccc(-c4c[nH]c5nc(CC6CCC6)ncc45)cc3n2)CC1] | 1993 |
| 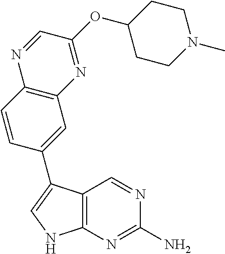 [SMILES: CN1CCC(Oc2cnc3ccc(-c4c[nH]c5nc(N)ncc45)cc3n2)CC1] | 1994 |
| 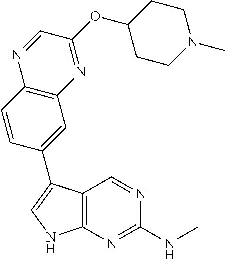 [SMILES: CNc1ncc2c(-c3ccc4ncc(OC5CCN(C)CC5)nc4c3)c[nH]c2n1] | 1995 |
| 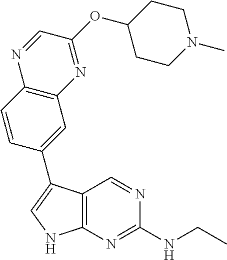 [SMILES: CCNc1ncc2c(-c3ccc4ncc(OC5CCN(C)CC5)nc4c3)c[nH]c2n1] | 1996 |
| 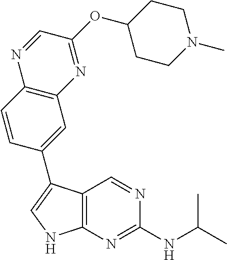 [SMILES: CC(C)Nc1ncc2c(-c3ccc4ncc(OC5CCN(C)CC5)nc4c3)c[nH]c2n1] | 1997 |
| 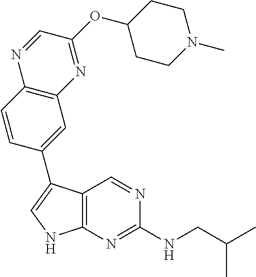 [SMILES: CC(C)CNc1ncc2c(-c3ccc4ncc(OC5CCN(C)CC5)nc4c3)c[nH]c2n1] | 1998 |
| 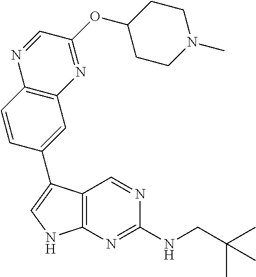 [SMILES: CN1CCC(Oc2cnc3ccc(-c4c[nH]c5nc(NCC(C)(C)C)ncc45)cc3n2)CC1] | 1999 |
| 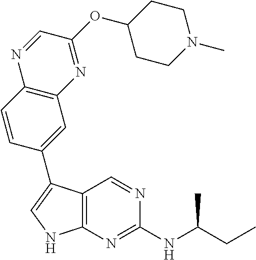 [SMILES: CC[C@H](C)Nc1ncc2c(-c3ccc4ncc(OC5CCN(C)CC5)nc4c3)c[nH]c2n1] | 2000 |
| 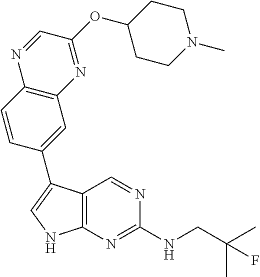 [SMILES: CN1CCC(Oc2cnc3ccc(-c4c[nH]c5nc(NCC(C)(C)F)ncc45)cc3n2)CC1] | 2001 |
| 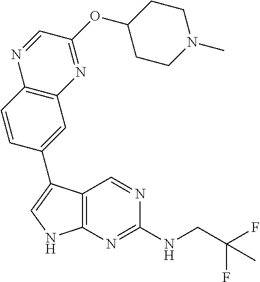 [SMILES: CN1CCC(Oc2cnc3ccc(-c4c[nH]c5nc(NCC(C)(F)F)ncc45)cc3n2)CC1] | 2002 |
| 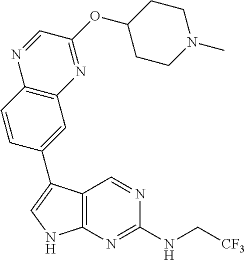 [SMILES: CN1CCC(Oc2cnc3ccc(-c4c[nH]c5nc(NCC(F)(F)F)ncc45)cc3n2)CC1] | 2003 |
| 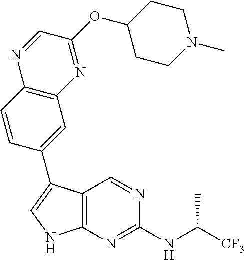 [SMILES: C[C@@H](Nc1ncc2c(-c3ccc4ncc(OC5CCN(C)CC5)nc4c3)c[nH]c2n1)C(F)(F)F] | 2004 |
| 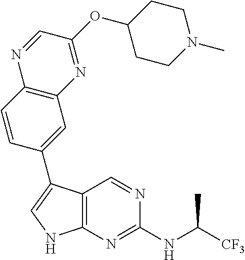 [SMILES: C[C@H](Nc1ncc2c(-c3ccc4ncc(OC5CCN(C)CC5)nc4c3)c[nH]c2n1)C(F)(F)F] | 2005 |
| 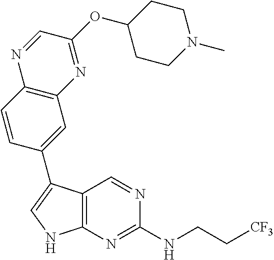 [SMILES: CN1CCC(Oc2cnc3ccc(-c4c[nH]c5nc(NCCC(F)(F)F)ncc45)cc3n2)CC1] | 2006 |
| 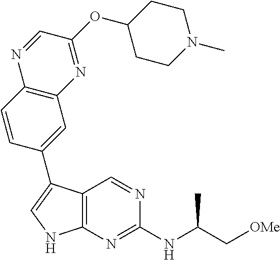 [SMILES: COC[C@H](C)Nc1ncc2c(-c3ccc4ncc(OC5CCN(C)CC5)nc4c3)c[nH]c2n1] | 2007 |
| 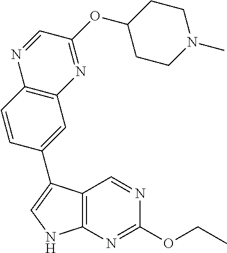 [SMILES: CCOc1ncc2c(-c3ccc4ncc(OC5CCN(C)CC5)nc4c3)c[nH]c2n1] | 2008 |
| 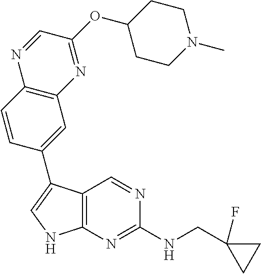 [SMILES: CN1CCC(Oc2cnc3ccc(-c4c[nH]c5nc(NCC6(F)CC6)ncc45)cc3n2)CC1] | 2009 |
| 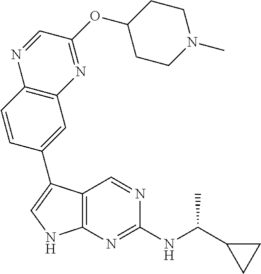 [SMILES: C[C@@H](Nc1ncc2c(-c3ccc4ncc(OC5CCN(C)CC5)nc4c3)c[nH]c2n1)C1CC1] | 2010 |
| 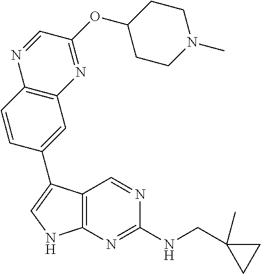 [SMILES: CN1CCC(Oc2cnc3ccc(-c4c[nH]c5nc(NCC6(C)CC6)ncc45)cc3n2)CC1] | 2011 |
| 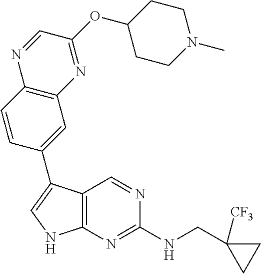 [SMILES: CN1CCC(Oc2cnc3ccc(-c4c[nH]c5nc(NCC6(C(F)(F)F)CC6)ncc45)cc3n2)CC1] | 2012 |
| 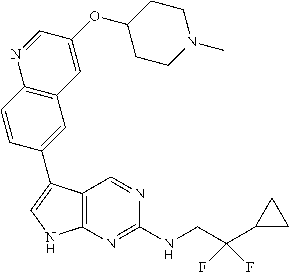 [SMILES: CN1CCC(Oc2cnc3ccc(-c4c[nH]c5nc(NCC(F)(F)C6CC6)ncc45)cc3c2)CC1] | 2013 |
| 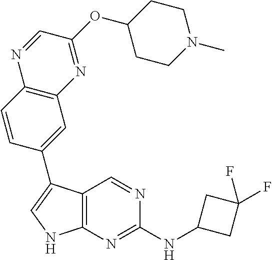 [SMILES: CN1CCC(Oc2cnc3ccc(-c4c[nH]c5nc(NC6CC(F)(F)C6)ncc45)cc3n2)CC1] | 2014 |
| 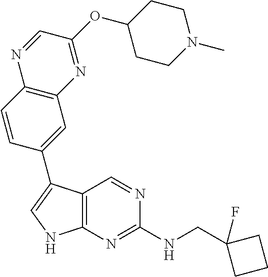 [SMILES: CN1CCC(Oc2cnc3ccc(-c4c[nH]c5nc(NCC6(F)CCC6)ncc45)cc3n2)CC1] | 2015 |
| 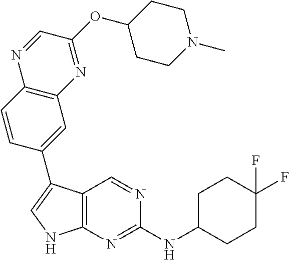 [SMILES: CN1CCC(Oc2cnc3ccc(-c4c[nH]c5nc(NC6CCC(F)(F)CC6)ncc45)cc3n2)CC1] | 2016 |
| 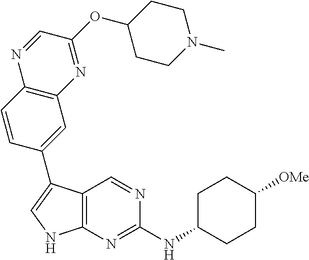 [SMILES: CO[C@H]1CC[C@@H](Nc2ncc3c(-c4ccc5ncc(OC6CCN(C)CC6)nc5c4)c[nH]c3n2)CC1] | 2017 |
| 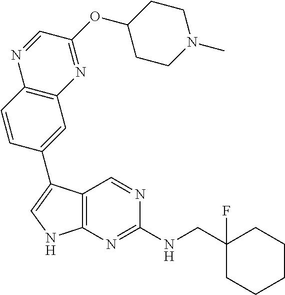 [SMILES: CN1CCC(Oc2cnc3ccc(-c4c[nH]c5nc(NCC6(F)CCCCC6)ncc45)cc3n2)CC1] | 2018 |
| 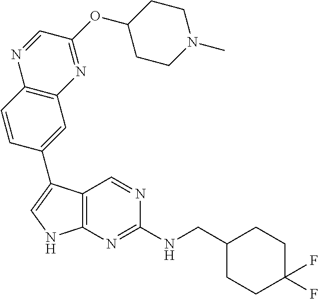 [SMILES: CN1CCC(Oc2cnc3ccc(-c4c[nH]c5nc(NCC6CCC(F)(F)CC6)ncc45)cc3n2)CC1] | 2019 |
| 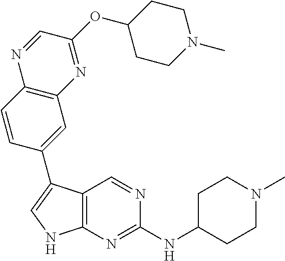 [SMILES: CN1CCC(Nc2ncc3c(-c4ccc5ncc(OC6CCN(C)CC6)nc5c4)c[nH]c3n2)CC1] | 2020 |
| 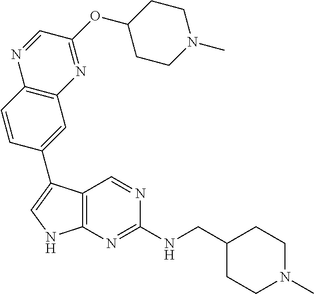 [SMILES: CN1CCC(CNc2ncc3c(-c4ccc5ncc(OC6CCN(C)CC6)nc5c4)c[nH]c3n2)CC1] | 2021 |
| 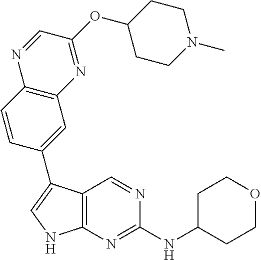 [SMILES: CN1CCC(Oc2cnc3ccc(-c4c[nH]c5nc(NC6CCOCC6)ncc45)cc3n2)CC1] | 2022 |
| 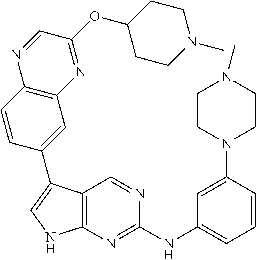 [SMILES: CN1CCC(Oc2cnc3ccc(-c4c[nH]c5nc(Nc6cccc(N7CCN(C)CC7)c6)ncc45)cc3n2)CC1] | 2023 |
| 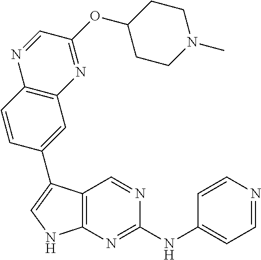 [SMILES: CN1CCC(Oc2cnc3ccc(-c4c[nH]c5nc(Nc6ccncc6)ncc45)cc3n2)CC1] | 2024 |
| 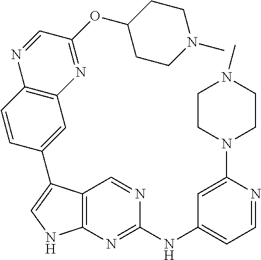 [SMILES: CN1CCC(Oc2cnc3ccc(-c4c[nH]c5nc(Nc6ccnc(N7CCN(C)CC7)c6)ncc45)cc3n2)CC1] | 2025 |
| 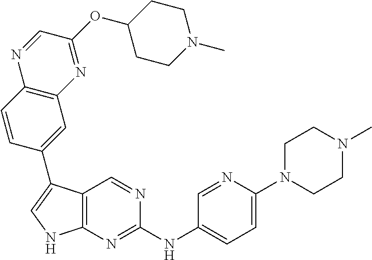 [SMILES: CN1CCC(Oc2cnc3ccc(-c4c[nH]c5nc(Nc6ccc(N7CCN(C)CC7)nc6)ncc45)cc3n2)CC1] | 2026 |
| 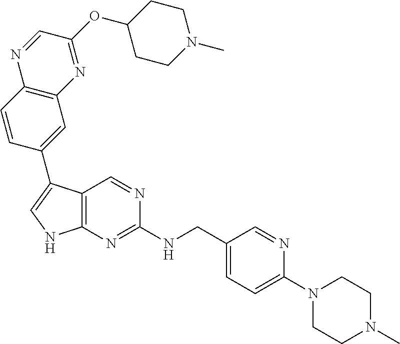 [SMILES: CN1CCC(Oc2cnc3ccc(-c4c[nH]c5nc(NCc6ccc(N7CCN(C)CC7)nc6)ncc45)cc3n2)CC1] | 2027 |
| 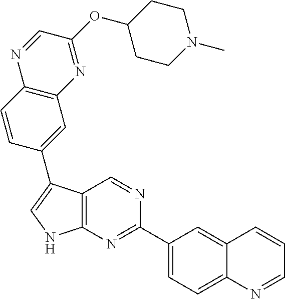 [SMILES: CN1CCC(Oc2cnc3ccc(-c4c[nH]c5nc(-c6ccc7ncccc7c6)ncc45)cc3n2)CC1] | 2028 |
| 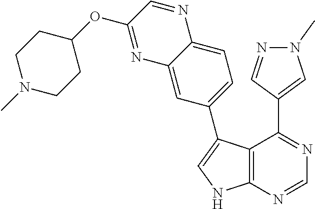 | 2029 |
| 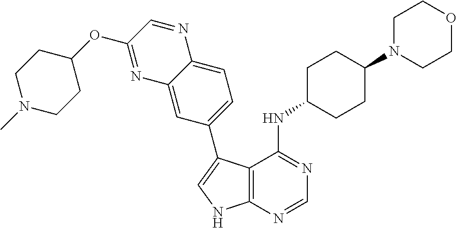 [SMILES: CN1CCC(Oc2cnc3ccc(-c4c[nH]c5ncnc(N[C@H]6CC[C@H](N7CCOCC7)CC6)c45)cc3n2)CC1] | 2030 |
| 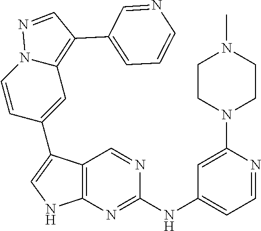 [SMILES: CN1CCN(c2cc(Nc3ncc4c(-c5ccn6ncc(-c7cccnc7)c6c5)c[nH]c4n3)ccn2)CC1] | 2031 |
| 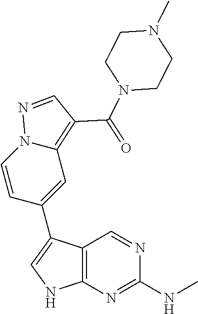 [SMILES: CNc1ncc2c(-c3ccn4ncc(C(=O)N5CCN(C)CC5)c4c3)c[nH]c2n1] | 2032 |
| 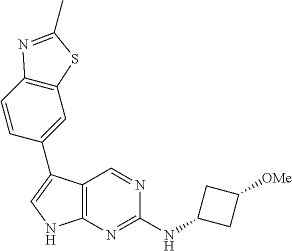 [SMILES: CO[C@H]1C[C@@H](Nc2ncc3c(-c4ccc5nc(C)sc5c4)c[nH]c3n2)C1] | 2033 |
| 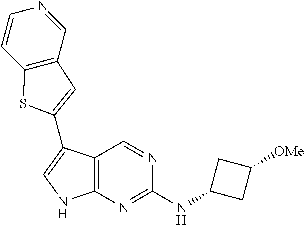 [SMILES: CO[C@H]1C[C@@H](Nc2ncc3c(-c4cc5cnccc5s4)c[nH]c3n2)C1] | 2034 |
| 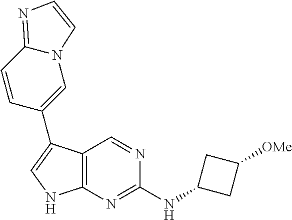 [SMILES: CO[C@H]1C[C@@H](Nc2ncc3c(-c4ccc5nccn5c4)c[nH]c3n2)C1] | 2035 |
| 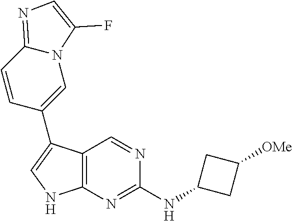 [SMILES: CO[C@H]1C[C@@H](Nc2ncc3c(-c4ccc5ncc(F)n5c4)c[nH]c3n2)C1] | 2036 |
| 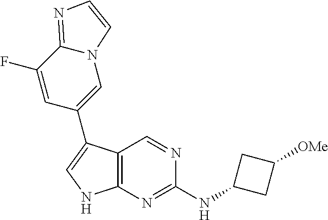 [SMILES: CO[C@H]1C[C@@H](Nc2ncc3c(-c4cc(F)c5nccn5c4)c[nH]c3n2)C1] | 2037 |
| 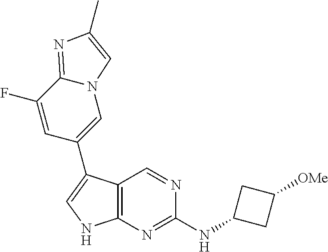 [SMILES: CO[C@H]1C[C@@H](Nc2ncc3c(-c4cc(F)c5nc(C)cn5c4)c[nH]c3n2)C1] | 2038 |
| 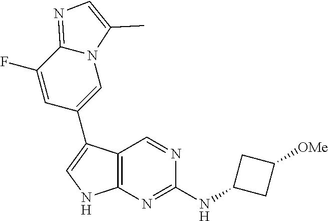 [SMILES: CO[C@H]1C[C@@H](Nc2ncc3c(-c4cc(F)c5ncc(C)n5c4)c[nH]c3n2)C1] | 2039 |
| 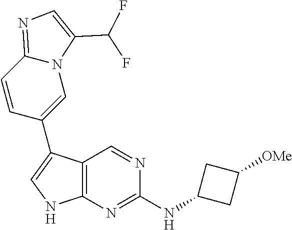 [SMILES: CO[C@H]1C[C@@H](Nc2ncc3c(-c4ccc5ncc(C(F)F)n5c4)c[nH]c3n2)C1] | 2040 |
| 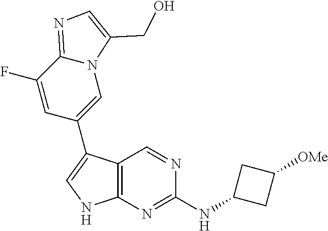 [SMILES: CO[C@H]1C[C@@H](Nc2ncc3c(-c4cc(F)c5ncc(CO)n5c4)c[nH]c3n2)C1] | 2041 |
| 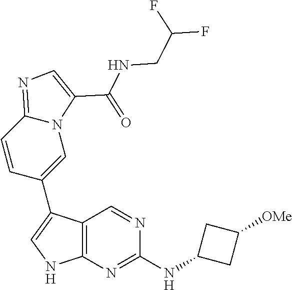 [SMILES: CO[C@H]1C[C@@H](Nc2ncc3c(-c4ccc5ncc(C(=O)NCC(F)F)n5c4)c[nH]c3n2)C1] | 2042 |
| 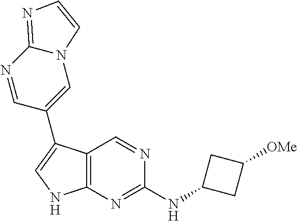 [SMILES: CO[C@H]1C[C@@H](Nc2ncc3c(-c4cnc5nccn5c4)c[nH]c3n2)C1] | 4043 |
| 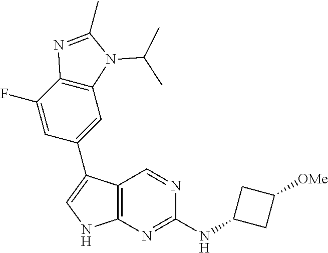 [SMILES: CO[C@H]1C[C@@H](Nc2ncc3c(-c4cc(F)c5nc(C)n(C(C)C)c5c4)c[nH]c3n2)C1] | 2044 |
| 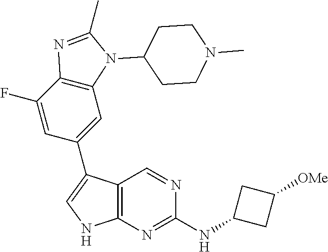 [SMILES: CO[C@H]1C[C@@H](Nc2ncc3c(-c4cc(F)c5nc(C)n(C6CCN(C)CC6)c5c4)c[nH]c3n2)C1] | 2045 |
| 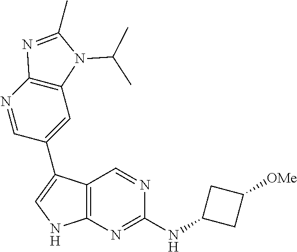 [SMILES: CO[C@H]1C[C@@H](Nc2ncc3c(-c4cnc5nc(C)n(C(C)C)c5c4)c[nH]c3n2)C1] | 2046 |
| 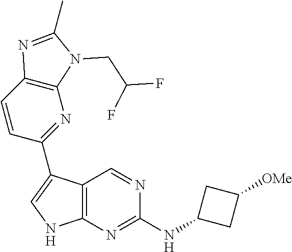 [SMILES: CO[C@H]1C[C@@H](Nc2ncc3c(-c4ccc5nc(C)n(CC(F)F)c5n4)c[nH]c3n2)C1] | 2047 |
| 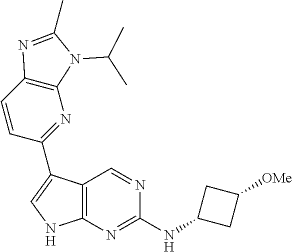 [SMILES: CO[C@H]1C[C@@H](Nc2ncc3c(-c4ccc5nc(C)n(C(C)C)c5n4)c[nH]c3n2)C1] | 2048 |
| 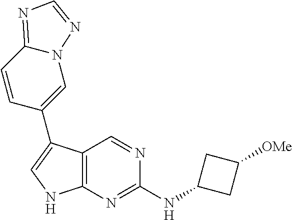 [SMILES: CO[C@H]1C[C@@H](Nc2ncc3c(-c4ccc5ncnn5c4)c[nH]c3n2)C1] | 2049 |
| 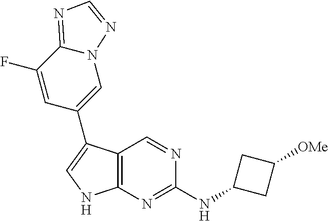 [SMILES: CO[C@H]1C[C@@H](Nc2ncc3c(-c4cc(F)c5ncnn5c4)c[nH]c3n2)C1] | 2050 |
| 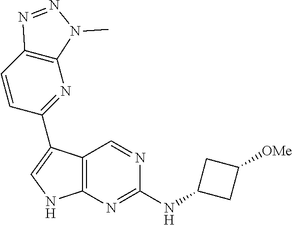 [SMILES: CO[C@H]1C[C@@H](Nc2ncc3c(-c4ccc5nnn(C)c5n4)c[nH]c3n2)C1] | 2051 |
| 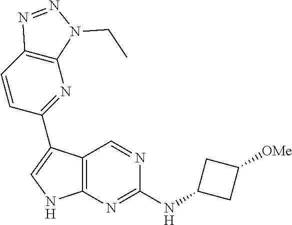 [SMILES: CCn1nnc2ccc(-c3c[nH]c4nc(N[C@H]5C[C@@H](OC)C5)ncc34)nc21] | 2052 |
| 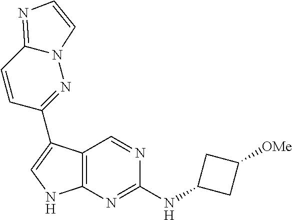 [SMILES: CO[C@H]1C[C@@H](Nc2ncc3c(-c4ccc5nccn5n4)c[nH]c3n2)C1] | 2053 |
| 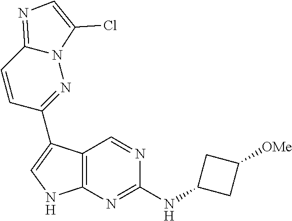 [SMILES: CO[C@H]1C[C@@H](Nc2ncc3c(-c4ccc5ncc(Cl)n5n4)c[nH]c3n2)C1] | 5054 |
| 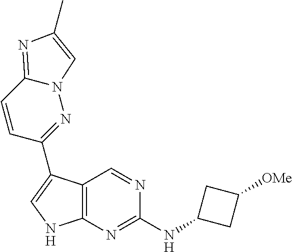 [SMILES: CO[C@H]1C[C@@H](Nc2ncc3c(-c4ccc5nc(C)cn5n4)c[nH]c3n2)C1] | 2055 |
| 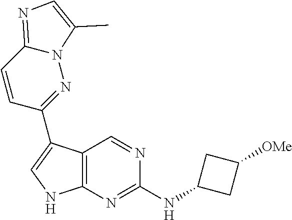 [SMILES: CO[C@H]1C[C@@H](Nc2ncc3c(-c4ccc5ncc(C)n5n4)c[nH]c3n2)C1] | 2056 |
| 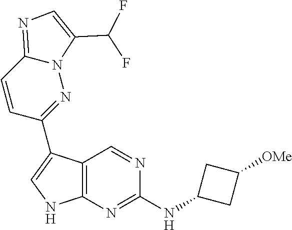 [SMILES: CO[C@H]1C[C@@H](Nc2ncc3c(-c4ccc5ncc(C(F)F)n5n4)c[nH]c3n2)C1] | 2057 |
| 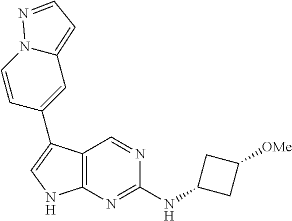 [SMILES: CO[C@H]1C[C@@H](Nc2ncc3c(-c4ccn5nccc5c4)c[nH]c3n2)C1] | 2058 |
| 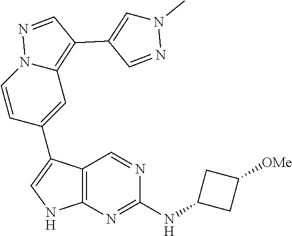 [SMILES: CO[C@H]1C[C@@H](Nc2ncc3c(-c4ccn5ncc(-c6cnn(C)c6)c5c4)c[nH]c3n2)C1] | 2059 |
| 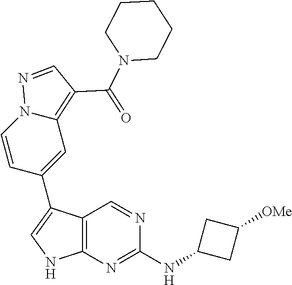 [SMILES: CO[C@H]1C[C@@H](Nc2ncc3c(-c4ccn5ncc(C(=O)N6CCCCC6)c5c4)c[nH]c3n2)C1] | 2060 |
| 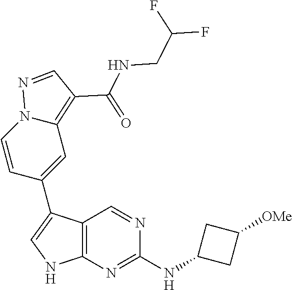 [SMILES: CO[C@H]1C[C@@H](Nc2ncc3c(-c4ccn5ncc(C(=O)NCC(F)F)c5c4)c[nH]c3n2)C1] | 2061 |
| 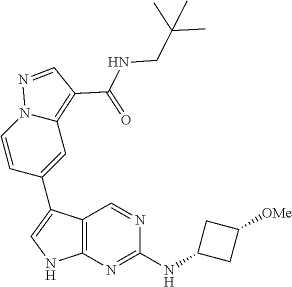 [SMILES: CO[C@H]1C[C@@H](Nc2ncc3c(-c4ccn5ncc(C(=O)NCC(C)(C)C)c5c4)c[nH]c3n2)C1] | 2062 |
| 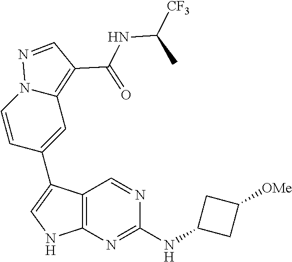 [SMILES: CO[C@H]1C[C@@H](Nc2ncc3c(-c4ccn5ncc(C(=O)N[C@H](C)C(F)(F)F)c5c4)c[nH]c3n2)C1] | 2063 |
| 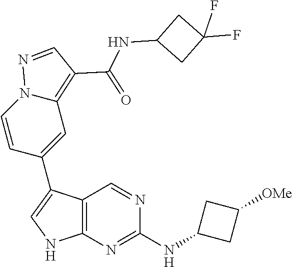 [SMILES: CO[C@H]1C[C@@H](Nc2ncc3c(-c4ccn5ncc(C(=O)NC6CC(F)(F)C6)c5c4)c[nH]c3n2)C1] | 2064 |
| 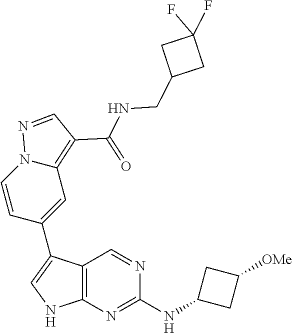 [SMILES: CO[C@H]1C[C@@H](Nc2ncc3c(-c4ccn5ncc(C(=O)NCC6CC(F)(F)C6)c5c4)c[nH]c3n2)C1] | 2065 |
| 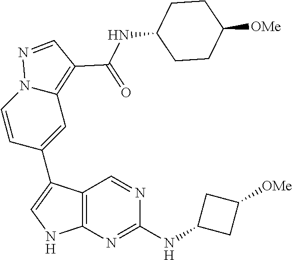 [SMILES: CO[C@H]1CC[C@H](NC(=O)c2cnn3ccc(-c4c[nH]c5nc(N[C@H]6C[C@@H](OC)C6)ncc45)cc23)CC1] | 2066 |
| 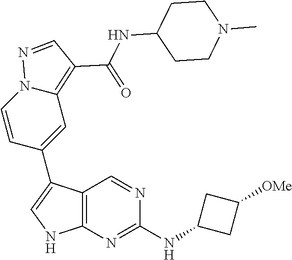 [SMILES: CO[C@H]1C[C@@H](Nc2ncc3c(-c4ccn5ncc(C(=O)NC6CCN(C)CC6)c5c4)c[nH]c3n2)C1] | 2067 |
| 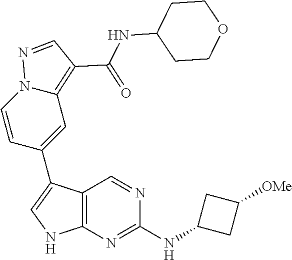 [SMILES: CO[C@H]1C[C@@H](Nc2ncc3c(-c4ccn5ncc(C(=O)NC6CCOCC6)c5c4)c[nH]c3n2)C1] | 2068 |
| 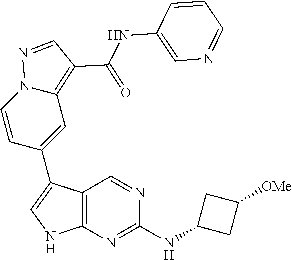 [SMILES: CO[C@H]1C[C@@H](Nc2ncc3c(-c4ccn5ncc(C(=O)Nc6cccnc6)c5c4)c[nH]c3n2)C1] | 2069 |
| 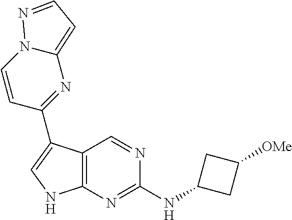 [SMILES: CO[C@H]1C[C@@H](Nc2ncc3c(-c4ccn5nccc5n4)c[nH]c3n2)C1] | 2070 |
| 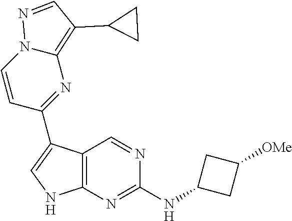 [SMILES: CO[C@H]1C[C@@H](Nc2ncc3c(-c4ccn5ncc(C6CC6)c5n4)c[nH]c3n2)C1] | 2071 |
| 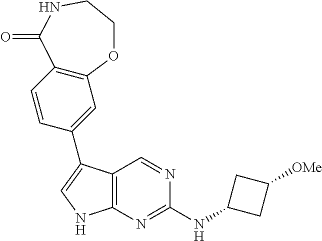 [SMILES: CO[C@H]1C[C@@H](Nc2ncc3c(-c4ccc5c(c4)OCCNC5=O)c[nH]c3n2)C1] | 2072 |
| 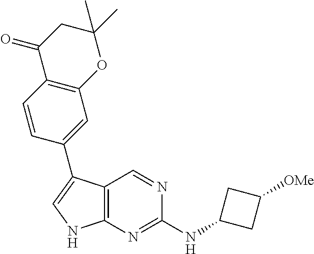 [SMILES: CO[C@H]1C[C@@H](Nc2ncc3c(-c4ccc5c(c4)OC(C)(C)CC5=O)c[nH]c3n2)C1] | 2073 |
| 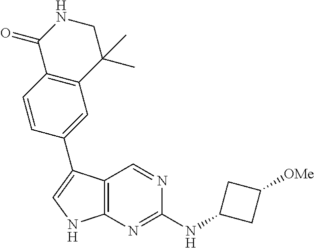 [SMILES: CO[C@H]1C[C@@H](Nc2ncc3c(-c4ccc5c(c4)C(C)(C)CNC5=O)c[nH]c3n2)C1] | 2074 |
| 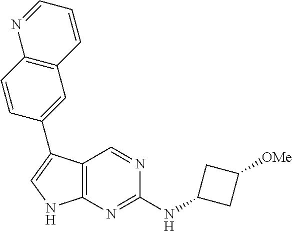 [SMILES: CO[C@H]1C[C@@H](Nc2ncc3c(-c4ccc5ncccc5c4)c[nH]c3n2)C1] | 2075 |
| 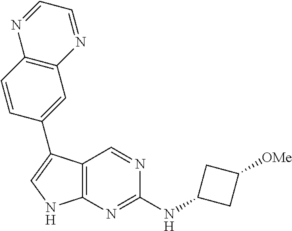 [SMILES: CO[C@H]1C[C@@H](Nc2ncc3c(-c4ccc5nccnc5c4)c[nH]c3n2)C1] | 2076 |
| 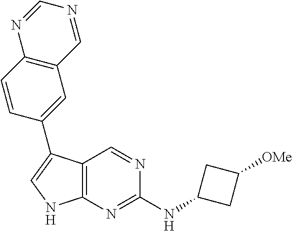 [SMILES: CO[C@H]1C[C@@H](Nc2ncc3c(-c4ccc5ncncc5c4)c[nH]c3n2)C1] | 2077 |
| 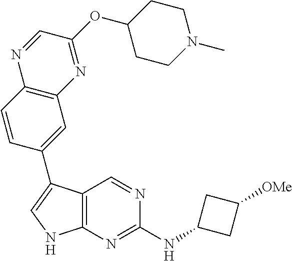 [SMILES: CO[C@H]1C[C@@H](Nc2ncc3c(-c4ccc5ncc(OC6CCN(C)CC6)nc5c4)c[nH]c3n2)C1] | 2078 |
| 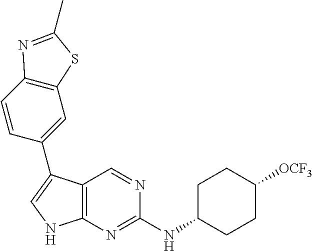 [SMILES: Cc1nc2ccc(-c3c[nH]c4nc(N[C@H]5CC[C@@H](OC(F)(F)F)CC5)ncc34)cc2s1] | 2079 |
| 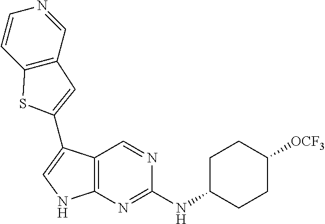 [SMILES: FC(F)(F)O[C@H]1CC[C@@H](Nc2ncc3c(-c4cc5cnccc5s4)c[nH]c3n2)CC1] | 2080 |
| 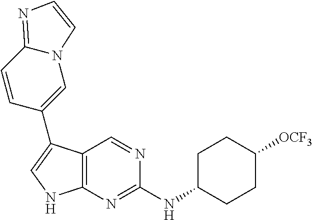 [SMILES: FC(F)(F)O[C@H]1CC[C@@H](Nc2ncc3c(-c4ccc5nccn5c4)c[nH]c3n2)CC1] | 2081 |
| 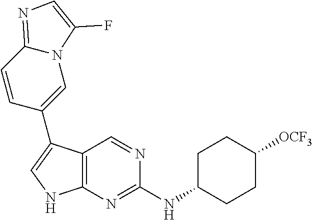 [SMILES: Fc1cnc2ccc(-c3c[nH]c4nc(N[C@H]5CC[C@@H](OC(F)(F)F)CC5)ncc34)cn12] | 2082 |
| 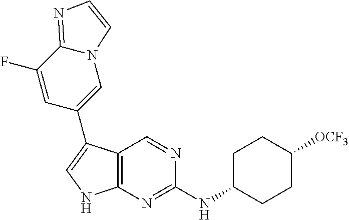 [SMILES: Fc1cc(-c2c[nH]c3nc(N[C@H]4CC[C@@H](OC(F)(F)F)CC4)ncc23)cn2ccnc12] | 2083 |
| 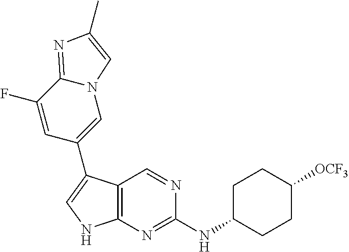 [SMILES: Cc1cn2cc(-c3c[nH]c4nc(N[C@H]5CC[C@@H](OC(F)(F)F)CC5)ncc34)cc(F)c2n1] | 2084 |
| 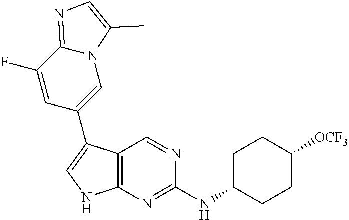 [SMILES: Cc1cnc2c(F)cc(-c3c[nH]c4nc(N[C@H]5CC[C@@H](OC(F)(F)F)CC5)ncc34)cn12] | 2085 |
| 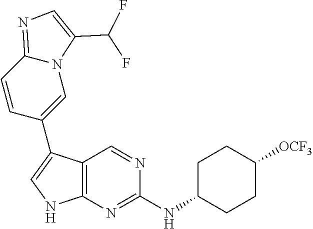 [SMILES: FC(F)c1cnc2ccc(-c3c[nH]c4nc(N[C@H]5CC[C@@H](OC(F)(F)F)CC5)ncc34)cn12] | 2086 |
| 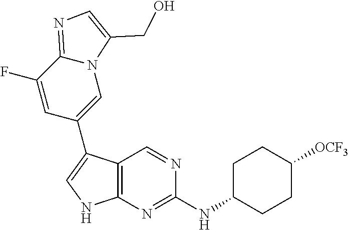 [SMILES: OCc1cnc2c(F)cc(-c3c[nH]c4nc(N[C@H]5CC[C@@H](OC(F)(F)F)CC5)ncc34)cn12] | 2087 |
| 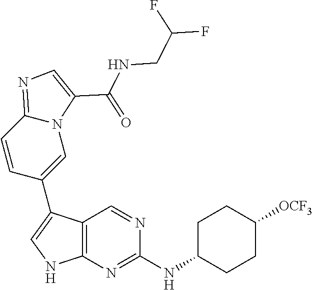 [SMILES: O=C(NCC(F)F)c1cnc2ccc(-c3c[nH]c4nc(N[C@H]5CC[C@@H](OC(F)(F)F)CC5)ncc34)cn12] | 2088 |
| 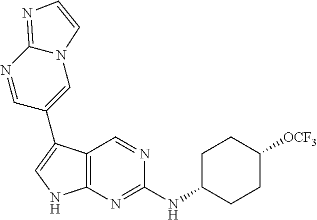 [SMILES: FC(F)(F)O[C@H]1CC[C@@H](Nc2ncc3c(-c4cnc5nccn5c4)c[nH]c3n2)CC1] | 2089 |
| 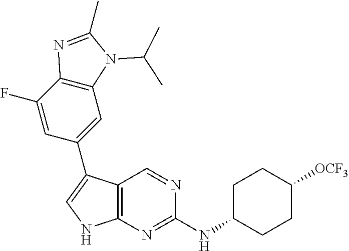 [SMILES: Cc1nc2c(F)cc(-c3c[nH]c4nc(N[C@H]5CC[C@@H](OC(F)(F)F)CC5)ncc34)cc2n1C(C)C] | 2090 |
| 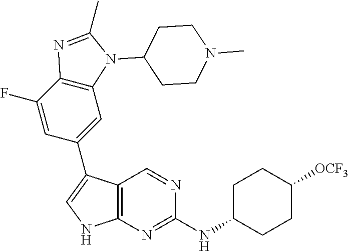 [SMILES: Cc1nc2c(F)cc(-c3c[nH]c4nc(N[C@H]5CC[C@@H](OC(F)(F)F)CC5)ncc34)cc2n1C1CCN(C)CC1] | 2091 |
| 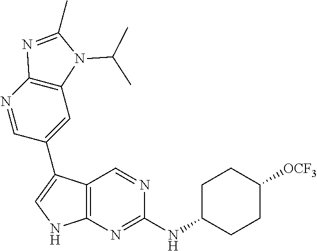 [SMILES: Cc1nc2ncc(-c3c[nH]c4nc(N[C@H]5CC[C@@H](OC(F)(F)F)CC5)ncc34)cc2n1C(C)C] | 2092 |
| 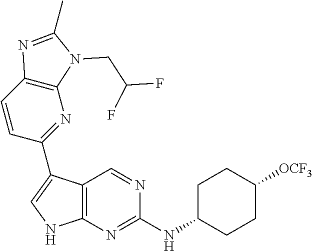 [SMILES: Cc1nc2ccc(-c3c[nH]c4nc(N[C@H]5CC[C@@H](OC(F)(F)F)CC5)ncc34)nc2n1CC(F)F] | 2093 |
| 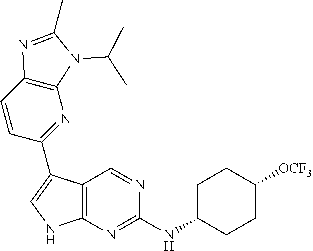 [SMILES: Cc1nc2ccc(-c3c[nH]c4nc(N[C@H]5CC[C@@H](OC(F)(F)F)CC5)ncc34)nc2n1C(C)C] | 2094 |
| 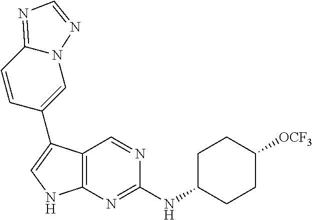 [SMILES: FC(F)(F)O[C@H]1CC[C@@H](Nc2ncc3c(-c4ccc5ncnn5c4)c[nH]c3n2)CC1] | 2095 |
| 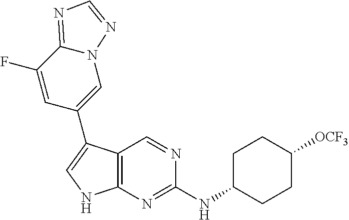 [SMILES: Fc1cc(-c2c[nH]c3nc(N[C@H]4CC[C@@H](OC(F)(F)F)CC4)ncc23)cn2ncnc12] | 2096 |
| 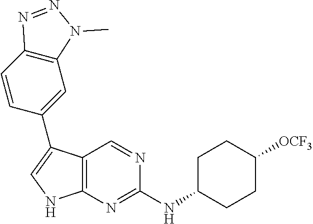 [SMILES: Cn1nnc2ccc(-c3c[nH]c4nc(N[C@H]5CC[C@@H](OC(F)(F)F)CC5)ncc34)cc21] | 2097 |
| 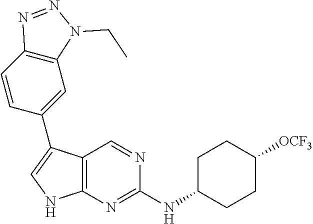 [SMILES: CCn1nnc2ccc(-c3c[nH]c4nc(N[C@H]5CC[C@@H](OC(F)(F)F)CC5)ncc34)cc21] | 2098 |
| 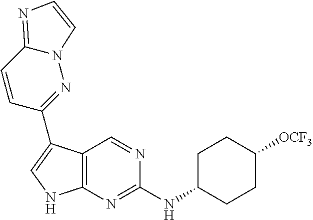 [SMILES: FC(F)(F)O[C@H]1CC[C@@H](Nc2ncc3c(-c4ccc5nccn5n4)c[nH]c3n2)CC1] | 2099 |
| 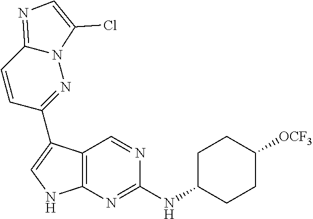 [SMILES: FC(F)(F)O[C@H]1CC[C@@H](Nc2ncc3c(-c4ccc5ncc(Cl)n5n4)c[nH]c3n2)CC1] | 2100 |
| 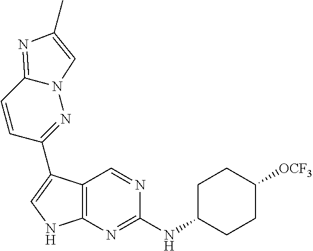 [SMILES: Cc1cn2nc(-c3c[nH]c4nc(N[C@H]5CC[C@@H](OC(F)(F)F)CC5)ncc34)ccc2n1] | 2101 |
| 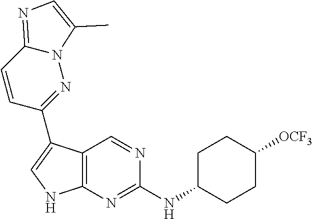 [SMILES: Cc1cnc2ccc(-c3c[nH]c4nc(N[C@H]5CC[C@@H](OC(F)(F)F)CC5)ncc34)nn12] | 2102 |
| 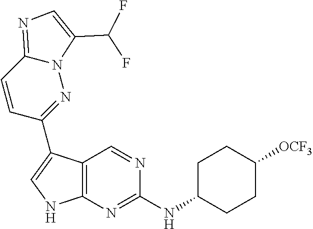 [SMILES: FC(F)c1cnc2ccc(-c3c[nH]c4nc(N[C@H]5CC[C@@H](OC(F)(F)F)CC5)ncc34)nn12] | 2103 |
| 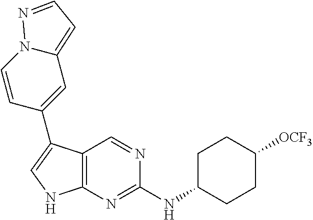 [SMILES: FC(F)(F)O[C@H]1CC[C@@H](Nc2ncc3c(-c4ccn5nccc5c4)c[nH]c3n2)CC1] | 2104 |
| 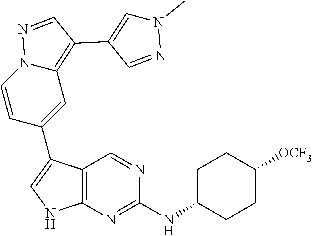 [SMILES: Cn1cc(-c2cnn3ccc(-c4c[nH]c5nc(N[C@H]6CC[C@@H](OC(F)(F)F)CC6)ncc45)cc23)cn1] | 2105 |
| 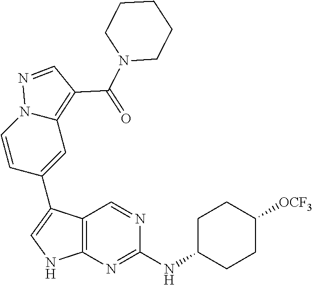 [SMILES: O=C(c1cnn2ccc(-c3c[nH]c4nc(N[C@H]5CC[C@@H](OC(F)(F)F)CC5)ncc34)cc12)N1CCCCC1] | 2106 |
| 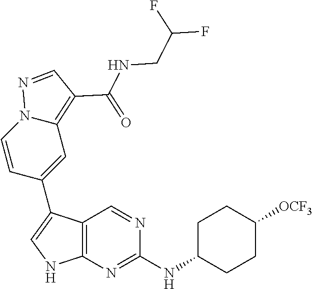 [SMILES: O=C(NCC(F)F)c1cnn2ccc(-c3c[nH]c4nc(N[C@H]5CC[C@@H](OC(F)(F)F)CC5)ncc34)cc12] | 2107 |
| 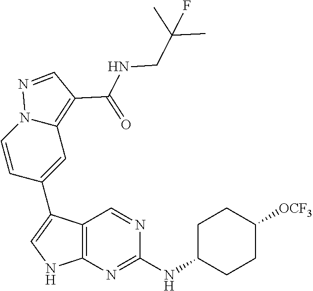 [SMILES: CC(C)(F)CNC(=O)c1cnn2ccc(-c3c[nH]c4nc(N[C@H]5CC[C@@H](OC(F)(F)F)CC5)ncc34)cc12] | 2108 |
| 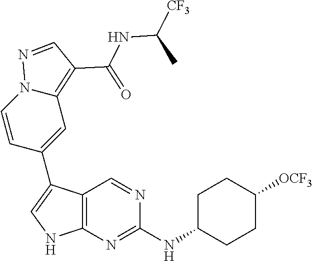 [SMILES: C[C@@H](NC(=O)c1cnn2ccc(-c3c[nH]c4nc(N[C@H]5CC[C@@H](OC(F)(F)F)CC5)ncc34)cc12)C(F)(F)F] | 2109 |
| 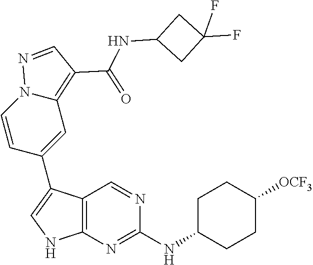 [SMILES: O=C(NC1CC(F)(F)C1)c1cnn2ccc(-c3c[nH]c4nc(N[C@H]5CC[C@@H](OC(F)(F)F)CC5)ncc34)cc12] | 2110 |
| 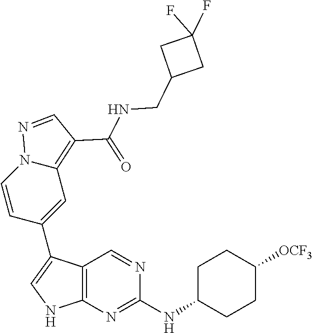 [SMILES: O=C(NCC1CC(F)(F)C1)c1cnn2ccc(-c3c[nH]c4nc(N[C@H]5CC[C@@H](OC(F)(F)F)CC5)ncc34)cc12] | 2111 |
| 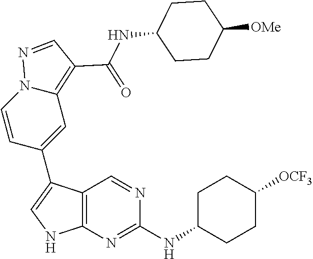 [SMILES: CO[C@H]1CC[C@H](NC(=O)c2cnn3ccc(-c4c[nH]c5nc(N[C@H]6CC[C@@H](OC(F)(F)F)CC6)ncc45)cc23)CC1] | 2112 |
| 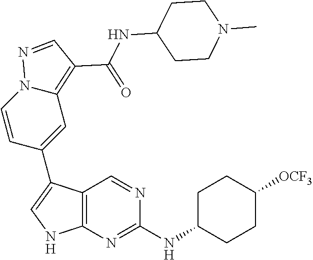 [SMILES: CN1CCC(NC(=O)c2cnn3ccc(-c4c[nH]c5nc(N[C@H]6CC[C@@H](OC(F)(F)F)CC6)ncc45)cc23)CC1] | 2113 |
| 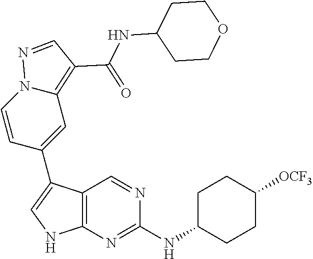 [SMILES: O=C(NC1CCOCC1)c1cnn2ccc(-c3c[nH]c4nc(N[C@H]5CC[C@@H](OC(F)(F)F)CC5)ncc34)cc12] | 2114 |
| 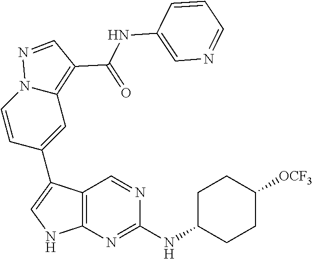 [SMILES: O=C(Nc1cccnc1)c1cnn2ccc(-c3c[nH]c4nc(N[C@H]5CC[C@@H](OC(F)(F)F)CC5)ncc34)cc12] | 2115 |
| 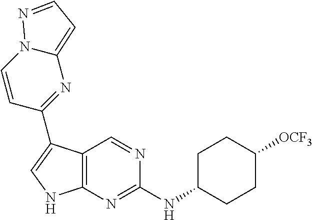 [SMILES: FC(F)(F)O[C@H]1CC[C@@H](Nc2ncc3c(-c4ccn5nccc5n4)c[nH]c3n2)CC1] | 2116 |
| 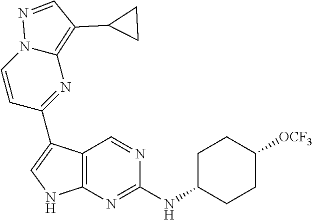 [SMILES: FC(F)(F)O[C@H]1CC[C@@H](Nc2ncc3c(-c4ccn5ncc(C6CC6)c5n4)c[nH]c3n2)CC1] | 2117 |
| 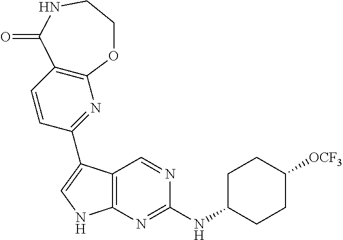 [SMILES: O=C1NCCOc2nc(-c3c[nH]c4nc(N[C@H]5CC[C@@H](OC(F)(F)F)CC5)ncc34)ccc21] | 2118 |
| 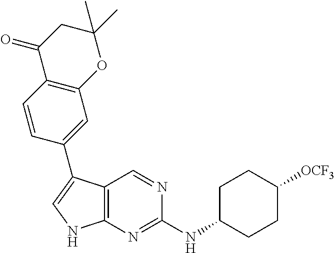 [SMILES: CC1(C)CC(=O)c2ccc(-c3c[nH]c4nc(N[C@H]5CC[C@@H](OC(F)(F)F)CC5)ncc34)cc2O1] | 2119 |
| 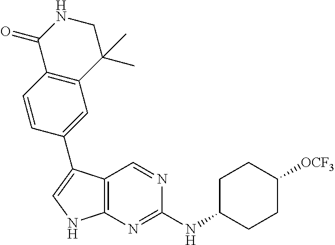 [SMILES: CC1(C)CNC(=O)c2ccc(-c3c[nH]c4nc(N[C@H]5CC[C@@H](OC(F)(F)F)CC5)ncc34)cc21] | 2120 |
| 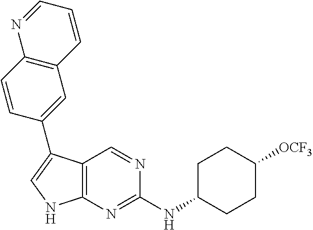 [SMILES: FC(F)(F)O[C@H]1CC[C@@H](Nc2ncc3c(-c4ccc5ncccc5c4)c[nH]c3n2)CC1] | 2121 |
| 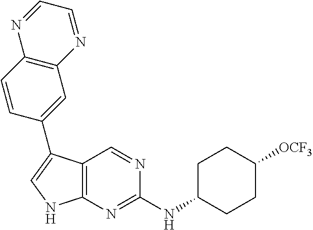 [SMILES: FC(F)(F)O[C@H]1CC[C@@H](Nc2ncc3c(-c4ccc5nccnc5c4)c[nH]c3n2)CC1] | 2122 |
| 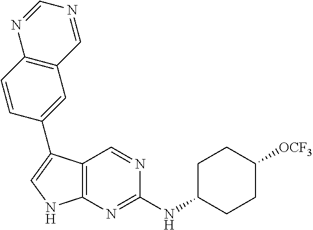 [SMILES: FC(F)(F)O[C@H]1CC[C@@H](Nc2ncc3c(-c4ccc5ncncc5c4)c[nH]c3n2)CC1] | 2123 |
| 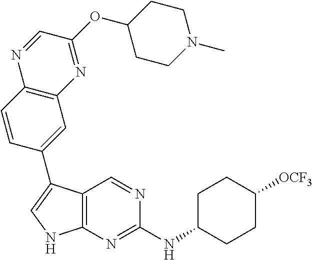 [SMILES: CN1CCC(Oc2cnc3ccc(-c4c[nH]c5nc(N[C@H]6CC[C@@H](OC(F)(F)F)CC6)ncc45)cc3n2)CC1] | 2124 |
| 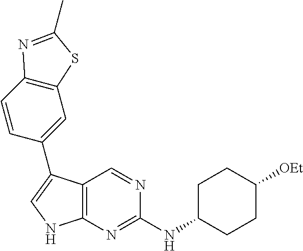 [SMILES: CCO[C@H]1CC[C@@H](Nc2ncc3c(-c4ccc5nc(C)sc5c4)c[nH]c3n2)CC1] | 2125 |
| 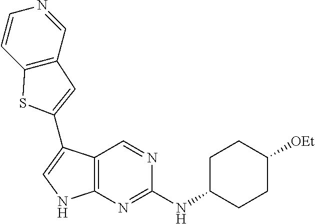 [SMILES: CCO[C@H]1CC[C@@H](Nc2ncc3c(-c4cc5cnccc5s4)c[nH]c3n2)CC1] | 2126 |
| 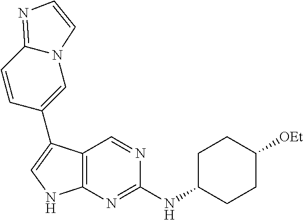 [SMILES: CCO[C@H]1CC[C@@H](Nc2ncc3c(-c4ccc5nccn5c4)c[nH]c3n2)CC1] | 2127 |
| 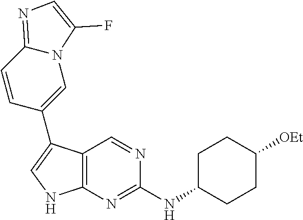 [SMILES: CCO[C@H]1CC[C@@H](Nc2ncc3c(-c4ccc5ncc(F)n5c4)c[nH]c3n2)CC1] | 2128 |
| 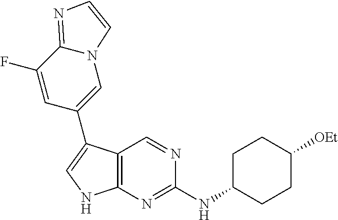 [SMILES: CCO[C@H]1CC[C@@H](Nc2ncc3c(-c4cc(F)c5nccn5c4)c[nH]c3n2)CC1] | 2129 |
| 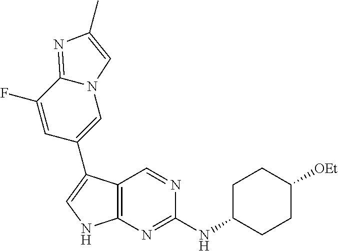 [SMILES: CCO[C@H]1CC[C@@H](Nc2ncc3c(-c4cc(F)c5nc(C)cn5c4)c[nH]c3n2)CC1] | 2130 |
| 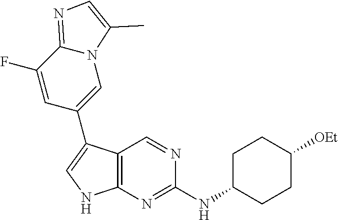 [SMILES: CCO[C@H]1CC[C@@H](Nc2ncc3c(-c4cc(F)c5ncc(C)n5c4)c[nH]c3n2)CC1] | 2131 |
| 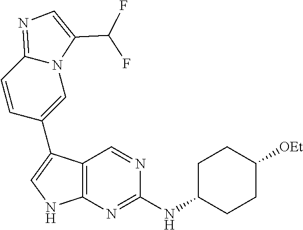 [SMILES: CCO[C@H]1CC[C@@H](Nc2ncc3c(-c4ccc5ncc(C(F)F)n5c4)c[nH]c3n2)CC1] | 2132 |
| 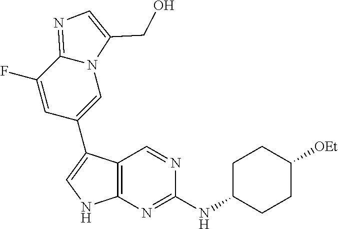 [SMILES: CCO[C@H]1CC[C@@H](Nc2ncc3c(-c4cc(F)c5ncc(CO)n5c4)c[nH]c3n2)CC1] | 2133 |
| 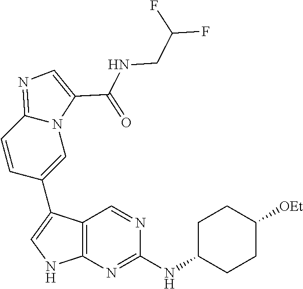 [SMILES: CCO[C@H]1CC[C@@H](Nc2ncc3c(-c4ccc5ncc(C(=O)NCC(F)F)n5c4)c[nH]c3n2)CC1] | 2134 |
| 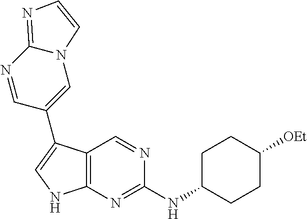 [SMILES: CCO[C@H]1CC[C@@H](Nc2ncc3c(-c4cnc5nccn5c4)c[nH]c3n2)CC1] | 2135 |
| 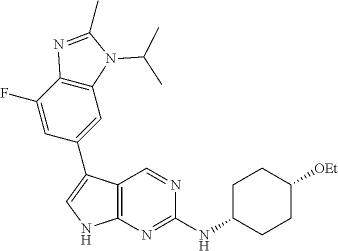 [SMILES: CCO[C@H]1CC[C@@H](Nc2ncc3c(-c4cc(F)c5nc(C)n(C(C)C)c5c4)c[nH]c3n2)CC1] | 2136 |
| 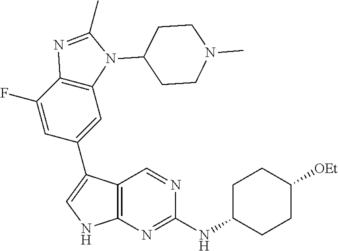 [SMILES: CCO[C@H]1CC[C@@H](Nc2ncc3c(-c4cc(F)c5nc(C)n(C6CCN(C)CC6)c5c4)c[nH]c3n2)CC1] | 2137 |
| 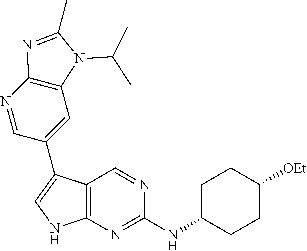 [SMILES: CCO[C@H]1CC[C@@H](Nc2ncc3c(-c4cnc5nc(C)n(C(C)C)c5c4)c[nH]c3n2)CC1] | 2138 |
| 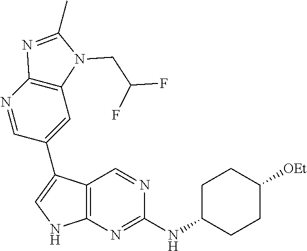 [SMILES: CCO[C@H]1CC[C@@H](Nc2ncc3c(-c4cnc5nc(C)n(CC(F)F)c5c4)c[nH]c3n2)CC1] | 2139 |
| 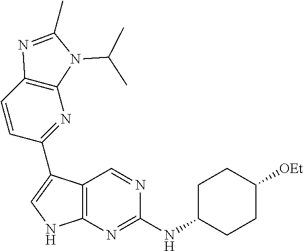 [SMILES: CCO[C@H]1CC[C@@H](Nc2ncc3c(-c4ccc5nc(C)n(C(C)C)c5n4)c[nH]c3n2)CC1] | 2140 |
| 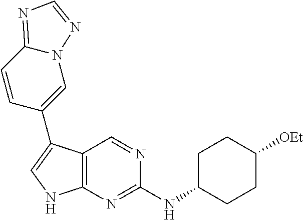 [SMILES: CCO[C@H]1CC[C@@H](Nc2ncc3c(-c4ccc5ncnn5c4)c[nH]c3n2)CC1] | 2141 |
| 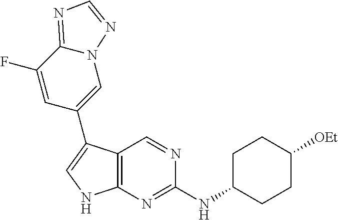 [SMILES: CCO[C@H]1CC[C@@H](Nc2ncc3c(-c4cc(F)c5ncnn5c4)c[nH]c3n2)CC1] | 2142 |
| 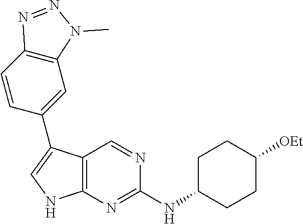 [SMILES: CCO[C@H]1CC[C@@H](Nc2ncc3c(-c4ccc5nnn(C)c5c4)c[nH]c3n2)CC1] | 2143 |
| 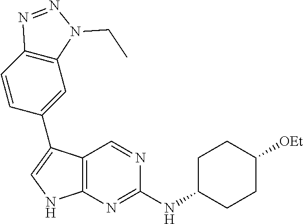 [SMILES: CCO[C@H]1CC[C@@H](Nc2ncc3c(-c4ccc5nnn(CC)c5c4)c[nH]c3n2)CC1] | 2144 |
| 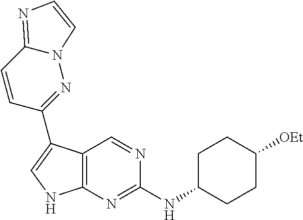 [SMILES: CCO[C@H]1CC[C@@H](Nc2ncc3c(-c4ccc5nccn5n4)c[nH]c3n2)CC1] | 2145 |
| 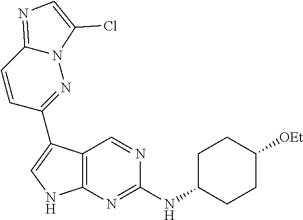 [SMILES: CCO[C@H]1CC[C@@H](Nc2ncc3c(-c4ccc5ncc(Cl)n5n4)c[nH]c3n2)CC1] | 2146 |
| 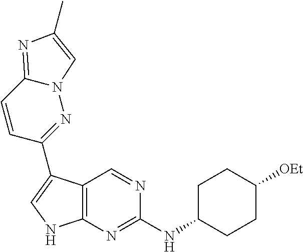 [SMILES: CCO[C@H]1CC[C@@H](Nc2ncc3c(-c4ccc5nc(C)cn5n4)c[nH]c3n2)CC1] | 2147 |
| 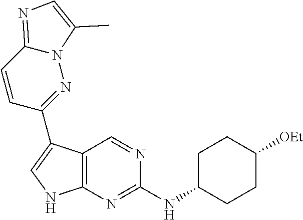 [SMILES: CCO[C@H]1CC[C@@H](Nc2ncc3c(-c4ccc5ncc(C)n5n4)c[nH]c3n2)CC1] | 2148 |
| 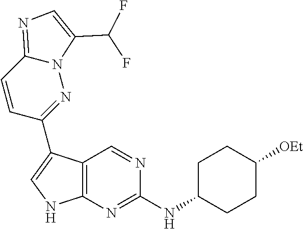 [SMILES: CCO[C@H]1CC[C@@H](Nc2ncc3c(-c4ccc5ncc(C(F)F)n5n4)c[nH]c3n2)CC1] | 2149 |
| 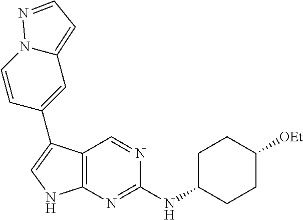 [SMILES: CCO[C@H]1CC[C@@H](Nc2ncc3c(-c4ccn5nccc5c4)c[nH]c3n2)CC1] | 2150 |
| 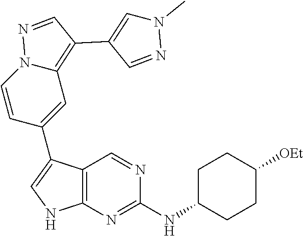 [SMILES: CCO[C@H]1CC[C@@H](Nc2ncc3c(-c4ccn5ncc(-c6cnn(C)c6)c5c4)c[nH]c3n2)CC1] | 2151 |
| 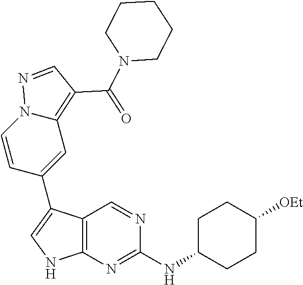 [SMILES: CCO[C@H]1CC[C@@H](Nc2ncc3c(-c4ccn5ncc(C(=O)N6CCCCC6)c5c4)c[nH]c3n2)CC1] | 2152 |
| 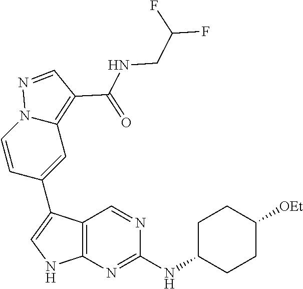 [SMILES: CCO[C@H]1CC[C@@H](Nc2ncc3c(-c4ccn5ncc(C(=O)NCC(F)F)c5c4)c[nH]c3n2)CC1] | 2153 |
| 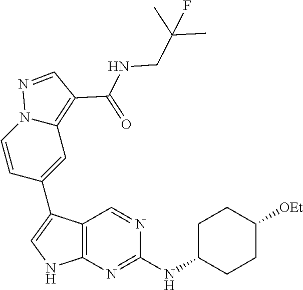 [SMILES: CCO[C@H]1CC[C@@H](Nc2ncc3c(-c4ccn5ncc(C(=O)NCC(C)(C)F)c5c4)c[nH]c3n2)CC1] | 2154 |
| 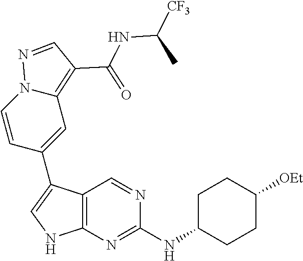 [SMILES: CCO[C@H]1CC[C@@H](Nc2ncc3c(-c4ccn5ncc(C(=O)N[C@H](C)C(F)(F)F)c5c4)c[nH]c3n2)CC1] | 2155 |
| 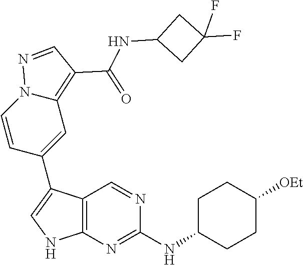 [SMILES: CCO[C@H]1CC[C@@H](Nc2ncc3c(-c4ccn5ncc(C(=O)NC6CC(F)(F)C6)c5c4)c[nH]c3n2)CC1] | 2156 |
| 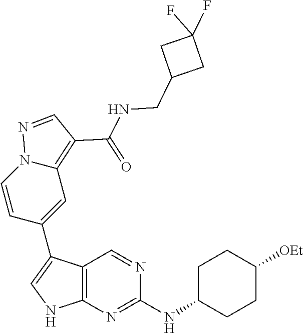 [SMILES: CCO[C@H]1CC[C@@H](Nc2ncc3c(-c4ccn5ncc(C(=O)NCC6CC(F)(F)C6)c5c4)c[nH]c3n2)CC1] | 2157 |
| 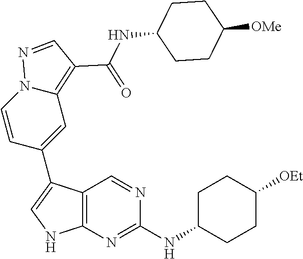 [SMILES: CCO[C@H]1CC[C@@H](Nc2ncc3c(-c4ccn5ncc(C(=O)N[C@H]6CC[C@H](OC)CC6)c5c4)c[nH]c3n2)CC1] | 2158 |
| 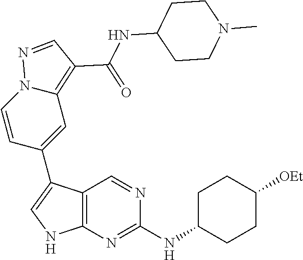 [SMILES: CCO[C@H]1CC[C@@H](Nc2ncc3c(-c4ccn5ncc(C(=O)NC6CCN(C)CC6)c5c4)c[nH]c3n2)CC1] | 2159 |
| 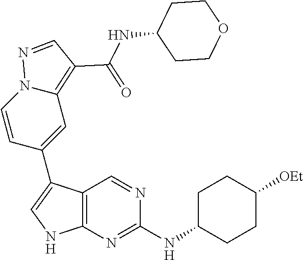 | 2160 |
| 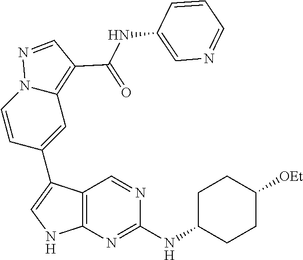 | 2161 |
| 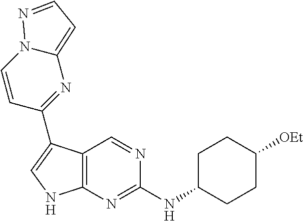 [SMILES: CCO[C@H]1CC[C@@H](Nc2ncc3c(-c4ccn5nccc5n4)c[nH]c3n2)CC1] | 2162 |
| 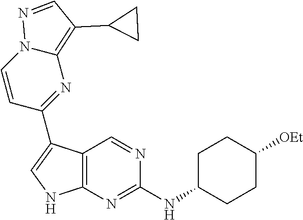 [SMILES: CCO[C@H]1CC[C@@H](Nc2ncc3c(-c4ccn5ncc(C6CC6)c5n4)c[nH]c3n2)CC1] | 2163 |
| 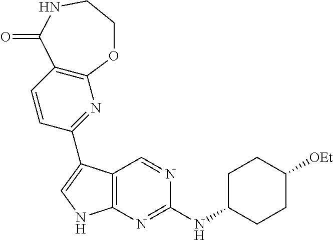 [SMILES: CCO[C@H]1CC[C@@H](Nc2ncc3c(-c4ccc5c(n4)OCCNC5=O)c[nH]c3n2)CC1] | 2164 |
| 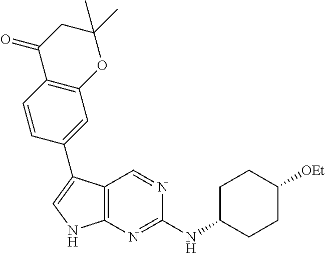 [SMILES: CCO[C@H]1CC[C@@H](Nc2ncc3c(-c4ccc5c(c4)OC(C)(C)CC5=O)c[nH]c3n2)CC1] | 2165 |
| 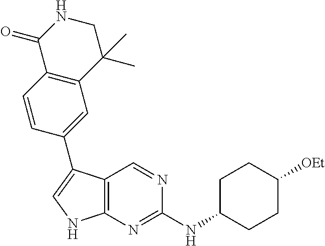 [SMILES: CCO[C@H]1CC[C@@H](Nc2ncc3c(-c4ccc5c(c4)C(C)(C)CNC5=O)c[nH]c3n2)CC1] | 2166 |
| 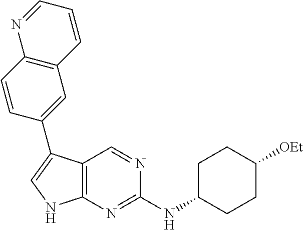 [SMILES: CCO[C@H]1CC[C@@H](Nc2ncc3c(-c4ccc5ncccc5c4)c[nH]c3n2)CC1] | 2167 |
| 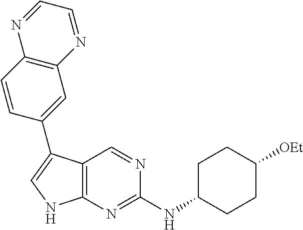 [SMILES: CCO[C@H]1CC[C@@H](Nc2ncc3c(-c4ccc5nccnc5c4)c[nH]c3n2)CC1] | 2168 |
| 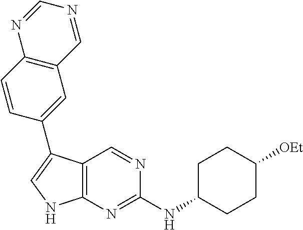 [SMILES: CCO[C@H]1CC[C@@H](Nc2ncc3c(-c4ccc5ncncc5c4)c[nH]c3n2)CC1] | 2169 |
| 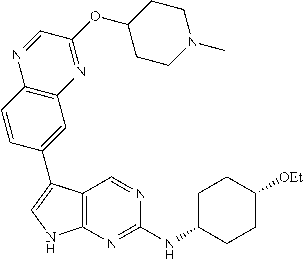 [SMILES: CCO[C@H]1CC[C@@H](Nc2ncc3c(-c4ccc5ncc(OC6CCN(C)CC6)nc5c4)c[nH]c3n2)CC1] | 2170 |
| 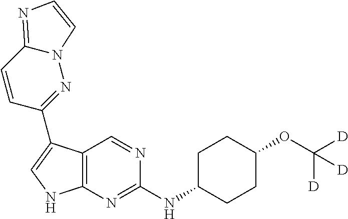 [SMILES: [2H]C([2H])([2H])O[C@H]1CC[C@@H](Nc2ncc3c(-c4ccc5nccn5n4)c[nH]c3n2)CC1] | 2171 |
| 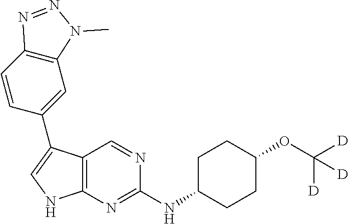 [SMILES: [2H]C([2H])([2H])O[C@H]1CC[C@@H](Nc2ncc3c(-c4ccc5nnn(C)c5c4)c[nH]c3n2)CC1] | 2172 |
| 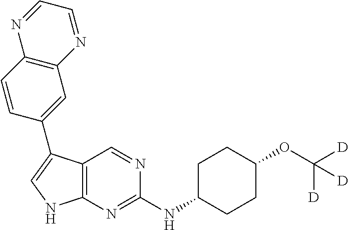 [SMILES: [2H]C([2H])([2H])O[C@H]1CC[C@@H](Nc2ncc3c(-c4ccc5nccnc5c4)c[nH]c3n2)CC1] | 2173 |
| 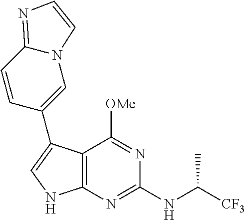 [SMILES: COc1nc(N[C@H](C)C(F)(F)F)nc2[nH]cc(-c3ccc4nccn4c3)c12] | 2174 |
| 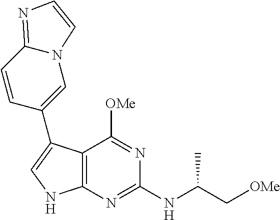 [SMILES: COC[C@@H](C)Nc1nc(OC)c2c(-c3ccc4nccn4c3)c[nH]c2n1] | 2175 |
| 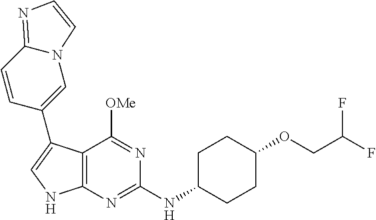 [SMILES: COc1nc(N[C@H]2CC[C@@H](OCC(F)F)CC2)nc2[nH]cc(-c3ccc4nccn4c3)c12] | 2176 |
| 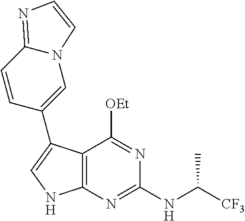 [SMILES: CCOc1nc(N[C@H](C)C(F)(F)F)nc2[nH]cc(-c3ccc4nccn4c3)c12] | 2177 |
| 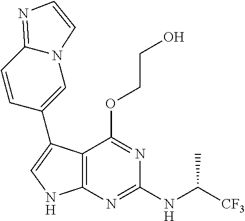 [SMILES: C[C@@H](Nc1nc(OCCO)c2c(-c3ccc4nccn4c3)c[nH]c2n1)C(F)(F)F] | 2178 |
| 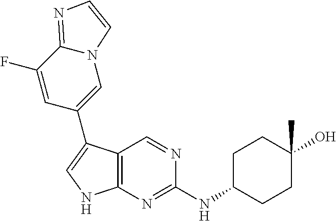 [SMILES: C[C@]1(O)CC[C@@H](Nc2ncc3c(-c4cc(F)c5nccn5c4)c[nH]c3n2)CC1] | 2179 |
| 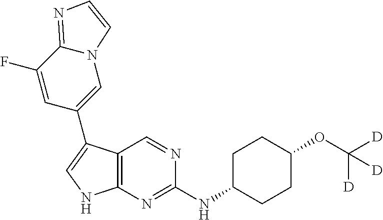 [SMILES: [2H]C([2H])([2H])O[C@H]1CC[C@@H](Nc2ncc3c(-c4cc(F)c5nccn5c4)c[nH]c3n2)CC1] | 2180 |
| 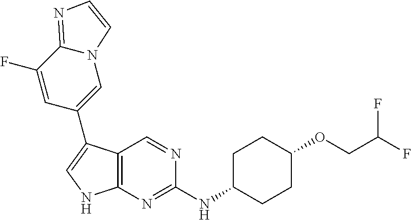 [SMILES: Fc1cc(-c2c[nH]c3nc(N[C@H]4CC[C@@H](OCC(F)F)CC4)ncc23)cn2ccnc12] | 2181 |
| 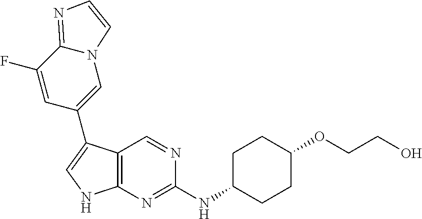 [SMILES: OCCO[C@H]1CC[C@@H](Nc2ncc3c(-c4cc(F)c5nccn5c4)c[nH]c3n2)CC1] | 2182 |
| 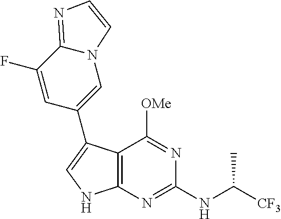 [SMILES: COc1nc(N[C@H](C)C(F)(F)F)nc2[nH]cc(-c3cc(F)c4nccn4c3)c12] | 2183 |
| 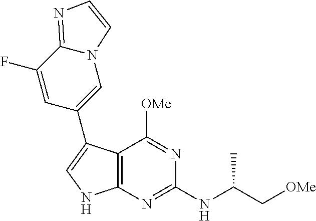 [SMILES: COC[C@@H](C)Nc1nc(OC)c2c(-c3cc(F)c4nccn4c3)c[nH]c2n1] | 2184 |
| 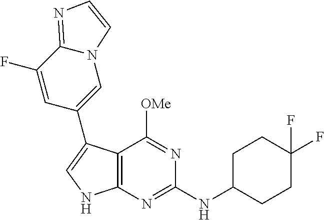 [SMILES: COc1nc(NC2CCC(F)(F)CC2)nc2[nH]cc(-c3cc(F)c4nccn4c3)c12] | 2185 |
| 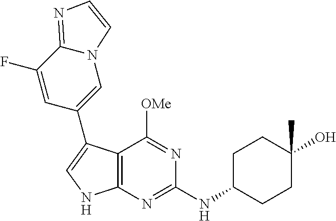 [SMILES: COc1nc(N[C@H]2CC[C@@](C)(O)CC2)nc2[nH]cc(-c3cc(F)c4nccn4c3)c12] | 2186 |
| 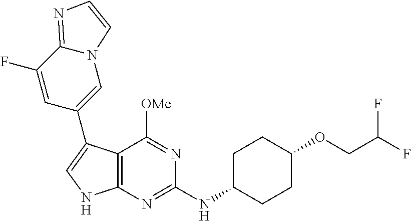 [SMILES: COc1nc(N[C@H]2CC[C@@H](OCC(F)F)CC2)nc2[nH]cc(-c3cc(F)c4nccn4c3)c12] | 2187 |
| 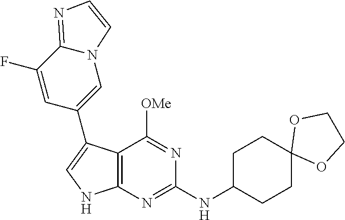 [SMILES: COc1nc(NC2CCC3(CC2)OCCO3)nc2[nH]cc(-c3cc(F)c4nccn4c3)c12] | 2188 |
| 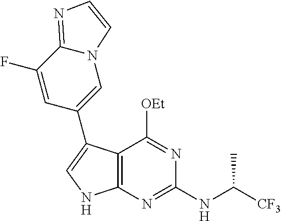 [SMILES: CCOc1nc(N[C@H](C)C(F)(F)F)nc2[nH]cc(-c3cc(F)c4nccn4c3)c12] | 2189 |
| 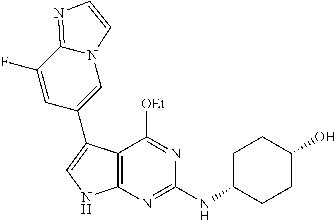 [SMILES: CCOc1nc(N[C@H]2CC[C@@H](O)CC2)nc2[nH]cc(-c3cc(F)c4nccn4c3)c12] | 2190 |
| 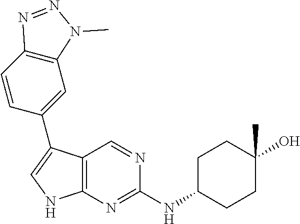 [SMILES: Cn1nnc2ccc(-c3c[nH]c4nc(N[C@H]5CC[C@@](C)(O)CC5)ncc34)cc21] | 2191 |
| 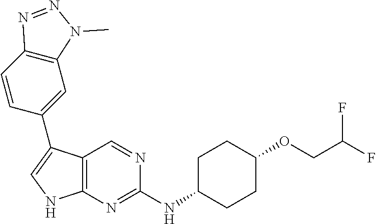 [SMILES: Cn1nnc2ccc(-c3c[nH]c4nc(N[C@H]5CC[C@@H](OCC(F)F)CC5)ncc34)cc21] | 2192 |
| 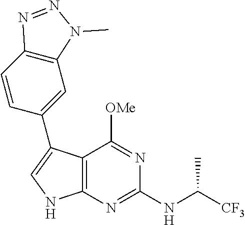 [SMILES: COc1nc(N[C@H](C)C(F)(F)F)nc2[nH]cc(-c3ccc4nnn(C)c4c3)c12] | 2193 |
| 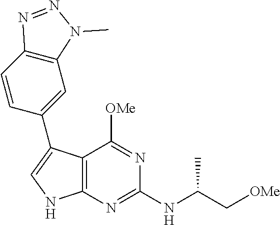 [SMILES: COC[C@@H](C)Nc1nc(OC)c2c(-c3ccc4nnn(C)c4c3)c[nH]c2n1] | 2194 |
| 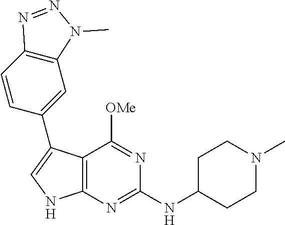 [SMILES: COc1nc(NC2CCN(C)CC2)nc2[nH]cc(-c3ccc4nnn(C)c4c3)c12] | 2195 |
| 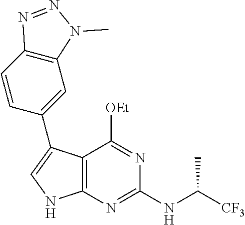 [SMILES: CCOc1nc(N[C@H](C)C(F)(F)F)nc2[nH]cc(-c3ccc4nnn(C)c4c3)c12] | 2196 |
| 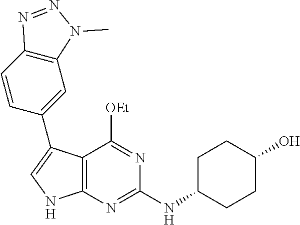 [SMILES: CCOc1nc(N[C@H]2CC[C@@H](O)CC2)nc2[nH]cc(-c3ccc4nnn(C)c4c3)c12] | 2197 |
| 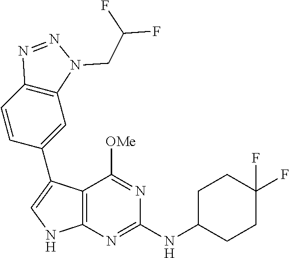 [SMILES: COc1nc(NC2CCC(F)(F)CC2)nc2[nH]cc(-c3ccc4nnn(CC(F)F)c4c3)c12] | 2198 |
| 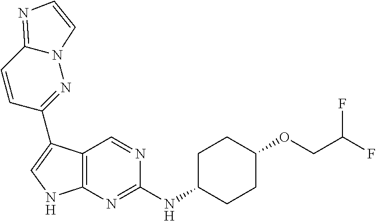 [SMILES: FC(F)CO[C@H]1CC[C@@H](Nc2ncc3c(-c4ccc5nccn5n4)c[nH]c3n2)CC1] | 2199 |
| 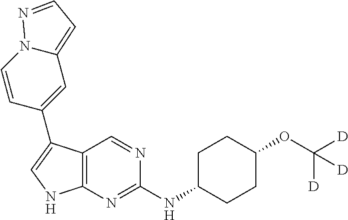 [SMILES: [2H]C([2H])([2H])O[C@H]1CC[C@@H](Nc2ncc3c(-c4ccn5nccc5c4)c[nH]c3n2)CC1] | 2200 |
| 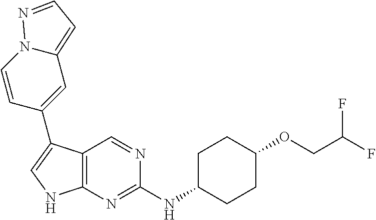 [SMILES: FC(F)CO[C@H]1CC[C@@H](Nc2ncc3c(-c4ccn5nccc5c4)c[nH]c3n2)CC1] | 2201 |
| 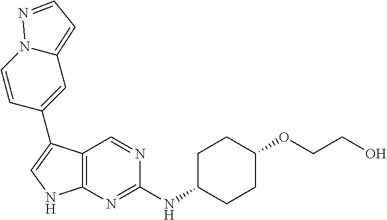 [SMILES: OCCO[C@H]1CC[C@@H](Nc2ncc3c(-c4ccn5nccc5c4)c[nH]c3n2)CC1] | 2202 |
| 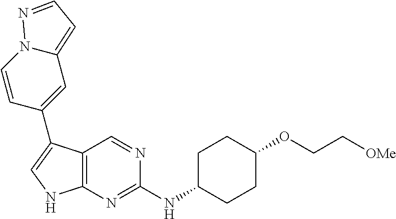 [SMILES: COCCO[C@H]1CC[C@@H](Nc2ncc3c(-c4ccn5nccc5c4)c[nH]c3n2)CC1] | 2203 |
| 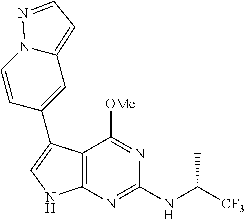 [SMILES: COc1nc(N[C@H](C)C(F)(F)F)nc2[nH]cc(-c3ccn4nccc4c3)c12] | 2204 |
| 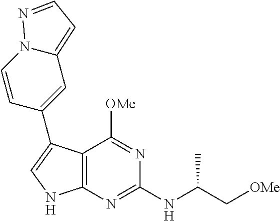 [SMILES: COC[C@@H](C)Nc1nc(OC)c2c(-c3ccn4nccc4c3)c[nH]c2n1] | 2205 |
| 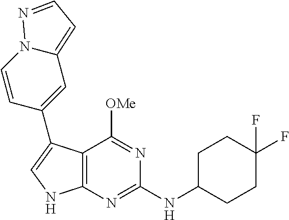 [SMILES: COc1nc(NC2CCC(F)(F)CC2)nc2[nH]cc(-c3ccn4nccc4c3)c12] | 2206 |
| 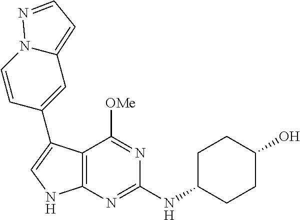 [SMILES: COc1nc(N[C@H]2CC[C@@H](O)CC2)nc2[nH]cc(-c3ccn4nccc4c3)c12] | 2207 |
| 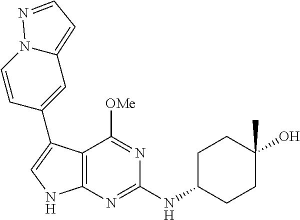 [SMILES: COc1nc(N[C@H]2CC[C@@](C)(O)CC2)nc2[nH]cc(-c3ccn4nccc4c3)c12] | 2208 |
| 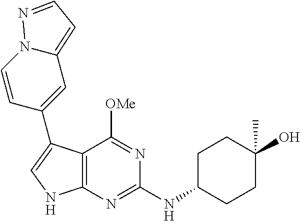 [SMILES: COc1nc(N[C@H]2CC[C@](C)(O)CC2)nc2[nH]cc(-c3ccn4nccc4c3)c12] | 2209 |
| 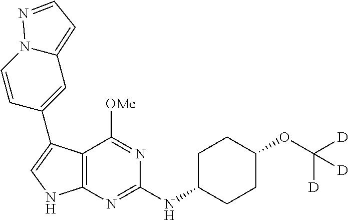 [SMILES: [2H]C([2H])([2H])O[C@H]1CC[C@@H](Nc2nc(OC)c3c(-c4ccn5nccc5c4)c[nH]c3n2)CC1] | 2210 |
| 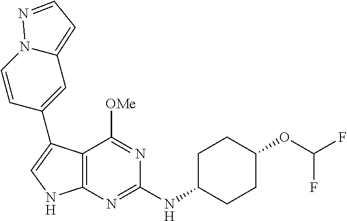 [SMILES: COc1nc(N[C@H]2CC[C@@H](OC(F)F)CC2)nc2[nH]cc(-c3ccn4nccc4c3)c12] | 2211 |
| 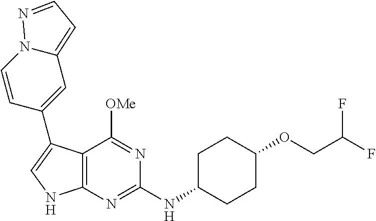 [SMILES: COc1nc(N[C@H]2CC[C@@H](OCC(F)F)CC2)nc2[nH]cc(-c3ccn4nccc4c3)c12] | 2212 |
| 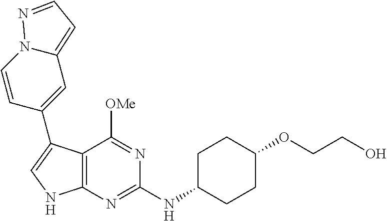 [SMILES: COc1nc(N[C@H]2CC[C@@H](OCCO)CC2)nc2[nH]cc(-c3ccn4nccc4c3)c12] | 2213 |
| 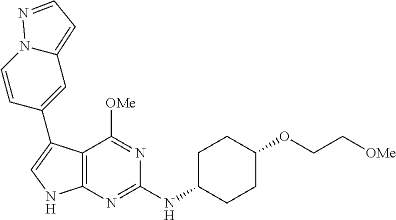 [SMILES: COCCO[C@H]1CC[C@@H](Nc2nc(OC)c3c(-c4ccn5nccc5c4)c[nH]c3n2)CC1] | 2214 |
| 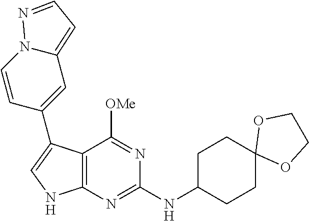 [SMILES: COc1nc(NC2CCC3(CC2)OCCO3)nc2[nH]cc(-c3ccn4nccc4c3)c12] | 2215 |
| 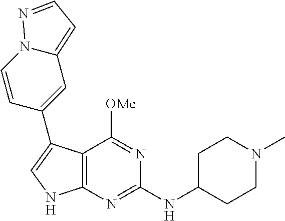 [SMILES: COc1nc(NC2CCN(C)CC2)nc2[nH]cc(-c3ccn4nccc4c3)c12] | 2216 |
| 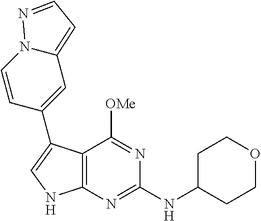 [SMILES: COc1nc(NC2CCOCC2)nc2[nH]cc(-c3ccn4nccc4c3)c12] | 2217 |
| 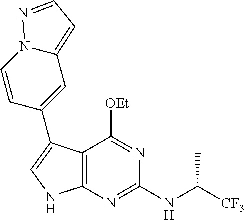 [SMILES: CCOc1nc(N[C@H](C)C(F)(F)F)nc2[nH]cc(-c3ccn4nccc4c3)c12] | 2218 |
| 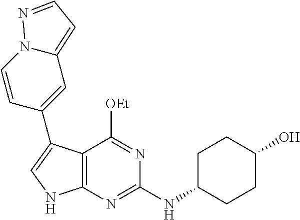 [SMILES: CCOc1nc(N[C@H]2CC[C@@H](O)CC2)nc2[nH]cc(-c3ccn4nccc4c3)c12] | 2219 |
| 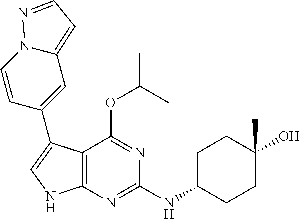 [SMILES: CC(C)Oc1nc(N[C@H]2CC[C@@](C)(O)CC2)nc2[nH]cc(-c3ccn4nccc4c3)c12] | 2220 |
| 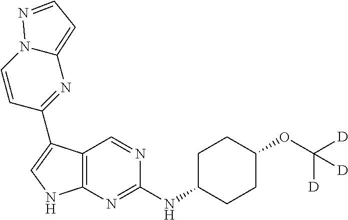 [SMILES: [2H]C([2H])([2H])O[C@H]1CC[C@@H](Nc2ncc3c(-c4ccn5nccc5n4)c[nH]c3n2)CC1] | 2221 |
| 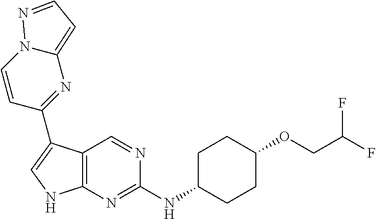 [SMILES: FC(F)CO[C@H]1CC[C@@H](Nc2ncc3c(-c4ccn5nccc5n4)c[nH]c3n2)CC1] | 2222 |
| 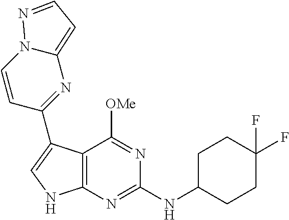 [SMILES: COc1nc(NC2CCC(F)(F)CC2)nc2[nH]cc(-c3ccn4nccc4n3)c12] | 2223 |
| 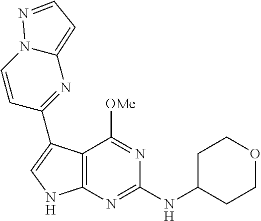 [SMILES: COc1nc(NC2CCOCC2)nc2[nH]cc(-c3ccn4nccc4n3)c12] | 2224 |
| 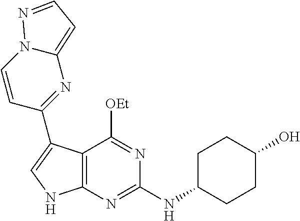 [SMILES: CCOc1nc(N[C@H]2CC[C@@H](O)CC2)nc2[nH]cc(-c3ccn4nccc4n3)c12] | 2225 |
| 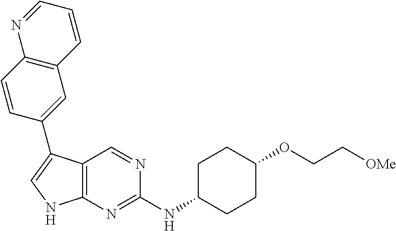 [SMILES: COCCO[C@H]1CC[C@@H](Nc2ncc3c(-c4ccc5ncccc5c4)c[nH]c3n2)CC1] | 2226 |
| 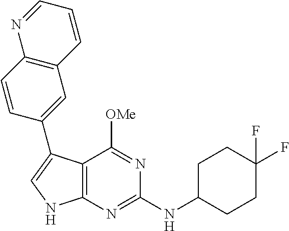 [SMILES: COc1nc(NC2CCC(F)(F)CC2)nc2[nH]cc(-c3ccc4ncccc4c3)c12] | 2227 |
| 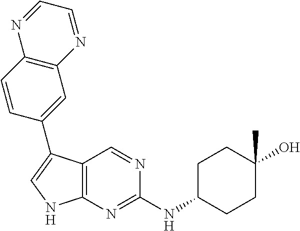 [SMILES: C[C@]1(O)CC[C@@H](Nc2ncc3c(-c4ccc5nccnc5c4)c[nH]c3n2)CC1] | 2228 |
| 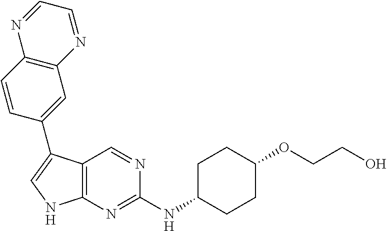 [SMILES: OCCO[C@H]1CC[C@@H](Nc2ncc3c(-c4ccc5nccnc5c4)c[nH]c3n2)CC1] | 2229 |
| 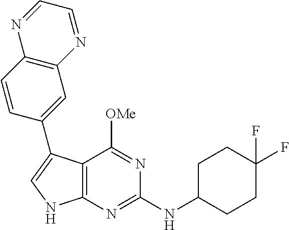 [SMILES: COc1nc(NC2CCC(F)(F)CC2)nc2[nH]cc(-c3ccc4nccnc4c3)c12] | 2230 |
| 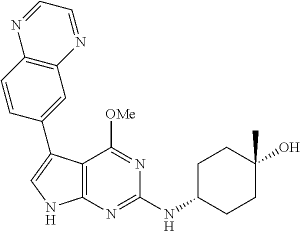 [SMILES: COc1nc(N[C@H]2CC[C@@](C)(O)CC2)nc2[nH]cc(-c3ccc4nccnc4c3)c12] | 2231 |
| 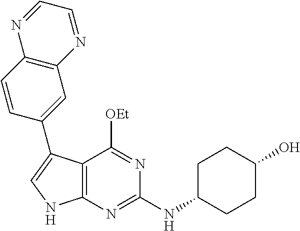 [SMILES: CCOc1nc(N[C@H]2CC[C@@H](O)CC2)nc2[nH]cc(-c3ccc4nccnc4c3)c12] | 2232 |
| 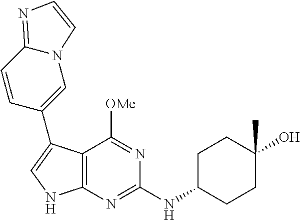 [SMILES: COc1nc(N[C@H]2CC[C@@](C)(O)CC2)nc2[nH]cc(-c3ccc4nccn4c3)c12] | 2233 |
| 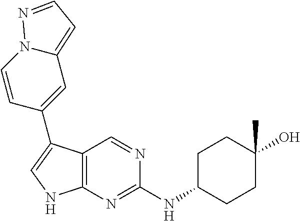 [SMILES: C[C@]1(O)CC[C@@H](Nc2ncc3c(-c4ccn5nccc5c4)c[nH]c3n2)CC1] | 2234 |
| 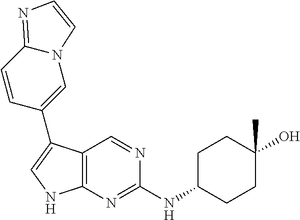 [SMILES: C[C@]1(O)CC[C@@H](Nc2ncc3c(-c4ccc5nccn5c4)c[nH]c3n2)CC1] | 2235 |
| 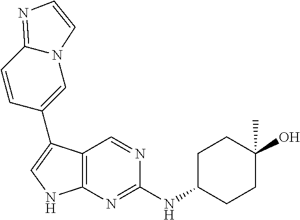 [SMILES: C[C@]1(O)CC[C@H](Nc2ncc3c(-c4ccc5nccn5c4)c[nH]c3n2)CC1] | 2236 |
| 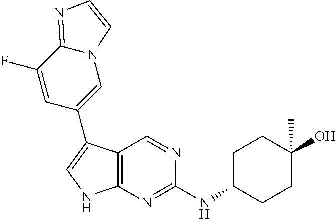 [SMILES: C[C@]1(O)CC[C@H](Nc2ncc3c(-c4cc(F)c5nccn5c4)c[nH]c3n2)CC1] | 2237 |
| 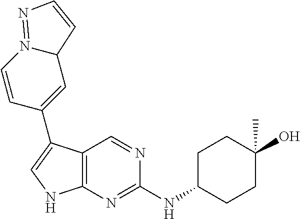 | 2238 |
| 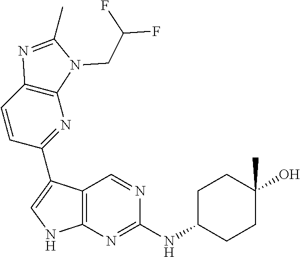 [SMILES: Cc1nc2ccc(-c3c[nH]c4nc(N[C@H]5CC[C@@](C)(O)CC5)ncc34)nc2n1CC(F)F] | 2239 |
| 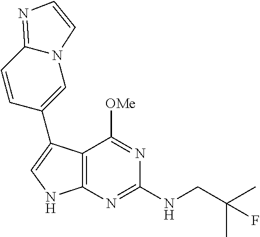 [SMILES: COc1nc(NCC(C)(C)F)nc2[nH]cc(-c3ccc4nccn4c3)c12] | 2240 |
| 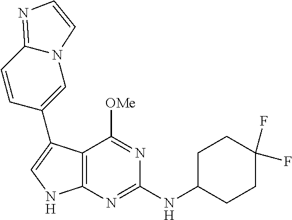 [SMILES: COc1nc(NC2CCC(F)(F)CC2)nc2[nH]cc(-c3ccc4nccn4c3)c12] | 2241 |
| 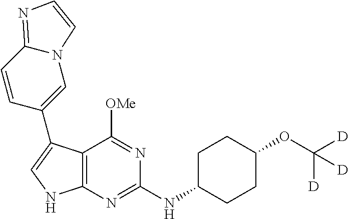 [SMILES: [2H]C([2H])([2H])O[C@H]1CC[C@@H](Nc2nc(OC)c3c(-c4ccc5nccn5c4)c[nH]c3n2)CC1] | 2242 |
| 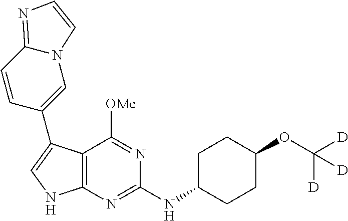 [SMILES: [2H]C([2H])([2H])O[C@H]1CC[C@H](Nc2nc(OC)c3c(-c4ccc5nccn5c4)c[nH]c3n2)CC1] | 2243 |
| 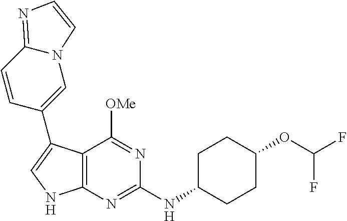 [SMILES: COc1nc(N[C@H]2CC[C@@H](OC(F)F)CC2)nc2[nH]cc(-c3ccc4nccn4c3)c12] | 2244 |
| 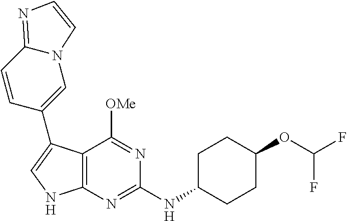 [SMILES: COc1nc(N[C@H]2CC[C@H](OC(F)F)CC2)nc2[nH]cc(-c3ccc4nccn4c3)c12] | 2245 |
| 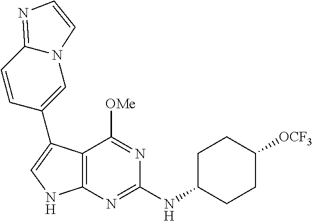 [SMILES: COc1nc(N[C@H]2CC[C@@H](OC(F)(F)F)CC2)nc2[nH]cc(-c3ccc4nccn4c3)c12] | 2246 |
| 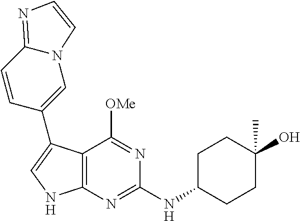 [SMILES: COc1nc(N[C@H]2CC[C@](C)(O)CC2)nc2[nH]cc(-c3ccc4nccn4c3)c12] | 2247 |
| 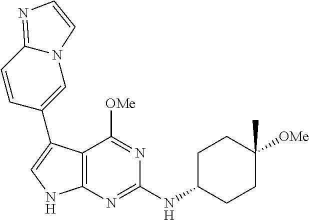 [SMILES: COc1nc(N[C@H]2CC[C@@](C)(OC)CC2)nc2[nH]cc(-c3ccc4nccn4c3)c12] | 2248 |
| 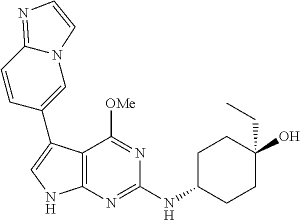 [SMILES: CC[C@]1(O)CC[C@H](Nc2nc(OC)c3c(-c4ccc5nccn5c4)c[nH]c3n2)CC1] | 2249 |
| 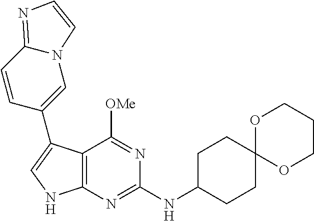 [SMILES: COc1nc(NC2CCC3(CC2)OCCCO3)nc2[nH]cc(-c3ccc4nccn4c3)c12] | 2250 |
| 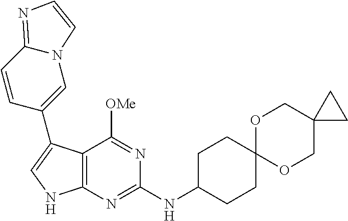 [SMILES: COc1nc(NC2CCC3(CC2)OCC2(CC2)CO3)nc2[nH]cc(-c3ccc4nccn4c3)c12] | 2251 |
| 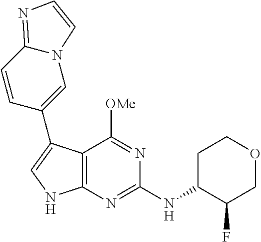 | 2252 |
| 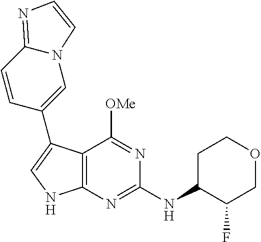 | 2253 |
| 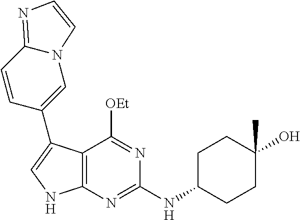 [SMILES: CCOc1nc(N[C@H]2CC[C@@](C)(O)CC2)nc2[nH]cc(-c3ccc4nccn4c3)c12] | 2254 |
| 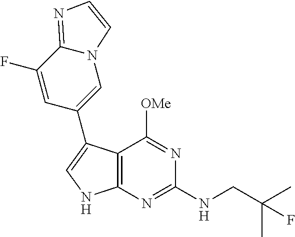 [SMILES: COc1nc(NCC(C)(C)F)nc2[nH]cc(-c3cc(F)c4nccn4c3)c12] | 2255 |
| 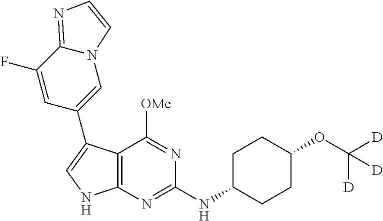 [SMILES: [2H]C([2H])([2H])O[C@H]1CC[C@@H](Nc2nc(OC)c3c(-c4cc(F)c5nccn5c4)c[nH]c3n2)CC1] | 2256 |
| 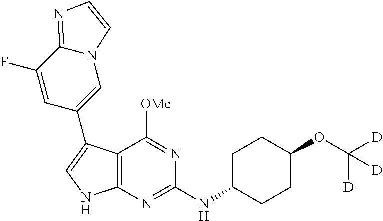 [SMILES: [2H]C([2H])([2H])O[C@H]1CC[C@H](Nc2nc(OC)c3c(-c4cc(F)c5nccn5c4)c[nH]c3n2)CC1] | 2257 |
| 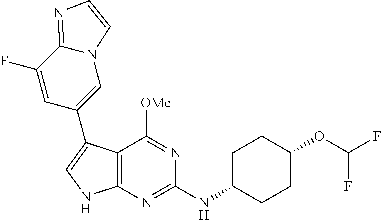 [SMILES: COc1nc(N[C@H]2CC[C@@H](OC(F)F)CC2)nc2[nH]cc(-c3cc(F)c4nccn4c3)c12] | 2258 |
| 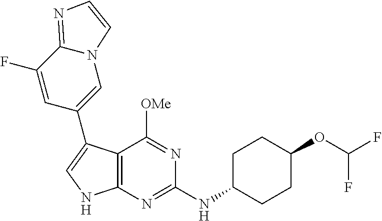 [SMILES: COc1nc(N[C@H]2CC[C@H](OC(F)F)CC2)nc2[nH]cc(-c3cc(F)c4nccn4c3)c12] | 2259 |
| 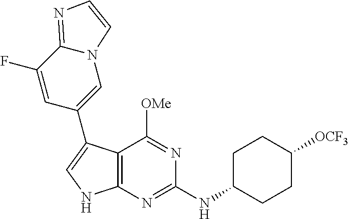 [SMILES: COc1nc(N[C@H]2CC[C@@H](OC(F)(F)F)CC2)nc2[nH]cc(-c3cc(F)c4nccn4c3)c12] | 2260 |
| 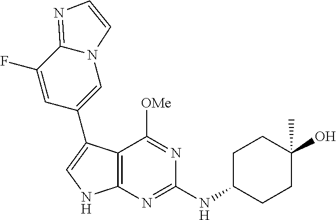 [SMILES: COc1nc(N[C@H]2CC[C@](C)(O)CC2)nc2[nH]cc(-c3cc(F)c4nccn4c3)c12] | 2261 |
| 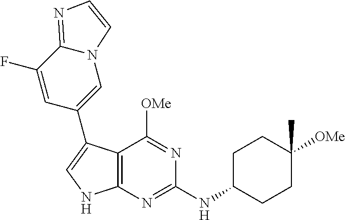 [SMILES: COc1nc(N[C@H]2CC[C@@](C)(OC)CC2)nc2[nH]cc(-c3cc(F)c4nccn4c3)c12] | 2262 |
| 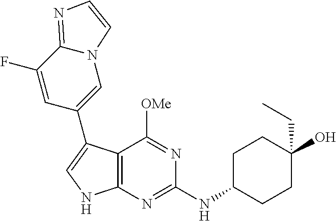 [SMILES: CC[C@]1(O)CC[C@H](Nc2nc(OC)c3c(-c4cc(F)c5nccn5c4)c[nH]c3n2)CC1] | 2263 |
| 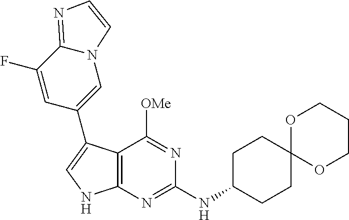 | 2264 |
| 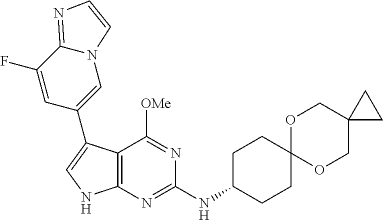 | 2265 |
| 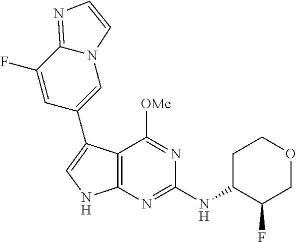 | 2266 |
| 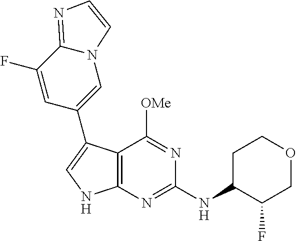 | 2267 |
| 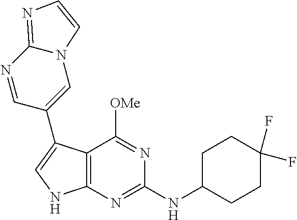 [SMILES: COc1nc(NC2CCC(F)(F)CC2)nc2[nH]cc(-c3cnc4nccn4c3)c12] | 2268 |
| 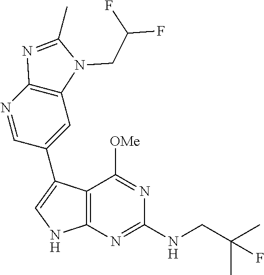 [SMILES: COc1nc(NCC(C)(C)F)nc2[nH]cc(-c3cnc4nc(C)n(CC(F)F)c4c3)c12] | 2269 |
| 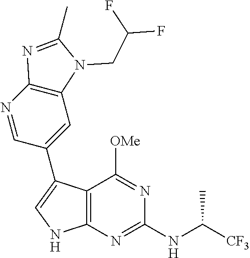 [SMILES: COc1nc(N[C@H](C)C(F)(F)F)nc2[nH]cc(-c3cnc4nc(C)n(CC(F)F)c4c3)c12] | 2270 |
| 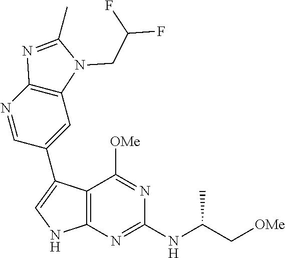 [SMILES: COC[C@@H](C)Nc1nc(OC)c2c(-c3cnc4nc(C)n(CC(F)F)c4c3)c[nH]c2n1] | 2271 |
| 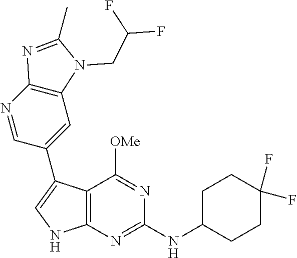 [SMILES: COc1nc(NC2CCC(F)(F)CC2)nc2[nH]cc(-c3cnc4nc(C)n(CC(F)F)c4c3)c12] | 2272 |
| 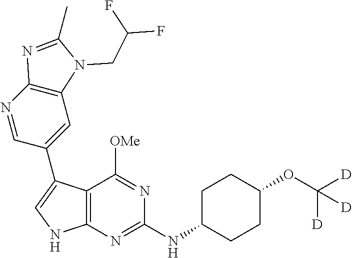 [SMILES: [2H]C([2H])([2H])O[C@H]1CC[C@@H](Nc2nc(OC)c3c(-c4cnc5nc(C)n(CC(F)F)c5c4)c[nH]c3n2)CC1] | 2273 |
| 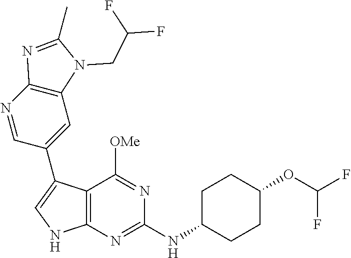 [SMILES: COc1nc(N[C@H]2CC[C@@H](OC(F)F)CC2)nc2[nH]cc(-c3cnc4nc(C)n(CC(F)F)c4c3)c12] | 2274 |
| 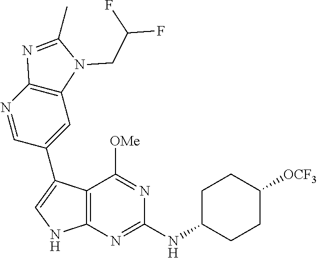 [SMILES: COc1nc(N[C@H]2CC[C@@H](OC(F)(F)F)CC2)nc2[nH]cc(-c3cnc4nc(C)n(CC(F)F)c4c3)c12] | 2275 |
| 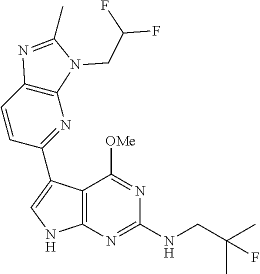 [SMILES: COc1nc(NCC(C)(C)F)nc2[nH]cc(-c3ccc4nc(C)n(CC(F)F)c4n3)c12] | 2276 |
| 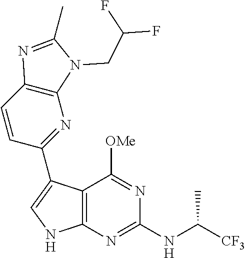 [SMILES: COc1nc(N[C@H](C)C(F)(F)F)nc2[nH]cc(-c3ccc4nc(C)n(CC(F)F)c4n3)c12] | 2277 |
| 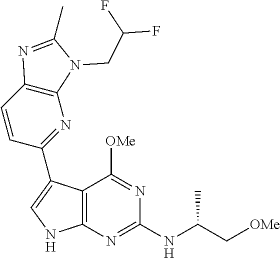 [SMILES: COC[C@@H](C)Nc1nc(OC)c2c(-c3ccc4nc(C)n(CC(F)F)c4n3)c[nH]c2n1] | 2278 |
| 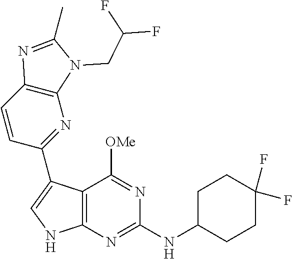 [SMILES: COc1nc(NC2CCC(F)(F)CC2)nc2[nH]cc(-c3ccc4nc(C)n(CC(F)F)c4n3)c12] | 2279 |
| 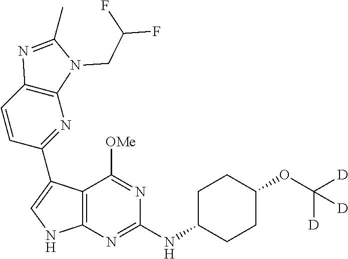 [SMILES: [2H]C([2H])([2H])O[C@H]1CC[C@@H](Nc2nc(OC)c3c(-c4ccc5nc(C)n(CC(F)F)c5n4)c[nH]c3n2)CC1] | 2280 |
| 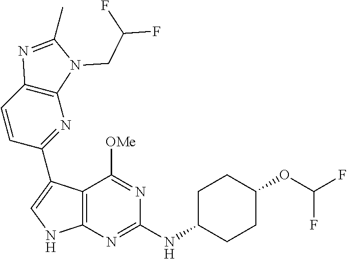 [SMILES: COc1nc(N[C@H]2CC[C@@H](OC(F)F)CC2)nc2[nH]cc(-c3ccc4nc(C)n(CC(F)F)c4n3)c12] | 2281 |
| 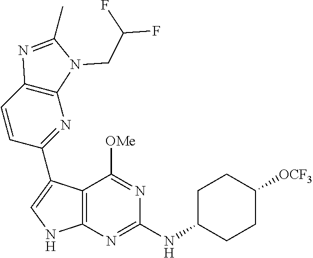 [SMILES: COc1nc(N[C@H]2CC[C@@H](OC(F)(F)F)CC2)nc2[nH]cc(-c3ccc4nc(C)n(CC(F)F)c4n3)c12] | 2282 |
| 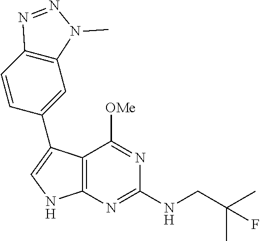 [SMILES: COc1nc(NCC(C)(C)F)nc2[nH]cc(-c3ccc4nnn(C)c4c3)c12] | 2283 |
| 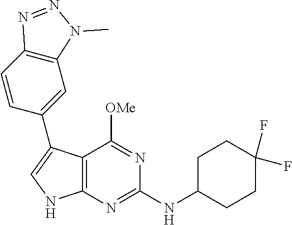 [SMILES: COc1nc(NC2CCC(F)(F)CC2)nc2[nH]cc(-c3ccc4nnn(C)c4c3)c12] | 2284 |
| 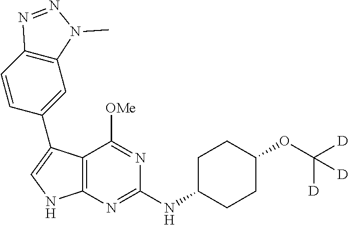 [SMILES: [2H]C([2H])([2H])O[C@H]1CC[C@@H](Nc2nc(OC)c3c(-c4ccc5nnn(C)c5c4)c[nH]c3n2)CC1] | 2285 |
| 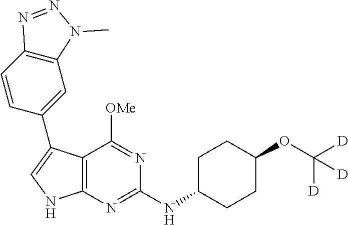 [SMILES: [2H]C([2H])([2H])O[C@H]1CC[C@H](Nc2nc(OC)c3c(-c4ccc5nnn(C)c5c4)c[nH]c3n2)CC1] | 2286 |
| 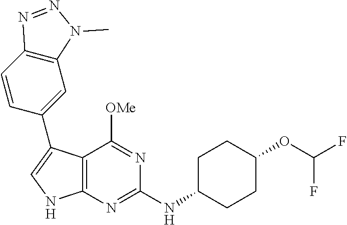 [SMILES: COc1nc(N[C@H]2CC[C@@H](OC(F)F)CC2)nc2[nH]cc(-c3ccc4nnn(C)c4c3)c12] | 2287 |
| 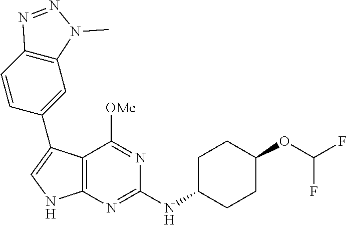 [SMILES: COc1nc(N[C@H]2CC[C@H](OC(F)F)CC2)nc2[nH]cc(-c3ccc4nnn(C)c4c3)c12] | 2288 |
| 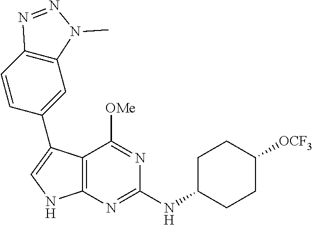 [SMILES: COc1nc(N[C@H]2CC[C@@H](OC(F)(F)F)CC2)nc2[nH]cc(-c3ccc4nnn(C)c4c3)c12] | 2289 |
| 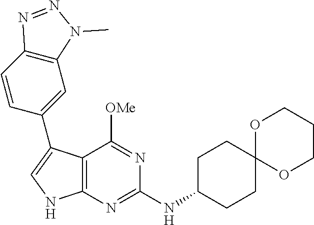 | 2290 |
| 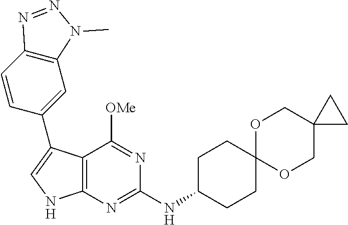 | 2291 |
| 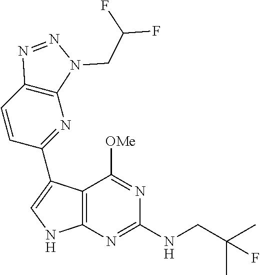 [SMILES: COc1nc(NCC(C)(C)F)nc2[nH]cc(-c3ccc4nnn(CC(F)F)c4n3)c12] | 2292 |
| 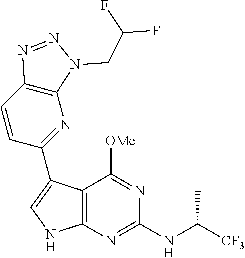 [SMILES: COc1nc(N[C@H](C)C(F)(F)F)nc2[nH]cc(-c3ccc4nnn(CC(F)F)c4n3)c12] | 2293 |
| 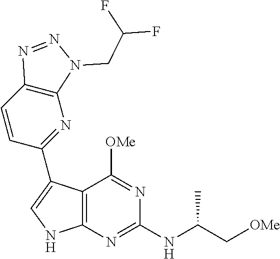 [SMILES: COC[C@@H](C)Nc1nc(OC)c2c(-c3ccc4nnn(CC(F)F)c4n3)c[nH]c2n1] | 2294 |
| 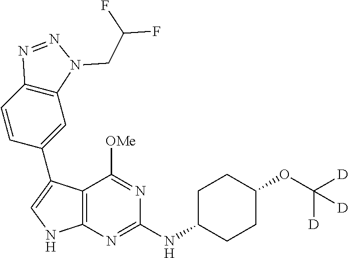 [SMILES: [2H]C([2H])([2H])O[C@H]1CC[C@@H](Nc2nc(OC)c3c(-c4ccc5nnn(CC(F)F)c5c4)c[nH]c3n2)CC1] | 2295 |
| 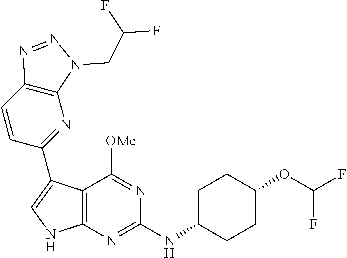 [SMILES: COc1nc(N[C@H]2CC[C@@H](OC(F)F)CC2)nc2[nH]cc(-c3ccc4nnn(CC(F)F)c4n3)c12] | 2296 |
| 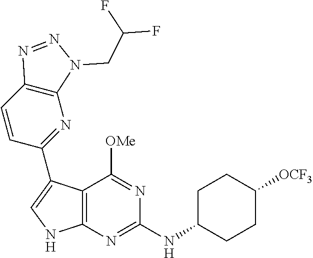 [SMILES: COc1nc(N[C@H]2CC[C@@H](OC(F)(F)F)CC2)nc2[nH]cc(-c3ccc4nnn(CC(F)F)c4n3)c12] | 2297 |
| 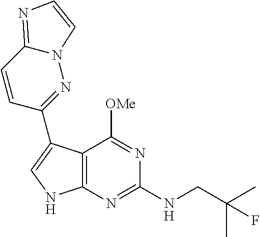 [SMILES: COc1nc(NCC(C)(C)F)nc2[nH]cc(-c3ccc4nccn4n3)c12] | 2298 |
| 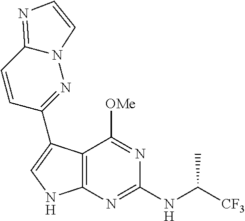 [SMILES: COc1nc(N[C@H](C)C(F)(F)F)nc2[nH]cc(-c3ccc4nccn4n3)c12] | 2299 |
| 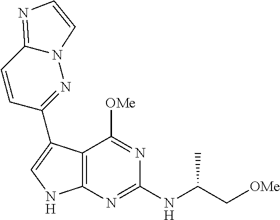 [SMILES: COC[C@@H](C)Nc1nc(OC)c2c(-c3ccc4nccn4n3)c[nH]c2n1] | 2300 |
| 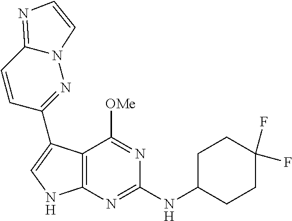 [SMILES: COc1nc(NC2CCC(F)(F)CC2)nc2[nH]cc(-c3ccc4nccn4n3)c12] | 2301 |
| 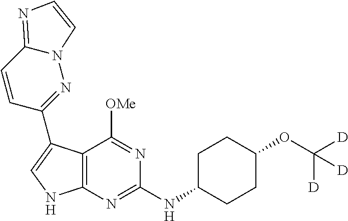 [SMILES: [2H]C([2H])([2H])O[C@H]1CC[C@@H](Nc2nc(OC)c3c(-c4ccc5nccn5n4)c[nH]c3n2)CC1] | 2302 |
| 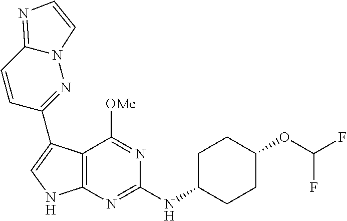 [SMILES: COc1nc(N[C@H]2CC[C@@H](OC(F)F)CC2)nc2[nH]cc(-c3ccc4nccn4n3)c12] | 2303 |
| 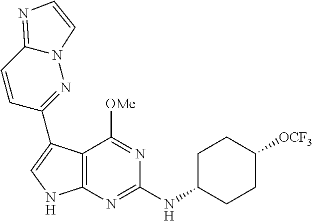 [SMILES: COc1nc(N[C@H]2CC[C@@H](OC(F)(F)F)CC2)nc2[nH]cc(-c3ccc4nccn4n3)c12] | 2304 |
| 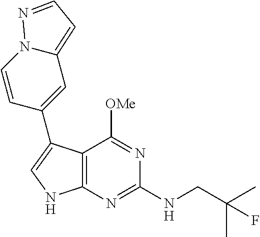 [SMILES: COc1nc(NCC(C)(C)F)nc2[nH]cc(-c3ccn4nccc4c3)c12] | 2305 |
| 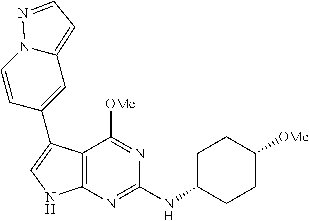 [SMILES: COc1nc(N[C@H]2CC[C@@H](OC)CC2)nc2[nH]cc(-c3ccn4nccc4c3)c12] | 2306 |
| 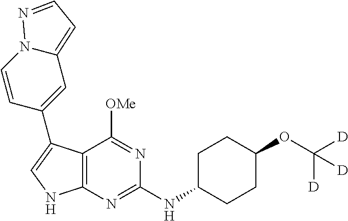 [SMILES: [2H]C([2H])([2H])O[C@H]1CC[C@H](Nc2nc(OC)c3c(-c4ccn5nccc5c4)c[nH]c3n2)CC1] | 2307 |
| 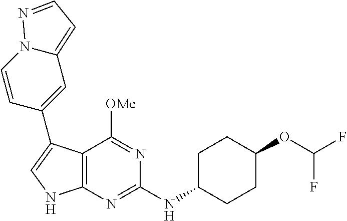 [SMILES: COc1nc(N[C@H]2CC[C@H](OC(F)F)CC2)nc2[nH]cc(-c3ccn4nccc4c3)c12] | 2308 |
| 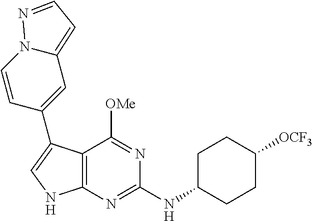 [SMILES: COc1nc(N[C@H]2CC[C@@H](OC(F)(F)F)CC2)nc2[nH]cc(-c3ccn4nccc4c3)c12] | 2309 |
| 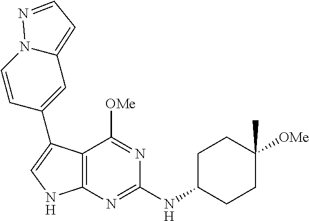 [SMILES: COc1nc(N[C@H]2CC[C@@](C)(OC)CC2)nc2[nH]cc(-c3ccn4nccc4c3)c12] | 2310 |
| 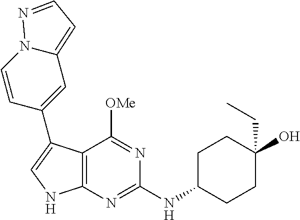 [SMILES: CC[C@]1(O)CC[C@H](Nc2nc(OC)c3c(-c4ccn5nccc5c4)c[nH]c3n2)CC1] | 2311 |
| 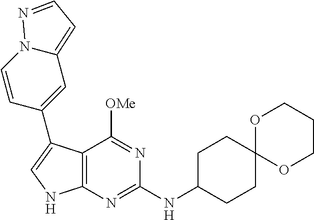 [SMILES: COc1nc(NC2CCC3(CC2)OCCCO3)nc2[nH]cc(-c3ccn4nccc4c3)c12] | 2312 |
| 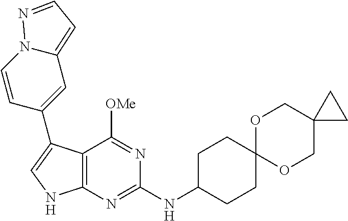 [SMILES: COc1nc(NC2CCC3(CC2)OCC2(CC2)CO3)nc2[nH]cc(-c3ccn4nccc4c3)c12] | 2313 |
| 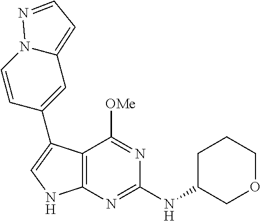 | 2314 |
| 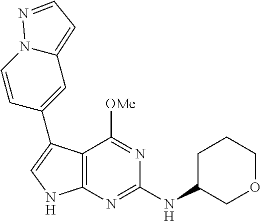 | 2315 |
| 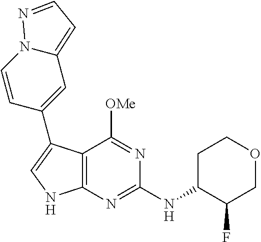 | 2316 |
| 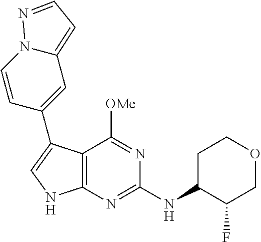 | 2317 |
| 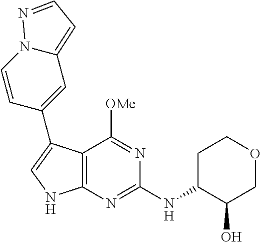 | 2318 |
| 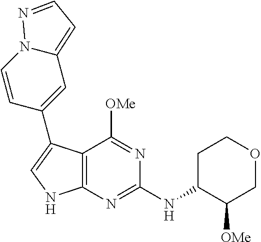 | 2319 |
| 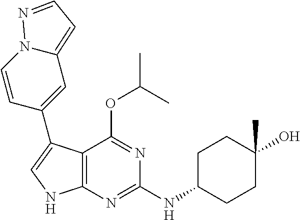 [SMILES: CC(C)Oc1nc(N[C@H]2CC[C@@](C)(O)CC2)nc2[nH]cc(-c3ccn4nccc4c3)c12] | 2320 |
| 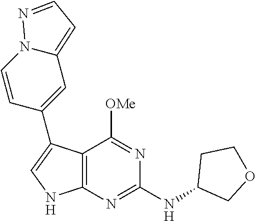 [SMILES: COc1nc(N[C@@H]2CCOC2)nc2[nH]cc(-c3ccn4nccc4c3)c12] | 2321 |
| 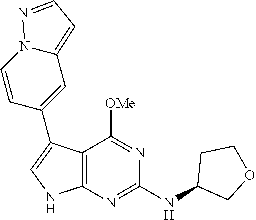 [SMILES: COc1nc(N[C@H]2CCOC2)nc2[nH]cc(-c3ccn4nccc4c3)c12] | 2322 |
| 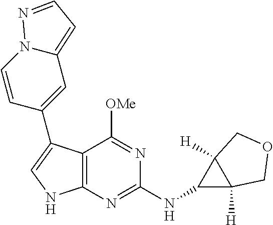 | 2323 |
| 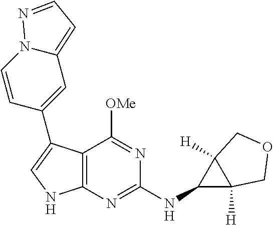 [SMILES: COc1nc(N[C@@H]2[C@@H]3COC[C@@H]32)nc2[nH]cc(-c3ccn4nccc4c3)c12] | 2324 |
| 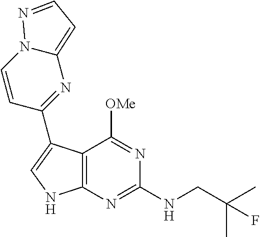 [SMILES: COc1nc(NCC(C)(C)F)nc2[nH]cc(-c3ccn4nccc4n3)c12] | 2325 |
| 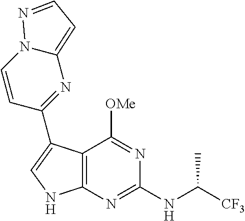 [SMILES: COc1nc(N[C@H](C)C(F)(F)F)nc2[nH]cc(-c3ccn4nccc4n3)c12] | 2326 |
| 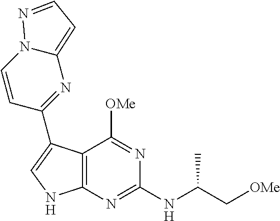 [SMILES: COC[C@@H](C)Nc1nc(OC)c2c(-c3ccn4nccc4n3)c[nH]c2n1] | 2327 |
| 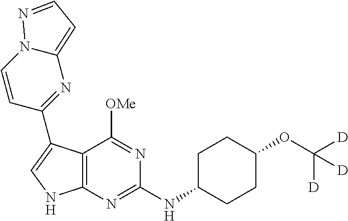 [SMILES: [2H]C([2H])([2H])O[C@H]1CC[C@@H](Nc2nc(OC)c3c(-c4ccn5nccc5n4)c[nH]c3n2)CC1] | 2328 |
| 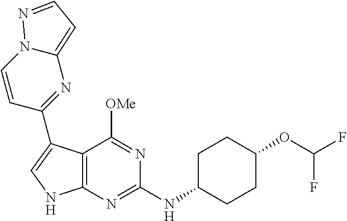 [SMILES: COc1nc(N[C@H]2CC[C@@H](OC(F)F)CC2)nc2[nH]cc(-c3ccn4nccc4n3)c12] | 2329 |
| 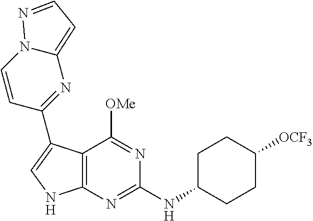 [SMILES: COc1nc(N[C@H]2CC[C@@H](OC(F)(F)F)CC2)nc2[nH]cc(-c3ccn4nccc4n3)c12] | 2330 |
| 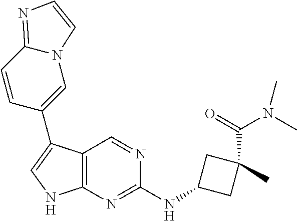 [SMILES: CN(C)C(=O)[C@]1(C)C[C@H](Nc2ncc3c(-c4ccc5nccn5c4)c[nH]c3n2)C1] | 2331 |
| 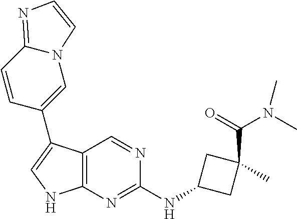 [SMILES: CN(C)C(=O)[C@]1(C)C[C@@H](Nc2ncc3c(-c4ccc5nccn5c4)c[nH]c3n2)C1] | 2332 |
| 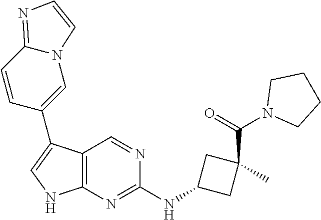 [SMILES: C[C@]1(C(=O)N2CCCC2)C[C@H](Nc2ncc3c(-c4ccc5nccn5c4)c[nH]c3n2)C1] | 2333 |
| 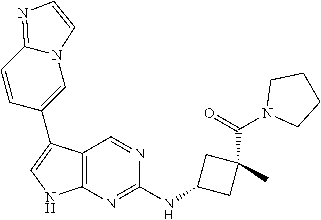 [SMILES: C[C@]1(C(=O)N2CCCC2)C[C@@H](Nc2ncc3c(-c4ccc5nccn5c4)c[nH]c3n2)C1] | 2334 |
| 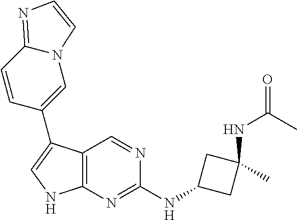 [SMILES: CC(=O)N[C@]1(C)C[C@@H](Nc2ncc3c(-c4ccc5nccn5c4)c[nH]c3n2)C1] | 2335 |
| 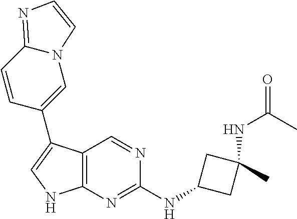 [SMILES: CC(=O)N[C@]1(C)C[C@H](Nc2ncc3c(-c4ccc5nccn5c4)c[nH]c3n2)C1] | 2336 |
| 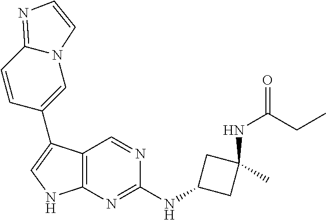 [SMILES: CCC(=O)N[C@]1(C)C[C@@H](Nc2ncc3c(-c4ccc5nccn5c4)c[nH]c3n2)C1] | 2337 |
| 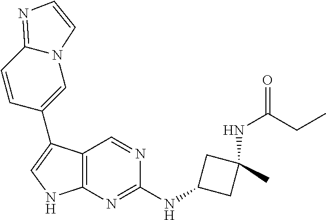 [SMILES: CCC(=O)N[C@]1(C)C[C@H](Nc2ncc3c(-c4ccc5nccn5c4)c[nH]c3n2)C1] | 2338 |
| 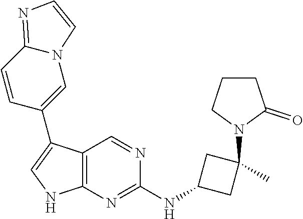 [SMILES: C[C@]1(N2CCCC2=O)C[C@H](Nc2ncc3c(-c4ccc5nccn5c4)c[nH]c3n2)C1] | 2339 |
| 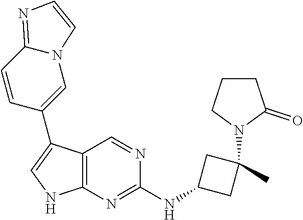 [SMILES: C[C@]1(N2CCCC2=O)C[C@@H](Nc2ncc3c(-c4ccc5nccn5c4)c[nH]c3n2)C1] | 2340 |
| 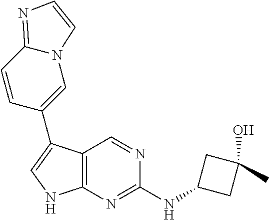 [SMILES: C[C@]1(O)C[C@@H](Nc2ncc3c(-c4ccc5nccn5c4)c[nH]c3n2)C1] | 2341 |
| 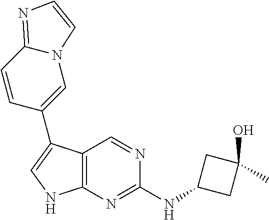 [SMILES: C[C@]1(O)C[C@H](Nc2ncc3c(-c4ccc5nccn5c4)c[nH]c3n2)C1] | 2342 |
| 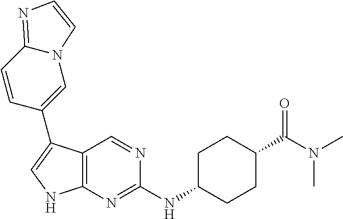 [SMILES: CN(C)C(=O)[C@H]1CC[C@@H](Nc2ncc3c(-c4ccc5nccn5c4)c[nH]c3n2)CC1] | 2343 |
| 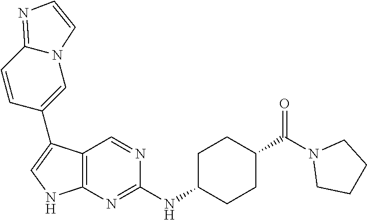 [SMILES: O=C([C@H]1CC[C@@H](Nc2ncc3c(-c4ccc5nccn5c4)c[nH]c3n2)CC1)N1CCCC1] | 2344 |
| 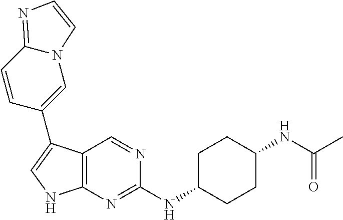 [SMILES: CC(=O)N[C@H]1CC[C@@H](Nc2ncc3c(-c4ccc5nccn5c4)c[nH]c3n2)CC1] | 2345 |
| 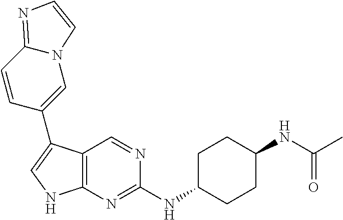 [SMILES: CC(=O)N[C@H]1CC[C@H](Nc2ncc3c(-c4ccc5nccn5c4)c[nH]c3n2)CC1] | 2346 |
| 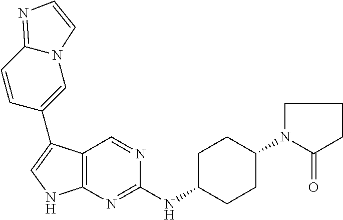 [SMILES: O=C1CCCN1[C@H]1CC[C@@H](Nc2ncc3c(-c4ccc5nccn5c4)c[nH]c3n2)CC1] | 2347 |
| 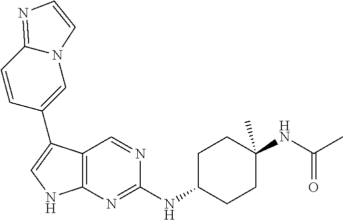 [SMILES: CC(=O)N[C@]1(C)CC[C@@H](Nc2ncc3c(-c4ccc5nccn5c4)c[nH]c3n2)CC1] | 2348 |
| 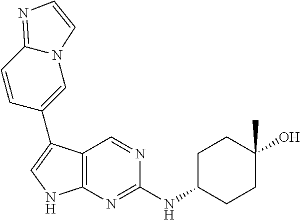 [SMILES: C[C@]1(O)CC[C@@H](Nc2ncc3c(-c4ccc5nccn5c4)c[nH]c3n2)CC1] | 2349 |
| 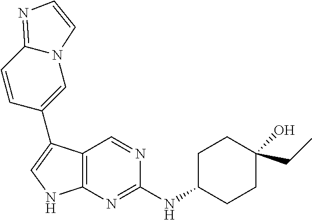 [SMILES: CC[C@]1(O)CC[C@@H](Nc2ncc3c(-c4ccc5nccn5c4)c[nH]c3n2)CC1] | 2350 |
| 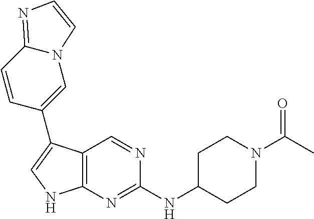 [SMILES: CC(=O)N1CCC(Nc2ncc3c(-c4ccc5nccn5c4)c[nH]c3n2)CC1] | 2351 |
| 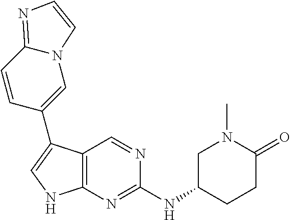 [SMILES: CN1C[C@@H](Nc2ncc3c(-c4ccc5nccn5c4)c[nH]c3n2)CCC1=O] | 2352 |
| 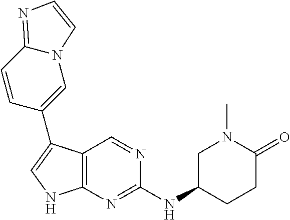 [SMILES: CN1C[C@H](Nc2ncc3c(-c4ccc5nccn5c4)c[nH]c3n2)CCC1=O] | 2353 |
| 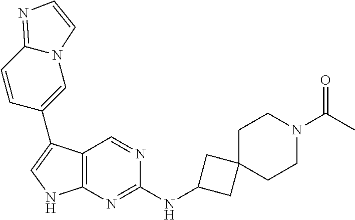 [SMILES: CC(=O)N1CCC2(CC1)CC(Nc1ncc3c(-c4ccc5nccn5c4)c[nH]c3n1)C2] | 2354 |
| 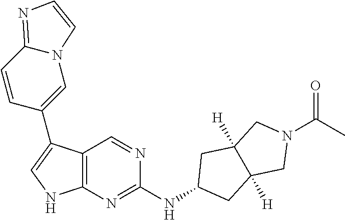 [SMILES: CC(=O)N1C[C@H]2C[C@H](Nc3ncc4c(-c5ccc6nccn6c5)c[nH]c4n3)C[C@H]2C1] | 2355 |
| 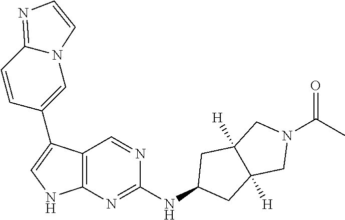 [SMILES: CC(=O)N1C[C@H]2C[C@@H](Nc3ncc4c(-c5ccc6nccn6c5)c[nH]c4n3)C[C@H]2C1] | 2356 |
| 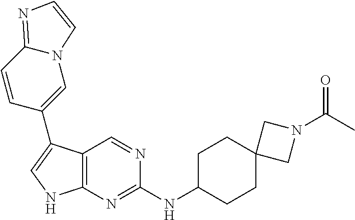 [SMILES: CC(=O)N1CC2(CCC(Nc3ncc4c(-c5ccc6nccn6c5)c[nH]c4n3)CC2)C1] | 2357 |
| 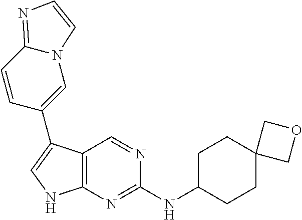 [SMILES: c1cn2cc(-c3c[nH]c4nc(NC5CCC6(CC5)COC6)ncc34)ccc2n1] | 2358 |
| 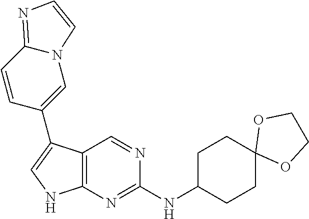 [SMILES: c1cn2cc(-c3c[nH]c4nc(NC5CCC6(CC5)OCCO6)ncc34)ccc2n1] | 2359 |
| 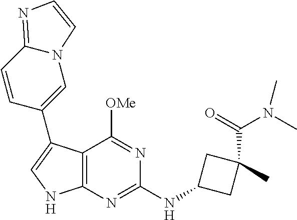 [SMILES: COc1nc(N[C@H]2C[C@@](C)(C(=O)N(C)C)C2)nc2[nH]cc(-c3ccc4nccn4c3)c12] | 2360 |
| 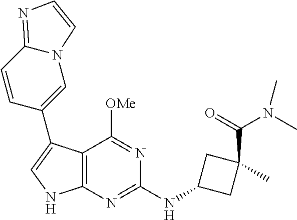 [SMILES: COc1nc(N[C@H]2C[C@](C)(C(=O)N(C)C)C2)nc2[nH]cc(-c3ccc4nccn4c3)c12] | 2361 |
| 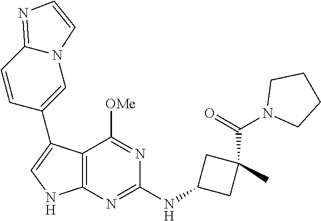 [SMILES: COc1nc(N[C@H]2C[C@@](C)(C(=O)N3CCCC3)C2)nc2[nH]cc(-c3ccc4nccn4c3)c12] | 2362 |
| 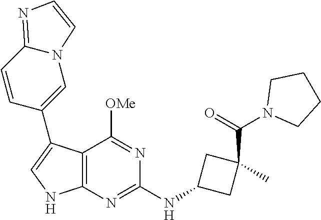 [SMILES: COc1nc(N[C@H]2C[C@](C)(C(=O)N3CCCC3)C2)nc2[nH]cc(-c3ccc4nccn4c3)c12] | 2363 |
| 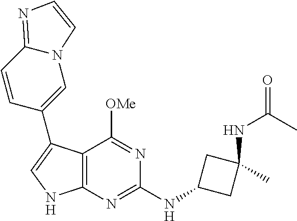 [SMILES: COc1nc(N[C@H]2C[C@](C)(NC(C)=O)C2)nc2[nH]cc(-c3ccc4nccn4c3)c12] | 2364 |
| 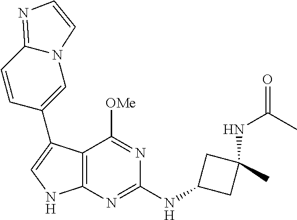 [SMILES: COc1nc(N[C@H]2C[C@@](C)(NC(C)=O)C2)nc2[nH]cc(-c3ccc4nccn4c3)c12] | 2365 |
| 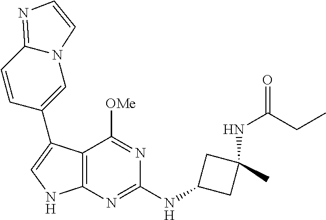 [SMILES: CCC(=O)N[C@]1(C)C[C@H](Nc2nc(OC)c3c(-c4ccc5nccn5c4)c[nH]c3n2)C1] | 2366 |
| 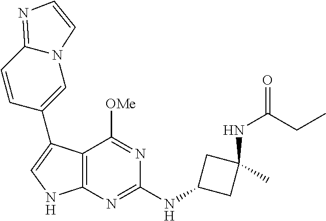 [SMILES: CCC(=O)N[C@]1(C)C[C@@H](Nc2nc(OC)c3c(-c4ccc5nccn5c4)c[nH]c3n2)C1] | 2367 |
| 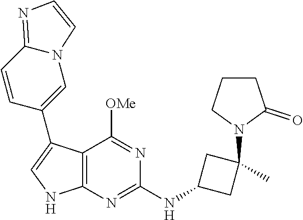 [SMILES: COc1nc(N[C@H]2C[C@](C)(N3CCCC3=O)C2)nc2[nH]cc(-c3ccc4nccn4c3)c12] | 2368 |
| 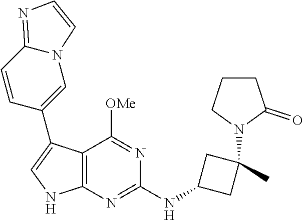 [SMILES: COc1nc(N[C@H]2C[C@@](C)(N3CCCC3=O)C2)nc2[nH]cc(-c3ccc4nccn4c3)c12] | 2369 |
| 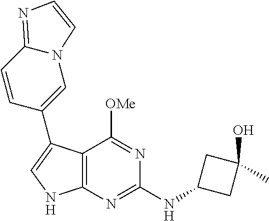 [SMILES: COc1nc(N[C@H]2C[C@](C)(O)C2)nc2[nH]cc(-c3ccc4nccn4c3)c12] | 2370 |
| 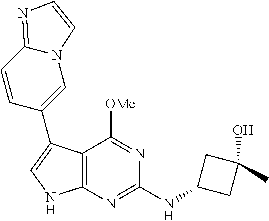 [SMILES: COc1nc(N[C@H]2C[C@@](C)(O)C2)nc2[nH]cc(-c3ccc4nccn4c3)c12] | 2371 |
| 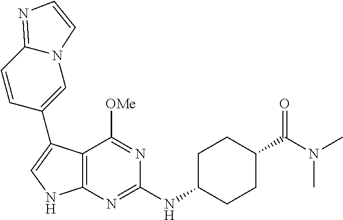 [SMILES: COc1nc(N[C@H]2CC[C@@H](C(=O)N(C)C)CC2)nc2[nH]cc(-c3ccc4nccn4c3)c12] | 2372 |
| 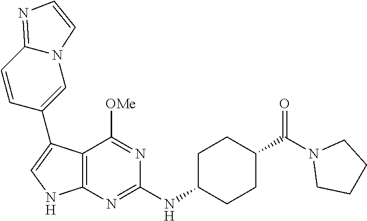 [SMILES: COc1nc(N[C@H]2CC[C@@H](C(=O)N3CCCC3)CC2)nc2[nH]cc(-c3ccc4nccn4c3)c12] | 2373 |
| 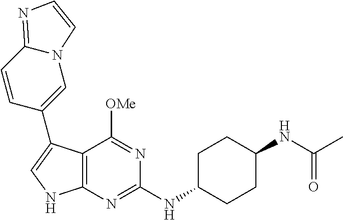 [SMILES: COc1nc(N[C@H]2CC[C@H](NC(C)=O)CC2)nc2[nH]cc(-c3ccc4nccn4c3)c12] | 2374 |
| 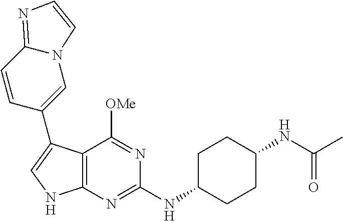 [SMILES: COc1nc(N[C@H]2CC[C@@H](NC(C)=O)CC2)nc2[nH]cc(-c3ccc4nccn4c3)c12] | 2375 |
| 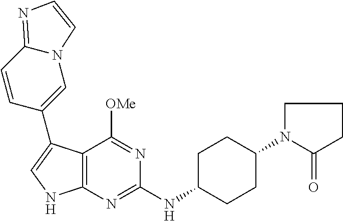 [SMILES: COc1nc(N[C@H]2CC[C@@H](N3CCCC3=O)CC2)nc2[nH]cc(-c3ccc4nccn4c3)c12] | 2376 |
| 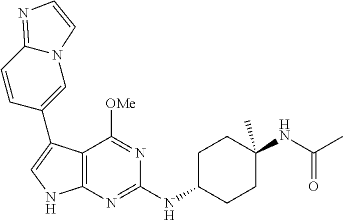 [SMILES: COc1nc(N[C@H]2CC[C@](C)(NC(C)=O)CC2)nc2[nH]cc(-c3ccc4nccn4c3)c12] | 2377 |
| 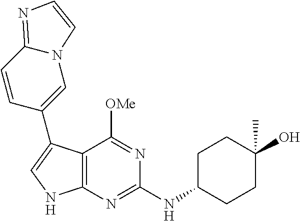 [SMILES: COc1nc(N[C@H]2CC[C@](C)(O)CC2)nc2[nH]cc(-c3ccc4nccn4c3)c12] | 2378 |
| 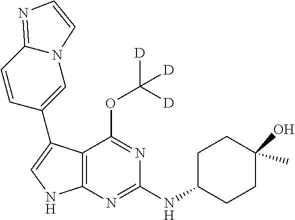 [SMILES: [2H]C([2H])([2H])Oc1nc(N[C@H]2CC[C@](C)(O)CC2)nc2[nH]cc(-c3ccc4nccn4c3)c12] | 2379 |
| 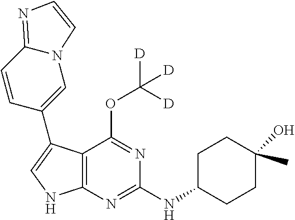 [SMILES: [2H]C([2H])([2H])Oc1nc(N[C@H]2CC[C@@](C)(O)CC2)nc2[nH]cc(-c3ccc4nccn4c3)c12] | 2380 |
| 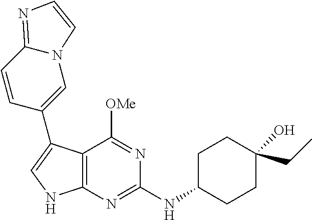 [SMILES: CC[C@]1(O)CC[C@@H](Nc2nc(OC)c3c(-c4ccc5nccn5c4)c[nH]c3n2)CC1] | 2381 |
| 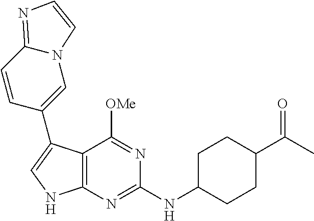 [SMILES: COc1nc(NC2CCC(C(C)=O)CC2)nc2[nH]cc(-c3ccc4nccn4c3)c12] | 2382 |
| 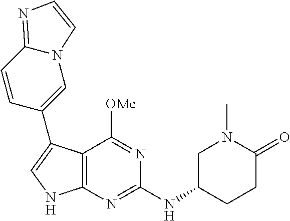 [SMILES: COc1nc(N[C@H]2CCC(=O)N(C)C2)nc2[nH]cc(-c3ccc4nccn4c3)c12] | 2383 |
| 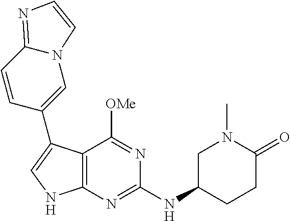 [SMILES: COc1nc(N[C@@H]2CCC(=O)N(C)C2)nc2[nH]cc(-c3ccc4nccn4c3)c12] | 2384 |
| 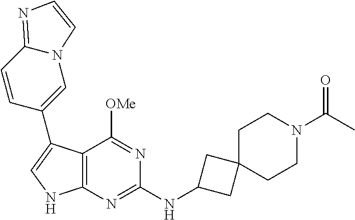 [SMILES: COc1nc(NC2CC3(CCN(C(C)=O)CC3)C2)nc2[nH]cc(-c3ccc4nccn4c3)c12] | 2385 |
| 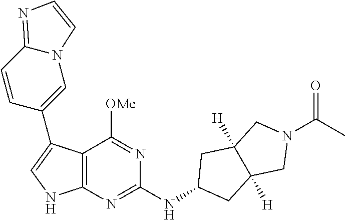 [SMILES: COc1nc(N[C@H]2C[C@@H]3CN(C(C)=O)C[C@@H]3C2)nc2[nH]cc(-c3ccc4nccn4c3)c12] | 2386 |
| 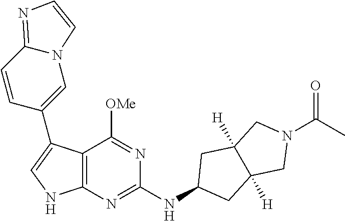 [SMILES: COc1nc(N[C@@H]2C[C@@H]3CN(C(C)=O)C[C@@H]3C2)nc2[nH]cc(-c3ccc4nccn4c3)c12] | 2387 |
| 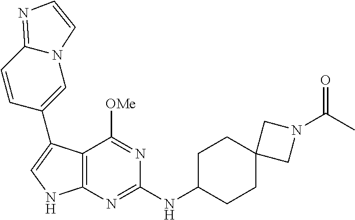 [SMILES: COc1nc(NC2CCC3(CC2)CN(C(C)=O)C3)nc2[nH]cc(-c3ccc4nccn4c3)c12] | 2388 |
| 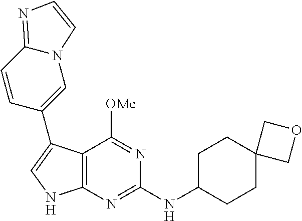 [SMILES: COc1nc(NC2CCC3(CC2)COC3)nc2[nH]cc(-c3ccc4nccn4c3)c12] | 2389 |
| 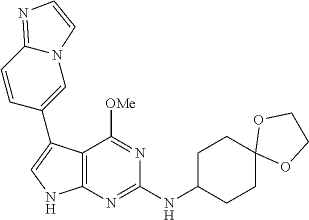 [SMILES: COc1nc(NC2CCC3(CC2)OCCO3)nc2[nH]cc(-c3ccc4nccn4c3)c12] | 2390 |
| 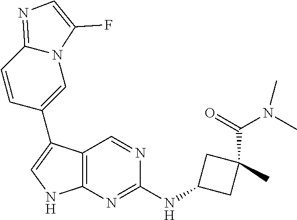 [SMILES: CN(C)C(=O)[C@]1(C)C[C@H](Nc2ncc3c(-c4ccc5ncc(F)n5c4)c[nH]c3n2)C1] | 2391 |
| 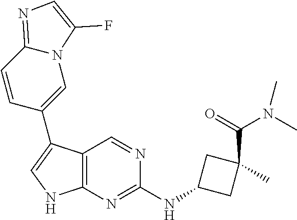 [SMILES: CN(C)C(=O)[C@]1(C)C[C@@H](Nc2ncc3c(-c4ccc5ncc(F)n5c4)c[nH]c3n2)C1] | 2392 |
| 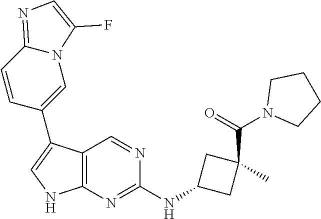 [SMILES: C[C@]1(C(=O)N2CCCC2)C[C@H](Nc2ncc3c(-c4ccc5ncc(F)n5c4)c[nH]c3n2)C1] | 2393 |
| 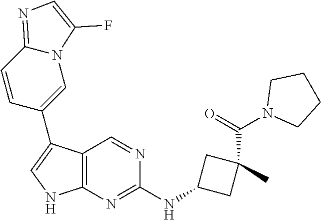 [SMILES: C[C@]1(C(=O)N2CCCC2)C[C@@H](Nc2ncc3c(-c4ccc5ncc(F)n5c4)c[nH]c3n2)C1] | 2394 |
| 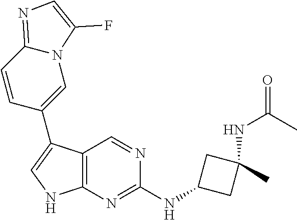 [SMILES: CC(=O)N[C@]1(C)C[C@H](Nc2ncc3c(-c4ccc5ncc(F)n5c4)c[nH]c3n2)C1] | 2395 |
| 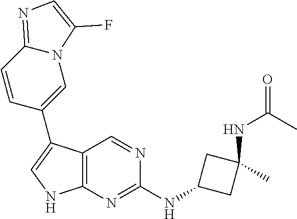 [SMILES: CC(=O)N[C@]1(C)C[C@@H](Nc2ncc3c(-c4ccc5ncc(F)n5c4)c[nH]c3n2)C1] | 2396 |
| 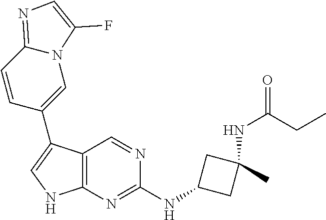 [SMILES: CCC(=O)N[C@]1(C)C[C@H](Nc2ncc3c(-c4ccc5ncc(F)n5c4)c[nH]c3n2)C1] | 2397 |
| 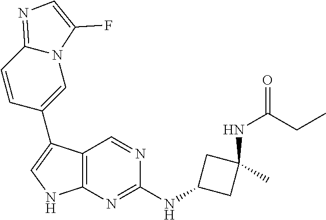 [SMILES: CCC(=O)N[C@]1(C)C[C@@H](Nc2ncc3c(-c4ccc5ncc(F)n5c4)c[nH]c3n2)C1] | 2398 |
| 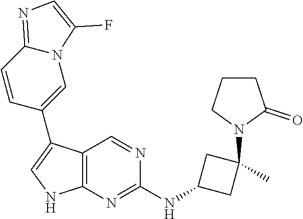 [SMILES: C[C@]1(N2CCCC2=O)C[C@H](Nc2ncc3c(-c4ccc5ncc(F)n5c4)c[nH]c3n2)C1] | 2399 |
| 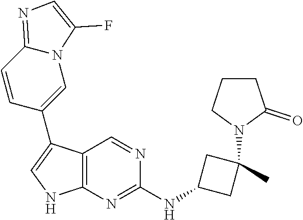 [SMILES: C[C@]1(N2CCCC2=O)C[C@@H](Nc2ncc3c(-c4ccc5ncc(F)n5c4)c[nH]c3n2)C1] | 2400 |
| 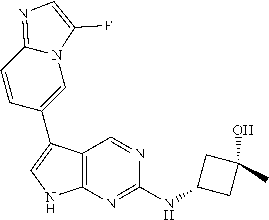 [SMILES: C[C@]1(O)C[C@@H](Nc2ncc3c(-c4ccc5ncc(F)n5c4)c[nH]c3n2)C1] | 2401 |
| 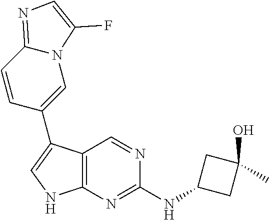 [SMILES: C[C@]1(O)C[C@H](Nc2ncc3c(-c4ccc5ncc(F)n5c4)c[nH]c3n2)C1] | 2402 |
| 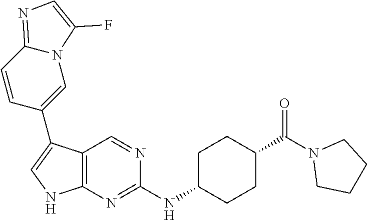 [SMILES: O=C([C@H]1CC[C@@H](Nc2ncc3c(-c4ccc5ncc(F)n5c4)c[nH]c3n2)CC1)N1CCCC1] | 2403 |
| 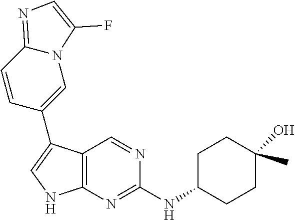 [SMILES: C[C@]1(O)CC[C@@H](Nc2ncc3c(-c4ccc5ncc(F)n5c4)c[nH]c3n2)CC1] | 2404 |
| 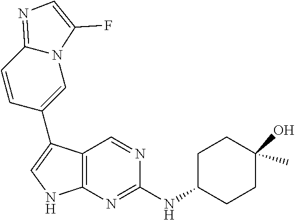 [SMILES: C[C@]1(O)CC[C@H](Nc2ncc3c(-c4ccc5ncc(F)n5c4)c[nH]c3n2)CC1] | 2405 |
| 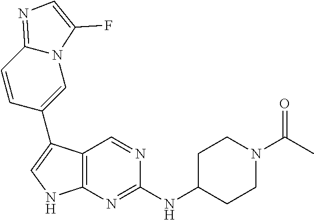 [SMILES: CC(=O)N1CCC(Nc2ncc3c(-c4ccc5ncc(F)n5c4)c[nH]c3n2)CC1] | 2406 |
| 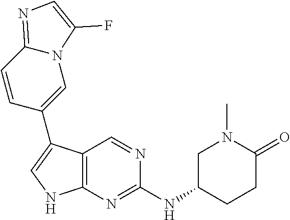 [SMILES: CN1C[C@@H](Nc2ncc3c(-c4ccc5ncc(F)n5c4)c[nH]c3n2)CCC1=O] | 2407 |
| 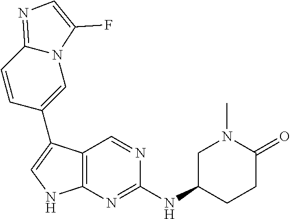 [SMILES: CN1C[C@H](Nc2ncc3c(-c4ccc5ncc(F)n5c4)c[nH]c3n2)CCC1=O] | 2408 |
| 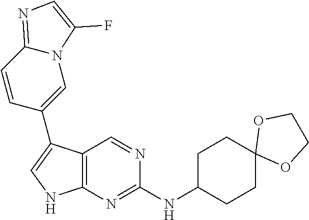 [SMILES: Fc1cnc2ccc(-c3c[nH]c4nc(NC5CCC6(CC5)OCCO6)ncc34)cn12] | 2409 |
| 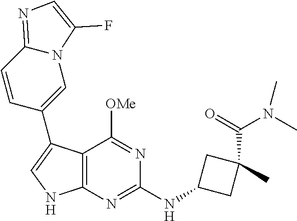 [SMILES: COc1nc(N[C@H]2C[C@@](C)(C(=O)N(C)C)C2)nc2[nH]cc(-c3ccc4ncc(F)n4c3)c12] | 2410 |
| 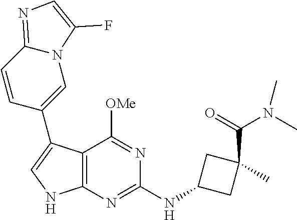 [SMILES: COc1nc(N[C@H]2C[C@](C)(C(=O)N(C)C)C2)nc2[nH]cc(-c3ccc4ncc(F)n4c3)c12] | 2411 |
| 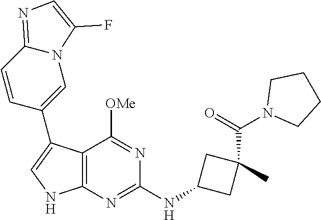 [SMILES: COc1nc(N[C@H]2C[C@@](C)(C(=O)N3CCCC3)C2)nc2[nH]cc(-c3ccc4ncc(F)n4c3)c12] | 2412 |
| 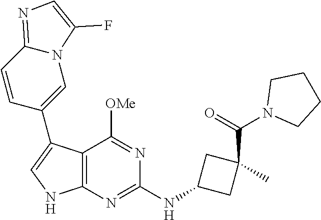 [SMILES: COc1nc(N[C@H]2C[C@](C)(C(=O)N3CCCC3)C2)nc2[nH]cc(-c3ccc4ncc(F)n4c3)c12] | 4213 |
| 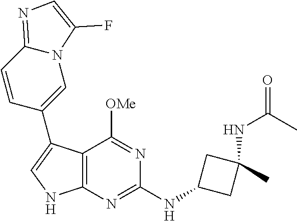 [SMILES: COc1nc(N[C@H]2C[C@@](C)(NC(C)=O)C2)nc2[nH]cc(-c3ccc4ncc(F)n4c3)c12] | 2414 |
| 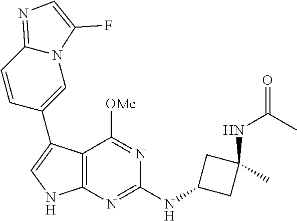 [SMILES: COc1nc(N[C@H]2C[C@](C)(NC(C)=O)C2)nc2[nH]cc(-c3ccc4ncc(F)n4c3)c12] | 2415 |
| 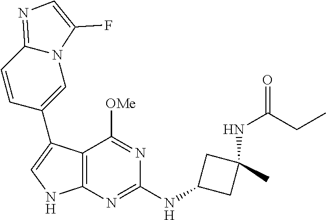 [SMILES: CCC(=O)N[C@]1(C)C[C@H](Nc2nc(OC)c3c(-c4ccc5ncc(F)n5c4)c[nH]c3n2)C1] | 2416 |
| 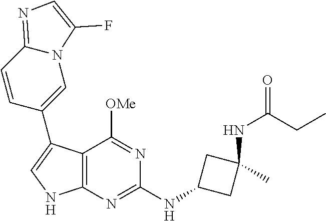 [SMILES: CCC(=O)N[C@]1(C)C[C@@H](Nc2nc(OC)c3c(-c4ccc5ncc(F)n5c4)c[nH]c3n2)C1] | 2417 |
| 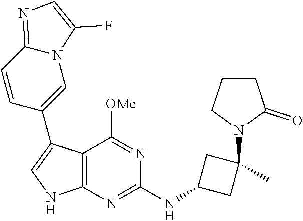 [SMILES: COc1nc(N[C@H]2C[C@](C)(N3CCCC3=O)C2)nc2[nH]cc(-c3ccc4ncc(F)n4c3)c12] | 2418 |
| 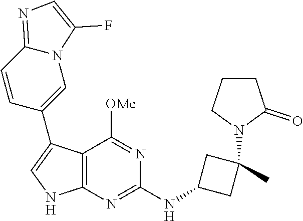 [SMILES: COc1nc(N[C@H]2C[C@@](C)(N3CCCC3=O)C2)nc2[nH]cc(-c3ccc4ncc(F)n4c3)c12] | 2419 |
| 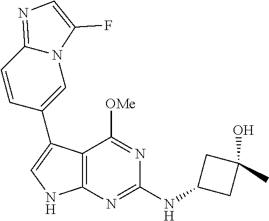 [SMILES: COc1nc(N[C@H]2C[C@@](C)(O)C2)nc2[nH]cc(-c3ccc4ncc(F)n4c3)c12] | 2420 |
| 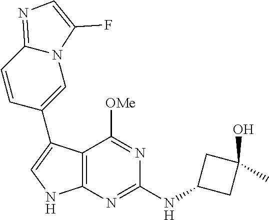 [SMILES: COc1nc(N[C@H]2C[C@](C)(O)C2)nc2[nH]cc(-c3ccc4ncc(F)n4c3)c12] | 2421 |
| 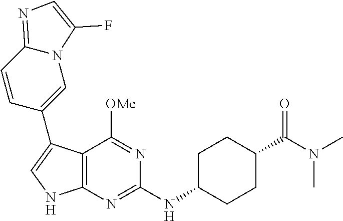 [SMILES: COc1nc(N[C@H]2CC[C@@H](C(=O)N(C)C)CC2)nc2[nH]cc(-c3ccc4ncc(F)n4c3)c12] | 2422 |
| 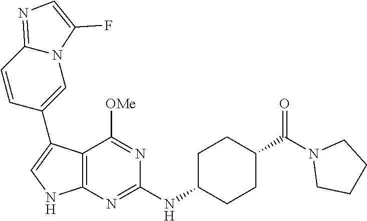 [SMILES: COc1nc(N[C@H]2CC[C@@H](C(=O)N3CCCC3)CC2)nc2[nH]cc(-c3ccc4ncc(F)n4c3)c12] | 2423 |
| 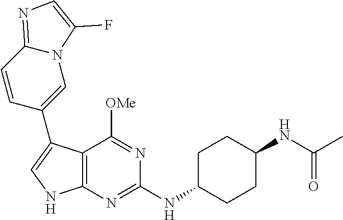 [SMILES: COc1nc(N[C@H]2CC[C@H](NC(C)=O)CC2)nc2[nH]cc(-c3ccc4ncc(F)n4c3)c12] | 2424 |
| 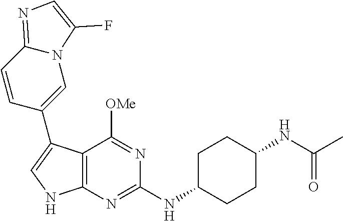 [SMILES: COc1nc(N[C@H]2CC[C@@H](NC(C)=O)CC2)nc2[nH]cc(-c3ccc4ncc(F)n4c3)c12] | 2425 |
| 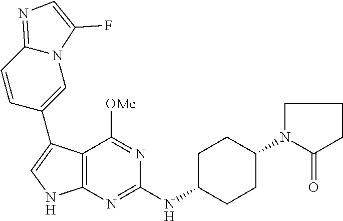 [SMILES: COc1nc(N[C@H]2CC[C@@H](N3CCCC3=O)CC2)nc2[nH]cc(-c3ccc4ncc(F)n4c3)c12] | 2426 |
| 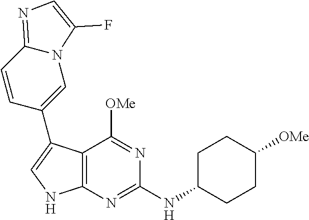 [SMILES: COc1nc(N[C@H]2CC[C@@H](OC)CC2)nc2[nH]cc(-c3ccc4ncc(F)n4c3)c12] | 2427 |
| 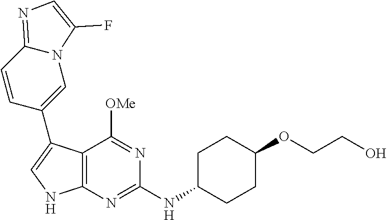 [SMILES: COc1nc(N[C@H]2CC[C@H](OCCO)CC2)nc2[nH]cc(-c3ccc4ncc(F)n4c3)c12] | 2428 |
| 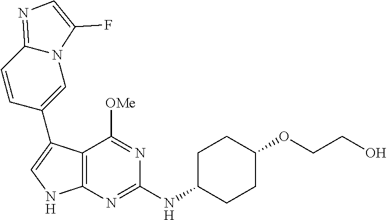 [SMILES: COc1nc(N[C@H]2CC[C@@H](OCCO)CC2)nc2[nH]cc(-c3ccc4ncc(F)n4c3)c12] | 2429 |
| 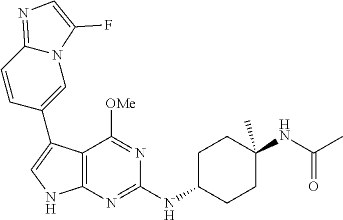 [SMILES: COc1nc(N[C@H]2CC[C@](C)(NC(C)=O)CC2)nc2[nH]cc(-c3ccc4ncc(F)n4c3)c12] | 2430 |
| 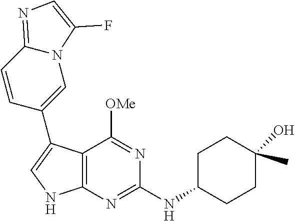 [SMILES: COc1nc(N[C@H]2CC[C@@](C)(O)CC2)nc2[nH]cc(-c3ccc4ncc(F)n4c3)c12] | 2431 |
| 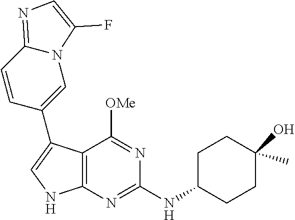 [SMILES: COc1nc(N[C@H]2CC[C@](C)(O)CC2)nc2[nH]cc(-c3ccc4ncc(F)n4c3)c12] | 2432 |
| 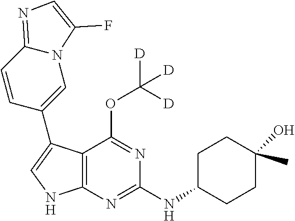 [SMILES: [2H]C([2H])([2H])Oc1nc(N[C@H]2CC[C@@](C)(O)CC2)nc2[nH]cc(-c3ccc4ncc(F)n4c3)c12] | 2433 |
| 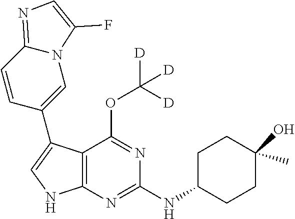 [SMILES: [2H]C([2H])([2H])Oc1nc(N[C@H]2CC[C@](C)(O)CC2)nc2[nH]cc(-c3ccc4ncc(F)n4c3)c12] | 2434 |
| 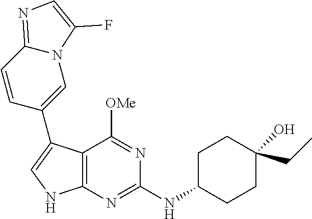 [SMILES: CC[C@]1(O)CC[C@@H](Nc2nc(OC)c3c(-c4ccc5ncc(F)n5c4)c[nH]c3n2)CC1] | 2435 |
| 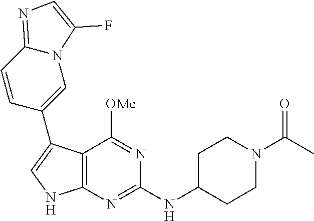 [SMILES: COc1nc(NC2CCN(C(C)=O)CC2)nc2[nH]cc(-c3ccc4ncc(F)n4c3)c12] | 2436 |
| 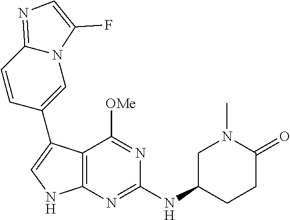 [SMILES: COc1nc(N[C@@H]2CCC(=O)N(C)C2)nc2[nH]cc(-c3ccc4ncc(F)n4c3)c12] | 2437 |
| 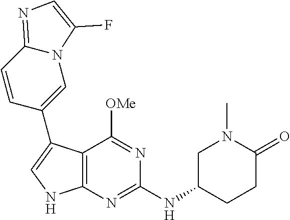 [SMILES: COc1nc(N[C@H]2CCC(=O)N(C)C2)nc2[nH]cc(-c3ccc4ncc(F)n4c3)c12] | 2438 |
| 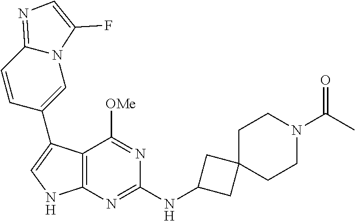 [SMILES: COc1nc(NC2CC3(CCN(C(C)=O)CC3)C2)nc2[nH]cc(-c3ccc4ncc(F)n4c3)c12] | 2439 |
| 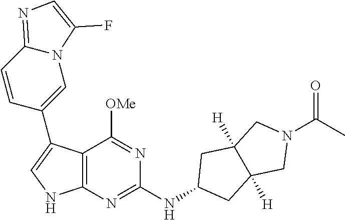 [SMILES: COc1nc(N[C@H]2C[C@@H]3CN(C(C)=O)C[C@@H]3C2)nc2[nH]cc(-c3ccc4ncc(F)n4c3)c12] | 2440 |
| 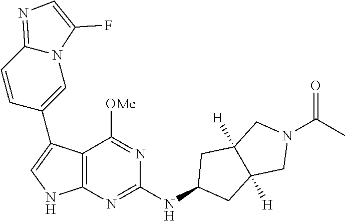 [SMILES: COc1nc(N[C@@H]2C[C@@H]3CN(C(C)=O)C[C@@H]3C2)nc2[nH]cc(-c3ccc4ncc(F)n4c3)c12] | 2441 |
| 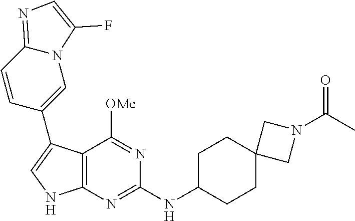 [SMILES: COc1nc(NC2CCC3(CC2)CN(C(C)=O)C3)nc2[nH]cc(-c3ccc4ncc(F)n4c3)c12] | 2442 |
| 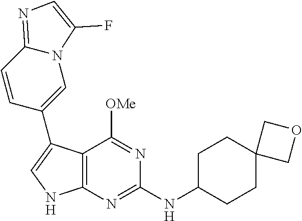 [SMILES: COc1nc(NC2CCC3(CC2)COC3)nc2[nH]cc(-c3ccc4ncc(F)n4c3)c12] | 2443 |
| 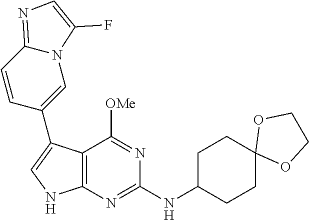 [SMILES: COc1nc(NC2CCC3(CC2)OCCO3)nc2[nH]cc(-c3ccc4ncc(F)n4c3)c12] | 2444 |
| 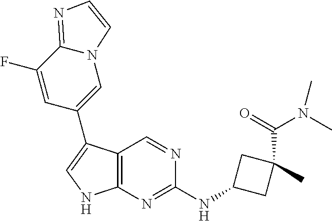 [SMILES: CN(C)C(=O)[C@]1(C)C[C@H](Nc2ncc3c(-c4cc(F)c5nccn5c4)c[nH]c3n2)C1] | 2445 |
| 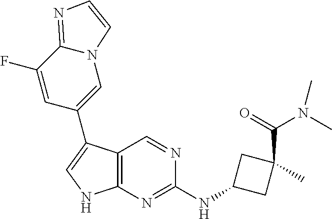 [SMILES: CN(C)C(=O)[C@]1(C)C[C@@H](Nc2ncc3c(-c4cc(F)c5nccn5c4)c[nH]c3n2)C1] | 2446 |
| 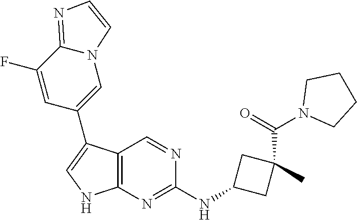 [SMILES: C[C@]1(C(=O)N2CCCC2)C[C@@H](Nc2ncc3c(-c4cc(F)c5nccn5c4)c[nH]c3n2)C1] | 2447 |
| 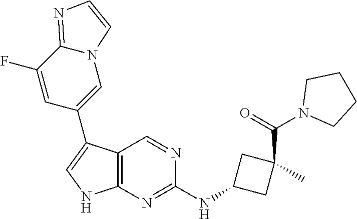 [SMILES: C[C@]1(C(=O)N2CCCC2)C[C@H](Nc2ncc3c(-c4cc(F)c5nccn5c4)c[nH]c3n2)C1] | 2448 |
| 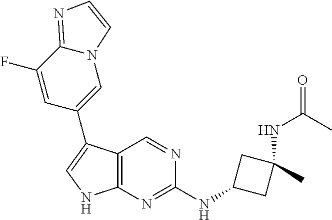 [SMILES: CC(=O)N[C@]1(C)C[C@H](Nc2ncc3c(-c4cc(F)c5nccn5c4)c[nH]c3n2)C1] | 2449 |
| 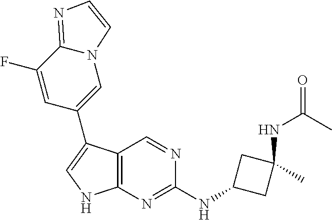 [SMILES: CC(=O)N[C@]1(C)C[C@@H](Nc2ncc3c(-c4cc(F)c5nccn5c4)c[nH]c3n2)C1] | 2450 |
| 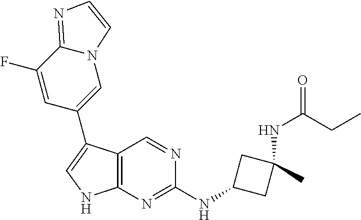 [SMILES: CCC(=O)N[C@]1(C)C[C@H](Nc2ncc3c(-c4cc(F)c5nccn5c4)c[nH]c3n2)C1] | 2451 |
| 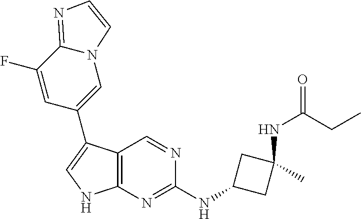 [SMILES: CCC(=O)N[C@]1(C)C[C@@H](Nc2ncc3c(-c4cc(F)c5nccn5c4)c[nH]c3n2)C1] | 2452 |
| 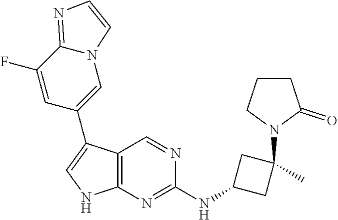 [SMILES: C[C@]1(N2CCCC2=O)C[C@H](Nc2ncc3c(-c4cc(F)c5nccn5c4)c[nH]c3n2)C1] | 2453 |
| 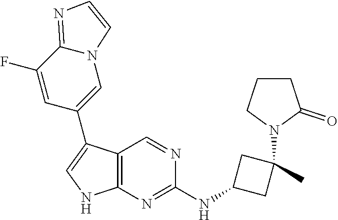 [SMILES: C[C@]1(N2CCCC2=O)C[C@@H](Nc2ncc3c(-c4cc(F)c5nccn5c4)c[nH]c3n2)C1] | 2454 |
| 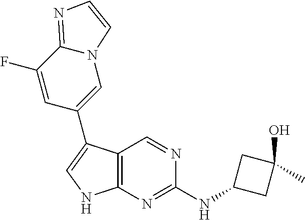 [SMILES: C[C@]1(O)C[C@H](Nc2ncc3c(-c4cc(F)c5nccn5c4)c[nH]c3n2)C1] | 2455 |
| 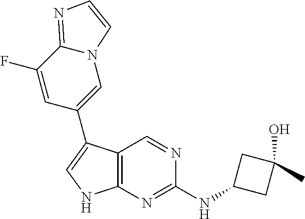 [SMILES: C[C@]1(O)C[C@@H](Nc2ncc3c(-c4cc(F)c5nccn5c4)c[nH]c3n2)C1] | 2456 |
| 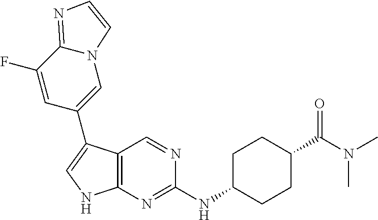 [SMILES: CN(C)C(=O)[C@H]1CC[C@@H](Nc2ncc3c(-c4cc(F)c5nccn5c4)c[nH]c3n2)CC1] | 2457 |
| 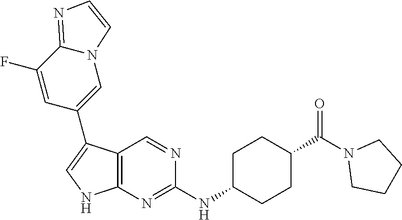 [SMILES: O=C([C@H]1CC[C@@H](Nc2ncc3c(-c4cc(F)c5nccn5c4)c[nH]c3n2)CC1)N1CCCC1] | 2458 |
| 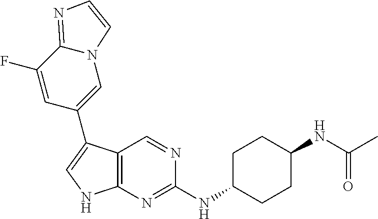 [SMILES: CC(=O)N[C@H]1CC[C@H](Nc2ncc3c(-c4cc(F)c5nccn5c4)c[nH]c3n2)CC1] | 2459 |
| 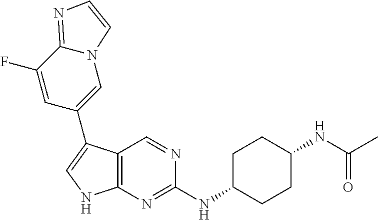 [SMILES: CC(=O)N[C@H]1CC[C@@H](Nc2ncc3c(-c4cc(F)c5nccn5c4)c[nH]c3n2)CC1] | 2460 |
| 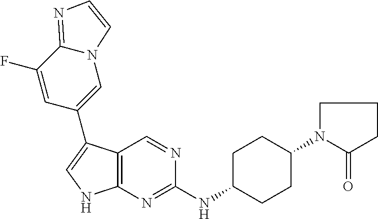 [SMILES: O=C1CCCN1[C@H]1CC[C@@H](Nc2ncc3c(-c4cc(F)c5nccn5c4)c[nH]c3n2)CC1] | 2461 |
| 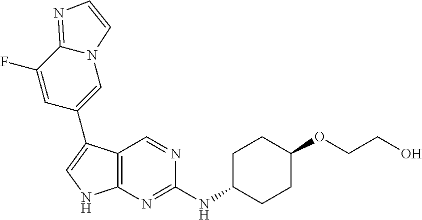 [SMILES: OCCO[C@H]1CC[C@H](Nc2ncc3c(-c4cc(F)c5nccn5c4)c[nH]c3n2)CC1] | 2462 |
| 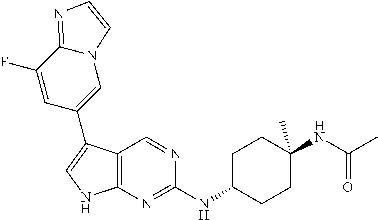 [SMILES: CC(=O)N[C@]1(C)CC[C@@H](Nc2ncc3c(-c4cc(F)c5nccn5c4)c[nH]c3n2)CC1] | 2463 |
| 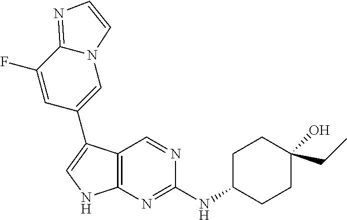 [SMILES: CC[C@]1(O)CC[C@@H](Nc2ncc3c(-c4cc(F)c5nccn5c4)c[nH]c3n2)CC1] | 2464 |
| 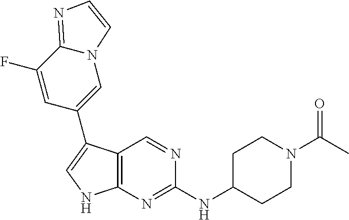 [SMILES: CC(=O)N1CCC(Nc2ncc3c(-c4cc(F)c5nccn5c4)c[nH]c3n2)CC1] | 2465 |
| 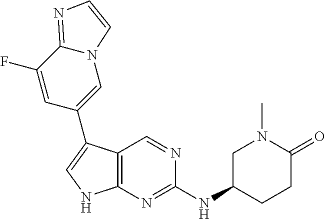 [SMILES: CN1C[C@H](Nc2ncc3c(-c4cc(F)c5nccn5c4)c[nH]c3n2)CCC1=O] | 2466 |
| 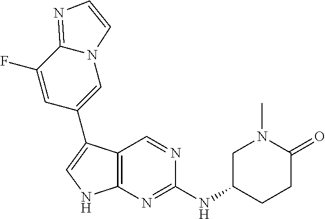 [SMILES: CN1C[C@@H](Nc2ncc3c(-c4cc(F)c5nccn5c4)c[nH]c3n2)CCC1=O] | 2467 |
| 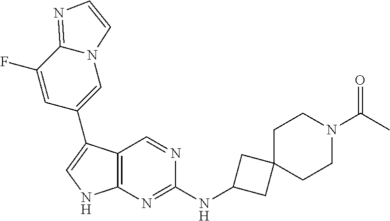 [SMILES: CC(=O)N1CCC2(CC1)CC(Nc1ncc3c(-c4cc(F)c5nccn5c4)c[nH]c3n1)C2] | 2468 |
| 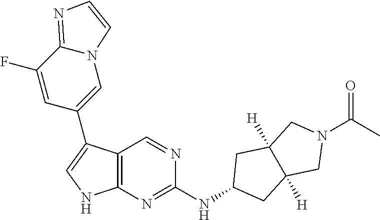 [SMILES: CC(=O)N1C[C@H]2C[C@H](Nc3ncc4c(-c5cc(F)c6nccn6c5)c[nH]c4n3)C[C@H]2C1] | 2469 |
| 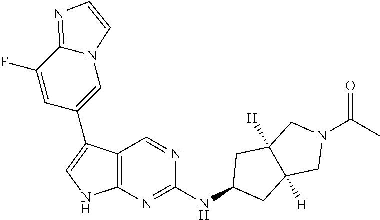 [SMILES: CC(=O)N1C[C@H]2C[C@@H](Nc3ncc4c(-c5cc(F)c6nccn6c5)c[nH]c4n3)C[C@H]2C1] | 2470 |
| 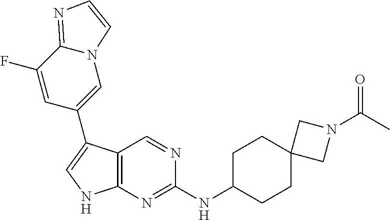 [SMILES: CC(=O)N1CC2(CCC(Nc3ncc4c(-c5cc(F)c6nccn6c5)c[nH]c4n3)CC2)C1] | 2471 |
| 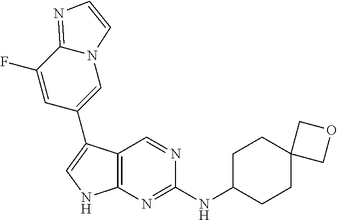 [SMILES: Fc1cc(-c2c[nH]c3nc(NC4CCC5(CC4)COC5)ncc23)cn2ccnc12] | 2472 |
| 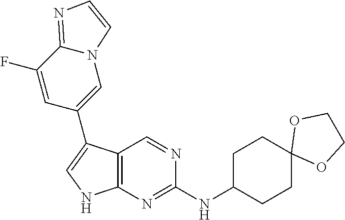 [SMILES: Fc1cc(-c2c[nH]c3nc(NC4CCC5(CC4)OCCO5)ncc23)cn2ccnc12] | 2473 |
| 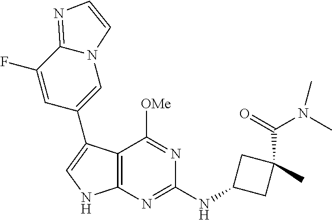 [SMILES: COc1nc(N[C@H]2C[C@@](C)(C(=O)N(C)C)C2)nc2[nH]cc(-c3cc(F)c4nccn4c3)c12] | 2474 |
| 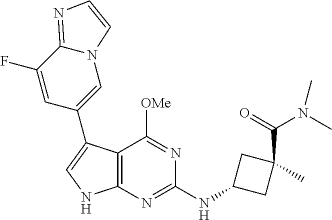 [SMILES: COc1nc(N[C@H]2C[C@](C)(C(=O)N(C)C)C2)nc2[nH]cc(-c3cc(F)c4nccn4c3)c12] | 2475 |
| 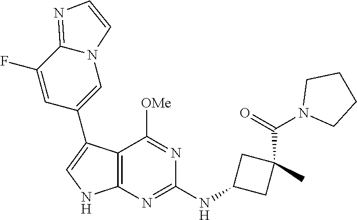 [SMILES: COc1nc(N[C@H]2C[C@@](C)(C(=O)N3CCCC3)C2)nc2[nH]cc(-c3cc(F)c4nccn4c3)c12] | 2476 |
| 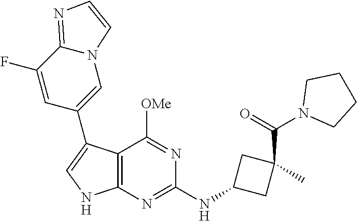 [SMILES: COc1nc(N[C@H]2C[C@](C)(C(=O)N3CCCC3)C2)nc2[nH]cc(-c3cc(F)c4nccn4c3)c12] | 2477 |
| 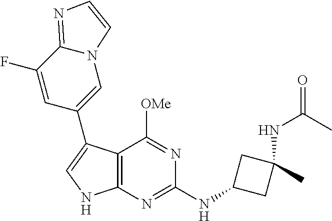 [SMILES: COc1nc(N[C@H]2C[C@@](C)(NC(C)=O)C2)nc2[nH]cc(-c3cc(F)c4nccn4c3)c12] | 2478 |
| 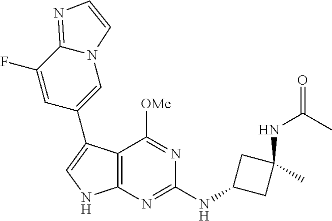 [SMILES: COc1nc(N[C@H]2C[C@](C)(NC(C)=O)C2)nc2[nH]cc(-c3cc(F)c4nccn4c3)c12] | 2479 |
| 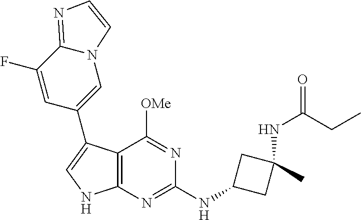 [SMILES: CCC(=O)N[C@]1(C)C[C@H](Nc2nc(OC)c3c(-c4cc(F)c5nccn5c4)c[nH]c3n2)C1] | 2480 |
| 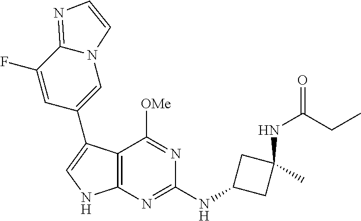 [SMILES: CCC(=O)N[C@]1(C)C[C@@H](Nc2nc(OC)c3c(-c4cc(F)c5nccn5c4)c[nH]c3n2)C1] | 2481 |
| 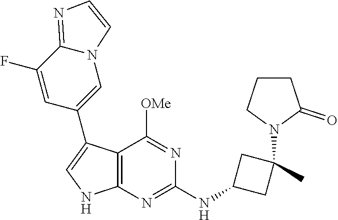 [SMILES: COc1nc(N[C@H]2C[C@@](C)(N3CCCC3=O)C2)nc2[nH]cc(-c3cc(F)c4nccn4c3)c12] | 2482 |
| 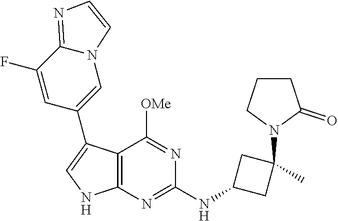 [SMILES: COc1nc(N[C@H]2C[C@](C)(N3CCCC3=O)C2)nc2[nH]cc(-c3cc(F)c4nccn4c3)c12] | 2483 |
| 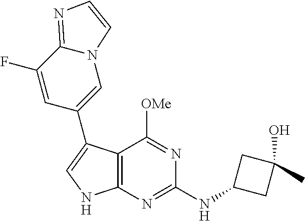 [SMILES: COc1nc(N[C@H]2C[C@@](C)(O)C2)nc2[nH]cc(-c3cc(F)c4nccn4c3)c12] | 2484 |
| 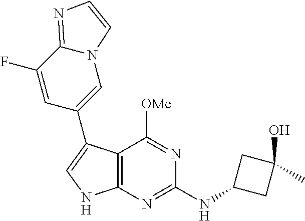 [SMILES: COc1nc(N[C@H]2C[C@](C)(O)C2)nc2[nH]cc(-c3cc(F)c4nccn4c3)c12] | 2485 |
| 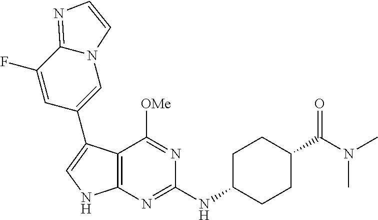 [SMILES: COc1nc(N[C@H]2CC[C@@H](C(=O)N(C)C)CC2)nc2[nH]cc(-c3cc(F)c4nccn4c3)c12] | 2486 |
| 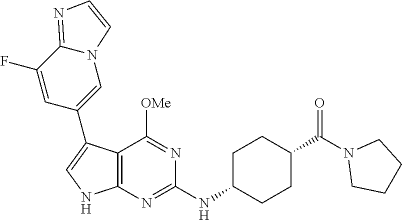 [SMILES: COc1nc(N[C@H]2CC[C@@H](C(=O)N3CCCC3)CC2)nc2[nH]cc(-c3cc(F)c4nccn4c3)c12] | 2487 |
| 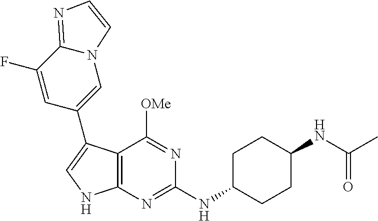 [SMILES: COc1nc(N[C@H]2CC[C@H](NC(C)=O)CC2)nc2[nH]cc(-c3cc(F)c4nccn4c3)c12] | 2488 |
| 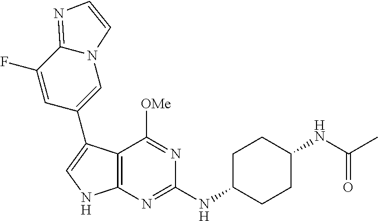 [SMILES: COc1nc(N[C@H]2CC[C@@H](NC(C)=O)CC2)nc2[nH]cc(-c3cc(F)c4nccn4c3)c12] | 2489 |
| 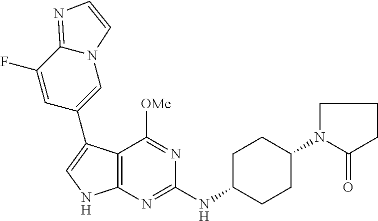 [SMILES: COc1nc(N[C@H]2CC[C@@H](N3CCCC3=O)CC2)nc2[nH]cc(-c3cc(F)c4nccn4c3)c12] | 2490 |
| 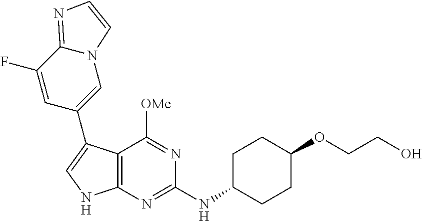 [SMILES: COc1nc(N[C@H]2CC[C@H](OCCO)CC2)nc2[nH]cc(-c3cc(F)c4nccn4c3)c12] | 2491 |
| 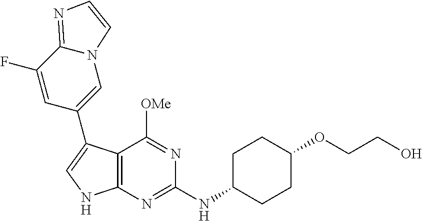 [SMILES: COc1nc(N[C@H]2CC[C@@H](OCCO)CC2)nc2[nH]cc(-c3cc(F)c4nccn4c3)c12] | 2492 |
| 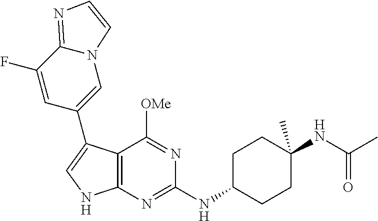 [SMILES: COc1nc(N[C@H]2CC[C@](C)(NC(C)=O)CC2)nc2[nH]cc(-c3cc(F)c4nccn4c3)c12] | 2493 |
| 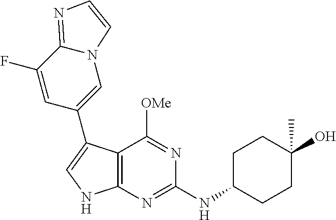 [SMILES: COc1nc(N[C@H]2CC[C@](C)(O)CC2)nc2[nH]cc(-c3cc(F)c4nccn4c3)c12] | 2494 |
| 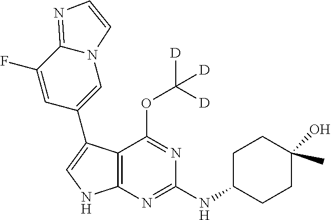 [SMILES: [2H]C([2H])([2H])Oc1nc(N[C@H]2CC[C@@](C)(O)CC2)nc2[nH]cc(-c3cc(F)c4nccn4c3)c12] | 2495 |
| 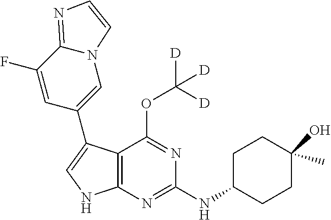 [SMILES: [2H]C([2H])([2H])Oc1nc(N[C@H]2CC[C@](C)(O)CC2)nc2[nH]cc(-c3cc(F)c4nccn4c3)c12] | 2496 |
| 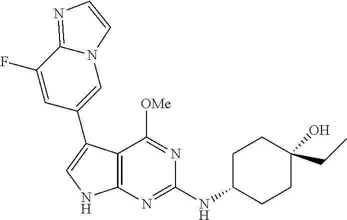 [SMILES: CC[C@]1(O)CC[C@@H](Nc2nc(OC)c3c(-c4cc(F)c5nccn5c4)c[nH]c3n2)CC1] | 2497 |
| 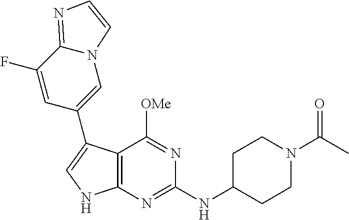 [SMILES: COc1nc(NC2CCN(C(C)=O)CC2)nc2[nH]cc(-c3cc(F)c4nccn4c3)c12] | 2498 |
| 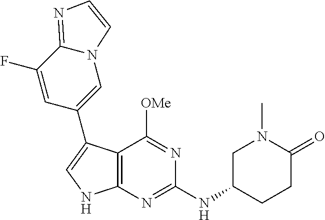 [SMILES: COc1nc(N[C@H]2CCC(=O)N(C)C2)nc2[nH]cc(-c3cc(F)c4nccn4c3)c12] | 2499 |
| 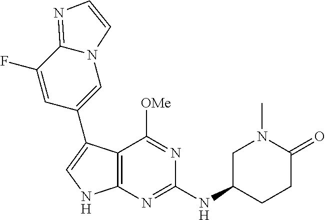 [SMILES: COc1nc(N[C@@H]2CCC(=O)N(C)C2)nc2[nH]cc(-c3cc(F)c4nccn4c3)c12] | 2500 |
| 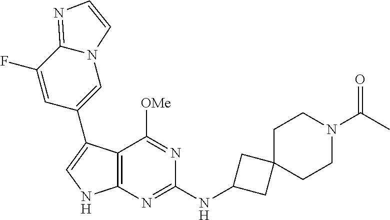 [SMILES: COc1nc(NC2CC3(CCN(C(C)=O)CC3)C2)nc2[nH]cc(-c3cc(F)c4nccn4c3)c12] | 2501 |
| 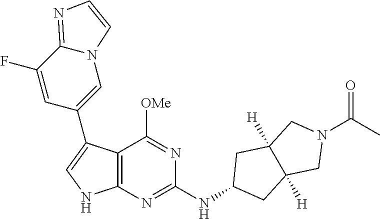 [SMILES: COc1nc(N[C@H]2C[C@@H]3CN(C(C)=O)C[C@@H]3C2)nc2[nH]cc(-c3cc(F)c4nccn4c3)c12] | 2502 |
| 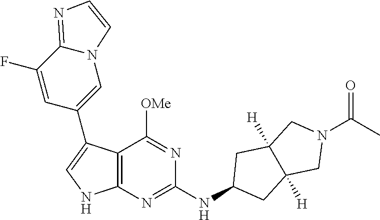 [SMILES: COc1nc(N[C@@H]2C[C@@H]3CN(C(C)=O)C[C@@H]3C2)nc2[nH]cc(-c3cc(F)c4nccn4c3)c12] | 2503 |
| 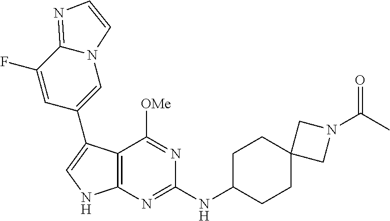 [SMILES: COc1nc(NC2CCC3(CC2)CN(C(C)=O)C3)nc2[nH]cc(-c3cc(F)c4nccn4c3)c12] | 2504 |
| 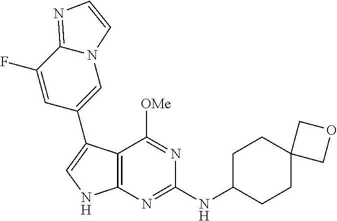 [SMILES: COc1nc(NC2CCC3(CC2)COC3)nc2[nH]cc(-c3cc(F)c4nccn4c3)c12] | 2505 |
| 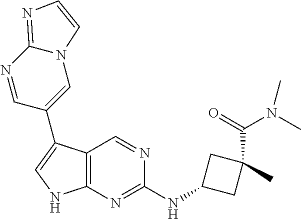 [SMILES: CN(C)C(=O)[C@]1(C)C[C@H](Nc2ncc3c(-c4cnc5nccn5c4)c[nH]c3n2)C1] | 2506 |
| 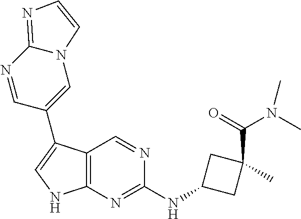 [SMILES: CN(C)C(=O)[C@]1(C)C[C@@H](Nc2ncc3c(-c4cnc5nccn5c4)c[nH]c3n2)C1] | 2507 |
| 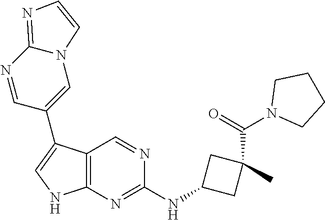 [SMILES: C[C@]1(C(=O)N2CCCC2)C[C@@H](Nc2ncc3c(-c4cnc5nccn5c4)c[nH]c3n2)C1] | 2508 |
| 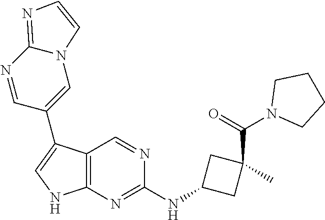 [SMILES: C[C@]1(C(=O)N2CCCC2)C[C@H](Nc2ncc3c(-c4cnc5nccn5c4)c[nH]c3n2)C1] | 2509 |
| 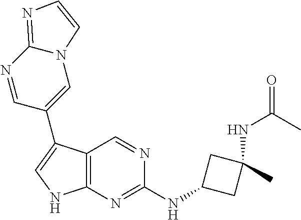 [SMILES: CC(=O)N[C@]1(C)C[C@H](Nc2ncc3c(-c4cnc5nccn5c4)c[nH]c3n2)C1] | 2510 |
| 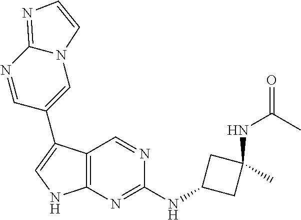 [SMILES: CC(=O)N[C@]1(C)C[C@@H](Nc2ncc3c(-c4cnc5nccn5c4)c[nH]c3n2)C1] | 2511 |
| 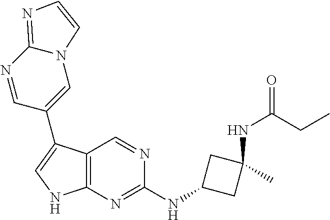 [SMILES: CCC(=O)N[C@]1(C)C[C@@H](Nc2ncc3c(-c4cnc5nccn5c4)c[nH]c3n2)C1] | 2512 |
| 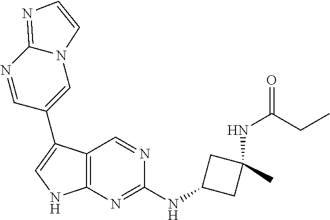 [SMILES: CCC(=O)N[C@]1(C)C[C@H](Nc2ncc3c(-c4cnc5nccn5c4)c[nH]c3n2)C1] | 2513 |
| 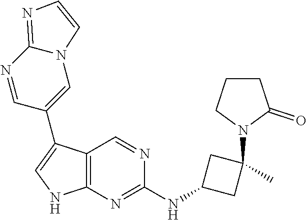 [SMILES: C[C@]1(N2CCCC2=O)C[C@H](Nc2ncc3c(-c4cnc5nccn5c4)c[nH]c3n2)C1] | 2514 |
| 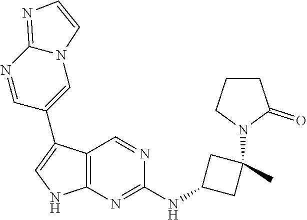 [SMILES: C[C@]1(N2CCCC2=O)C[C@@H](Nc2ncc3c(-c4cnc5nccn5c4)c[nH]c3n2)C1] | 2515 |
| 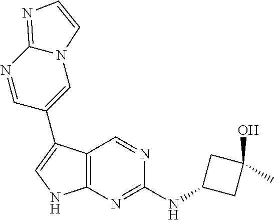 [SMILES: C[C@]1(O)C[C@H](Nc2ncc3c(-c4cnc5nccn5c4)c[nH]c3n2)C1] | 2516 |
| 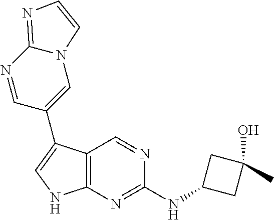 [SMILES: C[C@]1(O)C[C@@H](Nc2ncc3c(-c4cnc5nccn5c4)c[nH]c3n2)C1] | 2517 |
| 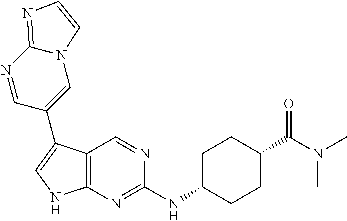 [SMILES: CN(C)C(=O)[C@H]1CC[C@@H](Nc2ncc3c(-c4cnc5nccn5c4)c[nH]c3n2)CC1] | 2518 |
| 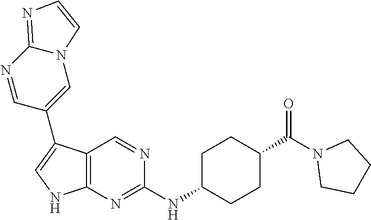 [SMILES: O=C([C@H]1CC[C@@H](Nc2ncc3c(-c4cnc5nccn5c4)c[nH]c3n2)CC1)N1CCCC1] | 2519 |
| 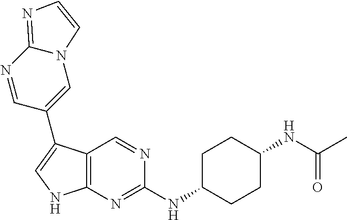 [SMILES: CC(=O)N[C@H]1CC[C@@H](Nc2ncc3c(-c4cnc5nccn5c4)c[nH]c3n2)CC1] | 2520 |
| 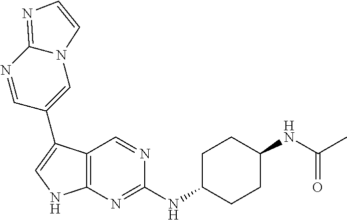 [SMILES: CC(=O)N[C@H]1CC[C@H](Nc2ncc3c(-c4cnc5nccn5c4)c[nH]c3n2)CC1] | 2521 |
| 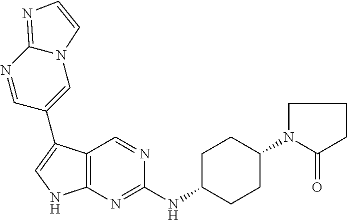 [SMILES: O=C1CCCN1[C@H]1CC[C@@H](Nc2ncc3c(-c4cnc5nccn5c4)c[nH]c3n2)CC1] | 2522 |
| 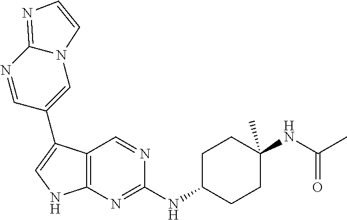 [SMILES: CC(=O)N[C@]1(C)CC[C@@H](Nc2ncc3c(-c4cnc5nccn5c4)c[nH]c3n2)CC1] | 2523 |
| 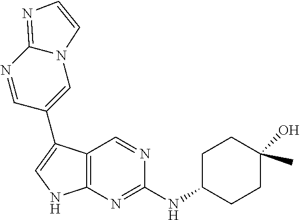 [SMILES: C[C@]1(O)CC[C@@H](Nc2ncc3c(-c4cnc5nccn5c4)c[nH]c3n2)CC1] | 2524 |
| 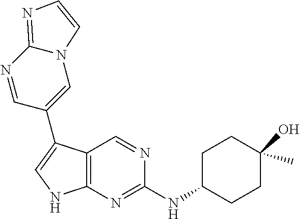 [SMILES: C[C@]1(O)CC[C@H](Nc2ncc3c(-c4cnc5nccn5c4)c[nH]c3n2)CC1] | 2525 |
| 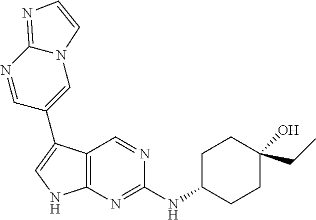 [SMILES: CC[C@]1(O)CC[C@@H](Nc2ncc3c(-c4cnc5nccn5c4)c[nH]c3n2)CC1] | 2526 |
| 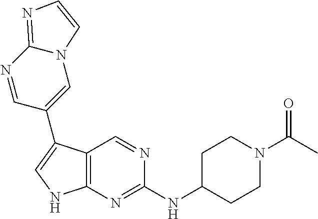 [SMILES: CC(=O)N1CCC(Nc2ncc3c(-c4cnc5nccn5c4)c[nH]c3n2)CC1] | 2527 |
| 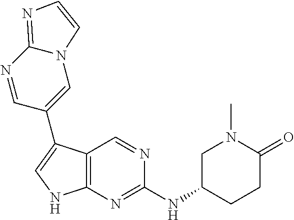 [SMILES: CN1C[C@@H](Nc2ncc3c(-c4cnc5nccn5c4)c[nH]c3n2)CCC1=O] | 2528 |
| 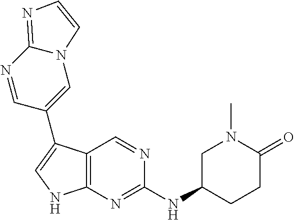 [SMILES: CN1C[C@H](Nc2ncc3c(-c4cnc5nccn5c4)c[nH]c3n2)CCC1=O] | 2529 |
| 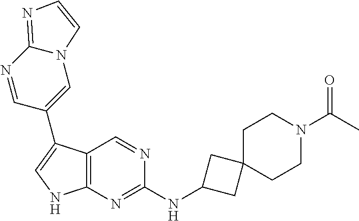 [SMILES: CC(=O)N1CCC2(CC1)CC(Nc1ncc3c(-c4cnc5nccn5c4)c[nH]c3n1)C2] | 2530 |
| 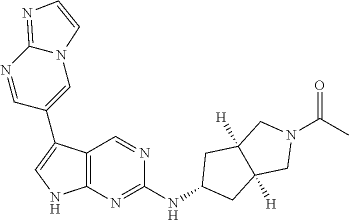 [SMILES: CC(=O)N1C[C@H]2C[C@H](Nc3ncc4c(-c5cnc6nccn6c5)c[nH]c4n3)C[C@H]2C1] | 2531 |
| 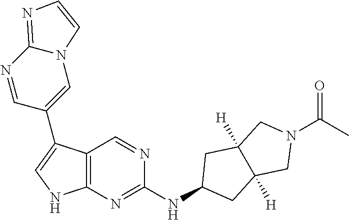 [SMILES: CC(=O)N1C[C@H]2C[C@@H](Nc3ncc4c(-c5cnc6nccn6c5)c[nH]c4n3)C[C@H]2C1] | 2532 |
| 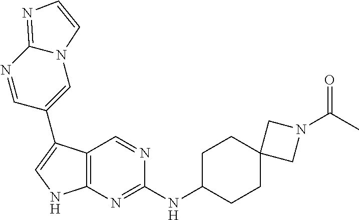 [SMILES: CC(=O)N1CC2(CCC(Nc3ncc4c(-c5cnc6nccn6c5)c[nH]c4n3)CC2)C1] | 2533 |
| 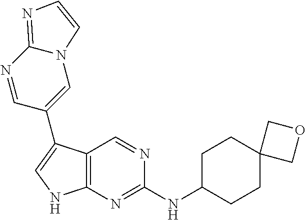 [SMILES: c1cn2cc(-c3c[nH]c4nc(NC5CCC6(CC5)COC6)ncc34)cnc2n1] | 2534 |
| 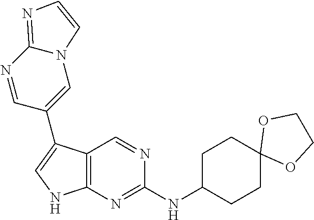 [SMILES: c1cn2cc(-c3c[nH]c4nc(NC5CCC6(CC5)OCCO6)ncc34)cnc2n1] | 2535 |
| 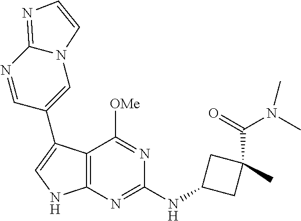 [SMILES: COc1nc(N[C@H]2C[C@@](C)(C(=O)N(C)C)C2)nc2[nH]cc(-c3cnc4nccn4c3)c12] | 2536 |
| 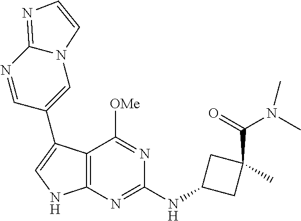 [SMILES: COc1nc(N[C@H]2C[C@](C)(C(=O)N(C)C)C2)nc2[nH]cc(-c3cnc4nccn4c3)c12] | 2537 |
| 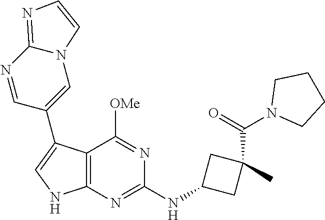 [SMILES: COc1nc(N[C@H]2C[C@@](C)(C(=O)N3CCCC3)C2)nc2[nH]cc(-c3cnc4nccn4c3)c12] | 2538 |
| 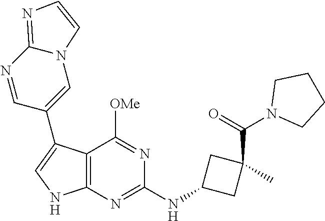 [SMILES: COc1nc(N[C@H]2C[C@](C)(C(=O)N3CCCC3)C2)nc2[nH]cc(-c3cnc4nccn4c3)c12] | 2539 |
| 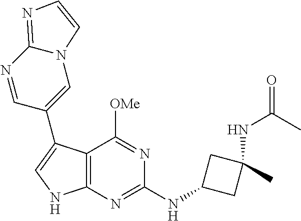 [SMILES: COc1nc(N[C@H]2C[C@@](C)(NC(C)=O)C2)nc2[nH]cc(-c3cnc4nccn4c3)c12] | 2540 |
| 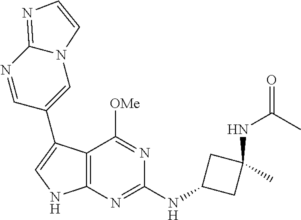 [SMILES: COc1nc(N[C@H]2C[C@](C)(NC(C)=O)C2)nc2[nH]cc(-c3cnc4nccn4c3)c12] | 2541 |
| 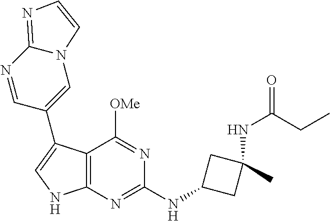 [SMILES: CCC(=O)N[C@]1(C)C[C@H](Nc2nc(OC)c3c(-c4cnc5nccn5c4)c[nH]c3n2)C1] | 2542 |
| 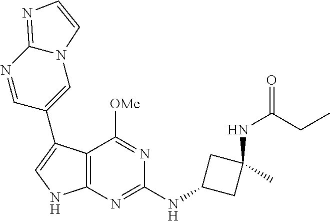 [SMILES: CCC(=O)N[C@]1(C)C[C@@H](Nc2nc(OC)c3c(-c4cnc5nccn5c4)c[nH]c3n2)C1] | 2543 |
| 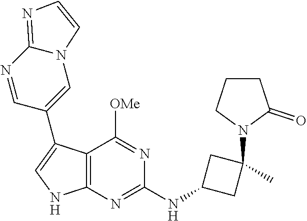 [SMILES: COc1nc(N[C@H]2C[C@](C)(N3CCCC3=O)C2)nc2[nH]cc(-c3cnc4nccn4c3)c12] | 2544 |
| 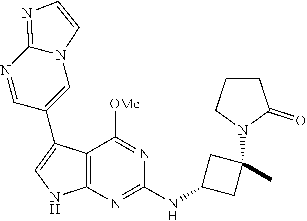 [SMILES: COc1nc(N[C@H]2C[C@@](C)(N3CCCC3=O)C2)nc2[nH]cc(-c3cnc4nccn4c3)c12] | 2545 |
| 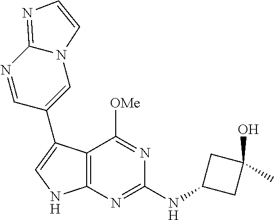 [SMILES: COc1nc(N[C@H]2C[C@](C)(O)C2)nc2[nH]cc(-c3cnc4nccn4c3)c12] | 2546 |
| 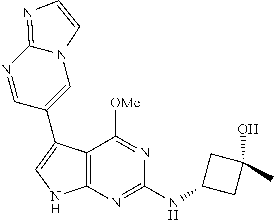 [SMILES: COc1nc(N[C@H]2C[C@@](C)(O)C2)nc2[nH]cc(-c3cnc4nccn4c3)c12] | 2547 |
| 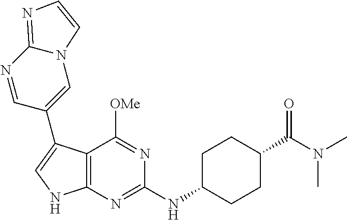 [SMILES: COc1nc(N[C@H]2CC[C@@H](C(=O)N(C)C)CC2)nc2[nH]cc(-c3cnc4nccn4c3)c12] | 2548 |
| 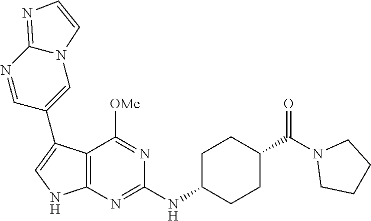 [SMILES: COc1nc(N[C@H]2CC[C@@H](C(=O)N3CCCC3)CC2)nc2[nH]cc(-c3cnc4nccn4c3)c12] | 2549 |
| 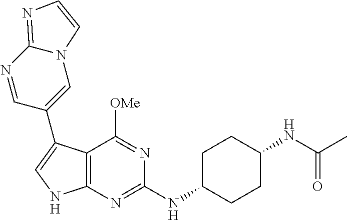 [SMILES: COc1nc(N[C@H]2CC[C@@H](NC(C)=O)CC2)nc2[nH]cc(-c3cnc4nccn4c3)c12] | 2550 |
| 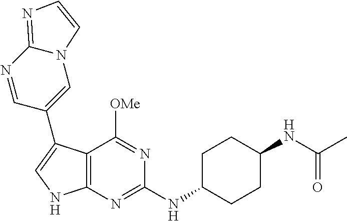 [SMILES: COc1nc(N[C@H]2CC[C@H](NC(C)=O)CC2)nc2[nH]cc(-c3cnc4nccn4c3)c12] | 2551 |
| 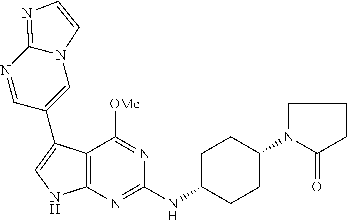 [SMILES: COc1nc(N[C@H]2CC[C@@H](N3CCCC3=O)CC2)nc2[nH]cc(-c3cnc4nccn4c3)c12] | 2552 |
| 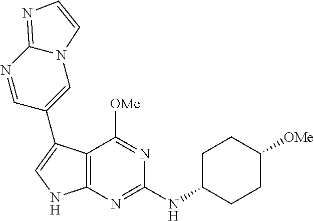 [SMILES: COc1nc(N[C@H]2CC[C@@H](OC)CC2)nc2[nH]cc(-c3cnc4nccn4c3)c12] | 2553 |
| 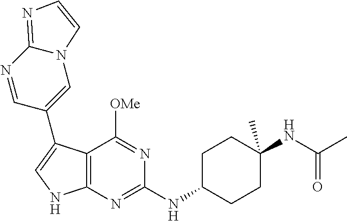 [SMILES: COc1nc(N[C@H]2CC[C@](C)(NC(C)=O)CC2)nc2[nH]cc(-c3cnc4nccn4c3)c12] | 2554 |
| 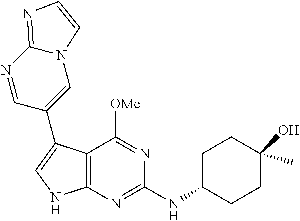 [SMILES: COc1nc(N[C@H]2CC[C@](C)(O)CC2)nc2[nH]cc(-c3cnc4nccn4c3)c12] | 2555 |
| 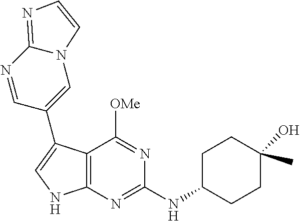 [SMILES: COc1nc(N[C@H]2CC[C@@](C)(O)CC2)nc2[nH]cc(-c3cnc4nccn4c3)c12] | 2556 |
| 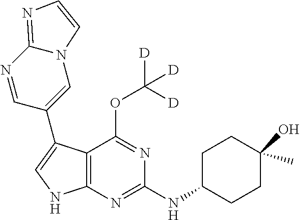 [SMILES: [2H]C([2H])([2H])Oc1nc(N[C@H]2CC[C@](C)(O)CC2)nc2[nH]cc(-c3cnc4nccn4c3)c12] | 2557 |
| 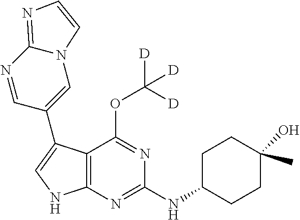 [SMILES: [2H]C([2H])([2H])Oc1nc(N[C@H]2CC[C@@](C)(O)CC2)nc2[nH]cc(-c3cnc4nccn4c3)c12] | 2558 |
| 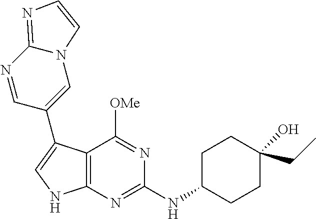 [SMILES: CC[C@]1(O)CC[C@@H](Nc2nc(OC)c3c(-c4cnc5nccn5c4)c[nH]c3n2)CC1] | 2559 |
| 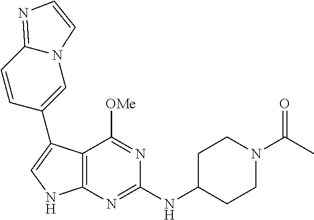 [SMILES: COc1nc(NC2CCN(C(C)=O)CC2)nc2[nH]cc(-c3ccc4nccn4c3)c12] | 2560 |
| 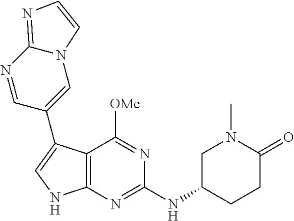 [SMILES: COc1nc(N[C@H]2CCC(=O)N(C)C2)nc2[nH]cc(-c3cnc4nccn4c3)c12] | 2561 |
| 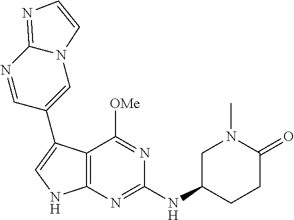 [SMILES: COc1nc(N[C@@H]2CCC(=O)N(C)C2)nc2[nH]cc(-c3cnc4nccn4c3)c12] | 2562 |
| 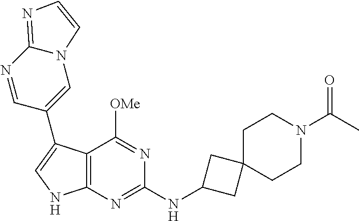 [SMILES: COc1nc(NC2CC3(CCN(C(C)=O)CC3)C2)nc2[nH]cc(-c3cnc4nccn4c3)c12] | 2563 |
| 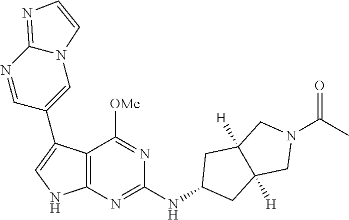 [SMILES: COc1nc(N[C@H]2C[C@@H]3CN(C(C)=O)C[C@@H]3C2)nc2[nH]cc(-c3cnc4nccn4c3)c12] | 2564 |
| 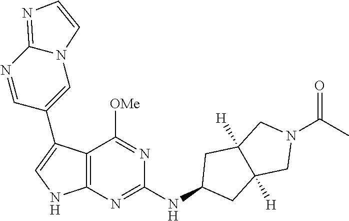 [SMILES: COc1nc(N[C@@H]2C[C@@H]3CN(C(C)=O)C[C@@H]3C2)nc2[nH]cc(-c3cnc4nccn4c3)c12] | 2565 |
| 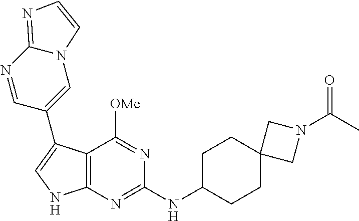 [SMILES: COc1nc(NC2CCC3(CC2)CN(C(C)=O)C3)nc2[nH]cc(-c3cnc4nccn4c3)c12] | 2566 |
| 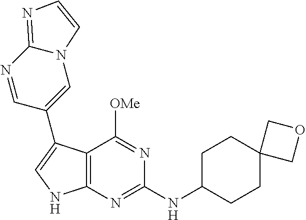 [SMILES: COc1nc(NC2CCC3(CC2)COC3)nc2[nH]cc(-c3cnc4nccn4c3)c12] | 2567 |
| 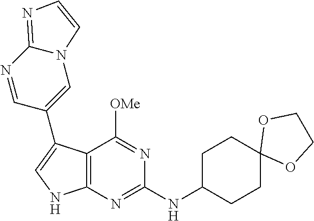 [SMILES: COc1nc(NC2CCC3(CC2)OCCO3)nc2[nH]cc(-c3cnc4nccn4c3)c12] | 2568 |
| 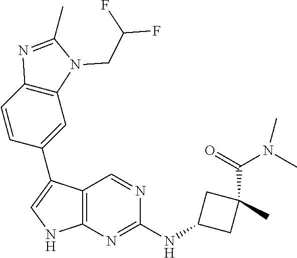 [SMILES: Cc1nc2ccc(-c3c[nH]c4nc(N[C@H]5C[C@@](C)(C(=O)N(C)C)C5)ncc34)cc2n1CC(F)F] | 2569 |
| 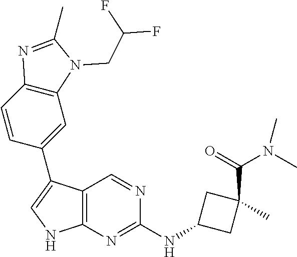 [SMILES: Cc1nc2ccc(-c3c[nH]c4nc(N[C@H]5C[C@](C)(C(=O)N(C)C)C5)ncc34)cc2n1CC(F)F] | 2570 |
| 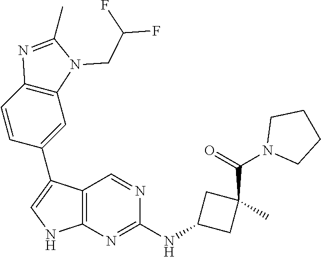 [SMILES: Cc1nc2ccc(-c3c[nH]c4nc(N[C@H]5C[C@](C)(C(=O)N6CCCC6)C5)ncc34)cc2n1CC(F)F] | 2571 |
| 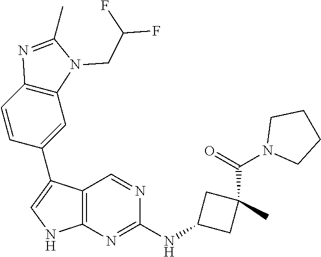 [SMILES: Cc1nc2ccc(-c3c[nH]c4nc(N[C@H]5C[C@@](C)(C(=O)N6CCCC6)C5)ncc34)cc2n1CC(F)F] | 2572 |
| 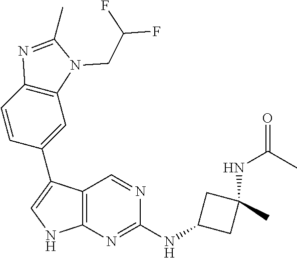 [SMILES: CC(=O)N[C@]1(C)C[C@H](Nc2ncc3c(-c4ccc5nc(C)n(CC(F)F)c5c4)c[nH]c3n2)C1] | 2573 |
| 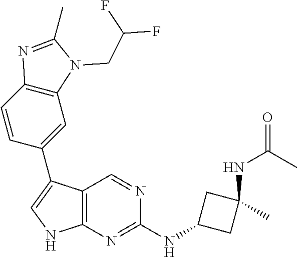 [SMILES: CC(=O)N[C@]1(C)C[C@@H](Nc2ncc3c(-c4ccc5nc(C)n(CC(F)F)c5c4)c[nH]c3n2)C1] | 2574 |
| 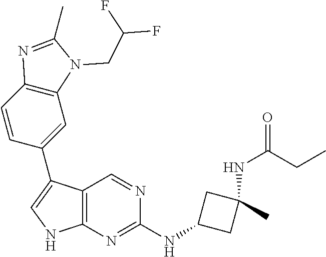 [SMILES: CCC(=O)N[C@]1(C)C[C@H](Nc2ncc3c(-c4ccc5nc(C)n(CC(F)F)c5c4)c[nH]c3n2)C1] | 2575 |
| 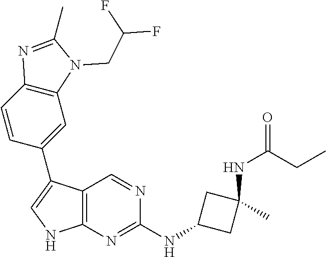 [SMILES: CCC(=O)N[C@]1(C)C[C@@H](Nc2ncc3c(-c4ccc5nc(C)n(CC(F)F)c5c4)c[nH]c3n2)C1] | 2576 |
| 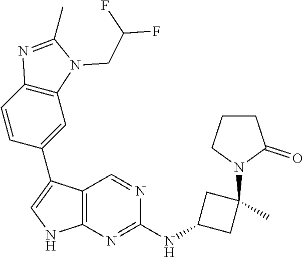 [SMILES: Cc1nc2ccc(-c3c[nH]c4nc(N[C@H]5C[C@](C)(N6CCCC6=O)C5)ncc34)cc2n1CC(F)F] | 2577 |
| 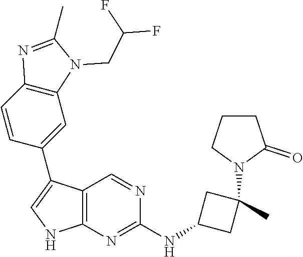 [SMILES: Cc1nc2ccc(-c3c[nH]c4nc(N[C@H]5C[C@@](C)(N6CCCC6=O)C5)ncc34)cc2n1CC(F)F] | 2578 |
| 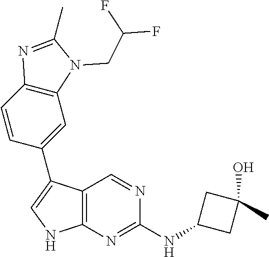 [SMILES: Cc1nc2ccc(-c3c[nH]c4nc(N[C@H]5C[C@@](C)(O)C5)ncc34)cc2n1CC(F)F] | 2579 |
| 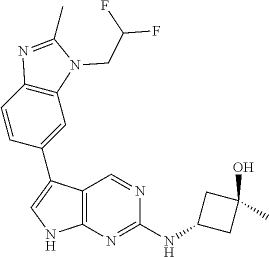 [SMILES: Cc1nc2ccc(-c3c[nH]c4nc(N[C@H]5C[C@](C)(O)C5)ncc34)cc2n1CC(F)F] | 2580 |
| 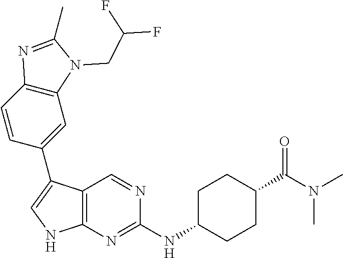 [SMILES: Cc1nc2ccc(-c3c[nH]c4nc(N[C@H]5CC[C@@H](C(=O)N(C)C)CC5)ncc34)cc2n1CC(F)F] | 2581 |
| 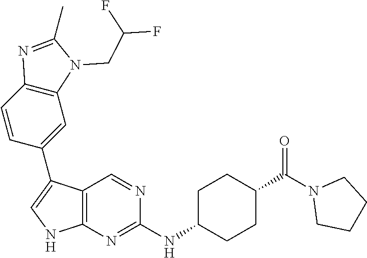 [SMILES: Cc1nc2ccc(-c3c[nH]c4nc(N[C@H]5CC[C@@H](C(=O)N6CCCC6)CC5)ncc34)cc2n1CC(F)F] | 2582 |
| 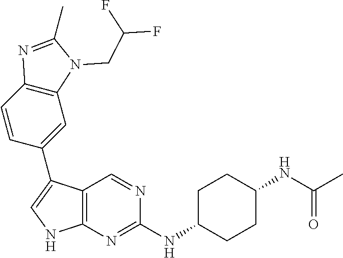 [SMILES: CC(=O)N[C@H]1CC[C@@H](Nc2ncc3c(-c4ccc5nc(C)n(CC(F)F)c5c4)c[nH]c3n2)CC1] | 2583 |
| 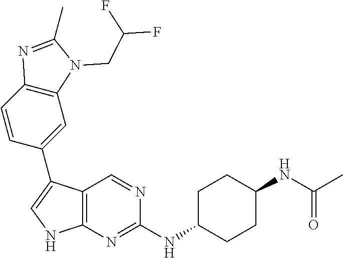 [SMILES: CC(=O)N[C@H]1CC[C@H](Nc2ncc3c(-c4ccc5nc(C)n(CC(F)F)c5c4)c[nH]c3n2)CC1] | 2584 |
| 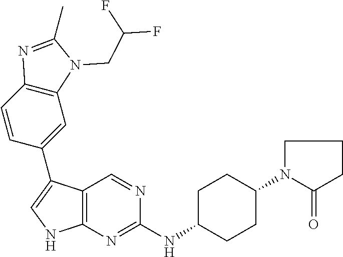 [SMILES: Cc1nc2ccc(-c3c[nH]c4nc(N[C@H]5CC[C@@H](N6CCCC6=O)CC5)ncc34)cc2n1CC(F)F] | 2585 |
| 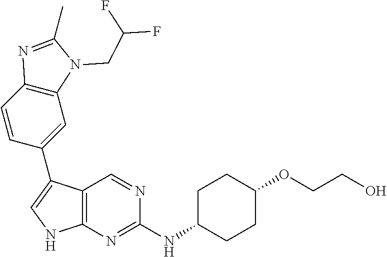 [SMILES: Cc1nc2ccc(-c3c[nH]c4nc(N[C@H]5CC[C@@H](OCCO)CC5)ncc34)cc2n1CC(F)F] | 2586 |
| 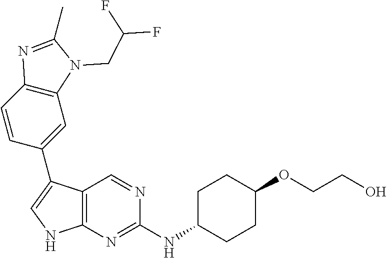 [SMILES: Cc1nc2ccc(-c3c[nH]c4nc(N[C@H]5CC[C@H](OCCO)CC5)ncc34)cc2n1CC(F)F] | 2587 |
| 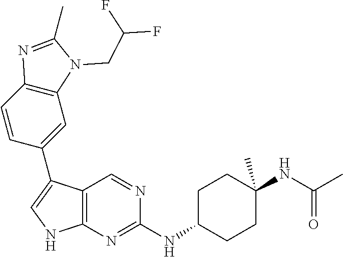 [SMILES: CC(=O)N[C@]1(C)CC[C@@H](Nc2ncc3c(-c4ccc5nc(C)n(CC(F)F)c5c4)c[nH]c3n2)CC1] | 2588 |
| 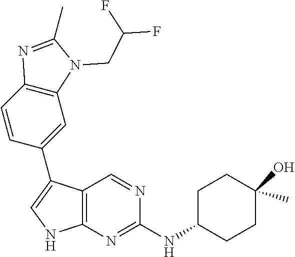 [SMILES: Cc1nc2ccc(-c3c[nH]c4nc(N[C@H]5CC[C@](C)(O)CC5)ncc34)cc2n1CC(F)F] | 2589 |
| 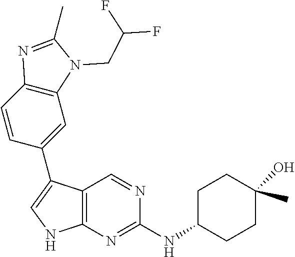 [SMILES: Cc1nc2ccc(-c3c[nH]c4nc(N[C@H]5CC[C@@](C)(O)CC5)ncc34)cc2n1CC(F)F] | 2590 |
| 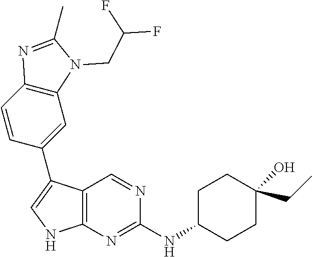 [SMILES: CC[C@]1(O)CC[C@@H](Nc2ncc3c(-c4ccc5nc(C)n(CC(F)F)c5c4)c[nH]c3n2)CC1] | 2591 |
| 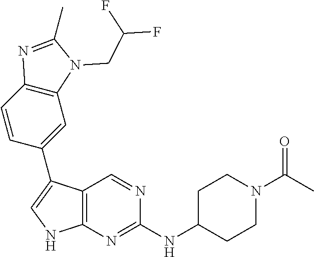 [SMILES: CC(=O)N1CCC(Nc2ncc3c(-c4ccc5nc(C)n(CC(F)F)c5c4)c[nH]c3n2)CC1] | 2592 |
| 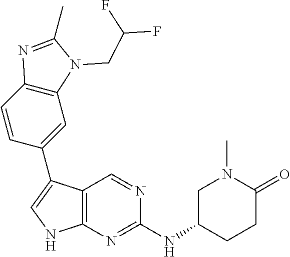 [SMILES: Cc1nc2ccc(-c3c[nH]c4nc(N[C@H]5CCC(=O)N(C)C5)ncc34)cc2n1CC(F)F] | 2593 |
| 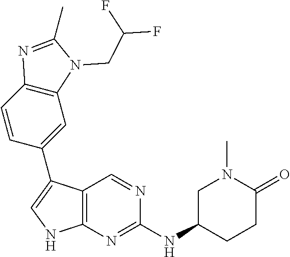 [SMILES: Cc1nc2ccc(-c3c[nH]c4nc(N[C@@H]5CCC(=O)N(C)C5)ncc34)cc2n1CC(F)F] | 2594 |
| 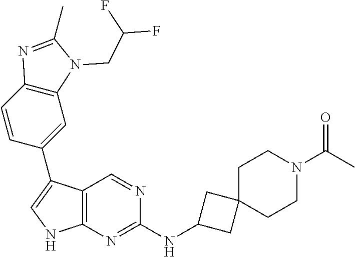 [SMILES: CC(=O)N1CCC2(CC1)CC(Nc1ncc3c(-c4ccc5nc(C)n(CC(F)F)c5c4)c[nH]c3n1)C2] | 2595 |
| 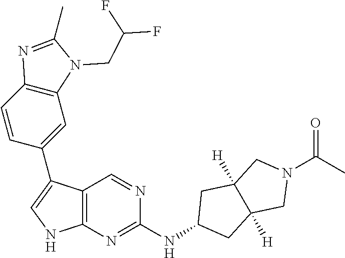 [SMILES: CC(=O)N1C[C@H]2C[C@H](Nc3ncc4c(-c5ccc6nc(C)n(CC(F)F)c6c5)c[nH]c4n3)C[C@H]2C1] | 2596 |
| 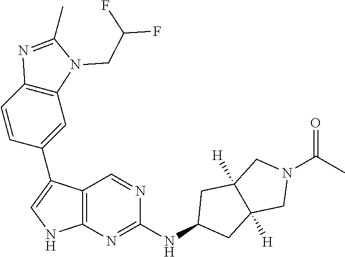 [SMILES: CC(=O)N1C[C@H]2C[C@@H](Nc3ncc4c(-c5ccc6nc(C)n(CC(F)F)c6c5)c[nH]c4n3)C[C@H]2C1] | 2597 |
| 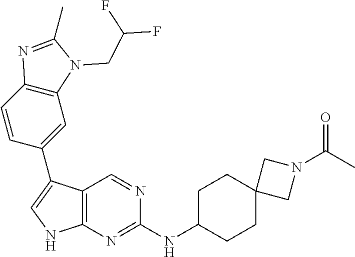 [SMILES: CC(=O)N1CC2(CCC(Nc3ncc4c(-c5ccc6nc(C)n(CC(F)F)c6c5)c[nH]c4n3)CC2)C1] | 2598 |
| 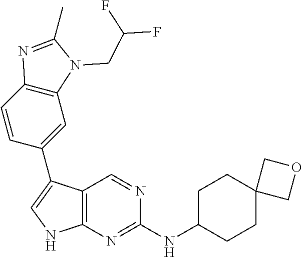 [SMILES: Cc1nc2ccc(-c3c[nH]c4nc(NC5CCC6(CC5)COC6)ncc34)cc2n1CC(F)F] | 2599 |
| 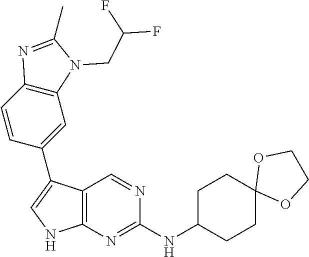 [SMILES: Cc1nc2ccc(-c3c[nH]c4nc(NC5CCC6(CC5)OCCO6)ncc34)cc2n1CC(F)F] | 2600 |
| 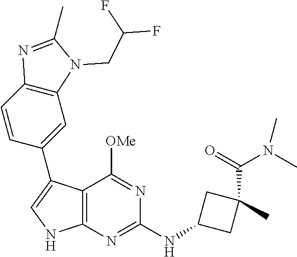 [SMILES: COc1nc(N[C@H]2C[C@@](C)(C(=O)N(C)C)C2)nc2[nH]cc(-c3ccc4nc(C)n(CC(F)F)c4c3)c12] | 2601 |
| 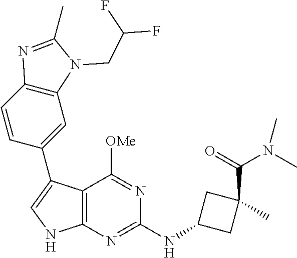 [SMILES: COc1nc(N[C@H]2C[C@](C)(C(=O)N(C)C)C2)nc2[nH]cc(-c3ccc4nc(C)n(CC(F)F)c4c3)c12] | 2602 |
| 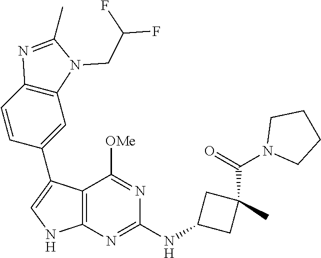 [SMILES: COc1nc(N[C@H]2C[C@@](C)(C(=O)N3CCCC3)C2)nc2[nH]cc(-c3ccc4nc(C)n(CC(F)F)c4c3)c12] | 2603 |
| 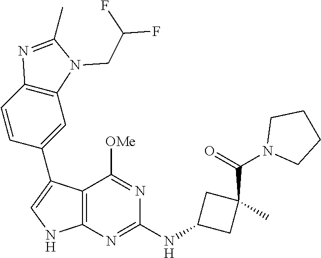 [SMILES: COc1nc(N[C@H]2C[C@](C)(C(=O)N3CCCC3)C2)nc2[nH]cc(-c3ccc4nc(C)n(CC(F)F)c4c3)c12] | 2604 |
| 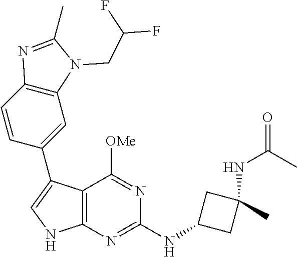 [SMILES: COc1nc(N[C@H]2C[C@@](C)(NC(C)=O)C2)nc2[nH]cc(-c3ccc4nc(C)n(CC(F)F)c4c3)c12] | 2605 |
| 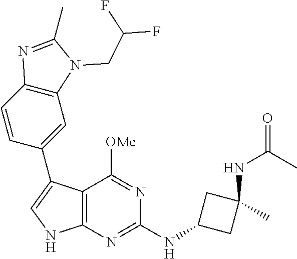 [SMILES: COc1nc(N[C@H]2C[C@](C)(NC(C)=O)C2)nc2[nH]cc(-c3ccc4nc(C)n(CC(F)F)c4c3)c12] | 2606 |
| 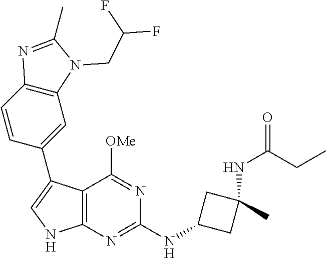 [SMILES: CCC(=O)N[C@]1(C)C[C@H](Nc2nc(OC)c3c(-c4ccc5nc(C)n(CC(F)F)c5c4)c[nH]c3n2)C1] | 2607 |
| 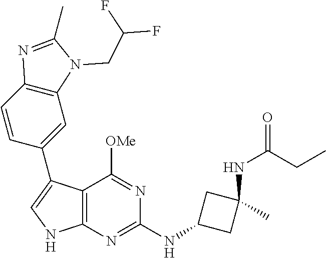 [SMILES: CCC(=O)N[C@]1(C)C[C@@H](Nc2nc(OC)c3c(-c4ccc5nc(C)n(CC(F)F)c5c4)c[nH]c3n2)C1] | 2608 |
| 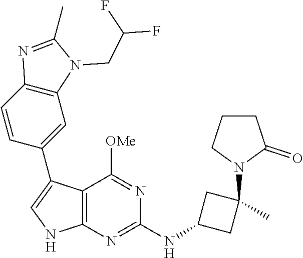 [SMILES: COc1nc(N[C@H]2C[C@](C)(N3CCCC3=O)C2)nc2[nH]cc(-c3ccc4nc(C)n(CC(F)F)c4c3)c12] | 2609 |
| 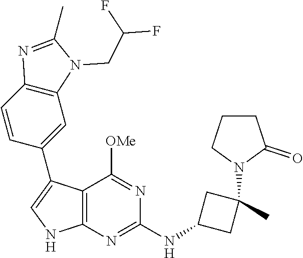 [SMILES: COc1nc(N[C@H]2C[C@@](C)(N3CCCC3=O)C2)nc2[nH]cc(-c3ccc4nc(C)n(CC(F)F)c4c3)c12] | 2610 |
| 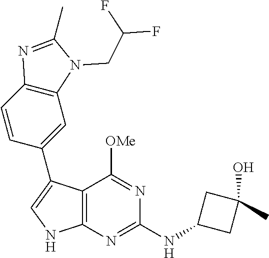 [SMILES: COc1nc(N[C@H]2C[C@@](C)(O)C2)nc2[nH]cc(-c3ccc4nc(C)n(CC(F)F)c4c3)c12] | 2611 |
| 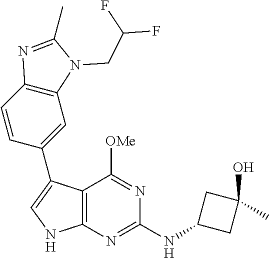 [SMILES: COc1nc(N[C@H]2C[C@](C)(O)C2)nc2[nH]cc(-c3ccc4nc(C)n(CC(F)F)c4c3)c12] | 2612 |
| 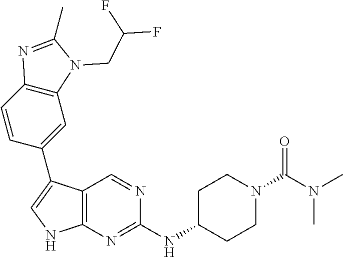 | 2613 |
| 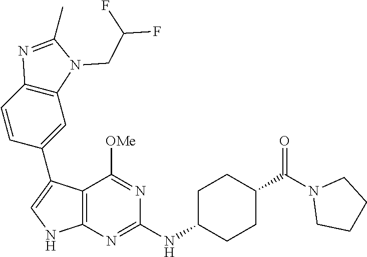 [SMILES: COc1nc(N[C@H]2CC[C@@H](C(=O)N3CCCC3)CC2)nc2[nH]cc(-c3ccc4nc(C)n(CC(F)F)c4c3)c12] | 2614 |
| 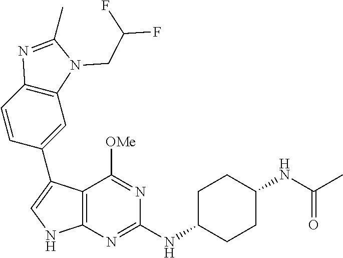 [SMILES: COc1nc(N[C@H]2CC[C@@H](NC(C)=O)CC2)nc2[nH]cc(-c3ccc4nc(C)n(CC(F)F)c4c3)c12] | 2615 |
| 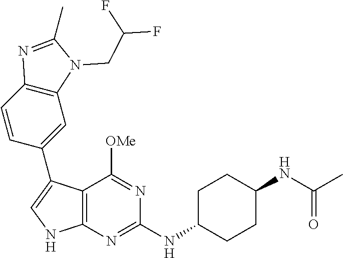 [SMILES: COc1nc(N[C@H]2CC[C@H](NC(C)=O)CC2)nc2[nH]cc(-c3ccc4nc(C)n(CC(F)F)c4c3)c12] | 2616 |
| 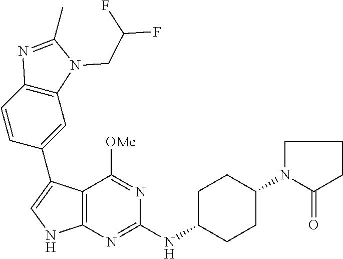 [SMILES: COc1nc(N[C@H]2CC[C@@H](N3CCCC3=O)CC2)nc2[nH]cc(-c3ccc4nc(C)n(CC(F)F)c4c3)c12] | 2617 |
| 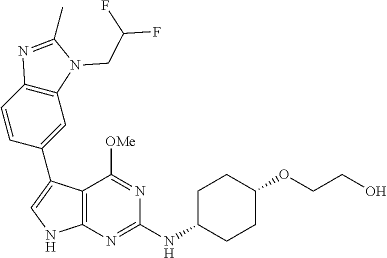 [SMILES: COc1nc(N[C@H]2CC[C@@H](OCCO)CC2)nc2[nH]cc(-c3ccc4nc(C)n(CC(F)F)c4c3)c12] | 2618 |
| 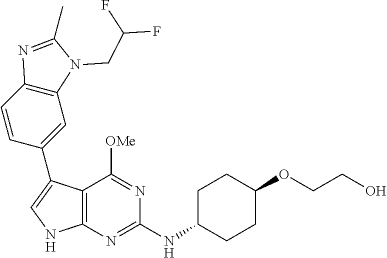 [SMILES: COc1nc(N[C@H]2CC[C@H](OCCO)CC2)nc2[nH]cc(-c3ccc4nc(C)n(CC(F)F)c4c3)c12] | 2619 |
| 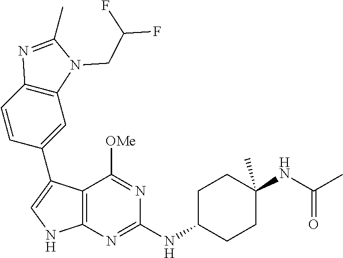 [SMILES: COc1nc(N[C@H]2CC[C@](C)(NC(C)=O)CC2)nc2[nH]cc(-c3ccc4nc(C)n(CC(F)F)c4c3)c12] | 2620 |
| 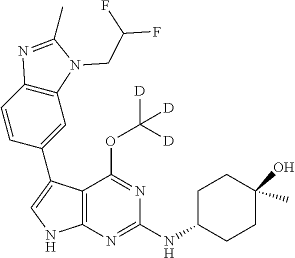 [SMILES: [2H]C([2H])([2H])Oc1nc(N[C@H]2CC[C@](C)(O)CC2)nc2[nH]cc(-c3ccc4nc(C)n(CC(F)F)c4c3)c12] | 2621 |
| 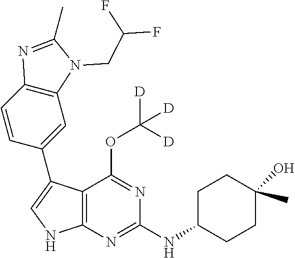 [SMILES: [2H]C([2H])([2H])Oc1nc(N[C@H]2CC[C@@](C)(O)CC2)nc2[nH]cc(-c3ccc4nc(C)n(CC(F)F)c4c3)c12] | 2622 |
| 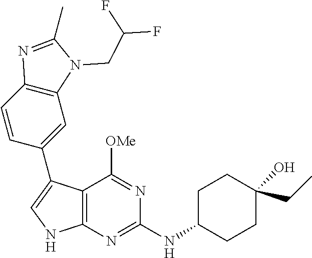 [SMILES: CC[C@]1(O)CC[C@@H](Nc2nc(OC)c3c(-c4ccc5nc(C)n(CC(F)F)c5c4)c[nH]c3n2)CC1] | 2623 |
| 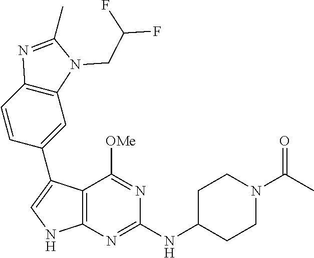 [SMILES: COc1nc(NC2CCN(C(C)=O)CC2)nc2[nH]cc(-c3ccc4nc(C)n(CC(F)F)c4c3)c12] | 2624 |
| 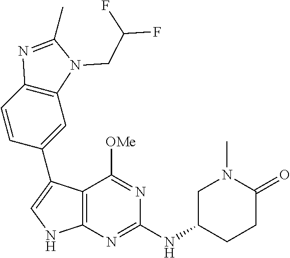 [SMILES: COc1nc(N[C@H]2CCC(=O)N(C)C2)nc2[nH]cc(-c3ccc4nc(C)n(CC(F)F)c4c3)c12] | 2625 |
| 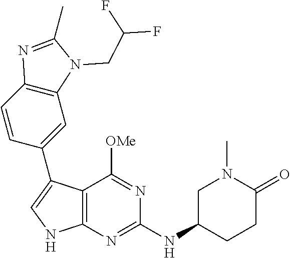 [SMILES: COc1nc(N[C@@H]2CCC(=O)N(C)C2)nc2[nH]cc(-c3ccc4nc(C)n(CC(F)F)c4c3)c12] | 2626 |
| 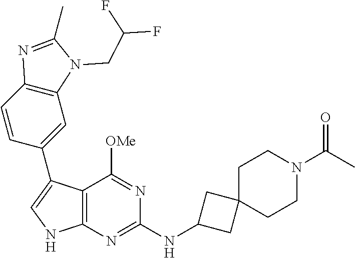 [SMILES: COc1nc(NC2CC3(CCN(C(C)=O)CC3)C2)nc2[nH]cc(-c3ccc4nc(C)n(CC(F)F)c4c3)c12] | 2627 |
| 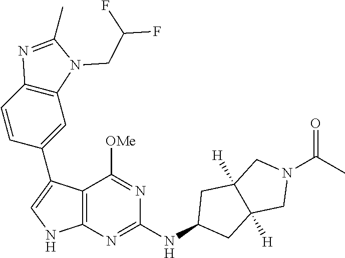 [SMILES: COc1nc(N[C@@H]2C[C@@H]3CN(C(C)=O)C[C@@H]3C2)nc2[nH]cc(-c3ccc4nc(C)n(CC(F)F)c4c3)c12] | 2628 |
| 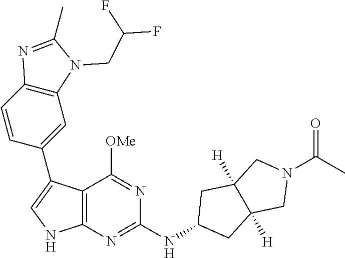 [SMILES: COc1nc(N[C@H]2C[C@@H]3CN(C(C)=O)C[C@@H]3C2)nc2[nH]cc(-c3ccc4nc(C)n(CC(F)F)c4c3)c12] | 2629 |
| 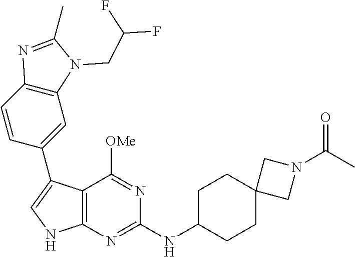 [SMILES: COc1nc(NC2CCC3(CC2)CN(C(C)=O)C3)nc2[nH]cc(-c3ccc4nc(C)n(CC(F)F)c4c3)c12] | 2630 |
| 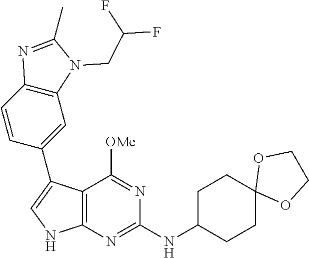 [SMILES: COc1nc(NC2CCC3(CC2)OCCO3)nc2[nH]cc(-c3ccc4nc(C)n(CC(F)F)c4c3)c12] | 2631 |
| 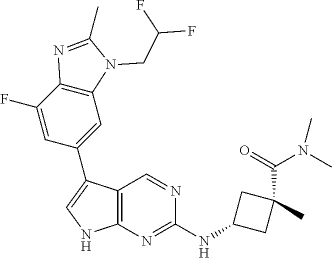 [SMILES: Cc1nc2c(F)cc(-c3c[nH]c4nc(N[C@H]5C[C@@](C)(C(=O)N(C)C)C5)ncc34)cc2n1CC(F)F] | 2632 |
| 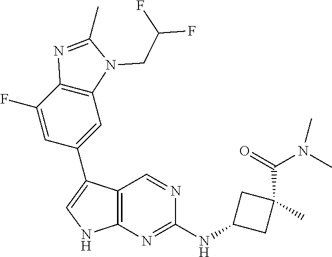 [SMILES: Cc1nc2c(F)cc(-c3c[nH]c4nc(N[C@H]5C[C@@](C)(C(=O)N(C)C)C5)ncc34)cc2n1CC(F)F] | 2633 |
| 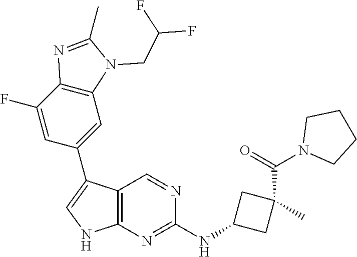 [SMILES: Cc1nc2c(F)cc(-c3c[nH]c4nc(N[C@H]5C[C@@](C)(C(=O)N6CCCC6)C5)ncc34)cc2n1CC(F)F] | 2634 |
| 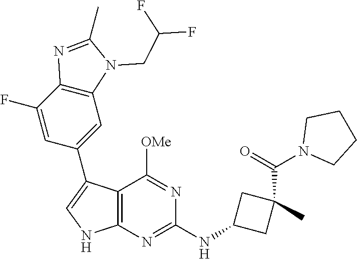 [SMILES: COc1nc(N[C@H]2C[C@@](C)(C(=O)N3CCCC3)C2)nc2[nH]cc(-c3cc(F)c4nc(C)n(CC(F)F)c4c3)c12] | 2635 |
| 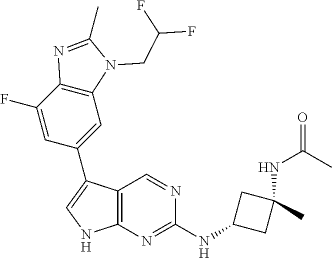 [SMILES: CC(=O)N[C@]1(C)C[C@H](Nc2ncc3c(-c4cc(F)c5nc(C)n(CC(F)F)c5c4)c[nH]c3n2)C1] | 2636 |
| 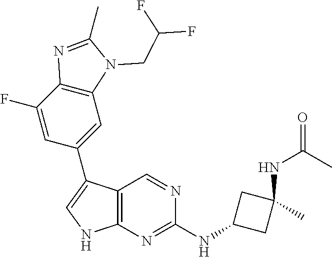 [SMILES: CC(=O)N[C@]1(C)C[C@@H](Nc2ncc3c(-c4cc(F)c5nc(C)n(CC(F)F)c5c4)c[nH]c3n2)C1] | 2637 |
| 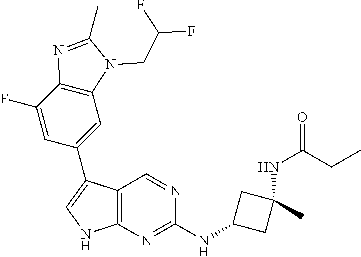 [SMILES: CCC(=O)N[C@]1(C)C[C@H](Nc2ncc3c(-c4cc(F)c5nc(C)n(CC(F)F)c5c4)c[nH]c3n2)C1] | 2638 |
| 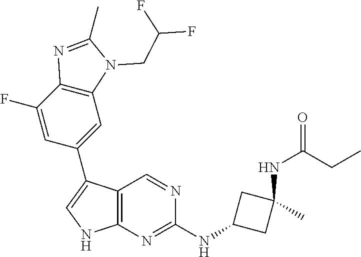 [SMILES: CCC(=O)N[C@]1(C)C[C@@H](Nc2ncc3c(-c4cc(F)c5nc(C)n(CC(F)F)c5c4)c[nH]c3n2)C1] | 2639 |
| 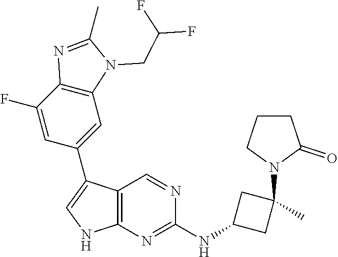 [SMILES: Cc1nc2c(F)cc(-c3c[nH]c4nc(N[C@H]5C[C@](C)(N6CCCC6=O)C5)ncc34)cc2n1CC(F)F] | 2640 |
| 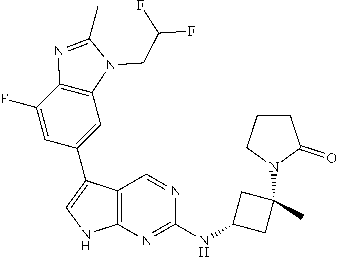 [SMILES: Cc1nc2c(F)cc(-c3c[nH]c4nc(N[C@H]5C[C@@](C)(N6CCCC6=O)C5)ncc34)cc2n1CC(F)F] | 2641 |
| 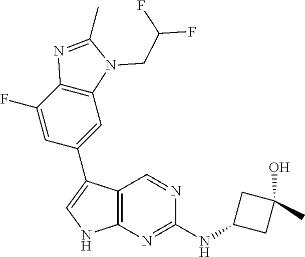 [SMILES: Cc1nc2c(F)cc(-c3c[nH]c4nc(N[C@H]5C[C@@](C)(O)C5)ncc34)cc2n1CC(F)F] | 2642 |
| 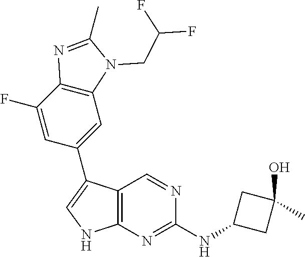 [SMILES: Cc1nc2c(F)cc(-c3c[nH]c4nc(N[C@H]5C[C@](C)(O)C5)ncc34)cc2n1CC(F)F] | 2643 |
| 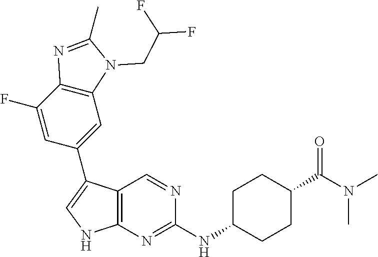 [SMILES: Cc1nc2c(F)cc(-c3c[nH]c4nc(N[C@H]5CC[C@@H](C(=O)N(C)C)CC5)ncc34)cc2n1CC(F)F] | 2644 |
| 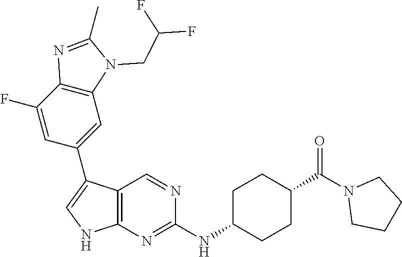 [SMILES: Cc1nc2c(F)cc(-c3c[nH]c4nc(N[C@H]5CC[C@@H](C(=O)N6CCCC6)CC5)ncc34)cc2n1CC(F)F] | 2645 |
| 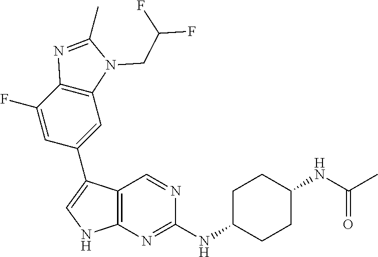 [SMILES: CC(=O)N[C@H]1CC[C@@H](Nc2ncc3c(-c4cc(F)c5nc(C)n(CC(F)F)c5c4)c[nH]c3n2)CC1] | 2646 |
| 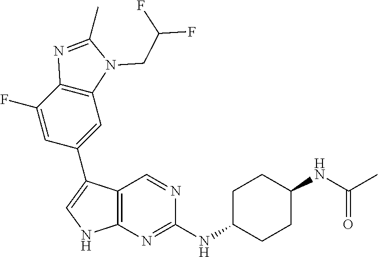 [SMILES: CC(=O)N[C@H]1CC[C@H](Nc2ncc3c(-c4cc(F)c5nc(C)n(CC(F)F)c5c4)c[nH]c3n2)CC1] | 2647 |
| 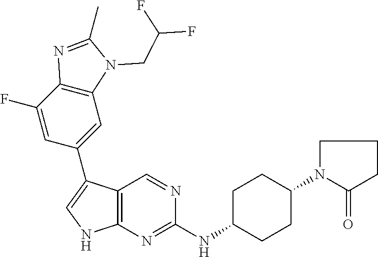 [SMILES: Cc1nc2c(F)cc(-c3c[nH]c4nc(N[C@H]5CC[C@@H](N6CCCC6=O)CC5)ncc34)cc2n1CC(F)F] | 2648 |
| 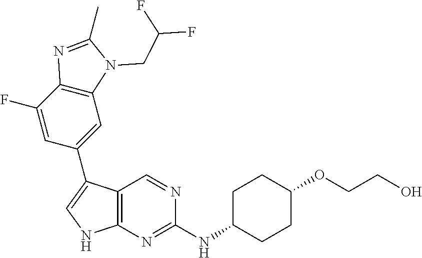 [SMILES: Cc1nc2c(F)cc(-c3c[nH]c4nc(N[C@H]5CC[C@@H](OCCO)CC5)ncc34)cc2n1CC(F)F] | 2649 |
| 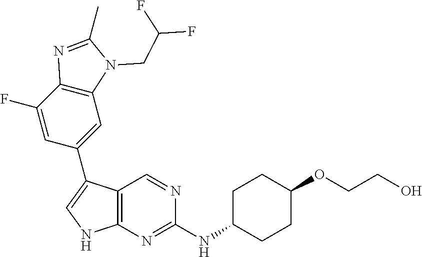 [SMILES: Cc1nc2c(F)cc(-c3c[nH]c4nc(N[C@H]5CC[C@H](OCCO)CC5)ncc34)cc2n1CC(F)F] | 2650 |
| 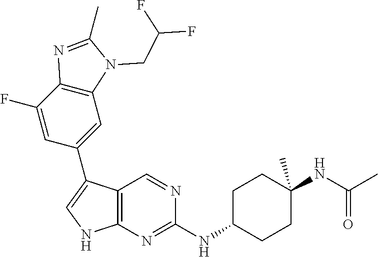 [SMILES: CC(=O)N[C@]1(C)CC[C@@H](Nc2ncc3c(-c4cc(F)c5nc(C)n(CC(F)F)c5c4)c[nH]c3n2)CC1] | 2651 |
| 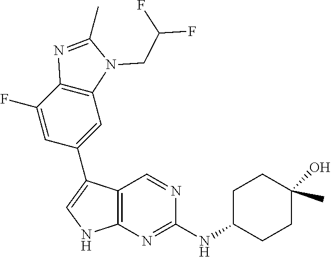 [SMILES: Cc1nc2c(F)cc(-c3c[nH]c4nc(N[C@H]5CC[C@@](C)(O)CC5)ncc34)cc2n1CC(F)F] | 2652 |
| 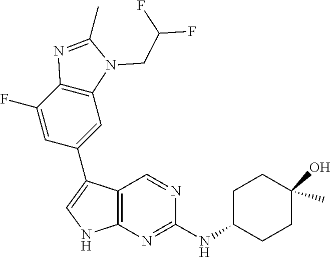 [SMILES: Cc1nc2c(F)cc(-c3c[nH]c4nc(N[C@H]5CC[C@](C)(O)CC5)ncc34)cc2n1CC(F)F] | 2653 |
| 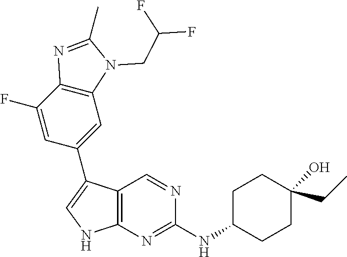 [SMILES: CC[C@]1(O)CC[C@@H](Nc2ncc3c(-c4cc(F)c5nc(C)n(CC(F)F)c5c4)c[nH]c3n2)CC1] | 2654 |
| 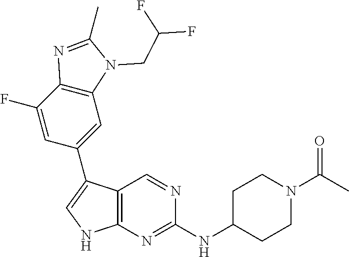 [SMILES: CC(=O)N1CCC(Nc2ncc3c(-c4cc(F)c5nc(C)n(CC(F)F)c5c4)c[nH]c3n2)CC1] | 2655 |
| 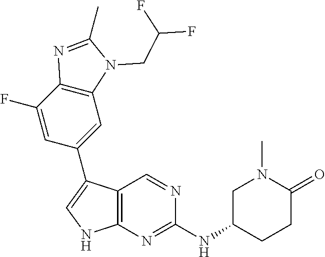 [SMILES: Cc1nc2c(F)cc(-c3c[nH]c4nc(N[C@H]5CCC(=O)N(C)C5)ncc34)cc2n1CC(F)F] | 2656 |
| 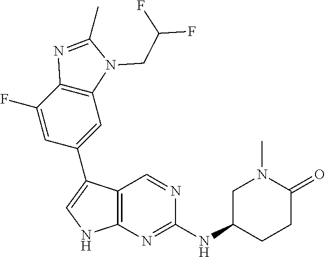 [SMILES: Cc1nc2c(F)cc(-c3c[nH]c4nc(N[C@@H]5CCC(=O)N(C)C5)ncc34)cc2n1CC(F)F] | 2657 |
| 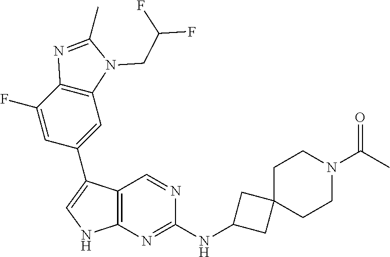 [SMILES: CC(=O)N1CCC2(CC1)CC(Nc1ncc3c(-c4cc(F)c5nc(C)n(CC(F)F)c5c4)c[nH]c3n1)C2] | 2658 |
| 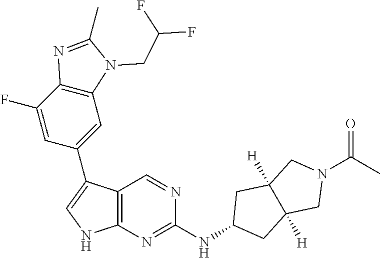 [SMILES: CC(=O)N1C[C@H]2C[C@H](Nc3ncc4c(-c5cc(F)c6nc(C)n(CC(F)F)c6c5)c[nH]c4n3)C[C@H]2C1] | 2659 |
| 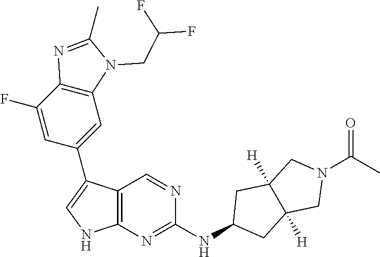 [SMILES: CC(=O)N1C[C@H]2C[C@@H](Nc3ncc4c(-c5cc(F)c6nc(C)n(CC(F)F)c6c5)c[nH]c4n3)C[C@H]2C1] | 2660 |
| 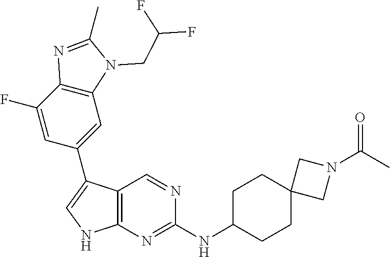 [SMILES: CC(=O)N1CC2(CCC(Nc3ncc4c(-c5cc(F)c6nc(C)n(CC(F)F)c6c5)c[nH]c4n3)CC2)C1] | 2661 |
| 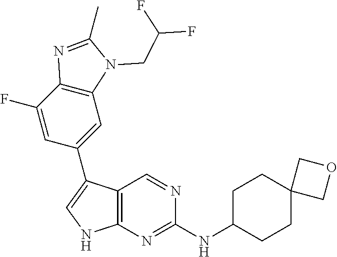 [SMILES: Cc1nc2c(F)cc(-c3c[nH]c4nc(NC5CCC6(CC5)COC6)ncc34)cc2n1CC(F)F] | 2662 |
| 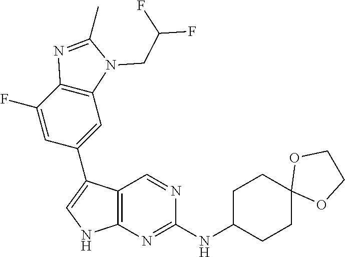 [SMILES: Cc1nc2c(F)cc(-c3c[nH]c4nc(NC5CCC6(CC5)OCCO6)ncc34)cc2n1CC(F)F] | 2663 |
| 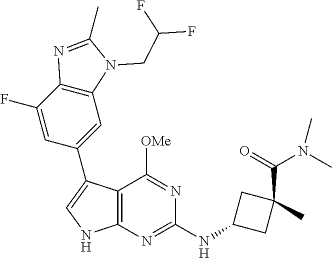 [SMILES: COc1nc(N[C@H]2C[C@](C)(C(=O)N(C)C)C2)nc2[nH]cc(-c3cc(F)c4nc(C)n(CC(F)F)c4c3)c12] | 2664 |
| 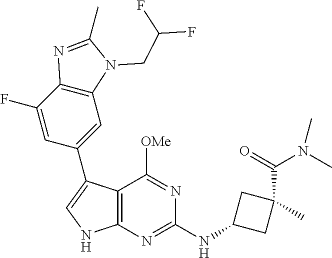 [SMILES: COc1nc(N[C@H]2C[C@@](C)(C(=O)N(C)C)C2)nc2[nH]cc(-c3cc(F)c4nc(C)n(CC(F)F)c4c3)c12] | 2665 |
| 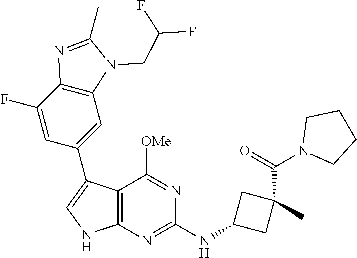 [SMILES: COc1nc(N[C@H]2C[C@@](C)(C(=O)N3CCCC3)C2)nc2[nH]cc(-c3cc(F)c4nc(C)n(CC(F)F)c4c3)c12] | 2666 |
| 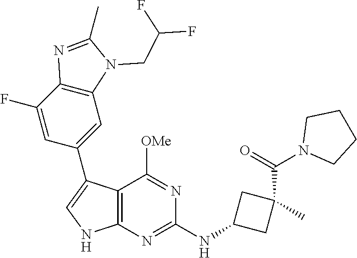 [SMILES: COc1nc(N[C@H]2C[C@@](C)(C(=O)N3CCCC3)C2)nc2[nH]cc(-c3cc(F)c4nc(C)n(CC(F)F)c4c3)c12] | 2667 |
| 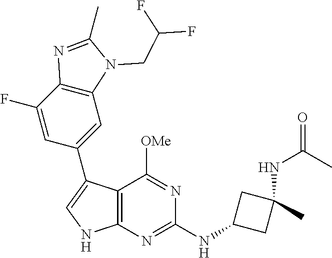 [SMILES: COc1nc(N[C@H]2C[C@@](C)(NC(C)=O)C2)nc2[nH]cc(-c3cc(F)c4nc(C)n(CC(F)F)c4c3)c12] | 2668 |
| 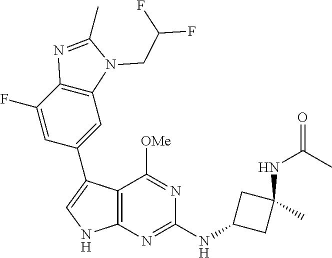 [SMILES: COc1nc(N[C@H]2C[C@](C)(NC(C)=O)C2)nc2[nH]cc(-c3cc(F)c4nc(C)n(CC(F)F)c4c3)c12] | 2669 |
| 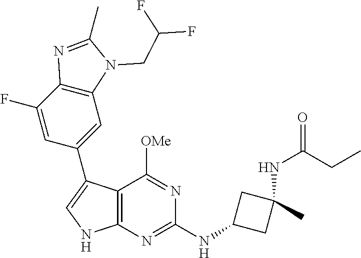 [SMILES: CCC(=O)N[C@]1(C)C[C@H](Nc2nc(OC)c3c(-c4cc(F)c5nc(C)n(CC(F)F)c5c4)c[nH]c3n2)C1] | 2670 |
| 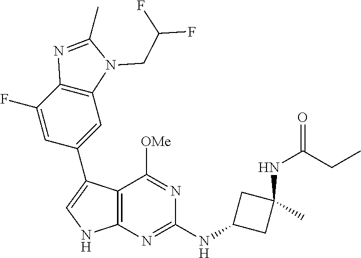 [SMILES: CCC(=O)N[C@]1(C)C[C@@H](Nc2nc(OC)c3c(-c4cc(F)c5nc(C)n(CC(F)F)c5c4)c[nH]c3n2)C1] | 2671 |
| 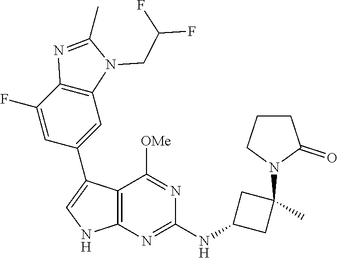 [SMILES: COc1nc(N[C@H]2C[C@](C)(N3CCCC3=O)C2)nc2[nH]cc(-c3cc(F)c4nc(C)n(CC(F)F)c4c3)c12] | 2672 |
| 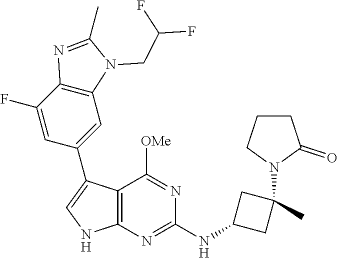 [SMILES: COc1nc(N[C@H]2C[C@@](C)(N3CCCC3=O)C2)nc2[nH]cc(-c3cc(F)c4nc(C)n(CC(F)F)c4c3)c12] | 2673 |
| 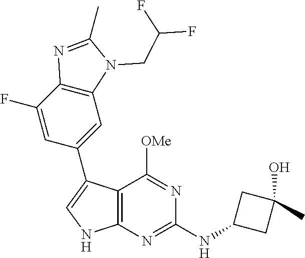 [SMILES: COc1nc(N[C@H]2C[C@@](C)(O)C2)nc2[nH]cc(-c3cc(F)c4nc(C)n(CC(F)F)c4c3)c12] | 2674 |
| 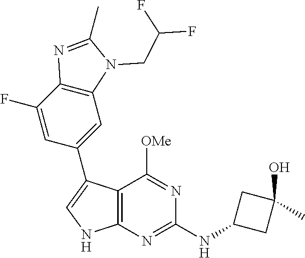 [SMILES: COc1nc(N[C@H]2C[C@](C)(O)C2)nc2[nH]cc(-c3cc(F)c4nc(C)n(CC(F)F)c4c3)c12] | 2675 |
| 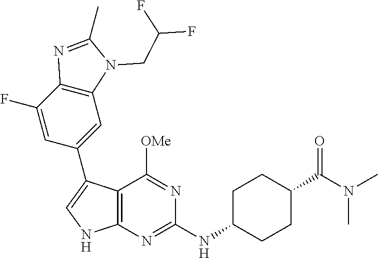 [SMILES: COc1nc(N[C@H]2CC[C@@H](C(=O)N(C)C)CC2)nc2[nH]cc(-c3cc(F)c4nc(C)n(CC(F)F)c4c3)c12] | 2676 |
| 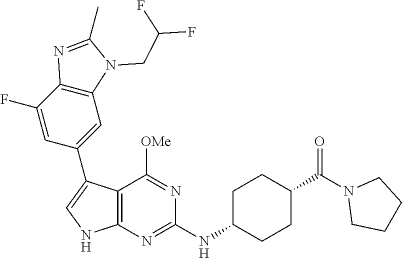 [SMILES: COc1nc(N[C@H]2CC[C@@H](C(=O)N3CCCC3)CC2)nc2[nH]cc(-c3cc(F)c4nc(C)n(CC(F)F)c4c3)c12] | 2677 |
| 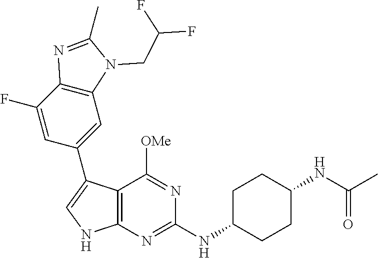 [SMILES: COc1nc(N[C@H]2CC[C@@H](NC(C)=O)CC2)nc2[nH]cc(-c3cc(F)c4nc(C)n(CC(F)F)c4c3)c12] | 2678 |
| 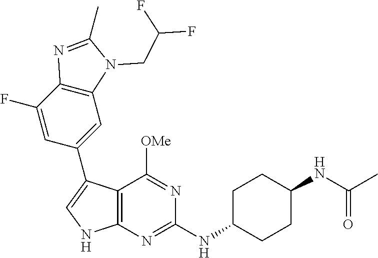 [SMILES: COc1nc(N[C@H]2CC[C@H](NC(C)=O)CC2)nc2[nH]cc(-c3cc(F)c4nc(C)n(CC(F)F)c4c3)c12] | 2679 |
| 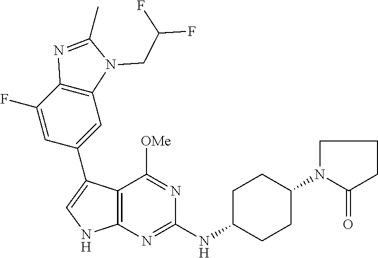 [SMILES: COc1nc(N[C@H]2CC[C@@H](N3CCCC3=O)CC2)nc2[nH]cc(-c3cc(F)c4nc(C)n(CC(F)F)c4c3)c12] | 2680 |
| 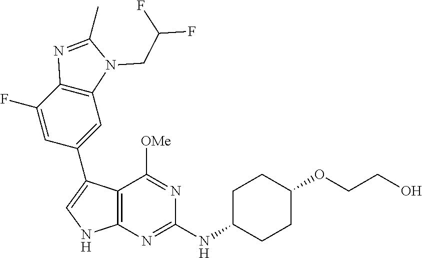 [SMILES: COc1nc(N[C@H]2CC[C@@H](OCCO)CC2)nc2[nH]cc(-c3cc(F)c4nc(C)n(CC(F)F)c4c3)c12] | 2681 |
| 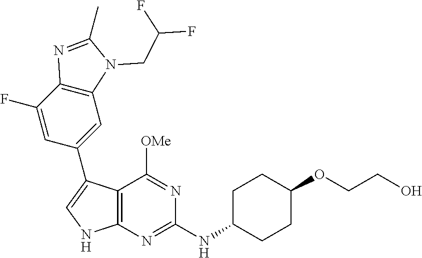 [SMILES: COc1nc(N[C@H]2CC[C@H](OCCO)CC2)nc2[nH]cc(-c3cc(F)c4nc(C)n(CC(F)F)c4c3)c12] | 2682 |
| 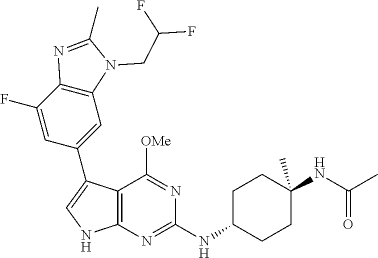 [SMILES: COc1nc(N[C@H]2CC[C@](C)(NC(C)=O)CC2)nc2[nH]cc(-c3cc(F)c4nc(C)n(CC(F)F)c4c3)c12] | 2683 |
| 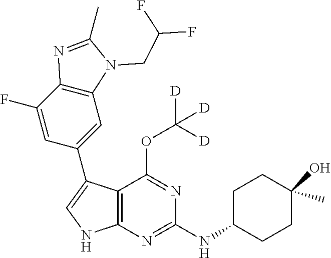 [SMILES: [2H]C([2H])([2H])Oc1nc(N[C@H]2CC[C@](C)(O)CC2)nc2[nH]cc(-c3cc(F)c4nc(C)n(CC(F)F)c4c3)c12] | 2684 |
| 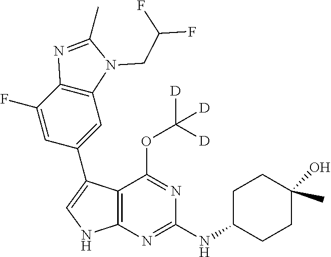 [SMILES: [2H]C([2H])([2H])Oc1nc(N[C@H]2CC[C@@](C)(O)CC2)nc2[nH]cc(-c3cc(F)c4nc(C)n(CC(F)F)c4c3)c12] | 2685 |
| 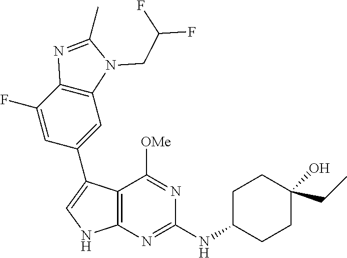 [SMILES: CC[C@]1(O)CC[C@@H](Nc2nc(OC)c3c(-c4cc(F)c5nc(C)n(CC(F)F)c5c4)c[nH]c3n2)CC1] | 2686 |
| 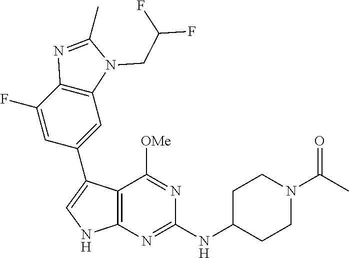 [SMILES: COc1nc(NC2CCN(C(C)=O)CC2)nc2[nH]cc(-c3cc(F)c4nc(C)n(CC(F)F)c4c3)c12] | 2687 |
| 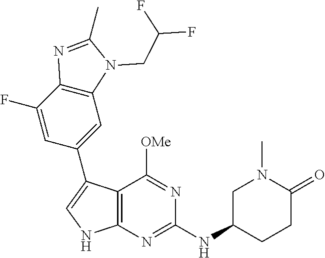 [SMILES: COc1nc(N[C@@H]2CCC(=O)N(C)C2)nc2[nH]cc(-c3cc(F)c4nc(C)n(CC(F)F)c4c3)c12] | 2688 |
| 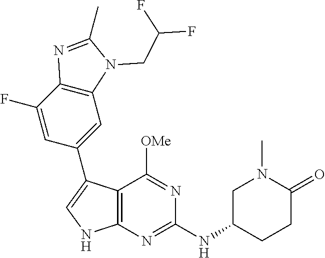 [SMILES: COc1nc(N[C@H]2CCC(=O)N(C)C2)nc2[nH]cc(-c3cc(F)c4nc(C)n(CC(F)F)c4c3)c12] | 2689 |
| 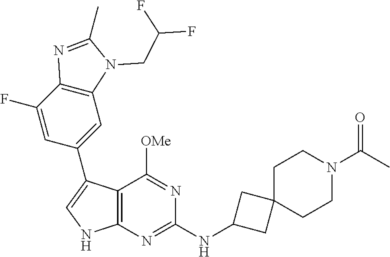 [SMILES: COc1nc(NC2CC3(CCN(C(C)=O)CC3)C2)nc2[nH]cc(-c3cc(F)c4nc(C)n(CC(F)F)c4c3)c12] | 2690 |
| 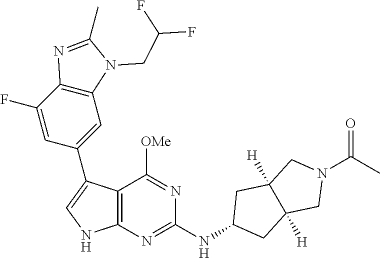 [SMILES: COc1nc(N[C@H]2C[C@@H]3CN(C(C)=O)C[C@@H]3C2)nc2[nH]cc(-c3cc(F)c4nc(C)n(CC(F)F)c4c3)c12] | 2691 |
| 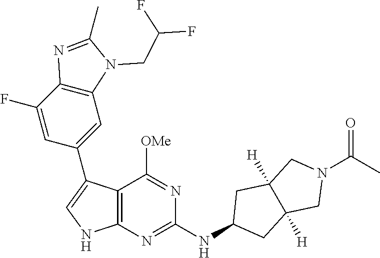 [SMILES: COc1nc(N[C@@H]2C[C@@H]3CN(C(C)=O)C[C@@H]3C2)nc2[nH]cc(-c3cc(F)c4nc(C)n(CC(F)F)c4c3)c12] | 2692 |
| 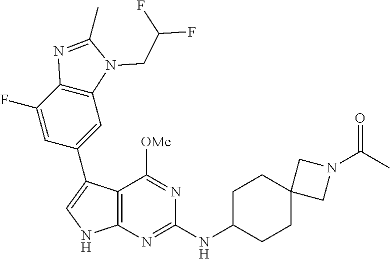 [SMILES: COc1nc(NC2CCC3(CC2)CN(C(C)=O)C3)nc2[nH]cc(-c3cc(F)c4nc(C)n(CC(F)F)c4c3)c12] | 2693 |
| 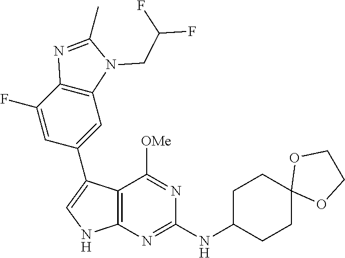 [SMILES: COc1nc(NC2CCC3(CC2)OCCO3)nc2[nH]cc(-c3cc(F)c4nc(C)n(CC(F)F)c4c3)c12] | 2694 |
| 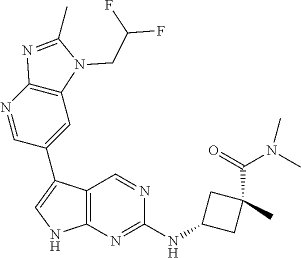 [SMILES: Cc1nc2ncc(-c3c[nH]c4nc(N[C@H]5C[C@@](C)(C(=O)N(C)C)C5)ncc34)cc2n1CC(F)F] | 2695 |
| 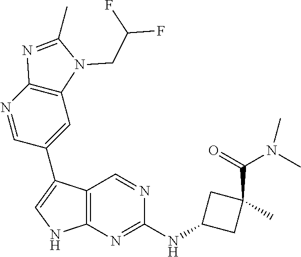 [SMILES: Cc1nc2ncc(-c3c[nH]c4nc(N[C@H]5C[C@](C)(C(=O)N(C)C)C5)ncc34)cc2n1CC(F)F] | 2696 |
| 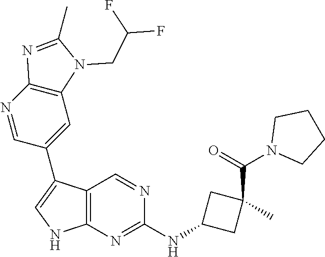 [SMILES: Cc1nc2ncc(-c3c[nH]c4nc(N[C@H]5C[C@](C)(C(=O)N6CCCC6)C5)ncc34)cc2n1CC(F)F] | 2697 |
| 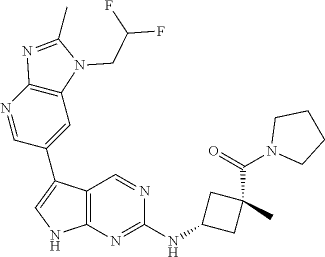 [SMILES: Cc1nc2ncc(-c3c[nH]c4nc(N[C@H]5C[C@@](C)(C(=O)N6CCCC6)C5)ncc34)cc2n1CC(F)F] | 2698 |
| 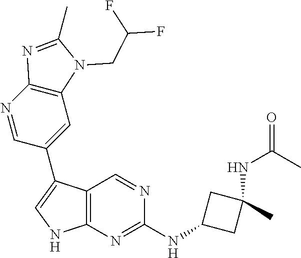 [SMILES: CC(=O)N[C@]1(C)C[C@H](Nc2ncc3c(-c4cnc5nc(C)n(CC(F)F)c5c4)c[nH]c3n2)C1] | 2999 |
| 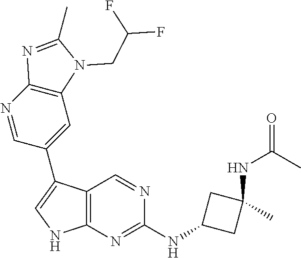 [SMILES: CC(=O)N[C@]1(C)C[C@@H](Nc2ncc3c(-c4cnc5nc(C)n(CC(F)F)c5c4)c[nH]c3n2)C1] | 2700 |
| 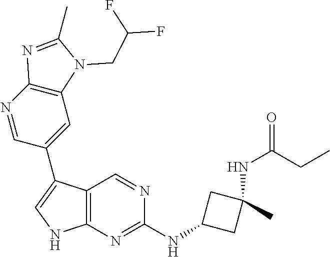 [SMILES: CCC(=O)N[C@]1(C)C[C@H](Nc2ncc3c(-c4cnc5nc(C)n(CC(F)F)c5c4)c[nH]c3n2)C1] | 2701 |
| 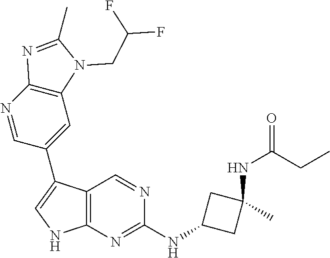 [SMILES: CCC(=O)N[C@]1(C)C[C@@H](Nc2ncc3c(-c4cnc5nc(C)n(CC(F)F)c5c4)c[nH]c3n2)C1] | 2702 |
| 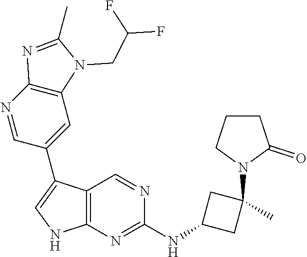 [SMILES: Cc1nc2ncc(-c3c[nH]c4nc(N[C@H]5C[C@](C)(N6CCCC6=O)C5)ncc34)cc2n1CC(F)F] | 2703 |
| 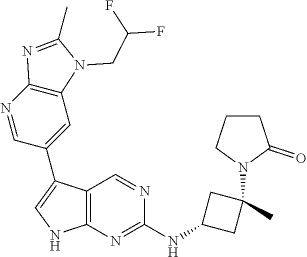 [SMILES: Cc1nc2ncc(-c3c[nH]c4nc(N[C@H]5C[C@@](C)(N6CCCC6=O)C5)ncc34)cc2n1CC(F)F] | 2704 |
| 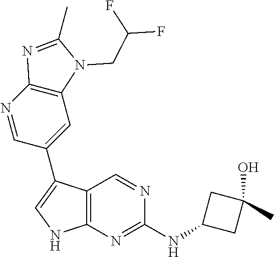 [SMILES: Cc1nc2ncc(-c3c[nH]c4nc(N[C@H]5C[C@@](C)(O)C5)ncc34)cc2n1CC(F)F] | 2705 |
| 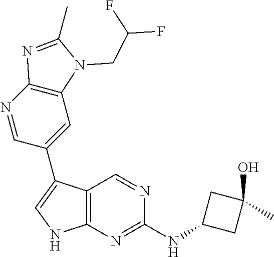 [SMILES: Cc1nc2ncc(-c3c[nH]c4nc(N[C@H]5C[C@](C)(O)C5)ncc34)cc2n1CC(F)F] | 2706 |
| 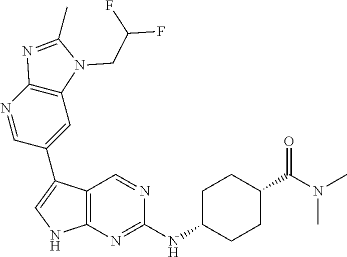 [SMILES: Cc1nc2ncc(-c3c[nH]c4nc(N[C@H]5CC[C@@H](C(=O)N(C)C)CC5)ncc34)cc2n1CC(F)F] | 2707 |
| 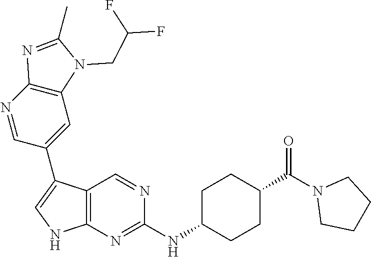 [SMILES: Cc1nc2ncc(-c3c[nH]c4nc(N[C@H]5CC[C@@H](C(=O)N6CCCC6)CC5)ncc34)cc2n1CC(F)F] | 2708 |
| 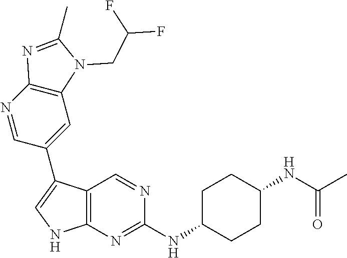 [SMILES: CC(=O)N[C@H]1CC[C@@H](Nc2ncc3c(-c4cnc5nc(C)n(CC(F)F)c5c4)c[nH]c3n2)CC1] | 2709 |
| 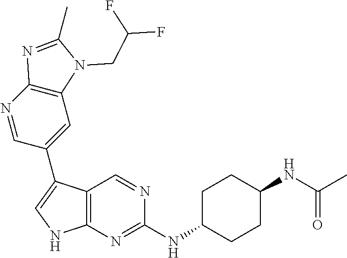 [SMILES: CC(=O)N[C@H]1CC[C@H](Nc2ncc3c(-c4cnc5nc(C)n(CC(F)F)c5c4)c[nH]c3n2)CC1] | 2710 |
| 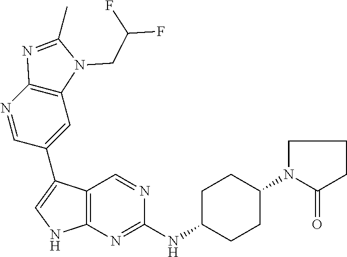 [SMILES: Cc1nc2ncc(-c3c[nH]c4nc(N[C@H]5CC[C@@H](N6CCCC6=O)CC5)ncc34)cc2n1CC(F)F] | 2711 |
| 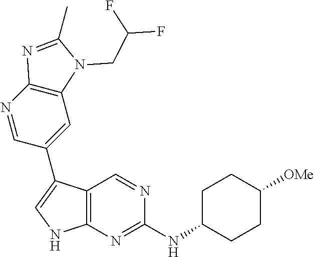 [SMILES: CO[C@H]1CC[C@@H](Nc2ncc3c(-c4cnc5nc(C)n(CC(F)F)c5c4)c[nH]c3n2)CC1] | 2712 |
| 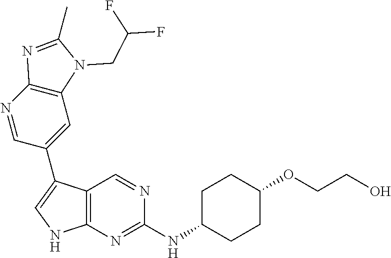 [SMILES: Cc1nc2ncc(-c3c[nH]c4nc(N[C@H]5CC[C@@H](OCCO)CC5)ncc34)cc2n1CC(F)F] | 2713 |
| 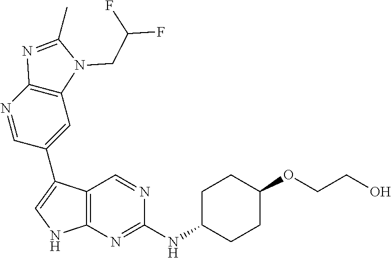 [SMILES: Cc1nc2ncc(-c3c[nH]c4nc(N[C@H]5CC[C@H](OCCO)CC5)ncc34)cc2n1CC(F)F] | 2714 |
| 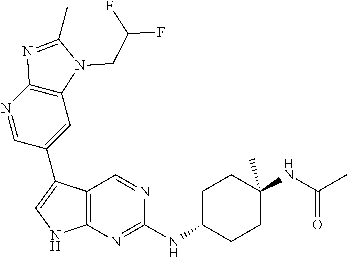 [SMILES: CC(=O)N[C@]1(C)CC[C@@H](Nc2ncc3c(-c4cnc5nc(C)n(CC(F)F)c5c4)c[nH]c3n2)CC1] | 2715 |
| 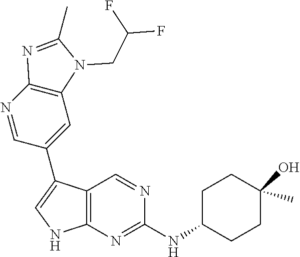 [SMILES: Cc1nc2ncc(-c3c[nH]c4nc(N[C@H]5CC[C@](C)(O)CC5)ncc34)cc2n1CC(F)F] | 2716 |
| 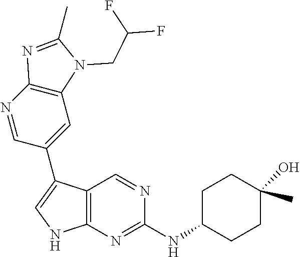 [SMILES: Cc1nc2ncc(-c3c[nH]c4nc(N[C@H]5CC[C@@](C)(O)CC5)ncc34)cc2n1CC(F)F] | 2717 |
| 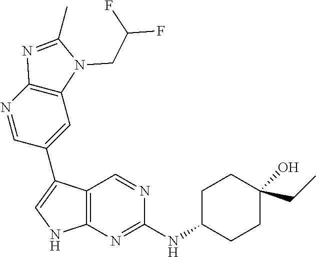 [SMILES: CC[C@]1(O)CC[C@@H](Nc2ncc3c(-c4cnc5nc(C)n(CC(F)F)c5c4)c[nH]c3n2)CC1] | 2718 |
| 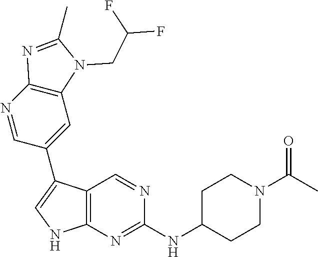 [SMILES: CC(=O)N1CCC(Nc2ncc3c(-c4cnc5nc(C)n(CC(F)F)c5c4)c[nH]c3n2)CC1] | 2719 |
| 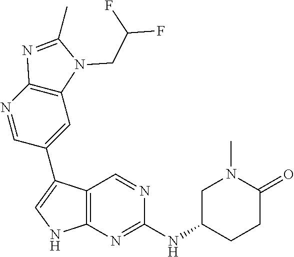 [SMILES: Cc1nc2ncc(-c3c[nH]c4nc(N[C@H]5CCC(=O)N(C)C5)ncc34)cc2n1CC(F)F] | 2720 |
| 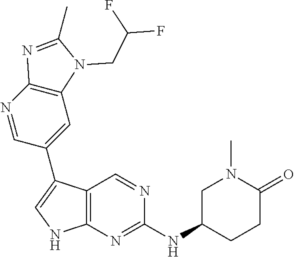 [SMILES: Cc1nc2ncc(-c3c[nH]c4nc(N[C@@H]5CCC(=O)N(C)C5)ncc34)cc2n1CC(F)F] | 2721 |
| 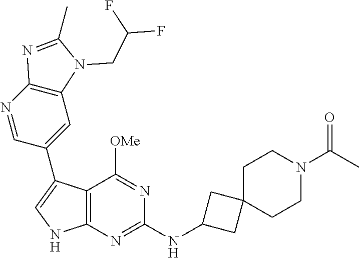 [SMILES: COc1nc(NC2CC3(CCN(C(C)=O)CC3)C2)nc2[nH]cc(-c3cnc4nc(C)n(CC(F)F)c4c3)c12] | 2722 |
| 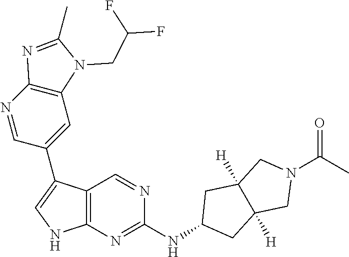 [SMILES: CC(=O)N1C[C@H]2C[C@H](Nc3ncc4c(-c5cnc6nc(C)n(CC(F)F)c6c5)c[nH]c4n3)C[C@H]2C1] | 2723 |
| 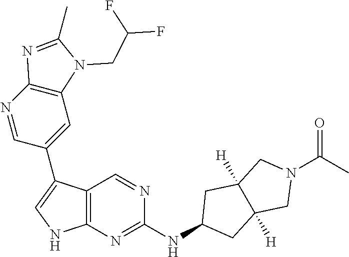 [SMILES: CC(=O)N1C[C@H]2C[C@@H](Nc3ncc4c(-c5cnc6nc(C)n(CC(F)F)c6c5)c[nH]c4n3)C[C@H]2C1] | 2724 |
| 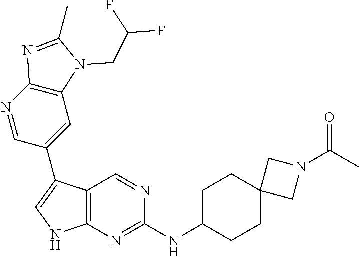 [SMILES: CC(=O)N1CC2(CCC(Nc3ncc4c(-c5cnc6nc(C)n(CC(F)F)c6c5)c[nH]c4n3)CC2)C1] | 2725 |
| 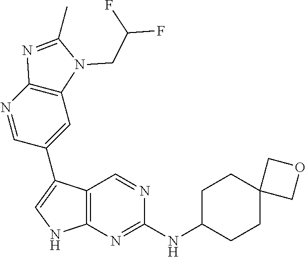 [SMILES: Cc1nc2ncc(-c3c[nH]c4nc(NC5CCC6(CC5)COC6)ncc34)cc2n1CC(F)F] | 2726 |
| 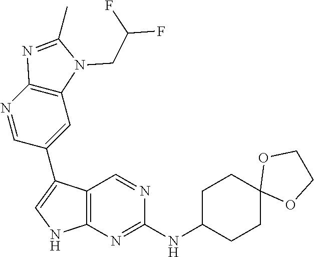 [SMILES: Cc1nc2ncc(-c3c[nH]c4nc(NC5CCC6(CC5)OCCO6)ncc34)cc2n1CC(F)F] | 2727 |
| 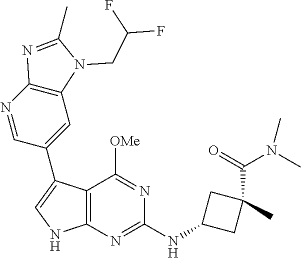 [SMILES: COc1nc(N[C@H]2C[C@@](C)(C(=O)N(C)C)C2)nc2[nH]cc(-c3cnc4nc(C)n(CC(F)F)c4c3)c12] | 2728 |
| 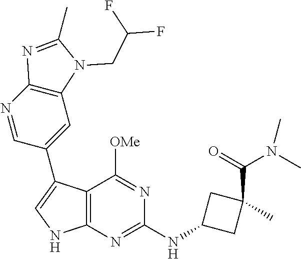 [SMILES: COc1nc(N[C@H]2C[C@](C)(C(=O)N(C)C)C2)nc2[nH]cc(-c3cnc4nc(C)n(CC(F)F)c4c3)c12] | 2729 |
| 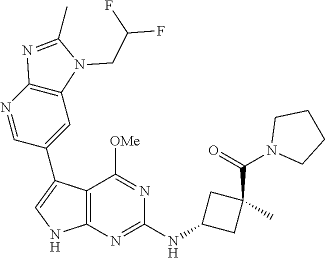 [SMILES: COc1nc(N[C@H]2C[C@](C)(C(=O)N3CCCC3)C2)nc2[nH]cc(-c3cnc4nc(C)n(CC(F)F)c4c3)c12] | 2730 |
| 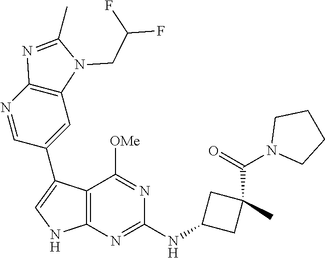 [SMILES: COc1nc(N[C@H]2C[C@@](C)(C(=O)N3CCCC3)C2)nc2[nH]cc(-c3cnc4nc(C)n(CC(F)F)c4c3)c12] | 2731 |
| 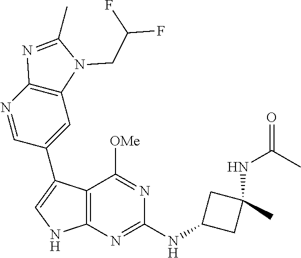 [SMILES: COc1nc(N[C@H]2C[C@@](C)(NC(C)=O)C2)nc2[nH]cc(-c3cnc4nc(C)n(CC(F)F)c4c3)c12] | 2732 |
| 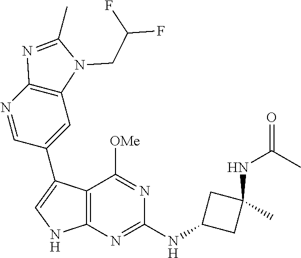 [SMILES: COc1nc(N[C@H]2C[C@](C)(NC(C)=O)C2)nc2[nH]cc(-c3cnc4nc(C)n(CC(F)F)c4c3)c12] | 2733 |
| 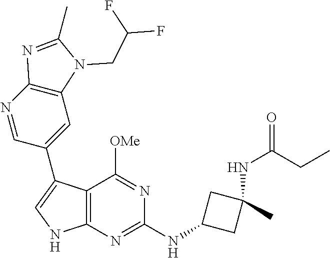 [SMILES: CCC(=O)N[C@]1(C)C[C@H](Nc2nc(OC)c3c(-c4cnc5nc(C)n(CC(F)F)c5c4)c[nH]c3n2)C1] | 2734 |
| 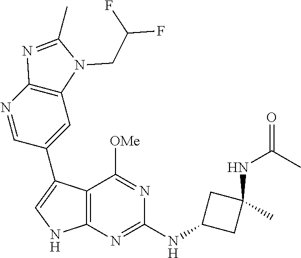 [SMILES: COc1nc(N[C@H]2C[C@](C)(NC(C)=O)C2)nc2[nH]cc(-c3cnc4nc(C)n(CC(F)F)c4c3)c12] | 2735 |
| 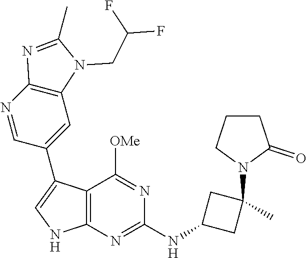 [SMILES: COc1nc(N[C@H]2C[C@](C)(N3CCCC3=O)C2)nc2[nH]cc(-c3cnc4nc(C)n(CC(F)F)c4c3)c12] | 2736 |
| 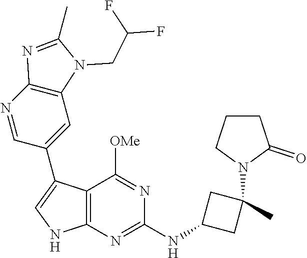 [SMILES: COc1nc(N[C@H]2C[C@@](C)(N3CCCC3=O)C2)nc2[nH]cc(-c3cnc4nc(C)n(CC(F)F)c4c3)c12] | 2737 |
| 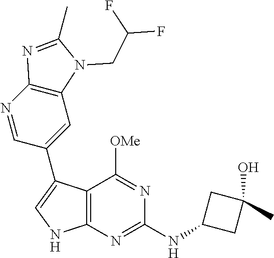 [SMILES: COc1nc(N[C@H]2C[C@@](C)(O)C2)nc2[nH]cc(-c3cnc4nc(C)n(CC(F)F)c4c3)c12] | 2738 |
| 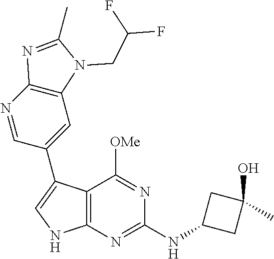 [SMILES: COc1nc(N[C@H]2C[C@](C)(O)C2)nc2[nH]cc(-c3cnc4nc(C)n(CC(F)F)c4c3)c12] | 2739 |
| 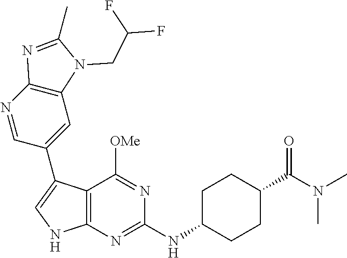 [SMILES: COc1nc(N[C@H]2CC[C@@H](C(=O)N(C)C)CC2)nc2[nH]cc(-c3cnc4nc(C)n(CC(F)F)c4c3)c12] | 2740 |
| 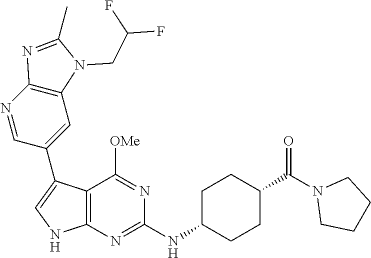 [SMILES: COc1nc(N[C@H]2CC[C@@H](C(=O)N3CCCC3)CC2)nc2[nH]cc(-c3cnc4nc(C)n(CC(F)F)c4c3)c12] | 2741 |
| 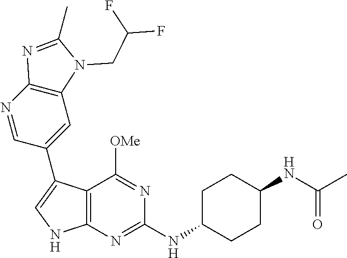 [SMILES: COc1nc(N[C@H]2CC[C@H](NC(C)=O)CC2)nc2[nH]cc(-c3cnc4nc(C)n(CC(F)F)c4c3)c12] | 2742 |
| 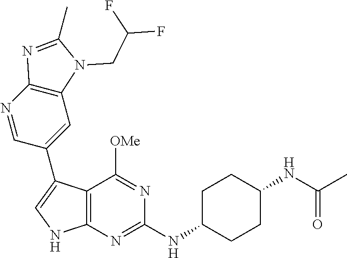 [SMILES: COc1nc(N[C@H]2CC[C@@H](NC(C)=O)CC2)nc2[nH]cc(-c3cnc4nc(C)n(CC(F)F)c4c3)c12] | 2743 |
| 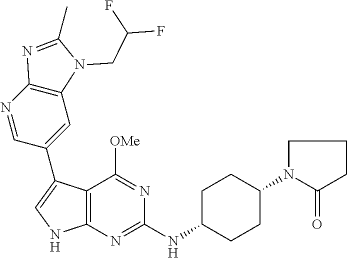 [SMILES: COc1nc(N[C@H]2CC[C@@H](N3CCCC3=O)CC2)nc2[nH]cc(-c3cnc4nc(C)n(CC(F)F)c4c3)c12] | 2744 |
| 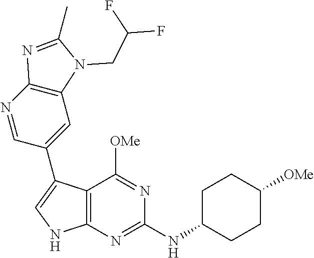 [SMILES: COc1nc(N[C@H]2CC[C@@H](OC)CC2)nc2[nH]cc(-c3cnc4nc(C)n(CC(F)F)c4c3)c12] | 2745 |
| 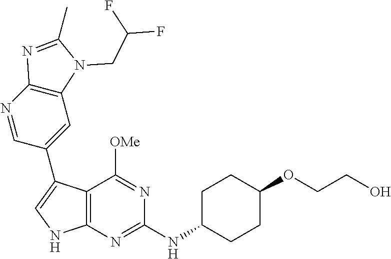 [SMILES: COc1nc(N[C@H]2CC[C@H](OCCO)CC2)nc2[nH]cc(-c3cnc4nc(C)n(CC(F)F)c4c3)c12] | 2746 |
| 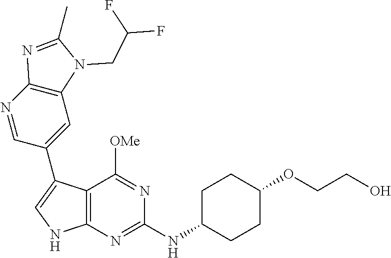 [SMILES: COc1nc(N[C@H]2CC[C@@H](OCCO)CC2)nc2[nH]cc(-c3cnc4nc(C)n(CC(F)F)c4c3)c12] | 2747 |
| 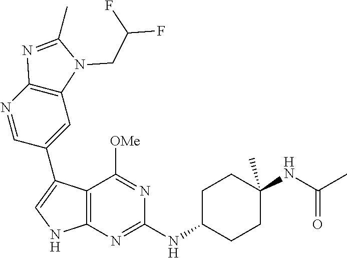 [SMILES: COc1nc(N[C@H]2CC[C@](C)(NC(C)=O)CC2)nc2[nH]cc(-c3cnc4nc(C)n(CC(F)F)c4c3)c12] | 2748 |
| 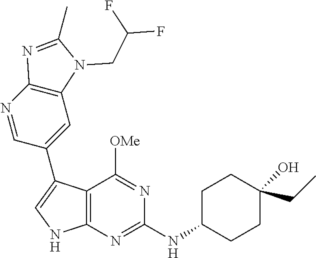 [SMILES: CC[C@]1(O)CC[C@@H](Nc2nc(OC)c3c(-c4cnc5nc(C)n(CC(F)F)c5c4)c[nH]c3n2)CC1] | 2749 |
| 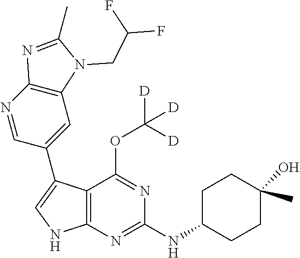 [SMILES: [2H]C([2H])([2H])Oc1nc(N[C@H]2CC[C@@](C)(O)CC2)nc2[nH]cc(-c3cnc4nc(C)n(CC(F)F)c4c3)c12] | 2750 |
| 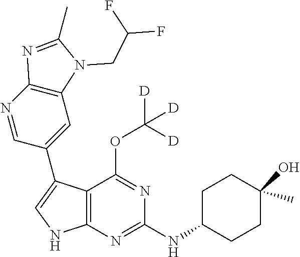 [SMILES: [2H]C([2H])([2H])Oc1nc(N[C@H]2CC[C@](C)(O)CC2)nc2[nH]cc(-c3cnc4nc(C)n(CC(F)F)c4c3)c12] | 2751 |
| 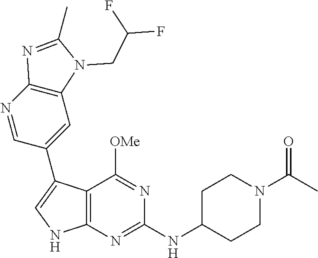 [SMILES: COc1nc(NC2CCN(C(C)=O)CC2)nc2[nH]cc(-c3cnc4nc(C)n(CC(F)F)c4c3)c12] | 2752 |
| 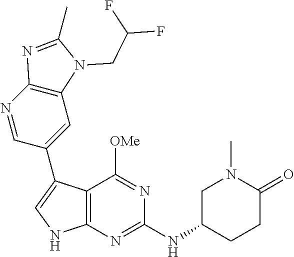 [SMILES: COc1nc(N[C@H]2CCC(=O)N(C)C2)nc2[nH]cc(-c3cnc4nc(C)n(CC(F)F)c4c3)c12] | 2753 |
| 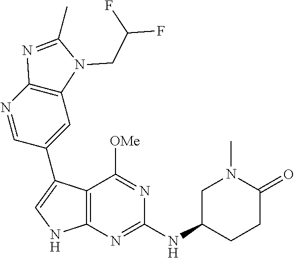 [SMILES: COc1nc(N[C@@H]2CCC(=O)N(C)C2)nc2[nH]cc(-c3cnc4nc(C)n(CC(F)F)c4c3)c12] | 2754 |
| 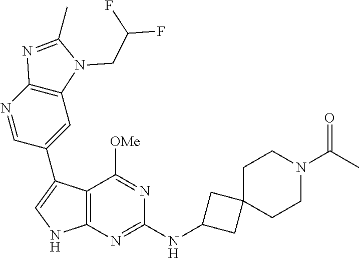 [SMILES: COc1nc(NC2CC3(CCN(C(C)=O)CC3)C2)nc2[nH]cc(-c3cnc4nc(C)n(CC(F)F)c4c3)c12] | 2755 |
| 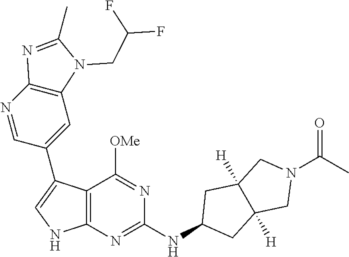 [SMILES: COc1nc(N[C@@H]2C[C@@H]3CN(C(C)=O)C[C@@H]3C2)nc2[nH]cc(-c3cnc4nc(C)n(CC(F)F)c4c3)c12] | 2756 |
| 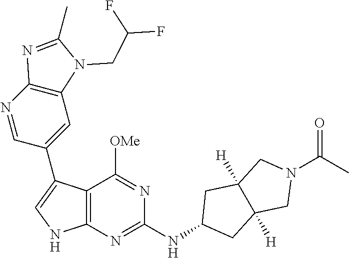 [SMILES: COc1nc(N[C@H]2C[C@@H]3CN(C(C)=O)C[C@@H]3C2)nc2[nH]cc(-c3cnc4nc(C)n(CC(F)F)c4c3)c12] | 2757 |
| 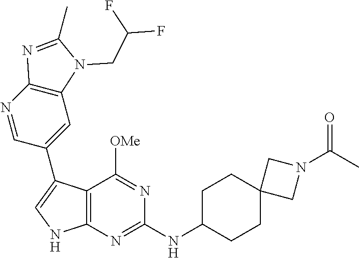 [SMILES: COc1nc(NC2CCC3(CC2)CN(C(C)=O)C3)nc2[nH]cc(-c3cnc4nc(C)n(CC(F)F)c4c3)c12] | 2758 |
| 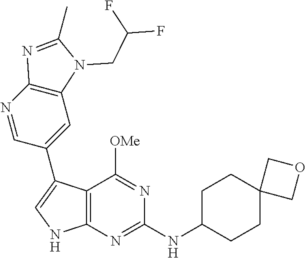 [SMILES: COc1nc(NC2CCC3(CC2)COC3)nc2[nH]cc(-c3cnc4nc(C)n(CC(F)F)c4c3)c12] | 2759 |
| 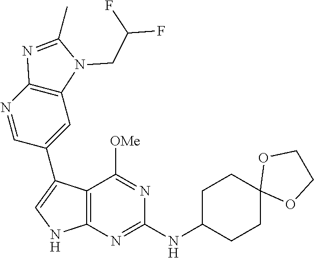 [SMILES: COc1nc(NC2CCC3(CC2)OCCO3)nc2[nH]cc(-c3cnc4nc(C)n(CC(F)F)c4c3)c12] | 2760 |
| 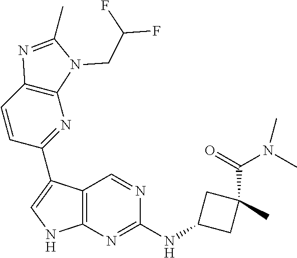 [SMILES: Cc1nc2ccc(-c3c[nH]c4nc(N[C@H]5C[C@@](C)(C(=O)N(C)C)C5)ncc34)nc2n1CC(F)F] | 2761 |
| 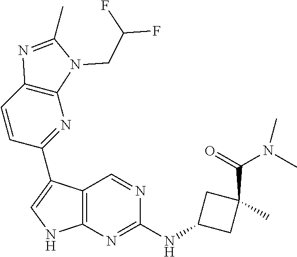 [SMILES: Cc1nc2ccc(-c3c[nH]c4nc(N[C@H]5C[C@](C)(C(=O)N(C)C)C5)ncc34)nc2n1CC(F)F] | 2762 |
| 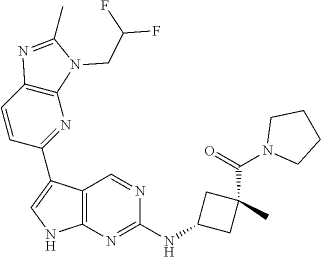 [SMILES: Cc1nc2ccc(-c3c[nH]c4nc(N[C@H]5C[C@@](C)(C(=O)N6CCCC6)C5)ncc34)nc2n1CC(F)F] | 2763 |
| 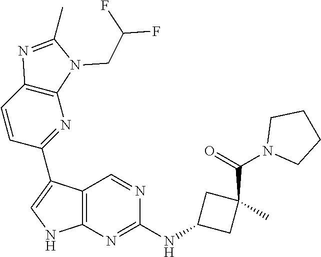 [SMILES: Cc1nc2ccc(-c3c[nH]c4nc(N[C@H]5C[C@](C)(C(=O)N6CCCC6)C5)ncc34)nc2n1CC(F)F] | 2764 |
| 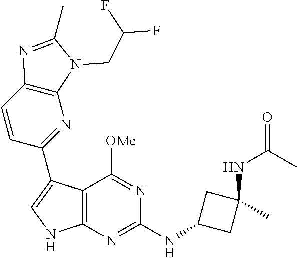 [SMILES: COc1nc(N[C@H]2C[C@](C)(NC(C)=O)C2)nc2[nH]cc(-c3ccc4nc(C)n(CC(F)F)c4n3)c12] | 2765 |
| 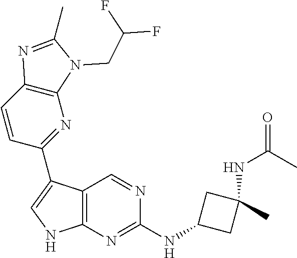 [SMILES: CC(=O)N[C@]1(C)C[C@H](Nc2ncc3c(-c4ccc5nc(C)n(CC(F)F)c5n4)c[nH]c3n2)C1] | 2766 |
| 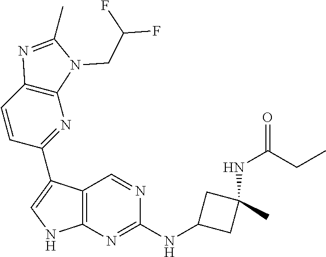 | 2767 |
| 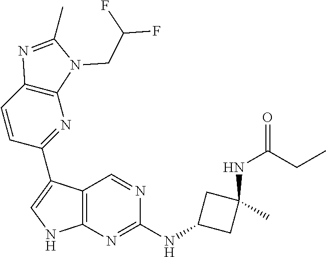 [SMILES: CCC(=O)N[C@]1(C)C[C@@H](Nc2ncc3c(-c4ccc5nc(C)n(CC(F)F)c5n4)c[nH]c3n2)C1] | 2768 |
| 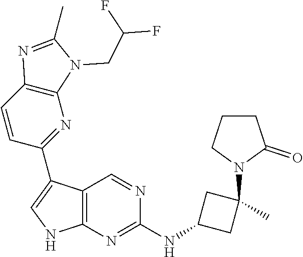 [SMILES: Cc1nc2ccc(-c3c[nH]c4nc(N[C@H]5C[C@](C)(N6CCCC6=O)C5)ncc34)nc2n1CC(F)F] | 2769 |
| 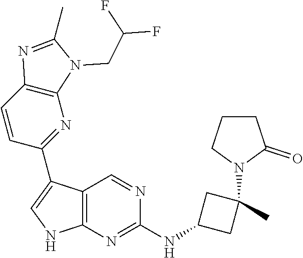 [SMILES: Cc1nc2ccc(-c3c[nH]c4nc(N[C@H]5C[C@@](C)(N6CCCC6=O)C5)ncc34)nc2n1CC(F)F] | 2770 |
| 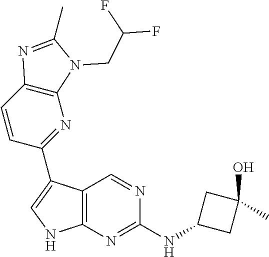 [SMILES: Cc1nc2ccc(-c3c[nH]c4nc(N[C@H]5C[C@](C)(O)C5)ncc34)nc2n1CC(F)F] | 2771 |
| 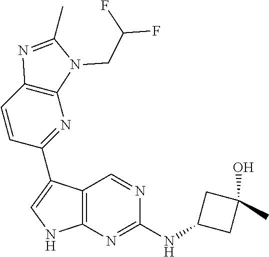 [SMILES: Cc1nc2ccc(-c3c[nH]c4nc(N[C@H]5C[C@@](C)(O)C5)ncc34)nc2n1CC(F)F] | 2772 |
| 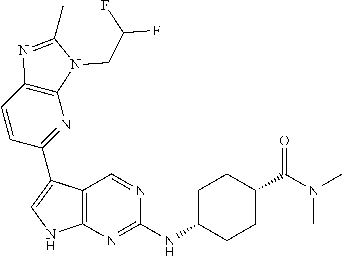 [SMILES: Cc1nc2ccc(-c3c[nH]c4nc(N[C@H]5CC[C@@H](C(=O)N(C)C)CC5)ncc34)nc2n1CC(F)F] | 2773 |
| 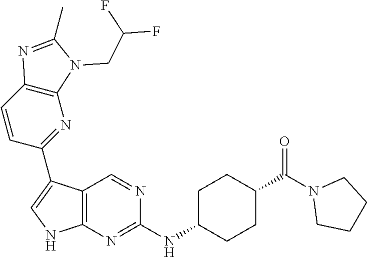 [SMILES: Cc1nc2ccc(-c3c[nH]c4nc(N[C@H]5CC[C@@H](C(=O)N6CCCC6)CC5)ncc34)nc2n1CC(F)F] | 2774 |
| 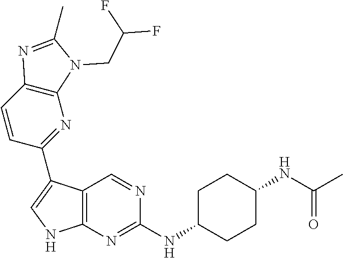 [SMILES: CC(=O)N[C@H]1CC[C@@H](Nc2ncc3c(-c4ccc5nc(C)n(CC(F)F)c5n4)c[nH]c3n2)CC1] | 2775 |
| 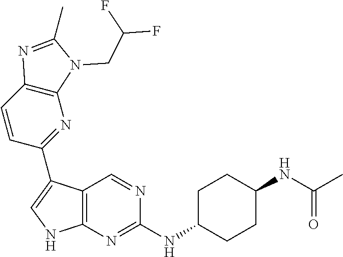 [SMILES: CC(=O)N[C@H]1CC[C@H](Nc2ncc3c(-c4ccc5nc(C)n(CC(F)F)c5n4)c[nH]c3n2)CC1] | 2776 |
| 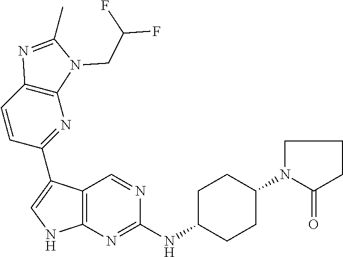 [SMILES: Cc1nc2ccc(-c3c[nH]c4nc(N[C@H]5CC[C@@H](N6CCCC6=O)CC5)ncc34)nc2n1CC(F)F] | 2777 |
| 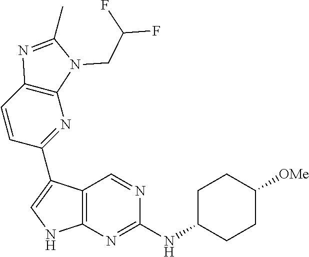 [SMILES: CO[C@H]1CC[C@@H](Nc2ncc3c(-c4ccc5nc(C)n(CC(F)F)c5n4)c[nH]c3n2)CC1] | 2778 |
| 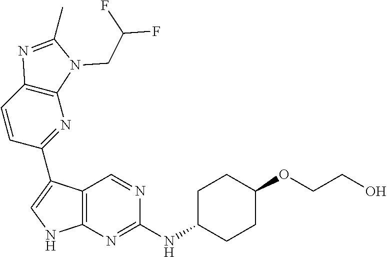 [SMILES: Cc1nc2ccc(-c3c[nH]c4nc(N[C@H]5CC[C@H](OCCO)CC5)ncc34)nc2n1CC(F)F] | 2779 |
| 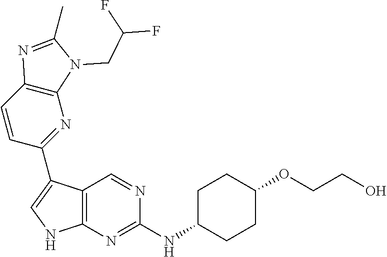 [SMILES: Cc1nc2ccc(-c3c[nH]c4nc(N[C@H]5CC[C@@H](OCCO)CC5)ncc34)nc2n1CC(F)F] | 2780 |
| 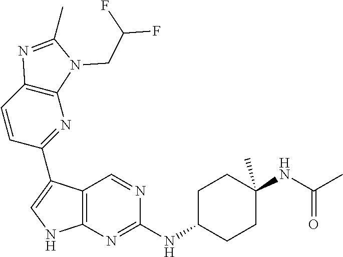 [SMILES: CC(=O)N[C@]1(C)CC[C@@H](Nc2ncc3c(-c4ccc5nc(C)n(CC(F)F)c5n4)c[nH]c3n2)CC1] | 2781 |
| 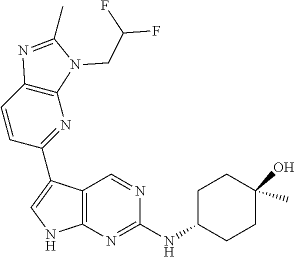 [SMILES: Cc1nc2ccc(-c3c[nH]c4nc(N[C@H]5CC[C@](C)(O)CC5)ncc34)nc2n1CC(F)F] | 2782 |
| 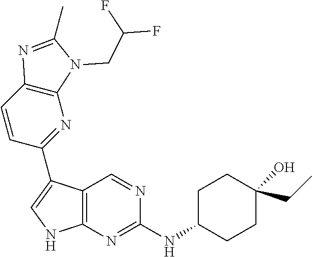 [SMILES: CC[C@]1(O)CC[C@@H](Nc2ncc3c(-c4ccc5nc(C)n(CC(F)F)c5n4)c[nH]c3n2)CC1] | 2783 |
| 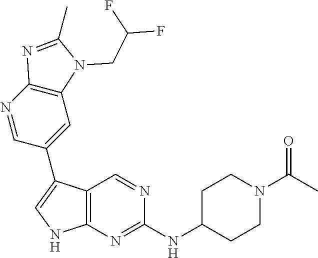 [SMILES: CC(=O)N1CCC(Nc2ncc3c(-c4cnc5nc(C)n(CC(F)F)c5c4)c[nH]c3n2)CC1] | 2784 |
| 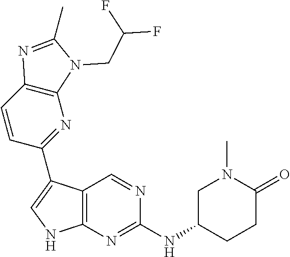 [SMILES: Cc1nc2ccc(-c3c[nH]c4nc(N[C@H]5CCC(=O)N(C)C5)ncc34)nc2n1CC(F)F] | 2785 |
| 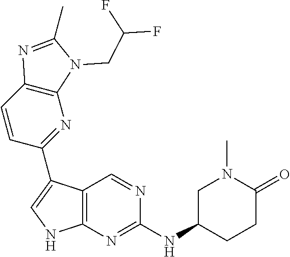 [SMILES: Cc1nc2ccc(-c3c[nH]c4nc(N[C@@H]5CCC(=O)N(C)C5)ncc34)nc2n1CC(F)F] | 2786 |
| 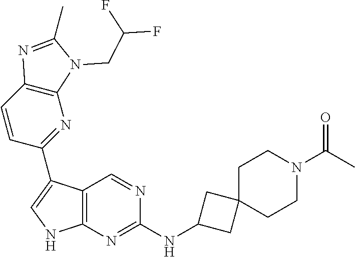 [SMILES: CC(=O)N1CCC2(CC1)CC(Nc1ncc3c(-c4ccc5nc(C)n(CC(F)F)c5n4)c[nH]c3n1)C2] | 2787 |
| 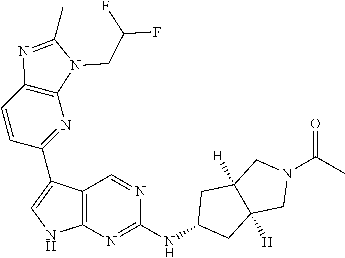 [SMILES: CC(=O)N1C[C@H]2C[C@H](Nc3ncc4c(-c5ccc6nc(C)n(CC(F)F)c6n5)c[nH]c4n3)C[C@H]2C1] | 2788 |
| 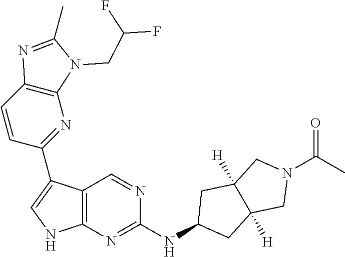 [SMILES: CC(=O)N1C[C@H]2C[C@@H](Nc3ncc4c(-c5ccc6nc(C)n(CC(F)F)c6n5)c[nH]c4n3)C[C@H]2C1] | 2789 |
| 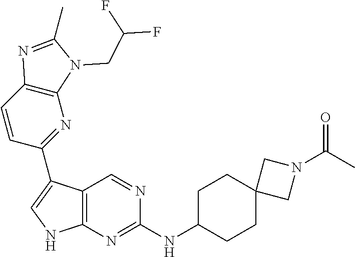 [SMILES: CC(=O)N1CC2(CCC(Nc3ncc4c(-c5ccc6nc(C)n(CC(F)F)c6n5)c[nH]c4n3)CC2)C1] | 2790 |
| 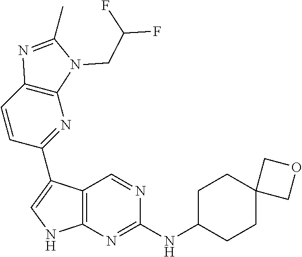 [SMILES: Cc1nc2ccc(-c3c[nH]c4nc(NC5CCC6(CC5)COC6)ncc34)nc2n1CC(F)F] | 2791 |
| 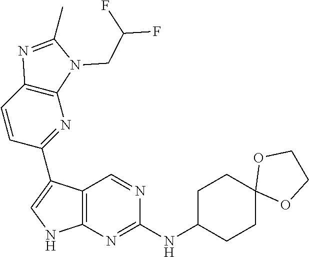 [SMILES: Cc1nc2ccc(-c3c[nH]c4nc(NC5CCC6(CC5)OCCO6)ncc34)nc2n1CC(F)F] | 2792 |
| 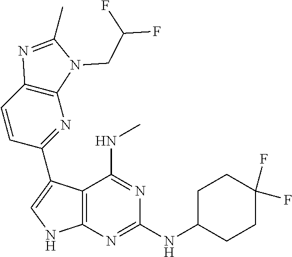 [SMILES: CNc1nc(NC2CCC(F)(F)CC2)nc2[nH]cc(-c3ccc4nc(C)n(CC(F)F)c4n3)c12] | 2793 |
| 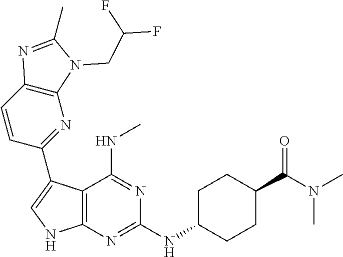 [SMILES: CNc1nc(N[C@H]2CC[C@H](C(=O)N(C)C)CC2)nc2[nH]cc(-c3ccc4nc(C)n(CC(F)F)c4n3)c12] | 2794 |
| 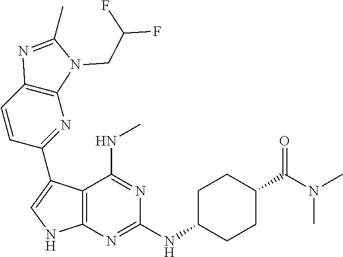 [SMILES: CNc1nc(N[C@H]2CC[C@@H](C(=O)N(C)C)CC2)nc2[nH]cc(-c3ccc4nc(C)n(CC(F)F)c4n3)c12] | 2795 |
| 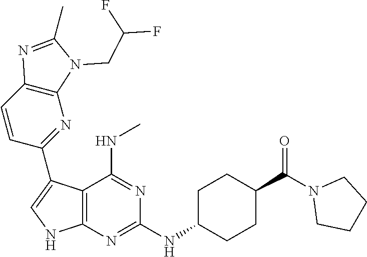 [SMILES: CNc1nc(N[C@H]2CC[C@H](C(=O)N3CCCC3)CC2)nc2[nH]cc(-c3ccc4nc(C)n(CC(F)F)c4n3)c12] | 2796 |
| 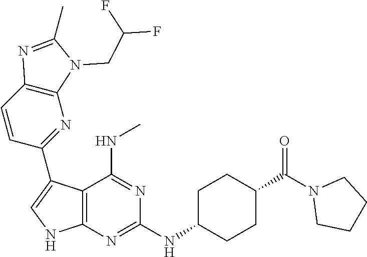 [SMILES: CNc1nc(N[C@H]2CC[C@@H](C(=O)N3CCCC3)CC2)nc2[nH]cc(-c3ccc4nc(C)n(CC(F)F)c4n3)c12] | 2797 |
| 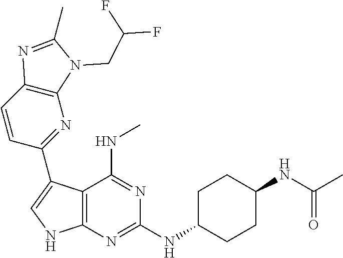 [SMILES: CNc1nc(N[C@H]2CC[C@H](NC(C)=O)CC2)nc2[nH]cc(-c3ccc4nc(C)n(CC(F)F)c4n3)c12] | 2798 |
| 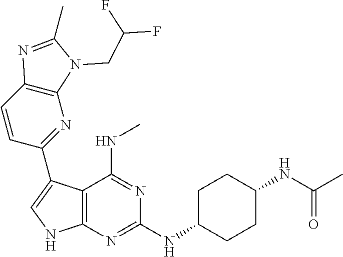 [SMILES: CNc1nc(N[C@H]2CC[C@@H](NC(C)=O)CC2)nc2[nH]cc(-c3ccc4nc(C)n(CC(F)F)c4n3)c12] | 2799 |
| 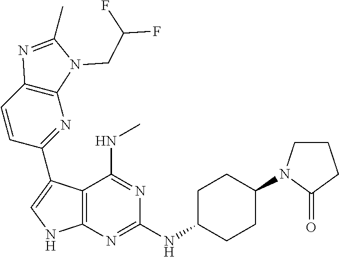 [SMILES: CNc1nc(N[C@H]2CC[C@H](N3CCCC3=O)CC2)nc2[nH]cc(-c3ccc4nc(C)n(CC(F)F)c4n3)c12] | 2800 |
| 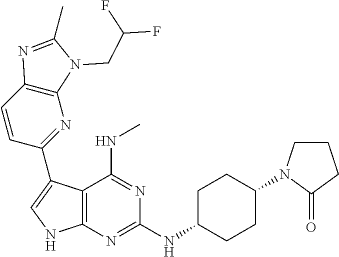 [SMILES: CNc1nc(N[C@H]2CC[C@@H](N3CCCC3=O)CC2)nc2[nH]cc(-c3ccc4nc(C)n(CC(F)F)c4n3)c12] | 2801 |
| 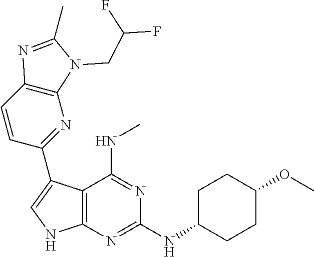 [SMILES: CNc1nc(N[C@H]2CC[C@@H](OC)CC2)nc2[nH]cc(-c3ccc4nc(C)n(CC(F)F)c4n3)c12] | 2802 |
| 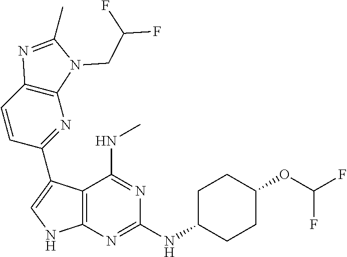 [SMILES: CNc1nc(N[C@H]2CC[C@@H](OC(F)F)CC2)nc2[nH]cc(-c3ccc4nc(C)n(CC(F)F)c4n3)c12] | 2803 |
| 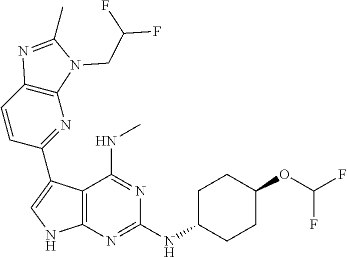 [SMILES: CNc1nc(N[C@H]2CC[C@H](OC(F)F)CC2)nc2[nH]cc(-c3ccc4nc(C)n(CC(F)F)c4n3)c12] | 2804 |
| 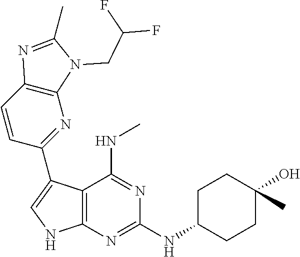 [SMILES: CNc1nc(N[C@H]2CC[C@@](C)(O)CC2)nc2[nH]cc(-c3ccc4nc(C)n(CC(F)F)c4n3)c12] | 2805 |
| 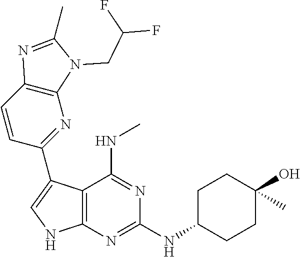 [SMILES: CNc1nc(N[C@H]2CC[C@](C)(O)CC2)nc2[nH]cc(-c3ccc4nc(C)n(CC(F)F)c4n3)c12] | 2806 |
| 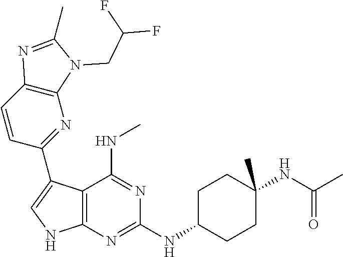 [SMILES: CNc1nc(N[C@H]2CC[C@@](C)(NC(C)=O)CC2)nc2[nH]cc(-c3ccc4nc(C)n(CC(F)F)c4n3)c12] | 2807 |
| 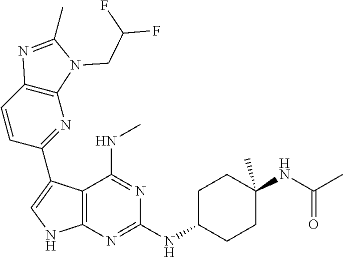 [SMILES: CNc1nc(N[C@H]2CC[C@](C)(NC(C)=O)CC2)nc2[nH]cc(-c3ccc4nc(C)n(CC(F)F)c4n3)c12] | 2808 |
| 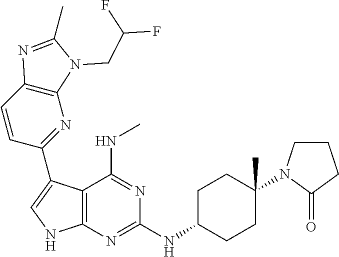 [SMILES: CNc1nc(N[C@H]2CC[C@@](C)(N3CCCC3=O)CC2)nc2[nH]cc(-c3ccc4nc(C)n(CC(F)F)c4n3)c12] | 2809 |
| 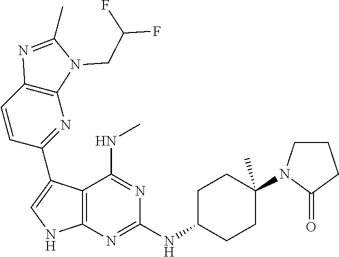 [SMILES: CNc1nc(N[C@H]2CC[C@](C)(N3CCCC3=O)CC2)nc2[nH]cc(-c3ccc4nc(C)n(CC(F)F)c4n3)c12] | 2810 |
| 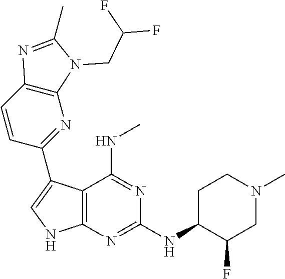 [SMILES: CNc1nc(N[C@H]2CCN(C)C[C@H]2F)nc2[nH]cc(-c3ccc4nc(C)n(CC(F)F)c4n3)c12] | 2811 |
| 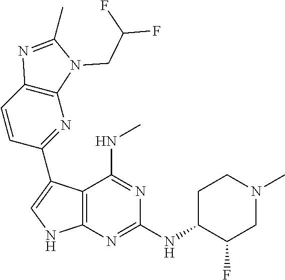 [SMILES: CNc1nc(N[C@@H]2CCN(C)C[C@@H]2F)nc2[nH]cc(-c3ccc4nc(C)n(CC(F)F)c4n3)c12] | 2812 |
| 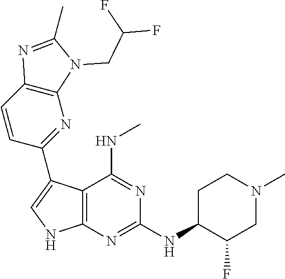 [SMILES: CNc1nc(N[C@H]2CCN(C)C[C@@H]2F)nc2[nH]cc(-c3ccc4nc(C)n(CC(F)F)c4n3)c12] | 2813 |
| 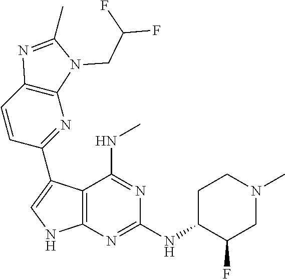 [SMILES: CNc1nc(N[C@@H]2CCN(C)C[C@H]2F)nc2[nH]cc(-c3ccc4nc(C)n(CC(F)F)c4n3)c12] | 2814 |
| 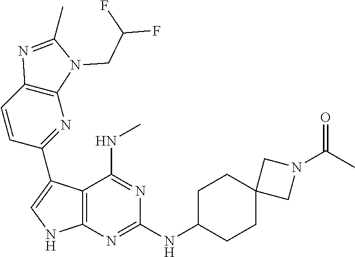 [SMILES: CNc1nc(NC2CCC3(CC2)CN(C(C)=O)C3)nc2[nH]cc(-c3ccc4nc(C)n(CC(F)F)c4n3)c12] | 2815 |
| 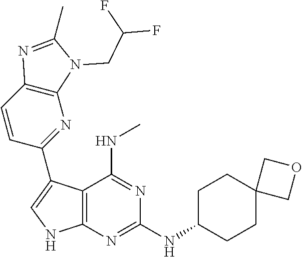 | 2816 |
| 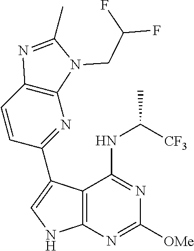 [SMILES: COc1nc(N[C@H](C)C(F)(F)F)c2c(-c3ccc4nc(C)n(CC(F)F)c4n3)c[nH]c2n1] | 2817 |
| 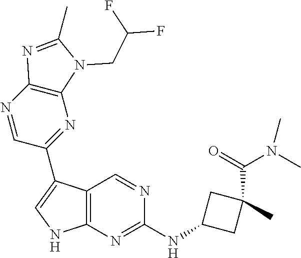 [SMILES: Cc1nc2ncc(-c3c[nH]c4nc(N[C@H]5C[C@@](C)(C(=O)N(C)C)C5)ncc34)nc2n1CC(F)F] | 2818 |
| 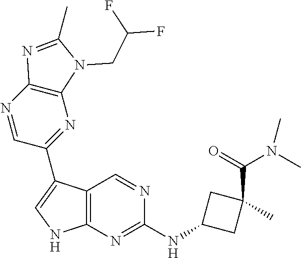 [SMILES: Cc1nc2ncc(-c3c[nH]c4nc(N[C@H]5C[C@](C)(C(=O)N(C)C)C5)ncc34)nc2n1CC(F)F] | 2819 |
| 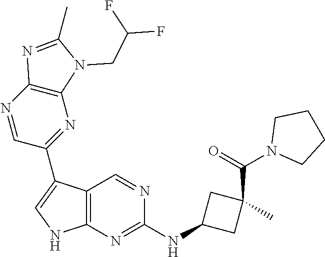 [SMILES: Cc1nc2ncc(-c3c[nH]c4nc(N[C@H]5C[C@@](C)(C(=O)N6CCCC6)C5)ncc34)nc2n1CC(F)F] | 2820 |
| 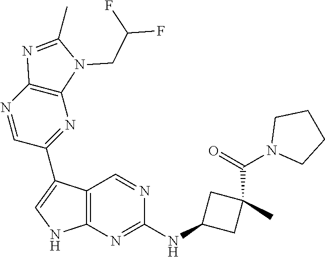 [SMILES: Cc1nc2ncc(-c3c[nH]c4nc(N[C@H]5C[C@](C)(C(=O)N6CCCC6)C5)ncc34)nc2n1CC(F)F] | 2821 |
| 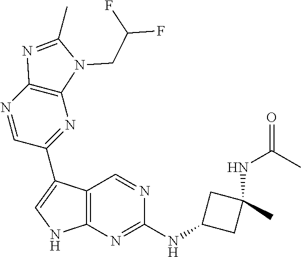 [SMILES: CC(=O)N[C@]1(C)C[C@H](Nc2ncc3c(-c4cnc5nc(C)n(CC(F)F)c5n4)c[nH]c3n2)C1] | 2822 |
| 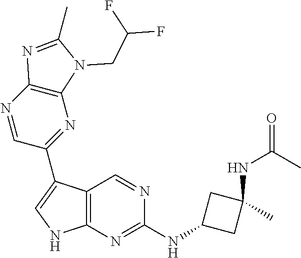 [SMILES: CC(=O)N[C@]1(C)C[C@@H](Nc2ncc3c(-c4cnc5nc(C)n(CC(F)F)c5n4)c[nH]c3n2)C1] | 2823 |
| 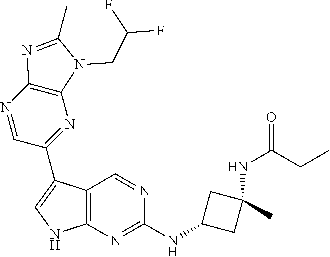 [SMILES: CCC(=O)N[C@]1(C)C[C@H](Nc2ncc3c(-c4cnc5nc(C)n(CC(F)F)c5n4)c[nH]c3n2)C1] | 2824 |
| 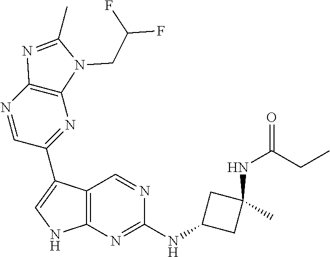 [SMILES: CCC(=O)N[C@]1(C)C[C@@H](Nc2ncc3c(-c4cnc5nc(C)n(CC(F)F)c5n4)c[nH]c3n2)C1] | 2825 |
| 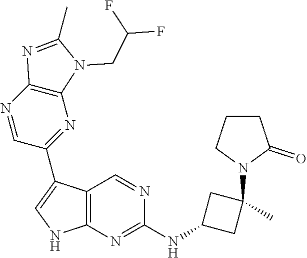 [SMILES: Cc1nc2ncc(-c3c[nH]c4nc(N[C@H]5C[C@](C)(N6CCCC6=O)C5)ncc34)nc2n1CC(F)F] | 2826 |
| 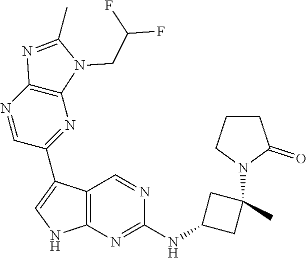 [SMILES: Cc1nc2ncc(-c3c[nH]c4nc(N[C@H]5C[C@@](C)(N6CCCC6=O)C5)ncc34)nc2n1CC(F)F] | 2827 |
| 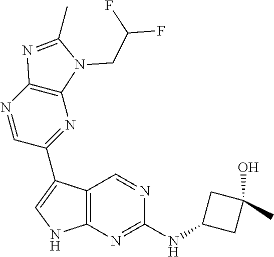 [SMILES: Cc1nc2ncc(-c3c[nH]c4nc(N[C@H]5C[C@@](C)(O)C5)ncc34)nc2n1CC(F)F] | 2828 |
| 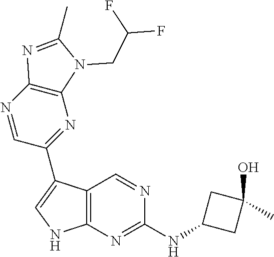 [SMILES: Cc1nc2ncc(-c3c[nH]c4nc(N[C@H]5C[C@](C)(O)C5)ncc34)nc2n1CC(F)F] | 2829 |
| 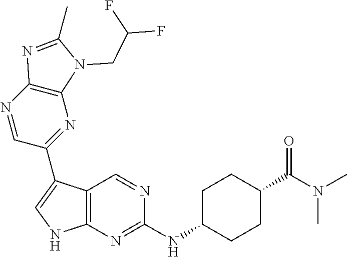 [SMILES: Cc1nc2ncc(-c3c[nH]c4nc(N[C@H]5CC[C@@H](C(=O)N(C)C)CC5)ncc34)nc2n1CC(F)F] | 2830 |
| 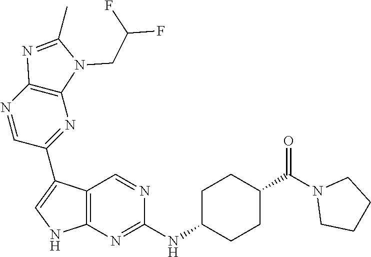 [SMILES: Cc1nc2ncc(-c3c[nH]c4nc(N[C@H]5CC[C@@H](C(=O)N6CCCC6)CC5)ncc34)nc2n1CC(F)F] | 2831 |
| 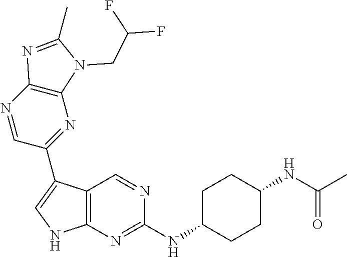 [SMILES: CC(=O)N[C@H]1CC[C@@H](Nc2ncc3c(-c4cnc5nc(C)n(CC(F)F)c5n4)c[nH]c3n2)CC1] | 2832 |
| 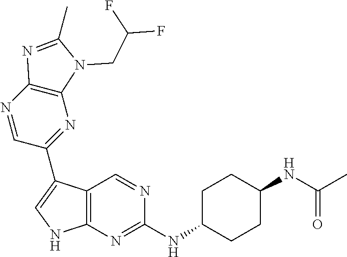 [SMILES: CC(=O)N[C@H]1CC[C@H](Nc2ncc3c(-c4cnc5nc(C)n(CC(F)F)c5n4)c[nH]c3n2)CC1] | 2833 |
| 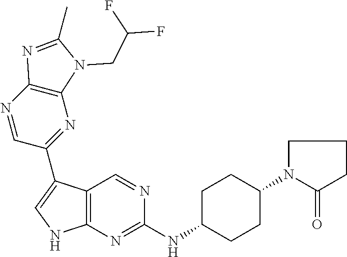 [SMILES: Cc1nc2ncc(-c3c[nH]c4nc(N[C@H]5CC[C@@H](N6CCCC6=O)CC5)ncc34)nc2n1CC(F)F] | 2834 |
| 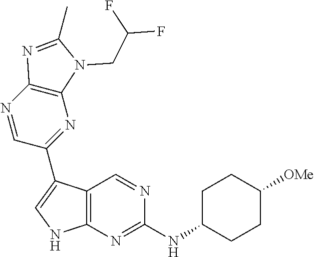 [SMILES: CO[C@H]1CC[C@@H](Nc2ncc3c(-c4cnc5nc(C)n(CC(F)F)c5n4)c[nH]c3n2)CC1] | 2835 |
| 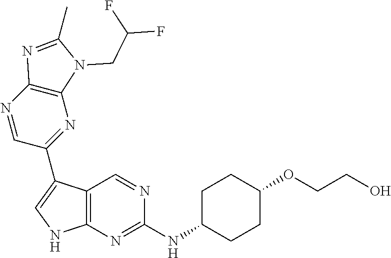 [SMILES: Cc1nc2ncc(-c3c[nH]c4nc(N[C@H]5CC[C@@H](OCCO)CC5)ncc34)nc2n1CC(F)F] | 2836 |
| 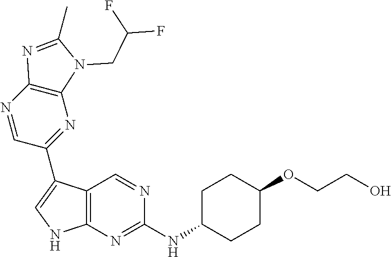 [SMILES: Cc1nc2ncc(-c3c[nH]c4nc(N[C@H]5CC[C@H](OCCO)CC5)ncc34)nc2n1CC(F)F] | 2837 |
| 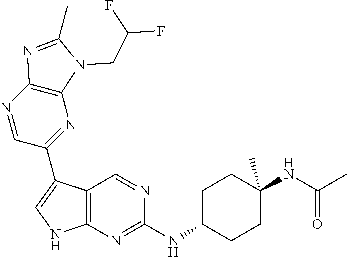 [SMILES: CC(=O)N[C@]1(C)CC[C@@H](Nc2ncc3c(-c4cnc5nc(C)n(CC(F)F)c5n4)c[nH]c3n2)CC1] | 2838 |
| 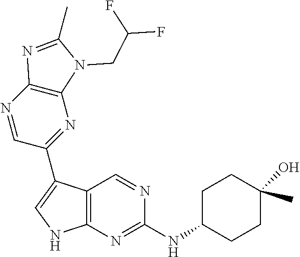 [SMILES: Cc1nc2ncc(-c3c[nH]c4nc(N[C@H]5CC[C@@](C)(O)CC5)ncc34)nc2n1CC(F)F] | 2839 |
| 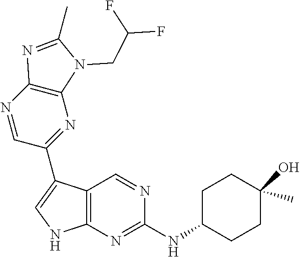 [SMILES: Cc1nc2ncc(-c3c[nH]c4nc(N[C@H]5CC[C@](C)(O)CC5)ncc34)nc2n1CC(F)F] | 2840 |
| 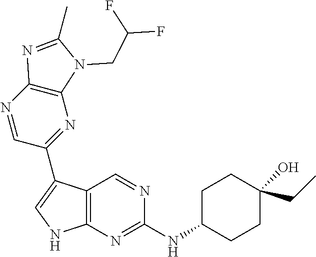 [SMILES: CC[C@]1(O)CC[C@@H](Nc2ncc3c(-c4cnc5nc(C)n(CC(F)F)c5n4)c[nH]c3n2)CC1] | 2841 |
| 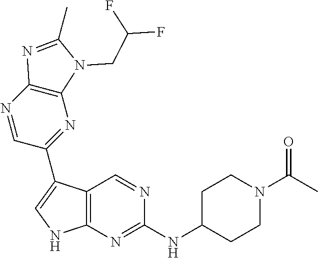 [SMILES: CC(=O)N1CCC(Nc2ncc3c(-c4cnc5nc(C)n(CC(F)F)c5n4)c[nH]c3n2)CC1] | 2842 |
| 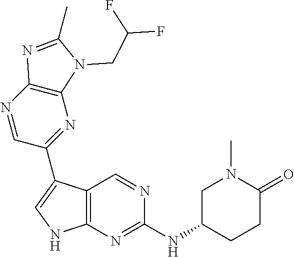 [SMILES: Cc1nc2ncc(-c3c[nH]c4nc(N[C@H]5CCC(=O)N(C)C5)ncc34)nc2n1CC(F)F] | 2843 |
| 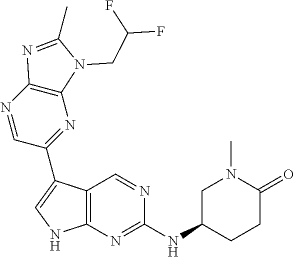 [SMILES: Cc1nc2ncc(-c3c[nH]c4nc(N[C@@H]5CCC(=O)N(C)C5)ncc34)nc2n1CC(F)F] | 2844 |
| 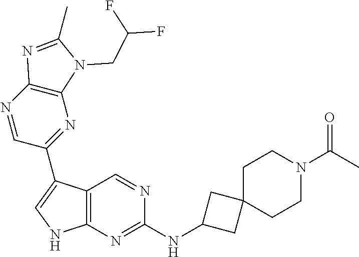 [SMILES: CC(=O)N1CCC2(CC1)CC(Nc1ncc3c(-c4cnc5nc(C)n(CC(F)F)c5n4)c[nH]c3n1)C2] | 2845 |
| 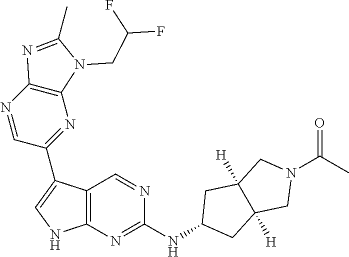 [SMILES: CC(=O)N1C[C@H]2C[C@H](Nc3ncc4c(-c5cnc6nc(C)n(CC(F)F)c6n5)c[nH]c4n3)C[C@H]2C1] | 2846 |
| 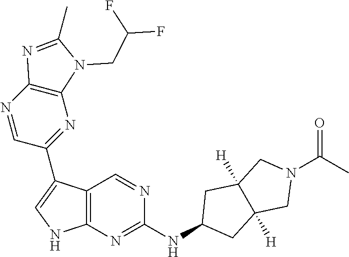 [SMILES: CC(=O)N1C[C@H]2C[C@@H](Nc3ncc4c(-c5cnc6nc(C)n(CC(F)F)c6n5)c[nH]c4n3)C[C@H]2C1] | 2847 |
| 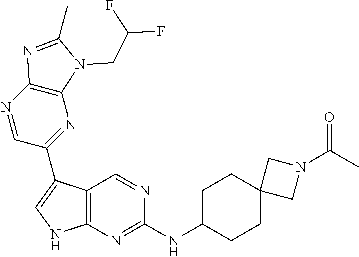 [SMILES: CC(=O)N1CC2(CCC(Nc3ncc4c(-c5cnc6nc(C)n(CC(F)F)c6n5)c[nH]c4n3)CC2)C1] | 2848 |
| 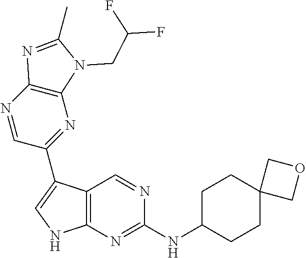 [SMILES: Cc1nc2ncc(-c3c[nH]c4nc(NC5CCC6(CC5)COC6)ncc34)nc2n1CC(F)F] | 2849 |
| 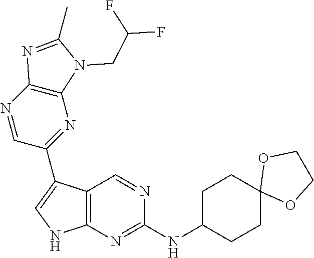 [SMILES: Cc1nc2ncc(-c3c[nH]c4nc(NC5CCC6(CC5)OCCO6)ncc34)nc2n1CC(F)F] | 2850 |
| 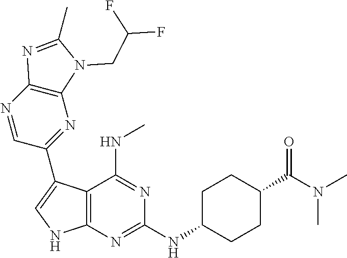 [SMILES: CNc1nc(N[C@H]2CC[C@@H](C(=O)N(C)C)CC2)nc2[nH]cc(-c3cnc4nc(C)n(CC(F)F)c4n3)c12] | 2851 |
| 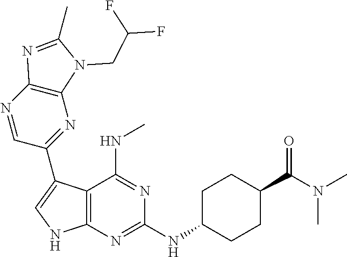 [SMILES: CNc1nc(N[C@H]2CC[C@H](C(=O)N(C)C)CC2)nc2[nH]cc(-c3cnc4nc(C)n(CC(F)F)c4n3)c12] | 2852 |
| 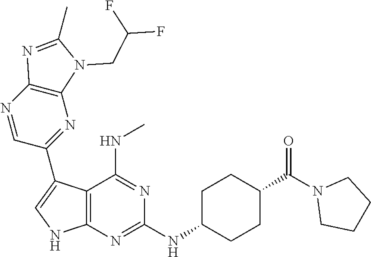 [SMILES: CNc1nc(N[C@H]2CC[C@@H](C(=O)N3CCCC3)CC2)nc2[nH]cc(-c3cnc4nc(C)n(CC(F)F)c4n3)c12] | 2853 |
| 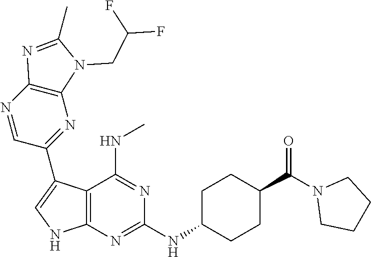 [SMILES: CNc1nc(N[C@H]2CC[C@H](C(=O)N3CCCC3)CC2)nc2[nH]cc(-c3cnc4nc(C)n(CC(F)F)c4n3)c12] | 2854 |
| 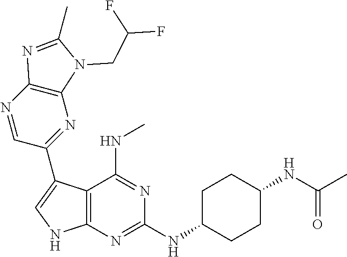 [SMILES: CNc1nc(N[C@H]2CC[C@@H](NC(C)=O)CC2)nc2[nH]cc(-c3cnc4nc(C)n(CC(F)F)c4n3)c12] | 2855 |
| 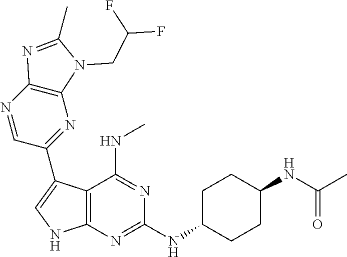 [SMILES: CNc1nc(N[C@H]2CC[C@H](NC(C)=O)CC2)nc2[nH]cc(-c3cnc4nc(C)n(CC(F)F)c4n3)c12] | 2856 |
| 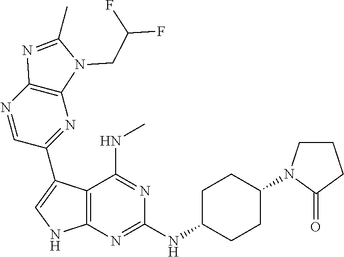 [SMILES: CNc1nc(N[C@H]2CC[C@@H](N3CCCC3=O)CC2)nc2[nH]cc(-c3cnc4nc(C)n(CC(F)F)c4n3)c12] | 2857 |
| 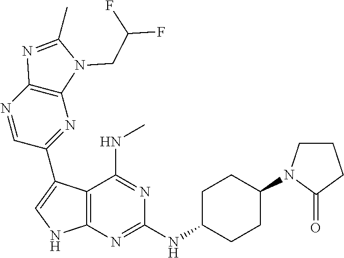 [SMILES: CNc1nc(N[C@H]2CC[C@H](N3CCCC3=O)CC2)nc2[nH]cc(-c3cnc4nc(C)n(CC(F)F)c4n3)c12] | 2858 |
| 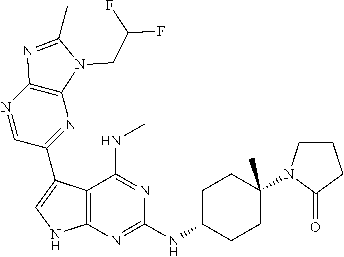 [SMILES: CNc1nc(N[C@H]2CC[C@@](C)(N3CCCC3=O)CC2)nc2[nH]cc(-c3cnc4nc(C)n(CC(F)F)c4n3)c12] | 2859 |
| 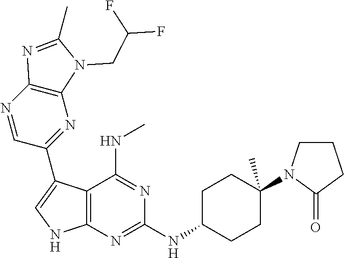 [SMILES: CNc1nc(N[C@H]2CC[C@](C)(N3CCCC3=O)CC2)nc2[nH]cc(-c3cnc4nc(C)n(CC(F)F)c4n3)c12] | 2860 |
| 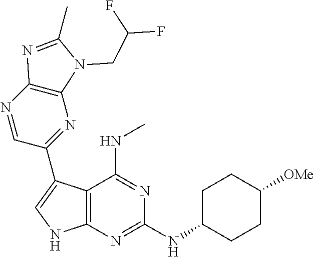 [SMILES: CNc1nc(N[C@H]2CC[C@@H](OC)CC2)nc2[nH]cc(-c3cnc4nc(C)n(CC(F)F)c4n3)c12] | 2861 |
| 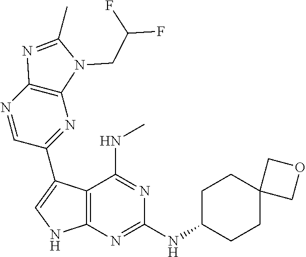 | 2862 |
| 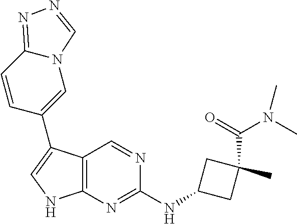 [SMILES: CN(C)C(=O)[C@]1(C)C[C@H](Nc2ncc3c(-c4ccc5nncn5c4)c[nH]c3n2)C1] | 2863 |
| 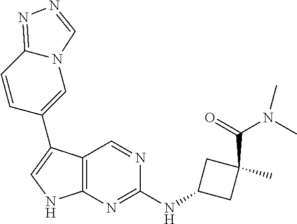 [SMILES: CN(C)C(=O)[C@]1(C)C[C@@H](Nc2ncc3c(-c4ccc5nncn5c4)c[nH]c3n2)C1] | 2864 |
| 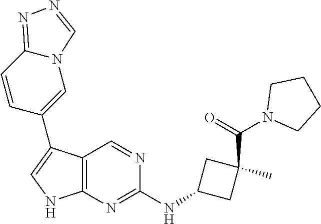 [SMILES: C[C@]1(C(=O)N2CCCC2)C[C@H](Nc2ncc3c(-c4ccc5nncn5c4)c[nH]c3n2)C1] | 2865 |
| 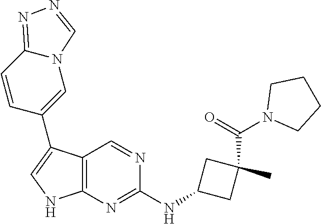 [SMILES: C[C@]1(C(=O)N2CCCC2)C[C@@H](Nc2ncc3c(-c4ccc5nncn5c4)c[nH]c3n2)C1] | 2866 |
| 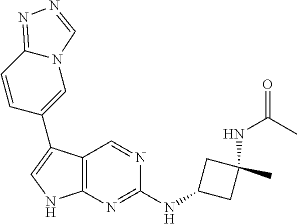 [SMILES: CC(=O)N[C@]1(C)C[C@H](Nc2ncc3c(-c4ccc5nncn5c4)c[nH]c3n2)C1] | 2867 |
| 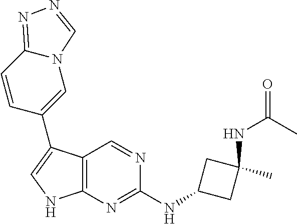 [SMILES: CC(=O)N[C@]1(C)C[C@@H](Nc2ncc3c(-c4ccc5nncn5c4)c[nH]c3n2)C1] | 2868 |
| 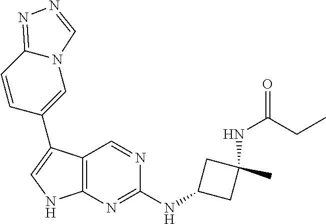 [SMILES: CCC(=O)N[C@]1(C)C[C@H](Nc2ncc3c(-c4ccc5nncn5c4)c[nH]c3n2)C1] | 2869 |
| 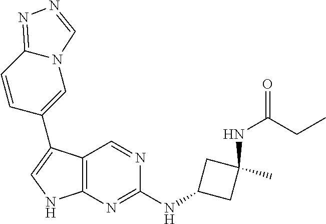 [SMILES: CCC(=O)N[C@]1(C)C[C@@H](Nc2ncc3c(-c4ccc5nncn5c4)c[nH]c3n2)C1] | 2870 |
| 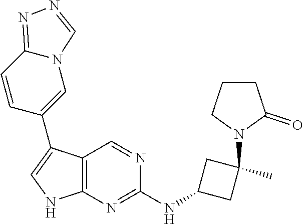 [SMILES: C[C@]1(N2CCCC2=O)C[C@H](Nc2ncc3c(-c4ccc5nncn5c4)c[nH]c3n2)C1] | 2871 |
| 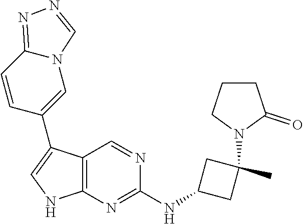 [SMILES: C[C@]1(N2CCCC2=O)C[C@@H](Nc2ncc3c(-c4ccc5nncn5c4)c[nH]c3n2)C1] | 2872 |
| 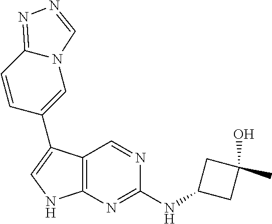 [SMILES: C[C@]1(O)C[C@@H](Nc2ncc3c(-c4ccc5nncn5c4)c[nH]c3n2)C1] | 2873 |
| 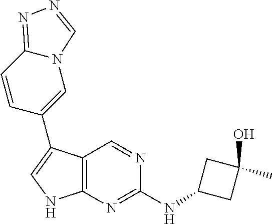 [SMILES: C[C@]1(O)C[C@H](Nc2ncc3c(-c4ccc5nncn5c4)c[nH]c3n2)C1] | 2874 |
| 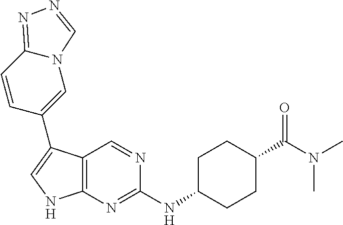 [SMILES: CN(C)C(=O)[C@H]1CC[C@@H](Nc2ncc3c(-c4ccc5nncn5c4)c[nH]c3n2)CC1] | 2875 |
| 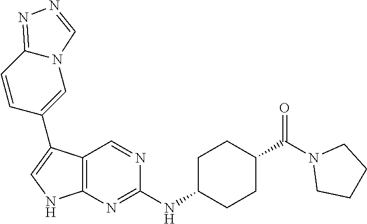 [SMILES: O=C([C@H]1CC[C@@H](Nc2ncc3c(-c4ccc5nncn5c4)c[nH]c3n2)CC1)N1CCCC1] | 2876 |
| 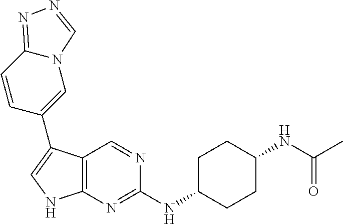 [SMILES: CC(=O)N[C@H]1CC[C@@H](Nc2ncc3c(-c4ccc5nncn5c4)c[nH]c3n2)CC1] | 2877 |
| 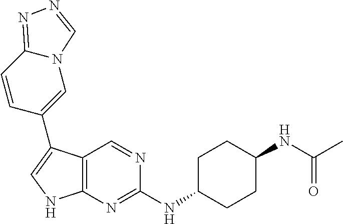 [SMILES: CC(=O)N[C@H]1CC[C@H](Nc2ncc3c(-c4ccc5nncn5c4)c[nH]c3n2)CC1] | 2878 |
| 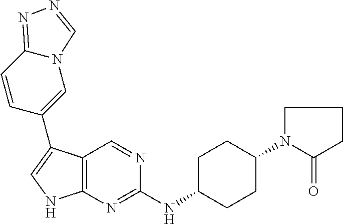 [SMILES: O=C1CCCN1[C@H]1CC[C@@H](Nc2ncc3c(-c4ccc5nncn5c4)c[nH]c3n2)CC1] | 2879 |
| 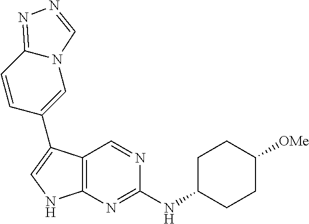 [SMILES: CO[C@H]1CC[C@@H](Nc2ncc3c(-c4ccc5nncn5c4)c[nH]c3n2)CC1] | 2880 |
| 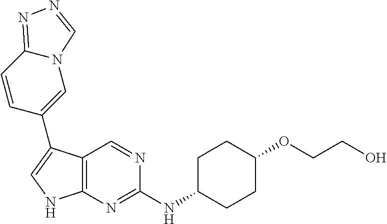 [SMILES: OCCO[C@H]1CC[C@@H](Nc2ncc3c(-c4ccc5nncn5c4)c[nH]c3n2)CC1] | 2881 |
| 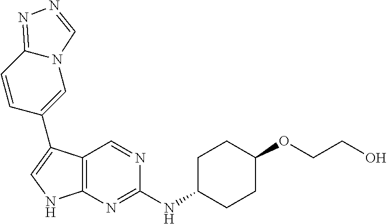 [SMILES: OCCO[C@H]1CC[C@H](Nc2ncc3c(-c4ccc5nncn5c4)c[nH]c3n2)CC1] | 2882 |
| 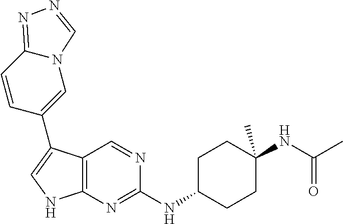 [SMILES: CC(=O)N[C@]1(C)CC[C@@H](Nc2ncc3c(-c4ccc5nncn5c4)c[nH]c3n2)CC1] | 2883 |
| 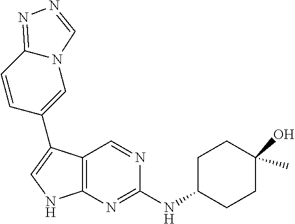 [SMILES: C[C@]1(O)CC[C@H](Nc2ncc3c(-c4ccc5nncn5c4)c[nH]c3n2)CC1] | 2884 |
| 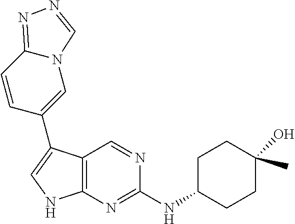 [SMILES: C[C@]1(O)CC[C@@H](Nc2ncc3c(-c4ccc5nncn5c4)c[nH]c3n2)CC1] | 2885 |
| 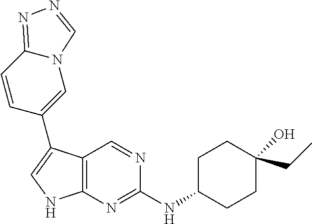 [SMILES: CC[C@]1(O)CC[C@@H](Nc2ncc3c(-c4ccc5nncn5c4)c[nH]c3n2)CC1] | 2886 |
| 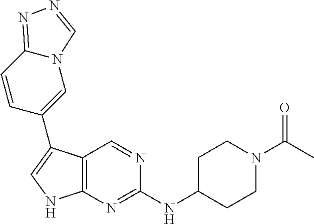 [SMILES: CC(=O)N1CCC(Nc2ncc3c(-c4ccc5nncn5c4)c[nH]c3n2)CC1] | 2887 |
| 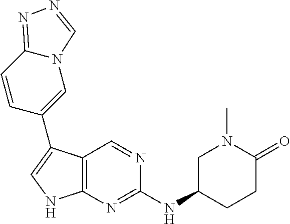 [SMILES: CN1C[C@H](Nc2ncc3c(-c4ccc5nncn5c4)c[nH]c3n2)CCC1=O] | 2888 |
| 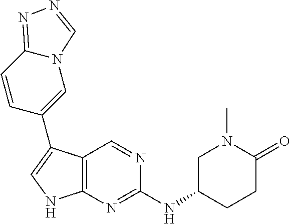 [SMILES: CN1C[C@@H](Nc2ncc3c(-c4ccc5nncn5c4)c[nH]c3n2)CCC1=O] | 2889 |
| 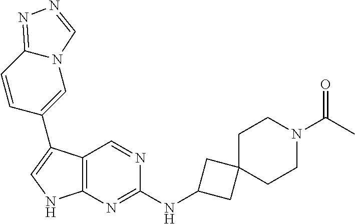 [SMILES: CC(=O)N1CCC2(CC1)CC(Nc1ncc3c(-c4ccc5nncn5c4)c[nH]c3n1)C2] | 2890 |
| 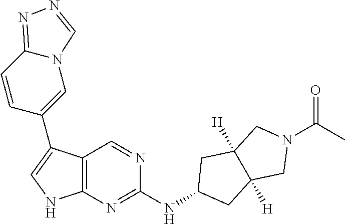 [SMILES: CC(=O)N1C[C@H]2C[C@H](Nc3ncc4c(-c5ccc6nncn6c5)c[nH]c4n3)C[C@H]2C1] | 2891 |
| 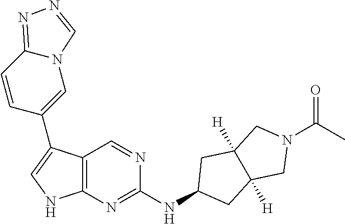 [SMILES: CC(=O)N1C[C@H]2C[C@@H](Nc3ncc4c(-c5ccc6nncn6c5)c[nH]c4n3)C[C@H]2C1] | 2892 |
| 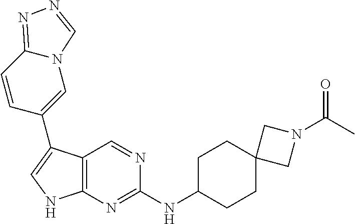 [SMILES: CC(=O)N1CC2(CCC(Nc3ncc4c(-c5ccc6nncn6c5)c[nH]c4n3)CC2)C1] | 2893 |
| 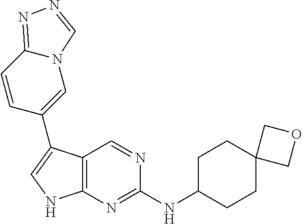 [SMILES: c1cc2nncn2cc1-c1c[nH]c2nc(NC3CCC4(CC3)COC4)ncc12] | 2894 |
| 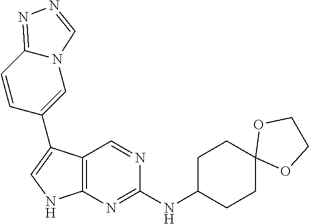 [SMILES: c1cc2nncn2cc1-c1c[nH]c2nc(NC3CCC4(CC3)OCCO4)ncc12] | 2895 |
| 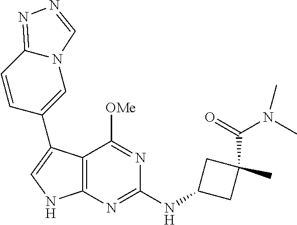 [SMILES: COc1nc(N[C@H]2C[C@@](C)(C(=O)N(C)C)C2)nc2[nH]cc(-c3ccc4nncn4c3)c12] | 2896 |
| 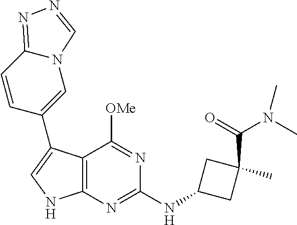 [SMILES: COc1nc(N[C@H]2C[C@](C)(C(=O)N(C)C)C2)nc2[nH]cc(-c3ccc4nncn4c3)c12] | 2897 |
| 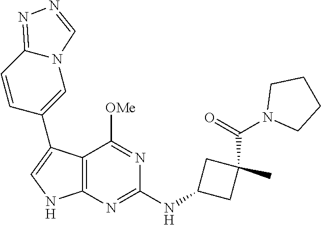 [SMILES: COc1nc(N[C@H]2C[C@@](C)(C(=O)N3CCCC3)C2)nc2[nH]cc(-c3ccc4nncn4c3)c12] | 2898 |
| 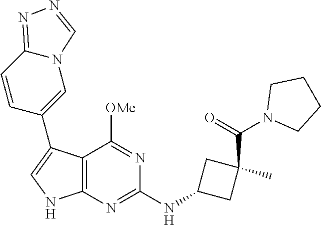 [SMILES: COc1nc(N[C@H]2C[C@](C)(C(=O)N3CCCC3)C2)nc2[nH]cc(-c3ccc4nncn4c3)c12] | 2899 |
| 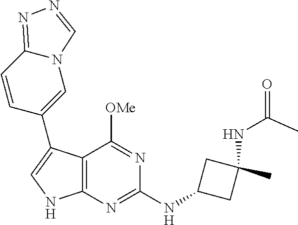 [SMILES: COc1nc(N[C@H]2C[C@@](C)(NC(C)=O)C2)nc2[nH]cc(-c3ccc4nncn4c3)c12] | 2900 |
| 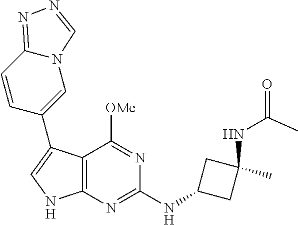 [SMILES: COc1nc(N[C@H]2C[C@](C)(NC(C)=O)C2)nc2[nH]cc(-c3ccc4nncn4c3)c12] | 2901 |
| 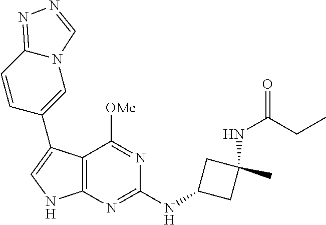 [SMILES: CCC(=O)N[C@]1(C)C[C@H](Nc2nc(OC)c3c(-c4ccc5nncn5c4)c[nH]c3n2)C1] | 2902 |
| 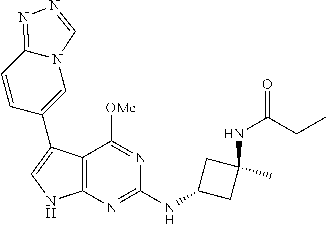 [SMILES: CCC(=O)N[C@]1(C)C[C@@H](Nc2nc(OC)c3c(-c4ccc5nncn5c4)c[nH]c3n2)C1] | 2903 |
| 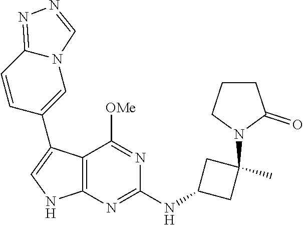 [SMILES: COc1nc(N[C@H]2C[C@](C)(N3CCCC3=O)C2)nc2[nH]cc(-c3ccc4nncn4c3)c12] | 2904 |
| 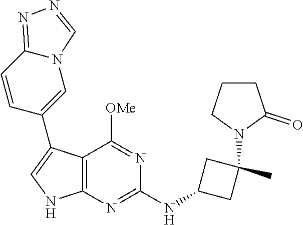 [SMILES: COc1nc(N[C@H]2C[C@@](C)(N3CCCC3=O)C2)nc2[nH]cc(-c3ccc4nncn4c3)c12] | 2905 |
| 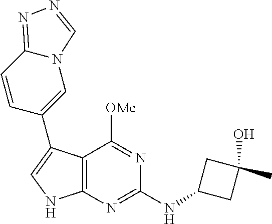 [SMILES: COc1nc(N[C@H]2C[C@@](C)(O)C2)nc2[nH]cc(-c3ccc4nncn4c3)c12] | 2906 |
| 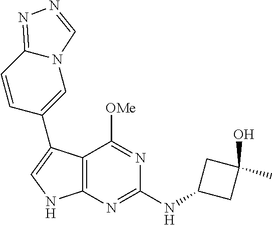 [SMILES: COc1nc(N[C@H]2C[C@](C)(O)C2)nc2[nH]cc(-c3ccc4nncn4c3)c12] | 2907 |
| 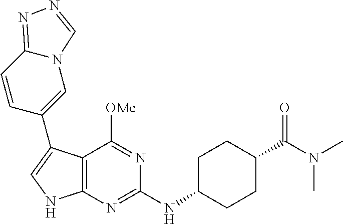 [SMILES: COc1nc(N[C@H]2CC[C@@H](C(=O)N(C)C)CC2)nc2[nH]cc(-c3ccc4nncn4c3)c12] | 2908 |
| 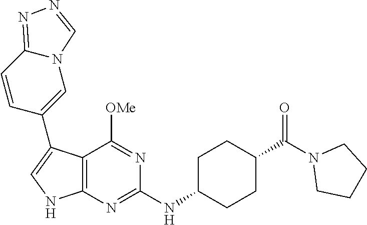 [SMILES: COc1nc(N[C@H]2CC[C@@H](C(=O)N3CCCC3)CC2)nc2[nH]cc(-c3ccc4nncn4c3)c12] | 2909 |
| 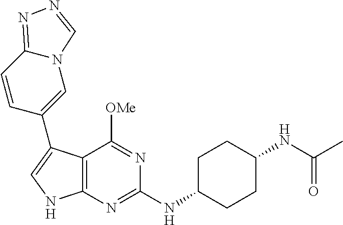 [SMILES: COc1nc(N[C@H]2CC[C@@H](NC(C)=O)CC2)nc2[nH]cc(-c3ccc4nncn4c3)c12] | 2910 |
| 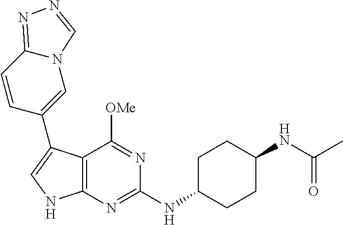 [SMILES: COc1nc(N[C@H]2CC[C@H](NC(C)=O)CC2)nc2[nH]cc(-c3ccc4nncn4c3)c12] | 2911 |
| 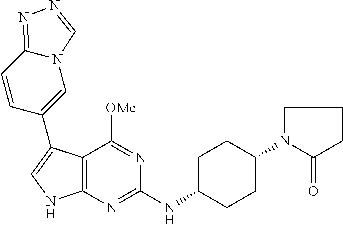 [SMILES: COc1nc(N[C@H]2CC[C@@H](N3CCCC3=O)CC2)nc2[nH]cc(-c3ccc4nncn4c3)c12] | 2912 |
| 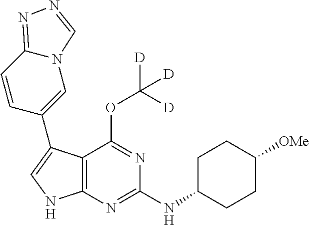 [SMILES: [2H]C([2H])([2H])Oc1nc(N[C@H]2CC[C@@H](OC)CC2)nc2[nH]cc(-c3ccc4nncn4c3)c12] | 2913 |
| 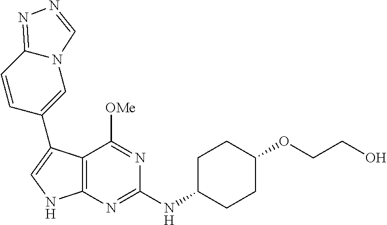 [SMILES: COc1nc(N[C@H]2CC[C@@H](OCCO)CC2)nc2[nH]cc(-c3ccc4nncn4c3)c12] | 2914 |
| 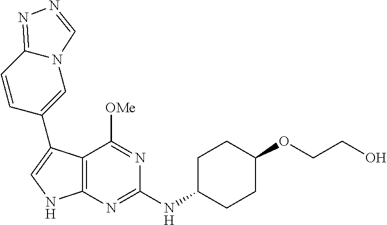 [SMILES: COc1nc(N[C@H]2CC[C@H](OCCO)CC2)nc2[nH]cc(-c3ccc4nncn4c3)c12] | 2915 |
| 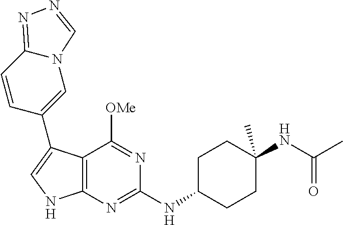 [SMILES: COc1nc(N[C@H]2CC[C@](C)(NC(C)=O)CC2)nc2[nH]cc(-c3ccc4nncn4c3)c12] | 2916 |
| 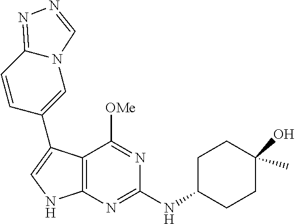 [SMILES: COc1nc(N[C@H]2CC[C@](C)(O)CC2)nc2[nH]cc(-c3ccc4nncn4c3)c12] | 2917 |
| 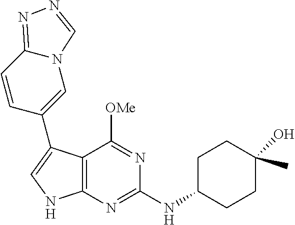 [SMILES: COc1nc(N[C@H]2CC[C@@](C)(O)CC2)nc2[nH]cc(-c3ccc4nncn4c3)c12] | 2918 |
| 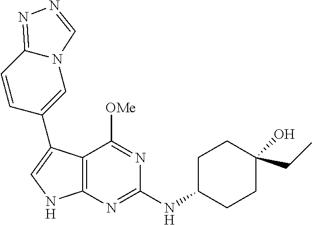 [SMILES: CC[C@]1(O)CC[C@@H](Nc2nc(OC)c3c(-c4ccc5nncn5c4)c[nH]c3n2)CC1] | 2919 |
| 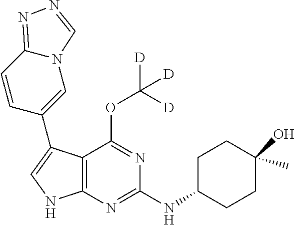 [SMILES: [2H]C([2H])([2H])Oc1nc(N[C@H]2CC[C@](C)(O)CC2)nc2[nH]cc(-c3ccc4nncn4c3)c12] | 2920 |
| 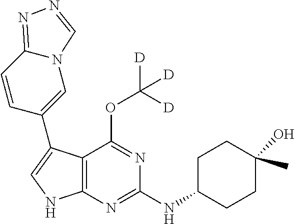 [SMILES: [2H]C([2H])([2H])Oc1nc(N[C@H]2CC[C@@](C)(O)CC2)nc2[nH]cc(-c3ccc4nncn4c3)c12] | 2921 |
| 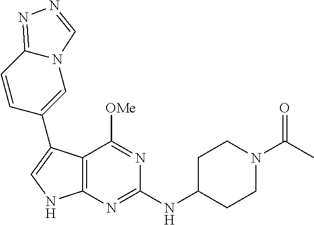 [SMILES: COc1nc(NC2CCN(C(C)=O)CC2)nc2[nH]cc(-c3ccc4nncn4c3)c12] | 2922 |
| 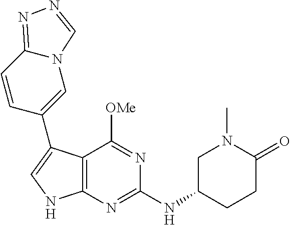 [SMILES: COc1nc(N[C@H]2CCC(=O)N(C)C2)nc2[nH]cc(-c3ccc4nncn4c3)c12] | 2923 |
| 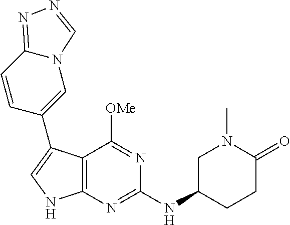 [SMILES: COc1nc(N[C@@H]2CCC(=O)N(C)C2)nc2[nH]cc(-c3ccc4nncn4c3)c12] | 2924 |
| 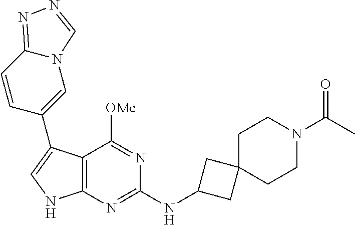 [SMILES: COc1nc(NC2CC3(CCN(C(C)=O)CC3)C2)nc2[nH]cc(-c3ccc4nncn4c3)c12] | 2925 |
| 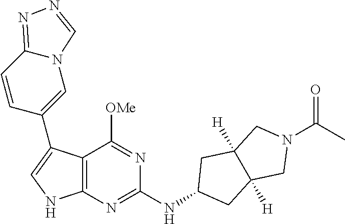 [SMILES: COc1nc(N[C@H]2C[C@@H]3CN(C(C)=O)C[C@@H]3C2)nc2[nH]cc(-c3ccc4nncn4c3)c12] | 2926 |
| 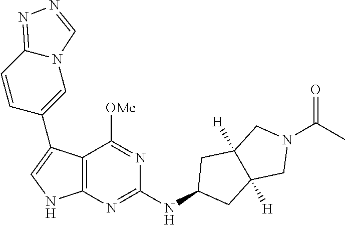 [SMILES: COc1nc(N[C@@H]2C[C@@H]3CN(C(C)=O)C[C@@H]3C2)nc2[nH]cc(-c3ccc4nncn4c3)c12] | 2927 |
| 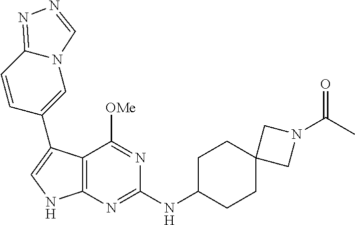 [SMILES: COc1nc(NC2CCC3(CC2)CN(C(C)=O)C3)nc2[nH]cc(-c3ccc4nncn4c3)c12] | 2928 |
| 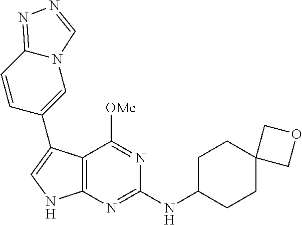 [SMILES: COc1nc(NC2CCC3(CC2)COC3)nc2[nH]cc(-c3ccc4nncn4c3)c12] | 2929 |
| 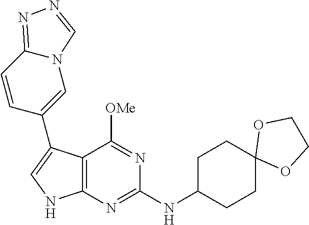 [SMILES: COc1nc(NC2CCC3(CC2)OCCO3)nc2[nH]cc(-c3ccc4nncn4c3)c12] | 2930 |
| 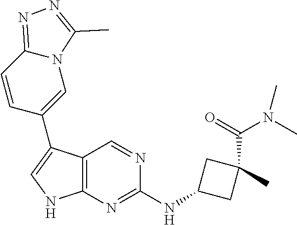 [SMILES: Cc1nnc2ccc(-c3c[nH]c4nc(N[C@H]5C[C@@](C)(C(=O)N(C)C)C5)ncc34)cn12] | 2931 |
| 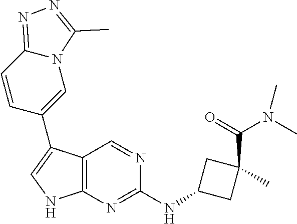 [SMILES: Cc1nnc2ccc(-c3c[nH]c4nc(N[C@H]5C[C@](C)(C(=O)N(C)C)C5)ncc34)cn12] | 2932 |
| 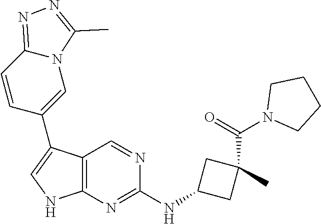 [SMILES: Cc1nnc2ccc(-c3c[nH]c4nc(N[C@H]5C[C@@](C)(C(=O)N6CCCC6)C5)ncc34)cn12] | 2933 |
| 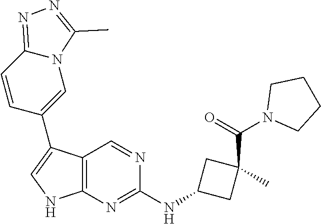 [SMILES: Cc1nnc2ccc(-c3c[nH]c4nc(N[C@H]5C[C@](C)(C(=O)N6CCCC6)C5)ncc34)cn12] | 2934 |
| 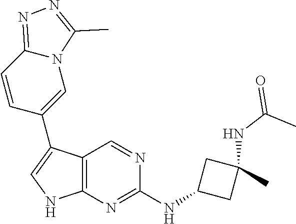 [SMILES: CC(=O)N[C@]1(C)C[C@H](Nc2ncc3c(-c4ccc5nnc(C)n5c4)c[nH]c3n2)C1] | 2935 |
| 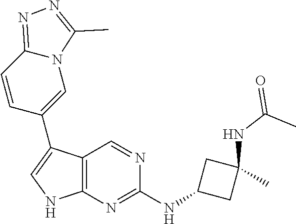 [SMILES: CC(=O)N[C@]1(C)C[C@@H](Nc2ncc3c(-c4ccc5nnc(C)n5c4)c[nH]c3n2)C1] | 2936 |
| 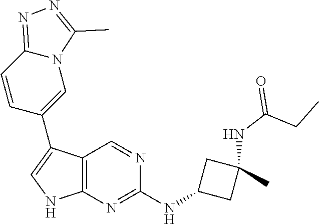 [SMILES: CCC(=O)N[C@]1(C)C[C@H](Nc2ncc3c(-c4ccc5nnc(C)n5c4)c[nH]c3n2)C1] | 2937 |
| 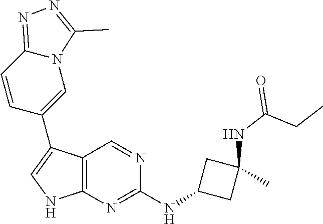 [SMILES: CCC(=O)N[C@]1(C)C[C@@H](Nc2ncc3c(-c4ccc5nnc(C)n5c4)c[nH]c3n2)C1] | 2938 |
| 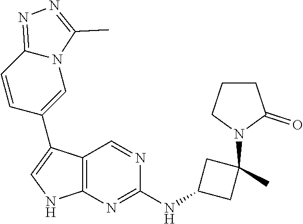 [SMILES: Cc1nnc2ccc(-c3c[nH]c4nc(N[C@H]5C[C@](C)(N6CCCC6=O)C5)ncc34)cn12] | 2939 |
| 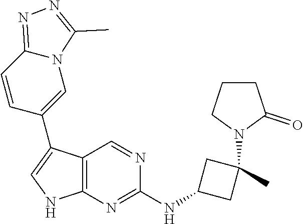 [SMILES: Cc1nnc2ccc(-c3c[nH]c4nc(N[C@H]5C[C@@](C)(N6CCCC6=O)C5)ncc34)cn12] | 2940 |
| 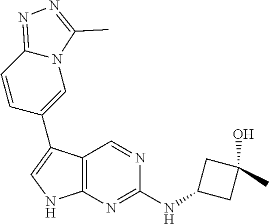 [SMILES: Cc1nnc2ccc(-c3c[nH]c4nc(N[C@H]5C[C@@](C)(O)C5)ncc34)cn12] | 2941 |
| 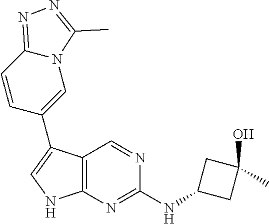 [SMILES: Cc1nnc2ccc(-c3c[nH]c4nc(N[C@H]5C[C@](C)(O)C5)ncc34)cn12] | 2942 |
| 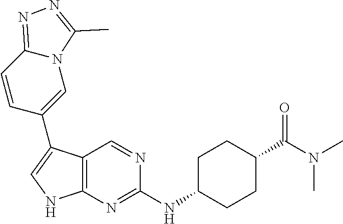 [SMILES: Cc1nnc2ccc(-c3c[nH]c4nc(N[C@H]5CC[C@@H](C(=O)N(C)C)CC5)ncc34)cn12] | 2943 |
| 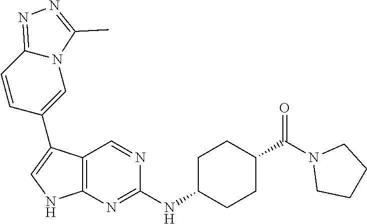 [SMILES: Cc1nnc2ccc(-c3c[nH]c4nc(N[C@H]5CC[C@@H](C(=O)N6CCCC6)CC5)ncc34)cn12] | 2944 |
| 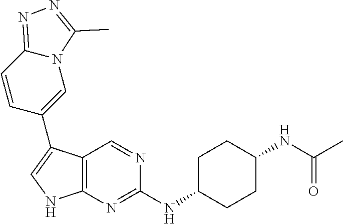 [SMILES: CC(=O)N[C@H]1CC[C@@H](Nc2ncc3c(-c4ccc5nnc(C)n5c4)c[nH]c3n2)CC1] | 2945 |
| 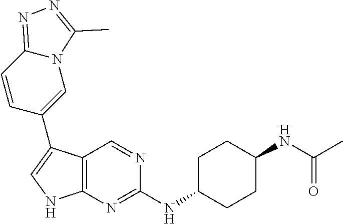 [SMILES: CC(=O)N[C@H]1CC[C@H](Nc2ncc3c(-c4ccc5nnc(C)n5c4)c[nH]c3n2)CC1] | 2946 |
| 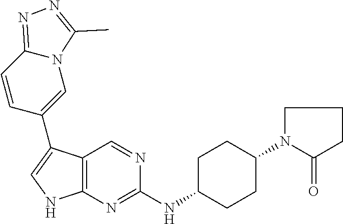 [SMILES: Cc1nnc2ccc(-c3c[nH]c4nc(N[C@H]5CC[C@@H](N6CCCC6=O)CC5)ncc34)cn12] | 2947 |
| 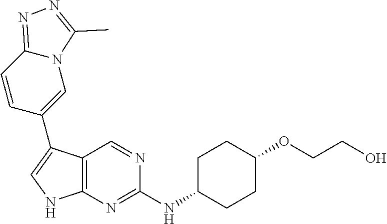 [SMILES: Cc1nnc2ccc(-c3c[nH]c4nc(N[C@H]5CC[C@@H](OCCO)CC5)ncc34)cn12] | 2948 |
| 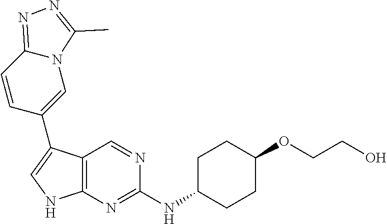 [SMILES: Cc1nnc2ccc(-c3c[nH]c4nc(N[C@H]5CC[C@H](OCCO)CC5)ncc34)cn12] | 2949 |
| 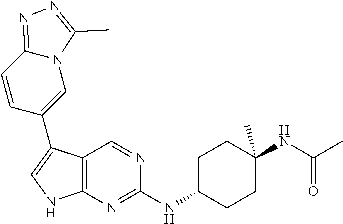 [SMILES: CC(=O)N[C@]1(C)CC[C@@H](Nc2ncc3c(-c4ccc5nnc(C)n5c4)c[nH]c3n2)CC1] | 2950 |
| 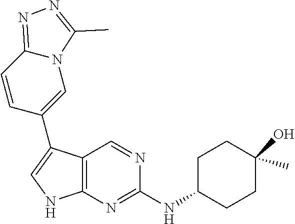 [SMILES: Cc1nnc2ccc(-c3c[nH]c4nc(N[C@H]5CC[C@](C)(O)CC5)ncc34)cn12] | 2951 |
| 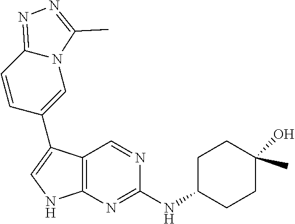 [SMILES: Cc1nnc2ccc(-c3c[nH]c4nc(N[C@H]5CC[C@@](C)(O)CC5)ncc34)cn12] | 2952 |
| 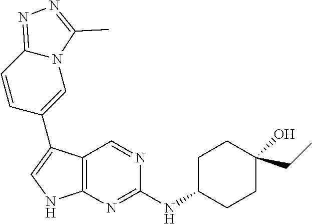 [SMILES: CC[C@]1(O)CC[C@@H](Nc2ncc3c(-c4ccc5nnc(C)n5c4)c[nH]c3n2)CC1] | 2953 |
| 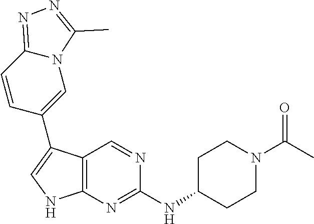 | 2954 |
| 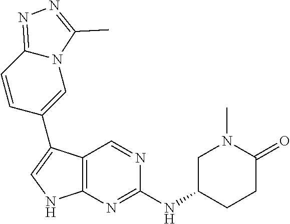 [SMILES: Cc1nnc2ccc(-c3c[nH]c4nc(N[C@H]5CCC(=O)N(C)C5)ncc34)cn12] | 2955 |
| 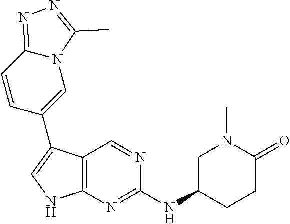 [SMILES: Cc1nnc2ccc(-c3c[nH]c4nc(N[C@@H]5CCC(=O)N(C)C5)ncc34)cn12] | 2956 |
| 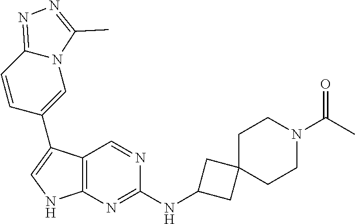 [SMILES: CC(=O)N1CCC2(CC1)CC(Nc1ncc3c(-c4ccc5nnc(C)n5c4)c[nH]c3n1)C2] | 2957 |
| 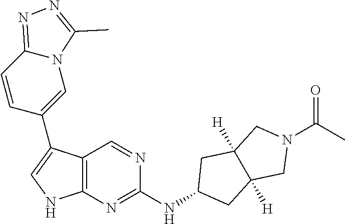 [SMILES: CC(=O)N1C[C@H]2C[C@H](Nc3ncc4c(-c5ccc6nnc(C)n6c5)c[nH]c4n3)C[C@H]2C1] | 2958 |
| 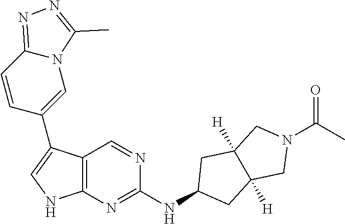 [SMILES: CC(=O)N1C[C@H]2C[C@@H](Nc3ncc4c(-c5ccc6nnc(C)n6c5)c[nH]c4n3)C[C@H]2C1] | 2959 |
| 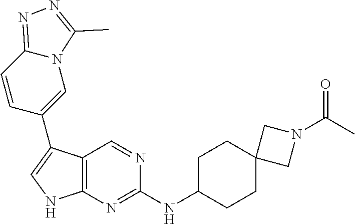 [SMILES: CC(=O)N1CC2(CCC(Nc3ncc4c(-c5ccc6nnc(C)n6c5)c[nH]c4n3)CC2)C1] | 2960 |
| 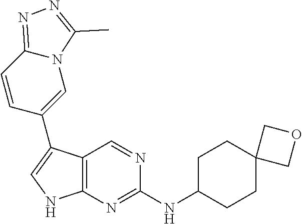 [SMILES: Cc1nnc2ccc(-c3c[nH]c4nc(NC5CCC6(CC5)COC6)ncc34)cn12] | 2961 |
| 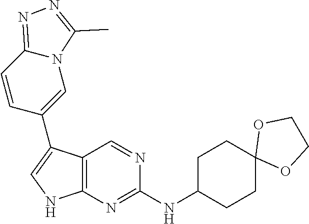 [SMILES: Cc1nnc2ccc(-c3c[nH]c4nc(NC5CCC6(CC5)OCCO6)ncc34)cn12] | 2962 |
| 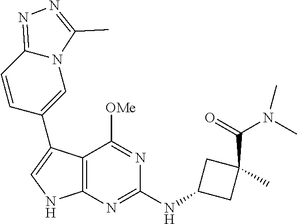 [SMILES: COc1nc(N[C@H]2C[C@](C)(C(=O)N(C)C)C2)nc2[nH]cc(-c3ccc4nnc(C)n4c3)c12] | 2963 |
| 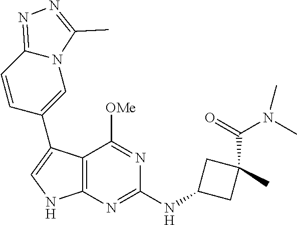 [SMILES: COc1nc(N[C@H]2C[C@@](C)(C(=O)N(C)C)C2)nc2[nH]cc(-c3ccc4nnc(C)n4c3)c12] | 2964 |
| 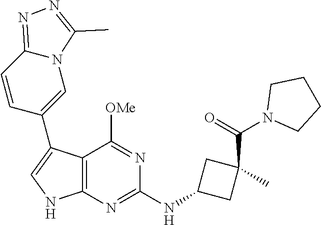 [SMILES: COc1nc(N[C@H]2C[C@](C)(C(=O)N3CCCC3)C2)nc2[nH]cc(-c3ccc4nnc(C)n4c3)c12] | 2965 |
| 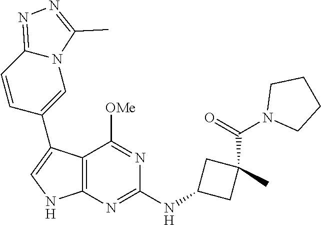 [SMILES: COc1nc(N[C@H]2C[C@@](C)(C(=O)N3CCCC3)C2)nc2[nH]cc(-c3ccc4nnc(C)n4c3)c12] | 2966 |
| 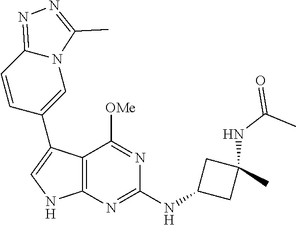 [SMILES: COc1nc(N[C@H]2C[C@@](C)(NC(C)=O)C2)nc2[nH]cc(-c3ccc4nnc(C)n4c3)c12] | 2967 |
| 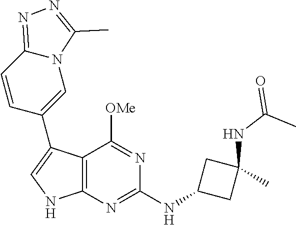 [SMILES: COc1nc(N[C@H]2C[C@](C)(NC(C)=O)C2)nc2[nH]cc(-c3ccc4nnc(C)n4c3)c12] | 2968 |
| 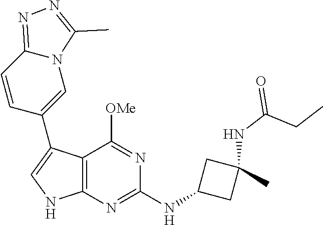 [SMILES: CCC(=O)N[C@]1(C)C[C@H](Nc2nc(OC)c3c(-c4ccc5nnc(C)n5c4)c[nH]c3n2)C1] | 2969 |
| 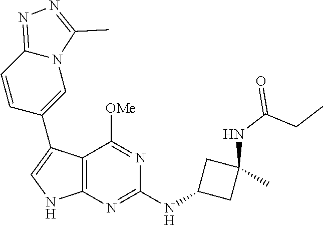 [SMILES: CCC(=O)N[C@]1(C)C[C@@H](Nc2nc(OC)c3c(-c4ccc5nnc(C)n5c4)c[nH]c3n2)C1] | 2970 |
| 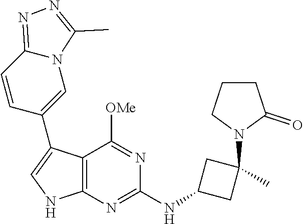 [SMILES: COc1nc(N[C@H]2C[C@](C)(N3CCCC3=O)C2)nc2[nH]cc(-c3ccc4nnc(C)n4c3)c12] | 2971 |
| 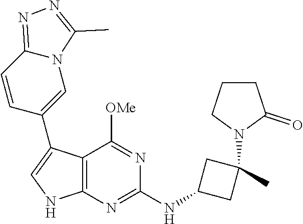 [SMILES: COc1nc(N[C@H]2C[C@@](C)(N3CCCC3=O)C2)nc2[nH]cc(-c3ccc4nnc(C)n4c3)c12] | 2972 |
| 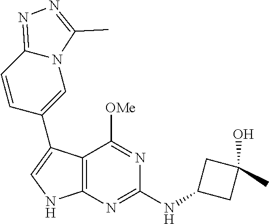 [SMILES: COc1nc(N[C@H]2C[C@@](C)(O)C2)nc2[nH]cc(-c3ccc4nnc(C)n4c3)c12] | 2973 |
| 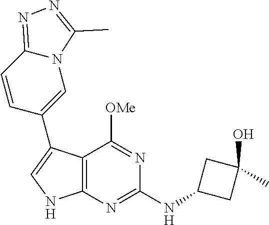 [SMILES: COc1nc(N[C@H]2C[C@](C)(O)C2)nc2[nH]cc(-c3ccc4nnc(C)n4c3)c12] | 2974 |
| 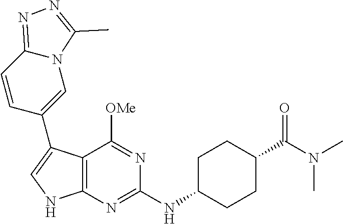 [SMILES: COc1nc(N[C@H]2CC[C@@H](C(=O)N(C)C)CC2)nc2[nH]cc(-c3ccc4nnc(C)n4c3)c12] | 2975 |
| 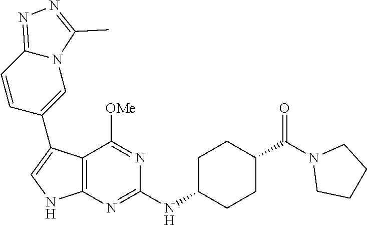 [SMILES: COc1nc(N[C@H]2CC[C@@H](C(=O)N3CCCC3)CC2)nc2[nH]cc(-c3ccc4nnc(C)n4c3)c12] | 2976 |
| 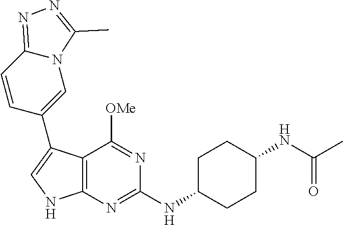 [SMILES: COc1nc(N[C@H]2CC[C@@H](NC(C)=O)CC2)nc2[nH]cc(-c3ccc4nnc(C)n4c3)c12] | 2977 |
| 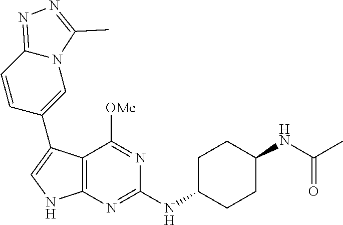 [SMILES: COc1nc(N[C@H]2CC[C@H](NC(C)=O)CC2)nc2[nH]cc(-c3ccc4nnc(C)n4c3)c12] | 2978 |
| 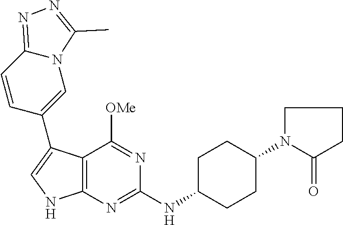 [SMILES: COc1nc(N[C@H]2CC[C@@H](N3CCCC3=O)CC2)nc2[nH]cc(-c3ccc4nnc(C)n4c3)c12] | 2979 |
| 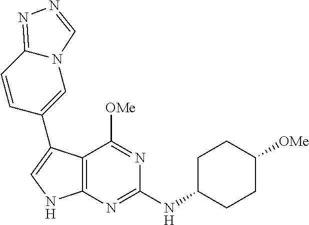 [SMILES: COc1nc(N[C@H]2CC[C@@H](OC)CC2)nc2[nH]cc(-c3ccc4nncn4c3)c12] | 2980 |
| 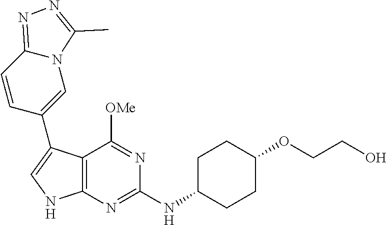 [SMILES: COc1nc(N[C@H]2CC[C@@H](OCCO)CC2)nc2[nH]cc(-c3ccc4nnc(C)n4c3)c12] | 2981 |
| 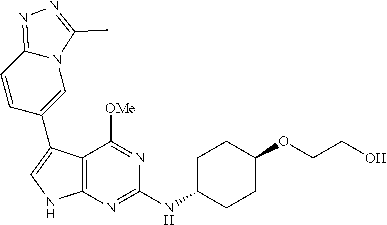 [SMILES: COc1nc(N[C@H]2CC[C@H](OCCO)CC2)nc2[nH]cc(-c3ccc4nnc(C)n4c3)c12] | 2982 |
| 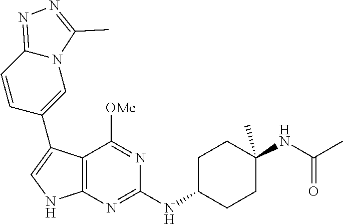 [SMILES: COc1nc(N[C@H]2CC[C@](C)(NC(C)=O)CC2)nc2[nH]cc(-c3ccc4nnc(C)n4c3)c12] | 2983 |
| 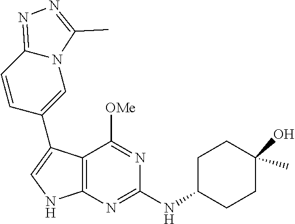 [SMILES: COc1nc(N[C@H]2CC[C@](C)(O)CC2)nc2[nH]cc(-c3ccc4nnc(C)n4c3)c12] | 2984 |
| 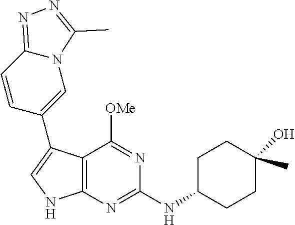 [SMILES: COc1nc(N[C@H]2CC[C@@](C)(O)CC2)nc2[nH]cc(-c3ccc4nnc(C)n4c3)c12] | 2985 |
| 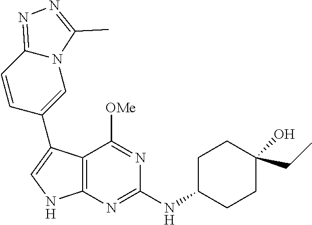 [SMILES: CC[C@]1(O)CC[C@@H](Nc2nc(OC)c3c(-c4ccc5nnc(C)n5c4)c[nH]c3n2)CC1] | 2986 |
| 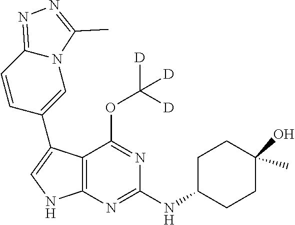 [SMILES: [2H]C([2H])([2H])Oc1nc(N[C@H]2CC[C@](C)(O)CC2)nc2[nH]cc(-c3ccc4nnc(C)n4c3)c12] | 2987 |
| 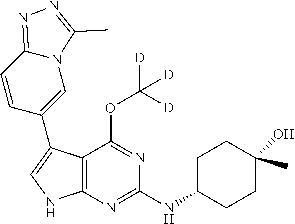 [SMILES: [2H]C([2H])([2H])Oc1nc(N[C@H]2CC[C@@](C)(O)CC2)nc2[nH]cc(-c3ccc4nnc(C)n4c3)c12] | 2988 |
| 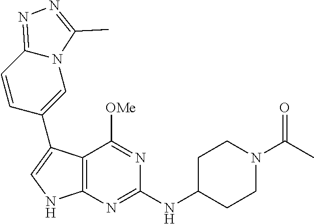 [SMILES: COc1nc(NC2CCN(C(C)=O)CC2)nc2[nH]cc(-c3ccc4nnc(C)n4c3)c12] | 2989 |
| 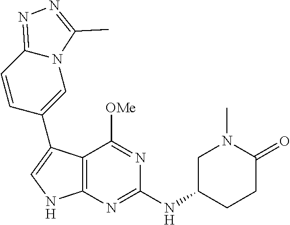 [SMILES: COc1nc(N[C@H]2CCC(=O)N(C)C2)nc2[nH]cc(-c3ccc4nnc(C)n4c3)c12] | 2990 |
| 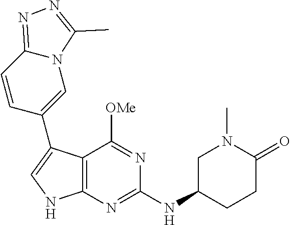 [SMILES: COc1nc(N[C@@H]2CCC(=O)N(C)C2)nc2[nH]cc(-c3ccc4nnc(C)n4c3)c12] | 2991 |
| 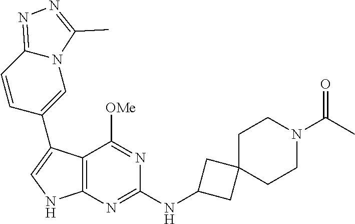 [SMILES: COc1nc(NC2CC3(CCN(C(C)=O)CC3)C2)nc2[nH]cc(-c3ccc4nnc(C)n4c3)c12] | 2992 |
| 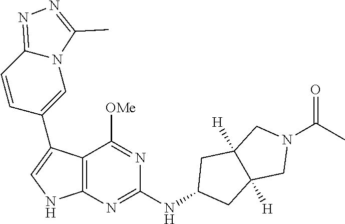 [SMILES: COc1nc(N[C@H]2C[C@@H]3CN(C(C)=O)C[C@@H]3C2)nc2[nH]cc(-c3ccc4nnc(C)n4c3)c12] | 2993 |
| 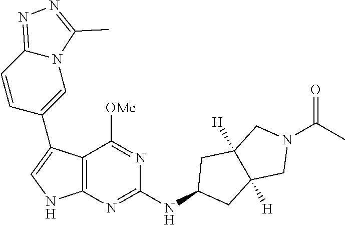 [SMILES: COc1nc(N[C@@H]2C[C@@H]3CN(C(C)=O)C[C@@H]3C2)nc2[nH]cc(-c3ccc4nnc(C)n4c3)c12] | 2994 |
| 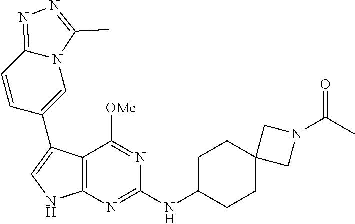 [SMILES: COc1nc(NC2CCC3(CC2)CN(C(C)=O)C3)nc2[nH]cc(-c3ccc4nnc(C)n4c3)c12] | 2995 |
| 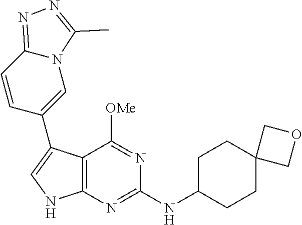 [SMILES: COc1nc(NC2CCC3(CC2)COC3)nc2[nH]cc(-c3ccc4nnc(C)n4c3)c12] | 2996 |
| 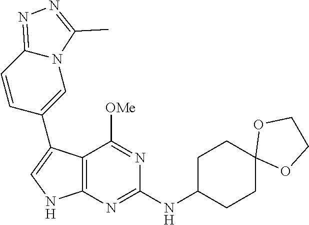 [SMILES: COc1nc(NC2CCC3(CC2)OCCO3)nc2[nH]cc(-c3ccc4nnc(C)n4c3)c12] | 2997 |
| 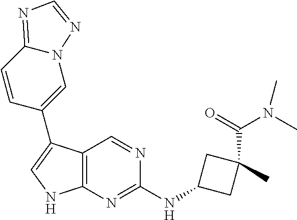 [SMILES: CN(C)C(=O)[C@]1(C)C[C@H](Nc2ncc3c(-c4ccc5ncnn5c4)c[nH]c3n2)C1] | 2998 |
| 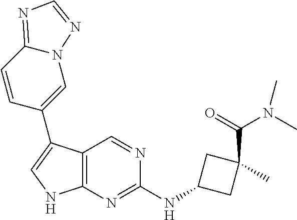 [SMILES: CN(C)C(=O)[C@]1(C)C[C@@H](Nc2ncc3c(-c4ccc5ncnn5c4)c[nH]c3n2)C1] | 2999 |
| 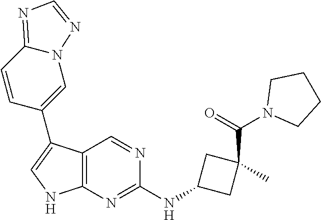 [SMILES: C[C@]1(C(=O)N2CCCC2)C[C@H](Nc2ncc3c(-c4ccc5ncnn5c4)c[nH]c3n2)C1] | 3000 |
| 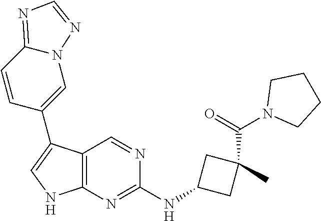 [SMILES: C[C@]1(C(=O)N2CCCC2)C[C@@H](Nc2ncc3c(-c4ccc5ncnn5c4)c[nH]c3n2)C1] | 3001 |
| 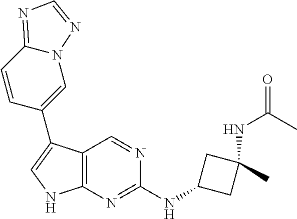 [SMILES: CC(=O)N[C@]1(C)C[C@H](Nc2ncc3c(-c4ccc5ncnn5c4)c[nH]c3n2)C1] | 3002 |
| 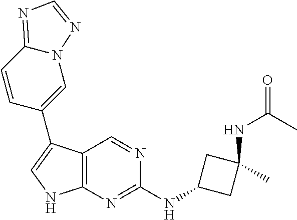 [SMILES: CC(=O)N[C@]1(C)C[C@@H](Nc2ncc3c(-c4ccc5ncnn5c4)c[nH]c3n2)C1] | 3003 |
| 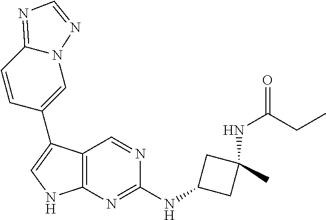 [SMILES: CCC(=O)N[C@]1(C)C[C@H](Nc2ncc3c(-c4ccc5ncnn5c4)c[nH]c3n2)C1] | 3004 |
| 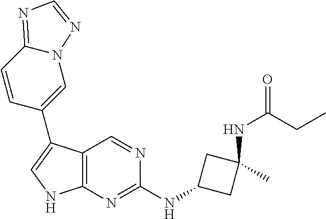 [SMILES: CCC(=O)N[C@]1(C)C[C@@H](Nc2ncc3c(-c4ccc5ncnn5c4)c[nH]c3n2)C1] | 3005 |
| 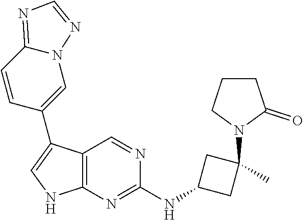 [SMILES: C[C@]1(N2CCCC2=O)C[C@H](Nc2ncc3c(-c4ccc5ncnn5c4)c[nH]c3n2)C1] | 3006 |
| 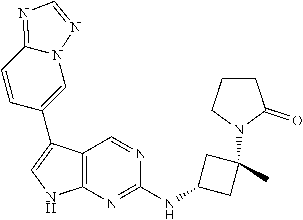 [SMILES: C[C@]1(N2CCCC2=O)C[C@@H](Nc2ncc3c(-c4ccc5ncnn5c4)c[nH]c3n2)C1] | 3007 |
| 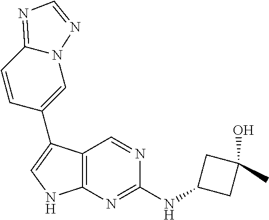 [SMILES: C[C@]1(O)C[C@@H](Nc2ncc3c(-c4ccc5ncnn5c4)c[nH]c3n2)C1] | 3008 |
| 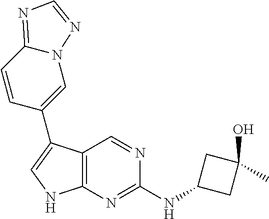 [SMILES: C[C@]1(O)C[C@H](Nc2ncc3c(-c4ccc5ncnn5c4)c[nH]c3n2)C1] | 3009 |
| 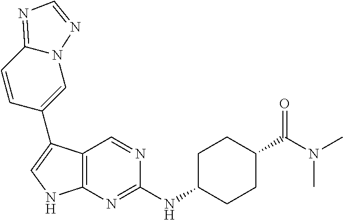 [SMILES: CN(C)C(=O)[C@H]1CC[C@@H](Nc2ncc3c(-c4ccc5ncnn5c4)c[nH]c3n2)CC1] | 3010 |
| 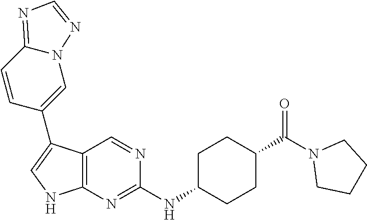 [SMILES: O=C([C@H]1CC[C@@H](Nc2ncc3c(-c4ccc5ncnn5c4)c[nH]c3n2)CC1)N1CCCC1] | 3011 |
| 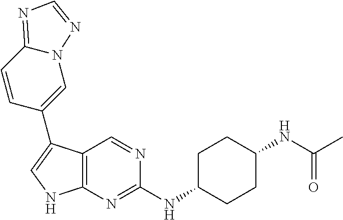 [SMILES: CC(=O)N[C@H]1CC[C@@H](Nc2ncc3c(-c4ccc5ncnn5c4)c[nH]c3n2)CC1] | 3012 |
| 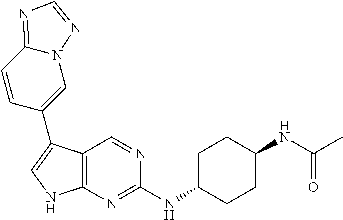 [SMILES: CC(=O)N[C@H]1CC[C@H](Nc2ncc3c(-c4ccc5ncnn5c4)c[nH]c3n2)CC1] | 3013 |
| 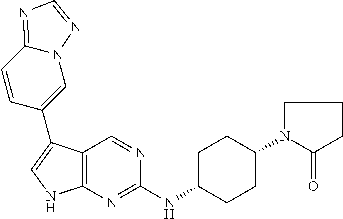 [SMILES: O=C1CCCN1[C@H]1CC[C@@H](Nc2ncc3c(-c4ccc5ncnn5c4)c[nH]c3n2)CC1] | 3014 |
| 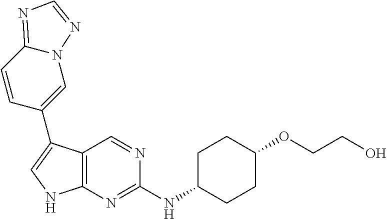 [SMILES: OCCO[C@H]1CC[C@@H](Nc2ncc3c(-c4ccc5ncnn5c4)c[nH]c3n2)CC1] | 3015 |
| 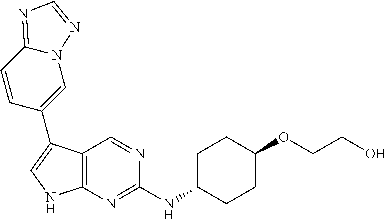 [SMILES: OCCO[C@H]1CC[C@H](Nc2ncc3c(-c4ccc5ncnn5c4)c[nH]c3n2)CC1] | 3016 |
| 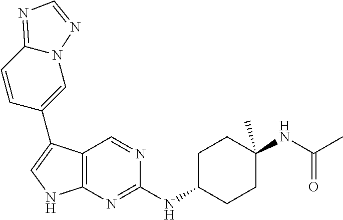 [SMILES: CC(=O)N[C@]1(C)CC[C@@H](Nc2ncc3c(-c4ccc5ncnn5c4)c[nH]c3n2)CC1] | 3017 |
| 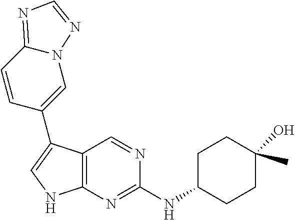 [SMILES: C[C@]1(O)CC[C@@H](Nc2ncc3c(-c4ccc5ncnn5c4)c[nH]c3n2)CC1] | 3018 |
| 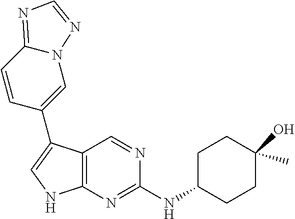 [SMILES: C[C@]1(O)CC[C@H](Nc2ncc3c(-c4ccc5ncnn5c4)c[nH]c3n2)CC1] | 3019 |
| 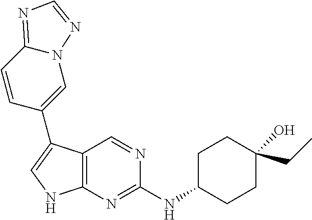 [SMILES: CC[C@]1(O)CC[C@@H](Nc2ncc3c(-c4ccc5ncnn5c4)c[nH]c3n2)CC1] | 3020 |
| 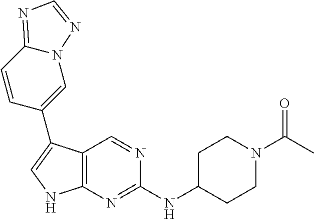 [SMILES: CC(=O)N1CCC(Nc2ncc3c(-c4ccc5ncnn5c4)c[nH]c3n2)CC1] | 3021 |
| 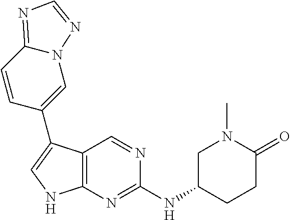 [SMILES: CN1C[C@@H](Nc2ncc3c(-c4ccc5ncnn5c4)c[nH]c3n2)CCC1=O] | 3022 |
| 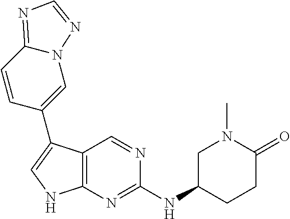 [SMILES: CN1C[C@H](Nc2ncc3c(-c4ccc5ncnn5c4)c[nH]c3n2)CCC1=O] | 3023 |
| 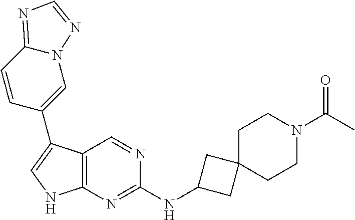 [SMILES: CC(=O)N1CCC2(CC1)CC(Nc1ncc3c(-c4ccc5ncnn5c4)c[nH]c3n1)C2] | 3024 |
| 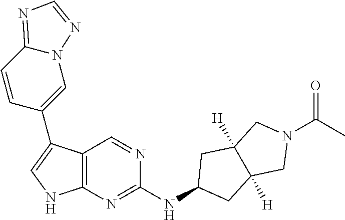 [SMILES: CC(=O)N1C[C@H]2C[C@@H](Nc3ncc4c(-c5ccc6ncnn6c5)c[nH]c4n3)C[C@H]2C1] | 3025 |
| 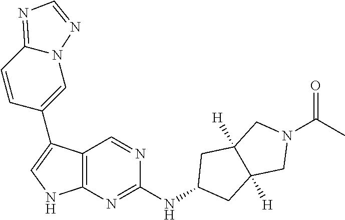 [SMILES: CC(=O)N1C[C@H]2C[C@H](Nc3ncc4c(-c5ccc6ncnn6c5)c[nH]c4n3)C[C@H]2C1] | 3026 |
| 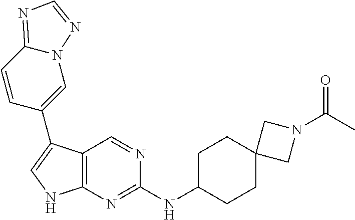 [SMILES: CC(=O)N1CC2(CCC(Nc3ncc4c(-c5ccc6ncnn6c5)c[nH]c4n3)CC2)C1] | 3027 |
| 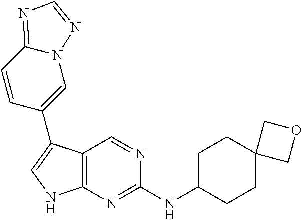 [SMILES: c1nc2ccc(-c3c[nH]c4nc(NC5CCC6(CC5)COC6)ncc34)cn2n1] | 3028 |
| 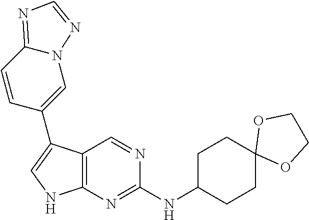 [SMILES: c1nc2ccc(-c3c[nH]c4nc(NC5CCC6(CC5)OCCO6)ncc34)cn2n1] | 3029 |
| 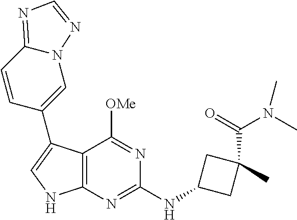 [SMILES: COc1nc(N[C@H]2C[C@@](C)(C(=O)N(C)C)C2)nc2[nH]cc(-c3ccc4ncnn4c3)c12] | 3030 |
| 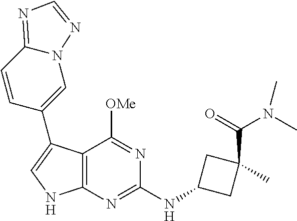 [SMILES: COc1nc(N[C@H]2C[C@](C)(C(=O)N(C)C)C2)nc2[nH]cc(-c3ccc4ncnn4c3)c12] | 3031 |
| 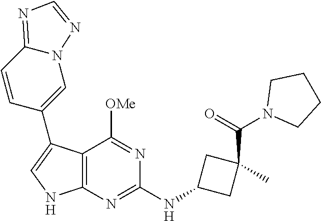 [SMILES: COc1nc(N[C@H]2C[C@](C)(C(=O)N3CCCC3)C2)nc2[nH]cc(-c3ccc4ncnn4c3)c12] | 3032 |
| 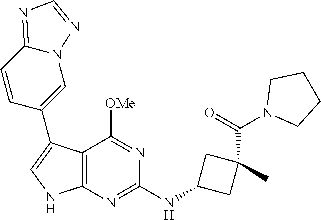 [SMILES: COc1nc(N[C@H]2C[C@@](C)(C(=O)N3CCCC3)C2)nc2[nH]cc(-c3ccc4ncnn4c3)c12] | 3033 |
| 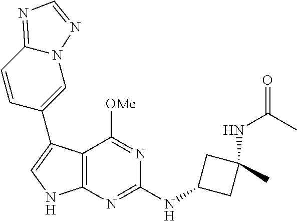 [SMILES: COc1nc(N[C@H]2C[C@@](C)(NC(C)=O)C2)nc2[nH]cc(-c3ccc4ncnn4c3)c12] | 3034 |
| 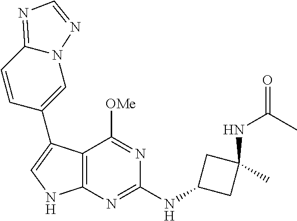 [SMILES: COc1nc(N[C@H]2C[C@](C)(NC(C)=O)C2)nc2[nH]cc(-c3ccc4ncnn4c3)c12] | 3035 |
| 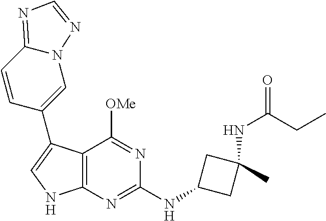 [SMILES: CCC(=O)N[C@]1(C)C[C@H](Nc2nc(OC)c3c(-c4ccc5ncnn5c4)c[nH]c3n2)C1] | 3036 |
| 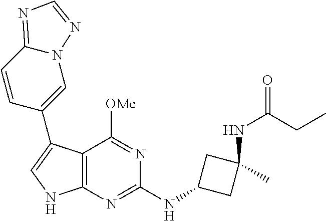 [SMILES: CCC(=O)N[C@]1(C)C[C@@H](Nc2nc(OC)c3c(-c4ccc5ncnn5c4)c[nH]c3n2)C1] | 3037 |
| 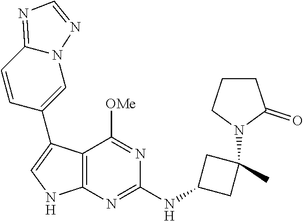 [SMILES: COc1nc(N[C@H]2C[C@@](C)(N3CCCC3=O)C2)nc2[nH]cc(-c3ccc4ncnn4c3)c12] | 3038 |
| 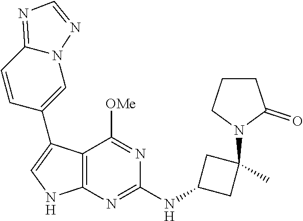 [SMILES: COc1nc(N[C@H]2C[C@](C)(N3CCCC3=O)C2)nc2[nH]cc(-c3ccc4ncnn4c3)c12] | 3039 |
| 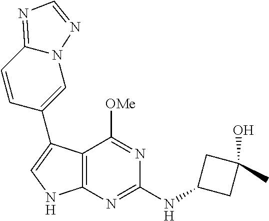 [SMILES: COc1nc(N[C@H]2C[C@@](C)(O)C2)nc2[nH]cc(-c3ccc4ncnn4c3)c12] | 3040 |
| 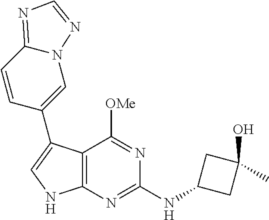 [SMILES: COc1nc(N[C@H]2C[C@](C)(O)C2)nc2[nH]cc(-c3ccc4ncnn4c3)c12] | 3041 |
| 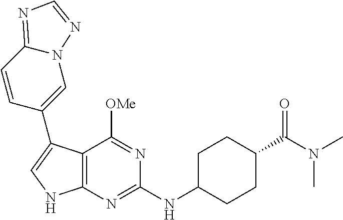 | 3042 |
| 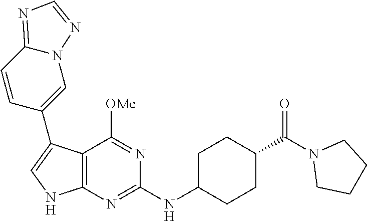 | 3043 |
| 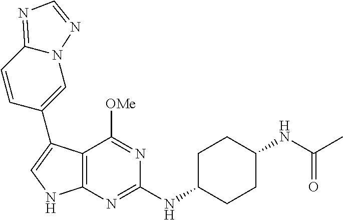 [SMILES: COc1nc(N[C@H]2CC[C@@H](NC(C)=O)CC2)nc2[nH]cc(-c3ccc4ncnn4c3)c12] | 3044 |
| 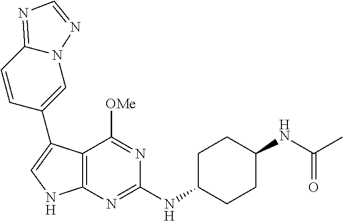 [SMILES: COc1nc(N[C@H]2CC[C@H](NC(C)=O)CC2)nc2[nH]cc(-c3ccc4ncnn4c3)c12] | 3045 |
| 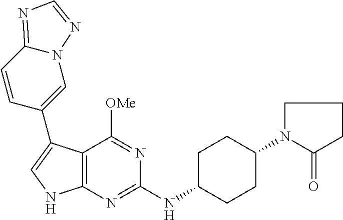 [SMILES: COc1nc(N[C@H]2CC[C@@H](N3CCCC3=O)CC2)nc2[nH]cc(-c3ccc4ncnn4c3)c12] | 3046 |
| 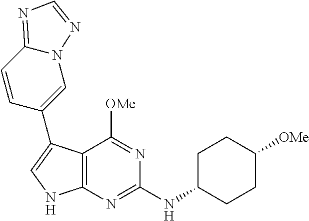 [SMILES: COc1nc(N[C@H]2CC[C@@H](OC)CC2)nc2[nH]cc(-c3ccc4ncnn4c3)c12] | 3047 |
| 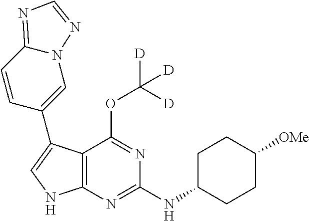 [SMILES: [2H]C([2H])([2H])Oc1nc(N[C@H]2CC[C@@H](OC)CC2)nc2[nH]cc(-c3ccc4ncnn4c3)c12] | 3048 |
| 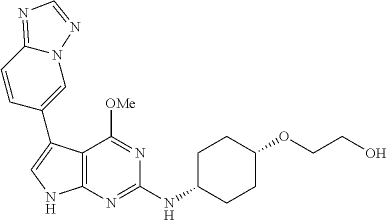 [SMILES: COc1nc(N[C@H]2CC[C@@H](OCCO)CC2)nc2[nH]cc(-c3ccc4ncnn4c3)c12] | 3049 |
| 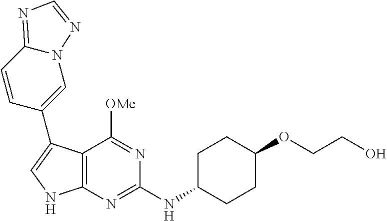 [SMILES: COc1nc(N[C@H]2CC[C@H](OCCO)CC2)nc2[nH]cc(-c3ccc4ncnn4c3)c12] | 3050 |
| 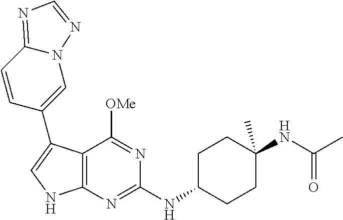 [SMILES: COc1nc(N[C@H]2CC[C@](C)(NC(C)=O)CC2)nc2[nH]cc(-c3ccc4ncnn4c3)c12] | 3051 |
| 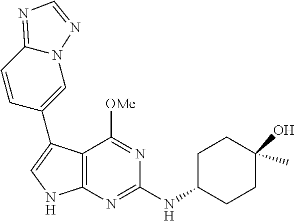 [SMILES: COc1nc(N[C@H]2CC[C@](C)(O)CC2)nc2[nH]cc(-c3ccc4ncnn4c3)c12] | 3052 |
| 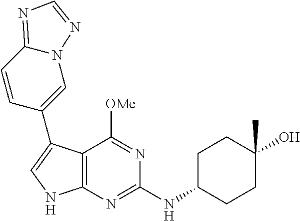 [SMILES: COc1nc(N[C@H]2CC[C@@](C)(O)CC2)nc2[nH]cc(-c3ccc4ncnn4c3)c12] | 3053 |
| 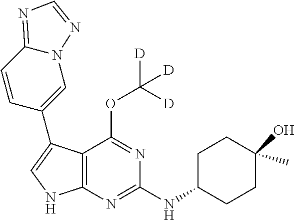 [SMILES: [2H]C([2H])([2H])Oc1nc(N[C@H]2CC[C@](C)(O)CC2)nc2[nH]cc(-c3ccc4ncnn4c3)c12] | 3054 |
| 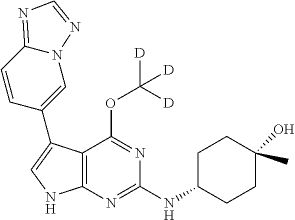 [SMILES: [2H]C([2H])([2H])Oc1nc(N[C@H]2CC[C@@](C)(O)CC2)nc2[nH]cc(-c3ccc4ncnn4c3)c12] | 3055 |
| 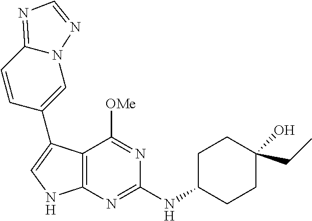 [SMILES: CC[C@]1(O)CC[C@@H](Nc2nc(OC)c3c(-c4ccc5ncnn5c4)c[nH]c3n2)CC1] | 3056 |
| 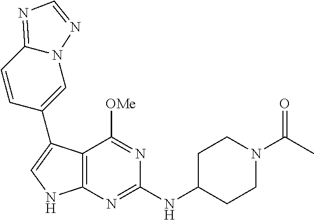 [SMILES: COc1nc(NC2CCN(C(C)=O)CC2)nc2[nH]cc(-c3ccc4ncnn4c3)c12] | 3057 |
| 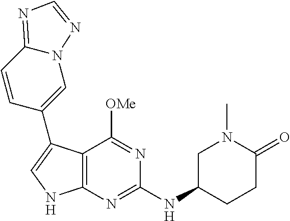 [SMILES: COc1nc(N[C@@H]2CCC(=O)N(C)C2)nc2[nH]cc(-c3ccc4ncnn4c3)c12] | 3058 |
| 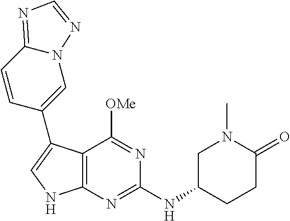 [SMILES: COc1nc(N[C@H]2CCC(=O)N(C)C2)nc2[nH]cc(-c3ccc4ncnn4c3)c12] | 3059 |
| 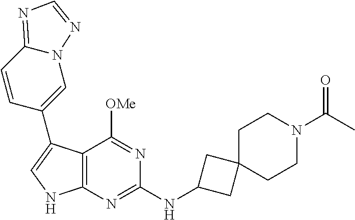 [SMILES: COc1nc(NC2CC3(CCN(C(C)=O)CC3)C2)nc2[nH]cc(-c3ccc4ncnn4c3)c12] | 3060 |
| 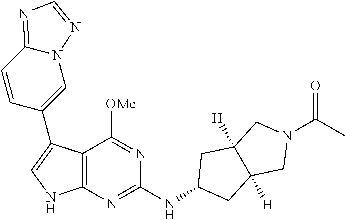 [SMILES: COc1nc(N[C@H]2C[C@@H]3CN(C(C)=O)C[C@@H]3C2)nc2[nH]cc(-c3ccc4ncnn4c3)c12] | 3061 |
| 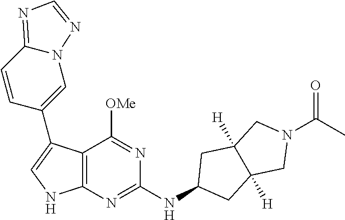 [SMILES: COc1nc(N[C@@H]2C[C@@H]3CN(C(C)=O)C[C@@H]3C2)nc2[nH]cc(-c3ccc4ncnn4c3)c12] | 3062 |
| 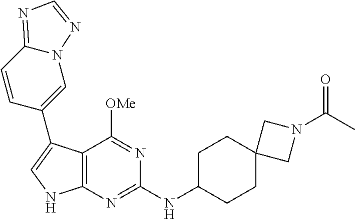 [SMILES: COc1nc(NC2CCC3(CC2)CN(C(C)=O)C3)nc2[nH]cc(-c3ccc4ncnn4c3)c12] | 3063 |
| 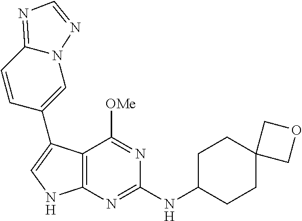 [SMILES: COc1nc(NC2CCC3(CC2)COC3)nc2[nH]cc(-c3ccc4ncnn4c3)c12] | 3064 |
| 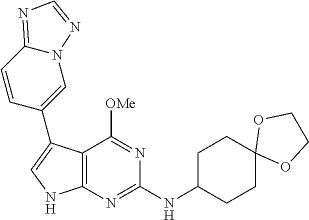 [SMILES: COc1nc(NC2CCC3(CC2)OCCO3)nc2[nH]cc(-c3ccc4ncnn4c3)c12] | 3065 |
| 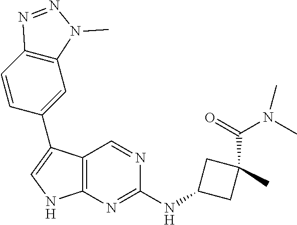 [SMILES: CN(C)C(=O)[C@]1(C)C[C@H](Nc2ncc3c(-c4ccc5nnn(C)c5c4)c[nH]c3n2)C1] | 3066 |
| 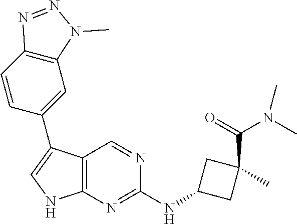 [SMILES: CN(C)C(=O)[C@]1(C)C[C@@H](Nc2ncc3c(-c4ccc5nnn(C)c5c4)c[nH]c3n2)C1] | 3067 |
| 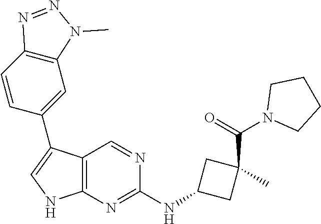 [SMILES: Cn1nnc2ccc(-c3c[nH]c4nc(N[C@H]5C[C@](C)(C(=O)N6CCCC6)C5)ncc34)cc21] | 3068 |
| 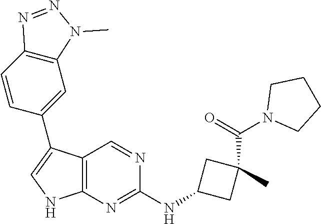 [SMILES: Cn1nnc2ccc(-c3c[nH]c4nc(N[C@H]5C[C@@](C)(C(=O)N6CCCC6)C5)ncc34)cc21] | 3069 |
| 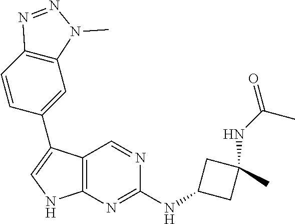 [SMILES: CC(=O)N[C@]1(C)C[C@H](Nc2ncc3c(-c4ccc5nnn(C)c5c4)c[nH]c3n2)C1] | 3070 |
| 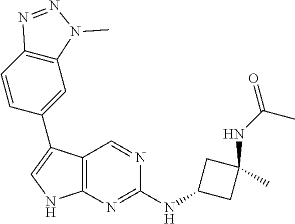 [SMILES: CC(=O)N[C@]1(C)C[C@@H](Nc2ncc3c(-c4ccc5nnn(C)c5c4)c[nH]c3n2)C1] | 3071 |
| 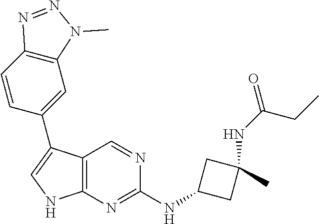 [SMILES: CCC(=O)N[C@]1(C)C[C@H](Nc2ncc3c(-c4ccc5nnn(C)c5c4)c[nH]c3n2)C1] | 3072 |
| 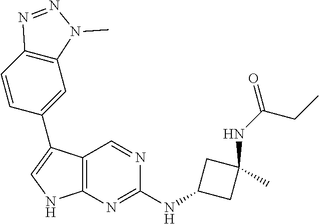 [SMILES: CCC(=O)N[C@]1(C)C[C@@H](Nc2ncc3c(-c4ccc5nnn(C)c5c4)c[nH]c3n2)C1] | 3073 |
| 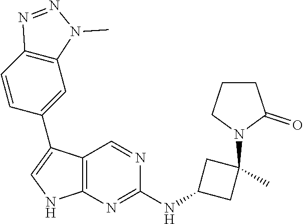 [SMILES: Cn1nnc2ccc(-c3c[nH]c4nc(N[C@H]5C[C@](C)(N6CCCC6=O)C5)ncc34)cc21] | 3074 |
| 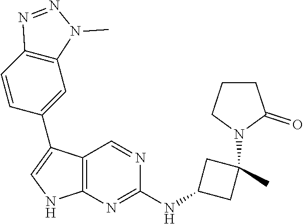 [SMILES: Cn1nnc2ccc(-c3c[nH]c4nc(N[C@H]5C[C@@](C)(N6CCCC6=O)C5)ncc34)cc21] | 3075 |
| 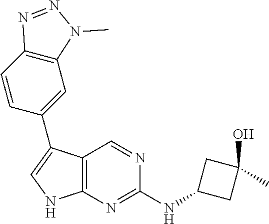 [SMILES: Cn1nnc2ccc(-c3c[nH]c4nc(N[C@H]5C[C@](C)(O)C5)ncc34)cc21] | 3076 |
| 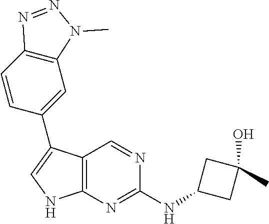 [SMILES: Cn1nnc2ccc(-c3c[nH]c4nc(N[C@H]5C[C@@](C)(O)C5)ncc34)cc21] | 3077 |
| 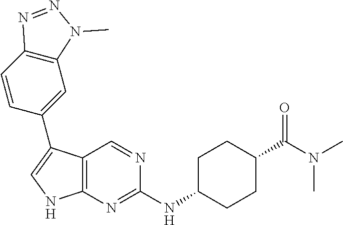 [SMILES: CN(C)C(=O)[C@H]1CC[C@@H](Nc2ncc3c(-c4ccc5nnn(C)c5c4)c[nH]c3n2)CC1] | 3078 |
| 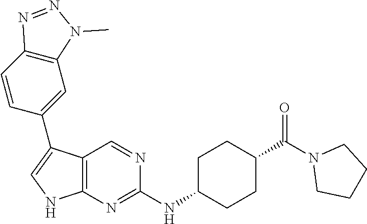 [SMILES: Cn1nnc2ccc(-c3c[nH]c4nc(N[C@H]5CC[C@@H](C(=O)N6CCCC6)CC5)ncc34)cc21] | 3079 |
| 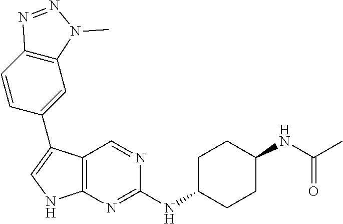 [SMILES: CC(=O)N[C@H]1CC[C@H](Nc2ncc3c(-c4ccc5nnn(C)c5c4)c[nH]c3n2)CC1] | 3080 |
| 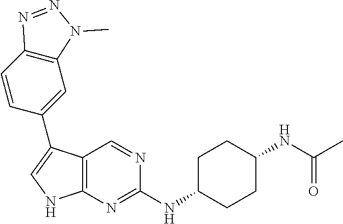 [SMILES: CC(=O)N[C@H]1CC[C@@H](Nc2ncc3c(-c4ccc5nnn(C)c5c4)c[nH]c3n2)CC1] | 3081 |
| 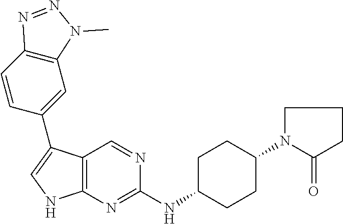 [SMILES: Cn1nnc2ccc(-c3c[nH]c4nc(N[C@H]5CC[C@@H](N6CCCC6=O)CC5)ncc34)cc21] | 3082 |
| 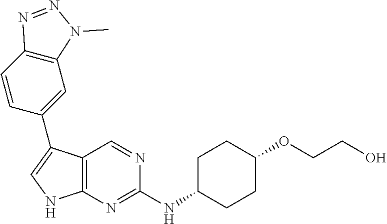 [SMILES: Cn1nnc2ccc(-c3c[nH]c4nc(N[C@H]5CC[C@@H](OCCO)CC5)ncc34)cc21] | 3083 |
| 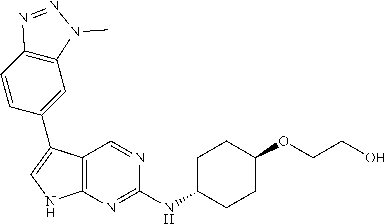 [SMILES: Cn1nnc2ccc(-c3c[nH]c4nc(N[C@H]5CC[C@H](OCCO)CC5)ncc34)cc21] | 3084 |
| 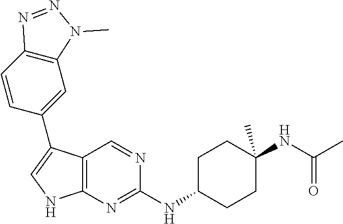 [SMILES: CC(=O)N[C@]1(C)CC[C@@H](Nc2ncc3c(-c4ccc5nnn(C)c5c4)c[nH]c3n2)CC1] | 3085 |
| 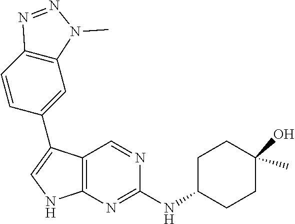 [SMILES: Cn1nnc2ccc(-c3c[nH]c4nc(N[C@H]5CC[C@](C)(O)CC5)ncc34)cc21] | 3086 |
| 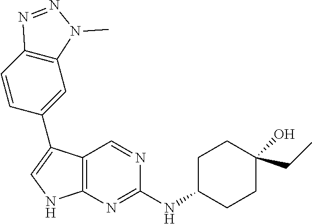 [SMILES: CC[C@]1(O)CC[C@@H](Nc2ncc3c(-c4ccc5nnn(C)c5c4)c[nH]c3n2)CC1] | 3087 |
| 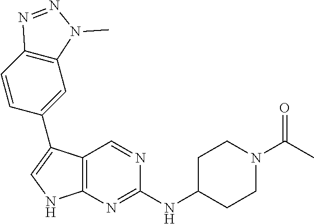 [SMILES: CC(=O)N1CCC(Nc2ncc3c(-c4ccc5nnn(C)c5c4)c[nH]c3n2)CC1] | 3088 |
| 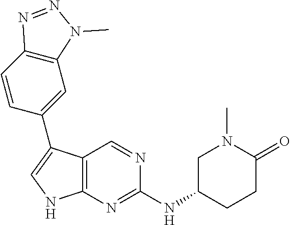 [SMILES: CN1C[C@@H](Nc2ncc3c(-c4ccc5nnn(C)c5c4)c[nH]c3n2)CCC1=O] | 3089 |
| 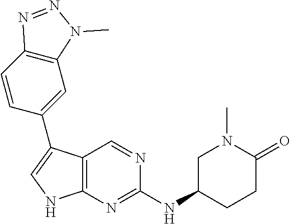 [SMILES: CN1C[C@H](Nc2ncc3c(-c4ccc5nnn(C)c5c4)c[nH]c3n2)CCC1=O] | 3090 |
| 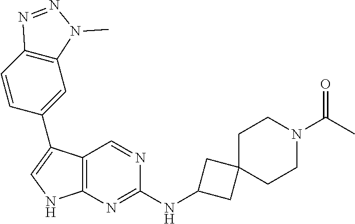 [SMILES: CC(=O)N1CCC2(CC1)CC(Nc1ncc3c(-c4ccc5nnn(C)c5c4)c[nH]c3n1)C2] | 3091 |
| 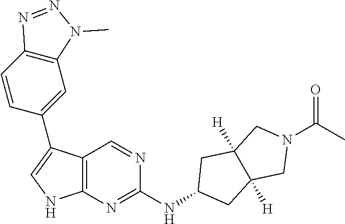 [SMILES: CC(=O)N1C[C@H]2C[C@H](Nc3ncc4c(-c5ccc6nnn(C)c6c5)c[nH]c4n3)C[C@H]2C1] | 3092 |
| 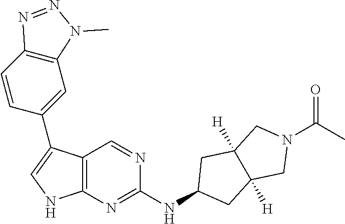 [SMILES: CC(=O)N1C[C@H]2C[C@@H](Nc3ncc4c(-c5ccc6nnn(C)c6c5)c[nH]c4n3)C[C@H]2C1] | 3093 |
| 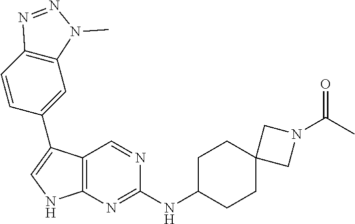 [SMILES: CC(=O)N1CC2(CCC(Nc3ncc4c(-c5ccc6nnn(C)c6c5)c[nH]c4n3)CC2)C1] | 3094 |
| 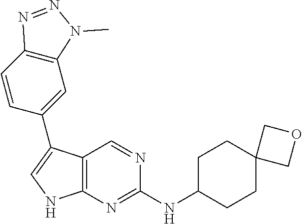 [SMILES: Cn1nnc2ccc(-c3c[nH]c4nc(NC5CCC6(CC5)COC6)ncc34)cc21] | 3095 |
| 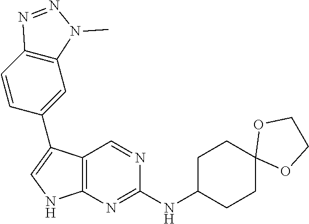 [SMILES: Cn1nnc2ccc(-c3c[nH]c4nc(NC5CCC6(CC5)OCCO6)ncc34)cc21] | 3096 |
| 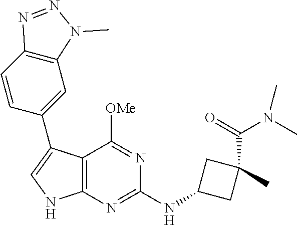 [SMILES: COc1nc(N[C@H]2C[C@@](C)(C(=O)N(C)C)C2)nc2[nH]cc(-c3ccc4nnn(C)c4c3)c12] | 3097 |
| 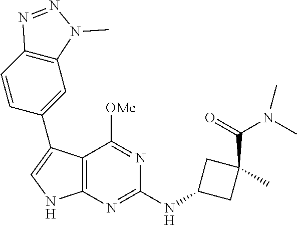 [SMILES: COc1nc(N[C@H]2C[C@](C)(C(=O)N(C)C)C2)nc2[nH]cc(-c3ccc4nnn(C)c4c3)c12] | 3098 |
| 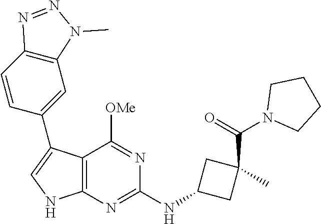 [SMILES: COc1nc(N[C@H]2C[C@](C)(C(=O)N3CCCC3)C2)nc2[nH]cc(-c3ccc4nnn(C)c4c3)c12] | 3099 |
| 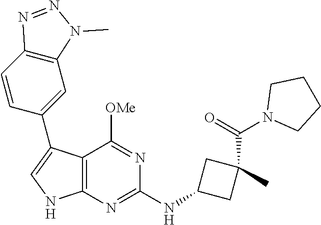 [SMILES: COc1nc(N[C@H]2C[C@@](C)(C(=O)N3CCCC3)C2)nc2[nH]cc(-c3ccc4nnn(C)c4c3)c12] | 3100 |
| 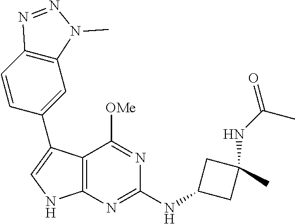 [SMILES: COc1nc(N[C@H]2C[C@@](C)(NC(C)=O)C2)nc2[nH]cc(-c3ccc4nnn(C)c4c3)c12] | 3101 |
| 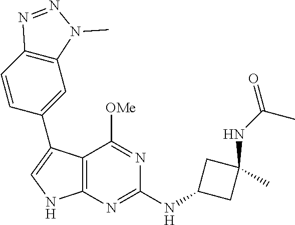 [SMILES: COc1nc(N[C@H]2C[C@](C)(NC(C)=O)C2)nc2[nH]cc(-c3ccc4nnn(C)c4c3)c12] | 3102 |
| 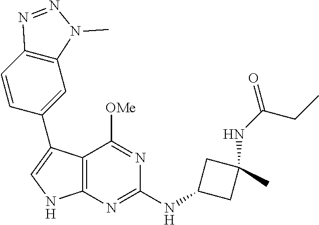 [SMILES: CCC(=O)N[C@]1(C)C[C@H](Nc2nc(OC)c3c(-c4ccc5nnn(C)c5c4)c[nH]c3n2)C1] | 3103 |
| 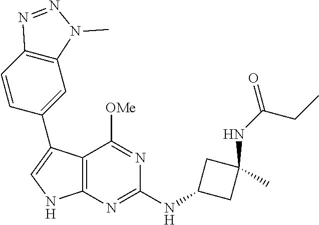 [SMILES: CCC(=O)N[C@]1(C)C[C@@H](Nc2nc(OC)c3c(-c4ccc5nnn(C)c5c4)c[nH]c3n2)C1] | 3104 |
| 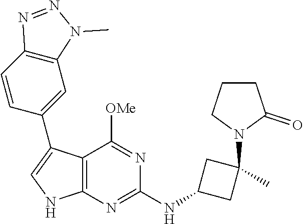 [SMILES: COc1nc(N[C@H]2C[C@](C)(N3CCCC3=O)C2)nc2[nH]cc(-c3ccc4nnn(C)c4c3)c12] | 3105 |
| 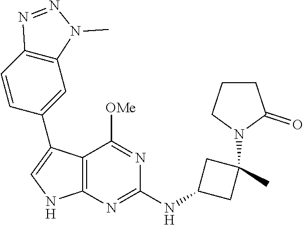 [SMILES: COc1nc(N[C@H]2C[C@@](C)(N3CCCC3=O)C2)nc2[nH]cc(-c3ccc4nnn(C)c4c3)c12] | 3106 |
| 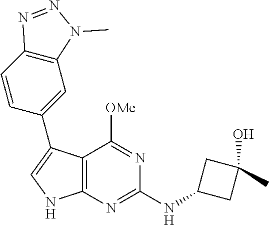 [SMILES: COc1nc(N[C@H]2C[C@@](C)(O)C2)nc2[nH]cc(-c3ccc4nnn(C)c4c3)c12] | 3107 |
| 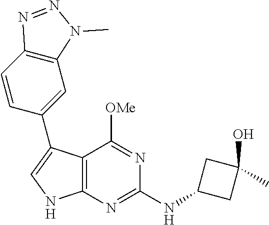 [SMILES: COc1nc(N[C@H]2C[C@](C)(O)C2)nc2[nH]cc(-c3ccc4nnn(C)c4c3)c12] | 3108 |
| 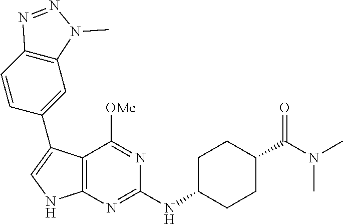 [SMILES: COc1nc(N[C@H]2CC[C@@H](C(=O)N(C)C)CC2)nc2[nH]cc(-c3ccc4nnn(C)c4c3)c12] | 3109 |
| 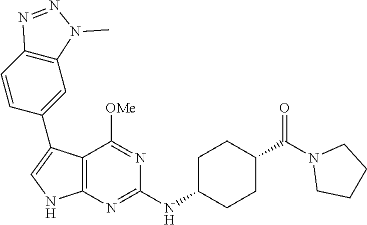 [SMILES: COc1nc(N[C@H]2CC[C@@H](C(=O)N3CCCC3)CC2)nc2[nH]cc(-c3ccc4nnn(C)c4c3)c12] | 3110 |
| 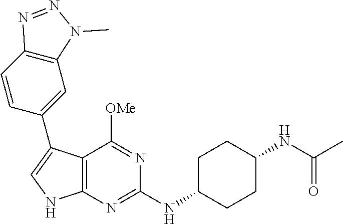 [SMILES: COc1nc(N[C@H]2CC[C@@H](NC(C)=O)CC2)nc2[nH]cc(-c3ccc4nnn(C)c4c3)c12] | 3111 |
| 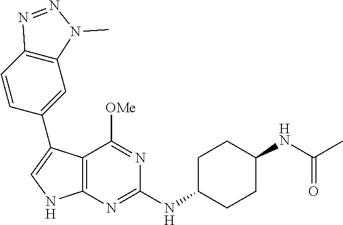 [SMILES: COc1nc(N[C@H]2CC[C@H](NC(C)=O)CC2)nc2[nH]cc(-c3ccc4nnn(C)c4c3)c12] | 3112 |
| 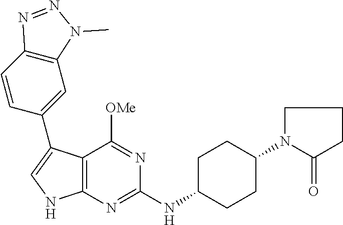 [SMILES: COc1nc(N[C@H]2CC[C@@H](N3CCCC3=O)CC2)nc2[nH]cc(-c3ccc4nnn(C)c4c3)c12] | 3113 |
| 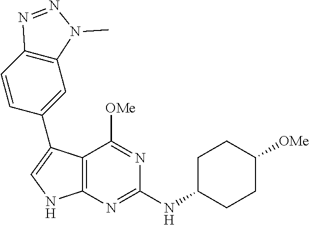 [SMILES: COc1nc(N[C@H]2CC[C@@H](OC)CC2)nc2[nH]cc(-c3ccc4nnn(C)c4c3)c12] | 3114 |
| 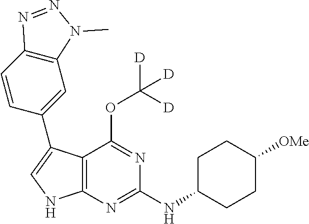 [SMILES: [2H]C([2H])([2H])Oc1nc(N[C@H]2CC[C@@H](OC)CC2)nc2[nH]cc(-c3ccc4nnn(C)c4c3)c12] | 3115 |
| 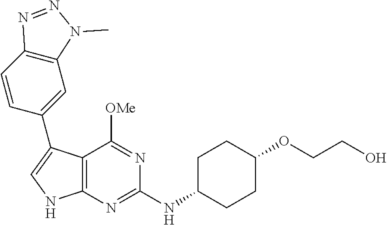 [SMILES: COc1nc(N[C@H]2CC[C@@H](OCCO)CC2)nc2[nH]cc(-c3ccc4nnn(C)c4c3)c12] | 3116 |
| 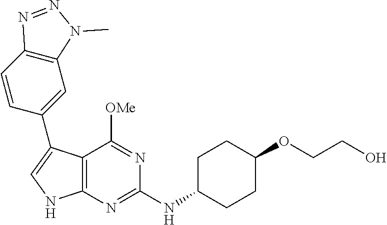 [SMILES: COc1nc(N[C@H]2CC[C@H](OCCO)CC2)nc2[nH]cc(-c3ccc4nnn(C)c4c3)c12] | 3117 |
| 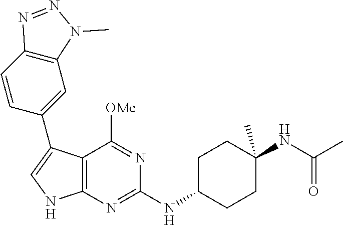 [SMILES: COc1nc(N[C@H]2CC[C@](C)(NC(C)=O)CC2)nc2[nH]cc(-c3ccc4nnn(C)c4c3)c12] | 3118 |
| 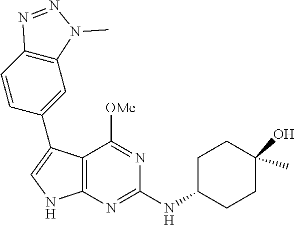 [SMILES: COc1nc(N[C@H]2CC[C@](C)(O)CC2)nc2[nH]cc(-c3ccc4nnn(C)c4c3)c12] | 3119 |
| 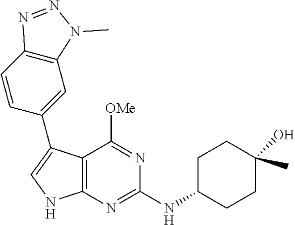 [SMILES: COc1nc(N[C@H]2CC[C@@](C)(O)CC2)nc2[nH]cc(-c3ccc4nnn(C)c4c3)c12] | 3120 |
| 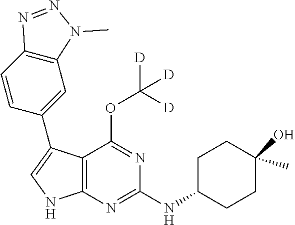 [SMILES: [2H]C([2H])([2H])Oc1nc(N[C@H]2CC[C@](C)(O)CC2)nc2[nH]cc(-c3ccc4nnn(C)c4c3)c12] | 3121 |
| 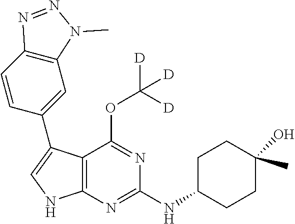 [SMILES: [2H]C([2H])([2H])Oc1nc(N[C@H]2CC[C@@](C)(O)CC2)nc2[nH]cc(-c3ccc4nnn(C)c4c3)c12] | 3122 |
| 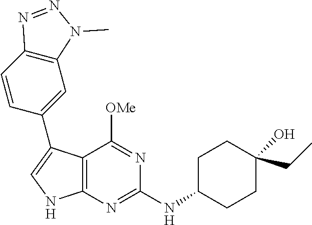 [SMILES: CC[C@]1(O)CC[C@@H](Nc2nc(OC)c3c(-c4ccc5nnn(C)c5c4)c[nH]c3n2)CC1] | 3123 |
| 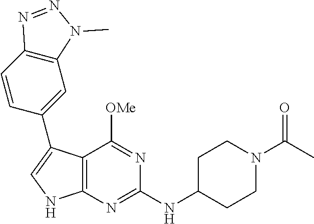 [SMILES: COc1nc(NC2CCN(C(C)=O)CC2)nc2[nH]cc(-c3ccc4nnn(C)c4c3)c12] | 3124 |
| 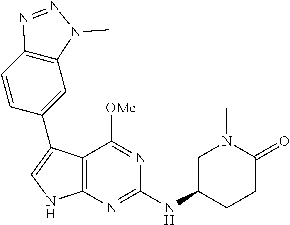 [SMILES: COc1nc(N[C@@H]2CCC(=O)N(C)C2)nc2[nH]cc(-c3ccc4nnn(C)c4c3)c12] | 3125 |
| 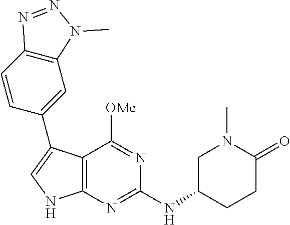 [SMILES: COc1nc(N[C@H]2CCC(=O)N(C)C2)nc2[nH]cc(-c3ccc4nnn(C)c4c3)c12] | 3126 |
| 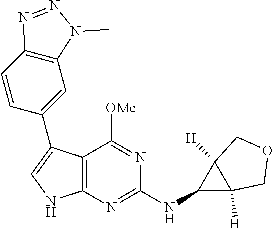 [SMILES: COc1nc(N[C@@H]2[C@@H]3COC[C@@H]32)nc2[nH]cc(-c3ccc4nnn(C)c4c3)c12] | 3127 |
| 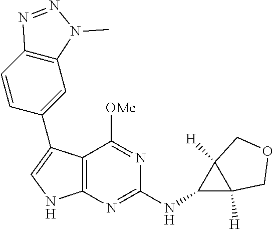 [SMILES: COc1nc(N[C@H]2[C@@H]3COC[C@@H]32)nc2[nH]cc(-c3ccc4nnn(C)c4c3)c12] | 3128 |
| 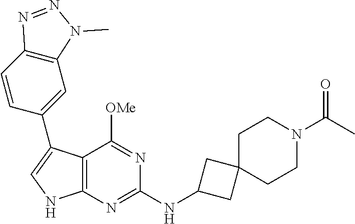 [SMILES: COc1nc(NC2CC3(CCN(C(C)=O)CC3)C2)nc2[nH]cc(-c3ccc4nnn(C)c4c3)c12] | 3129 |
| 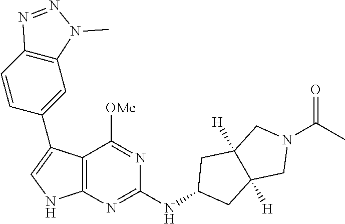 [SMILES: COc1nc(N[C@H]2C[C@@H]3CN(C(C)=O)C[C@@H]3C2)nc2[nH]cc(-c3ccc4nnn(C)c4c3)c12] | 3130 |
| 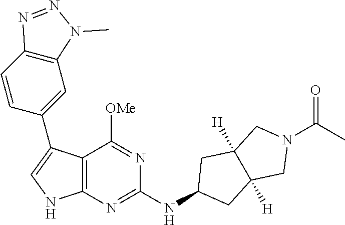 [SMILES: COc1nc(N[C@@H]2C[C@@H]3CN(C(C)=O)C[C@@H]3C2)nc2[nH]cc(-c3ccc4nnn(C)c4c3)c12] | 3131 |
| 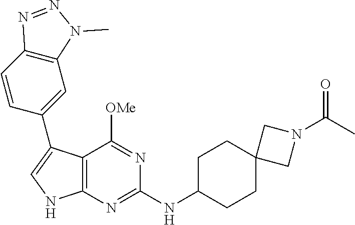 [SMILES: COc1nc(NC2CCC3(CC2)CN(C(C)=O)C3)nc2[nH]cc(-c3ccc4nnn(C)c4c3)c12] | 3132 |
| 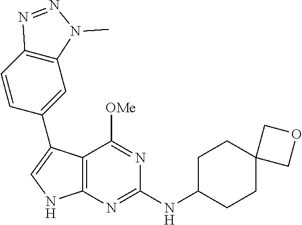 [SMILES: COc1nc(NC2CCC3(CC2)COC3)nc2[nH]cc(-c3ccc4nnn(C)c4c3)c12] | 3133 |
| 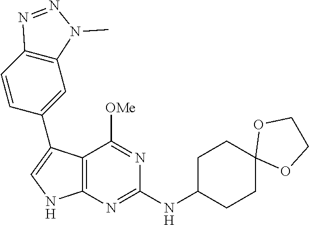 [SMILES: COc1nc(NC2CCC3(CC2)OCCO3)nc2[nH]cc(-c3ccc4nnn(C)c4c3)c12] | 3134 |
| 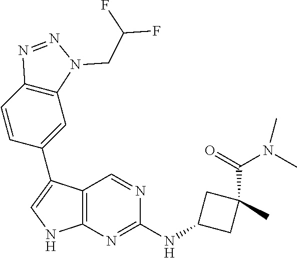 [SMILES: CN(C)C(=O)[C@]1(C)C[C@H](Nc2ncc3c(-c4ccc5nnn(CC(F)F)c5c4)c[nH]c3n2)C1] | 3135 |
| 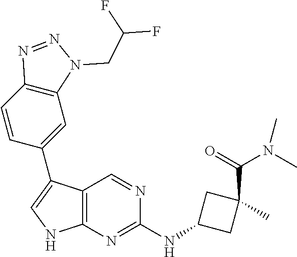 [SMILES: CN(C)C(=O)[C@]1(C)C[C@@H](Nc2ncc3c(-c4ccc5nnn(CC(F)F)c5c4)c[nH]c3n2)C1] | 3136 |
| 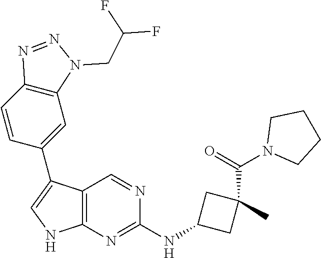 [SMILES: C[C@]1(C(=O)N2CCCC2)C[C@@H](Nc2ncc3c(-c4ccc5nnn(CC(F)F)c5c4)c[nH]c3n2)C1] | 3137 |
| 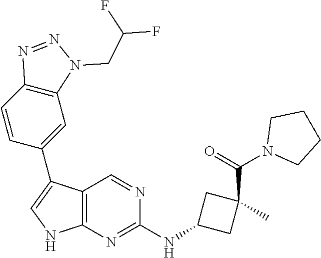 [SMILES: C[C@]1(C(=O)N2CCCC2)C[C@H](Nc2ncc3c(-c4ccc5nnn(CC(F)F)c5c4)c[nH]c3n2)C1] | 3138 |
| 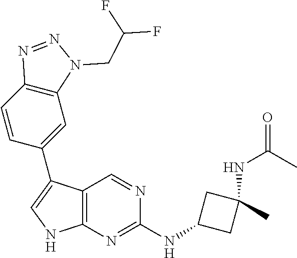 [SMILES: CC(=O)N[C@]1(C)C[C@H](Nc2ncc3c(-c4ccc5nnn(CC(F)F)c5c4)c[nH]c3n2)C1] | 3139 |
| 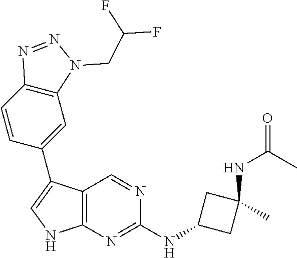 [SMILES: CC(=O)N[C@]1(C)C[C@@H](Nc2ncc3c(-c4ccc5nnn(CC(F)F)c5c4)c[nH]c3n2)C1] | 3140 |
| 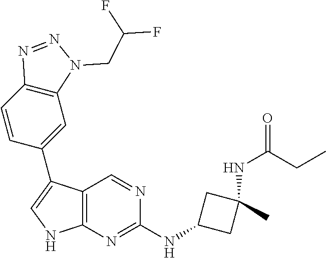 [SMILES: CCC(=O)N[C@]1(C)C[C@H](Nc2ncc3c(-c4ccc5nnn(CC(F)F)c5c4)c[nH]c3n2)C1] | 3141 |
| 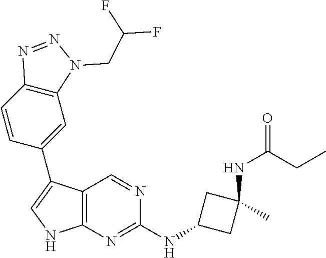 [SMILES: CCC(=O)N[C@]1(C)C[C@@H](Nc2ncc3c(-c4ccc5nnn(CC(F)F)c5c4)c[nH]c3n2)C1] | 3142 |
| 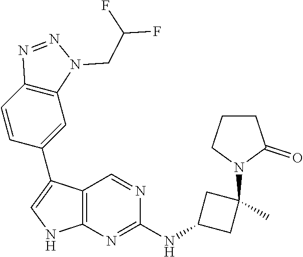 [SMILES: C[C@]1(N2CCCC2=O)C[C@H](Nc2ncc3c(-c4ccc5nnn(CC(F)F)c5c4)c[nH]c3n2)C1] | 3143 |
| 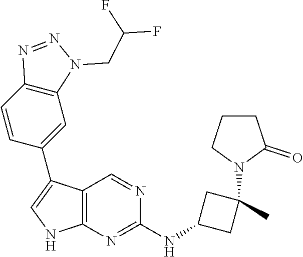 [SMILES: C[C@]1(N2CCCC2=O)C[C@@H](Nc2ncc3c(-c4ccc5nnn(CC(F)F)c5c4)c[nH]c3n2)C1] | 3144 |
| 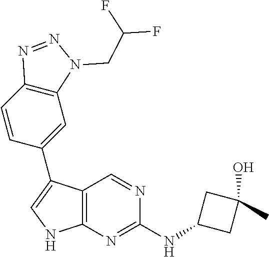 [SMILES: C[C@]1(O)C[C@@H](Nc2ncc3c(-c4ccc5nnn(CC(F)F)c5c4)c[nH]c3n2)C1] | 3145 |
| 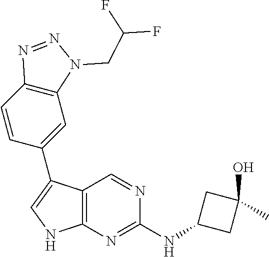 [SMILES: C[C@]1(O)C[C@H](Nc2ncc3c(-c4ccc5nnn(CC(F)F)c5c4)c[nH]c3n2)C1] | 3146 |
| 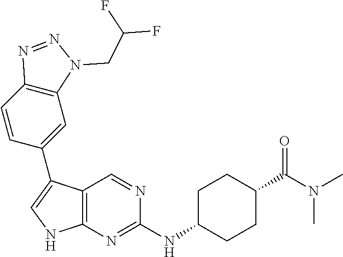 [SMILES: CN(C)C(=O)[C@H]1CC[C@@H](Nc2ncc3c(-c4ccc5nnn(CC(F)F)c5c4)c[nH]c3n2)CC1] | 3147 |
| 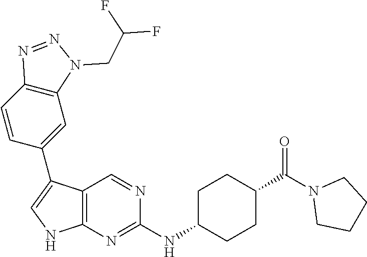 [SMILES: O=C([C@H]1CC[C@@H](Nc2ncc3c(-c4ccc5nnn(CC(F)F)c5c4)c[nH]c3n2)CC1)N1CCCC1] | 3148 |
| 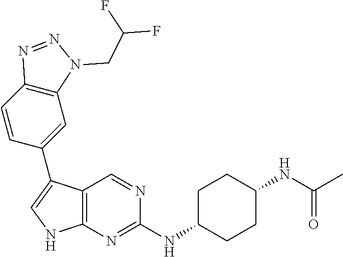 [SMILES: CC(=O)N[C@H]1CC[C@@H](Nc2ncc3c(-c4ccc5nnn(CC(F)F)c5c4)c[nH]c3n2)CC1] | 3149 |
| 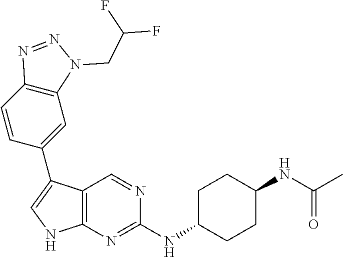 [SMILES: CC(=O)N[C@H]1CC[C@H](Nc2ncc3c(-c4ccc5nnn(CC(F)F)c5c4)c[nH]c3n2)CC1] | 3150 |
| 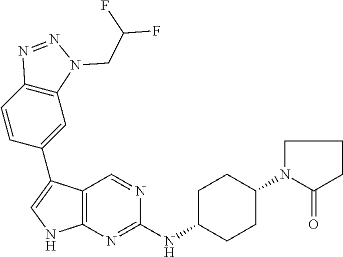 [SMILES: O=C1CCCN1[C@H]1CC[C@@H](Nc2ncc3c(-c4ccc5nnn(CC(F)F)c5c4)c[nH]c3n2)CC1] | 3151 |
| 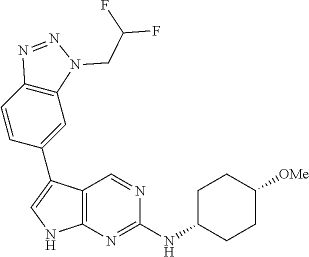 [SMILES: CO[C@H]1CC[C@@H](Nc2ncc3c(-c4ccc5nnn(CC(F)F)c5c4)c[nH]c3n2)CC1] | 3152 |
| 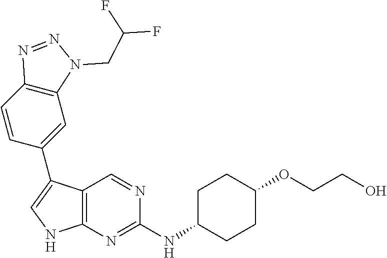 [SMILES: OCCO[C@H]1CC[C@@H](Nc2ncc3c(-c4ccc5nnn(CC(F)F)c5c4)c[nH]c3n2)CC1] | 3153 |
| 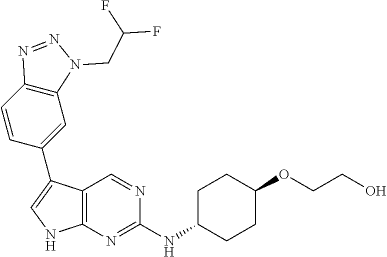 [SMILES: OCCO[C@H]1CC[C@H](Nc2ncc3c(-c4ccc5nnn(CC(F)F)c5c4)c[nH]c3n2)CC1] | 3154 |
| 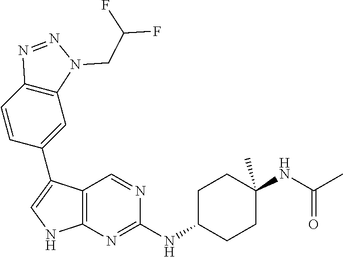 [SMILES: CC(=O)N[C@]1(C)CC[C@@H](Nc2ncc3c(-c4ccc5nnn(CC(F)F)c5c4)c[nH]c3n2)CC1] | 3155 |
| 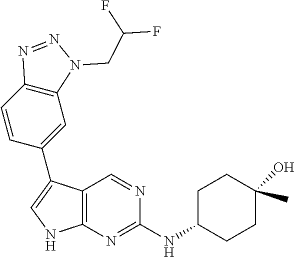 [SMILES: C[C@]1(O)CC[C@@H](Nc2ncc3c(-c4ccc5nnn(CC(F)F)c5c4)c[nH]c3n2)CC1] | 3156 |
| 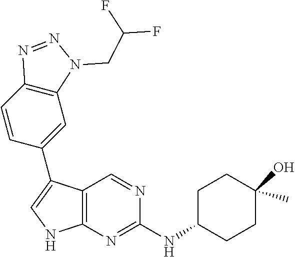 [SMILES: C[C@]1(O)CC[C@H](Nc2ncc3c(-c4ccc5nnn(CC(F)F)c5c4)c[nH]c3n2)CC1] | 3157 |
| 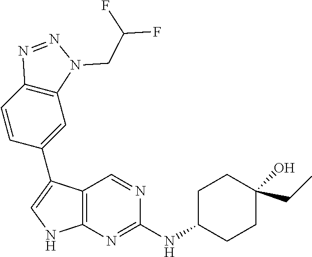 [SMILES: CC[C@]1(O)CC[C@@H](Nc2ncc3c(-c4ccc5nnn(CC(F)F)c5c4)c[nH]c3n2)CC1] | 3158 |
| 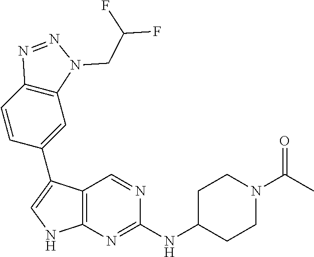 [SMILES: CC(=O)N1CCC(Nc2ncc3c(-c4ccc5nnn(CC(F)F)c5c4)c[nH]c3n2)CC1] | 3159 |
| 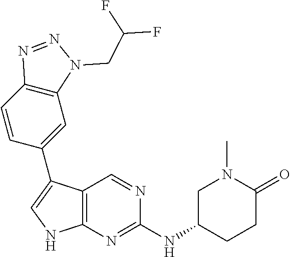 [SMILES: CN1C[C@@H](Nc2ncc3c(-c4ccc5nnn(CC(F)F)c5c4)c[nH]c3n2)CCC1=O] | 3160 |
| 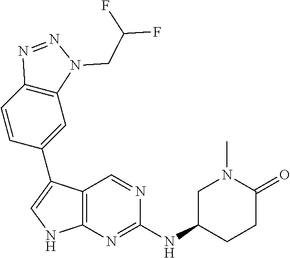 [SMILES: CN1C[C@H](Nc2ncc3c(-c4ccc5nnn(CC(F)F)c5c4)c[nH]c3n2)CCC1=O] | 3161 |
| 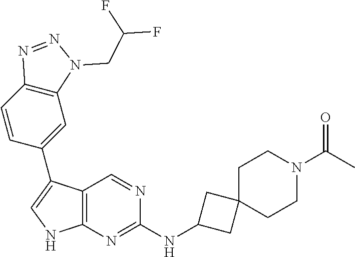 [SMILES: CC(=O)N1CCC2(CC1)CC(Nc1ncc3c(-c4ccc5nnn(CC(F)F)c5c4)c[nH]c3n1)C2] | 3162 |
| 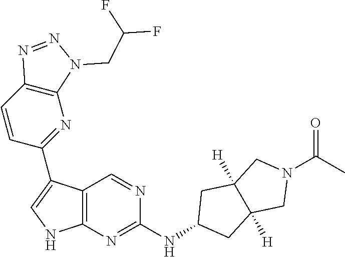 [SMILES: CC(=O)N1C[C@H]2C[C@H](Nc3ncc4c(-c5ccc6nnn(CC(F)F)c6n5)c[nH]c4n3)C[C@H]2C1] | 3163 |
| 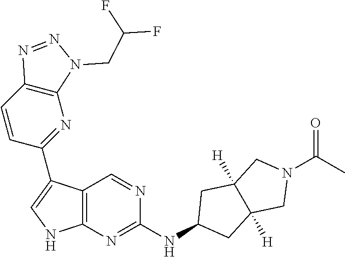 [SMILES: CC(=O)N1C[C@H]2C[C@@H](Nc3ncc4c(-c5ccc6nnn(CC(F)F)c6n5)c[nH]c4n3)C[C@H]2C1] | 3164 |
| 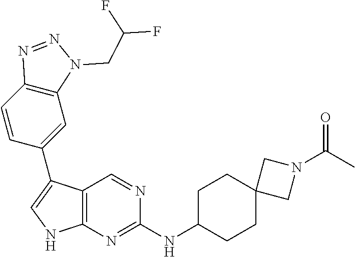 [SMILES: CC(=O)N1CC2(CCC(Nc3ncc4c(-c5ccc6nnn(CC(F)F)c6c5)c[nH]c4n3)CC2)C1] | 3165 |
| 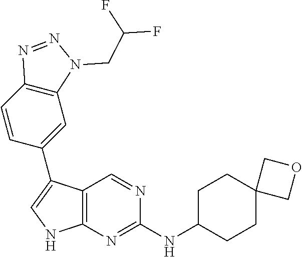 [SMILES: FC(F)Cn1nnc2ccc(-c3c[nH]c4nc(NC5CCC6(CC5)COC6)ncc34)cc21] | 3166 |
| 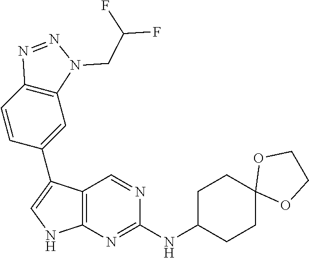 [SMILES: FC(F)Cn1nnc2ccc(-c3c[nH]c4nc(NC5CCC6(CC5)OCCO6)ncc34)cc21] | 3167 |
| 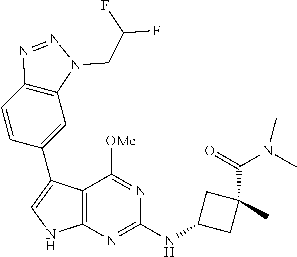 [SMILES: COc1nc(N[C@H]2C[C@@](C)(C(=O)N(C)C)C2)nc2[nH]cc(-c3ccc4nnn(CC(F)F)c4c3)c12] | 3168 |
| 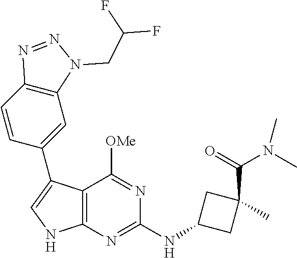 [SMILES: COc1nc(N[C@H]2C[C@](C)(C(=O)N(C)C)C2)nc2[nH]cc(-c3ccc4nnn(CC(F)F)c4c3)c12] | 3169 |
| 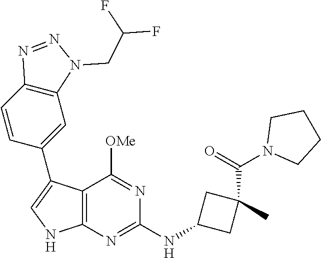 [SMILES: COc1nc(N[C@H]2C[C@@](C)(C(=O)N3CCCC3)C2)nc2[nH]cc(-c3ccc4nnn(CC(F)F)c4c3)c12] | 3170 |
| 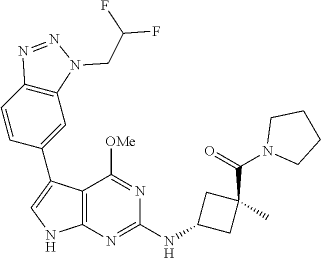 [SMILES: COc1nc(N[C@H]2C[C@](C)(C(=O)N3CCCC3)C2)nc2[nH]cc(-c3ccc4nnn(CC(F)F)c4c3)c12] | 3171 |
| 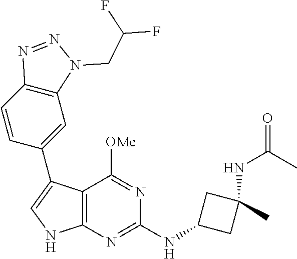 [SMILES: COc1nc(N[C@H]2C[C@@](C)(NC(C)=O)C2)nc2[nH]cc(-c3ccc4nnn(CC(F)F)c4c3)c12] | 3172 |
| 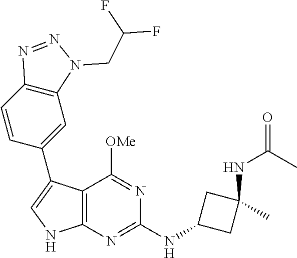 [SMILES: COc1nc(N[C@H]2C[C@](C)(NC(C)=O)C2)nc2[nH]cc(-c3ccc4nnn(CC(F)F)c4c3)c12] | 3173 |
| 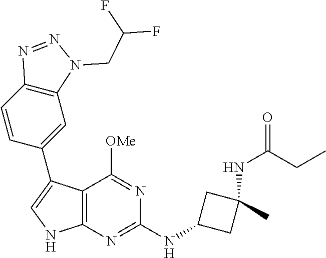 [SMILES: CCC(=O)N[C@]1(C)C[C@H](Nc2nc(OC)c3c(-c4ccc5nnn(CC(F)F)c5c4)c[nH]c3n2)C1] | 3174 |
| 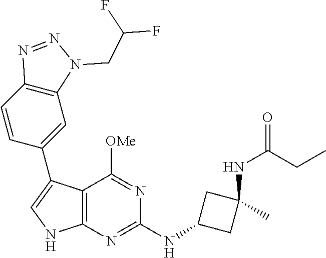 [SMILES: CCC(=O)N[C@]1(C)C[C@@H](Nc2nc(OC)c3c(-c4ccc5nnn(CC(F)F)c5c4)c[nH]c3n2)C1] | 3175 |
| 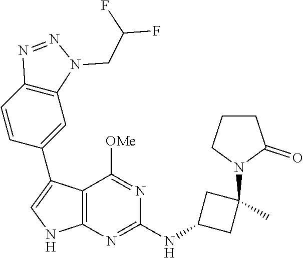 [SMILES: COc1nc(N[C@H]2C[C@](C)(N3CCCC3=O)C2)nc2[nH]cc(-c3ccc4nnn(CC(F)F)c4c3)c12] | 3176 |
| 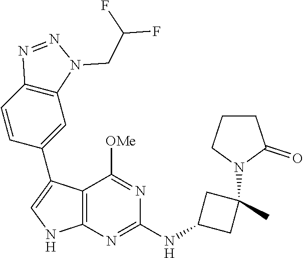 [SMILES: COc1nc(N[C@H]2C[C@@](C)(N3CCCC3=O)C2)nc2[nH]cc(-c3ccc4nnn(CC(F)F)c4c3)c12] | 3177 |
| 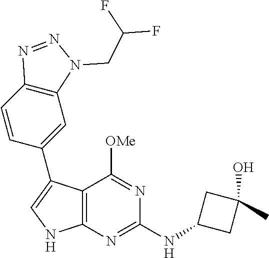 [SMILES: COc1nc(N[C@H]2C[C@@](C)(O)C2)nc2[nH]cc(-c3ccc4nnn(CC(F)F)c4c3)c12] | 3178 |
| 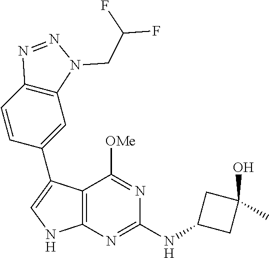 [SMILES: COc1nc(N[C@H]2C[C@](C)(O)C2)nc2[nH]cc(-c3ccc4nnn(CC(F)F)c4c3)c12] | 3179 |
| 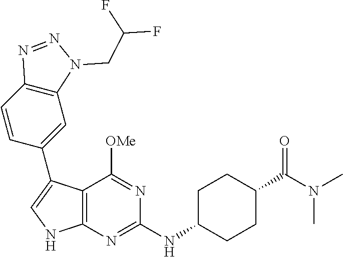 [SMILES: COc1nc(N[C@H]2CC[C@@H](C(=O)N(C)C)CC2)nc2[nH]cc(-c3ccc4nnn(CC(F)F)c4c3)c12] | 3180 |
| 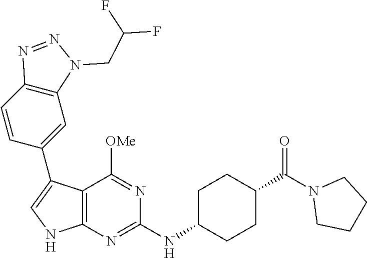 [SMILES: COc1nc(N[C@H]2CC[C@@H](C(=O)N3CCCC3)CC2)nc2[nH]cc(-c3ccc4nnn(CC(F)F)c4c3)c12] | 3181 |
| 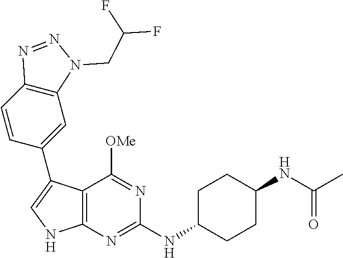 [SMILES: COc1nc(N[C@H]2CC[C@H](NC(C)=O)CC2)nc2[nH]cc(-c3ccc4nnn(CC(F)F)c4c3)c12] | 3182 |
| 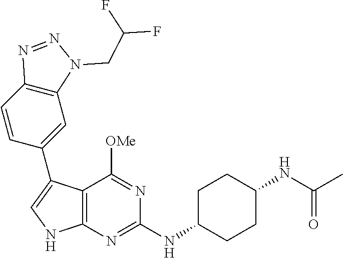 [SMILES: COc1nc(N[C@H]2CC[C@@H](NC(C)=O)CC2)nc2[nH]cc(-c3ccc4nnn(CC(F)F)c4c3)c12] | 3183 |
| 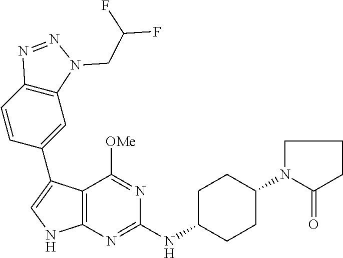 [SMILES: COc1nc(N[C@H]2CC[C@@H](N3CCCC3=O)CC2)nc2[nH]cc(-c3ccc4nnn(CC(F)F)c4c3)c12] | 3184 |
| 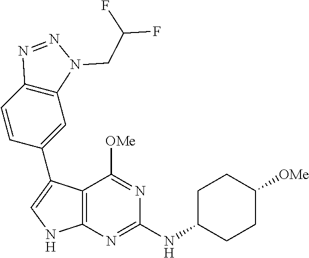 [SMILES: COc1nc(N[C@H]2CC[C@@H](OC)CC2)nc2[nH]cc(-c3ccc4nnn(CC(F)F)c4c3)c12] | 3185 |
| 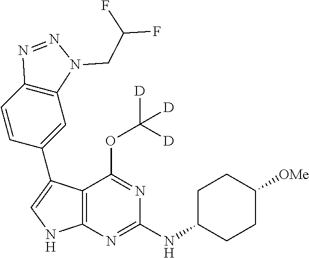 [SMILES: [2H]C([2H])([2H])Oc1nc(N[C@H]2CC[C@@H](OC)CC2)nc2[nH]cc(-c3ccc4nnn(CC(F)F)c4c3)c12] | 3186 |
| 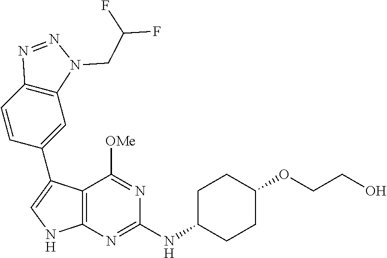 [SMILES: COc1nc(N[C@H]2CC[C@@H](OCCO)CC2)nc2[nH]cc(-c3ccc4nnn(CC(F)F)c4c3)c12] | 3187 |
| 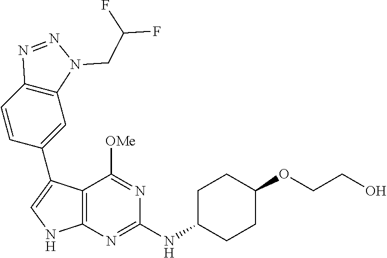 [SMILES: COc1nc(N[C@H]2CC[C@H](OCCO)CC2)nc2[nH]cc(-c3ccc4nnn(CC(F)F)c4c3)c12] | 3188 |
| 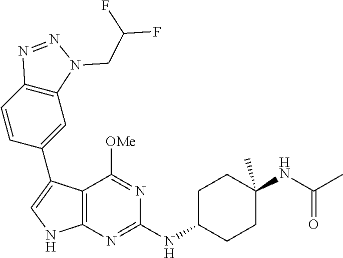 [SMILES: COc1nc(N[C@H]2CC[C@](C)(NC(C)=O)CC2)nc2[nH]cc(-c3ccc4nnn(CC(F)F)c4c3)c12] | 3189 |
| 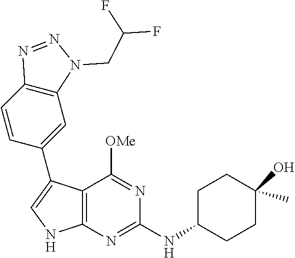 [SMILES: COc1nc(N[C@H]2CC[C@](C)(O)CC2)nc2[nH]cc(-c3ccc4nnn(CC(F)F)c4c3)c12] | 3190 |
| 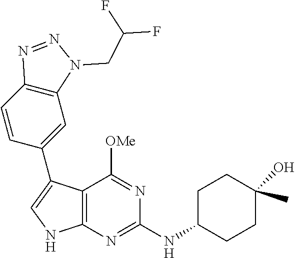 [SMILES: COc1nc(N[C@H]2CC[C@@](C)(O)CC2)nc2[nH]cc(-c3ccc4nnn(CC(F)F)c4c3)c12] | 3191 |
| 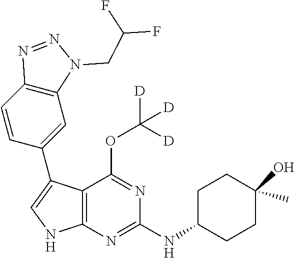 [SMILES: [2H]C([2H])([2H])Oc1nc(N[C@H]2CC[C@](C)(O)CC2)nc2[nH]cc(-c3ccc4nnn(CC(F)F)c4c3)c12] | 3192 |
| 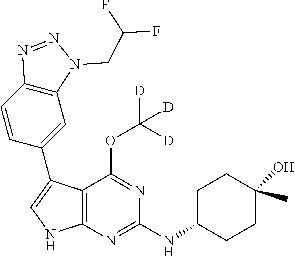 [SMILES: [2H]C([2H])([2H])Oc1nc(N[C@H]2CC[C@@](C)(O)CC2)nc2[nH]cc(-c3ccc4nnn(CC(F)F)c4c3)c12] | 3193 |
| 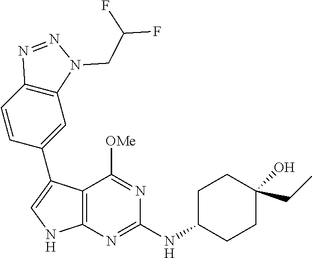 [SMILES: CC[C@]1(O)CC[C@@H](Nc2nc(OC)c3c(-c4ccc5nnn(CC(F)F)c5c4)c[nH]c3n2)CC1] | 3194 |
| 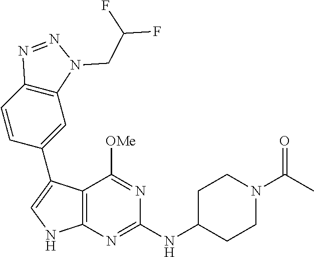 [SMILES: COc1nc(NC2CCN(C(C)=O)CC2)nc2[nH]cc(-c3ccc4nnn(CC(F)F)c4c3)c12] | 3195 |
| 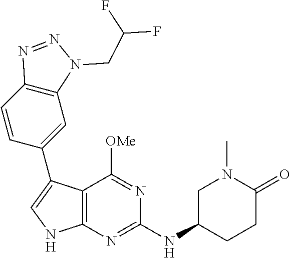 [SMILES: COc1nc(N[C@@H]2CCC(=O)N(C)C2)nc2[nH]cc(-c3ccc4nnn(CC(F)F)c4c3)c12] | 3196 |
| 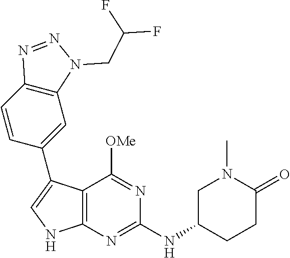 [SMILES: COc1nc(N[C@H]2CCC(=O)N(C)C2)nc2[nH]cc(-c3ccc4nnn(CC(F)F)c4c3)c12] | 3197 |
| 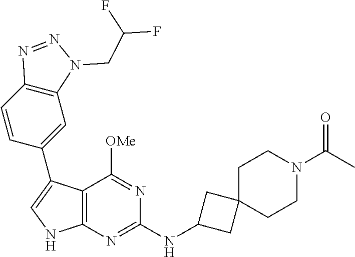 [SMILES: COc1nc(NC2CC3(CCN(C(C)=O)CC3)C2)nc2[nH]cc(-c3ccc4nnn(CC(F)F)c4c3)c12] | 3198 |
| 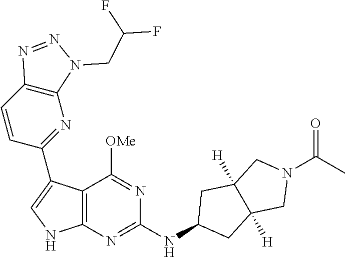 [SMILES: COc1nc(N[C@@H]2C[C@@H]3CN(C(C)=O)C[C@@H]3C2)nc2[nH]cc(-c3ccc4nnn(CC(F)F)c4n3)c12] | 3199 |
| 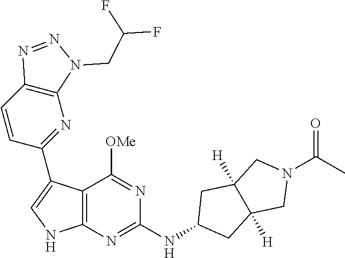 [SMILES: COc1nc(N[C@H]2C[C@@H]3CN(C(C)=O)C[C@@H]3C2)nc2[nH]cc(-c3ccc4nnn(CC(F)F)c4n3)c12] | 3200 |
| 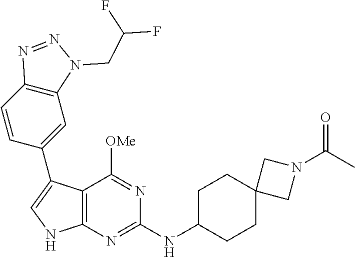 [SMILES: COc1nc(NC2CCC3(CC2)CN(C(C)=O)C3)nc2[nH]cc(-c3ccc4nnn(CC(F)F)c4c3)c12] | 3201 |
| 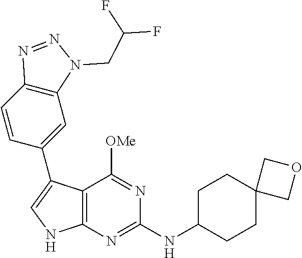 [SMILES: COc1nc(NC2CCC3(CC2)COC3)nc2[nH]cc(-c3ccc4nnn(CC(F)F)c4c3)c12] | 3202 |
| 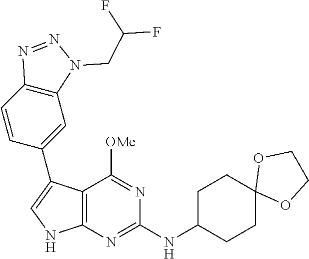 [SMILES: COc1nc(NC2CCC3(CC2)OCCO3)nc2[nH]cc(-c3ccc4nnn(CC(F)F)c4c3)c12] | 3203 |
| 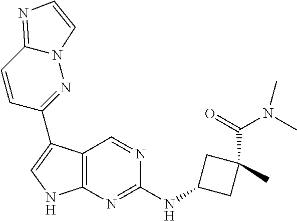 [SMILES: CN(C)C(=O)[C@]1(C)C[C@H](Nc2ncc3c(-c4ccc5nccn5n4)c[nH]c3n2)C1] | 3204 |
| 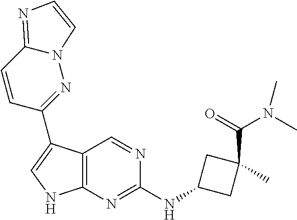 [SMILES: CN(C)C(=O)[C@]1(C)C[C@@H](Nc2ncc3c(-c4ccc5nccn5n4)c[nH]c3n2)C1] | 3205 |
| 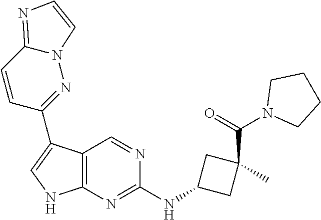 [SMILES: C[C@]1(C(=O)N2CCCC2)C[C@H](Nc2ncc3c(-c4ccc5nccn5n4)c[nH]c3n2)C1] | 3206 |
| 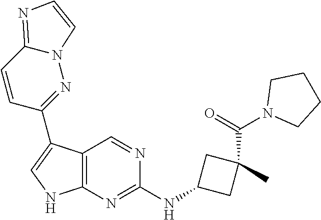 [SMILES: C[C@]1(C(=O)N2CCCC2)C[C@@H](Nc2ncc3c(-c4ccc5nccn5n4)c[nH]c3n2)C1] | 3207 |
| 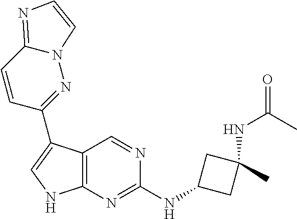 [SMILES: CC(=O)N[C@]1(C)C[C@H](Nc2ncc3c(-c4ccc5nccn5n4)c[nH]c3n2)C1] | 3208 |
| 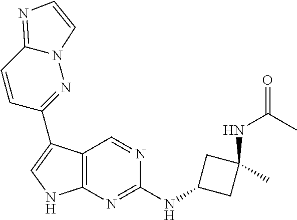 [SMILES: CC(=O)N[C@]1(C)C[C@@H](Nc2ncc3c(-c4ccc5nccn5n4)c[nH]c3n2)C1] | 3209 |
| 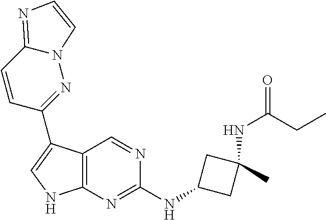 [SMILES: CCC(=O)N[C@]1(C)C[C@H](Nc2ncc3c(-c4ccc5nccn5n4)c[nH]c3n2)C1] | 3210 |
| 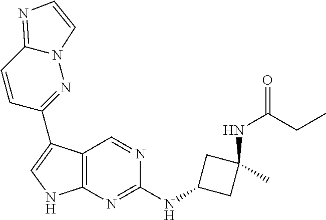 [SMILES: CCC(=O)N[C@]1(C)C[C@@H](Nc2ncc3c(-c4ccc5nccn5n4)c[nH]c3n2)C1] | 3211 |
| 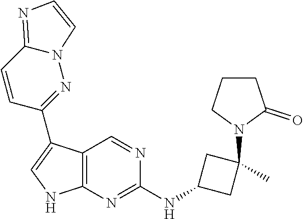 [SMILES: C[C@]1(N2CCCC2=O)C[C@H](Nc2ncc3c(-c4ccc5nccn5n4)c[nH]c3n2)C1] | 3212 |
| 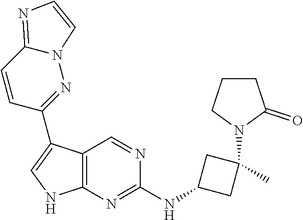 [SMILES: C[C@]1(N2CCCC2=O)C[C@@H](Nc2ncc3c(-c4ccc5nccn5n4)c[nH]c3n2)C1] | 3213 |
| 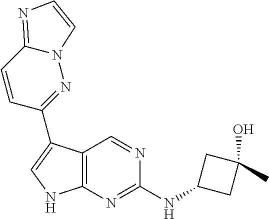 [SMILES: C[C@]1(O)C[C@@H](Nc2ncc3c(-c4ccc5nccn5n4)c[nH]c3n2)C1] | 3214 |
| 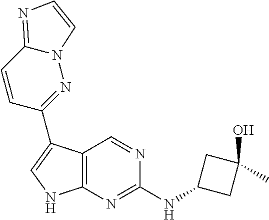 [SMILES: C[C@]1(O)C[C@H](Nc2ncc3c(-c4ccc5nccn5n4)c[nH]c3n2)C1] | 3215 |
| 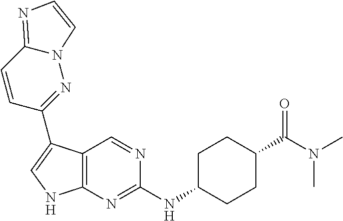 [SMILES: CN(C)C(=O)[C@H]1CC[C@@H](Nc2ncc3c(-c4ccc5nccn5n4)c[nH]c3n2)CC1] | 3216 |
| 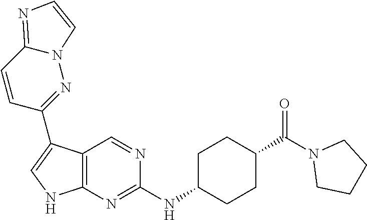 [SMILES: O=C([C@H]1CC[C@@H](Nc2ncc3c(-c4ccc5nccn5n4)c[nH]c3n2)CC1)N1CCCC1] | 3217 |
| 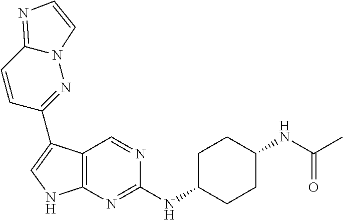 [SMILES: CC(=O)N[C@H]1CC[C@@H](Nc2ncc3c(-c4ccc5nccn5n4)c[nH]c3n2)CC1] | 3218 |
| 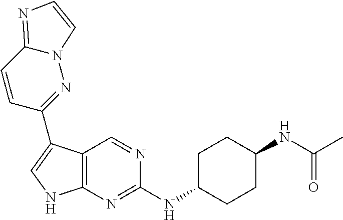 [SMILES: CC(=O)N[C@H]1CC[C@H](Nc2ncc3c(-c4ccc5nccn5n4)c[nH]c3n2)CC1] | 3219 |
| 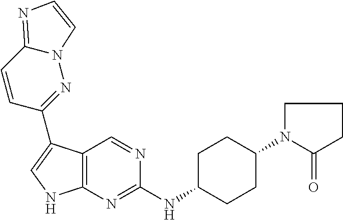 [SMILES: O=C1CCCN1[C@H]1CC[C@@H](Nc2ncc3c(-c4ccc5nccn5n4)c[nH]c3n2)CC1] | 3220 |
| 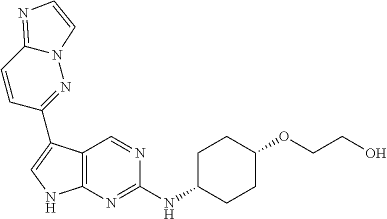 [SMILES: OCCO[C@H]1CC[C@@H](Nc2ncc3c(-c4ccc5nccn5n4)c[nH]c3n2)CC1] | 3221 |
| 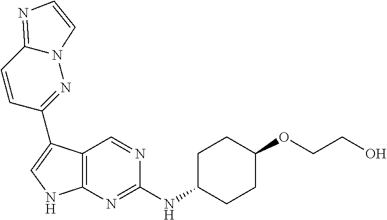 [SMILES: OCCO[C@H]1CC[C@H](Nc2ncc3c(-c4ccc5nccn5n4)c[nH]c3n2)CC1] | 3222 |
| 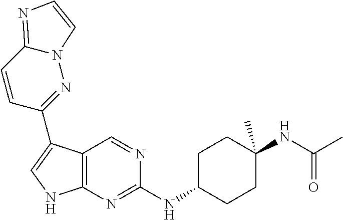 [SMILES: CC(=O)N[C@]1(C)CC[C@@H](Nc2ncc3c(-c4ccc5nccn5n4)c[nH]c3n2)CC1] | 3223 |
| 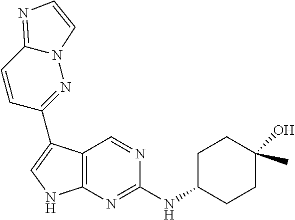 [SMILES: C[C@]1(O)CC[C@@H](Nc2ncc3c(-c4ccc5nccn5n4)c[nH]c3n2)CC1] | 3224 |
| 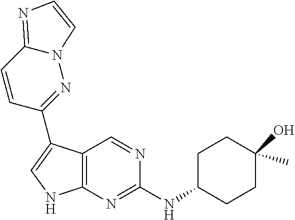 [SMILES: C[C@]1(O)CC[C@H](Nc2ncc3c(-c4ccc5nccn5n4)c[nH]c3n2)CC1] | 3225 |
| 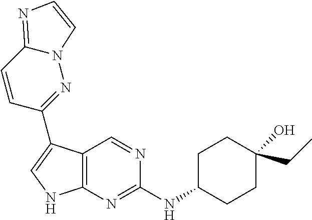 [SMILES: CC[C@]1(O)CC[C@@H](Nc2ncc3c(-c4ccc5nccn5n4)c[nH]c3n2)CC1] | 3226 |
| 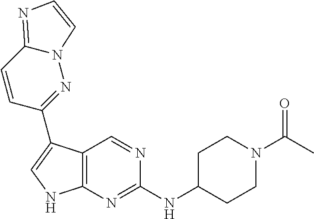 [SMILES: CC(=O)N1CCC(Nc2ncc3c(-c4ccc5nccn5n4)c[nH]c3n2)CC1] | 3227 |
| 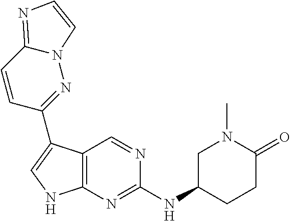 [SMILES: CN1C[C@H](Nc2ncc3c(-c4ccc5nccn5n4)c[nH]c3n2)CCC1=O] | 3228 |
| 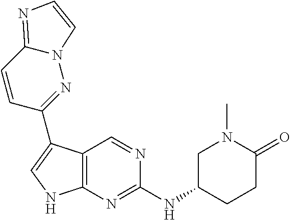 [SMILES: CN1C[C@@H](Nc2ncc3c(-c4ccc5nccn5n4)c[nH]c3n2)CCC1=O] | 3229 |
| 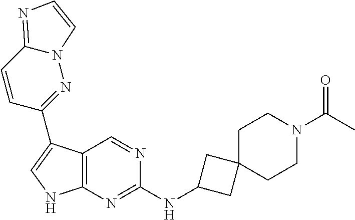 [SMILES: CC(=O)N1CCC2(CC1)CC(Nc1ncc3c(-c4ccc5nccn5n4)c[nH]c3n1)C2] | 3230 |
| 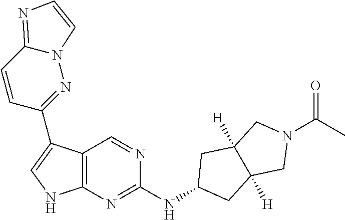 [SMILES: CC(=O)N1C[C@H]2C[C@H](Nc3ncc4c(-c5ccc6nccn6n5)c[nH]c4n3)C[C@H]2C1] | 3231 |
| 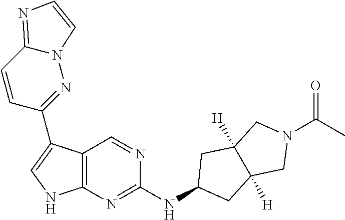 [SMILES: CC(=O)N1C[C@H]2C[C@@H](Nc3ncc4c(-c5ccc6nccn6n5)c[nH]c4n3)C[C@H]2C1] | 3232 |
| 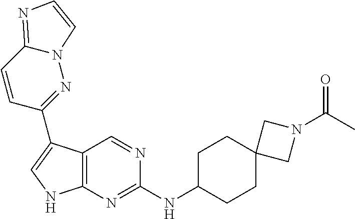 [SMILES: CC(=O)N1CC2(CCC(Nc3ncc4c(-c5ccc6nccn6n5)c[nH]c4n3)CC2)C1] | 3233 |
| 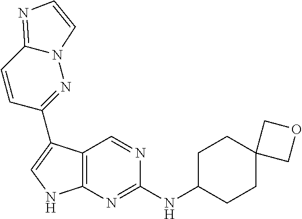 [SMILES: c1cn2nc(-c3c[nH]c4nc(NC5CCC6(CC5)COC6)ncc34)ccc2n1] | 3234 |
| 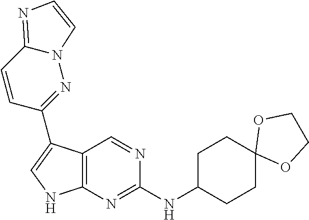 [SMILES: c1cn2nc(-c3c[nH]c4nc(NC5CCC6(CC5)OCCO6)ncc34)ccc2n1] | 3235 |
| 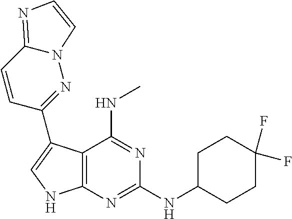 [SMILES: CNc1nc(NC2CCC(F)(F)CC2)nc2[nH]cc(-c3ccc4nccn4n3)c12] | 3236 |
| 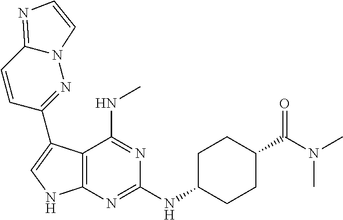 [SMILES: CNc1nc(N[C@H]2CC[C@@H](C(=O)N(C)C)CC2)nc2[nH]cc(-c3ccc4nccn4n3)c12] | 3237 |
| 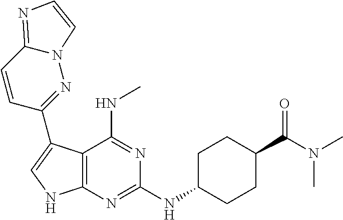 [SMILES: CNc1nc(N[C@H]2CC[C@H](C(=O)N(C)C)CC2)nc2[nH]cc(-c3ccc4nccn4n3)c12] | 3238 |
| 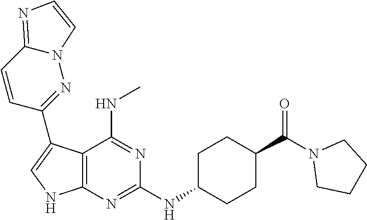 [SMILES: CNc1nc(N[C@H]2CC[C@H](C(=O)N3CCCC3)CC2)nc2[nH]cc(-c3ccc4nccn4n3)c12] | 3239 |
| 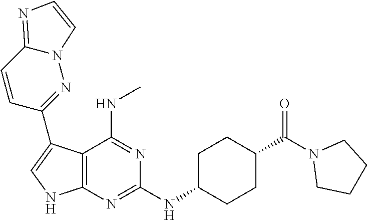 [SMILES: CNc1nc(N[C@H]2CC[C@@H](C(=O)N3CCCC3)CC2)nc2[nH]cc(-c3ccc4nccn4n3)c12] | 3240 |
| 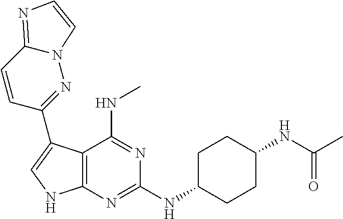 [SMILES: CNc1nc(N[C@H]2CC[C@@H](NC(C)=O)CC2)nc2[nH]cc(-c3ccc4nccn4n3)c12] | 3241 |
| 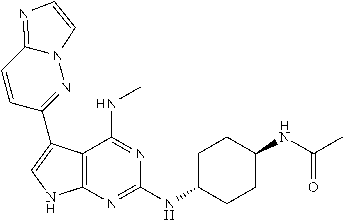 [SMILES: CNc1nc(N[C@H]2CC[C@H](NC(C)=O)CC2)nc2[nH]cc(-c3ccc4nccn4n3)c12] | 3242 |
| 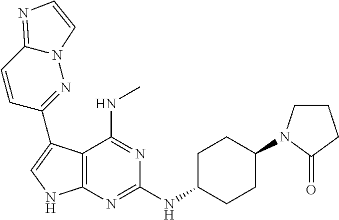 [SMILES: CNc1nc(N[C@H]2CC[C@H](N3CCCC3=O)CC2)nc2[nH]cc(-c3ccc4nccn4n3)c12] | 3243 |
| 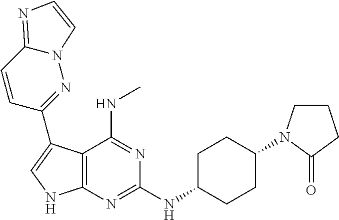 [SMILES: CNc1nc(N[C@H]2CC[C@@H](N3CCCC3=O)CC2)nc2[nH]cc(-c3ccc4nccn4n3)c12] | 3244 |
| 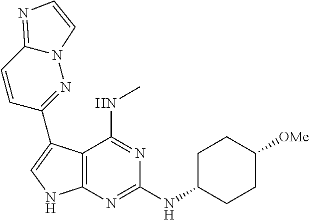 [SMILES: CNc1nc(N[C@H]2CC[C@@H](OC)CC2)nc2[nH]cc(-c3ccc4nccn4n3)c12] | 3245 |
| 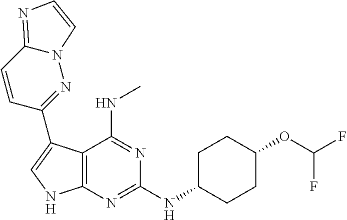 [SMILES: CNc1nc(N[C@H]2CC[C@@H](OC(F)F)CC2)nc2[nH]cc(-c3ccc4nccn4n3)c12] | 3246 |
| 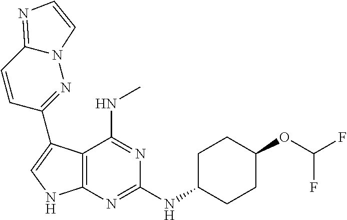 [SMILES: CNc1nc(N[C@H]2CC[C@H](OC(F)F)CC2)nc2[nH]cc(-c3ccc4nccn4n3)c12] | 3247 |
| 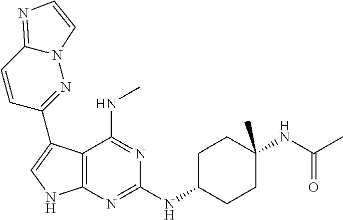 [SMILES: CNc1nc(N[C@H]2CC[C@@](C)(NC(C)=O)CC2)nc2[nH]cc(-c3ccc4nccn4n3)c12] | 3248 |
| 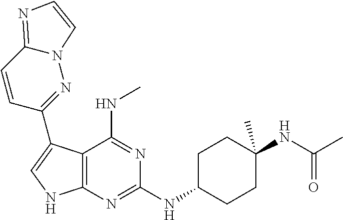 [SMILES: CNc1nc(N[C@H]2CC[C@](C)(NC(C)=O)CC2)nc2[nH]cc(-c3ccc4nccn4n3)c12] | 3249 |
| 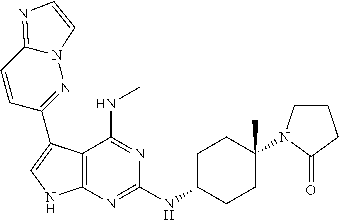 [SMILES: CNc1nc(N[C@H]2CC[C@@](C)(N3CCCC3=O)CC2)nc2[nH]cc(-c3ccc4nccn4n3)c12] | 3250 |
| 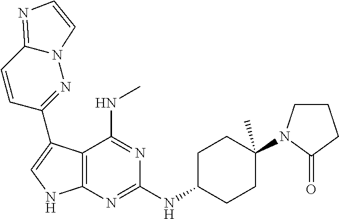 [SMILES: CNc1nc(N[C@H]2CC[C@](C)(N3CCCC3=O)CC2)nc2[nH]cc(-c3ccc4nccn4n3)c12] | 3251 |
| 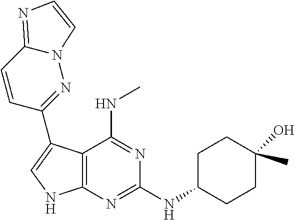 [SMILES: CNc1nc(N[C@H]2CC[C@@](C)(O)CC2)nc2[nH]cc(-c3ccc4nccn4n3)c12] | 3252 |
| 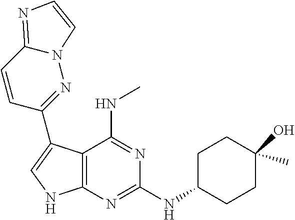 [SMILES: CNc1nc(N[C@H]2CC[C@](C)(O)CC2)nc2[nH]cc(-c3ccc4nccn4n3)c12] | 3253 |
| 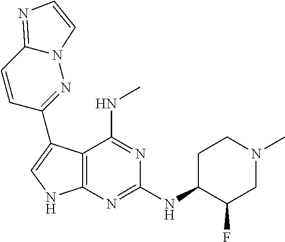 [SMILES: CNc1nc(N[C@H]2CCN(C)C[C@H]2F)nc2[nH]cc(-c3ccc4nccn4n3)c12] | 3254 |
| 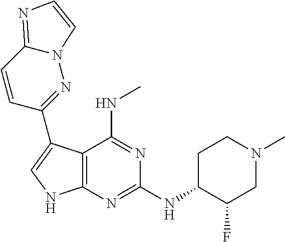 [SMILES: CNc1nc(N[C@@H]2CCN(C)C[C@@H]2F)nc2[nH]cc(-c3ccc4nccn4n3)c12] | 3255 |
| 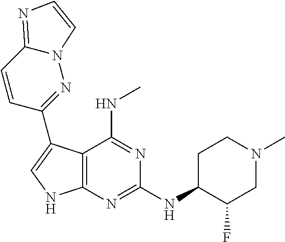 [SMILES: CNc1nc(N[C@H]2CCN(C)C[C@@H]2F)nc2[nH]cc(-c3ccc4nccn4n3)c12] | 3256 |
| 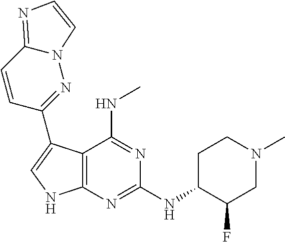 [SMILES: CNc1nc(N[C@@H]2CCN(C)C[C@H]2F)nc2[nH]cc(-c3ccc4nccn4n3)c12] | 3257 |
| 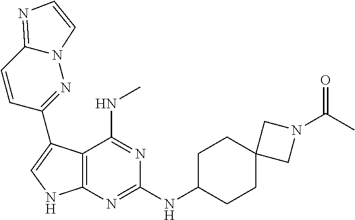 [SMILES: CNc1nc(NC2CCC3(CC2)CN(C(C)=O)C3)nc2[nH]cc(-c3ccc4nccn4n3)c12] | 3258 |
| 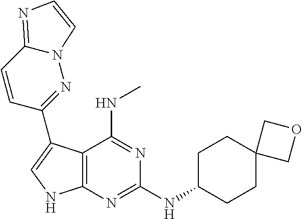 | 3259 |
| 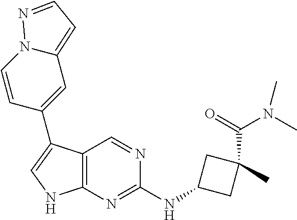 [SMILES: CN(C)C(=O)[C@]1(C)C[C@H](Nc2ncc3c(-c4ccn5nccc5c4)c[nH]c3n2)C1] | 3260 |
| 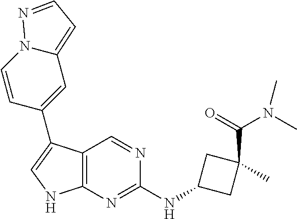 [SMILES: CN(C)C(=O)[C@]1(C)C[C@@H](Nc2ncc3c(-c4ccn5nccc5c4)c[nH]c3n2)C1] | 3261 |
| 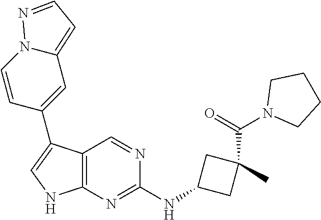 [SMILES: C[C@]1(C(=O)N2CCCC2)C[C@@H](Nc2ncc3c(-c4ccn5nccc5c4)c[nH]c3n2)C1] | 3262 |
| 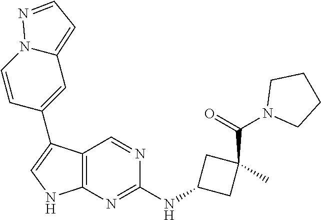 [SMILES: C[C@]1(C(=O)N2CCCC2)C[C@H](Nc2ncc3c(-c4ccn5nccc5c4)c[nH]c3n2)C1] | 3263 |
| 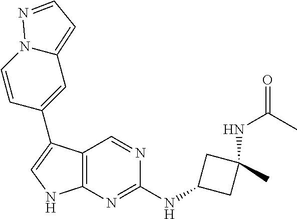 [SMILES: CC(=O)N[C@]1(C)C[C@H](Nc2ncc3c(-c4ccn5nccc5c4)c[nH]c3n2)C1] | 3264 |
| 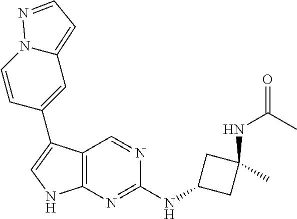 [SMILES: CC(=O)N[C@]1(C)C[C@@H](Nc2ncc3c(-c4ccn5nccc5c4)c[nH]c3n2)C1] | 3265 |
| 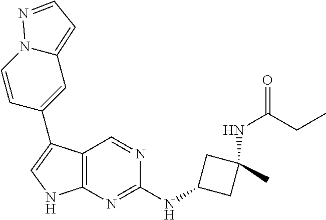 [SMILES: CCC(=O)N[C@]1(C)C[C@H](Nc2ncc3c(-c4ccn5nccc5c4)c[nH]c3n2)C1] | 3266 |
| 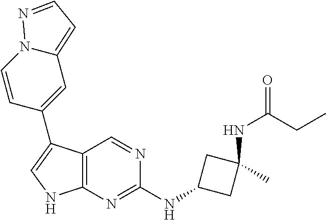 [SMILES: CCC(=O)N[C@]1(C)C[C@@H](Nc2ncc3c(-c4ccn5nccc5c4)c[nH]c3n2)C1] | 3267 |
| 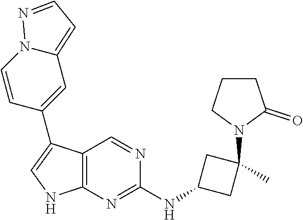 [SMILES: C[C@]1(N2CCCC2=O)C[C@H](Nc2ncc3c(-c4ccn5nccc5c4)c[nH]c3n2)C1] | 3268 |
| 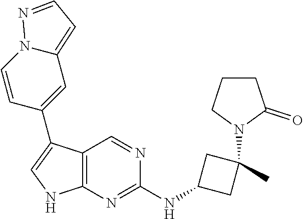 [SMILES: C[C@]1(N2CCCC2=O)C[C@@H](Nc2ncc3c(-c4ccn5nccc5c4)c[nH]c3n2)C1] | 3269 |
| 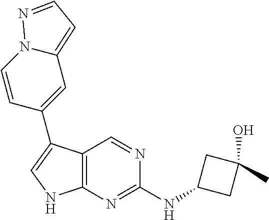 [SMILES: C[C@]1(O)C[C@@H](Nc2ncc3c(-c4ccn5nccc5c4)c[nH]c3n2)C1] | 3270 |
| 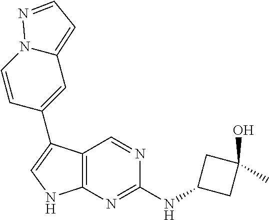 [SMILES: C[C@]1(O)C[C@H](Nc2ncc3c(-c4ccn5nccc5c4)c[nH]c3n2)C1] | 3271 |
| 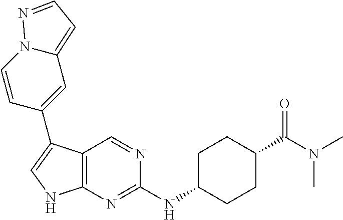 [SMILES: CN(C)C(=O)[C@H]1CC[C@@H](Nc2ncc3c(-c4ccn5nccc5c4)c[nH]c3n2)CC1] | 3272 |
| 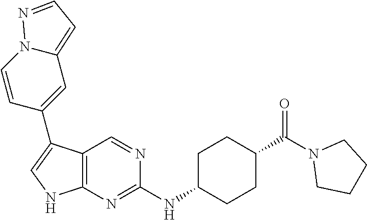 [SMILES: O=C([C@H]1CC[C@@H](Nc2ncc3c(-c4ccn5nccc5c4)c[nH]c3n2)CC1)N1CCCC1] | 3273 |
| 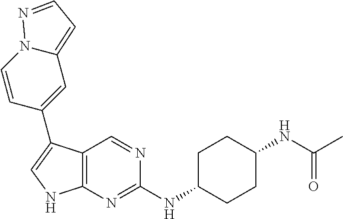 [SMILES: CC(=O)N[C@H]1CC[C@@H](Nc2ncc3c(-c4ccn5nccc5c4)c[nH]c3n2)CC1] | 3274 |
| 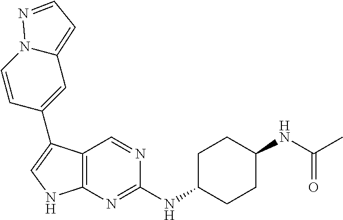 [SMILES: CC(=O)N[C@H]1CC[C@H](Nc2ncc3c(-c4ccn5nccc5c4)c[nH]c3n2)CC1] | 3275 |
| 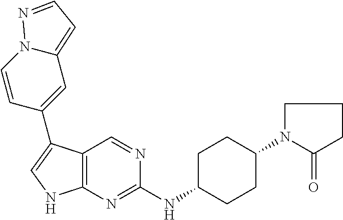 [SMILES: O=C1CCCN1[C@H]1CC[C@@H](Nc2ncc3c(-c4ccn5nccc5c4)c[nH]c3n2)CC1] | 3276 |
| 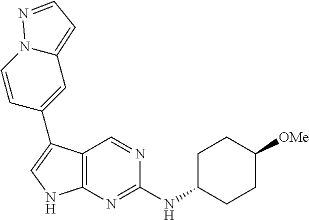 [SMILES: CO[C@H]1CC[C@H](Nc2ncc3c(-c4ccn5nccc5c4)c[nH]c3n2)CC1] | 3277 |
| 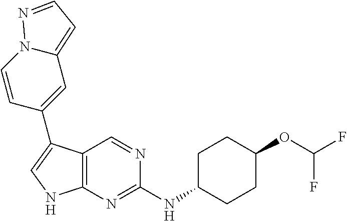 [SMILES: FC(F)O[C@H]1CC[C@H](Nc2ncc3c(-c4ccn5nccc5c4)c[nH]c3n2)CC1] | 3278 |
| 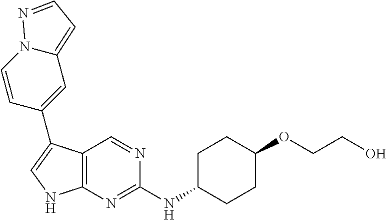 [SMILES: OCCO[C@H]1CC[C@H](Nc2ncc3c(-c4ccn5nccc5c4)c[nH]c3n2)CC1] | 3279 |
| 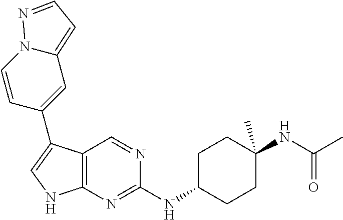 [SMILES: CC(=O)N[C@]1(C)CC[C@@H](Nc2ncc3c(-c4ccn5nccc5c4)c[nH]c3n2)CC1] | 3280 |
| 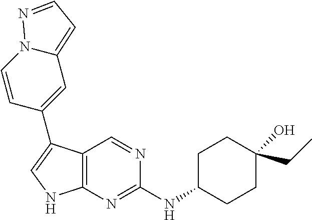 [SMILES: CC[C@]1(O)CC[C@@H](Nc2ncc3c(-c4ccn5nccc5c4)c[nH]c3n2)CC1] | 3281 |
| 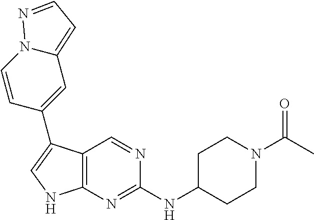 [SMILES: CC(=O)N1CCC(Nc2ncc3c(-c4ccn5nccc5c4)c[nH]c3n2)CC1] | 3282 |
| 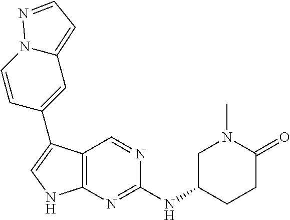 [SMILES: CN1C[C@@H](Nc2ncc3c(-c4ccn5nccc5c4)c[nH]c3n2)CCC1=O] | 3283 |
| 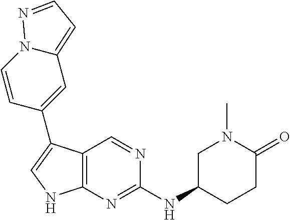 [SMILES: CN1C[C@H](Nc2ncc3c(-c4ccn5nccc5c4)c[nH]c3n2)CCC1=O] | 3284 |
| 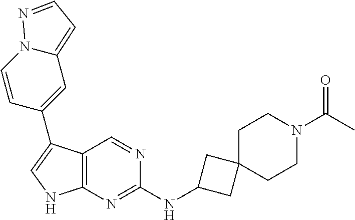 [SMILES: CC(=O)N1CCC2(CC1)CC(Nc1ncc3c(-c4ccn5nccc5c4)c[nH]c3n1)C2] | 3285 |
| 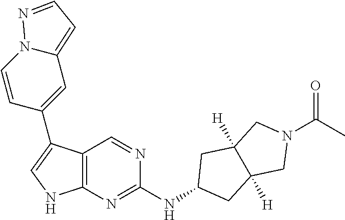 [SMILES: CC(=O)N1C[C@H]2C[C@H](Nc3ncc4c(-c5ccn6nccc6c5)c[nH]c4n3)C[C@H]2C1] | 3286 |
| 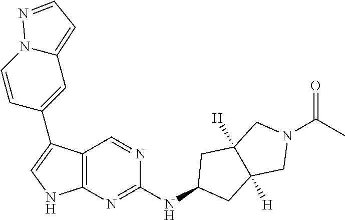 [SMILES: CC(=O)N1C[C@H]2C[C@@H](Nc3ncc4c(-c5ccn6nccc6c5)c[nH]c4n3)C[C@H]2C1] | 3287 |
| 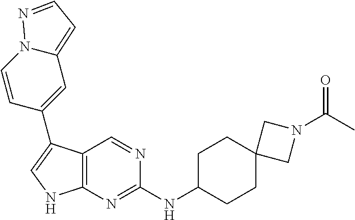 [SMILES: CC(=O)N1CC2(CCC(Nc3ncc4c(-c5ccn6nccc6c5)c[nH]c4n3)CC2)C1] | 3288 |
| 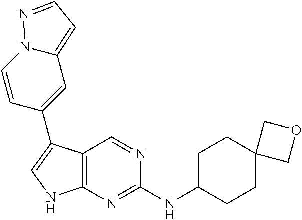 [SMILES: c1cc2cc(-c3c[nH]c4nc(NC5CCC6(CC5)COC6)ncc34)ccn2n1] | 3289 |
| 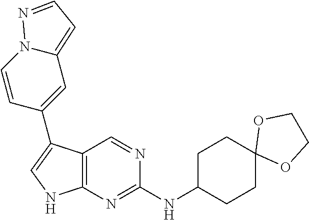 [SMILES: c1cc2cc(-c3c[nH]c4nc(NC5CCC6(CC5)OCCO6)ncc34)ccn2n1] | 3290 |
| 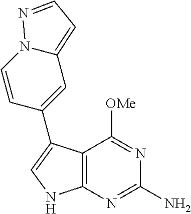 [SMILES: COc1nc(N)nc2[nH]cc(-c3ccn4nccc4c3)c12] | 3291 |
| 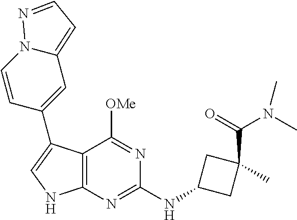 [SMILES: COc1nc(N[C@H]2C[C@](C)(C(=O)N(C)C)C2)nc2[nH]cc(-c3ccn4nccc4c3)c12] | 3292 |
| 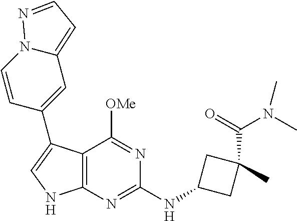 [SMILES: COc1nc(N[C@H]2C[C@@](C)(C(=O)N(C)C)C2)nc2[nH]cc(-c3ccn4nccc4c3)c12] | 3293 |
| 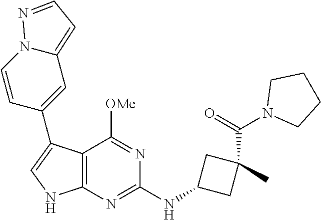 [SMILES: COc1nc(N[C@H]2C[C@@](C)(C(=O)N3CCCC3)C2)nc2[nH]cc(-c3ccn4nccc4c3)c12] | 3294 |
| 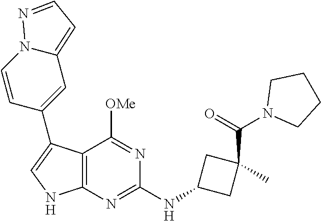 [SMILES: COc1nc(N[C@H]2C[C@](C)(C(=O)N3CCCC3)C2)nc2[nH]cc(-c3ccn4nccc4c3)c12] | 3295 |
| 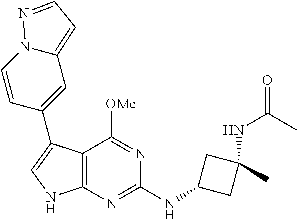 [SMILES: COc1nc(N[C@H]2C[C@@](C)(NC(C)=O)C2)nc2[nH]cc(-c3ccn4nccc4c3)c12] | 3296 |
| 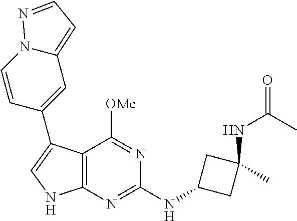 [SMILES: COc1nc(N[C@H]2C[C@](C)(NC(C)=O)C2)nc2[nH]cc(-c3ccn4nccc4c3)c12] | 3297 |
| 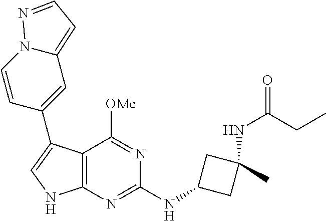 [SMILES: CCC(=O)N[C@]1(C)C[C@H](Nc2nc(OC)c3c(-c4ccn5nccc5c4)c[nH]c3n2)C1] | 3298 |
| 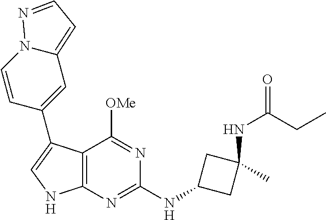 [SMILES: CCC(=O)N[C@]1(C)C[C@@H](Nc2nc(OC)c3c(-c4ccn5nccc5c4)c[nH]c3n2)C1] | 3299 |
| 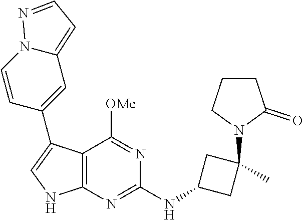 [SMILES: COc1nc(N[C@H]2C[C@](C)(N3CCCC3=O)C2)nc2[nH]cc(-c3ccn4nccc4c3)c12] | 3300 |
| 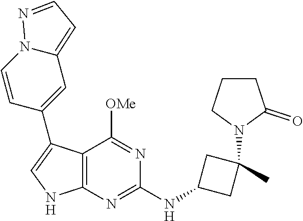 [SMILES: COc1nc(N[C@H]2C[C@@](C)(N3CCCC3=O)C2)nc2[nH]cc(-c3ccn4nccc4c3)c12] | 3301 |
| 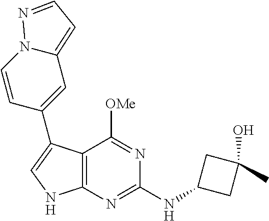 [SMILES: COc1nc(N[C@H]2C[C@@](C)(O)C2)nc2[nH]cc(-c3ccn4nccc4c3)c12] | 3302 |
| 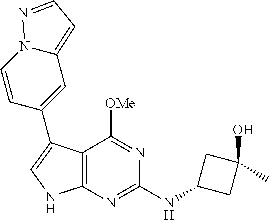 [SMILES: COc1nc(N[C@H]2C[C@](C)(O)C2)nc2[nH]cc(-c3ccn4nccc4c3)c12] | 3303 |
| 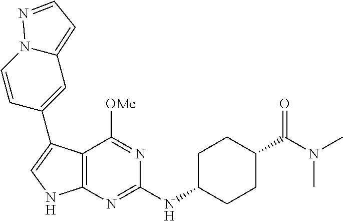 [SMILES: COc1nc(N[C@H]2CC[C@@H](C(=O)N(C)C)CC2)nc2[nH]cc(-c3ccn4nccc4c3)c12] | 3304 |
| 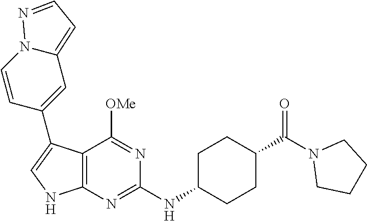 [SMILES: COc1nc(N[C@H]2CC[C@@H](C(=O)N3CCCC3)CC2)nc2[nH]cc(-c3ccn4nccc4c3)c12] | 3305 |
| 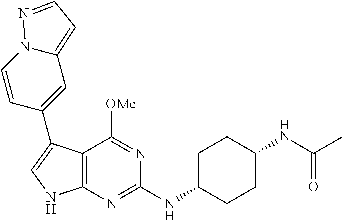 [SMILES: COc1nc(N[C@H]2CC[C@@H](NC(C)=O)CC2)nc2[nH]cc(-c3ccn4nccc4c3)c12] | 3306 |
| 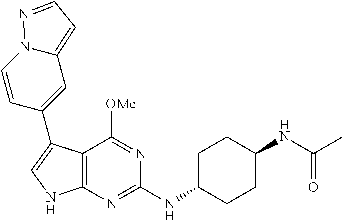 [SMILES: COc1nc(N[C@H]2CC[C@H](NC(C)=O)CC2)nc2[nH]cc(-c3ccn4nccc4c3)c12] | 3307 |
| 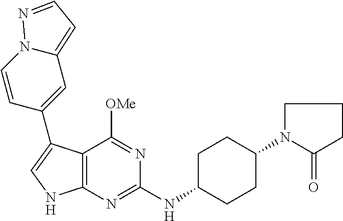 [SMILES: COc1nc(N[C@H]2CC[C@@H](N3CCCC3=O)CC2)nc2[nH]cc(-c3ccn4nccc4c3)c12] | 3308 |
| 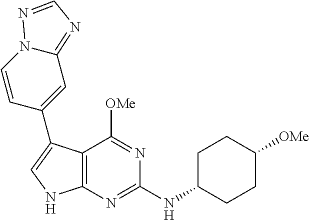 [SMILES: COc1nc(N[C@H]2CC[C@@H](OC)CC2)nc2[nH]cc(-c3ccn4ncnc4c3)c12] | 3309 |
| 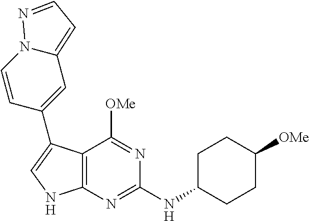 [SMILES: COc1nc(N[C@H]2CC[C@H](OC)CC2)nc2[nH]cc(-c3ccn4nccc4c3)c12] | 3310 |
| 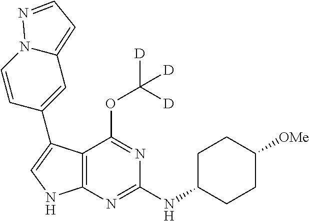 [SMILES: [2H]C([2H])([2H])Oc1nc(N[C@H]2CC[C@@H](OC)CC2)nc2[nH]cc(-c3ccn4nccc4c3)c12] | 3311 |
| 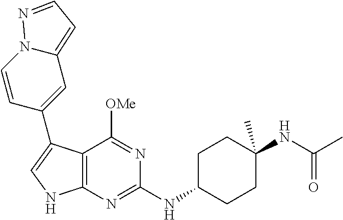 [SMILES: COc1nc(N[C@H]2CC[C@](C)(NC(C)=O)CC2)nc2[nH]cc(-c3ccn4nccc4c3)c12] | 3312 |
| 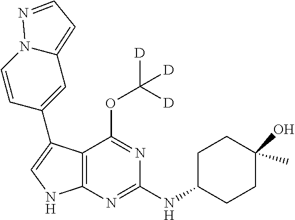 [SMILES: [2H]C([2H])([2H])Oc1nc(N[C@H]2CC[C@](C)(O)CC2)nc2[nH]cc(-c3ccn4nccc4c3)c12] | 3313 |
| 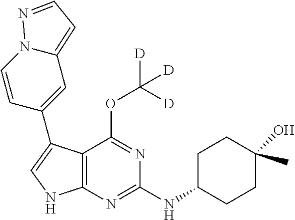 [SMILES: [2H]C([2H])([2H])Oc1nc(N[C@H]2CC[C@@](C)(O)CC2)nc2[nH]cc(-c3ccn4nccc4c3)c12] | 3314 |
| 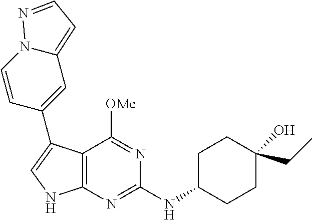 [SMILES: CC[C@]1(O)CC[C@@H](Nc2nc(OC)c3c(-c4ccn5nccc5c4)c[nH]c3n2)CC1] | 3315 |
| 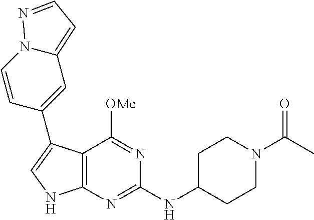 [SMILES: COc1nc(NC2CCN(C(C)=O)CC2)nc2[nH]cc(-c3ccn4nccc4c3)c12] | 3316 |
| 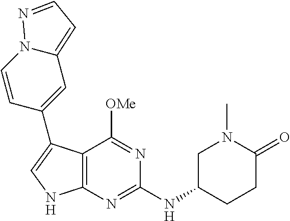 [SMILES: COc1nc(N[C@H]2CCC(=O)N(C)C2)nc2[nH]cc(-c3ccn4nccc4c3)c12] | 3317 |
| 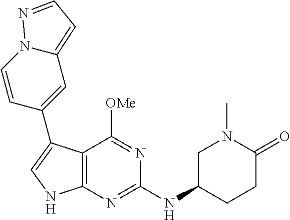 [SMILES: COc1nc(N[C@@H]2CCC(=O)N(C)C2)nc2[nH]cc(-c3ccn4nccc4c3)c12] | 3318 |
| 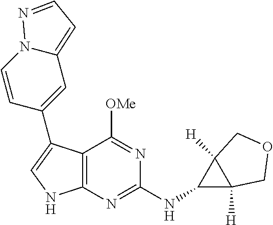 [SMILES: COc1nc(N[C@H]2[C@@H]3COC[C@@H]32)nc2[nH]cc(-c3ccn4nccc4c3)c12] | 3319 |
| 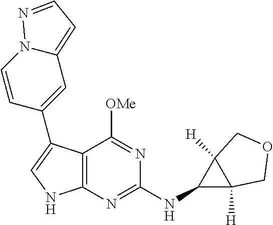 [SMILES: COc1nc(N[C@@H]2[C@@H]3COC[C@@H]32)nc2[nH]cc(-c3ccn4nccc4c3)c12] | 3320 |
| 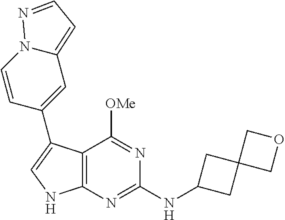 [SMILES: COc1nc(NC2CC3(COC3)C2)nc2[nH]cc(-c3ccn4nccc4c3)c12] | 3321 |
| 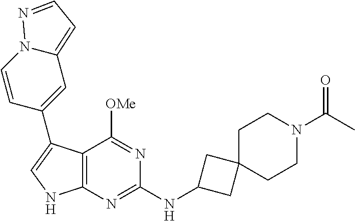 [SMILES: COc1nc(NC2CC3(CCN(C(C)=O)CC3)C2)nc2[nH]cc(-c3ccn4nccc4c3)c12] | 3322 |
| 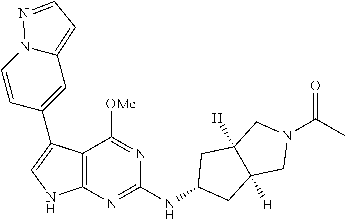 [SMILES: COc1nc(N[C@H]2C[C@@H]3CN(C(C)=O)C[C@@H]3C2)nc2[nH]cc(-c3ccn4nccc4c3)c12] | 3323 |
| 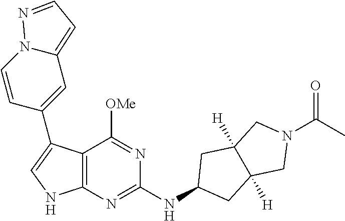 [SMILES: COc1nc(N[C@@H]2C[C@@H]3CN(C(C)=O)C[C@@H]3C2)nc2[nH]cc(-c3ccn4nccc4c3)c12] | 3324 |
| 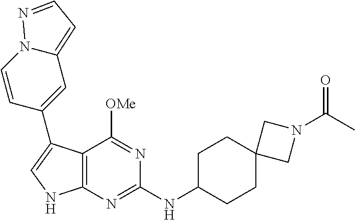 [SMILES: COc1nc(NC2CCC3(CC2)CN(C(C)=O)C3)nc2[nH]cc(-c3ccn4nccc4c3)c12] | 3325 |
| 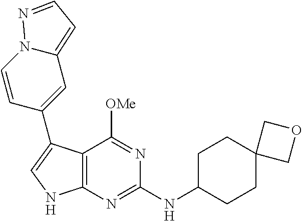 [SMILES: COc1nc(NC2CCC3(CC2)COC3)nc2[nH]cc(-c3ccn4nccc4c3)c12] | 3326 |
| 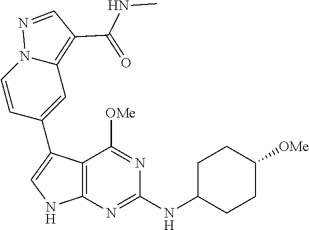 | 3327 |
| 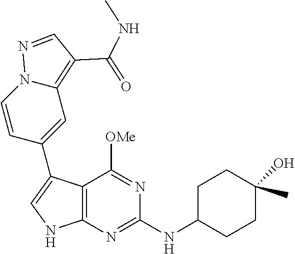 | 3328 |
| 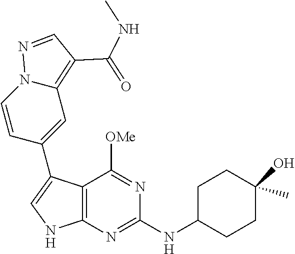 | 3329 |
| 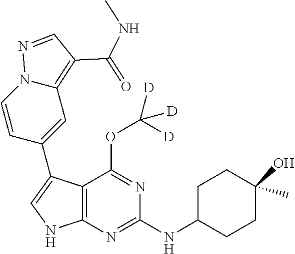 | 3330 |
| 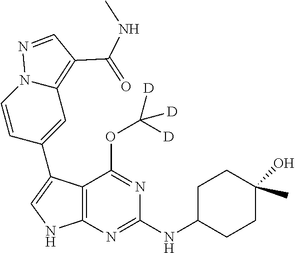 | 3331 |
| 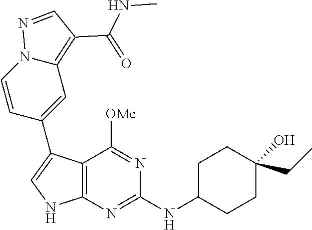 | 3332 |
| 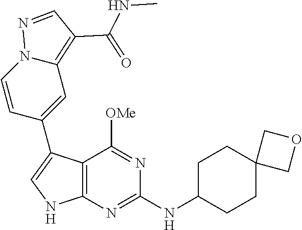 [SMILES: CNC(=O)c1cnn2ccc(-c3c[nH]c4nc(NC5CCC6(CC5)COC6)nc(OC)c34)cc12] | 3333 |
| 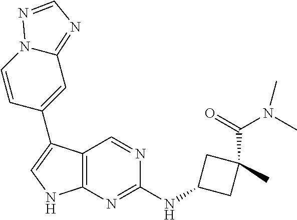 [SMILES: CN(C)C(=O)[C@]1(C)C[C@H](Nc2ncc3c(-c4ccn5ncnc5c4)c[nH]c3n2)C1] | 3334 |
| 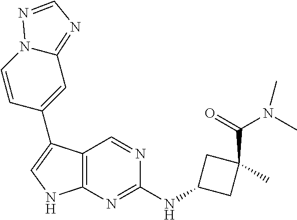 [SMILES: CN(C)C(=O)[C@]1(C)C[C@@H](Nc2ncc3c(-c4ccn5ncnc5c4)c[nH]c3n2)C1] | 3335 |
| 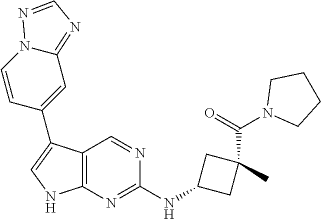 [SMILES: C[C@]1(C(=O)N2CCCC2)C[C@@H](Nc2ncc3c(-c4ccn5ncnc5c4)c[nH]c3n2)C1] | 3336 |
| 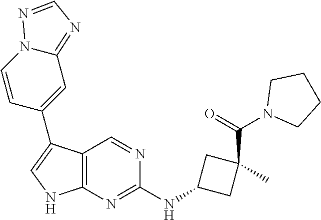 [SMILES: C[C@]1(C(=O)N2CCCC2)C[C@H](Nc2ncc3c(-c4ccn5ncnc5c4)c[nH]c3n2)C1] | 3337 |
| 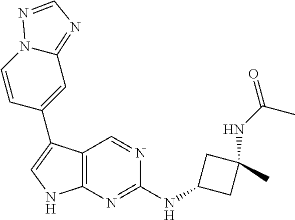 [SMILES: CC(=O)N[C@]1(C)C[C@H](Nc2ncc3c(-c4ccn5ncnc5c4)c[nH]c3n2)C1] | 3338 |
| 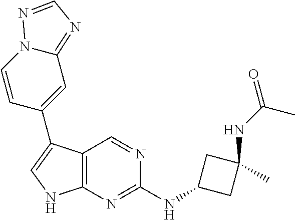 [SMILES: CC(=O)N[C@]1(C)C[C@@H](Nc2ncc3c(-c4ccn5ncnc5c4)c[nH]c3n2)C1] | 3339 |
| 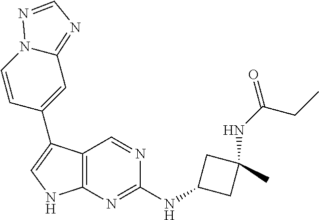 [SMILES: CCC(=O)N[C@]1(C)C[C@H](Nc2ncc3c(-c4ccn5ncnc5c4)c[nH]c3n2)C1] | 3340 |
| 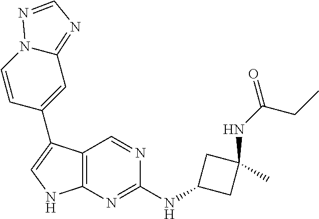 [SMILES: CCC(=O)N[C@]1(C)C[C@@H](Nc2ncc3c(-c4ccn5ncnc5c4)c[nH]c3n2)C1] | 3341 |
| 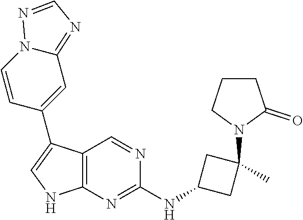 [SMILES: C[C@]1(N2CCCC2=O)C[C@H](Nc2ncc3c(-c4ccn5ncnc5c4)c[nH]c3n2)C1] | 3342 |
| 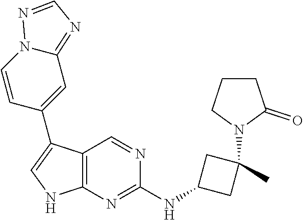 [SMILES: C[C@]1(N2CCCC2=O)C[C@@H](Nc2ncc3c(-c4ccn5ncnc5c4)c[nH]c3n2)C1] | 3343 |
| 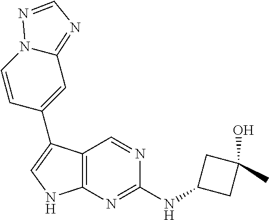 [SMILES: C[C@]1(O)C[C@@H](Nc2ncc3c(-c4ccn5ncnc5c4)c[nH]c3n2)C1] | 3344 |
| 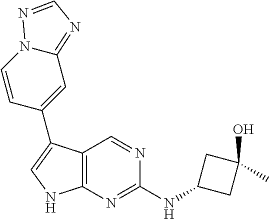 [SMILES: C[C@]1(O)C[C@H](Nc2ncc3c(-c4ccn5ncnc5c4)c[nH]c3n2)C1] | 3345 |
| 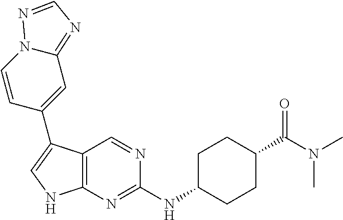 [SMILES: CN(C)C(=O)[C@H]1CC[C@@H](Nc2ncc3c(-c4ccn5ncnc5c4)c[nH]c3n2)CC1] | 3346 |
| 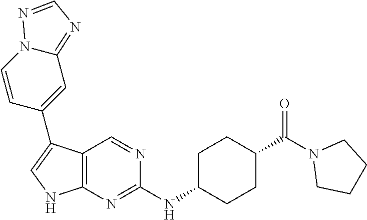 [SMILES: O=C([C@H]1CC[C@@H](Nc2ncc3c(-c4ccn5ncnc5c4)c[nH]c3n2)CC1)N1CCCC1] | 3347 |
| 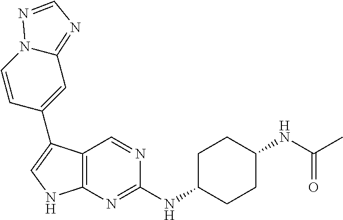 [SMILES: CC(=O)N[C@H]1CC[C@@H](Nc2ncc3c(-c4ccn5ncnc5c4)c[nH]c3n2)CC1] | 3348 |
| 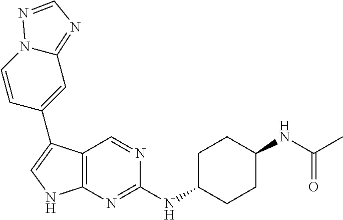 [SMILES: CC(=O)N[C@H]1CC[C@H](Nc2ncc3c(-c4ccn5ncnc5c4)c[nH]c3n2)CC1] | 3349 |
| 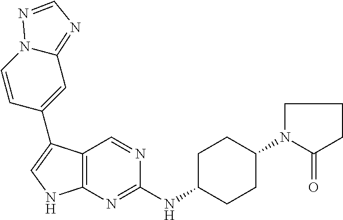 [SMILES: O=C1CCCN1[C@H]1CC[C@@H](Nc2ncc3c(-c4ccn5ncnc5c4)c[nH]c3n2)CC1] | 3350 |
| 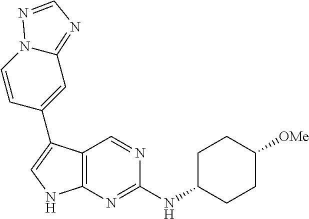 [SMILES: CO[C@H]1CC[C@@H](Nc2ncc3c(-c4ccn5ncnc5c4)c[nH]c3n2)CC1] | 3351 |
| 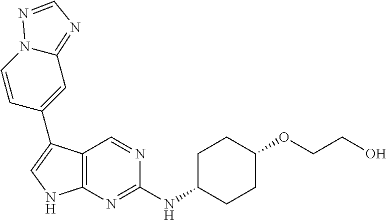 [SMILES: OCCO[C@H]1CC[C@@H](Nc2ncc3c(-c4ccn5ncnc5c4)c[nH]c3n2)CC1] | 3352 |
| 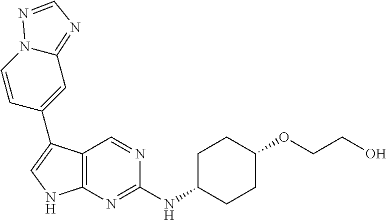 [SMILES: OCCO[C@H]1CC[C@@H](Nc2ncc3c(-c4ccn5ncnc5c4)c[nH]c3n2)CC1] | 3353 |
| 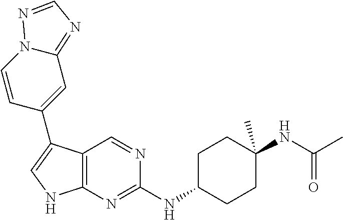 [SMILES: CC(=O)N[C@]1(C)CC[C@@H](Nc2ncc3c(-c4ccn5ncnc5c4)c[nH]c3n2)CC1] | 3354 |
| 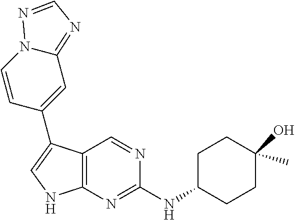 [SMILES: C[C@]1(O)CC[C@H](Nc2ncc3c(-c4ccn5ncnc5c4)c[nH]c3n2)CC1] | 3355 |
| 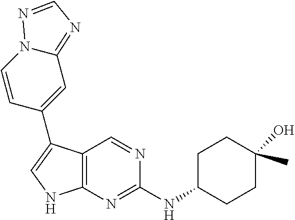 [SMILES: C[C@]1(O)CC[C@@H](Nc2ncc3c(-c4ccn5ncnc5c4)c[nH]c3n2)CC1] | 3356 |
| 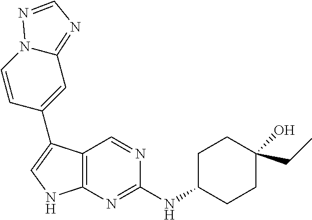 [SMILES: CC[C@]1(O)CC[C@@H](Nc2ncc3c(-c4ccn5ncnc5c4)c[nH]c3n2)CC1] | 3357 |
| 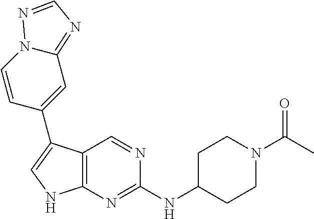 [SMILES: CC(=O)N1CCC(Nc2ncc3c(-c4ccn5ncnc5c4)c[nH]c3n2)CC1] | 3358 |
| 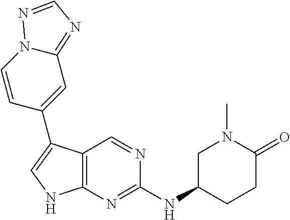 [SMILES: CN1C[C@H](Nc2ncc3c(-c4ccn5ncnc5c4)c[nH]c3n2)CCC1=O] | 3359 |
| 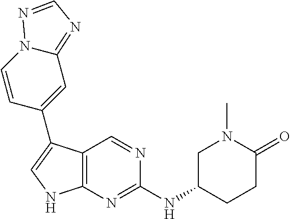 [SMILES: CN1C[C@@H](Nc2ncc3c(-c4ccn5ncnc5c4)c[nH]c3n2)CCC1=O] | 3360 |
| 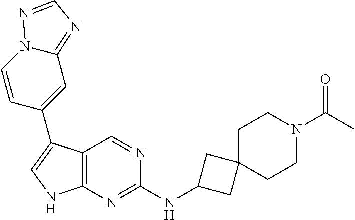 [SMILES: CC(=O)N1CCC2(CC1)CC(Nc1ncc3c(-c4ccn5ncnc5c4)c[nH]c3n1)C2] | 3361 |
| 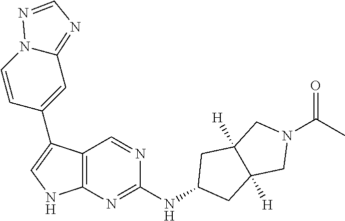 [SMILES: CC(=O)N1C[C@H]2C[C@H](Nc3ncc4c(-c5ccn6ncnc6c5)c[nH]c4n3)C[C@H]2C1] | 3362 |
| 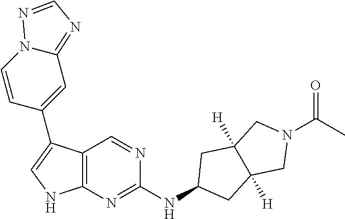 [SMILES: CC(=O)N1C[C@H]2C[C@@H](Nc3ncc4c(-c5ccn6ncnc6c5)c[nH]c4n3)C[C@H]2C1] | 3363 |
| 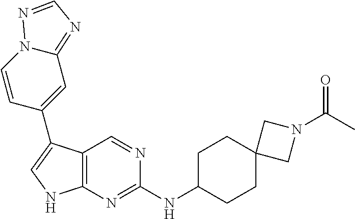 [SMILES: CC(=O)N1CC2(CCC(Nc3ncc4c(-c5ccn6ncnc6c5)c[nH]c4n3)CC2)C1] | 3364 |
| 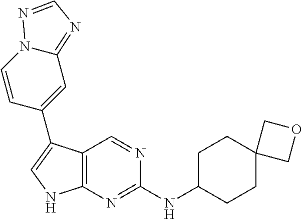 [SMILES: c1nc2cc(-c3c[nH]c4nc(NC5CCC6(CC5)COC6)ncc34)ccn2n1] | 3365 |
| 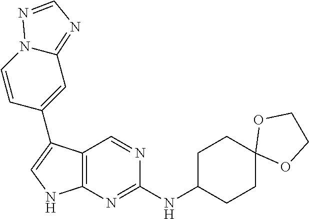 [SMILES: c1nc2cc(-c3c[nH]c4nc(NC5CCC6(CC5)OCCO6)ncc34)ccn2n1] | 3366 |
| 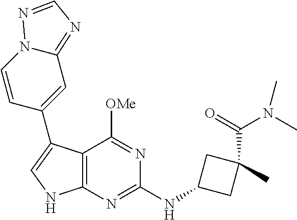 [SMILES: COc1nc(N[C@H]2C[C@@](C)(C(=O)N(C)C)C2)nc2[nH]cc(-c3ccn4ncnc4c3)c12] | 3367 |
| 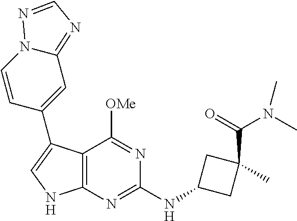 [SMILES: COc1nc(N[C@H]2C[C@](C)(C(=O)N(C)C)C2)nc2[nH]cc(-c3ccn4ncnc4c3)c12] | 3368 |
| 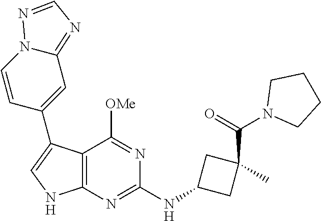 [SMILES: COc1nc(N[C@H]2C[C@](C)(C(=O)N3CCCC3)C2)nc2[nH]cc(-c3ccn4ncnc4c3)c12] | 3369 |
| 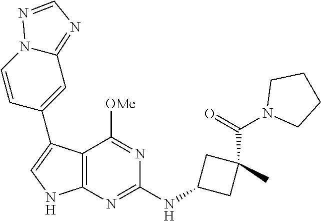 [SMILES: COc1nc(N[C@H]2C[C@@](C)(C(=O)N3CCCC3)C2)nc2[nH]cc(-c3ccn4ncnc4c3)c12] | 3370 |
| 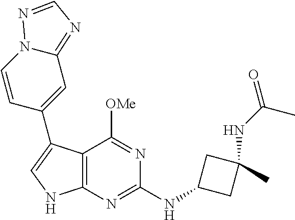 [SMILES: COc1nc(N[C@H]2C[C@@](C)(NC(C)=O)C2)nc2[nH]cc(-c3ccn4ncnc4c3)c12] | 3371 |
| 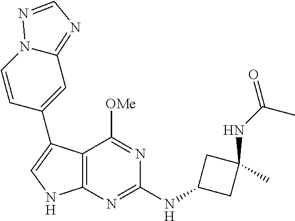 [SMILES: COc1nc(N[C@H]2C[C@](C)(NC(C)=O)C2)nc2[nH]cc(-c3ccn4ncnc4c3)c12] | 3372 |
| 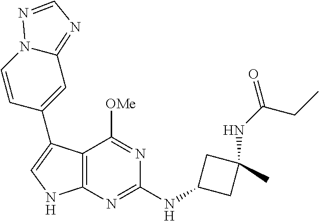 [SMILES: CCC(=O)N[C@]1(C)C[C@H](Nc2nc(OC)c3c(-c4ccn5ncnc5c4)c[nH]c3n2)C1] | 3373 |
| 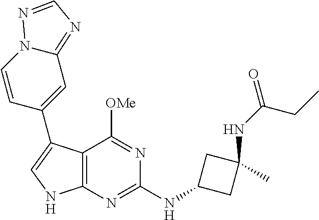 [SMILES: CCC(=O)N[C@]1(C)C[C@@H](Nc2nc(OC)c3c(-c4ccn5ncnc5c4)c[nH]c3n2)C1] | 3374 |
| 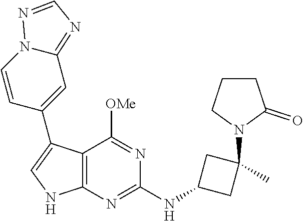 [SMILES: COc1nc(N[C@H]2C[C@](C)(N3CCCC3=O)C2)nc2[nH]cc(-c3ccn4ncnc4c3)c12] | 3375 |
| 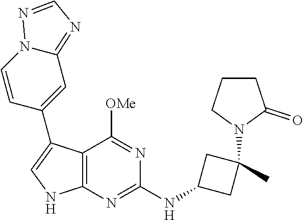 [SMILES: COc1nc(N[C@H]2C[C@@](C)(N3CCCC3=O)C2)nc2[nH]cc(-c3ccn4ncnc4c3)c12] | 3376 |
| 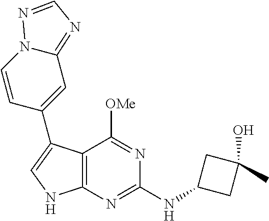 [SMILES: COc1nc(N[C@H]2C[C@@](C)(O)C2)nc2[nH]cc(-c3ccn4ncnc4c3)c12] | 3377 |
| 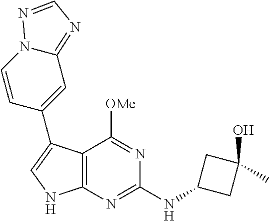 [SMILES: COc1nc(N[C@H]2C[C@](C)(O)C2)nc2[nH]cc(-c3ccn4ncnc4c3)c12] | 3378 |
| 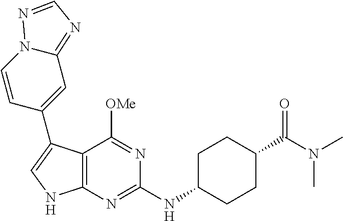 [SMILES: COc1nc(N[C@H]2CC[C@@H](C(=O)N(C)C)CC2)nc2[nH]cc(-c3ccn4ncnc4c3)c12] | 3379 |
| 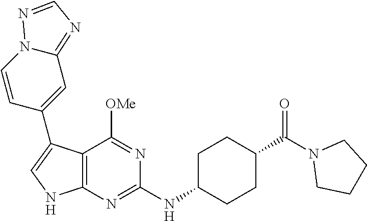 [SMILES: COc1nc(N[C@H]2CC[C@@H](C(=O)N3CCCC3)CC2)nc2[nH]cc(-c3ccn4ncnc4c3)c12] | 3380 |
| 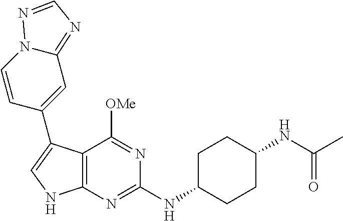 [SMILES: COc1nc(N[C@H]2CC[C@@H](NC(C)=O)CC2)nc2[nH]cc(-c3ccn4ncnc4c3)c12] | 3381 |
| 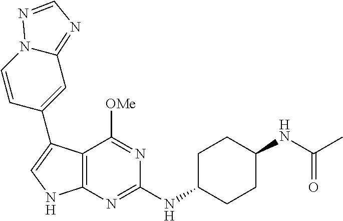 [SMILES: COc1nc(N[C@H]2CC[C@H](NC(C)=O)CC2)nc2[nH]cc(-c3ccn4ncnc4c3)c12] | 3382 |
| 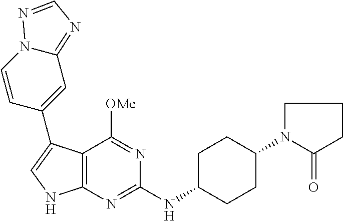 [SMILES: COc1nc(N[C@H]2CC[C@@H](N3CCCC3=O)CC2)nc2[nH]cc(-c3ccn4ncnc4c3)c12] | 3383 |
| 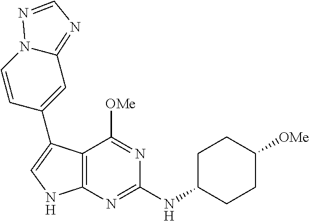 [SMILES: COc1nc(N[C@H]2CC[C@@H](OC)CC2)nc2[nH]cc(-c3ccn4ncnc4c3)c12] | 3384 |
| 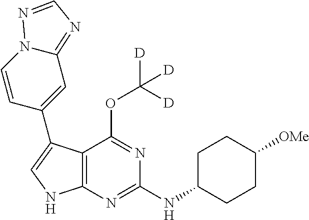 [SMILES: [2H]C([2H])([2H])Oc1nc(N[C@H]2CC[C@@H](OC)CC2)nc2[nH]cc(-c3ccn4ncnc4c3)c12] | 3385 |
| 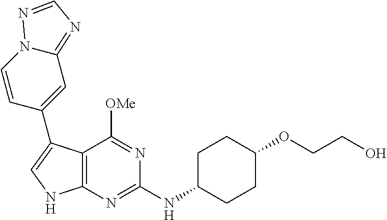 [SMILES: COc1nc(N[C@H]2CC[C@@H](OCCO)CC2)nc2[nH]cc(-c3ccn4ncnc4c3)c12] | 3386 |
| 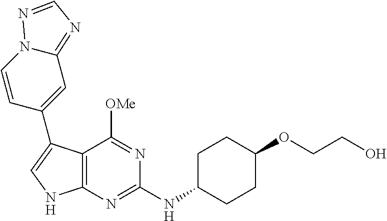 [SMILES: COc1nc(N[C@H]2CC[C@H](OCCO)CC2)nc2[nH]cc(-c3ccn4ncnc4c3)c12] | 3387 |
| 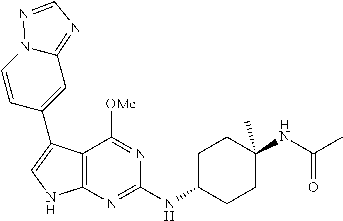 [SMILES: COc1nc(N[C@H]2CC[C@](C)(NC(C)=O)CC2)nc2[nH]cc(-c3ccn4ncnc4c3)c12] | 3388 |
| 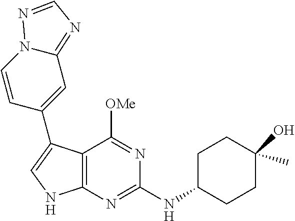 [SMILES: COc1nc(N[C@H]2CC[C@](C)(O)CC2)nc2[nH]cc(-c3ccn4ncnc4c3)c12] | 3389 |
| 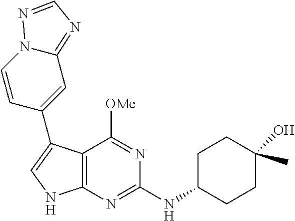 [SMILES: COc1nc(N[C@H]2CC[C@@](C)(O)CC2)nc2[nH]cc(-c3ccn4ncnc4c3)c12] | 3390 |
| 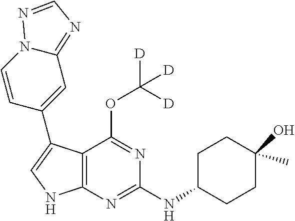 [SMILES: [2H]C([2H])([2H])Oc1nc(N[C@H]2CC[C@](C)(O)CC2)nc2[nH]cc(-c3ccn4ncnc4c3)c12] | 3391 |
| 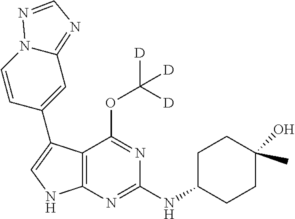 [SMILES: [2H]C([2H])([2H])Oc1nc(N[C@H]2CC[C@@](C)(O)CC2)nc2[nH]cc(-c3ccn4ncnc4c3)c12] | 3392 |
| 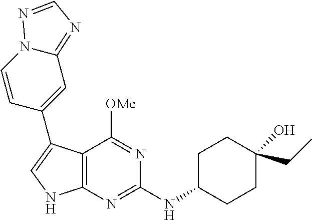 [SMILES: CC[C@]1(O)CC[C@@H](Nc2nc(OC)c3c(-c4ccn5ncnc5c4)c[nH]c3n2)CC1] | 3393 |
| 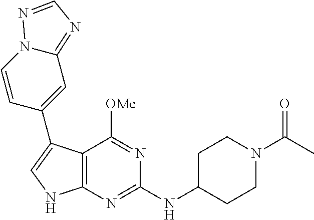 [SMILES: COc1nc(NC2CCN(C(C)=O)CC2)nc2[nH]cc(-c3ccn4ncnc4c3)c12] | 3394 |
| 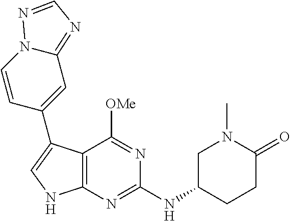 [SMILES: COc1nc(N[C@H]2CCC(=O)N(C)C2)nc2[nH]cc(-c3ccn4ncnc4c3)c12] | 3395 |
| 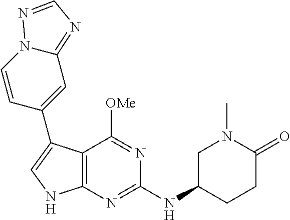 [SMILES: COc1nc(N[C@@H]2CCC(=O)N(C)C2)nc2[nH]cc(-c3ccn4ncnc4c3)c12] | 3396 |
| 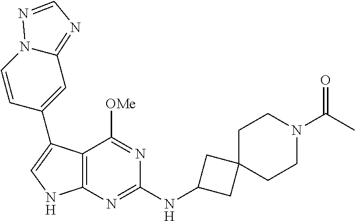 [SMILES: COc1nc(NC2CC3(CCN(C(C)=O)CC3)C2)nc2[nH]cc(-c3ccn4ncnc4c3)c12] | 3397 |
| 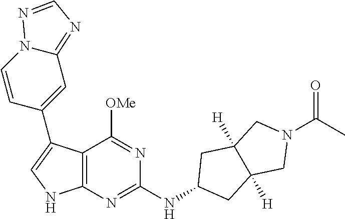 [SMILES: COc1nc(N[C@H]2C[C@@H]3CN(C(C)=O)C[C@@H]3C2)nc2[nH]cc(-c3ccn4ncnc4c3)c12] | 3398 |
| 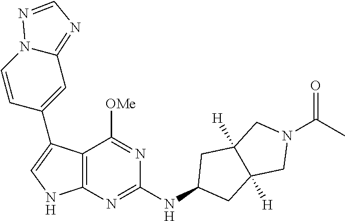 [SMILES: COc1nc(N[C@@H]2C[C@@H]3CN(C(C)=O)C[C@@H]3C2)nc2[nH]cc(-c3ccn4ncnc4c3)c12] | 3399 |
| 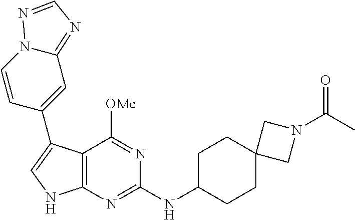 [SMILES: COc1nc(NC2CCC3(CC2)CN(C(C)=O)C3)nc2[nH]cc(-c3ccn4ncnc4c3)c12] | 3400 |
| 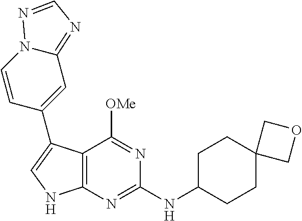 [SMILES: COc1nc(NC2CCC3(CC2)COC3)nc2[nH]cc(-c3ccn4ncnc4c3)c12] | 3401 |
| 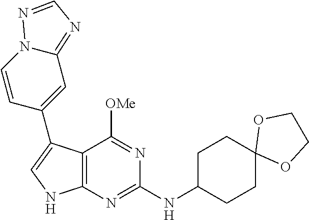 [SMILES: COc1nc(NC2CCC3(CC2)OCCO3)nc2[nH]cc(-c3ccn4ncnc4c3)c12] | 3402 |
| 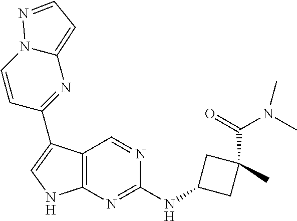 [SMILES: CN(C)C(=O)[C@]1(C)C[C@H](Nc2ncc3c(-c4ccn5nccc5n4)c[nH]c3n2)C1] | 3403 |
| 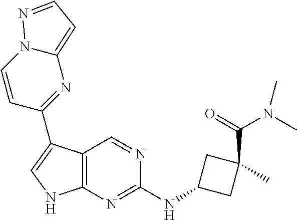 [SMILES: CN(C)C(=O)[C@]1(C)C[C@@H](Nc2ncc3c(-c4ccn5nccc5n4)c[nH]c3n2)C1] | 3404 |
| 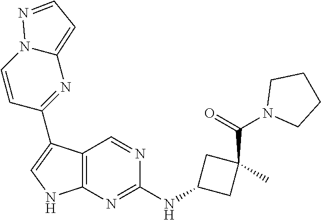 [SMILES: C[C@]1(C(=O)N2CCCC2)C[C@H](Nc2ncc3c(-c4ccn5nccc5n4)c[nH]c3n2)C1] | 3405 |
| 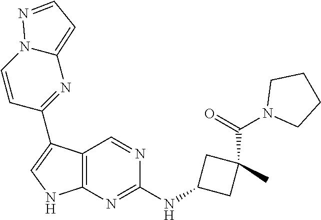 [SMILES: C[C@]1(C(=O)N2CCCC2)C[C@@H](Nc2ncc3c(-c4ccn5nccc5n4)c[nH]c3n2)C1] | 3406 |
| 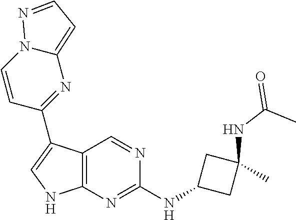 [SMILES: CC(=O)N[C@]1(C)C[C@@H](Nc2ncc3c(-c4ccn5nccc5n4)c[nH]c3n2)C1] | 3407 |
| 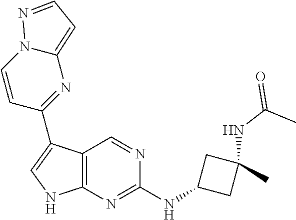 [SMILES: CC(=O)N[C@]1(C)C[C@H](Nc2ncc3c(-c4ccn5nccc5n4)c[nH]c3n2)C1] | 3408 |
| 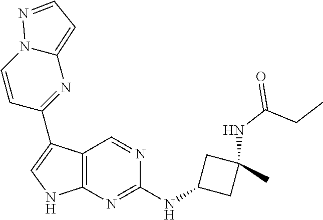 [SMILES: CCC(=O)N[C@]1(C)C[C@H](Nc2ncc3c(-c4ccn5nccc5n4)c[nH]c3n2)C1] | 3409 |
| 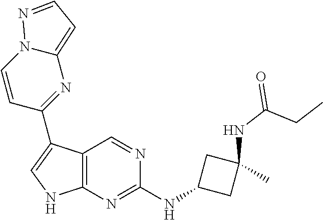 [SMILES: CCC(=O)N[C@]1(C)C[C@@H](Nc2ncc3c(-c4ccn5nccc5n4)c[nH]c3n2)C1] | 3410 |
| 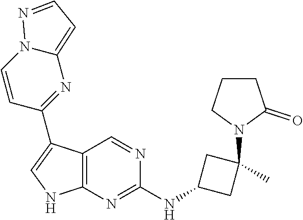 [SMILES: C[C@]1(N2CCCC2=O)C[C@H](Nc2ncc3c(-c4ccn5nccc5n4)c[nH]c3n2)C1] | 3411 |
| 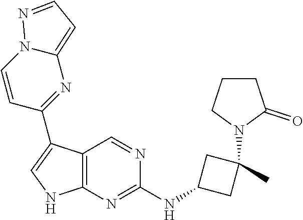 [SMILES: C[C@]1(N2CCCC2=O)C[C@@H](Nc2ncc3c(-c4ccn5nccc5n4)c[nH]c3n2)C1] | 3412 |
| 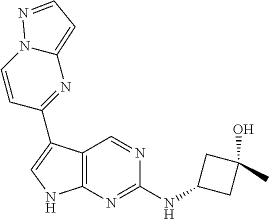 [SMILES: C[C@]1(O)C[C@@H](Nc2ncc3c(-c4ccn5nccc5n4)c[nH]c3n2)C1] | 3413 |
| 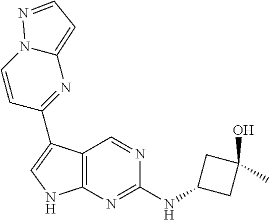 [SMILES: C[C@]1(O)C[C@H](Nc2ncc3c(-c4ccn5nccc5n4)c[nH]c3n2)C1] | 3414 |
| 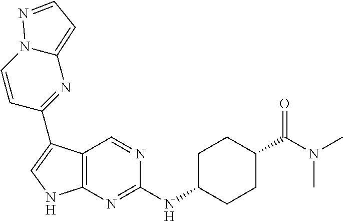 [SMILES: CN(C)C(=O)[C@H]1CC[C@@H](Nc2ncc3c(-c4ccn5nccc5n4)c[nH]c3n2)CC1] | 3415 |
| 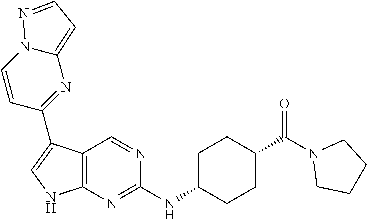 [SMILES: O=C([C@H]1CC[C@@H](Nc2ncc3c(-c4ccn5nccc5n4)c[nH]c3n2)CC1)N1CCCC1] | 3416 |
| 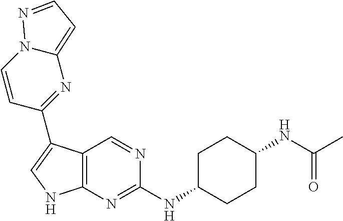 [SMILES: CC(=O)N[C@H]1CC[C@@H](Nc2ncc3c(-c4ccn5nccc5n4)c[nH]c3n2)CC1] | 3417 |
| 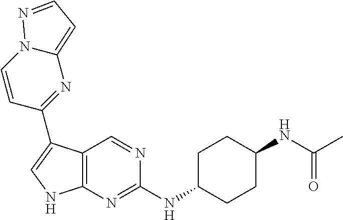 [SMILES: CC(=O)N[C@H]1CC[C@H](Nc2ncc3c(-c4ccn5nccc5n4)c[nH]c3n2)CC1] | 3418 |
| 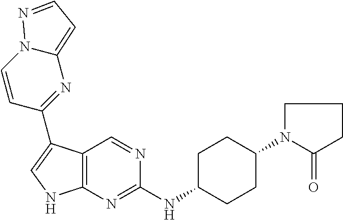 [SMILES: O=C1CCCN1[C@H]1CC[C@@H](Nc2ncc3c(-c4ccn5nccc5n4)c[nH]c3n2)CC1] | 3419 |
| 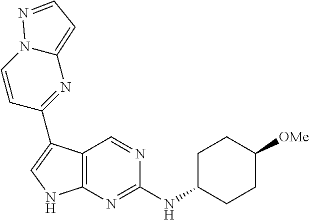 [SMILES: CO[C@H]1CC[C@H](Nc2ncc3c(-c4ccn5nccc5n4)c[nH]c3n2)CC1] | 3420 |
| 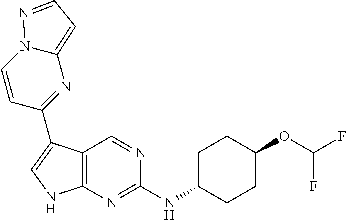 [SMILES: FC(F)O[C@H]1CC[C@H](Nc2ncc3c(-c4ccn5nccc5n4)c[nH]c3n2)CC1] | 3421 |
| 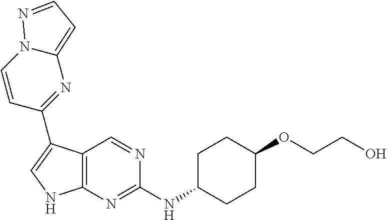 [SMILES: OCCO[C@H]1CC[C@H](Nc2ncc3c(-c4ccn5nccc5n4)c[nH]c3n2)CC1] | 3422 |
| 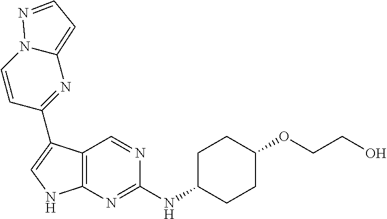 [SMILES: OCCO[C@H]1CC[C@@H](Nc2ncc3c(-c4ccn5nccc5n4)c[nH]c3n2)CC1] | 3423 |
| 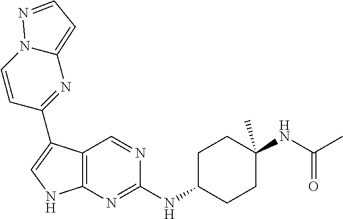 [SMILES: CC(=O)N[C@]1(C)CC[C@@H](Nc2ncc3c(-c4ccn5nccc5n4)c[nH]c3n2)CC1] | 3424 |
| 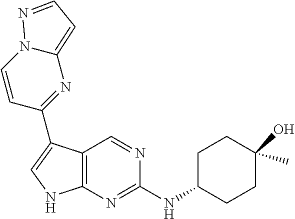 [SMILES: C[C@]1(O)CC[C@H](Nc2ncc3c(-c4ccn5nccc5n4)c[nH]c3n2)CC1] | 3425 |
| 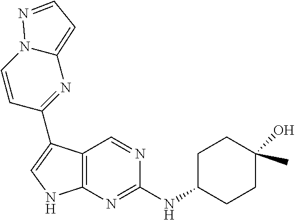 [SMILES: C[C@]1(O)CC[C@@H](Nc2ncc3c(-c4ccn5nccc5n4)c[nH]c3n2)CC1] | 3426 |
| 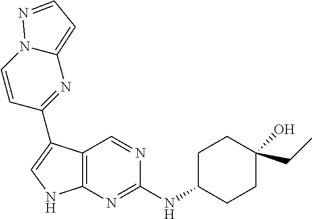 [SMILES: CC[C@]1(O)CC[C@@H](Nc2ncc3c(-c4ccn5nccc5n4)c[nH]c3n2)CC1] | 3427 |
| 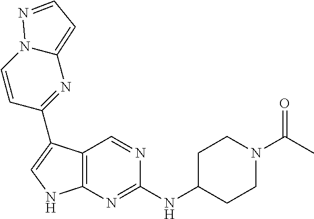 [SMILES: CC(=O)N1CCC(Nc2ncc3c(-c4ccn5nccc5n4)c[nH]c3n2)CC1] | 3428 |
| 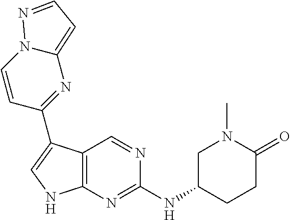 [SMILES: CN1C[C@@H](Nc2ncc3c(-c4ccn5nccc5n4)c[nH]c3n2)CCC1=O] | 3429 |
| 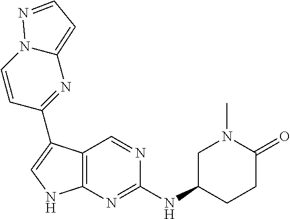 [SMILES: CN1C[C@H](Nc2ncc3c(-c4ccn5nccc5n4)c[nH]c3n2)CCC1=O] | 3430 |
| 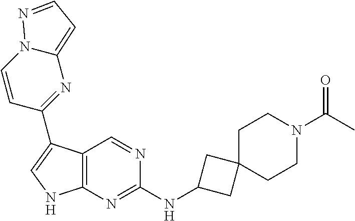 [SMILES: CC(=O)N1CCC2(CC1)CC(Nc1ncc3c(-c4ccn5nccc5n4)c[nH]c3n1)C2] | 3431 |
| 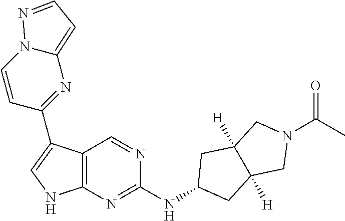 [SMILES: CC(=O)N1C[C@H]2C[C@H](Nc3ncc4c(-c5ccn6nccc6n5)c[nH]c4n3)C[C@H]2C1] | 3432 |
| 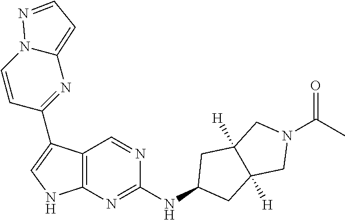 [SMILES: CC(=O)N1C[C@H]2C[C@@H](Nc3ncc4c(-c5ccn6nccc6n5)c[nH]c4n3)C[C@H]2C1] | 3433 |
| 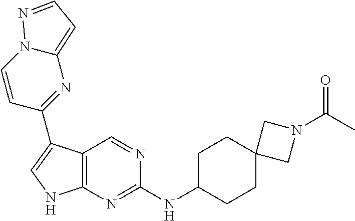 [SMILES: CC(=O)N1CC2(CCC(Nc3ncc4c(-c5ccn6nccc6n5)c[nH]c4n3)CC2)C1] | 3434 |
| 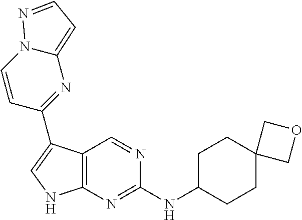 [SMILES: c1cc2nc(-c3c[nH]c4nc(NC5CCC6(CC5)COC6)ncc34)ccn2n1] | 3435 |
| 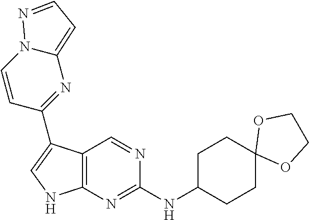 [SMILES: c1cc2nc(-c3c[nH]c4nc(NC5CCC6(CC5)OCCO6)ncc34)ccn2n1] | 3436 |
| 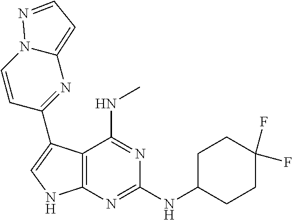 [SMILES: CNc1nc(NC2CCC(F)(F)CC2)nc2[nH]cc(-c3ccn4nccc4n3)c12] | 3437 |
| 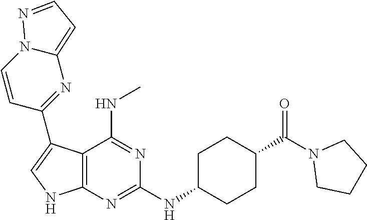 [SMILES: CNc1nc(N[C@H]2CC[C@@H](C(=O)N3CCCC3)CC2)nc2[nH]cc(-c3ccn4nccc4n3)c12] | 3438 |
| 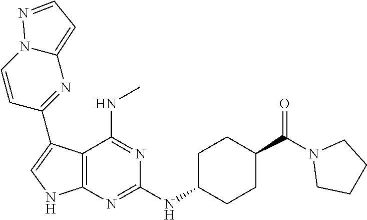 [SMILES: CNc1nc(N[C@H]2CC[C@H](C(=O)N3CCCC3)CC2)nc2[nH]cc(-c3ccn4nccc4n3)c12] | 3439 |
| 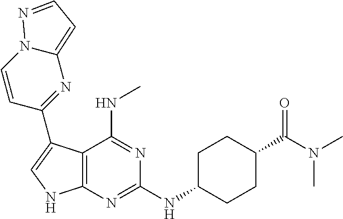 [SMILES: CNc1nc(N[C@H]2CC[C@@H](C(=O)N(C)C)CC2)nc2[nH]cc(-c3ccn4nccc4n3)c12] | 3440 |
| 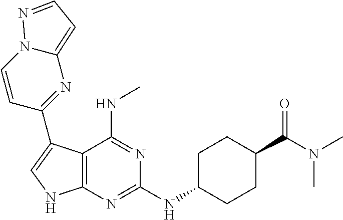 [SMILES: CNc1nc(N[C@H]2CC[C@H](C(=O)N(C)C)CC2)nc2[nH]cc(-c3ccn4nccc4n3)c12] | 3441 |
| 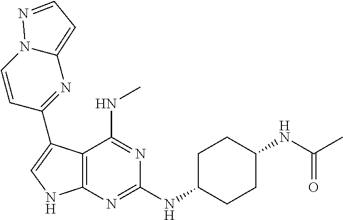 [SMILES: CNc1nc(N[C@H]2CC[C@@H](NC(C)=O)CC2)nc2[nH]cc(-c3ccn4nccc4n3)c12] | 3442 |
| 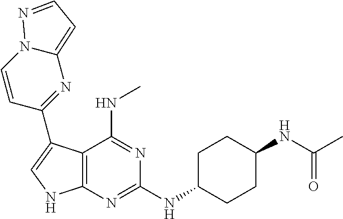 [SMILES: CNc1nc(N[C@H]2CC[C@H](NC(C)=O)CC2)nc2[nH]cc(-c3ccn4nccc4n3)c12] | 3443 |
| 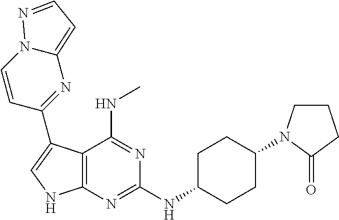 [SMILES: CNc1nc(N[C@H]2CC[C@@H](N3CCCC3=O)CC2)nc2[nH]cc(-c3ccn4nccc4n3)c12] | 3444 |
| 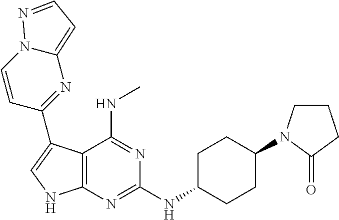 [SMILES: CNc1nc(N[C@H]2CC[C@H](N3CCCC3=O)CC2)nc2[nH]cc(-c3ccn4nccc4n3)c12] | 3445 |
| 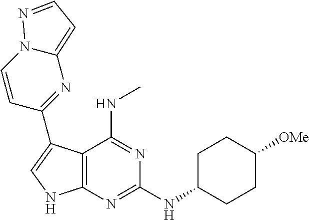 [SMILES: CNc1nc(N[C@H]2CC[C@@H](OC)CC2)nc2[nH]cc(-c3ccn4nccc4n3)c12] | 3446 |
| 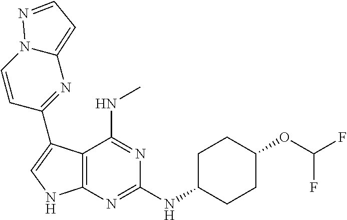 [SMILES: CNc1nc(N[C@H]2CC[C@@H](OC(F)F)CC2)nc2[nH]cc(-c3ccn4nccc4n3)c12] | 3447 |
| 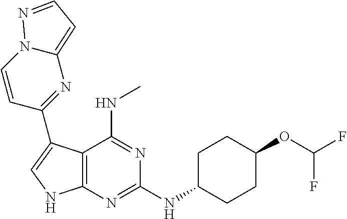 [SMILES: CNc1nc(N[C@H]2CC[C@H](OC(F)F)CC2)nc2[nH]cc(-c3ccn4nccc4n3)c12] | 3448 |
| 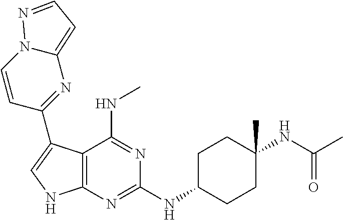 [SMILES: CNc1nc(N[C@H]2CC[C@@](C)(NC(C)=O)CC2)nc2[nH]cc(-c3ccn4nccc4n3)c12] | 3449 |
| 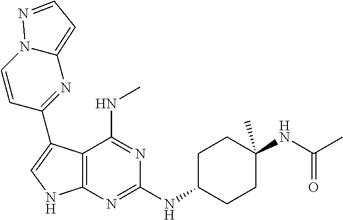 [SMILES: CNc1nc(N[C@H]2CC[C@](C)(NC(C)=O)CC2)nc2[nH]cc(-c3ccn4nccc4n3)c12] | 3450 |
| 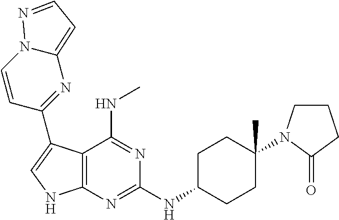 [SMILES: CNc1nc(N[C@H]2CC[C@@](C)(N3CCCC3=O)CC2)nc2[nH]cc(-c3ccn4nccc4n3)c12] | 3451 |
| 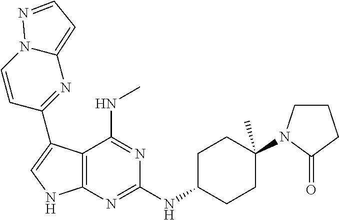 [SMILES: CNc1nc(N[C@H]2CC[C@](C)(N3CCCC3=O)CC2)nc2[nH]cc(-c3ccn4nccc4n3)c12] | 3452 |
| 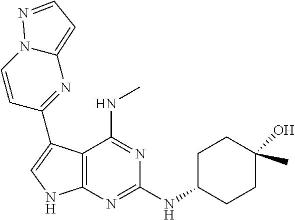 [SMILES: CNc1nc(N[C@H]2CC[C@@](C)(O)CC2)nc2[nH]cc(-c3ccn4nccc4n3)c12] | 3453 |
| 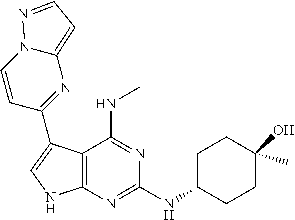 [SMILES: CNc1nc(N[C@H]2CC[C@](C)(O)CC2)nc2[nH]cc(-c3ccn4nccc4n3)c12] | 3454 |
| 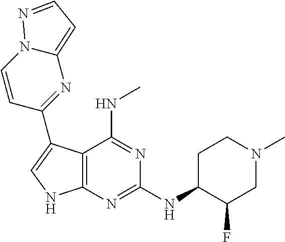 [SMILES: CNc1nc(N[C@H]2CCN(C)C[C@H]2F)nc2[nH]cc(-c3ccn4nccc4n3)c12] | 3455 |
| 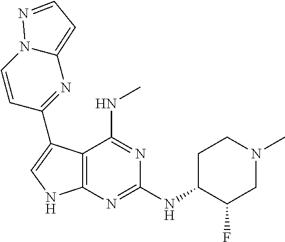 [SMILES: CNc1nc(N[C@@H]2CCN(C)C[C@@H]2F)nc2[nH]cc(-c3ccn4nccc4n3)c12] | 3456 |
| 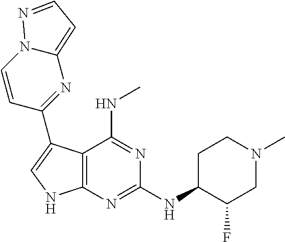 [SMILES: CNc1nc(N[C@H]2CCN(C)C[C@@H]2F)nc2[nH]cc(-c3ccn4nccc4n3)c12] | 3457 |
| 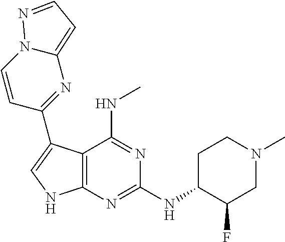 [SMILES: CNc1nc(N[C@@H]2CCN(C)C[C@H]2F)nc2[nH]cc(-c3ccn4nccc4n3)c12] | 3458 |
| 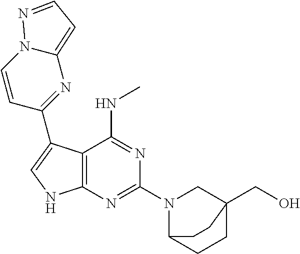 [SMILES: CNc1nc(N2CC3(CO)CCC2CC3)nc2[nH]cc(-c3ccn4nccc4n3)c12] | 3459 |
| 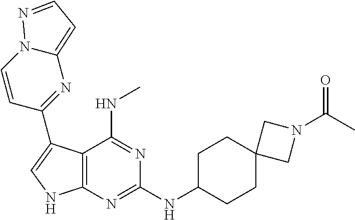 [SMILES: CNc1nc(NC2CCC3(CC2)CN(C(C)=O)C3)nc2[nH]cc(-c3ccn4nccc4n3)c12] | 3460 |
| 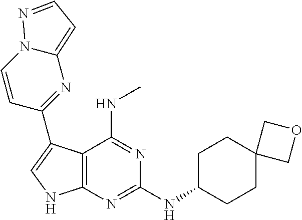 | 3461 |
| 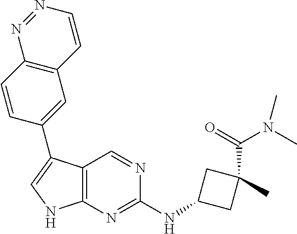 [SMILES: CN(C)C(=O)[C@]1(C)C[C@H](Nc2ncc3c(-c4ccc5nnccc5c4)c[nH]c3n2)C1] | 3462 |
| 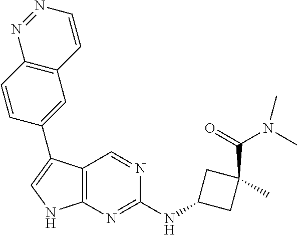 [SMILES: CN(C)C(=O)[C@]1(C)C[C@@H](Nc2ncc3c(-c4ccc5nnccc5c4)c[nH]c3n2)C1] | 3463 |
| 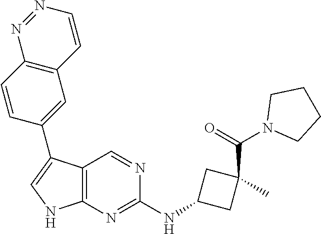 [SMILES: C[C@]1(C(=O)N2CCCC2)C[C@H](Nc2ncc3c(-c4ccc5nnccc5c4)c[nH]c3n2)C1] | 3464 |
| 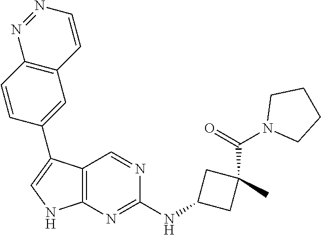 [SMILES: C[C@]1(C(=O)N2CCCC2)C[C@@H](Nc2ncc3c(-c4ccc5nnccc5c4)c[nH]c3n2)C1] | 3465 |
| 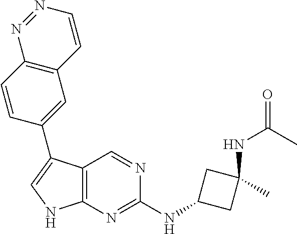 [SMILES: CC(=O)N[C@]1(C)C[C@@H](Nc2ncc3c(-c4ccc5nnccc5c4)c[nH]c3n2)C1] | 3466 |
| 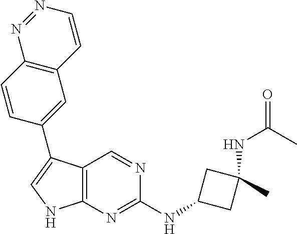 [SMILES: CC(=O)N[C@]1(C)C[C@H](Nc2ncc3c(-c4ccc5nnccc5c4)c[nH]c3n2)C1] | 3467 |
| 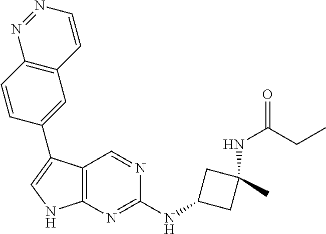 [SMILES: CCC(=O)N[C@]1(C)C[C@H](Nc2ncc3c(-c4ccc5nnccc5c4)c[nH]c3n2)C1] | 3468 |
| 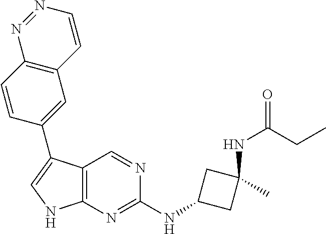 [SMILES: CCC(=O)N[C@]1(C)C[C@@H](Nc2ncc3c(-c4ccc5nnccc5c4)c[nH]c3n2)C1] | 3469 |
| 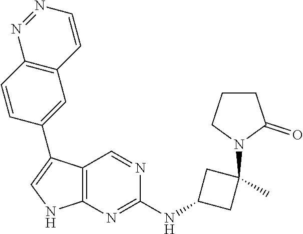 [SMILES: C[C@]1(N2CCCC2=O)C[C@H](Nc2ncc3c(-c4ccc5nnccc5c4)c[nH]c3n2)C1] | 3470 |
| 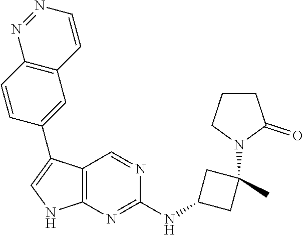 [SMILES: C[C@]1(N2CCCC2=O)C[C@@H](Nc2ncc3c(-c4ccc5nnccc5c4)c[nH]c3n2)C1] | 3471 |
| 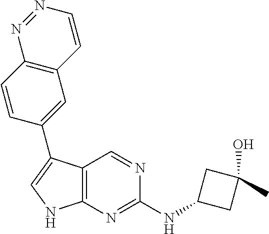 [SMILES: C[C@]1(O)C[C@@H](Nc2ncc3c(-c4ccc5nnccc5c4)c[nH]c3n2)C1] | 3472 |
| 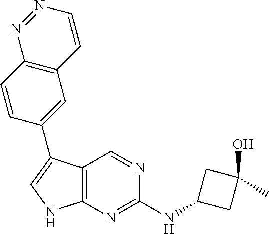 [SMILES: C[C@]1(O)C[C@H](Nc2ncc3c(-c4ccc5nnccc5c4)c[nH]c3n2)C1] | 3473 |
| 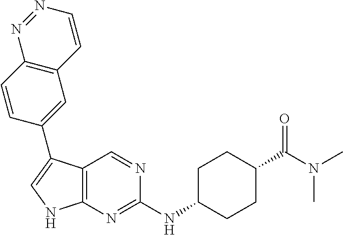 [SMILES: CN(C)C(=O)[C@H]1CC[C@@H](Nc2ncc3c(-c4ccc5nnccc5c4)c[nH]c3n2)CC1] | 3474 |
| 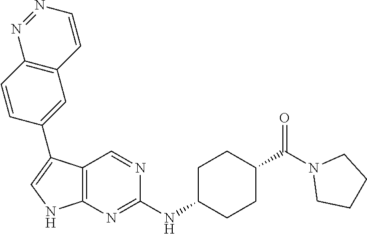 [SMILES: O=C([C@H]1CC[C@@H](Nc2ncc3c(-c4ccc5nnccc5c4)c[nH]c3n2)CC1)N1CCCC1] | 3475 |
| 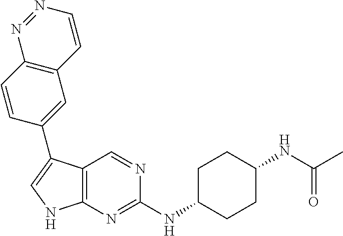 [SMILES: CC(=O)N[C@H]1CC[C@@H](Nc2ncc3c(-c4ccc5nnccc5c4)c[nH]c3n2)CC1] | 3476 |
| 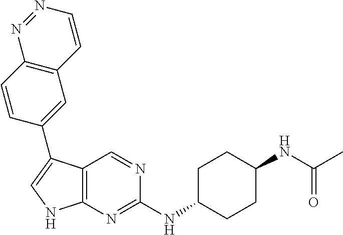 [SMILES: CC(=O)N[C@H]1CC[C@H](Nc2ncc3c(-c4ccc5nnccc5c4)c[nH]c3n2)CC1] | 3477 |
| 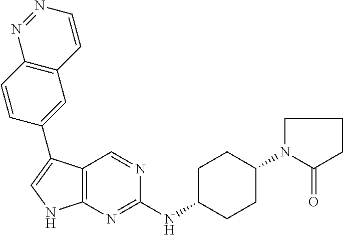 [SMILES: O=C1CCCN1[C@H]1CC[C@@H](Nc2ncc3c(-c4ccc5nnccc5c4)c[nH]c3n2)CC1] | 3478 |
| 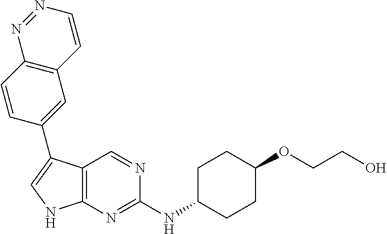 [SMILES: OCCO[C@H]1CC[C@H](Nc2ncc3c(-c4ccc5nnccc5c4)c[nH]c3n2)CC1] | 3479 |
| 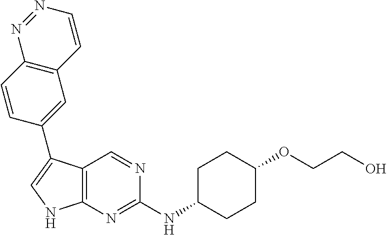 [SMILES: OCCO[C@H]1CC[C@@H](Nc2ncc3c(-c4ccc5nnccc5c4)c[nH]c3n2)CC1] | 3480 |
| 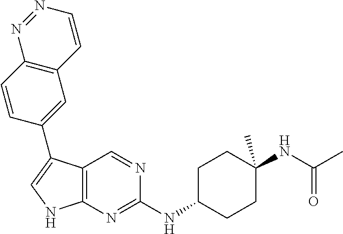 [SMILES: CC(=O)N[C@]1(C)CC[C@@H](Nc2ncc3c(-c4ccc5nnccc5c4)c[nH]c3n2)CC1] | 3481 |
| 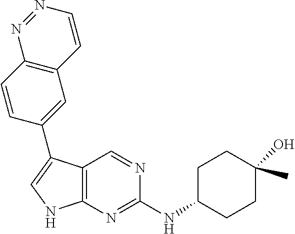 [SMILES: C[C@]1(O)CC[C@@H](Nc2ncc3c(-c4ccc5nnccc5c4)c[nH]c3n2)CC1] | 3482 |
| 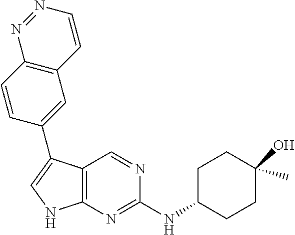 [SMILES: C[C@]1(O)CC[C@H](Nc2ncc3c(-c4ccc5nnccc5c4)c[nH]c3n2)CC1] | 3483 |
| 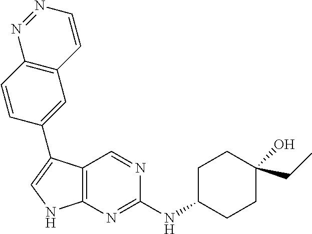 [SMILES: CC[C@]1(O)CC[C@@H](Nc2ncc3c(-c4ccc5nnccc5c4)c[nH]c3n2)CC1] | 3484 |
| 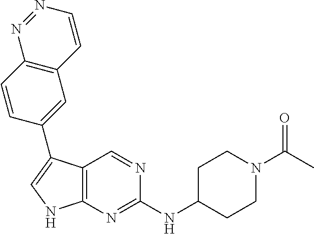 [SMILES: CC(=O)N1CCC(Nc2ncc3c(-c4ccc5nnccc5c4)c[nH]c3n2)CC1] | 3485 |
| 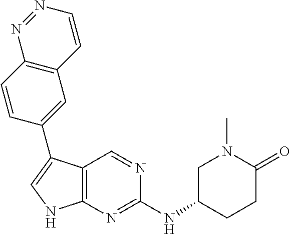 [SMILES: CN1C[C@@H](Nc2ncc3c(-c4ccc5nnccc5c4)c[nH]c3n2)CCC1=O] | 3486 |
| 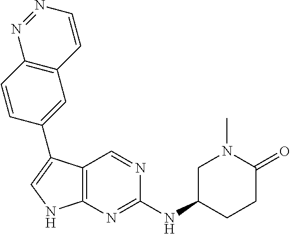 [SMILES: CN1C[C@H](Nc2ncc3c(-c4ccc5nnccc5c4)c[nH]c3n2)CCC1=O] | 3487 |
| 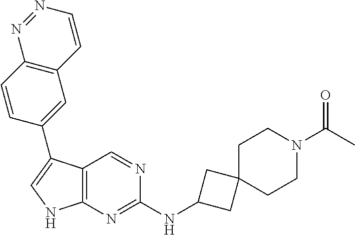 [SMILES: CC(=O)N1CCC2(CC1)CC(Nc1ncc3c(-c4ccc5nnccc5c4)c[nH]c3n1)C2] | 3488 |
| 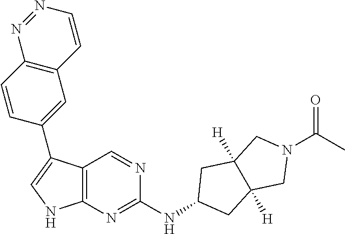 [SMILES: CC(=O)N1C[C@H]2C[C@H](Nc3ncc4c(-c5ccc6nnccc6c5)c[nH]c4n3)C[C@H]2C1] | 3489 |
| 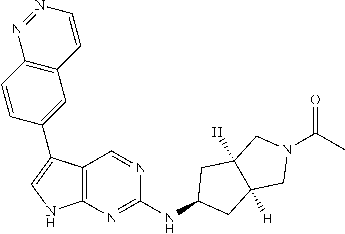 [SMILES: CC(=O)N1C[C@H]2C[C@@H](Nc3ncc4c(-c5ccc6nnccc6c5)c[nH]c4n3)C[C@H]2C1] | 3490 |
| 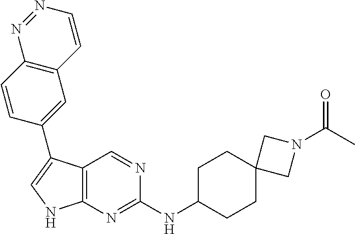 [SMILES: CC(=O)N1CC2(CCC(Nc3ncc4c(-c5ccc6nnccc6c5)c[nH]c4n3)CC2)C1] | 3491 |
| 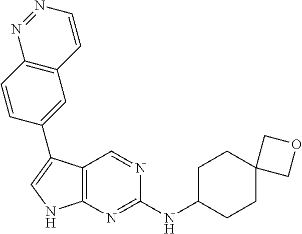 [SMILES: c1cc2cc(-c3c[nH]c4nc(NC5CCC6(CC5)COC6)ncc34)ccc2nn1] | 3492 |
| 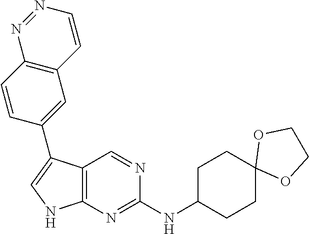 [SMILES: c1cc2cc(-c3c[nH]c4nc(NC5CCC6(CC5)OCCO6)ncc34)ccc2nn1] | 3493 |
| 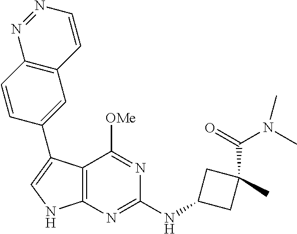 [SMILES: COc1nc(N[C@H]2C[C@@](C)(C(=O)N(C)C)C2)nc2[nH]cc(-c3ccc4nnccc4c3)c12] | 3494 |
| 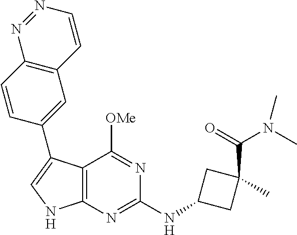 [SMILES: COc1nc(N[C@H]2C[C@](C)(C(=O)N(C)C)C2)nc2[nH]cc(-c3ccc4nnccc4c3)c12] | 3495 |
| 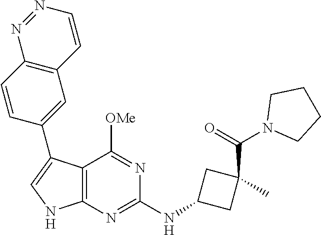 [SMILES: COc1nc(N[C@H]2C[C@](C)(C(=O)N3CCCC3)C2)nc2[nH]cc(-c3ccc4nnccc4c3)c12] | 3496 |
| 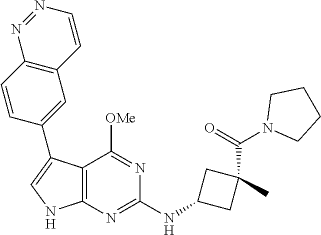 [SMILES: COc1nc(N[C@H]2C[C@@](C)(C(=O)N3CCCC3)C2)nc2[nH]cc(-c3ccc4nnccc4c3)c12] | 3497 |
| 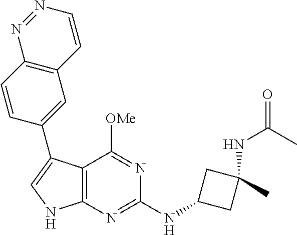 [SMILES: COc1nc(N[C@H]2C[C@@](C)(NC(C)=O)C2)nc2[nH]cc(-c3ccc4nnccc4c3)c12] | 3498 |
| 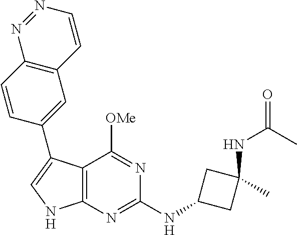 [SMILES: COc1nc(N[C@H]2C[C@](C)(NC(C)=O)C2)nc2[nH]cc(-c3ccc4nnccc4c3)c12] | 3499 |
| 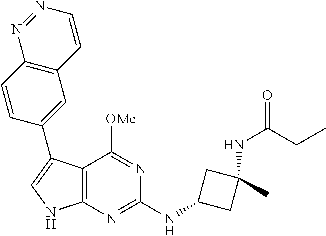 [SMILES: CCC(=O)N[C@]1(C)C[C@H](Nc2nc(OC)c3c(-c4ccc5nnccc5c4)c[nH]c3n2)C1] | 3500 |
| 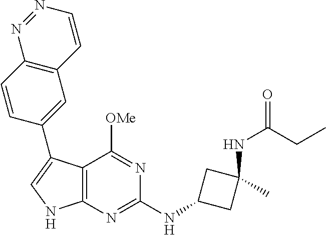 [SMILES: CCC(=O)N[C@]1(C)C[C@@H](Nc2nc(OC)c3c(-c4ccc5nnccc5c4)c[nH]c3n2)C1] | 3501 |
| 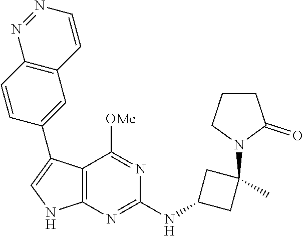 [SMILES: COc1nc(N[C@H]2C[C@](C)(N3CCCC3=O)C2)nc2[nH]cc(-c3ccc4nnccc4c3)c12] | 3502 |
| 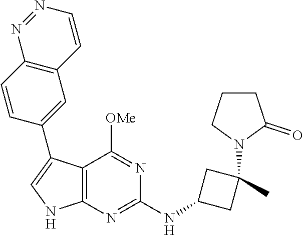 [SMILES: COc1nc(N[C@H]2C[C@@](C)(N3CCCC3=O)C2)nc2[nH]cc(-c3ccc4nnccc4c3)c12] | 3503 |
| 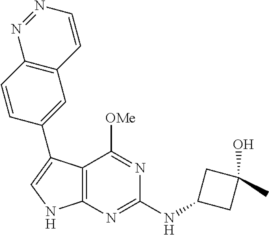 [SMILES: COc1nc(N[C@H]2C[C@@](C)(O)C2)nc2[nH]cc(-c3ccc4nnccc4c3)c12] | 3504 |
| 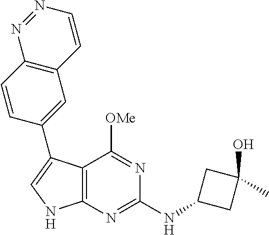 [SMILES: COc1nc(N[C@H]2C[C@](C)(O)C2)nc2[nH]cc(-c3ccc4nnccc4c3)c12] | 3505 |
| 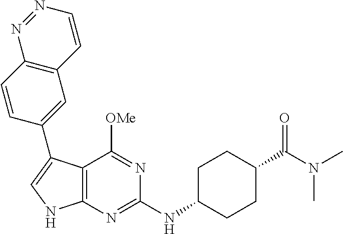 [SMILES: COc1nc(N[C@H]2CC[C@@H](C(=O)N(C)C)CC2)nc2[nH]cc(-c3ccc4nnccc4c3)c12] | 3506 |
| 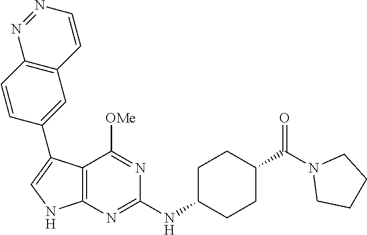 [SMILES: COc1nc(N[C@H]2CC[C@@H](C(=O)N3CCCC3)CC2)nc2[nH]cc(-c3ccc4nnccc4c3)c12] | 3507 |
| 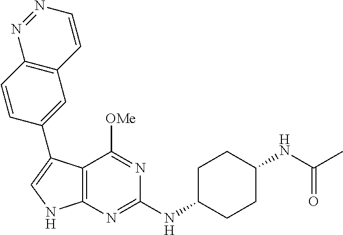 [SMILES: COc1nc(N[C@H]2CC[C@@H](NC(C)=O)CC2)nc2[nH]cc(-c3ccc4nnccc4c3)c12] | 3508 |
| 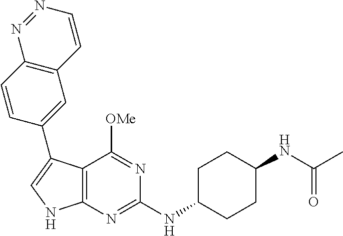 [SMILES: COc1nc(N[C@H]2CC[C@H](NC(C)=O)CC2)nc2[nH]cc(-c3ccc4nnccc4c3)c12] | 3509 |
| 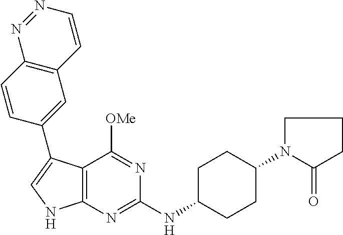 [SMILES: COc1nc(N[C@H]2CC[C@@H](N3CCCC3=O)CC2)nc2[nH]cc(-c3ccc4nnccc4c3)c12] | 3510 |
| 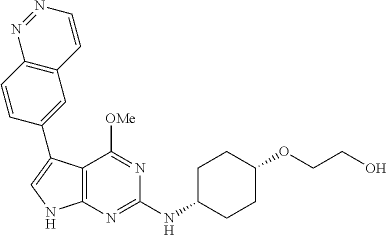 [SMILES: COc1nc(N[C@H]2CC[C@@H](OCCO)CC2)nc2[nH]cc(-c3ccc4nnccc4c3)c12] | 3511 |
| 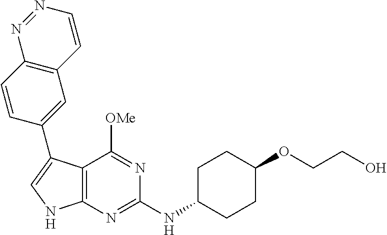 [SMILES: COc1nc(N[C@H]2CC[C@H](OCCO)CC2)nc2[nH]cc(-c3ccc4nnccc4c3)c12] | 3512 |
| 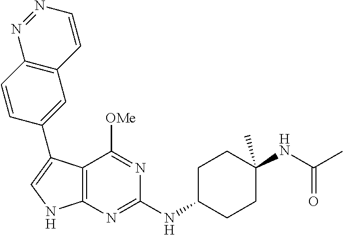 [SMILES: COc1nc(N[C@H]2CC[C@](C)(NC(C)=O)CC2)nc2[nH]cc(-c3ccc4nnccc4c3)c12] | 3513 |
| 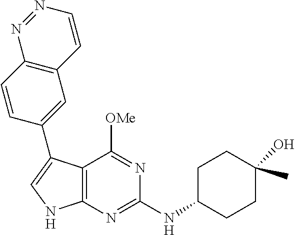 [SMILES: COc1nc(N[C@H]2CC[C@@](C)(O)CC2)nc2[nH]cc(-c3ccc4nnccc4c3)c12] | 3514 |
| 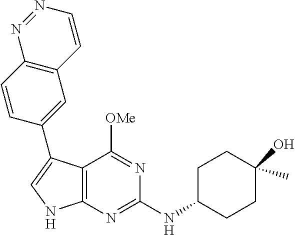 [SMILES: COc1nc(N[C@H]2CC[C@](C)(O)CC2)nc2[nH]cc(-c3ccc4nnccc4c3)c12] | 3515 |
| 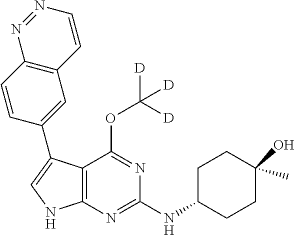 [SMILES: [2H]C([2H])([2H])Oc1nc(N[C@H]2CC[C@](C)(O)CC2)nc2[nH]cc(-c3ccc4nnccc4c3)c12] | 3516 |
| 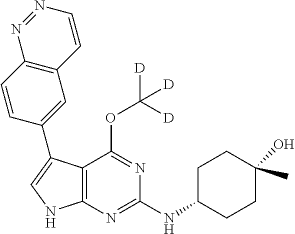 [SMILES: [2H]C([2H])([2H])Oc1nc(N[C@H]2CC[C@@](C)(O)CC2)nc2[nH]cc(-c3ccc4nnccc4c3)c12] | 3517 |
| 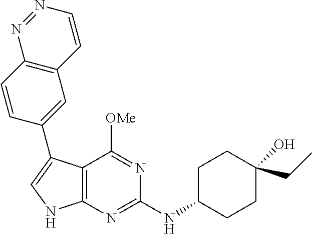 [SMILES: CC[C@]1(O)CC[C@@H](Nc2nc(OC)c3c(-c4ccc5nnccc5c4)c[nH]c3n2)CC1] | 3518 |
| 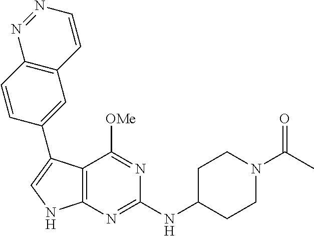 [SMILES: COc1nc(NC2CCN(C(C)=O)CC2)nc2[nH]cc(-c3ccc4nnccc4c3)c12] | 3519 |
| 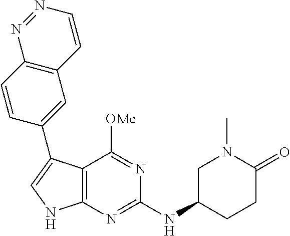 [SMILES: COc1nc(N[C@@H]2CCC(=O)N(C)C2)nc2[nH]cc(-c3ccc4nnccc4c3)c12] | 3520 |
| 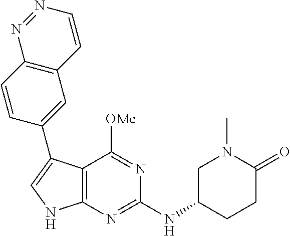 [SMILES: COc1nc(N[C@H]2CCC(=O)N(C)C2)nc2[nH]cc(-c3ccc4nnccc4c3)c12] | 3521 |
| 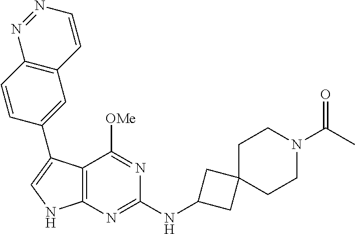 [SMILES: COc1nc(NC2CC3(CCN(C(C)=O)CC3)C2)nc2[nH]cc(-c3ccc4nnccc4c3)c12] | 3522 |
| 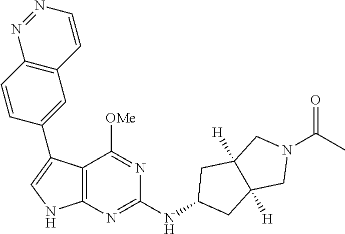 [SMILES: COc1nc(N[C@H]2C[C@@H]3CN(C(C)=O)C[C@@H]3C2)nc2[nH]cc(-c3ccc4nnccc4c3)c12] | 3523 |
| 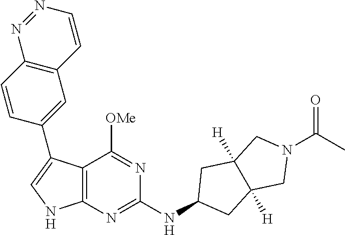 [SMILES: COc1nc(N[C@@H]2C[C@@H]3CN(C(C)=O)C[C@@H]3C2)nc2[nH]cc(-c3ccc4nnccc4c3)c12] | 3524 |
| 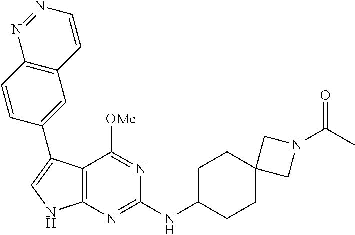 [SMILES: COc1nc(NC2CCC3(CC2)CN(C(C)=O)C3)nc2[nH]cc(-c3ccc4nnccc4c3)c12] | 3525 |
| 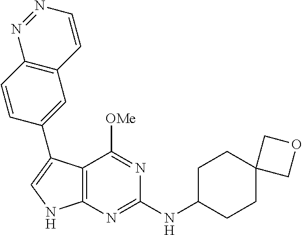 [SMILES: COc1nc(NC2CCC3(CC2)COC3)nc2[nH]cc(-c3ccc4nnccc4c3)c12] | 3526 |
| 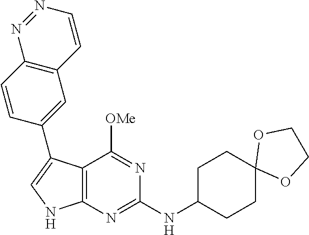 [SMILES: COc1nc(NC2CCC3(CC2)OCCO3)nc2[nH]cc(-c3ccc4nnccc4c3)c12] | 3527 |
| 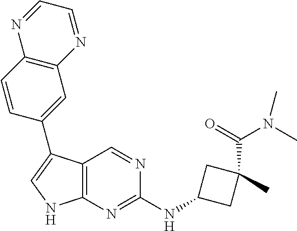 [SMILES: CN(C)C(=O)[C@]1(C)C[C@H](Nc2ncc3c(-c4ccc5nccnc5c4)c[nH]c3n2)C1] | 3528 |
| 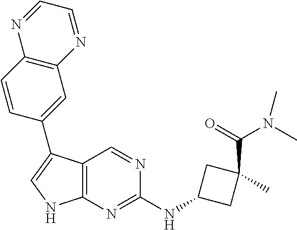 [SMILES: CN(C)C(=O)[C@]1(C)C[C@@H](Nc2ncc3c(-c4ccc5nccnc5c4)c[nH]c3n2)C1] | 3529 |
| 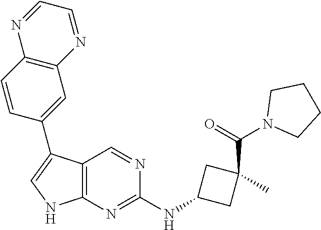 [SMILES: C[C@]1(C(=O)N2CCCC2)C[C@H](Nc2ncc3c(-c4ccc5nccnc5c4)c[nH]c3n2)C1] | 3530 |
| 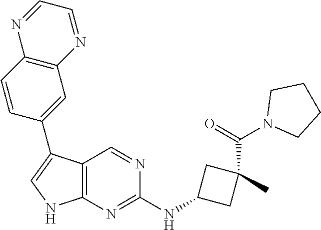 [SMILES: C[C@]1(C(=O)N2CCCC2)C[C@@H](Nc2ncc3c(-c4ccc5nccnc5c4)c[nH]c3n2)C1] | 3531 |
| 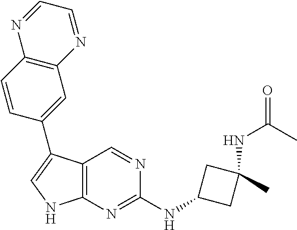 [SMILES: CC(=O)N[C@]1(C)C[C@H](Nc2ncc3c(-c4ccc5nccnc5c4)c[nH]c3n2)C1] | 3532 |
| 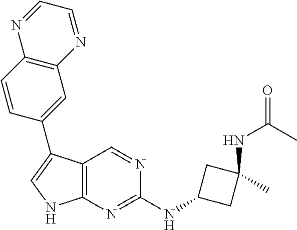 [SMILES: CC(=O)N[C@]1(C)C[C@@H](Nc2ncc3c(-c4ccc5nccnc5c4)c[nH]c3n2)C1] | 3533 |
| 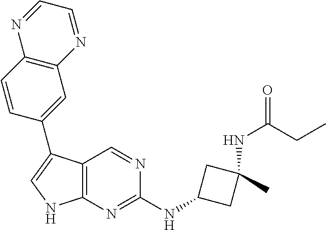 [SMILES: CCC(=O)N[C@]1(C)C[C@H](Nc2ncc3c(-c4ccc5nccnc5c4)c[nH]c3n2)C1] | 3534 |
| 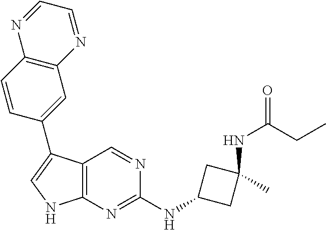 [SMILES: CCC(=O)N[C@]1(C)C[C@@H](Nc2ncc3c(-c4ccc5nccnc5c4)c[nH]c3n2)C1] | 3535 |
| 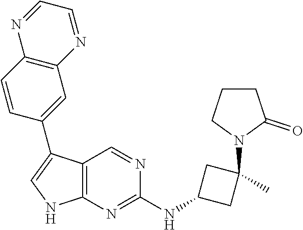 [SMILES: C[C@]1(N2CCCC2=O)C[C@H](Nc2ncc3c(-c4ccc5nccnc5c4)c[nH]c3n2)C1] | 3536 |
| 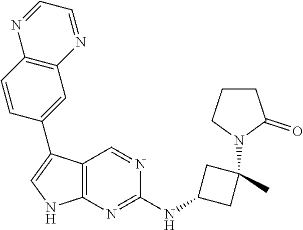 [SMILES: C[C@]1(N2CCCC2=O)C[C@@H](Nc2ncc3c(-c4ccc5nccnc5c4)c[nH]c3n2)C1] | 3537 |
| 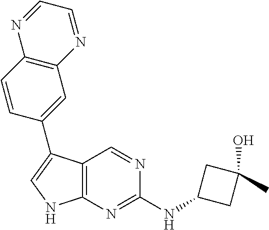 [SMILES: C[C@]1(O)C[C@@H](Nc2ncc3c(-c4ccc5nccnc5c4)c[nH]c3n2)C1] | 3538 |
| 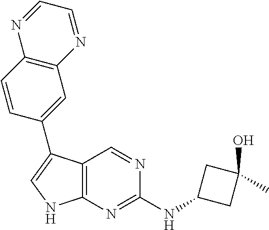 [SMILES: C[C@]1(O)C[C@H](Nc2ncc3c(-c4ccc5nccnc5c4)c[nH]c3n2)C1] | 3539 |
| 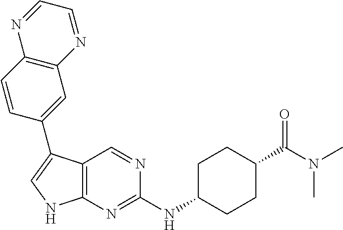 [SMILES: CN(C)C(=O)[C@H]1CC[C@@H](Nc2ncc3c(-c4ccc5nccnc5c4)c[nH]c3n2)CC1] | 3540 |
| 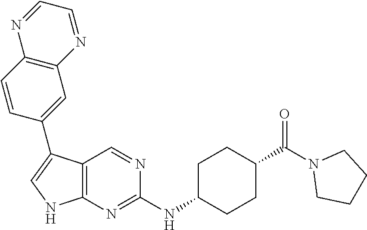 [SMILES: O=C([C@H]1CC[C@@H](Nc2ncc3c(-c4ccc5nccnc5c4)c[nH]c3n2)CC1)N1CCCC1] | 3541 |
| 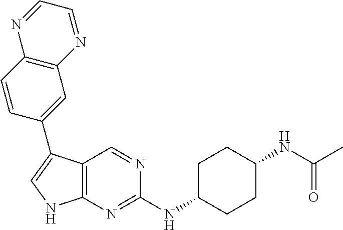 [SMILES: CC(=O)N[C@H]1CC[C@@H](Nc2ncc3c(-c4ccc5nccnc5c4)c[nH]c3n2)CC1] | 3542 |
| 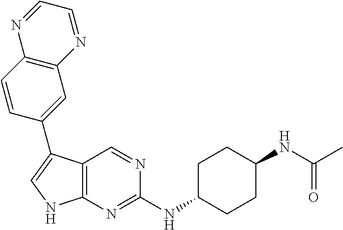 [SMILES: CC(=O)N[C@H]1CC[C@H](Nc2ncc3c(-c4ccc5nccnc5c4)c[nH]c3n2)CC1] | 3543 |
| 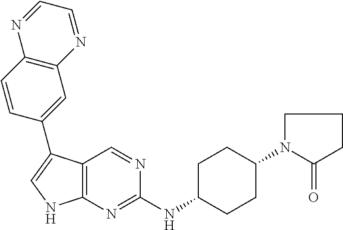 [SMILES: O=C1CCCN1[C@H]1CC[C@@H](Nc2ncc3c(-c4ccc5nccnc5c4)c[nH]c3n2)CC1] | 3544 |
| 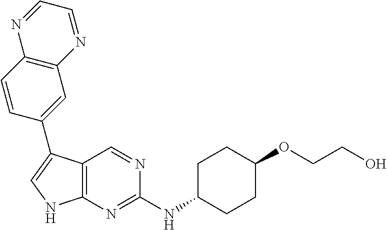 [SMILES: OCCO[C@H]1CC[C@H](Nc2ncc3c(-c4ccc5nccnc5c4)c[nH]c3n2)CC1] | 3545 |
| 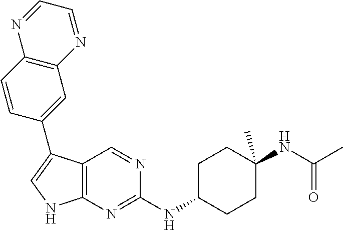 [SMILES: CC(=O)N[C@]1(C)CC[C@@H](Nc2ncc3c(-c4ccc5nccnc5c4)c[nH]c3n2)CC1] | 3546 |
| 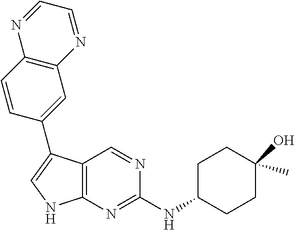 [SMILES: C[C@]1(O)CC[C@H](Nc2ncc3c(-c4ccc5nccnc5c4)c[nH]c3n2)CC1] | 3547 |
| 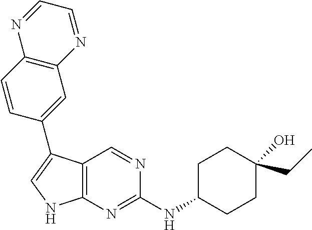 [SMILES: CC[C@]1(O)CC[C@@H](Nc2ncc3c(-c4ccc5nccnc5c4)c[nH]c3n2)CC1] | 3548 |
| 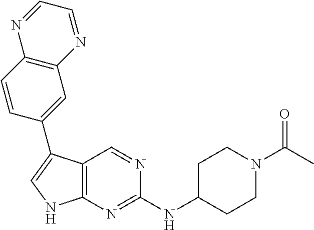 [SMILES: CC(=O)N1CCC(Nc2ncc3c(-c4ccc5nccnc5c4)c[nH]c3n2)CC1] | 3549 |
| 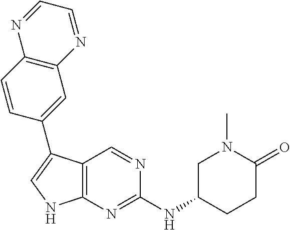 [SMILES: CN1C[C@@H](Nc2ncc3c(-c4ccc5nccnc5c4)c[nH]c3n2)CCC1=O] | 3550 |
| 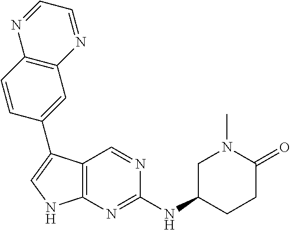 [SMILES: CN1C[C@H](Nc2ncc3c(-c4ccc5nccnc5c4)c[nH]c3n2)CCC1=O] | 3551 |
| 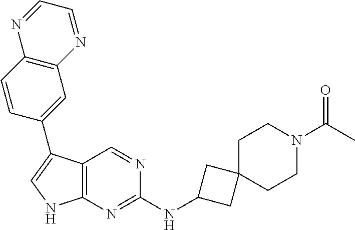 [SMILES: CC(=O)N1CCC2(CC1)CC(Nc1ncc3c(-c4ccc5nccnc5c4)c[nH]c3n1)C2] | 3552 |
| 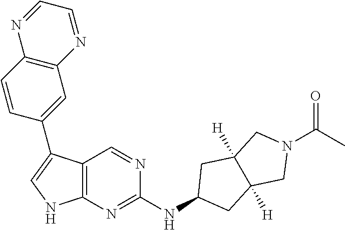 [SMILES: CC(=O)N1C[C@H]2C[C@@H](Nc3ncc4c(-c5ccc6nccnc6c5)c[nH]c4n3)C[C@H]2C1] | 3553 |
| 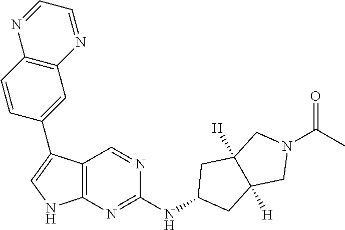 [SMILES: CC(=O)N1C[C@H]2C[C@H](Nc3ncc4c(-c5ccc6nccnc6c5)c[nH]c4n3)C[C@H]2C1] | 3554 |
| 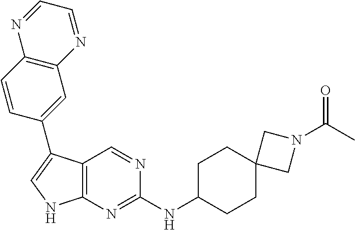 [SMILES: CC(=O)N1CC2(CCC(Nc3ncc4c(-c5ccc6nccnc6c5)c[nH]c4n3)CC2)C1] | 3555 |
| 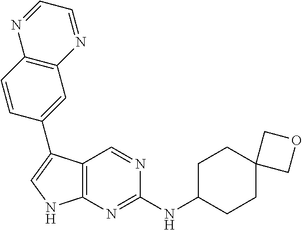 [SMILES: c1cnc2cc(-c3c[nH]c4nc(NC5CCC6(CC5)COC6)ncc34)ccc2n1] | 3556 |
| 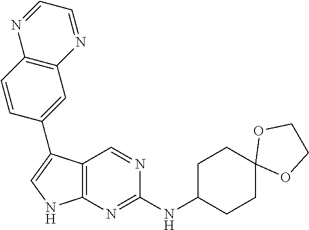 [SMILES: c1cnc2cc(-c3c[nH]c4nc(NC5CCC6(CC5)OCCO6)ncc34)ccc2n1] | 3557 |
| 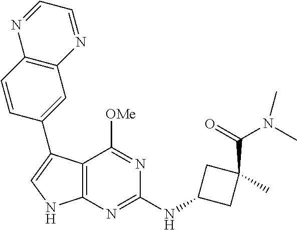 [SMILES: COc1nc(N[C@H]2C[C@](C)(C(=O)N(C)C)C2)nc2[nH]cc(-c3ccc4nccnc4c3)c12] | 3558 |
| 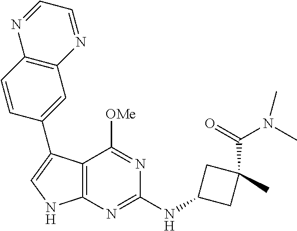 [SMILES: COc1nc(N[C@H]2C[C@@](C)(C(=O)N(C)C)C2)nc2[nH]cc(-c3ccc4nccnc4c3)c12] | 3559 |
| 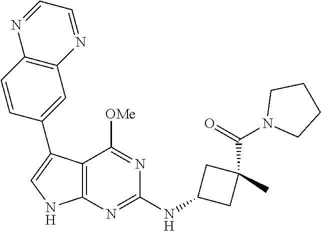 [SMILES: COc1nc(N[C@H]2C[C@@](C)(C(=O)N3CCCC3)C2)nc2[nH]cc(-c3ccc4nccnc4c3)c12] | 3560 |
| 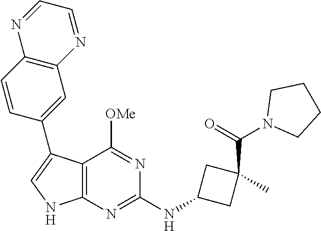 [SMILES: COc1nc(N[C@H]2C[C@](C)(C(=O)N3CCCC3)C2)nc2[nH]cc(-c3ccc4nccnc4c3)c12] | 3561 |
| 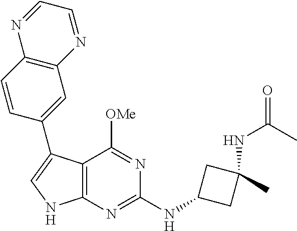 [SMILES: COc1nc(N[C@H]2C[C@@](C)(NC(C)=O)C2)nc2[nH]cc(-c3ccc4nccnc4c3)c12] | 3562 |
| 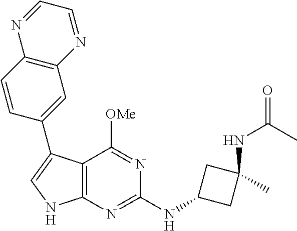 [SMILES: COc1nc(N[C@H]2C[C@](C)(NC(C)=O)C2)nc2[nH]cc(-c3ccc4nccnc4c3)c12] | 3563 |
| 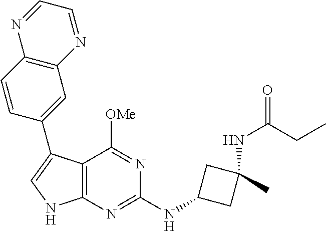 [SMILES: CCC(=O)N[C@]1(C)C[C@H](Nc2nc(OC)c3c(-c4ccc5nccnc5c4)c[nH]c3n2)C1] | 3564 |
| 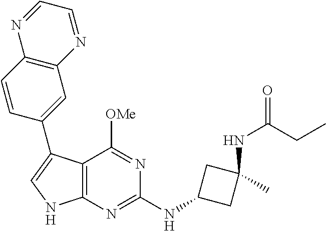 [SMILES: CCC(=O)N[C@]1(C)C[C@@H](Nc2nc(OC)c3c(-c4ccc5nccnc5c4)c[nH]c3n2)C1] | 3565 |
| 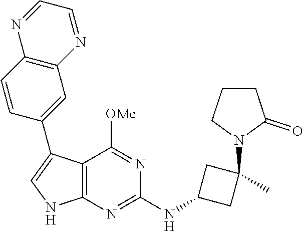 [SMILES: COc1nc(N[C@H]2C[C@](C)(N3CCCC3=O)C2)nc2[nH]cc(-c3ccc4nccnc4c3)c12] | 3566 |
| 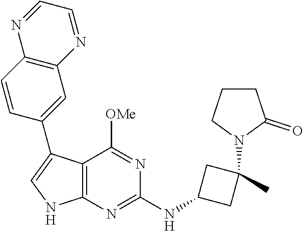 [SMILES: COc1nc(N[C@H]2C[C@@](C)(N3CCCC3=O)C2)nc2[nH]cc(-c3ccc4nccnc4c3)c12] | 3567 |
| 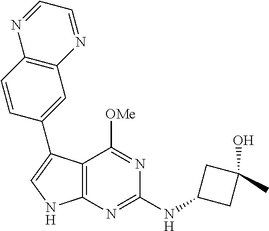 [SMILES: COc1nc(N[C@H]2C[C@@](C)(O)C2)nc2[nH]cc(-c3ccc4nccnc4c3)c12] | 3568 |
| 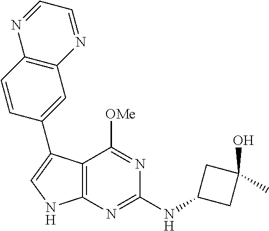 [SMILES: COc1nc(N[C@H]2C[C@](C)(O)C2)nc2[nH]cc(-c3ccc4nccnc4c3)c12] | 3569 |
| 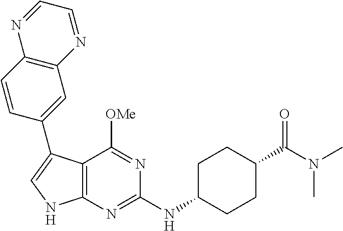 [SMILES: COc1nc(N[C@H]2CC[C@@H](C(=O)N(C)C)CC2)nc2[nH]cc(-c3ccc4nccnc4c3)c12] | 3570 |
| 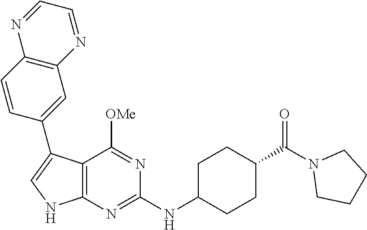 | 3571 |
| 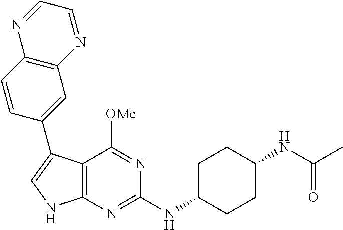 [SMILES: COc1nc(N[C@H]2CC[C@@H](NC(C)=O)CC2)nc2[nH]cc(-c3ccc4nccnc4c3)c12] | 3572 |
| 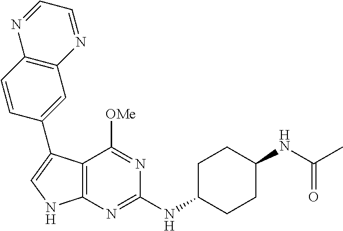 [SMILES: COc1nc(N[C@H]2CC[C@H](NC(C)=O)CC2)nc2[nH]cc(-c3ccc4nccnc4c3)c12] | 3573 |
| 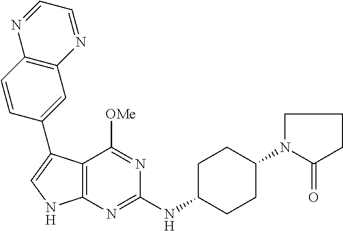 [SMILES: COc1nc(N[C@H]2CC[C@@H](N3CCCC3=O)CC2)nc2[nH]cc(-c3ccc4nccnc4c3)c12] | 3574 |
| 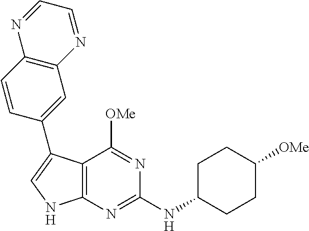 [SMILES: COc1nc(N[C@H]2CC[C@@H](OC)CC2)nc2[nH]cc(-c3ccc4nccnc4c3)c12] | 3575 |
| 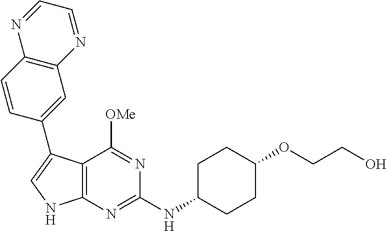 [SMILES: COc1nc(N[C@H]2CC[C@@H](OCCO)CC2)nc2[nH]cc(-c3ccc4nccnc4c3)c12] | 3576 |
| 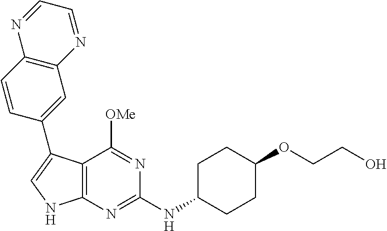 [SMILES: COc1nc(N[C@H]2CC[C@H](OCCO)CC2)nc2[nH]cc(-c3ccc4nccnc4c3)c12] | 3577 |
| 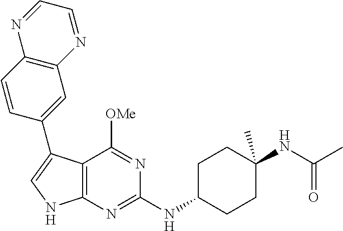 [SMILES: COc1nc(N[C@H]2CC[C@](C)(NC(C)=O)CC2)nc2[nH]cc(-c3ccc4nccnc4c3)c12] | 3578 |
| 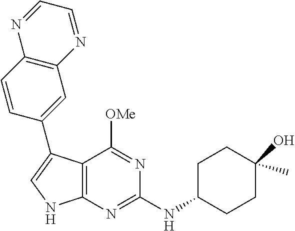 [SMILES: COc1nc(N[C@H]2CC[C@](C)(O)CC2)nc2[nH]cc(-c3ccc4nccnc4c3)c12] | 3579 |
| 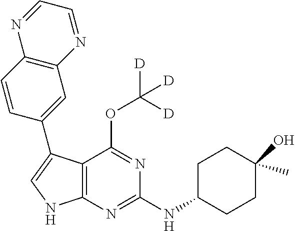 [SMILES: [2H]C([2H])([2H])Oc1nc(N[C@H]2CC[C@](C)(O)CC2)nc2[nH]cc(-c3ccc4nccnc4c3)c12] | 3580 |
| 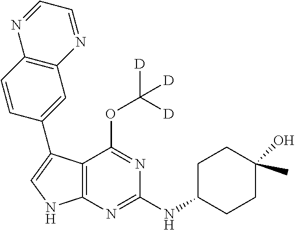 [SMILES: [2H]C([2H])([2H])Oc1nc(N[C@H]2CC[C@@](C)(O)CC2)nc2[nH]cc(-c3ccc4nccnc4c3)c12] | 3581 |
| 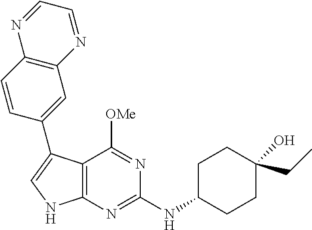 [SMILES: CC[C@]1(O)CC[C@@H](Nc2nc(OC)c3c(-c4ccc5nccnc5c4)c[nH]c3n2)CC1] | 3582 |
| 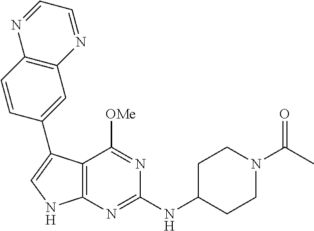 [SMILES: COc1nc(NC2CCN(C(C)=O)CC2)nc2[nH]cc(-c3ccc4nccnc4c3)c12] | 3583 |
| 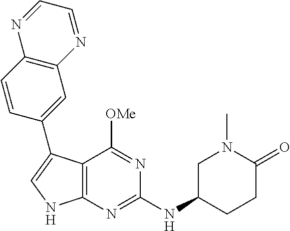 [SMILES: COc1nc(N[C@@H]2CCC(=O)N(C)C2)nc2[nH]cc(-c3ccc4nccnc4c3)c12] | 3584 |
| 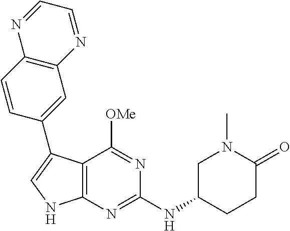 [SMILES: COc1nc(N[C@H]2CCC(=O)N(C)C2)nc2[nH]cc(-c3ccc4nccnc4c3)c12] | 3585 |
| 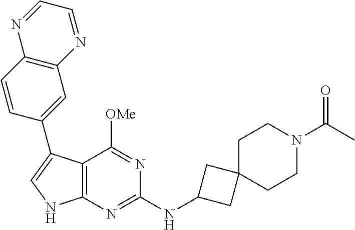 [SMILES: COc1nc(NC2CC3(CCN(C(C)=O)CC3)C2)nc2[nH]cc(-c3ccc4nccnc4c3)c12] | 3586 |
| 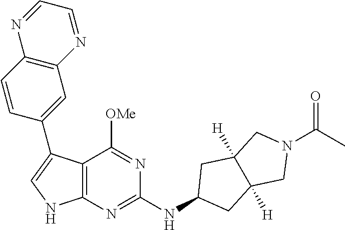 [SMILES: COc1nc(N[C@@H]2C[C@@H]3CN(C(C)=O)C[C@@H]3C2)nc2[nH]cc(-c3ccc4nccnc4c3)c12] | 3587 |
| 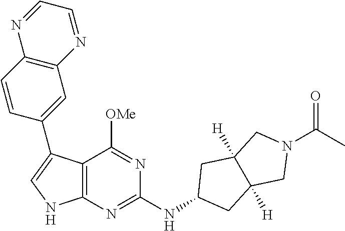 [SMILES: COc1nc(N[C@H]2C[C@@H]3CN(C(C)=O)C[C@@H]3C2)nc2[nH]cc(-c3ccc4nccnc4c3)c12] | 3588 |
| 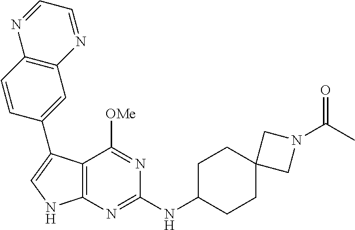 [SMILES: COc1nc(NC2CCC3(CC2)CN(C(C)=O)C3)nc2[nH]cc(-c3ccc4nccnc4c3)c12] | 3589 |
| 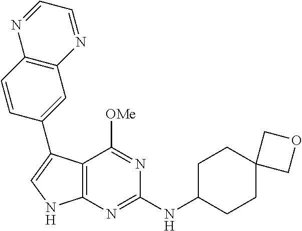 [SMILES: COc1nc(NC2CCC3(CC2)COC3)nc2[nH]cc(-c3ccc4nccnc4c3)c12] | 3590 |
| 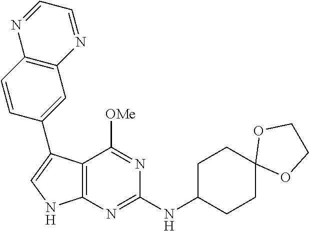 [SMILES: COc1nc(NC2CCC3(CC2)OCCO3)nc2[nH]cc(-c3ccc4nccnc4c3)c12] | 3591 |
| 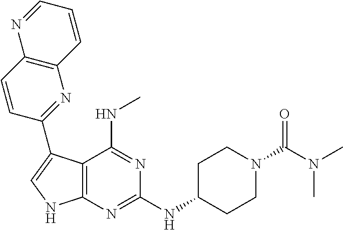 | 3592 |
| 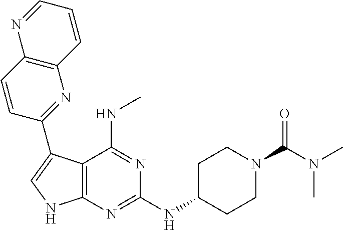 | 3593 |
| 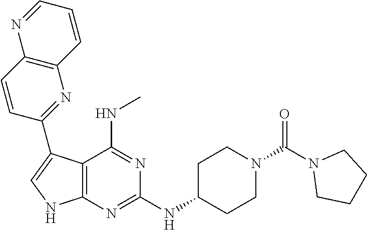 | 3594 |
| 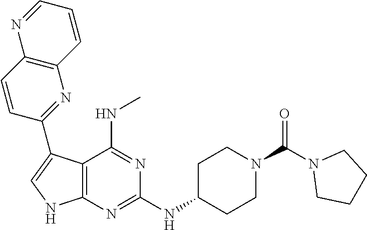 | 3595 |
| 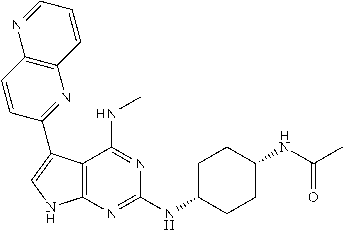 [SMILES: CNc1nc(N[C@H]2CC[C@@H](NC(C)=O)CC2)nc2[nH]cc(-c3ccc4ncccc4n3)c12] | 3596 |
| 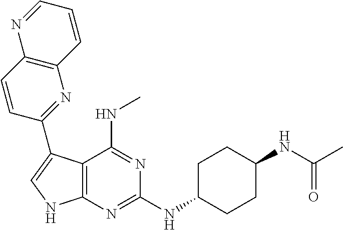 [SMILES: CNc1nc(N[C@H]2CC[C@H](NC(C)=O)CC2)nc2[nH]cc(-c3ccc4ncccc4n3)c12] | 3597 |
| 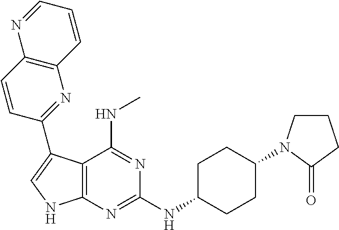 [SMILES: CNc1nc(N[C@H]2CC[C@@H](N3CCCC3=O)CC2)nc2[nH]cc(-c3ccc4ncccc4n3)c12] | 3598 |
| 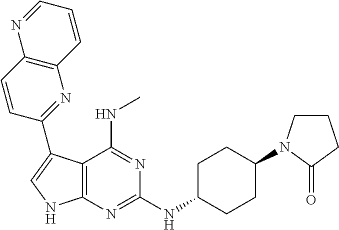 [SMILES: CNc1nc(N[C@H]2CC[C@H](N3CCCC3=O)CC2)nc2[nH]cc(-c3ccc4ncccc4n3)c12] | 3599 |
| 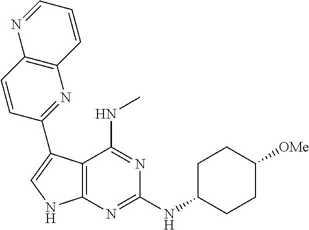 [SMILES: CNc1nc(N[C@H]2CC[C@@H](OC)CC2)nc2[nH]cc(-c3ccc4ncccc4n3)c12] | 3600 |
| 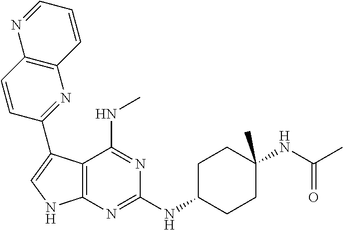 [SMILES: CNc1nc(N[C@H]2CC[C@@](C)(NC(C)=O)CC2)nc2[nH]cc(-c3ccc4ncccc4n3)c12] | 3601 |
| 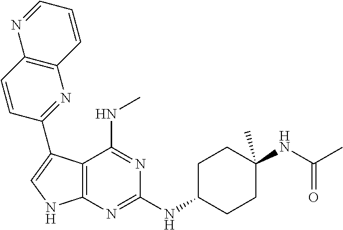 [SMILES: CNc1nc(N[C@H]2CC[C@](C)(NC(C)=O)CC2)nc2[nH]cc(-c3ccc4ncccc4n3)c12] | 3602 |
| 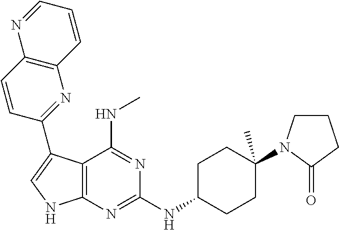 [SMILES: CNc1nc(N[C@H]2CC[C@](C)(N3CCCC3=O)CC2)nc2[nH]cc(-c3ccc4ncccc4n3)c12] | 3603 |
| 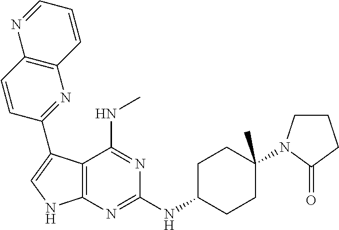 [SMILES: CNc1nc(N[C@H]2CC[C@@](C)(N3CCCC3=O)CC2)nc2[nH]cc(-c3ccc4ncccc4n3)c12] | 3604 |
| 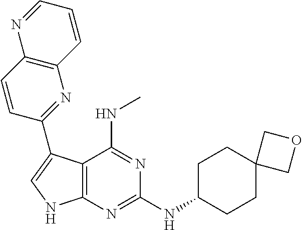 | 3605 |
| 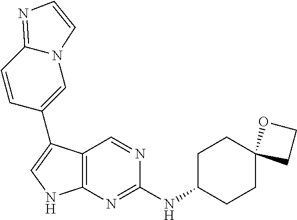 [SMILES: c1cn2cc(-c3c[nH]c4nc(N[C@H]5CC[C@]6(CCO6)CC5)ncc34)ccc2n1] | 3606 |
| 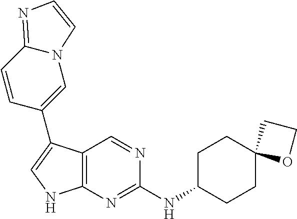 [SMILES: c1cn2cc(-c3c[nH]c4nc(N[C@H]5CC[C@@]6(CCO6)CC5)ncc34)ccc2n1] | 3607 |
| 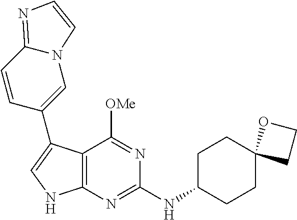 [SMILES: COc1nc(N[C@H]2CC[C@]3(CCO3)CC2)nc2[nH]cc(-c3ccc4nccn4c3)c12] | 3608 |
| 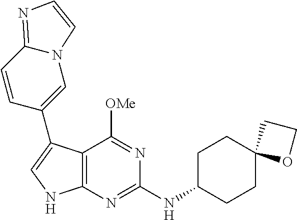 [SMILES: COc1nc(N[C@H]2CC[C@@]3(CCO3)CC2)nc2[nH]cc(-c3ccc4nccn4c3)c12] | 3609 |
| 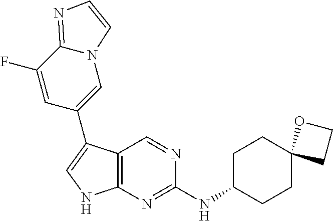 [SMILES: Fc1cc(-c2c[nH]c3nc(N[C@H]4CC[C@]5(CCO5)CC4)ncc23)cn2ccnc12] | 3610 |
| 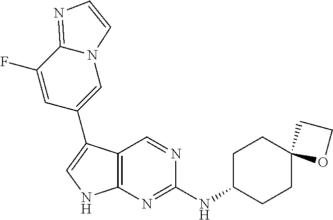 [SMILES: Fc1cc(-c2c[nH]c3nc(N[C@H]4CC[C@@]5(CCO5)CC4)ncc23)cn2ccnc12] | 3611 |
| 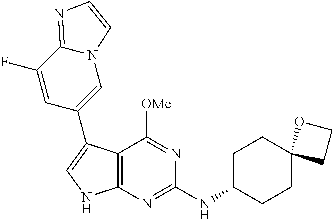 [SMILES: COc1nc(N[C@H]2CC[C@]3(CCO3)CC2)nc2[nH]cc(-c3cc(F)c4nccn4c3)c12] | 3612 |
| 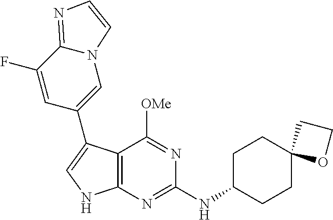 [SMILES: COc1nc(N[C@H]2CC[C@@]3(CCO3)CC2)nc2[nH]cc(-c3cc(F)c4nccn4c3)c12] | 3613 |
| 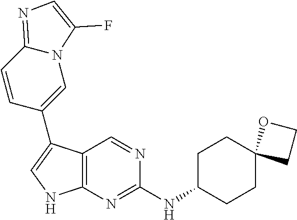 [SMILES: Fc1cnc2ccc(-c3c[nH]c4nc(N[C@H]5CC[C@]6(CCO6)CC5)ncc34)cn12] | 3614 |
| 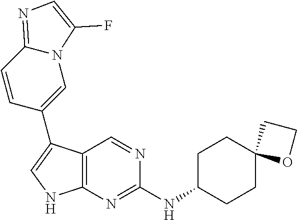 [SMILES: Fc1cnc2ccc(-c3c[nH]c4nc(N[C@H]5CC[C@@]6(CCO6)CC5)ncc34)cn12] | 3615 |
| 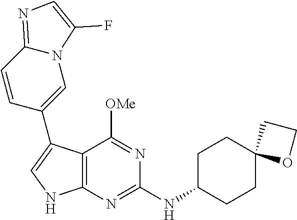 [SMILES: COc1nc(N[C@H]2CC[C@@]3(CCO3)CC2)nc2[nH]cc(-c3ccc4ncc(F)n4c3)c12] | 3616 |
| 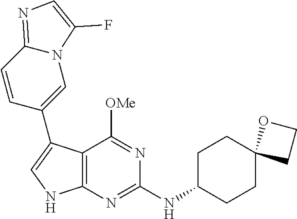 [SMILES: COc1nc(N[C@H]2CC[C@]3(CCO3)CC2)nc2[nH]cc(-c3ccc4ncc(F)n4c3)c12] | 3617 |
| 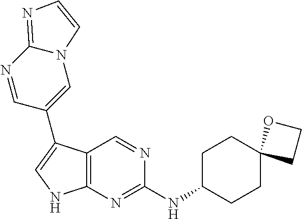 [SMILES: c1cn2cc(-c3c[nH]c4nc(N[C@H]5CC[C@]6(CCO6)CC5)ncc34)cnc2n1] | 3618 |
| 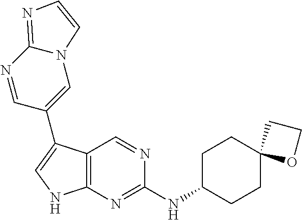 [SMILES: c1cn2cc(-c3c[nH]c4nc(N[C@H]5CC[C@@]6(CCO6)CC5)ncc34)cnc2n1] | 3619 |
| 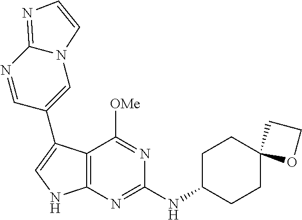 [SMILES: COc1nc(N[C@H]2CC[C@@]3(CCO3)CC2)nc2[nH]cc(-c3cnc4nccn4c3)c12] | 3620 |
| 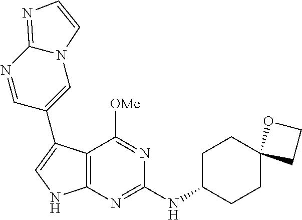 [SMILES: COc1nc(N[C@H]2CC[C@]3(CCO3)CC2)nc2[nH]cc(-c3cnc4nccn4c3)c12] | 3621 |
| 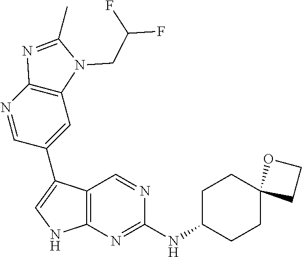 [SMILES: Cc1nc2ncc(-c3c[nH]c4nc(N[C@H]5CC[C@]6(CCO6)CC5)ncc34)cc2n1CC(F)F] | 3622 |
| 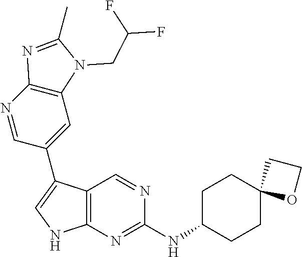 [SMILES: Cc1nc2ncc(-c3c[nH]c4nc(N[C@H]5CC[C@@]6(CCO6)CC5)ncc34)cc2n1CC(F)F] | 3623 |
| 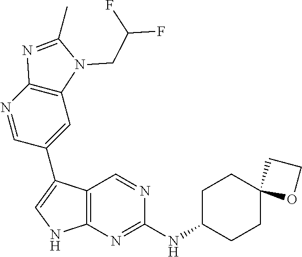 [SMILES: Cc1nc2ncc(-c3c[nH]c4nc(N[C@H]5CC[C@@]6(CCO6)CC5)ncc34)cc2n1CC(F)F] | 3624 |
| 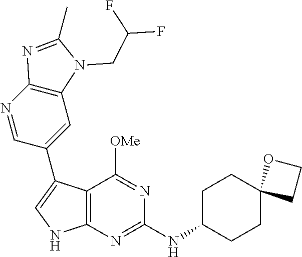 [SMILES: COc1nc(N[C@H]2CC[C@]3(CCO3)CC2)nc2[nH]cc(-c3cnc4nc(C)n(CC(F)F)c4c3)c12] | 3625 |
| 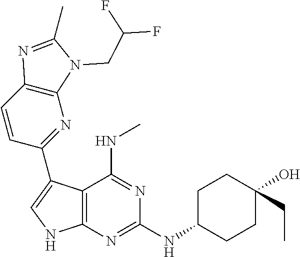 [SMILES: CC[C@]1(O)CC[C@@H](Nc2nc(NC)c3c(-c4ccc5nc(C)n(CC(F)F)c5n4)c[nH]c3n2)CC1] | 3626 |
| 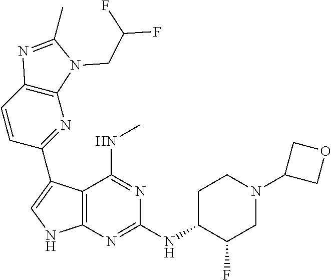 [SMILES: CNc1nc(N[C@@H]2CCN(C3COC3)C[C@@H]2F)nc2[nH]cc(-c3ccc4nc(C)n(CC(F)F)c4n3)c12] | 3627 |
| 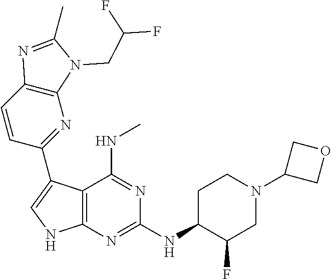 [SMILES: CNc1nc(N[C@H]2CCN(C3COC3)C[C@H]2F)nc2[nH]cc(-c3ccc4nc(C)n(CC(F)F)c4n3)c12] | 3628 |
| 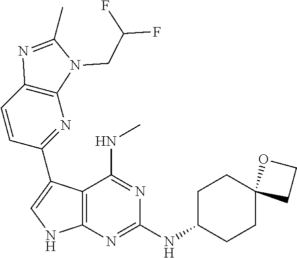 [SMILES: CNc1nc(N[C@H]2CC[C@]3(CCO3)CC2)nc2[nH]cc(-c3ccc4nc(C)n(CC(F)F)c4n3)c12] | 3629 |
| 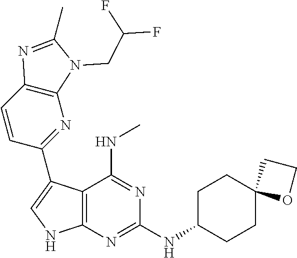 [SMILES: CNc1nc(N[C@H]2CC[C@@]3(CCO3)CC2)nc2[nH]cc(-c3ccc4nc(C)n(CC(F)F)c4n3)c12] | 3630 |
| 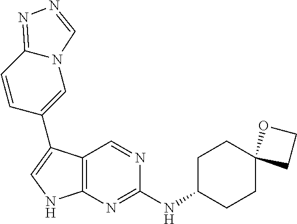 [SMILES: c1cc2nncn2cc1-c1c[nH]c2nc(N[C@H]3CC[C@]4(CCO4)CC3)ncc12] | 3631 |
| 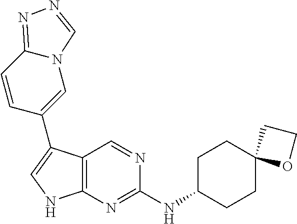 [SMILES: c1cc2nncn2cc1-c1c[nH]c2nc(N[C@H]3CC[C@@]4(CCO4)CC3)ncc12] | 3632 |
| 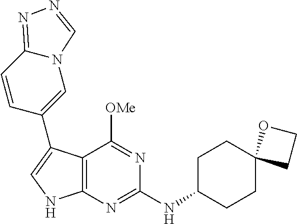 [SMILES: COc1nc(N[C@H]2CC[C@]3(CCO3)CC2)nc2[nH]cc(-c3ccc4nncn4c3)c12] | 3633 |
| 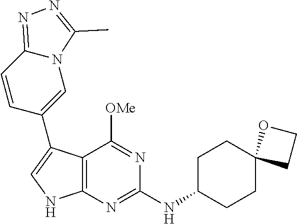 [SMILES: COc1nc(N[C@H]2CC[C@]3(CCO3)CC2)nc2[nH]cc(-c3ccc4nnc(C)n4c3)c12] | 3634 |
| 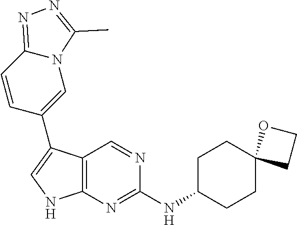 [SMILES: Cc1nnc2ccc(-c3c[nH]c4nc(N[C@H]5CC[C@]6(CCO6)CC5)ncc34)cn12] | 3635 |
| 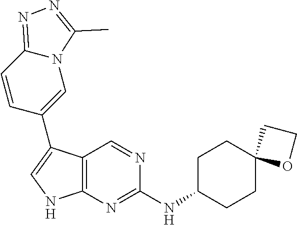 [SMILES: Cc1nnc2ccc(-c3c[nH]c4nc(N[C@H]5CC[C@@]6(CCO6)CC5)ncc34)cn12] | 3636 |
| 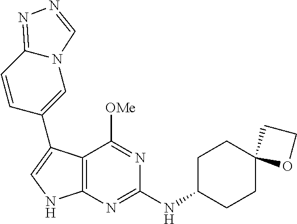 [SMILES: COc1nc(N[C@H]2CC[C@@]3(CCO3)CC2)nc2[nH]cc(-c3ccc4nncn4c3)c12] | 3637 |
| 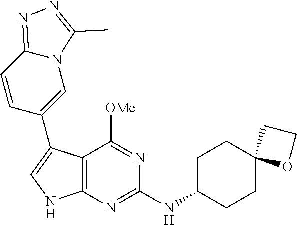 [SMILES: COc1nc(N[C@H]2CC[C@@]3(CCO3)CC2)nc2[nH]cc(-c3ccc4nnc(C)n4c3)c12] | 3638 |
| 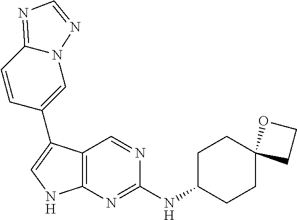 [SMILES: c1nc2ccc(-c3c[nH]c4nc(N[C@H]5CC[C@]6(CCO6)CC5)ncc34)cn2n1] | 3639 |
| 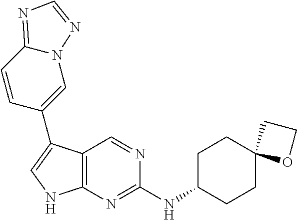 [SMILES: c1nc2ccc(-c3c[nH]c4nc(N[C@H]5CC[C@@]6(CCO6)CC5)ncc34)cn2n1] | 3640 |
| 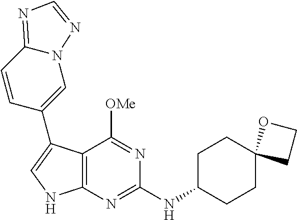 [SMILES: COc1nc(N[C@H]2CC[C@]3(CCO3)CC2)nc2[nH]cc(-c3ccc4ncnn4c3)c12] | 3641 |
| 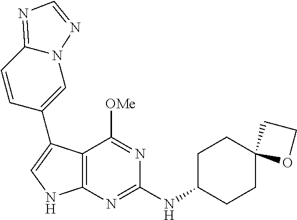 [SMILES: COc1nc(N[C@H]2CC[C@@]3(CCO3)CC2)nc2[nH]cc(-c3ccc4ncnn4c3)c12] | 3642 |
| 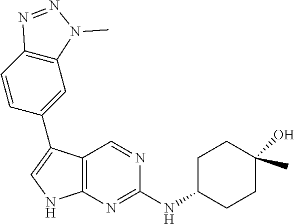 [SMILES: Cn1nnc2ccc(-c3c[nH]c4nc(N[C@H]5CC[C@@](C)(O)CC5)ncc34)cc21] | 3643 |
| 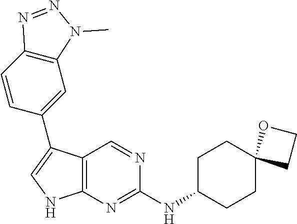 [SMILES: Cn1nnc2ccc(-c3c[nH]c4nc(N[C@H]5CC[C@]6(CCO6)CC5)ncc34)cc21] | 3644 |
| 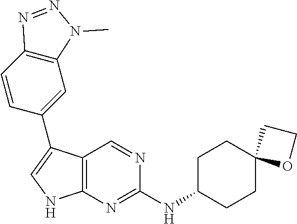 [SMILES: Cn1nnc2ccc(-c3c[nH]c4nc(N[C@H]5CC[C@@]6(CCO6)CC5)ncc34)cc21] | 3645 |
| 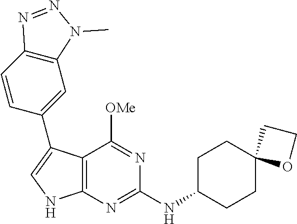 [SMILES: COc1nc(N[C@H]2CC[C@@]3(CCO3)CC2)nc2[nH]cc(-c3ccc4nnn(C)c4c3)c12] | 3646 |
| 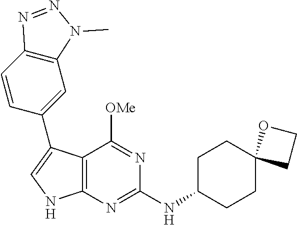 [SMILES: COc1nc(N[C@H]2CC[C@]3(CCO3)CC2)nc2[nH]cc(-c3ccc4nnn(C)c4c3)c12] | 3647 |
| 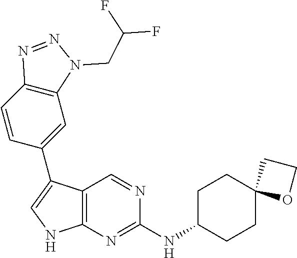 [SMILES: FC(F)Cn1nnc2ccc(-c3c[nH]c4nc(N[C@H]5CC[C@@]6(CCO6)CC5)ncc34)cc21] | 3648 |
| 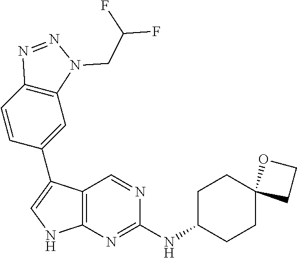 [SMILES: FC(F)Cn1nnc2ccc(-c3c[nH]c4nc(N[C@H]5CC[C@]6(CCO6)CC5)ncc34)cc21] | 3649 |
| 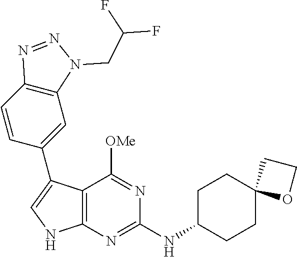 [SMILES: COc1nc(N[C@H]2CC[C@@]3(CCO3)CC2)nc2[nH]cc(-c3ccc4nnn(CC(F)F)c4c3)c12] | 3650 |
| 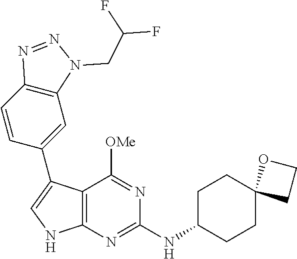 [SMILES: COc1nc(N[C@H]2CC[C@]3(CCO3)CC2)nc2[nH]cc(-c3ccc4nnn(CC(F)F)c4c3)c12] | 3651 |
| 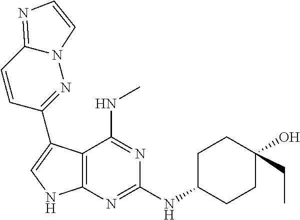 [SMILES: CC[C@]1(O)CC[C@@H](Nc2nc(NC)c3c(-c4ccc5nccn5n4)c[nH]c3n2)CC1] | 3652 |
| 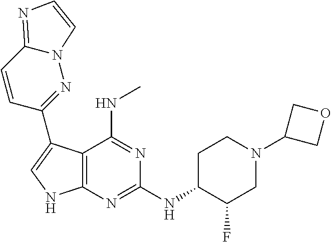 [SMILES: CNc1nc(N[C@@H]2CCN(C3COC3)C[C@@H]2F)nc2[nH]cc(-c3ccc4nccn4n3)c12] | 3653 |
| 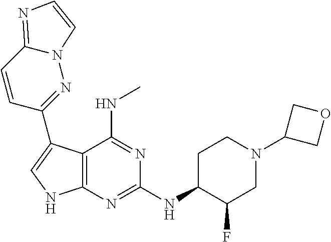 [SMILES: CNc1nc(N[C@H]2CCN(C3COC3)C[C@H]2F)nc2[nH]cc(-c3ccc4nccn4n3)c12] | 3654 |
| 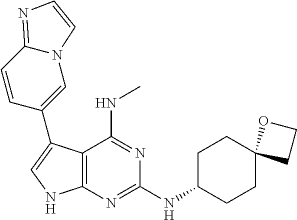 [SMILES: CNc1nc(N[C@H]2CC[C@]3(CCO3)CC2)nc2[nH]cc(-c3ccc4nccn4c3)c12] | 3655 |
| 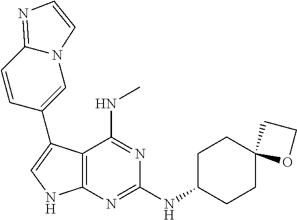 [SMILES: CNc1nc(N[C@H]2CC[C@@]3(CCO3)CC2)nc2[nH]cc(-c3ccc4nccn4c3)c12] | 3656 |
| 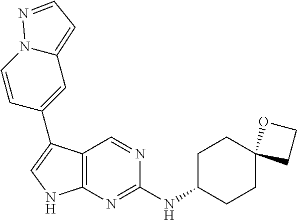 [SMILES: c1cc2cc(-c3c[nH]c4nc(N[C@H]5CC[C@]6(CCO6)CC5)ncc34)ccn2n1] | 3657 |
| 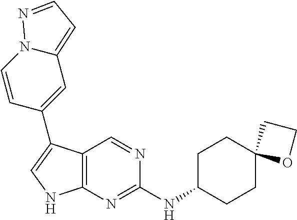 [SMILES: c1cc2cc(-c3c[nH]c4nc(N[C@H]5CC[C@@]6(CCO6)CC5)ncc34)ccn2n1] | 3658 |
| 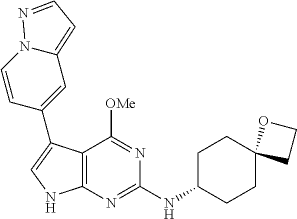 [SMILES: COc1nc(N[C@H]2CC[C@]3(CCO3)CC2)nc2[nH]cc(-c3ccn4nccc4c3)c12] | 3659 |
| 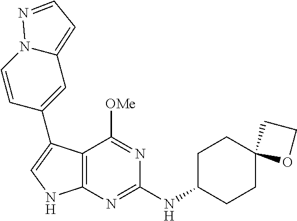 [SMILES: COc1nc(N[C@H]2CC[C@@]3(CCO3)CC2)nc2[nH]cc(-c3ccn4nccc4c3)c12] | 3660 |
| 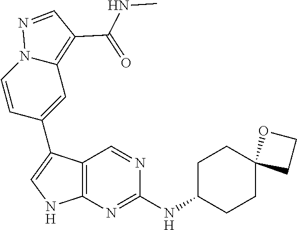 [SMILES: CNC(=O)c1cnn2ccc(-c3c[nH]c4nc(N[C@H]5CC[C@]6(CCO6)CC5)ncc34)cc12] | 3661 |
| 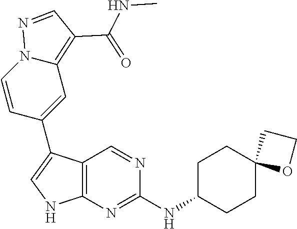 [SMILES: CNC(=O)c1cnn2ccc(-c3c[nH]c4nc(N[C@H]5CC[C@@]6(CCO6)CC5)ncc34)cc12] | 3662 |
| 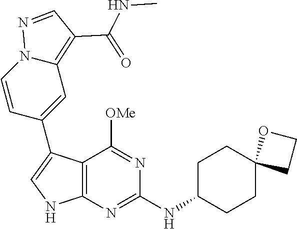 [SMILES: CNC(=O)c1cnn2ccc(-c3c[nH]c4nc(N[C@H]5CC[C@]6(CCO6)CC5)nc(OC)c34)cc12] | 3663 |
| 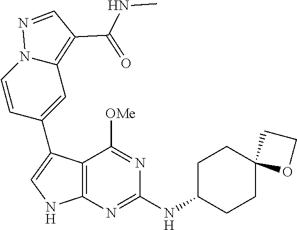 [SMILES: CNC(=O)c1cnn2ccc(-c3c[nH]c4nc(N[C@H]5CC[C@@]6(CCO6)CC5)nc(OC)c34)cc12] | 3664 |
| 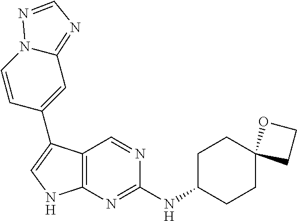 [SMILES: c1nc2cc(-c3c[nH]c4nc(N[C@H]5CC[C@]6(CCO6)CC5)ncc34)ccn2n1] | 3665 |
| 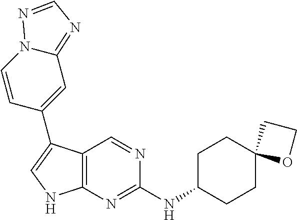 [SMILES: c1nc2cc(-c3c[nH]c4nc(N[C@H]5CC[C@@]6(CCO6)CC5)ncc34)ccn2n1] | 3666 |
| 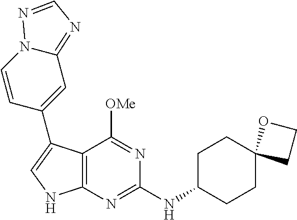 [SMILES: COc1nc(N[C@H]2CC[C@]3(CCO3)CC2)nc2[nH]cc(-c3ccn4ncnc4c3)c12] | 3667 |
| 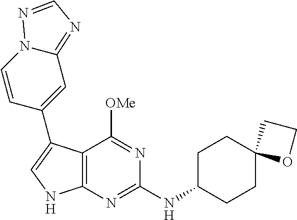 [SMILES: COc1nc(N[C@H]2CC[C@@]3(CCO3)CC2)nc2[nH]cc(-c3ccn4ncnc4c3)c12] | 3668 |
| 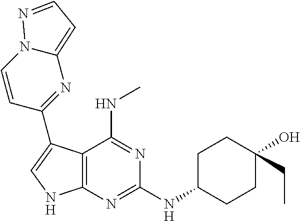 [SMILES: CC[C@]1(O)CC[C@@H](Nc2nc(NC)c3c(-c4ccn5nccc5n4)c[nH]c3n2)CC1] | 3669 |
| 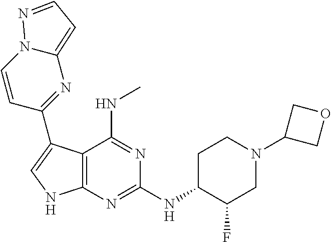 [SMILES: CNc1nc(N[C@@H]2CCN(C3COC3)C[C@@H]2F)nc2[nH]cc(-c3ccn4nccc4n3)c12] | 3670 |
| 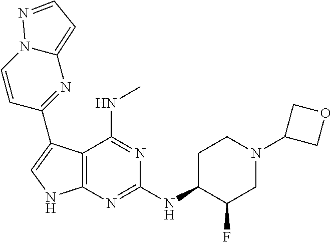 [SMILES: CNc1nc(N[C@H]2CCN(C3COC3)C[C@H]2F)nc2[nH]cc(-c3ccn4nccc4n3)c12] | 3671 |
| 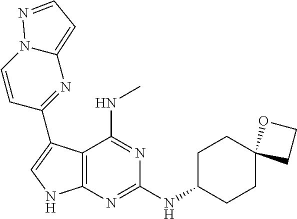 [SMILES: CNc1nc(N[C@H]2CC[C@]3(CCO3)CC2)nc2[nH]cc(-c3ccn4nccc4n3)c12] | 3672 |
| 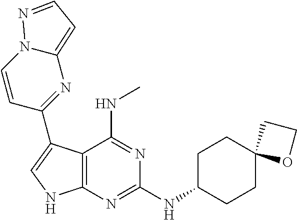 [SMILES: CNc1nc(N[C@H]2CC[C@@]3(CCO3)CC2)nc2[nH]cc(-c3ccn4nccc4n3)c12] | 3673 |
| 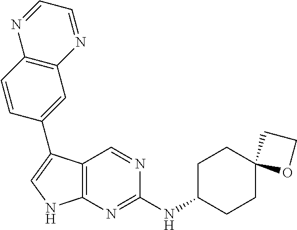 [SMILES: c1cnc2cc(-c3c[nH]c4nc(N[C@H]5CC[C@@]6(CCO6)CC5)ncc34)ccc2n1] | 3674 |
| 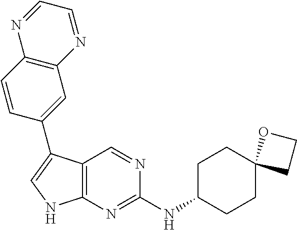 [SMILES: c1cnc2cc(-c3c[nH]c4nc(N[C@H]5CC[C@]6(CCO6)CC5)ncc34)ccc2n1] | 3675 |
| 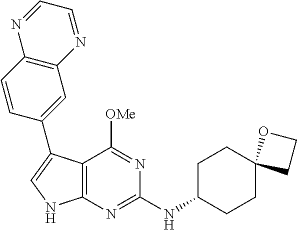 [SMILES: COc1nc(N[C@H]2CC[C@]3(CCO3)CC2)nc2[nH]cc(-c3ccc4nccnc4c3)c12] | 3676 |
| 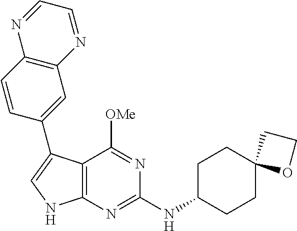 [SMILES: COc1nc(N[C@H]2CC[C@@]3(CCO3)CC2)nc2[nH]cc(-c3ccc4nccnc4c3)c12] | 3677 |

or a pharmaceutically acceptable salt thereof.

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

The compounds provided herein may also be useful in combination (administered together or sequentially) with other known agents.

Non-limiting examples of diseases which can be treated with a combination of a compound of Formulas I, Ia, Ib, and Ic, and other another active agent are colorectal cancer, ovarian cancer, hepatocellular carcinoma, head and neck squamous cell carcinoma, acute lymphoblastic leukemia (ALL), pancreatic cancer , brain tumors , acute megakaryoblastic leukemia (AMKL), diabetes mellitus, and osteoarthritis. For example, a compound of Formulas I, Ia, Ib, and Ic, can be combined with one or more chemotherapeutic compounds.

In some embodiments, hepatocellular carcinoma can be treated with a combination of a compound of Formulas I, Ia, Ib, and Ic, and one or more of the following drugs/therapies: sorafenib (Nexavar®); regorafenib (Stivarga®, Regonix®), nivolumab (Opdivo®); lenvatinib (Lenvima®); Pembrolizumab (Keytruda®); cabozantinib (Cometriq®, Cabometyx®); 5-fluorouracil (5-FU®); ramucirumab (Cyramza); combination of gemcitabine and oxaliplatin (GEMOX). Other therapies that can be performed in combination with a compound of Formulas I, Ia, Ib, and Ic, are i) transcatheter arterial chemoembolization (TACE) in combination with doxorubicin (DOXIC), cisplatin, or mitomycin C (Mitosol®, Mutamycin®, Jelmyto®; ii) low-dose brachytherapy.

In some embodiments, head and neck squamous cell carcinoma can be treated with a combination of a compound of Formulas I, Ia, Ib, and Ic, and one or more of the following drugs/therapies: TransOral Robotic Surgery (TORS); TORS with radiation therapy; larotrectinib) (Vitrakvi®; EGFR inhibitors, e.g., erlotinib (Tarceve), osimertinib (Tagrisse), neratinib) (Nerlynx®, gefitinib)(Iressa®, cetuximab (Erbitux®), panitumumab (Vectibix®), dacomitinib) (Vizimpro®, lapatinib)(Tykerb®, necitumumab (Portrazza), and vandetanib (Caprelsa).

In some embodiments, acute lymphoblastic leukemia (ALL) can be treated with a combination of a compound of Formulas I, Ia, Ib, and Ic, and one or more of the following drugs/therapies: remission induction therapy; consolidation therapy; nelarabine)(Arranon®; Asparaginase Erwinia Chrysanthemi)(Erwinaze®; Asparaginase Erwinia Chrysanthemi (Recombinant)-rywn (Rylaze®; calaspargase Pegol-mknl (Asparlas®; inotuzumab ozogamicin) (Besponsa®; blinatumomab)(Blincyto®; daunorubicin hydrochloride (Cerubidine®; clofarabine) (Clolar®; cyclophosphamide; methotrexate sodium)(Trexall ®; cytarabine)(Cytosar-U®; dasatinib (Sprycel®; dexamethasone; imatinib mesylate (Gleevec®; ponatinib hydrochloride (Iclusig®; mercaptopurine (Punnethol®), Purixan®; tisagenlecleucel (Kymriah®; vincristine sulfate liposome) (Marqibo®; pegaspargase (Oncaspar®; prednisone; daunorubicin hydrochloride (Rubidomycin); and vincristine sulfate.

In some embodiments, pancreatic cancer can be treated with a combination of a compound of Formulas I, Ia, Ib, and Ic, and one or more of the following drugs/therapies: ablation and embolization treatment; gemcitabine (Gemzar®; 5-fluorouracil)(5FU®); oxaliplatin (Eloxatin®; albumin-bound paclitaxel (Abraxane®; capecitabine (Xeloda®); cisplatin; irinotecan (Camptosar); liposomal Irinotecan (Onivyde); paclitaxel (Taxol)®, and docetaxel (Taxotere®).

In some embodiments, brain tumors can be treated with a combination of a compound of Formulas I, Ia, Ib, and Ic, and one or more of the following drugs/therapies: carmustine can be administered by way of a gliadel wafer; for glioblastoma and high-grade glioma, radiation therapy with daily low-dose temozolomide (Temodar®) followed by monthly doses of temozolomide after radiation therapy for 6 months to 1 year; lomustine (Gleostine®), procarbazine (Matulane®), and vincristine (Vincasar®), have been used along with radiation therapy; anti-angiogenesis therapy with bevacizumab (Avastin®, Mvasi®); and targeted therapy using larotrectinib (Vitrakvi®).

In some embodiments, acute megakaryoblastic leukemia (AMKL) can be treated with a combination of a compound of Formulas I, Ia, Ib, and Ic, and one or more of the following drugs/therapies: cytarabine (Cytosar-U®), etoposide (Vepesid), and anthracycline drugs. Anthracyclines include daunorubicin (Cerubidine®), idarubicin (Idamycin®, and mitoxantrone (Novantrone®).

In some embodiments, acute myeloid leukemia (AML) can be treated with a combination of a compound of Formulas I, Ia, Ib, and Ic, and one or more of the following drugs/thers: venetoclax and hypomethylating agents (e.g., decitabine, azacitidine), induction chemotherapy (cytarabine and an anthracycline (e.g., daunorubicin or idarubicin), all-trans-retinoic acid (ATRA) and either arsenic trioxide (ATO) monotherapy or an anthracycline), consolidation therapy (cytarabine).

In some embodiments, myelodysplastic syndrome (MDS) can be treated with a combination of a compound of Formulas I, Ia, Ib, and Ic, and one or more of the following drugs/therapies: 5-azacytidine, decitabine, lenalidomide, and decitabine/cedazuridine (Ingovi).

In some embodiments, colorectal cancer can be treated with a combination of a compound of Formulas I, Ia, Ib, and Ic, and one or more of the following drugs: 5-Fluorouracil (5-FU), which can be administered with the vitamin-like drug leucovorin (also called folinic acid); capecitabine (XELODA), irinotecan (CAMPOSTAR), oxaliplatin (ELOXATIN®). Examples of combinations of these drugs which could be further combined with a compound of Formula I are FOLFOX (5-FU, leucovorin, and oxaliplatin), FOLFIRI (5-FU, leucovorin, and irinotecan), FOLFOXIRI (leucovorin, 5-FU, oxaliplatin, and irinotecan) and CapeOx (Capecitabine and oxaliplatin). For rectal cancer, chemo with 5-FU or capecitabine combined with radiation may be given before surgery (neoadjuvant treatment).

In some embodiments, ovarian cancer can be treated with a combination of a compound of Formulas I, Ia, Ib, and Ic, and one or more of the following drugs: Topotecan, Liposomal doxorubicin (DOXIC), Gemcitabine (GEMZAle), Cyclophosphamide) (CYTOXAN®, Vinorelbine (NAVELBINE®), Ifosfamide (IFEX®), Etoposide (VP-16), Altretamine (HEXALEN®), Capecitabine (XELODA®), Irinotecan (CPT-11, CAMPTOSAR®), Melphalan, Pemetrexed (ALIMTA®) and Albumin bound paclitaxel (nab-paclitaxel,) ABRAXANE®. Examples of combinations of these drugs which could be further combined with a compound of Formulas I, Ia, Ib, and Ic, are TIP (paclitaxel [Taxol], ifosfamide, and cisplatin), VeIP (vinblastine, ifosfamide, and cisplatin) and VIP (etoposide [VP-16], ifosfamide, and cisplatin). Ovarian cancer can also be treated with a combination of a compound of Formula (I) and immune checkpoint blockade (ICB) therapy.

In some embodiments, a compound of Formulas I, Ia, Ib, and Ic, can be used to treat cancer in combination with any of the following methods: (a) Hormone therapy such as aromatase inhibitors, LHRH [luteinizing hormone-releasing hormone] analogs and inhibitors, and others; (b) Ablation or embolization procedures such as radiofrequency ablation (RFA), ethanol (alcohol) ablation, microwave thermotherapy and cryosurgery (cryotherapy); (c) Chemotherapy using alkylating agents such as cisplatin and carboplatin, oxaliplatin, mechlorethamine, cyclophosphamide, chlorambucil and ifosfamide; (d) Chemotherapy using anti-metabolites such as azathioprine and mercaptopurine; (e) Chemotherapy using plant alkaloids and terpenoids such as vinca alkaloids (i.e. Vincristine, Vinblastine, Vinorelbine and Vindesine) and taxanes; (f) Chemotherapy using podophyllotoxin, etoposide, teniposide and docetaxel; (g) Chemotherapy using topoisomerase inhibitors such as irinotecan, topotecan, amsacrine, etoposide, etoposide phosphate, and teniposide; (h) Chemotherapy using cytotoxic antibiotics such as actinomycin, anthracyclines, doxorubicin, daunorubicin, valrubicin, idarubicin, epirubicin, bleomycin, plicamycin and mitomycin; (i) Chemotherapy using tyrosine-kinase inhibitors such as Imatinib mesylate (GLEEVEC®), also known as STI-571), Gefitinib (Iressa, also known as ZD1839), Erlotinib (marketed as)TARCEVA®, Bortezomib (VELCADE®), tamoxifen , tofacitinib, crizotinib, Bcl-2 inhibitors (e.g. obatoclax, navitoclax (ABT-263), oblimersen (G3139), venetoclax (ABT-199), Gossypol), PARP inhibitors (e.g. Iniparib, Olaparib, Rucaparib, Niraparib, Talazoparib), PI3K inhibitors (e.g. perifosine in a phase III trial), VEGF Receptor 2 inhibitors (e.g. Apatinib), AN-152, (AEZS-108), Braf inhibitors (e.g. vemurafenib, dabrafenib and LGX818), MEK inhibitors (e.g. trametinib and MEK162), CDK inhibitors, (e.g. PD-0332991), salinomycin and Sorafenib; (j) Chemotherapy using monoclonal antibodies such as Rituximab (marketed as MABTHERA® or RITUXAN®), Trastuzumab (Herceptin also known as ErbB2), Cetuximab (marketed as)ERBITUX®, and Bevacizumab (marketed as AVASTIN®); (k) Chemotherapy using KRAS G12C inhibitors such as sotorasib (Lumakras® and Lumykras®), adagrasib (MRTX849), and ARS-3248 (Wellspring Biosciences); (1) Chemotherapy using checkpoint inhibitor therapy such as Ipilimumab (Yervoy®), Nivolumab (Opdive), Pembrolizumab (Keytruda), Atezolizumab (Tecentrie), Avelumab (Bavencio), Durvalumab (Imfinzi), Cemiplimab)(Libtayo®, and Spartalizumab (PDR001); (m) Chemotherapy using antibody-drug conjugates (ADC) such as Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, and Loncastuximab tesirine; (n) Chemotherapy using proteasome inhibitors such as carfilzomib, lactacystin, disulfiram, salinosporamide A (marizomib), oprozomib, delanzomib, epoxomicin, MG132, 13-hydroxy 13-methylbutyric acid (HMB), bortezomib, ixazomib (alone or in in combination with lenalidomide and dexamethasone); and (o) radiation therapy.

In some embodiments, a compound of Formulas I, Ia, Ib, and Ic, can be used to treat diabetes mellitus in combination with any of the following methods: (a) injections of insulin; (b) biguanides such as metformin (Glucophage), phenformin (DBI), and buformin; (c) thiazolidinediones (TZDs) such as rosiglitazone (Avandia), pioglitazone (Actos), and yroglitazone (Rezulin); (d) lyn kinase activators such as glimepiride (Amaryl®) and tolimidone (MLR-1023); (e) secretagogues such as sulfonylureas (non-limiting examples are acetohexamide, carbutamide, chlorpropamide, glycyclamide (tolcyclamide), metahexamide, tolazamide, tolbutamide, glibenclamide (glyburide), glibornuride, gliclazide, glipizide, gliquidone, glisoxepide, glyclopyramide, and glimepiride) and meglitinides (nonlimiting examples are repaglinide (Prandin), nateglinide (Starlix), and mitiglinide (Glufast)); (f) alpha-glucosidase inhibitors such as acarbose (Glucobay, Precose, Prandase), miglitol (Glyset), and voglibose; (g) injectable incretin mimetics such as glucagon-like peptide-1 (GLP-1) and gastric inhibitory peptide (glucose-dependent insulinotropic peptide, GIP), nonlimiting examples of injectable glucagon-like peptide (GLP) analogs and agonists are exenatide (Exendin-4, marketed as Byetta), liraglutide (Victoza, Saxenda), taspoglutide, lixisenatide (Lyxumia), Semaglutide (Ozempic, Rybelsus), dulaglutide (Trulicity), albiglutide (Tanzeum), nonlimiting examples of dipeptidyl peptidase-4 (DPP-4) inhibitors are sitagliptin (Januvia), vildagliptin (Galvus), saxagliptin (Onglyza), linagliptin (Tradjenta), gemigliptin (Zemiglo), anagliptin (Suiny), teneligliptin (Tenelia), alogliptin (Nesina, Vipidia, Kazano, Vipidomet (with metformin), Oseni, Incresync (with pioglitazone)), trelagliptin (Zafatek, Wedica), omarigliptin (MK-3102), evogliptin (Suganon, Evodine), gosogliptin (Saterex), and dutogliptin; (h) injectable amylin analogues such as pramlintide (Symlin); (i) glycosurics (SGLT2 inhibitors) such as canagliflozin (Invokana, Sulisent, Prominad), dapagliflozin (Forxiga, Farxiga, Edistride), empagliflozin (Jardiance, Sciampa-M), ertugliflozin (Steglatro), ipragliflozin (Suglat), luseogliflozin (Lusefi), remogliflozin etabonate (pro-drug of remogliflozin), sergliflozin etabonate (GW869682X), sotagliflozin (Zynquista), and tofogliflozin (CSG452).

In some embodiments, a compound of Formulas I, Ia, Ib, and Ic, can be used to treat osteoarthritis in combination with any of the following methods: (a) injections of a Wnt signaling pathway inhibitor (e.g. lorecivivint); (b) Nonsteroidal anti-inflammatory drugs (NSAIDs) such as ibuprofen, naproxen, aspirin and acetaminophen; (c) physical therapy; (d) injections of corticosteroid medications; (e) injections of hyaluronic acid derivatives (e.g. Hyalgan, Synvisc); (f) narcotics, like codeine; (g) in combination with braces and/or shoe inserts or any device that can immobilize or support your joint to help you keep pressure off it (e.g., splints, braces, shoe inserts or other medical devices); (h) realigning bones (osteotomy); (i) joint replacement (arthroplasty); and (i) in combination with a chronic pain class.

In some embodiments, a compound of Formulas I, Ia, Ib, and Ic, can be used to treat Alzheimer's disease in combination with aducanumab (Aduhelm™); acetylcholinesterase inhibitors, e.g., tacrine, rivastigmine (Exelon), galantamine (Razadyne® and GalantaMind™), and donepezil (Aricept®); and memantine (Axura®, Ebixa®, Namenda®).

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release, or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants, and the like, for prolonged and/or timed, pulsed administration at a predetermined rate.

The compounds can be administered either alone or in combination with a conventional pharmaceutical carrier, excipient, or the like. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol, or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the unit dosage of compounds of Formulas I, Ia, Ib, and Ic, is about 0.25 mg/Kg to about 50 mg/Kg in humans.

In some embodiments, the unit dosage of compounds of Formulas I, Ia, Ib, and Ic, is about 0.25 mg/Kg to about 20 mg/Kg in humans.

In some embodiments, the unit dosage of compounds of Formulas I, Ia, Ib, and Ic, is about 0.50 mg/Kg to about 19 mg/Kg in humans.

In some embodiments, the unit dosage of compounds of Formulas I, Ia, Ib, and Ic, is about 0.75 mg/Kg to about 18 mg/Kg in humans.

In some embodiments, the unit dosage of compounds of Formulas I, Ia, Ib, and Ic, is about 1.0 mg/Kg to about 17 mg/Kg in humans.

In some embodiments, the unit dosage of compounds of Formulas I, Ia, Ib, and Ic, is about 1.25 mg/Kg to about 16 mg/Kg in humans.

In some embodiments, the unit dosage of compounds of Formulas I, Ia, Ib, and Ic, is about 1.50 mg/Kg to about 15 mg/Kg in humans.

In some embodiments, the unit dosage of compounds of Formulas I, Ia, Ib, and Ic, is about 1.75 mg/Kg to about 14 mg/Kg in humans.

In some embodiments, the unit dosage of compounds of Formulas I, Ia, Ib, and Ic, is about 2.0 mg/Kg to about 13 mg/Kg in humans.

In some embodiments, the unit dosage of compounds of Formulas I, Ia, Ib, and Ic, is about 3.0 mg/Kg to about 12 mg/Kg in humans.

In some embodiments, the unit dosage of compounds of Formulas I, Ia, Ib, and Ic, is about 4.0 mg/Kg to about 11 mg/Kg in humans.

In some embodiments, the unit dosage of compounds of Formulas I, Ia, Ib, and Ic, is about 5.0 mg/Kg to about 10 mg/Kg in humans.

In some embodiments, the compositions are provided in unit dosage forms suitable for single administration.

In some embodiments, the compositions are provided in unit dosage forms suitable for twice a day administration.

In some embodiments, the compositions are provided in unit dosage forms suitable for three times a day administration.

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable and could be higher if the composition is a solid or suspension, which could be subsequently diluted to the above percentages.

In some embodiments, the composition comprises about 0.1-10% of the active agent in solution.

In some embodiments, the composition comprises about 0.1-5% of the active agent in solution.

In some embodiments, the composition comprises about 0.1-4% of the active agent in solution.

In some embodiments, the composition comprises about 0.15-3% of the active agent in solution.

In some embodiments, the composition comprises about 0.2-2% of the active agent in solution.

In some embodiments, the compositions are provided in dosage forms suitable for continuous dosage by intravenous infusion over a period of about 1-96 hours.

In some embodiments, the compositions are provided in dosage forms suitable for continuous dosage by intravenous infusion over a period of about 1-72 hours.

In some embodiments, the compositions are provided in dosage forms suitable for continuous dosage by intravenous infusion over a period of about 1-48 hours.

In some embodiments, the compositions are provided in dosage forms suitable for continuous dosage by intravenous infusion over a period of about 1-24 hours.

In some embodiments, the compositions are provided in dosage forms suitable for continuous dosage by intravenous infusion over a period of about 1-12 hours.

In some embodiments, the compositions are provided in dosage forms suitable for continuous dosage by intravenous infusion over a period of about 1-6 hours.

In some embodiments, these compositions can be administered by intravenous infusion to humans at doses of about 5 mg/m² to about 300 mg/m².

In some embodiments, these compositions can be administered by intravenous infusion to humans at doses of about 5 mg/m² to about 200 mg/m².

In some embodiments, these compositions can be administered by intravenous infusion to humans at doses of about 5 mg/m² to about 100 mg/m².

In some embodiments, these compositions can be administered by intravenous infusion to humans at doses of about 10 mg/m² to about 50 mg/m².

In some embodiments, these compositions can be administered by intravenous infusion to humans at doses of about 50 mg/m² to about 200 mg/m².

In some embodiments, these compositions can be administered by intravenous infusion to humans at doses of about 75 mg/m² to about 175 mg/m².

In some embodiments, these compositions can be administered by intravenous infusion to humans at doses of about 100 mg/m² to about 150 mg/m².

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 p.m are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formulas I, Ia, Ib, and Ic disclosed herein intended for respiratory delivery (either systemic or local) can be administered as aqueous formulations, as non-aqueous solutions, or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formulas I, Ia, Ib, and Ic disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the disclosure can be incorporated into a gel formulation (e.g., U.S. Pat. Nos. 4,474,752 and 6,911,211).

In some embodiments, compounds of Formulas I, Ia, Ib, and Ic disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid compound of Formulas I, Ia, Ib, and Ic can be delivered from the reservoir of an external or internal implanted pumping system.

Formulations of the disclosure also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formulas I, Ia, Ib, and Ic are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration of the drug (either as a solution, colloid, suspension or a complex) can be prepared by mixing a compound provided herein with a suitable non-irritating excipient that is solid at ordinary temperatures but liquid at the rectal temperature and will therefore melt or erode/dissolve in the rectum and release the compound. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter, is first melted.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of glioblastoma, ovarian, breast, pancreatic cancers, acute lymphoblastic leukemia, acute megakaryoblastic leukemia, chronic myeloid leukemia, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, Autism, Dementia, Epilepsy, Huntington's Disease, and Multiple Sclerosis.

### Methods of Treatment

The compounds and compositions provided herein can be used as inhibitors of DYRK1A, and thus can be used to treat a variety of disorders and diseases in which over expression of DYRK1A is implicated, such as cancer and neurological conditions/disorders/diseases. Non-limiting examples of diseases which can be treated with the compounds and compositions provided herein include a variety of cancers, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, tauopathies (e.g., Pick's disease, progressive supranuclear palsy, corticobasal degeneration, argyrophilic grain disease, globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

The gene encoding DYRK1A is located on chromosome 21, within the Down syndrome critical region (DSCR), the triploidy of which is responsible for most Down syndrome-associated deficiencies (FEBS Journal (2011), 278, 246-256). There is considerable genetical and pharmacological evidence showing that the mere 1.5-fold overexpression of DYRK1A is responsible for most cognitive deficits observed in Down syndrome patients (Pharmacology & Therapeutics (2019), 194, 199-221 and Brain Science (2018), 8(10), 187). Genetical normalization of DYRK1A levels or pharmacological inhibition of its catalytic activity restores cognitive functions. The development of pharmacological inhibitors of DYRK1A is a major avenue for the treatment of cognitive deficits associated with Down syndrome.

DYRK1A and DYRK1B are utilized during human cytomegalovirus (HCMV) placental replication. Inhibition of DYRKs prevent replication of various viruses, including hepatitis C virus (HCV), human cytomegalovirus (HCMV), human immunodeficiency virus type 1 (HIV-1), and herpes simplex virus 1 (HSV-1) (Journal of Virology (2020), 94(6) and PLoS ONE (2015), 10, e0144229).

There is a growing body of evidence showing that DYRK1A/1B inhibitors induce the proliferation of insulin-producing pancreatic β-cells, making DYRK1A/1B kinases attractive therapeutic targets for (3-cell regeneration for both type 1 and type 2 diabetes mellitus and gestational diabetes (Nature Communications (2015), 6(8372); Diabetes (2016), 65(6), 1660-1671; JCI Insight (2020), 5(1), e132594; Science Translational Medicine (2020), 12(530); International Journal of Molecular Sciences (2021), 22(16), 9083; and Journal of Medicinal Chemistry (2021), 64(6), 2901-2922). Other forms of diabetes that may be treated with DYRK inhibitors are maturity onset diabetes of the young (MODY, monogenic diabetes), cases of diabetes that are caused by the body's tissue receptors not responding to insulin, double diabetes (when a type 1 diabetic becomes insulin resistant), diabetes associated with excessive secretion of insulin-antagonistic hormones, malnutrition-related diabetes mellitus (ICD-10 code E12), and diabetes caused by any genetic mutations (autosomal or mitochondrial) that leads to defects in beta cell function.

There is abundant literature linking DYRK1A with solid cancers and leukemias (Pharmacology & Therapeutics (2015), 151, 87-98; Cancers (2020), 12(8), 2106; and Cellular and Molecular Life Sciences (2021), 78, 603-619). The most prominent examples are pancreatic cancer (Gut (2019), 68(8), 1465-1476 and Gene (2020), 758, 144960), brain tumors, glioblastoma (Journal of Clinical Investigation (2013), 123(6), 2475-2487), acute megakaryoblastic leukemia (AMKL) (Journal of Clinical Investigation (2012), 122(3), 948-962), and acute lymphoblastic leukemia (ALL) (Journal of Clinical Investigation (2021), 131(1), e135937). Other cancers linked to DYRK1A are ovarian (Frontiers in Oncology (2021), 11, 637193), head and neck squamous cell carcinoma (Scientific Reports (2016), 6, 36132), hepatocellular carcinoma (Cell Death & Disease (2021), 12, 125), DYRK1A regulates DNA damage response (Scientific Reports (2019), 9, 6014 and Scientific Reports (2019), 9, 6539). In some situations, DYRK1A appears to function as a tumor-suppressor protein (Molecular & Cellular Oncology (2015), 2(1), e970048 and Nature (2016), 529, 172-177).

Other cancers can also be treated with the compounds and compositions described herein.

More particularly, cancers that may be treated by the compounds, compositions and methods described herein include, but are not limited to, the following:

1) Breast cancers, including, for example ER⁺breast cancer, ER⁻ breast cancer, her2⁻ breast cancer, her2⁺breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; chemoresistant breast cancers (TNBC), and miscellaneous malignant neoplasms. Further examples of breast cancers can include luminal A, luminal B, basal A, basal B, and triple negative breast cancer, which is estrogen receptor negative (ER), progesterone receptor negative, and her2 negative (her2). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; chemoresistant small cell lung cancer (SCLC), and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; colon cancers with APC gene mutations; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, oligodendrocytoma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial cancers (e.g., carcinoma, endometrioid adenocarcinoma, serous carcinoma, clear cell carcinoma, mucinous carcinomas, mixed or undifferentiated carcinoma (including mixed Mullerian tumor), endometrial stromal sarcoma, squamous cell carcinoma of the endometrium, urothelial carcinoma, endometrial cancer with CTNNB1 mutations); cancers of the cervix, e.g., cervical carcinoma, and pre tumor cervical dysplasia; cancers of the ovaries, e.g., BRCA-mutant ovarian cancer, surface epithelial-stromal tumors (epithelial ovarian cancer (Type 1 (endometroid, mucinous, clear cell, low grade serous) or Type 2 (poorly differentiated, carcinosarcoma, and high grade serous))), ovarian carcinoma, including serous cystadenocarcinoma, mucinous cystadenocarcinoma, endometrioid tumors, small cell ovarian cancer (small cell ovarian cancer of hypercalcemic type, small cell ovarian cancer of pulmonary type) unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma, primary fallopian tube cancer; Primary peritoneal cancer (also known as serous surface papillary carcinoma, primary peritoneal carcinoma, extra-ovarian serous carcinoma, primary serous papillary carcinoma, and psammomacarcinoma).

10) Hematologic cancers, including, for example, cancers of the blood, e.g., acute myeloid leukemia, chronic myeloid leukemia, myelodysplastic syndromes (refractory cytopenia with unilineage dysplasia (refractory anemia, refractory neutropenia, and refractory thrombocytopenia), refractory anemia with ring sideroblasts, refractory cytopenia with multilineage dysplasia, refractory anemias with excess blasts I and II, refractory cytopenia of childhood), and myeloproliferative neoplasms, acute lymphoblastic leukemia, chronic lymphocytic leukemia, myeloproliferative diseases, multiple myeloma, myelodysplastic syndrome, myelodysplastic—myeloproliferative diseases, Hodgkin's lymphoma, non-Hodgkin's lymphoma (malignant lymphoma) and WaldenstrOm's macroglobulinemia.

11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.

12) Adrenal gland cancers, including, for example, neuroblastoma.

13) Soft-tissue sarcomas (STS) such as fibrosarcoma, malignant fibrous histiocytoma, dermatofibrosarcoma, liposarcoma, rhabdomyosarcoma, leiomyosarcoma, hemangiosarcoma, Kaposi's sarcoma, lymphangiosarcoma, synovial sarcoma, malignant peripheral nerve sheath tumors (also called neurofibrosarcomas, malignant schwannomas, and neurogenic sarcomas), neurofibrosarcoma, extraskeletal chondrosarcoma, extraskeletal osteosarcoma, extraskeletal myxoid chondrosarcoma, extraskeletal mesenchymal, embryonal, alveolar soft part sarcoma, and infantile hemangio-pericytoma.

More particularly, tumors of the central nervous system that may be treated by the compounds, compositions and methods described herein include:

1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.

2) Oligodendroglial tumors, e.g., oligodendroglioma and anaplastic oligodendroglioma.

3) Oligoastrocytic tumors, e.g., oligoastrocytoma and anaplastic oligoastrocytoma.

4) Ependymal tumors, e.g., subependymoma, myxopapillary ependymoma, ependymoma, (cellular, papillary, clear cell, tanycytic), and anaplastic (malignant) ependymoma.

5) Choroid plexus tumors, e.g., choroid plexus papilloma, atypical choroid plexus papilloma, and choroid plexus carcinoma.

6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.

7) Pineal tumors, e.g., pineocytoma, pineoblastoma, papillary tumors of the pineal region, and pineal parenchymal tumor of intermediate differentiation.

8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.

9) Neuroblastic tumors, e.g., olfactory (esthesioneuroblastoma), olfactory neuroepithelioma, and neuroblastomas of the adrenal gland and sympathetic nervous system.

10) Glial tumors, e.g., astroblastoma, chordoid glioma of the third ventricle, and angiocentric glioma.

11) Tumors of cranial and paraspinal nerves, e.g., schwannoma, neurofibroma Perineurioma, and malignant peripheral nerve sheath tumor.

12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.

13) Tumors of the hematopoietic system, e.g., malignant Lymphomas, plasmocytoma, and granulocytic sarcoma.

14) Germ cell tumors, e.g., germinoma, embryonal carcinoma, yolk sac tumor, choriocarcinoma, teratoma, and mixed germ cell tumors.

15) Tumors of the sellar region, e.g., craniopharyngioma, granular cell tumor, pituicytoma, and spindle cell oncocytoma of the adenohypophysis.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

There is mounting evidence for a role of DYRK1A in the onset of Alzheimer's Disease (Future Medicinal Chemistry (2016), 8(6), 681-696 and European Journal of Medicinal Chemistry (2018), 158, 559-592). DYRK1A phosphorylates key substrates involved in Alzheimer's Disease and dementia: Tau, septin 4, amyloid precursor protein (APP), presenilin 1, neprilysin, Munc18-1, α-synuclein, RCAN1, and β-tubulin. By modulating alternative splicing of Tau exon 10, DYRK1A favors the production of the 3R-Tau splice isoform (characteristic for DS/AD/tauopathy) over the 4R-Tau isoform (Journal ofBiological Chemistry (2015), 290, 15219-15237).

Genome-wide association studies (GWAS) have revealed that DYRK1A is a risk factor for Parkinson's Disease (The Lancet Neurology (2019), 18(12), 1091-1102). DYRK1A phosphorylates key factors for Parkinson's Disease such as parkin, septin 4, and a-synuclein.

Upregulation of micro-RNAs specific for Parkinson's Disease targets DYRK1A expression. There is further evidence that DYRK1A expression is increased in Parkinson's Disease and in Pick's disease (Neurobiology of Disease (2005), 20(2), 392-400).

The compounds and compositions provided herein can be used as inhibitors and/or modulators of the enzyme DYRK1A, and thus can be used to treat a variety of disorders and diseases associated with tau protein, including, but not limited to, Alzheimer's disease, amyotrophic lateral sclerosis (ALS), down syndrome, frontotemporal dementia (FTD) including FTD with Parkinsonism-17 (FTDP-17), behavioural variant frontotemporal dementia (bvFTD), FTD in patients with motor neuron disease (MND) (typically amyotrophic lateral sclerosis, also called FTD-ALS), corticobasal degeneration (CBD) (also called corticobasal ganglionic degeneration), progressive supranuclear palsy, primary progressive aphasia (PPA), globular glial tauopathy (GGT), myotonic dystrophy type 1 (DM1) (also called Steinert disease), myotonic dystrophy type 2 (DM2) (also called proximal myotonic myopathy), Guam complex, argyrophilic grain disease, dementia pugilistica, post-encephalitic parkinsonism, Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

Non-limiting examples of neurological disorders (e.g., neurological conditions and neurological diseases) which can be treated with the compounds and compositions provided herein include Alzheimer's disease, aphasia, apraxia, arachnoiditis, ataxia telangiectasia, attention deficit hyperactivity disorder, auditory processing disorder, autism, alcoholism, Bell's palsy, bipolar disorder, brachial plexus injury, Canavan disease, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, cerebral vasculitis, cervical spinal stenosis, Charcot-Marie-Tooth disease, Chiari malformation, chronic fatigue syndrome, chronic inflammatory demyelinating polyneuropathy (CIDP), chronic pain, Coffin—Lowry syndrome, complex regional pain syndrome, compression neuropathy, congenital facial diplegia, corticobasal degeneration, cranial arteritis, craniosynostosis, Creutzfeldt-Jakob disease, cumulative trauma disorder, Cushing's syndrome, cytomegalic inclusion body disease (CIBD), Dandy-Walker syndrome, Dawson disease, De Morsier's syndrome, Dejerine-Klumpke palsy, Dejerine-Sottas disease, delayed sleep phase syndrome, dementia, dermatomyositis, developmental dyspraxia, diabetic neuropathy, diffuse sclerosis, Dravet syndrome, dysautonomia, dyscalculia, dysgraphia, dyslexia, dystonia, empty sella syndrome, encephalitis, encephalocele, encephalotrigeminal angiomatosis, encopresis, epilepsy, Erb's palsy, erythromelalgia, essential tremor, Fabry's disease, Fahr's syndrome, familial spastic paralysis, febrile seizure, Fisher syndrome, Friedreich's ataxia, fibromyalgia, Foville's syndrome, Gaucher's disease, Gerstmann's syndrome, giant cell arteritis, giant cell inclusion disease, globoid cell leukodystrophy, gray matter heterotopia, Guillain-Barré syndrome, HTLV-1 associated myelopathy, Hallervorden-Spatz disease, hemifacial spasm, hereditary spastic paraplegia, heredopathia atactica polyneuritiformis, herpes zoster oticus, herpes zoster, Hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile Refsum disease, infantile spasms, inflammatory myopathy, intracranial cyst, intracranial hypertension, Joubert syndrome, Karak syndrome, Kearns-Sayre syndrome, Kennedy disease, Kinsbourne syndrome, Klippel Feil syndrome, Krabbe disease, Kugelberg-Welander disease, kuru, Lafora disease, Lambert-Eaton myasthenic syndrome, Landau-Kleffner syndrome, lateral medullary (Wallenberg) syndrome, Leigh's disease, Lennox-Gastaut syndrome, Lesch-Nyhan syndrome, leukodystrophy, Lewy body dementia, lissencephaly, locked-in syndrome, Lou Gehrig's disease, lumbar disc disease, lumbar spinal stenosis, Lyme disease, Machado-Joseph disease (Spinocerebellar ataxia type 3), macrencephaly, macropsia, megalencephaly, Melkersson-Rosenthal syndrome, Meniere's disease, meningitis, Menkes disease, metachromatic leukodystrophy, microcephaly, micropsia, Miller Fisher syndrome, misophonia, mitochondrial myopathy, Mobius syndrome, monomelic amyotrophy, motor neuron disease, motor skills disorder, Moyamoya disease, mucopolysaccharidoses, multi-infarct dementia, multifocal motor neuropathy, multiple sclerosis, multiple system atrophy, muscular dystrophy, myalgic encephalomyelitis, myasthenia gravis, myelinoclastic diffuse sclerosis, myoclonic Encephalopathy of infants, myoclonus, myopathy, myotubular myopathy, myotonia congenital, narcolepsy, neurofibromatosis, neuroleptic malignant syndrome, lupus erythematosus, neuromyotonia, neuronal ceroid lipofuscinosis, Niemann-Pick disease, O′Sullivan-McLeod syndrome, occipital Neuralgia, occult Spinal Dysraphism Sequence, Ohtahara syndrome, olivopontocerebellar atrophy, opsoclonus myoclonus syndrome, optic neuritis, orthostatic hypotension, palinopsia, paresthesia, Parkinson's disease, paramyotonia Congenita, paraneoplastic diseases, paroxysmal attacks, Parry-Romberg syndrome, Pelizaeus-Merzbacher disease, periodic paralyses, peripheral neuropathy, photic sneeze reflex, phytanic acid storage disease, Pick's disease, polymicrogyria (PMG), polymyositis, porencephaly, post-polio syndrome, postherpetic neuralgia (PHN), postural hypotension, Prader-Willi syndrome, primary lateral sclerosis, prion diseases, progressive hemifacial atrophy, progressive multifocal leukoencephalopathy, progressive supranuclear palsy, pseudotumor cerebri, Ramsay Hunt syndrome type I, Ramsay Hunt syndrome type II, Ramsay Hunt syndrome type III, Rasmussen's encephalitis, reflex neurovascular dystrophy, Refsum disease, restless legs syndrome, retrovirus-associated myelopathy, Rett syndrome, Reye's syndrome, rhythmic movement disorder, Romberg syndrome, Saint Vitus dance, Sandhoff disease, schizophrenia, Schilder's disease, schizencephaly, sensory integration dysfunction, septo-optic dysplasia, Shy-Drager syndrome, Sjogren's syndrome, snatiation, Sotos syndrome, spasticity, spina bifida, spinal cord tumors, spinal muscular atrophy, spinocerebellar ataxia, Steele-Richardson-Olszewski syndrome, Stiff-person syndrome, stroke, Sturge-Weber syndrome, subacute sclerosing panencephalitis, subcortical arteriosclerotic encephalopathy, superficial siderosis, Sydenham's chorea, syncope, synesthesia, syringomyelia, tarsal tunnel syndrome, tardive dyskinesia, tardive dysphrenia, Tarlov cyst, Tay-Sachs disease, temporal arteritis, tetanus, tethered spinal cord syndrome, Thomsen disease, thoracic outlet syndrome, tic douloureux, Todd's paralysis, Tourette syndrome, toxic encephalopathy, transient ischemic attack, transmissible spongiform encephalopathies, transverse myelitis, tremor, trigeminal neuralgia, tropical spastic paraparesis, trypanosomiasis, tuberous sclerosis, ubisiosis, Von Hippel-Lindau disease (VHL), Viliuisk Encephalomyelitis (VE), Wallenberg's syndrome, Werdnig, Hoffman disease, west syndrome, Williams syndrome, Wilson's disease, and Zellweger syndrome.

The compounds and compositions may also be useful in the inhibition of the development of invasive cancer, tumor angiogenesis and metastasis.

In some embodiments, the pharmaceutical composition comprises a therapeutically effective amount of a compound of Formulas I, Ia, Ib, and Ic, or a pharmaceutically acceptable salt thereof, and a pharmaceutically acceptable excipient.

In some embodiments, the disorder or disease is cancer.

In some embodiments, the disorder or disease is metastatic melanoma.

In some embodiments, the disorder or disease is tendon regeneration.

In some embodiments, the disorder or disease is diabetes.

In some embodiments, the disorder or disease is degenerative disc disease.

In some embodiments, the disorder or disease is osteoarthritis.

In some embodiments, the disorder or disease is a viral infection.

In some embodiments, the disorder or disease is a neurological disorder.

In some embodiments, the disorder or disease is Alzheimer's disease.

In some embodiments, the disorder or disease is osteoarthritis.

In some embodiments, the patient is a human.

In some embodiments, the cancer is chosen from: hepatocellular carcinoma, colon cancer, breast cancer, pancreatic cancer, chronic myeloid leukemia (CML), chronic myelomonocytic leukemia, chronic lymphocytic leukemia (CLL), acute myeloid leukemia, acute lymphocytic leukemia, Hodgkin lymphoma, lymphoma, sarcoma, and ovarian cancer.

In some embodiments, the cancer is chosen from: lung cancer—non-small cell, lung cancer—small cell, multiple myeloma, nasopharyngeal cancer, neuroblastoma, osteosarcoma, penile cancer, pituitary tumors, prostate cancer, retinoblastoma, synovial sarcoma, rhabdomyosarcoma, salivary gland cancer, skin cancer—basal and squamous cell, skin cancer—melanoma, small intestine cancer, stomach (gastric) cancers, testicular cancer, thymus cancer, thyroid cancer, uterine sarcoma, vaginal cancer, vulvar cancer, laryngeal or hypopharyngeal cancer, kidney cancer, Kaposi sarcoma, gestational trophoblastic disease, gastrointestinal stromal tumor, gastrointestinal carcinoid tumor, gallbladder cancer, eye cancer (melanoma and lymphoma), Ewing tumor, esophagus cancer, endometrial cancer, colorectal cancer, cervical cancer, brain or spinal cord tumor, bone metastasis, bone cancer, bladder cancer, bile duct cancer, anal cancer and adrenal cortical cancer.

In some embodiments, the cancer is hepatocellular carcinoma; in some embodiments, the cancer is colon cancer; in some embodiments, the cancer is colorectal cancer; in some embodiments, the cancer is breast cancer; in some embodiments, the cancer is pancreatic cancer; in some embodiments, the cancer is chronic myeloid leukemia (CML); in some embodiments, the cancer is chronic myelomonocytic leukemia; in some embodiments, the cancer is chronic lymphocytic leukemia (CLL); in some embodiments, the cancer is acute myeloid leukemia; in some embodiments, the cancer is acute lymphocytic leukemia; in some embodiments, the cancer is Hodgkin lymphoma; in some embodiments, the cancer is lymphoma; in some embodiments, the cancer is sarcoma; in some embodiments, the cancer is ovarian cancer; in some embodiments, the cancer is lung cancer—non-small cell; in some embodiments, the cancer is lung cancer—small cell; in some embodiments, the cancer is multiple myeloma; in some embodiments, the cancer is nasopharyngeal cancer; in some embodiments, the cancer is neuroblastoma; in some embodiments, the cancer is osteosarcoma; in some embodiments, the cancer is penile cancer; in some embodiments, the cancer is pituitary tumors; in some embodiments, the cancer is prostate cancer; in some embodiments, the cancer is retinoblastoma; in some embodiments, the cancer is rhabdomyosarcoma; in some embodiments, the cancer is salivary gland cancer; in some embodiments, the cancer is skin cancer—basal and squamous cell; in some embodiments, the cancer is skin cancer—melanoma; in some embodiments, the cancer is small intestine cancer; in some embodiments, the cancer is stomach (gastric) cancers; in some embodiments, the cancer is testicular cancer; in some embodiments, the cancer is thymus cancer; in some embodiments, the cancer is thyroid cancer; in some embodiments, the cancer is uterine sarcoma; in some embodiments, the cancer is vaginal cancer; in some embodiments, the cancer is vulvar cancer; in some embodiments, the cancer is Wilms tumor; in some embodiments, the cancer is laryngeal or hypopharyngeal cancer; in some embodiments, the cancer is kidney cancer; in some embodiments, the cancer is Kaposi sarcoma; in some embodiments, the cancer is gestational trophoblastic disease; in some embodiments, the cancer is gastrointestinal stromal tumor; in some embodiments, the cancer is gastrointestinal carcinoid tumor; in some embodiments, the cancer is gallbladder cancer; in some embodiments, the cancer is eye cancer (melanoma and lymphoma); in some embodiments, the cancer is Ewing tumor; in some embodiments, the cancer is esophagus cancer; in some embodiments, the cancer is endometrial cancer; in some embodiments, the cancer is colorectal cancer; in some embodiments, the cancer is cervical cancer; in some embodiments, the cancer is brain or spinal cord tumor; in some embodiments, the cancer is bone metastasis; in some embodiments, the cancer is bone cancer; in some embodiments, the cancer is bladder cancer; in some embodiments, the cancer is bile duct cancer; in some embodiments, the cancer is anal cancer; and in some embodiments, the cancer is adrenal cortical cancer.

In some embodiments, the disorder or disease is a neurological condition, disorder, or disease, wherein the neurological disease is selected from: Alzheimer's disease, frontotemporal dementias, Parkinson's disease, Huntington's disease, progressive supranuclear palsy, corticobasal degeneration, multiple system atrophy, amyotrophic lateral sclerosis (ALS), inclusion body myositis, autism, degenerative myopathies.

In some embodiments, the disorder or disease is selected from the group consisting of: Alzheimer's Disease, Amyotrophic Lateral Sclerosis, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, and Stroke.

In some embodiments, a compound of Formulas I, Ia, Ib, and Ic inhibits DYRK1A .

In some embodiments, the method treats a disease or disorder mediated by kinase activity in a patient, the method comprises administering to the patient a therapeutically effective amount of a compound (or compounds) of Formulas I, Ia, Ib, and Ic, or a pharmaceutically acceptable salt thereof.

In some embodiments, the disease or disorder comprises tumor growth, cell proliferation, or angiogenesis.

In some embodiments, the method inhibits the activity of a protein kinase receptor, the method comprises contacting the receptor with an effective amount of a compound (or compounds) of Formulas I, Ia, Ib, and Ic, or a pharmaceutically acceptable salt thereof.

In some embodiments, the method treats a disease or disorder associated with aberrant cellular proliferation in a patient; the method comprises administering to the patient a therapeutically effective amount of a compound (or compounds) of Formulas I, Ia, Ib, and Ic, or a pharmaceutically acceptable salt thereof.

In some embodiments, the method prevents or reduces abnormal cellular proliferation in a patient; the method comprises administering to the patient a therapeutically effective amount of a compound (or compounds) of Formulas I, Ia, Ib, and Ic, or a pharmaceutically acceptable salt thereof

In some embodiments, the method treats a disease or disorder associated with aberrant cellular proliferation in a patient, the method comprises administering to the patient a pharmaceutical composition comprising one or more of the compounds of claim 1 in combination with a pharmaceutically acceptable carrier and one or more other agents.

Evaluation of Biological Activity

The biological activity of the compounds described herein can be tested using any suitable assay known to those of skill in the art. For example, the activity of a compound may be tested using one or more of the test methods outlined below.

For example, in vitro assays for DYRK1A biological activity may be used, e.g., regulation of microtubule-associated protein tau (MAPT/Tau) phosphorylation in neuronal cell line such as the human SH-SY5Y neuroblastoma cell line. Assays for DYRK1A-regulated level of phosphorylation can include monitoring levels of basal pSer396 Tau, which can be measured, for example, by serial dilutions of a candidate inhibitor composition using a ten micromolar top concentration and detected by ELISA or Western Blotting. An exemplary assay for DYRK-1A-regulated phosphorylation uses the SH-SY5Y cells cultured in a 96 well plate format for a period of time sufficient to stabilize microtubules and Tau phosphorylation, usually at least 2 days, then treated with a 1/3 serial dilution of compounds overnight and lysed. The cell lysate is resolved by SDS PAGE, then transferred to nitrocellulose and probed with an antibody specific for pSer396 Tau. The chemiluminescence signal for HRP-linked antibodies used in western blotting is detected using a Carestream Image Station and blot densitometry for pSer396 and beta-actin are analyzed using ImageJ (NIH).

In a further example, the activity of a candidate compound can be measured by phosphoTau (Thr212) AlphaLISA by adding the lysate mentioned above onto total Tau-coated plates and detected with a specific pThr212Tau antibody. Colorimetric detection of AlphaLISA signal is performed by EnVision Multilabel Plate Reader (Perkin Elmer).

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described and claimed herein. The reader will recognize that the skilled artisan, armed with the present disclosure, and skill in the art is able to prepare and use the disclosure without exhaustive examples.

### EXAMPLES

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety)and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

(¹H) nuclear magnetic resonance spectra (NMR) were measured in the indicated solvents on a Bruker NMR spectrometer (Avance TM DRX300, 300 MHz for ¹H or Avance TM DRX500, 500 MHz for ¹I-1) or Varian NMR spectrometer (Mercury 400 BB, 400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; ddd, doublet of doublets of doublets; d/ABq, doublet of AB quartet; dt, doublet of triplets; td, triplet of doublets; dq, doublet of quartets; m, multiplet.

The following abbreviations have the indicated meanings:

Ac₂O=acetic anhydride

BrettPhos=2-(dicyclohexylphosphino)3,6-dimethoxy-2′,4′,6′-triisopropyl-1,1′-biphenyl

BrettPhos Pd G3=[(2-di-cyclohexylphosphino -3,6-dimethoxy-2′,4′,6′-triisopropyl-1,1′-biphenyl)-2-(2′-amino-1,1′-biphenyl)] palladium (II) methanesulfonate methanesulfonate brine=saturated aqueous sodium chloride

CDCl₃=deuterated chloroform

Cy₂PTipp=dicyclohexyl(2,4,6-triisopropylphenyl)phosphine

DCE=dichloroethane

DCM=dichloromethane

DIPEA=N,N-diisopropylethylamine

DMA=dimethylacetamide

DMAP=4-dimethylaminopyridine

DMF=N,N-dimethylformamide

DMPU=N,N′-dimethylpropyleneurea

DMSO-d6=deuterated dimethylsulfoxide

ESIMS=electron spray mass spectrometry

EtOAc=ethyl acetate

HATU=1-[bis(dimethylamino)methylene]-1H-1,2,3-triazolo[4,5-b]-blpyridinium 3-oxid hexafluorophosphate

HCl=hydrochloric acid

HOAc=acetic acid

IPA=isopropyl alcohol

ISCO=Teledyne ISCO, Inc brand CombiFlash® Rf 200

KOAc=potassium acetate

LAH=lithium aluminum hydride

Lawesson reagent=2,4-bis(4-methoxyphenyl)-1,3,2,4-dithiadiphosphetane-2,4-disulfide

LC/MS=liquid chromatography—mass spectrometry

LiN(SiMe₃)₂=lithium bis(trimethylsilyl)amide

MeCN=acetonitrile

MeOH=methanol

MgSO₄=magnesium sulfate

MsCl=mesyl chloride or methanesulfonyl chloride

MTBE=methyl tert-butyl ether

MW=microwave irradiation

NaBH₃CN=sodium cyanoborohydride

NaHCO₃=sodium bicarbonate

NaBH(OAc)₃=sodium triacetoxyborohydride

NIS=N-iodosuccinimide

NMR=nuclear magnetic resonance

ON=overnight

PCy₃=tricyclohexylphosphine

Pd₂(dba)₃=tris(dibenzylideneacetone)dipalladium(0)

Pd(dppf)Cl₂=1,1′-bis(diphenylphosphino)ferrocene-palladium(II)dichloride

Pd(PPh₃)₄=tetrakis(triphenylphosphine)palladium(0)

PE=petroleum ether

PPTS=pyridine p-toluenesulfonate

r.t.=room temperature

Selectfluor=1-chloromethyl-4-fluoro-1,4-diazoniabicyclo[2.2.2]octane

bis(tetrafluoroborate) (F-TEDA)

SEM-C1=(2-(chloromethoxy)ethyl)trimethylsilane

SPhos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate

SPhos Pd G4=methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II)

TBAF=tetra-n-butylammonium fluoride,

TEA=triethylamine

TFA=trifluoroacetic acid

THF=tetrahydrofuran

TLC=thin layer chromatography

TsCl=p-toluenesulfonyl chloride

Vilsmeier reagent=chloromethylene(dimethyl)iminium chloride

X-PHOS=2-dicyclohexylphosphino-2′,4′,6′-triisopropylbiphenyl

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only and should not be construed as or confused with same numberings in other sections of the application. Unless otherwise indicated, all variables are as defined above.

### General Procedures

Compounds of Formula Ic of the present disclosure can be prepared as depicted in Scheme 1.

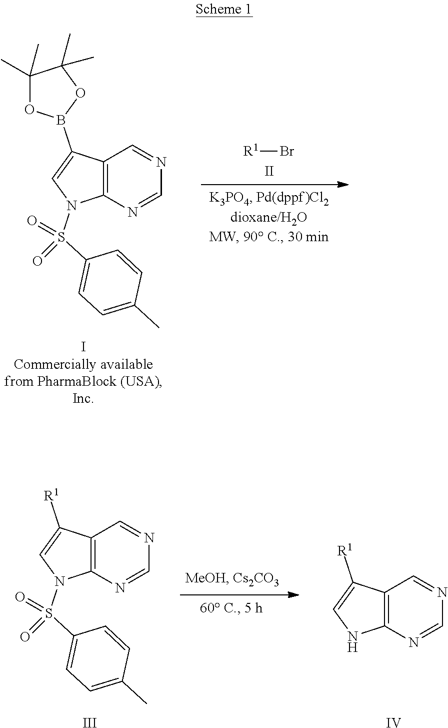

Scheme 1 describes a method for preparation of 7H-pyrrolo12,3-dlpyrimidine derivatives (IV) by first reacting the boronic acid pinacol ester (I) using Suzuki coupling with a variety of bromines (II) to produce protected 7H-pyrrolo[2,3-d]pyrimidine III. Removal of the tosyl sulfonyl protecting group with Cs₂CO₃ in MeOH produces the final R¹ substituted 7H-pyrrolo[2,3-d] pyrimidine IV.

In some embodiments, compounds of Formulas I and Ia of the present disclosure can be prepared as depicted in Scheme 2.

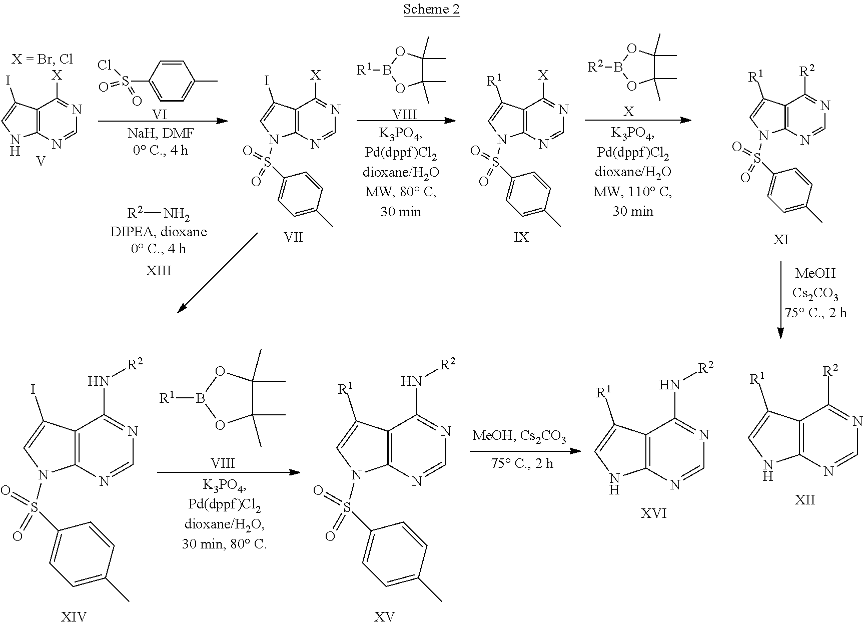

Scheme 2 describes a method for preparation of R² substituted 7H-pyrrolo[2,3-d]pyrimidine derivatives (XII) and (XVI) by first protecting the pyrrolo N of compound V with tosyl chloride (VI) to form VII. Suzuki coupling of the iodo (VII) with various boronic acid pinacol esters (VIII) yield derivatives IX. The halide on compound IX can be reacted with various boronic acid pinacol esters (X) produces protected compounds (XI) which after deprotection yields 1H-pyrrolo12,3-blpyridine derivatives (XII). Alternatively, the halide group X on VII can be displaced with an amine (XIII), followed by reaction of the iodo on compound VII can be coupled using Suzuki coupling with a variety of boronic acid pinacol esters (VIII) under Suzuki conditions to produce protected 7H-pyrrolo12,3-dlpyrimidine XV which after deprotection yields 7H-pyrrolo[2,3-d] pyrimidine derivatives (XVI).

In other embodiments, compounds of Formulas I and Ib of the present disclosure can be prepared as depicted in Scheme 3.

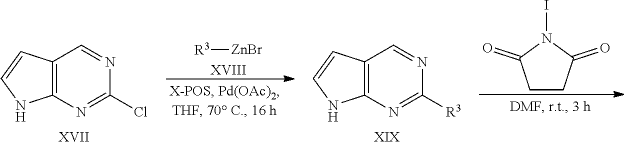

Scheme 3 describes a method for preparation of R³ substituted 7H-pyrrolo[2,3-d]pyrimidine derivatives (XXIII) by first using Negishi coupling of the chloride of XVII with a variety of organozinc compounds (XVIII) to produce derivative XIX. The R³ substituted 7H-pyrrolo12,3-dlpyrimidine (XIX) is iodinated with N-iodosuccinimide to produce compound XX. The pyrrolo N of compound XX is protected with tosyl chloride (VI) to form XXI. Suzuki coupling of the iodo (XXI) with various boronic acid pinacol esters (VIII) yields derivatives XXII which after deprotection yields 7H-pyrrolo12,3-dlpyrimidine derivatives (XXIII).

In other embodiments, compounds of Formulas I and Ib of the present disclosure can be prepared as depicted in Scheme 4.

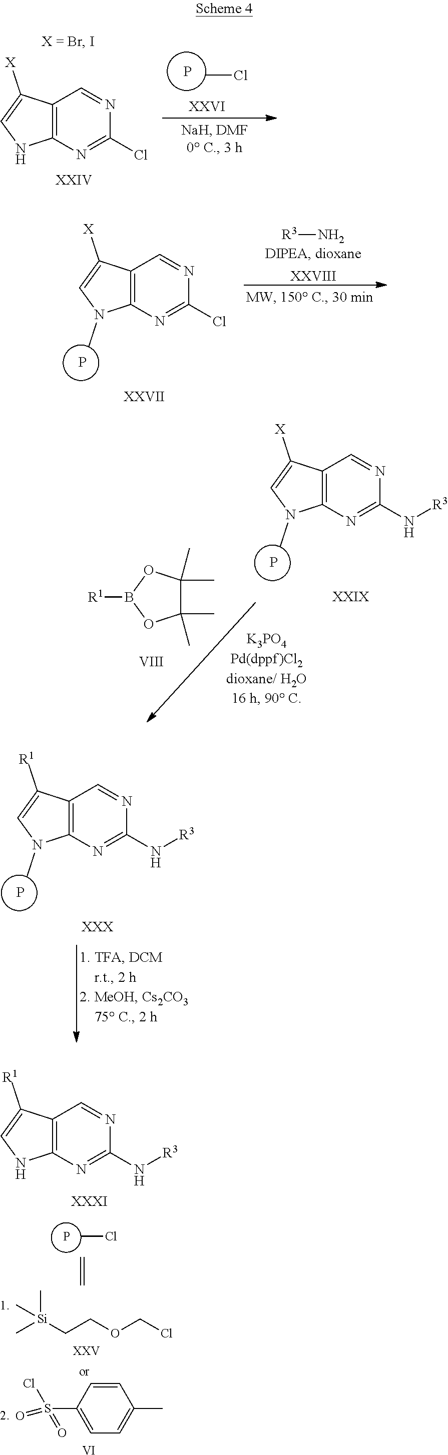

Scheme 4 describes a method for preparation of additional R³ substituted 7H-pyrrolo [2,3-d]pyrimidine derivatives (XXXI) by first protecting the pyrrolo N of compound V with tosyl chloride (VI) or SEM-Cl (XXV) to form XXVII. The chlorine of XXVII is then displaced with a variety of amines (XXVIII) to produce derivatives (XXIX). The remaining halide (X) is coupled with a variety of boronic acid pinacol esters (VIII) to give derivatives (XXX) which after deprotection yields R³ substituted 7H-pyrrolo[2,3-d]pyrimidine derivatives (XXXI).

### Illustrative Compound Examples

Preparation of intermediate 2-chloro-5-iodo-4-methoxy-7-tosyl-7H-pyrrolo[2,3-d]pyrimidine (XXXV) is depicted below in Scheme 5.

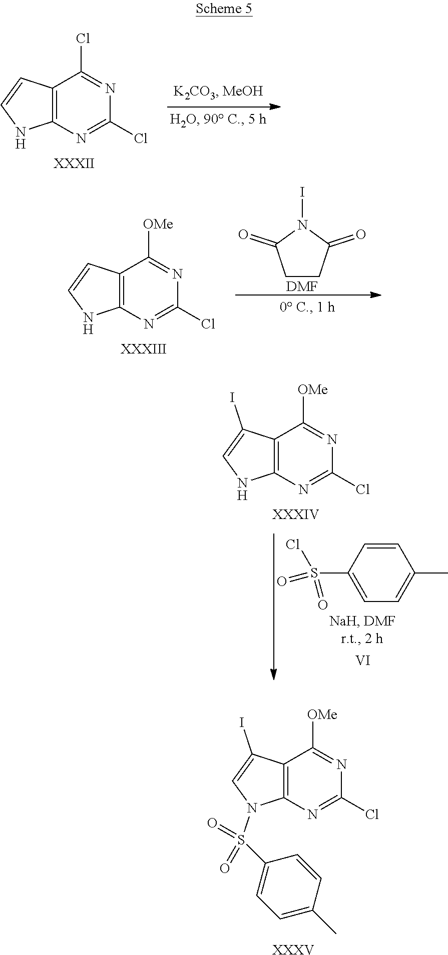

### Step 1

To a solution of 2,4-dichloro-7H-pyrrolo[2,3-d]pyrimidine (XXXII) (25 g, 0.13 mol), K₂CO₃ (36.8 g, 0.27 mol) in MeOH (250 mL) was added to H₂O (150 mL) at 25° C. The mixture was stirred at 90° C. for 5 h. After completion, the mixture was filtered to give 2-chloro-5-iodo-4-methoxy-7H-pyrrolo[2,3-d]pyrimidine (XXXIII) (23 g, 125.3 mmol, 96.4% yield) as a white solid. ESIMS found for C₇H₆ClN₃Om/z 184.1 (M+H).

### Step 2

To a solution of 2-chloro-5-iodo-4-methoxy-7H-pyrrolo[2,3-d]pyrimidine (XXXIII) (12.5 g, 0.06 mol) in DMF (130 mL) was added NIS (18.4 g, 0.08 mol) at 0° C. The mixture was stirred at 0° C. for 1 h. After completion, the mixture was quenched by sodium sulfite, diluted with H₂O (80 mL) and extracted with EtOAc (100 mL). The combined organic layers concentrated to produce 2-chloro-5-iodo-4-methoxy-7H-pyrrolo[2,3-d]pyrimidine (XXXIV) (15 g, 48.5 mmol, 80.8% yield) as a white solid. ESIMS found for C₇H₅ClIN₃O m/z 309.8 (M+H).

### Step 3

To a solution of 2-chloro-5-iodo-4-methoxy-7H-pyrrolo[2,3-d]pyrimidine (XXXIV) (5 g, 0.016 mmol) in DMF (250 mL) was added NaH (1.52 g, 0.323 mol) at 0° C. The mixture was stirred at 0° C. for 0.5 h. TsCl (IV) (6.16 mg, 0.323 mol) was added to the mixture at 0° C. and stirred for 2 h. The mixture was quenched by aqueous NH₄C1 and extracted with EtOAc (100 mL). The crude residue was purified by silica gel column (petroleum ether/EtOAc=20:1→5:1) to give 2-chloro-5-iodo-4-methoxy-7-tosyl-7H-pyrrolo[2,3-d]pyrimidine (XXXV) (5.8 g, 12.51 mmol, 78.2% yield) as a red solid. ESIMS found for C₁₄H₁₁ClN₃O₃S m/z 463.8 (M+H).

The following intermediates were prepared in accordance with the procedure described in the above Scheme 5.

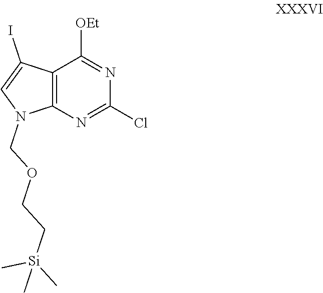 [SMILES: CCOc1nc(Cl)nc2c1c(I)cn2COCC[Si](C)(C)C]

SEM protection followed procedure in Example 2, Scheme 25, Step 1.

2-Chloro-4-ethoxy-5-iodo-7-42-(trimethylsilypethoxy)methyl)-7H-pyrrolo[2,3-d]pyrimidine (XXXVI): White solid (11.5 g, 25.34 mmol, 75.0% yield). 1H NMR (400 MHz, DMSO-d₆) δ-0.00 (d, J=3.3 Hz, 9H), 0.94-0.88 (m, 2H), 1.49 (t, J=7.1 Hz, 3H), 3.61-3.55 (m, 2H), 4.60 (q, J=7.0 Hz, 2H), 5.56 (s, 2H), 7.85 (s, 1H); ESIMS found for C₁₄H₂₁ClIN₃O₂Si m/z 454.0 (M+H).

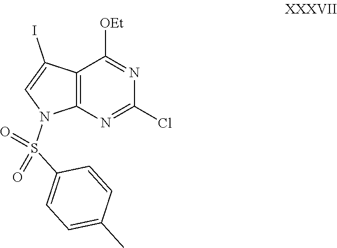 [SMILES: CCOc1nc(Cl)nc2c1c(I)cn2S(=O)(=O)c1ccc(C)cc1]

2-Chloro-4-ethoxy-5-iodo-7-tosyl-7H-pyrrolo[2,3-d]pyrimidine (XXXVII): White solid (2.56 g, 5.36 mmol, 91.7% yield). ESIMS found for CisH₁₃ClIN₃O₃S m/z 477.9 (M+H).

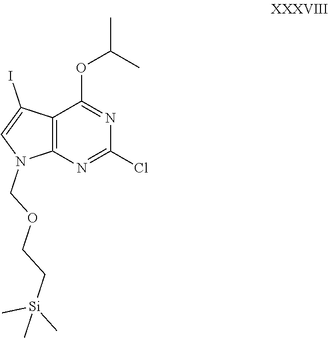 [SMILES: CC(C)Oc1nc(Cl)nc2c1c(I)cn2COCC[Si](C)(C)C]

SEM protection followed procedure in Example 2, Scheme 25, Step 1.

2-Chloro-5-iodo-4-isopropoxy-7-((2-(trimethylsilyl)ethoxy)methyl)-7H-pyrrolo[2,3-d]pyrimidine (XXXVIII): Colorless syrup (2.09 g, 4.468 mmol, 99.2% yield). ESIMS found for C ₁₅H₂₃ ClIN₃ 0₂ Si M/Z 468.0 (M+H).

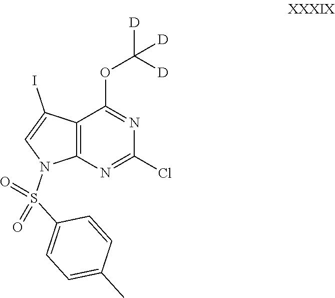 [SMILES: [2H]C([2H])([2H])Oc1nc(Cl)nc2c1c(I)cn2S(=O)(=O)c1ccc(C)cc1]

2-Chloro-5-iodo-4-(methoxy-d₃)-7-tosyl-7H-pyrrolo [2,3-d] pyrimidine (XXXIX): White solid (2.0 g, 4.292 mmol). ¹H NMR (400 MHz, DMSO-d₆) δppm 2.37 (3 H, s), 7.48 (2 H, d, J=7.95 Hz), 8.00 (2 H, br d, J=8.44 Hz), 8.00 (1 H, s); ESIMS found for C₁₄H₈D₃ClIN₃O₃S m/z 466.9 (M+H).

Preparation of intermediate 5-bromo-2-chloro-N-methyl-7-tosyl-7H-pyrrolo[2,3-d]pyrimidin-4-amine (XLII) is depicted below in Scheme 6.

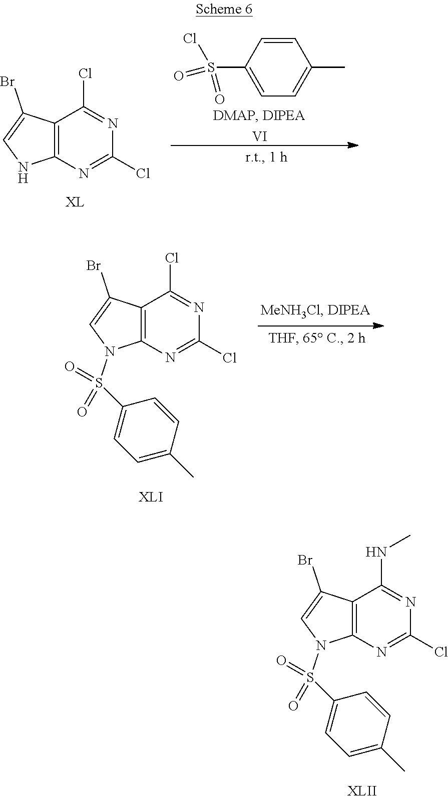

### Step 1

5-bromo-2,4-dichloro-7H-pyrrolo [2,3-d] pyrimidine (XL) (commercially available from Synthonix, Inc.) (1.0 g, 3.75 mmol) and DMAP (45.7 mg, 0.37 mmol) were dissolved in dry DCM (30 mL) before adding DIPEA (1.3 mL, 7.47 mmol). The reaction was stirred for 5 min. Tosyl chloride (VI) (1.07 g, 5.62 mmol) was added and the reaction was stirred at room temperature for 1 h. The reaction mixture was poured into 1 M HC1, and the organic layer was separated. The aqueous layer was extracted with DCM (x3) and the combined organic layers were washed with brine, dried (anhydrous MgSO₄) and reduced in vacuo to give the crude product as a brown solid. The crude product was purified by column chromatography (0→25% EtOAc/hexanes). Appropriate fractions were collected and reduced in vacuo to give an off-white solid. The solid was triturated in MeOH and filtered, washing with MeOH. The filtrate was reduced in vacuo and triturated as before. Both batches were combined to give 5-bromo-2,4-dichloro-7-(4-methylphenyl)sulfonylpyrrolo [2,3-d]pyrimidine (XLI) (1.356 g, 3.220 mmol, 85.9% yield) as a white solid. ESIMS found for C 13H₈BrC12N3 02 S mlz 419.9 (M+H).

### Step 2

5-bromo-2,4-dichloro-7-(4-methylphenyl)sulfonylpyrrolo[2,3-d]pyrimidine (XLI) (1.0 g, 2.37 mmol) and methylammonium chloride (403 mg, 5.97 mmol) were suspended in dry THF (15 mL) and then cooled to -78° C. DIPEA (1.5 mL, 8.61 mmol) was added, and the reaction was stirred at 65° C. for 2 h. The reaction mixture was added to 0.5 M HC1, and the organic layer was separated. The aqueous layer was extracted with DCM (x3) and the combined organic layers were washed with brine, dried (anhydrous MgSO₄) and reduced in vacuo to give the crude product as a yellow solid. The crude product was purified by column chromatography (0-45% EtOAc/hexanes). Appropriate fractions were collected and reduced in vacuo to give an off-white solid. The product was triturated in MeOH and filtered, washing with MeOH to yield 5-bromo-2-chloro-N-methyl-7-(4-methylphenyl)sulfonylpyrrolo [2,3-d]pyrimidin-4-amine (XLII) (534 mg, 1.285 mmol, 54.1% yield) as a white solid. ESIMS found for C₁₄H₁₂BrClN4O₂S m/z 415.0 (M+H).

Preparation of intermediate 6-bromo-3-(difluoromethyl)imidazo [1,2-a]pyridine (XLV) is depicted below in Scheme 7.

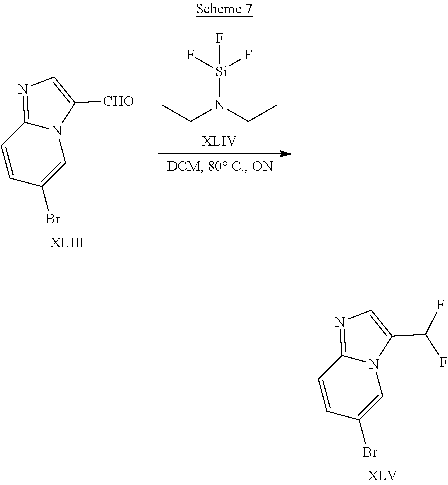

### Step 1

To a stirred solution of 6-bromoimidazo[1,2-a]pyridine-3-carbaldehyde (XLIII) (commercially available from Combi-Blocks Inc.) (500 mg, 2.22 mmol) in DCM (8 mL) was added drop wise N,N-diethyl-1,1,1-trifluorosilanamine (XLIV) (0.56 mL, 4.54 mmol) at room temperature. Then, the reaction mixture was heated to 80° C. overnight. LCMS showed 50% completion of the reaction. The reaction mixture was continued to stir at the same temperature additional 6 h without any additional conversion. The reaction mixture was cooled to room temperature, diluted with DCM, washed with aqueous saturated NaHCO₃, brine, and dried over anhydrous Na2SO₄. The organic phase was concentrated under high vacuum, and the residue was purified by ISCO (10→80% EtOAc/hexanes) to obtain 6-bromo-3-(difluoromethyl)imidazo [1,2]-alpyridine (XLV) (201 mg, 0.814 mmol, 36.6% yield) as a beige solid. ESIMS found for C₈H₅BrF₂N₂ m/z 246.95 (⁷⁹BrM+H).

The following intermediates were prepared in accordance with the procedure described in the above Scheme 7.

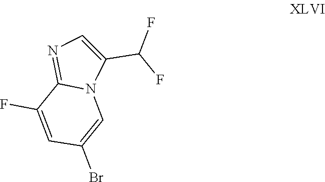 [SMILES: Fc1cc(Br)cn2c(C(F)F)cnc12]

6-Bromo-3-(difluoromethyl)-8-fluoroimidazo[1,2-a]pyridine (XLVI): Off-white (125 mg, 0.472 mmol, 45.9% yield). ESIMS found for C₈H₄BrF₃N₂ m/z 264.95 (M+H).

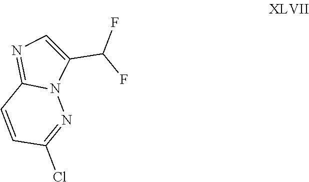 [SMILES: FC(F)c1cnc2ccc(Cl)nn12]

6-Chloro-3-(difluoromethyl)imidazo[1,2-b]pyridazine (XLVII): Off-white (125 mg, 0.472 mmol, 45.9% yield). ¹H NMR (400 MHz, DMSO-d₆) δppm 7.68-7.37 (m, 2H), 8.16 (t, J=1.7 Hz, 1H), 8.37 (d, J=9.6 Hz, 1H); ESIMS found for C₇H₄ClF₂N₃ m/z 204.1 (M+H).

Preparation of intermediate 5-bromo-3-(1-methyl-1H-pyrazol-4-yl)pyrazolo[1,5-a]pyridine (L) is depicted below in Scheme 8.

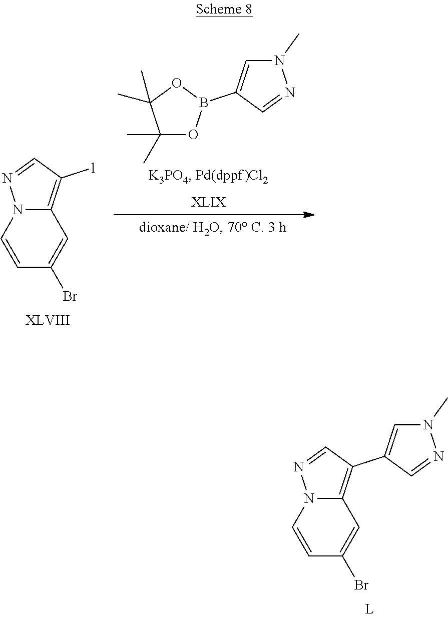

### Step 1

A mixture of 1-methyl-4-(4,4,5,5-tetramethyl-1,3,2-dioxaborolan-2-yl)pyrazole (XLIX) (0.15g, 0.7200 mmol) , 5-bromo-3-iodopyrazolo[1,5-a]pyridine (XLVIII) (0.22 g, 0.670 mmol), was dissolved in 1,4-dioxane (3 mL) before adding Pd(dppf)Cl₂ (55 mg, 0.067 mmol) and 2 M aqueous solution of K3PO₄ (0.75 mL, 1.5 mmol). N₂ gas was bubbled into the mixture for 5 min and then mixture was heated to 70° C. for 3 h. LCMS showed both the desired product and the double Suzuki product (3:2). The organic layer was separated, absorbed on silica gel and purified by ISCO (0→100% EtOAc/hexanes) to obtain the desired product 5-bromo-3-(1-methylpyrazol-4-yl)pyrazolo[1,5-a]pyridine (L) (80 mg, 0.289 mmol, 43.4% yield) as a pale-yellow solid. ESIMS found for C₁₁H₉BrN4 m/z 276.95 (M+H).

Preparation of intermediate 6-bromo-8-fluoro-2-methylimidazo[1,2-a]pyridine (LIII) is depicted below in Scheme 9.

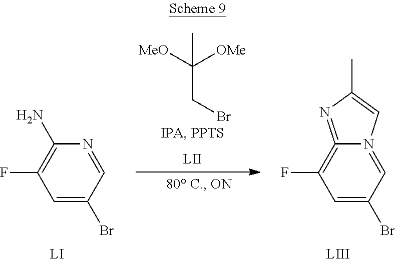

### Step 1

A mixture of 5-bromo-3-fluoropyridin-2-amine (LI) (2.g, 10.47 mmol), 1-bromo-2,2-dimethoxypropane (LII) (2.11g, 11.53 mmol) and PPTS (0.26 g, 1.05 mmol) in IPA (25 mL) was heated to 80° C. overnight. The reaction mixture was cooled to room temperature, added to water (200 mL) and stirred for 1 h. The resulting solids were collected and dried under high vacuo to obtain 6-bromo-8-fluoro-2-methylimidazo[1,2-a]pyridine (LIII) (2.61 g, 11.40 mmol, 108.8% yield) as beige solid which was used for the next step without further purification. ESIMS found for C₈H₆BrFN₂ m/z 229.0 (M+H).

The following intermediate was prepared in accordance with the procedure described in the above Scheme 9.

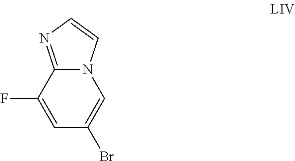 [SMILES: Fc1cc(Br)cn2ccnc12]

6-Bromo-8-fluoroimidazo[1,2-a]pyridine (LIV): Beige solid (909.0 mg, 4.228 mmol, 80.7% yield). ESIMS found for C7H₄BrFN2 m/z 214.9 (M+H).

Preparation of intermediate 6-bromo-8-fluoroimidazo[1,2-a]pyridine-3-carbaldehyde (LVI) is depicted below in Scheme 10.

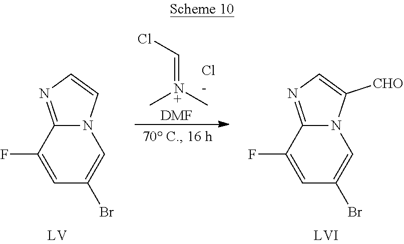

### Step 1

To a stirred suspension of the Vilsmeier reagent (0.72 g, 5.59 mmol) in DMF (2 mL) was added 6-bromo-8-fluoroimidazo[1,2-a]pyridine (LV) (0.4 g, 1.86 mmol) in DMF (4 mL). The mixture was heated to 70° C. for 16 h. The reaction mixture was cooled, added to ice, basified with 2 N NaOH, stirred for 1 h and the solids were collected by filtration. The solids were taken in CHCl₃, washed with brine solution, dried over anhydrous Na2SO₄, evaporated, and dried under high vacuo to obtain 6-bromo-8-fluoroimidazo[1,2-a]pyridine-3-carbaldehyde (LVI) (360.0 mg, 1.481 mmol, 79.6% yield) as a beige solid. ESIMS found for CsH₄BrFN₂O m/z 243.1 (M+H).

Preparation of intermediate 6-bromo-3-fluoroimidazo[1,2-a]pyridine (LVIII) is depicted below in Scheme 11.

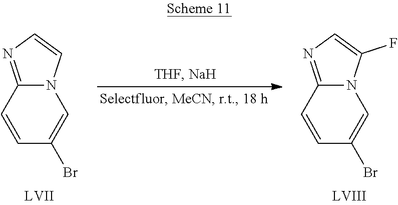 [SMILES: Brc1ccc2nccn2c1 ; Fc1cnc2ccc(Br)cn12]

### Step 1

To a solution of 6-bromoimidazo[1,2-a]pyridine (LVII) (0.5 g, 2.54 mmol) in THF (20 mL) at 0° C. was added NaH (0.15 g, 3.82 mmol). The reaction mixture was stirred at room temperature for 30 min. MeCN (10 mL) was added followed by the addition of Selectfluor (1.35 g, 3.81 mmol) and the reaction mixture was stirred at room temperature for 18 h. The reaction mixture was absorbed on silica gel, purified by ISCO (0-400% EtOAc/hexanes) to obtain 6-bromo-3-fluoroimidazo[1,2-a]pyridine (LVIII) (110 mg, 0.512 mmol, 20.2% yield) as beige solid. ESIMS found for C₇H₆BrClN₂O m/z 250.9 (M+H).

The following intermediate was prepared in accordance with the procedure described in the above Scheme 11.

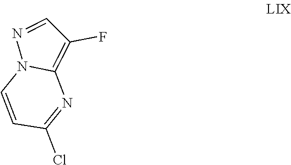 [SMILES: Fc1cnn2ccc(Cl)nc12]

5-Chloro-3-fluoropyrazolo [1,5-a] pyrimidine (LIX): Light-yellow solid (985.0 mg, 3.633 mmol, 57.2% yield). ¹1-1 NMR (400 MHz, DMSO-d₆) δppm 7.15 (d, J=7.2 Hz, 1H), 8.44 (d, J=3.6 Hz, 1H), 9.12-9.09 (m, 1H); ESIMS found for C₆H₃ClFN₃ m/z 272.0 (M+H).

Preparation of intermediate 5-chloro-3-cyclopropylpyrazolo[1,5-a]pyrimidine (LXIII) is depicted below in Scheme 12.

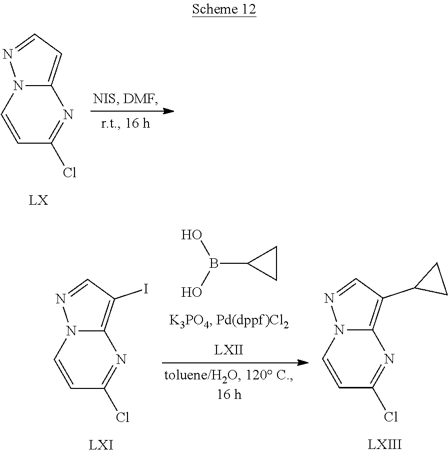 [SMILES: Clc1ccn2nccc2n1 ; Clc1ccn2ncc(I)c2n1 ; OB(O)C1CC1 ; Clc1ccn2ncc(C3CC3)c2n1]

### Step 1

To a solution of 5-chloropyrazolo[1,5-a]pyrimidine (LX) (3.0 g, 19.54 mmol) in DMF (30.0 mL) was added NIS (4.83 g, 21.49 mmol) at 0° C. The mixture was stirred at room temperature for 16 h. The reaction solution was diluted with water (100 mL) and extracted with EtOAc (2×200 mL). The combined organic layers were dried with Na₂SO₄, filtered, and concentrated. The crude product was purified by silica gel column chromatography (50% EtOAc/petroleum ether) to obtain 5-chloro-3-iodopyrazolo[1,5-a]pyrimidine (LXI) (4.5 g, 16.13 mmol, 82.5% yield) as a light-yellow solid. ¹H NMR (400 MHz, DMSO-d₆) δppm 6.83 (d, J=6.6 Hz, 1H), 8.14 (s, 1H), 8.55 (d, J=7.2 Hz, 1H); ESIMS found for C₆H₃ClIN₃m/z 279.8 (M+H).

### Step 2

To a solution of 5-chloro-3-iodopyrazolo [1,5-a]pyrimidine (LXI) (4.6 g, 16.49 mmol) and cyclopropylboronic acid (LXII) (1.56 g, 18.14 mmol) in toluene/H₂O (40 mL/5 mL) was added K3PO₄ (7.0 g, 32.99 mmol) and Pd(dppf)C12 (0.27 g, 0.33 mmol) at room temperature, the mixture was stirred at 100° C. for 16 h under nitrogen. The reaction solution was filtered and extracted with EtOAc (150 mL). The combined organic layers were dried with Na2SO₄, filtered, and concentrated. The crude product was purified by column chromatographed (petroleum ether/EtOAc=3/7) to obtain 5-chloro-3-cyclopropylpyrazolo[1,5-a]pyrimidine (LXIII) (1.4 g, 7.25 mmol, 44.0% yield) as a light-yellow solid. ESIMS found for C₉H₈ClN₃ m/z 194.1 (M+H).

Preparation of intermediate imidazo[1,2-a]pyrimidin-6-ylboronic acid (LXVI) is depicted below in Scheme 13.

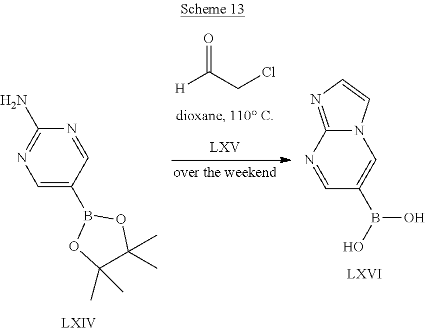

### Step 1

A mixture of 5-(4,4,5,5-tetramethyl-1,3 ,2-dioxaborolan-2-yl)pyrimidin-2-amine (LXIV) (1 g, 4.52 mmol) and chloroacetaldehyde (LXV) (0.92 mL, 5.39 mmol) was dissolved in 1,4-dioxane (20 mL) and heated to 110° C. over the weekend. The reaction mixture was cooled, and the solids were collected by filtration and dried under high vacuo to obtain imidazo [1,2-a]pyrimidin-6-ylboronic acid (LXVI) (650 mg, 3.989 mmol, 88.2% yield) as a brown solid which was used for next step without purification. ESIMS found for C₆H₆BN₃O₂ m/z 164.1 (M+H).

Preparation of intermediate 5-chloro-2-methylthiazolo [5,4-b]pyridine (LXIX) is depicted below in Scheme 14.

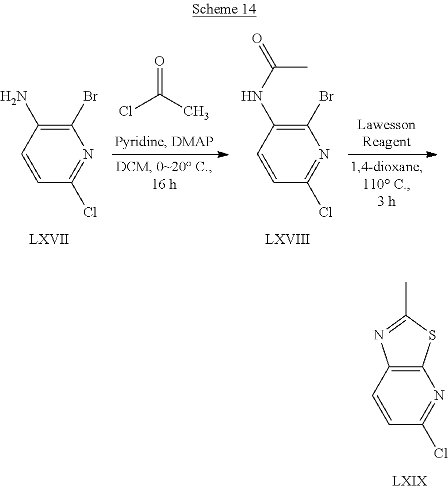

### Step 1

2-Bromo-6-chloropyridin-3-amine (LXVII) (1 g, 4.82 mmol), acetyl chloride (567.6 mg, 7.23 mmol), pyridine (762.5 mg, 9.64 mmol) and DMAP (58.9 mg, 0.482 mmol) were dissolved successively in DCM (10 mL) at 0° C. The resulting mixture was then stirred while allowing to warm from 0 to 20° C. over 16 h. The reaction mixture was quenched by stirring for 15 min with saturated aqueous NH₄C1. Subsequently, the mixture was diluted with EtOAc (30 mL). The organic layer was washed with water (30 mL), and the aqueous portion was extracted with EtOAc (30 mL). The combined organic extracts were washed with brine (30 mL) and dried over MgSO₄, filtrated, and concentrated in vacuo to afford the crude which was purified by silica gel column chromatography to afford the desired product N-(2-bromo-6-chloropyridin-3-yl)acetamide (LXVIII) (994 mg, 4.01 mmol, 83.2% yield) as a yellow solid. ESIMS found for C7H₆BrClN₂O m/z 250.9 (M+H).

### Step 2

N-(2-bromo-6-chloropyridin-3-yl)acetamide (LXVIII) (900 mg, 3.63 mmol) and Lawesson reagent (2.21 g, 5.45 mmol) were dissolved in dioxane (8 mL). The resulting mixture was stirred at 110° C. for 3 h. After the completion, the mixture was quenched by stirring for 15 min with saturated aqueous NH₄C1. The mixture was then diluted with EtOAc (30 mL). The solution was washed with water (30 mL), and the aqueous portion was extracted with EtOAc (3×30 mL). The combined organic extracts were washed with brine (3×30 mL) and dried over Na2SO₄, filtrated, and concentrated in vacuo to afford the crude which was purified by silica gel column chromatography to afford the desired product 5-chloro-2-methylthiazolo[5,4-b]pyridine (LXIX) (600 mg, 3.26 mmol, 89.8% yield) as a yellow solid. ¹H NMR (400 MHz, DMSO-d₆) δppm 2.85 (s, 3H), 7.64 (d, J=8.6 Hz, 1H), 8.36 (d, J=8.6 Hz, 1H); ESIMS found for C₇H₅ClN₂S m/z 185.0 (M+H).

Preparation of intermediate 6-bromo-8-fluoro-3-(1-methylpiperidin-4-yl)imidazo[1,2-a]pyridine (LXXII) is depicted below in Scheme 15.

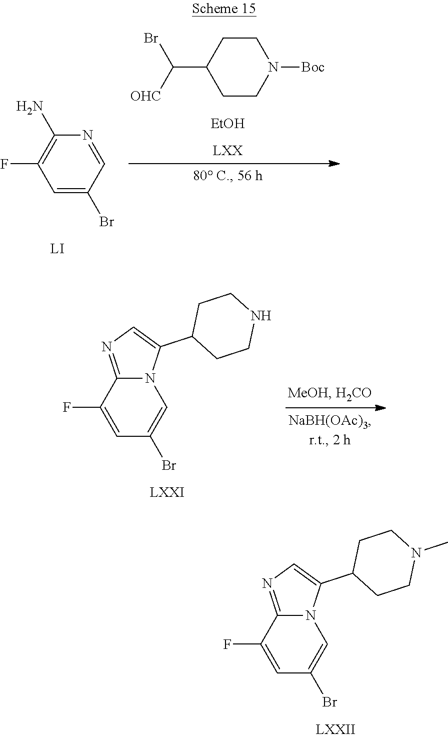 [SMILES: "EtOH" ; CC(C)(C)OC(=O)N1CCC(C(Br)C=O)CC1 ; Nc1ncc(Br)cc1F ; Fc1cc(Br)cn2c(C3CCNCC3)cnc12 ; CN1CCC(c2cnc3c(F)cc(Br)cn23)CC1]

### Step 1

A mixture of 5-bromo-3-fluoropyridin-2-amine (LI) (0.2 g, 1.05 mmol) and teat-butyl 4-(1-bromo-2-oxoethyl)piperidine-1-carboxylate (LXX) (0.48 g, 1.57 mmol) in EtOH (5 mL) was heated to 80° C. for 56 h. The reaction mixture was cooled to room temperature, concentrated under vacuum and the residue taken in DCM (3 mL) and TFA (1 mL). The reaction mixture was stirred for 30 min, concentrated and the residue purified by ISCO (1→10% 7 N NH₃ MeOH/CHCl₃) to obtain 6-bromo-8-fluoro-3-piperidin-4-ylimidazo[1,2-a]pyridine (LXXI) (286.0 mg, 0.959 mmol, 91.7% yield) as off-white solid. ESIMS found for C₁₂H₁₃BrFN₃m/z 298.0 (M+H).

### Step 2

To a stirred solution of 6-bromo-8-fluoro-3-piperidin-4-ylimidazo [1,2-a]pyridine (LXXI) (0.29 g, 0.960 mmol) in MeOH (4 mL) was added formaldehyde (0.12 mL, 1.59 mmol) and stirred for 5 min. NaBH(OAc)₃ (0.31g, 1.45 mmol) was then added and the mixture was stirred for at room temperature overnight. Additional equivalents of formaldehyde and borohydride were added, and the mixture was stirred for another 4 h. The solvent was concentrated, and the residue was purified by ISCO (1-40% 7 N NH₃ MeOH/CHCl₃). The purified fractions were concentrated, the residue suspended in EtOAc, insoluble solids were removed by filtration and the filtrates were concentrated to obtain 6-bromo-8-fluoro-3-(1-methylpiperidin-4-yl)imidazo [1,2-a]pyridine (LXXII) (155 mg, 0.497 mmol, 51.8% yield) as off-white gummy solids which is not pure and used as is for the next step. ESIMS found for C₁₃H₁₅BrFN₃ m/z 312.1 (M+H).

Preparation of intermediate 6-bromo-4-fluoro-2-methyl-1-(1-methylpiperidin-4-yl)-3a ,7a-dihydro-1H-benzo[d]imidazole (LXXVIII) is depicted below in Scheme 16.

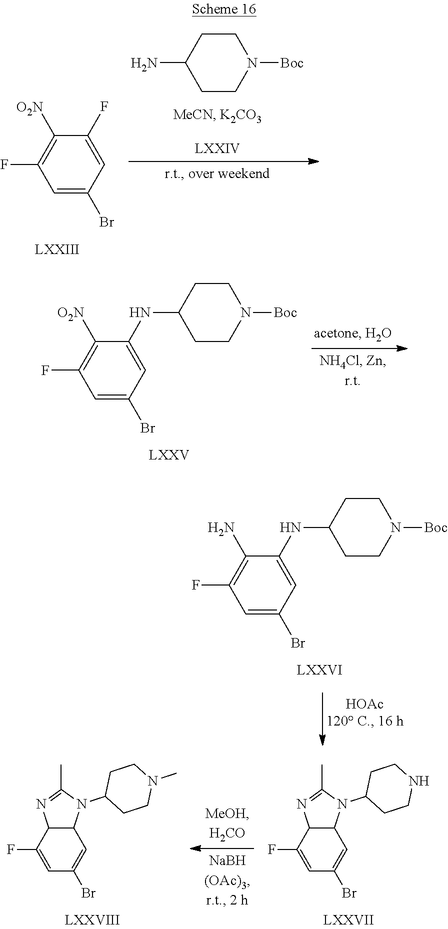 [SMILES: CC(C)(C)OC(=O)N1CCC(N)CC1 ; O=[N+]([O-])c1c(F)cc(Br)cc1F ; CC(C)(C)OC(=O)N1CCC(Nc2cc(Br)cc(F)c2[N+](=O)[O-])CC1 ; CC(C)(C)OC(=O)N1CCC(Nc2cc(Br)cc(F)c2N)CC1 ; CC1=NC2C(F)=CC(Br)=CC2N1C1CCN(C)CC1 ; CC1=NC2C(F)=CC(Br)=CC2N1C1CCNCC1]

### Step 1

A mixture of tent-butyl 4-aminopiperidine-1-carboxylate (LXXIV) (1.68 g, 8.4 mmol), 5-bromo-1,3-difluoro-2-nitrobenzene (LXXIII) (2 g, 8.4 mmol) and K2CO₃ (2.32 g, 16.81 mmol) in MeCN (40 mL) was stirred at room temperature over the weekend. The solvents were concentrated, and the residue partitioned between EtOAc/water. The organics were separated, washed with water and brine. The organics were dried over anhydrous Na2SO₄, solvents removed in vacuo and the crude was purified by ISCO (0→30% EtOAc/hexanes) to obtain teat-butyl 4-(5-bromo-3-fluoro-2-nitroanilino)piperidine-1-carboxylate (LXXV) (2.564 g, 6.130 mmol, 72.9% yield) as a yellow solid. ESIMS found for C₁₆H₂ᵢBrFN₃O₄m/z 362.00 (M+1-1-13u).

### Step 2

To a solution of tent-butyl 4-(5-bromo-3-fluoro-2-nitroanilino)piperidine-1-carboxylate (LXXV) (2.6 g, 6.22 mmol) and NH₄C1 (4.99 g, 93.24 mmol) in a mixture of acetone (75 mL) and water (15 mL) was added zinc powder (4.06 g, 62.16 mmol)(three equal portions over 5 minutes) at 0 ° C. The mixture was warmed to room temperature and then heated to 70° C. for 4 h. The mixture was filtered through ^{Celite}® and the solvents were concentrated under reduced pressure. The mixture was re-dissolved in EtOAc and filtered a second time through Celite® and the filtrates were washed with water, brine, dried over anhydrous Na₂SO₄, filtered, and concentrated under reduced pressure to obtain tent-butyl 4-(2-amino-5-bromo-3-fluoroanilino)piperidine-1-carboxylate (LXXVI) (2.41 g, 6.207 mmol, 99.8% yield) as a light brown solid. The resultant residue was used in the next reaction without further purification. ESIMS found for C₁₆H₂₃BrFN₃O₂ m/z 332.0 (M+H-^{t}Bu).

### Step 3

A solution of tent-butyl 4-(2-amino-5-bromo-3-fluoroanilino)piperidine-1-carboxylate (LXXVI) (900 mg, 2.32 mmol) in HOAc (12 mL) was heated to 120° C. for 16 h. The reaction mixture was concentrated, the residue treated with 7 N NH₃/MeOH, absorbed on silica and was purified by ISCO (100% CHCl₃-* 80% CHCl₃/10% 7 N NH₃ MeOH/CHCl₃) to obtain 6-bromo-4-fluoro-2-methyl-1-piperidin-4-ylbenzimidazole (LXXVII) (510 mg, 1.634 mmol, 70.5% yield) as a brown solid. ESIMS found for C₁₃H₁₇BrFN₃ m/z 314.0 (M+H).

### Step 4

To a stirred solution of 6-bromo-4-fluoro-2-methyl-1-piperidin-4-ylbenzimidazole (LXXVII) (250 mg, 0.800 mmol) in MeOH (4 mL) was added formaldehyde (0.08 mL, 2.9 mmol). After 15 min, NaBH(OAc)₃ (255 mg, 1.2 mmol) was added, and the mixture was stirred at room temperature 2 h. The solvents were concentrated, the residue taken in chloroform, washed with 1 N NaOH, water and brine. The organics were dried over anhydrous Na2SO₄, solvents removed and dried under high vacuo to obtain crude 6-bromo-4-fluoro-2-methyl-1-(1-methylpiperidin-4-yl)benzimidazole (LXXVIII) (237 mg, 0.727 mmol, 90.7% yield) as a light brown solid which was used for next step without purification. ESIMS found for C₁₄H₁₉BrFN3 m/z 328.05 (M+H).

Preparation of intermediate 6-bromo-1-isopropyl-2-methyl-1H-imidazo[4,5-b]pyridine (LXXXIII) is depicted below in Scheme 17.

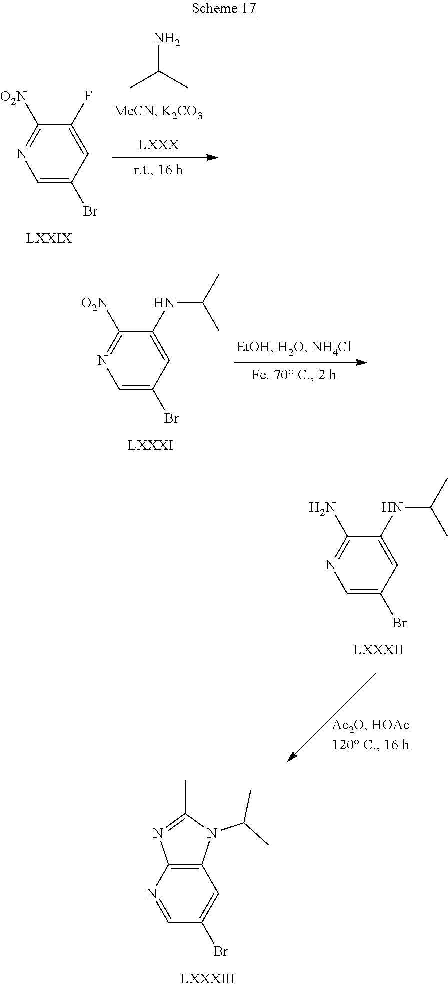

### Step 1

A mixture of 2-aminopropane (LXXX) (0.86 mL, 9.96 mmol), 5-bromo-3-fluoro-2-nitropyridine (LXXIX) (2 g, 9.05 mmol) and K2CO₃ (2.5 g, 18.1 mmol) in MeCN (40 mL) was stirred at room temperature for 16 h. The reaction mixture was added to water (200 mL), stirred for 1 h and the resulting solids were collected by filtration and dried under high vacuo to obtain 5-bromo-N-isopropyl-2-nitropyridin-3-amine (LXXXI) (2.36 g, 9.074 mmol, 100.3% yield) as a yellow solid which was used for next step without purification. ESIMS found for CalioBrN₃O₂ m/z 260.0 (M+H).

### Step 2

A mixture of 5-bromo-N-isopropyl-2-nitropyridin-3-amine (LXXXI) (2.35 g, 9.04 mmol) Fe (5.91 g, 90.35 mmol) and NH₄C1 (7.25 g, 135.53 mmol) was taken in a mixture of EtOH (30 mL), and water (10 mL) and the mixture was heated to 70° C. for 2 h. The reaction mixture was cooled, filtered through Celite , filtrates were taken into EtOAc, washed with water then brine, dried over anhydrous Na2SO₄, filtered, and concentrated under reduced pressure to obtain 5-bromo-N³-isopropylpyridine-2,3-diamine (LXXXII) (2.2 g, 9.561 mmol, 105.8% yield) as a dark brown solid which was used for next step without purification. ESIMS found for Cali₂BrN₃m/z 230.05 (M+H).

### Step 3

A solution of 5-bromo-N³-isopropylpyridine-2,3-diamine (LXXXII) (2.08 g, 9.04 mmol) and Ac2O (1.05 mL, 10.84 mmol) in HOAc (20 mL) was heated to 120° C. for 16 h. The reaction mixture was concentrated, the residue partitioned between EtOAc/1 N NaOH, organics separated, washed with water and brine. The organics were dried over anhydrous Na2SO₄, solvents concentrated and dried under high vacuo to give 6-bromo-1-isopropyl-2-methyl-1H-imidazo[4,5-b]pyridine (LXXXIII) (1.57 g, 6.178 mmol, 68.3% yield) as a dark brown solid which was used for next step without purification. ESIMS found for C₁₀H₁₂BrN₃ m/z 254.0 (M+H).

Preparation of intermediate 6-bromo-1-(2,2-difluoroethyl)-2-methyl-1H-imidazo[4,5-b]pyridine (LXXXVII) is depicted below in Scheme 18.

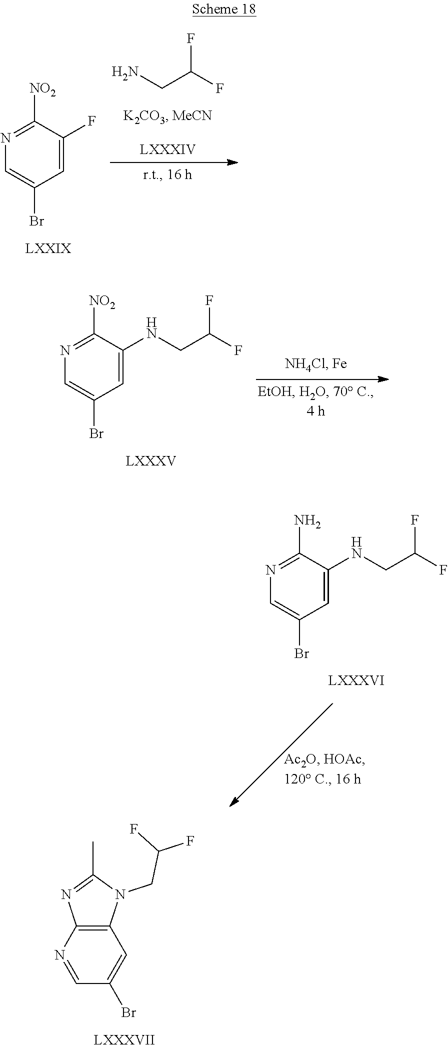

### Step 1

A mixture of 2,2-difluoroethan-1-amine (LXXXIV) (410 mg, 5.02 mmol), 5-bromo-3-fluoro-2-nitropyridine (LXXIX) (commercially available from Ark Pharma Scientific Limited) (1.0 g, 4.53 mmol) and K2CO₃ (1.38 g, 9.95 mmol) in MeCN (20 mL) was stirred at room temperature for 16 h. The reaction was filtered and concentrated under high vacuum. The residue was taken up in water, stirred for 1 hour and the solids were collected by filtration and dried in vacuo to obtain 5-bromo-N-(2,2-difluoroethyl)-2-nitropyridin-3-amine (LXXXV) (1.066 g, 3.780 mmol, 83.5% yield) as a yellow solid which was used for next step without purification. ESIMS found for C7H₆BrF2N3O₂ MiZ 282.0 (⁷⁹BrM+H).

### Step 2

A mixture of 5-bromo-N-(2,2-difluoroethyl)-2-nitropyridin-3-amine (LXXXV) (1.32 g, 4.69 mmol), Fe (3.07 g, 46.95 mmol) and NH₄C1 (3.77 g, 70.48 mmol) was taken in a mixture of EtOH (18 mL), and water (6 mL) and the mixture was heated to 70° C. for 4 h. The reaction mixture was cooled and filtered through Celite . The filtrates were taken up in EtOAc, washed with water and brine, dried over anhydrous Na2SO₄, filtered, and concentrated under reduced pressure to obtain 5-bromo-N³-(2,2-difluoroethyl)pyridine-2,3-diamine (LXXXVI) (630 mg, 2.499 mmol, 53.2% yield) as a grey solid which was used for next reaction without further purification. ESIMS found for C₇H₈BrF₂N₃m/z 252.0 (⁷⁹BrM+H).

### Step 3

A solution of 5-bromo-N³-(2,2-difluoroethyl)pyridine-2,3-diamine (LXXXVI) (630 mg, 2.5 mmol) and acetic anhydride (0.28 mL, 2.97 mmol) in HOAc (15 mL) was heated to 120° C. for 16 h. The reaction mixture was concentrated, the residue partitioned between Et0Aci1 N NaOH, organics separated, and washed with water and brine. The organics were dried over anhydrous Na₂SO₄, solvents and concentrated under high vacuum. The residue was triturated with diethyl ether, sonicated and the solids were collected by filtration and dried under high vacuo to obtain 6-bromo-1-(2,2-difluoroethyl)-2-methylimidazo[4,5-b]pyridine (LXXXVII) (325 mg, 1.177 mmol, 47.1% yield) as a grey solid which was used for next step without purification. ESIMS found for C₉H₈BrF₂N₃ m/z 276.0 (⁷⁹BrM+H).

The following intermediates were prepared in accordance with the procedure described in the above Scheme 18.

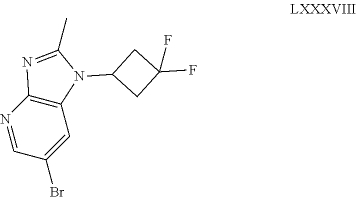 [SMILES: Cc1nc2ncc(Br)cc2n1C1CC(F)(F)C1]

6-Bromo-1-(3,3-difluorocyclobutyl)-2-methyl-1H-imidazo [4,5-b] pyridine (LXXXVIII): Grey solid (1.57 g, 6.178 mmol, 68.3% yield). ESIMS found C_{I i}thₒBrF₂N₃ m/z 302.1 (M+H).

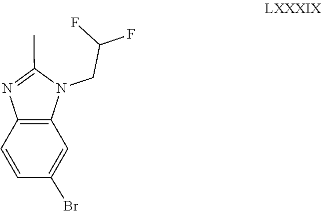

6-Bromo-1-(2,2-difluoroethyl)-2-methyl-1H-benzo[d]imidazole (LXXXIX): Beige solid (970 mg, 3.526 mmol, 79.0% yield). ESIMS found C₁₀H₉BrF₂N₂ m/z 275.0 (M+H).

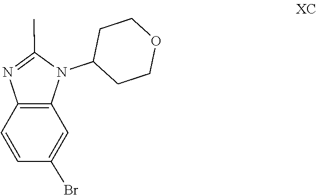 [SMILES: Cc1nc2ccc(Br)cc2n1C1CCOCC1]

6-Bromo-2-methyl-1-(tetrahydro -2H-pyran-4-yl)-1H-imidazo [4,5-b]pyridine (XC): Grey solid (722 mg, 2.438 mmol, 66.3% yield). ESIMS found C₁₂H₁₄BrN₃O m/z 296.0 (M+H).

Preparation of intermediate 6-bromo-1-ethyl-1H-benzo[d][1,2,3]triazole (XCIV) is depicted below in Scheme 19.

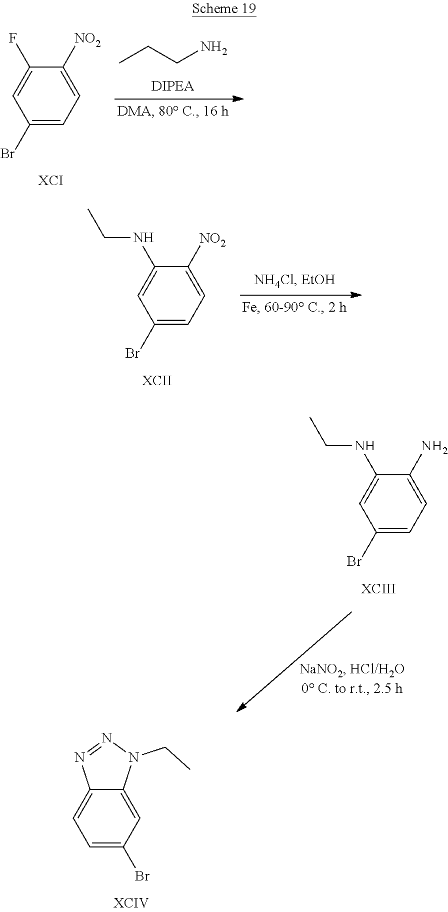

### Step 1

To a solution of 4-bromo-2-fluoro-1-nitrobenzene (XCI) (2 g, 9.1 mmol), propan-1-amine (0.742 g, 9.1 mmol) and DIPEA (2.35 g, 18.18 mmol) in DMA (50 mL). The mixture was stirred at 80° C. for 16 h. The mixture was diluted with EtOAc and then extracted with EtOAc (200 mL x 3) and H₂O. The organic layer was dried over anhydrous Na2SO₄, filtered, and concentrated. The crude residue was purified by silica gel column chromatography to give 5-bromo-N-ethyl-2-nitroaniline (XCII) (1.93 g, 7.87 mmol, 86.5% yield) as a yellow solid. ¹H NMR (400 MHz, DMSO-d₆) δppm 1.21 (t, J=7.2 Hz, 3H), 3.43-3.34 (m, 2H), 6.83 (dd, J=9.2, 2.0 Hz, 1H), 7.23 (d, J=2.0 Hz, 1H), 7.98 (d, J=9.2 Hz, 1H), 8.18-8.07 (m, 1H).

### Step 2

To a solution of 5-bromo-N-ethyl-2-nitroaniline (XCII) (1.8 g, 7.4 mmol) in EtOH (60 mL) was added a solution of NH₄C1 (2.09 g, 39.1 mmol) in H₂O (12 mL). The mixture was heated to 60° C., and Fe (2.07 g, 37 mmol) was added to the mixture. The mixture heated to 90° C. and reacted for 1 h. The mixture was diluted with EtOAc and then extracted with EtOAc (60 mL×3) and H₂O. The crude residue was purified by silica gel column chromatography to give 5-bromo-N¹-ethylbenzene-1,2-diamine (XCIII) (1.3 g, 6.04 mmol, 81.7% yield) as a yellow solid. ESIMS found for Cali iBrN₂m/z 215.1 (M+H).

### Step 3

To a solution of 5-bromo-N¹-ethylbenzene-1,2-diamine (XCIII) (1.2 g, 5.6 mmol) in concentrated HC1 (50 mL) was added NaNO₂ (425 mg, 6.1 mmol) in H₂O (10 mL). The mixture was allowed warm to room temperature and stirred for 1 h. After being cooled to 0° C., the reaction mixture was treated with 6 N NaOH until basic, the precipitate filtered, washed with H₂O, and dried to afford 6-bromo-1-ethyl-1H-benzo[d][1,2,3]triazole (XCIV) (1.1 g, 4.87 mmol, 86.9% yield) as a brown solid. ESIMS found for CsHsBrN₃ m/z 226.0 (M+H).

Preparation of intermediate 6-bromo-1-(2,2-difluoroethyl)-1H-benzo[d][1,2,3]triazole (XCVII) is depicted below in Scheme 20.

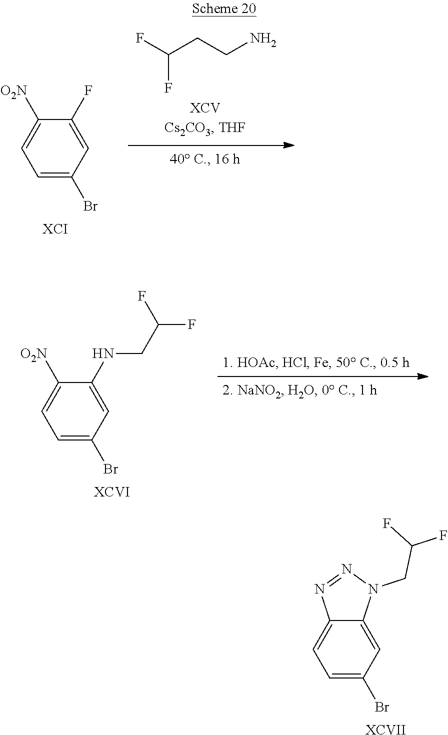

### Step 1

A solution of 4-bromo-2-fluoro-1-nitrobenzene (XCI) (20.0 g, 98.03 mmol) in THF (500.0 mL) was cooled to 0° C. Cs2CO₃ (63.9 g, 196.06 mmol) was added, 2,2-difluoroethan-1-amine (XCV) (36.6 g, 183.81 mmol) was added at 0° C. The reaction was warmed to 40° C. for 16 h. The reaction mixture was extracted with EtOAc (500 L x 3). The combined organics were washed with brine (500 mL x 3). The combined organic layers were dried with Na₂SO₄, filtered, and concentrated to give the crude product. The crude was purified by column chromatography on silica gel (10->20% EtOAc/petroleum ether) to give 5-bromo-N-(2,2-difluoroethyl)-2-nitroaniline (XCVI) (23 g, 81.83 mmol, 83.5% yield) as a yellow solid. ¹H NMR (400 MHz, DMSO-d₆) 6 3.99 (tdd, J=15.6, 6.6, 3.8 Hz, 2H), 6.29 (tt, J=55.4, 3.7 Hz, 1H), 6.96 (dd, J=9.2, 2.0 Hz, 1H), 7.51 (d, J=1.6 Hz, 1H), 8.05 (d, J=9.2 Hz, 1H), 8.33 (t, J=6.4 Hz, 1H); ESIMS found for C8H₇BrF2N2O₂ m/z 280.9 (M+H).

### Step 2

To a solution of 5-bromo-N-(2,2-difluoroethyl)-2-nitroaniline (XCVI) (12.0 g, 42.86 mmol) in HOAc/HC1 (500/50 mL) was added Fe (30.0 g, 428.62 mmol). The reaction mixture was stirred at 50° C. for 30 minutes, then cooled to room temperature and filtered. NaNO₂ (3.0 g, 53.58 mmol) in water (20 mL) was then added dropwise into above acid solution at 0° C. The reaction solution was stirred for 1 h at 0° C. The reaction mixture was concentrated to dryness, the reaction mixture was poured into EtOAc (300 mL) and H₂O (300 mL). The pH was adjusted >7 with NaHCO₃. The reaction mixture was extracted with EtOAc (500 mL x 3). The combined organics were washed with brine (500 mL x 3). The organic layers was concentrated, dried over Na2SO₄, filtered, and concentrated to give the crude. The crude was purified by column chromatography on silica gel (10->50% EtOAc/petroleum ether) to give 6-bromo-1-(2,2-difluoroethyl)-1H-benzo[d][1,2,3]triazole (XCVII) (5 g, 19.08 mmol, 44.5%) as a brown solid. ¹H NMR (400 MHz, DMSO-d₆) 6 5.41-5.29 (m, 2H), 6.61 (tt, J=54.2, 3.2 Hz, 1H), 7.60 (dd, J =8.8, 1.7 Hz, 1H), 8.08 (d, J=8.8 Hz, 1H), 8.31 (d, J=1.0 Hz, 1H); ESIMS found for C₈H₆BrF₂N₃ m/z 261.9 (M+H).

Preparation of intermediate 6-bromo-7-fluoro-1-isopropyl-2-methyl-1H-benzo[d]imidazole (CI) is depicted below in Scheme 21.

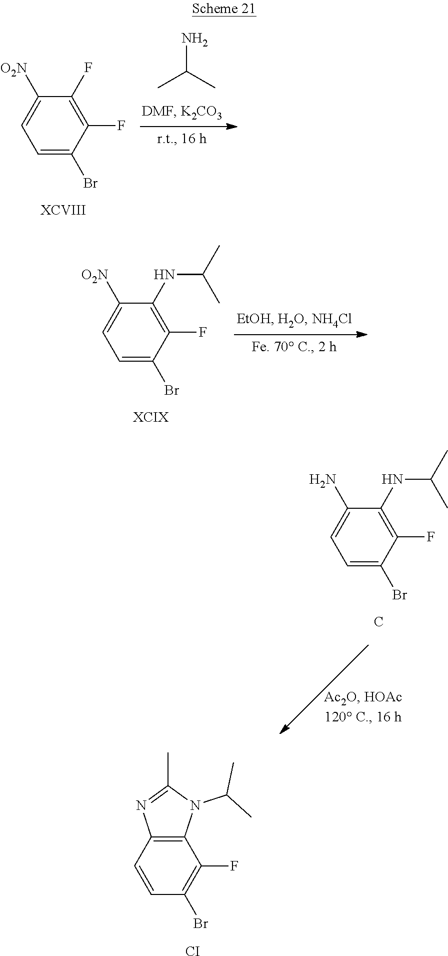

### Step 1

A mixture of 2-aminopropane (0.4 mL, 4.66 mmol), 1-bromo-2,3-difluoro-4-nitrobenzene (XCVIII) (1 g, 4.2 mmol) and K₂CO₃ (1.16 g, 8.41 mmol) in DMF (15 mL) was stirred at room temperature for 16 h. The reaction mixture was added to water (200 mL), stirred for 1 h and the resulting solids were collected by filtration and dried under high vacuo to obtain 3-bromo-2-fluoro-6-nitro-N-propan-2-ylaniline (XCIX) (670.0 mg, 2.42 mmol, 57.5% yield) as a yellow solid which was used for next step without purification.

### Step 2

A mixture of 3-bromo-2-fluoro-6-nitro-N-propan-2-ylaniline (XCIX) (0.67 g, 2.42 mmol) Fe (1.58 g, 24.19 mmol) and NH₄C1 (1.94 g, 36.26 mmol) was taken in a mixture of EtOH (8 mL), and water (3 mL) and the mixture was heated to 70° C. for 4 h. The reaction mixture was cooled, filtered through celite, filtrates were taken into EtOAc, washed with water then brine, dried over anhydrous Na₂SO₄, filtered, and concentrated under reduced pressure. The residue was purified on column chromatography (0→40% EtOAc/hexanes) on a column filled with 24 g of silica gel to obtain 4-bromo-3-fluoro-2-N-propan-2-ylbenzene-1,2-diamine (C) (495.0 mg, 2.003 mmol, 82.8% yield) as a light purple oil. ESIMS found for C₉E1₁₂BrFN₂m/z 247.0 (M+H).

### Step 3

A solution of 4-bromo-3-fluoro-2-N-propan-2-ylbenzene-1,2-diamine (C) (0.5 g, 2 mmol) and Ac2O (0.23 mL, 2.43 mmol) in HOAc (6 mL) was heated to 120° C. for 16 h. The reaction mixture was concentrated, the residue partitioned between EtOAc/1 N NaOH, organics separated, washed with water and brine. The organics were dried over anhydrous Na2SO₄, solvents concentrated and dried under high vacuo. The residue was purified via column chromatography (0→80% EtOAc/hexanes) on a column filled with 24 g of silica gel to give 6-bromo-7-fluoro-2-methyl-1-propan-2-ylbenzimidazole (CI) (204 mg, 0.752 mmol, 37.6% yield) as a light-yellow solid. ESIMS found for C₁₁H₁₂BrFN₂ m/z 271.0 (M+H).

The following intermediates were prepared in accordance with the procedure described in the above Scheme 21.

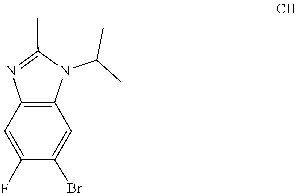

6-Bromo-5-fluoro-14 sopropyl-2-methyl-1H-benzo [d]imidazole (CII): Light-yellow solid (985.0 mg, 3.633 mmol, 57.2% yield). ^{l}ESIMS found for C₁₁H₁₂BrFN₂ m/z 271.0 (M+H).

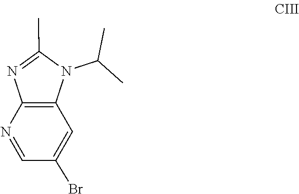

6-Bromo-1-isopropyl-2-methyl-1H-imidazo[4,5-b]pyridine (CIII): Dark brown solid (1.57 g, 6.178 mmol, 68.3% yield). ^{l}ESIMS found for C₁₀H₁₂BrN₃ m/z 254.0 (M+H).

Preparation of intermediate 5-chloro-3-(2,2-difluoroethyl)-2-methyl-3H-imidazo[4,5-b]pyridine (CV) is depicted below in Scheme 22.

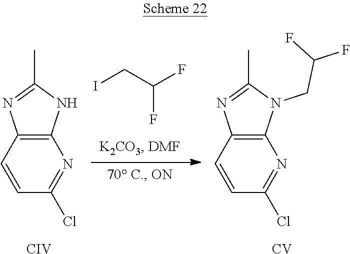

### Step 1

A mixture of 5-chloro-2-methyl-3H-imidazo[4,5-b]pyridine (CIV) (commercially available from Ambeed, Inc.) (1 g, 5.97 mmol), 1,1-difluoro-2-iodoethane (1.38 g, 7.19 mmol) and K2CO₃ (1.65 g, 11.94 mmol) in DMF (20 mL) was heated to 70° C. overnight. LCMS showed the formation of two isomers. The desired isomer was the major product. The reaction mixture was cooled, solvents concentrate, and the residue partitioned between EtOAc/water. The organics were separated, washed with brine, dried over anhydrous Na2SO₄, and the solvents were concentrated. The crude was purified by ISCO (0-400% EtOAc/hexanes) to obtain 5-chloro-3-(2,2-difluoroethyl)-2-methylimidazo [4,5-b]pyridine (CV) (830 mg, 3.583 mmol, 60.1% yield) as a beige solid. ESIMS found for C₉H₈ClF₂N₃ m/z 232.0 (M+H).

The following intermediates were prepared in accordance with the procedure described in the above Scheme 22.

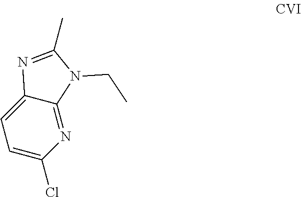

5-Chloro-3-ethyl-2-methyl-3H-imidazo[4,5-b]pyridine (CVI): Dark brown solid (466 mg, 2.382 mmol, 79.8% yield). ESIMS found for C₉H₁₀ClN₃ m/z 196.05 (M+H).

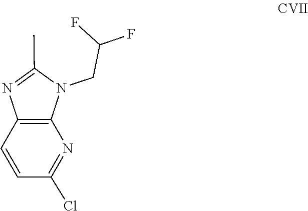

5-Chloro-3-(2,2-difluoroethyl)-2-methyl-3H-imidazo[4,5-b]pyridine (CVII):

Beige solid (830 mg, 3.583 mmol, 60.1% yield). ESIMS found C₉H₈ClF₂N₃m/z 232.0 (M+H).

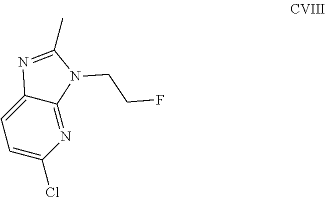

5-Chloro-3-(2-fluoroethyl)-2-methyl-3H-imidazo[4,5-b]pyridine (CVIII): Beige solid (220 mg, 1.030 mmol, 57.5% yield). ESIMS found C₉H₉ClFN₃m/z 214.05 (M+H).

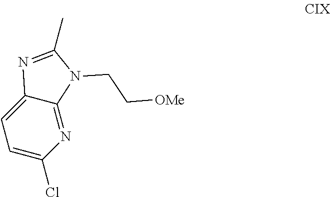

5-Chloro-3-(2-methoxyethyl)-2-methyl-3H-imidazop,5-blpyridine (CIX):

Beige solid (195 mg,0.864 mmol, 48.3% yield). ESIMS found C₁₀th₂ClN₃O m/z 226.1 (M+H).

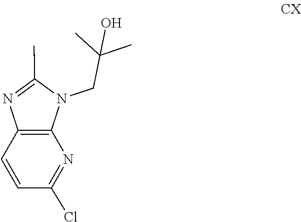 [SMILES: Cc1nc2ccc(Cl)nc2n1CC(C)(C)O]

1-(5-Chloro-2-methyl-3H-imidazo [4,5-b]pyridin-3-yl)-2-methylpropan-2-ol (CX): White solid (229.9 mg, 0.959 mmol, 39.7% yield). ¹II NMR (499 MHz, DMSO-d₆) δppm 1.13 (6 H, s), 2.63 (3 H, s), 4.11 (2 H, s), 4.80 (1 H, s), 7.25 (1 H, d, J=8.21 Hz), 7.96 (1 H, d, J=8.21 Hz); ESIMS found CI ith4ClN30 m/z 240.1 (M+H).

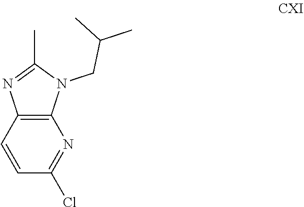

5-Chloro-3-isobutyl-2-methyl-3H-imidazop,5-blpyridine (CXI): White solid (206.8 mg, 0.925 mmol, 38.7% yield). ¹H NMR (499 MHz, DMSO-d₆) δppm 0.87 (6 H, d, J=6.57 Hz), 2.22 (1 H, dquin, J=13.89, 7.07, 7.07, 7.07, 7.07 Hz), 2.58 (3 H, s), 4.01 (2 H, d, J=7.67 Hz), 7.26 (1 H, d, J=8.21 Hz), 7.98 (1 H, d, J=8.21 Hz); ESIMS found C₁₁H₁₄ClN₃m/z 224.1 (M+H).

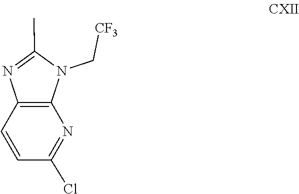 [SMILES: Cc1nc2ccc(Cl)nc2n1CC(F)(F)F]

5-Chloro-2-methyl-3-(2,2,2-trifluoroethyl)-3H-imidazo [4,5-b]pyridine (CXII): Beige solid (372 mg, 1.490 mmol, 50.0% yield). ESIMS found C₉H₇ClF₃N₃ m/z 250.0 (M+H).

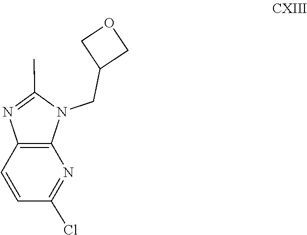 [SMILES: Cc1nc2ccc(Cl)nc2n1CC1COC1]

5-Chloro-2-methyl-3-(oxetan-3-ylmethyl)-3H-imidazo [4,5-b]pyridine (CXIII): Light brown solid (289.7 mg, 1.219 mmol, 40.4% yield). ¹H NMR (499 MHz, DMSO-d₆) δppm 2.59 (3 H, s), 3.43-3.56 (1 H, m), 4.47 (2 H, t, J=6.02 Hz), 4.52 (2 H, d, J=7.67 Hz), 4.62 (2 H, dd, J=7.67, 6.02 Hz), 7.27 (1 H, d, J=8.21 Hz), 7.98 (1 H, d, J=8.21 Hz); ESIMS found C₁₁H₁₂ClN₃O m/z 238.1 (M+H).

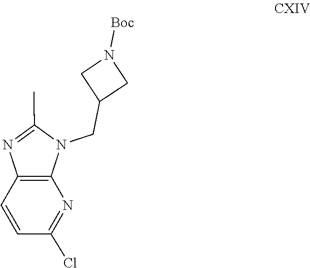 [SMILES: Cc1nc2ccc(Cl)nc2n1CC1CN(C(=O)OC(C)(C)C)C1]

tent-Butyl 3-45-chloro-2-methyl-3H-imidazo [4,5-1)] pyridin-3-yl)methyl)azetidine-1-carboxylate (CXIV): Off-white amorphous solid (532.6 mg, 1.581 mmol, 52.1% yield). ESIMS found C₁₆H₂₁ClN₄O₂m/z 337.1 (M+H).

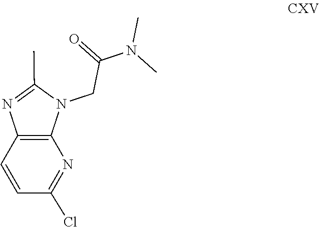 [SMILES: Cc1nc2ccc(Cl)nc2n1CC(=O)N(C)C]

245-Chloro-2-methyl-3H-imidazo [4,5-b] pyridin-3-yl)-N,N-dimethylacetamide (CXV): Off-white solid (474.4 mg, 1.877 mmol, 62.6% yield). ESIMS found C₁₁H₁₃ClN₄Om/z 253.1 (M+H).

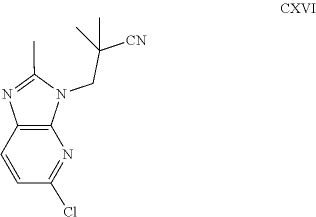 [SMILES: Cc1nc2ccc(Cl)nc2n1CC(C)(C)C#N]

3-(5-Chloro-2-methyl-3H-imidazo[4,5-b]pyridin-3-yl)-2,2-dimethylpropanenitrile (CXVI): Off-white amorphous solid (59.1 mg, 0.238 mmol, 7.9% yield). ESIMS found C₁₂H₁₃ClN₄ m/z 249.1 (M+H).

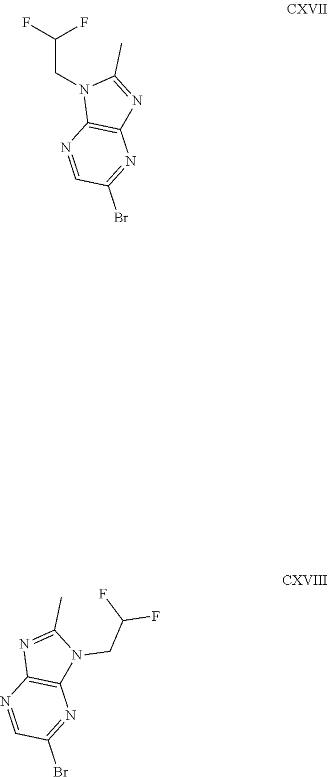

An inseparable mixture of 5-bromo-1-(2,2-difluoroethyl)-2-methylimidazo[4,5-b]pyrazine (CXVII) and 5-bromo-3-(2,2-difluoroethyl)-2-methylimidazo[4,5-14]yrazine (CXVIII) (556 mg, 2.007 mmol, 40.7% yield) as a brown solid. ESIMS found C₈H₇BrF₂N₄ m/z 277.0 (M+H). Final compounds were separated by chiral supercritical fluid chromatography (SFC) (System: Waters SFC 150; Column size: 250*25 mm 10 μm; Mobile Phase A: Supercritical CO₂; Mobile Phase B: MeOH (+0.1% 7.0mol/l Ammonia in MeOH); A:B ratio: 50:50; Flow: 70 mL/min; Column temp: r.t.)

Preparation of intermediate (5-bromopyrazolo [1,5-a]pyridin-3-yl)(4-methylpiperazin-1-yl)methanone (CXXI) is depicted below in Scheme 23.

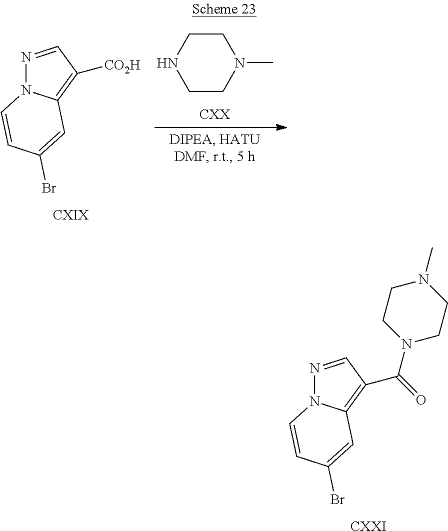

### Step 1

A mixture of 5-bromopyrazolo[1,5-a]pyridine-3-carboxylic acid (CXIX) (commercially available from Advanced ChemBlocks Inc.) (300 mg, 1.24 mmol), DIPEA (0.44 mL, 2.53 mmol), and HATU (0.47 g, 1.24 mmol) in DMF (4 mL) was stirred at room temperature for 5 min. Then, 1-methylpiperazine (CXX) (0.28 mL, 2.52 mmol) was added and the reaction mixture was continued at room temperature for 5 h. The solvents were concentrated in vacuo, the residue taken into EtOAc, washed with water, saturated aqueous NaHCO₃, water, and brine. The organic layers were dried over anhydrous Na2SO₄, then concentrated and under high vacuo to obtain crude (5-bromopyrazolo [1,5-a] pyridin-3-yl)(4-me thylpiperazin-1-yl)methanone (CXXI) (335 mg, 1.037 mmol, 83.3% yield) as a brown gummy solid, which was used in the next step without purification. ESIMS found for C₁₃H₁₅BrN₄O m/z 323.0 (⁷⁹BrM+H).

The following intermediates were prepared in accordance with the procedure described in the above Scheme 23.

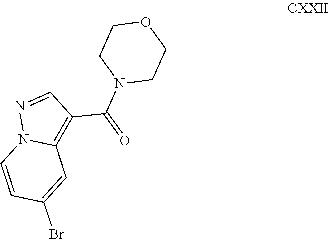 [SMILES: O=C(c1cnn2ccc(Br)cc12)N1CCOCC1]

(5-Bromopyrazolo [1,5-a] pyridin-3-yl)(morpholino)methanone (CXXII): Off-white solid (123.0 mg, 0.397 mmol, 95.6% yield). ¹ESIMS found for C₁₂H₁₂BrN₃O₂ m/z 310.95 (M+H).

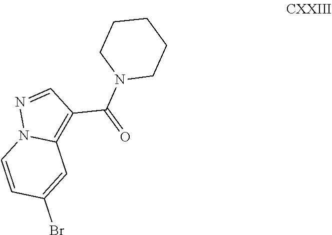 [SMILES: O=C(c1cnn2ccc(Br)cc12)N1CCCCC1]

(5-Bromopyrazolo [1,5-a] pyridin-3-yl)(piperidin-1-yl)methanone (CXXIII): Light brown solid (121.0 mg, 0.393 mmol, 94.6% yield). ¹ESIMS found for C₁₃H₁₄BrN₃O m/z 308.0 (M+H).

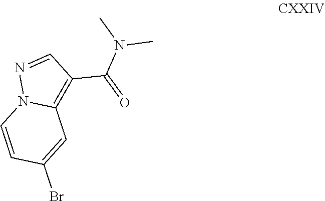

5-Bromo-N,N-dimethylpyrazolo [1,5-a]pyridine-3-carboxamide (CXXIV): White sold (126 mg, 0.470 mmol, 56.6% yield). ¹ESIMS found for C₁₀tl₁₀BrN₃O m/z 268.0 (M+H).

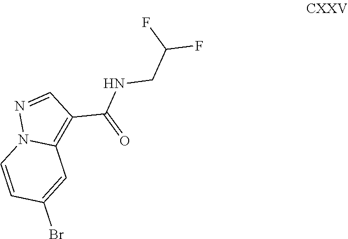 [SMILES: O=C(NCC(F)F)c1cnn2ccc(Br)cc12]

5-Bromo-N-(2,2-difluoroethyl)pyrazolo [1,5-a] pyridine-3-carboxamide (CXXV): White sold (506 mg, 1.66 mmol, 87.2% yield). ¹ESIMS found for C₁₀H₈BrF₂N₃O m/z 304.0 (M+H).

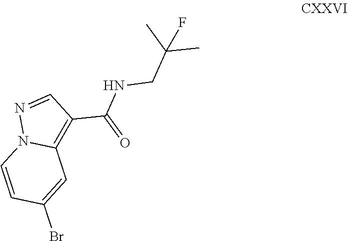 [SMILES: CC(C)(F)CNC(=O)c1cnn2ccc(Br)cc12]

5-Bromo-N-(2-fluoro-2-methylpropyl)pyrazolo [1,5-a] pyridine-3-carboxamide (CXXVI): White sold (250 mg, 0.796 mmol, 95.9% yield). ¹ESIMS found for C₁₂H₁₃BrFN₃O m/z 314.0 (M+H).

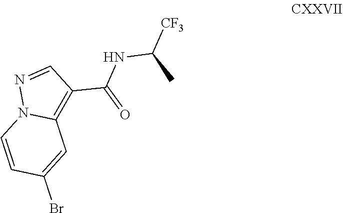 [SMILES: C[C@@H](NC(=O)c1cnn2ccc(Br)cc12)C(F)(F)F]

(R)-5-Bromo-N-(1,1,1-trifluoropropan-2-yl)pyrazolo [1,5-a] pyridine-3-carboxamide (CXXVII): White sold (280 mg, 0.833 mmol, 100.4% yield). ¹ESIMS found for C₁₁H₉BrF₃N₃O m/z 336.0 (M+H).

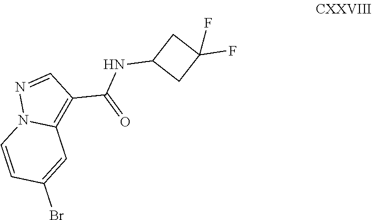 [SMILES: O=C(NC1CC(F)(F)C1)c1cnn2ccc(Br)cc12]

5-Bromo-N-(3,3-difluorocyclobutyppyrazolo [1,5-a] pyridine-3-carboxamide (CXXVIII): White sold (245 mg, 0.742 mmol, 89.4% yield). ¹ESIMS found for C₁₂HioBrF₂N₃O m/z 330.0 (M+H).

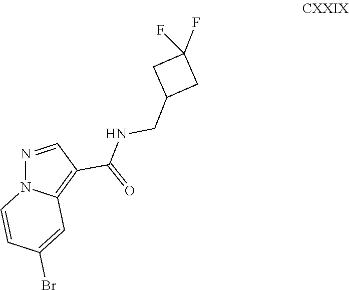 [SMILES: O=C(NCC1CC(F)(F)C1)c1cnn2ccc(Br)cc12]

5-Bromo-N-((3 ,3-difluorocyclobutyl)methyl)pyrazolo [1,5-a] pyridine-3-carboxamide (CXXIX): White sold (253 mg, 0.735 mmol, 88.6% yield). ¹ESIMS found for C₁₃H₁₂BrF2N30 m/z 344.0 (M+H).

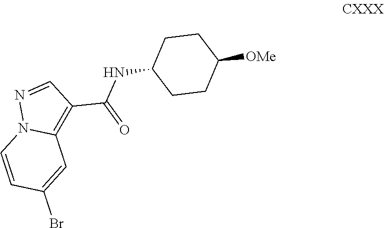 [SMILES: CO[C@H]1CC[C@H](NC(=O)c2cnn3ccc(Br)cc23)CC1]

trans-5-Bromo-N-(4-methoxycyclohexyl)pyrazolo [1,5-a] pyridine-3-carboxamide (CXXX): White sold (180 mg, 0.511 mmol, 94.8% yield). ¹ESIMS found for C₁₅H₁₈BrN₃O₂ m/z 352.1 (M+H).

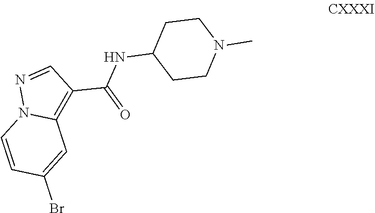 [SMILES: CN1CCC(NC(=O)c2cnn3ccc(Br)cc23)CC1]

5-Bromo-N-(1-methylpiperidin-4-yl)pyrazolo [1,5-a] pyridine-3-carboxamide (CXXXI): White sold (157.0 mg, 0.466 mmol, 112.2% yield). ¹ESIMS found for C₁₄H₁₇BrN₄O m/z 337.0 (M+H).

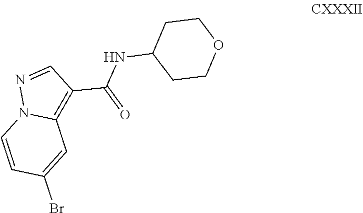 [SMILES: O=C(NC1CCOCC1)c1cnn2ccc(Br)cc12]

5-Bromo-N-(tetrahydro-2H-pyran-4-yl)pyrazolo [1,5-a] pyridine-3-carboxamide (CXXXII): White solid (85 mg, 0.262 mmol, 63.2% yield). ¹ESIMS found for C₁₃H₁₄BrN₃O₂ m/z 324.0 (M+H).

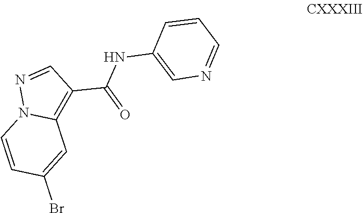 [SMILES: O=C(Nc1cccnc1)c1cnn2ccc(Br)cc12]

5-Bromo-N-(pyridin-3-yl)pyrazolo [1,5-a] pyridine-3-carboxamide (CXXXIII): Light brown solid (86.0 mg, 0.271 mmol, 65.4% yield). ¹ESIMS found for C₁₃H₉BrN₄Om/z 316.9 (M+H).

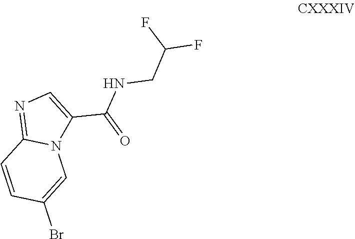 [SMILES: O=C(NCC(F)F)c1cnc2ccc(Br)cn12]

6-Bromo-N-(2,2-difluoroethyl)imidazo[1,2-a]pyridine-3-carboxamide (CXXXIV): Light yellow solid (240 mg, 0.789 mmol, 95.1% yield). ¹ESIMS found for C₁₀H₈BrF2N30 m/z 304.0 (M+H).

Preparation of intermediate 5-(tributylstannyl)pyrazolo[1,5-a]pyrimidine (CXXXVI) is depicted below in Scheme 24.

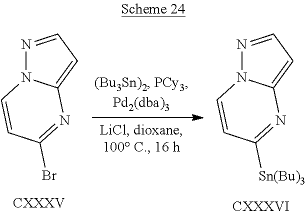

### Step 1

To a solution of 5-bromopyrazolo[1,5-a]pyrimidine (CXXXV) (commercially available from Combi-Blocks Inc.) (3.0 g, 15.15 mmol) in 1,4-dioxane (300 mL) under N2 was added PCy₃ (430 mg, 1.53 mmol), Pd₂(dba)₃ (700 mg, 0.76 mmol), LiCl (3.85 g, 90.92 mmol) and (Bu₃Sn)₂ (9.6 mL, 19. mmol) and the mixture was heated to 100° C. for 16 h. The reaction mixture was cooled to room temperature, filtered and the filtrates were added to water (600 mL) and extracted with EtOAc. The organics were separated, washed with brine, dried over anhydrous MgSO₄, solvents concentrated, and the residue was purified by ISCO (liquid loading) (0→50% EtOAc/hexanes) to obtain 5-(tributylstannyl)pyrazolo [1,5-a]pyrimidine (CXXXVI) (1.27 g, 3.111 mmol, 20.5% yield) as a yellow liquid. ESIMS found for C₁₈₃₁N₃Sn m/z 410.2 (M+H).

Preparation of intermediate 6-bromo-4-(4-methylpiperazin-1-yl)quinazoline (CXXXVIII) is depicted below in Scheme 25.

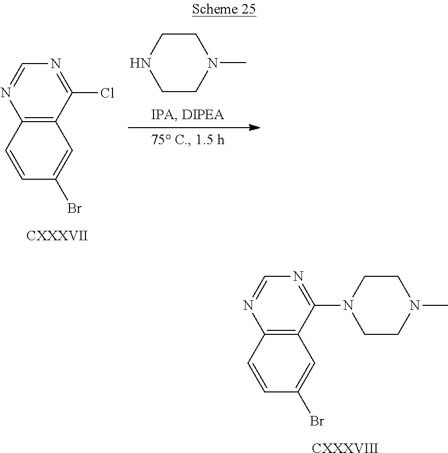

### Step 1

To a stirred suspension of 6-bromo-4-chloroquinazoline (CXXXVII) (commercially available from Enamine Ltd) (0.5 g, 2.05 mmol) in IPA (5 mL) was added DIPEA (0.72 mL, 4.13 mmol) and 1-methylpiperazine (632.1 mg, 6.310 mmol). The mixture becomes clear solution in few minutes and was heated to 75° C. for 1.5 h. The solvents were concentrated, the residue partitioned between EtOAc/water, organics separated, washed with brine, dried over anhydrous Na2SO₄, solvents concentrated and dried under high vacuo to obtain crude 6-bromo-4-(4-methylpiperazin-1-yl)quinazoline (CXXXVIII) (645.0 mg, 2.100 mmol, 102.3% yield) as a thick brown gum which was used for next the step without purification. ESIMS found for C₁₃H₁₅BrN₄m/z 307.0 (M+H).

Preparation of intermediate 7-bromo-2-((1-methylpiperidin-4-yl)oxy)quinoxaline (CXLI) is depicted below in Scheme 26.

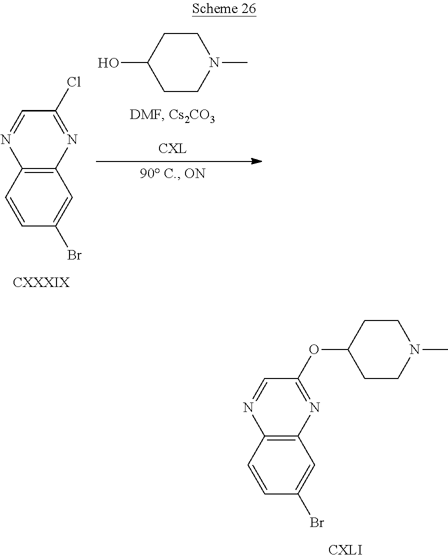

### Step 1

A mixture of 1-methyl-4-piperidinol (CXL) (0.36 g, 3.13 mmol), Cs₂CO₃ (1.34 g, 4.11 mmol) and 7-bromo-2-chloro-quinoxaline (CXXXIX) (commercially available from Enamine Ltd) (0.5 g, 2.05 mmol) in DMF (6 mL) was heated to 90° C. overnight. The solvents were concentrated, and the residue partitioned between EtOAc/water. The organic layer was separated, washed with brine, dried over anhydrous Na2SO₄, concentrated. The residue was purified by ISCO (100% CHCl₃→50% CHCl₃/10%7N NH₃ MeOH/CHCl₃). The pure fractions were concentrated, dried under high vacuo to obtain 7-bromo-2-(1-methylpiperidin-4-yl)oxyquinoxaline (CXLI) (293.0 mg, 0.909 mmol, 44.3% yield) as a beige solid. ESIMS found for C₁₄H₁₆BrN3O m/z 322.0 (M+H).

Preparation of intermediate cis-4-(methoxy-d₃)cyclohexan-1-amine (CXLVII) is depicted below in Scheme 27.

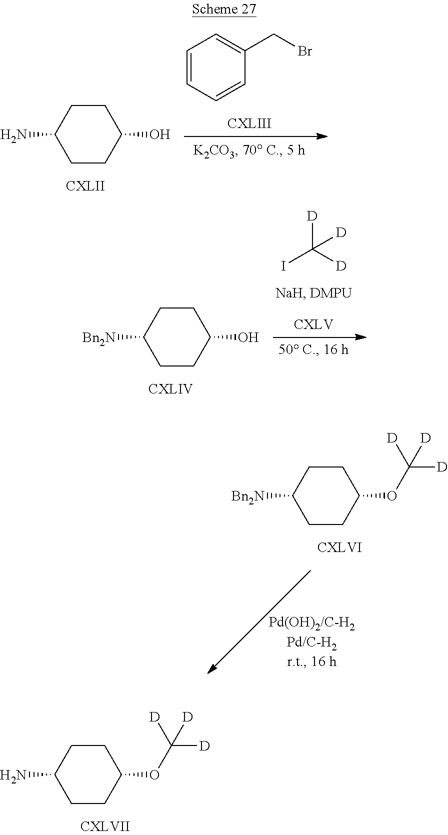

### Step 1

To a solution of cis-4-aminocyclohexan-1-ol (CXLII) (5 g, 32.9 mmol), (bromomethyl)benzene (CXLIII) (11.25 g, 65.8 mmol) in MeCN (80 mL) was added K2CO₃(13.64 g, 98.7 mmol). The mixture was stirred at 70° C. for 5 h. The reaction mixture was concentrated under reduced pressure to remove MeCN. The mixture was diluted with EtOAc and then extracted with EtOAc (100 mL x 3) and H₂O. The combined organic layers were concentrated, and the crude residue was purified by silica gel column chromatography (0->30% EtOAc/PE) to give the cis-4-(dibenzylamino)cyclohexan-1-ol (CXLIV) (8.0 g, 27.08 mmol, 82.3% yield) as a white solid. ESIMS found for C₂₀1⁻1₂₅NO m/z 296.4 (M+H).

### Step 2

To a solution of cis-4-(dibenzylamino)cyclohexan-1-ol (CXLIV) (8.0 g, 27.08 mmol) in DMPU (80 mL) was added slowly NaH (5.98 g, 149.7 mmol) under nitrogen atmosphere with continuous stirring. The reaction mixture was stirred at room temperature for 1 h. Then iodomethane-d₃ (CXLV) (10.85 g, 74.86 mmol) was added at room temperature over a period of 10 min. After complete addition, the reaction mixture was stirred for 16 h at 50° C. The reaction mixture was then quenched with saturated aqueous NH₄C1 (300 mL) and stirred for 10 min. The mixture was diluted with EtOAc and then extracted with EtOAc (300 mL×3) and H₂O. The crude residue was purified by silica gel column chromatography (0->20% EtOAc/PE) to yield cis-N,N-dibenzyl-4-(methoxy-d₃)cyclohexan-1-amine (CXLVI) (6 g, 19.202 mmol, 70.9% yield) as a colorless oil. ESIMS found for C_{2 1}14₂₄D₃NO m/z 313.0 (M+H).

### Step 3

To a solution of cis-N,N-dibenzyl-4-(methoxy-d₃)cyclohexan-1-amine (CXLVI) (200 mg, 0.64 mmol) in EtOH (5 mL) was added Pd(OH)₂/C (50 mg) and Pd/C (50 mg). The mixture was stirred at room temperature for 16 h. The mixture was filtered through ^{Celite}® and washed with EtOH. The reaction mixture was concentrated under reduced pressure to give the cis-4-(methoxy-d₃)cyclohexan-1-amine (CXLVII) (76.4 mg, 0.578 mmol, 90.3% yield) as a colorless oil. ¹H NMR (400 MHz, DMSO-d₆) δppm 1.51-1.40 (m, 4H), 1.67-1.56 (m, 4H), 1.86 (td, J=9.8, 4.6 Hz, 2H), 2.71 (tt, J=10.8, 5.4 Hz, 1H), 3.34 (td, J=4.8, 2.4 Hz, 1H); ESIMS found for C71⁻112133NO m/z 133.0 (M+H).

The following intermediates were prepared in accordance with the procedure described in the above Scheme 27.

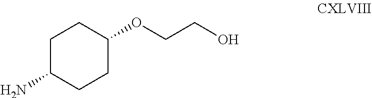 [SMILES: N[C@H]1CC[C@@H](OCCO)CC1]

2-((cis-4-Aminocyclohexyl)oxy)ethan-1-ol (CXLVIII): Colorless oil (0.5 g, 3.14 mmol, 67.4% yield). ESIMS found for C₈1-1₁₇NO₂ m/z 160. (M+H).

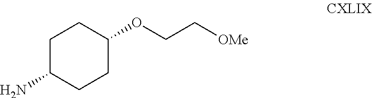 [SMILES: COCCO[C@H]1CC[C@@H](N)CC1]

cis-4-(2-Methoxyethoxy)cyclohexan-1-amine (CXLIX: Colorless oil (1.5 g, 8.65 mmol, 76.6% yield). ESIMS found for C₉H₁₉NO₂ m/z 174.1 (M+H).

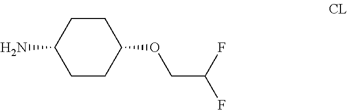 [SMILES: N[C@H]1CC[C@@H](OCC(F)F)CC1]

cis-4-(2,2-Difluoroethoxy)cyclohexan-1-amine (CL): White solid (1.352 g, 7.54 mmol, 90.3% yield). ESIMS found for C₈1-1₁₅F₂N0 m/z 180.1 (M+H).

Preparation of intermediate cis-4-(difluoromethoxy)cyclohexan-1-amine (CLIII) is depicted below in Scheme 28.

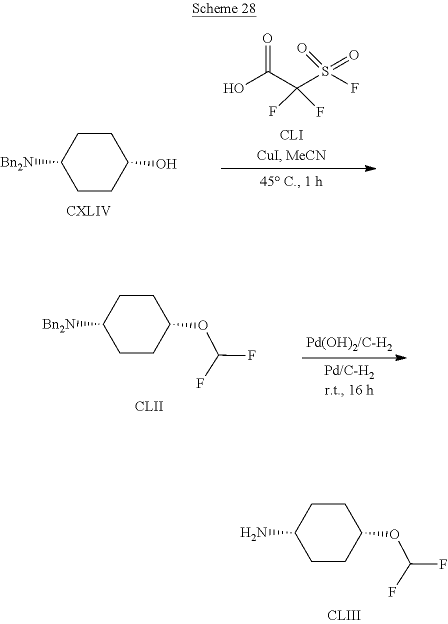

### Step 1

To a solution of cis-4-(dibenzylamino)cyclohexan-1-ol (CXLIV) (50 mg, 0.170 mmol), CuI (6.5 mg, 0.034 mmol) in MeCN (5 mL) and heated to 45° C. under nitrogen atmosphere for 5 min. To this mixture was added a solution of 2,2-difluoro-2-(fluorosulfonyl)acetic acid (CLI) (60 mg, 0.339 mmol) in (2 mL) MeCN over 10 min. Then the mixture was stirred at 45° C. for 1 h. Volatile components were then removed via evaporation and the residue was diluted with EtOAc (100 mL) and a 1:1 mixture of water/saturated aqueous NaHCO₃ (100 mL). The resulting biphasic mixture containing solids was filtered through a sintered glass Buchner funnel. The filtrate layers were separated, and the aqueous layer was extracted with EtOAc (50 mL). The combined EtOAc layers were washed with a 1:1 mixture of brine/water (50 mL), dried over anhydrous MgSO₄, filtered, and concentrated to an oil. The crude oil was purified by silica gel chromatography (0→30% EtOAc/hexanes). Product containing fractions were combined and concentrated to afford the cis-N,N-dibenzyl-4-(difluoromethoxy)cyclohexan-1-amine (CLII) (25 mg, 0.072 mmol, 42.3% yield) as an oil that solidified to an off-white solid. ESIMS found for C₂₁t1₂₅F₂NO m/z 346.1 (M+H).

### Step 2

To a solution of cis-N,N-dibenzyl-4-(difluoromethoxy)cyclohexan-1-amine (CLII) (2.8 g, 8.11 mmol) in THF (60 mL) was added Pd (OH)₂/C (1.4 g) and Pd/C (1.4 g). The mixture was stirred at room temperature for 16 h. The mixture was filtered through Celite® and washed with THF. The reaction mixture was concentrated under reduced pressure to afford cis-4-(difluoromethoxy)cyclohexan-1-amine (CLIII) (1.05 g, 6.36 mmol, 78.4% yield) as a colorless oil. ESIMS found for C4⁻113F2NO m/z 166.1 (M+H).

### Example 1

Preparation of 2-cyclobutyl-5-(imidazo[1,2-a]pyridin-6-yl)-7H-pyrrolo[2,3-d]pyrimidine (103) is depicted below in Scheme 29.

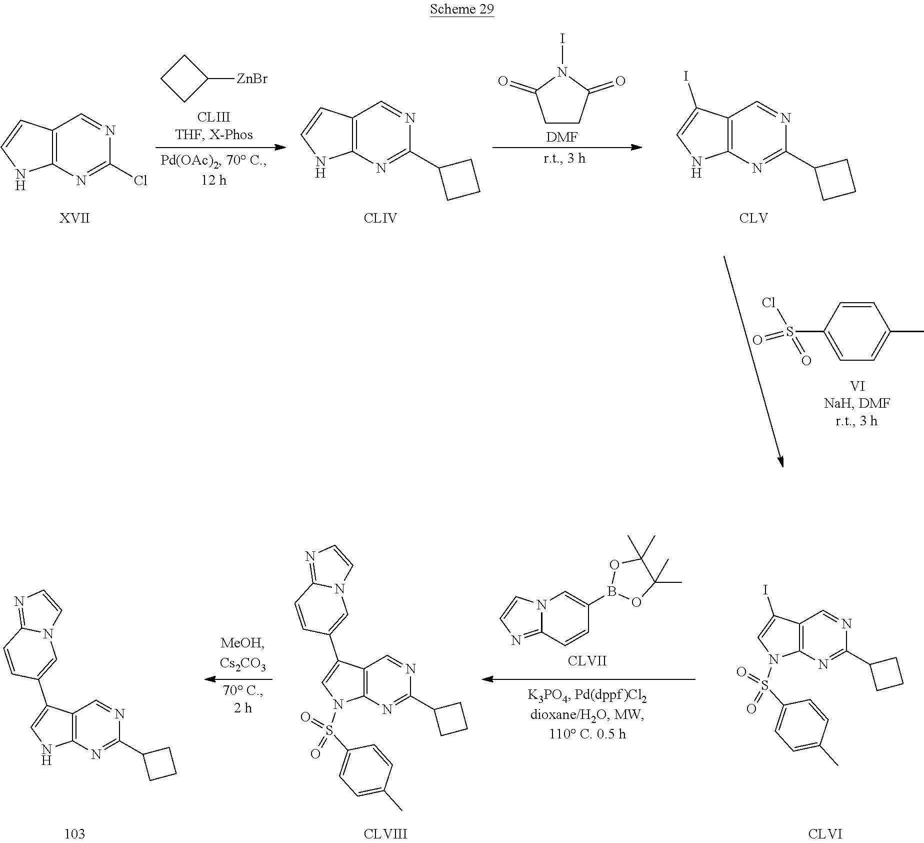

### Step 1

To a mixture of 2-chloro-7H-pyrrolo[2,3-d]pyrimidine (XVII) (1.g, 6.51 mmol), Pd(OAc)₂ (90 mg, 0.400 mmol) and X-Phos (290 mg, 0.660 mmol) in dry THF (5 mL) was added 0.5 M solution of bromo(cyclobutyl)zinc (CLIII) (20 mL, 10 mmol). N₂ gas was purged for 5 min and the mixture was heated to 70° C. for 12 h. The reaction mixture was cooled to room temperature, absorbed on silica gel and was purified by ISCO (0→6% 7N NH₃ in MeOH/CHCl₃) to obtain 2-cyclobutyl-7H-pyrrolo [2,3-d]pyrimidine (CLIV) (1.1 g, 6.351 mmol, 97.5% yield) as a beige solid. ESIMS found for Cₗₒth m/z 174.1 (M+H).

### Step 2

A mixture of 2-cyclobutyl-7H-pyrrolo [2,3-d]pyrimidine (CLIV) (1.1 g, 6.35 mmol), 1-iodopyrrolidine-2,5-dione (1.57 g, 6.98 mmol) in DMF (10 mL) was stirred at room temperature for 3 h. The reaction mixture was added to water (50 mL) and stirred for 30 min. The resulting solid were collected by filtration and dried under high vacuo to obtain 2-cyclobutyl-5-iodo-7H-pyrrolo[2,3-d]pyrimidine (CLV) (1.8 g, 6.018 mmol, 94.8% yield) as a beige solid. ESIMS found for CiothoIN3m/z 300.0 (M+H).

### Step 3

Sodium hydride (150 mg, 3.75 mmol) was added portion wise to a cold (0° C.) mixture of 2-cyclobutyl-5-iodo-7H-pyrrolo[2,3-d]pyrimidine (CLV) (440 mg, 1.47 mmol) in dry DMF (10 mL) under nitrogen atmosphere. The mixture was stirred for 30 min at 0° C. Tosyl chloride (VI) (400 mg, 2.1 mmol) was added portion wise and stirring was continued for 1 h at 0° C. Reaction mixture was slowly diluted with water, precipitated solids were filtered, washed with aqueous saturated NaHCO₃ and water. Collected solids were dried under high vacuum to obtain 2-cyclobutyl-5-iodo-7-(4-methylphenyl) sulfonylpyrrolo [2,3-d] pyrimidine (CLVI) (556 mg, 1.227 mmol, 83.4% yield) as a beige solid. ESIMS found for C₁₇H₁₆IN₃O₂Sm/z 454.05 (M+H).

### Step 4

A mixture of 6-(4,4,5,5-tetramethyl-1,3,2-dioxaborolan-2-yl)imidazo [1,2-a] pyridine (CLVII) (25 mg, 0.100 mmol), 2-cyclobutyl-5-iodo-7-(4-methylphenyl)sulfonylpyrrolo [2,3-d]pyrimidine (CLVI) (38 mg, 0.080 mmol), Pd(dppf)Cl₂ (3.6 mg, 0.0044 mmol), and 2 M aqueous solution of K₃PO₄ (0.12 mL, 0.240 mmol) in 1,4-dioxane (5 mL) was purged with N₂ gas for 5 min. The mixture was heated with microwave irradiation at 110° C. for 30 min. The reaction mixture was cooled, and organic layer was separated and concentrated under high vacuum. The crude product was purified by silica gel chromatography (0->10% MeOH/CHCl₃) to afford 2-cyclobutyl-5-imidazo[1,2-a]pyridin-6-yl-7-(4-methylphenyl) sulfonylpyrrolo[2,3-d]pyrimidine (CLVIII) (30 mg, 0.068 mmol, 80.7% yield) as a beige solid. ESIMS found for C24H₂IN502S m/z 444.1 (M+1).

### Step 5

To a solution of 2-cyclobutyl-5-imidazo [1,2-a] pyridin-6-yl-7-(4-methylphenyl) sulfonylpyrrolo[2,3-d]pyrimidine (CLVIII) (30 mg, 0.070 mmol) in MeOH (5 mL) was added Cs₂CO₃ (89.5 mg, 0.270 mmol). The reaction mixture was stirred for 2 h at 70° C. Reaction mixture was concentrated, and the residue purified by column chromatography (0->6% 7N NH₃ in MeOH/CHCl₃). The resulting solid was triturated with DCM/hexanes, filtered and dried under high vacuum to produce 2-cyclobutyl-5-(imidazo[1,2-a]pyridin-6-yl)-7H-pyrrolo[2,3-d]pyrimidine (103) (15 mg, 0.052 mmol, 76.6% yield) as a beige solid. ¹H NMR (499 MHz, DMSO-d₆) δppm 1.84-1.95(1 H, m), 1.98-2.13 (1 H, m), 2.28-2.38 (2 H, m), 2.38-2.48 (2 H, m), 3.82 (1 H, quind, J=8.59, 8.59, 8.59, 8.59, 0.96 Hz), 7.57 (1 H, d, J=1.10 Hz), 7.60-7.65 (1 H, m), 7.66-7.71 (1 H, m), 7.99 (2 H, d, J=3.56 Hz), 9.04 (1 H, dd, J=1.64, 0.82 Hz), 9.41 (1H, s), 12.24 (1 H, br s); ESIMS found for C₁₇H₁₅N₅ m/z 290.15 (M+1).

### Example 2

Preparation of 5-(3-(difluoromethyl)imidazo[1,2-a]pyridin-6-yl)-N-(2-fluoro-2-methylpropyl)-7H-pyrrolo[2,3-d]pyrimidin-2-amine (329) is depicted below in Scheme 30.

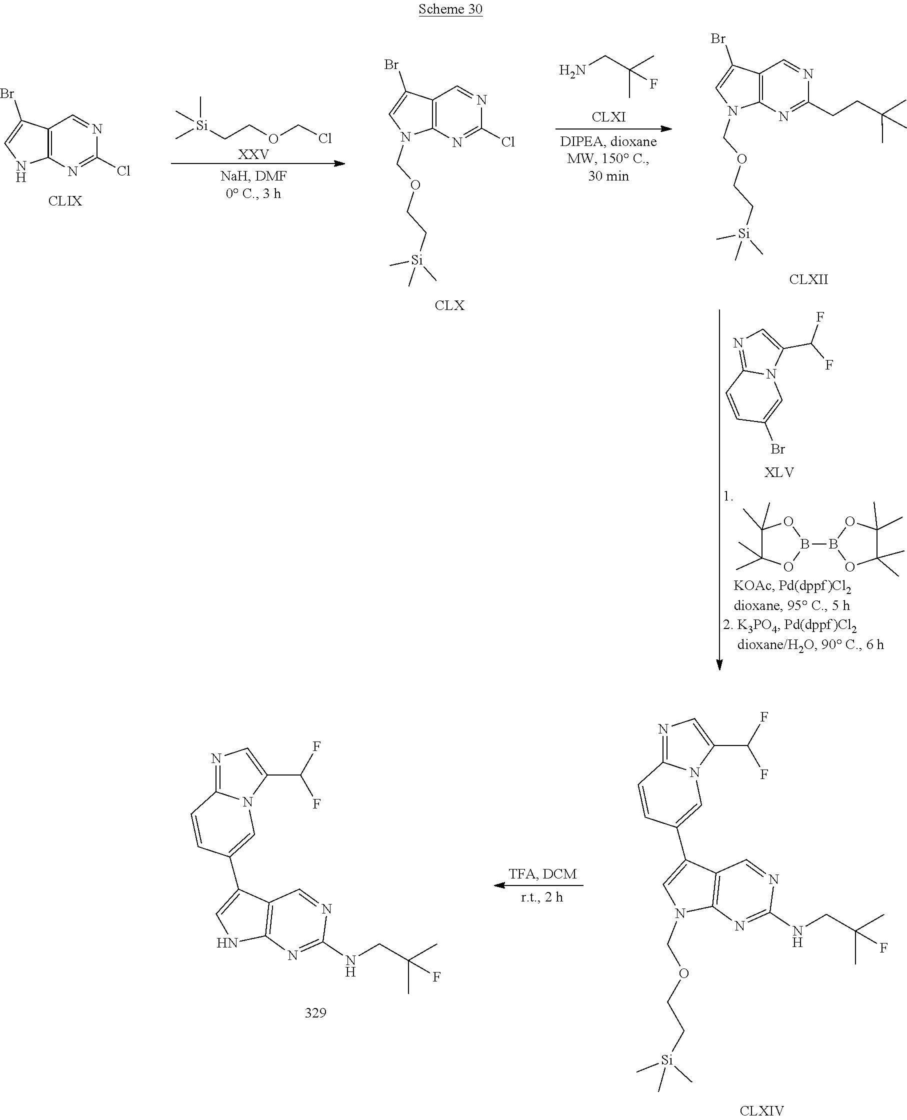

### Step 1

To a stirred solution of 5-bromo-2-chloro-7H-pyrrolo[2,3-d]pyrimidine (CLIX) (commercially available from Combi-Blocks Inc.) (5 g, 21.51 mmol) in DMF (50 mL) was added sodium hydride (1.04 g, 26 mmol) at 0° C. and the mixture was stirred for 15 min. (2-(chloromethoxy)ethyl)trimethylsilane (XXV) (4.mL, 22.6 mmol) was then added and the mixture was stirred for 3 h. The solvents were concentrated, then the residue was dissolved in EtOAc and partitioned between EtOAc/water. The combined organics phase was separated, washed with water and brine, dried over anhydrous Na2SO₄, and then concentrated under high vacuo to obtain 5-bromo-2-chloro-7-((2-(trimethyl silyl)ethoxy)methyl)-7H-pyrrolo [2,3-d]pyrimidine (CLX) (7.57 g, 20.87 mmol, 97.0% yield) as a thick oil which was used in the next step without purification. ESIMS found for C₁₂H₁₇BrClN30Si m/z 362.0 (⁷⁹BrM+H).

### Step 2

A mixture of DIPEA (1.34 mL, 7.71 mmol), 5-bromo-2-chloro-7-((2-(trimethylsilyl)ethoxy)methyl)-7H-pyrrolo[2,3-d]pyrimidine (CLX) (0.63g, 1.74 mmol) and 2-fluoro-2-methylpropan-1-amine (CLXI) (670 mg, 5.25 mmol) in 1,4-dioxane (3 mL) was heated to 150° C. for 3 h in microwave. Reaction was concentrated and the residue purified via column chromatography. (0→2% MeOH/CHCl₃) to yield 5-bromo-N-(2-fluoro-2-methylpropyl)-7-(2-trimethylsilylethoxymethyl) pyrrolo [2,3-d]pyrimidin-2-amine (CLXII) (310 mg, 0.743 mmol, 42.8% yield) as a white solid. ESIMS found for C₁₆H₂₆BrFN₄OSi m/z 417.1 (M+H).

### Step 3-4

A mixture of 6-bromo-3-(difluoromethyl)imidazo[1,2-a]pyridine (XLV) (80 mg, 0.320 mmol), bis(pinacolato)diboron (120 mg, 0.480 mmol), KOAc (90 mg, 0.960 mmol) and Pd(dppf)Cl₂ (20 mg, 0.030 mmol) was taken in 1,4-dioxane (2 mL) and N₂ gas was bubbled into the mixture for 5 min and then mixture was heated to 95° C. for 5 h.

To the mixture was added 5-bromo-N-(2-fluoro-2-methylpropyl)-7-(2-trimethylsilylethoxymethyppyrrolo[2,3-d]pyrimidin-2-amine (CLXII) (110 mg, 0.260 mmol), Pd(dppf)Cl₂ (20 mg, 0.030 mmol) and 2 M aqueous solution of K3PO₄ (0.33 mL, 0.660 mmol). N2 was bubbled into the mixture for 5 min and the mixture was heated to 90° C. for 6 h. The reaction was filtered through ^{Celite}® and concentrated under high vacuum. The residue was purified via column chromatography (0→3% 7 N NH₃ in MeOH/CHCl₃) to produce 5-(3-(difluoromethyl)imidazo[1,2-a]pyridin-6-yl)-N-(2-fluoro-2-methylpropyl)-7-42-(trimethylsilyl) ethoxy)methyl)-7H-pyrrolo[2,3-d]pyrimidin-2-amine (CLXIV) (62 mg, 0.123 mmol, 47.9% yield) as an amber viscous solid. ESIMS found for C₂₄H₃₁F₃N₆OSi m/z 505.3 (M+H).

### Step 5

To a stirred solution of 5-(3-(difluoromethyl)imidazo[1,2-a]pyridin-6-yl)-N-(2-fluoro-2-methylpropyl)-7-42-(trimethylsilyl) ethoxy)methyl)-7H-pyrrolo [2,3-d] pyrimidin-2-amine (CLXIV) ((70 mg, 0.150 mmol) in DCM (1 mL) was added TFA (0.55 mL, 7.09 mmol) and the mixture was stirred for 2 h. The reaction was concentrated and basified with 7 N NH₃ solution in MeOH. The solution was concentrated and purified via column chromatography (0→8% 7 N NH₃ in MeOH/CHCl₃) to give 5-(3-(difluoromethyl)imidazo[1,2-a]pyridin-6-yl)-N-(2-fluoro-2-methylpropyl)-7H-pyrrolo[2,3-d]pyrimidin-2-amine (329) (4 mg, 0.011 mmol, 8.7% yield) as a white solid. ¹1-1 NMR (499 MHz, DMSO-d₆) δppm 1.35 (6 H, d, J=21.40 Hz), 3.60 (2 H, dd, J=19.16, 6.30 Hz), 6.94 (1 H, br t, J=6.16 Hz), 7.68 (1 H, t, J=53.60 Hz), 7.63 (1 H, d, J=2.19 Hz), 7.75-7.78 (1 H, m), 7.81-7.85 (1 H, m), 7.91-7.95 (1 H, m), 8.62 (1 H, s), 8.84(1 H, s), 11.64 (1 H, br s); ESIMS found for C₁₈1-1₁₇F₃N₆ m/z 375.2 (M+1).

### Example 3

Preparation of 5-(3-chloroimidazo[1,2-b]pyridazin-6-yl)-N-(2-fluoro-2-methylpropyl)-7H-pyrrolo[2,3-d]pyrimidin-2-amine (952) is depicted below in Scheme 31.

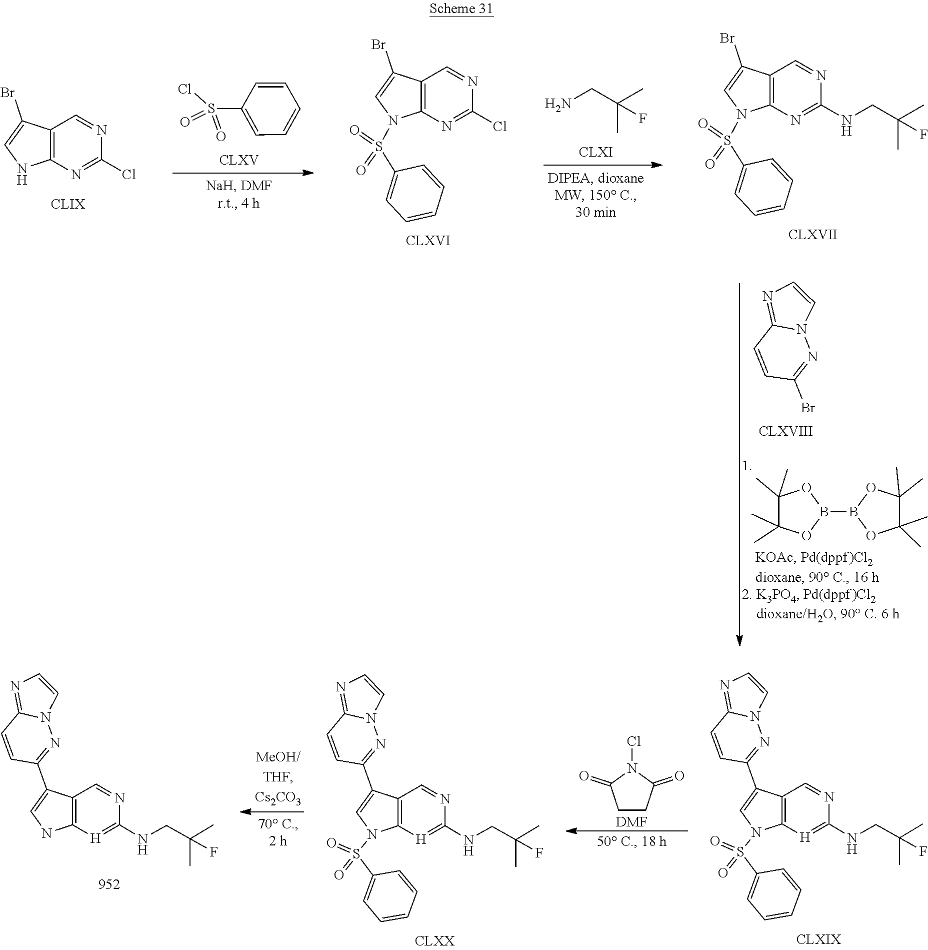

### Steps 1-4

Steps were performed using procedures shown in Example 1, Scheme 29, Step 3 and Example 2, Scheme 30, steps 2-4.

### Step 5

To a stirring solution of 7-(benzenesulfonyl)-N-(2-fluoro-2-methylpropyl)-5-imidazo[1,2-b]pyridazin-6-ylpyrrolo[2,3-d]pyrimidin-2-amine (CLXIX) (0.1 g, 0.210 mmol) in DMF (2 mL) was added N-chlorosuccinimide (0.03 g, 0.240 mmol). The reaction mixture was allowed to stir at 50° C. for 18 h. The mixture was concentrated and purified via column chromatography (0→4% 7 N NH₃ in MeOH/CHCl₃) (4 g of silica gel) to yield 7-(benzene sulfonyl)-5-(3-chloroimidazo [1,2-b] pyridazin-6-yl)-N-(2-fluoro-2-methylpropyl)pyrrolo[2,3-d]pyrimidin-2-amine (CLXX) (37 mg, 0.074 mmol, 34.5% yield) as an off-white solid. ESIMS found for C₂₂H₁₉ClFN₇O₂S m/z 500.1 (M+H).

### Step 6

The deprotection of 7-(benzenesulfonyl)-5-(3-chloroimidazo[1,2-b]pyridazin-6-yl)-N-(2-fluoro-2-methylpropyl)pyrrolo[2,3-d]pyrimidin-2-amine (CLXX) followed the procedure in Scheme 18, step 5 to obtain 5-(3-chloroimidazo[1,2-b]pyridazin-6-yl)-N-(2-fluoro-2-methylpropyl)-7H-pyrrolo[2,3-d]pyrimidin-2-amine (952) (12 mg, 0.033 mmol, 45.1% yield) as a white solid. ¹H NMR (499 MHz, DMSO-d₆) δppm 1.35 (6 H, d, J=21.40 Hz), 3.61 (2 H, br dd, J=19.16, 6.57 Hz), 7.03 (1 H, br s), 7.82 (1 H, s), 7.85 (1 H, d, J=9.86 Hz), 8.13 (1 H, d, J=9.31 Hz), 8.16 (1 H, s), 9.33 (1 H, s), 11.91 (1 H, br s); ESIMS found for C₁₆H₁₅ClFN₇ m/z 360.1 (M+1).

### Example 4

Preparation of (5-(4-((cyclopropylmethyl)amino)-7H-pyrrolo [2,3-d] pyrimidin-5-yl)pyrazolo [1,5-a] pyridin-3-yl)(4-methylpipe razin-1-yl)methanone (1244) is depicted below in Scheme 32.

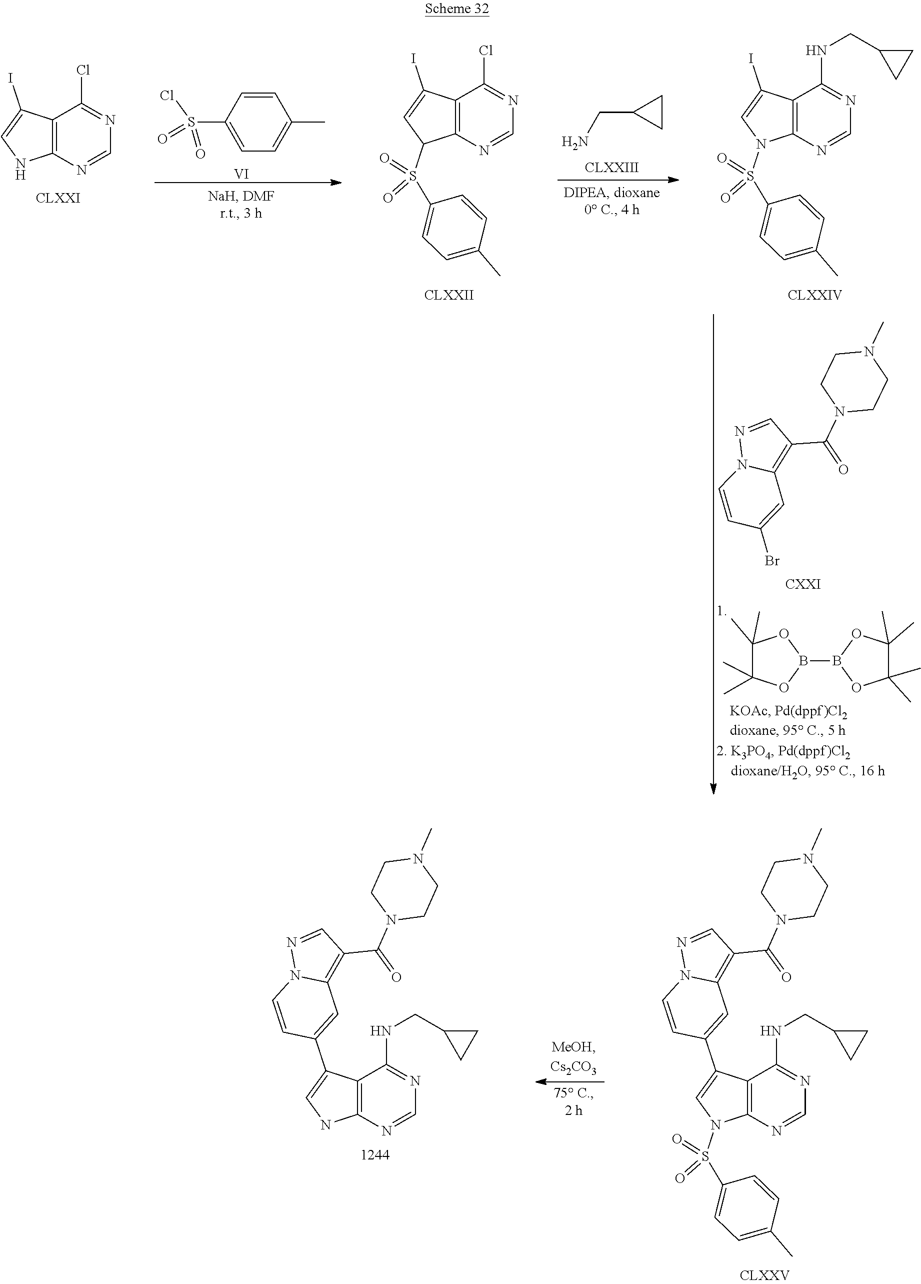

### Step 1

To a stirred solution of 4-chloro-5-iodo-7H-pyrrolo[2,3-d]pyrimidine (CLXXI) (commercially available from Combi-Blocks Inc.) (2 g, 7.16 mmol) in DMF (25 mL) was added sodium hydride (0.35 g, 8.63 mmol) at 0° C. and the mixture was stirred for 30 min. 4-Methylbenzenesulfonyl chloride (VI) was added (1.5 g, 7.87 mmol) in 3 ml of DMF and the mixture was stirred at room temperature for 3 h. The reaction mixture was added to water (100 mL) and stirred for 30 min. The resulting solids were collected by filtration and dried under high vacuo to obtain 4-chloro-5-iodo-7-tosyl-7H-pyrrolo[2,3-d]pyrimidine (CLXXII) (3 g, 6.92 mmol, 96.7% yield) as an off-white solid. ESIMS found for C₁₃H₉ClIN₃O₂S m/z 433.9 (M+H).

### Step 2

To a stirred solution of DIPEA (0.36 mL, 2.07 mmol) in 1,4-Dioxane (2 mL) was added 4-chloro-5-iodo-7-tosyl-7H-pyrrolo[2,3-d]pyrimidine (CLXXII) (300 mg, 0.690 mmol) at 0° C. and the mixture was stirred for 15 min. Cyclopropylmethanamine (CLXXIII) (70 mg, 1.04 mmol) in DMF (5 mL) was then added and the mixture was stirred at 0° C. for 4 h. The reaction mixture was added to water, (200 mL) and stirred for 1 h. The resulting solid was collected by filtration and dried under high vacuo to obtain N-(cyclopropylmethyl)-5-iodo-7-tosyl-7H-pyrrolo[2,3-d]pyrimidin-4-amine (CLXXIV) (257 mg, 0.549 mmol, 79.3% yield) as an off-white solid. ESIMS found for C₁₇H₁₇IN₄O₂S m/z 469.1 (M+H).

### Step 3-4

A mixture of N-(cyclopropylmethyl)-5-iodo-7-tosyl-7H-pyrrolo [2,3-d] pyrimidin-4-amine (CLXXIV) (170 mg, 0.360 mmol), bis(pinacolato)diboron (170 mg, 0.680 mmol), KOAc (130 mg, 1.36 mmol) and Pd(dppf)Cl₂ (40 mg, 0.040 mmol) in 1,4-dioxane (1.5 mL) was purged with N₂ gas for 5 min. The reaction was heated to 95° C. for 5 h.

To the mixture was added (5-bromopyrazolo[1,5-a]pyridin-3-yl)(4-methylpiperazin-1-yl)methanone (CXXI) (150 mg, 0.450 mmol) in dioxane (0.5 mL), Pd(dppf)Cl₂ (40 mg, 0.040 mmol) and 2 M aqueous solution of K₃PO₄ (0.45 mL, 0.900 mmol). The solution was purged with N₂ for 5 min and the mixture was heated to 95° C. for 16 h. The organic layer was separated, absorbed on silica gel, and purified by ISCO (10% EtOAc/hexanes →100% EtOAc) to obtain (5-(4-((cyclopropylmethyl)amino)-7-(phenyl sulfonyl)-7H-pyrrolo [2,3-d]pyrimidin-5-yl)pyrazolo[1,5-a]pyridin-3-yl)(4-methylpiperazin-1-yl) methanone (CLXXV) (204 mg, 0.349 mmol, 97.3% yield) as a grey solid. ESIMS found for C30I⁻132N803S m/z 585.1 (M+H).

### Step 5

A suspension of Cs2CO₃ (330 mg, 1.03 mmol) and (544-((cyclopropylmethyDamino)-7-(phenyl sulfonyl)-7H-pyrrolo [2,3-d] pyrimidin-5-yl)pyrazolo [1,5-a]pyridin-3-yl)(4-methylpiperazin-1-yl)methanone (CLXXV) (200 mg, 0.340 mmol) in a mixture of THF (1 mL) and MeOH (1 mL) was heated at 70° C. for 16 h. The reaction mixture was cooled to room temperature and concentrated under vacuum. The residue was dissolved in a mixture of CHCl₃/MeOH and then purified by preparative TLC (6% 7 N NH₃ MeOH in CHCl₃) to afford (5-(4-((Cyclopropylmethyl)amino)-7H-pyrrolo [2,3-d] pyrimidin-5-yl)pyrazolo [1,5-a] pyridin-3-yl)(4-methylpiperazin-1-yl)methanone (1244) (35 mg, 0.081 mmol, 23.8% yield) as an off-white solid. ¹H NMR (499 MHz, DMSO-d₆) δppm 0.18-0.26 (2 H, m), 0.34-0.42 (2 H, m), 1.08-1.18 (1 H, m), 2.20 (3 H, s), 2.33 (4 H, t, J=4.79 Hz), 3.30-3.33 (2 H, m), 3.62-3.70 (4 H, m), 5.94 (1H, t, J=5.34 Hz), 7.18 (1 H, dd, J=7.12, 1.92 Hz), 7.51 (1 H, d, J=2.19 Hz), 7.86 (1 H, d, J=1.09 Hz), 8.20 (1 H, s), 8.26 (1 H, s), 8.78-8.85 (1 H, m), 12.01 (1 H, br s); ESIMS found for C₂₃H₂₆N₈O m/z 431.2 (M+1).

### Example 5

Preparation of 5-(2-amino-7H-pyrrolo [2,3-d]pyrimidin-5-yl)-N-(1-methylpiperidin-4-yl)pyrazolo [1,5-a]pyridine-3-carboxamide (1511) is depicted below in Scheme 33.

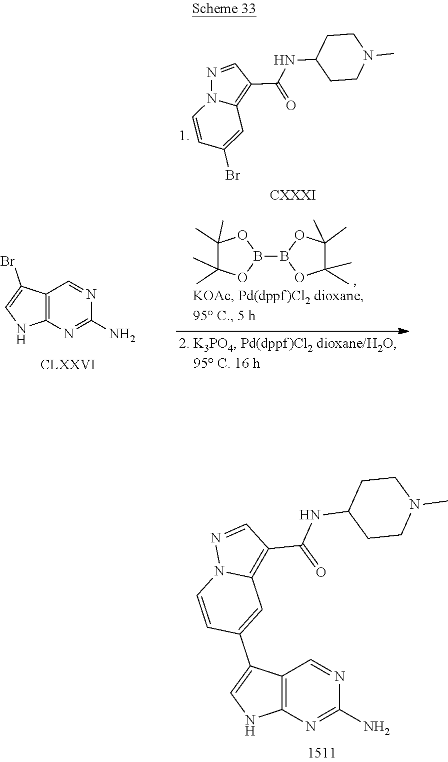

### Steps 1-2

A mixture of 5-bromo-N-(1-methylpiperidin-4-yl)pyrazolo[1,5-a]pyridine-3-carboxamide (CXXXI) (0.2g, 0.590 mmol), bis(pinacolato)diboron (0.22 g, 0.880 mmol), KOAc (0.17 g, 1.76 mmol) and Pd(dppf)Cl₂ (0.04 g, 0.050 mmol) was dissolved in 1,4-dioxane (2.5 mL). N₂ gas was then bubbled into the mixture for 5 min before heating the mixture to 95° C. for 5 h.

To the mixture was added 5-bromo-7h-pyrrolo[2,3-d]pyrimidin-2-amine (CLXXVI) (commercially available from Advanced ChemBlocks Inc.) (0.1 g, 0.470 mmol), Pd(dppf)Cl₂ (0.04 g, 0.050 mmol) and 2 M aqueous solution of K3PO₄ (0.77 mL, 1.54 mmol). N2 was bubbled into the mixture for 5 min before heating the mixture to 95° C. for 16 h. The organic layer was separated, absorbed on silica gel, and purified by ISCO (1→10% 7 N NH₃ MeOH/CHCl₃) followed by preparative TLC (7% of 7 N NH₃ MeOH/CHCl₃) to obtain 5-(2-amino-7H-pyrrolo [2,3-d]pyrimidin-5-yl)-N-(1-methylpipe ridin-4-yl)pyrazolo [1,5-a] pyridine-3-carboxamide 1511 (10.0 mg, 0.026 mmol, 5.5% yield) as an off-white solid. ¹H NMR (500 MHz, DMSO-d₆) δppm 1.50-1.65 (2 H, m), 1.77-1.85 (2 H, m), 1.95 (2 H, td, J=11.73, 2.06 Hz), 2.18 (3 H, s), 2.79 (2 H, br d, J=11.53 Hz), 3.71-3.85 (1 H, m), 6.33 (2 H, s), 7.41 (1 H, dd, J=7.27, 2.06 Hz), 7.79 (1 H, s), 7.93 (1 H, d, J=7.96 Hz), 8.43 (1 H, d, J=1.37 Hz), 8.54 (1 H, s), 8.69 (1 H, dd, J=7.14, 0.82 Hz), 8.87 (1 H, s), 11.64 (1 H, br s); ESIMS found for C20H₂₂N80 m/z 391.1 (M+1).

### Example 6

Preparation of N-(pyridin-4-yl)-5-(quinolin-6-yl)-7H-pyrrolo [2,3-d]pyrimidin-2-amine (1892) is depicted below in Scheme 34.

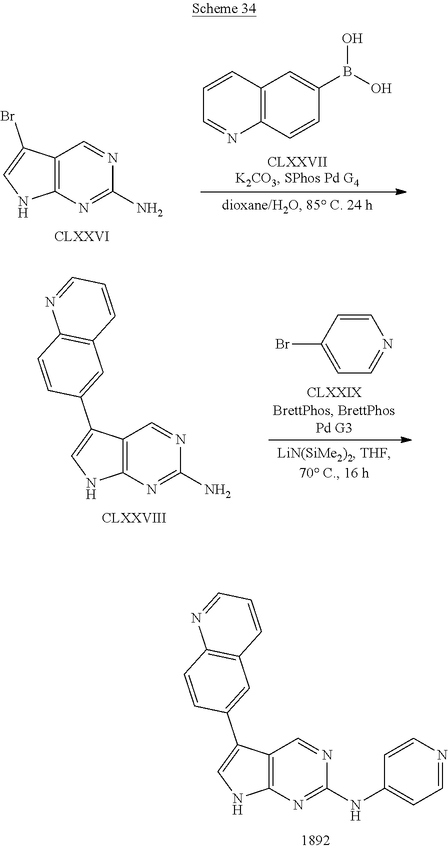 [SMILES: Nc1ncc2c(Br)c[nH]c2n1 ; OB(O)c1ccc2ncccc2c1 ; Nc1ncc2c(-c3ccc4ncccc4c3)c[nH]c2n1 ; Brc1ccncc1 ; c1cnc2ccc(-c3c[nH]c4nc(Nc5ccncc5)ncc34)cc2c1]

### Step 1

To a mixture of quinoline-6-boronic acid (CLXXVII) (302.06 mg, 1.75 mmol), 5-bromo-7h-pyrrolo[2,3-d]pyrimidin-2-amine (CLXXVI) (310 mg, 1.46 mmol) and SPhos Pd G4 (57.87 mg, 0.070 mmol) was in 1,4-dioxane (6.2 mL) was added a 2 M aqueous solution of K2CO₃ (1.46 mL, 2.91 mmol). N₂ gas was bubbled into the mixture for 1 min and the mixture stirred at 85° C. in a sealed vial for 1 day. The reaction mixture was cooled to room temperature, concentrated and the crude product purified by silica gel chromatography (100% hexanes →100% EtOAc →10% MeOH/Et0Ac). The fractions were concentrated and dried under high vacuo to afford 5-(quinolin-6-yl)-7H-pyrrolo[2,3-d]pyrimidin-2-amine (CLXXVIII) (70 mg, 0.268 mmol, 18.4% yield) as a brown solid. ESIMS found for CisHiiN₅ m/z 262.1 (M+1).

### Step 2

A mixture of 5-quinolin-6-yl-7H-pyrrolo [2,3-d]pyrimidin-2-amine (CLXXVIII) (82 mg, 0.310 mmol) 4-bromo-pyridine HC1 (CLXXIX) (73.24 mg, 0.380 mmol), BrettPhos (8.42 mg, 0.020 mmol), and BrettPhos Pd G3 (14.22 mg, 0.020 mmol) was purged with N2. Lithium bis(trimethylsilyl) amide solution (1 M in THF) (0.94 mL, 0.940 mmol) was added to the mixture and heated in a sealed tube at 70° C. under the N2 for 16 h. The reaction mixture was cooled to room temperature and concentrated. The crude product was purified by silica gel chromatography (0→10% MeOH/CHCl₃). The fractions containing the product were concentrated and the residue triturated in ether. The resulting solid was filtered and dried under high vacuo to afford N-(pyridin-4-yl)-5-(quinolin-6-yl)-7H-pyrrolo[2,3-d]pyrimidin-2-amine (1892) (50 mg, 0.148 mmol, 47.1% yield) as a beige solid. ¹H NMR (499 MHz, DMSO-d₆) δppm 7.54 (1 H, dd, J=8.37, 4.25 Hz), 7.93 (2 H, d, J=6.31 Hz), 8.00 (1 H, d, J=2.47 Hz), 8.04 (1 H, d, J=8.78 Hz), 8.22 (1 H, dd, J=8.92, 2.06 Hz), 8.38 (2 H, d, J=6.31 Hz), 8.41 (1 H, d, J=1.65 Hz), 8.46-8.52 (1 H, m), 8.85 (1 H, dd, J=4.12, 1.65 Hz), 9.44 (1 H, s), 10.16 (1 H, s), 12.16 (1 H, s); ESIMS found for C₂₀H₁₄N₆ m/z 339.1 (M+1).

### Example 7

Preparation of 6,6′-(7H-pyrrolo[2,3-d]pyrimidine-2,5-diyl)diquinoline (1896) is depicted below in Scheme 35.

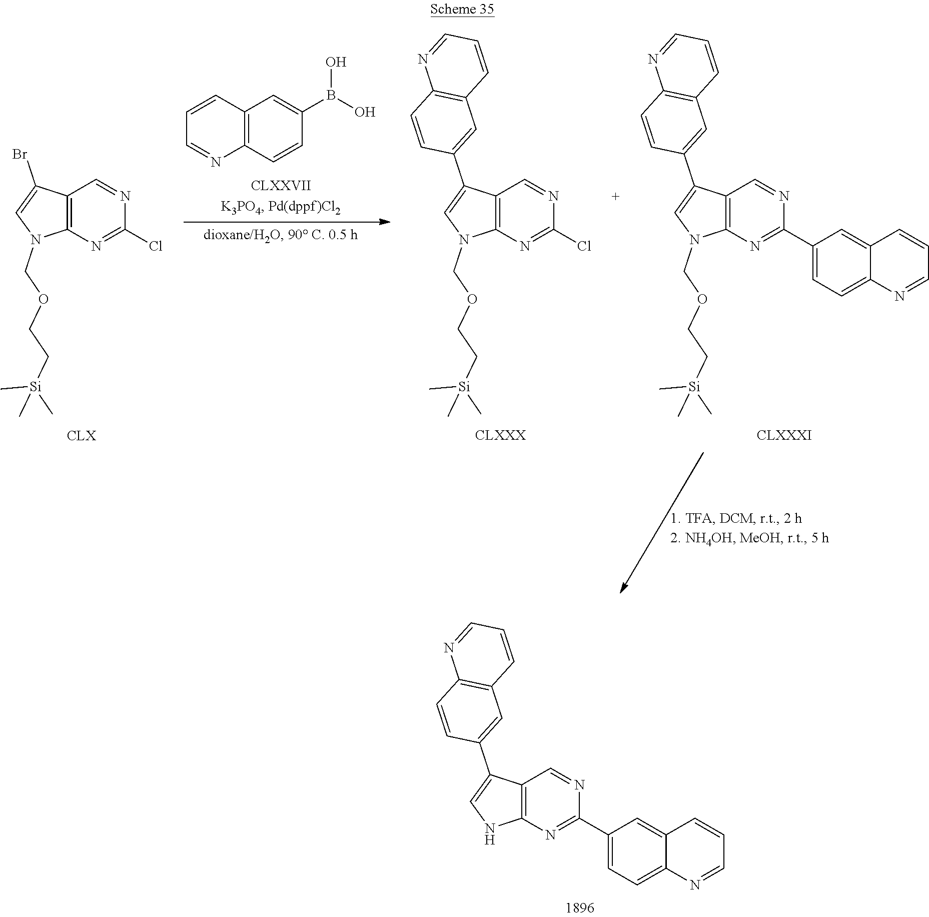 [SMILES: C[Si](C)(C)CCOCn1cc(Br)c2cnc(Cl)nc21 ; OB(O)c1ccc2ncccc2c1 ; C[Si](C)(C)CCOCn1cc(-c2ccc3ncccc3c2)c2cnc(Cl)nc21 ; C[Si](C)(C)CCOCn1cc(-c2ccc3ncccc3c2)c2cnc(-c3ccc4ncccc4c3)nc21 ; c1cnc2ccc(-c3ncc4c(-c5ccc6ncccc6c5)c[nH]c4n3)cc2c1]

### Step 1

A mixture of 2-[(5-bromo-2-chloropyrrolo[2,3-d]pyrimidin-7-yl)methoxy]ethyl-trimethylsilane (CLX) (629 mg, 1.73 mmol), quinoline-6-boronic acid (CLXXVII) (300 mg, 1.73 mmol), Pd(dppf)Cl₂ (60 mg, 0.070 mmol), and 2 M aqueous solution of K₃PO₄ (2 mL, 4 mmol) in 1,4-dioxane (10 mL) was purged with N₂ gas for 5 min. The mixture was heated with microwave irradiation at 90° C. for 30 min. The reaction mixture was cooled, and organic layer was separated, concentrated, resulting crude product was purified by silica gel chromatography (0->100% EtOAc/hexanes). The pure fractions were combined and concentrated to afford two products, 2-[(2-chloro-5-quinolin-6-ylpyrrolo[2,3-d]pyrimidin-7-yl)methoxy]ethyl-trimethylsilane (CLXXX) (400 mg, 0.973 mmol, 56.1% yield) as a beige solid and 24[[2,5-di (quinolin-6-yl)pyrrolo [2,3-d]pyrimidin-7-yl]methoxy]ethyl-trimethylsilane (CLXXXI) (50 mg, 0.099 mmol, 5.7% yield) as a beige solid. ESIMS found for C211⁻123ClN40Si m/z 411.0 (M+1) and ESIMS found for C₃ₒH₂₉N₅OSi m/z 504.1 (M+1).

### Step 2

To a solution of 2-[[2,5-di(quinolin-6-yl)pyrrolo[2,3-d]pyrimidin-7-yl]mthoxy]ethyl-trimethylsilane (CLXXXI) (50 mg, 0.100 mmol) in DCM (1 mL), was added TFA (0.2 mL, 2.6 mmol). The reaction mixture was stirred for 2 h at room temperature. LCMS showed incomplete deprotection. MS showed presence of N-methyl alcohol. Reaction mixture was concentrated, and then dissolved in 30% solution of NH₄OH in MeOH (0.5 mL) and MeOH (2 mL). The reaction mixture was stirred for 5 h at room temperature. LCMS showed complete deprotection. Reaction mixture was concentrated, the residue was triturated with MeOH resulting solid was filtered and dried under high vacuum to produce 6,6′-(7H-pyrrolo[2,3-d]pyrimidine-2,5-diyl)diquinoline (1896) (20 mg, 0.054 mmol, 54.0% yield) as a beige solid. ¹1-1 NMR (499 MHz, DMSO-d₆) δ ppm 7.66 (2 H, dt, J=8.44, 4.43 Hz), 8.13 (1 H, d, J=8.78 Hz), 8.20 (1 H, d, J=8.78 Hz), 8.34 (1 H, d, J=2.47 Hz), 8.36 (1 H, dd, J=9.06, 1.92 Hz), 8.57 (1 H, d, J=1.37 Hz), 8.64 (1 H, br d, J=8.23 Hz), 8.67 (1 H, br d, J=7.96 Hz), 8.90 (1 H, dd, J=8.78, 1.92 Hz), 8.93-8.97 (1 H, m), 9.00 (1 H, dd, J=4.12, 1.37 Hz), 9.13 (1 H, d, J=1.65 Hz), 9.81 (1 H, s), 12.69 (1 H, br s); ESIMS found for C24H₁₅N5 m/z 374.1 (M+1).

### Example 8

Preparation of 6-(4-(1-methyl-1H-pyrazol-4-yl)-7H-pyrrolo[2,3-d]pyrimidin-5-yl)quinoline (1897) is depicted below in Scheme 36.

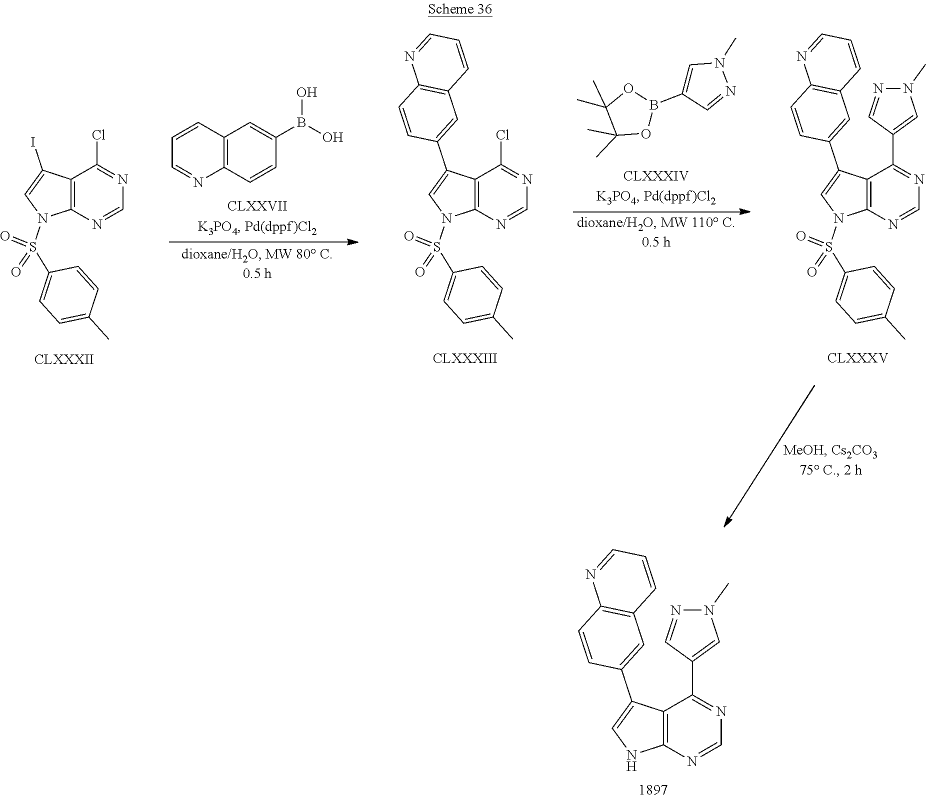

### Step 1

A mixture of 4-chloro-5-iodo-7-(4-methylphenyl)sulfonylpyrrolo [2,3-d]pyrimidine (CLXXXII) (400 mg, 0.920 mmol), quinoline-6-boronic acid (CLXXVII) (176 mg, 1.02 mmol), 2 M aqueous solution of K₃PO₄ (1.4 mL, 2.8 mmol) and Pd(dppf)Cl₂ (40 mg, 0.050 mmol) in 1,4-dioxane (8 mL) was purged with N₂ gas for 5 min. The mixture was heated with microwave irradiation for 30 min at 80° C. The reaction mixture was cooled, and organic layer was separated and concentrated. The crude product was purified by silica gel chromatography (0→10% MeOH/CHCl₃). The pure fractions were combined and concentrated to afford a mixture of 6-[4-chloro-7-(4-methylphenyl)sulfonylpyrrolo [2,3-d] pyrimidin-5-yl]quinoline (CLXXXIII) (370 mg, 0.851 mmol, 92.2% yield) as a beige solid. ESIMS found for C22H₁₅ClN402S m/z 435.1 (M+H).

### Step 2

A mixture of 6-[4-chloro-7-(4-methylphenyl)sulfonylpyrrolo [2,3-d]pyrimidin-5-yl]quinoline (CLXXXIII) (73 mg, 0.170 mmol), 1-methyl-4-(4,4,5,5-tetramethyl-1,3,2-dioxaborolan-2-yl)pyrazole (CLXXXIV) (38 mg, 0.180 mmol), 2 M aqueous solution of K₃PO₄ (0.25 mL, 0.500 mmol) and Pd(dppf)Cl₂ (7 mg, 0.010 mmol) in 1,4-dioxane (3 mL) was purged with N₂ gas for 5 min. The mixture was heated with microwave irradiation for 30 min at 110° C. The reaction mixture was cooled, and organic layer was separated, concentrated to afford crude 6-[7-(4-methylphenyl)sulfonyl-4-(1-methylpyrazol-4-yl)pyrrolo [2,3-d]pyrimidin-5-yl]quinoline (CLXXXV) (85 mg, 0.177 mmol, 105.4% yield) as a beige solid used as such for the next step without further purification. ESIMS found for C₂₆H₂₀N₆O₂S m/z 481.1 (M+H).

### Step 3

A suspension of Cs2CO₃ (300 mg, 0.920 mmol) and 6-[7-(4-methylphenyl)sulfonyl-4-(1-methylpyrazol-4-yl)pyrrolo [2,3-d] pyrimidin-5-yl] quinoline (CLXXXV) (85 mg, 0.180 mmol) in MeOH (1 mL) was heated at 75° C. for 2 h. The reaction mixture was cooled to room temperature and concentrated. The crude was then purified by flash column chromatography (0→5% MeOH/CHCl₃). The desired fractions were concentrated to dryness in vacuo and the residue triturated in ether. The resulting solid was filtered and dried under high vacuo to afford 6-(4-(1-Methyl-1H-pyrazol-4-yl)-7H-pyrrolo [2,3-d] pyrimidin-5-yl)quinoline (1897) (10 mg, 0.031 mmol, 17.3% yield) as a white solid. ¹H NMR (499 MHz, DMSO-d₆) δppm 3.50 (3 H, s), 7.13 (1 H, s), 7.49 (1 H, s), 7.52-7.58 (2 H, m), 7.78 (1 H, d, J=1.65 Hz), 7.83 (1 H, s), 7.91 (1 H, d, J=8.51 Hz), 8.23 (1 H, d, J=7.41 Hz), 8.77 (1 H, s), 8.90 (1 H, dd, J=4.25, 1.78 Hz), 12.47 (1 H, br s); ESIMS found for C₁₉H₁₄N₆ m/z 327.1 (M+1).

### Example 9

Preparation of N-(2-(4-methylpipe razin-1-yl)pyridin-4-yl)-5-(3-(pyridin-3-yl) pyrazolo [1,5-a] pyridin-5-yl)-7H-pyrrolo [2,3-d] pyrimidin-2-amine (2031) is depicted below in Scheme 37.

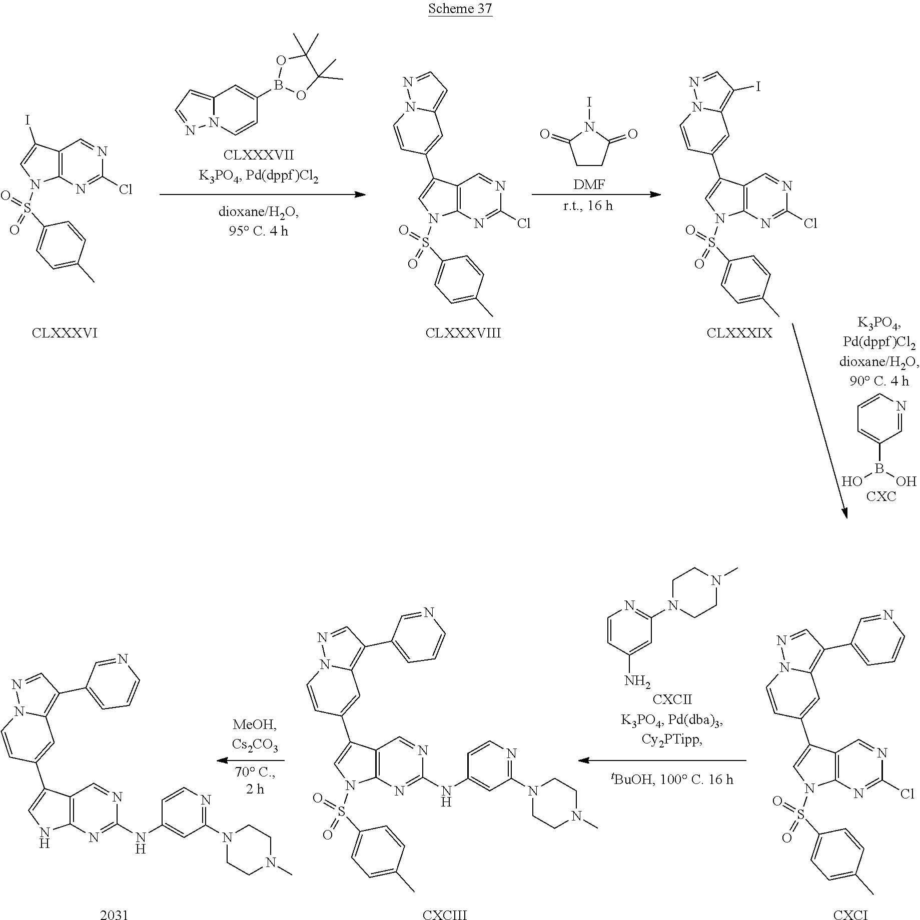

### Step 1

Performed using procedure shown in Example 7, Scheme 35, Step 1 to yield 2-chloro -5-(pyrazolo [1,5-a] pyridin-5-yl)-7-tosyl-7H-pyrrolo [2,3-d]pyrimidine (CLXXXVIII) (287 mg, 0.677 mmol, 58.7% yield) as an off-white solid. ESIMS found for C₂₀H₁₄ClN₅O₂S m/z 424.0 (M+H).

### Step 2

Performed using procedure shown in Example 1, Scheme 29, Step 2 to obtain 2-chloro-5-(3-iodopyrazolo [1,5-a] pyridin-5-yl)-7-tosyl-7H-pyrrolo [2,3-d]pyrimidine (CLXXXIX) (179 mg, 0.326 mmol, 48.1% yield) as a beige solid. ESIMS found for C₂₀H₁₃ClIN₅O₂S m/z 549.8 (M+H).

### Step 3

Performed using procedure shown in Example 7, Scheme 35, Step 1 to produce 2-chloro-5-(3-(pyridin-3-yl)pyrazolo[1,5-a]pyridin-5-yl)-7-tosyl-7H-pyrrolo[2,3-d]pyrimidine (CXCI) (56 mg, 0.112 mmol, 35.1% yield) as an off-white solid. ESIMS found for C₂₅H₁₇ClN₆O₂S m/z 501.0 (M+H).

### Step 4

A mixture of 2-chloro-7-(4-methylphenyl)sulfonyl-5-(3-pyridin-3-ylpyrazolo [1,5-a] pyridin-5-yl)pyrrolo [2,3-d]pyrimidine (CXCI) (97 mg, 0.190 mmol), 2-(4-methylpiperazin-1-yl)pyridin-4-amine (CXCII) (57.3 mg, 0.300 mmol), Pd₂(dba)₃ (18.2 mg, 0.020 mmol), Cy₂PTipp (15.6 mg, 0.030 mmol) and K2CO₃ (80 mg, 0.580 mmol) was taken in ^{t}BuOH (2 mL). N₂ gas was bubbled into the mixture for 2 min before heating to 100° C. for 16 h. The reaction mixture was absorbed on silica gel and was purified by ISCO (0→10% 7 N NH₃ MeOH/CHCl₃) followed by preparative TLC (7% of 7 N NH₃ MeOH/CHCl₃) to obtain N-(2-(4-methylpiperazin-1-yl)pyridin-4-yl)-5-(3-(pyridin-3-yl)pyrazolo [1,5-a] pyridin-5-yl)-7-tosyl-7H-pyrrolo [2,3-d]pyrimidin-2-amine (CXCIII) (70 mg, 0.107 mmol, 55.0% yield) as a yellow solid. ESIMS found for C35H₃₂N1002S m/z 657.15 (M+H).

### Step 5

Performed using procedure shown in Example 8, Scheme 36, Step 3 to produce N-(2-(4-methylpipe razin-1-yl)pyridin-4-yl)-5-(3-(pyridin-3-yl)pyrazolo [1,5-a]pyridin-5-yl)-7H-pyrrolo[2,3-d]pyrimidin-2-amine (2031) (7.8 mg, 0.016 mmol, 14.6% yield) as an off-white solid. ¹H NMR (499 MHz, DMSO-d₆) δppm 2.22 (3 H, s), 2.38-2.45 (4 H, m), 3.37-3.43 (4 H, m), 6.83 (1 H, d, J=9.03 Hz), 7.41 (1 H, dd, J=7.26, 1.78 Hz), 7.47-7.54 (1 H, m), 7.93 (1 H, s), 7.95 (1 H, dd, J=9.03, 2.74 Hz), 8.12 (1 H, d, J=0.82 Hz), 8.16-8.23 (1 H, m), 8.46 (1 H, s), 8.49 (1 H, dd, J=4.79, 1.51 Hz), 8.55 (1 H, d, J=2.74 Hz), 8.74-8.79 (1 H, m), 9.00-9.05 (1 H, m), 9.08 (1 H, s), 9.16 (1 H, s), 11.92 (1 H, s); ESIMS found for C₂₈H₂₆N₁₀ m/z 503.15 (M+1).

### Example 10

Preparation of (R)-2-((5-(imidazo [1,2-a]pyridin-6-yl)-2-((1,1, 1-trifluoropropan-2-yl)amino)-7H-pyrrolo [2,3-d]pyrimidin-4-yl)oxy)ethan-1-ol (2178) is depicted below in Scheme 38.

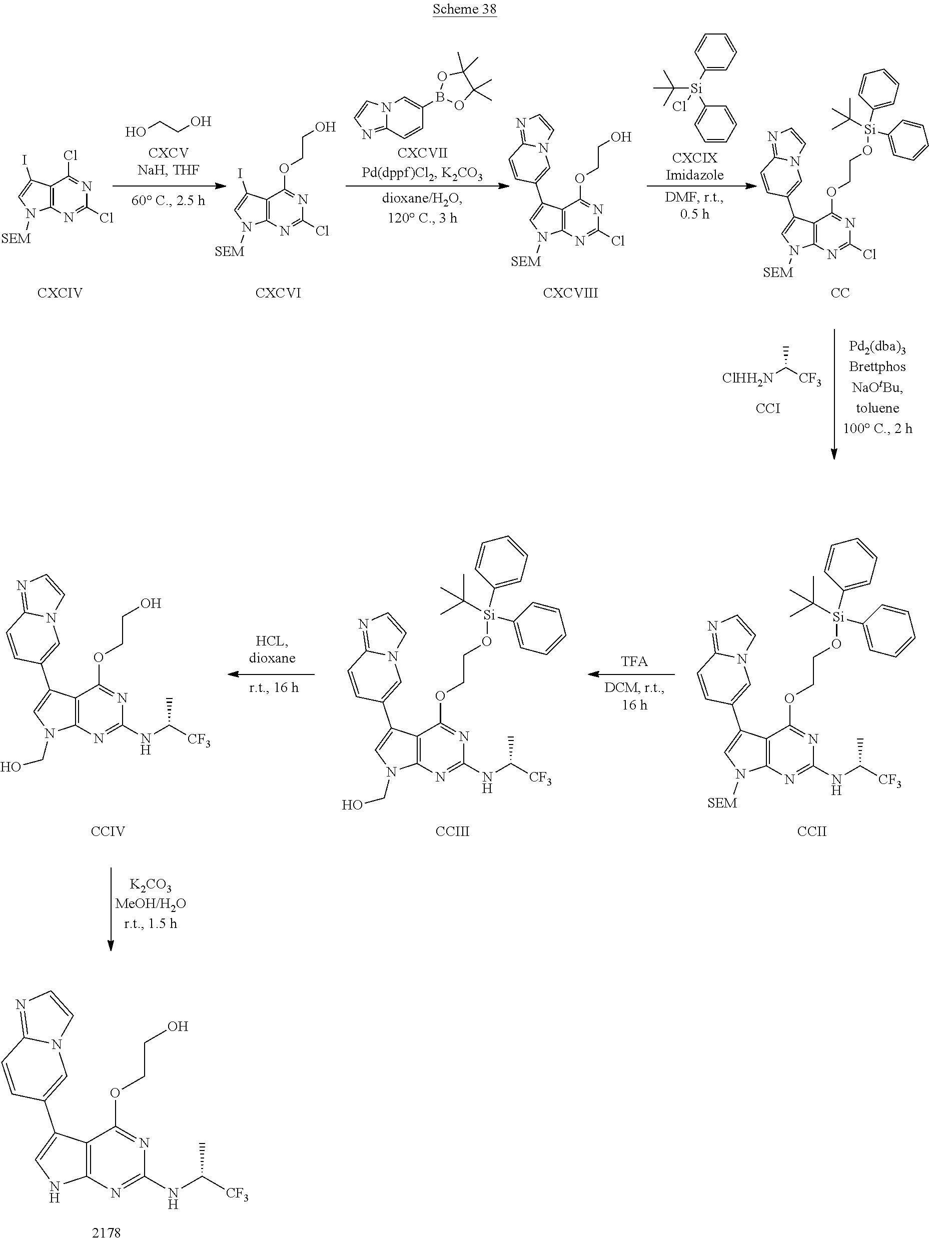

### Step 1

To a solution of NaH (900.6 mg, 22.51 mmol) in THF (25 mL) under N2 at 0° C. was added ethane-1,2-diol (CXCV) (838.5 mg, 13.51 mmol) in THF (10 mL), and stirred at 30° C. for 0.5 h. Then 2,4-dichloro-5-iodo-7-((2-(trimethylsilyl)ethoxy)methyl)-7H-pyrrolo [2,3-d]pyrimidine (CXCIV) (commercially available from Advanced ChemBlocks Inc.) (5 g, 11.26 mmol) in THF (20 mL) was slowly added into the reaction solution at 0° C. The mixture was then stirred at 60° C. for 2 h. The reaction mixture was quenched with ice cold saturated aqueous NH₄C1 and diluted with EtOAc (240 mL) and washed with water (180 mL). The organic layer was filtered and concentrated. The residue was purified by silica gel column chromatography (20% EtOAc/hexanes) to afford 2-42-chloro-5-iodo-7-42-(trimethylsilypethoxy)methyl)-7H-pyrrolo[2,3-d]pyrimidin-4-ypoxy)ethan-1-ol (CXCVI) (3.174 g, 6.76 mmol, 60.0% yield) as a white solid. ESIMS found for C₁₄H₂₁ClIN₃O₃Si m/z 469.8 (M+H).

### Step 2

To a solution of 2-((2-chloro-5-iodo-7-((2-(trimethylsilyl)ethoxy)methyl)-7H-pyrrolo [2,3-d]pyrimidin-4-yl)oxy)ethan-1-ol (CXCVI) (1 g, 2.13 mmol), 644,4,5 ,5-tetramethyl-1,3,2-dioxaborolan-2-yl)imidazo [1,2-a]pyridine (CXCVII) (311.8 mg, 1.28 mmol) and K2CO₃ (882.7 mg, 6.39 mmol) in dioxane (20 mL) and H₂O (5 mL), Pd(dppf)Cl₂ (173.6 mg, 0.222 mmol) was added at room temperature under N2. The mixture was stirred at 120° C. for 3 h. The reaction mixture was filtered and diluted with H₂O (40 mL) and extracted with EtOAc (100 mL). The combined organic layers were concentrated to give the residue. The crude residue was purified by silica gel column chromatography (5% MeOH/DCM) to afford 2-42-chloro-5-(imidazo[1,2-a] pyridin-6-yl)-7-42-(trimethyl silypethoxy)methyl)-7H-pyrrolo [2,3-d]pyrimidin-4-yl)oxy)ethan-1-ol (CXCVIII) (646 mg, 1.40 mmol, 109%) as a brown solid. ESIMS found for C₂₁1-1₂₆ClN₅O₃Si m/z 460.0 (M+H).

### Step 3

To a solution of 2-((2-chloro-5-(imidazo[1,2-a]pyridin-6-yl)-7-42-(trimethylsilypethoxy)methyl)-7H-pyrrolo [2,3-d]pyrimidin-4-yl)oxy)ethan-1-ol (CXCVIII) (200 mg, 0.435 mmol) in DMF (6 mL) at room temperature was added imidazole (148.1 mg, 2.18 mmol). Then tert-butylchlorodiphenylsilane (CXCIX) (358.7 mg, 1.31 mmol) was added into the reaction solution slowly at 0° C. The mixture was stirred at room temperature for 0.5 h. The reaction mixture was diluted with EtOAc (240 mL) and washed with water (180 mL). The organic layer was concentrated. The residue was purified by silica gel column chromatography (12% MeOH/CHCl₃) to produce 4-(2-((teat-butyldiphenylsilyl) oxy)ethoxy)-2-chloro-5-(imidazo[1,2-a]pyridin-6-yl)-7-42-(trimethylsilypethoxy)methyl)-7H-pyrrolo [2,3-d]pyrimidine (CC) (308 mg, 0.441 mmol, 101% yield) as a brown oil. ESIMS found for C37H₄₄ClN503Si2m/z 698.1 (M+H).

### Step 4

To a solution of 4-(2-((tert-butyldiphenylsily0oxy)ethoxy)-2-chloro-5-(imidazo [1,2-a] pyridin-6-yl)-7-42-(trimethylsilypethoxy)methyl)-7H-pyrrolo [2,3-d] pyrimidine (CC) (278 mg, 0.398 mmol) in toluene (15 mL) was added t-BuONa (390.0 mg, 3. 98 mmol). The mixture was stirred at room temperature for 0.5 h. (R)-1,1,1-trifluoropropan-2-amine hydrochloride (CCI) (476.1 mg, 3.184 mmol), Pd₂(dba)₃ (72.9 mg, 0.0796 mmol) and BrettPhos (85.5 mg, 0.1592 mmol) was then added at room temperature. The mixture was stirred at 100° C. under N2 for 2 h. The mixture was filtered and diluted with DCM (180 mL) and washed with H₂O (80 mL). The organic layer was concentrated. The crude residue was purified by silica gel column chromatography (6% MeOH/CHCl₃) to give (R)-4-(2-((tert-butyldiphenylsilyl)oxy)ethoxy)-5-(imidazo [1,2-a] pyridin-6-yl)-N-(1,1, 1-trifluoropropan-2-yl)-7-42-(trimethyl silyl) ethoxy)methyl)-7H-pyrrolo [2,3-d]pyrimidin-2-amine (CCII) (262 mg, 0.338 mmol, 84.9% yield) as a brown oil. ESIMS found for C401149F3N603Si2m/z 775.1 (M+H).

### Step 5

To a solution of (R)-4-(2-((tert-butyldiphenylsilyl)oxy)ethoxy)-5-(imidazo [1,2-a] pyridin-6-yl)-N-(1,1, 1-trifluoropropan-2-yl)-7-42-(trimethyl silyl) ethoxy)methyl)-7H-pyrrolo [2,3-d]pyrimidin-2-amine (CCII) (175 mg, 0.226 mmol) was added TFA (1 mL) in DCM (3 mL). The mixture was stirred at room temperature for 16 h. The reaction mixture was were concentrated to give (R)-(4-(2-((tert-bu tyldiphenylsilyl)oxy)e thoxy)-5-(imidazo [1,2-a]pyridin-6-yl)-2-((1,1, 1-trifluoropropan-2-yl)amino)-7H-pyrrolo [2,3-d] pyrimidin-7-yl)methanol (CCIII) (132 mg, 0.196 mmol, 86.7% yield). The crude product was used directly for the next step without additional purification.

### Step 6

To a solution of (R)-(4-(2-((tert-butyldiphenylsilypoxy)ethoxy)-5-(imidazo[1,2-a]pyridin-6-yl)-2-((1,1,1-trifluoropropan-2-yl)amino)-7H-pyrrolo[2,3-d]pyrimidin-7-yl)methanol (CCIII) (132 mg, 0.196 mmol) in dioxane-HC1 (4M, 2 mL). The mixture was stirred at room temperature for 16 h. The combined organic layers were concentrated by co-evaporated with ether (3X) in a rotary evaporator to afford (R)-2-47-(hydroxymethyl)-5-(imidazo[1,2-a] pyridin-6-yl)-2-((1, 1,1-trifluoropropan-2-yl)amino)-7H-pyrrolo [2,3-d]pyrimidin-4-yl)oxy)ethan-1-ol (CCIV) (92 mg, 0.211 mol, 107% yield). The crude product was used directly for the next step without additional purification.

### Step 7

To a solution of (R)-2-((7-(hydroxymethyl)-5-(imidazo[1,2-a]pyridin-6-yl)-2-((1, 1,1-trifluoropropan-2-yl)amino)-7H-pyrrolo [2,3-d] pyrimidin-4-yl)oxy)ethan-1-ol (CCIV) (92 mg, 0.211 mol), K₂CO₃ (145.6 mg, 1.055 mol) in MeOH/H₂O (5 mL/1 mL). The mixture was stirred at room temperature for 1.5 h. After completion, the mixture was diluted with EtOAc and washed with H₂O (120 mL). The organic layer was concentrated. The crude residue were purified by Prep-HPLC to yield (R)-2-((5-(Imidazo[1,2-a]pyridin-6-yl)-2-((1,1,1-trifluoropropan-2-yl)amino)-7H-pyrrolo [2,3-d]pyrimidin-4-yl)oxy)ethan-1-ol (2178) (26.14 mg, 0.064 mmol, 30.3% yield) as a white solid. ¹H NMR (400 MHz, DMSO-d₆) δppm 1.35 (3 H, d, J=7.00 Hz), 3.81 (2 H, t, J=4.82 Hz), 4.44-4.52 (2 H, m), 4.83-4.97 (1 H, m), 5.05 (1 H, br s), 7.24 (1 H, d, J=9.13 Hz), 7.37 (1 H, d, J=2.38 Hz), 7.49-7.52 (1 H, m), 7.53 (1 H, s), 7.61 (1 H, dd, J=9.51, 1.63 Hz), 7.88 (1 H, s), 9.12 (1 H, s), 11.59 (1 H, br s); ESIMS found for C₁₈H₁₇F₃N₆O₂ m/z 407.4 (M+1).

### Example 11

Preparation of N-((lR,5 S,6 s)-3-oxabicyclo [3 .1 . 0] hexan-6-yl)-4-methoxy-5-(pyrazolo [1,5-a] pyridin-5-yl)-7H-pyrrolo [2,3-d]pyrimidin-2-amine (2324) and 4-methoxy-5-(pyrazolo [1,5-a] pyridin-5-yl)-7H-pyrrolo [2,3-d] pyrimidin-2-amine (3291) are depicted below in Scheme 39.

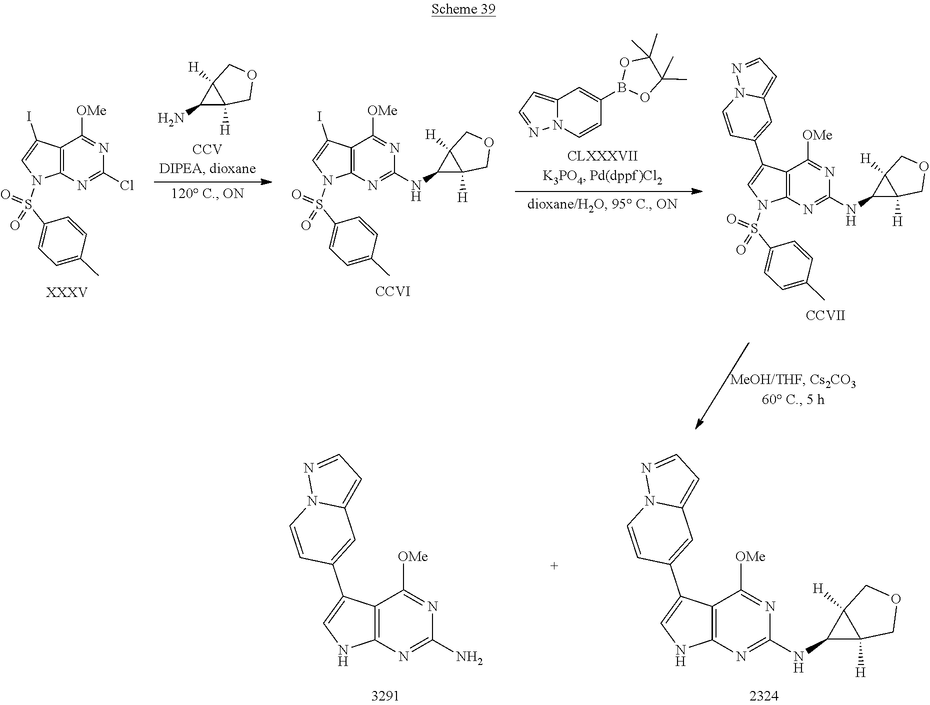

### Step 1

To a stirred solution of 2-chloro-5-iodo-4-methoxy-7-tosyl-7H-pyrrolo[2,3-d]pyrimidine (XXXV) (400 mg, 0.86 mmol) in 1,4-dioxane (4 mL) were added (1R,5S,6s)-3-oxabicyclo[3.1.0]hexan-6-amine hydrochloride (CCV) (commercially available from WuXi LabNetwork) (140 mg, 1.03 mmol) and DIPEA (450 uL, 2.58 mmol). The reaction mixture stirred at 120° C. overnight . The reaction mixture was quenched with water, taken into EtOAc, washed with brine and dried over anhydrous Na₂SO₄, solvents concentrated under reduced pressure and the crude residue was purified by ISCO (0-400% EtOAc/hexanes). Fractions were collected and dried under high vacuo to obtain N-((lR,5S,6s)-3-oxabicyclo[3.1.0]hexan-6-yl)-5-iodo-4-methoxy-7-tosyl-7H-pyrrolo[2,3-d]pyrimidin-2-amine (CCVI) (184 mg, 0.350 mmol, 40.5% yield) as a brown solid. ESIMS found for C₁₉H₁₉IN404S m/z 527.1 (M+H).

### Step 2

To a stirred solution of N-((lR,5 S,6s)-3-oxabicyclo [3 .1. O]hexan-6-yl)-5-iodo-4-methoxy-7-tosyl-7H-pyrrolo [2,3-d]pyrimidin-2-amine (CCVI) (110 .mg, 0.21 mmol), 5-(4,4,5,5-tetramethyl-1,3,2-dioxaborolan-2-yl)pyrazolo[1,5-a]pyridine (CLXXXVII) (77 mg, 0.32 mmol) and Pd(dppf)C12 (11 mg, 0.010 mmol) in 1,4-dioxane (3 mL) was added a 2 M aqueous solution of K₃PO₄ (314 μL, 0.63 mmol). The reaction mixture was purged with N₂ gas for 5 min, the vial was sealed and heated at 95° C. overnight. The organic layer was separated, absorbed on silica, and purified by ISCO (0→80% EtOAc/hexanes). Fractions were collected and dried under high vacuo to obtain N-((lR,5 S,6s)-3-oxabicyclo [3 . 1. O]hexan-6-yl)-4-methoxy-5-(pyrazolo [1,5-a]pyridin-5-yl)-7-tosyl-7H-pyrrolo[2,3-d]pyrimidin-2-amine (CCVII) (92 mg, 0.178 mmol, 85.2% yield) as a beige solid. ESIMS found for C26H₂₄N604S m/z 517.2 (M+H).

### Step 3

To a stirred solution of N-((lR,5S,6s)-3-oxabicyclo[3.1.0]hexan-6-yl)-4-methoxy-5-(pyrazolo [1,5-a]pyridin-5-yl)-7-to syl-7H-pyrrolo [2,3-d]pyrimidin-2-amine (CCVII) (60 mg, 0.12 mmol) in MeOH (1.5 mL), THF (1.5 mL) and water (0.50 mL) was added Cs₂CO₃ (189 mg, 0.58 mmol) at room temperature. The reaction mixture heated at 60° C. for 5 h. The solvent was evaporated under vacuum, and the crude mixture was purified on ISCO (0→20% 7% NH₃ in MeOH/CHCl₃). Appropriate fractions were collected dried under high vacuo to obtain N-((1R,5 S,6 s)-3-oxabicyclo [3.1.0] hexan-6-yl)-4-methoxy-5-(pyrazolo [1,5-a]pyridin-5-yl)-7H-pyrrolo[2,3-d]pyrimidin-2-amine (2324) (12 mg, 0.033 mmol, 28.5% yield) as a beige solid. ¹H NMR (499 MHz, DMSO-d₆) δppm 1.83 (1 H, s), 1.99 (1 H, br d, J=6.84 Hz), 2.51-2.56 (1 H, m), 3.66 (1 H, br d, J=7.94 Hz), 3.84 (2 H, s), 3.88 (1 H, d, J=8.21 Hz), 3.99 (3 H, s), 6.23 (1 H, d, J=3.56 Hz), 6.55 (1 H, s), 7.19 (1 H, dd, J=7.39, 1.92 Hz), 7.37 (1 H, t, J=2.33 Hz), 7.93 (1 H, d, J=2.19 Hz), 7.99 (1 H, s), 8.58 (1 H, d, J=7.39 Hz), 11.58 (1 H, br s); ESIMS found for C₁₉H₁₈N₆O₂ m/z 363.2 (M+1) and 4-methoxy-5-(pyrazolo [1,5-a]pyridin-5-yl)-7H-pyrrolo [2,3-d]pyrimidin-2-amine (3291) (10 mg, 0.036 mmol, 30.7% yield) as a beige solid. ¹H NMR (499 MHz, DMSO-d₆) δppm 3.96 (3 H, s), 6.17 (2 H, s), 6.54 (1 H, d, J=2.19 Hz), 7.18 (1 H, dd, J=7.39, 1.92 Hz), 7.35 (1 H, d, J=2.46 Hz), 7.93 (1 H, d, J=2.19 Hz), 7.99 (1 H, d, J=1.09 Hz), 8.58 (1 H, d, J=7.39 Hz), 11.43 (1 H, br s); ESIMS found for C₁₄H₁₂N₆O m/z 281.2 (M+1).

### Example 12

Preparation of (R)-5-(3-i sobutyl-2-methyl-3H-imidazo [4,5-b]pyridin-5-yl)-2-methoxy-N-(1, 1,1-trifluoropropan-2-yl)-7H-pyrrolo [2,3-d]pyrimidin-4-amine (2817) is depicted below in Scheme 40.

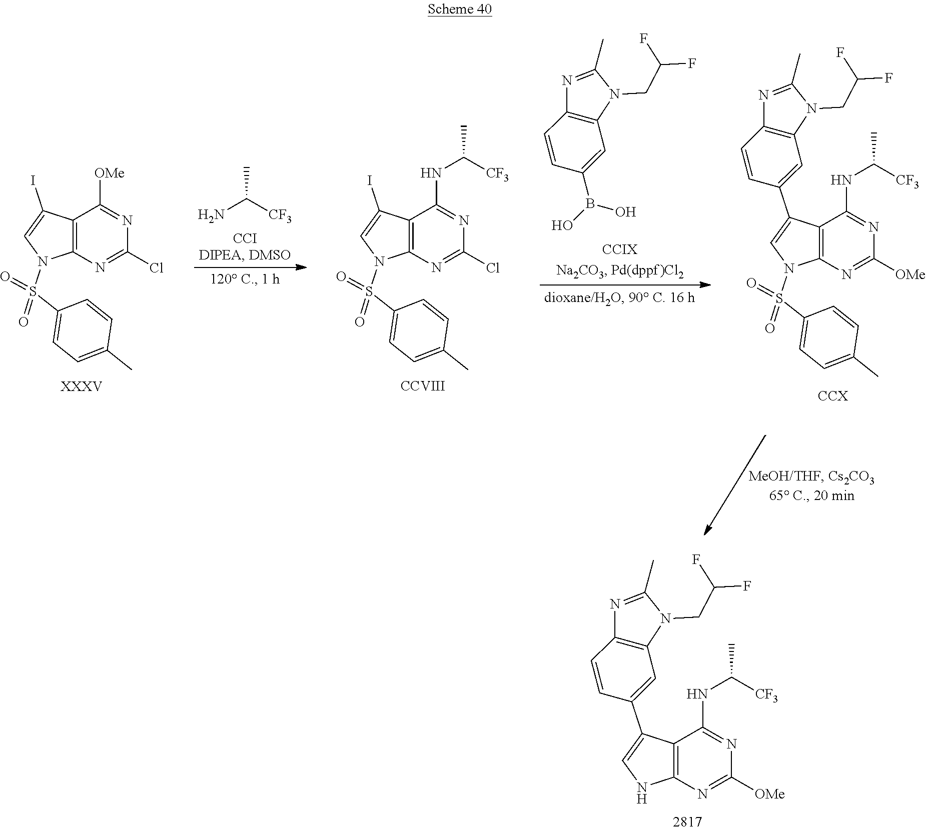

### Step 1

To a solution of 2-chloro-5-iodo-4-methoxy-7-tosyl-7H-pyrrolo[2,3-d]pyrimidine (XXXV) (500 mg, 1.08 mmol) and (R)-1,1,1-trifluoropropan-2-amine hydrochloride (CCI) (330 mg, 2.21 mmol) in DMSO (1.5 mL) was added DIPEA (808 uL, 4.64 mmol). The reaction was stirred at 120° C. for 1 h. The reaction mixture was added to EtOAc, and water and the organic layer was separated. The aqueous layer was extracted with EtOAc (×3), and the combined organic layers were washed with brine, dried (anhydrous MgSO₄), and reduced in vacuo to give the crude product as a brown semi-solid. This was purified by column chromatography (0→60% EtOAc/hexanes) to give the product as a brown semi-solid. This was triturated and sonicated in MTBE and hexanes and the solvent was evaporated to give (R)-5-iodo-2-methoxy-7-tosyl-N-(1,1,1-trifluoropropan-2-yl)-7H-pyrrolo [2,3-d]pyrimidin-4-amine (CCVIII) (109 mg, 0.202 mmol, 18.7% yield) as a brown solid. ESIMS found for C₁₇H₁₆F₃IN₄O₃S m/z 541.1 (M+H).

### Step 2

To a suspension of (R)-5-iodo-2-methoxy-7-tosyl-N-(1,1,1-trifluoropropan-2-yl)-7H-pyrrolo[2,3-d]pyrimidin-4-amine (CCVIII) (39 mg, 0.07 mmol), [3-(2,2-difluoroethyl)-2-methylimidazo[4,5-b]pyridin-5-yl]boronic acid (CCIX) (26 mg, 0.11 mmol) and Pd(dppf)C12 (3 mg, 0.004 mmol) in 1,4-dioxane (0.75 mL) was added a aqueous solution of Na2CO₃ (229 μL, 0.22 mmol). The reaction was sonicated and degassed for 5 minutes before heating at 90° C. for 16 h. The reaction mixture as purified by column chromatography (0→100% EtOAc/hexanes) to produce (R)-5-(3-(2,2-difluoroethyl)-2-methyl-3H-imidazo [4,5-b]pyridin-5-yl)-2-methoxy-7-to syl-N-(1,1,1-trifluoropropan-2-yl)-7H-pyrrolo [2,3-d]pyrimidin-4-amine (CCX) (33 mg, 0.054 mmol, 75.0% yield) as an off-white solid. ESIMS found for C₂₆H₂₄F₅N₇O₃S m/z 610.2 (M+H).

### Step 3

To a solution of (R)-5-(3-(2,2-difluoroethyl)-2-methyl-3H-imidazo[4,5-b]pyridin-5-yl)-2-methoxy-7-tosyl-N-(1, 1, 1-trifluoropropan-2-yl)-7H-pyrrolo [2,3-d]pyrimidin-4-amine (CCX) (33 mg, 0.05 mmol) in THF (1 mL) and MeOH (1 mL) was added Cs₂CO₃ (53 mg, 0.16 mmol). The reaction was heated to 65° C. for 20 min. The reaction was cooled to room temperature and purified directly by reverse phase column chromatography (10->35% MeCN/H₂O in 0.1% formic acid). Appropriate fractions were collected and added to saturated aqueous NaHCO₃ and extracted with DCM (x2). The combined organic layers were dried (anhydrous MgSO₄) and reduced in vacuo to obtain (R)-5-(3-isobutyl-2-methyl-3H-imidazo[4,5-b]pyridin-5-yl)-2-methoxy-N-(1,1,1-trifluoropropan-2-yl)-7H-pyrrolo [2,3-d]pyrimidin-4-amine (2817) (16 mg, 0.035 mmol, 64.9% yield) as an off-white solid. ¹H NMR (499 MHz, DMSO-d₆) δppm 1.45 (3 H, d, J=7.12 Hz), 2.58 (3 H, s), 3.45 (3 H, s), 4.73 (2 H, td, J=15.40, 3.42 Hz), 5.07 (1 H, dq, J=15.37, 7.75 Hz), 6.53 (2 H, tt, J=55.10, 3.60 Hz), 7.05 (1 H, d, J=8.49 Hz), 7.48 (1 H, d, J=2.46 Hz), 7.86 (1 H, d, J=8.21 Hz), 8.62 (1 H, d, J=8.21 Hz), 11.59 (1 H, s); ESIMS found for C₁₉H₁₈F₅N₇O m/z 456.2 (M+1).

### Example 13

Preparation of trans-1-methyl-4-((4-(methylamino)-5-(pyrazolo [1,5-a]pyrimidin-5-yl)-7H-pyrrolo [2,3-d]pyrimidin-2-yl)amino)cyclohexan-1-ol (3454) is depicted below in Scheme 41.

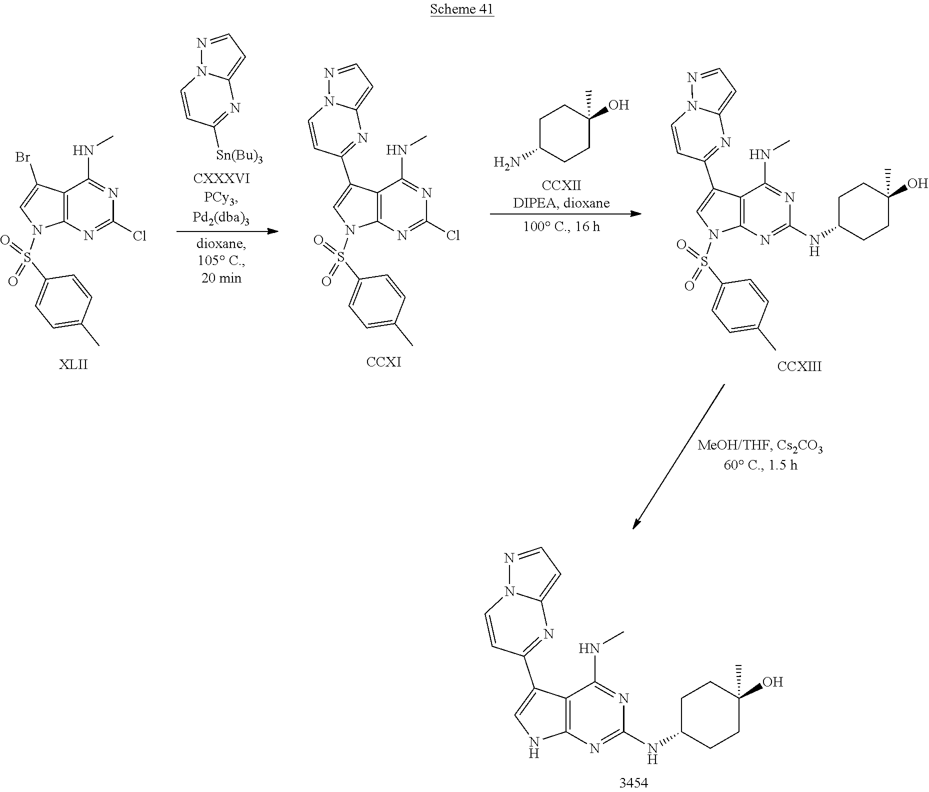

### Step 1

To a solution of 5-bromo-2-chloro-N-methyl-7-tosyl-7H-pyrrolo[2,3-d]pyrimidin-4-amine (XLII) (200 mg, 0.48 mmol), 5-(tributylstannyl)pyrazolo[1,5-a]pyrimidine (CXXXVI) (255 mg, 0.62 mmol), Pd₂(dba)₃ (44 mg, 0.05 mmol) and PCy₃ (27 mg, 0.1 mmol) in dry 1,4-dioxane (3 mL) in a microwave vial were purged with Ar for 5 min. The reaction mixture was stirred at 105° C. for 20 min. The reaction mixture was added to Celite® and purified by column chromatography (isocratic: CHCl₃). Appropriate fractions were collected and reduced in vacuo to give a yellow solid. The solid was triturated with MeOH and filtered to give the product 2-chloro-N-methyl-5-(pyrazolo [1,5-a] pyrimidin-5-yl)-7-to syl-7H-pyrrolo [2,3-d] pyrimidin-4-amine (CCXI) (130 mg, 0.286 mmol, 59.5% yield) as a white solid. ESIMS found for C₂₀H₁₆ClN702S m/z 454.1 (M+H).

### Step 2

To a suspension of 2-chloro-N-methyl-5-(pyrazolo[1,5-a]pyrimidin-5-yl)-7-tosyl-7H-pyrrolo[2,3-d]pyrimidin-4-amine (CCXI) (50 mg, 0.11 mmol) and trans-4-amino-1-methylcyclohexan-1-ol (CCXII) (commercially available from PharmaBlock (USA), Inc.) (18 mg, 0.14 mmol) in 1,4-dioxane (150 μL) was added DIPEA (39 uL, 0.22 mmol). The reaction was heated to 100° C. for 16 h. Crude reaction mixture was loaded onto Celite® and purified by column chromatography (0→80% EtOAc/hexanes). The product trans-1-methyl-4-44-(methylamino)-5-(pyrazolo [1,5-a] pyrimidin-5-yl)-7-to syl-7H-pyrrolo [2,3-d] pyrimidin-2-yl)amino)cyclohexan-1-ol (CCXIII) (5 mg, 0.009 mmol, 8.3% yield) as an off-white solid. ESIMS found for C27H₃₀N803S m/z 547.3 (M+H).

### Step 3

To a suspension of trans-1-methyl-4-((4-(methylamino)-5-(pyrazolo[1,5-a] pyrimidin-5-yl)-7-to syl-7H-pyrrolo [2,3-d] pyrimidin-2-yl)amino)cyclohexan-1-ol (CCXIII) (5 mg, 0.01 mmol) in MeOH (1 mL) and THF (1 mL) was added Cs₂CO₃ (6 mg, 0.02 mmol). The reaction was heated 60° C. for 1.5 h. The reaction mixture was directly purified by HPLC (0->35% MeCN/H₂O in 0.1% formic acid). Appropriate fractions were collected and neutralized with saturated aqueous NaHCO₃ and extracted with DCM (x2). The combined organic layers were dried (anhydrous MgSO₄) and reduced in vacuo to obtain trans-1-methyl-4-44-(methylamino)-5-(pyrazolo [1,5-a] pyrimidin-5-yl)-7H-pyrrolo [2,3-d]pyrimidin-2-yl)amino)cyclohexan-1-ol (3454) (2.5 mg, 0.006 mmol, 69.6% yield) as a yellow solid. ¹H NMR (499 MHz, DMSO-d₆) δppm 1.14 (3 H, s), 1.42 (4 H, br d, J=9.03 Hz), 1.55-1.61 (2 H, m), 1.80-1.88 (2 H, m), 3.03 (3 H, d, J=4.65 Hz), 3.70-3.83 (1 H, m), 4.20 (1 H, s), 5.93 (1 H, d, J=8.21 Hz), 6.62 (1 H, dd, J=2.19, 0.82 Hz), 7.48 (1 H, d, J=7.67 Hz), 7.91 (1 H, d, J=2.74 Hz), 8.09 (1 H, d, J=2.46 Hz), 8.90 (1 H, d, J=7.39 Hz), 9.93 (1 H, q, J=4.56 Hz), 11.62 (1 H, br s); ESIMS found for C₂₀I-1₂₄N₈O m/z 393.25 (M+1).

### Example 14

Preparation of (2-(4-(methylamino)-5-(pyrazolo [1,5-a] pyrimidin-5-yl)-7H-pyrrolo[2,3-d]pyrimidin-2-yl)-2-azabicyclo[2.2.2]octan-4-yl)methanol (3459) is depicted below in Scheme 42.

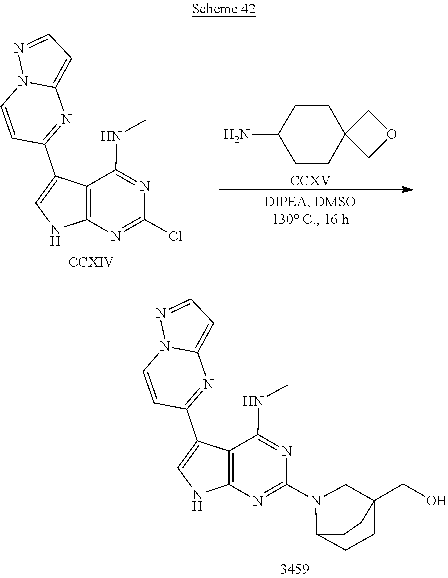

### Step 1

To a solution of 2-chloro-N-methyl-5-(pyrazolo[1,5-a]pyrimidin-5-yl)-7H-pyrrolo[2,3-d]pyrimidin-4-amine (CCXIV) (18 mg, 0.06 mmol) and 2-oxaspiro[3.5]nonan-7-amine (CCXV) (commercially available from PharmaBlock (USA), Inc.) (13 mg, 0.09 mmol) in DMSO (0.1000 mL) was added DIPEA (31 μL, 0.18 mmol). The reaction was heated to 130° C. for 16 h. The reaction mixture was purified directly by HPLC (0->40% MeCN/H₂₀ in 0.1% formic acid). Appropriate fractions for both peaks were collected and neutralized with saturated aqueous NaHCO₃ and extracted with DCM (x2). The combined organic layers for each product were dried (anhydrous MgSO₄) and reduced in vacuo to give a yellow semi solid. The major product was further purified by column chromatography (0->5% 7.0 M NH₃ in MeOH/CHCl₃) to obtain (2-(4-(methylamino)-5-(pyrazolo [1,5-a] pyrimidin-5-yl)-7H-pyrrolo [2,3-d] pyrimidin-2-yl)-2-azabicyclo[2.2.2]octan-4-yl)methanol (3459) (5 mg, 0.012 mmol, 20.6% yield) as a yellow solid. ¹H NMR (499 MHz, DMSO-d₆) δppm 1.37-1.53 (4 H, m), 1.68 (2 H, br s), 1.77 (2 H, br d, J=3.29 Hz), 3.05 (3 H, br s), 3.21 (2 H, d, J=5.20 Hz), 3.33-3.45 (2 H, m), 4.55 (1 H, t, J=5.34 Hz), 4.58-4.90 (1 H, m), 6.62 (1 H, d, J=2.19 Hz), 7.49 (1 H, d, J=7.39 Hz), 7.91 (1 H, d, J=3.01 Hz), 8.09 (1 H, d, J=2.19 Hz), 8.90 (1 H, d, J=7.67 Hz), 9.88-9.99 (1 H, m), 11.57 (1 H, br s); ESIMS found for C2,H₂₄N80 m/z 405.3 (M+1).

The following compounds were prepared in accordance with the procedures described in the above Schemes 1-42.

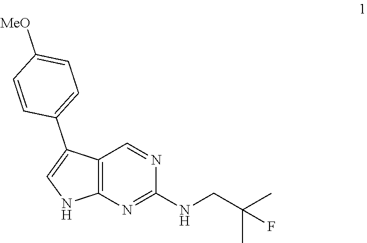 [SMILES: COc1ccc(-c2c[nH]c3nc(NCC(C)(C)F)ncc23)cc1]

N-(2-Fluoro-2-methylpropyl)-5-(4-methoxyphenyl)-7H-pyrrolo [2,3-d] pyrimidin-2-amine 1.

White solid (18 mg, 0.057 mmol, 32.9% yield). ¹H NMR (499 MHz, DMSO-d₆) δppm 1.34 (6 H, d, J=21.40 Hz), 3.58 (2 H, dd, J=18.89, 6.30 Hz), 3.78 (3 H, s), 6.78 (1 H, br t, J=6.30 Hz), 6.93-7.01 (2 H, m), 7.31 (1 H, d, J=1.64 Hz), 7.55-7.63 (2 H, m), 8.80 (1 H, s), 11.35 (1 H, br s); ESIMS found for C₁₇H₁₉FN₄O m/z 315.15 (M+1).

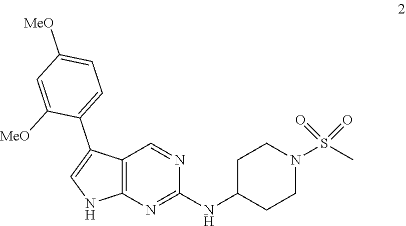 [SMILES: COc1ccc(-c2c[nH]c3nc(NC4CCN(S(C)(=O)=O)CC4)ncc23)c(OC)c1]

5-(2,4-Dimethoxyphenyl)-N-(1-(methyl sulfonyl)piperidin-4-yl)-7H-pyrrolo [2,3-d]pyrimidin-2-amine 2.

White solid (33.9 mg, 0.079 mmol, 62.1% yield). ¹HNMR (499 MHz, DMSO-d₆) δppm 1.52-1.65 (2 H, m), 2.00 (2 H, br dd, J=13.14, 3.29 Hz), 2.84-2.91 (2 H, m), 2.88 (3 H, s), 3.51-3.59 (2 H, m), 3.79 (3 H, s), 3.81 (3 H, s), 3.84-3.93 (1 H, m), 6.59 (1 H, dd, J=8.49, 2.46 Hz), 6.66 (1 H, d, J=2.74 Hz), 6.67 (1 H, br d, J=8.21 Hz), 7.16 (1 H, d, J=2.19 Hz), 7.42 (1 H, d, J=8.76 Hz), 8.57 (1 H, s), 11.29 (1 H, br s); ESIMS found for C₂₀I-1₂₅N₅O₄S m/z 432.2 (M+1).

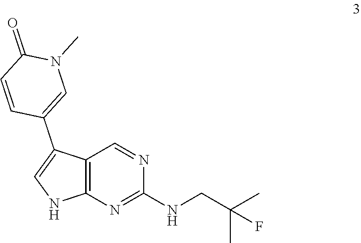 [SMILES: Cn1cc(-c2c[nH]c3nc(NCC(C)(C)F)ncc23)ccc1=O]

5-(2-((2-Fluoro-2-methylpropyl)amino)-7H-pyrrolo [2,3-d]pyrimidin-5-yl)-1-methylpyridin-2(1H)-one 3.

White solid (6 mg, 0.019 mmol, 24.8% yield). ¹H NMR (499 MHz, DMSO-d₆) δppm 1.34 (6 H, d, J=21.40 Hz), 3.52 (3 H, s), 3.57 (2 H, dd, J=19.16, 6.57 Hz), 6.46 (1 H, d, J=9.31 Hz), 6.82 (1 H, br t, J=6.30 Hz), 7.31 (1 H, d, J=2.19 Hz), 7.80 (1 H, dd, J=9.58, 2.46 Hz), 7.98 (1 H, d, J=2.19 Hz), 8.88 (1 H, s), 11.36 (1 H, br s); ESIMS found for C₁₆HisFN₅O m/z 316.1 (M+1).

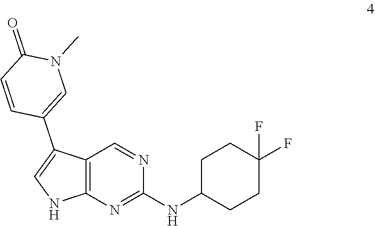 [SMILES: Cn1cc(-c2c[nH]c3nc(NC4CCC(F)(F)CC4)ncc23)ccc1=O]

5-(2-((4,4-Difluorocyclohexyl)amino)-7H-pyrrolo [2,3-d]pyrimidin-5-yl)-1-methylpyridin-2(1H)-one 4.

Beige solid (40 mg, 0.111 mmol, 22.7% yield). ¹H NMR (499 MHz, DMSO-d₆) δppm 1.54-1.67(2 H, m), 1.84-2.00 (4 H, m), 2.04-2.13 (2 H, m), 3.52(3 H, s), 3.86-3.98 (1 H, m), 6.46 (1 H, d, J=9.31 Hz), 6.81 (1 H, br d, J=7.67 Hz), 7.30 (1 H, d, J=2.19 Hz), 7.80 (1 H, dd, J=9.31, 2.74 Hz), 7.97 (1 H, d, J=2.19 Hz), 8.88 (1 H, s), 11.37 (1 H, br s); ESIMS found for C₁₈H₁₉F₂N₅O m/z 360.2 (M+1).

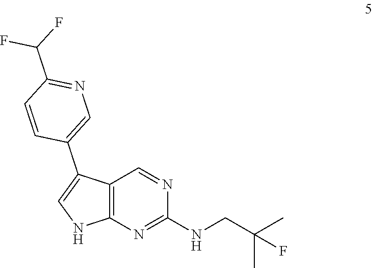 [SMILES: CC(C)(F)CNc1ncc2c(-c3ccc(C(F)F)nc3)c[nH]c2n1]

5-(6-(Difluoromethyl)pyridin-3-yl)-N-(2-fluoro-2-methylpropyl)-7H-pyrrolo [2,3-d]pyrimidin-2-amine 5.

White solid (18 mg, 0.054 mmol, 20.6% yield). ¹H NMR (499 MHz, DMSO-d₆) δppm 1.35 (6 H, d, J=21.40 Hz), 3.59 (2 H, dd, J=19.16, 6.57 Hz), 6.96 (1 H, t, J=54.90 Hz), 6.94 (1 H, br s), 7.69 (1 H, d, J=8.21 Hz), 7.75 (1 H, s), 8.28 (1 H, dd, J=8.21, 2.19 Hz), 8.96 (1 H, s), 9.04 (1 H, d, J=2.19 Hz), 11.74 (1 H, br s); ESIMS found for C₁₆H₁₆F₃N₅ m/z 336.15 (M+1).

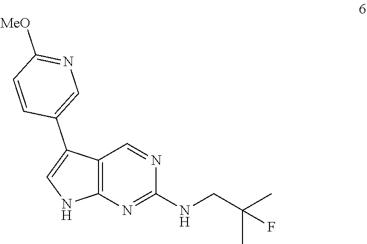 [SMILES: COc1ccc(-c2c[nH]c3nc(NCC(C)(C)F)ncc23)cn1]

N-(2-Fluoro-2-methylpropyl)-5-(6-methoxypyridin-3-yl)-7H-pyrrolo [2,3-d] pyrimidin-2-amine 6.

White solid (18 mg, 0.057 mmol, 25.7% yield). ¹H NMR (499 MHz, DMSO-d₆) δppm 1.34 (6 H, d, J=21.40 Hz), 3.58 (2 H, dd, J=19.16, 6.02 Hz), 3.88 (3 H, s), 6.82 (1 H, br t, J=6.02 Hz), 6.86 (1 H, d, J=8.21 Hz), 7.43 (1 H, s), 8.02 (1 H, dd, J=8.49, 2.46 Hz), 8.50 (1 H, d, J=2.19 Hz), 8.83 (1 H, s), 11.47 (1 H, br s); ESIMS found for C₁₆H₁₈FN₅O m/z 316.2 (M+1).

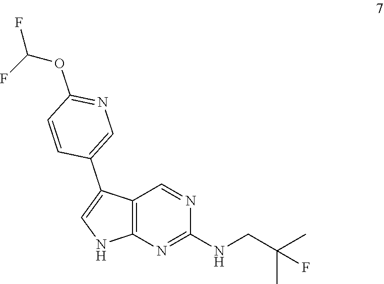 [SMILES: CC(C)(F)CNc1ncc2c(-c3ccc(OC(F)F)nc3)c[nH]c2n1]

5-(6-(Difluoromethoxy)pyridin-3-yl)-N-(2-fluoro-2-methylpropyl)-7H-pyrrolo[2,3-d]pyrimidin-2-amine 7.

Light brown solid (50 mg, 0.142 mmol, 64.2% yield). ¹1-1 NMR (499 MHz, DMSO-d₆) δppm 1.34 (6 H, d, J=21.40 Hz), 3.58 (2 H, dd, J=18.89, 6.30 Hz), 6.82-6.92 (1 H, m), 7.12 (1 H, d, J=8.76 Hz), 7.57 (1 H, s), 7.71 (1 H, t, J=73.00 Hz), 8.19-8.26 (1 H, m), 8.60 (1 H, d, J=2.19 Hz), 8.88 (1 H, s), 11.60 (1 H, br s); ESIMS found for C₁₆K₆F₃N₅O m/z 352.1 (M+1).

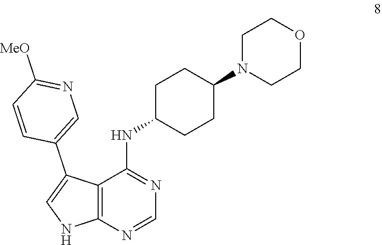 [SMILES: COc1ccc(-c2c[nH]c3ncnc(N[C@H]4CC[C@H](N5CCOCC5)CC4)c23)cn1]

5-(6-Methoxypyridin-3-yl)-N-(trans-4-morpholinocyclohexyl)-7H-pyrrolo [2,3-d]pyrimidin-4-amine 8.

Off-white solid (5 mg, 0.012 mmol, 9.6% yield). ¹H NMR (499 MHz, DMSO-d₆) δppm 1.07-1.19 (2 H, m), 1.25-1.37 (2 H, m), 1.80 (2 H, br d, J=11.80 Hz), 2.03 (2 H, br d, J=9.88 Hz), 2.11-2.20 (1 H, m), 2.42-2.47 (4 H, m), 3.51-3.58 (4 H, m), 3.90 (3 H, s), 3.91-4.00 (1 H, m), 5.01 (1 H, d, J=7.68 Hz), 6.92 (1 H, d, J=8.51 Hz), 7.22 (1 H, d, J=1.65 Hz), 7.77 (1 H, dd, J=8.51, 2.47 Hz), 8.17 (1 H, s), 8.25 (1 H, d, J=1.92 Hz), 11.83 (1 H, s); ESIMS found for C22H₂₈N602 m/z 409.2 (M+1).

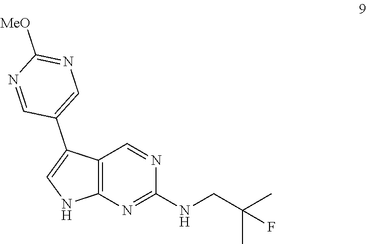 [SMILES: COc1ncc(-c2c[nH]c3nc(NCC(C)(C)F)ncc23)cn1]

N-(2-Fluoro-2-methylpropyl)-5-(2-methoxypyrimidin-5-yl)-7H-pyrrolo [2,3-d]pyrimidin-2-amine 9.

White solid (10 mg, 0.032 mmol, 16.6% yield). ¹H NMR (499 MHz, DMSO-d₆) δppm 1.34 (6 H, d, J=21.40 Hz), 3.58 (2 H, dd, J=19.16, 6.57 Hz), 3.95 (3 H, s), 6.87 (1 H, t, J=5.20 Hz), 7.57 (1 H, s), 8.88 (1 H, s), 8.94 (2 H, s), 11.60 (1 H, br s); ESIMS found for C₁₅H₁₇FN₆O m/z 317.1 (M+1).

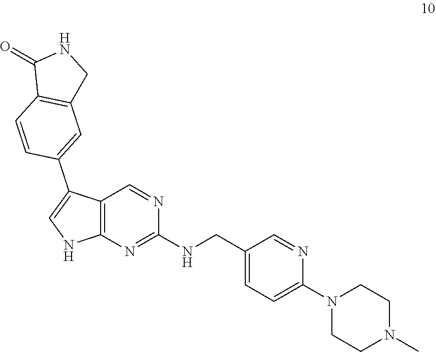 [SMILES: CN1CCN(c2ccc(CNc3ncc4c(-c5ccc6c(c5)CNC6=O)c[nH]c4n3)cn2)CC1]

5-(2-(((6-(4-Methylpiperazin-1-yl)pyridin-3-yl)methyl)amino)-7H-pyrrolo [2,3-d]pyrimidin-5-yl)i soindolin-1-one 10.

Off-white solid (9 mg, 0.020 mmol, 46.4% yield). ¹H NMR (499 MHz, DMSO-d₆) δppm 2.19 (3 H, s), 2.33-2.39 (4 H, m), 3.37-3.44 (4 H, m), 4.39 (2 H, d, J=6.30 Hz), 4.40 (2 H, s), 6.76 (1 H, d, J=8.76 Hz), 7.28 (1 H, br t, J=5.48 Hz), 7.54 (1 H, dd, J=8.76, 2.46 Hz), 7.59 (1 H, s), 7.65 (1 H, d, J=7.94 Hz), 7.79 (1 H, dd, J=8.08, 1.23 Hz), 7.88 (1 H, s), 8.12 (1 H, d, J=2.19 Hz), 8.43 (1 H, s), 8.96 (1 H, s), 11.60 (1 H, br s); ESIMS found for C₂₅H₂₆N₈O m/z 455.1 (M+1).

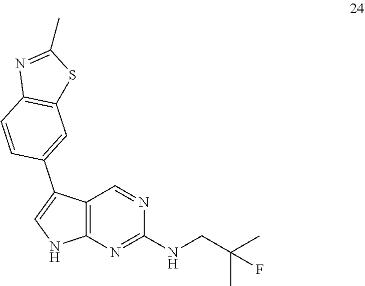 [SMILES: Cc1nc2ccc(-c3c[nH]c4nc(NCC(C)(C)F)ncc34)cc2s1]

N-(2-Fluoro-2-methylpropyl)-5-(2-methylbenzo[d]thiazol-6-yl)-7H-pyrrolo [2,3-d]pyrimidin-2-amine 24.

White solid (18.3 mg, 0.051 mmol). ¹H NMR (400 MHz, DMSO-d₆) δppm 1.35 (6 H, d, J=21.40 Hz), 2.79 (3 H, s), 3.59 (2 H, dd, J=18.89, 6.30 Hz), 6.86 (1 H, br t, J=6.11 Hz), 7.54 (1 H, d, J=2.32 Hz), 7.80 (1 H, dd, J=8.44, 1.71 Hz), 7.87-7.94 (1 H, m), 8.35 (1 H, d, J=1.47 Hz), 8.99 (1 H, s), 11.52 (1 H, br s); ESIMS found for C₁₈H₁₈FN₅S m/z 356.1 (M+1).

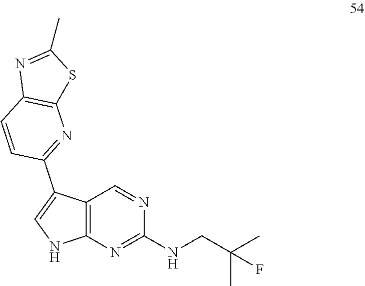 [SMILES: Cc1nc2ccc(-c3c[nH]c4nc(NCC(C)(C)F)ncc34)nc2s1]

N-(2-Fluoro-2-methylpropyl)-5-(2-methylthiazolo [5,4-b] pyridin-5-yl)-7H-pyrrolo [2,3-d] pyrimidin-2-amine 54.

White solid (8.53 mg, 0.024 mmol). ¹H NMR (400 MHz, DMSO-d₆) δppm 1.35 (6 H, d, J=21.40 Hz), 2.82 (3 H, s), 3.59 (2 H, dd, J=18.95, 6.44 Hz), 6.92 (1 H, br t, J=6.13 Hz), 7.94 (1 H, d, J=2.50 Hz), 7.98 (1 H, d, J=8.63 Hz), 8.21 (1 H, d, J=8.63 Hz), 9.27 (1 H, s), 11.69 (1 H, br s); ESIMS found for C₁₇H₁₇FN₆S m/z 357.2 (M+1).

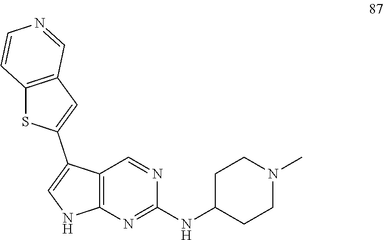 [SMILES: CN1CCC(Nc2ncc3c(-c4cc5cnccc5s4)c[nH]c3n2)CC1]

N-(1-Methylpiperidin-4-yl)-5-(thieno[3,2-c]pyridin-2-yl)-7H-pyrrolo[2,3-d] pyrimidin-2-amine 87.

Brown solid (5.2 mg, 0.014 mmol, 24.7% yield). ¹H NMR (499 MHz, DMSO-d₆) δppm 1.53 (2 H, qd, J=11.73, 3.70 Hz), 1.87 (2 H, br d, J=10.13 Hz), 1.92-2.00 (2 H, m), 2.17 (3 H, s), 2.76(2 H, br d, J=11.77 Hz), 3.64-3.76 (1 H, m), 6.81 (1 H, br d, J=4.93 Hz), 7.58 (1 H, s), 7.82 (1 H, s), 7.98 (1 H, d, J=5.48 Hz), 8.35 (1 H, d, J=5.48 Hz), 8.99 (1 H, s), 9.01 (1 H, d, J=0.82 Hz), 11.76 (1 H, br s); ESIMS found for C₁₉H₂₀N₆S m/z 365.1 (M+1).

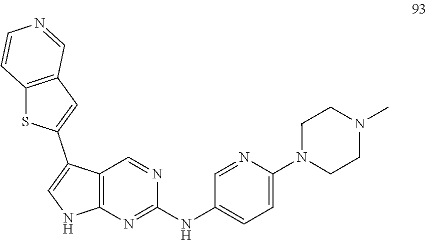 [SMILES: CN1CCN(c2ccc(Nc3ncc4c(-c5cc6cnccc6s5)c[nH]c4n3)cn2)CC1]

N-(6-(4-Methylpipe razin-1-yl)pyridin-3-yl)-5-(thieno [3,2-c]pyridin-2-yl)-7H-pyrrolo[2,3-d]pyrimidin-2-amine 93.

Off-white solid (1.8 mg, 0.004 mmol, 30.3% yield). ¹H NMR (499 MHz, DMSO-d₆) δppm 2.22 (3 H, s), 2.39-2.43 (4 H, m), 3.38-3.42 (4 H, m), 6.83 (1 H, d, J=9.03 Hz), 7.73 (1 H, s), 7.89 (1 H, s), 7.95 (1 H, dd, J=9.03, 2.74 Hz), 8.00 (1 H, d, J=5.48 Hz), 8.36 (1 H, d, J=5.48 Hz), 8.56 (1 H, d, J=2.74 Hz), 9.03 (1 H, d, J=0.82 Hz), 9.13 (1 H, s), 9.23 (1 H, s), 11.99 (1 H, br s); ESIMS found for C₂₃H₂₂N₈S m/z 443.05 (M+1).

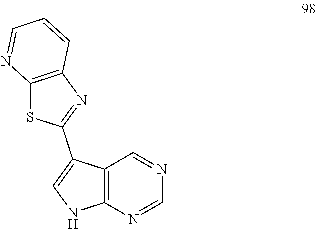 [SMILES: c1cnc2sc(-c3c[nH]c4ncncc34)nc2c1]

2-(7H-Pyrrolo[2,3-d]pyrimidin-5-yOthiazolo[5,4-b]pyridine 98.

Beige solid (18 mg, 0.071 mmol, 96.5% yield). ¹H NMR (499 MHz, DMSO-d₆) δppm 7.58 (1 H, dd, J=8.23, 4.67 Hz), 8.38 (1 H, dd, J=8.10, 1.51 Hz), 8.56 (1 H, dd, J=4.67, 1.65 Hz), 8.62 (1 H, s), 8.94 (1 H, s), 9.66 (1 H, s), 13.01 (1 H, br s); ESIMS found for C₁₂H₇N₅S m/z 254.1 (M+1).

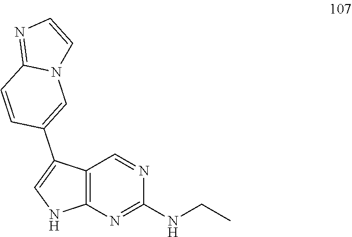 [SMILES: CCNc1ncc2c(-c3ccc4nccn4c3)c[nH]c2n1]

N-Ethyl-5-(imidazo[1,2-a]pyridin-6-yl)-7H-pyrrolo[2,3-d]pyrimidin-2-amine 107.

Beige solid (17 mg, 0.061 mmol, 39.4% yield). ¹H NMR (499 MHz, DMSO-d₆) δppm 1.16 (3 H, t, J=7.14 Hz), 3.29-3.37 (2 H, m), 6.79 (1 H, br t, J=5.35 Hz), 7.53 (1 H, d, J=2.47 Hz), 7.54 (1 H, d, J=1.10 Hz), 7.56-7.59 (1 H, m), 7.60-7.64 (1 H, m), 7.97(1 H, s), 8.92 (1 H, s), 9.00 (1 H, s), 11.51 (1 H, br s); ESIMS found for CisH₁₄N₆ m/z 279.1 (M+1).

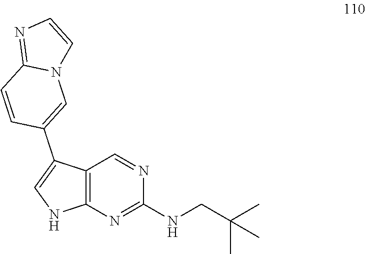 [SMILES: CC(C)(C)CNc1ncc2c(-c3ccc4nccn4c3)c[nH]c2n1]

5-(Imidazo [1,2-a] pyridin-6-yl)-N-ne opentyl-7H-pyrrolo [2,3-d] pyrimidin-2-amine 110.

Beige solid (81 mg, 0.253 mmol, 64.7% yield). ¹H NMR (499 MHz, DMSO-d₆) δppm 0.93 (9 H, s), 3.23 (2 H, d, J=6.57 Hz), 6.72 (1 H, br t, J=5 .7 5 Hz), 7.52 (1 H, d, J=1.64 Hz), 7.54 (1 H, d, J=1.09 Hz), 7.55-7.59 (1 H, m), 7.59-7.63 (1 H, m), 7.97 (1 H, s), 8.91 (1 H, s), 8.98 (1 H, s), 11.46 (1 H, br s); ESIMS found for C₁₈14₂₀1\1₆ m/z 321.2 (M+1).

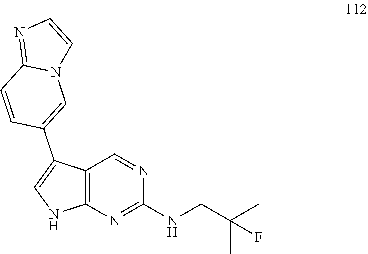 [SMILES: CC(C)(F)CNc1ncc2c(-c3ccc4nccn4c3)c[nH]c2n1]

N-(2-Fluoro-2-methylpropyl)-5-(imidazo[1,2-a]pyridin-6-yl)-7H-pyrrolo [2,3-d] pyrimidin-2-amine 112.

Beige solid (16 mg, 0.049 mmol, 47.2% yield). ¹H NMR (499 MHz, DMSO-d₆) δppm 1.35 (6 H, d, J=21.40 Hz), 3.59 (2 H, dd, J=18.89, 6.30 Hz), 6.90 (1 H, br t, J=6.16 Hz), 7.54 (1 H, d, J=1.10 Hz), 7.55-7.59 (2 H, m), 7.60-7.64 (1 H, m), 7.97 (1 H, s), 8.93 (1 H, s), 9.02 (1 H, s), 11.53 (1 H, br s); ESIMS found for C₁₇H₁₇FN₆ m/z 325.2 (M+1).

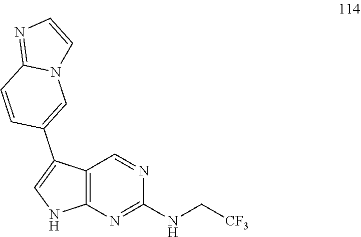 [SMILES: FC(F)(F)CNc1ncc2c(-c3ccc4nccn4c3)c[nH]c2n1]

5-(Imidazo [1,2-a] pyridin-6-yl)-N-(2,2,2-trifluoroethyl)-7H-pyrrolo [2,3-d] pyrimidin-2-amine 114.

White solid (0.86 mg, 0.003 mmol). ¹H NMR (400 MHz, DMSO-d₆) δppm 4.11-4.25 (2 H, m), 7.47 (1 H, br t, J=6.30 Hz), 7.55 (1 H, s), 7.57-7.66(3 H, m), 7.97(1 H, s), 8.96 (1 H, s), 9.09 (1 H, s), 11.73 (1 H, br s); ESIMS found for CisH₁ₗF₃N₆ m/z 333.0 (M+1).

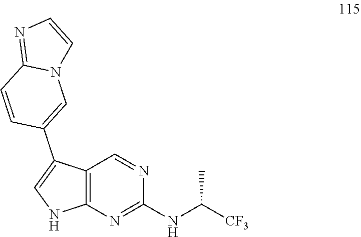 [SMILES: C[C@@H](Nc1ncc2c(-c3ccc4nccn4c3)c[nH]c2n1)C(F)(F)F]

(R)-5-(Imidazo [1,2-a]pyridin-6-yl)-N-(1,1,1-trifluoropropan-2-yl)-7H-pyrrolo [2,3-d] pyrimidin-2-amine 115.

Beige solid (1.4 g, 4.043 mmol, 57.3% yield). ¹H NMR (499 MHz, DMSO-d₆) δppm 1.36 (3 H, d, J=7.12 Hz), 4.88-5.01 (1 H, m), 7.41 (1 H, br d, J=8.76 Hz), 7.55 (1 H, d, J=1.10 Hz), 7.57-7.60 (1 H, m), 7.61-7.65 (1 H, m), 7.63 (1 H, s), 7.97 (1 H, s), 8.95 (1 H, s), 9.08 (1 H, s), 11.67 (1 H, br s); ESIMS found for C₁₆H₁₃F₃N₆ m/z 347.15 (M+1).

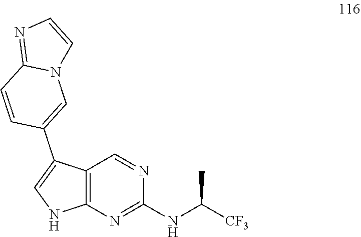 [SMILES: C[C@H](Nc1ncc2c(-c3ccc4nccn4c3)c[nH]c2n1)C(F)(F)F]

(S)-5-(Imidazo [1,2-a]pyridin-6-yl)-N-(1,1,1-trifluoropropan-2-yl)-7H-pyrrolo [2,3-d] pyrimidin-2-amine 116.

White solid (4.72 mg, 0.014 mmol). ¹H NMR (400 MHz, DMSO-d₆) δppm 1.36 (3 H, br d, J=6.97 Hz), 4.95 (1 H, br dd, J=14.24, 6.79 Hz), 7.41 (1 H, br d, J=8.80 Hz), 7.55 (1 H, s), 7.57-7.66 (3 H, m), 7.97 (1 H, s), 8.95 (1 H, s), 9.08 (1 H, s), 11.67 (1 H, br s); ESIMS found for C₁₆K3F3N6 m/z 347.0 (M +1).

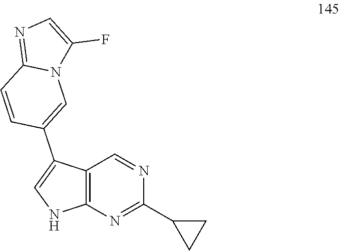 [SMILES: Fc1cnc2ccc(-c3c[nH]c4nc(C5CC5)ncc34)cn12]

2—Cyclopropyl-5-(3-fluoroimidazo [1,2-a]pyridin-6-yl)-7H-pyrrolo [2,3-d] pyrimidine 145.

Off-white solid (9 mg, 0.031 mmol, 11.3% yield). ¹H NMR (499 MHz, DMSO-d₆) δppm 0.98-1.06 (4 H, m), 2.19-2.29 (1 H, m), 7.37 (1 H, d, J=7.12 Hz), 7.56-7.62 (1 H, m), 7.65-7.71 (1 H, m), 7.96 (1 H, s), 8.52 (1 H, s), 9.24 (1 H, s), 12.20 (1 H, br s); ESIMS found for C₁₆H₁₂FN5 m/z 2941. (M+1).

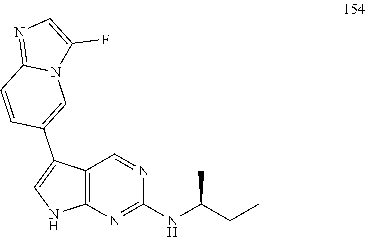 [SMILES: CC[C@H](C)Nc1ncc2c(-c3ccc4ncc(F)n4c3)c[nH]c2n1]

(S)-N-(sec-Butyl)-5-(3-fluoroimidazo [1,2-a]pyridin-6-yl)-7H-pyrrolo [2,3-d] pyrimidin-2-amine 154.

Beige solid (6 mg,0.0185 mmol, 26.3% yield). ¹H NMR (499 MHz, DMSO-d₆) δppm 0.89 (3 H, t, J=7.53 Hz), 1.14 (3 H, d, J=6.57 Hz), 1.41-1.53 (1 H, m), 1.54-1.66 (1 H, m), 3.84-3.96 (1 H, m), 6.60 (1 H, br d, J=7.94 Hz), 7.34 (1 H, d, J=7.39 Hz), 7.54-7.58 (2 H, m), 7.60-7.64 (1 H, m), 8.40 (1 H, s), 8.90 (1 H, s), 11.53 (1 H, br s); ESIMS found for C₁₇H₁₇FN₆ m/z 325.2 (M+1).

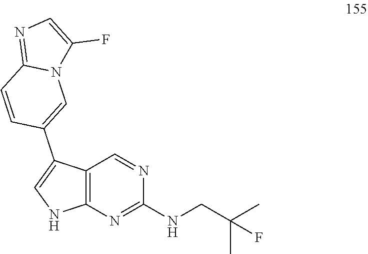 [SMILES: CC(C)(F)CNc1ncc2c(-c3ccc4ncc(F)n4c3)c[nH]c2n1]

N-(2-Fluoro-2-methylpropyl)-5-(3-fluoroimidazo[1,2-a]pyridin-6-yl)-7H-pyrrolo[2,3-d]pyrimidin-2-amine 155.

White solid (13 mg, 0.038 mmol, 25.6% yield). ¹H NMR (499 MHz, DMSO-d₆) δppm 1.35 (6 H, d, J=21.40 Hz), 3.59 (2 H, dd, J=19.03, 6.43 Hz), 6.91 (1 H, br t, J=6.02 Hz), 7.35 (1 H, d, J=7.12 Hz), 7.54-7.64 (3 H, m), 8.41 (1 H, s), 8.93 (1 H, s), 11.58 (1 H, br s); ESIMS found for C₁₇H₁₆F2N6 m/z 343.1 (M+1).

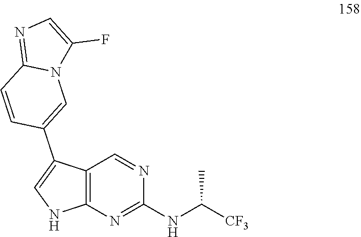 [SMILES: C[C@@H](Nc1ncc2c(-c3ccc4ncc(F)n4c3)c[nH]c2n1)C(F)(F)F]

(R)-5-(3-Fluoroimidazo [1,2-a]pyridin-6-yl)-N-(1,1,1-trifluoropropan-2-yl)-7H-pyrrolo [2,3-d]pyrimidin-2-amine 158.

Pinkish white solid (5 mg, 0.096 mmol, 21.6% yield). ¹H NMR (499 MHz, DMSO-d₆) δppm 1.36 (3 H, d, J=7.12 Hz), 4.89-5.01 (1 H, m), 7.35 (1 H, d, J=7.12 Hz), 7.42 (1 H, br d, J=8.76 Hz), 7.55-7.59 (1 H, m), 7.60-7.65 (1 H, m), 7.66 (1 H, s), 8.44 (1 H, s), 8.99 (1 H, s), 11.73 (1 H, br s); ESIMS found for C₁₆H₁₂F₄N₆ m/z 365.1 (M+1).

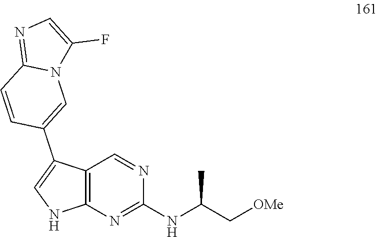 [SMILES: COC[C@H](C)Nc1ncc2c(-c3ccc4ncc(F)n4c3)c[nH]c2n1]

(S)-5-(3-Fluoroimidazo[1,2-a]pyridin-6-yl)-N-(1-methoxypropan-2-yl)-7H-pyrrolo [2,3-d] pyrimidin-2-amine 161.

Light brown solid (7 mg, 0.021 mmol, 21.0% yield). ¹H NMR (499 MHz, DMSO-d₆) δppm 1.16 (3 H, d, J=6.57 Hz), 3.25 (1 H, dd, J=9.17, 6.71 Hz), 3.27 (3 H, s), 3.45 (1 H, dd, J=9.03, 5.48 Hz), 4.18 (1 H, dt, J=13.62, 6.74 Hz), 6.59 (1 H, br d, J=7.94 Hz), 7.35 (1 H, d, J=7.12 Hz), 7.54-7.57 (1 H, m), 7.58 (1 H, d, J=2.19 Hz), 7.60-7.64 (1 H, m), 8.41 (1 H, s), 8.92 (1 H, s), 11.58 (1 H, br s); ESIMS found for C₁₇H₁₇FN₆O m/z 341.2 (M+1).

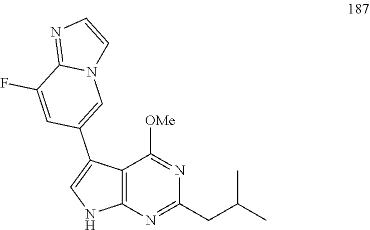 [SMILES: COc1nc(CC(C)C)nc2[nH]cc(-c3cc(F)c4nccn4c3)c12]

5-(8-Fluoroimidazo[1,2-a]pyridin-6-yl)-2-isobutyl-4-methoxy-7H-pyrrolo [2,3-d]pyrimidine 187.

White solid (13.66 mg, 0.040 mmol). ¹H NMR (400 MHz, DMSO-d₆) δppm 0.94 (6 H, d, J=6.60 Hz), 2.27 (1 H, dquin, J=13.66, 6.83, 6.83, 6.83, 6.83 Hz), 2.69 (2 H, d, J=7.21 Hz), 4.05 (3 H, s), 7.51 (1 H, dd, J=12.84, 1.22 Hz), 7.61 (1 H, d, J=0.98 Hz), 7.68 (1 H, s), 8.14 (1 H, dd, J=3.06, 0.98 Hz), 8.77 (1 H, d, J=1.22 Hz), 12.20 (1 H, br s); ESIMS found for C181⁻118FN50 m/z 340.1 (M+1).

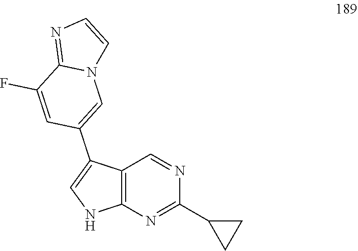 [SMILES: Fc1cc(-c2c[nH]c3nc(C4CC4)ncc23)cn2ccnc12]

2—Cyclopropyl-5-(8-fluoroimidazo [1,2-a] pyridin-6-yl)-7H-pyrrolo [2,3-d] pyrimidine 189.

Off-white solid (4 mg, 0.014 mmol, 14.2% yield). ¹1-1 NMR (499 MHz, DMSO-d₆) δppm 1.00-1.06 (4 H, m), 2.21-2.29 (1 H, m), 7.62 (1 H, d, J=0.82 Hz), 7.66 (1 H, dd, J=12.59, 1.37 Hz), 8.00 (1 H, s), 8.10 (1 H, dd, J=3.01, 0.82 Hz), 8.94 (1 H, d, J=1.37 Hz), 9.35 (1 H, s), 12.20 (1 H, br s); ESIMS found for C₁₆H₁₂FN₅ m/z 294.15 (M+1).

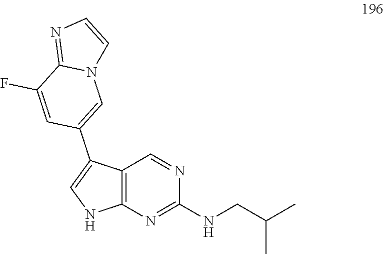 [SMILES: CC(C)CNc1ncc2c(-c3cc(F)c4nccn4c3)c[nH]c2n1]

5-(8-Fluoroimidazo[1,2-a]pyridin-6-yl)-N-isobutyl-7H-pyrrolo[2,3-d] pyrimidin-2-amine 196.

White solid (11 mg, 0.034 mmol, 28.6% yield). ¹H NMR (499 MHz, DMSO-d₆) δppm 0.91 (6 H, d, J=6.84 Hz), 1.86-1.98 (1 H, m), 3.13 (2 H, t, J=6.43 Hz), 6.91 (1 H, br s), 7.57-7.61 (3 H, m), 8.09 (1 H, d, J=2.46 Hz), 8.83 (1 H, d, J=1.10 Hz), 9.02 (1 H, s), 11.53 (1 H, br s); ESIMS found for C₁₇H₁₇FN6 m/z 325.15 (M+1).

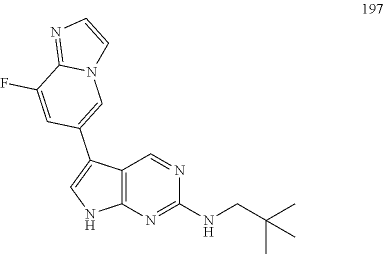 [SMILES: CC(C)(C)CNc1ncc2c(-c3cc(F)c4nccn4c3)c[nH]c2n1]

5-(8-Fluoroimidazo [1,2-a]pyridin-6-yl)-N-neopentyl-7H-pyrrolo [2,3-d] pyrimidin-2-amine 197.

White solid (14.98 mg, 0.044 mmol. ¹H NMR (400 MHz, DMSO-d₆) δppm 0.92 (9 H, s), 3.22 (2 H, br d, J=6.11 Hz), 6.74 (1 H, br s), 7.52-7.65 (3 H, m), 8.09 (1 H, br d, J=1.83 Hz), 8.82 (1 H, s), 9.01 (1 H, s), 11.43 (1 H, br s); ESIMS found for C₁₈H₁₉FN₆ m/z 339.1 (M+1).

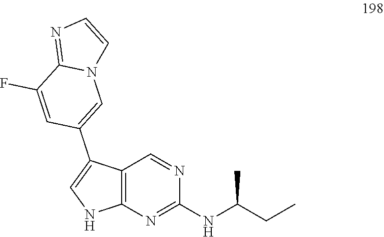 [SMILES: CC[C@H](C)Nc1ncc2c(-c3cc(F)c4nccn4c3)c[nH]c2n1]

(S)-N-(sec-Butyl)-5-(8-fluoroimidazo [1,2-a]pyridin-6-yl)-7H-pyrrolo [2,3-d] pyrimidin-2-amine 198.

White solid (4 mg, 0.012 mmol, 5.1% yield). ¹H NMR (500 MHz, CHLOROFORM-d) δppm 1.00 (3 H, t, J=7.41 Hz), 1.26 (3 H, d, J=6.59 Hz), 1.57-1.63 (2 H, m), 3.99-4.08 (1 H, m), 7.09 (1 H, d, J=1.37 Hz), 7.12 (1 H, dd, J=11.25, 1.37 Hz), 7.69 (1 H, d, J=1.10 Hz), 7.71 (1 H, dd, J=3.02, 1.10 Hz), 8.18 (1 H, d, J=1.37 Hz), 8.43 (1 H, br s), 8.76 (1 H, s); ESIMS found for C₁₇H₁₇FN6 m/z 325.2 (M+1).

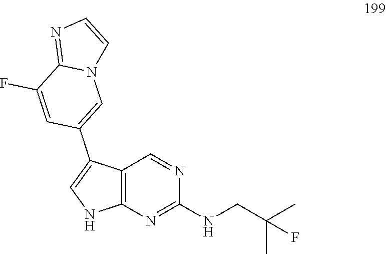 [SMILES: CC(C)(F)CNc1ncc2c(-c3cc(F)c4nccn4c3)c[nH]c2n1]

N-(2-Fluoro-2-methylpropyl)-5-(8-fluoroimidazo[1,2-a]pyridin-6-yl)-7H-pyrrolo [2,3-d] pyrimidin-2-amine 199.

Beige solid (65 mg, 0.190 mmol, 18.7% yield). ¹H NMR (499 MHz, DMSO-d₆) δppm 1.35 (6 H, d, J=21.40 Hz), 3.59 (2 H, dd, J=19.16, 6.57 Hz), 6.92 (1 H, br t, J=6.02 Hz), 7.59 (1 H, br dd, J=12.87, 1.37 Hz), 7.59 (1 H, d, J=1.10 Hz), 7.64 (1 H, d, J=2.19 Hz), 8.09 (1 H, d, J=2.19 Hz), 8.84 (1 H, d, J=1.10 Hz), 9.05 (1 H, s), 11.58 (1 H, br s); ESIMS found for C₁₇H₁₆F2N6 m/z 343.1 (M+1).

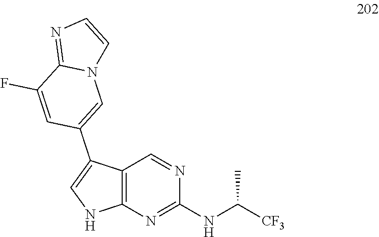 [SMILES: C[C@@H](Nc1ncc2c(-c3cc(F)c4nccn4c3)c[nH]c2n1)C(F)(F)F]

(R)-5-(8-Fluoroimidazo[1,2-a]pyridin-6-yl)-N-(1,1,1-trifluoropropan-2-yl)-7H-pyrrolo[2,3-d]pyrimidin-2-amine 202.

Beige solid (80 mg, 0.220 mmol, 24.1% yield). ¹H NMR (499 MHz, DMSO-d₆) δppm 1.36 (3 H, d, J=6.57 Hz), 4.89-5.02 (1 H, m), 7.44 (1 H, br d, J=8.76 Hz), 7.57-7.65 (2 H, m), 7.71 (1 H, s), 8.09 (1 H, dd, J=3.29, 1.10 Hz), 8.87 (1 H, d, J=1.64 Hz), 9.11 (1 H, s), 11.73 (1 H, br s); ESIMS found for C₁₆H₁₂F₄N₆ m/z 365.1 (M+1).

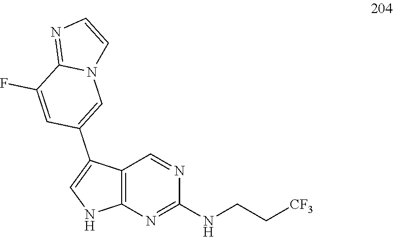 [SMILES: Fc1cc(-c2c[nH]c3nc(NCCC(F)(F)F)ncc23)cn2ccnc12]

5-(8-Fluoroimidazo[1,2-a]pyridin-6-yl)-N-(3,3,3-trifluoropropyl)-7H-pyrrolo [2,3-d]pyrimidin-2-amine 204.

White solid (4 mg, 0.011 mmol, 27.2% yield). ¹H NMR (499 MHz, DMSO-d₆) δppm 2.55-2.67 (2 H, m), 3.51-3.59 (2 H, m), 7.03 (1 H, br s), 7.58-7.62 (2 H, m), 7.66 (1 H, d, J=2.46 Hz), 8.09 (1 H, dd, J=3.01, 1.10 Hz), 8.86 (1 H, d, J=1.10 Hz), 9.09 (1 H, s), 11.65 (1 H, br s); ESIMS found for C₁₆K2F4N6 m/z 365.1 (M+1).

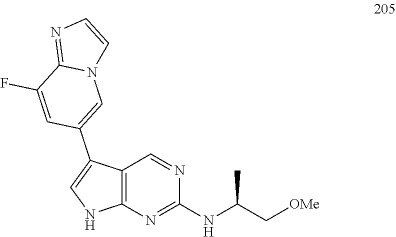 [SMILES: COC[C@H](C)Nc1ncc2c(-c3cc(F)c4nccn4c3)c[nH]c2n1]

(S)-5-(8-Fluoroimidazo[1,2-a]pyridin-6-yl)-N-(1-methoxypropan-2-yl)-7H-pyrrolo [2,3-d] pyrimidin-2-amine 205.

Beige solid (10 mg, 0.029 mmol, 9.3% yield). ¹H NMR (499 MHz, DMSO-d₆) δppm 1.16 (3 H, d, J=6.57 Hz), 3.23-3.27 (1 H, m), 3.27 (3 H, s), 3.45 (1 H, dd, J=9.31, 5.48 Hz), 4.17 (1 H, dt, J=13.62, 6.74 Hz), 6.62 (1 H, br d, J=7.67 Hz), 7.59 (1 H, br dd, J=12.73, 1.23 Hz), 7.59 (1 H, d, J=0.82 Hz), 7.63 (1 H, d, J=2.46 Hz), 8.09 (1 H, dd, J=3.01, 0.82 Hz), 8.84 (1 H, d, J=1.37 Hz), 9.04 (1 H, s), 11.58 (1 H, br s); ESIMS found for C₁₇1-1₁₇FN₆O m/z 341.1 (M+1).

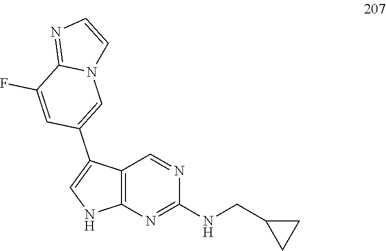 [SMILES: Fc1cc(-c2c[nH]c3nc(NCC4CC4)ncc23)cn2ccnc12]

N-(Cyclopropylmethyl)-5-(8-fluoroimidazo[1,2-a]pyridin-6-yl)-7H-pyrrolo [2,3-d]pyrimidin-2-amine 207.

White solid (6 mg, 0.019 mmol, 15.4% yield). ¹1-1 NMR (499 MHz, DMSO-d₆) δppm 0.20-0.27(2 H, m), 0.38-0.46 (2 H, m), 1.06-1.17 (1 H, m), 3.19 (2 H, t, J=6.30 Hz), 6.91 (1 H, br s), 7.57-7.61 (2 H, m), 7.61 (1 H, d, J=2.46 Hz), 8.09 (1 H, dd, J=3.01, 1.10 Hz), 8.84 (1 H, d, J=1.37 Hz), 9.03 (1 H, s), 11.55 (1 H, br s); ESIMS found for C₁₇HisFN₆ m/z 323.1 (M+1).

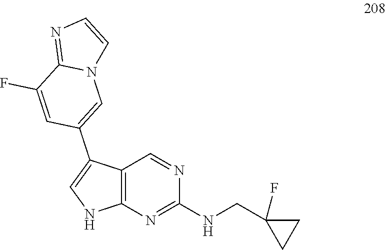 [SMILES: Fc1cc(-c2c[nH]c3nc(NCC4(F)CC4)ncc23)cn2ccnc12]

N-((l-Fluorocyclopropyl)methyl)-5-(8-fluoroimidazo [1,2-a]pyridin-6-yl)-7H-pyrrolo [2,3-d]pyrimidin-2-amine 208.

Tan solid (5 mg, 0.015 mmol, 17.7% yield). ¹H NMR (500 MHz, DMSO-d₆) δppm 0.77-0.87 (2 H, m), 0.91-1.02 (2 H, m), 3.81 (2 H, dd, J=19.49, 6.04 Hz), 7.09 (1 H, br t, J=5.63 Hz), 7.57-7.62 (2 H, m), 7.64 (1 H, d, J=2.47 Hz), 8.09 (1 H, dd, J=3.02, 1.10 Hz), 8.85 (1 H, d, J=1.37 Hz), 9.05 (1 H, s), 11.59 (1 H, br s); ESIMS found for C₁₇H₁₄F₂N₆ m/z 341.1 (M+1).

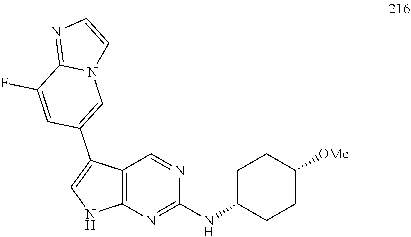 [SMILES: CO[C@H]1CC[C@@H](Nc2ncc3c(-c4cc(F)c5nccn5c4)c[nH]c3n2)CC1]

5-(8-Fluoroimidazo[1,2-a]pyridin-6-yl)-N-(cis-4-methoxycyclohexyl)-7H-pyrrolo[2,3-d]pyrimidin-2-amine 216.

White solid (7.13 mg, 0.019 mmol). ¹H NMR (400 MHz, DMSO-d₆) δppm 1.54-1.68 (4 H, m), 1.71 (2 H, br d, J=4.50 Hz), 1.77-1.85 (2 H, m), 1.93-2.05 (1 H, m), 3.24 (3 H, s), 3.86-3.97 (1 H, m), 7.83-8.07 (1 H, m), 7.84-7.91 (2 H, m), 8.03 (1 H, s), 8.17 (1 H, s), 8.98 (1 H, s), 9.25 (1 H, s), 12.61 (1 H, br s); ESIMS found for C20H_{2,}FN6O m/z 381.0 (M+1).

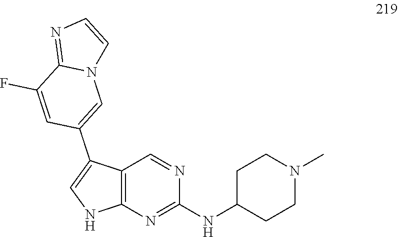 [SMILES: CN1CCC(Nc2ncc3c(-c4cc(F)c5nccn5c4)c[nH]c3n2)CC1]

5-(8-Fluoroimidazo[1,2-a]pyridin-6-yl)-N-(1-methylpiperidin-4-yl)-7H-pyrrolo [2,3-d] pyrimidin-2-amine 219.

White solid (4.69 mg, 0.013 mmol). ¹H NMR (400 MHz, DMSO-d₆) δppm 1.49-1.64 (2 H, m), 1.89 (2 H, br dd, J=11.51, 1.50 Hz), 1.95-2.07 (2 H, m), 2.20 (3 H, s), 2.79 (2 H, br d, J=11.76 Hz), 3.64-3.79 (1 H, m), 6.73 (1 H, br d, J=7.38 Hz), 7.56-7.62 (3 H, m), 8.09 (1 H, d, J=2.63 Hz), 8.83 (1 H, s), 9.04 (1 H, s), 11.57 (1 H, br s); ESIMS found for C₁₉H₂₀FN₇ m/z 366.1 (M+1).

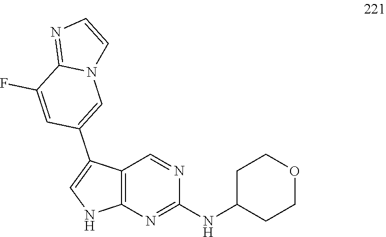 [SMILES: Fc1cc(-c2c[nH]c3nc(NC4CCOCC4)ncc23)cn2ccnc12]

5-(8-Fluoroimidazo[1,2-a]pyridin-6-yl)-N-(tetrahydro-2H-pyran-4-yl)-7H-pyrrolo [2,3-d] pyrimidin-2-amine 221.

White solid (11.07 mg, 0.031 mmol). ¹H NMR (400 MHz, DMSO-d₆) δppm 1.46-1.60 (2 H, m), 1.88 (2 H, br dd, J=12.41, 2.14 Hz), 3.42 (2 H, br s), 3.85-3.92 (2 H, m), 3.92-4.01 (1 H, m), 6.83 (1 H, br d, J=7.70 Hz), 7.58 (1 H, br dd, J=12.78, 1.16 Hz), 7.59 (1 H, d, J=0.98 Hz), 7.61 (1 H, s), 8.09 (1 H, dd, J=3.00, 0.92 Hz), 8.83 (1 H, d, J=1.22 Hz), 9.04 (1 H, s); ESIMS found for C₁₈H₁₇FN60 m/z 353.1 (M+1).

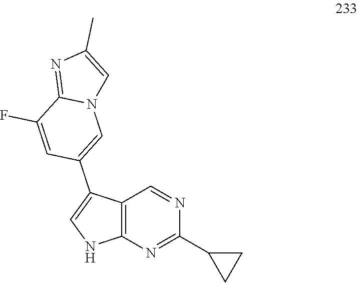 [SMILES: Cc1cn2cc(-c3c[nH]c4nc(C5CC5)ncc34)cc(F)c2n1]

2—Cyclopropyl-5-(8-fluoro-2-methylimidazo[1,2-a]pyridin-6-yl)-7H-pyrrolo [2,3-d]pyrimidine 233.

Light brown solid (4 mg, 0.013 mmol, 3.7% yield). ¹1-1 NMR (499 MHz, DMSO-d₆) δppm 1.00-1.06 (4 H, m), 2.20-2.29 (1 H, m), 2.37 (3 H, s), 7.58 (1 H, dd, J=12.62, 1.37 Hz), 7.83 (1 H, d, J=2.47 Hz), 7.96 (1 H, s), 8.83 (1 H, d, J=1.37 Hz), 9.33 (1 H, s), 12.17 (1 H, br s); ESIMS found for C₁₇H₁₄FN₅ m/z 308.1 (M+1).

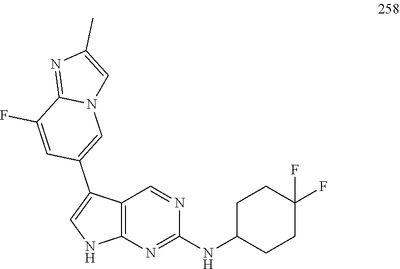 [SMILES: Cc1cn2cc(-c3c[nH]c4nc(NC5CCC(F)(F)CC5)ncc34)cc(F)c2n1]

N-(4,4-Difluorocyclohexyl)-5-(8-fluoro-2-methylimidazo [1,2-a]pyridin-6-yl)-7H-pyrrolo [2,3-d] pyrimidin-2-amine 258.

Off-white solid (3.5 mg, 0.009 mmol, 9.0% yield). ¹1-1 NMR (499 MHz, DMSO-d₆) δppm 1.58-1.68 (2 H, m), 1.86-2.02 (4 H, m), 2.04-2.16 (2 H, m), 2.36 (3 H, d, J=0.82 Hz), 3.88-4.00 (1 H, m), 6.90 (1 H, br d, J=7.39 Hz), 7.52 (1 H, dd, J=12.87, 1.37 Hz), 7.59 (1 H, s), 7.80-7.85 (1 H, m), 8.73 (1 H, d, J=1.37 Hz), 9.03 (1 H, s), 11.57 (1 H, br s); ESIMS found for C₂₀H₁₉F3N6 m/z 401.2 (M+1).

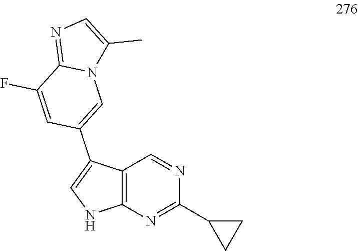 [SMILES: Cc1cnc2c(F)cc(-c3c[nH]c4nc(C5CC5)ncc34)cn12]

2—Cyclopropyl-5-(8-fluoro-3-methylimidazo[1,2-a]pyridin-6-yl)-7H-pyrrolo [2,3-d]pyrimidine 276.

Off-white solid (12.3 mg, 0.040 mmol, 21.2% yield). ¹1-1 NMR (499 MHz, DMSO-d₆) δppm 1.00-1.06 (4 H, m), 2.20-2.29 (1 H, m), 2.58 (3 H, s), 7.42 (1 H, s), 7.59 (1 H, d, J=12.62 Hz), 7.97 (1 H, s), 8.35 (1 H, d, J=0.82 Hz), 9.30 (1 H, s), 12.19 (1 H, br s); ESIMS found for C₁₇H₁₄FN₅ m/z 308.1 (M+1).

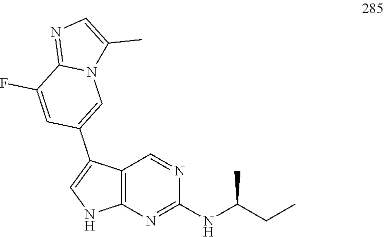 [SMILES: CC[C@H](C)Nc1ncc2c(-c3cc(F)c4ncc(C)n4c3)c[nH]c2n1]

(S)-N-(sec-Butyl)-5-(8-fluoro-3-methylimidazo[1,2-a]pyridin-6-yl)-7H-pyrrolo [2,3-d]pyrimidin-2-amine 285.

Beige solid (7 mg, 0.021 mmol, 30.0% yield). ¹H NMR (499 MHz, DMSO-d₆) δppm 0.89 (3 H, t, J=7.39 Hz), 1.14 (3 H, d, J=6.57 Hz), 1.43-1.53 (1 H, m), 1.54-1.65 (1 H, m), 2.57 (3 H, s), 3.92 (1 H, dt, J=14.24, 6.84 Hz), 6.57 (1 H, br d, J=8.21 Hz), 7.39 (1 H, s), 7.52 (1 H, dd, J=12.59, 1.10 Hz), 7.57 (1 H, d, J=2.19 Hz), 8.23 (1 H, d, J=1.37 Hz), 8.95 (1 H, s), 11.51 (1 H, br s); ESIMS found for CisH₁₉FN6 m/z 339.2 (M+1).

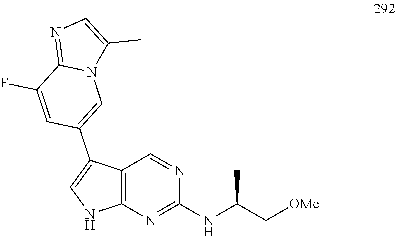 [SMILES: COC[C@H](C)Nc1ncc2c(-c3cc(F)c4ncc(C)n4c3)c[nH]c2n1]

(S)-5-(8-Fluoro-3-methylimidazo[1,2-a]pyridin-6-yl)-N-(1-methoxypropan-2-yl)-7H-pyrrolo[2,3-d]pyrimidin-2-amine 292.

Beige solid (12 mg, 0.034 mmol, 30.8% yield). ¹H NMR (499 MHz, DMSO-d₆) δppm 1.17 (3 H, d, J=6.84 Hz), 2.57 (3 H, s), 3.23-3.27 (1 H, m), 3.27 (3 H, s), 3.45 (1 H, dd, J=9.31, 5.48 Hz), 4.19 (1 H, dt, J=13.62, 6.74 Hz), 6.56 (1 H, br d, J=8.21 Hz), 7.39 (1 H, s), 7.53 (1 H, dd, J=12.59, 0.82 Hz), 7.59 (1 H, d, J=2.19 Hz), 8.24 (1 H, d, J=1.10 Hz), 8.98 (1 H, s), 11.58 (1 H, br s); ESIMS found for C₁₈H₁₉FN₆O m/z 355.2 (M+1).

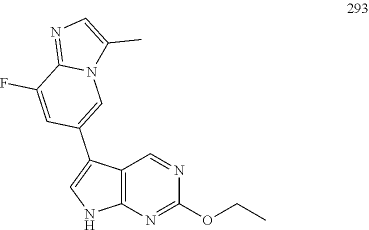 [SMILES: CCOc1ncc2c(-c3cc(F)c4ncc(C)n4c3)c[nH]c2n1]

2-Ethoxy-5-(8-fluoro-3-methylimidazo[1,2-a]pyridin-6-yl)-7H-pyrrolo[2,3-d] pyrimidine 293.

Off-white solid (12.2 mg, 0.039 mmol, 18.2% yield). ¹1-1 NMR (499 MHz, DMSO-d₆) δppm 1.37 (3 H, t, J=7.14 Hz), 2.58 (3 H, s), 4.39 (2 H, q, J=7.04 Hz), 7.41 (1 H, s), 7.58 (1 H, d, J=12.62 Hz), 7.88 (1 H, s), 8.34 (1 H, s), 9.23 (1 H, s), 12.14 (1 H, br s); ESIMS found for C₁₆H₁₄FN50 m/z 312.0 (M+1).

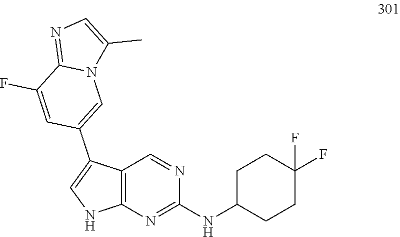 [SMILES: Cc1cnc2c(F)cc(-c3c[nH]c4nc(NC5CCC(F)(F)CC5)ncc34)cn12]

N-(4,4-Difluorocyclohexyl)-5-(8-fluoro-3-methylimidazo [1,2-a]pyridin-6-yl)-7H-pyrrolo [2,3-d] pyrimidin-2-amine 301.

Off-white solid (4.5 mg, 0.011 mmol, 17.8% yield). ¹H NMR (499 MHz, DMSO-d₆) δppm 1.58-1.70 (2 H, m), 1.84-2.02 (4 H, m), 2.04-2.16 (2 H, m), 2.57 (3 H, s), 3.90-4.02 (1 H, m), 6.86 (1 H, br d, J=7.67 Hz), 7.40 (1 H, d, J=0.82 Hz), 7.53 (1 H, br dd, J=12.59, 1.37 Hz), 7.59 (1 H, s), 8.24 (1 H, d, J=1.10 Hz), 8.99 (1 H, s); ESIMS found for C₂₀H₁₉F3N6 m/z 401.1 (M+1).

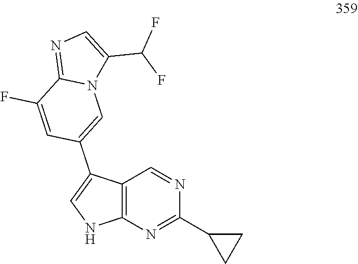 [SMILES: Fc1cc(-c2c[nH]c3nc(C4CC4)ncc23)cn2c(C(F)F)cnc12]

2—Cyclopropyl-5-(3-(difluoromethyl)-8-fluoroimidazo [1,2-a]pyridin-6-yl)-7H-pyrrolo [2,3-d]pyrimidine 359.

Olive green solid (12 mg, 0.035 mmol, 21.7% yield). ¹H NMR (499 MHz, DMSO-d₆) δppm 1.00-1.06 (4 H, m), 2.20-2.30 (1 H, m), 7.73 (1 H, t, J=53.50 Hz), 7.87 (1 H, d, J=12.32 Hz), 7.99 (1 H, s), 8.04 (1 H, d, J=2.46 Hz), 8.59 (1 H, d, J=1.10 Hz), 9.19 (1 H, s), 12.30 (1 H, br s); ESIMS found for C₁₇F1₁₂F₃N₅ m/z 344.1 (M+1).

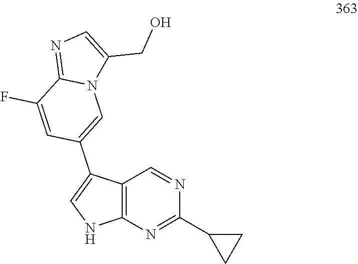 [SMILES: OCc1cnc2c(F)cc(-c3c[nH]c4nc(C5CC5)ncc34)cn12]

(6-(2—Cyclopropyl-7H-pyrrolo[2,3-d]pyrimidin-5-yl)-8-fluoroimidazo[1,2-a] pyridin-3-yl)methanol 363.

Off-white solid (9 mg, 0.028 mmol, 39.1% yield). ¹1-1 NMR (499 MHz, DMSO-d₆) δppm 1.00-1.08 (4 H, m), 2.20-2.30 (1 H, m), 4.92 (2 H, d, J=4.39 Hz), 5.45 (1 H, br t, J=5.08 Hz), 7.56 (1 H, s), 7.67 (1 H, dd, J=12.49, 0.96 Hz), 7.99 (1 H, s), 8.56 (1 H, d, J=1.10 Hz), 9.26 (1 H, s), 12.24 (1 H, br s); ESIMS found for C₁₇H₁₄FN₅O m/z 324.0 (M+1).

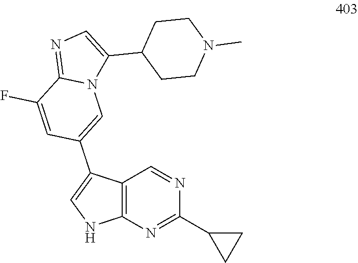 [SMILES: CN1CCC(c2cnc3c(F)cc(-c4c[nH]c5nc(C6CC6)ncc45)cn23)CC1]

2—Cyclopropyl-5-(8-fluoro-3-(1-methylpiperidin-4-yl)imidazo[1,2-a]pyridin-6-yl)-7H-pyrrolo [2,3-d] pyrimidine 403.

Off-white solid (35 mg, 0.090 mmol, 29.6% yield). ¹1-1 NMR (499 MHz, DMSO-d₆) δppm 0.99-1.07 (4 H, m), 1.69 (2 H, qd, J=12.23, 3.56 Hz), 2.02 (2 H, br d, J=12.32 Hz), 2.14 (2 H, td, J=11.64, 1.92 Hz), 2.21-2.26 (1 H, m), 2.23 (3 H, s), 2.86 (2 H, br d, J=11.50 Hz), 3.20 (1 H, tt, J=11.77, 3.56 Hz), 7.45 (1 H, s), 7.56 (1 H, dd, J=12.32, 1.10 Hz), 7.96 (1 H, s), 8.43 (1 H, d, J=1.37 Hz), 9.20 (1 H, s), 12.21 (1 H, br s); ESIMS found for C₂₂H₂₃FN₆ m/z 391.2 (M+1).

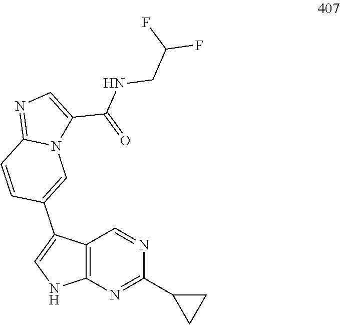 [SMILES: O=C(NCC(F)F)c1cnc2ccc(-c3c[nH]c4nc(C5CC5)ncc34)cn12]

6-(2—Cyclopropyl-7H-pyrrolo[2,3-d]pyrimidin-5-yl)-N-(2,2-difluoroethyl) imidazo[1,2-a]pyridine-3-carboxamide 407.

Beige solid (16 mg, 0.042 mmol, 18.0% yield). ¹H NMR (499 MHz, DMSO-d₆) δppm 1.00-1.08 (4 H, m), 2.21-2.29 (1 H, m), 3.76 (2 H, tdd, J=15.71, 15.71, 5.82, 4.11 Hz), 6.18 (1 H, tt, J=56.00, 3.90 Hz), 7.81 (1 H, d, J=9.31 Hz), 7.94 (1 H, dd, J=9.31, 1.92 Hz), 8.04 (1 H, s), 8.42 (1 H, s), 8.91 (1 H, t, J=5.89 Hz), 9.16 (1 H, s), 9.93 (1 H, d, J=0.82 Hz), 12.25 (1 H, br s); ESIMS found for C₁₉H₁₆F2N60 m/z 383.2 (M+1).

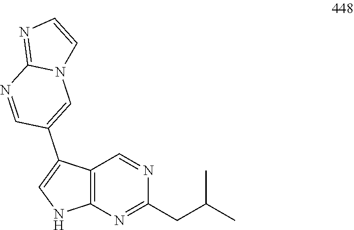 [SMILES: CC(C)Cc1ncc2c(-c3cnc4nccn4c3)c[nH]c2n1]

5-(Imidazo[1,2-a]pyrimidin-6-yl)-2-isobutyl-7H-pyrrolo[2,3-d]pyrimidine 448.

White solid (110 mg, 0.376 mmol, 37.5% yield). ¹H NMR (499 MHz, DMSO-d₆) δppm 0.93 (6 H, d, J=6.57 Hz), 2.28 (1 H, dquin, J=13.54, 6.88, 6.88, 6.88, 6.88 Hz), 2.81 (2 H, d, J=7.12 Hz), 7.73 (1 H, d, J=1.09 Hz), 7.92 (1 H, d, J=1.37 Hz), 8.13 (1 H, s), 9.02 (1 H, d, J=2.74 Hz), 9.40 (1 H, s), 9.43 (1 H, d, J=2.46 Hz), 12.34 (1 H, br s); ESIMS found for C₁₆H₁₆N₆ m/z 293.2 (M+1).

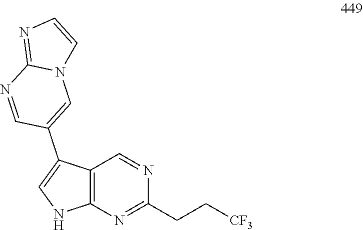 [SMILES: FC(F)(F)CCc1ncc2c(-c3cnc4nccn4c3)c[nH]c2n1]

5-(Imidazo[1,2-a]pyrimidin-6-yl)-2-(3,3,3-trifluoropropyl)-7H-pyrrolo[2,3-d]pyrimidine 449.

Off-white solid (15 mg, 0.045 mmol, 44.4% yield). ¹H NMR (499 MHz, DMSO-d₆) δppm 2.79-2.94 (2 H, m), 3.21 (2 H, t, J=7.80 Hz), 7.74 (1 H, d, J=1.10 Hz), 7.93 (1 H, d, J=1.37 Hz), 8.18 (1 H, s), 9.02 (1 H, d, J=2.46 Hz), 9.40-9.47 (2 H, m), 12.44 (1 H, br s); ESIMS found for C151⁻111F3N6 m/z 333.1 (M+1).

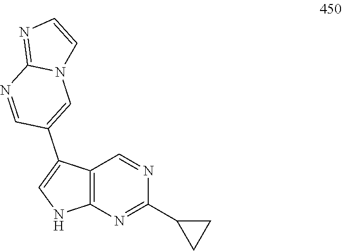 [SMILES: c1cn2cc(-c3c[nH]c4nc(C5CC5)ncc34)cnc2n1]

2—Cyclopropyl-5-(imidazo[1,2-a]pyrimidin-6-yl)-7H-pyrrolo[2,3-d] pyrimidine 450.

Light brown solid (10 mg, 0.036 mmol, 11.1% yield). ¹1-1 NMR (499 MHz, DMSO-d₆) δppm 0.99-1.07 (4 H, m), 2.19-2.31 (1 H, m), 7.73 (1 H, s), 7.91(1 H, s), 8.08(1 H, s), 9.01 (1 H, d, J=2.46 Hz), 9.32 (1 H, s), 9.40 (1 H, d, J=2.46 Hz), 12.26 (1 H, br s); ESIMS found for Cistli2N6 m/z 277.1 (M+1).

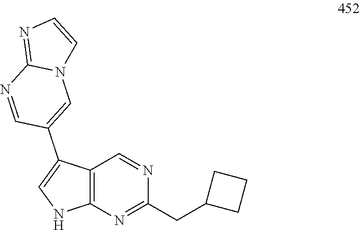 [SMILES: c1cn2cc(-c3c[nH]c4nc(CC5CCC5)ncc34)cnc2n1]

2—(Cyclobutylmethyl)-5-(imidazo [1,2-a] pyrimidin-6-yl)-7H-pyrrolo [2,3-d] pyrimidine 452.

Off-white solid (18 mg, 0.059 mmol, 59.8% yield). ¹H NMR (499 MHz, DMSO-d₆) δppm 1.73-1.91 (4 H, m), 1.99-2.09 (2 H, m), 2.81-2.92 (1 H, m), 3.03 (2 H, d, J=7.67 Hz), 7.73 (1 H, d, J=1.37 Hz), 7.92 (1 H, d, J=1.37 Hz), 8.12 (1 H, s), 9.01 (1 H, d, J=2.46 Hz), 9.38 (1 H, s), 9.42 (1 H, d, J=2.74 Hz), 12.33 (1 H, br s); ESIMS found for C₁₇H₁₆N6 m/z 305.2 (M+1).

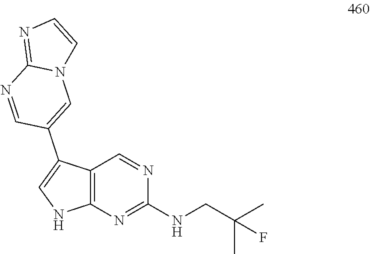 [SMILES: CC(C)(F)CNc1ncc2c(-c3cnc4nccn4c3)c[nH]c2n1]

N-(2-Fluoro-2-methylpropyl)-5-(imidazo [1,2-a] pyrimidin-6-yl)-7H-pyrrolo [2,3-d]pyrimidin-2-amine 460.

Beige solid (6 mg, 0.018 mmol, 29.5% yield). ¹H NMR (499 MHz, DMSO-d₆) δppm 1.35 (6 H, d, J=21.40 Hz), 3.59 (2 H, dd, J=18.89, 6.57 Hz), 6.95 (1 H, br t, J=5.75 Hz), 7.70 (1 H, d, J=1.37 Hz), 7.72 (1 H, d, J=2.46 Hz), 7.90 (1 H, d, J=1.37 Hz), 8.96 (1 H, d, J=2.74 Hz), 9.02 (1 H, s), 9.31 (1 H, d, J=2.74 Hz), 11.65 (1 H, br s); ESIMS found for C₁₆H₁₆FN₇ m/z 326.1 (M+1).

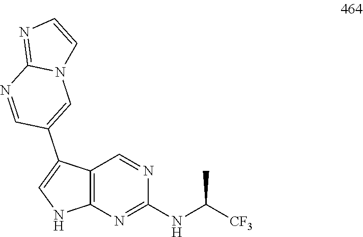 [SMILES: C[C@H](Nc1ncc2c(-c3cnc4nccn4c3)c[nH]c2n1)C(F)(F)F]

(S)-5-(Imidazo[1,2-a]pyrimidin-6-yl)-N-(1,1,1-trifluoropropan-2-yl)-7H-pyrrolo[2,3-d]pyrimidin-2-amine 464.

White solid (17.31 mg, 0.050 mmol). ¹H NMR (400 MHz, DMSO-d₆) δppm 1.36 (3 H, d, J=7.09 Hz), 4.86-5.03 (1 H, m), 7.46 (1 H, br d, J=9.05 Hz), 7.71 (1 H, d, J=1.22 Hz), 7.79 (1 H, s), 7.90 (1 H, d, J=1.34 Hz), 8.97 (1 H, d, J=2.57 Hz), 9.08 (1 H, s), 9.33 (1 H, d, J=2.57 Hz), 11.81 (1 H, br s); ESIMS found for CisH₁₂F₃N₇ m/z 348.1 (M+1).

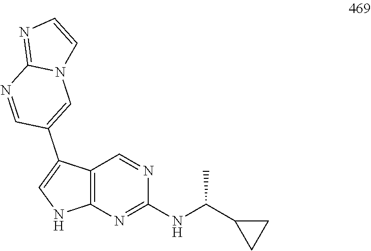 [SMILES: C[C@@H](Nc1ncc2c(-c3cnc4nccn4c3)c[nH]c2n1)C1CC1]

(R)-N-(1-cyclopropylethyl)-5-(imidazo[1,2-a]pyrimidin-6-yl)-7H-pyrrolo [2,3-d]pyrimidin-2-amine 469.

Beige solid (4 mg, 0.013 mmol, 16.0% yield). ¹H NMR (499 MHz, DMSO-d₆) δppm 0.12-0.21 (1 H, m), 0.33-0.38 (2 H, m), 0.39-0.45 (1 H, m), 0.96-1.06(1 H, m), 1.22(3 H, d, J=6.57 Hz), 3.58 (1 H, dq, J=14.75, 7.22 Hz), 6.75 (1 H, br d, J=8.21 Hz), 7.68 (1 H, d, J=2.46 Hz), 7.69 (1 H, d, J=1.37 Hz), 7.90 (1 H, d, J=1.37 Hz), 8.96 (1 H, d, J=2.46 Hz), 8.98 (1 H, s), 9.29 (1 H, d, J=2.46 Hz), 11.57 (1 H, br s); ESIMS found for C₁₇H₁₇N₇ m/z 320.2 (M+1).

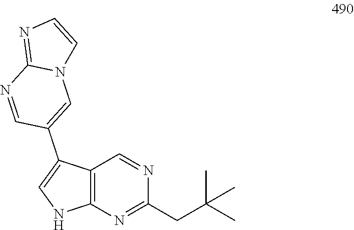 [SMILES: CC(C)(C)Cc1ncc2c(-c3cnc4nccn4c3)c[nH]c2n1]

5-(Imidazo[1,2-a]pyrimidin-6-yl)-2-neopentyl-7H-pyrrolo[2,3-d]pyrimidine 490.

Off-white solid (31 mg, 0.101 mmol, 34.2% yield). ¹H NMR (499 MHz, DMSO-d₆) δppm 0.99 (9 H, s), 2.84 (2 H, s), 7.73 (1 H, d, J=1.37 Hz), 7.92 (1 H, d, J=1.10 Hz), 8.14 (1 H, s), 9.03 (1 H, d, J=2.46 Hz), 9.42 (1 H, s), 9.45 (1 H, d, J=2.46 Hz), 12.37 (1 H, br s); ESIMS found for C₁₇H₁₈N₆ m/z 307.2 (M+1).

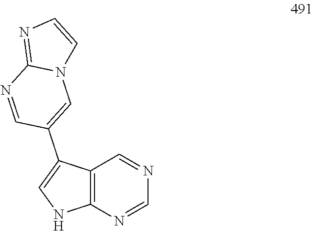 [SMILES: c1ncc2c(-c3cnc4nccn4c3)c[nH]c2n1]

5-(Imidazo[1,2-a]pyrazin-6-yl)-7H-pyrrolo[2,3-d]pyrimidine 491.

Beige solid (35 mg, 0.148 mmol, 48.2% yield). ¹H NMR (499 MHz, DMSO-d₆) δppm 7.84 (1 H, d, J=1.10 Hz), 8.13 (1 H, t, J=0.82 Hz), 8.17 (1 H, s), 8.84 (1 H, s), 9.14-9.17 (1 H, m), 9.19 (1 H, d, J=1.64 Hz), 9.62 (1 H, s), 12.46 (1 H, br s); ESIMS found for C₁₂H₈N6 m/z 237.1 (M+1).

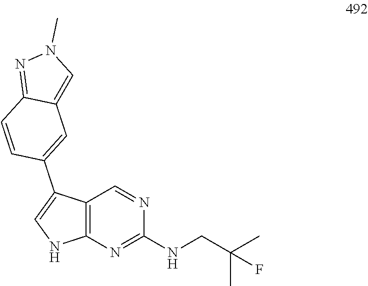 [SMILES: Cn1cc2cc(-c3c[nH]c4nc(NCC(C)(C)F)ncc34)ccc2n1]

N-(2-Fluoro-2-methylpropyl)-5-(2-methyl-2H-indazol-5-yl)-7H-pyrrolo [2,3-d]pyrimidin-2-amine 492.

Beige solid (18 mg, 0.053 mmol, 26.2% yield). ¹H NMR (499 MHz, DMSO-d₆) δppm 1.35 (6 H, d, J=21.40 Hz), 3.59 (2 H, dd, J=18.89, 6.30 Hz), 4.16 (3 H, s), 6.81 (1 H, br t, J=5.75 Hz), 7.42 (1 H, d, J=1.64 Hz), 7.54-7.64 (2 H, m), 7.96 (1 H, s), 8.27 (1 H, s), 8.92 (1 H, s), 11.39 (1 H, br s); ESIMS found for C₁₈H₁₉FN₆m/z 339.2 (M+1).

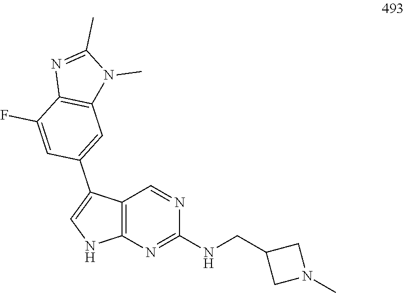 [SMILES: Cc1nc2c(F)cc(-c3c[nH]c4nc(NCC5CN(C)C5)ncc34)cc2n1C]

5-(4-Fluoro-1,2-dimethyl-1H-benzo [d] imidazol-6-yl)-N-((1-methylazetidin-3-yl)methyl)-7H-pyrrolo [2,3-d]pyrimidin-2-amine 493.

Beige solid (9 mg, 0.024 mmol, 50.6% yield). ¹H NMR (499 MHz, DMSO-d₆) δppm 2.31 (3 H, s), 2.33-2.39 (1 H, m), 2.52-2.59 (2 H, m), 2.54 (3 H, s), 3.15 (1 H, br dd, J=11.80, 8.51 Hz), 3.44-3.52 (1 H, m), 3.81 (3 H, s), 4.12 (1 H, dd, J=12.76, 9.19 Hz), 4.52 (1 H, br dd, J=12.90, 3.29 Hz), 7.24-7.30 (1 H, m), 7.48 (1 H, d, J=0.82 Hz), 7.90 (1 H, s), 8.83 (1 H, s); ESIMS found for C20H₂₂FN7 m/z 380.1 (M+1).

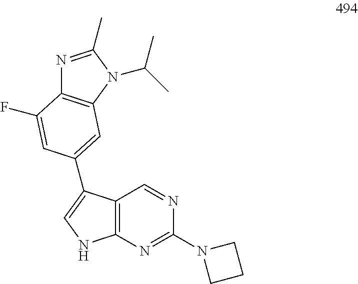 [SMILES: Cc1nc2c(F)cc(-c3c[nH]c4nc(N5CCC5)ncc34)cc2n1C(C)C]

2-(Azetidin-1-yl)-5-(4-fluoro-1-isopropyl-2-methyl-1H-benzo [d] imidazol-6-yl)-7H-pyrrolo [2,3-d] pyrimidine 494.

White solid (15 mg, 0.041 mmol, 31.9% yield). ¹H NMR (499 MHz, DMSO-d₆) δppm 1.60 (6 H, d, J=6.86 Hz), 2.26-2.35 (2 H, m), 2.58 (3 H, s), 4.04 (4 H, t, J=7.41 Hz), 4.82 (1 H, quip, J=6.93 Hz), 7.27 (1 H, dd, J=12.08, 0.82 Hz), 7.58 (1 H, s), 7.63 (1 H, d, J=1.10 Hz), 8.92 (1 H, s), 11.64 (1 H, s); ESIMS found for C20H_{2,}FN6 m/z 365.2 (M+1).

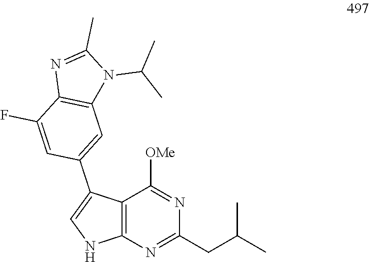 [SMILES: COc1nc(CC(C)C)nc2[nH]cc(-c3cc(F)c4nc(C)n(C(C)C)c4c3)c12]

5-(4-Fluoro-1-isopropyl-2-methyl-1H-benzo[d]imidazol-6-yl)-2-isobutyl-4-methoxy-7H-pyrrolo [2,3-d]pyrimidine 497.

White solid (18.48 mg, 0.047 mmol). ¹H NMR (400 MHz, DMSO-d₆) δppm 0.95 (6 H, d, J=6.60 Hz), 1.61 (6 H, d, J=6.85 Hz), 2.28 (1 H, dquin, J=13.46, 6.66, 6.66, 6.66, 6.66 Hz), 2.58 (3 H, s), 2.68 (2 H, d, J=7.21 Hz), 4.01 (3 H, s), 4.77 (1 H, spt, J=6.70 Hz), 7.26 (1 H, d, J=12.35 Hz), 7.60 (1 H, s), 7.83 (1 H, s), 12.10 (1 H, br s); ESIMS found for C₂₂H₂₆FN₅O m/z 396.2 (M+1).

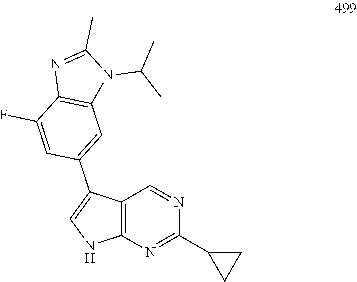 [SMILES: Cc1nc2c(F)cc(-c3c[nH]c4nc(C5CC5)ncc34)cc2n1C(C)C]

2—Cyclopropyl-5-(4-fluoro-1-isopropyl-2-methyl-1H-benzo[d]imidazol-6-yl)-7H-pyrrolo[2,3-d]pyrimidine 499.

White solid (10.4 mg, 0.030 mmol, 12.8% yield). ¹HNMR (499 MHz, DMSO-d₆) δppm 0.97-1.05 (4 H, m), 1.61 (6 H, d, J=6.86 Hz), 2.19-2.29 (1 H, m), 2.59 (3 H, s), 4.84 (1 H, quip, J=6.93 Hz), 7.33 (1 H, dd, J=11.80, 1.10 Hz), 7.70 (1 H, d, J=1.10 Hz), 7.90 (1 H, d, J=1.92 Hz), 9.16 (1 H, s), 12.10 (1 H, s); ESIMS found for C20H₂₀FN5 m/z 350.2 (M+1).

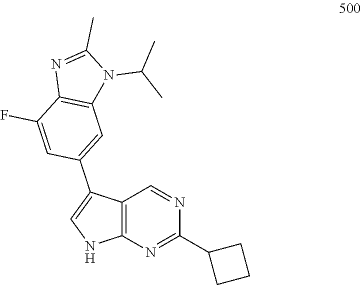 [SMILES: Cc1nc2c(F)cc(-c3c[nH]c4nc(C5CCC5)ncc34)cc2n1C(C)C]

2—Cyclobutyl-5-(4-fluoro-14 sopropyl-2-methyl-1H-benzo [d] imidazol-6-yl)-7H-pyrrolo [2,3-d]pyrimidine 500.

Beige solid (50 mg, 0.138 mmol, 79.1% yield). ¹H NMR (499 MHz, DMSO-d₆) δppm 1.61 (6 H, d, J=7.12 Hz), 1.83-1.94 (1 H, m), 1.97-2.11 (1 H, m), 2.28-2.38 (2 H, m), 2.39-2.48 (2 H, m), 2.60 (3 H, s), 3.81 (1 H, quind, J=8.62, 8.62, 8.62, 8.62, 1.10 Hz), 4.84 (1 H, quip, J=6.98 Hz), 7.35 (1 H, dd, J=12.05, 1.09 Hz), 7.73 (1 H, d, J=1.37 Hz), 7.96 (1 H, d, J=2.19 Hz), 9.26 (1 H, s), 12.20 (1 H, br s); ESIMS found for C_{2I}F1₂₂FN₅m/z 364.2 (M+1).

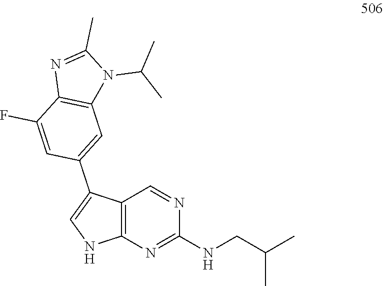 [SMILES: Cc1nc2c(F)cc(-c3c[nH]c4nc(NCC(C)C)ncc34)cc2n1C(C)C]

5-(4-Fluoro-14 sopropyl-2-methyl-1H-benzo [d] imidazol-6-yl)-N-i sobutyl-7H-pyrrolo [2,3-d]pyrimidin-2-amine 506.

Beige solid (20 mg, 0.053 mmol, 56.2% yield). ¹H NMR (499 MHz, DMSO-d₆) δppm 0.91 (6 H, d, J=6.57 Hz), 1.60 (6 H, d, J=6.84 Hz), 1.87-1.98 (1 H, m), 2.58 (3 H, s), 3.13 (2 H, t, J=6.43 Hz), 4.82 (1 H, quip, J=6.91 Hz), 6.76-6.85 (1 H, m), 7.25 (1 H, dd, J=12.05, 1.09 Hz), 7.48 (1 H, d, J=2.46 Hz), 7.61(1 H, d, J=1.37 Hz), 8.82 (1 H, s), 11.43 (1 H, br s); ESIMS found for C2,H₂₅FN6 m/z 381.1 (M+1).

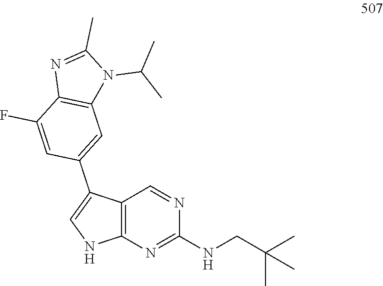 [SMILES: Cc1nc2c(F)cc(-c3c[nH]c4nc(NCC(C)(C)C)ncc34)cc2n1C(C)C]

5-(4-Fluoro-1-isopropyl-2-methyl-1H-benzo[d]imidazol-6-yl)-N-neopentyl-7H-pyrrolo[2,3-d]pyrimidin-2-amine 507.

Pink solid (25 mg, 0.063 mmol, 69.5% yield). ¹H NMR (499 MHz, DMSO-d₆) δppm 0.93 (9 H, s), 1.60 (6 H, d, J=7.12 Hz), 2.58 (3 H, s), 3.23 (2 H, d, J=6.30 Hz), 4.82 (1 H, quin, J=6.91 Hz), 6.65 (1 H, brt, J=6.16 Hz), 7.25 (1 H, dd, J=12.05, 1.10 Hz), 7.48 (1 H, d, J=2.19 Hz), 7.61 (1 H, d, J=1.37 Hz), 8.81 (1 H, s), 11.42 (1 H, br s); ESIMS found for C₂₂H₂₇FN₆ m/z 395.3 (M+1).

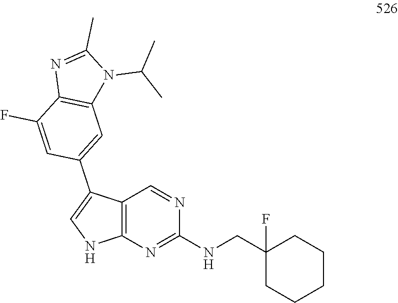 [SMILES: Cc1nc2c(F)cc(-c3c[nH]c4nc(NCC5(F)CCCCC5)ncc34)cc2n1C(C)C]

5-(4-Fluoro-1-isopropyl-2-methyl-1H-benzo[d]imidazol-6-yl)-N-((1-fluorocyclohexyl)methyl)-7H-pyrrolo[2,3-d]pyrimidin-2-amine 526.

Beige solid (20 mg, 0.046 mmol, 45.1% yield). ¹I-1 NMR (499 MHz, DMSO-d₆) δppm 1.20-1.30 (1 H, m), 1.45-1.57 (7 H, m), 1.60 (6 H, d, J=6.84 Hz), 1.73-1.82 (2 H, m), 2.58 (3 H, s), 3.60 (2 H, dd, J=20.60, 6.30 Hz), 4.82 (1 H, quin, J=6.91 Hz), 6.75 (1 H, brt, J=6.02 Hz), 7.26 (1 H, dd, J=12.05, 0.82 Hz), 7.51 (1 H, d, J=2.19 Hz), 7.62 (1 H, d, J=1.09 Hz), 8.84 (1 H, s), 11.47 (1 H, br s); ESIMS found for C₂₄H₂₈F₂N₆ m/z 439.2 (M+1).

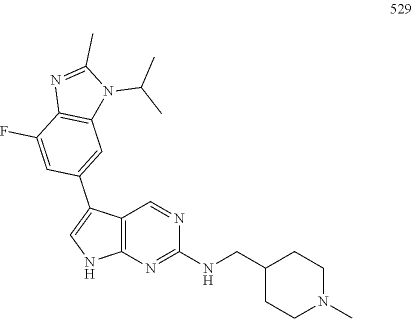 [SMILES: Cc1nc2c(F)cc(-c3c[nH]c4nc(NCC5CCN(C)CC5)ncc34)cc2n1C(C)C]

5-(4-Fluoro-1-isopropyl-2-methyl-1H-benzo[d]imidazol-6-yl)-N-((1-methylpiperidin-4-yl)methyl)-7H-pyrrolo[2,3-d]pyrimidin-2-a 529.

Beige solid (12 mg, 0.028 mmol, 27.1% yield). ¹I-1 NMR (499 MHz, DMSO-d₆) δppm 1.17 (2 H, qd, J=11.98, 3.57 Hz), 1.52-1.58 (1 H, m), 1.60 (6 H, d, J=6.86 Hz), 1.68 (2 H, br d, J=11.53 Hz), 1.78 (2 H, td, J=11.60, 2.06 Hz), 2.12 (3 H, s), 2.58 (3 H, s), 2.68-2.77 (2 H, m), 3.19 (2 H, t, J=6.45 Hz), 4.82 (1 H, quin, J=7.00 Hz), 6.79 (1 H, br t, J=5.63 Hz), 7.25 (1 H, dd, J=12.08, 0.82 Hz), 7.48 (1 H, d, J=1.92 Hz), 7.61 (1 H, d, J=1.10 Hz), 8.81 (1 H, s), 11.44 (1 H, br s); ESIMS found for C₂₄H₃₀FN₇ m/z 436.25 (M+1).

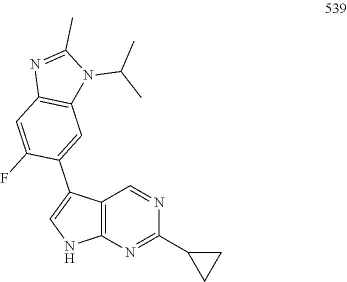 [SMILES: Cc1nc2cc(F)c(-c3c[nH]c4nc(C5CC5)ncc34)cc2n1C(C)C]

2—Cyclopropyl-5-(5-fluoro-1-i sopropyl-2-methyl-1H-benzo [d] imidazol-6-yl)-7H-pyrrolo [2,3-d]pyrimidine 539.

White solid (5 mg, 0.014 mmol, 7.1% yield). ¹H NMR (499 MHz, DMSO-d₆) δppm 0.99-1.04 (4 H, m), 1.59 (6 H, d, J=7.12 Hz), 2.23 (1 H, tt, J=7.73, 4.86 Hz), 2.58 (3 H, s), 4.80 (1 H, quill, J=6.98 Hz), 7.44 (1 H, d, J=11.50 Hz), 7.71 (1 H, s), 7.81 (1 H, d, J=6.57 Hz), 8.91 (1 H, d, J=2.19 Hz), 12.16 (1 H, br s); ESIMS found for C20H₂₀FN5 m/z 350.2 (M+1).

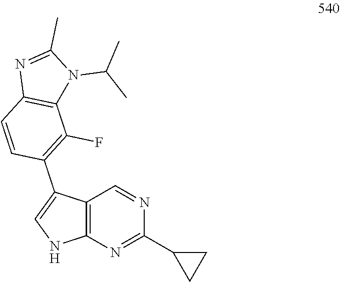 [SMILES: Cc1nc2ccc(-c3c[nH]c4nc(C5CC5)ncc34)c(F)c2n1C(C)C]

2—Cyclopropyl-5-(7-fluoro-1-isopropyl-2-methyl-1H-benzo [d] imidazol-6-yl)-7H-pyrrolo [2,3-d]pyrimidine 540.

Off-white solid (6 mg, 0.017 mmol, 16.5% yield). ¹1-1 NMR (499 MHz, DMSO-d₆) δppm 0.98-1.04 (4 H, m), 1.55 (6 H, dd, J=6.98, 1.51 Hz), 2.18-2.29 (1 H, m), 2.61 (3 H, s), 4.85 (1 H, br s), 7.43 (1 H, s), 7.44 (1 H, s), 7.69 (1 H, s), 8.93 (1 H, d, J=2.46 Hz), 12.16 (1 H, s); ESIMS found for C20H₂₀FN5 m/z 350.2 (M+1).

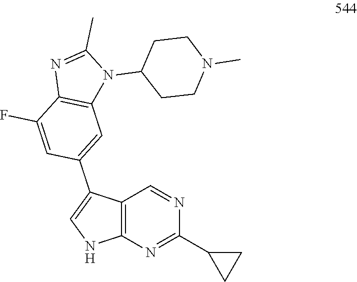 [SMILES: Cc1nc2c(F)cc(-c3c[nH]c4nc(C5CC5)ncc34)cc2n1C1CCN(C)CC1]

2—Cyclopropyl-5-(4-fluoro-2-methyl-1-(1-methylpiperidin-4-yl)-1H-benzo[d] imidazol-6-yl)-7H-pyrrolo[2,3-d]pyrimidine 544.

Off-white solid (18 mg, 0.045 mmol, 28.5% yield). ¹1-1 NMR (499 MHz, DMSO-d₆) δppm 0.99-1.07(4 H, m), 1.82-1.92 (2 H, m), 2.11-2.19 (2 H, m), 2.21-2.25 (1 H, m), 2.26 (3 H, s), 2.43 (2 H, qd, J=12 .27 , 3.42 Hz), 2.62 (3 H, s), 2.95 (2 H, br d, J=10.95 Hz), 4.30-4.41 (1 H, m), 7.34 (1 H, dd, J=11.77, 1.10 Hz), 7.65 (1 H, d, J=1.37 Hz), 7.90(1 H, s), 9.12(1 H, s), 12.12 (1 H, br s); ESIMS found for C23H₂₅FN6 m/z 405.2 (M+1).

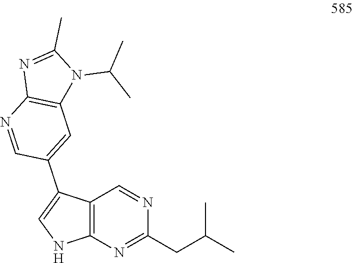 [SMILES: Cc1nc2ncc(-c3c[nH]c4nc(CC(C)C)ncc34)cc2n1C(C)C]

6-(2-Isobutyl-7H-pyrrolo [2,3-d] pyrimidin-5-yl)-1-i sopropyl-2-methyl-1H-imidazo[4,5-b]pyridine 585.

Off-white solid (58 mg, 0.167 mmol, 42.8% yield). ¹H NMR (499 MHz, DMSO-d₆) δppm 0.94 (6 H, d, J=6.57 Hz), 1.62 (6 H, d, J=6.84 Hz), 2.29 (1 H, dquin, J=13.52, 6.82, 6.82, 6.82, 6.82 Hz), 2.63 (3 H, s), 2.80 (2 H, d, J=7.12 Hz), 4.84 (1 H, spt, J=6.94 Hz), 8.00 (1 H, s), 8.30 (1 H, d, J=2.19 Hz), 8.72 (1 H, d, J=1.92 Hz), 9.24 (1 H, s), 12.21 (1 H, br s); ESIMS found for C20H₂₄N6 m/z 349.2 (M+1).

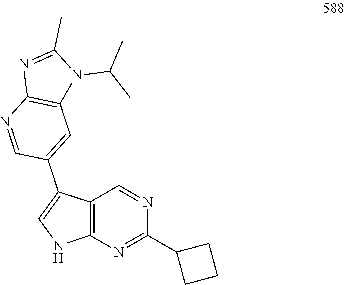 [SMILES: Cc1nc2ncc(-c3c[nH]c4nc(C5CCC5)ncc34)cc2n1C(C)C]

6-(2—Cyclobutyl-7H-pyrrolo[2,3-d]pyrimidin-5-yl)-1-isopropyl-2-methyl-1H-imidazo[4,5-b]pyridine 588.

Beige solid (10 mg, 0.029 mmol, 15.7% yield). ¹H NMR (499 MHz, DMSO-d₆) δppm 1.62 (6 H, d, J=6.84 Hz), 1.85-1.94 (1 H, m), 1.98-2.11 (1 H, m), 2.28-2.37 (2 H, m), 2.39-2.48 (2 H, m), 2.63 (3 H, s), 3.75-3.86 (1 H, m), 4.83 (1 H, quin, J=6.91 Hz), 8.00 (1 H, s), 8.27 (1 H, d, J=1.92 Hz), 8.71 (1 H, d, J=2.19 Hz), 9.23 (1 H, s); ESIMS found for C₂₀H₂₂N₆ m/z 347.15 (M+1).

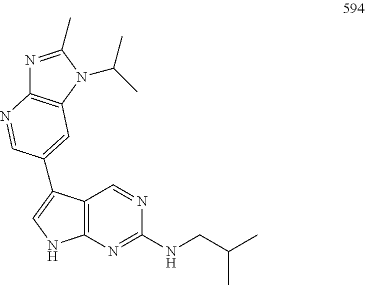 [SMILES: Cc1nc2ncc(-c3c[nH]c4nc(NCC(C)C)ncc34)cc2n1C(C)C]

N-Isobutyl-5-(1-isopropyl-2-methyl-1H-imidazo [4,5-b] pyridin-6-yl)-7H-pyrrolo [2,3-d] pyrimidin-2-amine 594.

Beige solid (10 mg, 0.028 mmol, 15.8% yield). ¹H NMR (499 MHz, DMSO-d₆) δppm 0.91 (6 H, d, J=6.84 Hz), 1.60 (6 H, d, J=7.12 Hz), 1.92 (1 H, dquin, J=13.59, 6.73, 6.73, 6.73, 6.73 Hz), 2.62 (3 H, s), 3.13 (2 H, t, J=6.57 Hz), 4.82 (1 H, quill, J=6.91 Hz), 6.82 (1 H, br t, J=4.79 Hz), 7.53 (1 H, s), 8.17 (1 H, d, J=1.92 Hz), 8.63 (1 H, d, J=1.92 Hz), 8.82 (1 H, s), 11.49 (1 H, br s); ESIMS found for C₂₀1⁻1₂₅1\1₇m/z 364.2 (M+1).

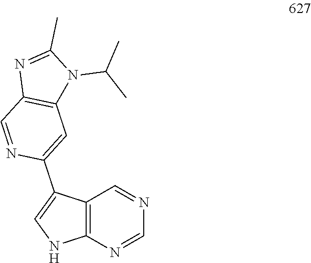 [SMILES: Cc1nc2cnc(-c3c[nH]c4ncncc34)cc2n1C(C)C]

1-Isopropyl-2-methyl-6-(7H-pyrrolo [2,3-d] pyrimidin-5-yl)-1H-imidazo [4,5-c]pyridine 627.

Beige solid (25 mg, 0.086 mmol, 63.6% yield). ¹H NMR (499 MHz, DMSO-d₆) δppm 1.63 (6 H, d, J=6.84 Hz), 2.61 (3 H, s), 4.80 (1 H, quill, J=6.98 Hz), 8.14 (1 H, d, J=1.10 Hz), 8.42 (1 H, s), 8.79 (1 H, s), 8.85 (1 H, d, J=1.10 Hz), 9.77 (1 H, s), 12.34 (1 H, br s); ESIMS found for C₁₆H₁₆N6 m/z 293.1 (M+1).

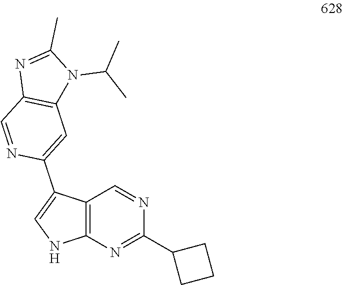 [SMILES: Cc1nc2cnc(-c3c[nH]c4nc(C5CCC5)ncc34)cc2n1C(C)C]

6-(2—Cyclobutyl-7H-pyrrolo [2,3-d]pyrimidin-5-yl)-1-isopropyl-2-methyl-1H-imidazo[4,5-c]pyridine 628.

Beige solid (15 mg, 0.043 mmol, 21.7% yield). ¹H NMR (499 MHz, DMSO-d₆) δppm 1.63 (6 H, d, J=6.84 Hz), 1.84-1.95 (1 H, m), 1.97-2.12 (1 H, m), 2.26-2.37 (2 H, m), 2.38-2.48 (2 H, m), 2.61 (3 H, s), 3.75-3.86 (1 H, m), 4.79 (1 H, J=6.91 Hz), 8.09 (1 H, d, J=1.10 Hz), 8.30 (1 H, s), 8.84 (1 H, d, J=0.82 Hz), 9.67 (1 H, s), 12.16 (1 H, br s); ESIMS found for C₂₀H₂₂N₆ m/z 347.2 (M+1).

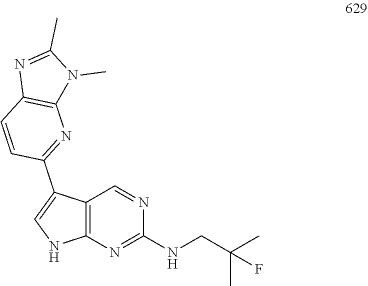 [SMILES: Cc1nc2ccc(-c3c[nH]c4nc(NCC(C)(C)F)ncc34)nc2n1C]

5-(2,3-Dimethyl-3H-imidazo [4,5-1)] pyridin-5-yl)-N-(2-fluoro-2-methylpropyl)-7H-pyrrolo [2,3-d] pyrimidin-2-amine 629.

White solid (26 mg, 0.074 mmol, 32.6% yield). ¹H NMR (499 MHz, DMSO-d₆) δppm 1.35 (6 H, d, J=21.40 Hz), 2.56 (3 H, s), 3.60 (2 H, dd, J=19.16, 6.57 Hz), 3.82 (3 H, s), 6.80 (1 H, br t, J=6.30 Hz), 7.68 (1 H, d, J=8.21 Hz), 7.80 (1 H, d, J=2.19 Hz), 7.85 (1 H, d, J=8.21 Hz), 9.44 (1 H, s), 11.51 (1 H, br s); ESIMS found for C₁₈H₂₀FN₇ m/z 354.2 (M+1).

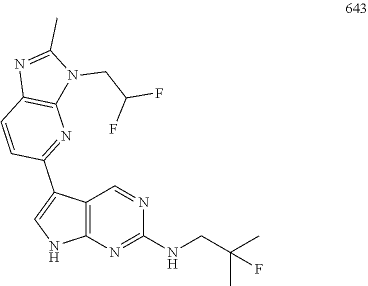 [SMILES: Cc1nc2ccc(-c3c[nH]c4nc(NCC(C)(C)F)ncc34)nc2n1CC(F)F]

5-(3-(2,2-Difluoroethyl)-2-methyl-3H-imidazo [4,5-b]pyridin-5-A-N-(2-fluoro-2-methylpropyl)-7H-pyrrolo [2,3-d]pyrimidin-2-amine 643.

Yellow white solid (6 mg, 0.015 mmol, 3.4% yield). ¹H NMR (499 MHz, DMSO-d₆) δppm 1.35 (6 H, d, J=21.40 Hz), 2.60 (3 H, s), 3.61 (2 H, br dd, J=19.44, 6.30 Hz), 4.83 (2 H, td, J=16.02, 3.01 Hz), 6.55 (1 H, tt, J=54.90, 3.10 Hz), 6.77 (1 H, br t, J=6.30 Hz), 7.72 (1 H, d, J=8.76 Hz), 7.82 (1 H, d, J=2.74 Hz), 7.90 (1 H, d, J=8.21 Hz), 8.19 (1 H, s), 9.40 (1 H, s), 11.53 (1 H, br s); ESIMS found for C₁₉H₂₀F₃N₇ m/z 404.2 (M+1).

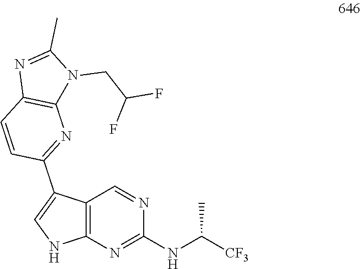 [SMILES: Cc1nc2ccc(-c3c[nH]c4nc(N[C@H](C)C(F)(F)F)ncc34)nc2n1CC(F)F]

(R)-5-(3-(2,2-Difluoroethyl)-2-methyl-3H-imidazo[4,5-b]pyridin-5-yl)-N-(1,1,1-trifluoropropan-2-yl)-7H-pyrrolo[2,3-d]pyrimidin-2-amine 646.

Off-white solid (25 mg, 0.059 mmol, 27.7% yield). ¹H NMR (499 MHz, DMSO-d₆) δppm 1.36 (3 H, d, J=7.12 Hz), 2.60 (3 H, s), 4.78-4.90 (2 H, m), 4.93-5.05 (1 H, m), 6.55 (1 H, tt, J=54.50, 3.30 Hz), 7.28 (1 H, br d, J=9.31 Hz), 7.74 (1 H, d, J=8.76 Hz), 7.89 (1 H, s), 7.91 (1 H, d, J=8.76 Hz), 9.45 (1 H, s), 11.66 (1 H, br s); ESIMS found for CisH₁₆F₅N₇ m/z 426.1 (M+1).

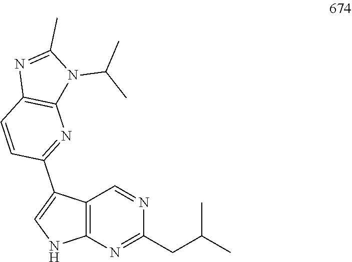 [SMILES: Cc1nc2ccc(-c3c[nH]c4nc(CC(C)C)ncc34)nc2n1C(C)C]

5-(2-Isobutyl-7H-pyrrolo [2,3-d] pyrimidin-5-yl)-3-i sopropyl-2-methyl-3H-imidazo[4,5-b]pyridine 674.

Off-white solid (26 mg, 0.075 mmol, 33.4% yield). ¹H NMR (499 MHz, DMSO-d₆) δppm 0.94 (6 H, d, J=6.57 Hz), 1.76 (6 H, d, J=6.84 Hz), 2.29 (1 H, dquin, J=13.62, 6.86, 6.86, 6.86, 6.86 Hz), 2.61 (3 H, s), 2.80 (2 H, d, J=7.12 Hz), 4.87 (1 H, spt, J=6.80 Hz), 7.77 (1 H, d, J=8.49 Hz), 7.89 (1 H, d, J=8.21 Hz), 8.23 (1 H, s), 9.75 (1 H, s), 12.19 (1 H, br s); ESIMS found for C20H₂₄N6 m/z 349.2 (M+1).

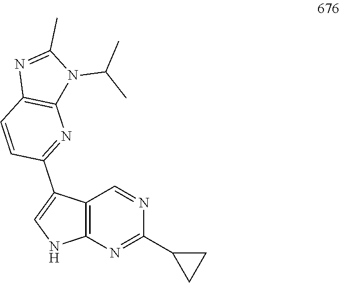 [SMILES: Cc1nc2ccc(-c3c[nH]c4nc(C5CC5)ncc34)nc2n1C(C)C]

5-(2—Cyclopropyl-7H-pyrrolo[2,3-d]pyrimidin-5-yl)-3-isopropyl-2-methyl-3H-imidazo [4,5-b]pyridine 676.

Off-white solid (5 mg, 0.015 mmol, 5.8% yield). ¹H NMR (499 MHz, DMSO-d₆) δppm 0.97-1.08 (4 H, m), 1.75 (6 H, d, J=6.84 Hz), 2.23 (1 H, tt, J=7.94, 4.93 Hz), 2.61 (3 H, s), 4.86 (1 H, spt, J=6.80 Hz), 7.75 (1 H, d, J=8.21 Hz), 7.88 (1 H, d, J=8.21 Hz), 8.17 (1 H, s), 9.66 (1 H, s), 12.12 (1 H, br s); ESIMS found for C₁₉H₂₀N₆ m/z 333.2 (M+1).

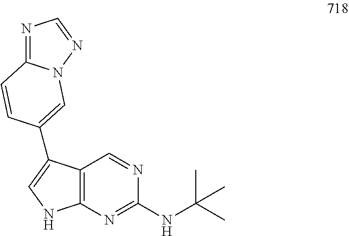 [SMILES: CC(C)(C)Nc1ncc2c(-c3ccc4ncnn4c3)c[nH]c2n1]

5-([1,2,4]Triazolo[1,5-a]pyridin-6-yl)-N-(tert-butyl)-7H-pyrrolo[2,3-d] pyrimidin-2-amine 718.

Beige solid (15 mg, 0.049 mmol, 45.1% yield). ¹H NMR (499 MHz, DMSO-d₆) δppm 0.93 (9 H, s), 3.22 (2 H, d, J=6.30 Hz), 6.77 (1 H, br s), 7.63 (1 H, d, J=1.92 Hz), 7.87 (1 H, dd, J=9.31, 0.82 Hz), 8.06 (1 H, dd, J=9.31, 1.64 Hz), 8.48 (1 H, s), 8.94 (1 H, s), 9.19 (1 H, dd, J=1.64, 0.82 Hz), 11.58 (1 H, br s); ESIMS found for C₁₆H₁₇N₇ m/z 308.15 (M+1).

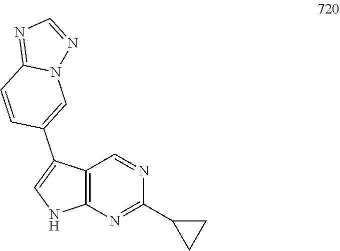 [SMILES: c1nc2ccc(-c3c[nH]c4nc(C5CC5)ncc34)cn2n1]

6-(2—Cyclopropyl-7H-pyrrolo [2,3-d] pyrimidin-5-yl)41,2,41triazolo [1,5-a] pyridine 720.

Tan solid (20 mg, 0.072 mmol, 41.5% yield). ¹H NMR (499 MHz, DMSO-d₆) δppm 1.00-1.06 (4 H, m), 2.21-2.29 (1 H, m), 7.89-7.94 (1 H, m), 8.03 (1 H, d, J=2.46 Hz), 8.12 (1 H, dd, J=9.17, 1.78 Hz), 8.51 (1 H, s), 9.27 (1 H, s), 9.30 (1 H, dd, J=1.64, 0.82 Hz), 12.25 (1 H, br s); ESIMS found for CisH₁₂N₆ m/z 277.1 (M+1).

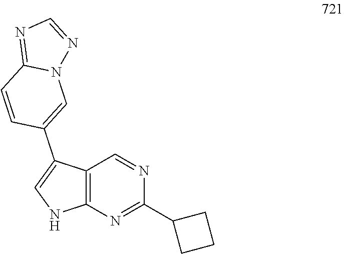 [SMILES: c1nc2ccc(-c3c[nH]c4nc(C5CCC5)ncc34)cn2n1]

6-(2—Cyclobutyl-7H-pyrrolo [2,3-d]pyrimidin-5-yl)-[1,2,4]triazolo [1,5-a] pyridine 721.

Beige solid (5 mg, 0.017 mmol, 10.3% yield). ¹H NMR (499 MHz, DMSO-d₆) δppm 1.85-1.95 (1 H, m), 1.99-2.11 (1 H, m), 2.29-2.38 (2 H, m), 2.39-2.48 (2 H, m), 3.77-3.88 (1 H, m), 7.93 (1 H, d, J=9.31 Hz), 8.10 (1 H, s), 8.14 (1 H, dd, J=9.17, 1.78 Hz), 8.51 (1 H, s), 9.32 (1 H, d, J=0.82 Hz), 9.37 (1 H, s), 12.36 (1 H, br s); ESIMS found for C₁₆H₁₄N6 m/z 291.15 (M+1).

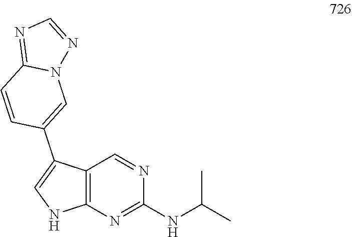 [SMILES: CC(C)Nc1ncc2c(-c3ccc4ncnn4c3)c[nH]c2n1]

5-([1,2,4] Triazolo [1,5-a] pyridin-6-yl)-N-i sopropyl-7H-pyrrolo [2,3-d] pyrimidin-2-amine 726.

Light pink solid (15 mg, 0.051 mmol, 39.5% yield). ¹H NMR (499 MHz, DMSO-d₆) δppm 1.18 (6 H, d, J=6.30 Hz), 4.01-4.13 (1 H, m), 6.65 (1 H, br d, J=7.67 Hz), 7.64 (1 H, d, J=2.19 Hz), 7.87 (1 H, dd, J=9.31, 0.82 Hz), 8.06 (1 H, dd, J=9.17, 1.78 Hz), 8.48 (1 H, s), 8.95 (1 H, s), 9.19 (1 H, dd, J=1.64, 0.82 Hz), 11.62 (1 H, br s); ESIMS found for CisH₁₅N7 m/z 294.1 (M+1).

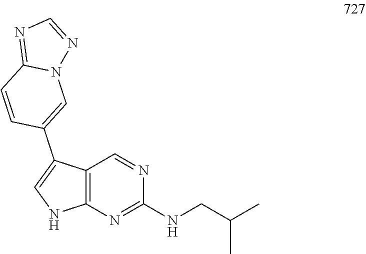 [SMILES: CC(C)CNc1ncc2c(-c3ccc4ncnn4c3)c[nH]c2n1]

5-([1,2,4] Triazolo [1,5-a] pyridin-6-yl)-N-i sobutyl-7H-pyrrolo [2,3-d] pyrimidin-2-amine 727.

Beige solid (23 mg, 0.075 mmol, 78.5% yield). ¹H NMR (499 MHz, DMSO-d₆) δppm 0.91 (6 H, d, J=6.84 Hz), 1.91 (1 H, dquin, J=13.52, 6.75, 6.75, 6.75, 6.75 Hz), 3.13 (2 H, t, J=6.43 Hz), 6.91 (1 H, br s), 7.63 (1 H, d, J=2.46 Hz), 7.87 (1 H, dd, J=9.31, 0.82 Hz), 8.06 (1 H, dd, J=9.31, 1.64 Hz), 8.48 (1 H, s), 8.94 (1 H, s), 9.18 (1 H, dd, J=1.64, 0.82 Hz), 11.60 (1 H, br s); ESIMS found for C₁₆H₁₇N₇ m/z 308.15 (M+1).

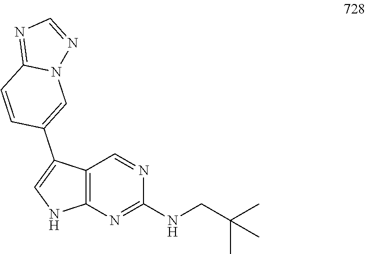 [SMILES: CC(C)(C)CNc1ncc2c(-c3ccc4ncnn4c3)c[nH]c2n1]

5-([1,2,4] Triazolo [1,5-a] pyridin-6-yl)-N-neopentyl-7H-pyrrolo [2,3-d] pyrimidin-2-amine 728.

Pink solid (30 mg, 0.093 mmol, 59.2% yield). ¹H NMR (499 MHz, DMSO-d₆) δppm 0.93 (9 H, s), 3.22 (2 H, d, J=6.30 Hz), 6.77 (1 H, br s), 7.63 (1 H, d, J=1.92 Hz), 7.87 (1 H, dd, J=9.31, 0.82 Hz), 8.06 (1 H, dd, J=9.31, 1.64 Hz), 8.48 (1 H, s), 8.94 (1 H, s), 9.19 (1 H, dd, J=1.64, 0.82 Hz), 11.58 (1 H, br s); ESIMS found for C₁₇H₁₉N₇ m/z 322.2 (M+1).

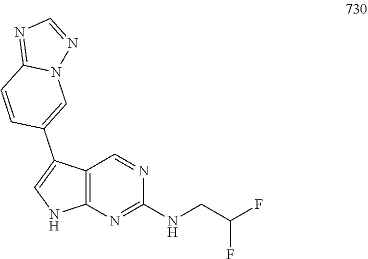 [SMILES: FC(F)CNc1ncc2c(-c3ccc4ncnn4c3)c[nH]c2n1]

5-([1,2,4] Triazolo [1,5-a] pyridin-6-yl)-N-(2,2-difluoroethyl)-7H-pyrrolo [2,3-d]pyrimidin-2-amine 730.

Beige solid (3 mg, 0.010 mmol, 31.9% yield). ¹H NMR (499 MHz, DMSO-d₆) δppm 3.66-3.79 (2 H, m), 6.14 (1 H, tt, J=56.80, 4.40 Hz), 7.24 (1 H, br t, J=5.34 Hz), 7.72 (1 H, d, J=2.19 Hz), 7.88 (1 H, d, J=9.31 Hz), 8.07 (1 H, dd, J=9.17, 1.78 Hz), 8.49 (1 H, s), 9.02 (1 H, s), 9.22 (1 H, s), 11.77 (1 H, br s); ESIMS found for C₁₄H₁₁F₂N₇ m/z 316.3 (M+1).

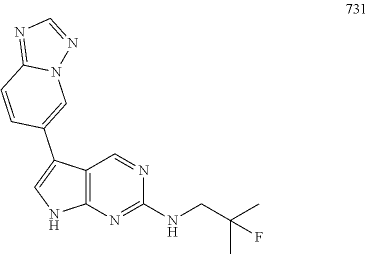 [SMILES: CC(C)(F)CNc1ncc2c(-c3ccc4ncnn4c3)c[nH]c2n1]

5-([1,2,4] Triazolo [1,5-a] pyridin-6-yl)-N-(2-fluoro-2-methylpropyl)-7H-pyrrolo [2,3-d]pyrimidin-2-amine 731.

White solid (35 mg, 0.108 mmol, 24.5% yield). ¹H NMR (499 MHz, DMSO-d₆) δppm 1.35 (6 H, d, J=21.40 Hz), 3.59 (2 H, dd, J=19.03, 6.43 Hz), 6.93 (1 H, br t, J=5.61 Hz), 7.67 (1 H, d, J=1.64 Hz), 7.88 (1 H, dd, J=9.31, 0.82 Hz), 8.06 (1 H, dd, J=9.17, 1.78 Hz), 8.48 (1 H, s), 8.97 (1 H, s), 9.20 (1 H, dd, J=1.64, 0.82 Hz), 11.65 (1 H, br s); ESIMS found for C₁₆H₁₆FN₇ m/z 326.2 (M+1).

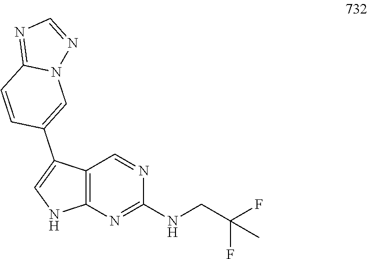 [SMILES: CC(F)(F)CNc1ncc2c(-c3ccc4ncnn4c3)c[nH]c2n1]

5-([1,2,4]Triazolo[1,5-a]pyridin-6-yl)-N-(2,2-difluoropropyl)-7H-pyrrolo [2,3-d]pyrimidin-2-amine 732.

Beige solid (3 mg, 0.009 mmol, 18.4% yield). ¹H NMR (499 MHz, DMSO-d₆) δppm 1.63 (3 H, t, J=19.03 Hz), 3.83 (2 H, td, J=13.76, 6.71 Hz), 7.22 (1 H, br t, J=5.89 Hz), 7.71 (1 H, d, J=2.19 Hz), 7.85-7.92 (1 H, m), 8.07 (1 H, dd, J=9.31, 1.92 Hz), 8.49 (1 H, s), 9.01 (1 H, s), 9.21 (1 H, d, J=0.82 Hz), 11.72 (1 H, br s); ESIMS found for CisH₁₃F₂N₇ m/z 330.1 (M+1).

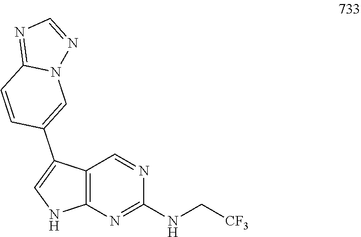 [SMILES: FC(F)(F)CNc1ncc2c(-c3ccc4ncnn4c3)c[nH]c2n1]

5-([1,2,4]Triazolo[1,5-a]pyridin-6-yl)-N-(2,2,2-trifluoroethyl)-7H-pyrrolo [2,3-d]pyrimidin-2-amine 733.

White solid (7.12 mg, 0.021 mmol). ¹H NMR (400 MHz, DMSO-d₆) δppm 4.11-4.26 (2 H, m), 7.49 (1 H, br t, J=6.60 Hz), 7.74 (1 H, d, J=2.32 Hz), 7.88 (1 H, d, J=9.41 Hz), 8.07 (1 H, dd, J=9.29, 1.83 Hz), 8.49 (1 H, s), 9.04 (1 H, s), 9.23 (1 H, s), 11.82 (1 H, br s); ESIMS found for CHHI0F3N7 m/z 334.1 (M+1).

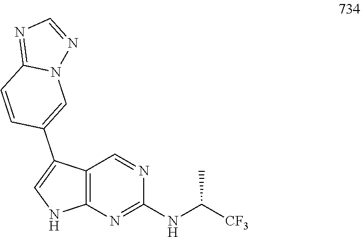 [SMILES: C[C@@H](Nc1ncc2c(-c3ccc4ncnn4c3)c[nH]c2n1)C(F)(F)F]

(R)-5-([1,2,4] Triazolo [1,5-a] pyridin-6-yl)-N-(1,1,1-trifluoropropan-2-yl)-7H-pyrrolo [2,3-d]pyrimidin-2-amine 734.

White solid (31.23 mg, 0.090 mmol). ¹H NMR (400 MHz, DMSO-d₆) δppm 1.36 (3 H, d, J=7.00 Hz), 4.88-5.03 (1 H, m), 7.44 (1 H, br d, J=9.01 Hz), 7.74 (1 H, s), 7.89 (1 H, d, J=9.26 Hz), 8.08 (1 H, dd, J=9.26, 1.63 Hz), 8.49 (1 H, s), 9.03 (1 H, s), 9.23 (1 H, s), 11.80 (1 H, br s); ESIMS found for CisH₁₂F₃N₇ m/z 348.0 (M+1).

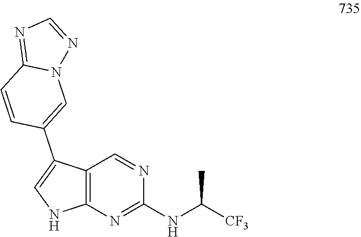 [SMILES: C[C@H](Nc1ncc2c(-c3ccc4ncnn4c3)c[nH]c2n1)C(F)(F)F]

(S)-5-([1,2,4]Triazolo[1,5-a]pyridin-6-yl)-N-(1,1,1-trifluoropropan-2-yl)-7H-pyrrolo[2,3-d]pyrimidin-2-amine 735.

White solid (45.25 mg, 0.130 mmol). ¹H NMR (400 MHz, DMSO-d₆) δppm 1.36 (3 H, d, J=7.13 Hz), 4.88-5.02 (1 H, m), 7.38 (1 H, br d, J=8.88 Hz), 7.74 (1 H, s), 7.88 (1 H, d, J=9.26 Hz), 8.07 (1 H, dd, J=9.32, 1.69 Hz), 8.48 (1 H, s), 9.02 (1 H, s), 9.21 (1 H, s), 11.70 (1 H, br s); ESIMS found for CisH₁₂F₃N₇ m/z 348.1 (M+1).

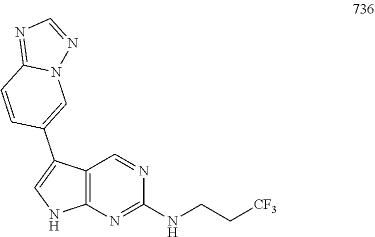 [SMILES: FC(F)(F)CCNc1ncc2c(-c3ccc4ncnn4c3)c[nH]c2n1]

5-([1,2,4] Triazolo [1,5-a] pyridin-6-yl)-N-(3,3,3-trifluoropropyl)-7H-pyrrolo [2,3-d]pyrimidin-2-amine 736.

White solid (2 mg, 0.006 mmol, 13.1% yield). ¹H NMR (499 MHz, DMSO-d₆) δppm 2.55-2.67 (2 H, m), 3.52-3.60 (2 H, m), 7.02 (1 H, br t, J=4.52 Hz), 7.69 (1 H, d, J=2.46 Hz), 7.88 (1 H, dd, J=9.31, 0.82 Hz), 8.07 (1 H, dd, J=9.17, 1.78 Hz), 8.48 (1 H, s), 9.00 (1 H, s), 9.21 (1 H, dd, J=1.64, 0.82 Hz), 11.70 (1 H, br s); ESIMS found for CisH₁₂F₃N₇ m/z 348.1 (M+1).

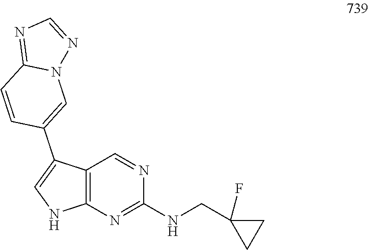 [SMILES: FC1(CNc2ncc3c(-c4ccc5ncnn5c4)c[nH]c3n2)CC1]

5-([1,2,4] Triazolo [1,5-a] pyridin-6-yl)-N-((1-fluorocyclopropyl)methyl)-7H-pyrrolo [2,3-d]pyrimidin-2-amine 739.

White solid (3 mg, 0.009 mmol, 9.6% yield). ¹H NMR (500 MHz, DMSO-d₆) δppm 0.78-0.86 (2 H, m), 0.92-1.02 (2 H, m), 3.82 (2 H, dd, J=19.21, 6.31 Hz), 7.09 (1 H, br t, J=5.49 Hz), 7.67 (1 H, s), 7.88 (1 H, dd, J=9.33, 0.82 Hz), 8.06 (1 H, dd, J=9.33, 1.92 Hz), 8.48 (1 H, s), 8.97 (1 H, s), 9.20 (1 H, dd, J=1.92, 0.82 Hz), 11.65 (1 H, br s); ESIMS found for C₁₆H₁₄FN₇ m/z 324.1 (M+1).

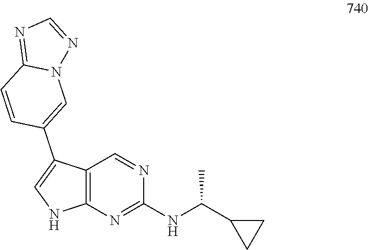 [SMILES: C[C@@H](Nc1ncc2c(-c3ccc4ncnn4c3)c[nH]c2n1)C1CC1]

(R)-5-([1,2,4] Triazolo [1,5-a] pyridin-6-yl)-N-(1-cyclopropylethyl)-7H-pyrrolo [2,3-d]pyrimidin-2-amine 740.

White solid (28.75 mg, 0.090 mmol). ¹H NMR (400 MHz, DMSO-d₆) δppm 0.10-0.21 (1 H, m), 0.29-0.47 (3 H, m), 0.93-1.07 (1 H, m), 1.21 (3 H, d, J=6.48 Hz), 3.57 (1 H, dt, J=14.61, 7.24 Hz), 6.71 (1 H, br d, J=8.44 Hz), 7.62 (1 H, d, J=2.08 Hz), 7.87 (1 H, d, J=9.29 Hz), 8.05 (1 H, dd, J=9.29, 1.59 Hz), 8.48 (1 H, s), 8.93 (1 H, s), 9.18 (1 H, s), 11.57 (1 H, br s); ESIMS found for C₁₇H₁₇N7 m/z 320.3 (M+1).

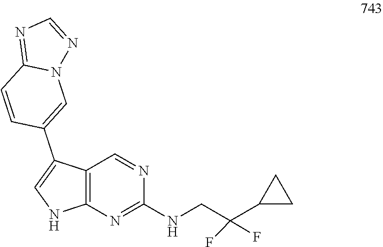 [SMILES: FC(F)(CNc1ncc2c(-c3ccc4ncnn4c3)c[nH]c2n1)C1CC1]

5-([1,2,4] Triazolo [1,5-a] pyridin-6-yl)-N-(2-cyclopropyl-2,2-difluoroethyl)-7H-pyrrolo[2,3-d]pyrimidin-2-amine 743.

White solid (2 mg, 0.006 mmol, 9.0% yield). ¹H NMR (499 MHz, DMSO-d₆) δppm 0.51-0.59 (4 H, m), 1.40-1.50 (1 H, m), 3.93 (2 H, td, J=13.83, 6.84 Hz), 7.10-7.19 (1 H, m), 7.70 (1 H, s), 7.88 (1 H, d, J=9.31 Hz), 8.07 (1 H, dd, J=9.17, 1.78 Hz), 8.49 (1 H, s), 9.00 (1 H, s), 9.21 (1 H, s), 11.72 (1 H, br s); ESIMS found for C₁₇H₁₅F₂N₇ m/z 356.1 (M+1).

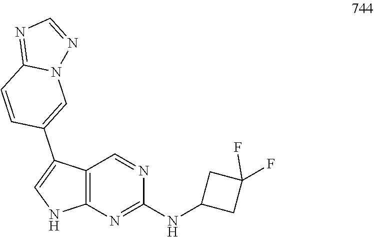 [SMILES: FC1(F)CC(Nc2ncc3c(-c4ccc5ncnn5c4)c[nH]c3n2)C1]

5-([1,2,4] Triazolo [1,5-a] pyridin-6-yl)-N-(3,3-difluorocyclobutyl)-7H-pyrrolo [2,3-d]pyrimidin-2-amine 744.

Light brown solid (9 mg, 0.026 mmol, 10.3% yield). ¹1-1 NMR (499 MHz, DMSO-d₆) δppm 2.59-2.71 (2 H, m), 2.91-3.01 (2 H, m), 4.20-4.31 (1 H, m), 7.41 (1 H, br d, J=6.02 Hz), 7.70 (1 H, d, J=2.19 Hz), 7.88 (1 H, d, J=9.03 Hz), 8.07 (1 H, dd, J=9.31, 1.92 Hz), 8.49 (1 H, s), 9.01 (1 H, s), 9.21 (1 H, s), 11.73 (1 H, br s); ESIMS found for C₁₆K₃F₂N₇ m/z 342.1 (M+1).

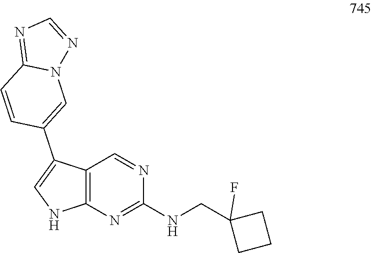 [SMILES: FC1(CNc2ncc3c(-c4ccc5ncnn5c4)c[nH]c3n2)CCC1]

5-([1,2,4]Triazolo[1,5-a]pyridin-6-yl)-N-((1-fluorocyclobutyl)methyl)-7H-pyrrolo [2,3-d]pyrimidin-2-amine 745.

White solid (28.51 mg, 0.085 mmol). ¹H NMR (400 MHz, DMSO-d₆) δppm 1.47-1.61 (1 H, m), 1.69-1.85 (1 H, m), 2.08-2.33 (4 H, m), 3.67-3.80 (2 H, m), 6.97 (1 H, br s), 7.67 (1 H, d, J=1.71 Hz), 7.87 (1 H, d, J=9.29 Hz), 8.06 (1 H, dd, J=9.23, 1.65 Hz), 8.48 (1 H, s), 8.97 (1 H, s), 9.20 (1 H, s), 11.66 (1 H, br s); ESIMS found for C₁₇H₁₆FN₇ m/z 338.0 (M+1).

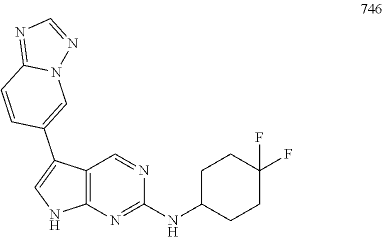 [SMILES: FC1(F)CCC(Nc2ncc3c(-c4ccc5ncnn5c4)c[nH]c3n2)CC1]

5-([1,2,4] Triazolo [1,5-a] pyridin-6-yl)-N-(4,4-difluorocyclohexyl)-7H-pyrrolo [2,3-d] pyrimidin-2-amine 746.

Off-white solid (19 mg, 0.051 mmol, 31.0% yield). ¹H NMR (499 MHz, DMSO-d₆) δppm 1.57-1.69 (2 H, m), 1.85-2.02 (4 H, m), 2.04-2.16 (2 H, m), 3.90-4.01 (1 H, m), 6.92 (1 H, br d, J=7.39 Hz), 7.65 (1 H, s), 7.87 (1 H, dd, J=9.17, 0.96 Hz), 8.06 (1 H, dd, J=9.17, 1.78 Hz), 8.48 (1 H, s), 8.97 (1 H, s), 9.20 (1 H, dd, J=1.78, 0.96 Hz), 11.66 (1 H, br s); ESIMS found for C₁₈H₁₇F2N7 m/z 370.15 (M+1).

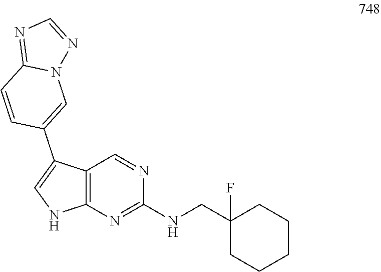 [SMILES: FC1(CNc2ncc3c(-c4ccc5ncnn5c4)c[nH]c3n2)CCCCC1]

5-([1,2,4]Triazolo[1,5-a]pyridin-6-yl)-N-((1-fluorocyclohexyl)methyl)-7H-pyrrolo [2,3-d]pyrimidin-2-amine 748.

Beige solid (20 mg, 0.055 mmol, 47.4% yield). ¹H NMR (499 MHz, DMSO-d₆) δppm 1.20-1.31 (1 H, m), 1.44-1.62 (7 H, m), 1.71-1.82 (2 H, m), 3.59 (2 H, dd, J=20.30, 6.60 Hz), 6.85 (1 H, br s), 7.66 (1 H, s), 7.83-7.91 (1 H, m), 8.06 (1 H, dd, J=9.17, 1.78 Hz), 8.48 (1 H, s), 8.96 (1 H, s), 9.19 (1 H, d, J=0.82 Hz), 11.63 (1 H, br s); ESIMS found for C₁₉H₂₀FN₇ m/z 366.2 (M+1).

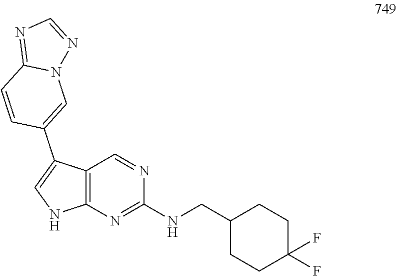 [SMILES: FC1(F)CCC(CNc2ncc3c(-c4ccc5ncnn5c4)c[nH]c3n2)CC1]

5-([1,2,4]Triazolo[1,5-a]pyridin-6-yl)-N-((4,4-difluorocyclohexyl)methyl)-7H-pyrrolo[2,3-d]pyrimidin-2-amine 749.

Beige solid (8 mg, 0.021 mmol, 18.7% yield). ¹H NMR (499 MHz, DMSO-d₆) δppm 1.16-1.29 (2 H, m), 1.67-1.89 (6 H, m), 1.95-2.08 (2 H, m), 3.23 (2 H, t, J=6.30 Hz), 7.00 (1 H, br s), 7.64 (1 H, s), 7.87 (1 H, dd, J=9.31, 0.82 Hz), 8.06 (1 H, dd, J=9.31, 1.92 Hz), 8.48 (1 H, s), 8.95 (1 H, s), 9.18 (1 H, dd, J=1.64, 0.82 Hz), 11.62 (1 H, br s); ESIMS found for C₁₉F1₁₉F₂N₇ m/z 384.2 (M+1).

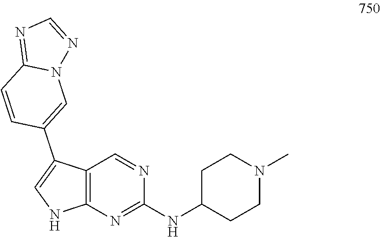 [SMILES: CN1CCC(Nc2ncc3c(-c4ccc5ncnn5c4)c[nH]c3n2)CC1]

5-([1,2,4] Triazolo [1,5-a] pyridin-6-yl)-N-(1-methylpiperidin-4-yl)-7H-pyrrolo [2,3-d]pyrimidin-2-amine 750.

White solid (5 mg, 0.014 mmol, 18.0% yield). ¹H NMR (499 MHz, DMSO-d₆) δppm 1.53 (2 H, qd, J=11.71, 3.57 Hz), 1.83-1.91 (2 H, m), 1.92-1.98 (2 H, m), 2.17 (3 H, s), 2.75 (2 H, br d, J=11.53 Hz), 3.63-3.77 (1 H, m), 6.72 (1 H, br d, J=6.86 Hz), 7.63 (1 H, s), 7.87 (1 H, dd, J=9.33, 0.82 Hz), 8.05 (1 H, dd, J=9.19, 1.78 Hz), 8.48 (1 H, s), 8.95 (1 H, s), 9.18 (1 H, dd, J=1.65, 0.82 Hz), 11.65 (1 H, br s); ESIMS found for Cₗaim⁻Ns m/z 349.1 (M+1).

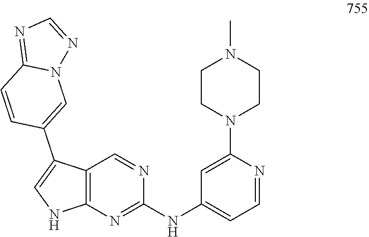 [SMILES: CN1CCN(c2cc(Nc3ncc4c(-c5ccc6ncnn6c5)c[nH]c4n3)ccn2)CC1]

5-([1,2,4]Triazolo[1,5-a]pyridin-6-yl)-N-(2-(4-methylpiperazin-1-yl)pyridin-4-yl)-7H-pyrrolo [2,3-d] pyrimidin-2-amine 755.

White solid (1.1 mg, 0.003 mmol, 3.8% yield). ¹H NMR (499 MHz, DMSO-d₆) δppm 2.23 (3 H, s), 2.39-2.45 (4 H, m), 3.43-3.49 (4 H, m), 7.12 (1 H, dd, J=5.75, 1.64 Hz), 7.52 (1 H, d, J=1.10 Hz), 7.90 (1 H, s), 7.92 (1 H, d, J=4.11 Hz), 7.94 (1 H, s), 8.14 (1 H, dd, J=9.31, 1.64 Hz), 8.51 (1 H, s), 9.24 (1 H, s), 9.31 (1 H, s), 9.70 (1 H, s), 12.12 (1 H, s); ESIMS found for C22H₂₂Nio m/z 427.1 (M+1).

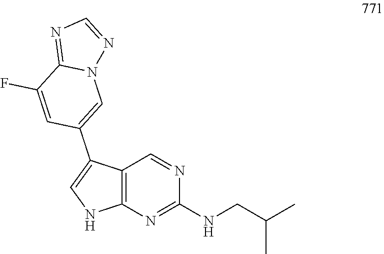 [SMILES: CC(C)CNc1ncc2c(-c3cc(F)c4ncnn4c3)c[nH]c2n1]

5-(8-Fluoro-[1,2,4]triazolo [1,5-a]pyridin-6-yl)-N-isobutyl-7H-pyrrolo [2,3-d] pyrimidin-2-amine 771.

White solid (3.9 mg, 0.012 mmol). ¹H NMR (400 MHz, DMSO-d₆) δppm 0.91 (6 H, d, J=6.60 Hz), 1.91 (1 H, dquin, J=13.43, 6.67, 6.67, 6.67, 6.67 Hz), 3.13 (2 H, br t, J=6.42 Hz), 6.94 (1 H, br s), 7.69 (1 H, d, J=1.59 Hz), 8.06 (1 H, d, J=11.74 Hz), 8.56 (1 H, s), 8.98 (1 H, s), 9.11 (1 H, s), 11.65 (1 H, br s); ESIMS found for C₁₆H₁₆FN₇ m/z 324.2 (M-1).

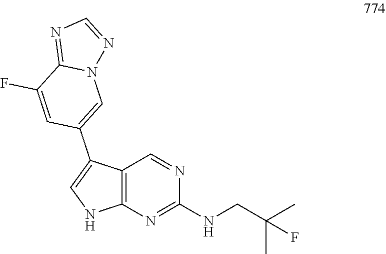 [SMILES: CC(C)(F)CNc1ncc2c(-c3cc(F)c4ncnn4c3)c[nH]c2n1]

N-(2-Fluoro-2-methylpropyl)-5-(8-fluoro-[1,2,4]triazolo [1,5-a] pyridin-6-yl)-7H-pyrrolo[2,3-d]pyrimidin-2-amine 774.

White solid (13.0 mg, 0.038 mmol). ¹H NMR (400 MHz, DMSO-d₆) δppm 1.35 (6 H, d, J=21.40 Hz), 3.59 (2 H, dd, J=19.01, 6.38 Hz), 6.96 (1 H, br t, J=5.82 Hz), 7.72 (1 H, d, J=2.38 Hz), 8.07 (1 H, d, J=11.76 Hz), 8.57 (1 H, s), 9.02 (1 H, s), 9.13 (1 H, s), 11.70 (1 H, br s); ESIMS found for C₁₆H₁₅F2N7 m/z 344.0 (M+1).

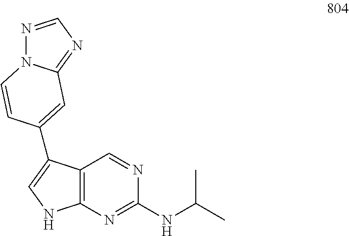 [SMILES: CC(C)Nc1ncc2c(-c3ccn4ncnc4c3)c[nH]c2n1]

5-([1,2,4] Triazolo [1,5-a] pyridin-7-yl)-N-i sopropyl-7H-pyrrolo [2,3-d] pyrimidin-2-amine 804.

Light brown solid (20 mg, 0.068 mmol, 34.3% yield). ¹1-1 NMR (499 MHz, DMSO-d₆) δppm 1.18 (6 H, d, J=6.30 Hz), 4.01-4.13 (1 H, m), 6.65 (1 H, br d, J=7.67 Hz), 7.64 (1 H, d, J=2.19 Hz), 7.87 (1 H, dd, J=9.31, 0.82 Hz), 8.06 (1 H, dd, J=9.17, 1.78 Hz), 8.48 (1 H, s), 8.95 (1 H, s), 9.19 (1 H, dd, J=1.64, 0.82 Hz), 11.62 (1 H, br s); ESIMS found for CisH₁₅N7 m/z 294.15 (M+1).

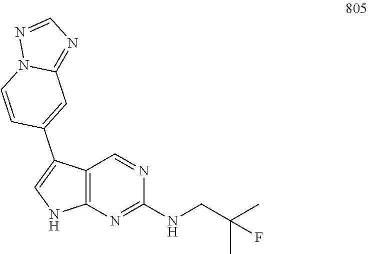 [SMILES: CC(C)(F)CNc1ncc2c(-c3ccn4ncnc4c3)c[nH]c2n1]

5-([1,2,4]Triazolo[1,5-a]pyridin-7-yl)-N-(2-fluoro-2-methylpropyl)-7H-pyrrolo [2,3-d]pyrimidin-2-amine 805.

White solid (46.6 mg, 0.143 mmol). ¹1-1 NMR (400 MHz, DMSO-d₆) δppm 1.35 (6 H, d, J=21.40 Hz), 3.59 (2 H, dd, J=19.01, 6.38 Hz), 6.99 (1 H, br t, J=5.38 Hz), 7.59 (1 H, dd, J=7.19, 1.81 Hz), 7.87 (1 H, d, J=2.38 Hz), 8.04 (1 H, d, J=1.00 Hz), 8.44 (1 H, s), 8.89 (1 H, d, J=7.25 Hz), 9.06 (1 H, s), 11.80 (1 H, br s); ESIMS found for C₁₆H₁₆FN₇ m/z 326.0 (M+1).

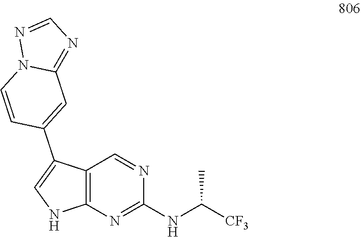 [SMILES: C[C@@H](Nc1ncc2c(-c3ccn4ncnc4c3)c[nH]c2n1)C(F)(F)F]

(R)-5-([1,2,4] Triazolo [1,5-a] pyridin-7-yl)-N-(1,1,1-trifluoropropan-2-yl)-7H-pyrrolo [2,3-d]pyrimidin-2-amine 806.

Beige solid (23 mg, 0.066 mmol, 22.6% yield). ¹H NMR (499 MHz, DMSO-d₆) δppm 1.36 (3 H, d, J=7.12 Hz), 4.89-5.01 (1 H, m), 7.49 (1 H, br d, J=8.76 Hz), 7.60 (1 H, dd, J=7.39, 1.92 Hz), 7.94 (1 H, s), 8.07 (1 H, d, J=1.10 Hz), 8.44 (1 H, s), 8.87-8.93 (1 H, m), 9.13 (1 H, s), 11.93 (1 H, br s); ESIMS found for C₁₅1-1₁₂F₃N₇ m/z 348.1 (M+1).

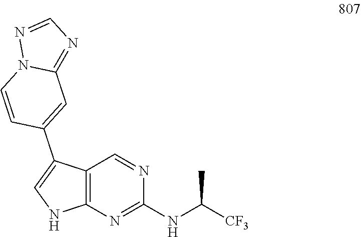 [SMILES: C[C@H](Nc1ncc2c(-c3ccn4ncnc4c3)c[nH]c2n1)C(F)(F)F]

(S)-5-([1,2,4] Triazolo [1,5-a] pyridin-7-yl)-N-(1,1,1-trifluoropropan-2-yl)-7H-pyrrolo [2,3-d]pyrimidin-2-amine 807.

White solid (12.3 mg, 0.035 mmol). ¹H NMR (400 MHz, DMSO-d₆) δppm 1.36 (3 H, d, J=7.00 Hz), 4.95 (1 H, br dd, J=14.63, 7.13 Hz), 7.49 (1 H, br d, J=8.76 Hz), 7.60 (1 H, dd, J=7.13, 1.13 Hz), 7.94 (1 H, s), 8.07 (1 H, s), 8.44 (1 H, s), 8.90 (1 H, d, J=7.13 Hz), 9.13 (1 H, s), 11.96 (1 H, br s); ESIMS found for C₁₅H₁₂F₃N₇ m/z 348.0 (M+1).

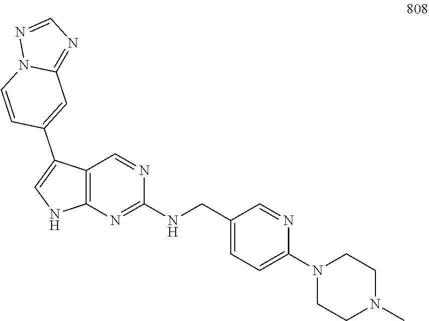 [SMILES: CN1CCN(c2ccc(CNc3ncc4c(-c5ccn6ncnc6c5)c[nH]c4n3)cn2)CC1]

5-([1,2,4]Triazolo[1,5-a]pyridin-7-yl)-N-46-(4-methylpiperazin-1-yl)pyridin-3-yl)methyl)-7H-pyrrolo [2,3-d] pyrimidin-2-amine 808.

Off-white solid (5.8 mg, 0.013 mmol, 14.8% yield). ¹H NMR (499 MHz, DMSO-d₆) δppm 2.19 (3 H, s), 2.33-2.39 (4 H, m), 3.38-3.45 (4 H, m), 4.40 (2 H, d, J=6.30 Hz), 6.77 (1 H, d, J=8.76 Hz), 7.35 (1 H, br d, J=4.11 Hz), 7.55 (1 H, dd, J=8.76, 2.46 Hz), 7.58 (1 H, dd, J=7.39, 1.92 Hz), 7.86 (1 H, s), 8.03 (1 H, d, J=1.10 Hz), 8.13 (1 H, d, J=2.19 Hz), 8.43 (1 H, s), 8.88 (1 H, d, J=7.12 Hz), 9.06 (1 H, s), 11.80 (1 H, br s); ESIMS found for C₂₃H₂₄Nio m/z 441.1 (M+1).

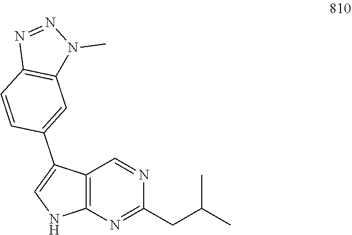 [SMILES: CC(C)Cc1ncc2c(-c3ccc4nnn(C)c4c3)c[nH]c2n1]

6-(2-Isobutyl-7H-pyrrolo[2,3-d]pyrimidin-5-yl)-1-methyl-1H-benzo[d] [1,2,3]triazole 810.

Off-white solid (20 mg, 0.065 mmol, 16.8% yield). ¹H NMR (499 MHz, DMSO-d₆) δppm 0.94 (6 H, d, J=6.57 Hz), 2.30 (1 H, dquin, J=13.69, 6.84, 6.84, 6.84, 6.84 Hz), 2.81 (2 H, d, J=7.12 Hz), 4.38 (3 H, s), 7.84 (1 H, dd, J=8.62, 1.51 Hz), 8.06 (1 H, d, J=11.50 Hz), 8.06 (1 H, s), 8.17 (1 H, s), 9.50 (1 H, s), 12.28 (1 H, br s); ESIMS found for C₁₇H₁₈N₆ m/z 307.2 (M+1).

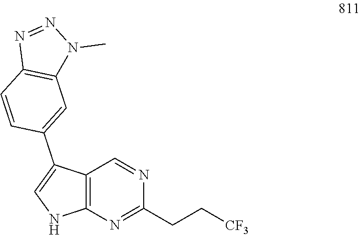 [SMILES: Cn1nnc2ccc(-c3c[nH]c4nc(CCC(F)(F)F)ncc34)cc21]

1-Methyl-6-(2-(3,3,3-trifluoropropyl)-7H-pyrrolo [2,3-d]pyrimidin-5-yl)-1H-benzo[d][1,2,3]triazole 811.

Off-white solid (8 mg, 0.023 mmol, 19.3% yield). ¹H NMR (500 MHz, DMSO-d₆) δppm 2.81-2.95 (2 H, m), 3.16-3.25 (2 H, m), 4.38 (3 H, s), 7.85 (1 H, dd, J=8.78, 1.37 Hz), 8.06 (1 H, d, J=8.78 Hz), 8.12 (1 H, d, J=1.37 Hz), 8.17 (1 H, s), 9.54 (1 H, s), 12.40 (1 H, br s); ESIMS found for C₁₆K₃F₃N₆ m/z 347.1 (M+1).

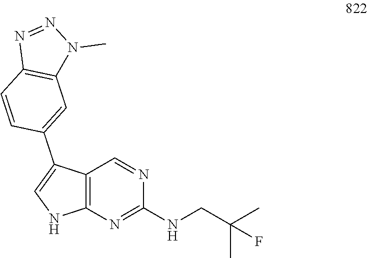 [SMILES: Cn1nnc2ccc(-c3c[nH]c4nc(NCC(C)(C)F)ncc34)cc21]

N-(2-Fluoro-2-methylpropyl)-5-(1-methyl-1H-benzo[d][1,2,3]triazol-6-yl)-7H-pyrrolo[2,3-d]pyrimidin-2-amine 822.

White solid (95.0 mg, 0.280 mmol). ¹H NMR (400 MHz, DMSO-d₆) δppm 1.35 (6 H, d, J=21.40 Hz), 3.61 (2 H, br dd, J=19.01, 6.38 Hz), 4.36 (3 H, s), 6.90 (1 H, brt, J=6.19 Hz), 7.64 (1 H, d, J=1.75 Hz), 7.77 (1 H, dd, J=8.82, 0.94 Hz), 8.00 (1 H, d, J=8.63 Hz), 8.04 (1 H, s), 9.12 (1 H, s), 11.62 (1 H, br s); ESIMS found for C₁₇HisFN₇ m/z 340.2 (M+1).

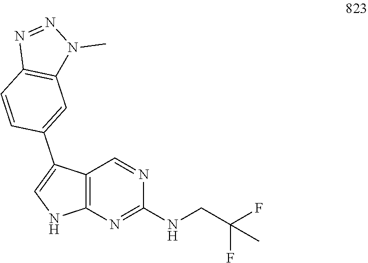 [SMILES: Cn1nnc2ccc(-c3c[nH]c4nc(NCC(C)(F)F)ncc34)cc21]

N-(2,2-Difluoropropyl)-5-(1-methyl-1H-benzo[d][1,2,3]triazol-6-yl)-7H-pyrrolo [2,3-d]pyrimidin-2-amine 823.

White solid (15.94 mg, 0.047 mmol). ¹H NMR (400 MHz, DMSO-d₆) δppm 1.63 (3 H, t, J=19.01 Hz), 3.85 (2 H, td, J=13.66, 6.66 Hz), 7.20 (1 H, br t, J=6.42 Hz), 7.68 (1 H, d, J=2.20 Hz), 7.77 (1 H, dd, J=8.74, 1.41 Hz), 8.00 (1 H, d, J=8.68 Hz), 8.05 (1 H, s), 9.15 (1 H, s), 11.69 (1 H, br s); ESIMS found for C₁₆H₁₅F₂N₇ m/z 344.3 (M+1).

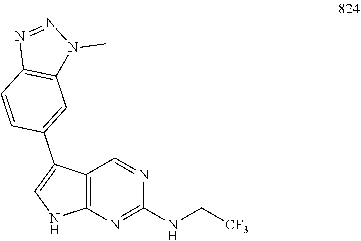 [SMILES: Cn1nnc2ccc(-c3c[nH]c4nc(NCC(F)(F)F)ncc34)cc21]

5-(1-Methyl-1H-benzo[d][1,2,3]triazol-6-yl)-N-(2,2,2-trifluoroethyl)-7H-pyrrolo [2,3-d]pyrimidin-2-amine 824.

White solid (11.21 mg, 0.032 mmol). ¹H NMR (400 MHz, DMSO-d₆) δppm 4.12-4.27 (2 H, m), 4.36 (3 H, s), 7.45 (1 H, br t, J=6.44 Hz), 7.72 (1 H, s), 7.78 (1 H, d, J=8.63 Hz), 8.01 (1 H, d, J=8.63 Hz), 8.06 (1 H, s), 9.19 (1 H, s), 11.77 (1 H, br s); ESIMS found for CisH₁₂F3N7 m/z 348.1 (M+1).

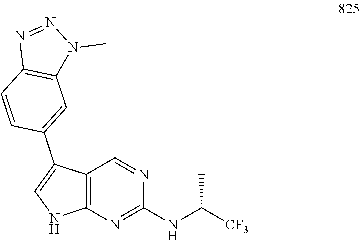 [SMILES: C[C@@H](Nc1ncc2c(-c3ccc4nnn(C)c4c3)c[nH]c2n1)C(F)(F)F]

(R)-5-(1-Methyl-1H-benzo[d] [1,2,3]triazol-6-yl)-N-(1,1,1-trifluoropropan-2-yl)-7H-pyrrolo [2,3-d] pyrimidin-2-amine 825.

White solid (35.03 mg, 0.097 mmol). ¹H NMR (400 MHz, DMSO-d₆) δppm 1.37 (3 H, d, J=6.97 Hz), 4.36 (3 H, s), 4.89-5.05 (1 H, m), 7.40 (1 H, br d, J=9.17 Hz), 7.71 (1 H, d, J=2.20 Hz), 7.78 (1 H, dd, J=8.74, 1.53 Hz), 8.01 (1 H, d, J=8.80 Hz), 8.06 (1 H, s), 9.17 (1 H, s), 11.75 (1 H, br s); ESIMS found for C₁₆H₁₄F₃N₇ m/z 362.1 (M+1).

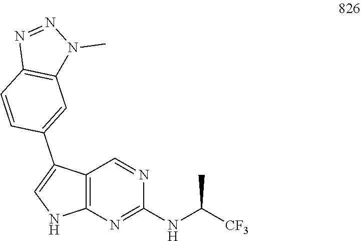 [SMILES: C[C@H](Nc1ncc2c(-c3ccc4nnn(C)c4c3)c[nH]c2n1)C(F)(F)F]

(S)-5-(1-Methyl-1H-benzo[d] [1,2,3]triazol-6-yl)-N-(1,1,1-trifluoropropan-2-yl)-7H-pyrrolo [2,3-d] pyrimidin-2-amine 826.

White solid (64.46 mg, 0.179 mmol). ¹H NMR (400 MHz, DMSO-d₆) δppm 1.37 (3 H, d, J=6.97 Hz), 4.36 (3 H, s), 4.90-5.05 (1 H, m), 7.40 (1 H, br d, J=9.05 Hz), 7.71 (1 H, s), 7.78(1 H, dd, J=8.74, 1.41 Hz), 8.01 (1 H, d, J=8.68 Hz), 8.06 (1 H, s), 9.17 (1 H, s), 11.75 (1 H, br s); ESIMS found for C₁₆H₁₄F₃N₇ m/z 362.2 (M+1).

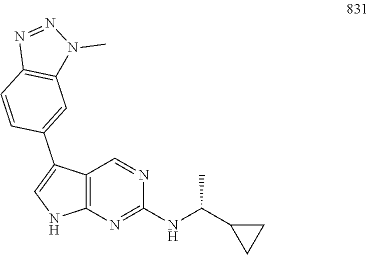 [SMILES: C[C@@H](Nc1ncc2c(-c3ccc4nnn(C)c4c3)c[nH]c2n1)C1CC1]

(R)-N-(1—Cyclopropylethyl)-5-(1-methyl-1H-benzo[d][1,2,3]triazol-6-yl)-7H-pyrrolo [2,3-d]pyrimidin-2-amine 831.

White solid (24.58 mg, 0.074 mmol). ¹H NMR (400 MHz, DMSO-d₆) δppm 0.12-0.22 (1 H, m), 0.30-0.48 (3 H, m), 0.94-1.08 (1 H, m), 1.22 (3 H, d, J=6.60 Hz), 3.52-3.67 (1 H, m), 4.35 (3 H, s), 6.67 (1 H, br d, J=8.44 Hz), 7.60 (1 H, d, J=2.20 Hz), 7.75 (1 H, dd, J=8.68, 1.47 Hz), 7.99 (1 H, d, J=8.93 Hz), 8.01 (1 H, s), 9.07 (1 H, s), 11.52 (1 H, br s); ESIMS found for C₁₅H₁₉N₇ m/z 334.2 (M+1).

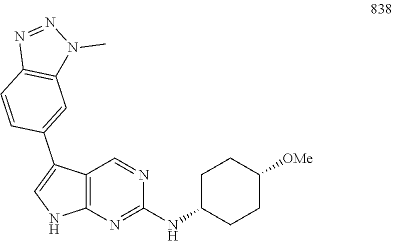 [SMILES: CO[C@H]1CC[C@@H](Nc2ncc3c(-c4ccc5nnn(C)c5c4)c[nH]c3n2)CC1]

N-(cis-4-Methoxycyclohexyl)-5-(1-methyl-1H-benzo[d] [1,2,3]triazol-6-yl)-7H-pyrrolo [2,3-d]pyrimidin-2-amine 838.

White solid (19.20 mg, 0.051 mmol). ¹H NMR (400 MHz, DMSO-d₆) δppm 1.41-1.53 (2 H, m), 1.54-1.64 (2 H, m), 1.69 (2 H, br dd, J=11.51, 1.38 Hz), 1.86 (2 H, br dd, J=9.44, 3.94 Hz), 3.33-3.40 (2 H, m), 3.74-3.87 (1 H, m), 4.35 (3 H, s), 6.62-6.71 (1 H, m), 7.60 (1 H, s), 7.76 (1 H, br d, J=7.63 Hz), 7.99 (1 H, br d, J=9.01 Hz), 8.02 (1 H, br s), 9.09 (1 H, s), 11.56 (1 H, br s); ESIMS found for C₂₀1⁻1₂₃N₇O m/z 378.2 (M+1).

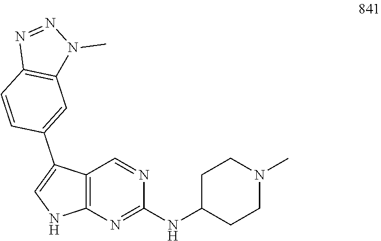 [SMILES: CN1CCC(Nc2ncc3c(-c4ccc5nnn(C)c5c4)c[nH]c3n2)CC1]

5-(1-Methyl-1H-benzo[d][1,2,3]triazol-6-yl)-N-(1-methylpiperidin-4-yl)-7H-pyrrolo [2,3-d]pyrimidin-2-amine 841.

White solid (1.59 mg, 0.004 mmol). ¹1-1 NMR (400 MHz, DMSO-d₆) δppm 1.46-1.62 (2 H, m), 1.89 (2 H, br d, J=11.76 Hz), 1.95 (2 H, br t, J=11.76 Hz), 2.17 (3 H, s), 2.76 (2 H, br d, J=11.26 Hz), 3.66-3.80 (1 H, m), 4.35 (3 H, s), 6.65 (1 H, br d, J=7.63 Hz), 7.61 (1 H, s), 7.76 (1 H, d, J=8.76 Hz), 7.99 (1 H, d, J=8.76 Hz), 8.02 (1 H, s), 9.10 (1 H, s), 11.58 (1 H, br s); ESIMS found for C₁₉H₂₂N8 m/z 363.2 (M+1).

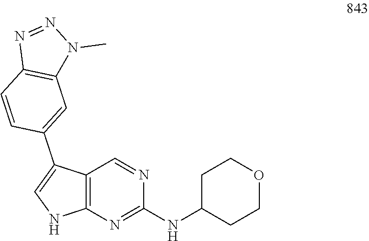 [SMILES: Cn1nnc2ccc(-c3c[nH]c4nc(NC5CCOCC5)ncc34)cc21]

5-(1-Methyl-1H-benzo[d][1,2,3]triazol-6-yl)-N-(tetrahydro-2H-pyran-4-yl)-7H-pyrrolo [2,3-d]pyrimidin-2-amine 843.

White solid (10.6 mg, 0.030 mmol). ¹1-1 NMR (400 MHz, DMSO-d₆) δppm 1.47-1.63 (2 H, m), 1.89 (2 H, br dd, J=12.51, 1.88 Hz), 3.38-3.45 (2 H, m), 3.90 (2 H, br d, J=11.26 Hz), 3.93-4.04 (1 H, m), 4.35 (3 H, s), 6.78 (1 H, br d, J=7 .7 5 Hz), 7.62 (1 H, d, J=1.38 Hz), 7.76 (1 H, dd, J=8.76, 0.88 Hz), 7.99 (1 H, d, J=8.76 Hz), 8.02 (1 H, s), 9.11 (1 H, s), 11.60 (1 H, br s); ESIMS found for C₁₈H₁₉N₇O m/z 350.3 (M+1).

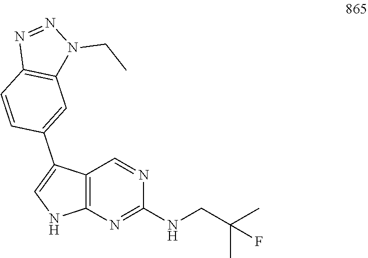 [SMILES: CCn1nnc2ccc(-c3c[nH]c4nc(NCC(C)(C)F)ncc34)cc21]

5-(1-Ethyl-1H-benzo[d][1,2,3]triazol-6-yl)-N-(2-fluoro-2-methylpropyl)-7H-pyrrolo[2,3-d]pyrimidin-2-amine 865.

White solid (26.66 mg, 0.075 mmol). ¹H NMR (400 MHz, DMSO-d₆) δppm 1.35 (6 H, d, J=21.40 Hz), 1.54 (3 H, t, J=7.27 Hz), 3.61 (2 H, dd, J=19.07, 6.36 Hz), 4.81 (2 H, q, J=7.21 Hz), 6.88 (1 H, br t, J=6.36 Hz), 7.64 (1 H, d, J=2.32 Hz), 7.76 (1 H, dd, J=8.80, 1.47 Hz), 8.01 (1 H, d, J=8.68 Hz), 8.06 (1 H, s), 9.09 (1 H, s), 11.60 (1 H, br s); ESIMS found for C₁₈H₂₀FN₇ m/z 354.2 (M+1).

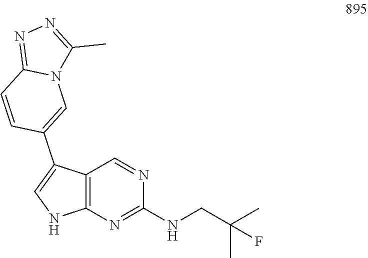 [SMILES: Cc1nnc2ccc(-c3c[nH]c4nc(NCC(C)(C)F)ncc34)cn12]

N-(2-Fluoro-2-methylpropyl)-5-(3-methyl41,2,41triazolo [4,3-a]pyridin-6-yl)-7H-pyrrolo [2,3-d] pyrimidin-2-amine 895.

White solid (3,2 mg, 0.009 mmol). ¹H NMR (400 MHz, DMSO-d₆) δppm 1.35 (6 H, d, J=21.40 Hz), 2.77 (3 H, s), 3.60 (2 H, br dd, J=19.32, 6.36 Hz), 6.92 (1 H, br t, J=6.05 Hz), 7.63 (1 H, d, J=2.20 Hz), 7.69-7.79 (2 H, m), 8.42 (1 H, s), 9.02 (1 H, s), 11.61 (1 H, br s); ESIMS found for C₁₇HisFN7 m/z 340.2 (M+1).

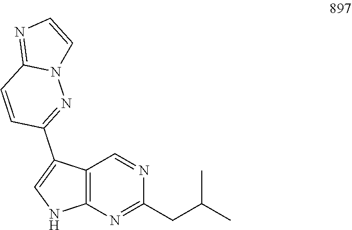 [SMILES: CC(C)Cc1ncc2c(-c3ccc4nccn4n3)c[nH]c2n1]

5-(Imidazo[1,2-b]pyridazin-6-yl)-2-isobutyl-7H-pyrrolo[2,3-d]pyrimidine 897.

Off-white solid (70 mg, 0.239 mmol, 48.2% yield). ¹H NMR (499 MHz, DMSO-d₆) δppm 0.93 (7 H, d, J=6.57 Hz), 2.28 (1 H, dquin, J=13.67, 6.78, 6.78, 6.78, 6.78 Hz), 2.82 (2 H, d, J=7.12 Hz), 7.72 (1 H, d, J=1.09 Hz), 7.83 (1 H, d, J=9.58 Hz), 8.13 (1 H, d, J=9.58 Hz), 8.35 (1 H, s), 8.47 (1 H, s), 9.67 (1 H, s), 12.48 (1 H, s); ESIMS found for C₁₆H₁₆N₆ m/z 293.1 (M+1).

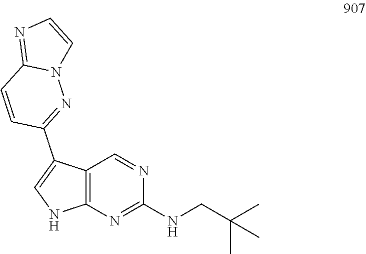 [SMILES: CC(C)(C)CNc1ncc2c(-c3ccc4nccn4n3)c[nH]c2n1]

5-(Imidazo [1,2-13,] pyridazin-6-yl)-N-neopentyl-7H-pyrrolo [2,3-d]pyrimidin-2-amine 907.

White solid (33.77 mg, 0.105 mmol). ¹H NMR (400 MHz, DMSO-d₆) δppm 0.93 (9 H, s), 3.23 (2 H, br d, J=6.11 Hz), 6.97 (1 H, br s), 7.28-7.35 (1 H, m), 7.56-7.64 (1 H, m), 7.78 (1 H, d, J=9.54 Hz), 8.04-8.11 (2 H, m), 9.25 (1 H, s), 11.89 (1 H, br s); ESIMS found for C₁₇H₁₉N7 m/z 322.3 (M+1).

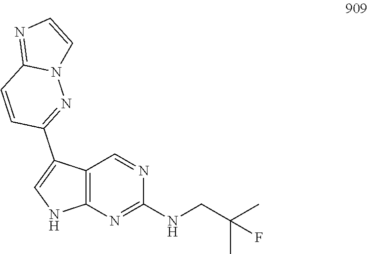 [SMILES: CC(C)(F)CNc1ncc2c(-c3ccc4nccn4n3)c[nH]c2n1]

N-(2-Fluoro-2-methylpropyl)-5-(imidazo[1,2-b]pyridazin-6-yl)-7H-pyrrolo [2,3-d]pyrimidin-2-amine 909.

White solid (35.12 mg, 0.108 mmol). ¹H NMR (400 MHz, DMSO-d₆) δppm 1.35 (6 H, d, J=21.40 Hz), 3.60 (2 H, br dd, J=18.83, 6.60 Hz), 7.00 (1 H, br t, J=6.05 Hz), 7.68 (1 H, d, J=1.10 Hz), 7.74 (1 H, d, J=9.66 Hz), 8.03-8.08 (2 H, m), 8.30 (1 H, s), 9.28 (1 H, s), 11.83 (1 H, br s); ESIMS found for C₁₆H₁₆FN₇ m/z 326.2 (M+1).

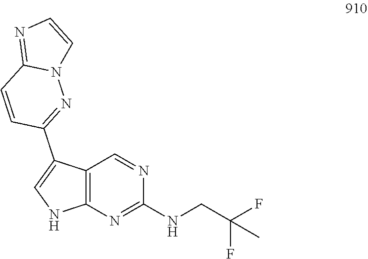 [SMILES: CC(F)(F)CNc1ncc2c(-c3ccc4nccn4n3)c[nH]c2n1]

N-(2,2-Difluoropropyl)-5-(imidazo [1,2-13,] pyridazin-6-yl)-7H-pyrrolo [2,3-d] pyrimidin-2-amine 910.

White solid (0.37 mg, 0.0011 mmol). ¹H NMR (400 MHz, DMSO-d₆) δppm 1.63 (3 H, t, J=18.95 Hz), 3.83 (2 H, td, J=13.85, 6.54 Hz), 7.29 (1 H, br t, J=5.99 Hz), 7.69 (1 H, d, J=0.73 Hz), 7.75 (1 H, d, J=9.66 Hz), 8.06 (1 H, d, J=9.54 Hz), 8.10 (1 H, s), 8.31 (1 H, s), 9.32 (1 H, s), 11.90 (1 H, br s); ESIMS found for C₁₅1-1₁₃F₂N₇ m/z 330.0 (M+1).

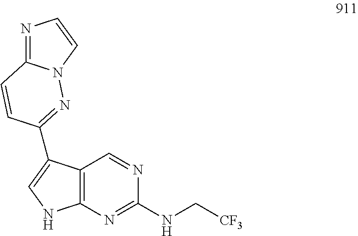 [SMILES: FC(F)(F)CNc1ncc2c(-c3ccc4nccn4n3)c[nH]c2n1]

5-(Imidazo [1,2-b] pyridazin-6-yl)-N-(2,2,2-trifluoroethyl)-7H-pyrrolo [2,3-d] pyrimidin-2-amine 911.

White solid (4.38 mg, 0.013 mmol'H NMR (400 MHz, DMSO-d₆) δppm 4.11-4.26 (2 H, m), 7.59 (1 H, br t, J=6.42 Hz), 7.83 (1 H, s), 7.89 (1 H, d, J=9.54 Hz), 8.11-8.17 (1 H, m), 8.20 (1 H, d, J=2.45 Hz), 8.41 (1 H, s), 9.37 (1 H, s), 12.07 (1 H, br s); ESIMS found for C₁₄H₁₀F3N7 m/z 334.1 (M+1).

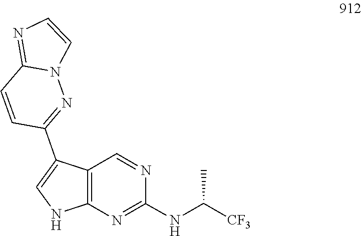 [SMILES: C[C@@H](Nc1ncc2c(-c3ccc4nccn4n3)c[nH]c2n1)C(F)(F)F]

(R)-5-(Imidazo [1,2-b]pyridazin-6-yl)-N-(1,1,1-trifluoropropan-2-yl)-7H-pyrrolo [2,3-d] pyrimidin-2-amine 912.

White solid (5.5 mg, 0.016 mmol). ¹H NMR (400 MHz, DMSO-d₆) δppm 1.37 (3 H, d, J=7.09 Hz), 4.95 (1 H, br dd, J=15.04, 6.97 Hz), 7.50 (1 H, br d, J=8.31 Hz), 7.69 (1 H, d, J=0.98 Hz), 7.75 (1 H, d, J=9.54 Hz), 8.07 (1 H, d, J=9.54 Hz), 8.13 (1 H, s), 8.31 (1 H, s), 9.34 (1 H, s), 11.95 (1 H, br s); ESIMS found for CisH₁₂F₃N₇ m/z 348.1 (M+1).

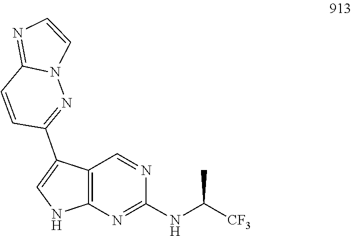 [SMILES: C[C@H](Nc1ncc2c(-c3ccc4nccn4n3)c[nH]c2n1)C(F)(F)F]

(S)-5-(Imidazo [1,2-b]pyridazin-6-yl)-N-(1,1,1-trifluoropropan-2-yl)-7H-pyrrolo [2,3-d] pyrimidin-2-amine 913.

White solid (13.66 mg, 0.039 mmol). ¹H NMR (400 MHz, DMSO-d₆) δppm 1.37 (3 H, d, J=7.00 Hz), 4.87-5.05 (1 H, m), 7.50 (1 H, br d, J=9.76 Hz), 7.69 (1 H, s), 7.75 (1 H, d, J=9.63 Hz), 8.07 (1 H, d, J=9.63 Hz), 8.13 (1 H, s), 8.31 (1 H, s), 9.34 (1 H, s), 11.96 (1 H, br s); ESIMS found for CisH₁₂F3N7 m/z 347.9 (M+1).

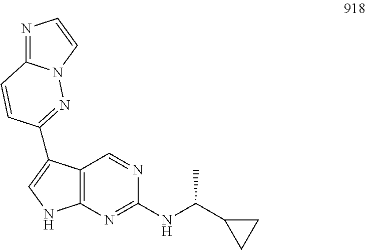 [SMILES: C[C@@H](Nc1ncc2c(-c3ccc4nccn4n3)c[nH]c2n1)C1CC1]

(R)-N-(1—Cyclopropylethyl)-5-(imidazo[1,2-b]pyridazin-6-yl)-7H-pyrrolo [2,3-d]pyrimidin-2-amine 918.

White solid (2.91 mg, 0.009 mmol). ¹1-1 NMR (400 MHz, DMSO-d₆) δppm 0.09-0.23 (1 H, m), 0.30-0.48 (3 H, m), 0.95-1.09 (1 H, m), 1.22 (3 H, d, J=6.60 Hz), 3.51-3.64 (1 H, m), 6.79 (1 H, br d, J=8.31 Hz), 7.68 (1 H, d, J=0.98 Hz), 7.73 (1 H, d, J=9.66 Hz), 8.00-8.06 (2 H, m), 8.29 (1 H, s), 9.24 (1 H, s), 11.74 (1 H, br s); ESIMS found for C₁₇H₁₇N7 m/z 320.2 (M+1).

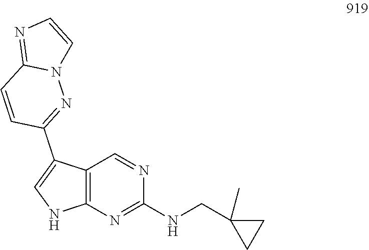 [SMILES: CC1(CNc2ncc3c(-c4ccc5nccn5n4)c[nH]c3n2)CC1]

5-(Imidazo [1,2-b] pyridazin-6-yl)-N-((1-methylcyclopropyl)methyl)-7H-pyrrolo [2,3-d] pyrimidin-2-amine 919.

White solid (7.53 mg, 0.024 mmol). ¹1-1 NMR (400 MHz, DMSO-d₆) δppm 0.18-0.26 (2 H, m), 0.46-0.53 (2 H, m), 1.09 (3 H, s), 3.30 (2 H, d, J=4.88 Hz), 6.85-6.95 (1 H, m), 7.68 (1 H, d, J=0.75 Hz), 7.73 (1 H, d, J=9.51 Hz), 8.03 (1 H, s), 8.05 (1 H, d, J=8.08 Hz), 8.30 (1 H, s), 9.25 (1 H, s), 11.76 (1 H, br s); ESIMS found for C₁₇H₁₇N₇ m/z 320.2 (M+1).

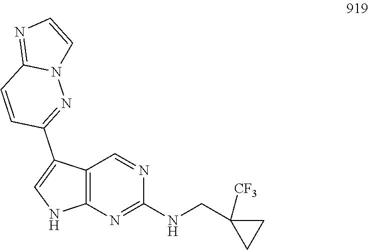 [SMILES: FC(F)(F)C1(CNc2ncc3c(-c4ccc5nccn5n4)c[nH]c3n2)CC1]

5-(Imidazo [1,2-b] pyridazin-6-yl)-N-41-(trifluo romethyl)cyclopropyl) methyl)-7H-pyrrolo [2,3-d]pyrimidin-2-amine 920.

White solid (17.5 mg, 0.047 mmol). ¹1-1 NMR (400 MHz, DMSO-d₆) δppm 0.80-0.89 (2 H, m), 0.94 (2 H, br s), 3.76 (2 H, d, J=6.50 Hz), 6.85-6.96 (1 H, m), 7.66 (1 H, d, J=0.75 Hz), 7.72 (1 H, d, J=9.51 Hz), 8.01 (1 H, d, J=9.51 Hz), 8.05 (1 H, s), 8.28 (1 H, s), 9.25 (1 H, s); ESIMS found for C₁₇H₁₄F₃N₇ m/z 374.1 (M+1).

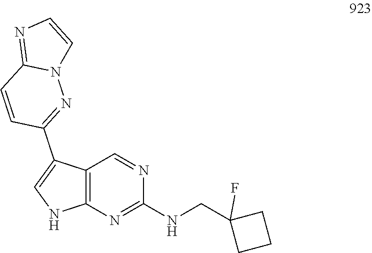 [SMILES: FC1(CNc2ncc3c(-c4ccc5nccn5n4)c[nH]c3n2)CCC1]

N-((l-Fluorocyclobutyl)methyl)-5-(imidazo[1,2-b]pyridazin-6-yl)-7H-pyrrolo [2,3-d]pyrimidin-2-amine 923.

White solid (10.17 mg, 0.030 mmol). ¹H NMR (400 MHz, DMSO-d₆) δppm 1.49-1.63 (1 H, m), 1.71-1.84 (1 H, m), 2.10-2.29 (4 H, m), 3.74 (2 H, dd, J=22.60, 6.08 Hz), 6.99-7.10 (1 H, m), 7.68 (1 H, d, J=0.98 Hz), 7.74 (1 H, d, J=9.54 Hz), 8.06 (1 H, d, J=12.35 Hz), 8.07 (1 H, s), 8.31 (1 H, s), 9.28 (1 H, s), 11.82 (1 H, br s); ESIMS found for C₁₇H₁₆FN₇ m/z 338.2 (M+1).

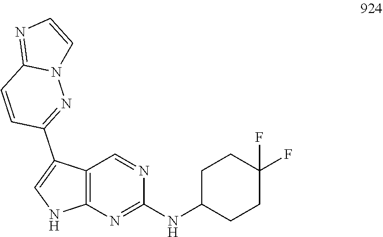 [SMILES: FC1(F)CCC(Nc2ncc3c(-c4ccc5nccn5n4)c[nH]c3n2)CC1]

N-(4,4-Difluorocyclohexyl)-5-(imidazo [1,2-b] pyridazin-6-yl)-7H-pyrrolo [2,3-d] pyrimidin-2-amine 924.

White solid (10.39 mg, 0.028 mmol). ¹H NMR (400 MHz, DMSO-d₆) δppm 1.57-1.75 (2 H, m), 1.81-2.03 (4 H, m), 2.04-2.19 (2 H, m), 3.87-4.03 (1 H, m), 6.97 (1 H, br d, J=7.63 Hz), 7.68 (1 H, d, J=0.88 Hz), 7.74 (1 H, d, J=9.63 Hz), 8.04 (1 H, br d, J=9.51 Hz), 8.05 (1 H, s), 8.30 (1 H, s), 9.29 (1 H, s), 11.82 (1 H, br s); ESIMS found for CisH₁₇F₂N₇ m/z 370.2 (M+1).

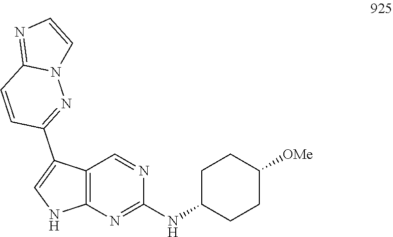 [SMILES: CO[C@H]1CC[C@@H](Nc2ncc3c(-c4ccc5nccn5n4)c[nH]c3n2)CC1]

5-(Imidazo [1,2-b] pyridazin-6-yl)-N-(cis-4-methoxycyclohexyl)-7H-pyrrolo [2,3-d]pyrimidin-2-amine 925.

White solid (10.7 mg, 0.029 mmol). ¹H NMR (400 MHz, DMSO-d₆) δppm 1.42-1.53 (2 H, m), 1.55-1.65 (2 H, m), 1.65-1.73 (2 H, m), 1.80-1.92 (2 H, m), 3.23 (3 H, s),

3.34-3.38 (2 H, m), 3.73-3.87 (1 H, m), 6.71-6.81 (1 H, m), 7.67 (1 H, d, J=1.00 Hz), 7.72 (1 H, d, J=9.51 Hz), 8.01-8.05 (2 H, m), 8.28 (1 H, s), 9.25 (1 H, s), 11.79 (1 H, br s); ESIMS found for C₁₉H₂iN70 m/z 364.2 (M+1)

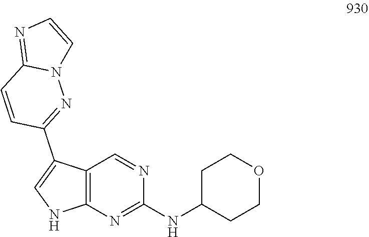 [SMILES: c1cn2nc(-c3c[nH]c4nc(NC5CCOCC5)ncc34)ccc2n1]

5-(Imidazo[1,2-b]pyridazin-6-yl)-N-(tetrahydro-2H-pyran-4-yl)-7H-pyrrolo [2,3-d] pyrimidin-2-amine 930.

White solid (46.01 mg, 0.137 mmol). ¹H NMR (400 MHz, DMSO-d₆) δppm 1.45-1.60 (2 H, m), 1.89 (2 H, br dd, J=12.41, 2.26 Hz), 3.36-3.46 (2 H, m), 3.84-3.93 (2 H, m), 3.94-4.03 (1 H, m), 6.78 (1 H, br d, J=6.72 Hz), 7.66 (1 H, d, J=1.10 Hz), 7.71 (1 H, d, J=9.66 Hz), 8.01 (1 H, d, J=9.54 Hz), 8.03 (1 H, s), 8.27 (1 H, s), 9.25 (1 H, s), 11.76 (1 H, br s); ESIMS found for C₁₇H₁₇N70 m/z 336.1 (M+1).

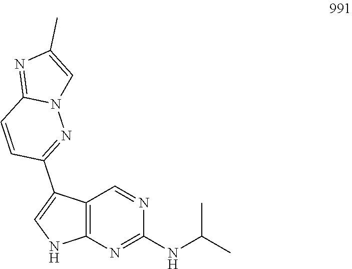 [SMILES: Cc1cn2nc(-c3c[nH]c4nc(NC(C)C)ncc34)ccc2n1]

N-Isopropyl-5-(2-methylimidazo[1,2-b]pyridazin-6-yl)-7H-pyrrolo[2,3-d]pyrimidin-2-amine 991.

Yellow solid (13 mg, 0.042 mmol, 48.5% yield). ¹H NMR (499 MHz, DMSO-d₆) δppm 1.18 (6 H, d, J=6.30 Hz), 2.38 (3 H, s), 4.07 (1 H, dq, J=13.48, 6.64 Hz), 6.72 (1 H, br d, J=6.84 Hz), 7.67 (1 H, d, J=9.58 Hz), 7.91 (1 H, d, J=9.31 Hz), 7.99 (1 H, d, J=2.46 Hz), 8.04 (1 H, s), 9.22 (1 H, s), 11.76 (1 H, br s); ESIMS found for C₁₆H₁₇N₇ m/z 308.2 (M+1).

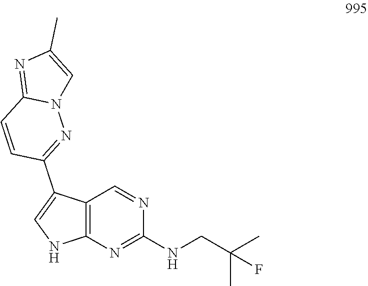 [SMILES: Cc1cn2nc(-c3c[nH]c4nc(NCC(C)(C)F)ncc34)ccc2n1]

N-(2-Fluoro-2-methylpropyl)-5-(2-methylimidazo [1,2-b] pyridazin-6-yl)-7H-pyrrolo [2,3-d] pyrimidin-2-amine 995.

White solid (25.1 mg, 0.074 mmol). ¹H NMR (400 MHz, DMSO-d₆) δppm 1.35 (6 H, d, J=21.40 Hz), 2.38 (3 H, s), 3.60 (2 H, br dd, J=19.07, 6.36 Hz), 6.98 (1 H, brt, J=5.69 Hz), 7.67 (1 H, d, J=9.54 Hz), 7.91 (1 H, d, J=9.54 Hz), 8.01 (1 H, s), 8.04 (1 H, s), 9.24 (1 H, s), 11.79 (1 H, br s); ESIMS found for C₁₇HisFN₇ m/z 340.1 (M+1).

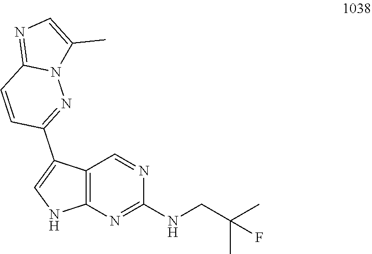 [SMILES: Cc1cnc2ccc(-c3c[nH]c4nc(NCC(C)(C)F)ncc34)nn12]

N-(2-Fluoro-2-methylpropyl)-5-(3-methylimidazo [1,2-b] pyridazin-6-yl)-7H-pyrrolo [2,3-d] pyrimidin-2-amine 1038.

White solid (3.02 mg, 0.009 mmol). ¹H NMR (400 MHz, DMSO-d₆) δppm 1.35 (6 H, d, J=21.40 Hz), 2.59 (3 H, s), 3.61 (2 H, br dd, J=19.07, 6.24 Hz), 6.96 (1 H, brt, J=6.11 Hz), 7.52 (1 H, s), 7.70 (1 H, d, J=9.29 Hz), 8.01 (1 H, d, J=9.78 Hz), 8.06 (1 H, s), 9.28 (1 H, s), 11.81 (1 H, br s); ESIMS found for C₁₇HisFN₇ m/z 340.1 (M+1).

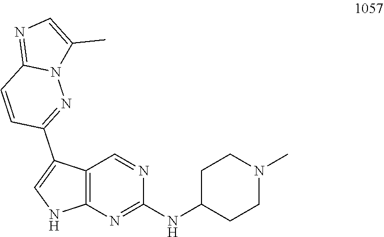 [SMILES: Cc1cnc2ccc(-c3c[nH]c4nc(NC5CCN(C)CC5)ncc34)nn12]

5-(3-Methylimidazo [1,2-b] pyridazin-6-yl)-N-(1-methylpipe ridin-4-yl)-7H-pyrrolo [2,3-d] pyrimidin-2-amine 1057.

White solid (4.05 mg, 0.011 mmol). ¹H NMR (400 MHz, DMSO-d₆) δppm 1.53 (2 H, qd, J=11.67, 3.38 Hz), 1.88 (2 H, br d, J=10.51 Hz), 1.95 (2 H, br t, J=11.51 Hz), 2.16 (3 H, s), 2.76(2 H, br d, J=11.51 Hz), 3.64-3.77 (1 H, m), 6.69 (1 H, br d, J=7 .7 5 Hz), 7.66 (1 H, s), 7.71 (1 H, d, J=9.63 Hz), 8.01 (1 H, br d, J=9.51 Hz), 8.01 (1 H, s), 8.27(1 H, s), 9.24(1 H, s); ESIMS found for C₁₈H₂₀N8 m/z 349.1 (M+1).

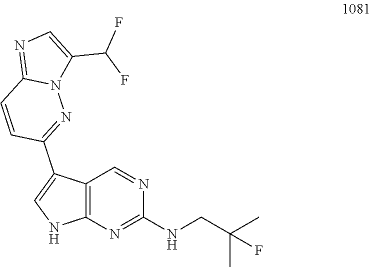 [SMILES: CC(C)(F)CNc1ncc2c(-c3ccc4ncc(C(F)F)n4n3)c[nH]c2n1]

5-(3-(Difluoromethyl)imidazo [1,2-b]pyridazin-6-yl)-N-(2-fluoro-2-methylpropyl)-7H-pyrrolo [2,3-d]pyrimidin-2-amine 1081.

White solid (5.2 mg, 0.014 mmol). ¹1-1 NMR (400 MHz, MHz, DMSO-d₆) 6 ppm 1.35 (6 H, d, J=21.40 Hz), 3.60 (8 H, br dd, J=19.32, 6.44 Hz), 7.00 (1 H, br t, J=5.94 Hz), 7.65 (1 H, t, J=53.36 Hz), 7.93 (1 H, d, J=9.63 Hz), 7.99 (1 H, s), 8.15 (1 H, s), 8.18 (1 H, d, J=9.76 Hz), 9.24 (1 H, s); ESIMS found for C₁₇H₁₆F3N7 m/z 376.0 (M+1).

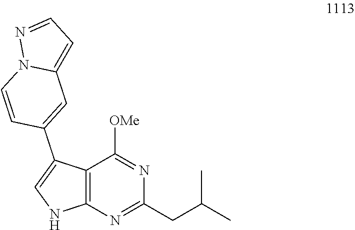 [SMILES: COc1nc(CC(C)C)nc2[nH]cc(-c3ccn4nccc4c3)c12]

2-Isobutyl-4-methoxy-5-(pyrazolo [1,5-a] pyridin-5-yl)-7H-pyrrolo [2,3-d] pyrimidine 1113.

White solid (44.87 mg, 0.140 mmol). ¹H NMR (400 MHz, DMSO-d₆) δppm 0.95 (6 H, d, J=6.60 Hz), 2.28 (1 H, dquin, J=13 .5 5 , 6.70, 6.70, 6.70, 6.70 Hz), 2.69 (2 H, d, J=7.09 Hz), 4.05 (3 H, s), 6.58 (1 H, d, J=1.10 Hz), 7.24 (1 H, dd, J=7.21, 1.34 Hz), 7.75 (1 H, s), 7.96 (1 H, d, J=1.71 Hz), 8.04 (1 H, s), 8.63 (1 H, d, J=7.21 Hz), 12.22 (1 H, br s); ESIMS found for C₁₈H₁₉N50 m/z 322.1 (M+1).

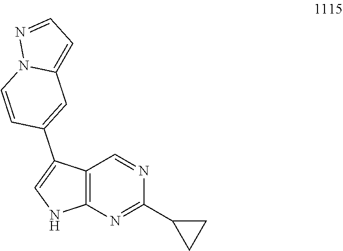 [SMILES: c1cc2cc(-c3c[nH]c4nc(C5CC5)ncc34)ccn2n1]

2—Cyclopropyl-5-(pyrazolo[1,5-a]pyridin-5-yl)-7H-pyrrolo[2,3-d]pyrimidine 1115.

Off-white solid (29 mg, 0.105 mmol, 38.8% yield). ¹1-1 NMR (499 MHz, DMSO-d₆) δppm 0.99-1.07 (4 H, m), 2.20-2.31 (1 H, m), 6.55-6.63 (1 H, m), 7.31 (1 H, dd,

J=7.26, 2.05 Hz), 7.97 (1 H, d, J=2.19 Hz), 8.07 (1 H, s), 8.08 (1 H, d, J=1.64 Hz), 8.67 (1 H, d,

J=7.39 Hz), 9.35 (1 H, s), 12.23 (1 H, br s); ESIMS found for C₁₆H₁₃N₅ m/z 276.1 (M+1).

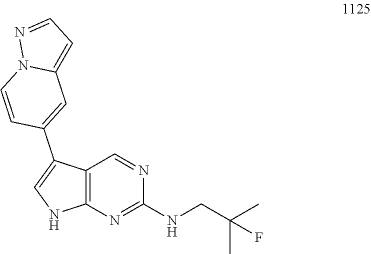 [SMILES: CC(C)(F)CNc1ncc2c(-c3ccn4nccc4c3)c[nH]c2n1]

N-(2-Fluoro-2-methylpropyl)-5-(pyrazolo [1,5-a]pyridin-5-yl)-7H-pyrrolo [2,3-d]pyrimidin-2-amine 1125.

White solid (31.7 mg, 0.098 mmol). ¹H NMR (400 MHz, DMSO-d₆) δppm 1.35 (6 H, d, J=21.40 Hz), 3.59 (2 H, dd, J=18.95, 6.48 Hz), 6.56 (1 H, d, J=1.59 Hz), 6.93 (1 H, br t, J=6.36 Hz), 7.25 (1 H, dd, J=7.27, 1.90 Hz), 7.70 (1 H, d, J=2.45 Hz), 7.94 (1 H, d, J=2.20 Hz), 7.98 (1 H, d, J=1.10 Hz), 8.62 (1 H, d, J=7.34 Hz), 9.05 (1 H, s), 11.63 (1 H, br s); ESIMS found for C₁₇H₁₇FN6 m/z 325.0 (M+1).

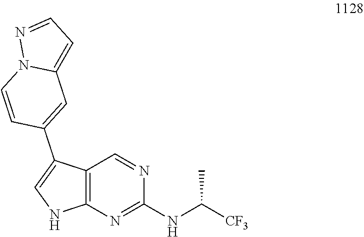 [SMILES: C[C@@H](Nc1ncc2c(-c3ccn4nccc4c3)c[nH]c2n1)C(F)(F)F]

(R)-5-(Pyrazolo [1,5-a]pyridin-5-yl)-N-(1,1,1-trifluoropropan-2-yl)-7H-pyrrolo [2,3-d] pyrimidin-2-amine 1128.

White solid (38.3 mg, 0.111 mmol). ¹H NMR (400 MHz, DMSO-d₆) δppm 1.36 (3 H, d, J=7.09 Hz), 4.86-5.04 (1 H, m), 6.57 (1 H, d, J=1.71 Hz), 7.26 (1 H, dd, J=7.34, 1.96 Hz), 7.44 (1 H, br d, J=8.80 Hz), 7.77 (1 H, d, J=2.20 Hz), 7.95 (1 H, d, J=2.08 Hz), 8.01 (1 H, d, J=1.34 Hz), 8.63 (1 H, d, J=7.34 Hz), 9.11 (1 H, s), 11.77 (1 H, br s); ESIMS found for C₁₆H₁₃F₃N₆ m/z 347.1 (M+1).

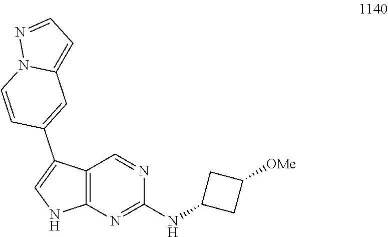 [SMILES: CO[C@H]1C[C@@H](Nc2ncc3c(-c4ccn5nccc5c4)c[nH]c3n2)C1]

N-(cis-3-Methoxycyclobutyl)-5-(pyrazolo[1,5-a]pyridin-5-yl)-7H-pyrrolo [2,3-d]pyrimidin-2-amine 1140.

White solid (8.45 mg, 0.025 mmol). ¹H NMR (400 MHz, DMSO-d₆) δppm 1.79-1.92 (2 H, m), 2.57-2.70 (2 H, m), 3.14 (3 H, s), 3.57-3.66 (1 H, m), 3.90-4.08 (1 H, m), 6.55 (1 H, d, J=1.75 Hz), 7.12 (1 H, br d, J=7 .7 5 Hz), 7.24 (1 H, dd, J=7.38, 2.00 Hz), 7.68 (1 H, s), 7.93 (1 H, d, J=2.13 Hz), 7.96 (1 H, d, J=1.25 Hz), 8.61 (1 H, d, J=7.38 Hz), 9.02 (1 H, s), 11.59 (1 H, s); ESIMS found for C₁₈H₁₈N₆O m/z 335.1 (M+1).

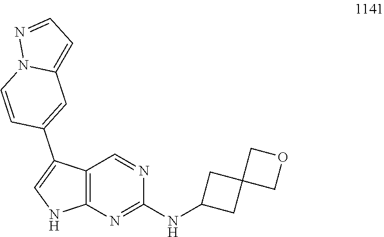 [SMILES: c1cc2cc(-c3c[nH]c4nc(NC5CC6(COC6)C5)ncc34)ccn2n1]

5-(Pyrazolo [1,5-a]pyridin-5-yl)-N-(2-oxaspiro [3 .3]heptan-6-yl)-7H-pyrrolo [2,3-d]pyrimidin-2-amine 1141.

White solid (13.86 mg, 0.040 mmol). ¹H NMR (400 MHz, DMSO-d₆) δppm 2.08-2.21 (2 H, m), 2.60 (2 H, ddd, J=9.81, 7.43, 2.81 Hz), 4.18 (1 H, sxt, J=7.78 Hz), 4.51 (2 H, s), 4.64 (2 H, s), 6.49-6.58 (1 H, m), 7.09 (1 H, br d, J=6.97 Hz), 7.24 (1 H, dd, J=7.46, 1.96 Hz), 7.68 (1 H, s), 7.93 (1 H, d, J=2.20 Hz), 7.95 (1 H, d, J=1.22 Hz), 8.60 (1 H, d, J=7.34 Hz), 9.02 (1 H, s); ESIMS found for C₁₉H₁₈N60 m/z 347.2 (M+1).

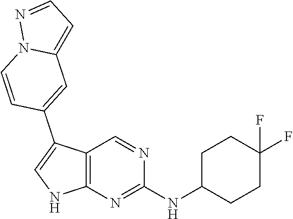 [SMILES: FC1(F)CCC(Nc2ncc3c(-c4ccn5nccc5c4)c[nH]c3n2)CC1]

N-(4,4-Difluorocyclohexyl)-5-(pyrazolo[1,5-a]pyridin-5-yl)-7H-pyrrolo[2,3-d]pyrimidin-2-amine 1142.

Beige solid (18 mg, 0.049 mmol, 23.9% yield). ¹H NMR (499 MHz, DMSO-d₆) δppm 1.56-1.70 (2 H, m), 1.85-2.01 (4 H, m), 2.04-2.16 (2 H, m), 3.94 (1 H, br d, J=8.21 Hz), 6.53-6.58 (1 H, m), 6.92 (1 H, br d, J=7.39 Hz), 7.25 (1 H, dd, J=7.26, 2.05 Hz), 7.69 (1 H, d, J=2.46 Hz), 7.94 (1 H, d, J=2.19 Hz), 7.98 (1 H, d, J=1.64 Hz), 8.62 (1 H, d, J=7.39 Hz), 9.05 (1 H, s), 11.64 (1 H, br s); ESIMS found for C₁₉H₁₈F₂N₆ m/z 369.2 (M+1).

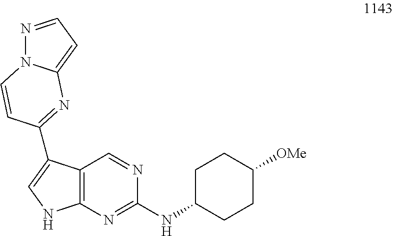 [SMILES: CO[C@H]1CC[C@@H](Nc2ncc3c(-c4ccn5nccc5n4)c[nH]c3n2)CC1]

N-(cis-4-Methoxycyclohexyl)-5-(pyrazolo [1,5-a]pyridin-5-yl)-7H-pyrrolo [2,3-d]pyrimidin-2-amine 1143.

White solid (26.68 mg, 0.074 mmol). ¹H NMR (400 MHz, DMSO-d₆) δppm 1.44-1.53 (2 H, m), 1.53-1.64 (2 H, m), 1.64-1.73 (2 H, m), 1.79-1.90 (2 H, m), 3.22 (3 H, s), 3.34 (1 H, br s), 3.73-3.85 (1 H, m), 6.54 (1 H, d, J=1.59 Hz), 6.64 (1 H, br d, J=7.70 Hz), 7.23 (1 H, dd, J=7.34, 1.71 Hz), 7.66 (1 H, s), 7.93 (1 H, d, J=2.20 Hz), 7.94 (1 H, br s), 8.60 (1 H, d, J=7.34 Hz), 9.01 (1 H, s); ESIMS found for C₂₀1⁻1₂₂N₆O m/z 363.2 (M+1).

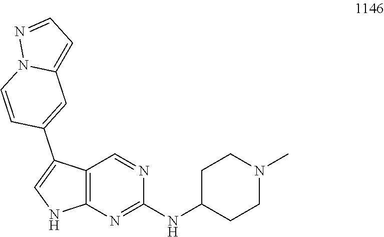 [SMILES: CN1CCC(Nc2ncc3c(-c4ccn5nccc5c4)c[nH]c3n2)CC1]

N-(1-Methylpiperidin-4-yl)-5-(pyrazolo [1,5-a] pyridin-5-yl)-7H-pyrrolo [2,3-d] pyrimidin-2-amine 1146.

White solid (7.19 mg, 0.021 mmol). ¹1-1 NMR (400 MHz, DMSO-d₆) δppm 1.45-1.62 (2 H, m), 1.83-1.91 (2 H, m), 1.95 (2 H, br t, J=11.51 Hz), 2.17 (3 H, s), 2.76 (2 H, br d, J=11.51 Hz), 3.64-3.77 (1 H, m), 6.55 (1 H, d, J=1.88 Hz), 6.69 (1 H, br d, J=7.63 Hz), 7.24(1 H, dd, J=7.38, 1.75 Hz), 7.66 (1 H, s), 7.93 (1 H, d, J=2.13 Hz), 7.96 (1 H, s), 8.60 (1 H, d, J=7.38 Hz), 9.03 (1 H, s), 11.58 (1 H, br s); ESIMS found for C₁₉H_{2I}N₇ m/z 348.2 (M+1).

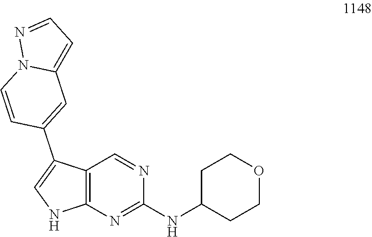 [SMILES: c1cc2cc(-c3c[nH]c4nc(NC5CCOCC5)ncc34)ccn2n1]

5-(Pyrazolo [1,5-a]pyridin-5-yl)-N-(tetrahydro-2H-pyran-4-yl)-7H-pyrrolo [2,3-d]pyrimidin-2-amine 1148.

White solid (39.63 mg, 0.118 mmol). ¹H NMR (400 MHz, DMSO-d₆) δppm 1.45-1.61 (2 H, m), 1.88 (2 H, br dd, J=12.41, 2.14 Hz), 3.39 (2 H, td, J=11.65, 2.02 Hz), 3.83-3.93 (2 H, m), 3.93-4.04 (1 H, m), 6.56 (1 H, dd, J=2.20, 0.61 Hz), 6.85 (1 H, br d, J=7.82 Hz), 7.24 (1 H, dd, J=7.34, 1.96 Hz), 7.68 (1 H, d, J=2.32 Hz), 7.94 (1 H, d, J=2.20 Hz), 7.97 (1 H, d, J=1.34 Hz), 8.61 (1 H, d, J=7.34 Hz), 9.05 (1 H, s), 11.64 (1 H, br s); ESIMS found for C₁₈14₁₈1\1₆0 m/z 335.2 (M+1).

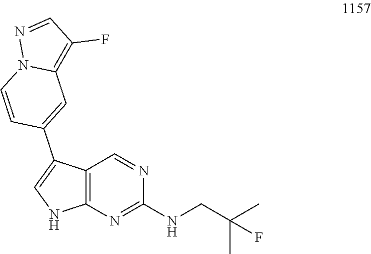 [SMILES: CC(C)(F)CNc1ncc2c(-c3ccn4ncc(F)c4c3)c[nH]c2n1]

N-(2-Fluoro-2-methylpropyl)-5-(3-fluoropyrazolo [1,5-a] pyridin-5-yl)-7H-pyrrolo [2,3-d]pyrimidin-2-amine 1157.

White solid (2 mg, 0.006 mmol, 56.4% yield). ¹H NMR (499 MHz, DMSO-d₆) δppm 1.35 (6 H, d, J=21.40 Hz), 3.59 (2 H, dd, J=19.16, 6.57 Hz), 6.95 (1 H, br s), 7.27 (1 H, dd, J=7.67, 2.19 Hz), 7.75 (1 H, s), 7.82 (1 H, d, J=1.64 Hz), 8.05 (1 H, d, J=3.29 Hz), 8.53 (1 H, d, J=7.12 Hz), 9.03 (1 H, s), 11.68 (1 H, br s); ESIMS found for C₁₇H₁₆F₂N₆ m/z 343.1 (M+1).

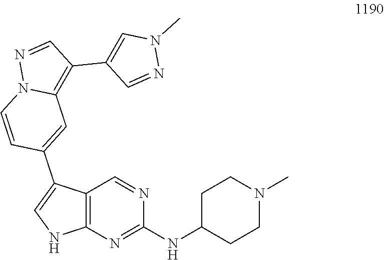 [SMILES: CN1CCC(Nc2ncc3c(-c4ccn5ncc(-c6cnn(C)c6)c5c4)c[nH]c3n2)CC1]

5-(3-(1-Methyl-1H-pyrazol-4-yl)pyrazolo [1,5-a] pyridin-5-yl)-N-(1-methylpiperidin-4-yl)-7H-pyrrolo [2,3-d]pyrimidin-2-amine 1190.

Yellow solid (5.4 mg, 0.013 mmol, 9.2% yield). ¹H NMR (499 MHz, DMSO-d₆) δppm 1.49-1.59 (2 H, m), 1.88 (2 H, br d, J=9.58 Hz), 1.92-2.00 (2 H, m), 2.17 (3 H, s), 2.76 (2 H, br d, J=11.50 Hz), 3.65-3.77 (1 H, m), 3.91 (3 H, s), 6.68 (1 H, br d, J=7.94 Hz), 7.24 (1 H, dd, J=7.39, 1.92 Hz), 7.73 (1 H, s), 7.85-7.92 (2 H, m), 8.17 (1 H, s), 8.19 (1 H, s), 8.62 (1 H, dd, J=7.39, 0.82 Hz), 8.95 (1 H, s), 11.66 (1 H, br s); ESIMS found for C23H₂₅N9 M/Z 428.15 (M+1).

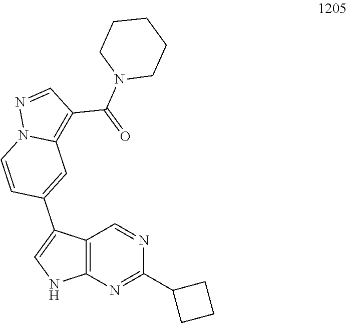 [SMILES: O=C(c1cnn2ccc(-c3c[nH]c4nc(C5CCC5)ncc34)cc12)N1CCCCC1]

(5-(2—Cyclobutyl-7H-pyrrolo[2,3-d]pyrimidin-5-yl)pyrazolo[1,5-a]pyridin-3-yl)(piperidin-1-yl)methanone 1205.

Beige solid (45 mg, 0.112 mmol, 89.0% yield). ¹H NMR (499 MHz, DMSO-d₆) δppm 1.52-1.61 (4 H, m), 1.62-1.70 (2 H, m), 1.84-1.95 (1 H, m), 1.97-2.12 (1 H, m), 2.28 -2.38 (2 H, m), 2.39-2.48 (2 H, m), 3.61-3.69 (4 H, m), 3.77-3.88 (1 H, m), 7.49 (1 H, dd, J=7.26, 2.05 Hz), 8.16 (1 H, d, J=1.37 Hz), 8.21 (1 H, s), 8.22 (1 H, s), 8.76-8.82 (1 H, m), 9.30 (1 H, s), 12.46 (1 H, s); ESIMS found for C23H₂₄N60 m/z 401.2 (M+1).

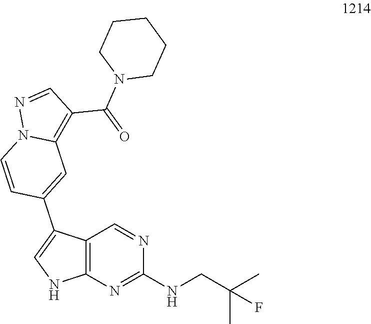 [SMILES: CC(C)(F)CNc1ncc2c(-c3ccn4ncc(C(=O)N5CCCCC5)c4c3)c[nH]c2n1]

(5-(2-((2-Fluoro-2-methylpropyl)amino)-7H-pyrrolo [2,3-d]pyrimidin-5-yl) pyrazolo [1,5-a]pyridin-3-yl)(pip eridin-1-yl)methanone 1214.

Beige solid (17 mg, 0.039 mmol, 46.0% yield). ¹H NMR (499 MHz, DMSO-d₆) δppm 1.35 (6 H, d, J=21.40 Hz), 1.53-1.61 (4 H, m), 1.62-1.71 (2 H, m), 3.60 (2 H, dd, J=19.03, 6.43 Hz), 3.63-3.66 (4 H, m), 6.99 (1 H, br t, J=5.48 Hz), 7.42 (1 H, dd, J=7.39, 1.92 Hz), 7.80 (1 H, s), 8.06 (1 H, d, J=1.37 Hz), 8.18 (1 H, s), 8.73 (1 H, d, J=7.39 Hz), 8.90 (1 H, s), 11.75 (1 H, br s); ESIMS found for C₂₃H₂₆FN₇O m/z 436.2 (M+1).

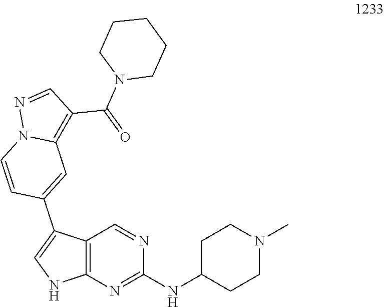 [SMILES: CN1CCC(Nc2ncc3c(-c4ccn5ncc(C(=O)N6CCCCC6)c5c4)c[nH]c3n2)CC1]

(5-(2-((l-Methylpiperidin-4-yl)amino)-7H-pyrrolo [2,3-d]pyrimidin-5-yl) pyrazolo [1,5-a]pyridin-3-yl)(pip eridin-1-yl)methanone 1233.

Tan solid (4 mg, 0.009 mmol, 9.2% yield). ¹H NMR (499 MHz, DMSO-d₆) 6 ppm 1.53-1.65 (6 H, m), 2.22 (3 H, s), 2.38-2.43 (4 H, m), 3.42-3.49 (4 H, m), 3.61-3.67 (4 H, m), 6.88 (1 H, d, J=9.06 Hz), 7.52 (1 H, dd, J=7.41, 1.92 Hz), 7.93 (1 H, dd, J=9.06, 2.74 Hz), 8.15 (1 H, d, J=1.37 Hz), 8.21 (1 H, s), 8.88 (1 H, d, J=1.92 Hz), 8.92 (1 H, d, J=1.92 Hz), 12.53 (1 H, br s); ESIMS found for C₂₅H₃₀N₈O m/z 459.2 (M+1).

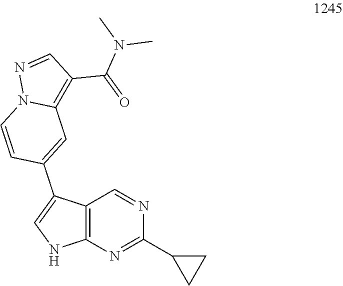 [SMILES: CN(C)C(=O)c1cnn2ccc(-c3c[nH]c4nc(C5CC5)ncc34)cc12]

5-(2—Cyclopropyl-7H-pyrrolo[2,3-d]pyrimidin-5-yl)-N,N-dimethylpyrazolo [1,5-a]pyridine-3-carboxamide 1245.

Tan solid (12 mg, 0.035 mmol, 17.3% yield). ¹H NMR (500 MHz, DMSO-d₆) δppm 1.00-1.09 (4 H, m), 2.21-2.30 (1 H, m), 3.16 (6 H, br s), 7.49 (1 H, dd, J=7.27, 2.06 Hz), 8.16 (1 H, d, J=2.47 Hz), 8.33 (2 H, s), 8.78 (1 H, d, J=7.14 Hz), 9.20 (1 H, s), 12.35 (1 H, br s); ESIMS found for C₁₉H₁₈N60 m/z 347.15 (M+1).

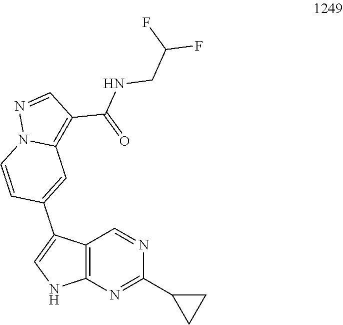 [SMILES: O=C(NCC(F)F)c1cnn2ccc(-c3c[nH]c4nc(C5CC5)ncc34)cc12]

5-(2—Cyclopropyl-7H-pyrrolo[2,3-d]pyrimidin-5-yl)-N-(2,2-difluoroethyl) pyrazolo[1,5-a]pyridine-3-carboxamide 1249.

Beige solid (7 mg, 0.018 mmol, 12.3% yield). ¹H NMR (499 MHz, DMSO-d₆) δppm 1.02-1.08 (4 H, m), 2.22-2.30 (1 H, m), 3.65-3.77 (2 H, m), 6.16 (1 H, tt, J=56.25, 4.10 Hz), 7.52 (1 H, dd, J=7.39, 2.19 Hz), 8.19 (1 H, d, J=2.46 Hz), 8.54 (1 H, d, J=1.92 Hz), 8.58 (1 H, s), 8.58-8.61 (1 H, m), 8.80 (1 H, d, J=7.12 Hz), 9.22 (1 H, s), 12.38 (1 H, br s); ESIMS found for C₁₉H₁₆F₂N₆O m/z 383.1 (M+1).

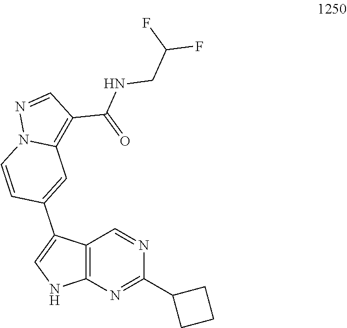 [SMILES: O=C(NCC(F)F)c1cnn2ccc(-c3c[nH]c4nc(C5CCC5)ncc34)cc12]

5-(2—Cyclobutyl-7H-pyrrolo [2,3-d]pyrimidin-5-yl)-N-(2,2-difluoroethyl) pyrazolo[1,5-a]pyridine-3-carboxamide 1250.

White solid (5 mg, 0.013 mmol, 19.8% yield). ¹H NMR (499 MHz, DMSO-d₆) δppm 1.87-1.96 (1 H, m), 2.01-2.12 (1 H, m), 2.29-2.38 (2 H, m), 2.39-2.47(2 H, m), 3.65 -3.78 (2 H, m), 3.79-3.89 (1 H, m), 6.16 (1 H, tt, J=56.20, 4.20 Hz), 7.54 (1 H, dd, J=7.26, 2.05 Hz), 8.25 (1 H, s), 8.56 (1 H, d, J=1.64 Hz), 8.59 (1 H, s), 8.61 (1 H, t, J=6.02 Hz), 8.81 (1 H, s), 9.32 (1 H, s), 12.50 (1 H, br s); ESIMS found for C₂₀H₁₈F2N60 m/z 397.2 (M+1).

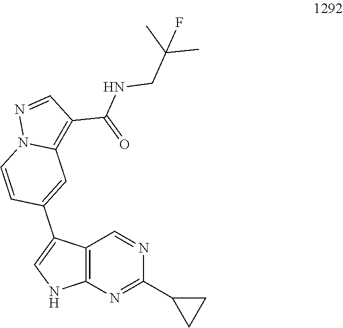 [SMILES: CC(C)(F)CNC(=O)c1cnn2ccc(-c3c[nH]c4nc(C5CC5)ncc34)cc12]

5-(2—Cyclopropyl-7H-pyrrolo[2,3-d]pyrimidin-5-yl)-N-(2-fluoro-2-methylpropyl)pyrazolo[1,5-a]pyridine-3-carboxamide 1292.

Light yellow solid (23 mg, 0.059 mmol, 28.4% yield). ¹H NMR (499 MHz, DMSO-d₆) δppm 1.02-1.07 (4 H, m), 1.36 (6 H, d, J=21.70 Hz), 2.21-2.30 (1 H, m), 3.52 (2 H, dd, J=20.60, 6.30 Hz), 7.50 (1 H, dd, J=7.39, 2.19 Hz), 8.18 (1 H, d, J=1.37 Hz), 8.36 (1 H, t, J=6.30 Hz), 8.55 (1 H, d, J=1.64 Hz), 8.65 (1 H, s), 8.78 (1 H, d, J=7.39 Hz), 9.21 (1 H, s), 12.37 (1 H, s); ESIMS found for C2,H_{2,}FN6O m/z 393.2 (M+1).

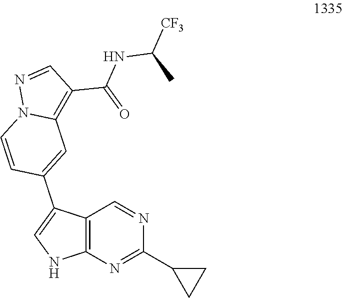 [SMILES: C[C@@H](NC(=O)c1cnn2ccc(-c3c[nH]c4nc(C5CC5)ncc34)cc12)C(F)(F)F]

(R)-5-(2—Cyclopropyl-7H-pyrrolo [2,3-d]pyrimidin-5-yl)-N-(1,1,1-trifluoropropan-2-yl)pyrazolo [1,5-a]pyridine-3-carboxamide 1335.

Tan solid (14 mg, 0.034 mmol, 11.3% yield). ¹H NMR (500 MHz, DMSO-d₆) δppm 1.02-1.08 (4 H, m), 1.39 (3 H, d, J=6.86 Hz), 2.22-2.30 (1 H, m), 4.93 (1 H, dq, J=15.40, 7.95 Hz), 7.54 (1 H, dd, J=7.41, 2.20 Hz), 8.19 (1 H, s), 8.53 (1 H, d, J=1.65 Hz), 8.55 (1 H, d, J=8.78 Hz), 8.66 (1 H, s), 8.80 (1 H, d, J=7.14 Hz), 9.21 (1 H, s), 12.38 (1 H, br s); ESIMS found for C₂₀H₁₇F3N60 m/z 415.2 (M+1).

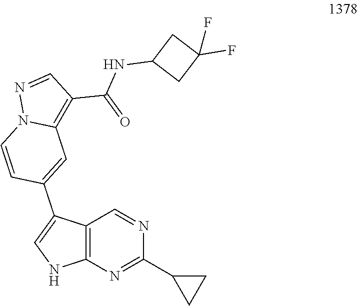 [SMILES: O=C(NC1CC(F)(F)C1)c1cnn2ccc(-c3c[nH]c4nc(C5CC5)ncc34)cc12]

5-(2—Cyclopropyl-7H-pyrrolo [2,3-d] pyrimidin-5-yl)-N-(3,3-difluorocyclobutyppyrazolo [1,5-a] pyridine-3-carboxamide 1378.

White solid (15 mg, 0.037 mmol, 23.3% yield). ¹H NMR (499 MHz, DMSO-d₆) δppm 1.00-1.09 (4 H, m), 2.22-2.31 (1 H, m), 2.68-2.82 (2 H, m), 2.94-3.05 (2 H, m), 4.27 -4.37 (1 H, m), 7.51 (1 H, dd, J=7.39, 2.19 Hz), 8.18 (1 H, s), 8.51-8.55 (3 H, m), 8.78 (1 H, d, J=7.39 Hz), 9.21 (1 H, s), 12.38 (1 H, br s); ESIMS found for C₂iFli₈F₂N₆O m/z 409.2 (M+1).

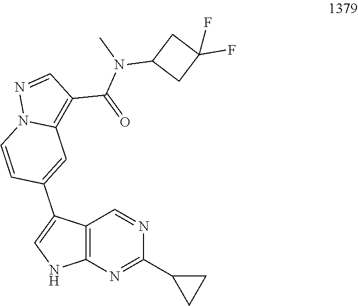 [SMILES: CN(C(=O)c1cnn2ccc(-c3c[nH]c4nc(C5CC5)ncc34)cc12)C1CC(F)(F)C1]

5-(2—Cyclopropyl-7H-pyrrolo [2,3-d] pyrimidin-5-yl)-N-(3,3-difluorocyclobutyl)-N-methylpyrazolo [1,5-a]pyridine-3-carboxamide 1379.

White solid (34 mg, 0.081 mmol, 55.6% yield). ¹H NMR (499 MHz, DMSO-d₆) δppm 1.01-1.06 (4 H, m), 2.21-2.30 (1 H, m), 2.89-3.01 (4 H, m), 3.15 (3 H, s), 4.66-4.77 (1 H, m), 7.52 (1 H, dd, J=7.26, 2.05 Hz), 8.18 (1 H, s), 8.29 (1 H, d, J=1.37 Hz), 8.32 (1 H, s), 8.80 (1 H, d, J=6.57 Hz), 9.20 (1 H, s), 12.37 (1 H, s); ESIMS found for C₂₂H₂₀F₂N₆O m/z 423.2 (M+1).

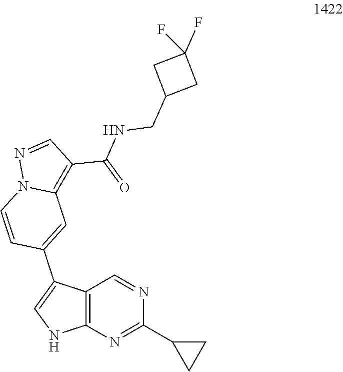 [SMILES: O=C(NCC1CC(F)(F)C1)c1cnn2ccc(-c3c[nH]c4nc(C5CC5)ncc34)cc12]

5-(2—Cyclopropyl-7H-pyrrolo [2,3-d] pyrimidin-5-yl)-N-43 ,3-difluorocyclobutypmethyppyrazolo [1,5-a]pyridine-3-carboxamide 1422.

White solid (5 mg, 0.012 mmol, 7.1% yield). ¹H NMR (499 MHz, DMSO-d₆) δppm 1.01-1.08 (4 H, m), 2.21-2.31 (1 H, m), 2.35-2.47 (3 H, m), 2.61-2.72(2 H, m), 3.42(2 H, br t, J=5.75 Hz), 7.49 (1 H, dd, J=7.26, 2.05 Hz), 8.17 (1 H, s), 8.34 (1 H, t, J=5.89 Hz), 8.51 (1 H, s), 8.52 (1 H, d, J=1.64 Hz), 8.77 (1 H, d, J=7.12 Hz), 9.21 (1 H, s), 12.37 (1 H, s); ESIMS found for C22H₂₀F2N60 m/z 423.2 (M+1).

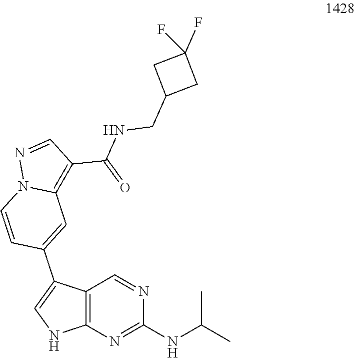 [SMILES: CC(C)Nc1ncc2c(-c3ccn4ncc(C(=O)NCC5CC(F)(F)C5)c4c3)c[nH]c2n1]

N-((3 ,3-Difluorocyclobutypmethyl)-5-(2-(i sopropylamino)-7H-pyrrolo [2,3-d]pyrimidin-5-yl)pyrazolo [1,5-a]pyridine-3-carboxamide 1428.

White solid (13 mg, 0.030 mmol, 36.5% yield). ¹H NMR (499 MHz, DMSO-d₆) δppm 1.19 (6 H, d, J=6.57 Hz), 2.34-2.45 (3 H, m), 2.59-2.73 (2 H, m), 3.41 (2 H, br t, J=5.75 Hz), 4.08 (1 H, dq, J=13.62, 6.78 Hz), 6.74 (1 H, br d, J=7.67 Hz), 7.43 (1 H, dd, J=7.26, 2.05 Hz), 7.79 (1 H, d, J=2.46 Hz), 8.32 (1 H, t, J=5.89 Hz), 8.42 (1 H, d, J=1.64 Hz), 8.48 (1 H, s), 8.71 (1 H, d, J=7.39 Hz), 8.90 (1 H, s), 11.74 (1 H, br s); ESIMS found for C₂₂H₂₃F₂N₇O m/z 440.2 (M+1).

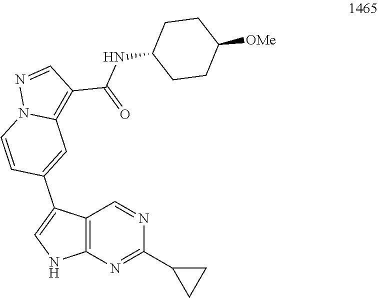 [SMILES: CO[C@H]1CC[C@H](NC(=O)c2cnn3ccc(-c4c[nH]c5nc(C6CC6)ncc45)cc23)CC1]

5-(2—Cyclopropyl-7H-pyrrolo [2,3-d] pyrimidin-5-yl)-N-(trans -4-methoxycyclohexyl)pyrazolo [1,5-a] pyridine-3-carboxamide 1465.

Off-white solid (22 mg, 0.051 mmol, 26.1% yield). ¹H NMR (499 MHz, DMSO-d₆) δppm 1.02-1.07 (4 H, m), 1.16-1.28 (2 H, m), 1.31-1.44 (2 H, m), 1.92 (2 H, br d, J=10.68 Hz), 2.01-2.10 (2 H, m), 2.26 (1 H, tt, J=7 .77 , 4.96 Hz), 3.14 (1 H, tt, J=10.51, 4.14 Hz), 3.26 (3 H, s), 3.81 (1 H, tdt, J=11.36, 11.36, 7.53, 4.04, 4.04 Hz), 7.48 (1 H, dd, J=7.39, 2.19 Hz), 7.95 (1 H, d, J=7.94 Hz), 8.17 (1 H, s), 8.51-8.58 (2 H, m), 8.75 (1 H, d, J=7.39 Hz), 9.21 (1 H, s), 12.36 (1 H, br s); ESIMS found for C24H₂₆N602 m/z 431.2 (M+1).

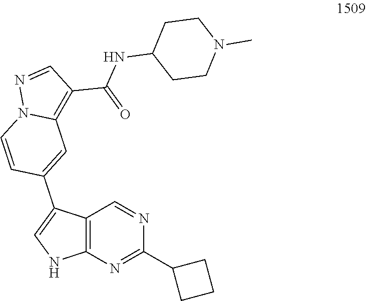 [SMILES: CN1CCC(NC(=O)c2cnn3ccc(-c4c[nH]c5nc(C6CCC6)ncc45)cc23)CC1]

5-(2—Cyclobutyl-7H-pyrrolo [2,3-d]pyrimidin-5-yl)-N-(1-methylpiperidin-4-yl)pyrazolo[1,5-a]pyridine-3-carboxamide 1509.

Beige solid (15 mg, 0.035 mmol, 29.1% yield). ¹H NMR (499 MHz, DMSO-d₆) δppm 1.51-1.65 (2 H, m), 1.82 (2 H, br d, J=10.13 Hz), 1.86-1.92 (1 H, m), 1.93-2.00 (2 H, m), 2.00-2.11 (1 H, m), 2.18 (3 H, s), 2.29-2.39 (2 H, m), 2.40-2.48 (2 H, m), 2.80(2 H, br d, J=11.77 Hz), 3.74-3.82 (1 H, m), 3.81-3.87 (1 H, m), 7.50 (1 H, dd, J=7.26, 2.05 Hz), 7.98 (1 H, d, J=7.94 Hz), 8.22 (1 H, s), 8.57 (2 H, s), 8.77 (1 H, d, J=7.12 Hz), 9.31 (1 H, s), 12.46 (1 H, br s); ESIMS found for C₂₄H₂₇N₇O m/z 430.2 (M+1).

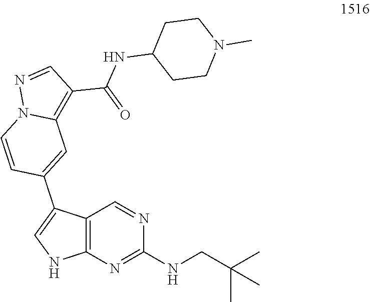 [SMILES: CN1CCC(NC(=O)c2cnn3ccc(-c4c[nH]c5nc(NCC(C)(C)C)ncc45)cc23)CC1]

N-(1-Methylpiperidin-4-yl)-5-(2-(ne opentylamino)-7H-pyrrolo [2,3-d] pyrimidin-5-yl)pyrazolo[1,5-a]pyridine-3-carboxamide 1516.

Off-white solid (25 mg, 0.054 mmol, 55.6% yield). ¹H NMR (499 MHz, DMSO-d₆) δppm 0.93 (9 H, s), 1.57 (2 H, qd, J=12.00, 3.70 Hz), 1.77-1.85 (2 H, m), 1.90-2.01 (2 H, m), 2.17 (3 H, s), 2.79 (2 H, br d, J=11.77 Hz), 3.23 (2 H, d, J=6.30 Hz), 3.73-3.86 (1 H, m), 6.85 (1 H, br s), 7.41 (1 H, dd, J=7.26, 2.05 Hz), 7.77 (1 H, d, J=1.92 Hz), 7.94 (1 H, d, J=7.94 Hz), 8.43 (1 H, d, J=1.37 Hz), 8.53 (1 H, s), 8.67-8.73 (1 H, m), 8.88 (1 H, s), 11.69 (1 H, br s); ESIMS found for C25H₃₂N80 m/z 461.3 (M+1).

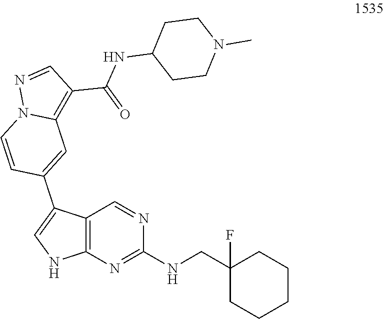 [SMILES: CN1CCC(NC(=O)c2cnn3ccc(-c4c[nH]c5nc(NCC6(F)CCCCC6)ncc45)cc23)CC1]

5-(2-(((l-Fluorocyclohexyl)methyl)amino)-7H-pyrrolo[2,3-d]pyrimidin-5-yl)-N-(1-methylpiperidin-4-yl)pyrazolo[1,5-a]pyridine-3-carboxamide 1535.

Beige solid (15 mg, 0.030 mmol, 39.2% yield). ¹H NMR (499 MHz, DMSO-d₆) δppm 1.24-1.32 (1 H, m), 1.47-1.62 (9 H, m), 1.73-1.86 (4 H, m), 1.91-2.01 (2 H, m), 2.18 (3 H, s), 2.79 (2 H, br d, J=11.77 Hz), 3.56-3.67 (2 H, m), 3.73-3.85 (1 H, m), 6.94(1 H, br s), 7.42 (1 H, dd, J=7.39, 2.19 Hz), 7.80 (1 H, s), 7.95 (1 H, d, J=7.94 Hz), 8.45 (1 H, d, J=1.37 Hz), 8.54 (1 H, s), 8.69 (1 H, d, J=7.12 Hz), 8.91 (1 H, s), 11.74 (1 H, br s); ESIMS found for C27H₃₃FN₈O m/z 505.3 (M+1).

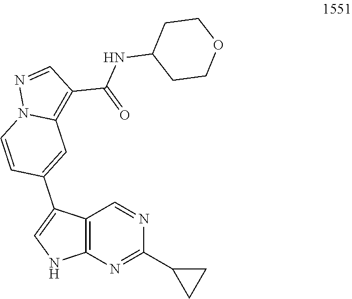 [SMILES: O=C(NC1CCOCC1)c1cnn2ccc(-c3c[nH]c4nc(C5CC5)ncc34)cc12]

5-(2—Cyclopropyl-7H-pyrrolo[2,3-d]pyrimidin-5-yl)-N-(tetrahydro-2H-pyran-4-yl)pyrazolo[1,5-a]pyridine-3-carboxamide 1551.

Off-white solid (6 mg, 0.015 mmol, 15.9% yield). ¹1-1 NMR (499 MHz, DMSO-d₆) δppm 1.02-1.07 (4 H, m), 1.58 (2 H, qd, J=11 .9 6 , 4.38 Hz), 1.82 (2 H, br dd, J=12.46, 2.33 Hz), 2.22-2.30 (1 H, m), 3.40 (2 H, td, J=11.77, 1.64 Hz), 3.86-3.95 (2 H, m), 4.00-4.11 (1 H, m), 7.49 (1 H, dd, J=7.26, 2.05 Hz), 8.04 (1 H, d, J=7.67 Hz), 8.17 (1 H, s), 8.55 (1 H, d, J=1.64 Hz), 8.56 (1 H, s), 8.76 (1 H, d, J=7.39 Hz), 9.21 (1 H, s), 12.37 (1 H, br s); ESIMS found for C22H₂₂N602 m/z 403.2 (M+1).

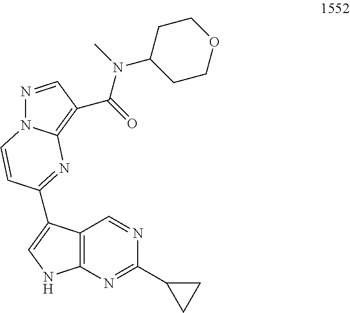 [SMILES: CN(C(=O)c1cnn2ccc(-c3c[nH]c4nc(C5CC5)ncc34)nc12)C1CCOCC1]

5-(2—Cyclopropyl-7H-pyrrolo[2,3-d]pyrimidin-5-yl)-N-methyl-N-(tetrahydro-2H-pyran-4-yl)pyrazolo[1,5-a]pyridine-3-carboxamide 1552.

Off-white solid (6 mg, 0.014 mmol, 15.8% yield). ¹1-1 NMR (499 MHz, DMSO-d₆) δppm 1.00-1.06 (4 H, m), 1.65 (2 H, br dd, J=12.18, 2.05 Hz), 1.86 (2 H, qd, J=12.23, 4.93 Hz), 2.20-2.31 (1 H, m), 3.07 (3 H, s), 3.42 (2 H, br t, J=11.36 Hz), 3.94 (2 H, dd, J=11 .09 , 4.24 Hz), 4.48-4.61 (1 H, m), 7.50 (1 H, dd, J=7.39, 2.19 Hz), 8.17 (1 H, s), 8.31 (2 H, br d, J=12.05 Hz), 8.79 (1 H, dd, J=7.12, 0.82 Hz), 9.19 (1 H, s), 12.36 (1 H, br s); ESIMS found for

C₂₃H₂₄N₆O₂ m/z 417.2 (M+1).

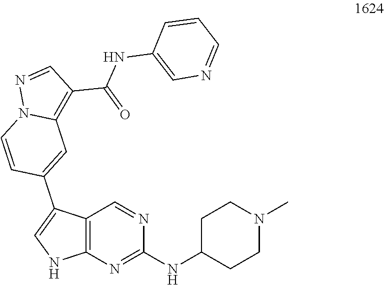 [SMILES: CN1CCC(Nc2ncc3c(-c4ccn5ncc(C(=O)Nc6cccnc6)c5c4)c[nH]c3n2)CC1]

5-(2-((1-Methylpiperidin-4-yl)amino)-7H-pyrrolo[2,3-d]pyrimidin-5-yl)-N-(pyridin-3-yl)pyrazolo[1,5-a]pyridine-3-carboxamide 1624.

Off-white solid (2.5 mg, 0.005 mmol, 5.4% yield). ¹1-1 NMR (499 MHz, DMSO-d₆) δppm 1.48-1.63 (2 H, m), 1.88 (2 H, br d, J=10.13 Hz), 1.92-2.00 (2 H, m), 2.17 (3 H, s), 2.76 (2 H, br d, J=11.77 Hz), 3.66-3.79 (1 H, m), 6.80 (1 H, br d, J=6.02 Hz), 7.39 (1 H, dd, J=8.49, 4.65 Hz), 7.51 (1 H, dd, J=7.26, 2.05 Hz), 7.84 (1 H, s), 8.25 (1 H, dd, J=8.35, 2.05 Hz), 8.29 (1 H, dd, J=4.65, 1.10 Hz), 8.48 (1 H, d, J=1.37 Hz), 8.76 (1 H, s), 8.79 (1 H, d, J=7.12 Hz), 8.92 (1 H, d, J=2.46 Hz), 8.93 (1 H, s), 10.13 (1 H, s), 11.80 (1 H, br s); ESIMS found for C25H₂₅N90 m/z 468.1 (M+1).

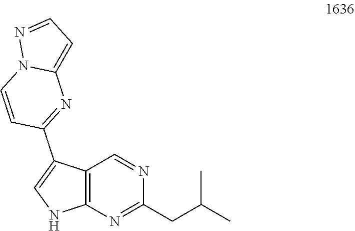 [SMILES: CC(C)Cc1ncc2c(-c3ccn4nccc4n3)c[nH]c2n1]

2-Isobutyl-5-(pyrazolo[1,5-a]pyrimidin-5-yl)-7H-pyrrolo[2,3-d]pyrimidine 1636.

Off-white solid (26 mg, 0.089 mmol, 30.6% yield). ¹1-1 NMR (499 MHz, DMSO-d₆) δppm 0.93 (6 H, d, J=6.57 Hz), 2.28 (1 H, dquin, J=13.52, 6.82, 6.82, 6.82, 6.82 Hz), 2.81 (2 H, d, J=7.12 Hz), 6.66 (1 H, d, J=2.19 Hz), 7.57 (1 H, d, J=7.39 Hz), 8.14 (1 H, d, J=2.19 Hz), 8.55 (1 H, s), 9.05 (1 H, d, J=7.39 Hz), 9.73 (1 H, s), 12.52 (1 H, br s); ESIMS found for C₁₆H₁₆N6 m/z 293.15 (M+1).

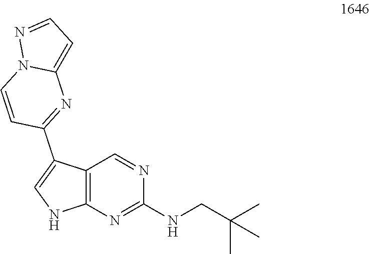 [SMILES: CC(C)(C)CNc1ncc2c(-c3ccn4nccc4n3)c[nH]c2n1]

N-Ne opentyl-5-(pyrazolo [1,5-a]pyrimidin-5-yl)-7H-pyrrolo [2,3-d] pyrimidin-2-amine 1646.

White solid (23.11 mg, 0.072 mmol). ¹H NMR (400 MHz, DMSO-d₆) δppm 0.93 (9 H, s), 3.22 (2 H, br d, J=6.25 Hz), 6.59 (1 H, d, J=1.88 Hz), 6.80 (1 H, br t, J=5.57 Hz), 7.46 (1 H, d, J=7.38 Hz), 8.09 (1 H, d, J=2.25 Hz), 8.10 (1 H, s), 8.93 (1 H, d, J=7.50 Hz), 9.30 (1 H, s); ESIMS found for C₁₇H₁₉N₇ m/z 322.2 (M+1).

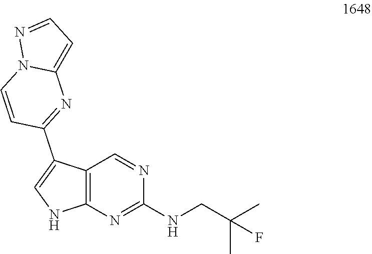 [SMILES: CC(C)(F)CNc1ncc2c(-c3ccn4nccc4n3)c[nH]c2n1]

N-(2-Fluoro-2-methylpropyl)-5-(pyrazolo [1,5-a]pyrimidin-5-yl)-7H-pyrrolo [2,3-d]pyrimidin-2-amine 1648.

Beige solid (2.18 g, 6.701 mmol, 95.7% yield). ¹H NMR (499 MHz, DMSO-d₆) δppm 1.35 (6 H, d, J=21.40 Hz), 3.59 (2 H, dd, J=18.62, 6.57 Hz), 6.61 (1 H, d, J=1.64 Hz), 7.00 (1 H, br s), 7.48 (1 H, d, J=7.12 Hz), 8.10 (1 H, d, J=2.74 Hz), 8.14 (1 H, s), 8.96 (1 H, d, J=7.67 Hz), 9.34 (1 H, s), 11.87 (1 H, br s); ESIMS found for C₁₆H₁₆FN₇ m/z 326.2 (M+1).

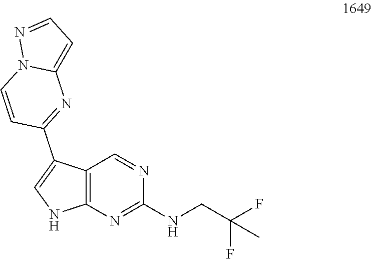 [SMILES: CC(F)(F)CNc1ncc2c(-c3ccn4nccc4n3)c[nH]c2n1]

N-(2,2-Difluoropropyl)-5-(pyrazolo[1,5-a]pyrimidin-5-yl)-7H-pyrrolo[2,3-d] pyrimidin-2-amine 1649.

White solid (5.19 mg, 0.016 mmol). ¹H NMR (400 MHz, DMSO-d₆) δppm 1.63 (3 H, t, J=19.01 Hz), 3.83 (2 H, td, J=13.54, 6.44 Hz), 6.61 (1 H, d, J=1.50 Hz), 7.30 (1 H, br t, J=5.50 Hz), 7.48 (1 H, d, J=7.50 Hz), 8.11 (1 H, d, J=2.13 Hz), 8.18 (1 H, s), 8.97 (1 H, d, J=7.38 Hz), 9.37 (1 H, s), 11.91 (1 H, br s); ESIMS found for CisH₁₃F₂N₇ m/z 330.1 (M+1).

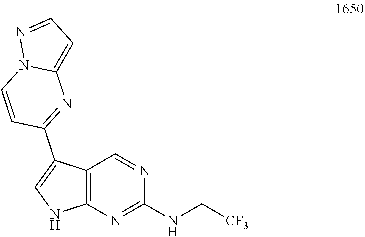 [SMILES: FC(F)(F)CNc1ncc2c(-c3ccn4nccc4n3)c[nH]c2n1]

5-(Pyrazolo [1,5-a]pyrimidin-5-yl)-N-(2,2,2-trifluoroethyl)-7H-pyrrolo [2,3-d] pyrimidin-2-amine 1650.

White solid (4.86 mg, 0.015 mmol ¹H NMR (400 MHz, DMSO-d₆) δppm 4.09-4.26 (2 H, m), 6.59-6.67 (1 H, m), 7.49 (1 H, d, J=7.38 Hz), 7.57 (1 H, br t, J=6.82 Hz), 8.11 (1 H, d, J=2.13 Hz), 8.22 (1 H, s), 8.98 (1 H, d, J=7.38 Hz), 9.41 (1 H, s), 12.05 (1 H, br s); ESIMS found for CHHI0F3N7 m/z 334.1 (M+1).

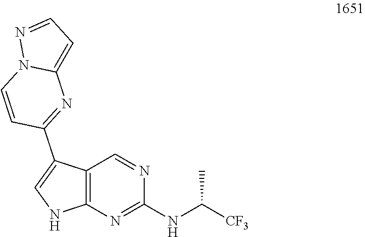 [SMILES: C[C@@H](Nc1ncc2c(-c3ccn4nccc4n3)c[nH]c2n1)C(F)(F)F]

(R)-5-(Pyrazolo [1,5-a]pyrimidin-5-yl)-N-(1,1,1-trifluoropropan-2-yl)-7H-pyrrolo [2,3-d] pyrimidin-2-amine 1651.

White solid (29.5 mg, 0.085 mmol). ¹H NMR (400 MHz, DMSO-d₆) δppm 1.36 (3 H, d, J=7.00 Hz), 4.86-5.02 (1 H, m), 6.62 (1 H, dd, J=2.19, 0.81 Hz), 7.48-7.54 (2 H, m), 8.11 (1 H, d, J=2.25 Hz), 8.21 (1 H, s), 8.98 (1 H, dd, J=7.44, 0.81 Hz), 9.40 (1 H, s), 12.01 (1 H, br s); ESIMS found for CisH₁₂F₃N₇ m/z 348.1 (M+1).

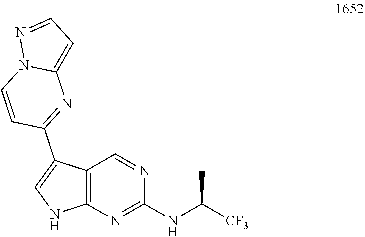 [SMILES: C[C@H](Nc1ncc2c(-c3ccn4nccc4n3)c[nH]c2n1)C(F)(F)F]

(S)-5-(Pyrazolo[1,5-a]pyrimidin-5-yl)-N-(1,1,1-trifluoropropan-2-yl)-7H-pyrrolo[2,3-d]pyrimidin-2-amine 1652.

White solid (17.72 mg, 0.051 mmol). ¹H NMR (400 MHz, DMSO-d₆) δppm 1.43 (3 H, d, J=7.00 Hz), 4.98 (1 H, br dd, J=14.26, 7.13 Hz), 6.67 (1 H, d, J=1.75 Hz), 7.56 (1 H, d, J=7.38 Hz), 8.16 (1 H, d, J=2.25 Hz), 8.53 (1 H, d, J=2.13 Hz), 8.69 (1 H, br s), 9.07 (1 H, d, J=7.50 Hz), 9.43 (1 H, s), 12.87 (1 H, br s); ESIMS found for CisH₁₂F₃N₇ m/z 348.0 (M +1).

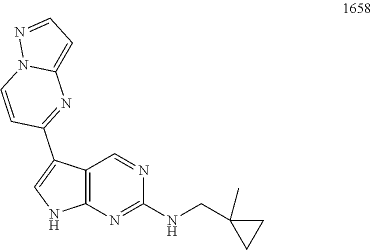 [SMILES: CC1(CNc2ncc3c(-c4ccn5nccc5n4)c[nH]c3n2)CC1]

N-((l-Methylcyclopropyl)methyl)-5-(pyrazolo[1,5-a]pyrimidin-5-yl)-7H-pyrrolo [2,3-d] pyrimidin-2-amine 1658.

White solid (26.16 mg, 0.082 mmol). ¹H NMR (400 MHz, DMSO-d₆) δppm 0.17-0.27 (2 H, m), 0.45-0.54 (2 H, m), 1.09 (3 H, s), 3.29 (2 H, d, J=5.88 Hz), 6.60 (1 H, d, J=1.75 Hz), 6.88-6.94 (1 H, m), 7.47 (1 H, d, J=7.38 Hz), 8.10 (1 H, d, J=2.25 Hz), 8.11 (1 H, s), 8.95 (1 H, d, J=7.50 Hz), 9.31 (1 H, s), 11.83 (1 H, br s); ESIMS found for C₁₇H₁₇N₇ m/z 320.2 (M+1).

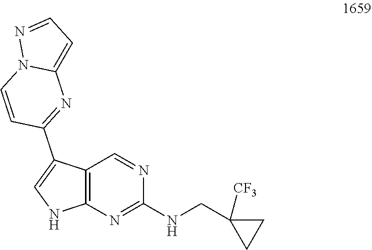 [SMILES: FC(F)(F)C1(CNc2ncc3c(-c4ccn5nccc5n4)c[nH]c3n2)CC1]

5-(Pyrazolo[1,5-a]pyrimidin-5-yl)-N-41-(trifluoromethyl)cyclopropyl) methyl)-7H-pyrrolo [2,3-d]pyrimidin-2-amine 1659.

White solid (26.93 mg, 0.072 mmol). ¹H NMR (400 MHz, DMSO-d₆) δppm 0.81-0.89 (2 H, m), 0.94 (2 H, br s), 3.75 (2 H, d, J=6.50 Hz), 6.58 (1 H, d, J=2.00 Hz), 6.94 (1 H, br t, J=5.13 Hz), 7.45 (1 H, d, J=7.38 Hz), 8.08 (1 H, d, J=2.13 Hz), 8.14 (1 H, s), 8.92 (1 H, d, J=7.50 Hz), 9.32 (1 H, s); ESIMS found for C₁₇H₁₄F₃N₇ m/z 374.0 (M+1).

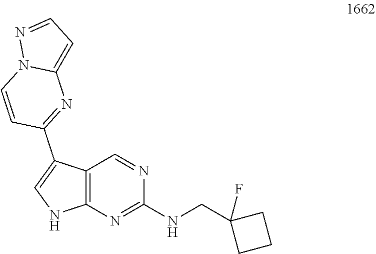 [SMILES: FC1(CNc2ncc3c(-c4ccn5nccc5n4)c[nH]c3n2)CCC1]

N-((l-Fluorocyclobutyl)methyl)-5-(pyrazolo [1,5-a]pyrimidin-5-yl)-7H-pyrrolo [2,3-d] pyrimidin-2-amine 1662.

White solid (15.23 mg, 0.045 mmol). ¹H NMR (400 MHz, DMSO-d₆) δppm 1.47-1.64 (1 H, m), 1.71-1.84 (1 H, m), 2.10-2.32 (4 H, m), 3.72 (2 H, dd, J=22.70, 6.30 Hz), 6.52 (1 H, d, J=1.71 Hz), 6.74 (1 H, br s), 7.41 (1 H, d, J=7.58 Hz), 8.05 (1 H, d, J=2.20 Hz), 8.12 (1 H, s), 8.86 (1 H, d, J=7.34 Hz), 9.27 (1 H, s); ESIMS found for C₁₇H₁₆FN₇ m/z 338.2 (M+1).

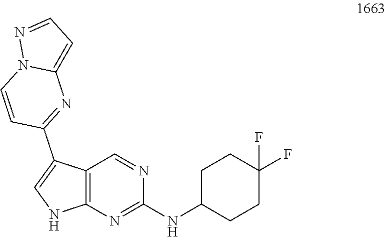 [SMILES: FC1(F)CCC(Nc2ncc3c(-c4ccn5nccc5n4)c[nH]c3n2)CC1]

N-(4,4-Difluorocyclohexyl)-5-(pyrazolo [1,5-a]pyrimidin-5-yl)-7H-pyrrolo [2,3-d]pyrimidin-2-amine 1663.

White solid (28.29 mg, 0.077 mmol). ¹H NMR (400 MHz, DMSO-d₆) δppm 1.56-1.73 (2 H, m), 1.81-2.03 (4 H, m), 2.04-2.19 (2 H, m), 3.86-4.01 (1 H, m), 6.61 (1 H, dd, J=2.26, 0.79 Hz), 6.97-7.08 (1 H, m), 7.48 (1 H, d, J=7.46 Hz), 8.11 (1 H, d, J=2.32 Hz), 8.14 (1 H, s), 8.96 (1 H, dd, J=7.40, 0.79 Hz), 9.35 (1 H, s), 11.90 (1 H, br s); ESIMS found for C₁₈1-1₁₇F₂N₇ m/z 370.2 (M+1).

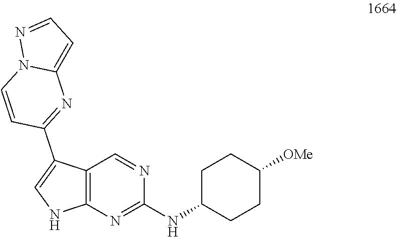 [SMILES: CO[C@H]1CC[C@@H](Nc2ncc3c(-c4ccn5nccc5n4)c[nH]c3n2)CC1]

N-(cis-4-Methoxycyclohexyl)-5-(pyrazolo [1,5-a]pyrimidin-5-yl)-7H-pyrrolo [2,3-d] pyrimidin-2-amine 1664.

White solid (27.78 mg, 0.077 mmol). ¹H NMR (400 MHz, DMSO-d₆) δppm 1.44-1.54 (2 H, m), 1.54-1.65 (2 H, m), 1.66-1.73 (2 H, m), 1.81-1.91 (2 H, m), 3.23 (3 H, s), 3.33-3.43 (2 H, m), 3.74-3.87 (1 H, m), 6.59 (1 H, d, J=2.00 Hz), 6.79 (1 H, br d, J=7.50 Hz), 7.46 (1 H, d, J=7.38 Hz), 8.04-8.13 (2 H, m), 8.95 (1 H, d, J=7.50 Hz), 9.31 (1 H, s), 11.84 (1 H, br s); ESIMS found for C₁₉H₂iN70 m/z 364.1 (M+1).

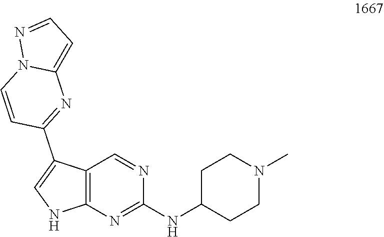 [SMILES: CN1CCC(Nc2ncc3c(-c4ccn5nccc5n4)c[nH]c3n2)CC1]

N-(1-Methylpiperidin-4-yl)-5-(pyrazolo [1,5-a] pyrimidin-5-yl)-7H-pyrrolo [2,3-d]pyrimidin-2-amine 1667.

White solid (54.11 mg, 0.155 mmol). ¹H NMR (400 MHz, DMSO-d₆) δppm 1.53 (2 H, qd, J=11.59, 3.38 Hz), 1.88 (2 H, br d, J=11.13 Hz), 1.95 (2 H, br t, J=11.51 Hz), 2.16 (3 H, s), 2.75 (2 H, br d, J=11.51 Hz), 3.65-3.77 (1 H, m), 6.58 (1 H, d, J=2.00 Hz), 6.75 (1 H, br d, J=7.63 Hz), 7.45 (1 H, d, J=7.38 Hz), 8.09 (1 H, d, J=2.25 Hz), 8.10 (1 H, s), 8.92 (1 H, d, J=7.50 Hz), 9.31 (1 H, s); ESIMS found for C₁₈H₂₀N8 m/z 349.1 (M+1).

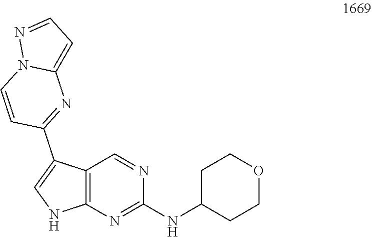 [SMILES: c1cc2nc(-c3c[nH]c4nc(NC5CCOCC5)ncc34)ccn2n1]

5-(Pyrazolo [1,5-a] pyrimidin-5-yl)-N-(tetrahydro-2H-pyran-4-yl)-7H-pyrrolo [2,3-d]pyrimidin-2-amine 1669.

White solid (18.73 mg, 0.056 mmol). ¹H NMR (400 MHz, DMSO-d₆) δppm 1.44-1.63 (2 H, m), 1.88 (2 H, br d, J=12.01 Hz), 3.40 (2 H, br t, J=11.13 Hz), 3.89 (2 H, br d, J=11.38 Hz), 3.95 (1 H, br d, J=7.38 Hz), 6.58 (1 H, s), 6.86 (1 H, br d, J=6.13 Hz), 7.45 (1 H, br d, J=7.38 Hz), 8.08 (1 H, br s), 8.11 (1 H, s), 8.92 (1 H, br d, J=7.25 Hz), 9.32 (1 H, s); ESIMS found for C₁₇H₁₇N₇O m/z 336.1 (M+1).

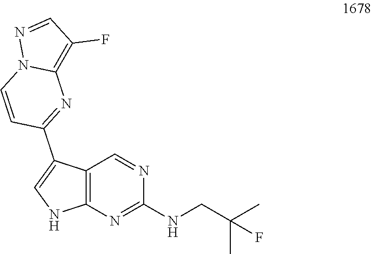 [SMILES: CC(C)(F)CNc1ncc2c(-c3ccn4ncc(F)c4n3)c[nH]c2n1]

N-(2-Fluoro-2-methylpropyl)-5-(3-fluoropyrazolo [1,5-a] pyrimidin-5-yl)-7H-pyrrolo [2,3-d] pyrimidin-2-amine 1678.

White solid (4.77 mg, 0.014 mmol). ¹H NMR (400 MHz, DMSO-d₆) δppm 1.35 (6 H, d, J=21.40 Hz), 3.59 (2 H, br dd, J=18.95, 6.85 Hz), 7.00-7.10 (1 H, m), 7.49 (1 H, d,

J=7.58 Hz), 8.20 (1 H, s), 8.24 (1 H, d, J=3.42 Hz), 8.89 (1 H, dd, J=7.52, 1.53 Hz), 9.31 (1 H, s), 11.95 (1 H, br s); ESIMS found for C₁₆H₁₅F₂N₇ m/z 344.1 (M+1).

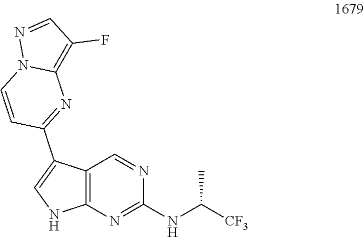 [SMILES: C[C@@H](Nc1ncc2c(-c3ccn4ncc(F)c4n3)c[nH]c2n1)C(F)(F)F]

(R)-5-(3-Fluoropyrazolo [1,5-a]pyrimidin-5-yl)-N-(1,1,1-trifluoropropan-2-yl)-7H-pyrrolo [2,3-d] pyrimidin-2-amine 1679.

White solid (12.98 mg, 0.036 mmol). ¹H NMR (400 MHz, DMSO-d₆) δppm 1.36 (3 H, d, J=7.13 Hz), 4.94 (1 H, br dd, J=13.45, 6.69 Hz), 7.51 (1 H, d, J=7.50 Hz), 7.55 (1 H, br d, J=8.00 Hz), 8.25 (1 H, d, J=3.50 Hz), 8.26 (1 H, s), 8.90 (1 H, d, J=7.50 Hz), 9.36 (1 H, s), 12.06 (1 H, br s); ESIMS found for CisH₁ₗF₄N₇ m/z 366.1 (M+1).

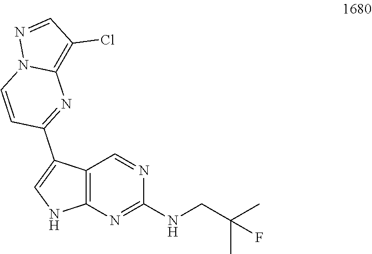 [SMILES: CC(C)(F)CNc1ncc2c(-c3ccn4ncc(Cl)c4n3)c[nH]c2n1]

5-(3-Chloropyrazolo [1,5-a] pyrimidin-5-yl)-N-(2-fluoro-2-methylpropyl)-7H-pyrrolo [2,3-d] pyrimidin-2-amine 1680.

White solid (39.75 mg, 0.111 mmol). ¹H NMR (400 MHz, DMSO-d₆) δppm 1.35 (6 H, d, J=21.40 Hz), 3.60 (2 H, br dd, J=19.26, 6.42 Hz), 6.94 (1 H, br t, J=6.36 Hz), 7.54 (1 H, d, J=7.46 Hz), 8.22 (1 H, s), 8.23 (1 H, s), 8.95 (1 H, d, J=7.46 Hz), 9.38 (1 H, s); ESIMS found for C₁₆H₁₅ClFN7 m/z 360.1 (M+1).

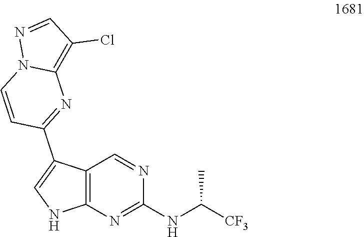 [SMILES: C[C@@H](Nc1ncc2c(-c3ccn4ncc(Cl)c4n3)c[nH]c2n1)C(F)(F)F]

(R)-5-(3-Chloropyrazolo [1,5-a]pyrimidin-5-yl)-N-(1,1,1-trifluoropropan-2-yl)-7H-pyrrolo [2,3-d] pyrimidin-2-amine 1681.

White solid (75.24 mg, 0.197 mmol). ¹H NMR (400 MHz, DMSO-d₆) δppm 1.37 (3 H, d, J=7.00 Hz), 4.97 (1 H, dq, J=15.07, 7.57 Hz), 7.45 (1 H, br d, J=9.01 Hz), 7.56 (1 H, d, J=7.50 Hz), 8.24 (1 H, s), 8.29 (1 H, s), 8.97 (1 H, d, J=7.38 Hz), 9.43 (1 H, s), 12.09 (1 H, br s); ESIMS found for CistliiClF3N7 m/z 382.1 (M+1).

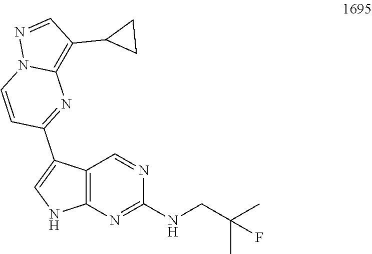 [SMILES: CC(C)(F)CNc1ncc2c(-c3ccn4ncc(C5CC5)c4n3)c[nH]c2n1]

5-(3 —Cyclopropylpyrazolo [1,5-a] pyrimidin-5-yl)-N-(2-fluoro-2-methylpropyl)-7H-pyrrolo [2,3-d] pyrimidin-2-amine 1695.

White solid (14.07 mg, 0.039 mmol). ¹H NMR (400 MHz, DMSO-d₆) δppm 0.93-1.02 (4 H, m), 1.35 (6 H, d, J=21.40 Hz), 2.03-2.15 (1 H, m), 3.60 (2 H, br dd, J=19.20, 6.32 Hz), 6.94 (1 H, br t, J=5.50 Hz), 7.38 (1 H, d, J=7.50 Hz), 7.90 (1 H, s), 8.11 (1 H, s), 8.84 (1 H, d, J=7.50 Hz), 9.36 (1 H, s), 11.84 (1 H, br s); ESIMS found for C₁₉H₂₀FN₇ m/z 366.1 (M+1).

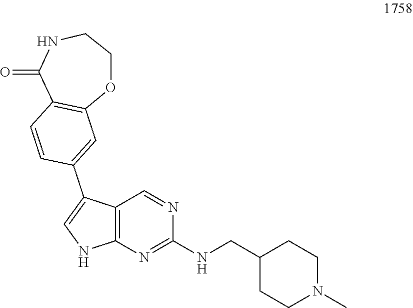 [SMILES: CN1CCC(CNc2ncc3c(-c4ccc5c(c4)OCCNC5=O)c[nH]c3n2)CC1]

8-(2-(((l-Methylpiperidin-4-yl)methyl)amino)-7H-pyrrolo [2,3-d]pyrimidin-5-yl)-3,4-dihydrobenzo[f][1,4]oxazepin-5(2H)-one 1758.

Off-white solid (9 mg, 0.022 mmol, 40.0% yield). ¹1-1 NMR (499 MHz, DMSO-d₆) δppm 1.17 (2 H, qd, J=12.08, 3.57 Hz), 1.51-1.62 (1 H, m), 1.67 (2 H, br d, J=10.98 Hz), 1.78 (2 H, td, J=11.60, 2.06 Hz), 2.12 (3 H, s), 2.69-2.77 (2 H, m), 3.18 (2 H, t, J=6.45 Hz), 3.34-3.37 (2 H, m), 4.30-4.36 (2 H, m), 6.87 (1 H, br s), 7.28 (1 H, d, J=1.92 Hz), 7.46 (1 H, dd, J=8.23, 1.65 Hz), 7.57 (1 H, s), 7.82 (1 H, d, J=8.23 Hz), 8.25 (1 H, t, J=5.21 Hz), 8.85 (1 H, s), 11.57 (1 H, br s); ESIMS found for C₂₂H₂₆N₆O₂ m/z 407.1 (M+1).

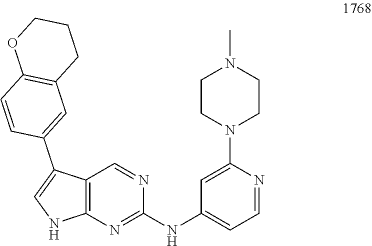 [SMILES: CN1CCN(c2cc(Nc3ncc4c(-c5ccc6c(c5)CCCO6)c[nH]c4n3)ccn2)CC1]

5—(Chroman-6-yl)-N-(2-(4-methylpiperazin-1-yl)pyridin-4-yl)-7H-pyrrolo [2,3-d]pyrimidin-2-amine 1768.

Beige solid (10 mg, 0.023 mmol, 9.4% yield). ¹H NMR (500 MHz, DMSO-d₆) δppm 1.89-1.99 (2 H, m), 2.22 (3 H, s), 2.37-2.45 (4 H, m), 2.82 (2 H, t, J=6.31 Hz), 3.36-3.44 (4 H, m), 4.11-4.19 (2 H, m), 6.77 (1 H, d, J=9.06 Hz), 6.81 (1 H, d, J=9.06 Hz), 7.34-7.43 (3 H, m), 7.95 (1 H, dd, J=9.06, 2.74 Hz), 8.55 (1 H, d, J=2.47 Hz), 8.93 (1 H, s), 9.05 (1 H, s), 11.51 (1 H, s); ESIMS found for C₂₅H₂₇N₇O m/z 442.1 (M+1).

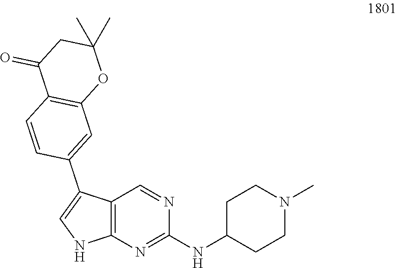 [SMILES: CN1CCC(Nc2ncc3c(-c4ccc5c(c4)OC(C)(C)CC5=O)c[nH]c3n2)CC1]

2,2-Dimethyl-7-(2-((1-methylpipe ridin-4-yl)amino)-7H-pyrrolo [2,3-d] pyrimidin-5-yl)chroman-4-one 1801.

Yellowish white solid (4 mg, 0.010 mmol, 11.5% yield). ¹H NMR (499 MHz, DMSO-d₆) δppm 1.42 (6 H, s), 1.48-1.56 (2 H, m), 1.82-1.90 (2 H, m), 1.95 (2 H, br t, J=10.84 Hz), 2.16 (3 H, s), 2.75 (2 H, br d, J=12.08 Hz), 2.77 (2 H, s), 3.63-3.75 (1 H, m), 6.68-6.79 (1 H, m), 7.25 (1 H, d, J=1.37 Hz), 7.39 (1 H, dd, J=8.23, 1.65 Hz), 7.67 (1 H, d, J=1.92 Hz), 7.73 (1 H, d, J=8.23 Hz), 8.88 (1 H, s), 11.69 (1 H, br s); ESIMS found for C₂₃H₂₇N₅O₂ m/z 406.1 (M+1).

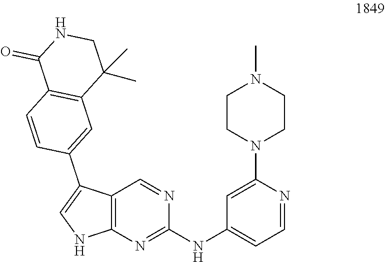 [SMILES: CN1CCN(c2cc(Nc3ncc4c(-c5ccc6c(c5)C(C)(C)CNC6=O)c[nH]c4n3)ccn2)CC1]

4,4-Dimethyl-6-(2-42-(4-methylpiperazin-1-yl)pyridin-4-yl)amino)-7H-pyrrolo [2,3-d]pyrimidin-5-yl)-3,4-dihydroisoquinolin-1(2H)-one 1849.

Off-white solid (17 mg, 0.035 mmol, 23.6% yield). ¹H NMR (500 MHz, DMSO-d₆) δppm 1.36 (6 H, s), 2.23 (3 H, s), 2.39-2.46 (4 H, m), 3.20 (2 H, d, J=2.74 Hz), 3.43-3.50 (4 H, m), 7.16 (1 H, dd, J=5.76, 1.65 Hz), 7.48 (1 H, d, J=1.65 Hz), 7.70 (1 H, d, J=1.65 Hz), 7.74 (1 H, dd, J=7.96, 1.65 Hz), 7.87-7.94 (4 H, m), 9.13 (1 H, s), 9.63 (1 H, s), 12.06 (1 H, br s); ESIMS found for C₂₇H₃₀1\1₈0 m/z 483.2 (M+1).

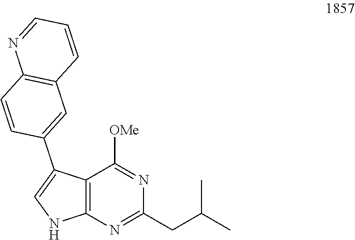 [SMILES: COc1nc(CC(C)C)nc2[nH]cc(-c3ccc4ncccc4c3)c12]

6-(2-Isobutyl-4-methoxy-7H-pyrrolo[2,3-d]pyrimidin-5-yl)quinoline 1857.

White solid (9.6 mg, 0.029 mmol). ¹H NMR (400 MHz, DMSO-d₆) δppm 0.95 (6 H, d, J=6.60 Hz), 2.29 (1 H, dquin, J=13.57, 6.79, 6.79, 6.79, 6.79 Hz), 2.70 (2 H, d, J=7.21 Hz), 4.03 (3 H, s), 7.52 (1 H, dd, J=8.25, 4.22 Hz), 7.70 (1 H, s), 8.00 (1 H, d, J=8.80 Hz), 8.11 (1 H, dd, J=8.74, 2.02 Hz), 8.28 (1 H, d, J=1.96 Hz), 8.35 (1 H, dd, J=8.44, 1.10 Hz), 8.85 (1 H, dd, J=4.16, 1.71 Hz), 12.19 (1 H, br s); ESIMS found for C₂₀H₂₀N₄O m/z 333.0 (M+1).

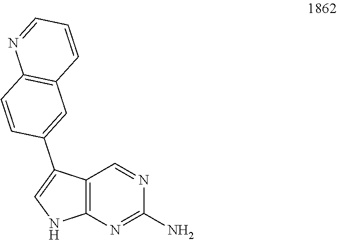 [SMILES: Nc1ncc2c(-c3ccc4ncccc4c3)c[nH]c2n1]

5-(Quinolin-6-yl)-7H-pyrrolo[2,3-d]pyrimidin-2-amine 1862.

Beige solid (17 mg, 0.065 mmol, 13.9% yield). ¹H NMR (499 MHz, DMSO-d₆) δppm 6.24 (2 H, s), 7.51 (1 H, dd, J=8.23, 4.12 Hz), 7.69 (1 H, s), 8.00 (1 H, d, J=8.78 Hz), 8.14 (1 H, dd, J=8.78, 1.92 Hz), 8.28 (1 H, d, J=1.65 Hz), 8.44 (1 H, d, J=7.68 Hz), 8.82 (1 H, dd, J=4.12, 1.65 Hz), 9.09 (1 H, s), 11.50 (1 H, br s); ESIMS found for CisHiiN₅ m/z 262.1 (M+1).

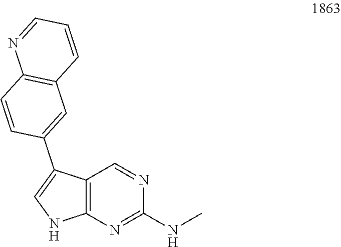 [SMILES: CNc1ncc2c(-c3ccc4ncccc4c3)c[nH]c2n1]

N-Methyl-5-(quinolin-6-yl)-7H-pyrrolo[2,3-d]pyrimidin-2-amine 1863.

Beige solid (5 mg, 0.018 mmol, 39.0% yield). ¹H NMR (499 MHz, DMSO-d₆) δppm 2.85 (3 H, d, J=4.94 Hz), 6.77 (1 H, br d, J=4.39 Hz), 7.51 (1 H, dd, J=8.37, 4.25 Hz), 7.68 (1 H, d, J=1.65 Hz), 8.00 (1 H, d, J=8.78 Hz), 8.14 (1 H, dd, J=8.78, 1.92 Hz), 8.28 (1 H, d, J=1.92 Hz), 8.44 (1 H, d, J=7.41 Hz), 8.82 (1 H, dd, J=4.12, 1.65 Hz), 9.13 (1 H, s), 11.61 (1 H, br s); ESIMS found for C₁₆H₁₃N₅ m/z 276.1 (M+1).

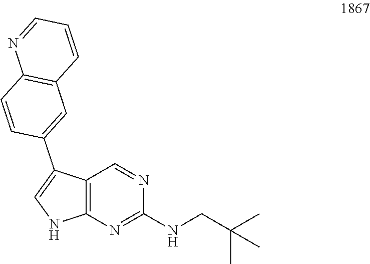 [SMILES: CC(C)(C)CNc1ncc2c(-c3ccc4ncccc4c3)c[nH]c2n1]

N-Ne opentyl-5-(quinolin-6-yl)-7H-pyrrolo [2,3-d] pyrimidin-2-amine 1867.

White solid (6.84 mg, 0.021 mmol). ¹H NMR (400 MHz, DMSO-d₆) δppm 0.93 (9 H, s), 3.24 (2 H, br d, J=6.36 Hz), 6.72 (1 H, br t, J=6.05 Hz), 7.51 (1 H, dd, J=8.25, 4.22 Hz), 7.66 (1 H, s), 7.99 (1 H, d, J=8.93 Hz), 8.14 (1 H, dd, J=8.80, 1.96 Hz), 8.27 (1 H, d, J=1.71 Hz), 8.44 (1 H, d, J=7.58 Hz), 8.81 (1 H, dd, J=4.16, 1.59 Hz), 9.11 (1 H, s), 11.52 (1 H, br s); ESIMS found for C₂₀H₂,N₅ m/z 332.2 (M+1).

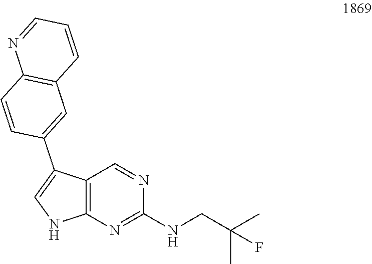 [SMILES: CC(C)(F)CNc1ncc2c(-c3ccc4ncccc4c3)c[nH]c2n1]

N-(2-Fluoro-2-methylpropyl)-5-(quinolin-6-yl)-7H-pyrrolo [2,3-d]pyrimidin-2-amine 1869.

White solid (3.6 mg, 0.011 mmol). ¹H NMR (400 MHz, DMSO-d₆) δppm 1.36 (6 H, d, J=21.40 Hz), 3.61 (2 H, dd, J=18.70, 6.44 Hz), 6.90 (1 H, br t, J=6.32 Hz), 7.52 (1 H, dd, J=8.44, 4.06 Hz), 7.70 (1 H, d, J=2.25 Hz), 8.00 (1 H, d, J=8.88 Hz), 8.15 (1 H, dd, J=8.82, 2.19 Hz), 8.29 (1 H, d, J=1.75 Hz), 8.45 (1 H, s), 8.82 (1 H, dd, J=4.13, 1.75 Hz), 9.15 (1 H, s), 11.61 (1 H, br s); ESIMS found for C₁₉HisFN₅ m/z 336.0 (M+1).

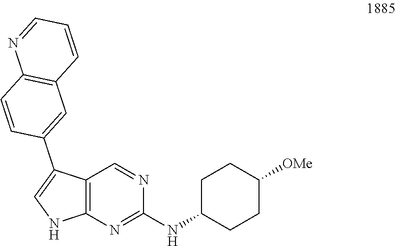 [SMILES: CO[C@H]1CC[C@@H](Nc2ncc3c(-c4ccc5ncccc5c4)c[nH]c3n2)CC1]

N-(cis-4-Methoxycyclohexyl)-5-(quinolin-6-yl)-7H-pyrrolo [2,3-d] pyrimidin-2-amine 1885.

White solid (13.50 mg, 0.036 mmol). ¹H NMR (400 MHz, DMSO-d₆) δppm 1.43-1.54 (2 H, m), 1.54-1.65 (2 H, m), 1.66-1.76 (2 H, m), 1.80-1.93 (2 H, m), 3.23 (3 H, s), 3.37-3.41 (1 H, m), 3.75-3.89 (1 H, m), 6.68 (1 H, br d, J=7.38 Hz), 7.51 (1 H, dd, J=8.32, 4.19 Hz), 7.66 (1 H, s), 7.99 (1 H, d, J=8.88 Hz), 8.14 (1 H, dd, J=8.88, 1.88 Hz), 8.27 (1 H, d, J=1.63 Hz), 8.43 (1 H, d, J=7 .7 5 Hz), 8.81 (1 H, dd, J=4.13, 1.63 Hz), 9.12 (1 H, s), 11.57 (1 H, br s); ESIMS found for C22H₂₃N50 m/z 374.2 (M+1).

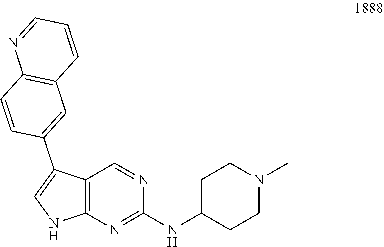 [SMILES: CN1CCC(Nc2ncc3c(-c4ccc5ncccc5c4)c[nH]c3n2)CC1]

N-(1-Methylpiperidin-4-yl)-5-(quinolin-6-yl)-7H-pyrrolo[2,3-d]pyrimidin-2-amine 1888.

White solid (7 mg, 0.020 mmol, 25.7% yield). ¹H NMR (499 MHz, DMSO-d₆) δppm 1.54 (2 H, qd, J=11.66, 3.43 Hz), 1.86-1.92 (2 H, m), 1.96 (2 H, br t, J=11.53 Hz), 2.17 (3 H, s), 2.76 (2 H, br d, J=11.53 Hz), 3.66-3.78 (1 H, m), 6.69 (1 H, br d, J=7.41 Hz), 7.51 (1 H, dd, J=8.23, 4.39 Hz), 7.67 (1 H, s), 7.99 (1 H, d, J=8.78 Hz), 8.14 (1 H, dd, J=8.78, 1.92 Hz), 8.28 (1 H, d, J=1.92 Hz), 8.44 (1 H, d, J=7.41 Hz), 8.81 (1 H, dd, J=4.12, 1.65 Hz), 9.13 (1 H, s), 11.59 (1 H, br s); ESIMS found for C₂₁1-1₂₂N₆ m/z 359.2 (M+1).

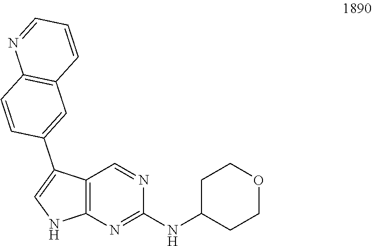 [SMILES: c1cnc2ccc(-c3c[nH]c4nc(NC5CCOCC5)ncc34)cc2c1]

5-(Quinolin-6-yl)-N-(tetrahydro-2H-pyran-4-yl)-7H-pyrrolo [2,3-d] pyrimidin-2-amine 1890.

White solid (24.43 mg, 0.071 mmol). ¹H NMR (400 MHz, DMSO-d₆) δppm 1.46-1.63 (2 H, m), 1.89 (2 H, br dd, J=12.59, 2.32 Hz), 3.38-3.46 (2 H, m), 3.87-3.93 (2 H, m), 3.93-4.03 (1 H, m), 6.81 (1 H, br d, J=7.95 Hz), 7.51 (1 H, dd, J=8.31, 4.16 Hz), 7.68 (1 H, s), 7.99 (1 H, d, J=8.80 Hz), 8.14 (1 H, dd, J=8.80, 2.08 Hz), 8.28 (1 H, d, J=1.96 Hz), 8.44 (1 H, dd, J=8.50, 1.04 Hz), 8.81 (1 H, dd, J=4.28, 1.71 Hz), 9.14 (1 H, s), 11.59 (1 H, br s); ESIMS found for C20F119N50 m/z 346.1 (M+1).

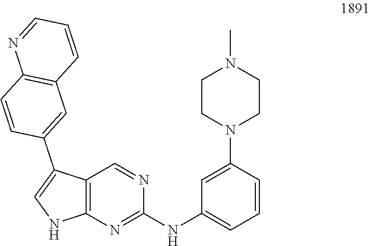 [SMILES: CN1CCN(c2cccc(Nc3ncc4c(-c5ccc6ncccc6c5)c[nH]c4n3)c2)CC1]

N-(3-(4-Methylpipe razin-1-yl)phenyl)-5-(quinolin-6-yl)-7H-pyrrolo [2,3-d] pyrimidin-2-amine 1891.

Beige solid (10 mg, 0.023 mmol, 54.2% yield). ¹H NMR (499 MHz, DMSO-d₆) δppm 2.24 (3 H, s), 2.45-2.49 (4 H, m), 3.11-3.19 (4 H, m), 6.52 (1 H, dd, J=8.23, 1.92 Hz), 7.10 (1 H, t, J=8.10 Hz), 7.33-7.41 (1 H, m), 7.53 (1 H, dd, J=8.23, 4.12 Hz), 7.56 (1 H, s), 7.87 (1 H, s), 8.03 (1 H, d, J=8.78 Hz), 8.20 (1 H, dd, J=8.92, 2.06 Hz), 8.37 (1 H, d, J=1.92 Hz), 8.47 (1 H, d, J=7.96 Hz), 8.83 (1 H, dd, J=4.12, 1.65 Hz), 9.26 (1 H, s), 9.31 (1 H, s), 11.89 (1 H, br s); ESIMS found for C26H₂₅N7 m/z 436.2 (M+1).

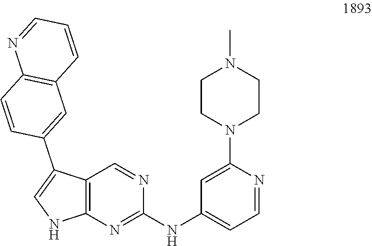 [SMILES: CN1CCN(c2cc(Nc3ncc4c(-c5ccc6ncccc6c5)c[nH]c4n3)ccn2)CC1]

N-(2-(4-Methylpiperazin-1-yl)pyridin-4-yl)-5-(quinolin-6-yl)-7H-pyrrolo [2,3-d]pyrimidin-2-amine 1893.

Beige solid (32 mg, 0.073 mmol, 27.4% yield). ¹H NMR (499 MHz, DMSO-d₆) δppm 2.24 (3 H, s), 2.43 (4 H, br t, J=4.94 Hz), 3.44-3.50 (4 H, m), 7.15 (1 H, dd, J=5.63, 1.51 Hz), 7.52-7.56 (2 H, m), 7.92 (1 H, d, J=5.76 Hz), 7.97 (1 H, s), 8.04 (1 H, d, J=8.78 Hz), 8.22 (1 H, dd, J=8.78, 1.92 Hz), 8.39 (1 H, d, J=1.92 Hz), 8.44-8.53 (1 H, m), 8.84 (1 H, dd, J=4.25, 1.78 Hz), 9.39 (1 H, s), 9.68 (1 H, s), 12.07 (1 H, br s); ESIMS found for C25H₂₄N8 MiZ 437.2 (M+1).

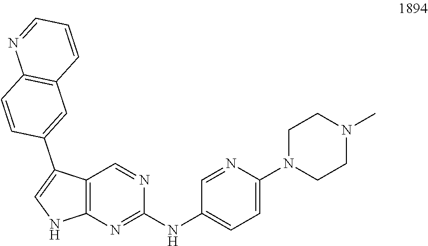 [SMILES: CN1CCN(c2ccc(Nc3ncc4c(-c5ccc6ncccc6c5)c[nH]c4n3)cn2)CC1]

N-(6-(4-Methylpipe razin-1-yl)pyridin-3-yl)-5-(quinolin-6-yl)-7H-pyrrolo [2,3-d]pyrimidin-2-amine 1894.

Beige solid (6.0 mg, 0.014 mmol, 4.8% yield). ¹H NMR (499 MHz, DMSO-d₆) δppm 2.22 (3 H, s), 2.39-2.45 (4 H, m), 3.37-3.44 (4 H, m), 6.83 (1 H, d, J=9.06 Hz), 7.53 (1 H, dd, J=8.23, 4.12 Hz), 7.81 (1 H, d, J=1.10 Hz), 7.98 (1 H, dd, J=9.06, 2.74 Hz), 8.02 (1 H, d, J=8.78 Hz), 8.18 (1 H, dd, J=8.78, 2.20 Hz), 8.34 (1 H, d, J=1.92 Hz), 8.46 (1 H, d, J=7.41 Hz), 8.57 (1 H, d, J=2.47 Hz), 8.83 (1 H, dd, J=4.25, 1.78 Hz), 9.16 (1 H, s), 9.27(1 H, s), 11.82(1 H, br s); ESIMS found for C₂₅H₂₄N₈ M/Z 437.2 (M+1).

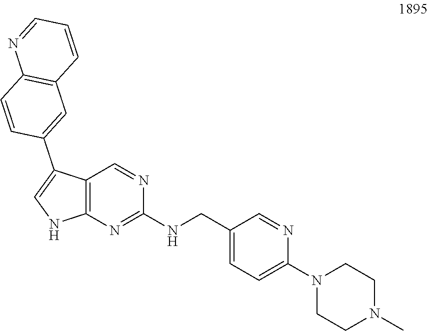 [SMILES: CN1CCN(c2ccc(CNc3ncc4c(-c5ccc6ncccc6c5)c[nH]c4n3)cn2)CC1]

N-((6-(4-Methylpiperazin-1-yl)pyridin-3-yl)methyl)-5-(quinolin-6-yl)-7H-pyrrolo [2,3-d] pyrimidin-2-amine 1895.

Beige solid (5 mg, 0.011 mmol, 4.8% yield). ¹H NMR (499 MHz, DMSO-d₆) δppm 2.19 (3 H, s), 2.36 (4 H, t, J=4.94 Hz), 3.38-3.45 (4 H, m), 4.41 (2 H, br d, J=6.04 Hz), 6.77 (1 H, d, J=8.78 Hz), 7.29 (1 H, t, J=6.10 Hz), 7.51 (1 H, dd, J=8.37, 3.98 Hz), 7.56 (1 H, dd, J=8.92, 2.33 Hz), 7.69 (1 H, s), 7.99 (1 H, d, J=8.78 Hz), 8.10-8.18 (2 H, m), 8.28 (1 H, d, J=1.37 Hz), 8.44 (1 H, d, J=7.68 Hz), 8.81 (1 H, dd, J=3.98, 1.51 Hz), 9.14 (1 H, s), 11.60 (1 H, br s); ESIMS found for C26H₂₆N8 M/Z 451.2 (M+1).

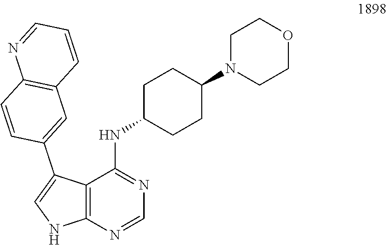 [SMILES: c1cnc2ccc(-c3c[nH]c4ncnc(N[C@H]5CC[C@H](N6CCOCC6)CC5)c34)cc2c1]

N-(trans-4-Morpholinocyclohexyl)-5-(quinolin-6-yl)-7H-pyrrolo [2,3-d] pyrimidin-4-amine 1898.

Off-white solid (14.9 mg, 0.035 mmol, 40.5% yield). ¹H NMR (500 MHz, DMSO-d₆) δppm 1.01-1.10 (2 H, m), 1.23-1.36 (2 H, m), 1.73-1.80 (2 H, m), 2.02-2.15 (3 H, m), 2.39-2.45 (4 H, m), 3.49-3.56 (4 H, m), 3.93-4.04 (1 H, m), 5.23 (1 H, d, J=7.68 Hz), 7.41 (1 H, d, J=1.92 Hz), 7.58 (1 H, dd, J=8.23, 4.39 Hz), 7.91 (1 H, dd, J=8.64, 2.06 Hz), 8.01 (1 H, d,

J=1.92 Hz), 8.10 (1 H, d, J=8.78 Hz), 8.22 (1 H, s), 8.35-8.41 (1 H, m), 8.90 (1 H, dd, J=4.12, 1.65 Hz), 11.93 (1 H, s); ESIMS found for C25H_{281\160} m/z 429.2 (M+1).

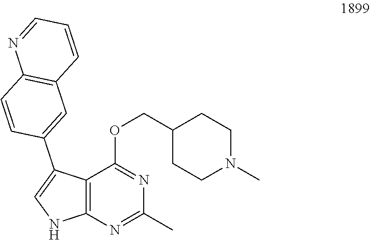 [SMILES: Cc1nc(OCC2CCN(C)CC2)c2c(-c3ccc4ncccc4c3)c[nH]c2n1]

6-(2-Methyl-4-((l-methylpiperidin-4-yl)methoxy)-7H-pyrrolo [2,3-d] pyrimidin-5-yl)quinoline 1899.

Beige solid (10 mg, 0.026 mmol, 21.9% yield). ¹H NMR (499 MHz, DMSO-d₆) δppm 1.28 (2 H, qd, J=12.08, 3.57 Hz), 1.65 (2 H, br d, J=12.35 Hz), 1.71-1.82 (3 H, m), 2.12 (3 H, s), 2.53 (3 H, s), 2.64-2.72 (2 H, m), 4.30 (2 H, d, J=6.59 Hz), 7.51 (1 H, dd, J=8.23, 4.12 Hz), 7.69 (1 H, s), 7.95 (1 H, d, J=8.78 Hz), 8.13 (1 H, dd, J=8.78, 1.92 Hz), 8.28 (1 H, d, J=1.92 Hz), 8.31 (1 H, d, J=7.68 Hz), 8.82 (1 H, dd, J=4.12, 1.65 Hz); ESIMS found for C₂₃H₂₅N₅O m/z 388.2 (M+1).

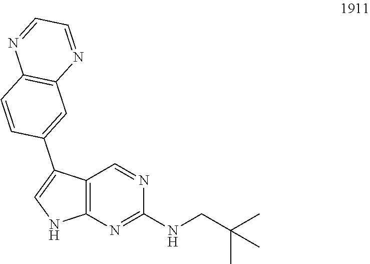 [SMILES: CC(C)(C)CNc1ncc2c(-c3ccc4nccnc4c3)c[nH]c2n1]

N-Neopentyl-5-(quinoxalin-6-yl)-7H-pyrrolo [2,3-d] pyrimidin-2-amine 1911.

White solid (24.97 mg, 0.075 mmol). ¹H NMR (400 MHz, DMSO-d₆) δppm 0.94 (9 H, s), 3.23 (2 H, br d, J=6.25 Hz), 6.79 (1 H, br s), 7.79 (1 H, s), 8.08 (1 H, d, J=8.50 Hz), 8.24-8.29 (2 H, m), 8.85 (1 H, d, J=1.38 Hz), 8.92 (1 H, d, J=1.38 Hz), 9.01 (1 H, s), 11.67 (1 H, br s); ESIMS found for C₁₉H₂₀N6 m/z 333.2 (M+1).

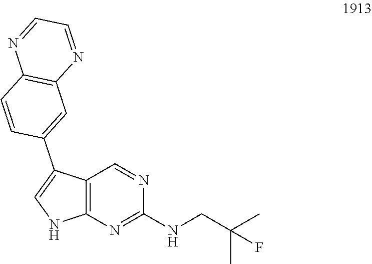 [SMILES: CC(C)(F)CNc1ncc2c(-c3ccc4nccnc4c3)c[nH]c2n1]

N-(2-Fluoro-2-methylpropyl)-5-(quinoxalin-6-yl)-7H-pyrrolo[2,3-d] pyrimidin-2-amine 1913.

White solid (4.1 mg, 0.012 mmol). ¹H NMR (400 MHz, DMSO-d₆) δppm 1.36 (6 H, d, J=21.40 Hz), 3.60 (2 H, dd, J=19.07, 6.48 Hz), 6.96 (1 H, br t, J=5.81 Hz), 7.83 (1 H, d, J=1.96 Hz), 8.09 (1 H, d, J=8.68 Hz), 8.25-8.31 (2 H, m), 8.86 (1 H, d, J=1.83 Hz), 8.92 (1 H, d, J=1.83 Hz), 9.04 (1 H, s), 11.74 (1 H, br s); ESIMS found for C₁₈H₁₇FN₆ m/z 337.0 (M+1).

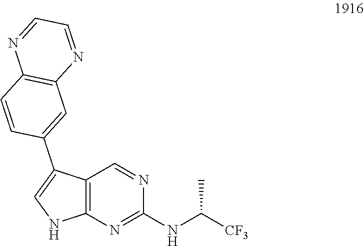 [SMILES: C[C@@H](Nc1ncc2c(-c3ccc4nccnc4c3)c[nH]c2n1)C(F)(F)F]

(R)-5-(Quinoxalin-6-yl)-N-(1,1,1-trifluoropropan-2-yl)-7H-pyrrolo [2,3-d] pyrimidin-2-amine 1916.

White solid (15.55 mg, 0.043 mmol). ¹H NMR (400 MHz, DMSO-d₆) δppm 1.37 (3 H, d, J=7.09 Hz), 4.88-5.05 (1 H, m), 7.47 (1 H, br d, J=8.93 Hz), 7.91 (1 H, d, J=1.71 Hz), 8.10 (1 H, d, J=8.68 Hz), 8.26-8.31 (1 H, m), 8.32 (1 H, d, J=1.83 Hz), 8.87 (1 H, d, J=1.83 Hz), 8.93 (1 H, d, J=1.83 Hz), 9.11 (1 H, s), 11.89 (1 H, br s); ESIMS found for C₁₇H₁₃F₃N₆ m/z 359.4 (M+1).

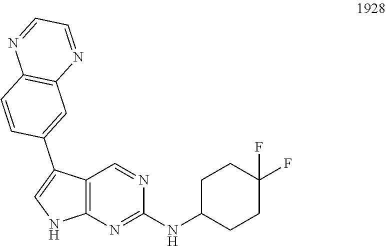 [SMILES: FC1(F)CCC(Nc2ncc3c(-c4ccc5nccnc5c4)c[nH]c3n2)CC1]

N-(4,4-Difluorocyclohexyl)-5-(quinoxalin-6-yl)-7H-pyrrolo [2,3-d]pyrimidin-2-amine 1928.

Yellow solid (5 mg, 0.013 mmol, 35.1% yield). ¹H NMR (499 MHz, DMSO-d₆) δppm 1.58-1.71 (2 H, m), 1.85-2.02 (4 H, m), 2.04-2.17 (2 H, m), 3.92-4.03 (1 H, m), 6.95 (1 H, br d, J=7.12 Hz), 7.82 (1 H, s), 8.08 (1 H, d, J=8.76 Hz), 8.25-8.30 (2 H, m), 8.86 (1 H, d, J=1.92 Hz), 8.92 (1 H, d, J=1.64 Hz), 9.04 (1 H, s), 11.76 (1 H, br s); ESIMS found for C₂₀F1₁₈F₂N₆ m/z 381.2 (M+1).

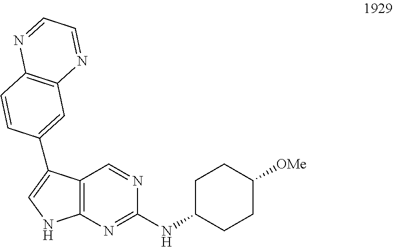 [SMILES: CO[C@H]1CC[C@@H](Nc2ncc3c(-c4ccc5nccnc5c4)c[nH]c3n2)CC1]

N-(cis-4-Methoxycyclohexyl)-5-(quinoxalin-6-yl)-7H-pyrrolo [2,3-d] pyrimidin-2-amine 1929.

White solid (8.15 mg, 0.022 mmol). ¹1-1 NMR (400 MHz, DMSO-d₆) δppm 1.44-1.54 (2 H, m), 1.54-1.65 (2 H, m), 1.65-1.73 (2 H, m), 1.86 (2 H, br d, J=10.38 Hz), 3.23 (3 H, s), 3.31-3.38 (1 H, m), 3.77-3.89 (1 H, m), 6.74 (1 H, br d, J=6.63 Hz), 7.79 (1 H, s), 8.08 (1 H, d, J=8.50 Hz), 8.23-8.29 (2 H, m), 8.85 (1 H, s), 8.91 (1 H, s), 9.01 (1 H, s), 11.72(1 H, br s); ESIMS found for C₂₁1⁻1₂₂N₆O m/z 375.2 (M+1).

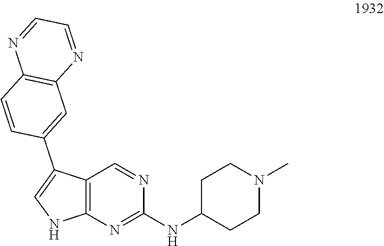 [SMILES: CN1CCC(Nc2ncc3c(-c4ccc5nccnc5c4)c[nH]c3n2)CC1]

N-(1-Methylpiperidin-4-yl)-5-(quinoxalin-6-yl)-7H-pyrrolo [2,3-d]pyrimidin-2-amine 1932.

White solid (9.70 mg, 0.027 mmol). ¹1-1 NMR (400 MHz, DMSO-d₆) δppm 1.53 (2 H, qd, J=11.62, 3.30 Hz), 1.88 (2 H, br d, J=10.51 Hz), 1.96 (2 H, br t, J=11.49 Hz), 2.17 (3 H, s), 2.76(2 H, br d, J=11.62 Hz), 3.66-3.76 (1 H, m), 6.73 (1 H, br d, J=7.95 Hz), 7.79 (1 H, s), 8.08 (1 H, d, J=8.80 Hz), 8.23-8.29 (2 H, m), 8.85 (1 H, d, J=1.83 Hz), 8.91 (1 H, d, J=1.83 Hz), 9.02 (1 H, s); ESIMS found for C₂₀H₂,N₇ m/z 360.6 (M+1).

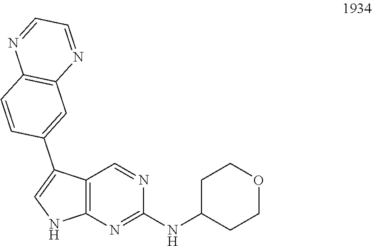 [SMILES: c1cnc2cc(-c3c[nH]c4nc(NC5CCOCC5)ncc34)ccc2n1]

5-(Quinoxalin-6-yl)-N-(tetrahydro -2H-pyran-4-yl)-7H-pyrrolo [2,3-d] pyrimidin-2-amine 1934.

White solid (24.93 mg, 0.072 mmol). ¹H NMR (400 MHz, DMSO-d₆) δppm 1.45-1.63 (2 H, m), 1.89 (2 H, br dd, J=12.47, 1.96 Hz), 3.36-3.46 (2 H, m), 3.90 (2 H, br d, J=11.25 Hz), 3.94-4.05 (1 H, m), 6.88 (1 H, br d, J=7.58 Hz), 7.81 (1 H, d, J=2.20 Hz), 8.08 (1 H, d, J=8.68 Hz), 8.24-8.30 (2 H, m), 8.85 (1 H, d, J=1.83 Hz), 8.92 (1 H, d, J=1.71 Hz), 9.04 (1 H, s), 11.75 (1 H, br s); ESIMS found for C₁₉H₁₈N60 m/z 347.1 (M+1).

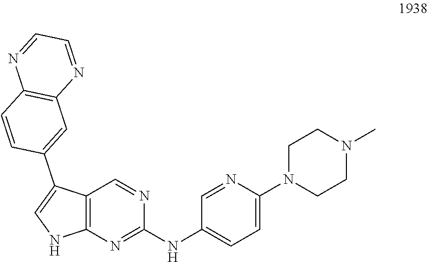 [SMILES: CN1CCN(c2ccc(Nc3ncc4c(-c5ccc6nccnc6c5)c[nH]c4n3)cn2)CC1]

N-(6-(4-Methylpipe razin-1-yl)pyridin-3-yl)-5-(quinoxalin-6-yl)-7H-pyrrolo [2,3-d]pyrimidin-2-amine 1938.

Brown solid (5 mg, 0.011 mmol, 24.2% yield). ¹H NMR (499 MHz, DMSO-d₆) δppm 2.22 (3 H, s), 2.39-2.46 (4 H, m), 3.37-3.44 (4 H, m), 6.83 (1 H, d, J=9.31 Hz), 7.94-7.97 (2 H, m), 8.11 (1 H, d, J=8.76 Hz), 8.31 (1 H, dd, J=8.76, 2.19 Hz), 8.35 (1 H, d, J=1.92 Hz), 8.58 (1 H, d, J=2.74 Hz), 8.87 (1 H, d, J=1.92 Hz), 8.94 (1 H, d, J=1.92 Hz), 9.18 (1 H, s), 9.21 (1 H, s), 11.95 (1 H, br s); ESIMS found for C24H₂₃N9 M/Z 438.1 (M+1).

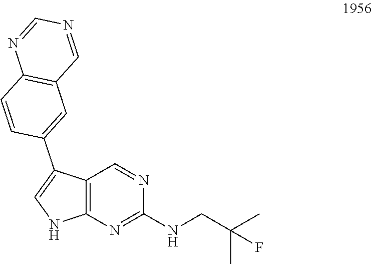 [SMILES: CC(C)(F)CNc1ncc2c(-c3ccc4ncncc4c3)c[nH]c2n1]

N-(2-Fluoro-2-methylpropyl)-5-(quinazolin-6-yl)-7H-pyrrolo [2,3-d] pyrimidin-2-amine 1956.

White solid (5.3 mg, 0.016 mmol). ¹H NMR (400 MHz, DMSO-d₆) δppm 1.36 (6 H, d, J=21.40 Hz), 3.61 (3 H, br dd, J=19.01, 6.50 Hz), 6.85-6.96 (1 H, m), 7.79 (1 H, s), 7.99 (1 H, d, J=8.63 Hz), 8.40-8.47 (2 H, m), 9.19 (1 H, s), 9.21 (1 H, s), 9.65 (1 H, s); ESIMS found for C₁₈H₁₇FN₆ m/z 337.1 (M+1).

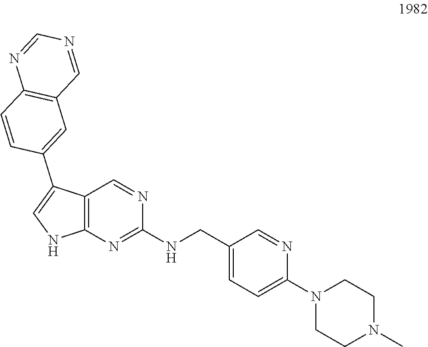 [SMILES: CN1CCN(c2ccc(CNc3ncc4c(-c5ccc6ncncc6c5)c[nH]c4n3)cn2)CC1]

N-((6-(4-Methylpiperazin-1-yl)pyridin-3-yl)methyl)-5-(quinazolin-6-yl)-7H-pyrrolo[2,3-d]pyrimidin-2-amine 1982.

Off-white solid (4.2 mg, 0.009 mmol, 51.2% yield). ¹H NMR (499 MHz, DMSO-d₆) δppm 2.19 (3 H, s), 2.33-2.40 (4 H, m), 3.39-3.44 (4 H, m), 4.41 (2 H, d, J=6.30 Hz), 6.77 (1 H, d, J=8.76 Hz), 7.34 (1 H, br t, J=5.48 Hz), 7.56 (1 H, dd, J=8.76, 2.46 Hz), 7.78 (1 H, s), 7.99 (1 H, d, J=8.76 Hz), 8.14 (1 H, d, J=2.19 Hz), 8.42 (1 H, dd, J=8.76, 1.92 Hz), 8.45 (1 H, d, J=1.92 Hz), 9.20 (1 H, s), 9.22 (1 H, s), 9.65 (1 H, s), 11.69 (1 H, br s); ESIMS found for C25H₂₅N9 MiZ 452.1 (M+1).

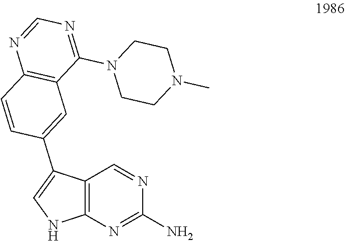 [SMILES: CN1CCN(c2ncnc3ccc(-c4c[nH]c5nc(N)ncc45)cc23)CC1]

5-(4-(4-Methylpiperazin-1-yl)quinazolin-6-yl)-7H-pyrrolo [2,3-d]pyrimidin-2-amine 1986.

Brown solid (30.0 mg, 0.083 mmol, 17.7% yield). ¹H NMR (499 MHz, DMSO-d₆) δppm 2.28 (3 H, s), 2.57 (4 H, t, J=4.65 Hz), 3.71-3.79 (4 H, m), 6.30 (2 H, s), 7.67 (1 H, s), 7.83 (1 H, d, J=8.76 Hz), 8.07 (1 H, d, J=1.92 Hz), 8.19 (1 H, dd, J=8.76, 1.92 Hz), 8.61 (1 H, s), 8.86 (1 H, s), 11.54 (1 H, s); ESIMS found for C₁₉H₂₀N₈ m/z 361.1 (M+1).

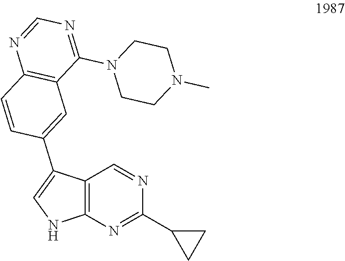 [SMILES: CN1CCN(c2ncnc3ccc(-c4c[nH]c5nc(C6CC6)ncc45)cc23)CC1]

6-(2—Cyclopropyl-7H-pyrrolo[2,3-d]pyrimidin-5-yl)-4-(4-methylpiperazin-1-yl)quinazoline 1987.

White solid (26.3 mg, 0.068 mmol, 30.7% yield). ¹H NMR (499 MHz, DMSO-d₆) δppm 1.01-1.07 (4 H, m), 2.23-2.26 (1 H, m), 2.27 (3 H, s), 2.52-2.58(4 H, m), 3.76-3.83 (4 H, m), 7.86 (1 H, d, J=8.49 Hz), 8.04 (1 H, s), 8.15 (1 H, d, J=1.92 Hz), 8.23 (1 H, dd, J=8.49, 1.92 Hz), 8.61 (1 H, s), 9.20 (1 H, s), 12.25 (1 H, br s); ESIMS found for C22H₂₃N7 M/Z 386.1 (M+1).

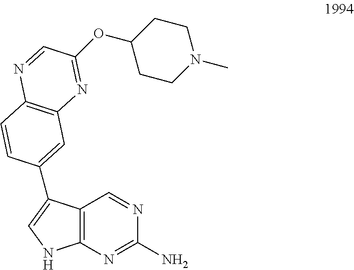 [SMILES: CN1CCC(Oc2cnc3ccc(-c4c[nH]c5nc(N)ncc45)cc3n2)CC1]

5-(3-((l-Methylpiperidin-4-yl)oxy)quinoxalin-6-yl)-7H-pyrrolo [2,3-d] pyrimidin-2-amine 1994.

Brown solid (8 mg, 0.021 mmol, 4.5% yield). ¹H NMR (499 MHz, DMSO-d₆) δppm 1.73-1.87 (2 H, m), 2.03-2.13 (2 H, m), 2.19-2.29 (2 H, m), 2.21 (3 H, s), 2.61-2.73 (2 H, m), 5.26 (1 H, tt, J=8.35, 3.97 Hz), 6.27 (2 H, s), 7.77 (1 H, s), 7.95-8.03 (3 H, m), 8.45 (1 H, s), 8.96 (1 H, s), 11.58 (1 H, br s); ESIMS found for C₂₀H_{2I}N₇O m/z 376.1 (M+1).

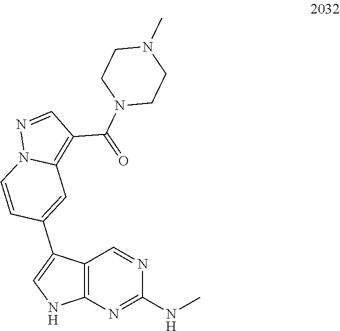 [SMILES: CNc1ncc2c(-c3ccn4ncc(C(=O)N5CCN(C)CC5)c4c3)c[nH]c2n1]

(5-(2-(Methylamino)-7H-pyrrolo [2,3-d] pyrimidin-5-yl)pyrazolo [1,5-a]pyridin-3-yl)(4-methylpiperazin-1-yl)methanone 2032.

Off-white solid (14 mg, 0.036 mmol, 29.1% yield). ¹H NMR (499 MHz, DMSO-d₆) δppm 2.21 (3 H, s), 2.37 (4 H, t, J=5.07 Hz), 2.84 (3 H, d, J=4.93 Hz), 3.63-3.73 (4 H, m), 6.85 (1 H, br d, J=3.83 Hz), 7.43 (1 H, dd, J=7.39, 1.92 Hz), 7.79 (1 H, s), 8.07 (1 H, d, J=1.37 Hz), 8.22 (1 H, s), 8.70-8.77 (1 H, m), 8.89 (1 H, s), 11.79 (1 H, br s); ESIMS found for C20H₂₂N80 m/z 391.1 (M+1).

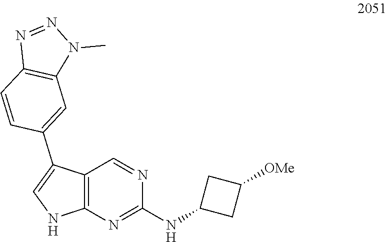 [SMILES: CO[C@H]1C[C@@H](Nc2ncc3c(-c4ccc5nnn(C)c5c4)c[nH]c3n2)C1]

N-(cis-3-Methoxycyclobutyl)-5-(1-methyl-1H-benzo[d] [1,2,3]triazol-6-yl)-7H-pyrrolo [2,3-d]pyrimidin-2-amine 2051.

White solid (24.46 mg, 0.070 mmol). ¹H NMR (400 MHz, DMSO-d₆) δppm 1.90 (2 H, qd, J=8.42, 2.75 Hz), 2.65-2.75 (2 H, m), 3.16 (3 H, s), 3.66 (1 H, dt, J=14.01, 7.13 Hz), 4.00 (1 H, dt, J=15.82, 7.85 Hz), 4.36 (3 H, s), 7.64-7.88 (1 H, m), 7.76 (1 H, dd, J=8.69, 1.31 Hz), 7.84 (1 H, br s), 8.04 (2 H, d, J=9.38 Hz), 9.13 (1 H, s), 12.14 (1 H, br s); ESIMS found for C₁₈H₁₉N70 m/z 350.1 (M+1).

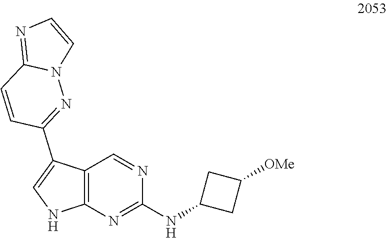 [SMILES: CO[C@H]1C[C@@H](Nc2ncc3c(-c4ccc5nccn5n4)c[nH]c3n2)C1]

5-(Imidazo[1,2-b]pyridazin-6-yl)-N-(cis-3-methoxycyclobutyl)-7H-pyrrolo [2,3-d]pyrimidin-2-amine 2053.

White solid (16.14 mg, 0.048 mmol). ¹H NMR (400 MHz, DMSO-d₆) δppm 1.82 (2 H, qd, J=8.48, 2.69 Hz), 2.57-2.69 (2 H, m), 3.14 (3 H, s), 3.55-3.65 (1 H, m), 3.93-4.07 (1 H, m), 6.82 (1 H, br d, J=6.60 Hz), 7.61 (1 H, s), 7.67 (1 H, d, J=9.66 Hz), 7.94 (1 H, d, J=9.54 Hz), 7.99 (1 H, s), 8.20 (1 H, s), 9.16 (1 H, s); ESIMS found for C₁₇H₁₇N₇O m/z 336.2 (M+1).

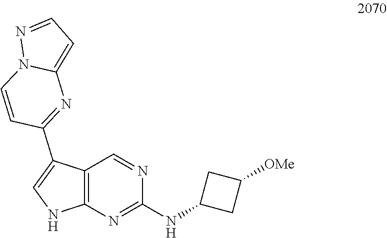 [SMILES: CO[C@H]1C[C@@H](Nc2ncc3c(-c4ccn5nccc5n4)c[nH]c3n2)C1]

N-(cis-3-Methoxycyclobutyl)-5-(pyrazolo[1,5-a]pyrimidin-5-yl)-7H-pyrrolo [2,3-d]pyrimidin-2-amine 2070.

White solid (36.21 mg, 0.108 mmol). ¹H NMR (400 MHz, DMSO-d₆) δppm 1.77-1.94 (2 H, m), 2.57-2.70 (2 H, m), 3.14 (3 H, s), 3.61 (1 H, quin, J=7.06 Hz), 3.91-4.07 (1 H, m), 6.60 (1 H, d, J=1.34 Hz), 7.22 (1 H, br dd, J=2.57, 1.83 Hz), 7.47 (1 H, d, J=7.34 Hz), 8.10 (1 H, d, J=1.96 Hz), 8.12 (1 H, s), 8.95 (1 H, d, J=7.34 Hz), 9.32 (1 H, s), 11.86 (1 H, br s); ESIMS found for C₁₇H₁₇N70 m/z 336.2 (M+1).

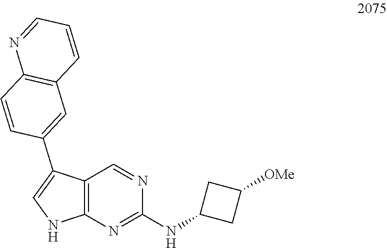 [SMILES: CO[C@H]1C[C@@H](Nc2ncc3c(-c4ccc5ncccc5c4)c[nH]c3n2)C1]

N-(cis-3-Methoxycyclobutyl)-5-(quinolin-6-yl)-7H-pyrrolo [2,3-d]pyrimidin-2-amine 2075.

White solid (12.82 mg, 0.037 mmol). ¹H NMR (400 MHz, DMSO-d₆) δppm 1.79-1.93 (2 H, m), 2.58-2.70 (2 H, m), 3.15 (3 H, s), 3.56-3.67 (1 H, m), 3.94-4.10 (1 H, m), 7.10 (1 H, br d, J=7.70 Hz), 7.51 (1 H, dd, J=8.31, 4.16 Hz), 7.68 (1 H, s), 7.99 (1 H, d, J=8.93 Hz), 8.14 (1 H, dd, J=8.86, 2.02 Hz), 8.28 (1 H, d, J=1.83 Hz), 8.44 (1 H, d, J=7.46 Hz), 8.81 (1 H, dd, J=4.22, 1.65 Hz), 9.12 (1 H, s), 11.49 (1 H, br s); ESIMS found for C₂oH₁₉N₅O m/z 346.5 (M+1).

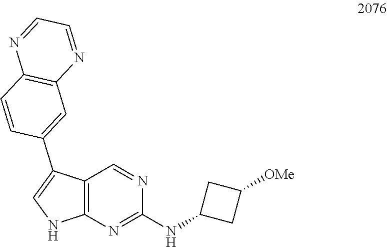 [SMILES: CO[C@H]1C[C@@H](Nc2ncc3c(-c4ccc5nccnc5c4)c[nH]c3n2)C1]

N-(cis-3-Methoxycyclobutyl)-5-(quinoxalin-6-yl)-7H-pyrrolo [2,3-d] pyrimidin-2-amine 2076.

White solid (19.43 mg, 0.056 mmol). ¹H NMR (400 MHz, DMSO-d₆) δppm 1.79-1.90 (2 H, m), 2.60-2.70 (2 H, m), 3.15 (3 H, s), 3.56-3.67 (1 H, m), 4.01 (1 H, dq, J=15.74, 7.71 Hz), 7.16 (1 H, br d, J=7.34 Hz), 7.81 (1 H, d, J=2.20 Hz), 8.08 (1 H, d, J=8.68 Hz), 8.24-8.29 (2 H, m), 8.85 (1 H, d, J=1.83 Hz), 8.92 (1 H, d, J=1.83 Hz), 9.02 (1 H, s), 11.73 (1 H, br s); ESIMS found for C₁₉H_{181\160} m/z 347.1 (M+1).

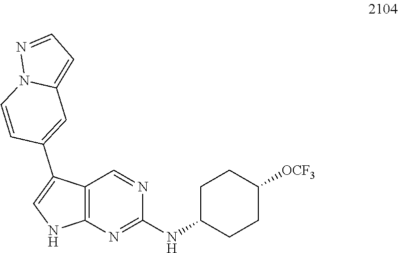

5-(Pyrazolo[1,5-a]pyridin-5-yl)-N-(cis-4-(trifluoromethoxy)cyclohexyl)-7H-pyrrolo [2,3-d]pyrimidin-2-amine 2104.

Grey solid (10 mg, 0.024 mmol, 29.7% yield). ¹H NMR (499 MHz, DMSO-d₆) δppm 1.60-1.70 (2 H, m), 1.70-1.78 (2 H, m), 1.82 (2 H, td, J=8.49, 3.83 Hz), 1.93-2.01 (2 H, m), 3.78-3.93 (1 H, m), 4.56-4.65 (1 H, m), 6.56 (1 H, d, J=2.19 Hz), 6.86 (1 H, br d, J=6.57 Hz), 7.24 (1 H, dd, J=7.39, 1.92 Hz), 7.67 (1 H, d, J=2.19 Hz), 7.94 (1 H, d, J=2.19 Hz), 7.97 (1 H, d, J=1.64 Hz), 8.61 (1 H, d, J=7.12 Hz), 9.04 (1 H, s), 11.60 (1 H, br s); ESIMS found for C₂₀H₁₉F3N60 m/z 417.2 (M+1).

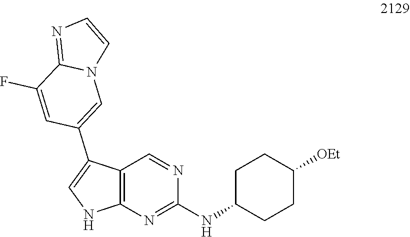

N-(cis-4-Ethoxycyclohexyl)-5-(8-fluoroimidazo [1,2-a]pyridin-6-yl)-7H-pyrrolo [2,3-d]pyrimidin-2-amine 2129.

White solid (6.51 mg, 0.017 mmol). ¹H NMR (400 MHz, DMSO-d₆) δppm 1.12 (3 H, t, J=6.94 Hz), 1.43-1.55 (2 H, m), 1.56-1.75 (4 H, m), 1.77-1.88 (2 H, m), 3.42 (2 H, q, J=7.00 Hz), 3.45-3.48 (1 H, m), 3.73-3.85 (1 H, m), 6.69 (1 H, br d, J=7.25 Hz), 7.53-7.62 (3 H, m), 8.09 (1 H, d, J=2.25 Hz), 8.82 (1 H, d, J=0.88 Hz), 9.02 (1 H, s), 11.55 (1 H, br s); ESIMS found for C2J⁻123FN60 m/z 395.2 (M+1).

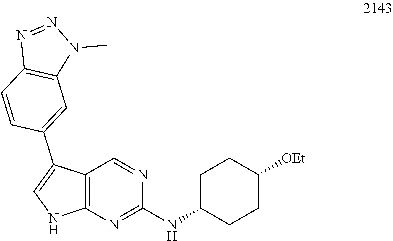

N-(cis-4-Ethoxycyclohexyl)-5-(1-methyl-1H-benzo[d][1,2,3]triazol-6-yl)-7H-pyrrolo [2,3-d]pyrimidin-2-amine 2143.

White solid (65.37 mg, 0.167 mmol). ¹H NMR (400 MHz, DMSO-d₆) δppm 1.13 (3 H, t, J=7.00 Hz), 1.42-1.56 (2 H, m), 1.57-1.75 (4 H, m), 1.79-1.90(2 H, m), 3.42(2 H, q, J=7.05 Hz), 3.46 (1 H, br s), 3.76-3.88 (1 H, m), 4.35 (3 H, s), 6.65 (1 H, br d, J=7.63 Hz), 7.60 (1 H, d, J=2.25 Hz), 7.76 (1 H, dd, J=8.76, 1.38 Hz), 7.99 (1 H, d, J=8.76 Hz), 8.02 (1 H, s), 9.09 (1 H, s), 11.56 (1 H, br s); ESIMS found for C₂J-1₂₅N₇O m/z 392.2 (M+1).

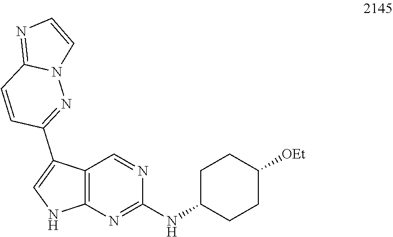

N-(cis-4-Ethoxycyclohexyl)-5-(imidazo[1,2-b]pyridazin-6-yl)-7H-pyrrolo [2,3-d]pyrimidin-2-amine 2145.

White solid (27.02 mg, 0.072 mmol). ¹H NMR (400 MHz, DMSO-d₆) δppm 1.13 (3 H, t, J=7.00 Hz), 1.45-1.55 (2 H, m), 1.60-1.73 (4 H, m), 1.78-1.88 (2 H, m), 3.42 (2 H, q, J=7.00 Hz), 3.46 (1 H, br s), 3.79 (1 H, br dd, J=7.44, 3.94 Hz), 6.77 (1 H, br d, J=6.88 Hz), 7.68 (1 H, d, J=1.00 Hz), 7.73 (1 H, d, J=9.51 Hz), 8.03 (1 H, br d, J=13.63 Hz), 8.02 (1 H, d, J=1.00 Hz), 8.29 (1 H, s), 9.26 (1 H, s), 11.78 (1 H, br s); ESIMS found for C₂₀I-1₂₃N₇O m/z 378.1 (M+1).

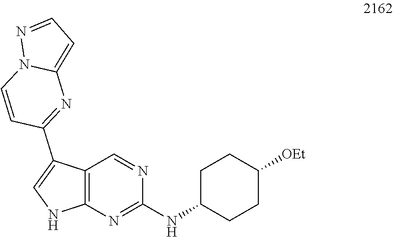

N-(cis-4-Ethoxycyclohexyl)-5-(pyrazolo [1,5-a]pyrimidin-5-yl)-7H-pyrrolo [2,3-d]pyrimidin-2-amine 2162.

White solid (72.11 mg, 0.191 mmol). ¹H NMR (400 MHz, DMSO-d₆) δppm 1.12 (3 H, t, J=7.00 Hz), 1.44-1.56 (2 H, m), 1.58-1.74 (5 H, m), 1.78-1.88 (2 H, m), 3.42 (2 H, q, J=6.92 Hz), 3.45 (1 H, br s), 3.72-3.87 (1 H, m), 6.59 (1 H, d, J=2.13 Hz), 6.78 (1 H, br d, J=6.75 Hz), 7.46 (1 H, d, J=7.50 Hz), 8.10 (2 H, d, J=2.25 Hz), 8.95 (1 H, d, J=7.50 Hz), 9.32 (1 H, s), 11.84 (1 H, br s); ESIMS found for C₂₀H₂₃N₇O m/z 378.1 (M+1).

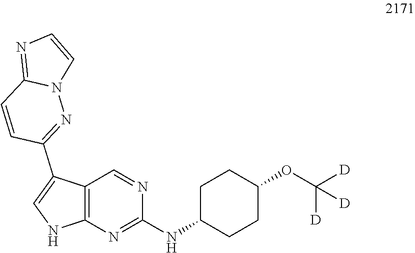 [SMILES: [2H]C([2H])([2H])O[C@H]1CC[C@@H](Nc2ncc3c(-c4ccc5nccn5n4)c[nH]c3n2)CC1]

5-(Imidazo [1,2-b] pyridazin-6-yl)-N-(cis-4-(methoxy-d₃)cyclohexyl)-7H-pyrrolo [2,3-d]pyrimidin-2-amine 2171.

White solid (41.91 mg, 0.115 mmol). ¹H NMR (400 MHz, DMSO-d₆) δppm 1.42-1.54 (2 H, m), 1.54-1.65 (2 H, m), 1.65-1.73 (2 H, m), 1.80-1.92 (2 H, m), 3.33-3.39 (1 H, m), 3.74-3.86 (1 H, m), 6.77 (1 H, br d, J=6.88 Hz), 7.68 (1 H, d, J=1.00 Hz), 7.73 (1 H, d, J=9.63 Hz), 8.01-8.06 (2 H, m), 8.29 (1 H, s), 9.26 (1 H, s), 11.78 (1 H, br s); ESIMS found for C₁₉H_{18[2}H_{3]1\170} m/z 367.2 (M+1).

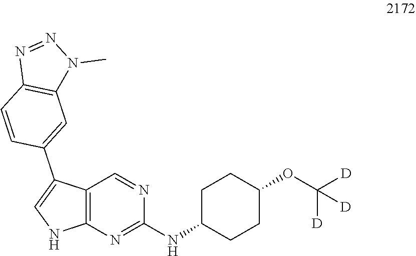 [SMILES: [2H]C([2H])([2H])O[C@H]1CC[C@@H](Nc2ncc3c(-c4ccc5nnn(C)c5c4)c[nH]c3n2)CC1]

N-(cis-4-(Methoxy-d₃)cyclohexyl)-5-(1-methyl-1H-benzo[d][1,2,3]triazol-6-yl)-7H-pyrrolo[2,3-d]pyrimidin-2-amine 2172.

White solid (75.08 mg, 0.198 mmol). ¹H NMR (400 MHz, DMSO-d₆) δppm 1.43-1.54 (2 H, m), 1.54-1.65 (2 H, m), 1.65-1.73 (2 H, m), 1.85 (2 H, br dd, J=8.69, 4.44 Hz), 3.35 (1 H, br s), 3.76-3.88 (1 H, m), 4.35 (3 H, s), 6.65 (1 H, br d, J=7.38 Hz), 7.60 (1 H, d, J=2.13 Hz), 7.76 (1 H, dd, J=8.76, 1.25 Hz), 7.99 (1 H, d, J=8.76 Hz), 8.02 (1 H, s), 9.09 (1 H, s), 11.56 (1 H, br s); ESIMS found for C₂₀I-1₂₀[2H₃]N₇O m/z 381.2 (M+1).

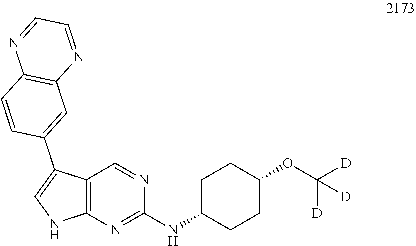 [SMILES: [2H]C([2H])([2H])O[C@H]1CC[C@@H](Nc2ncc3c(-c4ccc5nccnc5c4)c[nH]c3n2)CC1]

N-(cis-4-(Methoxy-d₃)cyclohexyl)-5-(quinoxalin-6-yl)-7H-pyrrolo [2,3-d] pyrimidin-2-amine 2173.

White solid (52.4 mg, 0.139 mmol). ¹H NMR (400 MHz, DMSO-d₆) δppm 1.44-1.54 (2 H, m), 1.55-1.65 (2 H, m), 1.65-1.73 (2 H, m), 1.81-1.92 (2 H, m), 3.76-3.90 (1 H, m), 6.71 (1 H, br d, J=7.38 Hz), 7.79 (1 H, s), 8.07 (1 H, d, J=9.38 Hz), 8.23-8.28 (2 H, m), 8.85 (1 H, d, J=1.88 Hz), 8.91 (1 H, d, J=1.75 Hz), 9.01 (1 H, s), 11.58 (1 H, s); ESIMS found for C₂₁H₁₉[2H₃]N₆O m/z 378.2 (M+1).

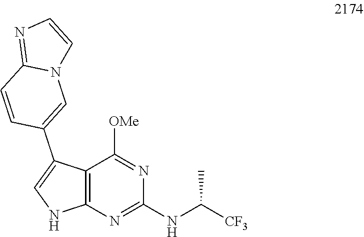 [SMILES: COc1nc(N[C@H](C)C(F)(F)F)nc2[nH]cc(-c3ccc4nccn4c3)c12]

(R)-5-(Imidazo [1,2-a] pyridin-6-yl)-4-methoxy-N-(1,1,1-trifluoropropan-2-yl)-7H-pyrrolo [2,3-d] pyrimidin-2-amine 2174.

White solid (9.28 mg, 0.025 mmol). ¹H NMR (400 MHz, DMSO-d₆) δppm 1.35 (3 H, d, J=7.00 Hz), 3.98 (3 H, s), 4.86-5.00 (1 H, m), 7.23-7.29 (2 H, m), 7.48-7.53 (2 H, m), 7.54 (1 H, s), 7.96 (1 H, s), 8.78 (1 H, s), 11.59 (1 H, br s); ESIMS found for C₁₇H₁₅F₃N₆O m/z 377.0 (M+1).

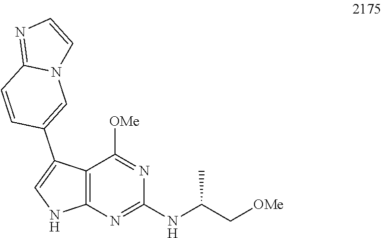 [SMILES: COC[C@@H](C)Nc1nc(OC)c2c(-c3ccc4nccn4c3)c[nH]c2n1]

(R)-5-(Imidazo [1,2-a] pyridin-6-yl)-4-methoxy-N-(1-methoxypropan-2-yl)-7H-pyrrolo [2,3-d]pyrimidin-2-amine 2175.

White solid (82.48 mg, 0.234 mmol). ¹H NMR (400 MHz, DMSO-d₆) δppm 1.16 (3 H, d, J=6.63 Hz), 3.23-3.27 (1 H, m), 3.28 (3 H, s), 3.45 (1 H, br dd, J=9.19, 5.57 Hz), 3.96 (3 H, s), 4.11-4.25 (1 H, m), 6.39 (1 H, d, J=8.25 Hz), 7.19 (1 H, d, J=2.13 Hz), 7.53 (3 H, d, J=9.76 Hz), 7.96 (1 H, s), 8.78 (1 H, s), 11.45 (1 H, br s); ESIMS found for C₁₈H₂oN₆O₂ m/z 353.1 (M+1).

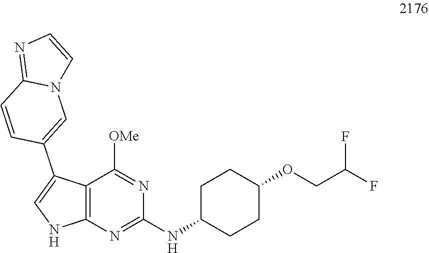 [SMILES: COc1nc(N[C@H]2CC[C@@H](OCC(F)F)CC2)nc2[nH]cc(-c3ccc4nccn4c3)c12]

N-(cis-4-(2,2-Difluoroethoxy)cyclohexyl)-5-(imidazo[1,2-a]pyridin-6-yl)-4-methoxy-7H-pyrrolo [2,3-d]pyrimidin-2-amine 2176.

Beige solid (24 mg, 0.054 mmol, 35.1% yield). ¹H NMR (499 MHz, DMSO-d₆) δppm 1.49-1.57 (2 H, m), 1.59-1.66 (2 H, m), 1.66-1.73 (2 H, m), 1.80-1.91 (2 H, m), 3.56-3.61 (1 H, m), 3.67 (2 H, td, J=15.19, 3.83 Hz), 3.75-3.82 (1 H, m), 3.95 (3 H, s), 6.13 (1 H, tt, J=55.12, 3.85 Hz), 6.52 (1 H, d, J=7.39 Hz), 7.17 (1 H, d, J=2.19 Hz), 7.48-7.52 (2 H, m), 7.54 (1 H, d, J=0.82 Hz), 7.96 (1 H, s), 8.77 (1 H, s), 11.43 (1 H, br s); ESIMS found for C₂₂H₂₄F₂N₆O₂ m/z 443.2 (M+1).

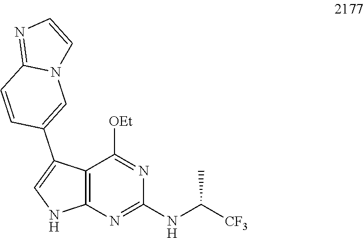 [SMILES: CCOc1nc(N[C@H](C)C(F)(F)F)nc2[nH]cc(-c3ccc4nccn4c3)c12]

(R)-4-Ethoxy-5-(imidazo [1,2-a]pyridin-6-yl)-N-(1,1,1-trifluoropropan-2-yl)-7H-pyrrolo [2,3-d]pyrimidin-2-amine 2177.

White solid (24.96 mg, 0.064 mmol). ¹H NMR (400 MHz, DMSO-d₆) δppm 1.35 (2 H, d, J=6.52 Hz), 1.37 (3 H, t, J=7.10 Hz), 4.46 (2 H, q, J=7.00 Hz), 4.91 (1 H, dq, J=15.13, 7.80 Hz), 7.21 (1 H, d, J=9.01 Hz), 7.27 (1 H, d, J=2.38 Hz), 7.52-7.55 (3 H, m), 7.91 (1 H, s), 8.83 (1 H, s), 11.56 (1 H, br s); ESIMS found for C₁₈H₁₇F₃N₆O m/z 391.1 (M+1).

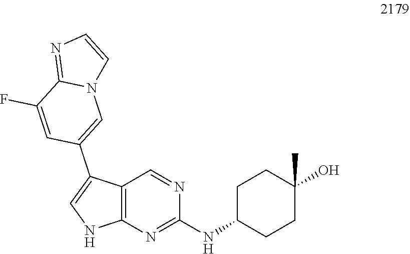 [SMILES: C[C@]1(O)CC[C@@H](Nc2ncc3c(-c4cc(F)c5nccn5c4)c[nH]c3n2)CC1]

cis-4-((5-(8-Fluoroimidazo [1,2-a]pyridin-6-yl)-7H-pyrrolo [2,3-d]pyrimidin-2-yl)amino)-1-methylcyclohexan-1-ol 2179.

Beige solid (110 mg, 0.289 mmol, 56.8% yield). ¹H NMR (499 MHz, DMSO-d₆) δppm 1.13 (3 H, s), 1.35 (2 H, td, J=12.87, 4.65 Hz), 1.58 (2 H, br d, J=12.05 Hz), 1.63-1.73 (4 H, m), 3.59-3.73 (1 H, m), 3.99 (1 H, s), 6.63 (1 H, br d, J=5.20 Hz), 7.57-7.61 (3 H, m), 8.04-8.12 (1 H, m), 8.82 (1 H, d, J=1.37 Hz), 9.02 (1 H, s), 11.54 (1 H, br s); ESIMS found for C20H₂IFN₆O m/z 381.2 (M+1).

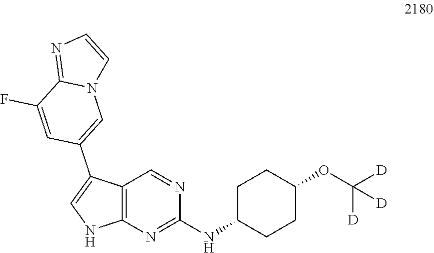 [SMILES: [2H]C([2H])([2H])O[C@H]1CC[C@@H](Nc2ncc3c(-c4cc(F)c5nccn5c4)c[nH]c3n2)CC1]

5-(8-Fluoroimidazo [1,2-a]pyridin-6-yl)-N-(cis-4-(methoxy-d₃)cyclohexyl)-7H-pyrrolo [2,3-d]pyrimidin-2-amine 2180.

White solid (13.3 mg, 0.035 mmol). ¹H NMR (400 MHz, DMSO-d₆) δppm 1.44-1.54 (2 H, m), 1.54-1.65 (2 H, m), 1.65-1.73 (2 H, m), 1.83 (2 H, br dd, J=8.74, 4.34 Hz), 3.32-3.38 (1 H, m), 3.75-3.87 (1 H, m), 6.85 (1 H, br d, J=2.69 Hz), 7.58-7.63 (2 H, m), 7.65 (1 H, d, J=2.32 Hz), 8.10 (1 H, dd, J=3.00, 0.92 Hz), 8.83 (1 H, d, J=1.22 Hz), 9.03 (1 H, s), 11.71 (1 H, br s); ESIMS found for C₂₀H₁₈[2H₃]FN₆O m/z 384.1 (M+1).

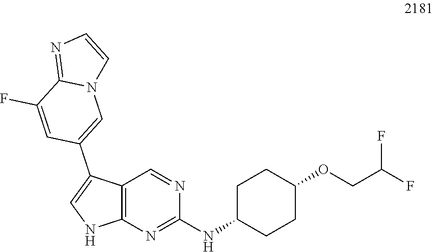 [SMILES: Fc1cc(-c2c[nH]c3nc(N[C@H]4CC[C@@H](OCC(F)F)CC4)ncc23)cn2ccnc12]

N-(cis-4-(2,2-Difluoroethoxy)cyclohexyl)-5-(8-fluoroimidazo [1,2-a]pyridin-6-yl)-7H-pyrrolo [2,3-d] pyrimidin-2-amine 2181.

White solid (33.4 mg, 0.078 mmol). ¹H NMR (400 MHz, DMSO-d₆) δppm 1.48-1.58 (2 H, m), 1.58-1.66 (2 H, m), 1.67-1.75 (2 H, m), 1.81-1.90 (2 H, m), 3.56-3.61 (1 H, m), 3.67 (2 H, td, J=15.20, 3.75 Hz), 3.75-3.85 (1 H, m), 6.12 (1 H, tt, J=55.12, 3.64 Hz), 6.72 (1 H, br d, J=7.25 Hz), 7.55-7.62 (3 H, m), 8.09 (1 H, d, J=2.50 Hz), 8.82 (1 H, s), 9.03 (1 H, s), 11.55 (1 H, br s); ESIMS found for C2,H_{2,}F3N60 m/z 431.2 (M+1).

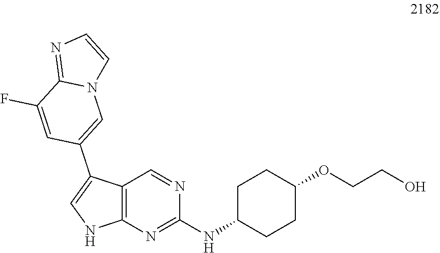 [SMILES: OCCO[C@H]1CC[C@@H](Nc2ncc3c(-c4cc(F)c5nccn5c4)c[nH]c3n2)CC1]

2-((cis-4-((5-(8-Fluoroimidazo [1,2-a] pyridin-6-yl)-7H-pyrrolo [2,3-d]pyrimidin-2-yl)amino)cyclohexyl)oxy)ethan-1-ol 2182.

White solid (27.46 mg, 0.067 mmol). ¹H NMR (400 MHz, DMSO-d₆) δppm 1.44-1.55 (2 H, m), 1.59-1.73 (4 H, m), 1.84 (2 H, br dd, J=13.20, 3.81 Hz), 3.40 (2 H, t, J=5.36 Hz), 3.45-3.48 (1 H, m), 3.51 (2 H, q, J=5.52 Hz), 3.72-3.84 (1 H, m), 4.51 (1 H, t, J=5.50 Hz), 6.67 (1 H, br d, J=6.75 Hz), 7.56-7.61 (3 H, m), 8.09 (1 H, d, J=2.25 Hz), 8.82 (1 H, s), 9.02 (1 H, s), 11.55 (1 H, br s); ESIMS found for C₂,H₂₃FN₆O₂ m/z 411.2 (M+1).

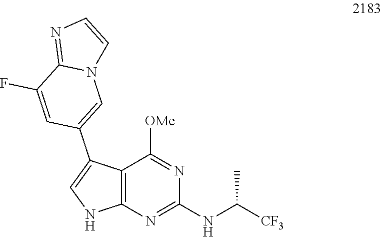 [SMILES: COc1nc(N[C@H](C)C(F)(F)F)nc2[nH]cc(-c3cc(F)c4nccn4c3)c12]

(R)-5-(8-Fluoroimidazo [1,2-a]pyridin-6-yl)-4-methoxy-N-(1,1,1-trifluoropropan-2-yl)-7H-pyrrolo [2,3-d]pyrimidin-2-amine 2183.

White solid (32.6 mg, 0.083 mmol). ¹H NMR (400 MHz, DMSO-d₆) δppm 1.35 (3 H, br d, J=7.00 Hz), 4.01 (3 H, s), 4.85-5.00 (1 H, m), 7.28 (1 H, br d, J=9.01 Hz), 7.35 (1 H, d, J=1.13 Hz), 7.46 (1 H, br d, J=12.88 Hz), 7.60 (1 H, s), 8.12 (1 H, d, J=2.88 Hz), 8.72 (1 H, s), 11.65 (1 H, br s); ESIMS found for C₁₇H₁₄F4N60 m/z 395.3 (M+1).

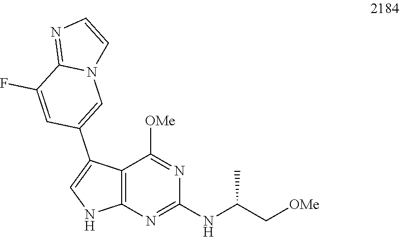 [SMILES: COC[C@@H](C)Nc1nc(OC)c2c(-c3cc(F)c4nccn4c3)c[nH]c2n1]

(R)-5-(8-Fluoroimidazo[1,2-a]pyridin-6-yl)-4-methoxy-N-(1-methoxypropan-2-yl)-7H-pyrrolo[2,3-d]pyrimidin-2-amine 2184.

White solid (13.66 mg, 0.037 mmol). ¹H NMR (400 MHz, DMSO-d₆) δppm 1.16 (3 H, d, J=6.63 Hz), 3.23-3.27 (1 H, m), 3.28 (3 H, s), 3.45 (1 H, br dd, J=9.13, 5.63 Hz), 3.98 (3 H, s), 4.17 (1 H, dquin, J=13.45, 6.52, 6.52, 6.52, 6.52 Hz), 6.43 (1 H, d, J=8.25 Hz), 7.28 (1 H, d, J=2.00 Hz), 7.45 (1 H, d, J=12.88 Hz), 7.59 (1 H, s), 8.11 (1 H, d, J=2.25 Hz), 8.72 (1 H, s), 11.51 (1 H, br s); ESIMS found for C₁₈H₁₉FN₆O₂ m/z 371.1 (M+1).

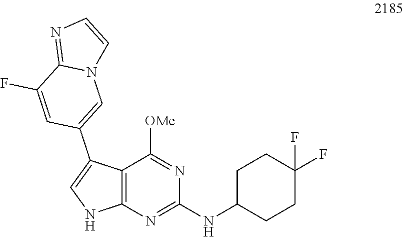 [SMILES: COc1nc(NC2CCC(F)(F)CC2)nc2[nH]cc(-c3cc(F)c4nccn4c3)c12]

N-(4,4-Difluorocyclohexyl)-5-(8-fluoroimidazo[1,2-a]pyridin-6-yl)-4-methoxy-7H-pyrrolo [2,3-d]pyrimidin-2-amine 2185.

White solid (0.96 mg, 0.002 mmol). ¹H NMR (400 MHz, DMSO-d₆) δppm 1.57-1.71 (2 H, m), 1.80-2.03 (4 H, m), 2.03-2.17 (2 H, m), 3.86-3.97 (1 H, m), 3.99 (3 H, s), 6.73 (1 H, d, J=7 .7 5 Hz), 7.28 (1 H, d, J=2.25 Hz), 7.42-7.50 (1 H, m), 7.59 (1 H, d, J=0.88 Hz), 8.07-8.14 (1 H, m), 8.72 (1 H, d, J=1.13 Hz), 11.52 (1 H, br s); ESIMS found for C₂oH₁₉F₃N₆O m/z 417.2 (M+1).

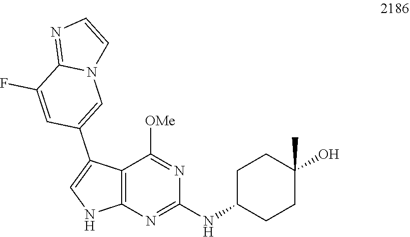 [SMILES: COc1nc(N[C@H]2CC[C@@](C)(O)CC2)nc2[nH]cc(-c3cc(F)c4nccn4c3)c12]

cis-4-((5-(8-Fluoroimidazo [1,2-a]pyridin-6-yl)-4-methoxy-7H-pyrrolo [2,3-d]pyrimidin-2-yl)amino)-1-methylcyclohexan-1-ol 2186.

Beige solid (24.5 mg, 0.060 mmol, 34.7% yield). ¹H NMR (499 MHz, DMSO-d₆) δppm 1.13 (3 H, s), 1.35 (2 H, td, J=12.73, 4.65 Hz), 1.58 (2 H, br d, J=12.59 Hz), 1.62-1.73 (4 H, m), 3.59-3.72 (1 H, m), 3.97 (3 H, s), 3.98 (1 H, s), 6.44 (1 H, d, J=7.67 Hz), 7.25 (1 H, d, J=2.19 Hz), 7.40-7.49 (1 H, m), 7.59 (1 H, d, J=1.10 Hz), 8.11 (1 H, d, J=2.19 Hz), 8.72 (1 H, d, J=1.10 Hz), 11.47 (1 H, br s); ESIMS found for C_{2I}F1₂₃FN₆O₂ m/z 411.1 (M+1).

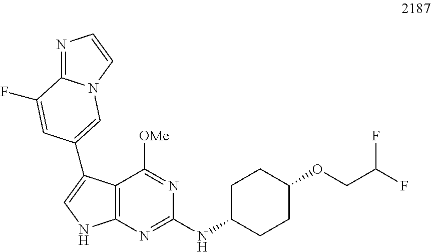 [SMILES: COc1nc(N[C@H]2CC[C@@H](OCC(F)F)CC2)nc2[nH]cc(-c3cc(F)c4nccn4c3)c12]

N-(cis-4-(2,2-Difluoroethoxy)cyclohexyl)-5-(8-fluoroimidazo[1,2-a]pyridin-6-yl)-4-methoxy-7H-pyrrolo [2,3-d] pyrimidin-2-amine 2187.

Beige solid (43 mg, 0.093 mmol, 31.3% yield). ¹H NMR (499 MHz, DMSO-d₆) δppm 1.49-1.57 (2 H, m), 1.59-1.66 (2 H, m), 1.66-1.73 (2 H, m), 1.82-1.89 (2 H, m), 3.56-3.61 (1 H, m), 3.67 (2 H, td, J=15.19, 3.83 Hz), 3.74-3.82 (1 H, m), 3.98 (3 H, s), 6.13 (1 H, tt, J=55.12, 3.85 Hz), 6.56 (1 H, d, J=7.67 Hz), 7.26 (1 H, d, J=2.19 Hz), 7.45 (1 H, dd, J=12.87, 1.10 Hz), 7.59 (1 H, d, J=1.10 Hz), 8.11 (1 H, dd, J=3.01, 1.09 Hz), 8.72 (1 H, d, J=1.09 Hz), 11.49 (1 H, br s); ESIMS found for C₂₂H₂₃F₃N₆O₂m/z 461.2 (M+1).

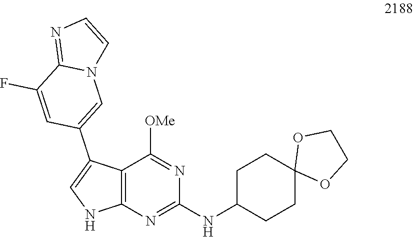 [SMILES: COc1nc(NC2CCC3(CC2)OCCO3)nc2[nH]cc(-c3cc(F)c4nccn4c3)c12]

5-(8-Fluoroimidazo[1,2-a]pyridin-6-yl)-4-methoxy-N-(1,4-dioxaspiro[4.5] decan-8-yl)-7H-pyrrolo [2,3-d]pyrimidin-2-amine 2188.

Beige solid (33 mg, 0.075 mmol, 47.0% yield). ¹H NMR (499 MHz, DMSO-d₆) δppm 1.49-1.65 (4 H, m), 1.70-1.78 (2 H, m), 1.87-1.95 (2 H, m), 3.76-3.83 (1 H, m), 3.83 -3.90 (4 H, m), 3.98 (3 H, s), 6.60 (1 H, d, J=7.67 Hz), 7.26 (1 H, d, J=2.19 Hz), 7.45 (1 H, dd, J=12.87, 1.10 Hz), 7.59 (1 H, d, J=1.10 Hz), 8.11 (1 H, dd, J=3.01, 1.10 Hz), 8.72 (1 H, d, J=1.37 Hz), 11.51 (1 H, br s); ESIMS found for C₂₂H₂₃FN₆O₃ m/z 439.2 (M+1).

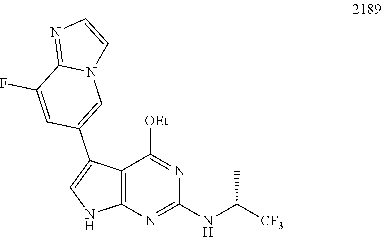 [SMILES: CCOc1nc(N[C@H](C)C(F)(F)F)nc2[nH]cc(-c3cc(F)c4nccn4c3)c12]

(R)-4-Ethoxy-5-(8-fluoroimidazo [1,2-a]pyridin-6-yl)-N-(1,1,1-trifluoropropan-2-yl)-7H-pyrrolo [2,3-d]pyrimidin-2-amine 2189.

White solid (35.97 mg, 0.088 mmol). ¹H NMR (400 MHz, DMSO-d₆) δppm 1.35 (3 H, br d, J=7.13 Hz), 1.38 (3 H, t, J=6.90 Hz), 4.47 (2 H, q, J=6.80 Hz), 4.91 (1 H, dq, J=14.95, 7.44 Hz), 7.23 (1 H, br d, J=9.01 Hz), 7.34 (1 H, s), 7.49 (1 H, br d, J=12.88 Hz), 7.60 (1 H, s), 8.05 (1 H, br s), 8.75 (1 H, s), 11.62 (1 H, br s); ESIMS found for C₁₈H₁₆F₄N₆O m/z 409.0 (M+1).

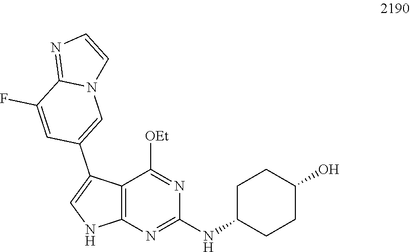 [SMILES: CCOc1nc(N[C@H]2CC[C@@H](O)CC2)nc2[nH]cc(-c3cc(F)c4nccn4c3)c12]

cis-4-((4-Ethoxy-5-(8-fluoroimidazo [1,2-a]pyridin-6-yl)-7H-pyrrolo [2,3-d]pyrimidin-2-yl)amino)cyclohexan-1-ol 2190.

White solid (31.41 mg, 0.077 mmol). ¹H NMR (400 MHz, DMSO-d₆) δppm 1.38 (3 H, t, J=7.07 Hz), 1.45-1.55 (2 H, m), 1.59-1.77 (6 H, m), 3.73 (2 H, br s), 4.32 (1 H, br s), 4.44 (2 H, q, J=7.00 Hz), 6.40 (1 H, d, J=7.63 Hz), 7.25 (1 H, d, J=2.25 Hz), 7.48 (1 H, dd, J=12.94, 0.94 Hz), 7.59 (1 H, d, J=0.88 Hz), 8.04 (1 H, d, J=2.13 Hz), 8.75 (1 H, d, J=1.00 Hz), 11.46 (1 H, br s); ESIMS found for C_{2I}F1₂₃FN₆O₂ m/z 411.2 (M+1).

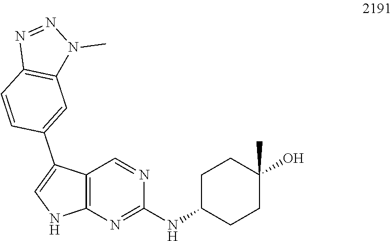 [SMILES: Cn1nnc2ccc(-c3c[nH]c4nc(N[C@H]5CC[C@@](C)(O)CC5)ncc34)cc21]

cis-1-Methyl-4-45-(1-methyl-1H-benzo[d][1,2,3]triazol-6-yl)-7H-pyrrolo [2,3-d] pyrimidin-2-yl)amino)cyclohexan-1-ol 2191.

Beige solid (44 mg, 0.117 mmol, 73.6% yield). ¹H NMR (499 MHz, DMSO-d₆) δppm 1.13 (3 H, s), 1.36 (2 H, td, J=12.87, 4.93 Hz), 1.59 (2 H, br d, J=12.05 Hz), 1.64-1.74 (4 H, m), 3.61-3.75 (1 H, m), 4.00 (1 H, s), 4.35 (3 H, s), 6.58 (1 H, br d, J=6.57 Hz), 7.59 (1 H, s), 7.76 (1 H, dd, J=8.76, 1.64 Hz), 7.99 (1 H, d, J=8.76 Hz), 8.02 (1 H, s), 9.08 (1 H, s), 11.55 (1 H, br s); ESIMS found for C₂₀I-1₂₃N₇O m/z 378.2 (M+1).

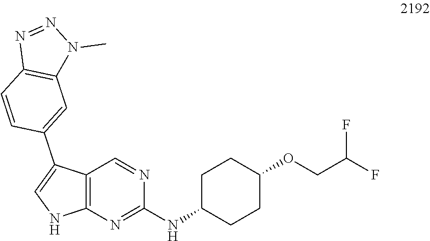 [SMILES: Cn1nnc2ccc(-c3c[nH]c4nc(N[C@H]5CC[C@@H](OCC(F)F)CC5)ncc34)cc21]

N-(cis-4-(2,2-Difluoroethoxy)cyclohexyl)-5-(1-methyl-1H-benzo [d] [1,2,3]triazol-6-yl)-7H-pyrrolo [2,3-d] pyrimidin-2-amine 2192.

White solid (31.2 mg, 0.073 mmol). ¹H NMR (400 MHz, DMSO-d₆) δppm 1.49-1.58 (2 H, m), 1.58-1.66 (2 H, m), 1.67-1.74 (2 H, m), 1.81-1.90 (2 H, m), 3.56-3.61 (1 H, m), 3.67 (2 H, td, J=15.25, 3.73 Hz), 3.76-3.86 (1 H, m), 4.35 (3 H, s), 6.13 (1 H, tt, J=55.12, 3.64 Hz), 6.69 (1 H, br d, J=6.97 Hz), 7.60 (1 H, d, J=1.47 Hz), 7.72-7.80 (1 H, m), 7.99 (1 H, d, J=8.80 Hz), 8.02 (1 H, s), 9.09 (1 H, s), 11.57 (1 H, br s); ESIMS found for C₂it1₂₃F₂N₇O m/z 428.2 (M+1).

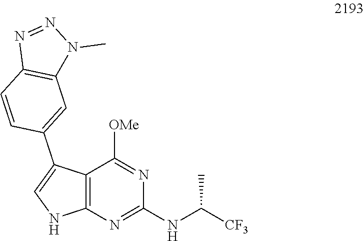 [SMILES: COc1nc(N[C@H](C)C(F)(F)F)nc2[nH]cc(-c3ccc4nnn(C)c4c3)c12]

(R)-4-Methoxy-5-(1-methyl-1H-benzo[d] [1,2,3]triazol-6-yl)-N-(1,1,1-trifluoropropan-2-yl)-7H-pyrrolo [2,3-d]pyrimidin-2-amine 2193.

White solid (72.25 mg, 0.185 mmol). ¹H NMR (400 MHz, DMSO-d₆) δppm 1.36 (3 H, d, J=7.00 Hz), 3.98 (3 H, s), 4.30 (3 H, s), 4.94 (1 H, dq, J=15.56, 7.61 Hz), 7.26 (1 H, d, J=9.01 Hz), 7.33 (1 H, d, J=2.38 Hz), 7.70 (1 H, dd, J=8.69, 1.44 Hz), 7.95 (1 H, d, J=8.76 Hz), 8.04 (1 H, s), 11.64 (1 H, br s); ESIMS found for C₁₇H₁₆F₃N₇O m/z 392.0 (M+1).

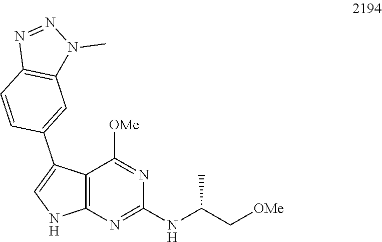 [SMILES: COC[C@@H](C)Nc1nc(OC)c2c(-c3ccc4nnn(C)c4c3)c[nH]c2n1]

(R)-4-Methoxy-N-(1-methoxypropan-2-yl)-5-(1-methyl-1H-benzo[d] [1,2,3]triazol-6-yl)-7H-pyrrolo [2,3-d]pyrimidin-2-amine 2194.

White solid (59.59 mg, 0.162 mmol). ¹H NMR (400 MHz, DMSO-d₆) δppm 1.17 (3 H, d, J=6.63 Hz), 3.24-3.29 (1 H, m), 3.28 (3 H, s), 3.46 (1 H, br dd, J=9.26, 5.50 Hz), 3.95 (3 H, s), 4.08-4.23 (1 H, m), 4.30 (3 H, s), 6.41 (1 H, d, J=8.25 Hz), 7.26 (1 H, d, J=2.13 Hz), 7.69 (1 H, dd, J=8.76, 1.50 Hz), 7.94 (1 H, d, J=8.76 Hz), 8.03 (1 H, s), 11.52 (1 H, br s); ESIMS found for C₁₈H₂iN702 m/z 368.1 (M+1).

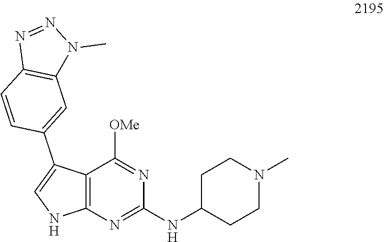 [SMILES: COc1nc(NC2CCN(C)CC2)nc2[nH]cc(-c3ccc4nnn(C)c4c3)c12]

4-Methoxy-5-(1-methyl-1H-benzo[d][1,2,3]triazol-6-yl)-N-(1-methylpiperidin-4-yl)-7H-pyrrolo [2,3-d]pyrimidin-2-amine 2195.

White solid (2.0 mg, 0.005 mmol). ¹H NMR (400 MHz, DMSO-d₆) δppm 1.46-1.62 (2 H, m), 1.84-1.91 (2 H, m), 1.95 (2 H, br t, J=11.37 Hz), 2.17 (3 H, s), 2.72-2.81 (2 H, m), 3.64-3.75 (1 H, m), 3.95 (3 H, s), 4.29 (3 H, s), 6.51 (1 H, d, J=7.95 Hz), 7.24 (1 H, d, J=2.08 Hz), 7.69 (1 H, dd, J=8.80, 1.47 Hz), 7.94 (1 H, d, J=9.05 Hz), 8.03 (1 H, s), 11.52 (1 H, br s); ESIMS found for C20H₂₄N80 m/z 393.2 (M+1).

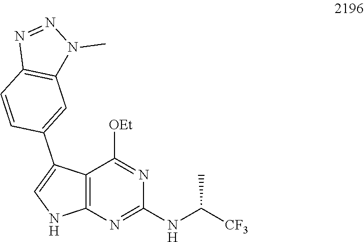 [SMILES: CCOc1nc(N[C@H](C)C(F)(F)F)nc2[nH]cc(-c3ccc4nnn(C)c4c3)c12]

(R)-4-Ethoxy-5-(1-methyl-1H-benzo[d] [1,2,3]triazol-6-yl)-N-(1,1,1-trifluoropropan-2-yl)-7H-pyrrolo [2,3-d]pyrimidin-2-amine 2196.

White solid (158.18 mg, 0.390 mmol). ¹H NMR (400 MHz, DMSO-d₆) δppm 1.35 (3 H, br d, J=7.09 Hz), 1.37 (3 H, br t, J=7.03 Hz), 4.30 (3 H, s), 4.48 (2 H, q, J=7.05 Hz), 4.92 (1 H, dq, J=15.07, 7.53 Hz), 7.23 (1 H, br d, J=9.05 Hz), 7.34 (1 H, d, J=2.45 Hz), 7.73 (1 H, dd, J=8.74, 1.28 Hz), 7.95 (1 H, d, J=8.68 Hz), 8.09 (1 H, s), 11.63 (1 H, br s); ESIMS found for C₁₈H₁₈F3N70 m/z 406.0 (M+1).

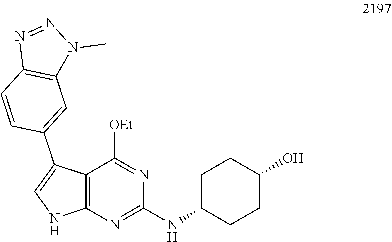 [SMILES: CCOc1nc(N[C@H]2CC[C@@H](O)CC2)nc2[nH]cc(-c3ccc4nnn(C)c4c3)c12]

cis-4-44-Ethoxy-5-(1-methyl-1H-benzo[d][1,2,3]triazol-6-yl)-7H-pyrrolo [2,3-d]pyrimidin-2-yl)amino)cyclohexan-1-ol 2197.

White solid (47.65 mg, 0.117 mmol). ¹H NMR (400 MHz, DMSO-d₆) δppm 1.37 (3 H, t, J=7.07 Hz), 1.44-1.56 (2 H, m), 1.59-1.79 (6 H, m), 3.74 (2 H, br s), 4.29 (3 H, s), 4.33 (1 H, br s), 4.45 (2 H, q, J=7.00 Hz), 6.39 (1 H, br d, J=7.50 Hz), 7.25 (1 H, d, J=2.25 Hz), 7.72 (1 H, dd, J=8.82, 1.06 Hz), 7.93 (1 H, d, J=8.75 Hz), 8.09 (1 H, s), 11.48 (1 H, br s); ESIMS found for C2J⁻125N702 m/z 408.1 (M+1).

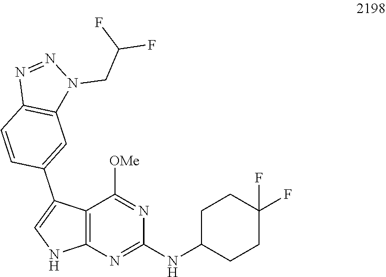 [SMILES: COc1nc(NC2CCC(F)(F)CC2)nc2[nH]cc(-c3ccc4nnn(CC(F)F)c4c3)c12]

N-(4,4-Difluorocyclohexyl)-5-(1-(2,2-difluoroethyl)-1H-benzo[d] [1,2,3]triazol-6-yl)-4-methoxy-7H-pyrrolo [2,3-d]pyrimidin-2-amine 2198.

White solid (4.8 mg, 0.010 mmol, 14.6% yield). ¹H NMR (499 MHz, CHLOROFORM-d) δppm 1.64-1.76 (2 H, m), 1.88-2.05 (2 H, m), 2.18 (4 H, br d, J=8.21 Hz), 4.82-4.90 (1 H, m), 5.01 (2 H, td, J=13.28, 3.56 Hz), 6.26 (1 H, t, J=55.12 Hz), 7.01 (1 H, br s), 7.67 (1 H, br d, J=8.49 Hz), 7.91 (1 H, s), 8.05 (1 H, br d, J=8.49 Hz), 8.58 (1 H, br s); ESIMS found for C2J⁻121F4N70 m/z 464.2 (M+1).

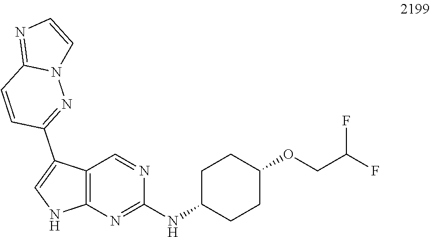 [SMILES: FC(F)CO[C@H]1CC[C@@H](Nc2ncc3c(-c4ccc5nccn5n4)c[nH]c3n2)CC1]

N-(cis-4-(2,2-Difluoroethoxy)cyclohexyl)-5-(imidazo [1,2-b] pyridazin-6-yl)-7H-pyrrolo [2,3-d]pyrimidin-2-amine 2199.

White solid (74.25 mg, 0.180 mmol). ¹H NMR (400 MHz, DMSO-d₆) δppm 1.49-1.58 (2 H, m), 1.58-1.66 (2 H, m), 1.66-1.74 (2 H, m), 1.81-1.90 (2 H, m), 3.56-3.61 (1 H, m), 3.67 (3 H, td, J=15.23, 3.81 Hz), 3.76-3.85 (1 H, m), 6.13 (1 H, tt, J=55.12, 3.76 Hz), 6.80 (1 H, br d, J=6.63 Hz), 7.68 (1 H, s), 7.73 (1 H, d, J=9.63 Hz), 8.03 (1 H, br d, J=12.01 Hz), 8.03 (1 H, s), 8.29 (1 H, s), 9.26 (1 H, s), 11.78 (1 H, br s); ESIMS found for C₂₀I-1₂₁F₂N₇O m/z 414.2 (M+1).

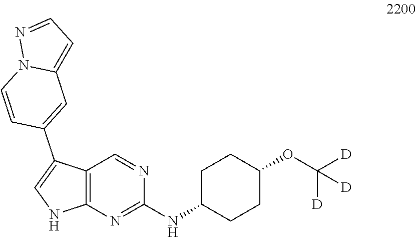 [SMILES: [2H]C([2H])([2H])O[C@H]1CC[C@@H](Nc2ncc3c(-c4ccn5nccc5c4)c[nH]c3n2)CC1]

N-(cis-4-(Methoxy-d₃)cyclohexyl)-5-(pyrazolo[1,5-a]pyridin-5-yl)-7H-pyrrolo[2,3-d]pyrimidin-2-amine 2200.

White solid (3.34 mg, 0.009 mmol). ¹H NMR (400 MHz, DMSO-d₆) δppm 1.43-1.52 (2 H, m), 1.54-1.64 (2 H, m), 1.65-1.73 (2 H, m), 1.80-1.91 (2 H, m), 3.33-3.38 (1 H, m), 3.73-3.85 (1 H, m), 6.55 (1 H, dd, J=2.20, 0.61 Hz), 6.71 (1 H, br d, J=7.70 Hz), 7.24 (1 H, dd, J=7.34, 1.96 Hz), 7.66 (1 H, d, J=2.45 Hz), 7.94 (1 H, d, J=2.20 Hz), 7.96 (1 H, d, J=1.34 Hz), 8.61 (1 H, d, J=7.34 Hz), 9.02 (1 H, s), 11.60 (1 H, br s); ESIMS found for C₂₀H₁₉[2H₃]N₆O m/z 366.4 (M+1).

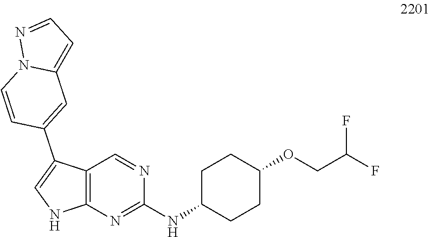 [SMILES: FC(F)CO[C@H]1CC[C@@H](Nc2ncc3c(-c4ccn5nccc5c4)c[nH]c3n2)CC1]

N-(cis-4-(2,2-Difluoroethoxy)cyclohexyl)-5-(pyrazolo[1,5-a]pyridin-5-yl)-7H-pyrrolo [2,3-d]pyrimidin-2-amine 2201.

White solid (45.48 mg, 0.110 mmol). ¹H NMR (400 MHz, DMSO-d₆) δppm 1.48-1.59 (2 H, m), 1.63 (2 H, br d, J=10.76 Hz), 1.65-1.74 (2 H, m), 1.80-1.93 (2 H, m), 3.58 (1 H, br s), 3.67 (2 H, td, J=15.20, 3.75 Hz), 3.75-3.85 (1 H, m), 6.12 (1 H, tt, J=55.20, 3.80 Hz), 6.55 (1 H, d, J=1.13 Hz), 6.72 (1 H, br d, J=7.38 Hz), 7.24 (1 H, d, J=7.50 Hz), 7.65 (1 H, s), 7.93 (1 H, d, J=1.38 Hz), 7.96 (1 H, s), 8.60 (1 H, d, J=7.38 Hz), 9.02 (1 H, s), 11.59 (1 H, br s); ESIMS found for C2J⁻122F2N60 m/z 413.1 (M+1).

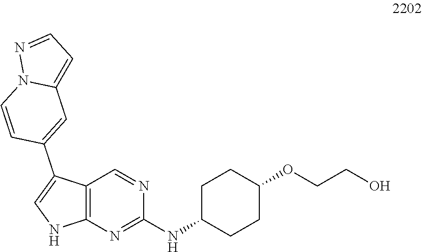 [SMILES: OCCO[C@H]1CC[C@@H](Nc2ncc3c(-c4ccn5nccc5c4)c[nH]c3n2)CC1]

2-((cis-4-((5-(Pyrazolo [1,5-a]pyridin-5-yl)-7H-pyrrolo [2,3-d] pyrimidin-2-yl)amino)cyclohexyl)oxy)ethan-1-ol 2202.

White solid (34.22 mg, 0.087 mmol). ¹H NMR (400 MHz, DMSO-d₆) δppm 1.44-1.56 (2 H, m), 1.60-1.73 (4 H, m), 1.80-1.89 (2 H, m), 3.40(2 H, t, J=5.36 Hz), 3.45-3.49 (1 H, m), 3.51 (2 H, q, J=5.36 Hz), 3.73-3.86 (1 H, m), 4.51 (1 H, t, J=5.50 Hz), 6.55 (1 H, d, J=2.00 Hz), 6.64-6.72 (1 H, m), 7.24 (1 H, dd, J=7.32, 1.81 Hz), 7.66 (1 H, d, J=2.25 Hz), 7.94 (1 H, d, J=2.13 Hz), 7.96 (1 H, s), 8.61 (1 H, d, J=7.50 Hz), 9.02 (1 H, s), 11.59 (1 H, br s); ESIMS found for C2J⁻124N602 m/z 393.2 (M+1).

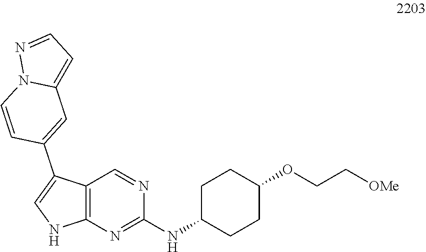 [SMILES: COCCO[C@H]1CC[C@@H](Nc2ncc3c(-c4ccn5nccc5c4)c[nH]c3n2)CC1]

N-(cis-4-(2-Methoxyethoxy)cyclohexyl)-5-(pyrazolo [1,5-a]pyridin-5-yl)-7H-pyrrolo [2,3-d] pyrimidin-2-amine 2203.

Beige solid (17 mg, 0.042 mmol, 32.7% yield). ¹H NMR (499 MHz, DMSO-d₆) δppm 1.44-1.55 (2 H, m), 1.57-1.74 (4 H, m), 1.78-1.89 (2 H, m), 3.27 (3 H, s), 3.44-3.47 (2 H, m), 3.45-3.51 (1 H, m), 3.48-3.52 (2 H, m), 3.71-3.84 (1 H, m), 6.55 (1 H, d, J=1.64 Hz), 6.72 (1 H, br d, J=6.57 Hz), 7.19-7.28 (1 H, m), 7.66 (1 H, d, J=2.19 Hz), 7.94 (1 H, d, J=2.19 Hz), 7.96 (1 H, d, J=1.10 Hz), 8.61 (1 H, d, J=7.12 Hz), 9.02 (1 H, s), 11.60 (1 H, br s); ESIMS found for C22H₂₆N602 m/z 407.2 (M+1).

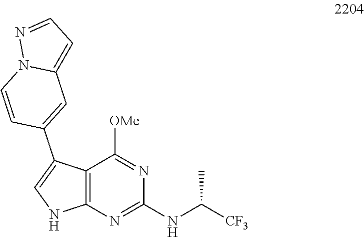 [SMILES: COc1nc(N[C@H](C)C(F)(F)F)nc2[nH]cc(-c3ccn4nccc4c3)c12]

(R)-4-Methoxy-5-(pyrazolo [1,5-a]pyridin-5-yl)-N-(1, 1, 1-trifluoropropan-2-yl)-7H-pyrrolo [2,3-d] pyrimidin-2-amine 2204.

White solid (98.11 mg, 0.261 mmol). ¹H NMR (400 MHz, DMSO-d₆) δppm 1.36 (3 H, br d, J=6.88 Hz), 4.01 (3 H, s), 4.94 (1 H, br dd, J=14.88, 7.38 Hz), 6.55 (1 H, s), 7.13-7.31 (2 H, m), 7.42 (1 H, s), 7.88-8.05 (2 H, m), 8.59 (1 H, br d, J=7.25 Hz), 11.69 (1 H, br s); ESIMS found for C₁₇H₁₅F₃N₆O m/z 377.1 (M+1).

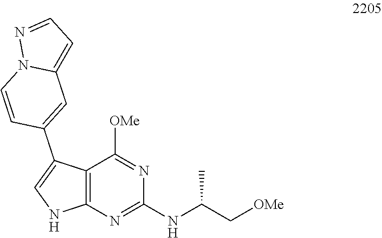 [SMILES: COC[C@@H](C)Nc1nc(OC)c2c(-c3ccn4nccc4c3)c[nH]c2n1]

(R)-4-Methoxy-N-(1-methoxypropan-2-yl)-5-(pyrazolo [1,5-a]pyridin-5-yl)-7H-pyrrolo [2,3-d]pyrimidin-2-amine 2205.

White solid (82.16 mg, 0.233 mmol). ¹H NMR (400 MHz, DMSO-d₆) δppm 1.16 (3 H, d, J=6.63 Hz), 3.23-3.29 (1 H, m), 3.28 (3 H, s), 3.46 (1 H, br dd, J=9.26, 5.63 Hz), 3.97 (3 H, s), 4.18 (1 H, dquin, J=13.56, 6.62, 6.62, 6.62, 6.62 Hz), 6.42 (1 H, d, J=8.25 Hz), 6.54 (1 H, d, J=1.63 Hz), 7.18 (1 H, dd, J=7.38, 1.75 Hz), 7.34 (1 H, d, J=2.00 Hz), 7.93 (1 H, d, J=2.13 Hz), 7.99 (1 H, s), 8.57 (1 H, d, J=7.38 Hz), 11.54 (1 H, br s); ESIMS found for C181⁻120N602 m/z 353. (M+1).

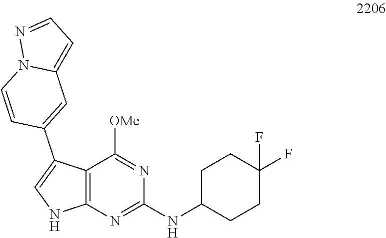 [SMILES: COc1nc(NC2CCC(F)(F)CC2)nc2[nH]cc(-c3ccn4nccc4c3)c12]

N-(4,4-Difluorocyclohexyl)-4-methoxy-5-(pyrazolo[1,5-a]pyridin-5-yl)-7H-pyrrolo [2,3-d] pyrimidin-2-amine 2206.

White solid (770.8 mg, 1.936 mmol). ¹H NMR (400 MHz, DMSO-d₆) δppm 1.56-1.72 (2 H, m), 1.82-2.02 (4 H, m), 2.04-2.15 (2 H, m), 3.31 (3 H, s), 3.86-3.96 (1 H, m), 3.98 (3 H, s), 6.54 (1 H, d, J=1.75 Hz), 6.72 (1 H, d, J=7.75 Hz), 7.18 (1 H, dd, J=7.32, 1.81 Hz), 7.34 (1 H, s), 7.93 (1 H, d, J=2.13 Hz), 7.99 (1 H, s), 8.58 (1 H, d, J=7.25 Hz), 11.55 (1 H, br s); ESIMS found for C₂₀1⁻1₂₀F₂N₆O m/z 399.2 (M+1).

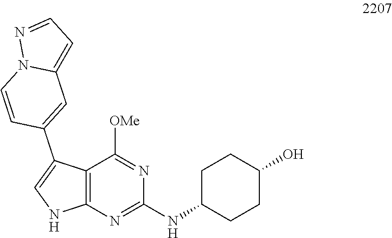 [SMILES: COc1nc(N[C@H]2CC[C@@H](O)CC2)nc2[nH]cc(-c3ccn4nccc4c3)c12]

cis-4-((4-Methoxy-5-(pyrazolo [1,5-a]pyridin-5-yl)-7H-pyrrolo [2,3-d]pyrimidin-2-yl)amino)cyclohexan-1-ol 2207.

Off-white solid (31 mg, 0.082 mmol, 43.5% yield). ¹1-1 NMR (499 MHz, DMSO-d₆) δppm 1.44-1.55 (2 H, m), 1.60-1.69 (4 H, m), 1.69-1.77 (2 H, m), 3.70-3.80 (2 H, m), 3.97(3 H, s), 4.33(1 H, d, J=3.01 Hz), 6.45 (1 H, d, J=7.67 Hz), 6.52-6.57 (1 H, m), 7.18 (1 H, dd, J=7.39, 1.92 Hz), 7.32 (1 H, d, J=1.64 Hz), 7.92 (1 H, d, J=2.19 Hz), 7.99 (1 H, d, J=1.09 Hz), 8.57 (1 H, d, J=7.39 Hz), 11.52 (1 H, br s); ESIMS found for C₂oH₂₂N₆O₂ m/z 379.2 (M+1).

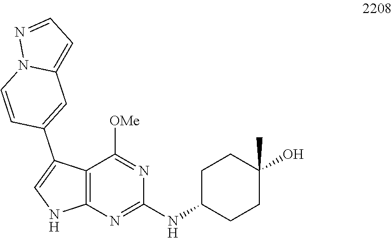 [SMILES: COc1nc(N[C@H]2CC[C@@](C)(O)CC2)nc2[nH]cc(-c3ccn4nccc4c3)c12]

cis-4-((4-Methoxy-5-(pyrazolo [1,5-a]pyridin-5-yl)-7H-pyrrolo [2,3-d]pyrimidin-2-yl)amino)-1-methylcyclohexan-1-ol 2208.

White solid (12 mg, 0.031 mmol, 33.4% yield). ¹H NMR (499 MHz, DMSO-d₆) δppm 1.12 (3 H, s), 1.35 (2 H, td, J=12.94, 4.24 Hz), 1.58 (2 H, br d, J=12.05 Hz), 1.61-1.72 (4 H, m), 3.60-3.72 (1 H, m), 3.97 (3 H, s), 3.99 (1 H, s), 6.45 (1 H, d, J=7.94 Hz), 6.54 (1 H, d, J=2.19 Hz), 7.18 (1 H, dd, J=7.39, 1.92 Hz), 7.31 (1 H, d, J=2.19 Hz), 7.92 (1 H, d, J=2.19 Hz), 7.99 (1 H, d, J=0.82 Hz), 8.57 (1 H, d, J=7.39 Hz), 11.51 (1 H, br s); ESIMS found for C_{2I}F1₂₄N₆O₂ m/z 393.2 (M+1).

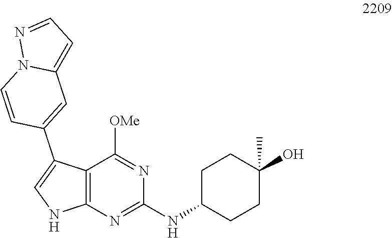 [SMILES: COc1nc(N[C@H]2CC[C@](C)(O)CC2)nc2[nH]cc(-c3ccn4nccc4c3)c12]

trans-4-44-Methoxy-5-(pyrazolo [1,5-a] pyridin-5-yl)-7H-pyrrolo [2,3-d] pyrimidin-2-yl)amino)-1-methylcyclohexan-1-ol 2209.

Beige solid (57 mg, 0.145 mmol, 52.9% yield). ¹H NMR (499 MHz, DMSO-d₆) δppm 1.15 (3 H, s), 1.37-1.51 (4 H, m), 1.53-1.66 (2 H, m), 1.81-1.91 (2 H, m), 3.73-3.85 (1 H, m), 3.97 (3 H, s), 4.22 (1 H, s), 6.45 (1 H, d, J=8.21 Hz), 6.54 (1 H, d, J=1.64 Hz), 7.18 (1 H, dd, J=7.39, 1.92 Hz), 7.32 (1 H, d, J=2.19 Hz), 7.92 (1 H, d, J=2.19 Hz), 7.98 (1 H, d, J=1.10 Hz), 8.57 (1 H, d, J=7.39 Hz), 11.53 (1 H, br s); ESIMS found for C_{2I}F1₂₄N₆O₂ m/z 393.25 (M+1).

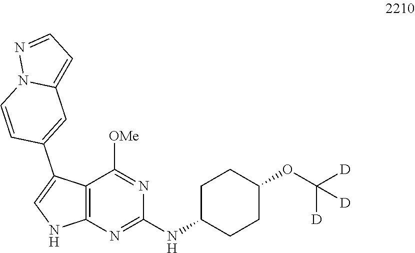 [SMILES: [2H]C([2H])([2H])O[C@H]1CC[C@@H](Nc2nc(OC)c3c(-c4ccn5nccc5c4)c[nH]c3n2)CC1]

4-Methoxy-N-(cis-4-(methoxy-d₃)cyclohexyl)-5-(pyrazolo [1,5-a] pyridin-5-yl)-7H-pyrrolo [2,3-d] pyrimidin-2-amine 2210.

Beige solid (95 mg, 0.240 mmol, 80.5% yield). ¹H NMR (499 MHz, DMSO-d₆) δppm 1.44-1.53 (2 H, m), 1.55-1.64 (2 H, m), 1.65-1.72 (2 H, m), 1.81-1.89 (2 H, m), 3.33-3.37 (1 H, m), 3.72-3.83 (1 H, m), 3.97 (3 H, s), 6.52 (1 H, d, J=7.67 Hz), 6.54 (1 H, dd, J=2.19, 0.82 Hz), 7.18 (1 H, dd, J=7.39, 1.92 Hz), 7.32 (1 H, d, J=2.46 Hz), 7.92 (1 H, d, J=2.19 Hz), 7.99 (1 H, d, J=1.37 Hz), 8.57 (1 H, d, J=7.39 Hz), 11.52 (1 H, br s); ESIMS found for C_{2I}F1₂₁[2H₃]N₆O₂ m/z 396.2 (M+1).

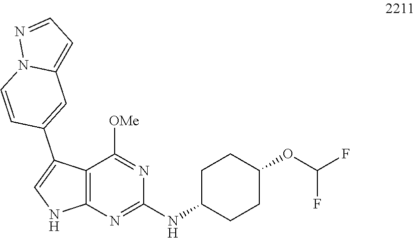 [SMILES: COc1nc(N[C@H]2CC[C@@H](OC(F)F)CC2)nc2[nH]cc(-c3ccn4nccc4c3)c12]

N-(cis-4-(Difluoromethoxy)cyclohexyl)-4-methoxy-5-(pyrazolo [1,5-a] pyridin-5-yl)-7H-pyrrolo [2,3-d]pyrimidin-2-amine 2211.

Off-white solid (31 mg, 0.072 mmol, 42.2% yield). ¹H NMR (499 MHz, DMSO-d₆) δppm 1.61-1.72 (4 H, m), 1.74-1.82 (2 H, m), 1.84-1.93 (2 H, m), 3.77-3.86 (1 H, m), 3.98 (3 H, s), 4.24-4.33 (1 H, m), 6.53-6.55 (1 H, m), 6.72 (2 H, t, J=77.05 Hz), 6.64 (1 H, d, J=7.67 Hz), 7.18 (1 H, dd, J=7.39, 1.92 Hz), 7.33 (1 H, d, J=2.46 Hz), 7.93 (1 H, d, J=2.46 Hz), 7.99 (1 H, d, J=1.37 Hz), 8.57 (1 H, d, J=7.12 Hz); ESIMS found for C_{2I}F1₂₂F₂N₆O₂ m/z 429.2 (M+1).

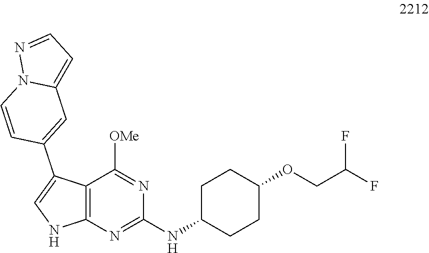 [SMILES: COc1nc(N[C@H]2CC[C@@H](OCC(F)F)CC2)nc2[nH]cc(-c3ccn4nccc4c3)c12]

N-(cis-4-(2,2-Difluoroethoxy)cyclohexyl)-4-methoxy-5-(pyrazolo [1,5-a] pyridin-5-yl)-7H-pyrrolo [2,3-d]pyrimidin-2-amine 2212.

Beige solid (109 mg, 0.246 mmol, 73.2% yield). ¹H NMR (499 MHz, DMSO-d₆) δppm 1.49-1.57 (2 H, m), 1.59-1.67 (2 H, m), 1.67-1.73 (2 H, m), 1.82-1.89 (2 H, m), 3.56 -3.61 (1 H, m), 3.67 (2 H, td, J=15.19, 3.83 Hz), 3.75-3.83 (1 H, m), 3.97 (3 H, s), 6.13 (1 H, tt, J=55.10, 3.85 Hz), 6.52-6.58 (2 H, m), 7.18 (1 H, dd, J=7.26, 2.05 Hz), 7.32 (1 H, d, J=1.92 Hz), 7.92 (1 H, d, J=2.19 Hz), 7.99 (1 H, d, J=1.09 Hz), 8.57 (1 H, d, J=7.39 Hz), 11.52 (1 H, br s); ESIMS found for C22H₂₄F2N602 m/z 443.2 (M+1).

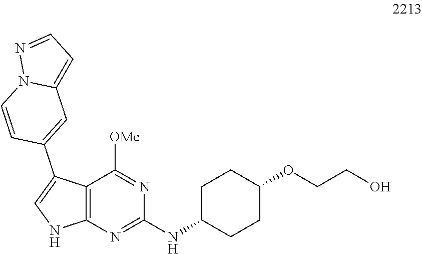 [SMILES: COc1nc(N[C@H]2CC[C@@H](OCCO)CC2)nc2[nH]cc(-c3ccn4nccc4c3)c12]

2-((cis-4-((4-Methoxy-5-(pyrazolo[1,5-a]pyridin-5-yl)-7H-pyrrolo[2,3-d]pyrimidin-2-yl)amino)cyclohexyl)oxy)ethan-1-ol 2213.

Beige solid (3.2 mg, 0.008 mmol, 11.1% yield). ¹H NMR (499 MHz, DMSO-d₆) δppm 1.46-1.54 (2 H, m), 1.62-1.70 (4 H, m), 1.80-1.88 (2 H, m), 3.40 (2 H, t, J=5.50 Hz), 3.48 (1 H, br d, J=3.01 Hz), 3.51 (2 H, q, J=5.48 Hz), 3.74-3.82 (1 H, m), 3.97 (3 H, s), 4.51 (1 H, t, J=5.61 Hz), 6.48 (1 H, d, J=7.67 Hz), 6.54 (1 H, d, J=1.64 Hz), 7.18 (1 H, dd, J=7.26, 2.05 Hz), 7.32 (1 H, d, J=2.19 Hz), 7.92 (1 H, d, J=2.19 Hz), 7.99 (1 H, s), 8.57 (1 H, d, J=7.39 Hz), 11.52 (1 H, br s); ESIMS found for C₂₂H₂₆N₆O₃ m/z 423.2 (M+1).

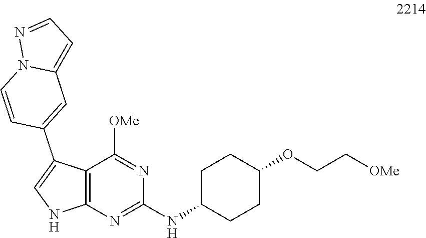 [SMILES: COCCO[C@H]1CC[C@@H](Nc2nc(OC)c3c(-c4ccn5nccc5c4)c[nH]c3n2)CC1]

4-Methoxy-N-(cis-4-(2-methoxyethoxy)cyclohexyl)-5-(pyrazolo[1,5-a] pyridin-5-yl)-7H-pyrrolo [2,3-d] pyrimidin-2-amine 2214.

Beige solid (31 mg, 0.071 mmol, 44.2% yield). ¹H NMR (499 MHz, DMSO-d₆) δppm 1.44-1.54 (2 H, m), 1.59-1.74 (4 H, m), 1.79-1.88 (2 H, m), 3.44-3.47 (2 H, m), 3.47 -3.48 (1 H, m), 3.49-3.52 (2 H, m), 3.72-3.82 (1 H, m), 3.97 (3 H, s), 6.51-6.55 (2 H, m), 7.18 (1 H, dd, J=7.26, 2.05 Hz), 7.32 (1 H, d, J=2.46 Hz), 7.92 (1 H, d, J=2.19 Hz), 7.99 (1 H, d, J=1.37 Hz), 8.57 (1 H, d, J=7.12 Hz), 11.52 (1 H, br s); ESIMS found for C₂₃H₂₈N₆O₃ m/z 437.3 (M+1).

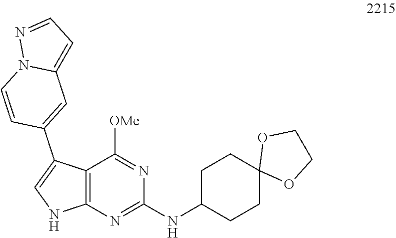 [SMILES: COc1nc(NC2CCC3(CC2)OCCO3)nc2[nH]cc(-c3ccn4nccc4c3)c12]

4-Methoxy-5-(pyrazolo [1,5-a]pyridin-5-yl)-N-(1,4-dioxaspiro[4.5] de can-8-yl)-7H-pyrrolo[2,3-d]pyrimidin-2-amine 2215.

Off-white solid (25 mg, 0.060 mmol, 32.2% yield). ¹H NMR (499 MHz, DMSO-d₆) δppm 1.51-1.63 (4 H, m), 1.73 (2 H, br dd, J=8.62, 2.87 Hz), 1.87-1.93 (2 H, m), 3.76-3.83 (1 H, m), 3.84-3.90 (4 H, m), 3.97 (3 H, s), 6.54 (1 H, d, J=1.64 Hz), 6.60 (1 H, d, J=7.67 Hz), 7.18 (1 H, dd, J=7.39, 1.92 Hz), 7.32 (1 H, d, J=1.92 Hz), 7.93 (1 H, d, J=2.19 Hz), 7.98 (1 H, d, J=1.10 Hz), 8.57 (1 H, d, J=7.12 Hz), 11.55 (1 H, br s); ESIMS found for C₂₂H₂₄N₆O₃ m/z 421.2 (M+1).

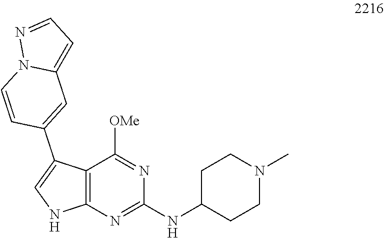 [SMILES: COc1nc(NC2CCN(C)CC2)nc2[nH]cc(-c3ccn4nccc4c3)c12]

4-Methoxy-N-(1-methylpiperidin-4-yl)-5-(pyrazolo [1,5-a] pyridin-5-yl)-7H-pyrrolo [2,3-d] pyrimidin-2-amine 2216.

White solid (8.50 mg, 0.023 mmol). ¹H NMR (400 MHz, DMSO-d₆) δppm 1.49-1.66 (2 H, m), 1.91 (2 H, br d, J=10.27 Hz), 2.14 (2 H, br t, J=10.94 Hz), 2.26 (3 H, s), 2.86 (2 H, br d, J=11.86 Hz), 3.68-3.82 (1 H, m), 3.97 (3 H, s), 6.54 (1 H, d, J=1.59 Hz), 6.60 (1 H, d, J=7.82 Hz), 7.18 (1 H, dd, J=7.34, 1.83 Hz), 7.33 (1 H, d, J=2.20 Hz), 7.93 (1 H, d, J=2.20 Hz), 7.98 (1 H, d, J=0.73 Hz), 8.57 (1 H, d, J=7.34 Hz), 11.55 (1 H, br s); ESIMS found for C₂₀H₂₃N₇O m/z 378.1 (M+1).

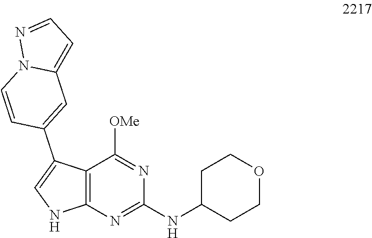 [SMILES: COc1nc(NC2CCOCC2)nc2[nH]cc(-c3ccn4nccc4c3)c12]

4-Methoxy-5-(pyrazolo [1,5-a] pyridin-5-yl)-N-(tetrahydro-2H-pyran-4-yl)-7H-pyrrolo [2,3-d] pyrimidin-2-amine 2217.

White solid (27.25 mg, 0.075 mmol). ¹H NMR (400 MHz, DMSO-d₆) δppm 1.45-1.63 (2 H, m), 1.88 (2 H, br dd, J=12.47, 1.96 Hz), 3.33-3.44 (2 H, m), 3.89 (2 H, br d, J=10.88 Hz), 3.92-3.96 (1 H, m), 3.98 (3 H, s), 6.54 (1 H, d, J=1.83 Hz), 6.66 (1 H, d, J=7.82 Hz), 7.18 (1 H, dd, J=7.34, 1.83 Hz), 7.34 (1 H, d, J=2.32 Hz), 7.93 (1 H, d, J=2.20 Hz), 7.99 (1 H, d, J=0.98 Hz), 8.58 (1 H, d, J=7.34 Hz), 11.57 (1 H, br s); ESIMS found for C₁₉H₂₀N₆O₂ m/z 365.1 (M+1).

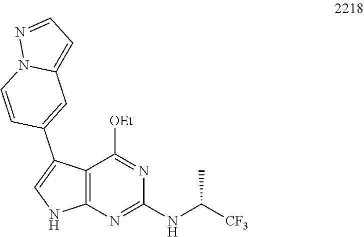 [SMILES: CCOc1nc(N[C@H](C)C(F)(F)F)nc2[nH]cc(-c3ccn4nccc4c3)c12]

(R)-4-Ethoxy-5-(pyrazolo[1,5-a]pyridin-5-yl)-N-(1,1,1-trifluoropropan-2-yl)-7H-pyrrolo[2,3-d]pyrimidin-2-amine 2218.

White solid (46.27 mg, 0.119 mmol). ¹H NMR (400 MHz, DMSO-d₆) δppm 1.35 (3 H, d, J=7.00 Hz), 1.40 (3 H, t, J=7.07 Hz), 4.48 (2 H, q, J=7.09 Hz), 4.91 (1 H, dq, J=15.49, 7.76 Hz), 6.52 (1 H, d, J=1.88 Hz), 7.19-7.24 (2 H, m), 7.42 (1 H, d, J=2.38 Hz), 7.93 (1 H, d, J=2.13 Hz), 8.07 (1 H, d, J=1.13 Hz), 8.59 (1 H, d, J=7.38 Hz), 11.64 (1 H, br s); ESIMS found for C₁₈H₁₇F3N60 m/z 391.0 (M+1).

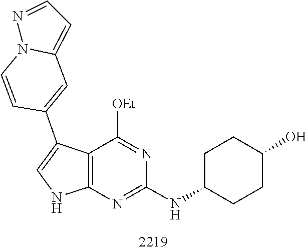 [SMILES: CCOc1nc(N[C@H]2CC[C@@H](O)CC2)nc2[nH]cc(-c3ccn4nccc4c3)c12]

cis-4-((4-Ethoxy-5-(pyrazolo [1,5-a]pyridin-5-yl)-7H-pyrrolo [2,3-d]pyrimidin-2-yl)amino)cyclohexan-1-ol 2219.

White solid (35.09 mg, 0.089 mmol). ¹H NMR (400 MHz, DMSO-d₆) δppm 1.39 (3 H, t, J=7.07 Hz), 1.45-1.55 (2 H, m), 1.59-1.77 (6 H, m), 3.73 (2 H, br s), 4.34 (1 H, d, J=3.00 Hz), 4.45 (2 H, q, J=7.00 Hz), 6.40 (1 H, d, J=7.63 Hz), 6.51 (1 H, d, J=1.75 Hz), 7.20 (1 H, dd, J=7.38, 1.88 Hz), 7.33 (1 H, d, J=2.38 Hz), 7.92 (1 H, d, J=2.25 Hz), 8.08 (1 H, d, J=0.88 Hz), 8.57 (1 H, d, J=7.25 Hz), 11.50 (1 H, br s); ESIMS found for C211⁻124N602 m/z 393.1 (M+1).

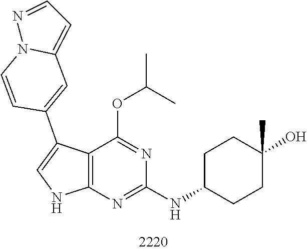 [SMILES: CC(C)Oc1nc(N[C@H]2CC[C@@](C)(O)CC2)nc2[nH]cc(-c3ccn4nccc4c3)c12]

cis-4-((4-Isopropoxy-5-(pyrazolo [1,5-a] pyridin-5-yl)-7H-pyrrolo [2,3-d]pyrimidin-2-yl)amino)-1-methylcyclohexan-1-ol 2220.

Off-white solid (27 mg, 0.064 mmol, 20.8% yield). ¹1-1 NMR (499 MHz, DMSO-d₆) δppm 1.15 (3 H, s), 1.37 (6 H, d, J=6.02 Hz), 1.40-1.48 (4 H, m), 1.56-1.64 (2 H, m), 1.81-1.90 (2 H, m), 3.70-3.82 (1 H, m), 4.23 (1 H, s), 5.46 (1 H, dquin, J=12.41, 6.14, 6.14, 6.14, 6.14 Hz), 6.38 (1 H, d, J=7.94 Hz), 6.50 (1 H, d, J=1.64 Hz), 7.20 (1 H, dd, J=7.39, 1.92 Hz), 7.32 (1 H, d, J=1.64 Hz), 7.92 (1 H, d, J=2.19 Hz), 8.08 (1 H, d, J=1.10 Hz), 8.57 (1 H, d, J=7.12 Hz), 11.48 (1 H, br s); ESIMS found for C₂₃H₂₈N₆O₂ m/z 421.2 (M+1).

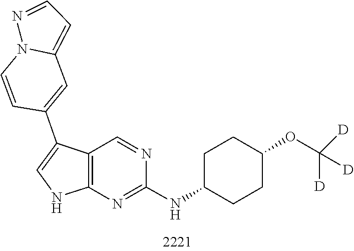 [SMILES: [2H]C([2H])([2H])O[C@H]1CC[C@@H](Nc2ncc3c(-c4ccn5nccc5c4)c[nH]c3n2)CC1]

N-(cis-4-(Methoxy-d₃)cyclohexyl)-5-(pyrazolo [1,5-a] pyrimidin-5-yl)-7H-pyrrolo [2,3-d] pyrimidin-2-amine 2221.

White solid (48.56 mg, 0.133 mmol). ¹H NMR (400 MHz, DMSO-d₆) δppm 1.43-1.53 (2 H, m), 1.53-1.64 (2 H, m), 1.65-1.72 (2 H, m), 1.80-1.90 (2 H, m), 3.32-3.38 (1 H, m), 3.73-3.85 (1 H, m), 6.59 (1 H, d, J=2.00 Hz), 6.79 (1 H, br d, J=6.50 Hz), 7.46 (1 H, d, J=7.38 Hz), 8.10 (2 H, t, J=2.38 Hz), 8.95 (1 H, d, J=7.38 Hz), 9.32 (1 H, s), 11.85 (1 H, br s); ESIMS found for C₁₉H₁₈[2H₃]1\1₇0 m/z 367.5 (M+1).

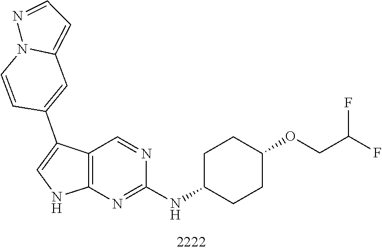 [SMILES: FC(F)CO[C@H]1CC[C@@H](Nc2ncc3c(-c4ccn5nccc5c4)c[nH]c3n2)CC1]

N-(cis-4-(2,2-Difluoroethoxy)cyclohexyl)-5-(pyrazolo [1,5-a]pyrimidin-5-yl)-7H-pyrrolo [2,3-d]pyrimidin-2-amine 2222.

White solid (66.9 mg, 0.162 mmol). ¹H NMR (400 MHz, DMSO-d₆) δppm 1.49-1.59 (2 H, m), 1.63 (2 H, br d, J=11.26 Hz), 1.66-1.74 (2 H, m), 1.78-1.94 (2 H, m), 3.56-3.61 (1 H, m), 3.67 (2 H, td, J=15.20, 3.88 Hz), 3.75-3.85 (1 H, m), 6.13 (1 H, tt, J=55.12, 3.72 Hz), 6.59 (1 H, d, J=1.88 Hz), 6.82 (1 H, br d, J=5.75 Hz), 7.46 (1 H, d, J=7.38 Hz), 8.10 (2 H, t, J=2.63 Hz), 8.95 (1 H, d, J=7.38 Hz), 9.32 (1 H, s), 11.85 (1 H, br s); ESIMS found for C₂₀I-1₂₁F₂N₇O m/z 414.1 (M+1).

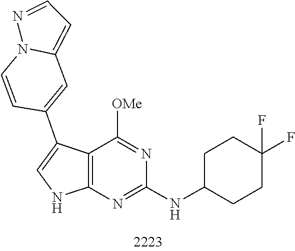 [SMILES: COc1nc(NC2CCC(F)(F)CC2)nc2[nH]cc(-c3ccn4nccc4c3)c12]

N-(4,4-Difluorocyclohexyl)-4-methoxy-5-(pyrazolo[1,5-a]pyrimidin-5-yl)-7H-pyrrolo[2,3-d]pyrimidin-2-amine 2223.

White solid (9.54 mg, 0.024 mmol). ¹H NMR (400 MHz, DMSO-d₆) δppm 1.54-1.74 (2 H, m), 1.80-2.03 (4 H, m), 2.04-2.19 (2 H, m), 3.86-3.98 (1 H, m), 4.01 (3 H, s), 6.53 (1 H, dd, J=2.20, 0.73 Hz), 6.81 (1 H, d, J=7.70 Hz), 7.63 (1 H, s), 7.67 (1 H, d, J=7.46 Hz), 8.12 (1 H, d, J=2.20 Hz), 8.97 (1 H, dd, J=7.46, 0.73 Hz), 11.72 (1 H, br s); ESIMS found for C₁₉H₁₉F2N70 m/z 400.1 (M+1).

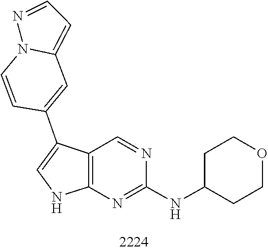 [SMILES: c1cc2cc(-c3c[nH]c4nc(NC5CCOCC5)ncc34)ccn2n1]

4-Methoxy-5-(pyrazolo[1,5-a]pyrimidin-5-yl)-N-(tetrahydro-2H-pyran-4-yl)-7H-pyrrolo [2,3-d]pyrimidin-2-amine 2224.

White solid (2.16 mg, 0.006 mmol). ¹H NMR (400 MHz, DMSO-d₆) δppm 1.46-1.63 (2 H, m), 1.88 (2 H, br d, J=12.26 Hz), 3.37-3.44 (2 H, m), 3.89 (2 H, br d, J=10.76 Hz), 3.92-3.99 (1 H, m), 4.01 (3 H, s), 6.53 (1 H, d, J=1.63 Hz), 6.76 (1 H, d, J=7.88 Hz), 7.62 (1 H, s), 7.67(1 H, d, J=7.38 Hz), 8.12 (1 H, d, J=2.25 Hz), 8.98 (1 H, d, J=7.38 Hz), 11.86 (1 H, br s); ESIMS found for C₁₈H₁₉N702 m/z 366.1 (M+1).

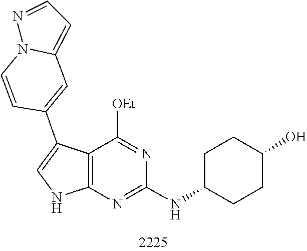 [SMILES: CCOc1nc(N[C@H]2CC[C@@H](O)CC2)nc2[nH]cc(-c3ccn4nccc4c3)c12]

cis-4-44-Ethoxy-5-(pyrazolo [1,5-a]pyrimidin-5-yl)-7H-pyrrolo [2,3-d]pyrimidin-2-yl)amino)cyclohexan-1-ol 2225.

White solid (21.85 mg, 0.056 mmol). ¹H NMR (400 MHz, DMSO-d₆) δppm 1.41 (3 H, br t, J=6.85 Hz), 1.45-1.55 (2 H, m), 1.58-1.78 (6 H, m), 3.74 (2 H, br s), 4.33 (1 H, br s), 4.48 (2 H, q, J=6.81 Hz), 6.44-6.57 (2 H, m), 7.59 (1 H, s), 7.71 (1 H, br d, J=7.46 Hz), 8.11 (1 H, s), 9.00 (1 H, br d, J=7.34 Hz), 11.78 (1 H, br s); ESIMS found for C₂₀H₂₃N₇O₂ m/z 394.1 (M+1).

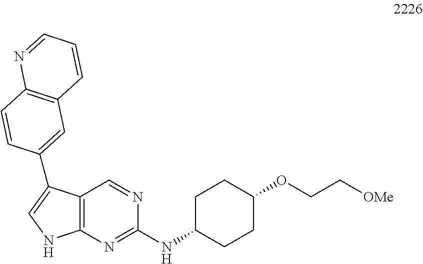 [SMILES: COCCO[C@H]1CC[C@@H](Nc2ncc3c(-c4ccc5ncccc5c4)c[nH]c3n2)CC1]

N-(cis-4-(2-Methoxyethoxy)cyclohexyl)-5-(quinolin-6-yl)-7H-pyrrolo[2,3-d]pyrimidin-2-amine 2226.

Off-white solid (16 mg, 0.038 mmol, 31.9% yield). ¹H NMR (499 MHz, DMSO-d₆) δppm 1.46-1.55 (2 H, m), 1.59-1.73 (4 H, m), 1.80-1.88 (2 H, m), 3.27 (3 H, s), 3.44-3.47 (2 H, m), 3.45-3.53 (1 H, m), 3.49-3.52 (2 H, m), 3.73-3.86 (1 H, m), 6.69 (1 H, br d, J=7.12 Hz), 7.51 (1 H, dd, J=8.21, 3.83 Hz), 7.66 (1 H, d, J=1.64 Hz), 7.99 (1 H, d, J=8.76 Hz), 8.14 (1 H, dd, J=8.76, 2.19 Hz), 8.28 (1 H, d, J=2.19 Hz), 8.40-8.48 (1 H, m), 8.81 (1 H, dd, J=4.38, 1.64 Hz), 9.12 (1 H, s), 11.57 (1 H, br s); ESIMS found for C₂₄H₂₇N₅O₂ m/z 418.2 (M+1).

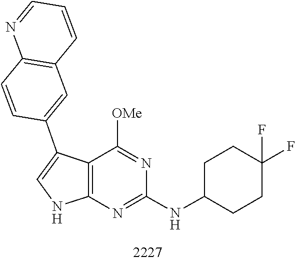 [SMILES: COc1nc(NC2CCC(F)(F)CC2)nc2[nH]cc(-c3ccc4ncccc4c3)c12]

N-(4,4-Difluorocyclohexyl)-4-methoxy-5-(quinolin-6-yl)-7H-pyrrolo[2,3-d]pyrimidin-2-amine 2227.

White solid (23.09 mg, 0.056 mmol). ¹H NMR (400 MHz, DMSO-d₆) δppm 1.57-1.72 (2 H, m), 1.82-2.03 (4 H, m), 2.04-2.18 (2 H, m), 3.90-3.97 (1 H, m), 3.99 (3 H, s), 6.80 (1 H, br s), 7.38 (1 H, d, J=2.13 Hz), 7.78 (1 H, dd, J=8.25, 4.75 Hz), 8.08 (1 H, d, J=8.88 Hz), 8.26 (1 H, dd, J=8.88, 1.50 Hz), 8.40 (1 H, d, J=1.13 Hz), 8.73 (1 H, br d, J=8.00 Hz), 9.01 (1 H, d, J=3.75 Hz), 11.67 (1 H, br s); ESIMS found for C₂₂H₂₁F₂N₅O m/z 410.1 (M+1).

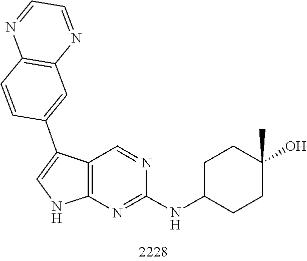

cis-1-Methyl-4-45-(quinoxalin-6-yl)-7H-pyrrolo[2,3-d]pyrimidin-2-ylamino)cyclohexan-1-ol 2228.

Yellow solid (15 mg, 0.040 mmol, 57.3% yield). ¹H NMR (499 MHz, DMSO-d₆) δppm 1.13 (3 H, s), 1.37 (2 H, td, J=12.73, 4.65 Hz), 1.59 (2 H, br d, J=12.05 Hz), 1.63-1.73 (4 H, m), 3.68 (1 H, br d, J=6.57 Hz), 4.00 (1 H, s), 6.61-6.71 (1 H, m), 7.78 (1 H, s), 8.08 (1 H, d, J=8.76 Hz), 8.24-8.32 (2 H, m), 8.85 (1 H, d, J=2.19 Hz), 8.92 (1 H, d, J=1.64 Hz), 9.01 (1 H, s), 11.70 (1 H, br s); ESIMS found for C₂₁1⁻1₂₂N₆O m/z 375.2 (M+1).

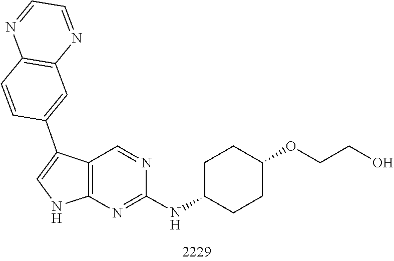 [SMILES: OCCO[C@H]1CC[C@@H](Nc2ncc3c(-c4ccc5nccnc5c4)c[nH]c3n2)CC1]

2-((cis-4-((5-(Quinoxalin-6-yl)-7H-pyrrolo [2,3-d]pyrimidin-2-yl)amino)cyclohexyl)oxy)ethan-1-ol 2229.

White solid (98.97 mg, 0.245 mmol). ¹H NMR (400 MHz, DMSO-d₆) δppm 1.47-1.55 (2 H, m), 1.62-1.72 (4 H, m), 1.80-1.89 (2 H, m), 3.41 (2 H, t, J=5.60 Hz), 3.46-3.49 (1 H, m), 3.51 (2 H, q, J=5.60 Hz), 3.75-3.86 (1 H, m), 4.51 (1 H, t, J=5.50 Hz), 6.68-6.76 (1 H, m), 7.79 (1 H, s), 8.08 (1 H, d, J=8.76 Hz), 8.24-8.30 (2 H, m), 8.85 (1 H, s), 8.92 (1 H, s), 9.02 (1 H, s), 11.71 (1 H, br s); ESIMS found for C₂₂H₂₄N₆O₂ m/z 405.3 (M+1).

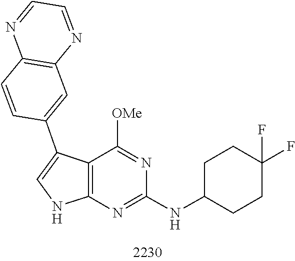 [SMILES: COc1nc(NC2CCC(F)(F)CC2)nc2[nH]cc(-c3ccc4nccnc4c3)c12]

N-(4,4-Difluorocyclohexyl)-4-methoxy-5-(quinoxalin-6-yl)-7H-pyrrolo [2,3-d]pyrimidin-2-amine 2230.

White solid (1.06 mg, 0.003 mmol). ¹H NMR (400 MHz, DMSO-d₆) δppm 1.58-1.73 (2 H, m), 1.82-2.03 (4 H, m), 2.05-2.16 (2 H, m), 3.88-3.97 (1 H, m), 3.98 (3 H, s), 6.73-6.80 (1 H, m), 7.44 (1 H, d, J=2.45 Hz), 8.02 (1 H, d, J=8.80 Hz), 8.19 (1 H, br dd, J=8.80, 1.96 Hz), 8.40 (1 H, d, J=1.96 Hz), 8.84 (1 H, d, J=1.83 Hz), 8.90 (1 H, d, J=1.83 Hz), 11.65 (1 H, br s); ESIMS found for C2J⁻120F2N60 m/z 411.0 (M+1).

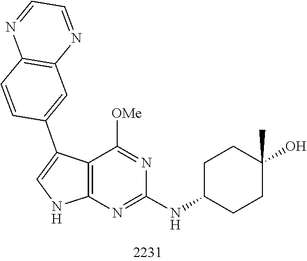 [SMILES: COc1nc(N[C@H]2CC[C@@](C)(O)CC2)nc2[nH]cc(-c3ccc4nccnc4c3)c12]

cis-4-44-Methoxy-5-(quinoxalin-6-yl)-7H-pyrrolo[2,3-d]pyrimidin-2-yl)amino)-1-methylcyclohexan-1-ol 2231.

Brown solid (32 mg, 0.079 mmol, 50.8% yield). ¹H NMR (499 MHz, DMSO-d₆) δppm 1.13 (3 H, s), 1.36 (2 H, td, J=12.87, 4.38 Hz), 1.59 (2 H, br d, J=12.05 Hz), 1.62-1.74 (4 H, m), 3.62-3.73 (1 H, m), 3.97 (3 H, s), 3.99 (1 H, s), 6.48 (1 H, d, J=7.67 Hz), 7.42 (1 H, d, J=2.19 Hz), 8.02 (1 H, d, J=8.76 Hz), 8.19 (1 H, dd, J=8.76, 2.19 Hz), 8.41 (1 H, d, J=2.19 Hz), 8.84 (1 H, d, J=1.64 Hz), 8.89 (1 H, d, J=1.64 Hz), 11.61 (1 H, br s); ESIMS found for C₂₂H₂₄N₆O₂ m/z 405.2 (M+1).

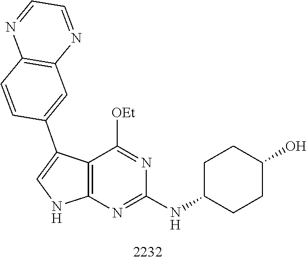 [SMILES: CCOc1nc(N[C@H]2CC[C@@H](O)CC2)nc2[nH]cc(-c3ccc4nccnc4c3)c12]

cis-4-44-Ethoxy-5-(quinoxalin-6-yl)-7H-pyrrolo[2,3-d]pyrimidin-2-yl)amino)cyclohexan-1-ol 2232.

White solid (20.42 mg, 0.051 mmol). ¹H NMR (400 MHz, DMSO-d₆) δppm 1.38 (3 H, br t, J=6.85 Hz), 1.51 (2 H, br d, J=11.49 Hz), 1.60-1.81 (6 H, m), 3.74 (2 H, br s), 4.32 (1 H, br s), 4.47 (2 H, q, J=6.93 Hz), 6.43 (1 H, br d, J=7.46 Hz), 7.43 (1 H, s), 8.01 (1 H, d, J=8.80 Hz), 8.21 (1 H, br d, J=8.80 Hz), 8.50 (1 H, s), 8.83 (1 H, s), 8.89 (1 H, s), 11.59 (1 H, br s); ESIMS found for C22H₂₄N602 m/z 405.0 (M+1).

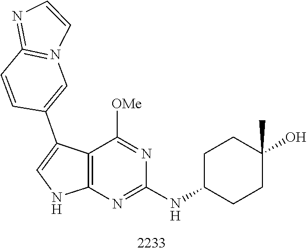

cis-4-45-(Imidazo[1,2-a]pyridin-6-yl)-4-methoxy-7H-pyrrolo[2,3-d]pyrimidin-2-yl)amino)-1-methylcyclohexan-1-ol 2233.

Beige solid (17 mg, 0.043 mmol, 50.4% yield). ¹H NMR (499 MHz, DMSO-d₆) δppm 1.12 (3 H, s), 1.35 (2 H, td, J=12.87, 4.38 Hz), 1.58 (2 H, br d, J=12.32 Hz), 1.62-1.72 (4 H, m), 3.60-3.70 (1 H, m), 3.95 (3 H, s), 3.98 (1 H, s), 6.41 (1 H, d, J=7.94 Hz), 7.16 (1 H, d, J=2.19 Hz), 7.51 (2 H, s), 7.53 (1 H, s), 7.95 (1 H, s), 8.77 (1 H, s), 11.42 (1 H, br s); ESIMS found for C211124N602 m/z 393.2 (M+1).

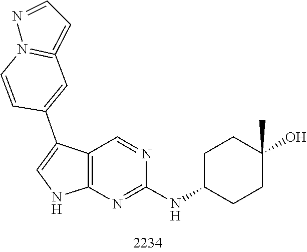

cis-1-Methyl-44(5-(pyrazolo [1,5-a]pyridin-5-yl)-7H-pyrrolo [2,3-d]pyrimidin-2-yl)amino)cyclohexan-1-ol 2234.

Beige solid (16 mg, 0.044 mmol, 38.5% yield). ¹H NMR (499 MHz, DMSO-d₆) δppm 1.13 (3 H, s), 1.36 (2 H, td, J=12.87, 4.93 Hz), 1.59 (2 H, br d, J=12.05 Hz), 1.62-1.72 (4 H, m), 3.59-3.73 (1 H, m), 3.99 (1 H, s), 6.55 (1 H, d, J=1.64 Hz), 6.63 (1 H, br d, J=4.38 Hz), 7.24 (1 H, dd, J=7.39, 1.92 Hz), 7.65 (1 H, s), 7.94 (1 H, d, J=2.19 Hz), 7.96 (1 H, d, J=1.10 Hz), 8.61 (1 H, d, J=7.12 Hz), 9.01 (1 H, s), 11.58 (1 H, br s); ESIMS found for C₂₀I-1₂₂N₆O m/z 363.2 (M +1).

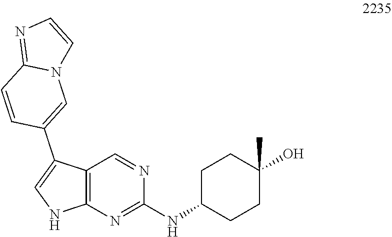 [SMILES: C[C@]1(O)CC[C@@H](Nc2ncc3c(-c4ccc5nccn5c4)c[nH]c3n2)CC1]

cis-4-45-(Imidazo [1,2-a]pyridin-6-yl)-7H-pyrrolo [2,3-d]pyrimidin-2-yl)amino)-1-methylcyclohexan-1-ol 2235.

Beige solid (17 mg, 0.047 mmol, 63.7% yield). ¹H NMR (499 MHz, DMSO-d₆) δppm 1.13 (3 H, s), 1.36 (2 H, td, J=12.94, 4.52 Hz), 1.55-1.61 (2 H, m), 1.62-1.74 (4 H, m), 3.59-3.72 (1 H, m), 4.00 (1 H, s), 6.57-6.66 (1 H, m), 7.51 (1 H, d, J=1.64 Hz), 7.54 (1 H, d, J=1.10 Hz), 7.55-7.59 (1 H, m), 7.59-7.63 (1 H, m), 7.96 (1 H, s), 8.91 (1 H, s), 8.99 (1 H, s), 11.62 (1 H, br s); ESIMS found for C₂₀I-1₂₂N₆O m/z 363.2 (M+1).

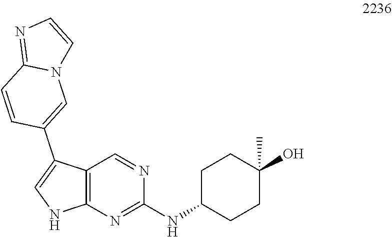 [SMILES: C[C@]1(O)CC[C@H](Nc2ncc3c(-c4ccc5nccn5c4)c[nH]c3n2)CC1]

trans-4-45-(Imidazo[1,2-a]pyridin-6-yl)-7H-pyrrolo[2,3-d]pyrimidin-2-yl)amino)-1-methylcyclohexan-1-ol 2236.

White solid (8 mg, 0.022 mmol, 41.1% yield). ¹H NMR (499 MHz, DMSO-d₆) δppm 1.15 (3 H, s), 1.38-1.51 (4 H, m), 1.61 (2 H, br dd, J=8.49, 5.20 Hz), 1.81-1.92 (2 H, m), 3.73-3.87 (1 H, m), 4.23 (1 H, s), 6.63 (1 H, br d, J=7.39 Hz), 7.52 (1 H, d, J=2.19 Hz), 7.54 (1 H, d, J=0.82 Hz), 7.55-7.58 (1 H, m), 7.59-7.63 (1 H, m), 7.96 (1 H, s), 8.91 (1 H, s), 9.00 (1 H, s), 11.51 (1 H, br s); ESIMS found for C24122N60 m/z 363.2 (M+1).

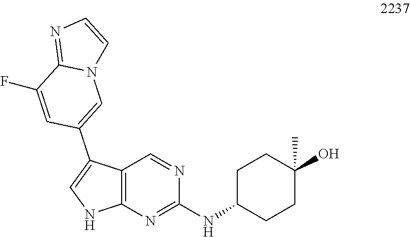 [SMILES: C[C@]1(O)CC[C@H](Nc2ncc3c(-c4cc(F)c5nccn5c4)c[nH]c3n2)CC1]

trans-4-((5-(8-Fluoroimidazo [1,2-a]pyridin-6-yl)-7H-pyrrolo [2,3-d]pyrimidin-2-yl)amino)-1-methylcyclohexan-1-ol 2237.

White solid (2 mg, 0.005 mmol, 9.1% yield). ¹H NMR (499 MHz, DMSO-d₆) δppm 1.15 (3 H, s), 1.37-1.51 (4 H, m), 1.56-1.66 (2 H, m), 1.81-1.89(2 H, m), 3.79(1 H, br s), 4.23 (1 H, br s), 6.66 (1 H, br d, J=7.39 Hz), 7.56-7.62 (3 H, m), 8.09 (1 H, d, J=2.46 Hz), 8.83 (1 H, s), 9.03 (1 H, s), 11.89 (1 H, br s); ESIMS found for C20H_{2,}FN6O m/z 381.2 (M+1).

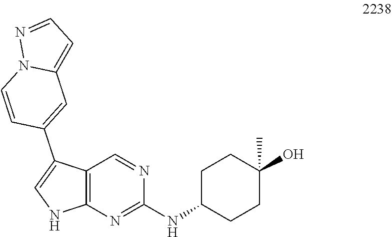 [SMILES: C[C@]1(O)CC[C@H](Nc2ncc3c(-c4ccn5nccc5c4)c[nH]c3n2)CC1]

trans-I-Methyl-44(5-(pyrazolo [1,5-a] pyridin-5-yl)-7H-pyrrolo [2,3-d]pyrimidin-2-yl)amino)cyclohexan-1-ol 2238.

White solid (7 mg, 0.019 mmol, 36.0% yield). ¹H NMR (499 MHz, DMSO-d₆) δppm 1.15 (3 H, s), 1.37-1.50 (4 H, m), 1.55-1.67 (2 H, m), 1.80-1.91 (2 H, m), 3.80(1 H, br s), 4.23 (1 H, s), 6.55 (1 H, s), 6.62-6.72 (1 H, m), 7.24 (1 H, br d, J=7.39 Hz), 7.67 (1 H, d, J=1.64 Hz), 7.94 (1 H, d, J=1.64 Hz), 7.97 (1 H, s), 8.61 (1 H, d, J=7.12 Hz), 9.03 (1 H, s), 11.65 (1 H, br s); ESIMS found for C₂₀1⁻1₂₂N₆O m/z 363.2 (M+1).

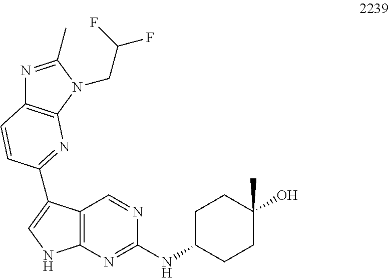 [SMILES: Cc1nc2ccc(-c3c[nH]c4nc(N[C@H]5CC[C@@](C)(O)CC5)ncc34)nc2n1CC(F)F]

cis-4-((5-(3-(2,2-Difluoroethyl)-2-methyl-3H-imidazo [4,5-b]pyridin-5-yl)-7H-pyrrolo [2,3-d]pyrimidin-2-yl)amino)-1-methylcyclohexan-1-ol 2239.

Off-white solid (27 mg, 0.061 mmol, 35.6% yield). ¹H NMR (499 MHz, DMSO-d₆) δppm 1.13 (3 H, s), 1.36 (2 H, td, J=12.87, 4.65 Hz), 1.58 (2 H, br d, J=11.77 Hz), 1.62-1.73 (4 H, m), 2.59 (3 H, s), 3.62-3.75 (1 H, m), 4.01 (1 H, s), 4.82 (2 H, td, J=15.88, 2.74 Hz), 6.55 (2 H, tt, J=54.60, 3.00 Hz), 6.47 (1 H, br d, J=7.67 Hz), 7.71 (1 H, d, J=8.49 Hz), 7.77 (1 H, d, J=2.19 Hz), 7.89 (1 H, d, J=8.21 Hz), 9.36 (1 H, s), 11.49 (1 H, br s); ESIMS found for

C₂₂H₂₅F₂N₇O m/z 442.2 (M+1).

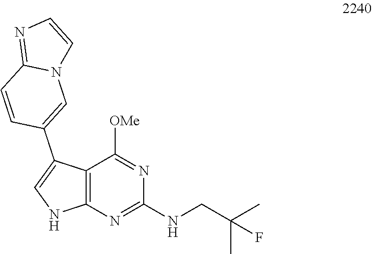 [SMILES: COc1nc(NCC(C)(C)F)nc2[nH]cc(-c3ccc4nccn4c3)c12]

N-(2-Fluoro-2-methylpropyl)-5-(imidazo [1,2-a] pyridin-6-yl)-4-methoxy-7H-pyrrolo [2,3-d] pyrimidin-2-amine 2240.

White solid (10 mg, 0.028 mmol, 21.0% yield). ¹H NMR (499 MHz, DMSO-d₆) δppm 1.35 (6 H, d, J=21.65 Hz), 3.57 (2 H, dd, J=18.75, 6.43 Hz), 3.97 (3 H, s), 6.74 (1 H, t, J=6.43 Hz), 7.20 (1 H, d, J=2.19 Hz), 7.51 (1 H, d, J=1.37 Hz), 7.52 (1 H, s), 7.54 (1 H, s), 7.96 (1 H, s), 8.78 (1 H, s), 11.45 (1 H, br s); ESIMS found for C₁₈F1₁₉FN₆O m/z 355.2 (M+1).

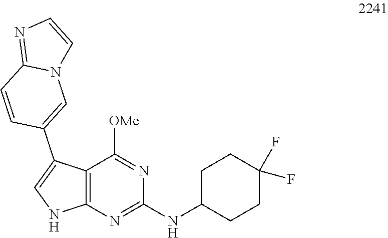 [SMILES: COc1nc(NC2CCC(F)(F)CC2)nc2[nH]cc(-c3ccc4nccn4c3)c12]

N-(4,4-Difluorocyclohexyl)-5-(imidazo[1,2-a]pyridin-6-yl)-4-methoxy-7H-pyrrolo [2,3-d] pyrimidin-2-amine 2241.

Off-white solid (29 mg, 0.073 mmol, 70.0% yield). ¹H NMR (499 MHz, DMSO-d₆) δppm 1.57-1.69(2 H, m), 1.84-2.02 (4 H, m), 2.03-2.16 (2 H, m), 3.86-3.95 (1 H, m), 3.96 (3 H, s), 6.71 (1 H, d, J=7.67 Hz), 7.19 (1 H, d, J=2.46 Hz), 7.51 (1 H, d, J=1.64 Hz), 7.52 (1 H, s), 7.54 (1 H, d, J=1.09 Hz), 7.96 (1 H, s), 8.77 (1 H, s), 11.47 (1 H, br s); ESIMS found for C20H₂₀F2N60 m/z 399.2 (M+1).

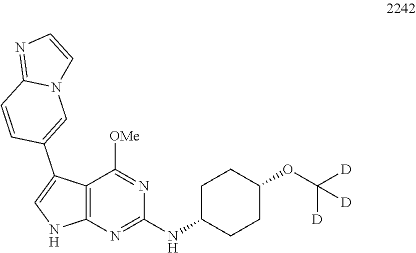 [SMILES: [2H]C([2H])([2H])O[C@H]1CC[C@@H](Nc2nc(OC)c3c(-c4ccc5nccn5c4)c[nH]c3n2)CC1]

5-(Imidazo [1,2-a]pyridin-6-yl)-4-methoxy-N-(cis-4-(methoxy-d₃) cyclohexyl)-7H-pyrrolo [2,3-d] pyrimidin-2-amine 2242.

White solid (54 mg, 0.137 mmol, 53.6% yield). ¹H NMR (499 MHz, DMSO-d₆) δppm 1.44-1.53 (2 H, m), 1.55-1.64 (2 H, m), 1.65-1.73 (2 H, m), 1.81-1.90 (2 H, m), 3.33 -3.37 (1 H, m), 3.73-3.84 (1 H, m), 3.95 (3 H, s), 6.49 (1 H, d, J=7.67 Hz), 7.17 (1 H, d, J=2.19 Hz), 7.51 (1 H, d, J=1.37 Hz), 7.51 (1 H, s), 7.53 (1 H, d, J=0.82 Hz), 7.95 (1 H, s), 8.77 (1 H, s), 11.43 (1 H, br s); ESIMS found for C₂₁1-1₂₁[2H₃]1\1₆0₂ m/z 396.2 (M+1).

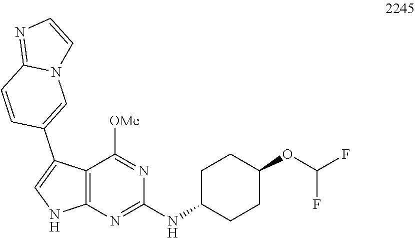 [SMILES: COc1nc(N[C@H]2CC[C@H](OC(F)F)CC2)nc2[nH]cc(-c3ccc4nccn4c3)c12]

N-(trans-4-(Difluoromethoxy)cyclohexyl)-5-(imidazo [1,2-a]pyridin-6-yl)-4-methoxy-7H-pyrrolo [2,3-d]pyrimidin-2-amine 2245.

Off-white solid (20 mg, 0.047 mmol, 27.2% yield). ¹1-1 NMR (499 MHz, DMSO-d₆) δppm 1.33-1.43 (2 H, m), 1.43-1.53 (2 H, m), 2.01 (4 H, br dd, J=10.13, 2.19 Hz), 3.69-3.80 (1 H, m), 3.95 (3 H, s), 4.00-4.10 (1 H, m), 6.54 (1 H, d, J=7.94 Hz), 6.72 (1 H, t, J=76.75 Hz), 7.17 (1 H, d, J=2.46 Hz), 7.50 (1 H, d, J=1.64 Hz), 7.51 (1 H, s), 7.54(1 H, d, J=1.09 Hz), 7.95 (1 H, s), 8.77 (1 H, s), 11.48 (1 H, br s); ESIMS found for C₂₁1-1₂₂F₂N₆O₂m/z 429.2 (M+1).

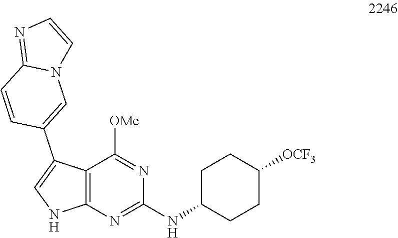 [SMILES: COc1nc(N[C@H]2CC[C@@H](OC(F)(F)F)CC2)nc2[nH]cc(-c3ccc4nccn4c3)c12]

5-(Imidazo [1,2-a]pyridin-6-yl)-4-methoxy-N-(cis-4-(trifluoromethoxy) cyclohexyl)-7H-pyrrolo [2,3-d] pyrimidin-2-amine 2246.

White solid (34 mg, 0.076 mmol, 47.7% yield). ¹H NMR (499 MHz, DMSO-d₆) δppm 1.61-1.70 (2 H, m), 1.70-1.78 (2 H, m), 1.78-1.86 (2 H, m), 1.92-1.98 (2 H, m), 3.80-3.89 (1 H, m), 3.96 (3 H, s), 4.57-4.64 (1 H, m), 6.66 (1 H, d, J=7.67 Hz), 7.18 (1 H, d, J=2.19 Hz), 7.51 (1 H, d, J=1.64 Hz), 7.51 (1 H, s), 7.54 (1 H, d, J=1.10 Hz), 7.96 (1 H, s), 8.77 (1 H, d, J=1.37 Hz), 11.44 (1 H, br s); ESIMS found for C₂₁1-1₂₁F₃N₆O₂ m/z 447.2 (M+1).

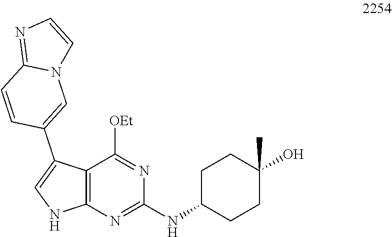 [SMILES: CCOc1nc(N[C@H]2CC[C@@](C)(O)CC2)nc2[nH]cc(-c3ccc4nccn4c3)c12]

cis-4-44-Ethoxy-5-(imidazo [1,2-a]pyridin-6-yl)-7H-pyrrolo [2,3-d]pyrimidin-2-yl)amino)-1-methylcyclohexan-1-ol 2254.

White solid (45.9 mg, 0.113 mmol, 73.9% yield). ¹H NMR (499 MHz, DMSO-d₆) δppm 1.12 (3 H, s), 1.31-1.39 (2 H, m), 1.36 (3 H, t, J=6.98 Hz), 1.55-1.61 (2 H, m), 1.62-1.72 (4 H, m), 3.59-3.73 (1 H, m), 3.99 (1 H, s), 4.42 (2 H, q, J=6.94 Hz), 6.37 (1 H, d, J=7.94 Hz), 7.17 (1 H, d, J=2.19 Hz), 7.51-7.53 (2 H, m), 7.53 (1 H, s), 7.90 (1 H, s), 8.82 (1 H, s), 11.40 (1 H, br s); ESIMS found for C₂₂H₂₆N₆O₂ m/z 407.2 (M+1).

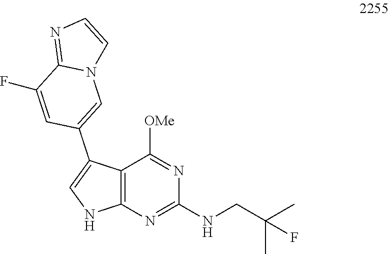 [SMILES: COc1nc(NCC(C)(C)F)nc2[nH]cc(-c3cc(F)c4nccn4c3)c12]

N-(2-Fluoro-2-methylpropyl)-5-(8-fluoroimidazo [1,2-a] pyridin-6-yl)-4-methoxy-7H-pyrrolo [2,3-d]pyrimidin-2-amine 2255.

Off-white solid (7.7 mg, 0.021 mmol, 86.6% yield). ¹1-1 NMR (499 MHz, DMSO-d₆) δppm 1.35 (6 H, d, J=21.40 Hz), 3.57 (2 H, br dd, J=18.62, 6.30 Hz), 4.00 (3 H, s), 6.80 (1 H, br t, J=6.16 Hz), 7.32 (1 H, d, J=2.19 Hz), 7.56 (1 H, br d, J=12.87 Hz), 7.68 (1 H, s), 8.17 (1 H, d, J=1.92 Hz), 8.77 (1 H, s), 11.54 (1 H, br s); ESIMS found for CisH₁₈F₂N₆O m/z 373.15 (M+1).

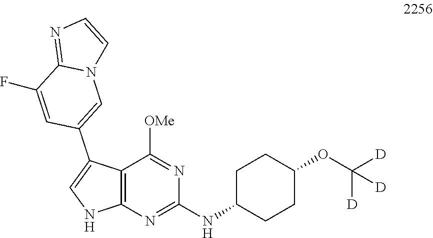 [SMILES: [2H]C([2H])([2H])O[C@H]1CC[C@@H](Nc2nc(OC)c3c(-c4cc(F)c5nccn5c4)c[nH]c3n2)CC1]

5-(8-Fluoroimidazo[1,2-a]pyridin-6-yl)-4-methoxy-N-(cis-4-(methoxy-d₃)cyclohexyl)-7H-pyrrolo [2,3-d] pyrimidin-2-amine 2256.

White solid (6 mg, 0.015 mmol, 39.5% yield). ¹1-1 NMR (499 MHz, DMSO-d₆) δppm 1.44-1.52 (2 H, m), 1.55-1.64 (2 H, m), 1.64-1.71 (2 H, m), 1.80-1.90 (2 H, m), 3.29-3.36 (1 H, m), 3.72-3.83 (1 H, m), 3.97 (3 H, s), 6.53 (1 H, d, J=7.67 Hz), 7.26 (1 H, d, J=2.46 Hz), 7.45 (1 H, dd, J=12.87, 1.10 Hz), 7.59 (1 H, d, J=1.09 Hz), 8.11 (1 H, dd, J=3.15, 0.96 Hz), 8.72 (1 H, d, J=1.10 Hz), 11.49 (1 H, br s); ESIMS found for C₂₁1-1₂₀[2H₃]FN₆O₂ m/z 414.2 (M+1).

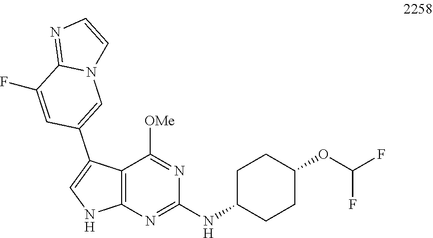 [SMILES: COc1nc(N[C@H]2CC[C@@H](OC(F)F)CC2)nc2[nH]cc(-c3cc(F)c4nccn4c3)c12]

N-(cis-4-(Difluoromethoxy)cyclohexyl)-5-(8-fluoroimidazo [1,2-a]pyridin-6-yl)-4-methoxy-7H-pyrrolo [2,3-d] pyrimidin-2-amine 2258.

Brown solid (11 mg, 0.025 mmol, 24.7% yield). ¹H NMR (500 MHz, DMSO-d₆) δppm 1.61-1.70(4 H, m), 1.73-1.81 (2 H, m), 1.83-1.91 (2 H, m), 3.81 (1 H, br s), 3.98 (3 H, s), 4.29 (1 H, br s), 6.72 (1 H, t, J=76.85 Hz), 6.65 (1 H, d, J=7.68 Hz), 7.26 (1 H, d, J=2.20 Hz), 7.45 (1 H, d, J=12.90 Hz), 7.59 (1 H, s), 8.11 (1 H, d, J=2.47 Hz), 8.72 (1 H, d, J=1.10 Hz), 11.49 (1 H, br s); ESIMS found for C₂₁1-1₂₁F₃N₆O₂ m/z 447.2 (M+1).

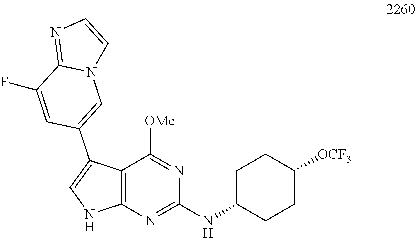 [SMILES: COc1nc(N[C@H]2CC[C@@H](OC(F)(F)F)CC2)nc2[nH]cc(-c3cc(F)c4nccn4c3)c12]

5-(8-Fluoroimidazo [1,2-a]pyridin-6-yl)-4-methoxy-N-(cis-4-(trifluoromethoxy)cyclohexyl)-7H-pyrrolo [2,3-d] pyrimidin-2-amine 2260.

White solid (56 mg, 0.121 mmol, 89.6% yield). ¹H NMR (499 MHz, DMSO-d₆) δppm 1.43-1.52 (2 H, m), 1.54-1.63 (2 H, m), 1.64-1.73 (2 H, m), 1.81-1.90 (2 H, m), 3.35 (1 H, br s), 3.74-3.83 (1 H, m), 3.98 (3 H, s), 6.53 (1 H, d, J=7.94 Hz), 7.26 (1 H, d, J=2.46 Hz), 7.45 (1 H, dd, J=12.87, 1.10 Hz), 7.59 (1 H, d, J=0.82 Hz), 8.11 (1 H, d, J=1.92 Hz), 8.72 (1 H, d, J=1.10 Hz), 11.49 (1 H, br s); ESIMS found for C_{2I}I⁻1₂ₒF₄N₆O₂m/z 465.2 (M+1).

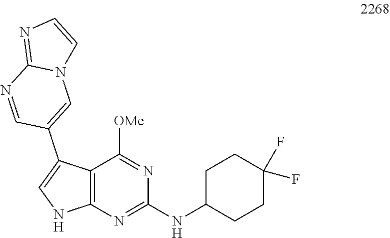 [SMILES: COc1nc(NC2CCC(F)(F)CC2)nc2[nH]cc(-c3cnc4nccn4c3)c12]

N-(4,4-Difluorocyclohexyl)-5-(imidazo[1,2-a]pyrimidin-6-yl)-4-methoxy-7H-pyrrolo [2,3-d]pyrimidin-2-amine 2268.

Beige solid (7 mg, 0.018 mmol, 38.7% yield). ¹H NMR (499 MHz, DMSO-d₆) δppm 1.57-1.71 (2 H, m), 1.85-2.01 (4 H, m), 2.03-2.17 (2 H, m), 3.93 (1 H, br d, J=8.76 Hz), 3.97 (3 H, s), 6.77 (1 H, d, J=7.67 Hz), 7.33 (1 H, s), 7.69 (1 H, d, J=1.10 Hz), 7.95(1 H, d, J=1.37 Hz), 8.81 (1 H, d, J=2.46 Hz), 9.12 (1 H, d, J=2.46 Hz), 11.62 (1 H, br s); ESIMS found for C₁₉H₁₉F₂N₇O m/z 400.2 (M+1).

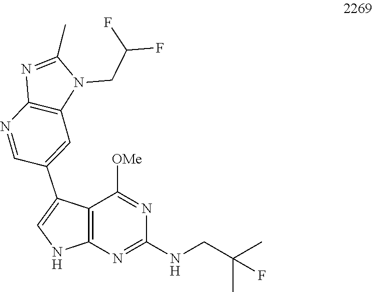 [SMILES: COc1nc(NCC(C)(C)F)nc2[nH]cc(-c3cnc4nc(C)n(CC(F)F)c4c3)c12]

5-(1-(2,2-Difluoroethyl)-2-methyl-1H-imidazo[4,5-b]pyridin-6-yl)-N-(2-fluoro-2-methylpropyl)-4-methoxy-7H-pyrrolo [2,3-d] pyrimidin-2-amine 2269.

Light brown solid (22 mg, 0.051 mmol, 49.7% yield). ¹1-1 NMR (499 MHz, DMSO-d₆) δppm 1.35 (6 H, d, J=21.40 Hz), 2.61 (3 H, s), 3.58 (2 H, dd, J=18.62, 6.30 Hz), 3.93 (3 H, s), 4.74-4.86 (2 H, m), 6.51 (1 H, tt, J=54.30, 2.90 Hz), 6.72 (1 H, br t, J=6.30 Hz), 7.16 (1 H, d, J=2.19 Hz), 8.16 (1 H, d, J=1.92 Hz), 8.59 (1 H, d, J=1.92 Hz), 11.46 (1 H, br s); ESIMS found for C201⁻122F3N70 m/z 434.2 (M+1).

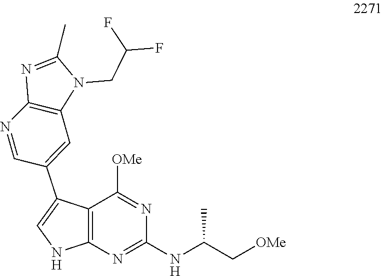 [SMILES: COC[C@@H](C)Nc1nc(OC)c2c(-c3cnc4nc(C)n(CC(F)F)c4c3)c[nH]c2n1]

(R)-5-(1-(2,2-Difluoroethyl)-2-methyl-1H-imidazo[4,5-b]pyridin-6-yl)-4-methoxy-N-(1-methoxypropan-2-yl)-7H-pyrrolo[2,3-d]pyrimidin-2-amine 2271.

White solid (17.4 mg, 0.040 mmol, 27.9% yield). ¹HNMR (499 MHz, DMSO-d₆) δppm 1.16 (3 H, d, J=6.57 Hz), 2.61 (3 H, s), 3.24-3.27 (1 H, m), 3.28 (3 H, s), 3.46 (1 H, dd, J=9.17, 5.61 Hz), 3.91 (3 H, s), 4.18 (1 H, dquin, J=13.55, 6.60, 6.60, 6.60, 6.60 Hz), 4.73-4.87 (2 H, m), 6.38 (1 H, d, J=8.21 Hz), 6.51 (1 H, tt, J=54.60, 2.75 Hz), 7.14 (1 H, d, J=2.19 Hz), 8.14 (1 H, d, J=1.64 Hz), 8.58 (1 H, d, J=2.19 Hz), 11.46 (1 H, br s); ESIMS found for C₂₀H₂₃F₂N₇O₂ m/z 432.2 (M+1).

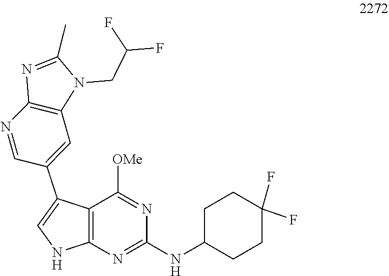 [SMILES: COc1nc(NC2CCC(F)(F)CC2)nc2[nH]cc(-c3cnc4nc(C)n(CC(F)F)c4c3)c12]

N-(4,4-Difluorocyclohexyl)-5-(1-(2,2-difluoroethyl)-2-methyl-1H-imidazo [4,5-b] pyridin-6-yl)-4-methoxy-7H-pyrrolo [2,3-d] pyrimidin-2-amine 2272.

White solid (11.0 mg, 0.023 mmol, 60.1% yield). ¹H NMR (499 MHz, DMSO-d₆) δppm 1.58-1.68 (2 H, m), 1.84-1.95 (2 H, m), 1.96-2.01 (2 H, m), 2.04-2.14 (2 H, m), 2.61 (3 H, s), 3.88-3.98 (1 H, m), 3.92 (3 H, s), 4.73-4.87 (2 H, m), 6.51 (1 H, tt, J=54.35, 3.00 Hz), 6.69 (1 H, d, J=7.94 Hz), 7.14 (1 H, d, J=2.19 Hz), 8.14 (1 H, d, J=1.64 Hz), 8.58 (1 H, d, J=1.92 Hz), 11.48 (1 H, br s); ESIMS found for C₂₂H₂₃F₄N₇O m/z 478.2 (M+1).

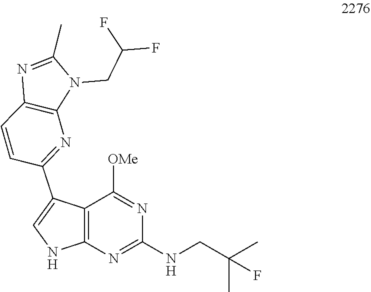 [SMILES: COc1nc(NCC(C)(C)F)nc2[nH]cc(-c3ccc4nc(C)n(CC(F)F)c4n3)c12]

5-(3-(2,2-Difluoroethyl)-2-methyl-3H-imidazo [4,5-b]pyridin-5-yl)-N-(2-fluoro-2-methylpropyl)-4-methoxy-7H-pyrrolo [2,3-d] pyrimidin-2-amine 2276.

White solid (21 mg, 0.049 mmol, 71.2% yield). ¹H NMR (499 MHz, DMSO-d₆) δppm 1.35 (6 H, d, J=21.40 Hz), 2.59 (3 H, s), 3.58 (2 H, dd, J=18.62, 6.30 Hz), 3.98 (3 H, s), 4.75 (2 H, td, J=15.33, 3.83 Hz), 6.53 (2 H, tt, J=54.60, 3.00 Hz), 6.73 (1 H, t, J=6.43 Hz), 7.45 (1 H, d, J=2.19 Hz), 7.83-7.86 (1 H, m), 7.88-7.91 (1 H, m), 11.53 (1 H, br s); ESIMS found for C201⁻122F3N70 m/z 434.2 (M+1).

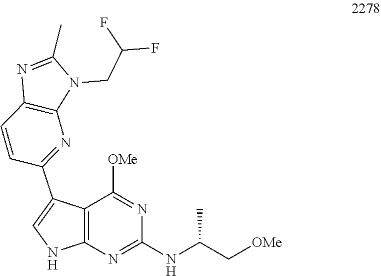 [SMILES: COC[C@@H](C)Nc1nc(OC)c2c(-c3ccc4nc(C)n(CC(F)F)c4n3)c[nH]c2n1]

(R)-5-(3-(2,2-Difluoroethyl)-2-methyl-3H-imidazo [4,5-b]pyridin-5-yl)-4-methoxy-N-(1-methoxypropan-2-yl)-7H-pyrrolo [2,3-d]pyrimidin-2-amine 2278.

White solid (23.9 mg, 0.055 mmol, 30.4% yield). ¹HNMR (499 MHz, DMSO-d₆) δppm 1.17 (3 H, d, J=6.84 Hz), 2.59 (3 H, s), 3.26 (1 H, dd, J=9.17, 6.71 Hz), 3.28 (3 H, s), 3.46 (1 H, dd, J=9.17, 5.61 Hz), 3.97 (3 H, s), 4.18 (1 H, dt, J=13.76, 6.67 Hz), 4.75 (2 H, td, J=15.33, 3.56 Hz), 6.39 (1 H, d, J=8.21 Hz), 6.53 (1 H, tt, J=55.10, 3.55 Hz), 7.44 (1 H, d, J=2.46 Hz), 7.83-7.87 (1 H, m), 7.88-7.91 (1 H, m), 11.54 (1 H, br s); ESIMS found for C₂oH₂₃F₂N₇O₂ m/z 432.2 (M+1).

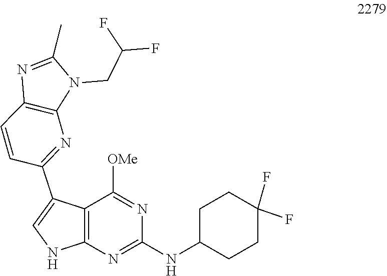 [SMILES: COc1nc(NC2CCC(F)(F)CC2)nc2[nH]cc(-c3ccc4nc(C)n(CC(F)F)c4n3)c12]

N-(4,4-Difluorocyclohexyl)-5-(3-(2,2-difluoroethyl)-2-methyl-3H-imidazo [4,5-b] pyridin-5-yl)-4-methoxy-7H-pyrrolo [2,3-d] pyrimidin-2-amine 2279.

White solid (8.9 mg, 0.019 mmol, 54.8% yield). ¹H NMR (499 MHz, DMSO-d₆) δppm 1.58-1.71 (2 H, m), 1.84-1.95 (2 H, m), 1.96-2.02 (2 H, m), 2.04-2.17 (2 H, m), 2.59 (3 H, s), 3.87-3.96 (1 H, m), 3.98 (3 H, s), 4.74 (2 H, td, J=15.26, 3.42 Hz), 6.53 (1 H, tt, J=54.85, 3.55 Hz), 6.70 (1 H, d, J=7.67 Hz), 7.44 (1 H, d, J=2.46 Hz), 7.83-7.87 (1 H, m), 7.88-7.91 (1 H, m), 11.56 (1 H, br s); ESIMS found for C₂₂H₂₃F₄N₇O m/z 478.2 (M+1).

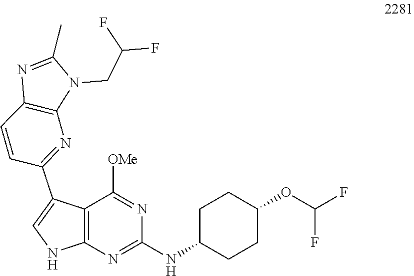 [SMILES: COc1nc(N[C@H]2CC[C@@H](OC(F)F)CC2)nc2[nH]cc(-c3ccc4nc(C)n(CC(F)F)c4n3)c12]

5-(3-(2,2-Difluoroethyl)-2-methyl-3H-imidazo [4,5-b]pyridin-5-yl)-N-(cis-4-(difluoromethoxy)cyclohexyl)-4-methoxy-7H-pyrrolo[2,3-d]pyrimidin-2-amine 2281.

Off-white solid (5 mg, 0.010 mmol, 10.1% yield). ¹1-1 NMR (499 MHz, DMSO-d₆) δppm 1.62-1.72 (4 H, m), 1.75-1.81 (2 H, m), 1.84-1.90 (2 H, m), 3.77-3.85 (1 H, m), 3.97 (3 H, s), 4.29 (1 H, br s), 4.74 (2 H, td, J=15.47, 3.83 Hz), 6.53 (1 H, tt, J=55.15, 3.55 Hz), 6.73 (1 H, t, J=77.05 Hz), 6.60 (1 H, d, J=7.67 Hz), 7.43 (1 H, d, J=2.46 Hz), 7.83-7.87 (1 H, m), 7.87-7.91 (1 H, m); ESIMS found for C23H₂₅F4N702 m/z 508.2 (M+1).

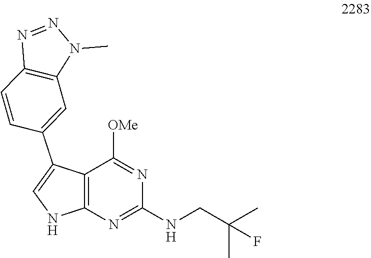 [SMILES: COc1nc(NCC(C)(C)F)nc2[nH]cc(-c3ccc4nnn(C)c4c3)c12]

N-(2-Fluoro-2-methylpropyl)-4-methoxy-5-(1-methyl-1H-benzo[d][1,2,3] triazol-6-yl)-7H-pyrrolo [2,3-d]pyrimidin-2-amine 2283.

White solid (11 mg, 0.030 mmol, 33.1% yield). ¹H NMR (499 MHz, DMSO-d₆) δppm 1.35 (6 H, d, J=21.40 Hz), 3.58 (2 H, dd, J=18.75, 6.43 Hz), 3.97 (3 H, s), 4.30 (3 H, s), 6.75 (1 H, t, J=6.43 Hz), 7.27 (1 H, d, J=2.46 Hz), 7.69 (1 H, dd, J=8.62, 1.51 Hz), 7.94 (1 H, d, J=8.76 Hz), 8.04 (1 H, s), 11.52 (1 H, br s); ESIMS found for C₁₈1-1₂₀FN₇O m/z 370.2 (M+1).

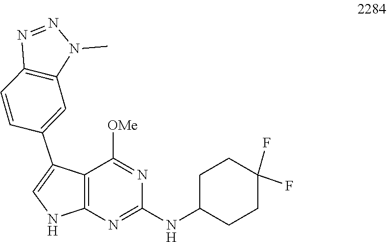 [SMILES: COc1nc(NC2CCC(F)(F)CC2)nc2[nH]cc(-c3ccc4nnn(C)c4c3)c12]

N-(4,4-Difluorocyclohexyl)-4-methoxy-5-(1-methyl-1H-benzo[d][1,2,3] triazol-6-yl)-7H-pyrrolo [2,3-d]pyrimidin-2-amine 2284.

White solid (10 mg, 0.024 mmol, 13.8% yield). ¹H NMR (499 MHz, DMSO-d₆) δppm 1.58-1.70 (2 H, m), 1.85-2.01 (4 H, m), 2.05-2.16 (2 H, m), 3.89-3.96 (1 H, m), 3.96 (3 H, s), 4.30 (3 H, s), 6.71 (1 H, d, J=7.67 Hz), 7.26 (1 H, d, J=2.19 Hz), 7.69 (1 H, dd, J=8.76, 1.37 Hz), 7.94 (1 H, d, J=8.76 Hz), 8.03 (1 H, s), 11.54 (1 H, br s); ESIMS found for C₂₀1-1₂₁F₂N₇O m/z 414.2 (M+1).

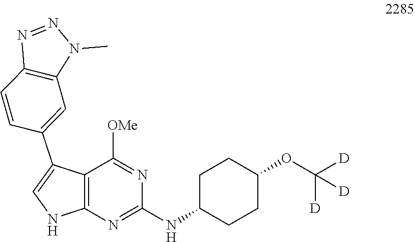 [SMILES: [2H]C([2H])([2H])O[C@H]1CC[C@@H](Nc2nc(OC)c3c(-c4ccc5nnn(C)c5c4)c[nH]c3n2)CC1]

4-Methoxy-N-(cis-4-(methoxy-d₃)cyclohexyl)-5-(1-methyl-1H-benzo[d] [1,2,3]triazol-6-yl)-7H-pyrrolo [2,3-d]pyrimidin-2-amine 2285.

White solid (13 mg, 0.032 mmol, 34.3% yield). ¹H NMR (499 MHz, DMSO-d₆) δppm 1.43-1.52 (2 H, m), 1.53-1.65 (2 H, m), 1.65-1.72 (2 H, m), 1.80-1.90 (2 H, m), 3.34-3.36 (1 H, m), 3.72-3.84 (1 H, m), 3.95 (3 H, s), 4.29 (3 H, s), 6.50 (1 H, d, J=7.94 Hz), 7.23 (1 H, d, J=2.46 Hz), 7.69 (1 H, dd, J=8.76, 1.37 Hz), 7.94 (1 H, d, J=8.76 Hz), 8.03 (1 H, s), 11.50 (1 H, br s); ESIMS found for C₂₁1-1₂₂[2H₃]N₇O₂ m/z 411.2 (M+1).

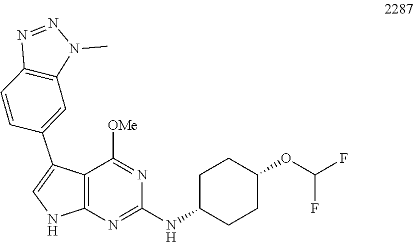 [SMILES: COc1nc(N[C@H]2CC[C@@H](OC(F)F)CC2)nc2[nH]cc(-c3ccc4nnn(C)c4c3)c12]

N-(cis-4-(Difluoromethoxy)cyclohexyl)-4-methoxy-5-(1-methyl-1H-benzo[d] [1,2,3]triazol-6-yl)-7H-pyrrolo [2,3-d]pyrimidin-2-amine 2287.

White solid (31 mg, 0.070 mmol, 65.3% yield). ¹H NMR (499 MHz, DMSO-d₆) δppm 1.59-1.72 (4 H, m), 1.73-1.82 (2 H, m), 1.88 (2 H, br dd, J=9.45, 4.52 Hz), 3.78-3.88 (1 H, m), 3.96 (3 H, s), 4.28-4.31 (1 H, m), 4.29 (3 H, s), 6.73 (1 H, t, J=76.85 Hz), 6.62 (1 H, d, J=7.67 Hz), 7.24 (1 H, d, J=2.46 Hz), 7.69 (1 H, dd, J=8.76, 1.64 Hz), 7.94 (1 H, d, J=8.49 Hz), 8.03 (1 H, s), 11.50 (1 H, br s); ESIMS found for C₂₁1-1₂₃F₂N₇O₂ m/z 444.2 (M+1).

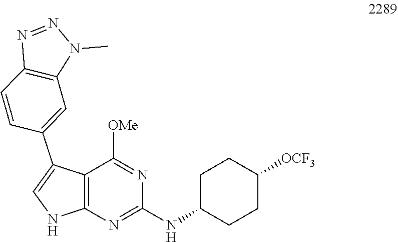 [SMILES: COc1nc(N[C@H]2CC[C@@H](OC(F)(F)F)CC2)nc2[nH]cc(-c3ccc4nnn(C)c4c3)c12]

4-Methoxy-5-(1-methyl-1H-benzo[d][1,2,3]triazol-6-yl)-N-(cis-4-(trifluoromethoxy)cyclohexyl)-7H-pyrrolo[2,3-d]pyrimidin-2-amine 2289.

White solid (4 mg, 0.009 mmol, 5.1% yield). ¹H NMR (499 MHz, DMSO-d₆) δppm 1.61-1.78 (4 H, m), 1.79-1.88 (2 H, m), 1.96 (2 H, br dd, J=8.90, 4.52 Hz), 3.79-3.91 (1 H, m), 3.96 (3 H, s), 4.29 (3 H, s), 4.57-4.64 (1 H, m), 6.66 (1 H, d, J=7.67 Hz), 7.25 (1 H, d, J=2.19 Hz), 7.69 (1 H, dd, J=8.76, 1.37 Hz), 7.94 (1 H, d, J=8.49 Hz), 8.03 (1 H, s); ESIMS found for C211⁻122F3N702 m/z 462.2 (M+1).

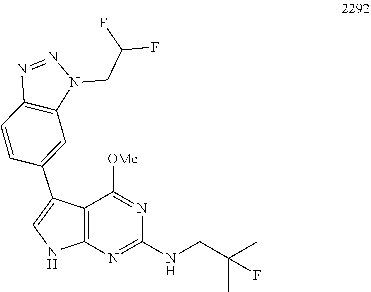 [SMILES: COc1nc(NCC(C)(C)F)nc2[nH]cc(-c3ccc4nnn(CC(F)F)c4c3)c12]

5-(1-(2,2-Difluoroethyl)-1H-benzo[d][1,2,3]triazol-6-yl)-N-(2-fluoro-2-methylpropyl)-4-methoxy-7H-pyrrolo [2,3-d] pyrimidin-2-amine 2292.

White solid (13 mg, 0.031 mmol, 31.2% yield). ¹H NMR (499 MHz, DMSO-d₆) δppm 1.35 (6 H, d, J=21.40 Hz), 3.58 (2 H, dd, J=18.48, 6.43 Hz), 3.95 (3 H, s), 5.28 (2 H, td, J=16.08, 2.87 Hz), 6.61 (1 H, tt, J=54.10, 3.00 Hz), 6.77 (1 H, t, J=6.43 Hz), 7.26 (1 H, d, J=2.46 Hz), 7.71 (1 H, dd, J=8.62, 1.51 Hz), 7.99 (1 H, d, J=8.76 Hz), 8.12 (1 H, s), 11.54 (1 H, br s); ESIMS found for C₁₉H₂₀F₃N₇O m/z 420.2 (M+1).

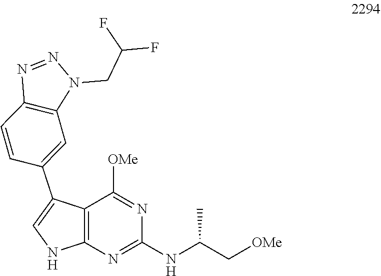 [SMILES: COC[C@@H](C)Nc1nc(OC)c2c(-c3ccc4nnn(CC(F)F)c4c3)c[nH]c2n1]

(R)-5-(1-(2,2-Difluoroethyl)-1H-benzo[d][1,2,3]triazol-6-yl)-4-methoxy-N-(1-methoxypropan-2-yl)-7H-pyrrolo [2,3-d] pyrimidin-2-amine 2294.

White solid (11.1 mg, 0.027 mmol, 71.4% yield). ¹HNMR (499 MHz, DMSO-d₆) δppm 1.16(3 H, d, J=6.57 Hz), 3.24-3.27 (1 H, m), 3.28(3 H, s), 3.46(1 H, dd, J=9.31, 5.48 Hz), 3.93 (3 H, s), 4.18 (1 H, dt, J=13.62, 6.74 Hz), 5.27 (2 H, td, J=15.95, 2.87 Hz), 6.43 (1 H, d, J=8.21 Hz), 6.61 (2 H, tt, J=54.30, 3.00 Hz), 7.24 (1 H, d, J=2.46 Hz), 7.71 (1 H, dd, J=8.76, 1.37 Hz), 7.99 (1 H, d, J=8.76 Hz), 8.11 (1 H, s), 11.54 (1 H, br s); ESIMS found for C₁₈1-1₁₆F₅N₇O m/z 418.2 (M+1).

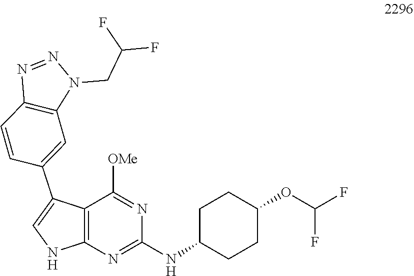 [SMILES: COc1nc(N[C@H]2CC[C@@H](OC(F)F)CC2)nc2[nH]cc(-c3ccc4nnn(CC(F)F)c4c3)c12]

5-(1-(2,2-Difluoroethyl)-1H-benzo[d][1,2,3]triazol-6-yl)-N-(cis-4-(difluoromethoxy)cyclohexyl)-4-methoxy-7H-pyrrolo[2,3-d]pyrimidin-2-amine 2296.

Off-white solid (3 mg, 0.006 mmol, 5.6% yield). ¹H NMR (499 MHz, DMSO-d₆) δppm 1.62-1.71 (4 H, m), 1.75-1.81 (2 H, m), 1.86-1.91 (2 H, m), 3.80-3.87 (1 H, m), 3.94 (3 H, s), 4.29 (1 H, br s), 5.27 (2 H, td, J=16.02, 2.74 Hz), 6.61 (1 H, tt, J=54.30, 3.05 Hz), 6.73 (1 H, t, J=77.05 Hz), 6.63 (1 H, d, J=7.67 Hz), 7.23 (1 H, d, J=2.46 Hz), 7.71 (1 H, dd, J=8.62, 1.51 Hz), 7.99 (1 H, d, J=8.76 Hz), 8.11 (1 H, s), 11.52 (1 H, br s); ESIMS found for C₂₂H₂₃F₄N₇O₂ m/z 494.2 (M+1).

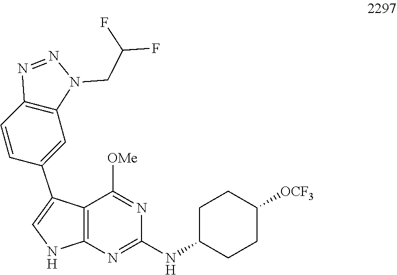 [SMILES: COc1nc(N[C@H]2CC[C@@H](OC(F)(F)F)CC2)nc2[nH]cc(-c3ccc4nnn(CC(F)F)c4c3)c12]

5-(1-(2,2-Difluoroethyl)-1H-benzo[d][1,2,3]triazol-6-yl)-4-methoxy-N-(cis-4-(trifluoromethoxy)cyclohexyl)-7H-pyrrolo [2,3-d] pyrimidin-2-amine 2297.

Off-white solid (9.5 mg, 0.019 mmol, 47.9% yield). ¹H NMR (499 MHz, DMSO-d₆) δppm 1.61-1.77(4 H, m), 1.79-1.86 (2 H, m), 1.92-2.01 (2 H, m), 3.80-3.89(1 H, m), 3.94 (3 H, s), 4.56-4.65 (1 H, m), 5.27 (2 H, td, J=15 .9 5 , 2.60 Hz), 6.60 (1 H, tt, J=54.30, 3.05 Hz), 6.68 (1 H, br d, J=7.67 Hz), 7.24 (1 H, d, J=2.46 Hz), 7.71 (1 H, dd, J=8.76, 1.37 Hz), 7.99 (1 H, d, J=8.76 Hz), 8.11 (1 H, s), 11.53 (1 H, br s); ESIMS found for C₂₂H₂₂F₅N₇O₂m/z 512.2 (M+1).

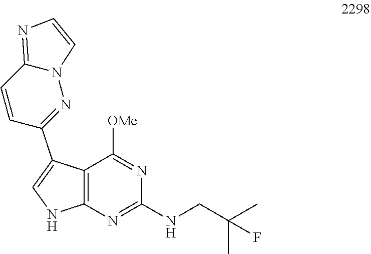 [SMILES: COc1nc(NCC(C)(C)F)nc2[nH]cc(-c3ccc4nccn4n3)c12]

N-(2-Fluoro-2-methylpropyl)-5-(imidazo [1,2-b]pyridazin-6-yl)-4-methoxy-7H-pyrrolo [2,3-d]pyrimidin-2-amine 2298.

White solid (1.5 mg, 0.004 mmol, 2.6% yield). ¹H NMR (499 MHz, DMSO-d₆) δppm 1.35 (6 H, d, J=21.40 Hz), 3.58 (2 H, dd, J=18.62, 6.30 Hz), 3.97 (3 H, s), 6.85 (1 H, br t, J=6.43 Hz), 7.43 (1 H, d, J=2.19 Hz), 7.64 (1 H, d, J=9.58 Hz), 7.72 (1 H, d, J=1.09 Hz), 8.05 (1 H, d, J=9.31 Hz), 8.20 (1 H, s), 11.74 (1 H, br s); ESIMS found for C₁₇HisFN₇O m/z 356.1 (M+1).

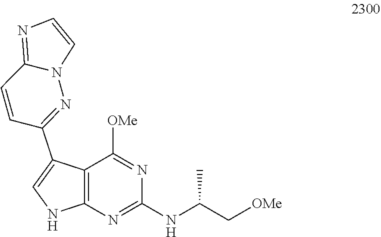 [SMILES: COC[C@@H](C)Nc1nc(OC)c2c(-c3ccc4nccn4n3)c[nH]c2n1]

(R)-5-(Imidazo [1,2-b] pyridazin-6-yl)-4-methoxy-N-(1-methoxypropan-2-yl)-7H-pyrrolo [2,3-d]pyrimidin-2-amine 2300.

White solid (4.9 mg, 0.014 mmol, 14.2% yield). ¹H NMR (499 MHz, DMSO-d₆) δppm 1.16 (3 H, br d, J=6.57 Hz), 3.20-3.27 (1 H, m), 3.28(3 H, s), 3.46(1 H, br dd, J=9.17, 5.61 Hz), 3.95 (3 H, s), 4.18 (1 H, dt, J=13.42, 6.43 Hz), 6.51 (1 H, br d, J=8.21 Hz), 7.41 (1 H, d, J=2.19 Hz), 7.64 (1 H, d, J=9.31 Hz), 7.72 (1 H, s), 8.05 (1 H, d, J=9.31 Hz), 8.20 (1 H, s), 11.74 (1 H, br s); ESIMS found for C₁₇H₁₉N₇O₂ m/z 354.2 (M+1).

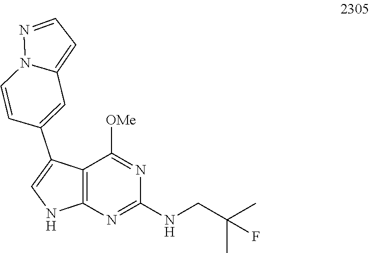 [SMILES: COc1nc(NCC(C)(C)F)nc2[nH]cc(-c3ccn4nccc4c3)c12]

N-(2-Fluoro-2-methylpropyl)-4-methoxy-5-(pyrazolo [1,5-a]pyridin-5-yl)-7H-pyrrolo [2,3-d]pyrimidin-2-amine 2305.

White solid (27 mg, 0.076 mmol, 29.5% yield). ¹H NMR (499 MHz, DMSO-d₆) δppm 1.35 (6 H, d, J=21.40 Hz), 3.57 (2 H, dd, J=18.62, 6.57 Hz), 3.99 (3 H, s), 6.55 (1 H, d, J=1.64 Hz), 6.77 (1 H, t, J=6.43 Hz), 7.19 (1 H, dd, J=7.39, 1.92 Hz), 7.36 (1 H, d, J=1.92 Hz), 7.93 (1 H, d, J=2.19 Hz), 7.99 (1 H, d, J=1.37 Hz), 8.58 (1 H, d, J=7.39 Hz), 11.54 (1 H, br s); ESIMS found for C₁₈H₁₉FN60 m/z 355.2 (M+1).

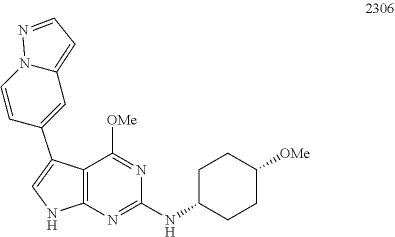 [SMILES: COc1nc(N[C@H]2CC[C@@H](OC)CC2)nc2[nH]cc(-c3ccn4nccc4c3)c12]

4-Methoxy-N-(cis-4-methoxycyclohexyl)-5-(pyrazolo [1,5-a] pyridin-5-yl)-7H-pyrrolo [2,3-d] pyrimidin-2-amine 2306.

Beige solid (103 mg, 0.263 mmol, 70.4% yield). ¹H NMR (499 MHz, DMSO-d₆) δppm 1.44-1.53 (2 H, m), 1.55-1.64 (2 H, m), 1.65-1.72 (2 H, m), 1.81-1.89 (2 H, m), 3.32 (3 H, s), 3.34-3.38 (1 H, m), 3.73-3.85 (1 H, m), 3.97 (3 H, s), 6.52 (1 H, d, J=7.67 Hz), 6.54 (1 H, d, J=1.37 Hz), 7.18 (1 H, dd, J=7.39, 1.92 Hz), 7.32 (1 H, d, J=2.46 Hz), 7.93 (1 H, d, J=2.19 Hz), 7.99 (1 H, d, J=1.10 Hz), 8.57 (1 H, d, J=7.39 Hz), 11.52 (1 H, br s); ESIMS found for

C₂₁11₂₄N₆O₂ m/z 393.2 (M+1).

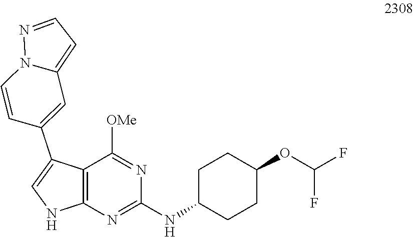 [SMILES: COc1nc(N[C@H]2CC[C@H](OC(F)F)CC2)nc2[nH]cc(-c3ccn4nccc4c3)c12]

N-(trans-4-(Difluoromethoxy)cyclohexyl)-4-methoxy-5-(pyrazolo [1,5-a] pyridin-5-yl)-7H-pyrrolo [2,3-d] pyrimidin-2-amine 2308.

White solid (38 mg, 0.089 mmol, 51.7% yield). ¹H NMR (499 MHz, DMSO-d₆) δppm 1.33-1.43 (2 H, m), 1.43-1.53 (2 H, m), 2.01 (4 H, br d, J=9.58 Hz), 3.68-3.81 (1 H, m), 3.97 (3 H, s), 4.05 (1 H, br t, J=10.27 Hz), 6.54 (1 H, s), 6.57 (1 H, br d, J=7.67 Hz), 6.72 (1 H, t, J=77.05 Hz), 7.18 (1 H, d, J=7.12 Hz), 7.33 (1 H, d, J=1.37 Hz), 7.93 (1 H, d, J=1.92 Hz), 7.98 (1 H, s), 8.57 (1 H, d, J=7.39 Hz), 11.57 (1 H, br s); ESIMS found for C_{2I}F1₂₂F₂N₆O₂m/z 429.2 (M+1).

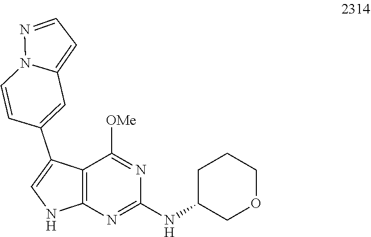 [SMILES: COc1nc(N[C@@H]2CCCOC2)nc2[nH]cc(-c3ccn4nccc4c3)c12]

(R)-4-Methoxy-5-(pyrazolo[1,5-a]pyridin-5-yl)-N-(tetrahydro-2H-pyran-3-yl)-7H-pyrrolo[2,3-d]pyrimidin-2-amine 2314.

White solid (52.0 mg, 0.143 mmol, 54.3% yield). ¹HNMR (499 MHz, DMSO-d₆) δppm 1.51-1.64 (2 H, m), 1.67-1.75 (1 H, m), 1.94-2.02 (1 H, m), 3.11 (1 H, t, J=9.86 Hz), 3.24-3.31 (1 H, m), 3.73-3.79 (1 H, m), 3.85-3.91 (1 H, m), 3.91-3.96 (1 H, m), 3.97 (3 H, s), 6.54 (1 H, d, J=1.64 Hz), 6.56 (1 H, d, J=7.94 Hz), 7.18 (1 H, dd, J=7.39, 1.92 Hz), 7.35 (1 H, d, J=2.46 Hz), 7.93 (1 H, d, J=2.19 Hz), 7.98 (1 H, d, J=1.09 Hz), 8.58 (1 H, d, J=7.39 Hz), 11.57 (1 H, br s); ESIMS found for C₁₉H₂oN₆O₂ m/z 365.15 (M+1).

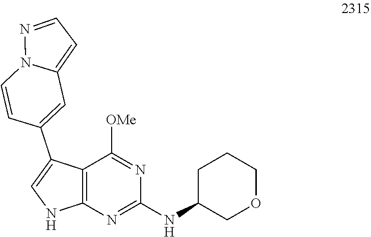 [SMILES: COc1nc(N[C@H]2CCCOC2)nc2[nH]cc(-c3ccn4nccc4c3)c12]

(S)-4-Methoxy-5-(pyrazolo[1,5-a]pyridin-5-yl)-N-(tetrahydro-2H-pyran-3-yl)-7H-pyrrolo[2,3-d]pyrimidin-2-amine 2315.

Beige solid (134 mg, 0.368 mmol, 95.7% yield). ¹H NMR (499 MHz, DMSO-d₆) δppm 1.50-1.65 (2 H, m), 1.67-1.75 (1 H, m), 1.98 (1 H, br d, J=8.21 Hz), 3.11 (1 H, t, J=9.72 Hz), 3.28 (1 H, td, J=10.88, 2.33 Hz), 3.76 (1 H, br d, J=10.95 Hz), 3.85-3.91 (1 H, m), 3.91-3.96 (1 H, m), 3.97 (3 H, s), 6.54 (1 H, d, J=1.64 Hz), 6.56 (1 H, d, J=7.94 Hz), 7.18 (1 H, dd, J=7.39, 1.92 Hz), 7.35 (1 H, d, J=2.46 Hz), 7.93 (1 H, d, J=2.19 Hz), 7.99 (1 H, d, J=1.10 Hz), 8.58 (1 H, d, J=7.39 Hz), 11.57 (1 H, br s); ESIMS found for C₁₉H₂oN₆O₂ m/z 365.2 (M+1).

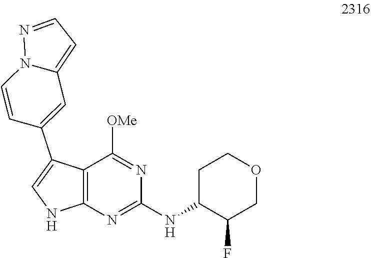

N-((3 S,4R)-3-Fluorotetrahydro-2H-pyran-4-yl)-4-methoxy-5-(pyrazolo [1,5-a] pyridin-5-yl)-7H-pyrrolo [2,3-d] pyrimidin-2-amine 2316.

White solid (4 mg, 0.011 mmol, 15.3% yield). ¹H NMR (499 MHz, DMSO-d₆) δppm 1.54-1.67 (1 H, m), 1.99-2.11 (1 H, m), 3.38-3.43 (1 H, m), 3.43-3.49(1 H, m), 3.80-3.89 (1 H, m), 4.00 (3 H, s), 4.00-4.05 (1 H, m), 4.16-4.28 (1 H, m), 4.52-4.67 (1 H, m), 6.55 (1 H, d, J=1.64 Hz), 6.98 (1 H, d, J=8.21 Hz), 7.18 (1 H, dd, J=7.39, 1.92 Hz), 7.37 (1 H, d, J=2.46 Hz), 7.93 (1 H, d, J=2.19 Hz), 7.99 (1 H, d, J=1.09 Hz), 8.58 (1 H, d, J=7.39 Hz), 11.61 (1 H, br s); ESIMS found for C₁₉H₁₉FN602 m/z 383.2 (M+1).

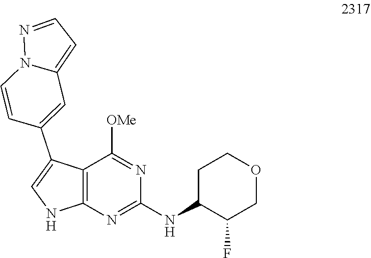

N-((3R,4R)-3-Fluorotetrahydro-2H-pyran-4-yl)-4-methoxy-5-(pyrazolo[1,5-a] pyridin-5-yl)-7H-pyrrolo [2,3-d] pyrimidin-2-amine 2317.

Off-white solid (12 mg, 0.031 mmol, 25.9% yield). ¹H NMR (499 MHz, DMSO-d₆) δppm 1.54-1.68(1 H, m), 2.00-2.11 (1 H, m), 3.38-3.43 (1 H, m), 3.43-3.49 (1 H, m), 3.80-3.89 (1 H, m), 3.98-4.05 (1 H, m), 3.99 (3 H, s), 4.17-4.28 (1 H, m), 4.50-4.69 (1 H, m), 6.55 (1 H, d, J=2.19 Hz), 6.98 (1 H, d, J=7.94 Hz), 7.18 (1 H, dd, J=7.39, 1.92 Hz), 7.37 (1 H, d, J=2.46 Hz), 7.93 (1 H, d, J=2.19 Hz), 7.99 (1 H, d, J=1.10 Hz), 8.58 (1 H, d, J=7.39 Hz), 11.61 (1 H, br s); ESIMS found for C₁₉H₁₉FN₆O₂ m/z 383.15 (M+1).

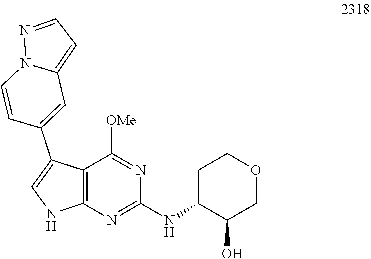 [SMILES: COc1nc(N[C@@H]2CCOC[C@H]2O)nc2[nH]cc(-c3ccn4nccc4c3)c12]

(3 S,4R)-4-((4-Methoxy-5-(pyrazolo [1,5-a]pyridin-5-yl)-7H-pyrrolo [2,3-d]pyrimidin-2-yl)amino)tetrahydro-2H-pyran-3-ol 2318.

Beige solid (22 mg, 0.058 mmol, 36.9% yield). ¹H NMR (499 MHz, DMSO-d₆) δppm 1.43-1.56 (1 H, m), 2.05 (1 H, br d, J=11.77 Hz), 3.05 (1 H, br t, J=10.40 Hz), 3.36 (1 H, br s), 3.47-3.57 (1 H, m), 3.76-3.89 (3 H, m), 3.98 (3 H, s), 5.03 (1 H, br d, J=4.65 Hz), 6.54 (1 H, d, J=1.37 Hz), 6.57 (1 H, br d, J=7.39 Hz), 7.18 (1 H, dd, J=7.26, 1.51 Hz), 7.34 (1 H, d, J=2.19 Hz), 7.93 (1 H, d, J=1.92 Hz), 7.99 (1 H, s), 8.58 (1 H, d, J=7.39 Hz), 11.57 (1 H, br s); ESIMS found for C₁₉H₂oN603 m/z 381.2 (M+1).

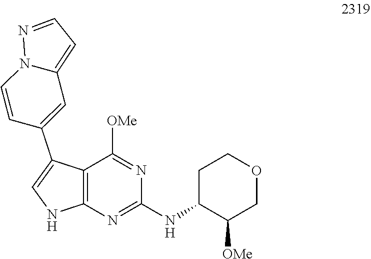 [SMILES: COc1nc(N[C@@H]2CCOC[C@H]2OC)nc2[nH]cc(-c3ccn4nccc4c3)c12]

4-Methoxy-N-((3S,4R)-3-methoxytetrahydro-2H-pyran-4-yl)-5-(pyrazolo [1,5-a] pyridin-5-yl)-7H-pyrrolo [2,3-d] pyrimidin-2-amine 2319.

Beige solid (102 mg, 0.259 mmol, 79.8% yield). ¹H NMR (499 MHz, DMSO-d₆) δppm 1.46-1.58 (1 H, m), 1.95-2.04 (1 H, m), 3.11 (1 H, dd, J=10.81, 9.17 Hz), 3.28-3.31 (1 H, m), 3.33 (3 H, s), 3.35-3.39 (1 H, m), 3.81 (1 H, dt, J=11.43, 3.73 Hz), 3.93-4.01 (1 H, m), 3.99 (3 H, s), 4.03 (1 H, dd, J=11.22, 4.11 Hz), 6.54 (1 H, d, J=1.92 Hz), 6.79 (1 H, d, J=7.94 Hz), 7.18 (1 H, dd, J=7.12, 1.92 Hz), 7.34 (1 H, d, J=2.19 Hz), 7.93 (1 H, d, J=2.19 Hz), 7.99 (1 H, d, J=1.09 Hz), 8.58 (1 H, d, J=7.39 Hz), 11.54 (1 H, br s); ESIMS found for C₂₀H₂₂N₆O₃ m/z 395.2 (M+1).

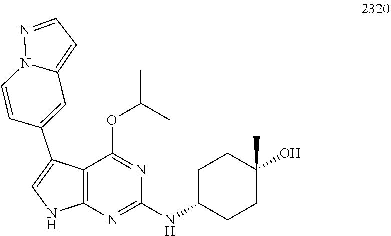 [SMILES: CC(C)Oc1nc(N[C@H]2CC[C@@](C)(O)CC2)nc2[nH]cc(-c3ccn4nccc4c3)c12]

cis-4-44-Isopropoxy-5-(pyrazolo[1,5-a]pyridin-5-yl)-7H-pyrrolo[2,3-d]pyrimidin-2-yl)amino)-1-methylcyclohexan-1-ol 2320.

Off-white solid (24 mg, 0.057 mmol, 26.6% yield). ¹H NMR (499 MHz, DMSO-d₆) δppm 1.12 (3 H, s), 1.31-1.36 (2 H, m), 1.37 (6 H, d, J=6.30 Hz), 1.55-1.61 (2 H, m), 1.62-1.73 (4 H, m), 3.56-3.71 (1 H, m), 3.99 (1 H, s), 5.45 (1 H, dt, J=12.32, 6.16 Hz), 6.37 (1 H, br d, J=7.67 Hz), 6.50 (1 H, d, J=2.19 Hz), 7.20 (1 H, dd, J=7.39, 1.92 Hz), 7.31 (1 H, d, J=2.19 Hz), 7.92 (1 H, d, J=2.19 Hz), 8.08 (1 H, s), 8.57 (1 H, d, J=7.39 Hz), 11.45 (1 H, br s); ESIMS found for C23H₂₈N602 m/z 421.2 (M+1).

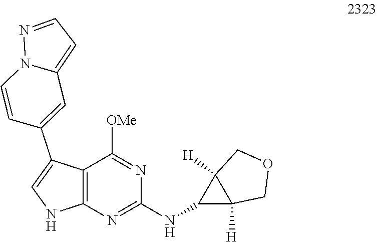

N-((lR,5 S,6r)-3-Oxabicyclo [3 .1. 0] hexan-6-yl)-4-methoxy-5-(pyrazolo [1,5-a] pyridin-5-yl)-7H-pyrrolo [2,3-d] pyrimidin-2-amine 2323.

White solid (9 mg, 0.025 mmol, 35.6% yield). ¹H NMR (499 MHz, DMSO-d₆) δppm 1.83 (2 H, s), 2.52-2.55 (1 H, m), 3.66 (2 H, d, J=7.94 Hz), 3.88 (2 H, d, J=8.21 Hz), 3.99 (3 H, s), 6.54 (1 H, d, J=1.37 Hz), 6.94 (1 H, d, J=3.29 Hz), 7.19 (1 H, dd, J=7.26, 2.05 Hz), 7.37 (1 H, d, J=2.46 Hz), 7.93 (1 H, d, J=2.19 Hz), 7.99 (1 H, d, J=1.09 Hz), 8.58 (1 H, d, J=7.12 Hz), 11.58 (1 H, br s); ESIMS found for C₁₉1-1₁₈N₆O₂ m/z 363.2 (M+1).

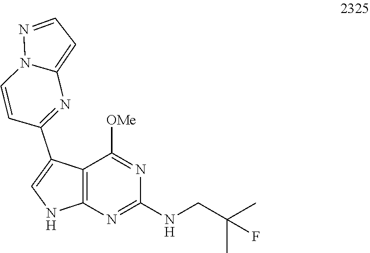 [SMILES: COc1nc(NCC(C)(C)F)nc2[nH]cc(-c3ccn4nccc4n3)c12]

N-(2-Fluoro-2-methylpropyl)-4-methoxy-5-(pyrazolo [1,5-a]pyrimidin-5-yl)-7H-pyrrolo [2,3-d]pyrimidin-2-amine 2325.

Beige solid (2 mg, 0.006 mmol, 14.3% yield). ¹H NMR (499 MHz, DMSO-d₆) δppm 1.35 (6 H, d, J=21.40 Hz), 3.58 (2 H, dd, J=18.62, 6.57 Hz), 4.02 (3 H, s), 6.54 (1 H, dd, J=2.19, 0.82 Hz), 6.88 (1 H, br t, J=6.30 Hz), 7.64 (1 H, s), 7.67 (1 H, d, J=7.39 Hz), 8.12 (1 H, d, J=2.19 Hz), 8.98 (1 H, dd, J=7.39, 0.82 Hz), 11.83 (1 H, br s); ESIMS found for C₁₇HisFN₇O m/z 356.2 (M+1).

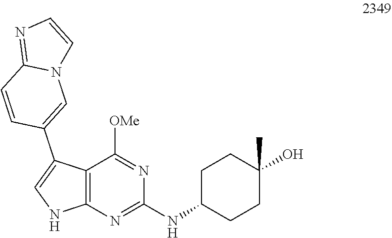 [SMILES: COc1nc(N[C@H]2CC[C@@](C)(O)CC2)nc2[nH]cc(-c3ccc4nccn4c3)c12]

cis-4-45-(Imidazo [1,2-a]pyridin-6-yl)-7H-pyrrolo [2,3-d]pyrimidin-2-yl)amino)-1-methylcyclohexan-1-ol 2349.

White solid (853.79 mg, 2.355 mmol). ¹H NMR (400 MHz, DMSO-d₆) δppm 1.12 (3 H, s), 1.29-1.42 (2 H, m), 1.59 (2 H, br d, J=12.76 Hz), 1.61-1.77 (4 H, m), 3.67 (1 H, br s), 4.06 (1 H, s), 6.56 (1 H, br d, J=6.38 Hz), 7.50 (1 H, br s), 7.54 (1 H, br s), 7.55-7.64 (2 H, m), 7.96 (1 H, s), 8.90 (1 H, br s), 8.98 (1 H, s), 11.47 (1 H, br s); ESIMS found for C201⁻122N60 m/z 363.2 (M+1).

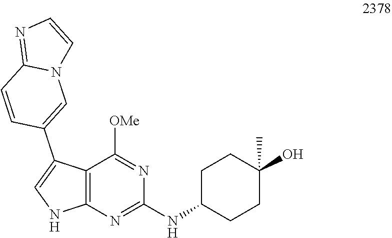 [SMILES: COc1nc(N[C@H]2CC[C@](C)(O)CC2)nc2[nH]cc(-c3ccc4nccn4c3)c12]

trans-4-45-(Imidazo[1,2-a]pyridin-6-yl)-4-methoxy-7H-pyrrolo[2,3-d]pyrimidin-2-yl)amino)-1-methylcyclohexan-1-ol 2378.

White solid (80.0 mg, 0.204 mmol). ¹H NMR (400 MHz, DMSO-d₆) δppm 1.15 (3 H, s), 1.34-1.51 (4 H, m), 1.60 (2 H, br dd, J=8.00, 5.13 Hz), 1.80-1.93 (2 H, m), 3.71-3.85 (1 H, m), 3.95 (3 H, s), 4.23 (1 H, s), 6.44 (1 H, d, J=7.88 Hz), 7.17 (1 H, d, J=2.38 Hz), 7.49-7.52 (2 H, m), 7.53 (1 H, d, J=1.13 Hz), 7.95 (1 H, s), 8.77 (1 H, s), 11.45 (1 H, br s) ESIMS found for C2J⁻124N602 m/z 393.2 (M+1).

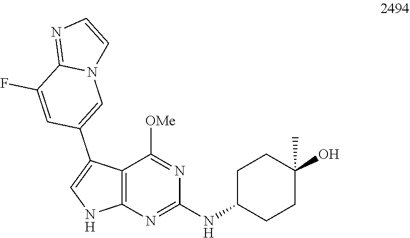 [SMILES: COc1nc(N[C@H]2CC[C@](C)(O)CC2)nc2[nH]cc(-c3cc(F)c4nccn4c3)c12]

trans-4-((5-(8-Fluoroimidazo[1,2-a]pyridin-6-yl)-4-methoxy-7H-pyrrolo[2,3-d]pyrimidin-2-yl)amino)-1-methylcyclohexan-1-ol 2494.

White solid (380.0 mg, 0.926 mmol). ¹H NMR (400 MHz, DMSO-d₆) δppm 1.15 (3 H, s), 1.34-1.52 (4 H, m), 1.60 (2 H, br dd, J=8.25, 5.00 Hz), 1.79-1.92 (2 H, m), 3.79 (1 H, br s), 3.97 (3 H, s), 4.23 (1 H, s), 6.47 (1 H, d, J=8.00 Hz), 7.26 (1 H, d, J=2.38 Hz), 7.45 (1 H, dd, J=12.88, 1.13 Hz), 7.59 (1 H, d, J=1.00 Hz), 8.11 (1 H, dd, J=3.06, 0.94 Hz), 8.72 (1 H, d, J=1.25 Hz), 11.51 (1 H, br s); ESIMS found for C_{2I}F1₂₃FN₆O₂ m/z 411.2 (M+1).

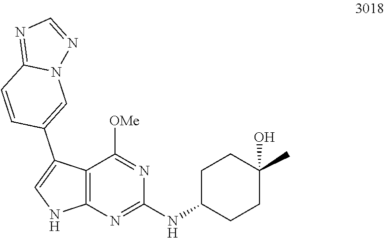 [SMILES: COc1nc(N[C@H]2CC[C@@](C)(O)CC2)nc2[nH]cc(-c3ccc4ncnn4c3)c12]

(1s,4s)-4-45-([1,2,4]Triazolo [1,5-a]pyridin-6-yl)-7H-pyrrolo [2,3-d]pyrimidin-2-yl)amino)-1-methylcyclohexan-1-ol 3018.

White solid (365.82 mg, 1.007 mmol). ¹H NMR (400 MHz, DMSO-d₆) δppm 1.13 (3 H, s), 1.36 (2 H, td, J=12.69, 4.75 Hz), 1.59 (2 H, br d, J=13.88 Hz), 1.62-1.74 (4 H, m), 3.59-3.75 (1 H, m), 3.99 (1 H, s), 6.62 (1 H, br d, J=6.63 Hz), 7.62 (1 H, s), 7.87 (1 H, d, J=9.26 Hz), 8.05 (1 H, dd, J=9.26, 1.63 Hz), 8.48 (1 H, s), 8.93 (1 H, s), 9.17 (1 H, s), 11.59 (1 H, br s); ESIMS found for C₁₉H₂iN70 m/z 364.1 (M+1).

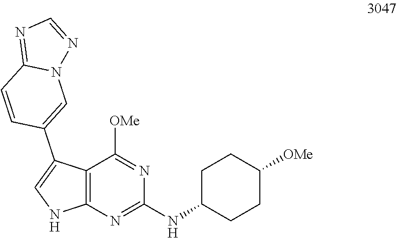

5-([1,2,4]Triazolo[1,5-a]pyridin-6-yl)-4-methoxy-N-(cis-4-methoxycyclohexyl)-7H-pyrrolo[2,3-d]pyrimidin-2-amine 3047.

Off-white solid (6 mg, 0.015 mmol, 19.9% yield). ¹1-1 NMR (499 MHz, DMSO-d₆) δppm 1.44-1.53 (2 H, m), 1.54-1.64 (2 H, m), 1.64-1.73 (2 H, m), 1.81-1.90 (2 H, m), 3.22 (3 H, s), 3.34-3.38 (1 H, m), 3.73-3.86 (1 H, m), 3.96 (3 H, s), 6.55 (1 H, d, J=7.67 Hz), 7.34 (1 H, d, J=2.19 Hz), 7.81 (1 H, d, J=9.31 Hz), 7.98 (1 H, dd, J=9.03, 1.64 Hz), 8.45 (1 H, s), 9.15 (1 H, s), 11.56 (1 H, br s); ESIMS found for C₂oH₂₃N₇O₂ m/z 394.2 (M+1).

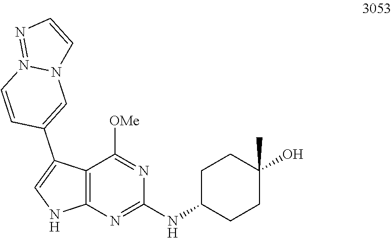

cis-44(54[1,2,4] Triazolo [1,5-a]pyridin-6-yl)-4-methoxy-7H-pyrrolo [2,3-d]pyrimidin-2-yDamino)-1-methylcyclohexan-1-ol 3053.

White solid (21 mg, 0.053 mmol, 32.5% yield). ¹H NMR (499 MHz, DMSO-d₆) δppm 1.12 (3 H, s), 1.35 (2 H, td, J=12.87, 4.65 Hz), 1.58 (2 H, br d, J=12.32 Hz), 1.62-1.73 (4 H, m), 3.61-3.71 (1 H, m), 3.96 (3 H, s), 3.99 (1 H, br s), 6.48 (1 H, d, J=7.67 Hz), 7.33 (1 H, d, J=2.19 Hz), 7.77-7.88 (1 H, m), 7.98 (1 H, dd, J=9.31, 1.64 Hz), 8.45 (1 H, s), 9.15 (1 H, s), 11.55 (1 H, br s); ESIMS found for C₂₀H₂₃N₇O₂ m/z 394.2 (M+1).

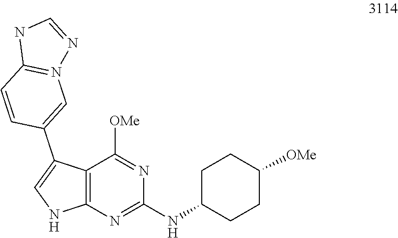

4-Methoxy-N-(cis-4-methoxycyclohexyl)-5-(1-methyl-1H-benzo[d] [1,2,3]triazol-6-yl)-7H-pyrrolo [2,3-d]pyrimidin-2-amine 3114.

White solid (16 mg, 0.039 mmol, 49.0% yield). ¹H NMR (499 MHz, DMSO-d₆) δppm 1.44-1.53 (2 H, m), 1.55-1.65 (2 H, m), 1.65-1.73 (2 H, m), 1.82-1.89 (2 H, m), 3.23 (3 H, s), 3.30-3.35 (1 H, m), 3.73-3.84 (1 H, m), 3.95 (3 H, s), 4.29 (3 H, s), 6.50 (1 H, d, J=7.67 Hz), 7.23 (1 H, d, J=2.46 Hz), 7.69 (1 H, dd, J=8.62, 1.23 Hz), 7.93 (1 H, d, J=8.76 Hz), 8.03 (1 H, s), 11.50 (1 H, br s); ESIMS found for C₂₁H₂₅N₇O₂ m/z 408.2 (M+1).

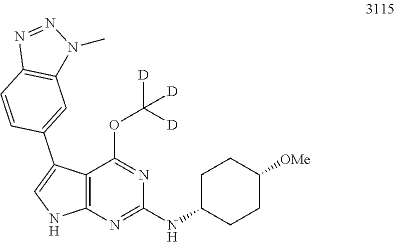

4-(Methoxy-d₃)-N-(cis-4-methoxycyclohexyl)-5-(1-methyl-1H-benzo[d] [1,2,3]triazol-6-yl)-7H-pyrrolo [2,3-d]pyrimidin-2-amine 3115.

White solid (55.77 mg, 0.136 mmol). ¹H NMR (400 MHz, DMSO-d₆) δppm 1.42-1.53 (2 H, m), 1.54-1.65 (2 H, m), 1.65-1.73 (2 H, m), 1.79-1.94 (2 H, m), 3.22 (3 H, s), 3.24-3.30 (1 H, m), 3.70-3.87 (1 H, m), 4.29 (3 H, s), 6.47 (1 H, br d, J=7.63 Hz), 7.23 (1 H, d, J=1.88 Hz), 7.69 (1 H, d, J=8.63 Hz), 7.93 (1 H, d, J=8.75 Hz), 8.03 (1 H, s), 11.48 (1 H, br s); ESIMS found for C_{2I}F1₂₂[2H₃]N₇O₂ m/z 411.2 (M+1).

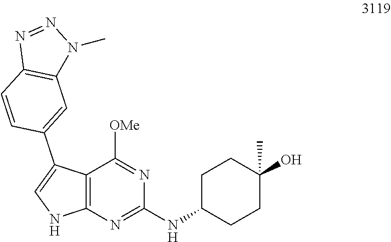 [SMILES: COc1nc(N[C@H]2CC[C@](C)(O)CC2)nc2[nH]cc(-c3ccc4nnn(C)c4c3)c12]

trans-4-44-Methoxy-5-(1-methyl-1H-benzo[d] [1,2,3]triazol-6-yl)-7H-pyrrolo [2,3-d]pyrimidin-2-yl)amino)-1-methylcyclohexan-1-ol 3119.

White solid (130.0 mg, 0.319 mmol). ¹H NMR (400 MHz, DMSO-d₆) δppm 1.16 (3 H, s), 1.36-1.51 (4 H, m), 1.60 (2 H, br dd, J=7.95, 5.01 Hz), 1.81-1.94 (2 H, m), 3.80 (1 H, dt, J=5.38, 2.93 Hz), 3.96 (3 H, s), 4.29 (3 H, s), 6.54 (1 H, br s), 7.25 (1 H, d, J=2.32 Hz), 7.69 (1 H, dd, J=8.74, 1.41 Hz), 7.94 (1 H, d, J=8.68 Hz), 8.03 (1 H, s), 11.55 (1 H, br s); ESIMS found for C211125N702 m/z 408.2 (M+1).

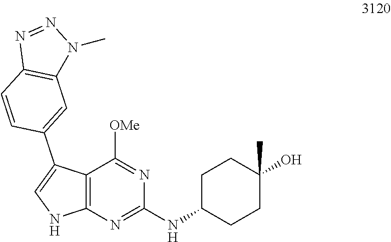 [SMILES: COc1nc(N[C@H]2CC[C@@](C)(O)CC2)nc2[nH]cc(-c3ccc4nnn(C)c4c3)c12]

cis-4-((4-Methoxy-5-(1-methyl-1H-benzo[d][1,2,3]triazol-6-yl)-7H-pyrrolo [2,3-d]pyrimidin-2-yl)amino)-1-methylcyclohexan-1-ol 3120.

White solid (25 mg, 0.061 mmol, 35.5% yield). ¹H NMR (499 MHz, DMSO-d₆) δppm 1.13 (3 H, s), 1.36 (2 H, td, J=12.94, 4.52 Hz), 1.59 (2 H, br d, J=12.05 Hz), 1.63-1.74 (4 H, m), 3.63-3.73 (1 H, m), 3.95 (3 H, s), 4.00 (1 H, br s), 4.29 (3 H, s), 6.42 (1 H, d, J=7.94 Hz), 7.23 (1 H, d, J=2.19 Hz), 7.69 (1 H, dd, J=8.76, 1.37 Hz), 7.93 (1 H, d, J=8.49 Hz), 8.03 (1 H, s), 11.49 (1 H, br s); ESIMS found for C₂it1₂₅N₇O₂ m/z 408.2 (M+1).

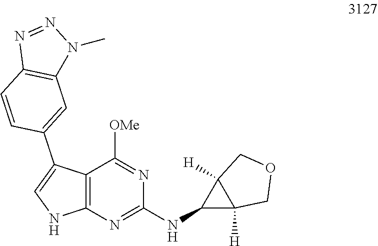 [SMILES: COc1nc(N[C@@H]2[C@@H]3COC[C@@H]32)nc2[nH]cc(-c3ccc4nnn(C)c4c3)c12]

N-((lR,5 S,6s)-3-Oxabicyclo [3 .1.0] hexan-6-yl)-4-methoxy-5-(1-methyl-1H-benzo[d][1,2,3]triazol-6-yl)-7H-pyrrolo[2,3-d]pyrimidin-2-amine 3127.

Brown solid (8 mg, 0.021 mmol, 17.3% yield). ¹H NMR (499 MHz, DMSO-d₆) δppm 1.83 (2 H, s), 2.51-2.56 (1 H, m), 3.67 (2 H, br d, J=8.21 Hz), 3.89 (2 H, d, J=8.21 Hz), 3.97 (3 H, s), 4.30 (3 H, s), 6.92 (1 H, d, J=3.56 Hz), 7.28 (1 H, d, J=2.46 Hz), 7.70 (1 H, dd, J=8.76, 1.37 Hz), 7.94 (1 H, d, J=8.76 Hz), 8.04 (1 H, s), 11.55 (1 H, br s); ESIMS found for C₁₉H₁₉N702 m/z 378.2 (M+1).

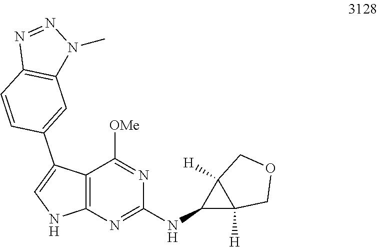 [SMILES: COc1nc(N[C@@H]2[C@@H]3COC[C@@H]32)nc2[nH]cc(-c3ccc4nnn(C)c4c3)c12]

N-((lR,5 S,6r)-3-Oxabicyclo [3 .1. 0] hexan-6-yl)-4-methoxy-5-(1-methyl-1H-benzo[d][1,2,3]triazol-6-yl)-7H-pyrrolo[2,3-d]pyrimidin-2-amine 3128.

White solid (13 mg, 0.034 mmol, 41.5% yield). ¹H NMR (499 MHz, DMSO-d₆) δppm 1.83 (2 H, s), 2.52-2.55 (1 H, m), 3.66 (2 H, d, J=7.94 Hz), 3.89 (2 H, d, J=8.21 Hz), 3.97 (3 H, s), 4.30 (3 H, s), 6.92 (1 H, d, J=3.56 Hz), 7.28 (1 H, d, J=2.19 Hz), 7.70 (1 H, dd, J=8.62, 1.51 Hz), 7.94 (1 H, d, J=8.76 Hz), 8.04 (1 H, s), 11.55 (1 H, br s); ESIMS found for C₁₉H₁₉N702 m/z 378.2 (M+1).

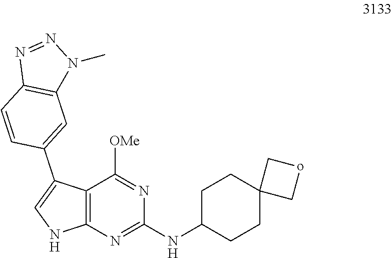

4-Methoxy-5-(1-methyl-1H-benzo[d][1,2,3]triazol-6-yl)-N-(2-oxaspiro [3 .5]nonan-7-yl)-7H-pyrrolo [2,3-d] pyrimidin-2-amine 3133.

Beige solid (12 mg, 0.029 mmol, 20.5% yield). ¹H NMR (499 MHz, DMSO-d₆) δppm 1.19-1.35 (2 H, m), 1.51 (2 H, td, J=12.87, 3.01 Hz), 1.79-1.92 (2 H, m), 2.08 (2 H, br d, J=12.87 Hz), 3.65-3.75 (1 H, m), 3.94 (3 H, s), 4.24 (2 H, s), 4.29 (3 H, s), 4.32 (2 H, s), 6.45 (1 H, d, J=7.94 Hz), 7.24 (1 H, d, J=2.46 Hz), 7.68 (1 H, dd, J=8.76, 1.37 Hz), 7.93 (1 H, d, J=8.76 Hz), 8.03 (1 H, s), 11.51 (1 H, br s); ESIMS found for C₂₂H₂₅N₇O₂ m/z 420.2 (M+1).

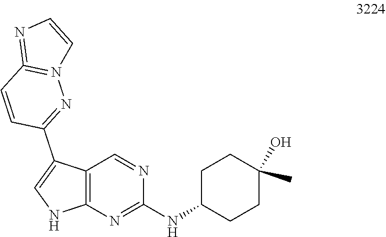

(1s,4s)-4-((5-(Imidazo[1,2-b]pyridazin-6-yl)-7H-pyrrolo[2,3-d]pyrimidin-2-yl)amino)-1-methylcyclohexan-1-ol 3224.

White solid (127.11 mg, 0.350 mmol). ¹H NMR (400 MHz, DMSO-d₆) δppm 1.13 (3 H, s), 1.36 (2 H, td, J=12.56, 5.32 Hz), 1.59 (2 H, br d, J=14.79 Hz), 1.62-1.73 (4 H, m), 3.59-3.75 (1 H, m), 4.00 (1 H, s), 6.63-6.81 (1 H, m), 7.68 (1 H, d, J=1.22 Hz), 7.73 (1 H, d, J=9.66 Hz), 8.01 (1 H, d, J=2.69 Hz), 8.04 (1 H, dd, J=9.54, 0.61 Hz), 8.29 (1 H, s), 9.25 (1 H, s), 11.78 (1 H, br s); ESIMS found for C₁₉H₂rN₇O m/z 364.1 (M+1).

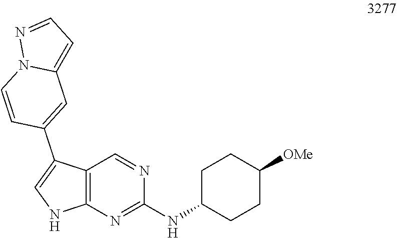 [SMILES: CO[C@H]1CC[C@H](Nc2ncc3c(-c4ccn5nccc5c4)c[nH]c3n2)CC1]

N-(trans-4-Methoxycyclohexyl)-5-(pyrazolo[1,5-a]pyridin-5-yl)-7H-pyrrolo [2,3-d]pyrimidin-2-amine 3277.

Beige solid (8 mg, 0.022 mmol, 11.6% yield). ¹H NMR (499 MHz, DMSO-d₆) δppm 1.15-1.26 (2 H, m), 1.27-1.37 (2 H, m), 2.01 (4 H, br t, J=15.74 Hz), 3.06-3.18 (1 H, m), 3.24 (3 H, s), 3.65-3.80 (1 H, m), 6.55 (1 H, d, J=1.64 Hz), 6.72 (1 H, br d, J=6.84 Hz), 7.24 (1 H, dd, J=7.39, 1.92 Hz), 7.66 (1 H, s), 7.94 (1 H, d, J=2.19 Hz), 7.97 (1 H, d, J=1.37 Hz), 8.61 (1 H, d, J=7.39 Hz), 9.03 (1 H, s), 11.51 (1 H, br s); ESIMS found for C₂₀I-1₂₂N₆O m/z 363.2 (M+1).

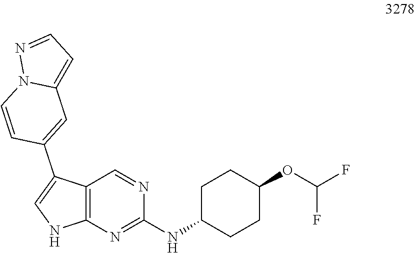 [SMILES: FC(F)O[C@H]1CC[C@H](Nc2ncc3c(-c4ccn5nccc5c4)c[nH]c3n2)CC1]

N-(trans-4-(Difluoromethoxy)cyclohexyl)-5-(pyrazolo [1,5-a]pyridin-5-yl)-7H-pyrrolo [2,3-d]pyrimidin-2-amine 3278.

White solid (14 mg, 0.035 mmol, 18.9% yield). ¹H NMR (499 MHz, DMSO-d₆) δppm 1.33-1.43 (2 H, m), 1.44-1.53 (2 H, m), 1.95-2.07 (4 H, m), 3.69-3.81 (1 H, m), 4.01 -4.11 (1 H, m), 6.55 (1 H, d, J=1.64 Hz), 6.73 (2 H, t, J=76.80 Hz), 6.76 (1 H, br d, J=5 .7 5 Hz), 7.24 (1 H, dd, J=7.26, 2.05 Hz), 7.68 (1 H, d, J=2.46 Hz), 7.94 (1 H, d, J=2.19 Hz), 7.97 (1 H, d, J=1.37 Hz), 8.61 (1 H, d, J=7.39 Hz), 9.04 (1 H, s), 11.58 (1 H, br s); ESIMS found for C₂₀I-1₂₀F₂N₆O m/z 399.2 (M+1).

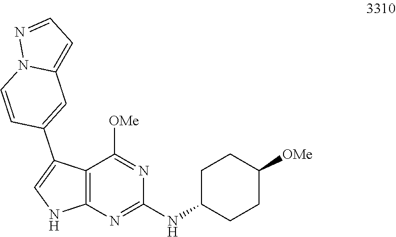 [SMILES: COc1nc(N[C@H]2CC[C@H](OC)CC2)nc2[nH]cc(-c3ccn4nccc4c3)c12]

4-Methoxy-N-(trans-4-methoxycyclohexyl)-5-(pyrazolo [1,5-a] pyridin-5-yl)-7H-pyrrolo [2,3-d]pyrimidin-2-amine 3310.

White solid (19 mg, 0.048 mmol, 64.9% yield). ¹H NMR (499 MHz, DMSO-d₆) δppm 1.12-1.25 (2 H, m), 1.27-1.39 (2 H, m), 1.93-2.08 (4 H, m), 3.06-3.16 (1 H, m), 3.24 (3 H, s), 3.72 (1 H, tdt, J=11.05, 11.05, 7.39, 3.87, 3.87 Hz), 3.97 (3 H, s), 6.52 (1 H, d, J=7.94 Hz), 6.54 (1 H, dd, J=2.33, 0.68 Hz), 7.18 (1 H, dd, J=7.26, 2.05 Hz), 7.32 (1 H, d, J=2.19 Hz), 7.93 (1 H, d, J=2.19 Hz), 7.98 (1 H, d, J=1.37 Hz), 8.57 (1 H, d, J=7.39 Hz), 11.56 (1 H, br s); ESIMS found for C_{2I}F1₂₄N₆O₂ m/z 393.2 (M+1).

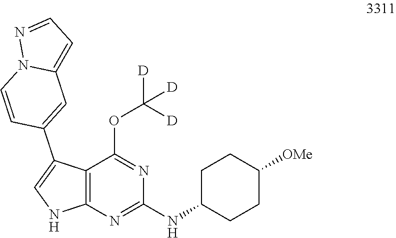 [SMILES: [2H]C([2H])([2H])Oc1nc(N[C@H]2CC[C@@H](OC)CC2)nc2[nH]cc(-c3ccn4nccc4c3)c12]

4-(Methoxy-d₃)-N-(cis-4-methoxycyclohexyl)-5-(pyrazolo[1,5-a]pyridin-5-yl)-7H-pyrrolo[2,3-d]pyrimidin-2-amine 3311.

White solid (33.31 mg, 0.084 mmol). ¹H NMR (400 MHz, DMSO-d₆) δppm 1.43-1.54 (2 H, m), 1.54-1.65 (2 H, m), 1.66-1.75 (2 H, m), 1.80-1.90 (2 H, m), 3.22(3 H, s), 3.31-3.38 (1 H, m), 3.73-3.87 (1 H, m), 6.55 (1 H, d, J=1.71 Hz), 7.17 (1 H, dd, J=7.34, 1.96 Hz), 7.33 (1 H, d, J=2.20 Hz), 7.93 (1 H, d, J=2.20 Hz), 7.98 (1 H, d, J=1.22 Hz), 8.58 (1 H, d, J=7.34 Hz), 11.60 (1 H, br s); ESIMS found for C_{2I}I-1₂₁[2H₃]1\1₆0₂ m/z 396.2 (M+1).

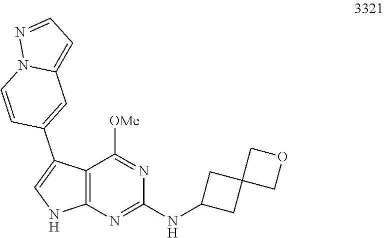 [SMILES: COc1nc(NC2CC3(COC3)C2)nc2[nH]cc(-c3ccn4nccc4c3)c12]

4-Methoxy-5-(pyrazolo[1,5-a]pyridin-5-yl)-N-(2-oxaspiro[3.3]heptan-6-yl)-7H-pyrrolo[2,3-d]pyrimidin-2-amine 3321.

Light brown solid (6 mg, 0.016 mmol, 19.1% yield). ESIMS found for

C201120N602 m/z 377.2 (M+1).

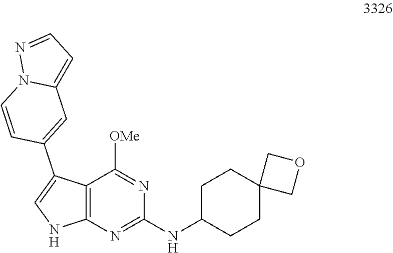 [SMILES: COc1nc(NC2CCC3(CC2)COC3)nc2[nH]cc(-c3ccn4nccc4c3)c12]

4-Methoxy-5-(pyrazolo [1,5-a]pyridin-5-yl)-N-(2-oxaspiro [3 .5] nonan-7-yl)-7H-pyrrolo [2,3-d]pyrimidin-2-amine 3326.

Beige solid (10 mg, 0.025 mmol, 19.7% yield). ¹H NMR (499 MHz, DMSO-d₆) δppm 1.19-1.33 (2 H, m), 1.51 (2 H, td, J=12.94, 3.42 Hz), 1.82-1.92 (2 H, m), 2.07 (2 H, br d, J=13.14 Hz), 3.64-3.76 (1 H, m), 3.96 (3 H, s), 4.24 (2 H, s), 4.32 (2 H, s), 6.46 (1 H, d, J=7.94 Hz), 6.54 (1 H, d, J=1.64 Hz), 7.18 (1 H, dd, J=7.39, 1.92 Hz), 7.32 (1 H, d, J=2.19 Hz), 7.92 (1 H, d, J=2.19 Hz), 7.98 (1 H, d, J=1.37 Hz), 8.57 (1 H, d, J=7.39 Hz), 11.54 (1 H, br s); ESIMS found for C22H₂₄N602 m/z 405.2 (M+1).

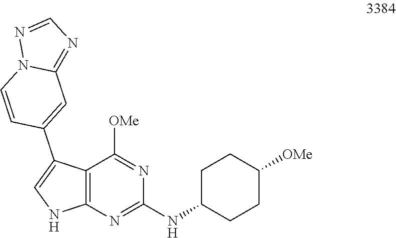 [SMILES: COc1nc(N[C@H]2CC[C@@H](OC)CC2)nc2[nH]cc(-c3ccn4ncnc4c3)c12]

5-([1,2,4]Triazolo[1,5-a]pyridin-7-yl)-4-methoxy-N-(cis-4-methoxycyclohexyl)-7H-pyrrolo[2,3-d]pyrimidin-2-amine 3384.

Off-white solid (10.0 mg, 0.025 mmol, 18.6% yield). ¹H NMR (499 MHz, DMSO-d₆) δppm 1.43-1.53 (2 H, m), 1.54-1.64 (2 H, m), 1.65-1.73 (2 H, m), 1.81-1.91 (2 H, m), 3.22 (3 H, s), 3.74-3.85 (1 H, m), 3.99 (3 H, s), 6.60 (1 H, d, J=7.67 Hz), 7.51 (1 H, d, J=2.19 Hz), 7.52 (1 H, dd, J=7.26, 1.78 Hz), 8.12 (1 H, d, J=0.82 Hz), 8.40 (1 H, s), 8.83 (1 H, d, J=7.12 Hz), 11.69 (1 H, br s); ESIMS found for C₂oH₂₃N₇O₂ m/z 394.2 (M+1).

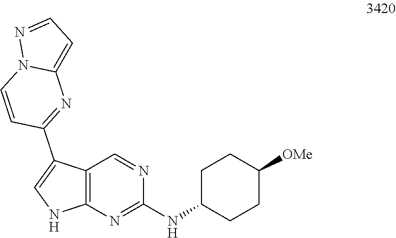 [SMILES: CO[C@H]1CC[C@H](Nc2ncc3c(-c4ccn5nccc5n4)c[nH]c3n2)CC1]

N-(trans-4-Methoxycyclohexyl)-5-(pyrazolo[1,5-a]pyrimidin-5-yl)-7H-pyrrolo [2,3-d] pyrimidin-2-amine 3420.

Beige solid (4 mg, 0.011 mmol, 13.5% yield). ¹H NMR (499 MHz, DMSO-d₆) δppm 1.12-1.25 (2 H, m), 1.27-1.38 (2 H, m), 2.01 (4 H, brt, J=15.74 Hz), 3.13 (1 H, tt, J=10.16, 3.80 Hz), 3.24 (3 H, s), 3.63-3.80 (1 H, m), 6.60 (1 H, d, J=1.92 Hz), 6.81 (1 H, br d, J=1.92 Hz), 7.46 (1 H, d, J=7.39 Hz), 8.10 (1 H, d, J=2.19 Hz), 8.11 (1 H, d, J=2.46 Hz), 8.95 (1 H, d, J=7.39 Hz), 9.32 (1 H, s), 11.63 (1 H, br s); ESIMS found for C₁₉H₂ᵢN₇O m/z 364.2 (M+1).

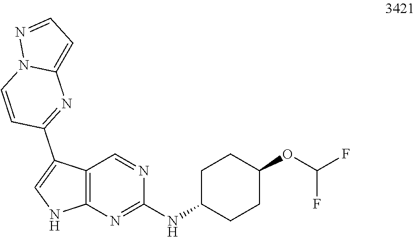 [SMILES: FC(F)O[C@H]1CC[C@H](Nc2ncc3c(-c4ccn5nccc5n4)c[nH]c3n2)CC1]

N-(trans-4-(difluoromethoxy)cyclohexyl)-5-(pyrazolo[1,5-a]pyrimidin-5-yl)-7H-pyrrolo[2,3-d]pyrimidin-2-amine 3421.

White solid (5 mg, 0.013 mmol, 52.0% yield). ¹H NMR (499 MHz, DMSO-d₆) δppm 1.33-1.43 (2 H, m), 1.44-1.54 (2 H, m), 2.01 (4 H, br d, J=11.50 Hz), 3.70-3.80 (1 H, m), 4.01-4.12 (1 H, m), 6.72 (2 H, t, J=76.80 Hz), 6.60 (1 H, d, J=1.64 Hz), 6.85 (1 H, br s), 7.47 (1 H, d, J=7.39 Hz), 8.10 (1 H, d, J=2.19 Hz), 8.12 (1 H, d, J=2.46 Hz), 8.91-9.00 (1 H, m), 9.33 (1 H, s), 11.90 (1 H, br s); ESIMS found for C₁₉H₁₉F₂N₇O m/z 400.2 (M+1).

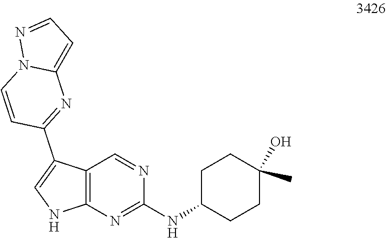 [SMILES: C[C@]1(O)CC[C@@H](Nc2ncc3c(-c4ccn5nccc5n4)c[nH]c3n2)CC1]

(1s,4s)-1-Methyl-4-((5-(pyrazolo [1,5-a] pyrimidin-5-yl)-7H-pyrrolo [2,3-d]pyrimidin-2-yl)amino)cyclohexan-1-ol 3426.

White solid (427.0 mg, 1.175 mmol). ¹H NMR (400 MHz, DMSO-d₆) δppm 1.13 (3 H, s), 1.36 (2 H, td, J=12.51, 4.88 Hz), 1.59 (2 H, br d, J=14.01 Hz), 1.62-1.73 (4 H, m), 3.58-3.76 (1 H, m), 3.99 (1 H, s), 6.59 (1 H, d, J=1.88 Hz), 6.65-6.78 (1 H, m), 7.46 (1 H, d, J=7.38 Hz), 8.09 (1 H, s), 8.10 (1 H, s), 8.94 (1 H, d, J=7.50 Hz), 9.31 (1 H, s), 11.84 (1 H, br s); ESIMS found for C₁₉H₂iN70 m/z 364.1 (M+1).

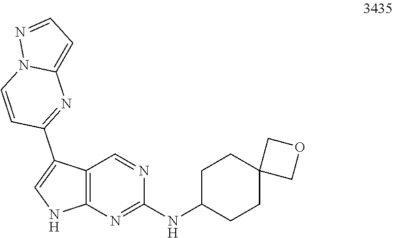 [SMILES: c1cc2nc(-c3c[nH]c4nc(NC5CCC6(CC5)COC6)ncc34)ccn2n1]

5-(Pyrazolo [1,5-a]pyrimidin-5-yl)-N-(2-oxaspiro [3 .5]nonan-7-yl)-7H-pyrrolo [2,3-d] pyrimidin-2-amine 3435.

White solid (21 mg, 0.056 mmol, 39.0% yield). ¹H NMR (499 MHz, DMSO-d₆) δppm 1.21-1.30 (2 H, m), 1.52 (2 H, td, J=12.87, 3.29 Hz), 1.86 (2 H, br dd, J=13.00, 3.15 Hz), 2.07 (2 H, br d, J=12.87 Hz), 3.63-3.75 (1 H, m), 4.24 (2 H, s), 4.32 (2 H, s), 6.60 (1 H, d, J=1.92 Hz), 6.75 (1 H, br s), 7.46 (1 H, d, J=7.67 Hz), 8.10 (1 H, d, J=2.19 Hz), 8.11 (1 H, d, J=2.46 Hz), 8.95 (1 H, d, J=7.39 Hz), 9.32 (1 H, s), 11.87 (1 H, br s); ESIMS found for C₂₀H_{2I}N₇O m/z 376.2 (M+1).

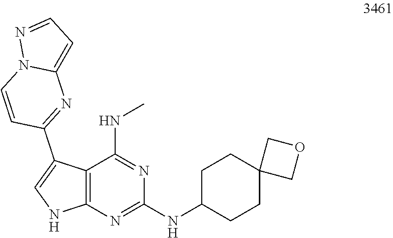 [SMILES: CNc1nc(NC2CCC3(CC2)COC3)nc2[nH]cc(-c3ccn4nccc4n3)c12]

N⁴-Methyl-5-(pyrazolo [1,5-a]pyrimidin-5-yl)-N²-(2-oxaspiro [3 .5] nonan-7-yl)-7H-pyrrolo [2,3-d] pyrimidine -2,4-diamine 3461.

Yellow solid (7 mg, 0.017 mmol, 60.4% yield). ¹H NMR (499 MHz, DMSO-d₆) δppm 1.19-1.30 (2 H, m), 1.49 (2 H, td, J=12.87, 2.74 Hz), 1.84 (2 H, br dd, J=12.87, 2.74 Hz), 2.06 (2 H, br d, J=12.87 Hz), 3.03 (3 H, d, J=4.65 Hz), 3.58-3.75 (1 H, m), 4.23 (2 H, s), 4.31 (2 H, s), 5.87-6.02 (1 H, m), 6.62 (1 H, d, J=1.37 Hz), 7.49 (1 H, d, J=7.67 Hz), 7.91 (1 H, br s), 8.10 (1 H, d, J=2.19 Hz), 8.91 (1 H, d, J=7.67 Hz), 9.95 (1 H, br s), 11.63 (1 H, br s); ESIMS found for C₂₁H₂₄N80 m/z 405.25 (M+1).

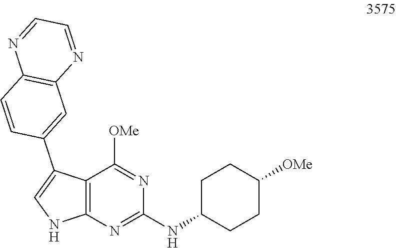 [SMILES: COc1nc(N[C@H]2CC[C@@H](OC)CC2)nc2[nH]cc(-c3ccc4nccnc4c3)c12]

4-Methoxy-N-(cis-4-methoxycyclohexyl)-5-(quinoxalin-6-yl)-7H-pyrrolo [2,3-d] pyrimidin-2-amine 3575.

Yellow solid (5 mg, 0.012 mmol, 34.5% yield). ¹H NMR (499 MHz, DMSO-d₆) δppm 1.44-1.54 (2 H, m), 1.56-1.65 (2 H, m), 1.66-1.73 (2 H, m), 1.80-1.92 (2 H, m), 3.23 (3 H, s), 3.33-3.37 (1 H, m), 3.74-3.86 (1 H, m), 3.97 (3 H, s), 6.55 (1 H, d, J=7.67 Hz), 7.43 (1 H, d, J=2.46 Hz), 8.02 (1 H, d, J=8.76 Hz), 8.19 (1 H, dd, J=8.76, 1.92 Hz), 8.41 (1 H, d, J=1.92 Hz), 8.84 (1 H, d, J=1.64 Hz), 8.89 (1 H, d, J=1.92 Hz), 11.62 (1 H, br s); ESIMS found for

C₂₂H₂₄N₆O₂ m/z 405.2 (M+1).

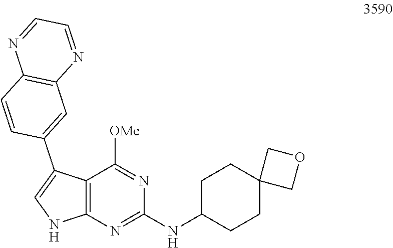 [SMILES: COc1nc(NC2CCC3(CC2)COC3)nc2[nH]cc(-c3ccc4nccnc4c3)c12]

4-Methoxy-5-(quinoxalin-6-yl)-N-(2-oxaspiro [3 .5]nonan-7-yl)-7H-pyrrolo [2,3-d] pyrimidin-2-amine 3590.

Yellow solid (12 mg, 0.029 mmol, 22.5% yield). ¹H NMR (499 MHz, DMSO-d₆) δppm 1.18-1.36 (2 H, m), 1.52 (2 H, td, J=13.00, 3.29 Hz), 1.80-1.93 (2 H, m), 2.08 (2 H, br d, J=13.14 Hz), 3.70 (1 H, tdt, J=10.87, 10.87, 7.43, 3.90, 3.90 Hz), 3.96 (3 H, s), 4.24 (2 H, s), 4.33 (2 H, s), 6.50 (1 H, d, J=7.94 Hz), 7.43 (1 H, d, J=2.46 Hz), 8.02 (1 H, d, J=8.76 Hz), 8.18 (1 H, dd, J=8.76, 1.92 Hz), 8.40 (1 H, d, J=1.92 Hz), 8.84 (1 H, d, J=1.92 Hz), 8.89 (1 H, d, J=1.92 Hz), 11.64 (1 H, br s); ESIMS found for C₂₃H₂₄N₆O₂ m/z 417.2 (M+1).

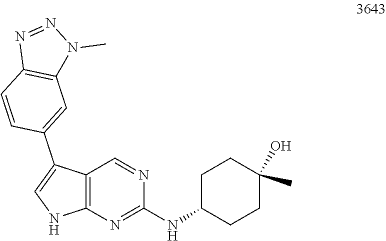 [SMILES: Cn1nnc2ccc(-c3c[nH]c4nc(N[C@H]5CC[C@@](C)(O)CC5)ncc34)cc21]

(1s,4s)-1-Methyl-4-45-(1-methyl-1H-benzo[d][1,2,3]triazol-6-yl)-7H-pyrrolo[2,3-d]pyrimidin-2-yl)amino)cyclohexan-1-ol 3643.

White solid (397.64 mg, 1.053 mmol). ¹H NMR (400 MHz, DMSO-d₆) δppm 1.13 (3 H, s), 1.36 (2 H, td, J=12.69, 4.71 Hz), 1.59 (2 H, br d, J=14.55 Hz), 1.63-1.74 (4 H, m), 3.61-3.76 (1 H, m), 4.00 (1 H, s), 4.35 (3 H, s), 6.59 (1 H, br d, J=7.34 Hz), 7.59 (1 H, d, J=1.59 Hz), 7.76 (1 H, dd, J=8.68, 1.47 Hz), 7.99 (1 H, d, J=8.80 Hz), 8.02 (1 H, s), 9.09 (1 H, s), 11.56 (1 H, br s); ESIMS found for C₂₀1-1₂₃N₇O m/z 378.3 (M+1).

### Example 15

Representative compounds were screened using the assay procedure for DYRK1A kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, CA) into 1536-well black-walled round bottom plates (Corning).

The DYRK1A kinase assay was run using the Ser/Thr 18 peptide Z-lyte assay kit according to manufacturer's instructions (Life Technologies-a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant DYRK1A kinase, ATP and Ser/Thr peptide 18 were prepared in 1X Kinase buffer to final concentrations of 0.25 μg/mL, 15 μM, and 4μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (luM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio X F100%)−C100%)/((C0%-C100%)+(Em ratio X (F100%−F0%)))]. Dose-response curves were generated, and inhibitory concentration (IC50) values were calculated using non-linear regression curve fit in the Dotmatics' Studies Software (Bishops Stortford, UK).

Table 2 shows the measured activity for representative compounds of

Formulas I, Ia, Ib, and Ic, as described herein.

TABLE 2

| Compound | EC₅₀ (μM) |
|---|---|
| 1 | 0.2358 |
| 2 | 0.0438 |
| 3 | 0.0221 |
| 4 | 0.4107 |
| 5 | 1.4035 |
| 6 | 0.0339 |
| 7 | >10 |
| 8 | 0.7681 |
| 9 | 0.7777 |
| 10 | 0.0968 |
| 24 | 0.0145 |
| 54 | 0.6713 |
| 87 | 0.0068 |
| 93 | 0.0064 |
| 98 | 0.0221 |
| 103 | 0.0219 |
| 107 | 0.0016 |
| 110 | 0.0360 |
| 112 | 0.0028 |
| 114 | 0.0046 |
| 115 | 0.0031 |
| 116 | 0.0096 |
| 145 | 0.0943 |
| 154 | 0.0259 |
| 155 | 0.0117 |
| 158 | 0.0055 |
| 161 | 0.0265 |
| 187 | 0.0203 |
| 189 | 0.0242 |
| 196 | 0.0605 |
| 197 | 0.0754 |
| 198 | 0.0195 |
| 199 | 0.0155 |
| 202 | 0.0060 |
| 204 | 0.0066 |
| 205 | 0.0052 |
| 207 | 0.0274 |
| 208 | 0.0071 |
| 216 | 0.0149 |
| 219 | 0.0055 |
| 221 | 0.0230 |
| 233 | 0.2684 |
| 258 | 0.0453 |
| 276 | 0.0284 |
| 285 | 0.0127 |
| 292 | 0.0170 |
| 293 | 0.0936 |
| 301 | 0.0066 |
| 329 | 0.0068 |
| 359 | 0.1146 |
| 363 | 0.0416 |
| 403 | 0.1742 |
| 407 | 0.0012 |
| 448 | 0.0093 |
| 449 | 0.0094 |
| 450 | 0.0161 |
| 452 | 0.0202 |
| 460 | 0.0015 |
| 464 | 0.0095 |
| 469 | 0.0076 |
| 490 | 0.1526 |
| 491 | 0.0155 |
| 492 | 0.1229 |
| 493 | 4.7047 |
| 494 | 0.2271 |
| 497 | 0.0092 |
| 499 | 0.0060 |
| 500 | 0.0198 |
| 506 | 0.0017 |
| 507 | 0.0072 |
| 526 | 0.0085 |
| 529 | 0.0093 |
| 539 | 0.0408 |
| 540 | 0.0255 |
| 544 | 0.0122 |
| 585 | 0.0179 |
| 588 | 0.0250 |
| 594 | 0.0056 |
| 627 | 0.0713 |
| 628 | 0.7887 |
| 629 | 0.5306 |
| 643 | 0.0428 |
| 646 | 0.0056 |
| 674 | 0.0051 |
| 676 | 0.0015 |
| 718 | 0.4147 |
| 720 | 0.1998 |
| 721 | 0.8523 |
| 726 | 0.0224 |
| 727 | 0.0069 |
| 728 | 0.0481 |
| 730 | 0.0336 |
| 731 | 0.0086 |
| 732 | 0.0117 |
| 733 | 0.0230 |
| 734 | 0.0187 |
| 735 | 0.0787 |
| 736 | 0.0458 |
| 739 | 0.0326 |
| 740 | 0.0220 |
| 743 | 0.0019 |
| 744 | 0.0065 |
| 745 | 0.0051 |
| 746 | 0.0292 |
| 748 | 0.0200 |
| 749 | 0.0070 |
| 750 | 0.0269 |
| 755 | 0.0045 |
| 771 | 0.0180 |
| 774 | 0.0763 |
| 804 | 0.0721 |
| 805 | 0.0113 |
| 806 | 0.0188 |
| 807 | 0.1652 |
| 808 | 0.0523 |
| 810 | 0.0660 |
| 811 | 0.0333 |
| 822 | 0.0029 |
| 823 | 0.0060 |
| 824 | 0.0034 |
| 825 | 0.0048 |
| 826 | 0.0430 |
| 831 | 0.0073 |
| 838 | 0.0098 |
| 841 | 0.0136 |
| 843 | 0.0232 |
| 865 | 0.0073 |
| 895 | 0.0832 |
| 897 | 0.0668 |
| 907 | 0.0088 |
| 909 | 0.0034 |
| 910 | 0.0186 |
| 911 | 0.0096 |
| 912 | 0.0055 |
| 913 | 0.0064 |
| 918 | 0.0034 |
| 919 | 0.0040 |
| 920 | 0.0061 |
| 923 | 0.0025 |
| 924 | 0.0032 |
| 925 | 0.0032 |
| 930 | 0.0040 |
| 952 | 0.0027 |
| 991 | 0.0257 |
| 995 | 0.0093 |
| 1038 | 0.0042 |
| 1057 | 0.0098 |
| 1081 | 0.0026 |
| 1113 | 0.0092 |
| 1115 | 0.0249 |
| 1125 | 0.0046 |
| 1128 | 0.0024 |
| 1140 | 0.0026 |
| 1141 | 0.3846 |
| 1142 | 0.0057 |
| 1143 | 0.0037 |
| 1146 | 0.0285 |
| 1148 | 0.0042 |
| 1157 | 0.0689 |
| 1190 | 0.0030 |
| 1205 | 0.2589 |
| 1214 | 0.0097 |
| 1233 | 0.0181 |
| 1244 | 0.4943 |
| 1245 | 0.1822 |
| 1249 | 0.0016 |
| 1250 | 0.0146 |
| 1292 | 0.0023 |
| 1335 | 0.0177 |
| 1378 | 0.0026 |
| 1379 | 0.0798 |
| 1422 | 0.0060 |
| 1428 | 0.0114 |
| 1465 | 0.0067 |
| 1509 | 0.0399 |
| 1511 | 0.0034 |
| 1516 | 0.0058 |
| 1535 | 0.0012 |
| 1551 | 0.0075 |
| 1552 | 0.1945 |
| 1624 | 0.0037 |
| 1636 | 0.2136 |
| 1646 | 0.0191 |
| 1648 | 0.0039 |
| 1649 | 0.0059 |
| 1650 | 0.0198 |
| 1651 | 0.0132 |
| 1652 | 0.1030 |
| 1658 | 0.0105 |
| 1659 | 0.0199 |
| 1662 | 0.0134 |
| 1663 | 0.0092 |
| 1664 | 0.0060 |
| 1667 | 0.0190 |
| 1669 | 0.0061 |
| 1678 | 0.0211 |
| 1679 | 0.0929 |
| 1680 | 0.0261 |
| 1681 | 0.0988 |
| 1695 | 0.0051 |
| 1758 | 0.0073 |
| 1768 | 0.0348 |
| 1801 | 0.0669 |
| 1849 | 0.0021 |
| 1857 | 0.0427 |
| 1862 | 0.0029 |
| 1863 | 0.0029 |
| 1867 | 0.0697 |
| 1869 | 0.0040 |
| 1885 | 0.0090 |
| 1888 | 0.0031 |
| 1890 | 0.0060 |
| 1891 | 0.0041 |
| 1892 | 0.0008 |
| 1893 | 0.0005 |
| 1894 | 0.0023 |
| 1895 | 0.0036 |
| 1896 | 0.0156 |
| 1897 | 0.0181 |
| 1898 | 0.0075 |
| 1899 | 0.0186 |
| 1911 | 0.1171 |
| 1913 | 0.0049 |
| 1916 | 0.0060 |
| 1928 | 0.0037 |
| 1929 | 0.0053 |
| 1932 | 0.0125 |
| 1934 | 0.0078 |
| 1938 | 0.0033 |
| 1956 | 0.0119 |
| 1982 | 0.0029 |
| 1986 | 0.0416 |
| 1987 | 0.0704 |
| 1994 | 0.0138 |
| 2031 | 0.0128 |
| 2032 | 0.0588 |
| 2051 | 0.0078 |
| 2053 | 0.0019 |
| 2070 | 0.0029 |
| 2075 | 0.0080 |
| 2076 | 0.0057 |
| 2104 | 0.6900 |
| 2129 | 0.0024 |
| 2143 | 0.0099 |
| 2145 | 0.0030 |
| 2162 | 0.0085 |
| 2171 | 0.0015 |
| 2172 | 0.0050 |
| 2173 | 0.0105 |
| 2174 | 0.0016 |
| 2175 | 0.0749 |
| 2176 | 0.0021 |
| 2177 | 0.0103 |
| 2178 | 0.0068 |
| 2179 | 0.0022 |
| 2180 | 0.0039 |
| 2181 | 0.0051 |
| 2182 | 0.0031 |
| 2183 | 0.0104 |
| 2184 | 0.0699 |
| 2185 | 0.0820 |
| 2186 | 0.0023 |
| 2187 | 0.0036 |
| 2188 | 0.0019 |
| 2189 | 0.0081 |
| 2190 | 0.0030 |
| 2191 | 0.0047 |
| 2192 | 0.0069 |
| 2193 | 0.0021 |
| 2194 | 0.0055 |
| 2195 | 0.0094 |
| 2196 | 0.0116 |
| 2197 | 0.0015 |
| 2198 | 0.1696 |
| 2199 | 0.0022 |
| 2200 | 0.0026 |
| 2201 | 0.0050 |
| 2202 | 0.0039 |
| 2203 | 0.0034 |
| 2204 | 0.0154 |
| 2205 | 0.0795 |
| 2206 | 0.0032 |
| 2207 | 0.0017 |
| 2208 | 0.0043 |
| 2209 | 0.0016 |
| 2210 | 0.0034 |
| 2211 | 0.1260 |
| 2212 | 0.0066 |
| 2213 | 0.0719 |
| 2214 | 0.0017 |
| 2215 | 0.0016 |
| 2216 | 0.0074 |
| 2217 | 0.0022 |
| 2218 | 0.0129 |
| 2219 | 0.0035 |
| 2220 | 0.0661 |
| 2221 | 0.0036 |
| 2222 | 0.0058 |
| 2223 | 0.0094 |
| 2224 | 0.0107 |
| 2225 | 0.0154 |
| 2226 | 0.0062 |
| 2227 | 0.1752 |
| 2228 | 0.0026 |
| 2229 | 0.0026 |
| 2230 | 0.0045 |
| 2231 | 0.0022 |
| 2232 | 0.0013 |
| 2233 | 0.0016 |
| 2234 | 0.0059 |
| 2235 | 0.0074 |
| 2236 | 0.0150 |
| 2237 | 0.0079 |
| 2238 | 0.0352 |
| 2239 | 0.0069 |
| 2240 | 0.0079 |
| 2241 | 0.0269 |
| 2242 | 0.0284 |
| 2245 | 0.0296 |
| 2246 | 0.1325 |
| 2254 | 0.0258 |
| 2255 | 0.0190 |
| 2256 | 0.0150 |
| 2258 | 0.1933 |
| 2260 | 0.2452 |
| 2268 | 0.0099 |
| 2269 | 0.0772 |
| 2271 | 0.1124 |
| 2272 | 0.0622 |
| 2276 | 0.2562 |
| 2278 | 1.0023 |
| 2279 | 0.2500 |
| 2281 | 0.2693 |
| 2283 | 0.0474 |
| 2284 | 0.0839 |
| 2285 | 0.2003 |
| 2287 | 0.2391 |
| 2289 | 1.4823 |
| 2292 | 0.0174 |
| 2294 | 0.0237 |
| 2296 | 0.1032 |
| 2297 | 0.4585 |
| 2298 | 0.1167 |
| 2300 | 0.2300 |
| 2305 | 0.0218 |
| 2306 | 0.1706 |
| 2308 | 0.4616 |
| 2314 | 0.0173 |
| 2315 | 0.1196 |
| 2316 | 0.1595 |
| 2317 | 0.1067 |
| 2318 | 0.0129 |
| 2319 | 0.2869 |
| 2320 | 0.0594 |
| 2323 | 0.0009 |
| 2324 | 0.0015 |
| 2325 | 0.0870 |
| 2349 | 0.0013 |
| 2378 | 0.0011 |
| 2494 | 0.0011 |
| 2817 | 5.4387 |
| 3018 | 0.0044 |
| 3047 | 0.0012 |
| 3053 | 0.0014 |
| 3114 | 0.0012 |
| 3115 | 0.0013 |
| 3119 | 0.0002 |
| 3120 | 0.0042 |
| 3127 | 0.0014 |
| 3128 | 0.0006 |
| 3133 | 0.0011 |
| 3224 | 0.0026 |
| 3277 | 0.0019 |
| 3278 | 0.0192 |
| 3291 | 0.0011 |
| 3310 | 0.2297 |
| 3311 | 0.0014 |
| 3321 | 0.0375 |
| 3326 | 0.0016 |
| 3384 | 0.0012 |
| 3420 | 0.0056 |
| 3421 | 0.0281 |
| 3426 | 0.0032 |
| 3435 | 0.0023 |
| 3454 | 0.0034 |
| 3459 | 0.2095 |
| 3461 | 0.0019 |
| 3575 | 0.0007 |
| 3590 | 0.0016 |
| 3643 | 0.0007 |

### Example 16

Representative compounds were screened using the assay procedure for tau phosphorylation activity described below.

HEK293T cells (ATCC, CRL3216) cultured in DMEM (Thermo Fisher Scientific, 10566024) supplemented with 10% FBS (Corning, 35-011—CV) and Penicillin/Streptomycin (Thermo Fisher Scientific, 15140163) were seeded in a 75 cm² flask at 8.1 x 10⁶ cells/flask. The HEK293T cells were then transiently transfected with 5μg DYRK1A (NM_001396) human untagged clone (OriGene, SC314641) and 2.5 ₗag MAPT (441 a.a. Tau gene) (NM_005910) human untagged clone (OriGene, TP313312) using Lipofectamine 3000 (Thermo Fisher Scientific, L30000015) and incubated for 20-30 hours in a humidified incubator at 37° C. and 5% CO₂. Post-incubation, HEK293T cells transfected with the DYRK1A and MAPT expression vectors were harvested and seeded in BioCoat poly-D lysine coated 96-well plates (Corning, 354461) at 3×10⁴ cells/well.

The above synthesized compounds were screened using the cell assay procedure to assess decreased Tau phosphorylation at Thr212 (pThr212) described below.

Each compound was dissolved in DMSO (Sigma-Aldrich, D8418-100 mL) as a 10 mM stock. 10 mM stocks were serially diluted 1:3, 10-point dose-response curve and added to the cells with a final concentration ranging from 20 uM to 1.1 nM. Cell were treated with compounds in duplicate and incubated for 18-24 hours in a humidified incubator at 37° C. and 5% CO₂.

Following the overnight compound treatment, cells were lysed with 1X Alpha Surefire Ultra Lysis Buffer (Perkin Elmer, ALSU-LB-100ML) complemented with 1X Halt Phosphatase Inhibitor Cocktail (Thermo Fisher Scientific, 78427) and 1X Halt Protease Inhibitor Cocktail (Thermo Fisher Scientific, 78438). Lysates were spun down at 12,000g for 10 min to remove any cellular debris and 5μL of lysates were dispensed into a 384-well Opti-Plate (Perkin Elmer, 6007290) for the measurement of Tau phosphorylation in the phosphoTau (Thr212) AlphaLISA assay. Donor antibody, biotinylated HT7Tau (Thermo Fisher Scientific, MN1000B), and acceptor antibody, pThr212Tau (Thermo Fisher Scientific, 44740G) were both added to the cell lysates at a final concentration of 3 nM and incubated for 1 hour at room temperature. Following incubation of the lysates with the donor and acceptor antibodies, anti-rabbit IgG(Fc specific) AlphaLISA acceptor beads (Perkin Elmer, AL104C) were added at a 10 ug/mL final concentration and incubated for 1 hour at room temperature protected from light. Lastly, AlphaScreen streptavidin donor beads (PerkinElmer, 6760002) were added at 40 ug/mL final concentration and incubated for 1 hour at room temperature protected from light. Plates were read at Ex=665 nm, and Em=615 nm on the EnVision Multilabel Plate Reader (Perkin Elmer). Phospho-Tau (Thr212) AlphaLISA signal was used to plot, draw the curve fitting, and determine each compound's EC50 in Prism (GraphPad).

Table 3 shows the activity of representative compounds of Formulas I, Ia, Ib, and Ic, as described herein.

TABLE 3

| | pTau (Thr212) |
|---|---|
| Compound | EC₅₀ (μM) |
| 24 | 0.360 |
| 87 | 0.107 |
| 103 | 4.499 |
| 110 | 0.022 |
| 112 | 0.113 |
| 115 | 0.030 |
| 116 | 0.117 |
| 154 | 0.476 |
| 155 | 0.227 |
| 161 | 0.361 |
| 187 | 0.765 |
| 189 | 1.664 |
| 198 | 0.351 |
| 199 | 0.049 |
| 202 | 0.076 |
| 205 | 0.124 |
| 208 | 0.030 |
| 276 | 0.657 |
| 285 | 0.241 |
| 292 | 0.509 |
| 301 | 0.066 |
| 329 | 0.211 |
| 407 | 0.011 |
| 448 | 0.245 |
| 449 | 0.497 |
| 450 | 0.980 |
| 452 | 1.221 |
| 460 | 0.057 |
| 469 | 0.246 |
| 497 | 0.433 |
| 499 | 0.296 |
| 500 | 0.338 |
| 507 | 0.037 |
| 544 | 0.408 |
| 588 | 1.470 |
| 629 | 0.028 |
| 646 | 0.006 |
| 674 | 0.072 |
| 726 | 0.815 |
| 727 | 0.078 |
| 730 | 0.504 |
| 731 | 0.199 |
| 732 | 0.191 |
| 734 | 0.335 |
| 739 | 0.265 |
| 740 | 0.226 |
| 744 | 0.213 |
| 745 | 0.090 |
| 746 | 0.521 |
| 748 | 0.174 |
| 749 | 0.080 |
| 750 | 0.067 |
| 771 | 0.470 |
| 805 | 0.439 |
| 831 | 0.165 |
| 838 | 0.025 |
| 909 | 0.040 |
| 910 | 0.283 |
| 912 | 0.096 |
| 913 | 0.228 |
| 919 | 0.030 |
| 920 | 0.070 |
| 923 | 0.020 |
| 924 | 0.020 |
| 925 | 0.025 |
| 1038 | 0.033 |
| 1113 | 0.572 |
| 1115 | 0.386 |
| 1140 | 0.006 |
| 1141 | 0.007 |
| 1142 | 0.032 |
| 1143 | 0.004 |
| 1148 | 0.011 |
| 1214 | 0.219 |
| 1233 | 0.116 |
| 1249 | 0.027 |
| 1250 | 0.141 |
| 1292 | 0.054 |
| 1335 | 0.236 |
| 1378 | 0.604 |
| 1509 | 7.018 |
| 1516 | 0.034 |
| 1535 | 0.024 |
| 1624 | 3.908 |
| 1648 | 0.152 |
| 1658 | 0.120 |
| 1659 | 0.141 |
| 1662 | 0.103 |
| 1664 | 0.056 |
| 1849 | 0.168 |
| 1885 | 0.002 |
| 1888 | 0.022 |
| 1913 | 0.047 |
| 1928 | 0.111 |
| 1929 | 0.010 |

What is claimed is:

1. A compound, or a pharmaceutically acceptable salt thereof, of Formula I: 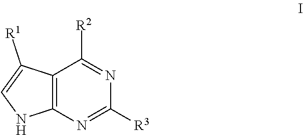
wherein:
R¹ is 8-11-membered bicyclic heteroaryl optionally substituted with 1-10 R⁴;
wherein a carbon atom on an aromatic ring of the heteroaryl forms the bond with 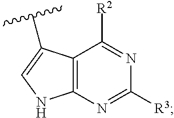
R² is selected from the group consisting of H, —OR⁷, 5-membered heteroaryl optionally substituted with 1-3 R⁸, and —NHR⁹;
R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;
with the proviso that R² and R³ are not both H;
each R⁴ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;
alternatively, two R⁴ attached to the same carbon atom are taken together to form a carbonyl group;
R⁷ is selected from the group consisting of unsubstituted —(C₁₋₉ alkyl), unsubstituted —(C₁₋₂-alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;
each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R⁹ is selected from the group consisting of unsubstituted —(C₁₋₉ alkyl), unsubstituted —(C₂₋₉-alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;
R¹⁰ is selected from the group consisting of unsubstituted —(C₁₋₉ alkyl), unsubstituted —(C₂₋₉-alkenyl), unsubstituted —(C₂₋₉ alkynyl), and unsubstituted —(C₁₋₉ haloalkyl);
each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;
each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹⁴ is selected from the group consisting of unsubstituted —(C₁₋₉ alkyl), unsubstituted —(C₂₋₉alkenyl), unsubstituted —(C₂₋₉ alkynyl), unsubstituted —(C₁₋₉ haloalkyl), —(C₁₋₅ alkylene)OMe, —(C₁₋₃ alkylene)pcarbocyclyl optionally substituted with 1-12 R¹', —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);
each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹' is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;
each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;
each R²⁰ is -heterocyclyl optionally substituted with 1-10 R²⁸;
each R²¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);
each R²² is selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R²⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R²⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R²⁶ is independently selected from the group consisting of halide, —OR³², —C(═O)R³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;
alternatively, two R²⁶ attached to the same carbon atom are taken together to form a carbonyl group;
each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C1-5 alkylene)OR³²;
each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);
each R³⁴ is independently selected from the group consisting of —N(R³⁷)₂, and -heterocyclyl optionally substituted with 1-10 R²⁸;
each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
alternatively, two R³⁵ attached to the same carbon atom are taken together to form a carbonyl group;
each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1;
wherein each H atom is optionally independently replaced by ²H (D) (deuterium); and
with the proviso that Formula I is not a structure selected from the group consisting of:

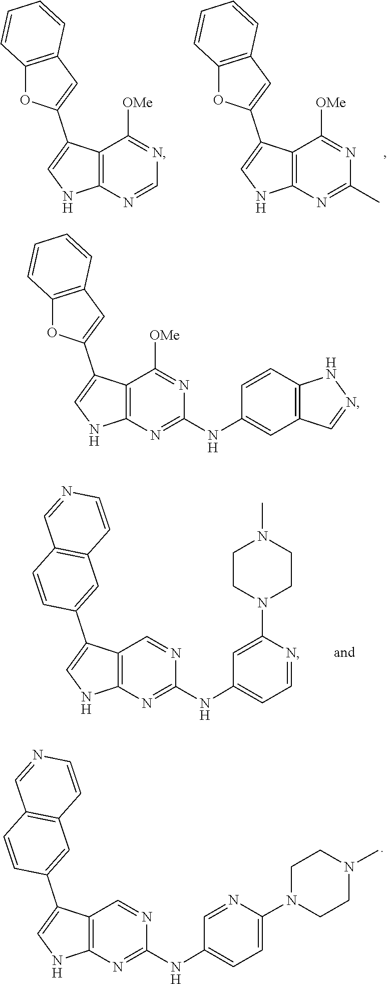

2. The compound of claim 1, wherein R² is selected from the group consisting of H, —OR⁷, and —NHR⁹.

3. The compound of claim 2, wherein R⁷ is selected from the group consisting of unsubstituted —(C₁₋₃ alkyl) and unsubstituted —(C₁₋₃haloalkyl).

4. The compound of claim 2, wherein R⁹ is selected from the group consisting of unsubstituted —(C₁₋₃ alkyl) and unsubstituted —(C₁₋₃haloalkyl).

5. The compound of claim 2, wherein R¹ is 9-10-membered bicyclic heteroaryl optionally substituted with 1-2 R⁴.

6. The compound of claim 5, wherein R¹ is 9-10-membered bicyclic heteroaryl optionally substituted with 1-2 R⁴, wherein 1-2 R⁴ is selected from the group consisting of halide, unsubstituted —(C₁₋₃ alkyl), or unsubstituted —(C₁₋₃ haloalkyl).

7. The compound of claim 6, wherein R¹ is selected from the group consisting of: 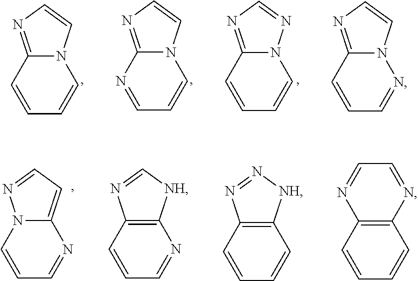
optionally substituted with 1-2 R⁴.

8. The compound of claim 7, wherein R¹ selected from the group consisting of: 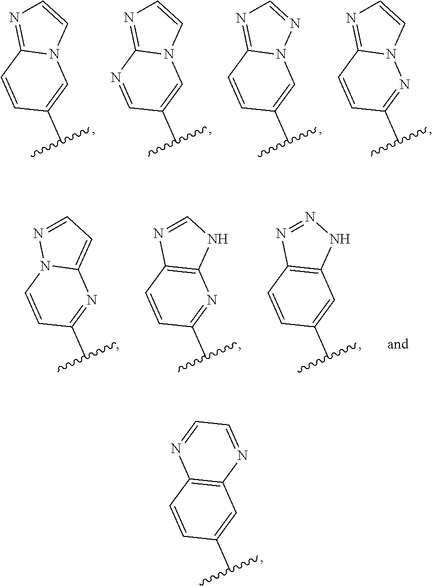
optionally
substituted with 1-2 R⁴.

9. The compound of claim 8, wherein R¹ selected from the group consisting of:

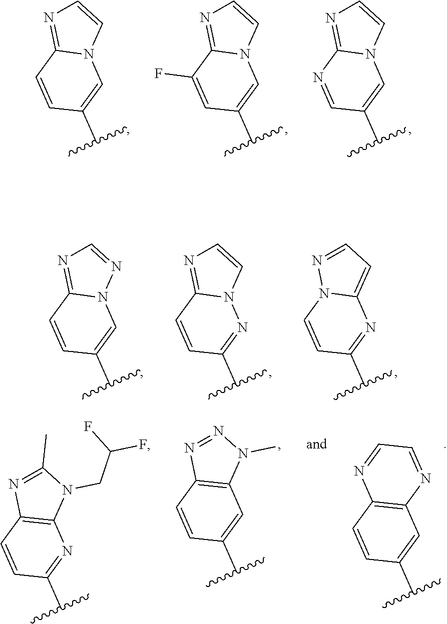

10. The compound of claim 9, wherein R³ is selected from the group consisting of -carbocyclyl optionally substituted with 1-2 R¹³ and —NHR¹⁴.

11. The compound of claim 10, wherein R^{H} is selected from the group consisting of -carbocyclyl optionally substituted with 1-5 R¹⁷ and -heterocyclyl optionally substituted with 1-5 R²⁶.

12. A compound, or a pharmaceutically acceptable salt thereof, of Formula Ic: 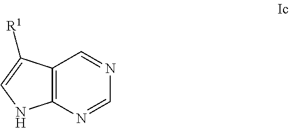
wherein:
R¹ is selected from the heteroaryl group consisting of: 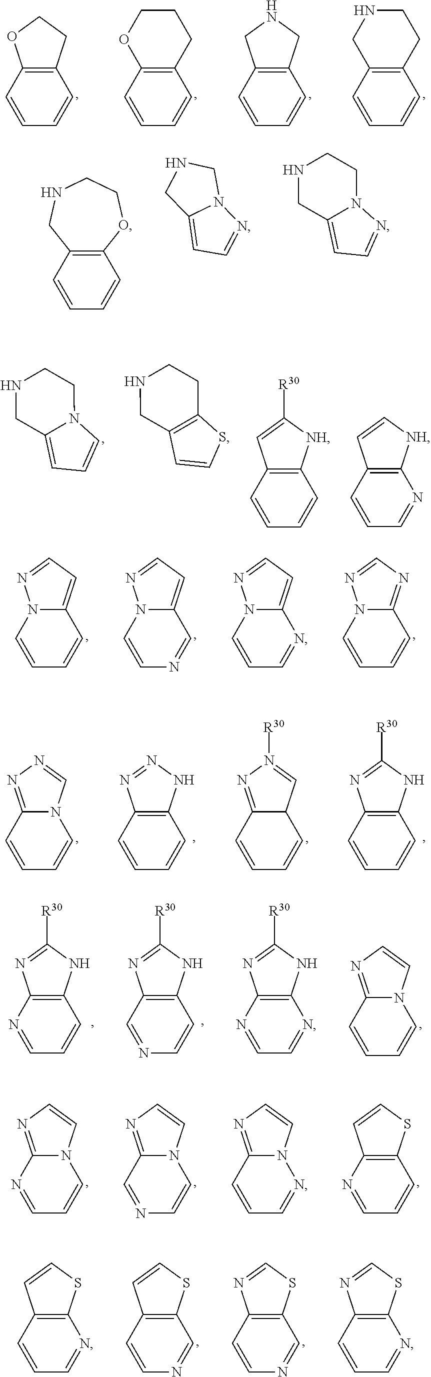 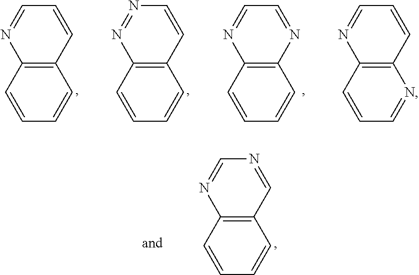
optionally substituted with 1-10 R⁴;
wherein a carbon atom on an aromatic ring of the heteroaryl forms the bond with 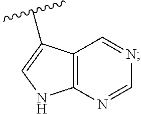 [SMILES: *c1c[nH]c2ncncc12]
each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R¹¹, —C(═O)N(R¹⁹)2, and —C(═O)R²⁰;
alternatively, two R⁴ attached to the same carbon atom are taken together to form a carbonyl group;
each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;
each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;
each R²⁰ is -heterocyclyl optionally substituted with 1-10 R²⁸;
each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁸ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);
each R²⁹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);
each R³° is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;
each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁴ is independently selected from the group consisting of —N(R³⁷)2, and -heterocyclyl optionally substituted with 1-10 R²⁸;
each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
alternatively, two R³⁵ attached to the same carbon atom are taken together to form a carbonyl group;
each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1; and
wherein each H atom is optionally independently replaced by ²H (D) (deuterium).

13. The compound of claim 12, wherein R¹ is selected from the group consisting of: 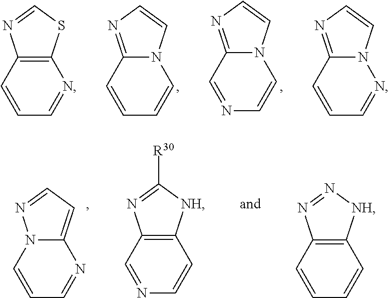
optionally
substituted with 1-2 R⁴.

14. The compound of claim 13, wherein R¹ is selected from the group consisting of: 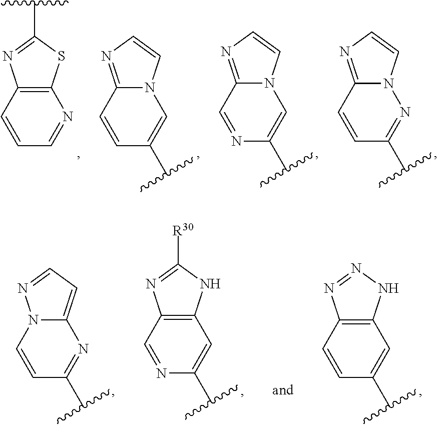
optionally
substituted with 1-2 R⁴.

15. A compound, or a pharmaceutically acceptable salt thereof, of Formula I: 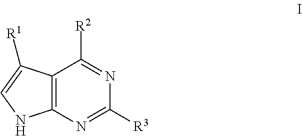
wherein:
R¹ is selected from the group consisting of phenyl substituted with 1-5 R⁵, 6-membered heteroaryl optionally substituted with 1-4 R⁶, and 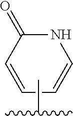
optionally substituted with 1-4 R⁶;
R² is selected from the group consisting of H, 5-membered heteroaryl optionally substituted with 1-3 R⁸, and —NHR⁹,
R³ is selected from the group consisting of H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹¹, —(5-10 membered heteroaryl) optionally substituted with 1-10 R¹², —(CH₂)ₚcarbocyclyl optionally substituted with 1-12 R¹³, and —NHR¹⁴;
with the proviso that either R² or R³ is H but R² and R³ are not both H;
each R⁵ is independently selected from the group consisting of halide, unsubstituted —(C₁₋₉ alkyl), unsubstituted —(C₂₋₉ alkenyl), unsubstituted —(C₂₋₉ alkenyl), unsubstituted —(C₁₋₉ haloalkyl),
—OR¹⁵, —CH₂OH, -heterocyclyl optionally substituted with 1-10 R¹⁶, -heteroaryl optionally substituted with 1-10 R¹⁷, —carbocyclyl optionally substituted with 1-12 R⁸, —C(═O)N(R¹⁹)2, and —C(═O)R²⁰;
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR^{15a}, —CH₂OH, -heterocyclyl optionally substituted with 1-10 R^{16a}, -heteroaryl optionally substituted with 1-10 R^{17a}, —carbocyclyl optionally substituted with 1-12 R^{18a}, —C(═O)N(R¹⁹a)₂, and —C(═O)R^{20a};
R⁷ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OR³², and —(CH₂)heterocyclyl optionally substituted with 1-10 R²¹;
each R⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R⁹ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚcarbocyclyl optionally substituted with 1-12 R²², and -heterocyclyl optionally substituted with 1-10 R²³;
R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C1-5 alkylene)OR³²;
each R¹² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹³ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹⁴ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₅ alkylene)OMe, —(C₁₋₃ alkylene)ₚcarbocyclyl optionally substituted with 1-12 R¹⁷, —(C₁₋₃ alkylene)ₚheteroaryl optionally substituted with 1-10 R²⁴, —(C₁₋₃ alkylene)ₚphenyl optionally substituted with 1-10 R²⁵, and —(C₁₋₃ alkylene)ₚheterocyclyl optionally substituted with 1-10 R²⁶, wherein each —(C₁₋₃ alkylene) is, independently, optionally substituted with 1-5 halide and/or 1-5 unsubstituted —(C₁₋₃ alkyl);
each R¹⁵ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R^{15a} is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R^{16a};
each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R^{16a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁷ is independently selected from the group consisting of halide, —OR³¹, —NHC(═O)R³³, —C(═O)R³⁴, unsubstituted —(C₁₋₉ alkyl), unsubstituted —(C₂₋₉ alkenyl), unsubstituted —(C₂₋₉ alkynyl), and unsubstituted —(C₁₋₉ haloalkyl), and -heterocyclyl optionally substituted with 1-10 R³⁵;
each R^{17a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl) ;
each R¹⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R^{18a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁹ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R²⁷, -heterocyclyl optionally substituted with 1-10 R²⁸, and -heteroaryl optionally substituted with 1-10 R²⁹;
each R^{19a} are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚcarbocyclyl optionally substituted with 1-12 R^{27a}, -heterocyclyl optionally substituted with 1-10 R^{28a}, and -heteroaryl optionally substituted with 1-10 R^{29a};
each R²⁰ is -heterocyclyl optionally substituted with 1-10 R²⁸;
each R^{26a} is -heterocyclyl optionally substituted with 1-10 R^{28a};
each R²¹ is independently selected from the group consisting of halide, unsubstituted —(C alkyl), unsubstituted —(C₂₋₉ alkenyl), unsubstituted —(C₂₋₉ alkynyl), and unsubstituted —(C₁₋₉ haloalkyl);
each R²² is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²³ is independently selected from the group consisting of halide, —OMe, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R²⁴ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R²⁵ is independently selected from the group consisting of halide, unsubstituted —(C₁₋₉ 9alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R¹⁶;
each R²⁶ is independently selected from the group consisting of halide, —01e, —C(═O)12_³⁶, —SO₂Me, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R²⁸;
alternatively, two R²⁶ attached to the same carbon atom are taken together to form a carbonyl group;
each R²⁷ is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R^{27a} is independently selected from the group consisting of halide, —OMe, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R^{28a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R^{29a} is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³¹ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)OR³²;
each R³² is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁴ is independently selected from the group consisting of —N(R³⁷)2, and -heterocyclyl optionally substituted with 1-10 R²⁸;
each R³⁵ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
alternatively, two R³⁵ attached to the same carbon atom are taken together to form a carbonyl group;
each R³⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C ₁₋₉ haloalkyl);
each R³⁷ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1;
wherein each H atom is optionally independently replaced by ²H (D) (deuterium); and
with the proviso that Formula I is not a structure selected from the group consisting of:

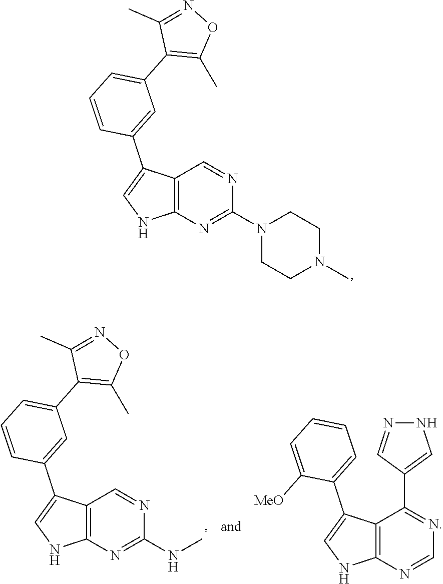 [SMILES: Cc1noc(C)c1-c1cccc(-c2c[nH]c3nc(N4CCN(C)CC4)ncc23)c1 ; CNc1ncc2c(-c3cccc(-c4c(C)noc4C)c3)c[nH]c2n1 ; COc1ccccc1-c1c[nH]c2ncnc(-c3cn[nH]c3)c12]

16. The compound of claim 15, wherein R² is selected from the group consisting of H, —OR⁷, and —NHR⁹.

17. The compound of claim 16, wherein R⁷ is selected from the group consisting of unsubstituted —(C₁₋₃ alkyl) and unsubstituted —(C₁₋₃haloalkyl).

18. The compound of claim 16, wherein R⁹ is selected from the group consisting of unsubstituted —(C₁₋₃ alkyl) and unsubstituted —(C₁₋₃haloalkyl).

19. The compound of claim 16, wherein R¹ is selected from the group consisting of 6-membered heteroaryl optionally substituted with 1-2 R⁶ and 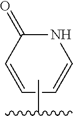
optionally substituted with 1-2 R⁶.

20. The compound of claim 19, wherein R¹ is selected from the group consisting of pyridine and 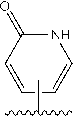
optionally substituted with one —OR^{15a} or one unsubstituted —(C₁₋₃ alkyl).

21. The compound of claim 20, wherein R¹ is selected from the group consisting of 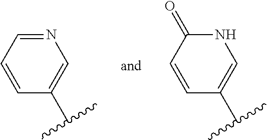 [SMILES: *c1cccnc1 ; *c1ccc(=O)[nH]c1]
optionally substituted with one —OR^{15a} or one unsubstituted —(C₁₋₃ alkyl).

22. The compound of claim 21, wherein R³ is selected from the group consisting of unsubstituted —(C1.5 alkyl) and —NHR¹⁴.

23. The compound of claim 1, wherein the compound of Formula I is selected from the group consisting of:
5-(2-(((6-(4-methylpiperazin-1-yl)pyridin-3-yl)methyl)amino)-7H-pyrrolo [2,3-d]pyrimidin-5-ypisoindolin-1-one [10];
2-methyl-6-(7H-pyrrolo [2,3-d] pyrimidin-5-yl)benzo [d]thiazole [11];
6-(2-isobutyl-7H-pyrrolo [2,3-d] pyrimidin-5-yl)-2-methylbenzo [d] thiazole [12];
2-methyl-6-(2-(3,3,3-trifluoropropyl)-7H-pyrrolo [2,3-d]pyrimidin-5-yl)benzo[d]thiazole [13];
6-(2-cyclopropyl-7H-pyrrolo [2,3-d]pyrimidin-5-yl)-2-methylbenzo[d]thiazole [14];
6-(2-cyclobutyl-7H-pyrrolo [2,3-d]pyrimidin-5-yl)-2-methylbenzo [d]thiazole [15];
6-(2-(cyclobutylmethyl)-7H-pyrrolo [2,3-d]pyrimidin-5-yl)-2-methylbenzo [d]thiazole [16];
5-(2-methylbenzo [d]thiazol-6-yl)-7H-pyrrolo [2,3-d] pyrimidin-2-amine [17];
N-methyl-5-(2-methylbenzo [d]thiazol -6-yl)-7H-pyrrolo [2,3-d] pyrimidin-2-amine [18];
N-ethyl-5-(2-methylbenzo [d]thiazol-6-yl)-7H-pyrrolo [2,3-d]pyrimidin-2-amine [19];
N-isopropyl-5-(2-methylbenzo [d]thiazol-6-yl)-7H-pyrrolo [2,3-d]pyrimidin-2-amine [20];
N-isobutyl-5-(2-methylbenzo [d]thiazol-6-yl)-7H-pyrrolo [2,3-d]pyrimidin-2-amine [21];
5-(2-methylbenzo [d]thiazol-6-yl)-N-ne opentyl-7H-pyrrolo [2,3-d]pyrimidin-2-amine [22];
(S)-N-(sec-butyl)-5-(2-methylbenzo [d]thiazol-6-yl)-7H-pyrrolo [2,3-d]pyrimidin-2-amine [23];
N-(2-fluoro-2-methylpropyl)-5-(2-methylbenzo [d]thiazol-6-yl)-7H-pyrrolo [2,3-d] pyrimidin-2-amine [24];
N-(2,2-difluoropropyl)-5-(2-methylbenzo [d]thiazol-6-yl)-7H-pyrrolo [2,3-d]pyrimidin-2-amine [25];
5-(2-methylbenzo [d]thiazol-6-yl)-N-(2,2,2-trifluoroethyl)-7H-pyrrolo [2,3-d]pyrimidin-2-amine [26];
(R)-5-(2-methylbenzo [d]thiazol-6-yl)-N-(1,1,1-trifluoropropan-2-yl)-7H-pyrrolo [2,3-d]pyrimidin-2-amine [27];
(S)-5-(2-methylbenzo [d]thiazol-6-yl)-N-(1,1,1-trifluoropropan-2-yl)-7H-pyrrolo [2,3-d]pyrimidin-2-amine [28];
5-(2-methylbenzo [d]thiazol-6-yl)-N-(3,3,3-trifluoropropyl)-7H-pyrrolo [2,3-d]pyrimidin-2-amine [29];
(S)-N-(1-methoxypropan-2-yl)-5-(2-methylbenzo [d]thiazol-6-yl)-7H-pyrrolo [2,3-d]pyrimidin-2-amine [30];
6-(2-ethoxy-7H-pyrrolo [2,3-d] pyrimidin-5-yl)-2-methylbenzo [d]thiazole [31];
N-((1-fluorocyclopropyl)methyl)-5-(2-methylbenzo [d]thiazol-6-yl)-7H-pyrrolo [2,3-d] pyrimidin-2-amine [32];
(R)-N-(1-cyclopropylethyl)-5-(2-methylbenzo [d]thiazol-6-yl)-7H-pyrrolo [2,3-d]pyrimidin-2-amine [33];
5-(2-methylbenzo [d]thiazol-6-yl)-N-((1-methylcyclopropyl)methyl)-7H-pyrrolo [2,3-d]pyrimidin-2-amine [34];
5-(2-methylbenzo [d]thiazol-6-yl)-N-41-(trifluoromethyl)cyclopropyl)methyl)-7H-pyrrolo [2,3-d]pyrimidin-2-amine [35];
N-(2-cyclopropyl-2,2-difluoroethyl)-5-(2-methylbenzo[d]thiazol-6-yl)-7H-pyrrolo[2,3-d]pyrimidin-2-amine [36];
N-(3,3-difluorocyclobutyl)-5-(2-methylbenzo[d]thiazol-6-yl)-7H-pyrrolo[2,3-d]pyrimidin-2-amine [37];
N-((1-fluorocyclobutyl)methyl)-5-(2-methylbenzo[d]thiazol-6-yl)-7H-pyrrolo[2,3-d]pyrimidin-2-amine [38];
N-(4,4-difluorocyclohexyl)-5-(2-methylbenzo[d]thiazol-6-yl)-7H-pyrrolo[2,3-d]pyrimidin-2-amine [39];
N-(cis-4-methoxycyclohexyl)-5-(2-methylbenzo[d]thiazol-6-yl)-7H-pyrrolo[2,3-d]pyrimidin-2-amine [40];
N-((1-fluorocyclohexyl)methyl)-5-(2-methylbenzo[d]thiazol-6-yl)-7H-pyrrolo[2,3-d]pyrimidin-2-amine [41];
N-((4,4-difluorocyclohexyl)methyl)-5-(2-methylbenzo[d]thiazol-6-yl)-7H-pyrrolo[2,3-d]pyrimidin-2-amine [42];
5-(2-methylbenzo [d]thiazol-6-yl)-N-(1-methylpiperidin-4-yl)-7H-pyrrolo [2,3-d]pyrimidin-2-amine [43];
5-(2-methylbenzo [d]thiazol-6-yl)-N-((1-methylpiperidin-4-yl)me thyl)-7H-pyrrolo [2,3-d]pyrimidin-2-amine [44];
5-(2-methylbenzo[d]thiazol-6-yl)-N-(tetrahydro-2H-pyran-4-yl)-7H-pyrrolo[2,3-d]pyrimidin-2-amine [45];
5-(2-methylbenzo[d]thiazol-6-yl)-N-(3-(4-methylpiperazin-1-yl)phenyl)-7H-pyrrolo[2,3-d]pyrimidin-2-amine [46];
5-(2-methylbenzo[d]thiazol-6-yl)-N-(pyridin-4-yl)-7H-pyrrolo[2,3-d]pyrimidin-2-amine [47];
5-(2-methylbenzo[d]thiazol-6-yl)-N-(2-(4-methylpiperazin-1-yl)pyridin-4-yl)-7H-pyrrolo[2,3-d]pyrimidin-2-amine [48];
5-(2-methylbenzo[d]thiazol-6-yl)-N-(6-(4-methylpiperazin-1-yl)pyridin-3-yl)-7H-pyrrolo[2,3-d]pyrimidin-2-amine [49];
5-(2-methylbenzo [d]thiazol-6-yl)-N-46-(4-methylpiperazin-1-yl)pyridin-3-yl)me thyl)-7H-pyrrolo[2,3-d]pyrimidin-2-amine [50];
2-methyl-6-(2-(quinolin-6-yl)-7H-pyrrolo[2,3-d]pyrimidin-5-yl)benzo[d]thiazole [51];
2-methyl-6-(4-(1-methyl-1H-pyrazol-4-yl)-7H-pyrrolo[2,3-d]pyrimidin-5-yl)benzo[d]thiazole [52];
5-(2-methylbenzo[d]thiazol-6-yl)-N-(trans-4-morpholinocyclohexyl)-7H-pyrrolo[2,3-d]pyrimidin-4-amine [53];
N-(2-fluoro-2-methylpropyl)-5-(2-methylthiazolo [5,4-b] pyridin-5-yl)-7H-pyrrolo [2,3-d]pyrimidin-2-amine [54];
2-(2-isobutyl-7H-pyrrolo [2,3-d] pyrimidin-5-yl)thieno [3,2-c]pyridine [56];
2-(2-(3,3,3-trifluoropropyl)-7H-pyrrolo [2,3-d]pyrimidin-5-yl)thieno [3,2-c]pyridine [57];
2-(2-cyclopropyl-7H-pyrrolo [2,3-d]pyrimidin-5-yl)thieno [3,2-c] pyridine [58];
2-(2-cyclobutyl-7H-pyrrolo [2,3-d]pyrimidin-5-yl)thieno [3,2-c]pyridine [59];
2-(2-(cyclobutylmethyl)-7H-pyrrolo [2,3-d]pyrimidin-5-yOthieno [3,2-c]pyridine [60];
5-(thieno [3,2-c]pyridin-2-yl)-7H-pyrrolo [2,3-d] pyrimidin-2-amine [61];
N-methyl-5-(thieno [3,2-c]pyridin-2-yl)-7H-pyrrolo [2,3-d] pyrimidin-2-amine [62];
N-ethyl-5-(thieno [3,2-c]pyridin-2-yl)-7H-pyrrolo [2,3-d]pyrimidin-2-amine [63];
N-isopropyl-5-(thieno [3,2-c]pyridin-2-yl)-7H-pyrrolo [2,3-d] pyrimidin-2-amine [64];
N-isobutyl-5-(thieno [3,2-c]pyridin-2-yl)-7H-pyrrolo [2,3-d]pyrimidin-2-amine [65];
N-neopentyl-5-(thieno [3,2-c] pyridin-2-yl)-7H-pyrrolo [2,3-d]pyrimidin-2-amine [66];
(S)-N-(sec-butyl)-5-(thieno [3,2-c]pyridin-2-yl)-7H-pyrrolo [2,3-d]pyrimidin-2-amine [67];
N-(2-fluoro-2-methylpropyl)-5-(thieno [3,2-c]pyridin-2-yl)-7H-pyrrolo [2,3-d]pyrimidin-2-amine [68];
N-(2,2-difluoropropyl)-5-(thieno [3,2-c] pyridin-2-yl)-7H-pyrrolo [2,3-d]pyrimidin-2-amine [69];
5-(thieno [3,2-c]pyridin-2-yl)-N-(2,2,2-trifluoroethyl)-7H-pyrrolo [2,3-d]pyrimidin-2-amine [70];
(R)-5-(thieno [3,2-c]pyridin-2-yl)-N-(1,1,1-trifluoropropan-2-yl)-7H-pyrrolo [2,3-d]pyrimidin-2-amine [71];
(S)-5-(thieno [3,2-c]pyridin-2-yl)-N-(1,1,1-trifluoropropan-2-yl)-7H-pyrrolo [2,3-d]pyrimidin-2-amine [72];
5-(thieno [3,2-c]pyridin-2-yl)-N-(3,3,3-trifluoropropyl)-7H-pyrrolo [2,3-d]pyrimidin-2-amine [73];
(S)-N-(1-methoxypropan-2-yl)-5-(thieno [3,2-c]pyridin-2-yl)-7H-pyrrolo [2,3-d]pyrimidin-2-amine [74];
2-(2-ethoxy-7H-pyrrolo [2,3-d] pyrimidin-5-yl)thieno [3,2-c]pyridine [75];
N-((1-fluorocyclopropyl)methyl)-5-(thieno [3,2-c]pyridin-2-yl)-7H-pyrrolo [2,3-d]pyrimidin-2-amine [76];
(R)-N-(1-cyclopropylethyl)-5-(thieno [3,2-c] pyridin-2-yl)-7H-pyrrolo [2,3-d]pyrimidin-2-amine [⁷⁷];
N-((1-methylcyclopropyl)methyl)-5-(thieno [3,2-c]pyridin-2-yl)-7H-pyrrolo [2,3-d]pyrimidin-2-amine [78];
5-(thieno [3,2-c]pyridin-2-yl)-N-41-(trifluoromethyl)cyclopropyl)methyl)-7H-pyrrolo [2,3-d]pyrimidin-2-amine [79];
N-(2-cyclopropyl-2,2-difluoroethyl)-5-(thieno [3,2-c]pyridin-2-yl)-7H-pyrrolo [2,3-d]pyrimidin-2-amine [80];
N-(3,3-difluorocyclobutyl)-5-(thieno [3,2-c]pyridin-2-yl)-7H-pyrrolo [2,3-d]pyrimidin-2-amine [81];
N-((1-fluorocyclobutyl)methyl)-5-(thieno [3,2-c] pyridin-2-yl)-7H-pyrrolo [2,3-d]pyrimidin-2-amine [82];
N-(4,4-difluorocyclohexyl)-5-(thieno [3,2-c]pyridin-2-yl)-7H-pyrrolo [2,3-d] pyrimidin-2-amine [83];
N-(cis-4-methoxycyclohexyl)-5-(thieno [3,2-c]pyridin-2-yl)-7H-pyrrolo [2,3-d]pyrimidin-2-amine [84];
N-((1-fluorocyclohexyl)methyl)-5-(thieno [3,2-c]pyridin-2-yl)-7H-pyrrolo [2,3-d]pyrimidin-2-amine [85];
N-((4,4-difluorocyclohexyl)methyl)-5-(thieno [3,2-c]pyridin-2-yl)-7H-pyrrolo [2,3-d]pyrimidin-2-amine [86];
N-(1-methylpiperidin-4-yl)-5-(thieno [3,2-c] pyridin-2-yl)-7H-pyrrolo [2,3-d]pyrimidin-2-amine [87];
N-((l-methylpiperidin-4-yl)methyl)-5-(thieno [3,2-c]pyridin-2-yl)-7H-pyrrolo [2,3-d] pyrimidin-2-amine [88];
N-(tetrahydro-2H-pyran-4-yl)-5-(thieno [3,2-c]pyridin-2-yl)-7H-pyrrolo [2,3-d]pyrimidin-2-amine [89];
N-(3-(4-methylpiperazin-1-yl)phenyl)-5-(thieno [3,2-c]pyridin-2-yl)-7H-pyrrolo [2,3-d] pyrimidin-2-amine [90];
N-(pyridin-4-yl)-5-(thieno [3,2-c]pyridin-2-yl)-7H-pyrrolo [2,3-d] pyrimidin-2-amine [91];
N-(2-(4-methylpiperazin-1-yl)pyridin-4-yl)-5-(thieno [3,2-c]pyridin-2-yl)-7H-pyrrolo [2,3-d]pyrimidin-2-amine [92];
N-(6-(4-methylpiperazin-1-yl)pyridin-3-yl)-5-(thieno [3,2-c]pyridin-2-yl)-7H-pyrrolo [2,3-d]pyrimidin-2-amine [93];
N-((6-(4-methylpiperazin-1-yl)pyridin-3-yl)methyl)-5-(thieno [3,2-c] pyridin-2-yl)-7H-pyrrolo [2,3-d]pyrimidin-2-amine [94];
2-(2-(quinolin-6-yl)-7H-pyrrolo [2,3-d] pyrimidin-5-yl)thieno [3,2-c] pyridine [95];
2-(4-(1-methyl-1H-pyrazol-4-yl)-7H-pyrrolo [2,3-d] pyrimidin-5-yl)thieno [3,2-c] pyridine [96];
N-(trans-4-morpholinocyclohexyl)-5-(thieno [3,2-c]pyridin-2-yl)-7H-pyrrolo [2,3-d]pyrimidin-4-amine [97];
5-(imidazo [1,2-a] pyridin-6-yl)-2-i sobutyl-7H-pyrrolo [2,3-d]pyrimidine [100];
5-(imidazo [1,2-a] pyridin-6-yl)-2-(3,3,3-trifluoropropyl)-7H-pyrrolo [2,3-d]pyrimidine [101];
2-cyclopropyl-5-(imidazo [1,2-a] pyridin-6-yl)-7H-pyrrolo [2,3-d]pyrimidine [102];
2-cyclobutyl-5-(imidazo [1,2-a] pyridin-6-yl)-7H-pyrrolo [2,3-d]pyrimidine [103];
2-(cyclobutylmethyl)-5-(imidazo [1,2-a] pyridin-6-yl)-7H-pyrrolo [2,3-d]pyrimidine [104];
5-(imidazo [1,2-a] pyridin-6-yl)-7H-pyrrolo [2,3-d]pyrimidin-2-amine [105];
5-(imidazo [1,2-a]pyridin-6-yl)-N-methyl-7H-pyrrolo [2,3-d]pyrimidin-2-amine [106];
N-ethyl-5-(imidazo [1,2-a] pyridin-6-yl)-7H-pyrrolo [2,3-d]pyrimidin-2-amine [107];
5-(imidazo [1,2-a]pyridin-6-yl)-N-isopropyl-7H-pyrrolo [2,3-d]pyrimidin-2-amine [108];
5-(imidazo [1,2-a] pyridin-6-yl)-N-isobutyl-7H-pyrrolo [2,3-d]pyrimidin-2-amine [109];
5-(imidazo [1,2-a] pyridin-6-yl)-N-neopentyl-7H-pyrrolo [2,3-d]pyrimidin-2-amine [110];
(S)-N-(sec-butyl)-5-(imidazo [1,2-a] pyridin-6-yl)-7H-pyrrolo [2,3-d] pyrimidin-2-amine [111];
N-(2-fluoro-2-methylpropyl)-5-(imidazo [1,2-a] pyridin-6-yl)-7H-pyrrolo [2,3-d] pyrimidin-2-amine [112];
N-(2,2-difluoropropyl)-5-(imidazo [1,2-a]pyridin-6-yl)-7H-pyrrolo [2,3-d]pyrimidin-2-amine
[113];
5-(imidazo [1,2-a]pyridin-6-yl)-N-(2,2,2-trifluoroethyl)-7H-pyrrolo [2,3-d] pyrimidin-2-amine
[114];
(R)-5-(imidazo [1,2-a]pyridin-6-yl)-N-(1,1,1-trifluoropropan-2-yl)-7H-pyrrolo [2,3-d]pyrimidin-2-amine [115];
(S)-5-(imidazo [1,2-a]pyridin-6-yl)-N-(1,1,1-trifluoropropan-2-yl)-7H-pyrrolo [2,3-d]pyrimidin-2-amine [116];
5-(imidazo [1,2-a]pyridin-6-yl)-N-(3,3,3-trifluoropropyl)-7H-pyrrolo [2,3-d] pyrimidin-2-amine
[117];
(S)-5-(imidazo [1,2-a] pyridin-6-yl)-N-(1-methoxypropan-2-yl)-7H-pyrrolo [2,3-d] pyrimidin-2-amine
[118];
2-ethoxy-5-(imidazo [1,2-a] pyridin-6-yl)-7H-pyrrolo [2,3-d]pyrimidine [119];
N-((1-fluorocyclopropyl)methyl)-5-(imidazo [1,2-a] pyridin-6-yl)-7H-pyrrolo [2,3-d]pyrimidin-2-amine
[120];
(R)-N-(1-cyclopropylethyl)-5-(imidazo [1,2-a] pyridin-6-yl)-7H-pyrrolo [2,3-d]pyrimidin-2-amine
[121];
5-(imidazo [1,2-a] pyridin-6-yl)-N-((1-methylcyclopropyl)methyl)-7H-pyrrolo [2,3-d]pyrimidin-2-amine [122];
5-(imidazo [1,2-a] pyridin-6-yl)-N-41-(trifluoromethyl)cyclopropyl)me thyl)-7H-pyrrolo [2,3-d]pyrimidin-2-amine [123];
N-(2-cyclopropyl-2,2-difluoroethyl)-5-(imidazo [1,2-a]pyridin-6-yl)-7H-pyrrolo [2,3-d]pyrimidin-2-amine [124];
N-(3,3-difluorocyclobutyl)-5-(imidazo [1,2-a] pyridin-6-yl)-7H-pyrrolo [2,3-d]pyrimidin-2-amine [125];
N-((1-fluorocyclobutyl)methyl)-5-(imidazo [1,2-a]pyridin-6-yl)-7H-pyrrolo [2,3-d] pyrimidin-2-amine [126];
N-(4,4-difluorocyclohexyl)-5-(imidazo [1,2-a]pyridin-6-yl)-7H-pyrrolo [2,3-d] pyrimidin-2-amine [127];
5-(imidazo [1,2-a] pyridin-6-yl)-N-(cis-4-methoxycyclohexyl)-7H-pyrrolo [2,3-d]pyrimidin-2-amine [128];
N-((1-fluorocyclohexyl)methyl)-5-(imidazo [1,2-a]pyridin-6-yl)-7H-pyrrolo [2,3-d]pyrimidin-2-amine [129];
N-((4,4-difluorocyclohexyl)methyl)-5-(imidazo [1,2-a]pyridin-6-yl)-7H-pyrrolo [2,3-d]pyrimidin-2-amine [130];
5-(imidazo [1,2-a] pyridin-6-yl)-N-(1-methylpipe ridin-4-yl)-7H-pyrrolo [2,3-d]pyrimidin-2-amine
[131];
5-(imidazo [1,2-a] pyridin-6-yl)-N-((1-methylpipe ridin-4-yl)methyl)-7H-pyrrolo [2,3-d]pyrimidin-2-amine [132];
5-(imidazo [1,2-a] pyridin-6-yl)-N-(tetrahydro-2H-pyran-4-yl)-7H-pyrrolo [2,3-d] pyrimidin-2-amine [133];
5-(imidazo [1,2-a] pyridin-6-yl)-N-(3-(4-methylpiperazin-1-yl)phenyl)-7H-pyrrolo [2,3-d]pyrimidin-2-amine [134];
5-(imidazo [1,2-a] pyridin-6-yl)-N-(pyridin-4-yl)-7H-pyrrolo [2,3-d] pyrimidin-2-amine [135];
5-(imidazo [1,2-a] pyridin-6-yl)-N-(2-(4-methylpiperazin-1-yl)pyridin-4-yl)-7H-pyrrolo [2,3-d]pyrimidin-2-amine [136];
5-(imidazo [1,2-a] pyridin-6-yl)-N-(6-(4-methylpiperazin-1-yl)pyridin-3-yl)-7H-pyrrolo [2,3-d]pyrimidin-2-amine [137];
5-(imidazo [1,2-a] pyridin-6-yl)-N-46-(4-methylpiperazin-1-yl)pyridin-3-yl)methyl)-7H-pyrrolo [2,3-d]pyrimidin-2-amine [138];
6-(5-(imidazo[1,2-a]pyridin-6-yl)-7H-pyrrolo [2,3-d]pyrimidin-2-yl)quinoline [139];
5-(imidazo [1,2-a] pyridin-6-yl)-4-(1-methyl-1H-pyrazol-4-yl)-7H-pyrrolo [2,3-d] pyrimidine [140];
5-(imidazo [1,2-a] pyridin-6-yl)-N-(trans-4-morpholinocyclohexyl)-7H-pyrrolo [2,3-d]pyrimidin-4-amine [141];
5-(3-fluoroimidazo [1,2-a]pyridin-6-yl)-2-isobutyl-7H-pyrrolo [2,3-d]pyrimidine [143];
5-(3-fluoroimidazo [1,2-a]pyridin-6-yl)-2-(3 ,3 ,3-trifluoropropyl)-7H-pyrrolo [2,3-d]pyrimidine [144];
2-cyclopropyl-5-(3-fluoroimidazo [1,2-a] pyridin-6-yl)-7H-pyrrolo [2,3-d]pyrimidine [145];
2-cyclobutyl-5-(3-fluoroimidazo [1,2-a] pyridin-6-yl)-7H-pyrrolo [2,3-d]pyrimidine [146];
2-(cyclobutylmethyl)-5-(3-fluoroimidazo [1,2-a] pyridin-6-yl)-7H-pyrrolo [2,3-d]pyrimidine [147];
5-(3-fluoroimidazo [1,2-a] pyridin-6-yl)-7H-pyrrolo [2,3-d]pyrimidin-2-amine [148];
5-(3-fluoroimidazo [1,2-a]pyridin-6-yl)-N-methyl-7H-pyrrolo [2,3-d]pyrimidin-2-amine [149];
N-ethyl-5-(3-fluoroimidazo [1,2-a] pyridin-6-yl)-7H-pyrrolo [2,3-d]pyrimidin-2-amine [150];
5-(3-fluoroimidazo [1,2-a]pyridin-6-yl)-N-i sopropyl-7H-pyrrolo [2,3-d] pyrimidin-2-amine [151];
5-(3-fluoroimidazo [1,2-a] pyridin-6-yl)-N-i sobutyl-7H-pyrrolo [2,3-d]pyrimidin-2-amine [152];
5-(3-fluoroimidazo [1,2-a] pyridin-6-yl)-N-neopentyl-7H-pyrrolo [2,3-d]pyrimidin-2-amine [153];
(S)-N-(sec-butyl)-5-(3-fluoroimidazo [1,2-a] pyridin-6-yl)-7H-pyrrolo [2,3-d]pyrimidin-2-amine [154];
N-(2-fluoro-2-methylpropyl)-5-(3-fluoroimidazo [1,2-a]pyridin-6-yl)-7H-pyrrolo [2,3-d]pyrimidin-2-amine [155];
N-(2,2-difluoropropyl)-5-(3-fluoroimidazo [1,2-a]pyridin-6-yl)-7H-pyrrolo [2,3-d]pyrimidin-2-amine [156];
5-(3-fluoroimidazo [1,2-a]pyridin-6-yl)-N-(2,2,2-trifluoroethyl)-7H-pyrrolo [2,3-d]pyrimidin-2-amine [157];
(R)-5-(3-fluoroimidazo[1,2-a] pyridin-6-yl)-N-(1, 1, 1-trifluoropropan-2-yl)-7H-pyrrolo [2,3-d]pyrimidin-2-amine [158];
(S)-5-(3-fluoroimidazo[1,2-a] pyridin-6-yl)-N-(1, 1, 1-trifluoropropan-2-yl)-7H-pyrrolo [2,3-d]pyrimidin-2-amine [159];
5-(3-fluoroimidazo [1,2-a]pyridin-6-yl)-N-(3 ,3 ,3-trifluoropropyl)-7H-pyrrolo [2,3-d]pyrimidin-2-amine [160];
(S)-5-(3-fluoroimidazo[1,2-a] pyridin-6-yl)-N-(1-methoxypropan-2-yl)-7H-pyrrolo [2,3-d]pyrimidin-2-amine [161];
2-ethoxy-5-(3-fluoroimidazo [1,2-a] pyridin-6-yl)-7H-pyrrolo [2,3-d]pyrimidine [162];
N-((1-fluorocyclopropyl)methyl)-5-(3-fluoroimidazo [1,2-a]pyridin-6-yl)-7H-pyrrolo [2,3-d]pyrimidin-2-amine [163];
(R)-N-(1-cyclopropylethyl)-5-(3-fluoroimidazo [1,2-a] pyridin-6-yl)-7H-pyrrolo [2,3-d]pyrimidin-2-amine [164];
5-(3-fluoroimidazo [1,2-a]pyridin-6-yl)-N-((1-methylcyclopropyl)methyl)-7H-pyrrolo [2,3-d]pyrimidin-2-amine [165];
5-(3-fluoroimidazo [1,2-a]pyridin-6-yl)-N-4 1-(trifluoromethyl)cyclopropyl)methyl)-7H-pyrrolo [2,3-d]pyrimidin-2-amine [166];
N-(2-cyclopropyl-2,2-difluoroethyl)-5-(3-fluoroimidazo [1,2-a]pyridin-6-yl)-7H-pyrrolo [2,3-d]pyrimidin-2-amine [167];
N-(3,3-difluorocyclobutyl)-5-(3-fluoroimidazo [1,2-a] pyridin-6-yl)-7H-pyrrolo [2,3-d]pyrimidin-2-amine [168];
N-((1-fluorocyclobutyl)methyl)-5-(3-fluoroimidazo [1,2-a] pyridin-6-yl)-7H-pyrrolo [2,3-d]pyrimidin-2-amine [169];
N-(4,4-difluorocyclohexyl)-5-(3-fluoroimidazo [1,2-a]pyridin-6-yl)-7H-pyrrolo [2,3-d] pyrimidin-2-amine [170];
5-(3-fluoroimidazo [1,2-a]pyridin-6-yl)-N-(cis-4-methoxycyclohexyl)-7H-pyrrolo [2,3-d]pyrimidin-2-amine [171];
N-((1-fluorocyclohexyl)methyl)-5-(3-fluoroimidazo [1,2-a]pyridin-6-yl)-7H-pyrrolo [2,3-d]pyrimidin-2-amine [172];
N-((4,4-difluorocyclohexyl)methyl)-5-(3-fluoroimidazo [1,2-a]pyridin-6-yl)-7H-pyrrolo [2,3-d]pyrimidin-2-amine [173];
5-(3-fluoroimidazo [1,2-a]pyridin-6-yl)-N-(1-methylpiperidin-4-yl)-7H-pyrrolo [2,3-d]pyrimidin-2-amine [174];
5-(3-fluoroimidazo [1,2-a]pyridin-6-yl)-N-((1-methylpipe ridin-4-yl)methyl)-7H-pyrrolo [2,3-d]pyrimidin-2-amine [175];
5-(3-fluoroimidazo [1,2-a]pyridin-6-yl)-N-(tetrahydro -2H-pyran-4-yl)-7H-pyrrolo [2,3-d]pyrimidin-2-amine [176];
5-(3-fluoroimidazo [1,2-a]pyridin-6-yl)-N-(3-(4-methylpiperazin-1-yl)phenyl)-7H-pyrrolo [2,3-d]pyrimidin-2-amine [177];
5-(3-fluoroimidazo [1,2-a]pyridin-6-yl)-N-(pyridin-4-yl)-7H-pyrrolo [2,3-d]pyrimidin-2-amine
[178];
5-(3-fluoroimidazo [1,2-a]pyridin-6-yl)-N-(2-(4-methylpiperazin-1-yl)pyridin-4-yl)-7H-pyrrolo [2,3-d]pyrimidin-2-amine [179];
5-(3-fluoroimidazo [1,2-a]pyridin-6-yl)-N-(6-(4-methylpiperazin-1-yl)pyridin-3-yl)-7H-pyrrolo [2,3-d]pyrimidin-2-amine [180];
5-(3-fluoroimidazo [1,2-a]pyridin-6-yl)-N-46-(4-methylpiperazin-1-yl)pyridin-3-yl)methyl)-7H-pyrrolo [2,3-d]pyrimidin-2-amine [181];
6-(5-(3-fluoroimidazo [1,2-a]pyridin-6-yl)-7H-pyrrolo [2,3-d]pyrimidin-2-yl)quinoline [182];
5-(3-fluoroimidazo [1,2-a]pyridin-6-yl)-4-(1-methyl-1H-pyrazol-4-yl)-7H-pyrrolo [2,3-d]pyrimidine [183];
5-(3-fluoroimidazo [1,2-a]pyridin-6-yl)-N-(trans-4-morpholinocyclohexyl)-7H-pyrrolo [2,3-d]pyrimidin-4-amine [184];
5-(8-fluoroimidazo [1,2-a]pyridin-6-yl)-2-isobutyl-7H-pyrrolo [2,3-d]pyrimidine [186];
5-(8-fluoroimidazo [1,2-a]pyridin-6-yl)-2-isobutyl-4-methoxy-7H-pyrrolo [2,3-d]pyrimidine [187];
5-(8-fluoroimidazo [1,2-a]pyridin-6-yl)-2-(3,3,3-trifluoropropyl)-7H-pyrrolo [2,3-d]pyrimidine
[188];
2-cyclopropyl-5-(8-fluoroimidazo [1,2-a]pyridin-6-yl)-7H-pyrrolo [2,3-d]pyrimidine [189];
2-cyclobutyl-5-(8-fluoroimidazo [1,2-a]pyridin-6-yl)-7H-pyrrolo [2,3-d]pyrimidine [190];
2-(cyclobutylmethyl)-5-(8-fluoroimidazo [1,2-a]pyridin-6-yl)-7H-pyrrolo [2,3-d]pyrimidine [191];
5-(8-fluoroimidazo [1,2-a]pyridin-6-yl)-7H-pyrrolo [2,3-d]pyrimidin-2-amine [192];
5-(8-fluoroimidazo [1,2-a]pyridin-6-yl)-N-methyl-7H-pyrrolo [2,3-d]pyrimidin-2-amine [193];
N-ethyl-5-(8-fluoroimidazo [1,2-a]pyridin-6-yl)-7H-pyrrolo [2,3-d]pyrimidin-2-amine [194];
5-(8-fluoroimidazo [1,2-a]pyridin-6-yl)-N-isopropyl-7H-pyrrolo [2,3-d]pyrimidin-2-amine [195];
5-(8-fluoroimidazo [1,2-a]pyridin-6-yl)-N-isobutyl-7H-pyrrolo [2,3-d]pyrimidin-2-amine [196];
5-(8-fluoroimidazo [1,2-a]pyridin-6-yl)-N-neopentyl-7H-pyrrolo [2,3-d]pyrimidin-2-amine [197];
(S)-N-(sec-butyl)-5-(8-fluoroimidazo [1,2-a]pyridin-6-yl)-7H-pyrrolo [2,3-d]pyrimidin-2-amine [198];
N-(2-fluoro-2-methylpropyl)-5-(8-fluoroimidazo [1,2-a] pyridin-6-yl)-7H-pyrrolo [2,3-d]pyrimidin-2-amine [199];
N-(2,2-difluoropropyl)-5-(8-fluoroimidazo [1,2-a]pyridin-6-yl)-7H-pyrrolo [2,3-d]pyrimidin-2-amine [200];
5-(8-fluoroimidazo [1,2-a]pyridin-6-yl)-N-(2,2,2-trifluoroethyl)-7H-pyrrolo [2,3-d]pyrimidin-2-amine [201];
(R)-5-(8-fluoroimidazo [1,2-a]pyridin-6-yl)-N-(1,1,1-trifluoropropan-2-yl)-7H-pyrrolo [2,3-d]pyrimidin-2-amine [202];
(S)-5-(8-fluoroimidazo [1,2-a]pyridin-6-yl)-N-(1,1,1-trifluoropropan-2-yl)-7H-pyrrolo [2,3-d]pyrimidin-2-amine [203];
5-(8-fluoroimidazo [1,2-a]pyridin-6-yl)-N-(3,3,3-trifluoropropyl)-7H-pyrrolo [2,3-d]pyrimidin-2-amine [204];
(S)-5-(8-fluoroimidazo [1,2-a]pyridin-6-yl)-N-(1-methoxypropan-2-yl)-7H-pyrrolo [2,3-d]pyrimidin-2-amine [205];
2-ethoxy-5-(8-fluoroimidazo [1,2-a]pyridin-6-yl)-7H-pyrrolo [2,3-d]pyrimidine [206];
N-(cyclopropylmethyl)-5-(8-fluoroimidazo[1,2-a]pyridin-6-yl)-7H-pyrrolo[2,3-d]pyrimidin-2-amine [207];
N-((1-fluorocyclopropyl)methyl)-5-(8-fluoroimidazo[1,2-a]pyridin-6-yl)-7H-pyrrolo[2,3-d]pyrimidin-2-amine [208];
(R)-N-(1-cyclopropylethyl)-5-(8-fluoroimidazo[1,2-a]pyridin-6-yl)-7H-pyrrolo[2,3-d]pyrimidin-2-amine [209];
5-(8-fluoroimidazo[1,2-a]pyridin-6-yl)-N-((1-methylcyclopropyl)methyl)-7H-pyrrolo[2,3-d]pyrimidin-2-amine [210];
5-(8-fluoroimidazo[1,2-a]pyridin-6-yl)-N-41-(trifluoromethyl)cyclopropyl)methyl)-7H-pyrrolo[2,3-d]pyrimidin-2-amine [211];
N-(2-cyclopropyl-2,2-difluoroethyl)-5-(8-fluoroimidazo[1,2-a]pyridin-6-yl)-7H-pyrrolo[2,3-d]pyrimidin-2-amine [212];
N-(3,3-difluorocyclobutyl)-5-(8-fluoroimidazo[1,2-a]pyridin-6-yl)-7H-pyrrolo[2,3-d]pyrimidin-2-amine [213];
N-((1-fluorocyclobutyl)methyl)-5-(8-fluoroimidazo[1,2-a]pyridin-6-yl)-7H-pyrrolo[2,3-d]pyrimidin-2-amine [214];
N-(4,4-difluorocyclohexyl)-5-(8-fluoroimidazo[1,2-a]pyridin-6-yl)-7H-pyrrolo[2,3-d]pyrimidin-2-amine [215];
5-(8-fluoroimidazo[1,2-a]pyridin-6-yl)-N-(cis-4-methoxycyclohexyl)-7H-pyrrolo[2,3-d]pyrimidin-2-amine [216];
N-((1-fluorocyclohexyl)methyl)-5-(8-fluoroimidazo[1,2-a]pyridin-6-yl)-7H-pyrrolo[2,3-d]pyrimidin-2-amine [217];
N-((4,4-difluorocyclohexyl)methyl)-5-(8-fluoroimidazo[1,2-a]pyridin-6-yl)-7H-pyrrolo[2,3-d]pyrimidin-2-amine [218];
5-(8-fluoroimidazo[1,2-a]pyridin-6-yl)-N-(1-methylpiperidin-4-yl)-7H-pyrrolo[2,3-d]pyrimidin-2-amine [219];
5-(8-fluoroimidazo[1,2-a]pyridin-6-yl)-N-((1-methylpiperidin-4-yl)methyl)-7H-pyrrolo[2,3-d]pyrimidin-2-amine [220];
5-(8-fluoroimidazo[1,2-a]pyridin-6-yl)-N-(tetrahydro-2H-pyran-4-yl)-7H-pyrrolo[2,3-d]pyrimidin-2-amine [221];
5-(8-fluoroimidazo[1,2-a]pyridin-6-yl)-N-(3-(4-methylpiperazin-1-yl)phenyl)-7H-pyrrolo[2,3-d]pyrimidin-2-amine [222];
5-(8-fluoroimidazo[1,2-a]pyridin-6-yl)-N-(pyridin-4-yl)-7H-pyrrolo[2,3-d]pyrimidin-2-amine
[223];
5-(8-fluoroimidazo [1,2-a]pyridin-6-yl)-N-(2-(4-methylpiperazin-1-yl)pyridin-4-yl)-7H-pyrrolo [2,3-d]pyrimidin-2-amine [224];
5-(8-fluoroimidazo [1,2-a]pyridin-6-yl)-N-(6-(4-methylpiperazin-1-yl)pyridin-3-yl)-7H-pyrrolo [2,3-d]pyrimidin-2-amine [225];
5-(8-fluoroimidazo [1,2-a]pyridin-6-yl)-N-((6-(4-methylpiperazin-1-yl)pyridin-3-yl)methyl)-7H-pyrrolo [2,3-d]pyrimidin-2-amine [226];
6-(5-(8-fluoroimidazo [1,2-a]pyridin-6-yl)-7H-pyrrolo [2,3-d]pyrimidin-2-yl)quinoline [227];
5-(8-fluoroimidazo [1,2-a]pyridin-6-yl)-4-(1-methyl-1H-pyrazol-4-yl)-7H-pyrrolo [2,3-d]pyrimidine [228];
5-(8-fluoroimidazo [1,2-a]pyridin-6-yl)-N-(trans-4-morpholinocyclohexyl)-7H-pyrrolo [2,3-d]pyrimidin-4-amine [229];
5-(8-fluoro-2-methylimidazo [1,2-a] pyridin-6-yl)-2-i sobutyl-7H-pyrrolo [2,3-d]pyrimidine [231];
5-(8-fluoro-2-methylimidazo [1,2-a]pyridin-6-yl)-2-(3,3,3-trifluoropropyl)-7H-pyrrolo [2,3-d]pyrimidine [232];
2-cyclopropyl-5-(8-fluoro-2-methylimidazo [1,2-a]pyridin-6-yl)-7H-pyrrolo [2,3-d]pyrimidine [233];
2-cyclobutyl-5-(8-fluoro-2-methylimidazo [1,2-a] pyridin-6-yl)-7H-pyrrolo [2,3-d]pyrimidine
[234];
2-(cyclobutylmethyl)-5-(8-fluoro-2-methylimidazo [1,2-a]pyridin-6-yl)-7H-pyrrolo [2,3-d]pyrimidine [235];
5-(8-fluoro-2-methylimidazo [1,2-a] pyridin-6-yl)-7H-pyrrolo [2,3-d]pyrimidin-2-amine [236];
5-(8-fluoro-2-methylimidazo [1,2-a] pyridin-6-yl)-N-methyl-7H-pyrrolo [2,3-d]pyrimidin-2-amine
[237];
N-ethyl-5-(8-fluoro-2-methylimidazo [1,2-a]pyridin-6-yl)-7H-pyrrolo [2,3-d]pyrimidin-2-amine
[238];
5-(8-fluoro-2-methylimidazo [1,2-a] pyridin-6-yl)-N-i sopropyl-7H-pyrrolo [2,3-d]pyrimidin-2-amine [239];
5-(8-fluoro-2-methylimidazo [1,2-a] pyridin-6-yl)-N-i sobutyl-7H-pyrrolo [2,3-d]pyrimidin-2-amine
[240];
5-(8-fluoro-2-methylimidazo [1,2-a] pyridin-6-yl)-N-neopentyl-7H-pyrrolo [2,3-d]pyrimidin-2-amine [241];
(S)-N-(sec-butyl)-5-(8-fluoro-2-methylimidazo [1,2-a]pyridin-6-yl)-7H-pyrrolo [2,3-d]pyrimidin-2-amine [242];
5-(8-fluoro-2-methylimidazo[1,2-a]pyridin-6-yl)-N-(2-fluoro-2-methylpropyl)-7H-pyrrolo[2,3-d]pyrimidin-2-amine [243];
N-(2,2-difluoropropyl)-5-(8-fluoro-2-methylimidazo[1,2-a]pyridin-6-yl)-7H-pyrrolo[2,3-d]pyrimidin-2-amine [244];
5-(8-fluoro-2-methylimidazo[1,2-a]pyridin-6-yl)-N-(2,2,2-trifluoroethyl)-7H-pyrrolo[2,3-d]pyrimidin-2-amine [245];
(R)-5-(8-fluoro-2-methylimidazo[1,2-a]pyridin-6-yl)-N-(1,1,1-trifluoropropan-2-yl)-7H-pyrrolo[2,3-d]pyrimidin-2-amine [246];
(S)-5-(8-fluoro-2-methylimidazo[1,2-a]pyridin-6-yl)-N-(1,1,1-trifluoropropan-2-yl)-7H-pyrrolo[2,3-d]pyrimidin-2-amine [247];
5-(8-fluoro-2-methylimidazo[1,2-a]pyridin-6-yl)-N-(3,3,3-trifluoropropyl)-7H-pyrrolo[2,3-d]pyrimidin-2-amine [248];
(S)-5-(8-fluoro-2-methylimidazo[1,2-a]pyridin-6-yl)-N-(1-methoxypropan-2-yl)-7H-pyrrolo[2,3-d]pyrimidin-2-amine [249];
2-ethoxy-5-(8-fluoro-2-methylimidazo[1,2-a]pyridin-6-yl)-7H-pyrrolo[2,3-d]pyrimidine [250];
5-(8-fluoro-2-methylimidazo[1,2-a]pyridin-6-yl)-N-((1-fluorocyclopropyl)methyl)-7H-pyrrolo[2,3-d]pyrimidin-2-amine [251];
(R)-N-(1-cyclopropylethyl)-5-(8-fluoro-2-methylimidazo[1,2-a]pyridin-6-yl)-7H-pyrrolo[2,3-d]pyrimidin-2-amine [252];
5-(8-fluoro-2-methylimidazo[1,2-a]pyridin-6-yl)-N-((1-methylcyclopropyl)methyl)-7H-pyrrolo[2,3-d]pyrimidin-2-amine [253];
5-(8-fluoro-2-methylimidazo[1,2-a]pyridin-6-yl)-N-41-(trifluoromethyl)cyclopropyl)methyl)-7H-pyrrolo[2,3-d]pyrimidin-2-amine [254];
N-(2-cyclopropyl-2,2-difluoroethyl)-5-(8-fluoro-2-methylimidazo[1,2-a]pyridin-6-yl)-7H-pyrrolo[2,3-d]pyrimidin-2-amine [255];
N-(3,3-difluorocyclobutyl)-5-(8-fluoro-2-methylimidazo[1,2-a]pyridin-6-yl)-7H-pyrrolo[2,3-d]pyrimidin-2-amine [256];
5-(8-fluoro-2-methylimidazo[1,2-a]pyridin-6-yl)-N-((1-fluorocyclobutypmethyl)-7H-pyrrolo[2,3-d]pyrimidin-2-amine [257];
N-(4,4-difluorocyclohexyl)-5-(8-fluoro-2-methylimidazo[1,2-a]pyridin-6-yl)-7H-pyrrolo[2,3-d]pyrimidin-2-amine [258];
5-(8-fluoro-2-methylimidazo[1,2-a]pyridin-6-yl)-N-(cis-4-methoxycyclohexyl)-7H-pyrrolo[2,3-d]pyrimidin-2-amine [259];
5-(8-fluoro-2-methylimidazo [1,2-a] pyridin-6-yl)-N-((1-fluorocyclohexyl)me thyl)-7H-pyrrolo [2,3-d]pyrimidin-2-amine [260];
N-((4,4-difluorocyclohexyl)methyl)-5-(8-fluoro-2-methylimidazo [1,2-a]pyridin-6-yl)-7H-pyrrolo [2,3-d]pyrimidin-2-amine [261];
5-(8-fluoro-2-methylimidazo [1,2-a] pyridin-6-yl)-N-(1-methylpiperidin-4-yl)-7H-pyrrolo [2,3-d]pyrimidin-2-amine [262];
5-(8-fluoro-2-methylimidazo [1,2-a] pyridin-6-yl)-N-((1-methylpiperidin-4-yl)methyl)-7H-pyrrolo [2,3-d]pyrimidin-2-amine [263];
5-(8-fluoro-2-methylimidazo [1,2-a] pyridin-6-yl)-N-(tetrahydro-2H-pyran-4-yl)-7H-pyrrolo [2,3-d]pyrimidin-2-amine [264];
5-(8-fluoro-2-methylimidazo [1,2-a] pyridin-6-yl)-N-(3-(4-me thylpiperazin-1-yl)phenyl)-7H-pyrrolo [2,3-d]pyrimidin-2-amine [265];
5-(8-fluoro-2-methylimidazo [1,2-a] pyridin-6-yl)-N-(pyridin-4-yl)-7H-pyrrolo [2,3-d]pyrimidin-2-amine [266];
5-(8-fluoro-2-methylimidazo [1,2-a] pyridin-6-yl)-N-(2-(4-methylpipe razin-1-yl)pyridin-4-yl)-7H-pyrrolo [2,3-d]pyrimidin-2-amine [267];
5-(8-fluoro-2-methylimidazo [1,2-a] pyridin-6-yl)-N-(6-(4-methylpipe razin-1-yl)pyridin-3-yl)-7H-pyrrolo [2,3-d]pyrimidin-2-amine [268];
5-(8-fluoro-2-methylimidazo [1,2-a] pyridin-6-yl)-N-46-(4-methylpiperazin-1-yl)pyridin-3-yl)methyl)-7H-pyrrolo [2,3-d]pyrimidin-2-amine [269];
6-(5-(8-fluoro-2-methylimidazo [1,2-a]pyridin-6-yl)-7H-pyrrolo [2,3-d]pyrimidin-2-yl)quinoline
[270];
5-(8-fluoro-2-methylimidazo [1,2-a] pyridin-6-yl)-4-(1-methyl-1H-pyrazol-4-yl)-7H-pyrrolo [2,3-d]pyrimidine [271];
5-(8-fluoro-2-methylimidazo [1,2-a] pyridin-6-yl)-N-(trans-4-morpholinocyclohexyl)-7H-pyrrolo [2,3-d]pyrimidin-4-amine [272];
5-(8-fluoro-3-methylimidazo [1,2-a] pyridin-6-yl)-24 sobutyl-7H-pyrrolo [2,3-d]pyrimidine [274];
5-(8-fluoro-3-methylimidazo [1,2-a] pyridin-6-yl)-2-(3,3,3-trifluoropropyl)-7H-pyrrolo [2,3-d]pyrimidine [275];
2-cyclopropyl-5-(8-fluoro-3-methylimidazo [1,2-a]pyridin-6-yl)-7H-pyrrolo [2,3-d]pyrimidine
[276];
2-cyclobutyl-5-(8-fluoro-3-methylimidazo [1,2-a] pyridin-6-yl)-7H-pyrrolo [2,3-d]pyrimidine
[277];
2-(cyclobutylmethyl)-5-(8-fluoro-3-methylimidazo[1,2-a]pyridin-6-yl)-7H-pyrrolo[2,3-d]pyrimidine [278];
5-(8-fluoro-3-methylimidazo[1,2-a]pyridin-6-yl)-7H-pyrrolo[2,3-d]pyrimidin-2-amine [279];
5-(8-fluoro-3-methylimidazo[1,2-a]pyridin-6-yl)-N-methyl-7H-pyrrolo[2,3-d]pyrimidin-2-amine ;
N-ethyl-5-(8-fluoro-3-methylimidazo[1,2-a]pyridin-6-yl)-7H-pyrrolo[2,3-d]pyrimidin-2-amine ;
5-(8-fluoro-3-methylimidazo[1,2-a]pyridin-6-yl)-N-isopropyl-7H-pyrrolo[2,3-d]pyrimidin-2-amine [282];
5-(8-fluoro-3-methylimidazo[1,2-a]pyridin-6-yl)-N-isobutyl-7H-pyrrolo[2,3-d]pyrimidin-2-amine ;
5-(8-fluoro-3-methylimidazo[1,2-a]pyridin-6-yl)-N-neopentyl-7H-pyrrolo[2,3-d]pyrimidin-2-amine [284];
(S)-N-(sec-butyl)-5-(8-fluoro-3-methylimidazo[1,2-a]pyridin-6-yl)-7H-pyrrolo[2,3-d]pyrimidin-2-amine [285];
N-(2-fluoro-2-methylpropyl)-5-(8-fluoro-3-methylimidazo[1,2-a]pyridin-6-yl)-7H-pyrrolo[2,3-d]pyrimidin-2-amine [286];
N-(2,2-difluoropropyl)-5-(8-fluoro-3-methylimidazo[1,2-a]pyridin-6-yl)-7H-pyrrolo[2,3-d]pyrimidin-2-amine [287];
5-(8-fluoro-3-methylimidazo[1,2-a]pyridin-6-yl)-N-(2,2,2-trifluoroethyl)-7H-pyrrolo[2,3-d]pyrimidin-2-amine [288];
(R)-5-(8-fluoro-3-methylimidazo[1,2-a]pyridin-6-yl)-N-(1,1,1-trifluoropropan-2-yl)-7H-pyrrolo[2,3-d]pyrimidin-2-amine [289];
(S)-5-(8-fluoro-3-methylimidazo[1,2-a]pyridin-6-yl)-N-(1,1,1-trifluoropropan-2-yl)-7H-pyrrolo[2,3-d]pyrimidin-2-amine [290];
5-(8-fluoro-3-methylimidazo[1,2-a]pyridin-6-yl)-N-(3,3,3-trifluoropropyl)-7H-pyrrolo[2,3-d]pyrimidin-2-amine [291];
(S)-5-(8-fluoro-3-methylimidazo[1,2-a]pyridin-6-yl)-N-(1-methoxypropan-2-yl)-7H-pyrrolo[2,3-d]pyrimidin-2-amine [292];
2-ethoxy-5-(8-fluoro-3-methylimidazo[1,2-a]pyridin-6-yl)-7H-pyrrolo[2,3-d]pyrimidine [293];
5-(8-fluoro-3-methylimidazo[1,2-a]pyridin-6-yl)-N-((1-fluorocyclopropyl)methyl)-7H-pyrrolo[2,3-d]pyrimidin-2-amine [294];
(R)-N-(1-cyclopropylethyl)-5-(8-fluoro-3-methylimidazo[1,2-a]pyridin-6-yl)-7H-pyrrolo[2,3-d]pyrimidin-2-amine [295];
5-(8-fluoro-3-methylimidazo[1,2-a]pyridin-6-yl)-N-((1-methylcyclopropyl)methyl)-7H-pyrrolo[2,3-d]pyrimidin-2-amine [296];
5-(8-fluoro-3-methylimidazo[1,2-a]pyridin-6-yl)-N-41-(trifluoromethyl)cyclopropyl)methyl)-7H-pyrrolo[2,3-d]pyrimidin-2-amine [297];
N-(2-cyclopropyl-2,2-difluoroethyl)-5-(8-fluoro-3-methylimidazo[1,2-a]pyridin-6-yl)-7H-pyrrolo[2,3-d]pyrimidin-2-amine [298];
N-(3,3-difluorocyclobutyl)-5-(8-fluoro-3-methylimidazo[1,2-a]pyridin-6-yl)-7H-pyrrolo[2,3-d]pyrimidin-2-amine [299];
5-(8-fluoro-3-methylimidazo[1,2-a]pyridin-6-yl)-N-((1-fluorocyclobutypmethyl)-7H-pyrrolo[2,3-d]pyrimidin-2-amine [300];
N-(4,4-difluorocyclohexyl)-5-(8-fluoro-3-methylimidazo[1,2-a]pyridin-6-yl)-7H-pyrrolo[2,3-d]pyrimidin-2-amine [301];
5-(8-fluoro-3-methylimidazo[1,2-a]pyridin-6-yl)-N-(cis-4-methoxycyclohexyl)-7H-pyrrolo[2,3-d]pyrimidin-2-amine [302];
5-(8-fluoro-3-methylimidazo[1,2-a]pyridin-6-yl)-N-((1-fluorocyclohexyl)methyl)-7H-pyrrolo[2,3-d]pyrimidin-2-amine [303];
N-((4,4-difluorocyclohexyl)methyl)-5-(8-fluoro-3-methylimidazo[1,2-a]pyridin-6-yl)-7H-pyrrolo[2,3-d]pyrimidin-2-amine [304];
5-(8-fluoro-3-methylimidazo[1,2-a]pyridin-6-yl)-N-(1-methylpiperidin-4-yl)-7H-pyrrolo[2,3-d]pyrimidin-2-amine [305];
5-(8-fluoro-3-methylimidazo[1,2-a]pyridin-6-yl)-N-((1-methylpiperidin-4-yl)methyl)-7H-pyrrolo[2,3-d]pyrimidin-2-amine [306];
5-(8-fluoro-3-methylimidazo[1,2-a]pyridin-6-yl)-N-(tetrahydro-2H-pyran-4-yl)-7H-pyrrolo[2,3-d]pyrimidin-2-amine [307];
5-(8-fluoro-3-methylimidazo[1,2-a]pyridin-6-yl)-N-(3-(4-methylpiperazin-1-yl)phenyl)-7H-pyrrolo[2,3-d]pyrimidin-2-amine [308];
5-(8-fluoro-3-methylimidazo[1,2-a]pyridin-6-yl)-N-(pyridin-4-yl)-7H-pyrrolo[2,3-d]pyrimidin-2-amine [309];
5-(8-fluoro-3-methylimidazo[1,2-a]pyridin-6-yl)-N-(2-(4-methylpiperazin-1-yl)pyridin-4-yl)-7H-pyrrolo[2,3-d]pyrimidin-2-amine [310];
5-(8-fluoro-3-methylimidazo[1,2-a]pyridin-6-yl)-N-(6-(4-methylpiperazin-1-yl)pyridin-3-yl)-7H-pyrrolo[2,3-d]pyrimidin-2-amine [311];
5-(8-fluoro-3-methylimidazo[1,2-a]pyridin-6-yl)-N-46-(4-methylpiperazin-1-yl)pyridin-3-yl)methyl)-7H-pyrrolo[2,3-d]pyrimidin-2-amine [312];
6-(5-(8-fluoro-3-methylimidazo [1,2-a]pyridin-6-yl)-7H-pyrrolo [2,3-d]pyrimidin-2-yl)quinoline [313];
5-(8-fluoro-3-methylimidazo [1,2-a] pyridin-6-yl)-4-(1-methyl-1H-pyrazol-4-yl)-7H-pyrrolo [2,3-d]pyrimidine [314];
5-(8-fluoro-3-methylimidazo [1,2-a] pyridin-6-yl)-N-(trans-4-morpholinocyclohexyl)-7H-pyrrolo [2,3-d]pyrimidin-4-amine [315];
5-(3-(difluoromethyl)imidazo[1,2-a]pyridin-6-yl)-2-isobutyl-7H-pyrrolo [2,3-d]pyrimidine [317];
5-(3-(difluoromethyl)imidazo[1,2-a]pyridin-6-yl)-2-(3,3,3-trifluoropropyl)-7H-pyrrolo [2,3-d]pyrimidine [318];
2-cyclopropyl-5-(3-(difluoromethyl)imidazo[1,2-a]pyridin-6-yl)-7H-pyrrolo [2,3-d]pyrimidine [319];
2-cyclobutyl-5-(3-(difluoromethyl)imidazo[1,2-a]pyridin-6-yl)-7H-pyrrolo [2,3-d]pyrimidine
[320];
2-(cyclobutylmethyl)-5-(3-(difluoromethyl)imidazo [1,2-a]pyridin-6-yl)-7H-pyrrolo [2,3-d]pyrimidine
[321];
5-(3-(difluoromethyl)imidazo[1,2-a]pyridin-6-yl)-7H-pyrrolo [2,3-d]pyrimidin-2-amine [322];
5-(3-(difluoromethyl)imidazo[1,2-a]pyridin-6-yl)-N-methyl-7H-pyrrolo [2,3-d] pyrimidin-2-amine
[323];
5-(3-(difluoromethyl)imidazo[1,2-a]pyridin-6-yl)-N-ethyl-7H-pyrrolo [2,3-d]pyrimidin-2-amine
[324];
5-(3-(difluoromethyl)imidazo[1,2-a]pyridin-6-yl)-N-isopropyl-7H-pyrrolo [2,3-d] pyrimidin-2-amine [325];
5-(3-(difluoromethyl)imidazo[1,2-a]pyridin-6-yl)-N-isobutyl-7H-pyrrolo [2,3-d]pyrimidin-2-amine [326];
5-(3-(difluoromethyl)imidazo[1,2-a]pyridin-6-yl)-N-neopentyl-7H-pyrrolo [2,3-d]pyrimidin-2-amine [327];
(S)-N-(sec-butyl)-5-(3-(difluoromethyl)imidazo [1,2-a] pyridin-6-yl)-7H-pyrrolo [2,3-d]pyrimidin-2-amine [328];
5-(3-(difluoromethyl)imidazo[1,2-a]pyridin-6-yl)-N-(2-fluoro-2-methylpropyl)-7H-pyrrolo [2,3-d]pyrimidin-2-amine [329];
5-(3-(difluoromethyl)imidazo[1,2-a]pyridin-6-yl)-N-(2,2-difluoropropyl)-7H-pyrrolo [2,3-d]pyrimidin-2-amine [330];
5-(3-(difluoromethyl)imidazo [1,2-a]pyridin-6-yl)-N-(2,2,2-trifluoroethyl)-7H-pyrrolo [2,3-d]pyrimidin-2-amine [331];
(R)-5-(3-(difluoromethyl)imidazo [1,2-a] pyridin-6-yl)-N-(1, 1, 1-trifluoropropan-2-yl)-7H-pyrrolo [2,3-d]pyrimidin-2-amine [332];
(S)-5-(3-(difluoromethyl)imidazo[1,2-a]pyridin-6-yl)-N-(1, 1, 1-trifluoropropan-2-yl)-7H-pyrrolo [2,3-d]pyrimidin-2-amine [333];
5-(3-(difluoromethyl)imidazo[1,2-a]pyridin-6-yl)-N-(3 ,3 ,3-trifluoropropyl)-7H-pyrrolo [2,3-d]pyrimidin-2-amine [334];
(S)-5-(3-(difluoromethyl)imidazo[1,2-a]pyridin-6-yl)-N-(1-methoxypropan-2-yl)-7H-pyrrolo [2,3-d]pyrimidin-2-amine [335];
5-(3-(difluoromethyl)imidazo[1,2-a]pyridin-6-yl)-2-ethoxy-7H-pyrrolo [2,3-d]pyrimidine [336];
5-(3-(difluoromethyl)imidazo[1,2-a]pyridin-6-yl)-N-((1-fluorocyclopropyl)methyl)-7H-pyrrolo [2,3-d]pyrimidin-2-amine [337];
(R)-N-(1-cyclopropylethyl)-5-(3-(difluoromethyl)imidazo [1,2-a] pyridin-6-yl)-7H-pyrrolo [2,3-d]pyrimidin-2-amine [338];
5-(3-(difluoromethyl)imidazo[1,2-a]pyridin-6-yl)-N-((1-methylcyclopropyl)methyl)-7H-pyrrolo [2,3-d]pyrimidin-2-amine [339];
5-(3-(difluoromethyl)imidazo[1,2-a]pyridin-6-yl)-N-4 1-(trifluoromethyl)cyclopropyl)methyl)-7H-pyrrolo [2,3-d]pyrimidin-2-amine [340];
N-(2-cyclopropyl-2,2-difluoroethyl)-5-(3-(difluoromethyl)imidazo [1,2-a] pyridin-6-yl)-7H-pyrrolo [2,3-d]pyrimidin-2-amine [341];
N-(3,3-difluorocyclobutyl)-5-(3-(difluoromethyl)imidazo[1,2-a]pyridin-6-yl)-7H-pyrrolo [2,3-d]pyrimidin-2-amine [342];
5-(3-(difluoromethyl)imidazo[1,2-a]pyridin-6-yl)-N-((1-fluorocyclobutypmethyl)-7H-pyrrolo [2,3-d]pyrimidin-2-amine [343];
N-(4,4-difluorocyclohexyl)-5-(3-(difluoromethyl)imidazo [1,2-a]pyridin-6-yl)-7H-pyrrolo [2,3-d]pyrimidin-2-amine [344];
5-(3-(difluoromethyl)imidazo[1,2-a]pyridin-6-yl)-N-(cis-4-methoxycyclohexyl)-7H-pyrrolo [2,3-d]pyrimidin-2-amine [345];
5-(3-(difluoromethyl)imidazo [1,2-a]pyridin-6-yl)-N-((1-fluorocyclohexyl)methyl)-7H-pyrrolo [2,3-d]pyrimidin-2-amine [346];
N-((4,4-difluorocyclohexyl)methyl)-5-(3-(difluorome thyl)imidazo [1,2-a] pyridin-6-yl)-7H-pyrrolo [2,3-d]pyrimidin-2-amine [347];
5-(3-(difluoromethyl)imidazo [1,2-a]pyridin-6-yl)-N-(1-methylpiperidin-4-yl)-7H-pyrrolo [2,3-d]pyrimidin-2-amine [348];
5-(3-(difluoromethyl)imidazo[1,2-a]pyridin-6-yl)-N-((1-methylpiperidin-4-yl)methyl)-7H-pyrrolo [2,3-d]pyrimidin-2-amine [349];
5-(3-(difluoromethyl)imidazo[1,2-a]pyridin-6-yl)-N-(tetrahydro-2H-pyran-4-yl)-7H-pyrrolo [2,3-d]pyrimidin-2-amine [350];
5-(3-(difluoromethyl)imidazo[1,2-a]pyridin-6-yl)-N-(3-(4-methylpiperazin-1-yl)phenyl)-7H-pyrrolo [2,3-d]pyrimidin-2-amine [351];
5-(3-(difluoromethyl)imidazo[1,2-a]pyridin-6-yl)-N-(pyridin-4-yl)-7H-pyrrolo [2,3-d]pyrimidin-2-amine [352];
5-(3-(difluoromethyl)imidazo[1,2-a]pyridin-6-yl)-N-(2-(4-methylpipe razin-1-yl)pyridin-4-yl)-7H-pyrrolo [2,3-d]pyrimidin-2-amine [353];
5-(3-(difluoromethyl)imidazo [1,2-a]pyridin-6-yl)-N-(6-(4-methylpipe razin-1-yl)pyridin-3-yl)-7H-pyrrolo [2,3-d]pyrimidin-2-amine [354];
5-(3-(difluoromethyl)imidazo[1,2-a]pyridin-6-yl)-N-46-(4-me thylpiperazin-1-yl)pyridin-3-yl)methyl)-7H-pyrrolo [2,3-d]pyrimidin-2-amine [355];
6-(5-(3-(difluoromethyl)imidazo [1,2-a]pyridin-6-yl)-7H-pyrrolo [2,3-d]pyrimidin-2-yl)quinoline
[356];
5-(3-(difluoromethyl)imidazo[1,2-a]pyridin-6-yl)-44 1-methyl-1H-pyrazol-4-yl)-7H-pyrrolo [2,3-d]pyrimidine [357];
5-(3-(difluoromethyl)imidazo[1,2-a]pyridin-6-yl)-N-((trans-4-morpho linocyclohexyl)-7H-pyrrolo [2,3-d]pyrimidin-4-amine [358];
2-cyclopropyl-5-(3-(difluoromethyl)-8-fluoroimidazo [1,2-a]pyridin-6-yl)-7H-pyrrolo [2,3-d]pyrimidine [359];
(8-fluoro-6-(2-isobutyl-7H-pyrrolo [2,3-d]pyrimidin-5-yl)imidazo [1 ,2-a]pyridin-3-yl)methanol [361];
(8-fluoro-6-(2-(3 ,3 ,3-trifluoropropyl)-7H-pyrrolo [2,3-d]pyrimidin-5-yl)imidazo[1,2-a]pyridin-3-yl)methanol [362];
(6-(2-cyclopropyl-7H-pyrrolo [2,3-d]pyrimidin-5-yl)-8-fluoroimidazo [1,2-a]pyridin-3-yl)methanol [363];
(6-(2-cyclobutyl-7H-pyrrolo [2,3-d]pyrimidin-5-yl)-8-fluoroimidazo [1,2-a]pyridin-3-yl)methanol [364];
(6-(2-(cyclobutylmethyl)-7H-pyrrolo [2,3-d]pyrimidin-5-yl)-8-fluoroimidazo [1,2-a]pyridin-3-yl)methanol [365];
(6-(2-amino-7H-pyrrolo [2,3-d]pyrimidin-5-yl)-8-fluoroimidazo [1,2-a] pyridin-3-yl)methanol [366];
(8-fluoro-6-(2-(methylamino)-7H-pyrrolo[2,3-d]pyrimidin-5-yl)imidazo[1,2-a]pyridin-3-yl)methanol [367];
(6-(2-(ethylamino)-7H-pyrrolo[2,3-d]pyrimidin-5-yl)-8-fluoroimidazo[1,2-a]pyridin-3-yl)methanol [368];
(8-fluoro-6-(2-(isopropylamino)-7H-pyrrolo[2,3-d]pyrimidin-5-y0imidazo[1,2-a]pyridin-3-yl)methanol [369];
(8-fluoro-6-(2-(isobutylamino)-7H-pyrrolo[2,3-d]pyrimidin-5-yl)imidazo[1,2-a]pyridin-3-yl)methanol [370];
(8-fluoro-6-(2-(neopentylamino)-7H-pyrrolo[2,3-d]pyrimidin-5-yl)imidazo[1,2-a]pyridin-3-yl)methanol [371];
(S)-(6-(2-(sec-butylamino)-7H-pyrrolo[2,3-d]pyrimidin-5-yl)-8-fluoroimidazo[1,2-a]pyridin-3-yl)methanol [372];
(8-fluoro-6-(2-((2-fluoro-2-methylpropyl)amino)-7H-pyrrolo[2,3-d]pyrimidin-5-y0imidazo[1,2-a]pyridin-3-yl)methanol [373];
(6-(2-((2,2-difluoropropyl)amino)-7H-pyrrolo[2,3-d]pyrimidin-5-yl)-8-fluoroimidazo[1,2-a]pyridin-3-yl)methanol [374];
(8-fluoro-6-(2-((2,2,2-trifluoroethyl)amino)-7H-pyrrolo[2,3-d]pyrimidin-5-yl)imidazo[1,2-a]pyridin-3-yl)methanol [375];
(R)-(8-fluoro-6-(2-((1,1,1-trifluoropropan-2-yl)amino)-7H-pyrrolo[2,3-d]pyrimidin-5-yl)imidazo[1,2-a]pyridin-3-yl)methanol [376];
(S)-(8-fluoro-6-(2-((1,1,1-trifluoropropan-2-yl)amino)-7H-pyrrolo[2,3-d]pyrimidin-5-yl)imidazo[1,2-a]pyridin-3-yl)methanol [377];
(8-fluoro-6-(2-((3,3,3-trifluoropropyl)amino)-7H-pyrrolo[2,3-d]pyrimidin-5-yl)imidazo[1,2-a]pyridin-3-yl)methanol [378];
(S)-(8-fluoro-6-(2-((l-methoxypropan-2-yl)amino)-7H-pyrrolo[2,3-d]pyrimidin-5-yl)imidazo[1,2-a]pyridin-3-yl)methanol [379];
(6-(2-ethoxy-7H-pyrrolo[2,3-d]pyrimidin-5-yl)-8-fluoroimidazo[1,2-a]pyridin-3-yl)methanol [380];
(8-fluoro-6-(2-(((1-fluorocyclopropyl)methyl)amino)-7H-pyrrolo[2,3-d]pyrimidin-5-yl)imidazo[1,2-a]pyridin-3-yl)methanol [381];
(R)-(6-(2-((l-cyclopropylethyl)amino)-7H-pyrrolo[2,3-d]pyrimidin-5-yl)-8-fluoroimidazo[1,2-a]pyridin-3-yl)methanol [382];
(8-fluoro-6-(2-(((l-methylcyclopropyl)methyl)amino)-7H-pyrrolo[2,3-d]pyrimidin-5-yl)imidazo[1,2-a]pyridin-3-yl)methanol [383];
(8-fluoro-6-(2-(((1-(trifluoromethyl)cyclopropyl)methyl)amino)-7H-pyrrolo [2,3-d]pyrimidin-5-yl)imidazo [1,2-a]pyridin-3-yl)methanol [384];
(6-(2-((2-cyclopropyl-2,2-difluoroethypamino)-7H-pyrrolo [2,3-d]pyrimidin-5-yl)-8-fluoroimidazo [1,2-a]pyridin-3-yl)methanol [385];
(6-(2-((3,3-difluorocyclobutypamino)-7H-pyrrolo [2,3-d] pyrimidin-5-yl)-8-fluoroimidazo [1,2-a] pyridin-3-yl)methanol [386];
(8-fluoro-6-(2-(((1-fluorocyclobutypmethypamino)-7H-pyrrolo [2,3-d]pyrimidin-5-yl)imidazo [1,2-a]pyridin-3-yl)methanol [387];
(6-(2-((4,4-difluorocyclohexyl)amino)-7H-pyrrolo [2,3-d]pyrimidin-5-yl)-8-fluoroimidazo [1,2-a] pyridin-3-yl)methanol [388];
(8-fluoro-6-(2-((cis-4-methoxycyclohexyl)amino)-7H-pyrrolo [2,3-d]pyrimidin-5-y0imidazo [1,2-a] pyridin-3-yl)methanol [389];
(8-fluoro-6-(2-(((1-fluorocyclohexyl)methyl)amino)-7H-pyrrolo [2,3-d] pyrimidin-5-yl)imidazo [1,2-a]pyridin-3-yl)methanol [390];
(6-(2-(((4,4-difluorocyclohexyl)methyl)amino)-7H-pyrrolo [2,3-d] pyrimidin-5-yl)-8-fluoroimidazo [1,2-a]pyridin-3-yl)methanol [391];
(8-fluoro-6-(2-((l-methylpiperidin-4-yl)amino)-7H-pyrrolo [2,3-d]pyrimidin-5-yl)imidazo [1,2-a] pyridin-3-yl)methanol [392];
(8-fluoro-6-(2-(((l-methylpipe ridin-4-yl)methyl)amino)-7H-pyrrolo [2,3-d]pyrimidin-5-yl)imidazo [1,2-a]pyridin-3-yl)methanol [393];
(8-fluoro-6-(2-((tetrahydro-2H-pyran-4-yl)amino)-7H-pyrrolo [2,3-d]pyrimidin-5-yl)imidazo [1,2-a] pyridin-3-yl)methanol [394];
(8-fluoro-6-(2-((3-(4-me thylpiperazin-1-yl)phenyl)amino)-7H-pyrrolo [2,3-d]pyrimidin-5-yl)imidazo [1,2-a]pyridin-3-yl)methanol [395];
(8-fluoro-6-(2-(pyridin-4-ylamino)-7H-pyrrolo [2,3-d]pyrimidin-5-yl)imidazo [1,2-a]pyridin-3-yl)methanol [396];
(8-fluoro-6-(2-((2-(4-me thylpiperazin-1-yl)pyridin-4-yl)amino)-7H-pyrrolo [2,3-d]pyrimidin-5-yl)imidazo [1,2-a]pyridin-3-yl)methanol [397];
(8-fluoro-6-(2-((6-(4-me thylpiperazin-1-yl)pyridin-3-yl)amino)-7H-pyrrolo [2,3-d]pyrimidin-5-yl)imidazo [1,2-a]pyridin-3-yl)methanol [398];
(8-fluoro-6-(2-(((6-(4-methylpiperazin-1-yl)pyridin-3-yl)me thyl)amino)-7H-pyrrolo [2,3-d]pyrimidin-5-yl)imidazo [1,2-a]pyridin-3-yl)methanol [399];
(8-fluoro-6-(2-(quinolin-6-yl)-7H-pyrrolo [2,3-d] pyrimidin-5-yl)imidazo [1,2-a]pyridin-3-yl)methanol [400];
(8-fluoro-6-(4-(1-methyl-1H-pyrazol-4-yl)-7H-pyrrolo [2,3-d] pyrimidin-5-yl)imidazo [1,2-a] pyridin-3-yl)methanol [401];
(8-fluoro-6-(4-((trans-4-morpholinocyclohexyl)amino)-7H-pyrrolo [2,3-d]pyrimidin-5-yl)imidazo[1,2-a]pyridin-3-yl)methanol [402];
2-cyclopropyl-5-(8-fluoro-3-(1-methylpiperidin-4-yl)imidazo[1,2-a]pyridin-6-yl)-7H-pyrrolo [2,3-d]pyrimidine [403];
N-(2,2-difluoroethyl)-6-(24 sobutyl-7H-pyrrolo [2,3-d]pyrimidin-5-yl)imidazo [1,2-a]pyridine-3-carboxamide [405];
N-(2,2-difluoroethyl)-6-(2-(3,3,3-trifluoropropyl)-7H-pyrrolo [2,3-d] pyrimidin-5-y0imidazo [1,2-a] pyridine-3-carboxamide [406];
6-(2-cyclopropyl-7H-pyrrolo [2,3-d]pyrimidin-5-yl)-N-(2,2-difluoroethyl)imidazo [1,2-a] pyridine-3-carboxamide [407];
6-(2-cyclobutyl-7H-pyrrolo [2,3-d] pyrimidin-5-yl)-N-(2,2-difluoroethyl)imidazo [1,2-a]pyridine-3-carboxamide [408];
6-(2-(cyclobutylmethyl)-7H-pyrrolo [2,3-d]pyrimidin-5-yl)-N-(2,2-difluoroethyl)imidazo [1,2-a] pyridine-3-carboxamide [409];
6-(2-amino-7H-pyrrolo [2,3-d] pyrimidin-5-yl)-N-(2,2-difluoroethyl)imidazo [1,2-a] pyridine-3-carboxamide [410];
N-(2,2-difluoroethyl)-6-(2-(methylamino)-7H-pyrrolo [2,3-d] pyrimidin-5-yl)imidazo [1,2-a] pyridine-3-carboxamide [411];
N-(2,2-difluoroethyl)-6-(2-(ethylamino)-7H-pyrrolo [2,3-d] pyrimidin-5-yl)imidazo [1,2-a] pyridine-3-carboxamide [412];
N-(2,2-difluoroethyl)-6-(2-(i sop ropylamino)-7H-pyrrolo [2,3-d]pyrimidin-5-yl)imidazo [1,2-a] pyridine-3-carboxamide [413];
N-(2,2-difluoroethyl)-6-(2-(isobutylamino)-7H-pyrrolo [2,3-d]pyrimidin-5-yl)imidazo [1,2-a] pyridine-3-carboxamide [414];
N-(2,2-difluoroethyl)-6-(2-(neopentylamino)-7H-pyrrolo [2,3-d]pyrimidin-5-y0imidazo [1,2-a] pyridine-3-carboxamide [415];
(S)-6-(2-(sec-butylamino)-7H-pyrrolo [2,3-d] pyrimidin-5-yl)-N-(2,2-difluoroethyl)imidazo [1,2-a] pyridine-3-carboxamide [416];
N-(2,2-difluoroethyl)-6-(2-((2-fluoro-2-methylpropyl)amino)-7H-pyrrolo [2,3-d]pyrimidin-5-yl)imidazo[1,2-a]pyridine-3-carboxamide [417];
N-(2,2-difluoroethyl)-6-(2-((2,2-difluoropropyl)amino)-7H-pyrrolo [2,3-d]pyrimidin-5-yl)imidazo[1,2-a]pyridine-3-carboxamide [418];
N-(2,2-difluoroethyl)-6-(2-((2,2,2-trifluoroethyl)amino)-7H-pyrrolo[2,3-d]pyrimidin-5-yl)imidazo[1,2-a]pyridine-3-carboxamide [419];
(R)-N-(2,2-difluoroethyl)-6-(2-((1,1,1-trifluoropropan-2-yl)amino)-7H-pyrrolo[2,3-d]pyrimidin-5-yl)imidazo[1,2-a]pyridine-3-carboxamide [420];
(S)-N-(2,2-difluoroethyl)-6-(2-((1,1,1-trifluoropropan-2-yl)amino)-7H-pyrrolo[2,3-d]pyrimidin-5-yl)imidazo[1,2-a]pyridine-3-carboxamide [421];
N-(2,2-difluoroethyl)-6-(2-((3,3,3-trifluoropropyl)amino)-7H-pyrrolo[2,3-d]pyrimidin-5-yl)imidazo[1,2-a]pyridine-3-carboxamide [422];
(S)-N-(2,2-difluoroethyl)-6-(2-((l-methoxypropan-2-yl)amino)-7H-pyrrolo[2,3-d]pyrimidin-5-yl)imidazo[1,2-a]pyridine-3-carboxamide [423];
N-(2,2-difluoroethyl)-6-(2-ethoxy-7H-pyrrolo[2,3-d]pyrimidin-5-yl)imidazo[1,2-a]pyridine-3-carboxamide [424];
N-(2,2-difluoroethyl)-6-(2-(((1-fluorocyclopropyl)methyl)amino)-7H-pyrrolo[2,3-d]pyrimidin-5-yl)imidazo[1,2-a]pyridine-3-carboxamide [425];
(R)-6-(2-((l-cyclopropylethyl)amino)-7H-pyrrolo[2,3-d]pyrimidin-5-yl)-N-(2,2-difluoroethyl)imidazo[1,2-a]pyridine-3-carboxamide [426];
N-(2,2-difluoroethyl)-6-(2-(((l-methylcyclopropyl)methyl)amino)-7H-pyrrolo[2,3-d]pyrimidin-5-yl)imidazo[1,2-a]pyridine-3-carboxamide [427];
N-(2,2-difluoroethyl)-6-(2-(((1-(trifluoromethyl)cyclopropyl)methyl)amino)-7H-pyrrolo[2,3-d]pyrimidin-5-yl)imidazo[1,2-a]pyridine-3-carboxamide [428];
6-(2-((2-cyclopropyl-2,2-difluoroethyl)amino)-7H-pyrrolo[2,3-d]pyrimidin-5-yl)-N-(2,2-difluoroethyl)imidazo[1,2-a]pyridine-3-carboxamide [429];
6-(2-((3,3-difluorocyclobutyl)amino)-7H-pyrrolo[2,3-d]pyrimidin-5-yl)-N-(2,2-difluoroethyl)imidazo[1,2-a]pyridine-3-carboxamide [430];
N-(2,2-difluoroethyl)-6-(2-(((1-fluorocyclobutyl)methyl)amino)-7H-pyrrolo[2,3-d]pyrimidin-5-yl)imidazo[1,2-a]pyridine-3-carboxamide [431];
6-(2-((4,4-difluorocyclohexyl)amino)-7H-pyrrolo[2,3-d]pyrimidin-5-yl)-N-(2,2-difluoroethyl)imidazo[1,2-a]pyridine-3-carboxamide [432];
N-(2,2-difluoroethyl)-6-(2-((cis-4-methoxycyclohexyl)amino)-7H-pyrrolo[2,3-d]pyrimidin-5-yl)imidazo[1,2-a]pyridine-3-carboxamide [433];
N-(2,2-difluoroethyl)-6-(2-(((1-fluorocyclohexyl)methyl)amino)-7H-pyrrolo[2,3-d]pyrimidin-5-yl)imidazo[1,2-a]pyridine-3-carboxamide [434];
6-(2-(((4,4-difluorocyclohexyl)methyl)amino)-7H-pyrrolo[2,3-d]pyrimidin-5-yl)-N-(2,2-difluoroethyl)imidazo[1,2-a]pyridine-3-carboxamide [435];
N-(2,2-difluoroethyl)-6-(2-((l-methylpiperidin-4-yl)amino)-7H-pyrrolo[2,3-d]pyrimidin-5-yl)imidazo[1,2-a]pyridine-3-carboxamide [436];
N-(2,2-difluoroethyl)-6-(2-(((l-methylpiperidin-4-yl)methyl)amino)-7H-pyrrolo[2,3-d]pyrimidin-5-yl)imidazo[1,2-a]pyridine-3-carboxamide [437];
N-(2,2-difluoroethyl)-6-(2-((tetrahydro-2H-pyran-4-yl)amino)-7H-pyrrolo[2,3-d]pyrimidin-5-yl)imidazo[1,2-a]pyridine-3-carboxamide [438];
N-(2,2-difluoroethyl)-6-(2-((3-(4-methylpiperazin-1-yl)phenyl)amino)-7H-pyrrolo[2,3-d]pyrimidin-5-yl)imidazo[1,2-a]pyridine-3-carboxamide [439];
N-(2,2-difluoroethyl)-6-(2-(pyridin-4-ylamino)-7H-pyrrolo[2,3-d]pyrimidin-5-yl)imidazo[1,2-a]pyridine-3-carboxamide [440];
N-(2,2-difluoroethyl)-6-(2-((2-(4-methylpiperazin-1-yl)pyridin-4-yl)amino)-7H-pyrrolo[2,3-d]pyrimidin-5-yl)imidazo[1,2-a]pyridine-3-carboxamide [441];
N-(2,2-difluoroethyl)-6-(2-((6-(4-methylpiperazin-1-yl)pyridin-3-yl)amino)-7H-pyrrolo[2,3-d]pyrimidin-5-yl)imidazo[1,2-a]pyridine-3-carboxamide [442];
N-(2,2-difluoroethyl)-6-(2-(46-(4-methylpiperazin-1-yl)pyridin-3-yl)methyl)amino)-7H-pyrrolo[2,3-d]pyrimidin-5-yl)imidazo[1,2-a]pyridine-3-carboxamide [443];
N-(2,2-difluoroethyl)-6-(2-(quinolin-6-yl)-7H-pyrrolo[2,3-d]pyrimidin-5-yl)imidazo[1,2-a]pyridine-3-carboxamide [444];
N-(2,2-difluoroethyl)-6-(4-(1-methyl-1H-pyrazol-4-yl)-7H-pyrrolo[2,3-d]pyrimidin-5-yl)imidazo[1,2-a]pyridine-3-carboxamide [445];
N-(2,2-difluoroethyl)-6-(4-((trans-4-morpholinocyclohexyl)amino)-7H-pyrrolo[2,3-d]pyrimidin-5-yl)imidazo[1,2-a]pyridine-3-carboxamide [446];
5-(imidazo[1,2-a]pyrimidin-6-yl)-2-isobutyl-7H-pyrrolo[2,3-d]pyrimidine [448];
5-(imidazo[1,2-a]pyrimidin-6-yl)-2-(3,3,3-trifluoropropyl)-7H-pyrrolo[2,3-d]pyrimidine [449];
2-cyclopropyl-5-(imidazo[1,2-a]pyrimidin-6-yl)-7H-pyrrolo[2,3-d]pyrimidine [450];
2-cyclobutyl-5-(imidazo[1,2-a]pyrimidin-6-yl)-7H-pyrrolo[2,3-d]pyrimidine [451];
2-(cyclobutylmethyl)-5-(imidazo[1,2-a]pyrimidin-6-yl)-7H-pyrrolo[2,3-d]pyrimidine [452];
5-(imidazo[1,2-a]pyrimidin-6-yl)-7H-pyrrolo[2,3-d]pyrimidin-2-amine [453];
5-(imidazo[1,2-a]pyrimidin-6-yl)-N-methyl-7H-pyrrolo[2,3-d]pyrimidin-2-amine [454];
N-ethyl-5-(imidazo[1,2-a]pyrimidin-6-yl)-7H-pyrrolo[2,3-d]pyrimidin-2-amine [455];
5-(imidazo[1,2-a]pyrimidin-6-yl)-N-isopropyl-7H-pyrrolo[2,3-d]pyrimidin-2-amine [456];
5-(imidazo[1,2-a]pyrimidin-6-yl)-N-isobutyl-7H-pyrrolo[2,3-d]pyrimidin-2-amine [457];
5-(imidazo[1,2-a]pyrimidin-6-yl)-N-neopentyl-7H-pyrrolo[2,3-d]pyrimidin-2-amine [458];
(S)-N-(sec-butyl)-5-(imidazo[1,2-a]pyrimidin-6-yl)-7H-pyrrolo[2,3-d]pyrimidin-2-amine [459];
N-(2-fluoro-2-methylpropyl)-5-(imidazo[1,2-a]pyrimidin-6-yl)-7H-pyrrolo[2,3-d]pyrimidin-2-amine [460];
N-(2,2-difluoropropyl)-5-(imidazo[1,2-a]pyrimidin-6-yl)-7H-pyrrolo[2,3-d]pyrimidin-2-amine [461];
5-(imidazo[1,2-a]pyrimidin-6-yl)-N-(2,2,2-trifluoroethyl)-7H-pyrrolo[2,3-d]pyrimidin-2-amine
[462];
(R)-5-(imidazo[1,2-a]pyrimidin-6-yl)-N-(1,1,1-trifluoropropan-2-yl)-7H-pyrrolo[2,3-d]pyrimidin-2-amine [463];
(S)-5-(imidazo[1,2-a]pyrimidin-6-yl)-N-(1,1,1-trifluoropropan-2-yl)-7H-pyrrolo[2,3-d]pyrimidin-2-amine [464];
5-(imidazo[1,2-a]pyrimidin-6-yl)-N-(3,3,3-trifluoropropyl)-7H-pyrrolo[2,3-d]pyrimidin-2-amine [465];
(S)-5-(imidazo[1,2-a]pyrimidin-6-yl)-N-(1-methoxypropan-2-yl)-7H-pyrrolo[2,3-d]pyrimidin-2-amine
[466];
2-ethoxy-5-(imidazo[1,2-a]pyrimidin-6-yl)-7H-pyrrolo[2,3-d]pyrimidine [467];
N-((1-fluorocyclopropyl)methyl)-5-(imidazo[1,2-a]pyrimidin-6-yl)-7H-pyrrolo[2,3-d]pyrimidin-2-amine [468];
(R)-N-(1-cyclopropylethyl)-5-(imidazo[1,2-a]pyrimidin-6-yl)-7H-pyrrolo[2,3-d]pyrimidin-2-amine [469];
5-(imidazo[1,2-a]pyrimidin-6-yl)-N-((1-methylcyclopropyl)methyl)-7H-pyrrolo[2,3-d]pyrimidin-2-amine [470];
5-(imidazo[1,2-a]pyrimidin-6-yl)-N-41-(trifluoromethyl)cyclopropyl)methyl)-7H-pyrrolo[2,3-d]pyrimidin-2-amine [471];
N-(2-cyclopropyl-2,2-difluoroethyl)-5-(imidazo[1,2-a]pyrimidin-6-yl)-7H-pyrrolo[2,3-d]pyrimidin-2-amine [472];
N-(3,3-difluorocyclobutyl)-5-(imidazo[1,2-a]pyrimidin-6-yl)-7H-pyrrolo[2,3-d]pyrimidin-2-amine [473];
N-((1-fluorocyclobutyl)methyl)-5-(imidazo[1,2-a]pyrimidin-6-yl)-7H-pyrrolo[2,3-d]pyrimidin-2-amine [474];
N-(4,4-difluorocyclohexyl)-5-(imidazo[1,2-a]pyrimidin-6-yl)-7H-pyrrolo[2,3-d]pyrimidin-2-amine [475];
5-(imidazo[1,2-a]pyrimidin-6-yl)-N-(cis-4-methoxycyclohexyl)-7H-pyrrolo[2,3-d]pyrimidin-2-amine [476];
N-((1-fluorocyclohexyl)methyl)-5-(imidazo[1,2-a]pyrimidin-6-yl)-7H-pyrrolo[2,3-d]pyrimidin-2-amine [477];
N-((4,4-difluorocyclohexyl)methyl)-5-(imidazo[1,2-a]pyrimidin-6-yl)-7H-pyrrolo[2,3-d]pyrimidin-2-amine [478];
5-(imidazo[1,2-a]pyrimidin-6-yl)-N-(1-methylpiperidin-4-yl)-7H-pyrrolo[2,3-d]pyrimidin-2-amine [479];
5-(imidazo[1,2-a]pyrimidin-6-yl)-N-((1-methylpiperidin-4-yl)methyl)-7H-pyrrolo[2,3-d]pyrimidin-2-amine [480];
5-(imidazo[1,2-a]pyrimidin-6-yl)-N-(tetrahydro-2H-pyran-4-yl)-7H-pyrrolo[2,3-d]pyrimidin-2-amine [481];
5-(imidazo[1,2-a]pyrimidin-6-yl)-N-(3-(4-methylpiperazin-1-yl)phenyl)-7H-pyrrolo[2,3-d]pyrimidin-2-amine [482];
5-(imidazo[1,2-a]pyrimidin-6-yl)-N-(pyridin-4-yl)-7H-pyrrolo[2,3-d]pyrimidin-2-amine [483];
5-(imidazo[1,2-a]pyrimidin-6-yl)-N-(2-(4-methylpiperazin-1-yl)pyridin-4-yl)-7H-pyrrolo[2,3-d]pyrimidin-2-amine [484];
5-(imidazo[1,2-a]pyrimidin-6-yl)-N-(6-(4-methylpiperazin-1-yl)pyridin-3-yl)-7H-pyrrolo[2,3-d]pyrimidin-2-amine [485];
5-(imidazo[1,2-a]pyrimidin-6-yl)-N-((6-(4-methylpiperazin-1-yl)pyridin-3-yl)methyl)-7H-pyrrolo[2,3-d]pyrimidin-2-amine [486];
6-(5-(imidazo[1,2-a]pyrimidin-6-yl)-7H-pyrrolo[2,3-d]pyrimidin-2-yl)quinoline [487];
5-(imidazo[1,2-a]pyrimidin-6-yl)-4-(1-methyl-1H-pyrazol-4-yl)-7H-pyrrolo[2,3-d]pyrimidine [488];
5-(imidazo[1,2-a]pyrimidin-6-yl)-N-(trans-4-morpholinocyclohexyl)-7H-pyrrolo[2,3-d]pyrimidin-4-amine [489];
5-(imidazo[1,2-a]pyrimidin-6-yl)-2-neopentyl-7H-pyrrolo[2,3-d]pyrimidine [490];
N-(2-fluoro-2-methylpropyl)-5-(2-methyl-2H-indazol-5-yl)-7H-pyrrolo[2,3-d]pyrimidin-2-amine
[492];
5-(4-fluoro-1,2-dimethyl-1H-benzo[d]imidazol-6-yl)-N-((1-methylazetidin-3-yl)methyl)-7H-pyrrolo[2,3-d]pyrimidin-2-amine [493];
2-(azetidin-1-yl)-5-(4-fluoro-1-isopropyl-2-methyl-1H-benzo[d]imidazol-6-yl)-7H-pyrrolo[2,3-d]pyrimidine [494];
5-(4-fluoro-1-isopropyl-2-methyl-1H-benzo[d]imidazol-6-yl)-2-isobutyl-7H-pyrrolo[2,3-d]pyrimidine [496];
5-(4-fluoro-1-isopropyl-2-methyl-1H-benzo[d]imidazol-6-yl)-2-isobutyl-4-methoxy-7H-pyrrolo[2,3-d]pyrimidine [497];
5-(4-fluoro-1-isopropyl-2-methyl-1H-benzo[d]imidazol-6-yl)-2-(3,3,3-trifluoropropyl)-7H-pyrrolo[2,3-d]pyrimidine [498];
2-cyclopropyl-5-(4-fluoro-1-isopropyl-2-methyl-1H-benzo [d]imidazol-6-yl)-7H-pyrrolo [2,3-d]pyrimidine [499]; and
2-cyclobutyl-5-(4-fluoro-1-isopropyl-2-methyl-1H-benzo [d]imidazol-6-yl)-7H-pyrrolo [2,3-d]pyrimidine [500]; or a pharmaceutically acceptable salt thereof

24. The compound of claim 1, wherein the compound of Formula I is selected from the group consisting of:
2-(cyclobutylmethyl)-5-(4-fluoro-1-isopropyl-2-methyl-1H-benzo[d]imidazol-6-yl)-7H-pyrrolo[2,3-d]pyrimidine [501];
5-(4-fluoro-1-isopropyl-2-methyl-1H-benzo [d]imidazol-6-yl)-7H-pyrrolo [2,3-d]pyrimidin-2-amine [502];
5-(4-fluoro-1-isopropyl-2-methyl-1H-benzo [d]imidazol-6-yl)-N-methyl-7H-pyrrolo [2,3-d]pyrimidin-2-amine [503];
N-ethyl-5-(4-fluoro-1-isopropyl-2-methyl-1H-benzo [d]imidazol-6-yl)-7H-pyrrolo [2,3-d]pyrimidin-2-amine [504];
5-(4-fluoro-1-isopropyl-2-methyl-1H-benzo [d]imidazol-6-yl)-N-isopropyl-7H-pyrrolo [2,3-d]pyrimidin-2-amine [505];
5-(4-fluoro-1-isopropyl-2-methyl-1H-benzo [d]imidazol-6-yl)-N-isobutyl-7H-pyrrolo [2,3-d]pyrimidin-2-amine [506];
5-(4-fluoro-1-isopropyl-2-methyl-1H-benzo [d]imidazol-6-yl)-N-neopentyl-7H-pyrrolo [2,3-d]pyrimidin-2-amine [507];
(S)-N-(sec-butyl)-5-(4-fluoro-1-isopropyl-2-methyl-1H-benzo [d]imidazol-6-yl)-7H-pyrrolo [2,3-d]pyrimidin-2-amine [508];
5-(4-fluoro-1-isopropyl-2-methyl-1H-benzo[d]imidazol-6-yl)-N-(2-fluoro-2-methylpropyl)-7H-pyrrolo[2,3-d]pyrimidin-2-amine [509];
N-(2,2-difluoropropyl)-5-(4-fluoro-1-isopropyl-2-methyl-lH-benzo[d]imidazol-6-yl)-7H-pyrrolo[2,3-d]pyrimidin-2-amine [510];
5-(4-fluoro-1-isopropyl-2-methyl-1H-benzo[d]imidazol-6-yl)-N-(2,2,2-trifluoroethyl)-7H-pyrrolo[2,3-d]pyrimidin-2-amine [511];
(R)-5-(4-fluoro-1-isopropyl-2-methyl-1H-benzo[d]imidazol-6-yl)-N-(1,1,1-trifluoropropan-2-yl)-7H-pyrrolo[2,3-d]pyrimidin-2-amine [512];
(S)-5-(4-fluoro-1-isopropyl-2-methyl-1H-benzo[d]imidazol-6-yl)-N-(1,1,1-trifluoropropan-2-yl)-7H-pyrrolo[2,3-d]pyrimidin-2-amine [513];
5-(4-fluoro-1-isopropyl-2-methyl-1H-benzo[d]imidazol-6-yl)-N-(3,3,3-trifluoropropyl)-7H-pyrrolo[2,3-d]pyrimidin-2-amine [514];
(S)-5-(4-fluoro-1-isopropyl-2-methyl-1H-benzo[d]imidazol-6-yl)-N-(1-methoxypropan-2-yl)-7H-pyrrolo[2,3-d]pyrimidin-2-amine [515];
2-ethoxy-5-(4-fluoro-1-isopropyl-2-methyl-1H-benzo[d]imidazol-6-yl)-7H-pyrrolo[2,3-d]pyrimidine [516];
5-(4-fluoro-1-isopropyl-2-methyl-1H-benzo[d]imidazol-6-yl)-N-((1-fluorocyclopropyl)methyl)-7H-pyrrolo[2,3-d]pyrimidin-2-amine [517];
(R)-N-(1-cyclopropylethyl)-5-(4-fluoro-1-isopropyl-2-methyl-1H-benzo[d]imidazol-6-yl)-7H-pyrrolo[2,3-d]pyrimidin-2-amine [518];
5-(4-fluoro-1-isopropyl-2-methyl-1H-benzo[d]imidazol-6-yl)-N-((1-methylcyclopropyl)methyl)-7H-pyrrolo[2,3-d]pyrimidin-2-amine [519];
5-(4-fluoro-1-isopropyl-2-methyl-1H-benzo[d]imidazol-6-yl)-N-41-(trifluoromethyl)cyclopropyl)methyl)-7H-pyrrolo[2,3-d]pyrimidin-2-amine [520];
N-(2-cyclopropyl-2,2-difluoroethyl)-5-(4-fluoro-1-isopropyl-2-methyl-1H-benzo[d]imidazol-6-yl)-7H-pyrrolo[2,3-d]pyrimidin-2-amine [521];
N-(3,3-difluorocyclobutyl)-5-(4-fluoro-1-isopropyl-2-methyl-1H-benzo[d]imidazol-6-yl)-7H-pyrrolo[2,3-d]pyrimidin-2-amine [522];
5-(4-fluoro-1-isopropyl-2-methyl-1H-benzo[d]imidazol-6-yl)-N-((1-fluorocyclobutyl)methyl)-7H-pyrrolo[2,3-d]pyrimidin-2-amine [523];
N-(4,4-difluorocyclohexyl)-5-(4-fluoro-1-isopropyl-2-methyl-1H-benzo[d]imidazol-6-yl)-7H-pyrrolo[2,3-d]pyrimidin-2-amine [524];
5-(4-fluoro-1-isopropyl-2-methyl-1H-benzo[d]imidazol-6-yl)-N-(cis-4-methoxycyclohexyl)-7H-pyrrolo[2,3-d]pyrimidin-2-amine [525];
5-(4-fluoro-1-isopropyl-2-methyl-1H-benzo[d]imidazol-6-yl)-N-((1-fluorocyclohexyl)methyl)-7H-pyrrolo[2,3-d]pyrimidin-2-amine [526];
N-((4,4-difluorocyclohexyl)methyl)-5-(4-fluoro-1-isopropyl-2-methyl-1H-benzo[d]imidazol-6-yl)-7H-pyrrolo[2,3-d]pyrimidin-2-amine [527];
5-(4-fluoro-1-isopropyl-2-methyl-1H-benzo[d]imidazol-6-yl)-N-(1-methylpiperidin-4-yl)-7H-pyrrolo[2,3-d]pyrimidin-2-amine [528];
5-(4-fluoro-1-isopropyl-2-methyl-1H-benzo[d]imidazol-6-yl)-N-((1-methylpiperidin-4-yl)methyl)-7H-pyrrolo[2,3-d]pyrimidin-2-amine [529];
5-(4-fluoro-1-isopropyl-2-methyl-1H-benzo[d]imidazol-6-yl)-N-(tetrahydro-2H-pyran-4-yl)-7H-pyrrolo[2,3-d]pyrimidin-2-amine [530];
5-(4-fluoro-1-isopropyl-2-methyl-1H-benzo[d]imidazol-6-yl)-N-(3-(4-methylpiperazin-1-yl)phenyl)-7H-pyrrolo[2,3-d]pyrimidin-2-amine [531];
5-(4-fluoro-1-isopropyl-2-methyl-1H-benzo[d]imidazol-6-yl)-N-(pyridin-4-yl)-7H-pyrrolo[2,3-d]pyrimidin-2-amine [532];
5-(4-fluoro-1-isopropyl-2-methyl-1H-benzo[d]imidazol-6-yl)-N-(2-(4-methylpiperazin-1-yl)pyridin-4-yl)-7H-pyrrolo[2,3-d]pyrimidin-2-amine [533];
5-(4-fluoro-1-isopropyl-2-methyl-1H-benzo[d]imidazol-6-yl)-N-(6-(4-methylpiperazin-1-yl)pyridin-3-yl)-7H-pyrrolo[2,3-d]pyrimidin-2-amine [534];
5-(4-fluoro-1-isopropyl-2-methyl-1H-benzo[d]imidazol-6-yl)-N-46-(4-methylpiperazin-1-yl)pyridin-3-yl)methyl)-7H-pyrrolo[2,3-d]pyrimidin-2-amine [535];
6-(5-(4-fluoro-1-isopropyl-2-methyl-1H-benzo[d]imidazol-6-yl)-7H-pyrrolo[2,3-d]pyrimidin-2-yl)quinoline [536];
5-(4-fluoro-1-isopropyl-2-methyl-1H-benzo[d]imidazol-6-yl)-4-(1-methyl-1H-pyrazol-4-yl)-7H-pyrrolo[2,3-d]pyrimidine [537];
5-(4-fluoro-1-isopropyl-2-methyl-1H-benzo[d]imidazol-6-yl)-N-(trans-4-morpholinocyclohexyl)-7H-pyrrolo[2,3-d]pyrimidin-4-amine [538];
2-cyclopropyl-5-(5-fluoro-1-isopropyl-2-methyl-1H-benzo[d]imidazol-6-yl)-7H-pyrrolo[2,3-d]pyrimidine [539];
2-cyclopropyl-5-(7-fluoro-1-isopropyl-2-methyl-1H-benzo[d]imidazol-6-yl)-7H-pyrrolo[2,3-d]pyrimidine [540];
5-(4-fluoro-2-methyl-1-(1-methylpiperidin-4-yl)-1H-benzo[d]imidazol-6-yl)-2-isobutyl-7H-pyrrolo[2,3-d]pyrimidine [542];
5-(4-fluoro-2-methyl-1-(1-methylpiperidin-4-yl)-1H-benzo[d]imidazol-6-yl)-2-(3,3,3-trifluoropropyl)-7H-pyrrolo[2,3-d]pyrimidine [543];
2-cyclopropyl-5-(4-fluoro-2-methyl-1-(1-methylpiperidin-4-yl)-1H-benzo[d]imidazol-6-yl)-7H-pyrrolo[2,3-d]pyrimidine [544];
2-cyclobutyl-5-(4-fluoro-2-methyl-1-(1-methylpiperidin-4-yl)-1H-benzo[d]imidazol-6-yl)-7H-pyrrolo[2,3-d]pyrimidine [545];
2-(cyclobutylmethyl)-5-(4-fluoro-2-methyl-1-(1-methylpiperidin-4-yl)-1H-benzo[d]imidazol-6-yl)-7H-pyrrolo[2,3-d]pyrimidine [546];
5-(4-fluoro-2-methyl-1-(1-methylpiperidin-4-yl)-1H-benzo[d]imidazol-6-yl)-7H-pyrrolo[2,3-d]pyrimidin-2-amine [547];
5-(4-fluoro-2-methyl-1-(1-methylpiperidin-4-yl)-1H-benzo[d]imidazol-6-yl)-N-methyl-7H-pyrrolo[2,3-d]pyrimidin-2-amine [548];
N-ethyl-5-(4-fluoro-2-methyl-1-(1-methylpiperidin-4-yl)-1H-benzo[d]imidazol-6-yl)-7H-pyrrolo[2,3-d]pyrimidin-2-amine [549];
5-(4-fluoro-2-methyl-1-(1-methylpiperidin-4-yl)-1H-benzo[d]imidazol-6-yl)-N-isopropyl-7H-pyrrolo[2,3-d]pyrimidin-2-amine [550];
5-(4-fluoro-2-methyl-1-(1-methylpiperidin-4-yl)-1H-benzo[d]imidazol-6-yl)-N-isobutyl-7H-pyrrolo[2,3-d]pyrimidin-2-amine [551];
5-(4-fluoro-2-methyl-1-(1-methylpiperidin-4-yl)-1H-benzo[d]imidazol-6-yl)-N-neopentyl-7H-pyrrolo[2,3-d]pyrimidin-2-amine [552];
(S)-N-(sec-butyl)-5-(4-fluoro-2-methyl-1-(1-methylpiperidin-4-yl)-1H-benzo[d]imidazol-6-yl)-7H-pyrrolo[2,3-d]pyrimidin-2-amine [553];
5-(4-fluoro-2-methyl-1-(1-methylpiperidin-4-yl)-1H-benzo[d]imidazol-6-yl)-N-(2-fluoro-2-methylpropyl)-7H-pyrrolo[2,3-d]pyrimidin-2-amine [554];
N-(2,2-difluoropropyl)-5-(4-fluoro-2-methyl-1-(1-methylpiperidin-4-yl)-1H-benzo[d]imidazol-6-yl)-7H-pyrrolo[2,3-d]pyrimidin-2-amine [555];
5-(4-fluoro-2-methyl-1-(1-methylpiperidin-4-yl)-1H-benzo[d]imidazol-6-yl)-N-(2,2,2-trifluoroethyl)-7H-pyrrolo[2,3-d]pyrimidin-2-amine [556];
(R)-5-(4-fluoro-2-methyl-1-(1-methylpiperidin-4-yl)-1H-benzo[d]imidazol-6-yl)-N-(1,1,1-trifluoropropan-2-yl)-7H-pyrrolo[2,3-d]pyrimidin-2-amine [557];
(S)-5-(4-fluoro-2-methyl-1-(1-methylpiperidin-4-yl)-1H-benzo[d]imidazol-6-yl)-N-(1,1,1-trifluoropropan-2-yl)-7H-pyrrolo[2,3-d]pyrimidin-2-amine [558];
5-(4-fluoro-2-methyl-1-(1-methylpiperidin-4-yl)-1H-benzo[d]imidazol-6-yl)-N-(3,3,3-trifluoropropyl)-7H-pyrrolo[2,3-d]pyrimidin-2-amine [559];
(S)-5-(4-fluoro-2-methyl-1-(1-methylpiperidin-4-yl)-1H-benzo[d]imidazol-6-yl)-N-(1-methoxypropan-2-yl)-7H-pyrrolo[2,3-d]pyrimidin-2-amine [560];
2-ethoxy-5-(4-fluoro-2-methyl-1-(1-methylpiperidin-4-yl)-1H-benzo[d]imidazol-6-yl)-7H-pyrrolo[2,3-d]pyrimidine [561];
5-(4-fluoro-2-methyl-1-(1-methylpiperidin-4-yl)-1H-benzo[d]imidazol-6-yl)-N-((1-fluorocyclopropyl)methyl)-7H-pyrrolo[2,3-d]pyrimidin-2-amine [562];
(R)-N-(1-cyclopropylethyl)-5-(4-fluoro-2-methyl-1-(1-methylpiperidin-4-yl)-1H-benzo[d]imidazol-6-yl)-7H-pyrrolo[2,3-d]pyrimidin-2-amine [563];
5-(4-fluoro-2-methyl-1-(1-methylpiperidin-4-yl)-1H-benzo[d]imidazol-6-yl)-N-((1-methylcyclopropyl)methyl)-7H-pyrrolo[2,3-d]pyrimidin-2-amine [564];
5-(4-fluoro-2-methyl-1-(1-methylpiperidin-4-yl)-1H-benzo[d]imidazol-6-yl)-N-41-(trifluoromethyl)cyclopropyl)methyl)-7H-pyrrolo[2,3-d]pyrimidin-2-amine [565];
N-(2-cyclopropyl-2,2-difluoroethyl)-5-(4-fluoro-2-methyl-1-(1-methylpiperidin-4-yl)-1H-benzo[d]imidazol-6-yl)-7H-pyrrolo[2,3-d]pyrimidin-2-amine [566];
N-(3,3-difluorocyclobutyl)-5-(4-fluoro-2-methyl-1-(1-methylpiperidin-4-yl)-1H-benzo[d]imidazol-6-yl)-7H-pyrrolo[2,3-d]pyrimidin-2-amine [567];
5-(4-fluoro-2-methyl-1-(1-methylpiperidin-4-yl)-1H-benzo[d]imidazol-6-yl)-N-((1-fluorocyclobutyl)methyl)-7H-pyrrolo[2,3-d]pyrimidin-2-amine [568];
N-(4,4-difluorocyclohexyl)-5-(4-fluoro-2-methyl-1-(1-methylpiperidin-4-yl)-1H-benzo[d]imidazol-6-yl)-7H-pyrrolo[2,3-d]pyrimidin-2-amine [569];
5-(4-fluoro-2-methyl-1-(1-methylpiperidin-4-yl)-1H-benzo[d]imidazol-6-yl)-N-(cis-4-methoxycyclohexyl)-7H-pyrrolo[2,3-d]pyrimidin-2-amine [570];
5-(4-fluoro-2-methyl-1-(1-methylpiperidin-4-yl)-1H-benzo[d]imidazol-6-yl)-N-((1-fluorocyclohexyl)methyl)-7H-pyrrolo[2,3-d]pyrimidin-2-amine [571];
N-((4,4-difluorocyclohexyl)methyl)-5-(4-fluoro-2-methyl-1-(1-methylpiperidin-4-yl)-1H-benzo[d]imidazol-6-yl)-7H-pyrrolo[2,3-d]pyrimidin-2-amine [572];
5-(4-fluoro-2-methyl-1-(1-methylpiperidin-4-yl)-1H-benzo[d]imidazol-6-yl)-N-(1-methylpiperidin-4-yl)-7H-pyrrolo[2,3-d]pyrimidin-2-amine [573];
5-(4-fluoro-2-methyl-1-(1-methylpiperidin-4-yl)-1H-benzo[d]imidazol-6-yl)-N-((1-methylpiperidin-4-yl)methyl)-7H-pyrrolo[2,3-d]pyrimidin-2-amine [574];
5-(4-fluoro-2-methyl-1-(1-methylpiperidin-4-yl)-1H-benzo[d]imidazol-6-yl)-N-(tetrahydro-2H-pyran-4-yl)-7H-pyrrolo[2,3-d]pyrimidin-2-amine [575];
5-(4-fluoro-2-methyl-1-(1-methylpiperidin-4-yl)-1H-benzo[d]imidazol-6-yl)-N-(3-(4-methylpiperazin-1-yl)phenyl)-7H-pyrrolo[2,3-d]pyrimidin-2-amine [576];
5-(4-fluoro-2-methyl-1-(1-methylpiperidin-4-yl)-1H-benzo[d]imidazol-6-yl)-N-(pyridin-4-yl)-7H-pyrrolo[2,3-d]pyrimidin-2-amine [577];
5-(4-fluoro-2-methyl-1-(1-methylpiperidin-4-yl)-1H-benzo[d]imidazol-6-yl)-N-(2-(4-methylpiperazin-1-yppyridin-4-yl)-7H-pyrrolo[2,3-d]pyrimidin-2-amine [578];
5-(4-fluoro-2-methyl-1-(1-methylpiperidin-4-yl)-1H-benzo[d]imidazol-6-yl)-N-(6-(4-methylpiperazin-1-yppyridin-3-yl)-7H-pyrrolo[2,3-d]pyrimidin-2-amine [579];
5-(4-fluoro-2-methyl-1-(1-methylpiperidin-4-yl)-1H-benzo[d]imidazol-6-yl)-N-46-(4-methylpiperazin-1-yl)pyridin-3-yl)methyl)-7H-pyrrolo[2,3-d]pyrimidin-2-amine [580];
6-(5-(4-fluoro-2-methyl-1-(1-methylpiperidin-4-yl)-1H-benzo [d] imidazol-6-yl)-7H-pyrrolo [2,3-d]pyrimidin-2-yl)quinoline [581];
5-(4-fluoro-2-methyl-1-(1-methylpiperidin-4-yl)-1H-benzo [d]imidazol-6-yl)-4-(1-methyl-1H-pyrazol-4-yl)-7H-pyrrolo [2,3-d] pyrimidine [582];
5-(4-fluoro-2-methyl-1-(1-methylpiperidin-4-yl)-1H-benzo [d] imidazol-6-yl)-N-(trans-4-morpholinocyclohexyl)-7H-pyrrolo [2,3-d] pyrimidin-4-amine [583];
6-(2-isobutyl-7H-pyrrolo [2,3-d] pyrimidin-5-yl)-1-isopropyl-2-methyl-1H-imidazo [4,5-b] pyridine [585];
1-i sopropyl-2-methyl-6-(2-(3,3,3-trifluoropropyl)-7H-pyrrolo [2,3-d]pyrimidin-5-yl)-1H-imidazo[4,5-b]pyridine [586];
6-(2-cyclopropyl-7H-pyrrolo [2,3-d]pyrimidin-5-yl)-1-isopropyl-2-methyl-1H-imidazo [4,5-b]pyridine [587];
6-(2-cyclobutyl-7H-pyrrolo [2,3-d]pyrimidin-5-yl)-1-i sopropyl-2-methyl-1H-imidazo [4,5-b]pyridine [588];
6-(2-(cyclobutylmethyl)-7H-pyrrolo [2,3-d]pyrimidin-5-yl)-1-isopropyl-2-methyl-1H-imidazo[4,5-b]pyridine [589];
5-(1-i sopropyl-2-methyl-1H-imidazo [4,5-b]pyridin-6-yl)-7H-pyrrolo [2,3-d] pyrimidin-2-amine [590];
5-(1-i sopropyl-2-methyl-1H-imidazo [4,5-b]pyridin-6-yl)-N-methyl-7H-pyrrolo [2,3-d]pyrimidin-2-amine [591];
N-ethyl-5-(1-i sop ropyl-2-methyl-1H-imidazo [4,5-b]pyridin-6-yl)-7H-pyrrolo [2,3-d] pyrimidin-2-amine [592];
N-isopropyl-5-(1-isopropyl-2-methyl-1H-imidazo [4,5-b]pyridin-6-yl)-7H-pyrrolo [2,3-d]pyrimidin-2-amine [593];
N-i sobutyl-5-(1-i sop ropyl-2-methyl-1H-imidazo [4,5-b]pyridin-6-yl)-7H-pyrrolo [2,3-d] pyrimidin-2-amine [594];
5-(1-i sopropyl-2-methyl-1H-imidazo [4,5-b]pyridin-6-yl)-N-ne opentyl-7H-pyrrolo [2,3-d]pyrimidin-2-amine [595];
(S)-N-(sec-butyl)-5-(1-isopropyl-2-methyl-1H-imidazo [4,5-b]pyridin-6-yl)-7H-pyrrolo [2,3-d]pyrimidin-2-amine [596];
N-(2-fluoro-2-methylpropyl)-5-(1-isopropyl-2-methyl-1H-imidazo [4,5-b]pyridin-6-yl)-7H-pyrrolo [2,3-d]pyrimidin-2-amine [597];
N-(2,2-difluoropropyl)-5-(1-isopropyl-2-methyl-1H-imidazo [4,5-b]pyridin-6-yl)-7H-pyrrolo [2,3-d]pyrimidin-2-amine [598];
5-(1-i sopropyl-2-methyl-1H-imidazo [4,5-b]pyridin-6-yl)-N-(2,2,2-trifluoroethyl)-7H-pyrrolo [2,3-d]pyrimidin-2-amine [599];
(R)-5-(1-i sop ropyl-2-methyl-1H-imidazo [4,5-b]pyridin-6-yl)-N-(1,1,1-trifluoropropan-2-yl)-7H-pyrrolo [2,3-d]pyrimidin-2-amine [600];
(S)-5-(1-i sop ropyl-2-methyl-1H-imidazo [4,5-b]pyridin-6-yl)-N-(1, 1, 1-trifluoropropan-2-yl)-7H-pyrrolo [2,3-d]pyrimidin-2-amine [601];
5-(1-isopropyl-2-methyl-1H-imidazo [4,5-b]pyridin-6-yl)-N-(3,3,3-trifluoropropyl)-7H-pyrrolo [2,3-d]pyrimidin-2-amine [602];
(S)-5-(1-i sop ropyl-2-methyl-1H-imidazo [4,5-b]pyridin-6-yl)-N-(1-methoxypropan-2-yl)-7H-pyrrolo [2,3-d]pyrimidin-2-amine [603];
6-(2-ethoxy-7H-pyrrolo [2,3-d]pyrimidin-5-yl)-1-isopropyl-2-methyl-1H-imidazo [4,5-b]pyridine [604];
N-((1-fluorocyclopropyl)methyl)-5-(1-i sopropyl-2-methyl-1H-imidazo [4,5-b]pyridin-6-yl)-7H-pyrrolo [2,3-d]pyrimidin-2-amine [605];
(R)-N-(1-cyclopropylethyl)-5-(1-i sop ropyl-2-methyl-1H-imidazo [4,5-b]pyridin-6-yl)-7H-pyrrolo [2,3-d]pyrimidin-2-amine [606];
5-(1-isopropyl-2-methyl-1H-imidazo [4,5-b]pyridin-6-yl)-N-((1-methylcyclopropyl)methyl)-7H-pyrrolo [2,3-d]pyrimidin-2-amine [607];
5-(1-isopropyl-2-methyl-1H-imidazo [4,5-b]pyridin-6-yl)-N-4 1-(trifluoromethyl)cyclopropyl)methyl)-7H-pyrrolo [2,3-d]pyrimidin-2-amine [608];
N-(2-cyclopropyl-2,2-difluoroethyl)-5-(1-i sop ropyl-2-methyl-1H-imidazo [4,5-b]pyridin-6-yl)-7H-pyrrolo [2,3-d]pyrimidin-2-amine [609];
N-(3,3-difluorocyclobutyl)-5-(1-i sopropyl-2-methyl-1H-imidazo [4,5-b]pyridin-6-yl)-7H-pyrrolo [2,3-d]pyrimidin-2-amine [610];
N-((1-fluorocyclobutyl)methyl)-5-(1-i sopropyl-2-methyl-1H-imidazo [4,5-b]pyridin-6-yl)-7H-pyrrolo [2,3-d]pyrimidin-2-amine [611];
N-(4,4-difluorocyclohexyl)-5-(1-i sopropyl-2-methyl-1H-imidazo [4,5-b] pyridin-6-yl)-7H-pyrrolo [2,3-d]pyrimidin-2-amine [612];
5-(1-isopropyl-2-methyl-1H-imidazo [4,5-b]pyridin-6-yl)-N-(cis-4-methoxycyclohexyl)-7H-pyrrolo [2,3-d]pyrimidin-2-amine [613];
N-((1-fluorocyclohexyl)methyl)-5-(1-i sopropyl-2-methyl-1H-imidazo [4,5-b]pyridin-6-yl)-7H-pyrrolo [2,3-d]pyrimidin-2-amine [614];
N-((4,4-difluorocyclohexyl)methyl)-5-(1-i sop ropyl-2-methyl-1H-imidazo [4,5-b]pyridin-6-yl)-7H-pyrrolo [2,3-d]pyrimidin-2-amine [615];
5-(1-isopropyl-2-methyl-1H-imidazo [4,5-b]pyridin-6-yl)-N-(1-methylpiperidin-4-yl)-7H-pyrrolo [2,3-d]pyrimidin-2-amine [616];
5-(1-i sopropyl-2-methyl-1H-imidazo [4,5-b]pyridin-6-yl)-N-((1-methylpiperidin-4-yl)methyl)-7H-pyrrolo [2,3-d]pyrimidin-2-amine [617];
5-(1-i sopropyl-2-methyl-1H-imidazo [4,5-b]pyridin-6-yl)-N-(tetrahydro-2H-pyran-4-yl)-7H-pyrrolo [2,3-d]pyrimidin-2-amine [618];
5-(1-i sopropyl-2-methyl-1H-imidazo [4,5-b]pyridin-6-yl)-N-(3-(4-methylpiperazin-1-yl)phenyl)-7H-pyrrolo [2,3-d]pyrimidin-2-amine [619];
5-(1-i sopropyl-2-methyl-1H-imidazo [4,5-b]pyridin-6-yl)-N-(pyridin-4-yl)-7H-pyrrolo [2,3-d]pyrimidin-2-amine [620];
5-(1-i sopropyl-2-methyl-1H-imidazo [4,5-b]pyridin-6-yl)-N-(2-(4-methylpiperazin-1-yl)pyridin-4-yl)-7H-pyrrolo [2,3-d] pyrimidin-2-amine [621];
5-(1-i sopropyl-2-methyl-1H-imidazo [4,5-b]pyridin-6-yl)-N-(6-(4-methylpiperazin-1-yl)pyridin-3-yl)-7H-pyrrolo [2,3-d] pyrimidin-2-amine [622];
5-(1-i sopropyl-2-methyl-1H-imidazo [4,5-b]pyridin-6-yl)-N-46-(4-methylpipe razin-1-yl)pyridin-3-yl)methyl)-7H-pyrrolo [2,3-d]pyrimidin-2-amine [623];
6-(5-(1-i sopropyl-2-methyl-1H-imidazo [4,5-b] pyridin-6-yl)-7H-pyrrolo [2,3-d]pyrimidin-2-yl)quinoline [624];
1-i sopropyl-2-methyl-6-(4-(1-methyl-1H-pyrazol-4-yl)-7H-pyrrolo [2,3-d]pyrimidin-5-yl)-1H-imidazo [4,5-b]pyridine [625];
5-(1-i sopropyl-2-methyl-1H-imidazo [4,5-b]pyridin-6-yl)-N-(trans-4-morpholinocyclohexyl)-7H-pyrrolo [2,3-d]pyrimidin-4-amine [626];
6-(2-cyclobutyl-7H-pyrrolo [2,3-d]pyrimidin-5-yl)-1-i sopropyl-2-methyl-1H-imidazo [4,5-c] pyridine [628];
5-(2,3-dimethyl-3H-imidazo [4,5-b]pyridin-5-yl)-N-(2-fluoro-2-methylpropyl)-7H-pyrrolo [2,3-d]pyrimidin-2-amine [629];
3-(2,2-difluoroethyl)-5-(2-isobutyl-7H-pyrrolo [2,3-d]pyrimidin-5-yl)-2-methyl-3H-imidazo [4,5-b]pyridine [631];
3-(2,2-difluoroethyl)-2-methyl-5-(2-(3,3,3-trifluoropropyl)-7H-pyrrolo [2,3-d]pyrimidin-5-yl)-3H-imidazo [4,5-b]pyridine [632];
5-(2-cyclopropyl-7H-pyrrolo [2,3-d]pyrimidin-5-yl)-3-(2,2-difluoroethyl)-2-methyl-3H-imidazo [4,5-b]pyridine [633];
5-(2-cyclobutyl-7H-pyrrolo [2,3-d]pyrimidin-5-yl)-3-(2,2-difluoroethyl)-2-methyl-3H-imidazo [4,5-b]pyridine [634];
5-(2-(cyclobutylmethyl)-7H-pyrrolo [2,3-d]pyrimidin-5-yl)-3-(2,2-difluoroethyl)-2-methyl-3H-imidazo [4,5-b]pyridine [635];
5-(3-(2,2-difluoroethyl)-2-methyl-3H-imidazo [4,5-b] pyridin-5-yl)-7H-pyrrolo [2,3-d]pyrimidin-2-amine [636];
5-(3-(2,2-difluoroethyl)-2-methyl-3H-imidazo [4,5-b] pyridin-5-yl)-N-methyl-7H-pyrrolo [2,3-d]pyrimidin-2-amine [637];
5-(3-(2,2-difluoroethyl)-2-methyl-3H-imidazo [4,5-b] pyridin-5-yl)-N-ethyl-7H-pyrrolo [2,3-d]pyrimidin-2-amine [638];
5-(3-(2,2-difluoroethyl)-2-methyl-3H-imidazo [4,5-b] pyridin-5-yl)-N-isopropyl-7H-pyrrolo [2,3-d]pyrimidin-2-amine [639];
5-(3-(2,2-difluoroethyl)-2-methyl-3H-imidazo [4,5-b] pyridin-5-yl)-N-isobutyl-7H-pyrrolo [2,3-d]pyrimidin-2-amine [640];
5-(3-(2,2-difluoroethyl)-2-methyl-3H-imidazo [4,5-b] pyridin-5-yl)-N-neopentyl-7H-pyrrolo [2,3-d]pyrimidin-2-amine [641];
(S)-N-(sec-butyl)-5-(3-(2,2-difluoroethyl)-2-methyl-3H-imidazo [4,5-b] pyridin-5-yl)-7H-pyrrolo [2,3-d]pyrimidin-2-amine [642];
5-(3-(2,2-difluoroethyl)-2-methyl-3H-imidazo [4,5-b] pyridin-5-yl)-N-(2-fluoro-2-methylpropyl)-7H-pyrrolo [2,3-d]pyrimidin-2-amine [643];
5-(3-(2,2-difluoroethyl)-2-methyl-3H-imidazo [4,5-b] pyridin-5-yl)-N-(2,2-difluoropropyl)-7H-pyrrolo [2,3-d]pyrimidin-2-amine [644];
5-(3-(2,2-difluoroethyl)-2-methyl-3H-imidazo [4,5-b] pyridin-5-yl)-N-(2,2,2-trifluoroethyl)-7H-pyrrolo [2,3-d]pyrimidin-2-amine [645];
(R)-5-(3-(2,2-difluoroethyl)-2-methyl-3H-imidazo [4,5-b]pyridin-5-yl)-N-(1, 1, 1-trifluoropropan-2-yl)-7H-pyrrolo [2,3-d] pyrimidin-2-amine [646];
(S)-5-(3-(2,2-difluoroethyl)-2-methyl-3H-imidazo [4,5-b]pyridin-5-yl)-N-(1, 1, 1-trifluoropropan-2-yl)-7H-pyrrolo [2,3-d] pyrimidin-2-amine [647];
5-(3-(2,2-difluoroethyl)-2-methyl-3H-imidazo [4,5-b] pyridin-5-yl)-N-(3 ,3 ,3-trifluoropropyl)-7H-pyrrolo [2,3-d]pyrimidin-2-amine [648];
(S)-5-(3-(2,2-difluoroethyl)-2-methyl-3H-imidazo [4,5-b]pyridin-5-yl)-N-(1-methoxypropan-2-yl)-7H-pyrrolo [2,3-d] pyrimidin-2-amine [649];
3-(2,2-difluoroethyl)-5-(2-ethoxy-7H-pyrrolo [2,3-d]pyrimidin-5-yl)-2-methyl-3H-imidazo [4,5-b]pyridine [650];
5-(3-(2,2-difluoroethyl)-2-methyl-3H-imidazo [4,5-b] pyridin-5-yl)-N-((1-fluorocyclopropyl)methyl)-7H-pyrrolo [2,3-d]pyrimidin-2-amine [651];
(R)-N-(1-cyclopropylethyl)-5-(3-(2,2-difluoroethyl)-2-methyl-3H-imidazo [4,5-b]pyridin-5-yl)-7H-pyrrolo [2,3-d]pyrimidin-2-amine [652];
5-(3-(2,2-difluoroethyl)-2-methyl-3H-imidazo [4,5-b] pyridin-5-yl)-N-((1-methylcyclopropyl)methyl)-7H-pyrrolo [2,3-d] pyrimidin-2-amine [653];
5-(3-(2,2-difluoroethyl)-2-methyl-3H-imidazo [4,5-b] pyridin-5-yl)-N-4 1-(trifluoromethyl)cyclopropyl)methyl)-7H-pyrrolo [2,3-d]pyrimidin-2-amine [654];
N-(2-cyclopropyl-2,2-difluoroethyl)-5-(3-(2,2-difluoroethyl)-2-methyl-3H-imidazo [4,5-b]pyridin-5-yl)-7H-pyrrolo [2,3-d] pyrimidin-2-amine [655];
N-(3,3-difluorocyclobutyl)-5-(3-(2,2-difluoroethyl)-2-methyl-3H-imidazo [4,5-b]pyridin-5-yl)-7H-pyrrolo [2,3-d]pyrimidin-2-amine [656];
5-(3-(2,2-difluoroethyl)-2-methyl-3H-imidazo [4,5-b] pyridin-5-yl)-N-((1-fluorocyclobutyl)methyl)-7H-pyrrolo [2,3-d]pyrimidin-2-amine [657];
N-(4,4-difluorocyclohexyl)-5-(3-(2,2-difluoroethyl)-2-methyl-3H-imidazo [4,5-b]pyridin-5-yl)-7H-pyrrolo [2,3-d]pyrimidin-2-amine [658];
5-(3-(2,2-difluoroethyl)-2-methyl-3H-imidazo [4,5-b] pyridin-5-yl)-N-(cis-4-methoxycyclohexyl)-7H-pyrrolo [2,3-d]pyrimidin-2-amine [659];
5-(3-(2,2-difluoroethyl)-2-methyl-3H-imidazo [4,5-b] pyridin-5-yl)-N-((1-fluorocyclohexyl)methyl)-7H-pyrrolo [2,3-d]pyrimidin-2-amine [660];
N-((4,4-difluorocyclohexyl)methyl)-5-(3-(2,2-difluoroethyl)-2-methyl-3H-imidazo [4,5-b] pyridin-5-yl)-7H-pyrrolo [2,3-d] pyrimidin-2-amine [661];
5-(3-(2,2-difluoroethyl)-2-methyl-3H-imidazo [4,5-b] pyridin-5-yl)-N-(1-methylpipe ridin-4-yl)-7H-pyrrolo [2,3-d]pyrimidin-2-amine [662];
5-(3-(2,2-difluoroethyl)-2-methyl-3H-imidazo [4,5-b] pyridin-5-yl)-N-((1-methylpiperidin-4-yl)methyl)-7H-pyrrolo [2,3-d]pyrimidin-2-amine [663];
5-(3-(2,2-difluoroethyl)-2-methyl-3H-imidazo [4,5-b] pyridin-5-yl)-N-(tetrahydro-2H-pyran-4-yl)-7H-pyrrolo [2,3-d]pyrimidin-2-amine [664];
5-(3-(2,2-difluoroethyl)-2-methyl-3H-imidazo [4,5-b] pyridin-5-yl)-N-(3-(4-methylpiperazin-1-yl)phenyl)-7H-pyrrolo [2,3-d]pyrimidin-2-amine [665];
5-(3-(2,2-difluoroethyl)-2-methyl-3H-imidazo [4,5-b] pyridin-5-yl)-N-(pyridin-4-yl)-7H-pyrrolo [2,3-d]pyrimidin-2-amine [666];
5-(3-(2,2-difluoroethyl)-2-methyl-3H-imidazo [4,5-b] pyridin-5-yl)-N-(2-(4-methylpiperazin-1-yl)pyridin-4-yl)-7H-pyrrolo [2,3-d]pyrimidin-2-amine [667];
5-(3-(2,2-difluoroethyl)-2-methyl-3H-imidazo [4,5-b] pyridin-5-yl)-N-(6-(4-methylpiperazin-1-yl)pyridin-3-yl)-7H-pyrrolo [2,3-d]pyrimidin-2-amine [668];
5-(3-(2,2-difluoroethyl)-2-methyl-3H-imidazo [4,5-b] pyridin-5-yl)-N-((6-(4-methylpipe razin-1-yl)pyridin-3-yl)methyl)-7H-pyrrolo [2,3-d] pyrimidin-2-amine [669];
6-(5-(3-(2,2-difluoroethyl)-2-methyl-3H-imidazo [4,5-1)] pyridin-5-yl)-7H-pyrrolo [2,3-d]pyrimidin-2-yl)quinoline [670];
3-(2,2-difluoroethyl)-2-methyl-5-(4-(1-methyl-1H-pyrazol-4-yl)-7H-pyrrolo [2,3-d]pyrimidin-5-yl)-3H-imidazo [4,5-b]pyridine [671];
5-(3-(2,2-difluoroethyl)-2-methyl-3H-imidazo [4,5-b] pyridin-5-yl)-N-(trans-4-morpholinocyclohexyl)-7H-pyrrolo [2,3-d] pyrimidin-4-amine [672];
5-(2-isobutyl-7H-pyrrolo [2,3-d] pyrimidin-5-yl)-3-isopropyl-2-methyl-3H-imidazo [4,5-b] pyridine [674];
3-isopropyl-2-methyl-5-(2-(3,3,3-trifluoropropyl)-7H-pyrrolo [2,3-d]pyrimidin-5-yl)-3H-imidazo [4,5-b] pyridine [675];
5-(2-cyclopropyl-7H-pyrrolo [2,3-d]pyrimidin-5-yl)-3-isopropyl-2-methyl-3H-imidazo [4,5]-14yridine [676];
5-(2-cyclobutyl-7H-pyrrolo [2,3-d]pyrimidin-5-yl)-3-isopropyl-2-methyl-3H-imidazo [4,5]-14yridine [677];
5-(2-(cyclobutylmethyl)-7H-pyrrolo [2,3-d]pyrimidin-5-yl)-3-isopropyl-2-methyl-3H-imidazo [4,5-b] pyridine [678];
5-(3-isopropyl-2-methyl-3H-imidazo [4,5-b]pyridin-5-yl)-7H-pyrrolo [2,3-d] pyrimidin-2-amine [679];
5-(3-isopropyl-2-methyl-3H-imidazo [4,5-b]pyridin-5-yl)-N-methyl-7H-pyrrolo [2,3-d]pyrimidin-2-amine [680];
N-ethyl-5-(3-isop ropyl-2-methyl-3H-imidazo [4,5-b]pyridin-5-yl)-7H-pyrrolo [2,3-d] pyrimidin-2-amine [681];
N-i sopropyl-5-(3-isopropyl-2-methyl-3H-imidazo [4,5-b]pyridin-5-yl)-7H-pyrrolo [2,3-d]pyrimidin-2-amine [682];
N-i sobutyl-5-(3-isop ropyl-2-methyl-3H-imidazo [4,5-b]pyridin-5-yl)-7H-pyrrolo [2,3-d] pyrimidin-2-amine [683];
5-(3-isopropyl-2-methyl-3H-imidazo [4,5-b]pyridin-5-yl)-N-neopentyl-7H-pyrrolo [2,3-d]pyrimidin-2-amine [684];
(S)-N-(sec-butyl)-5-(3-i sopropyl-2-methyl-3H-imidazo [4,5-b] pyridin-5-yl)-7H-pyrrolo [2,3-d]pyrimidin-2-amine [685];
N-(2-fluoro-2-methylpropyl)-5-(3-isopropyl-2-methyl-3H-imidazo [4,5-b]pyridin-5-yl)-7H-pyrrolo [2,3-d]pyrimidin-2-amine [686];
N-(2,2-difluoropropyl)-5-(3-isopropyl-2-methyl-3H-imidazo [4,5-b]pyridin-5-yl)-7H-pyrrolo [2,3-d]pyrimidin-2-amine [687];
5-(3-isopropyl-2-methyl-3H-imidazo [4,5-b]pyridin-5-yl)-N-(2,2,2-trifluoroethyl)-7H-pyrrolo [2,3-d]pyrimidin-2-amine [688];
(R)-5-(3-i sop ropyl-2-methyl-3H-imidazo [4,5-b]pyridin-5-yl)-N-(1, 1, 1-trifluoropropan-2-yl)-7H-pyrrolo [2,3-d]pyrimidin-2-amine [689];
(S)-5-(3-i sop ropyl-2-methyl-3H-imidazo [4,5-b]pyridin-5-yl)-N-(1, 1, 1-trifluoropropan-2-yl)-7H-pyrrolo [2,3-d]pyrimidin-2-amine [690];
5-(3-isopropyl-2-methyl-3H-imidazo [4,5-b]pyridin-5-yl)-N-(3 ,3 ,3-trifluoropropyl)-7H-pyrrolo [2,3-d]pyrimidin-2-amine [691];
(S)-5-(3-i sop ropyl-2-methyl-3H-imidazo [4,5-b]pyridin-5-yl)-N-(1-methoxypropan-2-yl)-7H-pyrrolo [2,3-d]pyrimidin-2-amine [692];
5-(2-ethoxy-7H-pyrrolo [2,3-d] pyrimidin-5-yl)-3-isopropyl-2-methyl-3H-imidazo [4,5-b]pyridine [693];
N-((1-fluorocyclopropyl)methyl)-5-(3-isopropyl-2-methyl-3H-imidazo [4,5-b]pyridin-5-yl)-7H-pyrrolo [2,3-d]pyrimidin-2-amine [694];
(R)-N-(1-cyclopropylethyl)-5-(3-i sop ropyl-2-methyl-3H-imidazo [4,5-b]pyridin-5-yl)-7H-pyrrolo [2,3-d]pyrimidin-2-amine [695];
5-(3-isopropyl-2-methyl-3H-imidazo [4,5-b]pyridin-5-yl)-N-((1-methylcyclopropyl)methyl)-7H-pyrrolo [2,3-d]pyrimidin-2-amine [696];
5-(3-isopropyl-2-methyl-3H-imidazo [4,5-b]pyridin-5-yl)-N-4 1-(trifluoromethyl)cyclopropyl)methyl)-7H-pyrrolo [2,3-d]pyrimidin-2-amine [697];
N-(2-cyclopropyl-2,2-difluoroethyl)-5-(3-i sop ropyl-2-methyl-3H-imidazo [4,5-b]pyridin-5-yl)-7H-pyrrolo [2,3-d]pyrimidin-2-amine [698];
N-(3,3-difluorocyclobutyl)-5-(3-isopropyl-2-methyl-3H-imidazo [4,5-b]pyridin-5-yl)-7H-pyrrolo [2,3-d]pyrimidin-2-amine [699];
N-((1-fluorocyclobutypmethyl)-5-(3-isopropyl-2-methyl-3H-imidazo [4,5-b]pyridin-5-yl)-7H-pyrrolo [2,3-d]pyrimidin-2-amine [700];
N-(4,4-difluorocyclohexyl)-5-(3-isopropyl-2-methyl-3H-imidazo [4,5-1)] pyridin-5-yl)-7H-pyrrolo [2,3-d]pyrimidin-2-amine [701];
5-(3-isopropyl-2-methyl-3H-imidazo [4,5-b]pyridin-5-yl)-N-(cis-4-methoxycyclohexyl)-7H-pyrrolo [2,3-d]pyrimidin-2-amine [702];
N-((1-fluorocyclohexyl)methyl)-5-(3-isopropyl-2-methyl-3H-imidazo [4,5-b]pyridin-5-yl)-7H-pyrrolo [2,3-d]pyrimidin-2-amine [703];
N-((4,4-difluorocyclohexyl)methyl)-5-(3-i sop ropyl-2-methyl-3H-imidazo [4,5-b]pyridin-5-yl)-7H-pyrrolo [2,3-d]pyrimidin-2-amine [704];
5-(3 sopropyl-2-methyl-3H-imidazo [4,5-b]pyridin-5-yl)-N-(1-methylpiperidin-4-yl)-7H-pyrrolo [2,3-d]pyrimidin-2-amine [705];
5-(3 sopropyl-2-methyl-3H-imidazo [4,5-b]pyridin-5-yl)-N-((1-methylpiperidin-4-yl)methyl)-7H-pyrrolo [2,3-d]pyrimidin-2-amine [706];
5-(3 sopropyl-2-methyl-3H-imidazo [4,5-b]pyridin-5-yl)-N-(tetrahydro-2H-pyran-4-yl)-7H-pyrrolo [2,3-d]pyrimidin-2-amine [707];
5-(3 sopropyl-2-methyl-3H-imidazo [4,5-b]pyridin-5-yl)-N-(3-(4-methylpiperazin-1-yl)phenyl)-7H-pyrrolo [2,3-d]pyrimidin-2-amine [708];
5-(3 sopropyl-2-methyl-3H-imidazo [4,5-b]pyridin-5-yl)-N-(pyridin-4-yl)-7H-pyrrolo [2,3-d]pyrimidin-2-amine [709];
5-(3 sopropyl-2-methyl-3H-imidazo [4,5-b]pyridin-5-yl)-N-(2-(4-methylpiperazin-1-yl)pyridin-4-yl)-7H-pyrrolo [2,3-d] pyrimidin-2-amine [710];
5-(3 sopropyl-2-methyl-3H-imidazo [4,5-b]pyridin-5-yl)-N-(6-(4-methylpiperazin-1-yl)pyridin-3-yl)-7H-pyrrolo [2,3-d] pyrimidin-2-amine [711];
5-(3 sopropyl-2-methyl-3H-imidazo [4,5-b]pyridin-5-yl)-N-46-(4-methylpipe razin-1-yl)pyridin-3-yl)methyl)-7H-pyrrolo [2,3-d]pyrimidin-2-amine [712];
6-(5-(3-i sopropyl-2-methyl-3H-imidazo [4,5-b]pyridin-5-yl)-7H-pyrrolo [2,3-d]pyrimidin-2-yl)quinoline [713];
3-i sopropyl-2-methyl-5-(4-(1-methyl-1H-pyrazol-4-yl)-7H-pyrrolo [2,3-d]pyrimidin-5-yl)-3H-imidazo[4,5-b]pyridine [714];
5-(3 sopropyl-2-methyl-3H-imidazo [4,5-b]pyridin-5-yl)-N-(trans-4-morpholinocyclohexyl)-7H-pyrrolo [2,3-d]pyrimidin-4-amine [715];
6-(2-isobutyl-7H-pyrrolo [2,3-d] pyrimidin-5-yl)-[1,2,4]triazolo [1,5-a] pyridine [717];
5-([1,2,4]triazolo [1,5-a]pyridin-6-yl)-N-(tert-butyl)-7H-pyrrolo [2,3-d]pyrimidin-2-amine [718];
6-(2-(3,3,3-trifluoropropyl)-7H-pyrrolo [2,3-d]pyrimidin-5-yl)-[1,2,4]triazolo [1,5-a]pyridine [719];
6-(2-cyclopropyl-7H-pyrrolo [2,3-d]pyrimidin-5-yl)-[1,2,4]triazolo [1,5-a]pyridine [720];
6-(2-cyclobutyl-7H-pyrrolo [2,3-d]pyrimidin-5-yl)-[1,2,4]triazolo [1,5-a]pyridine [721];
6-(2-(cyclobutylmethyl)-7H-pyrrolo [2,3-d]pyrimidin-5-yl)-[1,2,4]triazolo [1,5-a]pyridine [722];
5-([1,2,4]triazolo [1,5-a]pyridin-6-yl)-7H-pyrrolo [2,3-d]pyrimidin-2-amine [723];
5-([1,2,4]triazolo [1,5-a]pyridin-6-yl)-N-methyl-7H-pyrrolo [2,3-d] pyrimidin-2-amine [724];
5-([1,2,4]triazolo [1,5-a]pyridin-6-yl)-N-ethyl-7H-pyrrolo [2,3-d]pyrimidin-2-amine [725];
5-([1,2,4]triazolo [1,5-a]pyridin-6-yl)-N-isopropyl-7H-pyrrolo [2,3-d]pyrimidin-2-amine [726];
5-([1,2,4]triazolo [1,5-a]pyridin-6-yl)-N-isobutyl-7H-pyrrolo [2,3-d]pyrimidin-2-amine [727];
5-([1,2,4]triazolo [1,5-a]pyridin-6-yl)-N-neopentyl-7H-pyrrolo [2,3-d]pyrimidin-2-amine [728];
(S)-5-([1,2,4]triazolo [1,5-a] pyridin-6-yl)-N-(sec-butyl)-7H-pyrrolo [2,3-d]pyrimidin-2-amine [729];
5-([1,2,4]triazolo [1,5-a]pyridin-6-yl)-N-(2,2-difluoroethyl)-7H-pyrrolo [2,3-d] pyrimidin-2-amine [730];
5-([1,2,4]triazolo [1,5-a]pyridin-6-yl)-N-(2-fluoro-2-methylpropyl)-7H-pyrrolo [2,3-d]pyrimidin-2-amine [731];
5-([1,2,4]triazolo [1,5-a]pyridin-6-yl)-N-(2,2-difluoropropyl)-7H-pyrrolo [2,3-d]pyrimidin-2-amine [732];
5-([1,2,4]triazolo [1,5-a]pyridin-6-yl)-N-(2,2,2-trifluoroethyl)-7H-pyrrolo [2,3-d]pyrimidin-2-amine [733];
(R)-5-([1,2,4]triazolo [1,5-a] pyridin-6-yl)-N-(1,1,1-trifluoropropan-2-yl)-7H-pyrrolo [2,3-d]pyrimidin-2-amine [734];
(S)-5-([1,2,4]triazolo [1,5-a] pyridin-6-yl)-N-(1,1,1-trifluoropropan-2-yl)-7H-pyrrolo [2,3-d]pyrimidin-2-amine [735];
5-([1,2,4]triazolo [1,5-a]pyridin-6-yl)-N-(3,3,3-trifluoropropyl)-7H-pyrrolo [2,3-d]pyrimidin-2-amine [736];
(S)-5-([1,2,4]triazolo [1,5-a] pyridin-6-yl)-N-(1-methoxypropan-2-yl)-7H-pyrrolo [2,3-d]pyrimidin-2-amine [737];
6-(2-ethoxy-7H-pyrrolo [2,3-d] pyrimidin-5-yl)-[1,2,4]triazolo [1,5-a]pyridine [738];
5-([1,2,4]triazolo [1,5-a]pyridin-6-yl)-N-((1-fluorocyclopropyl)methyl)-7H-pyrrolo [2,3-d]pyrimidin-2-amine [739];
(R)-5-([1,2,4]triazolo [1,5-a] pyridin-6-yl)-N-(1-cyclopropylethyl)-7H-pyrrolo [2,3-d] pyrimidin-2-amine [740];
5-([1,2,4]triazolo [1,5-a]pyridin-6-yl)-N-((1-methylcyclopropyl)methyl)-7H-pyrrolo [2,3-d]pyrimidin-2-amine [741];
5-([1,2,4]triazolo [1,5-a]pyridin-6-yl)-N-41-(trifluoromethyl)cyclopropyl)methyl)-7H-pyrrolo [2,3-d]pyrimidin-2-amine [742];
5-([1,2,4]triazolo [1,5-a]pyridin-6-yl)-N-(2-cyclopropyl-2,2-difluoroethyl)-7H-pyrrolo [2,3-d]pyrimidin-2-amine [743];
5-([1,2,4]triazolo [1,5-a]pyridin-6-yl)-N-(3,3-difluorocyclobutyl)-7H-pyrrolo [2,3-d]pyrimidin-2-amine [744];
5-([1,2,4]triazolo [1,5-a]pyridin-6-yl)-N-((1-fluorocyclobutyl)methyl)-7H-pyrrolo [2,3-d]pyrimidin-2-amine [745];
5-([1,2,4]triazolo [1,5-a]pyridin-6-yl)-N-(4,4-difluorocyclohexyl)-7H-pyrrolo [2,3-d]pyrimidin-2-amine [746];
5-([1,2,4]triazolo [1,5-a]pyridin-6-yl)-N-(cis-4-methoxycyclohexyl)-7H-pyrrolo [2,3-d]pyrimidin-2-amine [747];
5-([1,2,4]triazolo [1,5-a]pyridin-6-yl)-N-((1-fluorocyclohexyl)methyl)-7H-pyrrolo [2,3-d]pyrimidin-2-amine [748];
5-([1,2,4]triazolo [1,5-a]pyridin-6-yl)-N-((4,4-difluorocyclohexyl)methyl)-7H-pyrrolo [2,3-d]pyrimidin-2-amine [749];
5-([1,2,4]triazolo [1,5-a]pyridin-6-yl)-N-(1-methylpiperidin-4-yl)-7H-pyrrolo [2,3-d] pyrimidin-2-amine [750];
5-([1,2,4]triazolo [1,5-a]pyridin-6-yl)-N-((1-methylpiperidin-4-yl)methyl)-7H-pyrrolo [2,3-d]pyrimidin-2-amine [751];
5-([1,2,4]triazolo [1,5-a]pyridin-6-yl)-N-(tetrahydro-2H-pyran-4-yl)-7H-pyrrolo [2,3-d]pyrimidin-2-amine [752];
5-([1,2,4]triazolo [1,5-a]pyridin-6-yl)-N-(3-(4-methylpipe razin-1-yl)phenyl)-7H-pyrrolo [2,3-d]pyrimidin-2-amine [753];
5-([1,2,4]triazolo [1,5-a]pyridin-6-yl)-N-(pyridin-4-yl)-7H-pyrrolo [2,3-d]pyrimidin-2-amine [754];
5-([1,2,4]triazolo [1,5-a]pyridin-6-yl)-N-(2-(4-methylpipe razin-1-yl)pyridin-4-yl)-7H-pyrrolo [2,3-d]pyrimidin-2-amine [755];
5-([1,2,4]triazolo [1,5-a]pyridin-6-yl)-N-(6-(4-methylpipe razin-1-yl)pyridin-3-yl)-7H-pyrrolo [2,3-d]pyrimidin-2-amine [756];
5-([1,2,4]triazolo [1,5-a]pyridin-6-yl)-N-46-(4-methylpiperazin-1-yl)pyridin-3-yl)methyl)-7H-pyrrolo [2,3-d]pyrimidin-2-amine [757];
6-(5-([1,2,4]triazolo [1,5-a] pyridin-6-yl)-7H-pyrrolo [2,3-d]pyrimidin-2-yl)quinoline [758];
6-(4-(1-methyl-1H-pyrazol-4-yl)-7H-pyrrolo [2,3-d] pyrimidin-5-yl)-[1,2,4]triazolo [1,5-a] pyridine [759];
5-([1,2,4]triazolo [1,5-a]pyridin-6-yl)-N-(trans-4-morpholinocyclohexyl)-7H-pyrrolo [2,3-d]pyrimidin-4-amine [760];
8-fluoro-6-(2-isobutyl-7H-pyrrolo [2,3-d]pyrimidin-5-yl)-[1,2,4]triazolo [1,5-a] pyridine [762];
8-fluoro -64243,3,3-trifluoropropyl)-7H-pyrrolo [2,3-d] pyrimidin-5-yl)-[1,2,4]triazolo [1,5-a]pyridine [763];
6-(2-cyclopropyl-7H-pyrrolo [2,3-d]pyrimidin-5-yl)-8-fluoro-[1,2,4]triazolo [1,5-a] pyridine [764];
6-(2-cyclobutyl-7H-pyrrolo [2,3-d]pyrimidin-5-yl)-8-fluoro-[1,2,4]triazolo [1,5-a]pyridine [765];
6-(2-(cyclobutylmethyl)-7H-pyrrolo [2,3-d]pyrimidin-5-yl)-8-fluoro-[1,2,4]triazolo [1,5-a]pyridine [766];
5-(8-fluoro-[1,2,4]triazolo [1,5-a]pyridin-6-yl)-7H-pyrrolo [2,3-d]pyrimidin-2-amine [767];
5-(8-fluoro-[1,2,4]triazolo [1,5-a]pyridin-6-yl)-N-methyl-7H-pyrrolo [2,3-d]pyrimidin-2-amine [768];
N-ethyl-5-(8-fluoro-[1,2,4]triazolo [1,5-a]pyridin-6-yl)-7H-pyrrolo [2,3-d] pyrimidin-2-amine [769];
5-(8-fluoro-[1,2,4]triazolo [1,5-a]pyridin-6-yl)-N-isopropyl-7H-pyrrolo [2,3-d]pyrimidin-2-amine [770];
5-(8-fluoro-[1,2,4]triazolo [1,5-a]pyridin-6-yl)-N-isobutyl-7H-pyrrolo [2,3-d] pyrimidin-2-amine [771];
5-(8-fluoro-[1,2,4]triazolo[1,5-a]pyridin-6-yl)-N-neopentyl-7H-pyrrolo [2,3-d] pyrimidin-2-amine [772];
(S)-N-(sec-butyl)-5-(8-fluoro-[1,2,4]triazolo [1,5-a]pyridin-6-yl)-7H-pyrrolo [2,3-d]pyrimidin-2-amine [773];
N-(2-fluoro-2-methylpropyl)-5-(8-fluoro-[1,2,4]triazolo [1,5-a]pyridin-6-yl)-7H-pyrrolo [2,3-d]pyrimidin-2-amine [774];
N-(2,2-difluoropropyl)-5-(8-fluoro-[1,2,4]triazolo [1,5-a]pyridin-6-yl)-7H-pyrrolo [2,3-d]pyrimidin-2-amine [775];
5-(8-fluoro-[1,2,4]triazolo [1,5-a]pyridin-6-yl)-N-(2,2,2-trifluoroethyl)-7H-pyrrolo [2,3-d]pyrimidin-2-amine [776];
(R)-5-(8-fluoro-[1,2,4]triazolo [1,5-a]pyridin-6-yl)-N-(1,1,1-trifluoropropan-2-yl)-7H-pyrrolo [2,3-d]pyrimidin-2-amine [777];
(S)-5-(8-fluoro-[1,2,4]triazolo [1,5-a]pyridin-6-yl)-N-(1,1,1-trifluoropropan-2-yl)-7H-pyrrolo [2,3-d]pyrimidin-2-amine [778];
5-(8-fluoro-[1,2,4]triazolo [1,5-a]pyridin-6-yl)-N-(3,3,3-trifluoropropyl)-7H-pyrrolo [2,3-d]pyrimidin-2-amine [779];
(S)-5-(8-fluoro-[1,2,4]triazolo [1,5-a]pyridin-6-yl)-N-(1-methoxypropan-2-yl)-7H-pyrrolo [2,3-d]pyrimidin-2-amine [780];
6-(2-ethoxy-7H-pyrrolo [2,3-d] pyrimidin-5-yl)-8-fluoro-[1,2,4]triazolo [1,5-a]pyridine [781];
5-(8-fluoro-[1,2,4]triazolo [1,5-a]pyridin-6-yl)-N-((1-fluorocyclopropyl)methyl)-7H-pyrrolo [2,3-d]pyrimidin-2-amine [782];
(R)-N-(1-cyclopropylethyl)-5-(8-fluoro-[1,2,4]triazolo [1,5-a]pyridin-6-yl)-7H-pyrrolo [2,3-d]pyrimidin-2-amine [783];
5-(8-fluoro-[1,2,4]triazolo [1,5-a]pyridin-6-yl)-N-((1-methylcyclopropyl)methyl)-7H-pyrrolo [2,3-d]pyrimidin-2-amine [784];
5-(8-fluoro-[1,2,4]triazolo [1,5-a]pyridin-6-yl)-N-41-(trifluoromethyl)cyclopropyl)methyl)-7H-pyrrolo [2,3-d]pyrimidin-2-amine [785];
N-(2-cyclopropyl-2,2-difluoroethyl)-5-(8-fluoro-[1,2,4]triazolo [1,5-a]pyridin-6-yl)-7H-pyrrolo [2,3-d]pyrimidin-2-amine [786];
N-(3,3-difluorocyclobutyl)-5-(8-fluoro-[1,2,4]triazolo [1,5-a]pyridin-6-yl)-7H-pyrrolo [2,3-d]pyrimidin-2-amine [787];
5-(8-fluoro-[1,2,4]triazolo [1,5-a]pyridin-6-yl)-N-((1-fluorocyclobutyl)methyl)-7H-pyrrolo [2,3-d]pyrimidin-2-amine [788];
N-(4,4-difluorocyclohexyl)-5-(8-fluoro-[1,2,4]triazolo [1,5-a]pyridin-6-yl)-7H-pyrrolo [2,3-d]pyrimidin-2-amine [789];
5-(8-fluoro-[1,2,4]triazolo [1,5-a]pyridin-6-yl)-N-(cis-4-methoxycyclohexyl)-7H-pyrrolo [2,3-d]pyrimidin-2-amine [790];
5-(8-fluoro-[1,2,4]triazolo [1,5-a]pyridin-6-yl)-N-((1-fluorocyclohexyl)methyl)-7H-pyrrolo [2,3-d]pyrimidin-2-amine [791];
N-((4,4-difluorocyclohexyl)methyl)-5-(8-fluoro-[1,2,4]triazolo [1,5-a]pyridin-6-yl)-7H-pyrrolo [2,3-d]pyrimidin-2-amine [792];
5-(8-fluoro-[1,2,4]triazolo [1,5-a]pyridin-6-yl)-N-(1-methylpipe ridin-4-yl)-7H-pyrrolo [2,3-d]pyrimidin-2-amine [793];
5-(8-fluoro-[1,2,4]triazolo [1,5-a]pyridin-6-yl)-N-((1-methylpiperidin-4-yl)methyl)-7H-pyrrolo [2,3-d]pyrimidin-2-amine [794];
5-(8-fluoro-[1,2,4]triazolo [1,5-a]pyridin-6-yl)-N-(tetrahydro-2H-pyran-4-yl)-7H-pyrrolo [2,3-d]pyrimidin-2-amine [795];
5-(8-fluoro-[1,2,4]triazolo [1,5-a]pyridin-6-yl)-N-(3-(4-methylpiperazin-1-yl)phenyl)-7H-pyrrolo [2,3-d]pyrimidin-2-amine [796];
5-(8-fluoro-[1,2,4]triazolo [1,5-a]pyridin-6-yl)-N-(pyridin-4-yl)-7H-pyrrolo [2,3-d] pyrimidin-2-amine [797];
5-(8-fluoro-[1,2,4]triazolo [1,5-a]pyridin-6-yl)-N-(2-(4-methylpiperazin-1-yl)pyridin-4-yl)-7H-pyrrolo [2,3-d]pyrimidin-2-amine [798];
5-(8-fluoro-[1,2,4]triazolo [1,5-a]pyridin-6-yl)-N-(6-(4-methylpiperazin-1-yl)pyridin-3-yl)-7H-pyrrolo [2,3-d]pyrimidin-2-amine [799];
5-(8-fluoro-[1,2,4]triazolo [1,5-a]pyridin-6-yl)-N-46-(4-methylpiperazin-1-yl)pyridin-3-yl)methyl)-7H-pyrrolo [2,3-d]pyrimidin-2-amine [800];
6-(5-(8-fluoro-[1,2,4]triazolo [1,5-a]pyridin-6-yl)-7H-pyrrolo [2,3-d] pyrimidin-2-yl)quinoline [801];
8-fluoro-6-(4-(1-methyl-1H-pyrazol-4-yl)-7H-pyrrolo [2,3-d]pyrimidin-5-yl)41,2,41triazolo [1,5-a] pyridine [802];
5-(8-fluoro-[1,2,4]triazolo [1,5-a]pyridin-6-yl)-N-(trans-4-morpholinocyclohexyl)-7H-pyrrolo [2,3-d]pyrimidin-4-amine [803];
5-([1,2,4]triazolo [1,5-a]pyridin-7-yl)-N-isopropyl-7H-pyrrolo [2,3-d]pyrimidin-2-amine [804];
5-([1,2,4]triazolo [1,5-a]pyridin-7-yl)-N-(2-fluoro-2-methylpropyl)-7H-pyrrolo [2,3-d]pyrimidin-2-amine [805];
(R)-5-([1,2,4]triazolo [1,5-a] pyridin-7-yl)-N-(1,1,1-trifluoropropan-2-yl)-7H-pyrrolo [2,3-d]pyrimidin-2-amine [806];
(S)-5-([1,2,4]triazolo [1,5-a] pyridin-7-yl)-N-(1,1,1-trifluoropropan-2-yl)-7H-pyrrolo [2,3-d]pyrimidin-2-amine [807];
5-([1,2,4]triazolo [1,5-a]pyridin-7-yl)-N-46-(4-methylpiperazin-1-yl)pyridin-3-yl)methyl)-7H-pyrrolo [2,3-d]pyrimidin-2-amine [808];
6-(2-isobutyl-7H-pyrrolo [2,3-d] pyrimidin-5-yl)-1-methyl-1H-benzo [d] [1,2,3]triazole [810];
1-methyl-6-(2-(3,3,3-trifluoropropyl)-7H-pyrrolo [2,3-d]pyrimidin-5-yl)-1H-benzo[d] [1,2,3]triazole [811];
6-(2-cyclopropyl-7H-pyrrolo [2,3-d]pyrimidin-5-yl)-1-methyl-1H-benzo [d] [1,2,3]triazole [812];
6-(2-cyclobutyl-7H-pyrrolo [2,3-d]pyrimidin-5-yl)-1-methyl-1H-benzo [d] [1,2,3]triazole [813];
6-(2-(cyclobutylmethyl)-7H-pyrrolo [2,3-d]pyrimidin-5-yl)-1-methyl-1H-benzo [d] [1,2,3]triazole [814];
5-(1-methyl-1H-benzo[ d] [1,2,3]triazol-6-yl)-7H-pyrrolo [2,3-d]pyrimidin-2-amine [815];
N-methyl-5-(1-methyl-1H-benzo [d] [1,2,3]triazol-6-yl)-7H-pyrrolo [2,3-d] pyrimidin-2-amine [816];
N-ethyl-5-(1-methyl-1H-benzo [d] [1,2,3]triazol-6-yl)-7H-pyrrolo [2,3-d]pyrimidin-2-amine [817];
N-isopropyl-5-(1-methyl-1H-benzo [d] [1,2,3]triazol-6-yl)-7H-pyrrolo [2,3-d] pyrimidin-2-amine [818];
N-isobutyl-5-(1-methyl-1H-benzo [d] [1,2,3]triazol-6-yl)-7H-pyrrolo [2,3-d]pyrimidin-2-amine [818];
5-(1-methyl-1H-benzo [d] [1,2,3]triazol-6-yl)-N-neopentyl-7H-pyrrolo [2,3-d]pyrimidin-2-amine [819];
(S)-N-(sec-butyl)-5-(1-methyl-1H-benzo[d] [1,2,3]triazol-6-yl)-7H-pyrrolo [2,3-d]pyrimidin-2-amine [821];
N-(2-fluoro-2-methylpropyl)-5-(1-methyl-1H-benzo[d][1,2,3]triazol-6-yl)-7H-pyrrolo[2,3-d]pyrimidin-2-amine [822];
N-(2,2-difluoropropyl)-5-(1-methyl-1H-benzo[d][1,2,3]triazol-6-yl)-7H-pyrrolo[2,3-d]pyrimidin-2-amine [823];
5-(1-methyl-1H-benzo[d][1,2,3]triazol-6-yl)-N-(2,2,2-trifluoroethyl)-7H-pyrrolo[2,3-d]pyrimidin-2-amine [824];
(R)-5-(1-methyl-1H-benzo[d][1,2,3]triazol-6-yl)-N-(1,1,1-trifluoropropan-2-yl)-7H-pyrrolo[2,3-d]pyrimidin-2-amine [825];
(S)-5-(1-methyl-1H-benzo[d][1,2,3]triazol-6-yl)-N-(1,1,1-trifluoropropan-2-yl)-7H-pyrrolo[2,3-d]pyrimidin-2-amine [826];
5-(1-methyl-1H-benzo[d][1,2,3]triazol-6-yl)-N-(3,3,3-trifluoropropyl)-7H-pyrrolo[2,3-d]pyrimidin-2-amine [827];
(S)-N-(1-methoxypropan-2-yl)-5-(1-methyl-1H-benzo[d][1,2,3]triazol-6-yl)-7H-pyrrolo[2,3-d]pyrimidin-2-amine [828];
6-(2-ethoxy-7H-pyrrolo[2,3-d]pyrimidin-5-yl)-1-methyl-1H-benzo[d][1,2,3]triazole [829];
N-((1-fluorocyclopropyl)methyl)-5-(1-methyl-1H-benzo[d][1,2,3]triazol-6-yl)-7H-pyrrolo[2,3-d]pyrimidin-2-amine [830];
(R)-N-(1-cyclopropylethyl)-5-(1-methyl-1H-benzo[d][1,2,3]triazol-6-yl)-7H-pyrrolo[2,3-d]pyrimidin-2-amine [831];
5-(1-methyl-1H-benzo[d][1,2,3]triazol-6-yl)-N-((1-methylcyclopropyl)methyl)-7H-pyrrolo[2,3-d]pyrimidin-2-amine [832];
5-(1-methyl-1H-benzo[d][1,2,3]triazol-6-yl)-N-41-(trifluoromethyl)cyclopropyl)methyl)-7H-pyrrolo[2,3-d]pyrimidin-2-amine [833];
N-(2-cyclopropyl-2,2-difluoroethyl)-5-(1-methyl-1H-benzo[d][1,2,3]triazol-6-yl)-7H-pyrrolo[2,3-d]pyrimidin-2-amine [834];
N-(3,3-difluorocyclobutyl)-5-(1-methyl-1H-benzo[d][1,2,3]triazol-6-yl)-7H-pyrrolo[2,3-d]pyrimidin-2-amine [835];
N-((1-fluorocyclobutyl)methyl)-5-(1-methyl-1H-benzo[d][1,2,3]triazol-6-yl)-7H-pyrrolo[2,3-d]pyrimidin-2-amine [836];
N-(4,4-difluorocyclohexyl)-5-(1-methyl-1H-benzo[d][1,2,3]triazol-6-yl)-7H-pyrrolo[2,3-d]pyrimidin-2-amine [837];
N-(cis-4-methoxycyclohexyl)-5-(1-methyl-1H-benzo[d][1,2,3]triazol-6-yl)-7H-pyrrolo[2,3-d]pyrimidin-2-amine [838];
N-((1-fluorocyclohexyl)methyl)-5-(1-methyl-1H-benzo[d][1,2,3]triazol-6-yl)-7H-pyrrolo[2,3-d]pyrimidin-2-amine [839];
N-((4,4-difluorocyclohexyl)methyl)-5-(1-methyl-1H-benzo[d][1,2,3]triazol-6-yl)-7H-pyrrolo[2,3-d]pyrimidin-2-amine [840];
5-(1-methyl-1H-benzo[d][1,2,3]triazol-6-yl)-N-(1-methylpiperidin-4-yl)-7H-pyrrolo[2,3-d]pyrimidin-2-amine [841];
5-(1-methyl-1H-benzo[d][1,2,3]triazol-6-yl)-N-((1-methylpiperidin-4-yl)methyl)-7H-pyrrolo[2,3-d]pyrimidin-2-amine [842];
5-(1-methyl-1H-benzo[d][1,2,3]triazol-6-yl)-N-(tetrahydro-2H-pyran-4-yl)-7H-pyrrolo[2,3-d]pyrimidin-2-amine [843];
5-(1-methyl-1H-benzo[d][1,2,3]triazol-6-yl)-N-(3-(4-methylpiperazin-1-yl)phenyl)-7H-pyrrolo[2,3-d]pyrimidin-2-amine [844];
5-(1-methyl-1H-benzo[d][1,2,3]triazol-6-yl)-N-(pyridin-4-yl)-7H-pyrrolo[2,3-d]pyrimidin-2-amine [845];
5-(1-methyl-1H-benzo[d][1,2,3]triazol-6-yl)-N-(2-(4-methylpiperazin-1-yl)pyridin-4-yl)-7H-pyrrolo[2,3-d]pyrimidin-2-amine [846];
5-(1-methyl-1H-benzo[d][1,2,3]triazol-6-yl)-N-(6-(4-methylpiperazin-1-yl)pyridin-3-yl)-7H-pyrrolo[2,3-d]pyrimidin-2-amine [847];
5-(1-methyl-1H-benzo[d][1,2,3]triazol-6-yl)-N-46-(4-methylpiperazin-1-yl)pyridin-3-yl)methyl)-7H-pyrrolo[2,3-d]pyrimidin-2-amine [848];
6-(5-(1-methyl-1H-benzo[d][1,2,3]triazol-6-yl)-7H-pyrrolo[2,3-d]pyrimidin-2-yl)quinoline [849];
1-methyl-6-(4-(1-methyl-1H-pyrazol-4-yl)-7H-pyrrolo[2,3-d]pyrimidin-5-yl)-1H-benzo[d][1,2,3]triazole [850];
5-(1-methyl-1H-benzo[d][1,2,3]triazol-6-yl)-N-(trans-4-morpholinocyclohexyl)-7H-pyrrolo[2,3-d]pyrimidin-4-amine [851];
1-ethyl-6-(2-isobutyl-7H-pyrrolo[2,3-d]pyrimidin-5-yl)-1H-benzo[d][1,2,3]triazole [853];
1-ethyl-6-(2-(3,3,3-trifluoropropyl)-7H-pyrrolo[2,3-d]pyrimidin-5-yl)-1H-benzo[d][1,2,3]triazole [854];
6-(2-cyclopropyl-7H-pyrrolo[2,3-d]pyrimidin-5-yl)-1-ethyl-1H-benzo[d][1,2,3]triazole [855];
6-(2-cyclobutyl-7H-pyrrolo[2,3-d]pyrimidin-5-yl)-1-ethyl-1H-benzo[d][1,2,3]triazole [856];
6-(2-(cyclobutylmethyl)-7H-pyrrolo[2,3-d]pyrimidin-5-yl)-1-ethyl-1H-benzo[d][1,2,3]triazole [857];
5-(1-ethyl-1H-benzo[d][1,2,3]triazol-6-yl)-7H-pyrrolo[2,3-d]pyrimidin-2-amine [858];
5-(1-ethyl-1H-benzo[d][1,2,3]triazol-6-yl)-N-methyl-7H-pyrrolo[2,3-d]pyrimidin-2-amine [859];
N-ethyl-5-(1-ethyl-1H-benzo[d][1,2,3]triazol-6-yl)-7H-pyrrolo[2,3-d]pyrimidin-2-amine [860];
5-(1-ethyl-1H-benzo[d][1,2,3]triazol-6-yl)-N-isopropyl-7H-pyrrolo[2,3-d]pyrimidin-2-amine [861];
5-(1-ethyl-1H-benzo[d][1,2,3]triazol-6-yl)-N-isobutyl-7H-pyrrolo[2,3-d]pyrimidin-2-amine [862];
5-(1-ethyl-1H-benzo[d][1,2,3]triazol-6-yl)-N-neopentyl-7H-pyrrolo[2,3-d]pyrimidin-2-amine [863];
(S)-N-(sec-butyl)-5-(1-ethyl-1H-benzo[d][1,2,3]triazol-6-yl)-7H-pyrrolo[2,3-d]pyrimidin-2-amine [864];
5-(1-ethyl-1H-benzo[d][1,2,3]triazol-6-yl)-N-(2-fluoro-2-methylpropyl)-7H-pyrrolo[2,3-d]pyrimidin-2-amine [865];
N-(2,2-difluoropropyl)-5-(1-ethyl-1H-benzo[d][1,2,3]triazol-6-yl)-7H-pyrrolo[2,3-d]pyrimidin-2-amine [866];
5-(1-ethyl-1H-benzo[d][1,2,3]triazol-6-yl)-N-(2,2,2-trifluoroethyl)-7H-pyrrolo[2,3-d]pyrimidin-2-amine [867];
(R)-5-(1-ethyl-1H-benzo[d][1,2,3]triazol-6-yl)-N-(1,1,1-trifluoropropan-2-yl)-7H-pyrrolo[2,3-d]pyrimidin-2-amine [868];
(S)-5-(1-ethyl-1H-benzo[d][1,2,3]triazol-6-yl)-N-(1,1,1-trifluoropropan-2-yl)-7H-pyrrolo[2,3-d]pyrimidin-2-amine [869];
5-(1-ethyl-1H-benzo[d][1,2,3]triazol-6-yl)-N-(3,3,3-trifluoropropyl)-7H-pyrrolo[2,3-d]pyrimidin-2-amine [870];
(S)-5-(1-ethyl-1H-benzo[d][1,2,3]triazol-6-yl)-N-(1-methoxypropan-2-yl)-7H-pyrrolo[2,3-d]pyrimidin-2-amine [871];
6-(2-ethoxy-7H-pyrrolo[2,3-d]pyrimidin-5-yl)-1-ethyl-1H-benzo[d][1,2,3]triazole [872];
5-(1-ethyl-1H-benzo[d][1,2,3]triazol-6-yl)-N-((1-fluorocyclopropyl)methyl)-7H-pyrrolo[2,3-d]pyrimidin-2-amine [873];
(R)-N-(1-cyclopropylethyl)-5-(1-ethyl-1H-benzo[d][1,2,3]triazol-6-yl)-7H-pyrrolo[2,3-d]pyrimidin-2-amine [874];
5-(1-ethyl-1H-benzo[d][1,2,3]triazol-6-yl)-N-((1-methylcyclopropyl)methyl)-7H-pyrrolo[2,3-d]pyrimidin-2-amine [875];
5-(1-ethyl-1H-benzo[d][1,2,3]triazol-6-yl)-N-41-(trifluoromethyl)cyclopropyl)methyl)-7H-pyrrolo[2,3-d]pyrimidin-2-amine [876];
N-(2-cyclopropyl-2,2-difluoroethyl)-5-(1-ethyl-1H-benzo[d][1,2,3]triazol-6-yl)-7H-pyrrolo[2,3-d]pyrimidin-2-amine [877];
N-(3,3-difluorocyclobutyl)-5-(1-ethyl-1H-benzo[d][1,2,3]triazol-6-yl)-7H-pyrrolo[2,3-d]pyrimidin-2-amine [878];
5-(1-ethyl-1H-benzo[d][1,2,3]triazol-6-yl)-N-((1-fluorocyclobutyl)methyl)-7H-pyrrolo[2,3-d]pyrimidin-2-amine [879];
N-(4,4-difluorocyclohexyl)-5-(1-ethyl-1H-benzo[d][1,2,3]triazol-6-yl)-7H-pyrrolo[2,3-d]pyrimidin-2-amine [880];
5-(1-ethyl-1H-benzo[d][1,2,3]triazol-6-yl)-N-(cis-4-methoxycyclohexyl)-7H-pyrrolo[2,3-d]pyrimidin-2-amine [881];
5-(1-ethyl-1H-benzo[d][1,2,3]triazol-6-yl)-N-((1-fluorocyclohexyl)methyl)-7H-pyrrolo[2,3-d]pyrimidin-2-amine [882];
N-((4,4-difluorocyclohexyl)methyl)-5-(1-ethyl-1H-benzo[d][1,2,3]triazol-6-yl)-7H-pyrrolo[2,3-d]pyrimidin-2-amine [883];
5-(1-ethyl-1H-benzo[d][1,2,3]triazol-6-yl)-N-(1-methylpiperidin-4-yl)-7H-pyrrolo[2,3-d]pyrimidin-2-amine [884];
5-(1-ethyl-1H-benzo[d][1,2,3]triazol-6-yl)-N-((1-methylpiperidin-4-yl)methyl)-7H-pyrrolo[2,3-d]pyrimidin-2-amine [885];
5-(1-ethyl-1H-benzo[d][1,2,3]triazol-6-yl)-N-(tetrahydro-2H-pyran-4-yl)-7H-pyrrolo[2,3-d]pyrimidin-2-amine [886];
5-(1-ethyl-1H-benzo[d][1,2,3]triazol-6-yl)-N-(3-(4-methylpiperazin-1-yl)phenyl)-7H-pyrrolo[2,3-d]pyrimidin-2-amine [887];
5-(1-ethyl-1H-benzo[d][1,2,3]triazol-6-yl)-N-(pyridin-4-yl)-7H-pyrrolo[2,3-d]pyrimidin-2-amine [888];
5-(1-ethyl-1H-benzo[d][1,2,3]triazol-6-yl)-N-(2-(4-methylpiperazin-1-yl)pyridin-4-yl)-7H-pyrrolo[2,3-d]pyrimidin-2-amine [889];
5-(1-ethyl-1H-benzo[d][1,2,3]triazol-6-yl)-N-(6-(4-methylpiperazin-1-yl)pyridin-3-yl)-7H-pyrrolo[2,3-d]pyrimidin-2-amine [890];
5-(1-ethyl-1H-benzo[d][1,2,3]triazol-6-yl)-N-46-(4-methylpiperazin-1-yl)pyridin-3-yl)methyl)-7H-pyrrolo[2,3-d]pyrimidin-2-amine [891];
6-(5-(1-ethyl-1H-benzo[d][1,2,3]triazol-6-yl)-7H-pyrrolo[2,3-d]pyrimidin-2-yl)quinoline [892];
1-ethyl-6-(4-(1-methyl-1H-pyrazol-4-yl)-7H-pyrrolo[2,3-d]pyrimidin-5-yl)-1H-benzo[d][1,2,3]triazole [893];
5-(1-ethyl-1H-benzo[d][1,2,3]triazol-6-yl)-N-(trans-4-morpholinocyclohexyl)-7H-pyrrolo[2,3-d]pyrimidin-4-amine [894];
N-(2-fluoro-2-methylpropyl)-5-(3-methyl-[1,2,4]triazolo [4,3-a]pyridin-6-yl)-7H-pyrrolo [2,3-d]pyrimidin-2-amine [895];
5-(imidazo [1,2-b]pyridazin-6-yl)-2-isobutyl-7H-pyrrolo [2,3-d] pyrimidine [897];
5-(imidazo [1,2-b]pyridazin-6-yl)-2-(3,3,3-trifluoropropyl)-7H-pyrrolo [2,3-d] pyrimidine [898];
2-cyclopropyl-5-(imidazo [1,2-b] pyridazin-6-yl)-7H-pyrrolo [2,3-d] pyrimidine [899];
2-cyclobutyl-5-(imidazo [1,2-b]pyridazin-6-yl)-7H-pyrrolo [2,3-d]pyrimidine [900];
2-(cyclobutylmethyl)-5-(imidazo [1,2-b]pyridazin-6-yl)-7H-pyrrolo [2,3-d]pyrimidine [901];
5-(imidazo [1,2-b]pyridazin-6-yl)-7H-pyrrolo [2,3-d]pyrimidin-2-amine [902];
5-(imidazo [1,2-b]pyridazin-6-yl)-N-methyl-7H-pyrrolo [2,3-d] pyrimidin-2-amine [903];
N-ethyl-5-(imidazo [1,2-b] pyridazin-6-yl)-7H-pyrrolo [2,3-d]pyrimidin-2-amine [904];
5-(imidazo [1,2-b]pyridazin-6-yl)-N-i sopropyl-7H-pyrrolo [2,3-d]pyrimidin-2-amine [905];
5-(imidazo [1,2-b]pyridazin-6-yl)-N-i sobutyl-7H-pyrrolo [2,3-d]pyrimidin-2-amine [906];
5-(imidazo [1,2-b]pyridazin-6-yl)-N-ne opentyl-7H-pyrrolo [2,3-d]pyrimidin-2-amine [907];
(S)-N-(sec-butyl)-5-(imidazo [1,2-b] pyridazin-6-yl)-7H-pyrrolo [2,3-d]pyrimidin-2-amine [908];
N-(2-fluoro-2-methylpropyl)-5-(imidazo [1,2-b]pyridazin-6-yl)-7H-pyrrolo [2,3-d]pyrimidin-2-amine [909];
N-(2,2-difluoropropyl)-5-(imidazo [1,2-b]pyridazin-6-yl)-7H-pyrrolo [2,3-d] pyrimidin-2-amine [910];
5-(imidazo [1,2-b]pyridazin-6-yl)-N-(2,2,2-trifluoroethyl)-7H-pyrrolo [2,3-d]pyrimidin-2-amine [911];
(R)-5-(imidazo [1,2-b] pyridazin-6-yl)-N-(1,1,1-trifluoropropan-2-yl)-7H-pyrrolo [2,3-d]pyrimidin-2-amine [912];
(S)-5-(imidazo [1,2-b] pyridazin-6-yl)-N-(1,1,1-trifluoropropan-2-yl)-7H-pyrrolo [2,3-d]pyrimidin-2-amine [913];
5-(imidazo [1,2-b]pyridazin-6-yl)-N-(3,3,3-trifluoropropyl)-7H-pyrrolo [2,3-d]pyrimidin-2-amine ;
(S)-5-(imidazo [1,2-b] pyridazin-6-yl)-N-(1-methoxypropan-2-yl)-7H-pyrrolo [2,3-d]pyrimidin-2-amine [915];
2-ethoxy-5-(imidazo [1,2-b]pyridazin-6-yl)-7H-pyrrolo [2,3-d] pyrimidine [916];
N-((1-fluorocyclopropyl)methyl)-5-(imidazo [1,2-b] pyridazin-6-yl)-7H-pyrrolo [2,3-d] pyrimidin-2-amine [917];
(R)-N-(1-cyclopropylethyl)-5-(imidazo [1,2-b] pyridazin-6-yl)-7H-pyrrolo [2,3-d] pyrimidin-2-amine [918];
5-(imidazo[1,2-b]pyridazin-6-yl)-N-((1-methylcyclopropyl)methyl)-7H-pyrrolo[2,3-d]pyrimidin-2-amine [919];
5-(imidazo[1,2-b]pyridazin-6-yl)-N-41-(trifluoromethyl)cyclopropyl)methyl)-7H-pyrrolo[2,3-d]pyrimidin-2-amine [920];
N-(2-cyclopropyl-2,2-difluoroethyl)-5-(imidazo[1,2-b]pyridazin-6-yl)-7H-pyrrolo[2,3-d]pyrimidin-2-amine [921];
N-(3,3-difluorocyclobutyl)-5-(imidazo[1,2-b]pyridazin-6-yl)-7H-pyrrolo[2,3-d]pyrimidin-2-amine [922];
N-((1-fluorocyclobutyl)methyl)-5-(imidazo[1,2-b]pyridazin-6-yl)-7H-pyrrolo[2,3-d]pyrimidin-2-amine [923];
N-(4,4-difluorocyclohexyl)-5-(imidazo[1,2-b]pyridazin-6-yl)-7H-pyrrolo[2,3-d]pyrimidin-2-amine [924];
5-(imidazo[1,2-b]pyridazin-6-yl)-N-(cis-4-methoxycyclohexyl)-7H-pyrrolo[2,3-d]pyrimidin-2-amine [925];
N-((1-fluorocyclohexyl)methyl)-5-(imidazo[1,2-b]pyridazin-6-yl)-7H-pyrrolo[2,3-d]pyrimidin-2-amine [926];
N-((4,4-difluorocyclohexyl)methyl)-5-(imidazo[1,2-b]pyridazin-6-yl)-7H-pyrrolo[2,3-d]pyrimidin-2-amine [927];
5-(imidazo[1,2-b]pyridazin-6-yl)-N-(1-methylpiperidin-4-yl)-7H-pyrrolo[2,3-d]pyrimidin-2-amine [928];
5-(imidazo[1,2-b]pyridazin-6-yl)-N-((1-methylpiperidin-4-yl)methyl)-7H-pyrrolo[2,3-d]pyrimidin-2-amine [929];
5-(imidazo[1,2-b]pyridazin-6-yl)-N-(tetrahydro-2H-pyran-4-yl)-7H-pyrrolo[2,3-d]pyrimidin-2-amine [930];
5-(imidazo[1,2-b]pyridazin-6-yl)-N-(3-(4-methylpiperazin-1-yl)phenyl)-7H-pyrrolo[2,3-d]pyrimidin-2-amine [931];
5-(imidazo[1,2-b]pyridazin-6-yl)-N-(pyridin-4-yl)-7H-pyrrolo[2,3-d]pyrimidin-2-amine [932];
5-(imidazo[1,2-b]pyridazin-6-yl)-N-(2-(4-methylpiperazin-1-yl)pyridin-4-yl)-7H-pyrrolo[2,3-d]pyrimidin-2-amine [933];
5-(imidazo[1,2-b]pyridazin-6-yl)-N-(6-(4-methylpiperazin-1-yl)pyridin-3-yl)-7H-pyrrolo[2,3-d]pyrimidin-2-amine [934];
5-(imidazo[1,2-b]pyridazin-6-yl)-N-46-(4-methylpiperazin-1-yl)pyridin-3-yl)methyl)-7H-pyrrolo[2,3-d]pyrimidin-2-amine [935];
6-(5-(imidazo[1,2-b]pyridazin-6-yl)-7H-pyrrolo[2,3-d]pyrimidin-2-yl)quinoline [936];
5-(imidazo [1,2-b]pyridazin-6-yl)-4-(1-methyl-1H-pyrazol-4-yl)-7H-pyrrolo [2,3-d]pyrimidine [937];
5-(imidazo [1,2-b]pyridazin-6-yl)-N-(trans-4-morpholinocyclohexyl)-7H-pyrrolo [2,3-d]pyrimidin-4-amine [938];
5-(3-chloroimidazo [1,2-b]pyridazin-6-yl)-2-isobutyl-7H-pyrrolo [2,3-d]pyrimidine [940];
5-(3-chloroimidazo [1,2-b]pyridazin-6-yl)-2-(3,3,3-trifluoropropyl)-7H-pyrrolo [2,3-d]pyrimidine [941];
5-(3-chloroimidazo [1,2-b]pyridazin-6-yl)-2-cyclopropyl-7H-pyrrolo [2,3-d]pyrimidine [942];
5-(3-chloroimidazo [1,2-b]pyridazin-6-yl)-2-cyclobutyl-7H-pyrrolo [2,3-d]pyrimidine [943];
5-(3-chloroimidazo [1,2-b]pyridazin-6-yl)-2-(cyclobutylmethyl)-7H-pyrrolo [2,3-d]pyrimidine [944];
5-(3-chloroimidazo [1,2-b]pyridazin-6-yl)-7H-pyrrolo [2,3-d]pyrimidin-2-amine [945];
5-(3-chloroimidazo [1,2-b] pyridazin-6-yl)-N-methyl-7H-pyrrolo [2,3-d] pyrimidin-2-amine [946];
5-(3-chloroimidazo [1,2-b] pyridazin-6-yl)-N-ethyl-7H-pyrrolo [2,3-d] pyrimidin-2-amine [947];
5-(3-chloroimidazo [1,2-b]pyridazin-6-yl)-N-isopropyl-7H-pyrrolo [2,3-d] pyrimidin-2-amine [948];
5-(3-chloroimidazo [1,2-b]pyridazin-6-yl)-N-isobutyl-7H-pyrrolo [2,3-d]pyrimidin-2-amine [949];
5-(3-chloroimidazo [1,2-b]pyridazin-6-yl)-N-neopentyl-7H-pyrrolo [2,3-d]pyrimidin-2-amine [950];
(S)-N-(sec-butyl)-5-(3-chloroimidazo [1,2-b]pyridazin-6-yl)-7H-pyrrolo [2,3-d]pyrimidin-2-amine [951];
5-(3-chloroimidazo [1,2-b]pyridazin-6-yl)-N-(2-fluoro-2-methylpropyl)-7H-pyrrolo [2,3-d]pyrimidin-2-amine [952];
5-(3-chloroimidazo [1,2-b]pyridazin-6-yl)-N-(2,2-difluoropropyl)-7H-pyrrolo [2,3-d] pyrimidin-2-amine [953];
5-(3-chloroimidazo [1,2-b]pyridazin-6-yl)-N-(2,2,2-trifluoroethyl)-7H-pyrrolo [2,3-d]pyrimidin-2-amine [954];
(R)-5-(3-chloroimidazo [1,2-b]pyridazin-6-yl)-N-(1,1,1-trifluoropropan-2-yl)-7H-pyrrolo [2,3-d]pyrimidin-2-amine [955];
(S)-5-(3-chloroimidazo[1,2-b]pyridazin-6-yl)-N-(1,1,1-trifluoropropan-2-yl)-7H-pyrrolo [2,3-d]pyrimidin-2-amine [956];
5-(3-chloroimidazo [1,2-b]pyridazin-6-yl)-N-(3,3,3-trifluoropropyl)-7H-pyrrolo [2,3-d]pyrimidin-2-amine [957];
(S)-5-(3-chloroimidazo [1,2-b] pyridazin-6-yl)-N-(1-methoxypropan-2-yl)-7H-pyrrolo [2,3-d]pyrimidin-2-amine [958];
5-(3-chloroimidazo [1,2-b] pyridazin-6-yl)-2-ethoxy-7H-pyrrolo [2,3-d] pyrimidine [959];
5-(3-chloroimidazo [1,2-b] pyridazin-6-yl)-N-((1-fluorocyclopropyl)methyl)-7H-pyrrolo [2,3-d]pyrimidin-2-amine [960];
(R)-5-(3-chloroimidazo [1,2-b]pyridazin-6-yl)-N-(1-cyclopropylethyl)-7H-pyrrolo [2,3-d]pyrimidin-2-amine [961];
5-(3-chloroimidazo [1,2-b] pyridazin-6-yl)-N-((1-methylcyclopropyl)methyl)-7H-pyrrolo [2,3-d]pyrimidin-2-amine [962];
5-(3-chloroimidazo [1,2-b] pyridazin-6-yl)-N-41-(trifluoromethyl)cyclopropyl)methyl)-7H-pyrrolo [2,3-d]pyrimidin-2-amine [963];
5-(3-chloroimidazo [1,2-b] pyridazin-6-yl)-N-(2-cyclopropyl-2,2-difluoroethyl)-7H-pyrrolo [2,3-d]pyrimidin-2-amine [964];
5-(3-chloroimidazo [1,2-b] pyridazin-6-yl)-N-(3,3-difluorocyclobutyl)-7H-pyrrolo [2,3-d]pyrimidin-2-amine [965];
5-(3-chloroimidazo [1,2-b] pyridazin-6-yl)-N-((1-fluorocyclobutypmethyl)-7H-pyrrolo [2,3-d]pyrimidin-2-amine [966];
5-(3-chloroimidazo [1,2-b] pyridazin-6-yl)-N-(4,4-difluorocyclohexyl)-7H-pyrrolo [2,3-d]pyrimidin-2-amine [967];
5-(3-chloroimidazo [1,2-b] pyridazin-6-yl)-N-(cis-4-methoxycyclohexyl)-7H-pyrrolo [2,3-d]pyrimidin-2-amine [968];
5-(3-chloroimidazo [1,2-b] pyridazin-6-yl)-N-((1-fluorocyclohexyl)methyl)-7H-pyrrolo [2,3-d]pyrimidin-2-amine [969];
5-(3-chloroimidazo [1,2-b] pyridazin-6-yl)-N-((4,4-difluorocyclohexyl)methyl)-7H-pyrrolo [2,3-d]pyrimidin-2-amine [970];
5-(3-chloroimidazo [1,2-b] pyridazin-6-yl)-N-(1-methylpiperidin-4-yl)-7H-pyrrolo [2,3-d]pyrimidin-2-amine [971];
5-(3-chloroimidazo [1,2-b] pyridazin-6-yl)-N-((1-methylpiperidin-4-yl)methyl)-7H-pyrrolo [2,3-d]pyrimidin-2-amine [972];
5-(3-chloroimidazo [1,2-b] pyridazin-6-yl)-N-(tetrahydro -2H-pyran-4-yl)-7H-pyrrolo [2,3-d]pyrimidin-2-amine [973];
5-(3-chloroimidazo [1,2-b] pyridazin-6-yl)-N-(3-(4-methylpipe razin-1-yl)phenyl)-7H-pyrrolo [2,3-d]pyrimidin-2-amine [974];
5-(3-chloroimidazo [1,2-b]pyridazin-6-yl)-N-(pyridin-4-yl)-7H-pyrrolo [2,3-d]pyrimidin-2-amine [975];
5-(3-chloroimidazo [1,2-b]pyridazin-6-yl)-N-(2-(4-methylpiperazin-1-yl)pyridin-4-yl)-7H-pyrrolo [2,3-d]pyrimidin-2-amine [976];
5-(3-chloroimidazo [1,2-b]pyridazin-6-yl)-N-(6-(4-methylpiperazin-1-yl)pyridin-3-yl)-7H-pyrrolo [2,3-d]pyrimidin-2-amine [977];
5-(3-chloroimidazo [1,2-b]pyridazin-6-yl)-N-46-(4-me thylpiperazin-1-yl)pyridin-3-yl)methyl)-7H-pyrrolo [2,3-d]pyrimidin-2-amine [978];
6-(5-(3-chloroimidazo [1,2-b]pyridazin-6-yl)-7H-pyrrolo [2,3-d]pyrimidin-2-yl)quinoline [979];
5-(3-chloroimidazo [1,2-b]pyridazin-6-yl)-4-(1-methyl-1H-pyrazol-4-yl)-7H-pyrrolo [2,3-d]pyrimidine [980];
5-(3-chloroimidazo [1,2-b]pyridazin-6-yl)-N-(trans-4-morpholinocyclohexyl)-7H-pyrrolo [2,3-d]pyrimidin-4-amine [981];
2-i sobutyl-5-(2-methylimidazo [1,2-b]pyridazin-6-yl)-7H-pyrrolo [2,3-d] pyrimidine [983];
5-(2-methylimidazo [1,2-b]pyridazin-6-yl)-2-(3,3,3-trifluoropropyl)-7H-pyrrolo [2,3-d]pyrimidine [984];
2-cyclopropyl-5-(2-methylimidazo [1,2-b]pyridazin-6-yl)-7H-pyrrolo [2,3-d]pyrimidine [985];
2-cyclobutyl-5-(2-methylimidazo [1,2-b]pyridazin-6-yl)-7H-pyrrolo [2,3-d]pyrimidine [986];
2-(cyclobutylmethyl)-5-(2-methylimidazo [1,2-b]pyridazin-6-yl)-7H-pyrrolo [2,3-d] pyrimidine [987];
5-(2-methylimidazo [1,2-b]pyridazin-6-yl)-7H-pyrrolo [2,3-d]pyrimidin-2-amine [988];
N-methyl-5-(2-methylimidazo [1,2-b]pyridazin-6-yl)-7H-pyrrolo [2,3-d]pyrimidin-2-amine [989];
N-ethyl-5-(2-methylimidazo [1,2-b]pyridazin-6-yl)-7H-pyrrolo [2,3-d]pyrimidin-2-amine [990];
N-isopropyl-5-(2-methylimidazo [1,2-b]pyridazin-6-yl)-7H-pyrrolo [2,3-d]pyrimidin-2-amine [991];
N-isobutyl-5-(2-methylimidazo [1,2-b]pyridazin-6-yl)-7H-pyrrolo [2,3-d]pyrimidin-2-amine [992];
5-(2-methylimidazo [1,2-b]pyridazin-6-yl)-N-neopentyl-7H-pyrrolo [2,3-d] pyrimidin-2-amine [993];
(S)-N-(sec-butyl)-5-(2-methylimidazo [1,2-b]pyridazin-6-yl)-7H-pyrrolo [2,3-d]pyrimidin-2-amine [994];
N-(2-fluoro-2-methylpropyl)-5-(2-methylimidazo [1,2-b]pyridazin-6-yl)-7H-pyrrolo [2,3-d]pyrimidin-2-amine [995];
N-(2,2-difluoropropyl)-5-(2-methylimidazo [1,2-b]pyridazin-6-yl)-7H-pyrrolo [2,3-d] pyrimidin-2-amine [996];
5-(2-methylimidazo [1,2-b]pyridazin-6-yl)-N-(2,2,2-trifluoroethyl)-7H-pyrrolo [2,3-d]pyrimidin-2-amine [997];
(R)-5-(2-methylimidazo [1,2-b]pyridazin-6-yl)-N-(1,1,1-trifluoropropan-2-yl)-7H-pyrrolo [2,3-d]pyrimidin-2-amine [998];
(S)-5-(2-methylimidazo [1,2-b]pyridazin-6-yl)-N-(1,1,1-trifluoropropan-2-yl)-7H-pyrrolo [2,3-d]pyrimidin-2-amine [999]; and
5-(2-methylimidazo [1,2-b]pyridazin-6-yl)-N-(3,3,3-trifluoropropyl)-7H-pyrrolo [2,3-d]pyrimidin-2-amine [1000]; or a pharmaceutically acceptable salt thereof.

25. The compound of claim 1, wherein the compound of Formula I is selected from the group consisting of:
(S)-N-(1-methoxypropan-2-yl)-5-(2-methylimidazo [1,2-b]pyridazin-6-yl)-7H-pyrrolo [2,3-d]pyrimidin-2-amine [1001];
2-ethoxy-5-(2-methylimidazo[1,2-b]pyridazin-6-yl)-7H-pyrrolo[2,3-d]pyrimidine [1002];
N-((1-fluorocyclopropyl)methyl)-5-(2-methylimidazo [1,2-b]pyridazin-6-yl)-7H-pyrrolo [2,3-d]pyrimidin-2-amine [1003];
(R)-N-(1-cyclopropylethyl)-5-(2-methylimidazo [1,2-b]pyridazin-6-yl)-7H-pyrrolo [2,3-d]pyrimidin-2-amine [1004];
N-((1-methylcyclopropyl)methyl)-5-(2-methylimidazo [1,2-b]pyridazin-6-yl)-7H-pyrrolo [2,3-d]pyrimidin-2-amine [1005];
5-(2-methylimidazo [1,2-b] pyridazin-6-yl)-N-41-(trifluoromethyl)cyclopropyl)me thyl) -7H-pyrrolo [2,3-d]pyrimidin-2-amine [1006];
N-(2-cyclopropyl-2,2-difluoroethyl)-5-(2-methylimidazo [1,2-b]pyridazin-6-yl)-7H-pyrrolo [2,3-d]pyrimidin-2-amine [1007];
N-(3,3-difluorocyclobutyl)-5-(2-methylimidazo [1,2-b]pyridazin-6-yl)-7H-pyrrolo [2,3-d]pyrimidin-2-amine [1008];
N-((1-fluorocyclobutypmethyl)-5-(2-methylimidazo [1,2-b]pyridazin-6-yl)-7H-pyrrolo [2,3-d]pyrimidin-2-amine [1009];
N-(4,4-difluorocyclohexyl)-5-(2-methylimidazo [1,2-b]pyridazin-6-yl)-7H-pyrrolo [2,3-d]pyrimidin-2-amine [1010];
N-(cis-4-methoxycyclohexyl)-5-(2-methylimidazo [1,2-b]pyridazin-6-yl)-7H-pyrrolo [2,3-d]pyrimidin-2-amine [1011];
N-((1-fluorocyclohexyl)methyl)-5-(2-methylimidazo [1,2-b]pyridazin-6-yl)-7H-pyrrolo [2,3-d]pyrimidin-2-amine [1012];
N-((4,4-difluorocyclohexyl)methyl)-5-(2-methylimidazo [1,2-b]pyridazin-6-yl)-7H-pyrrolo [2,3-d]pyrimidin-2-amine [1013];
5-(2-methylimidazo [1,2-b]pyridazin-6-yl)-N-(1-methylpiperidin-4-yl)-7H-pyrrolo [2,3-d]pyrimidin-2-amine [1014];
5-(2-methylimidazo [1,2-b]pyridazin-6-yl)-N-((1-methylpiperidin-4-yl)methyl)-7H-pyrrolo [2,3-d]pyrimidin-2-amine [1015];
5-(2-methylimidazo [1,2-b]pyridazin-6-yl)-N-(tetrahydro-2H-pyran-4-yl)-7H-pyrrolo [2,3-d]pyrimidin-2-amine [1016];
5-(2-methylimidazo [1,2-b]pyridazin-6-yl)-N-(3-(4-methylpiperazin-1-yl)phenyl)-7H-pyrrolo [2,3-d]pyrimidin-2-amine [1017];
5-(2-methylimidazo [1,2-b]pyridazin-6-yl)-N-(pyridin-4-yl)-7H-pyrrolo [2,3-d] pyrimidin-2-amine [1018];
5-(2-methylimidazo [1,2-b]pyridazin-6-yl)-N-(2-(4-methylpiperazin-1-yl)pyridin-4-yl)-7H-pyrrolo [2,3-d]pyrimidin-2-amine [1019];
5-(2-methylimidazo [1,2-b]pyridazin-6-yl)-N-(6-(4-methylpiperazin-1-yl)pyridin-3-yl)-7H-pyrrolo [2,3-d]pyrimidin-2-amine [1020];
5-(2-methylimidazo [1,2-b]pyridazin-6-yl)-N-46-(4-methylpiperazin-1-yl)pyridin-3-yl)methyl)-7H-pyrrolo [2,3-d]pyrimidin-2-amine [1021];
6-(5-(2-methylimidazo [1,2-b]pyridazin-6-yl)-7H-pyrrolo [2,3-d]pyrimidin-2-yl)quinoline [1022];
4-(1-methyl-1H-pyrazol-4-yl)-5-(2-methylimidazo [1,2-b]pyridazin-6-yl)-7H-pyrrolo [2,3-d]pyrimidine [1023];
5-(2-methylimidazo [1,2-b] pyridazin-6-yl)-N-(trans-4-morpho linocyclohexyl)-7H-pyrrolo [2,3-d]pyrimidin-4-amine [1024];
2-i sobutyl-5-(3-methylimidazo [1,2-b]pyridazin-6-yl)-7H-pyrrolo [2,3-d] pyrimidine [1026];
5-(3-methylimidazo [1,2-b]pyridazin-6-yl)-2-(3,3,3-trifluoropropyl)-7H-pyrrolo [2,3-d]pyrimidine [1027];
2-cyclopropyl-5-(3-methylimidazo [1,2-b]pyridazin-6-yl)-7H-pyrrolo [2,3-d]pyrimidine [1028];
2-cyclobutyl-5-(3-methylimidazo [1,2-b]pyridazin-6-yl)-7H-pyrrolo [2,3-d]pyrimidine [1029];
2-(cyclobutylmethyl)-5-(3-methylimidazo [1,2-b]pyridazin-6-yl)-7H-pyrrolo [2,3-d] pyrimidine [1030];
5-(3-methylimidazo [1,2-b]pyridazin-6-yl)-7H-pyrrolo [2,3-d]pyrimidin-2-amine [1031];
N-methyl-5-(3-methylimidazo [1,2-b]pyridazin-6-yl)-7H-pyrrolo [2,3-d]pyrimidin-2-amine [1032];
N-ethyl-5-(3-methylimidazo [1,2-b]pyridazin-6-yl)-7H-pyrrolo [2,3-d]pyrimidin-2-amine [1033];
N-isopropyl-5-(3-methylimidazo [1,2-b] pyridazin-6-yl)-7H-pyrrolo [2,3-d]pyrimidin-2-amine [1034];
N-isobutyl-5-(3-methylimidazo [1,2-b]pyridazin-6-yl)-7H-pyrrolo [2,3-d]pyrimidin-2-amine [1035];
5-(3-methylimidazo [1,2-b] pyridazin-6-yl)-N-neopentyl-7H-pyrrolo [2,3-d] pyrimidin-2-amine [1036];
(S)-N-(sec-butyl)-5-(3-methylimidazo [1,2-b]pyridazin-6-yl)-7H-pyrrolo [2,3-d]pyrimidin-2-amine [1037];
N-(2-fluoro-2-methylpropyl)-5-(3-methylimidazo [1,2-b] pyridazin-6-yl)-7H-pyrrolo [2,3-d]pyrimidin-2-amine [1038];
N-(2,2-difluoropropyl)-5-(3-methylimidazo [1,2-b] pyridazin-6-yl)-7H-pyrrolo [2,3-d] pyrimidin-2-amine [1039];
5-(3-methylimidazo [1,2-b] pyridazin-6-yl)-N-(2,2,2-trifluoroethyl)-7H-pyrrolo [2,3-d] pyrimidin-2-amine [1040];
(R)-5-(3-methylimidazo [1,2-b]pyridazin-6-yl)-N-(1,1,1-trifluoropropan-2-yl)-7H-pyrrolo [2,3-d]pyrimidin-2-amine [1041];
(S)-5-(3-methylimidazo [1,2-b] pyridazin-6-yl)-N-(1,1,1-trifluoropropan-2-yl)-7H-pyrrolo [2,3-d]pyrimidin-2-amine [1042];
5-(3-methylimidazo [1,2-b] pyridazin-6-yl)-N-(3,3,3-trifluoropropyl)-7H-pyrrolo [2,3-d] pyrimidin-2-amine [1043];
(S)-N-(1-methoxypropan-2-yl)-5-(3-methylimidazo [1,2-b]pyridazin-6-yl)-7H-pyrrolo [2,3-d]pyrimidin-2-amine [1044];
2-ethoxy-5-(3-methylimidazo [1,2-b]pyridazin-6-yl)-7H-pyrrolo [2,3-d] pyrimidine [1045];
N-((1-fluorocyclopropyl)methyl)-5-(3-methylimidazo [1,2-b]pyridazin-6-yl)-7H-pyrrolo [2,3-d]pyrimidin-2-amine [1046];
(R)-N-(1-cyclopropylethyl)-5-(3-methylimidazo [1,2-b]pyridazin-6-yl)-7H-pyrrolo [2,3-d]pyrimidin-2-amine [1047];
N-((l-methylcyclopropyl)methyl)-5-(3-methylimidazo [1,2-b]pyridazin-6-yl)-7H-pyrrolo [2,3-d]pyrimidin-2-amine [1048];
5-(3-methylimidazo [1,2-b]pyridazin-6-yl)-N-41-(trifluoromethyl)cyclopropyl)me thyl)-7H-pyrrolo [2,3-d]pyrimidin-2-amine [1049];
N-(2-cyclopropyl-2,2-difluoroethyl)-5-(3-methylimidazo [1,2-b]pyridazin-6-yl)-7H-pyrrolo [2,3-d]pyrimidin-2-amine [1050];
N-(3,3-difluorocyclobutyl)-5-(3-methylimidazo [1,2-b]pyridazin-6-yl)-7H-pyrrolo [2,3-d]pyrimidin-2-amine [1051];
N-((1-fluorocyclobutypmethyl)-5-(3-methylimidazo [1,2-b]pyridazin-6-yl)-7H-pyrrolo [2,3-d]pyrimidin-2-amine [1052];
N-(4,4-difluorocyclohexyl)-5-(3-methylimidazo [1,2-b]pyridazin-6-yl)-7H-pyrrolo [2,3-d]pyrimidin-2-amine [1053];
N-(cis-4-methoxycyclohexyl)-5-(3-methylimidazo [1,2-b]pyridazin-6-yl)-7H-pyrrolo [2,3-d]pyrimidin-2-amine [1054];
N-((1-fluorocyclohexyl)methyl)-5-(3-methylimidazo [1,2-b]pyridazin-6-yl)-7H-pyrrolo [2,3-d]pyrimidin-2-amine [1055];
N-((4,4-difluorocyclohexyl)methyl)-5-(3-methylimidazo [1,2-b]pyridazin-6-yl)-7H-pyrrolo [2,3-d]pyrimidin-2-amine [1056];
5-(3-methylimidazo [1,2-b]pyridazin-6-yl)-N-(1-methylpiperidin-4-yl)-7H-pyrrolo [2,3-d]pyrimidin-2-amine [1057];
5-(3-methylimidazo [1,2-b]pyridazin-6-yl)-N-((1-methylpiperidin-4-yl)methyl)-7H-pyrrolo [2,3-d]pyrimidin-2-amine [1058];
5-(3-methylimidazo [1,2-b]pyridazin-6-yl)-N-(tetrahydro-2H-pyran-4-yl)-7H-pyrrolo [2,3-d]pyrimidin-2-amine [1059];
5-(3-methylimidazo [1,2-b]pyridazin-6-yl)-N-(3-(4-methylpiperazin-1-yl)phenyl)-7H-pyrrolo [2,3-d]pyrimidin-2-amine [1060];
5-(3-methylimidazo [1,2-b]pyridazin-6-yl)-N-(pyridin-4-yl)-7H-pyrrolo [2,3-d] pyrimidin-2-amine [1061];
5-(3-methylimidazo [1,2-b]pyridazin-6-yl)-N-(2-(4-methylpiperazin-1-yl)pyridin-4-yl)-7H-pyrrolo [2,3-d]pyrimidin-2-amine [1062];
5-(3-methylimidazo [1,2-b]pyridazin-6-yl)-N-(6-(4-methylpiperazin-1-yl)pyridin-3-yl)-7H-pyrrolo [2,3-d]pyrimidin-2-amine [1063];
5-(3-methylimidazo [1,2-b]pyridazin-6-yl)-N-46-(4-methylpiperazin-1-yl)pyridin-3-yl)methyl)-7H-pyrrolo [2,3-d]pyrimidin-2-amine [1064];
6-(5-(3-methylimidazo [1,2-b]pyridazin-6-yl)-7H-pyrrolo [2,3-d]pyrimidin-2-yl)quinoline [1065];
4-(1-methyl-1H-pyrazol-4-yl)-5-(3-methylimidazo [1,2-b]pyridazin-6-yl)-7H-pyrrolo [2,3-d]pyrimidine [1066];
5-(3-methylimidazo [1,2-b]pyridazin-6-yl)-N-(trans-4-morpholinocyclohexyl)-7H-pyrrolo [2,3-d]pyrimidin-4-amine [1067];
5-(3-(difluoromethyl)imidazo[1,2-b]pyridazin-6-yl)-2-isobutyl-7H-pyrrolo [2,3-d]pyrimidine [1069];
5-(3-(difluoromethyl)imidazo[1,2-b]pyridazin-6-yl)-2-(3 ,3 ,3-trifluoropropyl)-7H-pyrrolo [2,3-d]pyrimidine [1070];
2-cyclopropyl-5-(3-(difluoromethyl)imidazo[1,2-b]pyridazin-6-yl)-7H-pyrrolo [2,3-d]pyrimidine [1071];
2-cyclobutyl-5-(3-(difluoromethyl)imidazo[1,2-b]pyridazin-6-yl)-7H-pyrrolo [2,3-d] pyrimidine [1072];
2-(cyclobutylmethyl)-5-(3-(difluoromethyl)imidazo[1,2-b]pyridazin-6-yl)-7H-pyrrolo [2,3-d]pyrimidine [1073];
5-(3-(difluoromethyl)imidazo[1,2-b]pyridazin-6-yl)-7H-pyrrolo [2,3-d] pyrimidin-2-amine [1074];
5-(3-(difluoromethyl)imidazo[1,2-b]pyridazin-6-yl)-N-methyl-7H-pyrrolo [2,3-d]pyrimidin-2-amine [1075];
5-(3-(difluoromethyl)imidazo[1,2-b]pyridazin-6-yl)-N-ethyl-7H-pyrrolo [2,3-d]pyrimidin-2-amine [1076];
5-(3-(difluoromethyl)imidazo[1,2-b]pyridazin-6-yl)-N-i sopropyl-7H-pyrrolo [2,3-d]pyrimidin-2-amine [1077];
5-(3-(difluoromethyl)imidazo[1,2-b]pyridazin-6-yl)-N-i sobutyl-7H-pyrrolo [2,3-d]pyrimidin-2-amine [1078];
5-(3-(difluoromethyl)imidazo[1,2-b]pyridazin-6-yl)-N-neopentyl-7H-pyrrolo [2,3-d]pyrimidin-2-amine [1079];
(S)-N-(sec-butyl)-5-(3-(difluoromethyl)imidazo [1,2-b]pyridazin-6-yl)-7H-pyrrolo [2,3-d]pyrimidin-2-amine [1080];
5-(3-(difluoromethyl)imidazo[1,2-b]pyridazin-6-yl)-N-(2-fluoro-2-me thylpropyl)-7H-pyrrolo [2,3-d]pyrimidin-2-amine [1081];
5-(3-(difluoromethyl)imidazo[1,2-b]pyridazin-6-yl)-N-(2,2-difluoropropyl)-7H-pyrrolo [2,3-d]pyrimidin-2-amine [1082];
5-(3-(difluoromethyl)imidazo[1,2-b]pyridazin-6-yl)-N-(2,2,2-trifluoroethyl)-7H-pyrrolo [2,3-d]pyrimidin-2-amine [1083];
(R)-5-(3-(difluoromethyl)imidazo [1,2-b]pyridazin-6-yl)-N-(1, 1, 1-trifluoropropan-2-yl)-7H-pyrrolo [2,3-d]pyrimidin-2-amine [1084];
(S)-5-(3-(difluoromethyl)imidazo[1,2-b]pyridazin-6-yl)-N-(1, 1, 1-trifluoropropan-2-yl)-7H-pyrrolo [2,3-d]pyrimidin-2-amine [1085];
5-(3-(difluoromethyl)imidazo[1,2-b]pyridazin-6-yl)-N-(3 ,3 ,3-trifluoropropyl)-7H-pyrrolo [2,3-d]pyrimidin-2-amine [1086];
(S)-5-(3-(difluoromethyl)imidazo[1,2-b]pyridazin-6-yl)-N-(1-methoxypropan-2-yl)-7H-pyrrolo [2,3-d]pyrimidin-2-amine [1087];
5-(3-(difluoromethyl)imidazo[1,2-b]pyridazin-6-yl)-2-ethoxy-7H-pyrrolo [2,3-d]pyrimidine ;
5-(3-(difluoromethyl)imidazo[1,2-b] pyridazin-6-yl)-N-((1-fluorocyclopropyl)me thyl)-7H-pyrrolo [2,3-d]pyrimidin-2-amine [1089];
(R)-N-(1-cyclopropylethyl)-5-(3-(difluoromethyl)imidazo [1,2-b]pyridazin-6-yl)-7H-pyrrolo [2,3-d]pyrimidin-2-amine [1090];
5-(3-(difluoromethyl)imidazo [1,2-b] pyridazin-6-yl)-N-((1-methylcyclopropyl)me thyl)-7H-pyrrolo [2,3-d]pyrimidin-2-amine [1091];
5-(3-(difluoromethyl)imidazo[1,2-b] pyridazin-6-yl)-N-4 1-(trifluoromethyl)cyclopropyl)methyl)-7H-pyrrolo [2,3-d]pyrimidin-2-amine [1092];
N-(2-cyclopropyl-2,2-difluoroethyl)-5-(3-(difluoromethyl)imidazo [1,2-b]pyridazin-6-yl)-7H-pyrrolo [2,3-d]pyrimidin-2-amine [1093];
N-(3,3-difluorocyclobutyl)-5-(3-(difluoromethyl)imidazo[1,2-b]pyridazin-6-yl)-7H-pyrrolo [2,3-d]pyrimidin-2-amine [1094];
5-(3-(difluoromethyl)imidazo [1,2-b] pyridazin-6-yl)-N-((1-fluorocyclobu tyl)methyl)-7H-pyrrolo [2,3-d]pyrimidin-2-amine [1095];
N-(4,4-difluorocyclohexyl)-5-(3-(difluoromethyl)imidazo [1,2-b] pyridazin-6-yl)-7H-pyrrolo [2,3-d]pyrimidin-2-amine [1096];
5-(3-(difluoromethyl)imidazo [1,2-b]pyridazin-6-yl)-N-(cis-4-methoxycyclohexyl)-7H-pyrrolo [2,3-d]pyrimidin-2-amine [1097];
5-(3-(difluoromethyl)imidazo[1,2-b] pyridazin-6-yl)-N-((1-fluorocyclohexyl)me thyl)-7H-pyrrolo [2,3-d]pyrimidin-2-amine [1098];
N-((4,4-difluorocyclohexyl)methyl)-5-(3-(difluoromethyl)imidazo [1,2-b]pyridazin-6-yl)-7H-pyrrolo [2,3-d]pyrimidin-2-amine [1099];
5-(3-(difluoromethyl)imidazo[1,2-b]pyridazin-6-yl)-N-(1-methylpiperidin-4-yl)-7H-pyrrolo [2,3-d]pyrimidin-2-amine [1100];
5-(3-(difluoromethyl)imidazo[1,2-b] pyridazin-6-yl)-N-((1-methylpiperidin-4-yl)me thyl)-7H-pyrrolo [2,3-d]pyrimidin-2-amine [1101];
5-(3-(difluoromethyl)imidazo[1,2-b]pyridazin-6-yl)-N-(tetrahydro-2H-pyran-4-yl)-7H-pyrrolo [2,3-d]pyrimidin-2-amine [1102];
5-(3-(difluoromethyl)imidazo[1,2-b]pyridazin-6-yl)-N-(3-(4-me thylpiperazin-1-yl)phenyl)-7H-pyrrolo [2,3-d]pyrimidin-2-amine [1103];
5-(3-(difluoromethyl)imidazo[1,2-b]pyridazin-6-yl)-N-(pyridin-4-yl)-7H-pyrrolo [2,3-d]pyrimidin-2-amine [1104];
5-(3-(difluoromethyl)imidazo[1,2-b]pyridazin-6-yl)-N-(2-(4-me thylpiperazin-1-yl)pyridin-4-yl)-7H-pyrrolo [2,3-d]pyrimidin-2-amine [1105];
5-(3-(difluoromethyl)imidazo[1,2-b]pyridazin-6-yl)-N-(6-(4-me thylpiperazin-1-yl)pyridin-3-yl)-7H-pyrrolo [2,3-d]pyrimidin-2-amine [1106];
5-(3-(difluoromethyl)imidazo[1,2-b]pyridazin-6-yl)-N-46-(4-methylpiperazin-1-yl)pyridin-3-yl)methyl)-7H-pyrrolo [2,3-d]pyrimidin-2-amine [1107];
6-(5-(3-(difluoromethyl)imidazo [1,2-b]pyridazin-6-yl)-7H-pyrrolo [2,3-d]pyrimidin-2-yl)quinoline [1108];
5-(3-(difluoromethyl)imidazo[1,2-b]pyridazin-6-yl)-4-(1-methyl-1H-pyrazol-4-yl)-7H-pyrrolo [2,3-d]pyrimidine [1109];
5-(3-(difluoromethyl)imidazo[1,2-b]pyridazin-6-yl)-N-(trans-4-morpho linocyclohexyl)-7H-pyrrolo [2,3-d]pyrimidin-4-amine [1110];
2-i sobutyl-5-(pyrazolo [1,5-a]pyridin-5-yl)-7H-pyrrolo [2,3-d]pyrimidine [1112];
2-i sobutyl-4-methoxy-5-(pyrazolo [1,5-a]pyridin-5-yl)-7H-pyrrolo [2,3-d] pyrimidine [1113];
5-(pyrazolo [1,5-a]pyridin-5-yl)-2-(3,3,3-trifluoropropyl)-7H-pyrrolo [2,3-d]pyrimidine [1114];
2-cyclopropyl-5-(pyrazolo [1,5-a] pyridin-5-yl)-7H-pyrrolo [2,3-d]pyrimidine [1115];
2-cyclobutyl-5-(pyrazolo [1,5-a]pyridin-5-yl)-7H-pyrrolo [2,3-d]pyrimidine [1116];
2-(cyclobutylmethyl)-5-(pyrazolo [1,5-a]pyridin-5-yl)-7H-pyrrolo [2,3-d] pyrimidine [1117];
5-(pyrazolo [1,5-a]pyridin-5-yl)-7H-pyrrolo [2,3-d]pyrimidin-2-amine [1118];
N-methyl-5-(pyrazolo [1,5-a]pyridin-5-yl)-7H-pyrrolo [2,3-d]pyrimidin-2-amine [1119];
N-ethyl-5-(pyrazolo [1,5-a] pyridin-5-yl)-7H-pyrrolo [2,3-d]pyrimidin-2-amine [1120];
N-isopropyl-5-(pyrazolo [1,5-a]pyridin-5-yl)-7H-pyrrolo [2,3-d]pyrimidin-2-amine [1121];
N-isobutyl-5-(pyrazolo [1,5-a] pyridin-5-yl)-7H-pyrrolo [2,3-d]pyrimidin-2-amine [1122];
N-neopentyl-5-(pyrazolo [1,5-a] pyridin-5-yl)-7H-pyrrolo [2,3-d]pyrimidin-2-amine [1123];
(S)-N-(sec-butyl)-5-(pyrazolo [1,5-a]pyridin-5-yl)-7H-pyrrolo [2,3-d]pyrimidin-2-amine [1124];
N-(2-fluoro-2-methylpropyl)-5-(pyrazolo [1,5-a]pyridin-5-yl)-7H-pyrrolo [2,3-d] pyrimidin-2-amine [1125];
N-(2,2-difluoropropyl)-5-(pyrazolo [1,5-a]pyridin-5-yl)-7H-pyrrolo [2,3-d]pyrimidin-2-amine [1126];
5-(pyrazolo [1,5-a]pyridin-5-yl)-N-(2,2,2-trifluoroethyl)-7H-pyrrolo [2,3-d]pyrimidin-2-amine [1127];
(R)-5-(pyrazolo [1,5-a]pyridin-5-yl)-N-(1, 1, 1-trifluoropropan-2-yl)-7H-pyrrolo [2,3-d]pyrimidin-2-amine [1128];
(S)-5-(pyrazolo [1,5-a]pyridin-5-yl)-N-(1, 1, 1-trifluoropropan-2-yl)-7H-pyrrolo [2,3-d]pyrimidin-2-amine [1129];
5-(pyrazolo [1,5-a]pyridin-5-yl)-N-(3 ,3 ,3-trifluoropropyl)-7H-pyrrolo [2,3-d]pyrimidin-2-amine [1130];
(S)-N-(1-methoxypropan-2-yl)-5-(pyrazolo [1,5-a]pyridin-5-yl)-7H-pyrrolo [2,3-d]pyrimidin-2-amine [1131];
2-ethoxy-5-(pyrazolo [1,5-a]pyridin-5-yl)-7H-pyrrolo [2,3-d]pyrimidine [1132];
N-((1-fluorocyclopropyl)methyl)-5-(pyrazolo [1,5-a] pyridin-5-yl)-7H-pyrrolo [2,3-d] pyrimidin-2-amine [1133];
(R)-N-(1-cyclopropylethyl)-5-(pyrazolo [1,5-a] pyridin-5-yl)-7H-pyrrolo [2,3-d]pyrimidin-2-amine [1134];
N-((1-methylcyclopropyl)methyl)-5-(pyrazolo [1,5-a] pyridin-5-yl)-7H-pyrrolo [2,3-d]pyrimidin-2-amine [1135];
5-(pyrazolo [1,5-a]pyridin-5-yl)-N-4 1-(trifluoromethyl)cyclopropyl)methyl)-7H-pyrrolo [2,3-d]pyrimidin-2-amine [1136];
N-(2-cyclopropyl-2,2-difluoroethyl)-5-(pyrazolo [1,5-a] pyridin-5-yl)-7H-pyrrolo [2,3-d]pyrimidin-2-amine [1137];
N-(3,3-difluorocyclobutyl)-5-(pyrazolo [1,5-a]pyridin-5-yl)-7H-pyrrolo [2,3-d] pyrimidin-2-amine [1138];
N-((1-fluorocyclobutypmethyl)-5-(pyrazolo [1,5-a]pyridin-5-yl)-7H-pyrrolo [2,3-d]pyrimidin-2-amine [1139];
N-(cis-3-methoxycyclobutyl)-5-(pyrazolo [1,5-a]pyridin-5-yl)-7H-pyrrolo [2,3-d]pyrimidin-2-amine [1140];
5-(pyrazolo [1,5-a]pyridin-5-yl)-N-(2-oxaspiro [3 . 3 ]heptan-6-yl)-7H-pyrrolo [2,3-d]pyrimidin-2-amine [1141];
N-(4,4-difluorocyclohexyl)-5-(pyrazolo [1,5-a]pyridin-5-yl)-7H-pyrrolo [2,3-d]pyrimidin-2-amine [1142];
N-(cis-4-methoxycyclohexyl)-5-(pyrazolo [1,5-a]pyridin-5-yl)-7H-pyrrolo [2,3-d]pyrimidin-2-amine [1143];
N-((1-fluorocyclohexyl)methyl)-5-(pyrazolo [1,5-a]pyridin-5-yl)-7H-pyrrolo [2,3-d]pyrimidin-2-amine [1144];
N-((4,4-difluorocyclohexyl)methyl)-5-(pyrazolo [1,5-a] pyridin-5-yl)-7H-pyrrolo [2,3-d]pyrimidin-2-amine [1145];
N-(1-methylpiperidin-4-yl)-5-(pyrazolo [1,5-a]pyridin-5-yl)-7H-pyrrolo [2,3-d]pyrimidin-2-amine [1146];
N-((1-methylpiperidin-4-yl)methyl)-5-(pyrazolo [1,5-a]pyridin-5-yl)-7H-pyrrolo [2,3-d] pyrimidin-2-amine [1147];
5-(pyrazolo [1,5-a]pyridin-5-yl)-N-(tetrahydro -2H-pyran-4-yl)-7H-pyrrolo [2,3-d]pyrimidin-2-amine [1148];
N-(3-(4-methylpiperazin-1-yl)phenyl)-5-(pyrazolo [1,5-a]pyridin-5-yl)-7H-pyrrolo [2,3-d]pyrimidin-2-amine [1149];
5-(pyrazolo [1,5-a]pyridin-5-yl)-N-(pyridin-4-yl)-7H-pyrrolo [2,3-d]pyrimidin-2-amine [1150];
N-(2-(4-methylpiperazin-1-yl)pyridin-4-yl)-5-(pyrazolo [1,5-a]pyridin-5-yl)-7H-pyrrolo [2,3-d]pyrimidin-2-amine [1151];
N-(6-(4-methylpiperazin-1-yl)pyridin-3-yl)-5-(pyrazolo [1,5-a]pyridin-5-yl)-7H-pyrrolo [2,3-d]pyrimidin-2-amine [1152];
N-((6-(4-methylpiperazin-1-yl)pyridin-3-yl)methyl)-5-(pyrazolo [1,5-a]pyridin-5-yl)-7H-pyrrolo [2,3-d]pyrimidin-2-amine [1153];
6-(5-(pyrazolo [1,5-a]pyridin-5-yl)-7H-pyrrolo [2,3-d]pyrimidin-2-yl)quinoline [1154];
4-(1-methyl-1H-pyrazol-4-yl)-5-(pyrazolo [1,5-a]pyridin-5-yl)-7H-pyrrolo [2,3-d] pyrimidine [1155];
N-(trans-4-morpholinocyclohexyl)-5-(pyrazolo [1,5-a]pyridin-5-yl)-7H-pyrrolo [2,3-d]pyrimidin-4-amine [1156];
N-(2-fluoro-2-methylpropyl)-5-(3-fluoropyrazolo [1,5-a]pyridin-5-yl)-7H-pyrrolo [2,3-d]pyrimidin-2-amine [1157];
2-i sobutyl-5-(3-(1-methyl-1H-pyrazol-4-yl)pyrazolo [1,5-a]pyridin-5-yl)-7H-pyrrolo [2,3-d]pyrimidine [1159];
5-(3-(1-methyl-1H-pyrazol-4-yl)pyrazolo [1,5-a]pyridin-5-yl)-2-(3,3,3-trifluoropropyl)-7H-pyrrolo [2,3-d]pyrimidine [1160];
2-cyclopropyl-5-(3-(1-methyl-1H-pyrazol-4-yl)pyrazolo [1,5-a]pyridin-5-yl)-7H-pyrrolo [2,3-d]pyrimidine [1161];
2-cyclobutyl-5-(3-(1-methyl-1H-pyrazol-4-yl)pyrazolo [1,5-a]pyridin-5-yl)-7H-pyrrolo [2,3-d]pyrimidine [1162];
2-(cyclobutylmethyl)-5-(3-(1-methyl-1H-pyrazol-4-yl)pyrazolo[1,5-a]pyridin-5-yl)-7H-pyrrolo[2,3-d]pyrimidine [1163];
5-(3-(1-methyl-1H-pyrazol-4-yl)pyrazolo[1,5-a]pyridin-5-yl)-7H-pyrrolo[2,3-d]pyrimidin-2-amine [1164];
N-methyl-5-(3-(1-methyl-1H-pyrazol-4-yl)pyrazolo[1,5-a]pyridin-5-yl)-7H-pyrrolo[2,3-d]pyrimidin-2-amine [1165];
N-ethyl-5-(3-(1-methyl-1H-pyrazol-4-yl)pyrazolo[1,5-a]pyridin-5-yl)-7H-pyrrolo[2,3-d]pyrimidin-2-amine [1166];
N-isopropyl-5-(3-(1-methyl-1H-pyrazol-4-yl)pyrazolo[1,5-a]pyridin-5-yl)-7H-pyrrolo[2,3-d]pyrimidin-2-amine [1167];
N-isobutyl-5-(3-(1-methyl-1H-pyrazol-4-yl)pyrazolo[1,5-a]pyridin-5-yl)-7H-pyrrolo[2,3-d]pyrimidin-2-amine [1168];
5-(3-(1-methyl-1H-pyrazol-4-yl)pyrazolo[1,5-a]pyridin-5-yl)-N-neopentyl-7H-pyrrolo[2,3-d]pyrimidin-2-amine [1169];
(S)-N-(sec-butyl)-5-(3-(1-methyl-1H-pyrazol-4-yl)pyrazolo[1,5-a]pyridin-5-yl)-7H-pyrrolo[2,3-d]pyrimidin-2-amine [1170];
N-(2-fluoro-2-methylpropyl)-5-(3-(1-methyl-1H-pyrazol-4-yl)pyrazolo[1,5-a]pyridin-5-yl)-7H-pyrrolo[2,3-d]pyrimidin-2-amine [1171];
N-(2,2-difluoropropyl)-5-(3-(1-methyl-1H-pyrazol-4-yl)pyrazolo[1,5-a]pyridin-5-yl)-7H-pyrrolo[2,3-d]pyrimidin-2-amine [1172];
5-(3-(1-methyl-1H-pyrazol-4-yl)pyrazolo[1,5-a]pyridin-5-yl)-N-(2,2,2-trifluoroethyl)-7H-pyrrolo[2,3-d]pyrimidin-2-amine [1173];
(R)-5-(3-(1-methyl-1H-pyrazol-4-yl)pyrazolo[1,5-a]pyridin-5-yl)-N-(1,1,1-trifluoropropan-2-yl)-7H-pyrrolo[2,3-d]pyrimidin-2-amine [1174];
(S)-5-(3-(1-methyl-1H-pyrazol-4-yl)pyrazolo[1,5-a]pyridin-5-yl)-N-(1,1,1-trifluoropropan-2-yl)-7H-pyrrolo[2,3-d]pyrimidin-2-amine [1175];
5-(3-(1-methyl-1H-pyrazol-4-yl)pyrazolo[1,5-a]pyridin-5-yl)-N-(3,3,3-trifluoropropyl)-7H-pyrrolo[2,3-d]pyrimidin-2-amine [1176];
(S)-N-(1-methoxypropan-2-yl)-5-(3-(1-methyl-1H-pyrazol-4-yl)pyrazolo[1,5-a]pyridin-5-yl)-7H-pyrrolo[2,3-d]pyrimidin-2-amine [1177];
2-ethoxy-5-(3-(1-methyl-1H-pyrazol-4-yl)pyrazolo[1,5-a]pyridin-5-yl)-7H-pyrrolo[2,3-d]pyrimidine [1178];
N-((1-fluorocyclopropyl)methyl)-5-(3-(1-methyl-1H-pyrazol-4-yl)pyrazolo[1,5-a]pyridin-5-yl)-7H-pyrrolo[2,3-d]pyrimidin-2-amine [1179];
(R)-N-(1-cyclopropylethyl)-5-(3-(1-methyl-1H-pyrazol-4-yl)pyrazolo[1,5-a]pyridin-5-yl)-7H-pyrrolo[2,3-d]pyrimidin-2-amine [1180];
5-(3-(1-methyl-1H-pyrazol-4-yl)pyrazolo[1,5-a]pyridin-5-yl)-N-((1-methylcyclopropyl)methyl)-7H-pyrrolo[2,3-d]pyrimidin-2-amine [1181];
5-(3-(1-methyl-1H-pyrazol-4-yl)pyrazolo[1,5-a]pyridin-5-yl)-N-41-(trifluoromethyl)cyclopropyl)methyl)-7H-pyrrolo[2,3-d]pyrimidin-2-amine [1182];
N-(2-cyclopropyl-2,2-difluoroethyl)-5-(3-(1-methyl-1H-pyrazol-4-yl)pyrazolo[1,5-a]pyridin-5-yl)-7H-pyrrolo[2,3-d]pyrimidin-2-amine [1183];
N-(3,3-difluorocyclobutyl)-5-(3-(1-methyl-1H-pyrazol-4-yl)pyrazolo[1,5-a]pyridin-5-yl)-7H-pyrrolo[2,3-d]pyrimidin-2-amine [1184];
N-((1-fluorocyclobutyl)methyl)-5-(3-(1-methyl-1H-pyrazol-4-yl)pyrazolo[1,5-a]pyridin-5-yl)-7H-pyrrolo[2,3-d]pyrimidin-2-amine [1185];
N-(4,4-difluorocyclohexyl)-5-(3-(1-methyl-1H-pyrazol-4-yl)pyrazolo[1,5-a]pyridin-5-yl)-7H-pyrrolo[2,3-d]pyrimidin-2-amine [1186];
N-(cis-4-methoxycyclohexyl)-5-(3-(1-methyl-1H-pyrazol-4-yl)pyrazolo[1,5-a]pyridin-5-yl)-7H-pyrrolo[2,3-d]pyrimidin-2-amine [1187];
N-((1-fluorocyclohexyl)methyl)-5-(3-(1-methyl-1H-pyrazol-4-yl)pyrazolo[1,5-a]pyridin-5-yl)-7H-pyrrolo[2,3-d]pyrimidin-2-amine [1188];
N-((4,4-difluorocyclohexyl)methyl)-5-(3-(1-methyl-1H-pyrazol-4-yl)pyrazolo[1,5-a]pyridin-5-yl)-7H-pyrrolo[2,3-d]pyrimidin-2-amine [1189];
5-(3-(1-methyl-1H-pyrazol-4-yl)pyrazolo[1,5-a]pyridin-5-yl)-N-(1-methylpiperidin-4-yl)-7H-pyrrolo[2,3-d]pyrimidin-2-amine [1190];
5-(3-(1-methyl-1H-pyrazol-4-yl)pyrazolo[1,5-a]pyridin-5-yl)-N-((1-methylpiperidin-4-yl)methyl)-7H-pyrrolo[2,3-d]pyrimidin-2-amine [1191];
5-(3-(1-methyl-1H-pyrazol-4-yl)pyrazolo[1,5-a]pyridin-5-yl)-N-(tetrahydro-2H-pyran-4-yl)-7H-pyrrolo[2,3-d]pyrimidin-2-amine [1192];
5-(3-(1-methyl-1H-pyrazol-4-yl)pyrazolo[1,5-a]pyridin-5-yl)-N-(3-(4-methylpiperazin-1-yl)phenyl)-7H-pyrrolo[2,3-d]pyrimidin-2-amine [1193];
5-(3-(1-methyl-1H-pyrazol-4-yl)pyrazolo[1,5-a]pyridin-5-yl)-N-(pyridin-4-yl)-7H-pyrrolo[2,3-d]pyrimidin-2-amine [1194];
5-(3-(1-methyl-1H-pyrazol-4-yl)pyrazolo[1,5-a]pyridin-5-yl)-N-(2-(4-methylpiperazin-1-yl)pyridin-4-yl)-7H-pyrrolo[2,3-d]pyrimidin-2-amine [1195];
5-(3-(1-methyl-1H-pyrazol-4-yl)pyrazolo[1,5-a]pyridin-5-yl)-N-(6-(4-methylpiperazin-1-yl)pyridin-3-yl)-7H-pyrrolo[2,3-d]pyrimidin-2-amine [1196];
5-(3-(1-methyl-1H-pyrazol-4-yl)pyrazolo [1,5-a]pyridin-5-yl)-N-46-(4-methylpiperazin-1-yl)pyridin-3-yl)methyl)-7H-pyrrolo [2,3-d] pyrimidin-2-amine [1197];
6-(5-(3-(1-methyl-1H-pyrazol-4-yl)pyrazolo [1,5-a]pyridin-5-yl)-7H-pyrrolo [2,3-d]pyrimidin-2-yl)quinoline [1198];
4-(1-methyl-1H-pyrazol-4-yl)-5-(3-(1-methyl-1H-pyrazol-4-yl)pyrazolo [1,5-a]pyridin-5-yl)-7H-pyrrolo [2,3-d]pyrimidine [1199];
5-(3-(1-methyl-1H-pyrazol-4-yl)pyrazolo [1,5-a]pyridin-5-yl)-N-(trans-4-morpholinocyclohexyl)-7H-pyrrolo [2,3-d]pyrimidin-4-amine [1200];
(5-(2-isobutyl-7H-pyrrolo [2,3-d]pyrimidin-5-yl)pyrazolo [1,5-a]pyridin-3-yl)(piperidin-1-yl)methanone [1202];
piperidin-1-yl(5-(2-(3,3,3-trifluoropropyl)-7H-pyrrolo [2,3-d] pyrimidin-5-yl)pyrazolo [1,5-a] pyridin-3-yl)methanone [1203];
(5-(2-cyclopropyl-7H-pyrrolo [2,3-d] pyrimidin-5-yl)pyrazolo [1,5-a]pyridin-3-yl)(piperidin-1-yl)methanone [1204];
(5-(2-cyclobutyl-7H-pyrrolo [2,3-d]pyrimidin-5-yl)pyrazolo [1,5-a]pyridin-3-yl)(piperidin-1-yl)methanone [1205];
(5-(2-(cyclobutylmethyl)-7H-pyrrolo [2,3-d] pyrimidin-5-yl)pyrazolo [1,5-a] pyridin-3-yl)(piperidin-1-yl)methanone [1206];
(5-(2-amino-7H-pyrrolo [2,3-d] pyrimidin-5-yl)pyrazolo [1,5-a]pyridin-3-yl)(piperidin-1-yl)methanone [1207];
(5-(2-(methylamino)-7H-pyrrolo [2,3-d] pyrimidin-5-yl)pyrazolo [1,5-a]pyridin-3-yl)(piperidin-1-yl)methanone [1208];
(5-(2-(ethylamino)-7H-pyrrolo [2,3-d]pyrimidin-5-yl)pyrazolo [1,5-a]pyridin-3-yl)(piperidin-1-yl)methanone [1209];
(5-(2-(isopropylamino)-7H-pyrrolo [2,3-d] pyrimidin-5-yl)pyrazolo [1,5-a] pyridin-3-yl)(pipe ridin-1-yl)methanone [1210];
(5-(2-(isobutylamino)-7H-pyrrolo [2,3-d]pyrimidin-5-yl)pyrazolo [1,5-a]pyridin-3-yl)(piperidin-1-yl)methanone [1211];
(5-(2-(neopentylamino)-7H-pyrrolo [2,3-d]pyrimidin-5-yl)pyrazolo [1,5-a]pyridin-3-yl)(piperidin-1-yl)methanone [1212];
(S)-(5-(2-(sec-butylamino)-7H-pyrrolo [2,3-d] pyrimidin-5-yl)pyrazolo [1,5-a]pyridin-3-yl)(piperidin-1-yl)methanone [1213];
(5-(2-((2-fluoro-2-methylpropyl)amino)-7H-pyrrolo [2,3-d]pyrimidin-5-yl)pyrazolo [1,5-a]pyridin-3-yl)(piperidin-1-yl)methanone [1214];
(5-(2-((2,2-difluoropropyl)amino)-7H-pyrrolo [2,3-d] pyrimidin-5-yl)pyrazolo [1,5-a]pyridin-3-yl)(piperidin-1-yl)methanone [1215];
piperidin-1-yl(5-(2-((2,2,2-trifluoroethyDamino)-7H-pyrrolo [2,3-d] pyrimidin-5-yl)pyrazolo [1,5-a] pyridin-3-yl)methanone [1216];
(R)-piperidin-1-yl(5-(2-((1,1,1-trifluoropropan-2-yl)amino)-7H-pyrrolo [2,3-d]pyrimidin-5-yl)pyrazolo [1,5-a]pyridin-3-yl)methanone [1217];
(S)-piperidin-1-yl(5-(2-((1,1,1-trifluoropropan-2-yl)amino)-7H-pyrrolo [2,3-d] pyrimidin-5-yl)pyrazolo [1,5-a]pyridin-3-yl)methanone [1218];
piperidin-1-yl(5-(2-((3,3,3-trifluoropropyl)amino)-7H-pyrrolo [2,3-d] pyrimidin-5-yl)pyrazolo [1,5-a] pyridin-3-yl)methanone [1219];
(S)-(5-(2-((l-methoxypropan-2-yl)amino)-7H-pyrrolo [2,3-d] pyrimidin-5-yl)pyrazolo [1,5-a] pyridin-3-yl)(pipe ridin-1-yl)methanone [1220];
(5-(2-ethoxy-7H-pyrrolo [2,3-d]pyrimidin-5-yl)pyrazolo [1,5-a]pyridin-3-yl)(piperidin-1-yl)methanone [1221];
(5-(2-(((1-fluorocyclopropyl)methyl)amino)-7H-pyrrolo [2,3-d] pyrimidin-5-yl)pyrazolo [1,5-a] pyridin-3-yl)(pipe ridin-1-yl)methanone [1222];
(R)-(5-(2-((l-cyclopropylethyl)amino)-7H-pyrrolo [2,3-d]pyrimidin-5-yl)pyrazolo [1,5-a]pyridin-3-yl)(piperidin-1-yl)methanone [1223];
(5-(2-(((1-methylcyclopropyl)methyl)amino)-7H-pyrrolo [2,3-d]pyrimidin-5-yl)pyrazolo [1,5-a] pyridin-3-yl)(pipe ridin-1-yl)methanone [1224];
piperidin-1-yl(5-(2-(41-(trifluoromethyl)cyclopropyl)methyl)amino)-7H-pyrrolo [2,3-d]pyrimidin-5-yl)pyrazolo [1,5-a]pyridin-3-yl)methanone [1225];
(5-(2-((2-cyclopropyl-2,2-difluoroethyl)amino)-7H-pyrrolo [2,3-d]pyrimidin-5-yl)pyrazolo [1,5-a] pyridin-3-yl)(pipe ridin-1-yl)methanone [1226];
(5-(2-((3,3-difluorocyclobutyl)amino)-7H-pyrrolo [2,3-d] pyrimidin-5-yl)pyrazolo [1,5-a] pyridin-3-yl)(piperidin-1-yl)methanone [1227];
(5-(2-(((1-fluorocyclobutyl)methyl)amino)-7H-pyrrolo [2,3-d]pyrimidin-5-yl)pyrazolo [1,5-a] pyridin-3-yl)(pipe ridin-1-yl)methanone [1228];
(5-(2-((4,4-difluorocyclohexyl)amino)-7H-pyrrolo [2,3-d]pyrimidin-5-yl)pyrazolo [1,5-a]pyridin-3-yl)(piperidin-1-yl)methanone [1229];
(5-(2-((cis-4-methoxycyclohexyl)amino)-7H-pyrrolo [2,3-d] pyrimidin-5-yl)pyrazolo [1,5-a] pyridin-3-yl)(pipe ridin-1-yl)methanone [1230];
(5-(2-(((1-fluorocyclohexyl)methyl)amino)-7H-pyrrolo [2,3-d] pyrimidin-5-yl)pyrazolo [1,5-a] pyridin-3-yl)(pipe ridin-1-yl)methanone [1231];
(5-(2-(((4,4-difluorocyclohexyl)methyl)amino)-7H-pyrrolo [2,3-d] pyrimidin-5-yl)pyrazolo [1,5-a] pyridin-3-yl)(pipe ridin-1-yl)methanone [1232];
(5-(2-((l-methylpiperidin-4-yl)amino)-7H-pyrrolo [2,3-d] pyrimidin-5-yl)pyrazolo [1,5-a]pyridin-3-yl)(piperidin-1-yl)methanone [1233];
(5-(2-(((l-methylpiperidin-4-yl)methyl)amino)-7H-pyrrolo [2,3-d] pyrimidin-5-yl)pyrazolo [1,5-a] pyridin-3-yl)(pipe ridin-1-yl)methanone [1234];
piperidin-1-yl(5-(2-((tetrahydro-2H-pyran-4-yl)amino)-7H-pyrrolo [2,3-d] pyrimidin-5-yl)pyrazolo [1,5-a]pyridin-3-yl)methanone [1235];
(5-(2-((3-(4-methylpiperazin-1-yl)phenyl)amino)-7H-pyrrolo [2,3-d] pyrimidin-5-yl)pyrazolo [1,5-a] pyridin-3-yl)(pipe ridin-1-yl)methanone [1236];
piperidin-1-yl(5-(2-(pyridin-4-ylamino)-7H-pyrrolo [2,3-d] pyrimidin-5-yl)pyrazolo [1,5-a] pyridin-3-yl)methanone [1237];
(5-(2-((2-(4-methylpiperazin-1-yl)pyridin-4-yl)amino)-7H-pyrrolo [2,3-d] pyrimidin-5-yl)pyrazolo [1,5-a]pyridin-3-yl)(piperidin-1-yl)methanone [1238];
(5-(2-((6-(4-methylpiperazin-1-yl)pyridin-3-yl)amino)-7H-pyrrolo [2,3-d] pyrimidin-5-yl)pyrazolo [1,5-a]pyridin-3-yl)(piperidin-1-yl)methanone [1239];
(5-(2-(((6-(4-methylpiperazin-1-yl)pyridin-3-yl)methyl)amino)-7H-pyrrolo [2,3-d]pyrimidin-5-yl)pyrazolo [1,5-a]pyridin-3-yl)(piperidin-1-yl)methanone [1240];
piperidin-1-yl(5-(2-(quinolin-6-yl)-7H-pyrrolo [2,3-d]pyrimidin-5-yl)pyrazolo [1,5-a]pyridin-3-yl)methanone [1241];
(5-(4-(1-methyl-1H-pyrazol-4-yl)-7H-pyrrolo [2,3-d] pyrimidin-5-yl)pyrazolo [1,5-a] pyridin-3-yl)(piperidin-1-yl)methanone [1242];
(5-(4-((trans-4-morpholinocyclohexyl)amino)-7H-pyrrolo [2,3-d] pyrimidin-5-yl)pyrazolo [1,5-a] pyridin-3-yl)(pipe ridin-1-yl)methanone [1243];
(5-(4-((cyclopropylmethyl)amino)-7H-pyrrolo [2,3-d]pyrimidin-5-yl)pyrazolo [1,5-a]pyridin-3-yl)(4-methylpiperazin-1-yl)methanone [1244];
5-(2-cyclopropyl-7H-pyrrolo [2,3-d]pyrimidin-5-yl)-N,N-dimethylpyrazolo [1,5-a]pyridine-3-carboxamide [1245];
N-(2,2-difluoroethyl)-5-(24 sobutyl-7H-pyrrolo [2,3-d]pyrimidin-5-yl)pyrazolo [1,5-a]pyridine-3-carboxamide [1247];
N-(2,2-difluoroethyl)-5-(2-(3,3,3-trifluoropropyl)-7H-pyrrolo [2,3-d] pyrimidin-5-yl)pyrazolo [1,5-a] pyridine-3-carboxamide [1248];
5-(2-cyclopropyl-7H-pyrrolo [2,3-d]pyrimidin-5-yl)-N-(2,2-difluoroethyppyrazolo [1,5-a] pyridine-3-carboxamide [1249];
5-(2-cyclobutyl-7H-pyrrolo [2,3-d] pyrimidin-5-yl)-N-(2,2-difluoroethyl)pyrazolo [1,5-a] pyridine-3-carboxamide [1250];
5-(2-(cyclobutylmethyl)-7H-pyrrolo [2,3-d]pyrimidin-5-yl)-N-(2,2-difluoroethyppyrazolo [1,5-a] pyridine-3-carboxamide [1251];
5-(2-amino-7H-pyrrolo [2,3-d] pyrimidin-5-yl)-N-(2,2-difluoroethyl)pyrazolo [1,5-a]pyridine-3-carboxamide [1252];
N-(2,2-difluoroethyl)-5-(2-(methylamino)-7H-pyrrolo [2,3-d] pyrimidin-5-yl)pyrazolo [1,5-a] pyridine-3-carboxamide [1253];
N-(2,2-difluoroethyl)-5-(2-(ethylamino)-7H-pyrrolo [2,3-d] pyrimidin-5-yl)pyrazolo [1,5-a] pyridine-3-carboxamide [1254];
N-(2,2-difluoroethyl)-5-(2-(i sop ropylamino)-7H-pyrrolo [2,3-d]pyrimidin-5-yl)pyrazolo [1,5-a] pyridine-3-carboxamide [1255];
N-(2,2-difluoroethyl)-5-(2-(isobutylamino)-7H-pyrrolo [2,3-d]pyrimidin-5-yl)pyrazolo [1,5-a] pyridine-3-carboxamide [1256];
N-(2,2-difluoroethyl)-5-(2-(neopentylamino)-7H-pyrrolo [2,3-d]pyrimidin-5-yl)pyrazolo [1,5-a] pyridine-3-carboxamide [1257];
(S)-5-(2-(sec-butylamino)-7H-pyrrolo [2,3-d] pyrimidin-5-yl)-N-(2,2-difluoroethyl)pyrazolo [1,5-a] pyridine-3-carboxamide [1258];
N-(2,2-difluoroethyl)-5-(2-((2-fluoro-2-methylpropyl)amino)-7H-pyrrolo [2,3-d]pyrimidin-5-yl)pyrazolo [1,5-a]pyridine-3-carboxamide [1259];
N-(2,2-difluoroethyl)-5-(2-((2,2-difluoropropyl)amino)-7H-pyrrolo [2,3-d] pyrimidin-5-yl)pyrazolo [1,5-a]pyridine-3-carboxamide [1260];
N-(2,2-difluoroethyl)-5-(2-((2,2,2-trifluoroethyl)amino)-7H-pyrrolo [2,3-d] pyrimidin-5-yl)pyrazolo [1,5-a]pyridine-3-carboxamide [1261];
(R)-N-(2,2-difluoroethyl)-5-(2-((1,1,1-trifluoropropan-2-yl)amino)-7H-pyrrolo [2,3-d]pyrimidin-5-yl)pyrazolo[1,5-a]pyridine-3-carboxamide [1262];
(S)-N-(2,2-difluoroethyl)-5-(2-((1,1,1-trifluoropropan-2-yl)amino)-7H-pyrrolo [2,3-d]pyrimidin-5-yl)pyrazolo [1,5-a]pyridine-3-carboxamide [1263];
N-(2,2-difluoroethyl)-5-(2-((3,3,3-trifluoropropyl)amino)-7H-pyrrolo [2,3-d] pyrimidin-5-yl)pyrazolo [1,5-a]pyridine-3-carboxamide [1264];
(S)-N-(2,2-difluoroethyl)-5-(2-((l-methoxypropan-2-yl)amino)-7H-pyrrolo [2,3-d]pyrimidin-5-yl)pyrazolo [1,5-a]pyridine-3-carboxamide [1265];
N-(2,2-difluoroethyl)-5-(2-ethoxy-7H-pyrrolo [2,3-d]pyrimidin-5-yl)pyrazolo [1,5-a]pyridine-3-carboxamide [1266];
N-(2,2-difluoroethyl)-5-(2-(((1-fluorocyclopropyl)methyl)amino)-7H-pyrrolo [2,3-d]pyrimidin-5-yl)pyrazolo [1,5-a]pyridine-3-carboxamide [1267];
(R)-5-(2-((l-cyclopropylethyl)amino)-7H-pyrrolo [2,3-d]pyrimidin-5-yl)-N-(2,2-difluoroethyppyrazolo [1,5-a]pyridine-3-carboxamide [1268];
N-(2,2-difluoroethyl)-5-(2-(((l-methylcyclopropyl)methyl)amino)-7H-pyrrolo [2,3-d]pyrimidin-5-yl)pyrazolo [1,5-a]pyridine-3-carboxamide [1269];
N-(2,2-difluoroethyl)-5-(2-(((1-(trifluoromethyl)cyclopropyl)methyl)amino)-7H-pyrrolo [2,3-d]pyrimidin-5-yl)pyrazolo [1,5-a]pyridine-3-carboxamide [1270];
5-(2-((2-cyclopropyl-2,2-difluoroethyl)amino)-7H-pyrrolo [2,3-d]pyrimidin-5-yl)-N-(2,2-difluoroethyppyrazolo [1,5-a]pyridine-3-carboxamide [1271];
5-(2-((3,3-difluorocyclobutyl)amino)-7H-pyrrolo [2,3-d]pyrimidin-5-yl)-N-(2,2-difluoroethyppyrazolo [1,5-a]pyridine-3-carboxamide [1272];
N-(2,2-difluoroethyl)-5-(2-(((1-fluorocyclobutyl)methyl)amino)-7H-pyrrolo [2,3-d]pyrimidin-5-yl)pyrazolo [1,5-a]pyridine-3-carboxamide [1273];
5-(2-((4,4-difluorocyclohexyl)amino)-7H-pyrrolo [2,3-d]pyrimidin-5-yl)-N-(2,2-difluoroethyppyrazolo [1,5-a]pyridine-3-carboxamide [1274];
N-(2,2-difluoroethyl)-5-(2-((cis-4-methoxycyclohexyl)amino)-7H-pyrrolo [2,3-d]pyrimidin-5-yl)pyrazolo [1,5-a]pyridine-3-carboxamide [1275];
N-(2,2-difluoroethyl)-5-(2-(((1-fluorocyclohexyl)methyl)amino)-7H-pyrrolo [2,3-d]pyrimidin-5-yl)pyrazolo [1,5-a]pyridine-3-carboxamide [1276];
5-(2-(((4,4-difluorocyclohexyl)methyl)amino)-7H-pyrrolo [2,3-d] pyrimidin-5-yl)-N-(2,2-difluoroethyppyrazolo [1,5-a]pyridine-3-carboxamide [1277];
N-(2,2-difluoroethyl)-5-(2-((l-methylpiperidin-4-yl)amino)-7H-pyrrolo [2,3-d]pyrimidin-5-yl)pyrazolo [1,5-a]pyridine-3-carboxamide [1278];
N-(2,2-difluoroethyl)-5-(2-(((l-methylpiperidin-4-yl)methyl)amino)-7H-pyrrolo [2,3-d]pyrimidin-5-yl)pyrazolo[1,5-a]pyridine-3-carboxamide [1279];
N-(2,2-difluoroethyl)-5-(2-((tetrahydro-2H-pyran-4-yl)amino)-7H-pyrrolo [2,3-d] pyrimidin-5-yl)pyrazolo [1,5-a]pyridine-3-carboxamide [1280];
N-(2,2-difluoroethyl)-5-(2-((3-(4-methylpiperazin-1-yl)phenyl)amino)-7H-pyrrolo [2,3-d]pyrimidin-5-yl)pyrazolo [1,5-a]pyridine-3-carboxamide [1281];
N-(2,2-difluoroethyl)-5-(2-(pyridin-4-ylamino)-7H-pyrrolo [2,3-d]pyrimidin-5-yl)pyrazolo [1,5-a] pyridine-3-carboxamide [1282];
N-(2,2-difluoroethyl)-5-(2-42-(4-methylpiperazin-1-yl)pyridin-4-yl)amino)-7H-pyrrolo [2,3-d]pyrimidin-5-yl)pyrazolo [1,5-a]pyridine-3-carboxamide [1283];
2,2-difluoroethyl)-5-(2-46-(4-methylpiperazin-1-yl)pyridin-3-yl)amino)-7H-pyrrolo [2,3-d]pyrimidin-5-yl)pyrazolo [1,5-a]pyridine-3-carboxamide [1284];
N-(2,2-difluoroethyl)-5-(2-(46-(4-methylpiperazin-1-yl)pyridin-3-yl)methyl)amino)-7H-pyrrolo [2,3-d]pyrimidin-5-yl)pyrazolo [1,5-a]pyridine-3-carboxamide [1285];
N-(2,2-difluoroethyl)-5-(2-(quinolin-6-yl)-7H-pyrrolo [2,3-d] pyrimidin-5-yl)pyrazolo [1,5-a] pyridine-3-carboxamide [1286];
N-(2,2-difluoroethyl)-5-(4-(1-methyl-1H-pyrazol-4-yl)-7H-pyrrolo [2,3-d] pyrimidin-5-yl)pyrazolo [1,5-a]pyridine-3-carboxamide [1287];
N-(2,2-difluoroethyl)-5-(4-((trans-4-morpholinocyclohexyl)amino)-7H-pyrrolo [2,3-d]pyrimidin-5-yl)pyrazolo[1,5-a]pyridine-3-carboxamide [1288];
N-(2-fluoro-2-methylpropyl)-5-(2-isobutyl-7H-pyrrolo [2,3-d] pyrimidin-5-yl)pyrazolo [1,5-a] pyridine-3-carboxamide [1290];
N-(2-fluoro-2-methylpropyl)-5-(2-(3,3,3-trifluoropropyl)-7H-pyrrolo [2,3-d] pyrimidin-5-yl)pyrazolo [1,5-a]pyridine-3-carboxamide [1291];
5-(2-cyclopropyl-7H-pyrrolo [2,3-d]pyrimidin-5-yl)-N-(2-fluoro-2-methylpropyl)pyrazolo [1,5-a] pyridine-3-carboxamide [1292];
5-(2-cyclobutyl-7H-pyrrolo [2,3-d]pyrimidin-5-yl)-N-(2-fluoro-2-methylpropyl)pyrazolo [1,5-a] pyridine-3-carboxamide [1293];
5-(2-(cyclobutylmethyl)-7H-pyrrolo [2,3-d]pyrimidin-5-yl)-N-(2-fluoro-2-methylpropyl)pyrazolo [1,5-a]pyridine-3-carboxamide [1294];
5-(2-amino-7H-pyrrolo [2,3-d] pyrimidin-5-yl)-N-(2-fluoro-2-methylpropyl)pyrazolo [1,5-a] pyridine-3-carboxamide [1295];
N-(2-fluoro-2-methylpropyl)-5-(2-(methylamino)-7H-pyrrolo [2,3-d] pyrimidin-5-yl)pyrazolo [1,5-a] pyridine-3-carboxamide [1296];
5-(2-(ethylamino)-7H-pyrrolo [2,3-d]pyrimidin-5-yl)-N-(2-fluoro-2-methylpropyl)pyrazolo [1,5-a] pyridine-3-carboxamide [1297];
N-(2-fluoro-2-methylpropyl)-5-(2-(isopropylamino)-7H-pyrrolo [2,3-d]pyrimidin-5-yl)pyrazolo [1,5-a]pyridine-3-carboxamide [1298];
N-(2-fluoro-2-methylpropyl)-5-(2-(isobutylamino)-7H-pyrrolo [2,3-d] pyrimidin-5-yl)pyrazolo [1,5-a]pyridine-3-carboxamide [1299];
N-(2-fluoro-2-methylpropyl)-5-(2-(neopentylamino)-7H-pyrrolo [2,3-d]pyrimidin-5-yl)pyrazolo [1,5-a]pyridine-3-carboxamide [1300];
(S)-5-(2-(sec-butylamino)-7H-pyrrolo [2,3-d] pyrimidin-5-yl)-N-(2-fluoro-2-methylpropyl)pyrazolo [1,5-a]pyridine-3-carboxamide [1301];
N-(2-fluoro-2-methylpropyl)-5-(2-((2-fluoro-2-methylpropyl)amino)-7H-pyrrolo [2,3-d]pyrimidin-5-yl)pyrazolo [1,5-a]pyridine-3-carboxamide [1302];
5-(2-((2,2-difluoropropyl)amino)-7H-pyrrolo [2,3-d] pyrimidin-5-yl)-N-(2-fluoro-2-methylpropyl)pyrazolo [1,5-a]pyridine-3-carboxamide [1303];
N-(2-fluoro-2-methylpropyl)-5-(2-((2,2,2-trifluoroethyl)amino)-7H-pyrrolo [2,3-d]pyrimidin-5-yl)pyrazolo [1,5-a]pyridine-3-carboxamide [1304];
(R)-N-(2-fluoro-2-methylpropyl)-5-(2-((1,1,1-trifluoropropan-2-yl)amino)-7H-pyrrolo [2,3-d] pyrimidin-5-yl)pyrazolo [1,5-a]pyridine-3-carboxamide [1305];
(S)-N-(2-fluoro-2-methylpropyl)-5-(2-((1,1,1-trifluoropropan-2-yl)amino)-7H-pyrrolo [2,3-d] pyrimidin-5-yl)pyrazolo [1,5-a]pyridine-3-carboxamide [1306];
N-(2-fluoro-2-methylpropyl)-5-(2-((3,3,3-trifluoropropyl)amino)-7H-pyrrolo [2,3-d]pyrimidin-5-yl)pyrazolo [1,5-a]pyridine-3-carboxamide [1307];
(S)-N-(2-fluoro-2-methylpropyl)-5-(2-((l-methoxypropan-2-yl)amino)-7H-pyrrolo [2,3-d]pyrimidin-5-yl)pyrazolo [1,5-a]pyridine-3-carboxamide [1308];
5-(2-ethoxy-7H-pyrrolo [2,3-d] pyrimidin-5-yl)-N-(2-fluoro-2-methylpropyl)pyrazolo [1,5-a] pyridine-3-carboxamide [1309];
N-(2-fluoro-2-methylpropyl)-5-(2-(((1-fluorocyclopropyl)methyl)amino)-7H-pyrrolo [2,3-d]pyrimidin-5-yl)pyrazolo [1,5-a]pyridine-3-carboxamide [1310];
(R)-5-(2-((l-cyclopropylethyl)amino)-7H-pyrrolo [2,3-d]pyrimidin-5-yl)-N-(2-fluoro-2-methylpropyl)pyrazolo [1,5-a]pyridine-3-carboxamide [1311];
N-(2-fluoro-2-methylpropyl)-5-(2-(((l-methylcyclopropyl)methyl)amino)-7H-pyrrolo [2,3-d] pyrimidin-5-yl)pyrazolo [1,5-a]pyridine-3-carboxamide [1312];
N-(2-fluoro-2-methylpropyl)-5-(2-(((1-(trifluoromethyl)cyclopropyl)methyl)amino)-7H-pyrrolo [2,3-d]pyrimidin-5-yl)pyrazolo [1,5-a]pyridine-3-carboxamide [1313];
5-(2-((2-cyclopropyl-2,2-difluoroethyl)amino)-7H-pyrrolo [2,3-d]pyrimidin-5-yl)-N-(2-fluoro-2-methylpropyl)pyrazolo [1,5-a]pyridine-3-carboxamide [1314];
5-(2-((3,3-difluorocyclobutyl)amino)-7H-pyrrolo [2,3-d]pyrimidin-5-yl)-N-(2-fluoro-2-methylpropyl)pyrazolo [1,5-a]pyridine-3-carboxamide [1315];
N-(2-fluoro-2-methylpropyl)-5-(2-(((1-fluorocyclobutyl)methyl)amino)-7H-pyrrolo [2,3-d]pyrimidin-5-yl)pyrazolo [1,5-a]pyridine-3-carboxamide [1316];
5-(2-((4,4-difluorocyclohexyl)amino)-7H-pyrrolo [2,3-d]pyrimidin-5-yl)-N-(2-fluoro-2-methylpropyl)pyrazolo [1,5-a]pyridine-3-carboxamide [1317];
N-(2-fluoro-2-methylpropyl)-5-(2-((cis-4-methoxycyclohexyl)amino)-7H-pyrrolo [2,3-d]pyrimidin-5-yl)pyrazolo [1,5-a]pyridine-3-carboxamide [1318];
N-(2-fluoro-2-methylpropyl)-5-(2-(((1-fluorocyclohexyl)methyl)amino)-7H-pyrrolo [2,3-d]pyrimidin-5-yl)pyrazolo [1,5-a]pyridine-3-carboxamide [1319];
5-(2-4(4,4-difluorocyclohexyl)me thyl)amino)-7H-pyrrolo [2,3-d]pyrimidin-5-yl)-N-(2-fluoro-2-methylpropyl)pyrazolo [1,5-a]pyridine-3-carboxamide [1320];
N-(2-fluoro-2-methylpropyl)-5-(2-((l-methylpiperidin-4-yl)amino)-7H-pyrrolo [2,3-d]pyrimidin-5-yl)pyrazolo[1,5-a]pyridine-3-carboxamide [1321];
N-(2-fluoro-2-methylpropyl)-5-(2-(((l-methylpiperidin-4-yl)me thyl)amino)-7H-pyrrolo [2,3-d]pyrimidin-5-yl)pyrazolo [1,5-a]pyridine-3-carboxamide [1322];
N-(2-fluoro-2-methylpropyl)-5-(2-((tetrahydro -2H-pyran-4-yl)amino)-7H-pyrrolo [2,3-d] pyrimidin-5-yl)pyrazolo [1,5-a]pyridine-3-carboxamide [1323];
N-(2-fluoro-2-methylpropyl)-5-(2-((3-(4-methylpipe razin-1-yl)phenyl)amino)-7H-pyrrolo [2,3-d]pyrimidin-5-yl)pyrazolo [1,5-a]pyridine-3-carboxamide [1324];
N-(2-fluoro-2-methylpropyl)-5-(2-(pyridin-4-ylamino)-7H-pyrrolo [2,3-d]pyrimidin-5-yl)pyrazolo [1,5-a]pyridine-3-carboxamide [1325];
N-(2-fluoro-2-methylpropyl)-5-(2-((2-(4-methylpipe razin-1-yl)pyridin-4-yl)amino)-7H-pyrrolo [2,3-d]pyrimidin-5-yl)pyrazolo [1,5-a]pyridine-3-carboxamide [1326];
N-(2-fluoro-2-methylpropyl)-5-(2-((6-(4-methylpipe razin-1-yl)pyridin-3-yl)amino)-7H-pyrrolo [2,3-d]pyrimidin-5-yl)pyrazolo [1,5-a]pyridine-3-carboxamide [1327];
N-(2-fluoro-2-methylpropyl)-5-(2-(46-(4-me thylpiperazin-1-yl)pyridin-3-yl)methyl)amino)-7H-pyrrolo [2,3-d]pyrimidin-5-yl)pyrazolo [1,5-a]pyridine-3-carboxamide [1328];
N-(2-fluoro-2-methylpropyl)-5-(2-(quinolin-6-yl)-7H-pyrrolo [2,3-d] pyrimidin-5-yl)pyrazolo [1,5-a] pyridine-3-carboxamide [1329];
N-(2-fluoro-2-methylpropyl)-5-(4-(1-methyl-1H-pyrazol-4-yl)-7H-pyrrolo [2,3-d]pyrimidin-5-yl)pyrazolo [1,5-a]pyridine-3-carboxamide [1330];
N-(2-fluoro-2-methylpropyl)-5-(4-((trans-4-morpholinocyclohexyl)amino)-7H-pyrrolo [2,3-d]pyrimidin-5-yl)pyrazolo [1,5-a]pyridine-3-carboxamide [1331];
(R)-5-(2-isobutyl-7H-pyrrolo [2,3-d] pyrimidin-5-yl)-N-(1,1,1-trifluoropropan-2-yl)pyrazolo [1,5-a] pyridine-3-carboxamide [1333];
(R)-N-(1,1,1-trifluoropropan-2-yl)-5-(2-(3,3,3-trifluoropropyl)-7H-pyrrolo [2,3-d] pyrimidin-5-yl)pyrazolo [1,5-a]pyridine-3-carboxamide [1334];
(R)-5-(2-cyclopropyl-7H-pyrrolo [2,3-d]pyrimidin-5-yl)-N-(1,1,1-trifluoropropan-2-yl)pyrazolo [1,5-a]pyridine-3-carboxamide [1335];
(R)-5-(2-cyclobutyl-7H-pyrrolo [2,3-d]pyrimidin-5-yl)-N-(1,1,1-trifluoropropan-2-yl)pyrazolo [1,5-a]pyridine-3-carboxamide [1336];
(R)-5-(2-(cyclobutylmethyl)-7H-pyrrolo [2,3-d]pyrimidin-5-yl)-N-(1,1,1-trifluoropropan-2-yl)pyrazolo [1,5-a]pyridine-3-carboxamide [1337];
(R)-5-(2-amino-7H-pyrrolo [2,3-d]pyrimidin-5-yl)-N-(1,1,1-trifluoropropan-2-yl)pyrazolo [1,5-a] pyridine-3-carboxamide [1338];
(R)-5-(2-(methylamino)-7H-pyrrolo [2,3-d] pyrimidin-5-yl)-N-(1,1,1-trifluoropropan-2-yl)pyrazolo [1,5-a]pyridine-3-carboxamide [1339];
(R)-5-(2-(ethylamino)-7H-pyrrolo [2,3-d]pyrimidin-5-yl)-N-(1,1,1-trifluoropropan-2-yl)pyrazolo [1,5-a]pyridine-3-carboxamide [1340];
(R)-5-(2-(isopropylamino)-7H-pyrrolo [2,3-d]pyrimidin-5-yl)-N-(1,1,1-trifluoropropan-2-yl)pyrazolo [1,5-a]pyridine-3-carboxamide [1341];
(R)-5-(2-(isobutylamino)-7H-pyrrolo [2,3-d]pyrimidin-5-yl)-N-(1,1,1-trifluoropropan-2-yl)pyrazolo [1,5-a]pyridine-3-carboxamide [1342];
(R)-5-(2-(neopentylamino)-7H-pyrrolo [2,3-d] pyrimidin-5-yl)-N-(1,1,1-trifluoropropan-2-yl)pyrazolo [1,5-a]pyridine-3-carboxamide [1343];
5-(2-(((S)-sec-butyl)amino)-7H-pyrrolo [2,3-d]pyrimidin-5-yl)-N-((R)-1,1,1-trifluoropropan-2-yl)pyrazolo [1,5-a]pyridine-3-carboxamide [1344];
(R)-5-(2-((2-fluoro-2-methylpropyl)amino)-7H-pyrrolo [2,3-d]pyrimidin-5-yl)-N-(1,1,1-trifluoropropan-2-yl)pyrazolo [1,5-a]pyridine-3-carboxamide [1345];
(R)-5-(2-((2,2-difluoropropyl)amino)-7H-pyrrolo [2,3-d]pyrimidin-5-yl)-N-(1,1,1-trifluoropropan-2-yl)pyrazolo[1,5-a]pyridine-3-carboxamide [1346];
(R)-5-(2-((2,2,2-trifluoroethyl)amino)-7H-pyrrolo [2,3-d]pyrimidin-5-yl)-N-(1,1,1-trifluoropropan-2-yl)pyrazolo [1,5-a]pyridine-3-carboxamide [1347];
N-((R)-1,1,1-trifluoropropan-2-yl)-5-(2-(((R)-1,1,1-trifluoropropan-2-yl)amino)-7H-pyrrolo [2,3-d]pyrimidin-5-yl)pyrazolo [1,5-a]pyridine-3-carboxamide [1348];
N-((R)-1,1,1-trifluoropropan-2-yl)-5-(2-(((S)-1,1,1-trifluoropropan-2-yl)amino)-7H-pyrrolo [2,3-d]pyrimidin-5-yl)pyrazolo [1,5-a]pyridine-3-carboxamide [1349];
(R)-N-(1,1,1-trifluoropropan-2-yl)-5-(2-((3,3,3-trifluoropropyl)amino)-7H-pyrrolo [2,3-d] pyrimidin-5-yl)pyrazolo [1,5-a]pyridine-3-carboxamide [1350];
5-(2-(((S)-1-methoxypropan-2-yl)amino)-7H-pyrrolo [2,3-d]pyrimidin-5-yl)-N-((R)-1,1,1-trifluoropropan-2-yl)pyrazolo [1,5-a]pyridine-3-carboxamide [1351];
(R)-5-(2-ethoxy-7H-pyrrolo [2,3-d] pyrimidin-5-yl)-N-(1,1,1-trifluoropropan-2-yl)pyrazolo [1,5-a] pyridine-3-carboxamide [1352];
(R)-5-(2-(((1-fluorocyclopropyl)methyl)amino)-7H-pyrrolo [2,3-d]pyrimidin-5-yl)-N-(1,1,1-trifluoropropan-2-yl)pyrazolo [1,5-a]pyridine-3-carboxamide [1353];
5-(2-(((R)-1-cyclopropylethyl)amino)-7H-pyrrolo [2,3-d]pyrimidin-5-yl)-N-((R)-1, 1, 1-trifluoropropan-2-yl)pyrazolo [1,5-a]pyridine-3-carboxamide [1354];
(R)-5-(2-(((1-methylcyclopropyl)methyl)amino)-7H-pyrrolo [2,3-d]pyrimidin-5-yl)-N-(1, 1, 1-trifluoropropan-2-yl)pyrazolo [1,5-a]pyridine-3-carboxamide [1355];
(R)-5-(2-(((1-(trifluoromethyl)cyclopropyl)methyl)amino)-7H-pyrrolo [2,3-d]pyrimidin-5-yl)-N-(1, 1, 1-trifluoropropan-2-yl)pyrazolo [1,5-a]pyridine-3-carboxamide [1356];
(R)-5-(2-((2-cyclopropyl-2,2-difluoroethyl)amino)-7H-pyrrolo [2,3-d]pyrimidin-5-yl)-N-(1, 1, 1-trifluoropropan-2-yl)pyrazolo [1,5-a]pyridine-3-carboxamide [1357];
(R)-5-(2-((3,3-difluorocyclobutyl)amino)-7H-pyrrolo [2,3-d]pyrimidin-5-yl)-N-(1, 1, 1-trifluoropropan-2-yl)pyrazolo [1,5-a]pyridine-3-carboxamide [1358];
(R)-5-(2-(((1-fluorocyclobutyl)methyl)amino)-7H-pyrrolo [2,3-d]pyrimidin-5-yl)-N-(1, 1, 1-trifluoropropan-2-yl)pyrazolo [1,5-a]pyridine-3-carboxamide [1359];
(R)-5-(2-((4,4-difluorocyclohexyl)amino)-7H-pyrrolo [2,3-d]pyrimidin-5-yl)-N-(1, 1, 1-trifluoropropan-2-yl)pyrazolo [1,5-a]pyridine-3-carboxamide [1360];
5-(2-((cis-4-methoxycyclohexyl)amino)-7H-pyrrolo [2,3-d]pyrimidin-5-yl)-N-((R)-1, 1, 1-trifluoropropan-2-yl)pyrazolo [1,5-a]pyridine-3-carboxamide [1361];
(R)-5-(2-(((1-fluorocyclohexyl)methyl)amino)-7H-pyrrolo [2,3-d]pyrimidin-5-yl)-N-(1, 1, 1-trifluoropropan-2-yl)pyrazolo [1,5-a]pyridine-3-carboxamide [1362];
(R)-5-(2-(((4,4-difluorocyclohexyl)methyl)amino)-7H-pyrrolo [2,3-d]pyrimidin-5-yl)-N-(1, 1, 1-trifluoropropan-2-yl)pyrazolo [1,5-a]pyridine-3-carboxamide [1363];
(R)-5-(2-((1-methylpiperidin-4-yl)amino)-7H-pyrrolo [2,3-d]pyrimidin-5-yl)-N-(1, 1, 1-trifluoropropan-2-yl)pyrazolo [1,5-a]pyridine-3-carboxamide [1364];
(R)-5-(2-(((1-methylpiperidin-4-yl)methyl)amino)-7H-pyrrolo [2,3-d]pyrimidin-5-yl)-N-(1, 1, 1-trifluoropropan-2-yl)pyrazolo [1,5-a]pyridine-3-carboxamide [1365];
(R)-5-(2-((tetrahydro-2H-pyran-4-yl)amino)-7H-pyrrolo [2,3-d]pyrimidin-5-yl)-N-(1, 1, 1-trifluoropropan-2-yl)pyrazolo [1,5-a]pyridine-3-carboxamide [1366];
(R)-5-(2-((3-(4-methylpiperazin-1-yl)phenyl)amino)-7H-pyrrolo [2,3-d]pyrimidin-5-yl)-N-(1, 1, 1-trifluoropropan-2-yl)pyrazolo [1,5-a]pyridine-3-carboxamide [1367];
(R)-5-(2-(pyridin-4-ylamino)-7H-pyrrolo [2,3-d]pyrimidin-5-yl)-N-(1, 1, 1-trifluoropropan-2-yl)pyrazolo [1,5-a]pyridine-3-carboxamide [1368];
(R)-5-(2-((2-(4-methylpiperazin-1-yl)pyridin-4-yl)amino)-7H-pyrrolo [2,3-d]pyrimidin-5-yl)-N-(1, 1, 1-trifluoropropan-2-yl)pyrazolo [1,5-a]pyridine-3-carboxamide [1369];
(R)-5-(2-((6-(4-methylpiperazin-1-yl)pyridin-3-yl)amino)-7H-pyrrolo [2,3-d]pyrimidin-5-yl)-N-(1, 1, 1-trifluoropropan-2-yl)pyrazolo [1,5-a]pyridine-3-carboxamide [1370];
(R)-5-(2-(((6-(4-methylpiperazin-1-yl)pyridin-3-yl)methyl)amino)-7H-pyrrolo [2,3-d]pyrimidin-5-yl)-N-(1,1,1-trifluoropropan-2-yl)pyrazolo [1,5-a]pyridine-3-carboxamide [1371];
(R)-5-(2-(quinolin-6-yl)-7H-pyrrolo [2,3-d] pyrimidin-5-yl)-N-(1,1,1-trifluoropropan-2-yl)pyrazolo [1,5-a]pyridine-3-carboxamide [1372];
(R)-5-(4-(1-methyl-1H-pyrazol-4-yl)-7H-pyrrolo [2,3-d]pyrimidin-5-yl)-N-(1,1,1-trifluoropropan-2-yl)pyrazolo [1,5-a]pyridine-3-carboxamide [1373];
5-(4-((trans-4-morpholinocyclohexyl)amino)-7H-pyrrolo [2,3-d] pyrimidin-5-yl)-N-((R)-1,1,1-trifluoropropan-2-yl)pyrazolo [1,5-a]pyridine-3-carboxamide [1374];
N-(3,3-difluorocyclobutyl)-5-(2-i sobutyl-7H-pyrrolo [2,3-d]pyrimidin-5-yl)pyrazolo [1,5-a] pyridine-3-carboxamide [1376];
N-(3,3-difluorocyclobutyl)-5-(2-(3,3,3-trifluoropropyl)-7H-pyrrolo [2,3-d]pyrimidin-5-yl)pyrazolo [1,5-a]pyridine-3-carboxamide [1377];
5-(2-cyclopropyl-7H-pyrrolo [2,3-d]pyrimidin-5-yl)-N-(3,3-difluorocyclobutyppyrazolo [1,5-a] pyridine-3-carboxamide [1378];
5-(2-cyclopropyl-7H-pyrrolo [2,3-d]pyrimidin-5-yl)-N-(3,3-difluorocyclobutyl)-N-methylpyrazolo [1,5-a]pyridine-3-carboxamide [1379];
5-(2-cyclobutyl-7H-pyrrolo [2,3-d]pyrimidin-5-yl)-N-(3,3-difluorocyclobutyl)pyrazolo [1,5-a] pyridine-3-carboxamide [1380];
5-(2-(cyclobutylmethyl)-7H-pyrrolo [2,3-d]pyrimidin-5-yl)-N-(3,3-difluorocyclobutyl)pyrazolo [1,5-a] pyridine-3-carboxamide [1381];
5-(2-amino-7H-pyrrolo [2,3-d] pyrimidin-5-yl)-N-(3,3-difluorocyclobutyl)pyrazolo [1,5-a] pyridine-3-carboxamide [1382];
N-(3,3-difluorocyclobutyl)-5-(2-(methylamino)-7H-pyrrolo [2,3-d]pyrimidin-5-yl)pyrazolo [1,5-a] pyridine-3-carboxamide [1383];
N-(3,3-difluorocyclobutyl)-5-(2-(ethylamino)-7H-pyrrolo [2,3-d]pyrimidin-5-yl)pyrazolo [1,5-a] pyridine-3-carboxamide [1384];
N-(3,3-difluorocyclobutyl)-5-(2-(i sop ropylamino)-7H-pyrrolo [2,3-d]pyrimidin-5-yl)pyrazolo [1,5-a] pyridine-3-carboxamide [1385];
N-(3,3-difluorocyclobutyl)-5-(2-(i sobutylamino)-7H-pyrrolo [2,3-d]pyrimidin-5-yl)pyrazolo [1,5-a] pyridine-3-carboxamide [1386];
N-(3,3-difluorocyclobutyl)-5-(2-(neopentylamino)-7H-pyrrolo [2,3-d]pyrimidin-5-yl)pyrazolo [1,5-a]pyridine-3-carboxamide [1387];
(S)-5-(2-(sec-butylamino)-7H-pyrrolo [2,3-d] pyrimidin-5-yl)-N-(3,3-difluorocyclobutyl)pyrazolo [1,5-a] pyridine-3-carboxamide [1388];
N-(3,3-difluorocyclobutyl)-5-(2-((2-fluoro-2-methylpropyl)amino)-7H-pyrrolo [2,3-d] pyrimidin-5-yl)pyrazolo [1,5-a]pyridine-3-carboxamide [1389];
N-(3,3-difluorocyclobutyl)-5-(2-((2,2-difluoropropyl)amino)-7H-pyrrolo [2,3-d]pyrimidin-5-yl)pyrazolo [1,5-a]pyridine-3-carboxamide [1390];
N-(3,3-difluorocyclobutyl)-5-(2-((2,2,2-trifluoroethyl)amino)-7H-pyrrolo [2,3-d]pyrimidin-5-yl)pyrazolo [1,5-a]pyridine-3-carboxamide [1391];
(R)-N-(3,3-difluorocyclobutyl)-5-(2-((1,1,1-trifluoropropan-2-yl)amino)-7H-pyrrolo [2,3-d]pyrimidin-5-yl)pyrazolo [1,5-a]pyridine-3-carboxamide [1392];
(S)-N-(3,3-difluorocyclobutyl)-5-(2-((1,1,1-trifluoropropan-2-yl)amino)-7H-pyrrolo [2,3-d]pyrimidin-5-yl)pyrazolo [1,5-a]pyridine-3-carboxamide [1393];
N-(3,3-difluorocyclobutyl)-5-(2-((3,3,3-trifluoropropyl)amino)-7H-pyrrolo [2,3-d]pyrimidin-5-yl)pyrazolo [1,5-a]pyridine-3-carboxamide [1394];
(S)-N-(3,3-difluorocyclobutyl)-5-(2-((l-methoxypropan-2-yl)amino)-7H-pyrrolo [2,3-d]pyrimidin-5-yl)pyrazolo [1,5-a]pyridine-3-carboxamide [1395];
N-(3,3-difluorocyclobutyl)-5-(2-ethoxy-7H-pyrrolo [2,3-d] pyrimidin-5-yl)pyrazolo [1,5-a] pyridine-3-carboxamide [1396];
N-(3,3-difluorocyclobutyl)-5-(2-(((1-fluorocyclopropyl)methyl)amino)-7H-pyrrolo [2,3-d]pyrimidin-5-yl)pyrazolo [1,5-a]pyridine-3-carboxamide [1397];
(R)-5-(2-((l-cyclopropylethyl)amino)-7H-pyrrolo [2,3-d]pyrimidin-5-yl)-N-(3,3-difluorocyclobutyl)pyrazolo [1,5-a] pyridine-3-carboxamide [1398];
N-(3,3-difluorocyclobutyl)-5-(2-(((l-methylcyclopropyl)me thyl)amino)-7H-pyrrolo [2,3-d]pyrimidin-5-yl)pyrazolo [1,5-a]pyridine-3-carboxamide [1399];
N-(3,3-difluorocyclobutyl)-5-(2-(((1-(trifluorome thyl)cyclopropyl)me thyl)amino)-7H-pyrrolo [2,3-d]pyrimidin-5-yl)pyrazolo [1,5-a]pyridine-3-carboxamide [1400];
5-(2-((2-cyclopropyl-2,2-difluoroethyl)amino)-7H-pyrrolo [2,3-d]pyrimidin-5-yl)-N-(3,3-difluorocyclobutyl)pyrazolo [1,5-a] pyridine-3-carboxamide [1401];
N-(3,3-difluorocyclobutyl)-5-(2-((3,3-difluorocyclobutypamino)-7H-pyrrolo [2,3-d]pyrimidin-5-yl)pyrazolo [1,5-a]pyridine-3-carboxamide [1402];
N-(3,3-difluorocyclobutyl)-5-(2-(((1-fluorocyclobu tyl)methyl)amino)-7H-pyrrolo [2,3-d]pyrimidin-5-yl)pyrazolo [1,5-a]pyridine-3-carboxamide [1403];
N-(3,3-difluorocyclobutyl)-5-(2-((4,4-difluorocyclohexyl)amino)-7H-pyrrolo [2,3-d]pyrimidin-5-yl)pyrazolo [1,5-a]pyridine-3-carboxamide [1404];
N-(3,3-difluorocyclobutyl)-5-(2-((cis-4-methoxycyclohexyl)amino)-7H-pyrrolo [2,3-d]pyrimidin-5-yl)pyrazolo[1,5-a]pyridine-3-carboxamide [1405];
N-(3,3-difluorocyclobutyl)-5-(2-(((1-fluorocyclohexyl)me thyl)amino)-7H-pyrrolo [2,3-d]pyrimidin-5-yl)pyrazolo [1,5-a]pyridine-3-carboxamide [1406];
N-(3,3-difluorocyclobutyl)-5-(2-4(4,4-difluorocyclohexyl)methypamino)-7H-pyrrolo [2,3-d]pyrimidin-5-yl)pyrazolo [1,5-a]pyridine-3-carboxamide [1407];
N-(3,3-difluorocyclobutyl)-5-(2-((1-methylpiperidin-4-yl)amino)-7H-pyrrolo [2,3-d]pyrimidin-5-yl)pyrazolo [1,5-a]pyridine-3-carboxamide [1408];
N-(3,3-difluorocyclobutyl)-5-(2-(((1-methylpiperidin-4-yl)me thyl)amino)-7H-pyrrolo [2,3-d]pyrimidin-5-yl)pyrazolo [1,5-a]pyridine-3-carboxamide [1409];
N-(3,3-difluorocyclobutyl)-5-(2-((tetrahydro-2H-pyran-4-yl)amino)-7H-pyrrolo [2,3-d]pyrimidin-5-yl)pyrazolo[1,5-a]pyridine-3-carboxamide [1410];
N-(3,3-difluorocyclobutyl)-5-(2-43-(4-me thylpiperazin-1-yl)phenyl)amino)-7H-pyrrolo [2,3-d]pyrimidin-5-yl)pyrazolo [1,5-a]pyridine-3-carboxamide [1411];
N-(3,3-difluorocyclobutyl)-5-(2-(pyridin-4-ylamino)-7H-pyrrolo [2,3-d]pyrimidin-5-yl)pyrazolo [1,5-a]pyridine-3-carboxamide [1412];
N-(3,3-difluorocyclobutyl)-5-(2-42-(4-methylpiperazin-1-yl)pyridin-4-yl)amino)-7H-pyrrolo [2,3-d]pyrimidin-5-yl)pyrazolo [1,5-a]pyridine-3-carboxamide [1413];
N-(3,3-difluorocyclobutyl)-5-(2-46-(4-methylpiperazin-1-yl)pyridin-3-yl)amino)-7H-pyrrolo [2,3-d]pyrimidin-5-yl)pyrazolo [1,5-a]pyridine-3-carboxamide [1414];
N-(3,3-difluorocyclobutyl)-5-(2-(46-(4-methylpiperazin-1-yl)pyridin-3-yl)methyl)amino)-7H-pyrrolo [2,3-d]pyrimidin-5-yl)pyrazolo [1,5-a]pyridine-3-carboxamide [1415];
N-(3,3-difluorocyclobutyl)-5-(2-(quinolin-6-yl)-7H-pyrrolo [2,3-d]pyrimidin-5-yl)pyrazolo [1,5-a] pyridine-3-carboxamide [1416];
N-(3,3-difluorocyclobutyl)-5-(4-(1-methyl-1H-pyrazol-4-yl)-7H-pyrrolo [2,3-d] pyrimidin-5-yl)pyrazolo [1,5-a]pyridine-3-carboxamide [1417];
N-(3,3-difluorocyclobutyl)-5-(4-((trans-4-morpholinocyclohexyl)amino)-7H-pyrrolo [2,3-d]pyrimidin-5-yl)pyrazolo [1,5-a]pyridine-3-carboxamide [1418];
N-((3,3-difluorocyclobutypmethyl)-5-(2-isobutyl-7H-pyrrolo [2,3-d]pyrimidin-5-yl)pyrazolo [1,5-a] pyridine-3-carboxamide [1420];
N-((3,3-difluorocyclobutypmethyl)-5-(2-(3,3,3-trifluoropropyl)-7H-pyrrolo [2,3-d] pyrimidin-5-yl)pyrazolo [1,5-a]pyridine-3-carboxamide [1421];
5-(2-cyclopropyl-7H-pyrrolo [2,3-d]pyrimidin-5-yl)-N-((3,3-difluorocyclobutyl)methyl)pyrazolo [1,5-a]pyridine-3-carboxamide [1422];
5-(2-cyclobutyl-7H-pyrrolo [2,3-d] pyrimidin-5-yl)-N-((3,3-difluorocyclobutyl)methyl)pyrazolo [1,5-a]pyridine-3-carboxamide [1423];
5-(2-(cyclobutylmethyl)-7H-pyrrolo [2,3-d]pyrimidin-5-yl)-N-((3,3-difluorocyclobutypmethyl)pyrazolo [1,5-a]pyridine-3-carboxamide [1424];
5-(2-amino-7H-pyrrolo [2,3-d] pyrimidin-5-yl)-N-((3,3-difluorocyclobutyl)methyl)pyrazolo [1,5-a] pyridine-3-carboxamide [1425];
N-((3,3-difluorocyclobutyl)methyl)-5-(2-(methylamino)-7H-pyrrolo [2,3-d] pyrimidin-5-yl)pyrazolo [1,5-a]pyridine-3-carboxamide [1426];
N-((3,3-difluorocyclobutyl)methyl)-5-(2-(ethylamino)-7H-pyrrolo [2,3-d]pyrimidin-5-yl)pyrazolo [1,5-a]pyridine-3-carboxamide [1427];
N-((3,3-difluorocyclobutyl)methyl)-5-(2-(isopropylamino)-7H-pyrrolo [2,3-d]pyrimidin-5-yl)pyrazolo [1,5-a]pyridine-3-carboxamide [1428];
N-((3,3-difluorocyclobutyl)methyl)-5-(2-(isobutylamino)-7H-pyrrolo [2,3-d] pyrimidin-5-yl)pyrazolo [1,5-a]pyridine-3-carboxamide [1429];
N-((3,3-difluorocyclobutyl)methyl)-5-(2-(neopentylamino)-7H-pyrrolo [2,3-d] pyrimidin-5-yl)pyrazolo [1,5-a]pyridine-3-carboxamide [1430];
(S)-5-(2-(sec-butylamino)-7H-pyrrolo [2,3-d] pyrimidin-5-yl)-N-((3,3-difluorocyclobutyl)methyl)pyrazolo [1,5-a]pyridine-3-carboxamide [1431];
N-((3,3-difluorocyclobutyl)methyl)-5-(2-((2-fluoro-2-methylpropyl)amino)-7H-pyrrolo [2,3-d]pyrimidin-5-yl)pyrazolo [1,5-a]pyridine-3-carboxamide [1432];
N-((3,3-difluorocyclobutyl)methyl)-5-(2-((2,2-difluoropropyl)amino)-7H-pyrrolo [2,3-d]pyrimidin-5-yl)pyrazolo [1,5-a]pyridine-3-carboxamide [1433];
N-((3,3-difluorocyclobutyl)methyl)-5-(2-((2,2,2-trifluoroethyl)amino)-7H-pyrrolo [2,3-d] pyrimidin-5-yl)pyrazolo [1,5-a]pyridine-3-carboxamide [1434];
(R)-N-((3,3-difluorocyclobutyl)methyl)-5-(2-((1,1,1-trifluoropropan-2-yl)amino)-7H-pyrrolo [2,3-d] pyrimidin-5-yl)pyrazolo [1,5-a]pyridine-3-carboxamide [1435];
(S)-N-((3,3-difluorocyclobutyl)methyl)-5-(2-((1,1,1-trifluoropropan-2-yl)amino)-7H-pyrrolo [2,3-d] pyrimidin-5-yl)pyrazolo [1,5-a]pyridine-3-carboxamide [1436];
N-((3,3-difluorocyclobutyl)methyl)-5-(2-((3,3,3-trifluoropropyl)amino)-7H-pyrrolo [2,3-d]pyrimidin-5-yl)pyrazolo [1,5-a]pyridine-3-carboxamide [1437];
(S)-N-((3,3-difluorocyclobutyl)methyl)-5-(2-((l-methoxypropan-2-yl)amino)-7H-pyrrolo [2,3-d]pyrimidin-5-yl)pyrazolo [1,5-a]pyridine-3-carboxamide [1438];
N-((3,3-difluorocyclobutyl)methyl)-5-(2-ethoxy-7H-pyrrolo [2,3-d]pyrimidin-5-yl)pyrazolo [1,5-a] pyridine-3-carboxamide [1439];
N-((3,3-difluorocyclobutyl)methyl)-5-(2-(((1-fluorocyclopropyl)methyl)amino)-7H-pyrrolo [2,3-d]pyrimidin-5-yl)pyrazolo [1,5-a]pyridine-3-carboxamide [1440];
(R)-5-(2-((l-cyclopropylethyl)amino)-7H-pyrrolo [2,3-d]pyrimidin-5-yl)-N-((3,3-difluorocyclobutyl)methyl)pyrazolo [1,5-a]pyridine-3-carboxamide [1441];
N-((3,3-difluorocyclobutyl)methyl)-5-(2-(((l-methylcyclopropyl)methyl)amino)-7H-pyrrolo [2,3-d]pyrimidin-5-yl)pyrazolo [1,5-a]pyridine-3-carboxamide [1442];
N-((3,3-difluorocyclobutyl)methyl)-5-(2-(((1-(trifluoromethyl)cyclopropyl)methyl)amino)-7H-pyrrolo [2,3-d]pyrimidin-5-yl)pyrazolo [1,5-a]pyridine-3-carboxamide [1443];
5-(2-((2-cyclopropyl-2,2-difluoroethypamino)-7H-pyrrolo [2,3-d]pyrimidin-5-yl)-N-((3,3-difluorocyclobutyl)methyl)pyrazolo [1,5-a]pyridine-3-carboxamide [1444];
5-(2-((3,3-difluorocyclobutyl)amino)-7H-pyrrolo [2,3-d]pyrimidin-5-yl)-N-((3,3-difluorocyclobutyl)methyl)pyrazolo [1,5-a]pyridine-3-carboxamide [1445];
N-((3,3-difluorocyclobutyl)methyl)-5-(2-(((1-fluorocyclobutyl)me thyl)amino)-7H-pyrrolo [2,3-d]pyrimidin-5-yl)pyrazolo [1,5-a]pyridine-3-carboxamide [1446];
N-((3,3-difluorocyclobutyl)methyl)-5-(2-((4,4-difluorocyclohexyl)amino)-7H-pyrrolo [2,3-d]pyrimidin-5-yl)pyrazolo [1,5-a]pyridine-3-carboxamide [1447];
N-((3,3-difluorocyclobutyl)methyl)-5-(2-((cis-4-methoxycyclohexyl)amino)-7H-pyrrolo [2,3-d]pyrimidin-5-yl)pyrazolo [1,5-a]pyridine-3-carboxamide [1448];
N-((3,3-difluorocyclobutyl)methyl)-5-(2-(((1-fluorocyclohexyl)methyl)amino)-7H-pyrrolo [2,3-d]pyrimidin-5-yl)pyrazolo [1,5-a]pyridine-3-carboxamide [1449];
N-((3,3-difluorocyclobutyl)methyl)-5-(2-(((4,4-difluorocyclohexyl)methyl)amino)-7H-pyrrolo [2,3-d]pyrimidin-5-yl)pyrazolo [1,5-a]pyridine-3-carboxamide [1450];
N-((3,3-difluorocyclobutyl)methyl)-5-(2-((l-methylpiperidin-4-yl)amino)-7H-pyrrolo [2,3-d]pyrimidin-5-yl)pyrazolo [1,5-a]pyridine-3-carboxamide [1451];
N-((3,3-difluorocyclobutyl)methyl)-5-(2-(((l-methylpiperidin-4-yl)methyl)amino)-7H-pyrrolo [2,3-d]pyrimidin-5-yl)pyrazolo [1,5-a]pyridine-3-carboxamide [1452];
N-((3,3-difluorocyclobutyl)methyl)-5-(2-((tetrahydro-2H-pyran-4-yl)amino)-7H-pyrrolo [2,3-d]pyrimidin-5-yl)pyrazolo [1,5-a]pyridine-3-carboxamide [1453];
N-((3,3-difluorocyclobutypmethyl)-5-(2-43-(4-methylpiperazin-1-yl)phenyl)amino)-7H-pyrrolo [2,3-d]pyrimidin-5-yl)pyrazolo [1,5-a]pyridine-3-carboxamide [1454];
N-((3,3-difluorocyclobutyl)methyl)-5-(2-(pyridin-4-ylamino)-7H-pyrrolo [2,3-d] pyrimidin-5-yl)pyrazolo [1,5-a]pyridine-3-carboxamide [1455];
N-((3,3-difluorocyclobutypmethyl)-5-(2-42-(4-methylpiperazin-1-yl)pyridin-4-yl)amino)-7H-pyrrolo [2,3-d]pyrimidin-5-yl)pyrazolo [1,5-a]pyridine-3-carboxamide [1456];
N-((3,3-difluorocyclobutypmethyl)-5-(2-46-(4-methylpiperazin-1-yl)pyridin-3-yl)amino)-7H-pyrrolo [2,3-d]pyrimidin-5-yl)pyrazolo [1,5-a]pyridine-3-carboxamide [1457];
N-((3-difluorocyclobutypmethyl)-5-(2-(46-(4-me thylpiperazin-1-yl)pyridin-3-yl)methyl)amino)-7H-pyrrolo [2,3-d]pyrimidin-5-yl)pyrazolo [1,5-a]pyridine-3-carboxamide [1458];
N-((3-difluorocyclobutypmethyl)-5-(2-(quinolin-6-yl)-7H-pyrrolo [2,3-d]pyrimidin-5-yl)pyrazolo [1,5-a]pyridine-3-carboxamide [1459];
N-((3,3-difluorocyclobutyl)methyl)-5-(4-(1-methyl-1H-pyrazol-4-yl)-7H-pyrrolo [2,3-d]pyrimidin-5-yl)pyrazolo [1,5-a]pyridine-3-carboxamide [1460];
N-((3-difluorocyclobutypmethyl)-5-(4-((trans-4-morpholinocyclohexyl)amino)-7H-pyrrolo [2,3-d]pyrimidin-5-yl)pyrazolo [1,5-a]pyridine-3-carboxamide [1461];
5-(2-i sobutyl-7H-pyrrolo [2,3-d]pyrimidin-5-yl)-N-(trans-4-methoxycyclohexyl)pyrazolo [1,5-a] pyridine-3-carboxamide [1463];
N-(trans-4-methoxycyclohexyl)-5-(243,3,3-trifluoropropyl)-7H-pyrrolo [2,3-d]pyrimidin-5-yl)pyrazolo [1,5-a]pyridine-3-carboxamide [1464];
5-(2-cyclopropyl-7H-pyrrolo [2,3-d]pyrimidin-5-yl)-N-(trans-4-methoxycyclohexyl)pyrazolo [1,5-a] pyridine-3-carboxamide [1465];
5-(2-cyclobutyl-7H-pyrrolo [2,3-d]pyrimidin-5-yl)-N-(trans-4-methoxycyclohexyl)pyrazolo [1,5-a] pyridine-3-carboxamide [1466];
5-(2-(cyclobutylmethyl)-7H-pyrrolo [2,3-d]pyrimidin-5-yl)-N-(trans-4-methoxycyclohexyl)pyrazolo [1,5-a] pyridine-3-carboxamide [1467];
5-(2-amino-7H-pyrrolo [2,3-d]pyrimidin-5-yl)-N-(trans-4-methoxycyclohexyl)pyrazolo [1,5-a] pyridine-3-carboxamide [1468];
N-(trans-4-methoxycyclohexyl)-5-(2-(methylamino)-7H-pyrrolo [2,3-d]pyrimidin-5-yl)pyrazolo [1,5-a]pyridine-3-carboxamide [1469];
5-(2-(ethylamino)-7H-pyrrolo [2,3-d]pyrimidin-5-yl)-N-(trans-4-methoxycyclohexyl)pyrazolo [1,5-a] pyridine-3-carboxamide [1470];
5-(2-(i sop ropylamino)-7H-pyrrolo [2,3-d]pyrimidin-5-yl)-N-(trans-4-methoxycyclohexyl)pyrazolo [1,5-a] pyridine-3-carboxamide [1471];
5-(2-(i sobutylamino)-7H-pyrrolo [2,3-d]pyrimidin-5-yl)-N-(trans-4-methoxycyclohexyl)pyrazolo [1,5-a] pyridine-3-carboxamide [1472];
N-(trans-4-methoxycyclohexyl)-5-(2-(neopentylamino)-7H-pyrrolo [2,3-d]pyrimidin-5-yl)pyrazolo [1,5-a]pyridine-3-carboxamide [1473];
5-(2-(((S)-se c-butyl)amino)-7H-pyrrolo [2,3-d]pyrimidin-5-yl)-N-(trans-4-methoxycyclohexyl)pyrazolo [1,5-a] pyridine-3-carboxamide [1474];
5-(2-((2-fluoro-2-methylpropyl)amino)-7H-pyrrolo [2,3-d]pyrimidin-5-yl)-N-(trans-4-methoxycyclohexyl)pyrazolo [1,5-a] pyridine-3-carboxamide [1475];
5-(2-((2,2-difluoropropyl)amino)-7H-pyrrolo [2,3-d] pyrimidin-5-yl)-N-(trans-4-methoxycyclohexyl)pyrazolo [1,5-a] pyridine-3-carboxamide [1476];
N-(trans-4-methoxycyclohexyl)-5-(2-((2,2,2-trifluoroethypamino)-7H-pyrrolo [2,3-d] pyrimidin-5-yl)pyrazolo [1,5-a]pyridine-3-carboxamide [1477];
N-(trans-4-methoxycyclohexyl)-5-(2-4(R)-1,1,1-trifluoropropan-2-yl)amino)-7H-pyrrolo [2,3-d]pyrimidin-5-yl)pyrazolo [1,5-a]pyridine-3-carboxamide [1478];
N-(trans-4-methoxycyclohexyl)-5-(2-4(S)-1,1,1-trifluoropropan-2-yl)amino)-7H-pyrrolo [2,3-d]pyrimidin-5-yl)pyrazolo [1,5-a]pyridine-3-carboxamide [1479];
N-(trans-4-methoxycyclohexyl)-5-(2-((3,3,3-trifluoropropyl)amino)-7H-pyrrolo [2,3-d]pyrimidin-5-yl)pyrazolo[1,5-a]pyridine-3-carboxamide [1480];
N-(trans-4-methoxycyclohexyl)-5-(2-4(S)-1-methoxypropan-2-yl)amino)-7H-pyrrolo [2,3-d]pyrimidin-5-yl)pyrazolo [1,5-a]pyridine-3-carboxamide [1481];
5-(2-ethoxy-7H-pyrrolo [2,3-d] pyrimidin-5-yl)-N-(trans-4-methoxycyclohexyl)pyrazolo [1,5-a] pyridine-3-carboxamide [1482];
5-(2-(((1-fluorocyclopropyl)methyl)amino)-7H-pyrrolo [2,3-d]pyrimidin-5-yl)-N-(trans-4-methoxycyclohexyl)pyrazolo [1,5-a] pyridine-3-carboxamide [1483];
5-(2-(((R)-1-cyclopropylethyl)amino)-7H-pyrrolo [2,3-d]pyrimidin-5-yl)-N-(trans-4-methoxycyclohexyl)pyrazolo [1,5-a] pyridine-3-carboxamide [1484];
N-(trans-4-methoxycyclohexyl)-5-(2-(((1-methylcyclopropyl)methyl)amino)-7H-pyrrolo [2,3-d]pyrimidin-5-yl)pyrazolo [1,5-a]pyridine-3-carboxamide [1485];
N-(trans-4-methoxycyclohexyl)-5-(2-(41-(trifluoromethyl)cyclopropyl)methyl)amino)-7H-pyrrolo [2,3-d]pyrimidin-5-yl)pyrazolo [1,5-a]pyridine-3-carboxamide [1486];
5-(2-((2-cyclopropyl-2,2-difluoroethyl)amino)-7H-pyrrolo [2,3-d]pyrimidin-5-yl)-N-(trans-4-methoxycyclohexyl)pyrazolo [1,5-a] pyridine-3-carboxamide [1487];
5-(2-((3,3-difluorocyclobutyl)amino)-7H-pyrrolo [2,3-d]pyrimidin-5-yl)-N-(trans-4-methoxycyclohexyl)pyrazolo [1,5-a] pyridine-3-carboxamide [1488];
5-(2-(((1-fluorocyclobutyl)methyl)amino)-7H-pyrrolo [2,3-d]pyrimidin-5-yl)-N-(trans-4-methoxycyclohexyl)pyrazolo [1,5-a] pyridine-3-carboxamide [1489];
5-(2-((4,4-difluorocyclohexyl)amino)-7H-pyrrolo [2,3-d]pyrimidin-5-yl)-N-(trans-4-methoxycyclohexyl)pyrazolo [1,5-a] pyridine-3-carboxamide [1490];
N-(trans-4-methoxycyclohexyl)-5-(2-((cis-4-methoxycyclohexyl)amino)-7H-pyrrolo [2,3-d]pyrimidin-5-yl)pyrazolo [1,5-a]pyridine-3-carboxamide [1491];
5-(2-(((1-fluorocyclohexyl)methyl)amino)-7H-pyrrolo [2,3-d]pyrimidin-5-yl)-N-(trans-4-methoxycyclohexyl)pyrazolo [1,5-a] pyridine-3-carboxamide [1492];
5-(2-(((4,4-difluorocyclohexyl)methyl)amino)-7H-pyrrolo[2,3-d]pyrimidin-5-yl)-N-(trans-4-methoxycyclohexyl)pyrazolo [1,5-a] pyridine-3-carboxamide [1493];
N-(trans-4-methoxycyclohexyl)-5-(2-((1-methylpiperidin-4-yl)amino)-7H-pyrrolo [2,3-d] pyrimidin-5-yl)pyrazolo [1,5-a] pyridine-3-carboxamide [1494];
N-(trans-4-methoxycyclohexyl)-5-(2-(((1-methylpiperidin-4-yl)methyl)amino)-7H-pyrrolo [2,3-d] pyrimidin-5-yl)pyrazolo [1,5-a] pyridine-3-carboxamide [1495];
N-(trans-4-methoxycyclohexyl)-5-(2-((tetrahydro-2H-pyran-4-yl)amino)-7H-pyrrolo [2,3-d] pyrimidin-5-yl)pyrazolo [1,5-a] pyridine-3-carboxamide [1496];
N-(trans-4-methoxycyclohexyl)-5-(2-43-(4-methylpiperazin-1-yl)phenyl)amino)-7H-pyrrolo [2,3-d]pyrimidin-5-yl)pyrazolo [1,5-a] pyridine-3-carboxamide [1497];
N-(trans-4-methoxycyclohexyl)-5-(2-(pyridin-4-ylamino)-7H-pyrrolo [2,3-d]pyrimidin-5-yl)pyrazolo [1,5-a]pyridine-3-carboxamide [1498];
N-(trans-4-methoxycyclohexyl)-5-(2-42-(4-methylpiperazin-1-yl)pyridin-4-yl)amino)-7H-pyrrolo [2,3-d]pyrimidin-5-yl)pyrazolo [1,5-a]pyridine-3-carboxamide [1499]; and
N-(trans-4-methoxycyclohexyl)-5-(2-46-(4-methylpiperazin-1-yl)pyridin-3-yl)amino)-7H-pyrrolo [2,3-d]pyrimidin-5-yl)pyrazolo [1,5-a]pyridine-3-carboxamide [1500]; or a pharmaceutically acceptable salt thereof.

26. The compound of claim 1, wherein the compound of Formula I is selected from the group consisting of:
N-(trans-4-methoxycyclohexyl)-5-(2-(46-(4-methylpiperazin-1-yl)pyridin-3-yl)methyl)amino)-7H-pyrrolo [2,3-d]pyrimidin-5-yl)pyrazolo [1,5-a] pyridine-3-carboxamide [1501];
N-(trans -4-methoxycyclohexyl)-5-(2-(quinolin-6-yl)-7H-pyrrolo [2,3-d]pyrimidin-5-yl)pyrazolo [1,5-a]pyridine-3-carboxamide [1502];
N-(trans-4-methoxycyclohexyl)-5-(4-(1-methyl-1H-pyrazol-4-yl)-7H-pyrrolo [2,3-d]pyrimidin-yl)pyrazolo [1,5-a]pyridine-3-carboxamide [1503];
N-(trans-4-methoxycyclohexyl)-5-(4-((trans-4-morpholinocyclohexyl)amino)-7H-pyrrolo [2,3-d] pyrimidin-5-yl)pyrazolo [1,5-a] pyridine-3-carboxamide [1504];
5-(2-isobutyl-7H-pyrrolo [2,3-d]pyrimidin-5-yl)-N-(1-methylpiperidin-4-yl)pyrazolo [1,5-a]pyridine-3-carboxamide [1506];
N-(1-methylpiperidin-4-yl)-5-(2-(3,3,3-trifluoropropyl)-7H-pyrrolo [2,3-d]pyrimidin-5-yl)pyrazolo [1,5-a]pyridine-3-carboxamide [1507];
5-(2-cyclopropyl-7H-pyrrolo [2,3-d]pyrimidin-5-yl)-N-(1-methylpiperidin-4-yl)pyrazolo [1,5-a] pyridine-3-carboxamide [1508];
5-(2-cyclobutyl-7H-pyrrolo [2,3-d]pyrimidin-5-yl)-N-(1-methylpiperidin-4-yl)pyrazolo [1,5-a] pyridine-3-carboxamide [1509];
5-(2-(cyclobutylmethyl)-7H-pyrrolo [2,3-d]pyrimidin-5-yl)-N-(1-methylpipe ridin-4-yl)pyrazolo [1,5-a]pyridine-3-carboxamide [1510];
5-(2-amino-7H-pyrrolo [2,3-d] pyrimidin-5-yl)-N-(1-methylpiperidin-4-yl)pyrazolo [1,5-a] pyridine-3-carboxamide [1511];
5-(2-(methylamino)-7H-pyrrolo [2,3-d] pyrimidin-5-yl)-N-(1-methylpiperidin-4-yl)pyrazolo [1,5-a] pyridine-3-carboxamide [1512];
5-(2-(ethylamino)-7H-pyrrolo [2,3-d]pyrimidin-5-yl)-N-(1-methylpiperidin-4-yl)pyrazolo [1,5-a] pyridine-3-carboxamide [1513];
5-(2-(i sop ropylamino)-7H-pyrrolo [2,3-d] pyrimidin-5-yl)-N-(1-methylpiperidin-4-yl)pyrazolo [1,5-a]pyridine-3-carboxamide [1514];
5-(2-(isobutylamino)-7H-pyrrolo [2,3-d]pyrimidin-5-yl)-N-(1-methylpiperidin-4-yl)pyrazolo [1,5-a] pyridine-3-carboxamide [1515];
N-(1-methylpiperidin-4-yl)-5-(2-(neopentylamino)-7H-pyrrolo [2,3-d]pyrimidin-5-yl)pyrazolo [1,5-a]pyridine-3-carboxamide [1516];
(S)-5-(2-(sec-butylamino)-7H-pyrrolo [2,3-d] pyrimidin-5-yl)-N-(1-methylpipe ridin-4-yl)pyrazolo [1,5-a]pyridine-3-carboxamide [1517];
5-(2-((2-fluoro-2-methylpropyl)amino)-7H-pyrrolo [2,3-d]pyrimidin-5-yl)-N-(1-methylpiperidin-4-yl)pyrazolo[1,5-a]pyridine-3-carboxamide [1518];
5-(2-((2,2-difluoropropyl)amino)-7H-pyrrolo [2,3-d] pyrimidin-5-yl)-N-(1-methylpiperidin-4-yl)pyrazolo [1,5-a]pyridine-3-carboxamide [1519];
N-(1-methylpiperidin-4-yl)-5-(2-((2,2,2-trifluoroethypamino)-7H-pyrrolo [2,3-d] pyrimidin-5-yl)pyrazolo [1,5-a]pyridine-3-carboxamide [1520];
(R)-N-(1-methylpiperidin-4-yl)-5-(2-((1,1,1-trifluoropropan-2-yl)amino)-7H-pyrrolo [2,3-d]pyrimidin-5-yl)pyrazolo [1,5-a]pyridine-3-carboxamide [1521];
(S)-N-(1-methylpiperidin-4-yl)-5-(2-((1,1,1-trifluoropropan-2-yl)amino)-7H-pyrrolo [2,3-d]pyrimidin-5-yl)pyrazolo [1,5-a]pyridine-3-carboxamide [1522];
N-(1-methylpiperidin-4-yl)-5-(2-((3,3,3-trifluoropropyl)amino)-7H-pyrrolo [2,3-d]pyrimidin-5-yl)pyrazolo [1,5-a]pyridine-3-carboxamide [1523];
(S)-5-(2-((l-methoxypropan-2-yl)amino)-7H-pyrrolo [2,3-d]pyrimidin-5-yl)-N-(1-methylpiperidin-4-yl)pyrazolo [1,5-a]pyridine-3-carboxamide [1524];
5-(2-ethoxy-7H-pyrrolo [2,3-d] pyrimidin-5-yl)-N-(1-methylpiperidin-4-yl)pyrazolo [1,5-a] pyridine-3-carboxamide [1525];
5-(2-(((1-fluorocyclopropyl)methyl)amino)-7H-pyrrolo [2,3-d]pyrimidin-5-yl)-N-(1-methylpiperidin-4-yl)pyrazolo [1,5-a]pyridine-3-carboxamide [1526];
(R)-5-(2-((l-cyclopropylethyl)amino)-7H-pyrrolo [2,3-d]pyrimidin-5-yl)-N-(1-methylpiperidin-4-yl)pyrazolo [1,5-a]pyridine-3-carboxamide [1527];
5-(2-(((l-methylcyclopropyl)methyl)amino)-7H-pyrrolo [2,3-d]pyrimidin-5-yl)-N-(1-methylpiperidin-4-yl)pyrazolo [1,5-a]pyridine-3-carboxamide [1528];
N-(1-me thylpiperidin-4-yl)-5-(2-(41-(trifluorome thyl)cyclopropyl)methyl)amino)-7H-pyrrolo [2,3-d]pyrimidin-5-yl)pyrazolo [1,5-a]pyridine-3-carboxamide [1529];
5-(2-((2-cyclopropyl-2,2-difluoroethypamino)-7H-pyrrolo [2,3-d]pyrimidin-5-yl)-N-(1-methylpiperidin-4-yl)pyrazolo [1,5-a]pyridine-3-carboxamide [1530];
5-(2-((3,3-difluorocyclobutyl)amino)-7H-pyrrolo [2,3-d]pyrimidin-5-yl)-N-(1-methylpipe ridin-4-yl)pyrazolo [1,5-a]pyridine-3-carboxamide [1531];
5-(2-(((1-fluorocyclobutyl)methyl)amino)-7H-pyrrolo [2,3-d]pyrimidin-5-yl)-N-(1-methylpiperidin-4-yl)pyrazolo [1,5-a]pyridine-3-carboxamide [1532];
5-(2-((4,4-difluorocyclohexyl)amino)-7H-pyrrolo [2,3-d]pyrimidin-5-yl)-N-(1-methylpiperidin-4-yl)pyrazolo [1,5-a]pyridine-3-carboxamide [1533];
5-(2-((cis-4-methoxycyclohexyl)amino)-7H-pyrrolo [2,3-d]pyrimidin-5-yl)-N-(1-methylpipe ridin-4-yl)pyrazolo [1,5-a]pyridine-3-carboxamide [1534];
5-(2-(((1-fluorocyclohexyl)me thyl)amino)-7H-pyrrolo [2,3-d]pyrimidin-5-yl)-N-(1-methylpiperidin-4-yl)pyrazolo [1,5-a]pyridine-3-carboxamide [1535];
5-(2-(((4,4-difluorocyclohexyl)me thyl)amino)-7H-pyrrolo [2,3-d]pyrimidin-5-yl)-N-(1-methylpiperidin-4-yl)pyrazolo [1,5-a]pyridine-3-carboxamide [1536];
N-(1-methylpiperidin-4-yl)-5-(2-((1-methylpiperidin-4-yl)amino)-7H-pyrrolo [2,3-d] pyrimidin-5-yl)pyrazolo [1,5-a]pyridine-3-carboxamide [1537];
N-(1-methylpiperidin-4-yl)-5-(2-(((1-methylpiperidin-4-yl)methyl)amino)-7H-pyrrolo [2,3-d] pyrimidin-5-yl)pyrazolo [1,5-a]pyridine-3-carboxamide [1538];
N-(1-methylpiperidin-4-yl)-5-(2-((tetrahydro-2H-pyran-4-yl)amino)-7H-pyrrolo [2,3-d] pyrimidin-5-yl)pyrazolo [1,5-a]pyridine-3-carboxamide [1539];
5-(2-((3-(4-methylpiperazin-1-yl)phenyl)amino)-7H-pyrrolo [2,3-d] pyrimidin-5-yl)-N-(1-methylpiperidin-4-yl)pyrazolo [1,5-a]pyridine-3-carboxamide [1540];
N-(1-methylpiperidin-4-yl)-5-(2-(pyridin-4-ylamino)-7H-pyrrolo [2,3-d] pyrimidin-5-yl)pyrazolo [1,5-a]pyridine-3-carboxamide [1541];
5-(2-((2-(4-methylpiperazin-1-yl)pyridin-4-yl)amino)-7H-pyrrolo [2,3-d]pyrimidin-5-yl)-N-(1-methylpiperidin-4-yl)pyrazolo [1,5-a]pyridine-3-carboxamide [1542];
5-(2-((6-(4-methylpiperazin-1-yl)pyridin-3-yl)amino)-7H-pyrrolo [2,3-d]pyrimidin-5-yl)-N-(1-methylpiperidin-4-yl)pyrazolo [1,5-a]pyridine-3-carboxamide [1543];
5-(2-(((6-(4-methylpiperazin-1-yl)pyridin-3-yl)methyl)amino)-7H-pyrrolo [2,3-d]pyrimidin-5-yl)-N-(1-methylpiperidin-4-yl)pyrazolo [1,5-a]pyridine-3-carboxamide [1544];
N-(1-methylpiperidin-4-yl)-5-(2-(quinolin-6-yl)-7H-pyrrolo [2,3-d]pyrimidin-5-yl)pyrazolo [1,5-a] pyridine-3-carboxamide [1545];
5-(4-(1-methyl-1H-pyrazol-4-yl)-7H-pyrrolo [2,3-d] pyrimidin-5-yl)-N-(1-methylpiperidin-4-yl)pyrazolo [1,5-a]pyridine-3-carboxamide [1546];
N-(1-methylpiperidin-4-yl)-5-(4-((trans-4-morpholinocyclohexyl)amino)-7H-pyrrolo [2,3-d]pyrimidin-5-yl)pyrazolo [1,5-a]pyridine-3-carboxamide [1547];
5-(2-isobutyl-7H-pyrrolo [2,3-d] pyrimidin-5-yl)-N-(tetrahydro-2H-pyran-4-yl)pyrazolo [1,5-a] pyridine-3-carboxamide [1549];
N-(tetrahydro-2H-pyran-4-yl)-5-(2-(3,3,3-trifluoropropyl)-7H-pyrrolo [2,3-d] pyrimidin-5-yl)pyrazolo [1,5-a]pyridine-3-carboxamide [1550];
5-(2-cyclopropyl-7H-pyrrolo [2,3-d]pyrimidin-5-yl)-N-(tetrahydro-2H-pyran-4-yl)pyrazolo [1,5-a] pyridine-3-carboxamide [1551];
5-(2-cyclopropyl-7H-pyrrolo [2,3-d]pyrimidin-5-yl)-N-methyl-N-(tetrahydro-2H-pyran-4-yl)pyrazolo [1,5-a]pyridine-3-carboxamide [1552];
5-(2-cyclobutyl-7H-pyrrolo [2,3-d]pyrimidin-5-yl)-N-(tetrahydro-2H-pyran-4-yl)pyrazolo [1,5-a] pyridine-3-carboxamide [1553];
5-(2-(cyclobutylmethyl)-7H-pyrrolo [2,3-d]pyrimidin-5-yl)-N-(tetrahydro-2H-pyran-4-yl)pyrazolo [1,5-a]pyridine-3-carboxamide [1554];
5-(2-amino-7H-pyrrolo [2,3-d] pyrimidin-5-yl)-N-(tetrahydro-2H-pyran-4-yl)pyrazolo [1,5-a] pyridine-3-carboxamide [1555];
5-(2-(methylamino)-7H-pyrrolo [2,3-d] pyrimidin-5-yl)-N-(tetrahydro-2H-pyran-4-yl)pyrazolo [1,5-a]pyridine-3-carboxamide [1556];
5-(2-(ethylamino)-7H-pyrrolo [2,3-d]pyrimidin-5-yl)-N-(tetrahydro-2H-pyran-4-yl)pyrazolo [1,5-a] pyridine-3-carboxamide [1557];
5-(2-(i sop ropylamino)-7H-pyrrolo [2,3-d] pyrimidin-5-yl)-N-(tetrahydro-2H-pyran-4-yl)pyrazolo [1,5-a]pyridine-3-carboxamide [1558];
5-(2-(isobutylamino)-7H-pyrrolo [2,3-d]pyrimidin-5-yl)-N-(tetrahydro -2H-pyran-4-yl)pyrazolo [1,5-a]pyridine-3-carboxamide [1559];
5-(2-(neopentylamino)-7H-pyrrolo [2,3-d]pyrimidin-5-yl)-N-(tetrahydro-2H-pyran-4-yl)pyrazolo [1,5-a]pyridine-3-carboxamide [1560];
(S)-5-(2-(sec-butylamino)-7H-pyrrolo [2,3-d] pyrimidin-5-yl)-N-(tetrahydro-2H-pyran-4-yl)pyrazolo [1,5-a]pyridine-3-carboxamide [1561];
5-(2-((2-fluoro-2-methylpropyl)amino)-7H-pyrrolo [2,3-d]pyrimidin-5-yl)-N-(tetrahydro-2H-pyran-4-yl)pyrazolo [1,5-a] pyridine-3-carboxamide [1562];
5-(2-((2,2-difluoropropyl)amino)-7H-pyrrolo [2,3-d] pyrimidin-5-yl)-N-(tetrahydro-2H-pyran-4-yl)pyrazolo [1,5-a]pyridine-3-carboxamide [1563];
N-(tetrahydro-2H-pyran-4-yl)-5-(2-((2,2,2-trifluoroethyl)amino)-7H-pyrrolo [2,3-d]pyrimidin-5-yl)pyrazolo [1,5-a]pyridine-3-carboxamide [1564];
(R)-N-(tetrahydro-2H-pyran-4-yl)-5-(2-((1,1,1-trifluoropropan-2-yl)amino)-7H-pyrrolo [2,3-d]pyrimidin-5-yl)pyrazolo [1,5-a]pyridine-3-carboxamide [1565];
(S)-N-(tetrahydro-2H-pyran-4-yl)-5-(2-((1,1,1-trifluoropropan-2-yl)amino)-7H-pyrrolo [2,3-d]pyrimidin-5-yl)pyrazolo [1,5-a]pyridine-3-carboxamide [1566];
N-(tetrahydro-2H-pyran-4-yl)-5-(2-((3,3,3-trifluoropropyl)amino)-7H-pyrrolo [2,3-d]pyrimidin-5-yl)pyrazolo [1,5-a]pyridine-3-carboxamide [1567];
(S)-5-(2-((l-methoxypropan-2-yl)amino)-7H-pyrrolo [2,3-d]pyrimidin-5-yl)-N-(tetrahydro-2H-pyran-4-yl)pyrazolo [1,5-a] pyridine-3-carboxamide [1568];
5-(2-ethoxy-7H-pyrrolo [2,3-d] pyrimidin-5-yl)-N-(tetrahydro -2H-pyran-4-yl)pyrazolo [1,5-a] pyridine-3-carboxamide [1569];
5-(2-(((1-fluorocyclopropyl)methyl)amino)-7H-pyrrolo [2,3-d]pyrimidin-5-yl)-N-(tetrahydro-2H-pyran-4-yl)pyrazolo [1,5-a]pyridine-3-carboxamide [1570];
(R)-5-(2-((l-cyclopropylethyl)amino)-7H-pyrrolo [2,3-d]pyrimidin-5-yl)-N-(tetrahydro-2H-pyran-4-yl)pyrazolo[1,5-a]pyridine-3-carboxamide [1571];
5-(2-(((l-me thylcyclopropyl)methyl)amino)-7H-pyrrolo [2,3-d]pyrimidin-5-yl)-N-(tetrahydro-2H-pyran-4-yl)pyrazolo [1,5-a]pyridine-3-carboxamide [1572];
N-(tetrahydro-2H-pyran-4-yl)-5-(2-(((1-(trifluoromethyl)cyclopropyl)methyl)amino)-7H-pyrrolo [2,3-d]pyrimidin-5-yl)pyrazolo [1,5-a]pyridine-3-carboxamide [1573];
5-(2-((2-cyclopropyl-2,2-difluoroethyl)amino)-7H-pyrrolo [2,3-d]pyrimidin-5-yl)-N-(tetrahydro-2H-pyran-4-yl)pyrazolo [1,5-a]pyridine-3-carboxamide [1574];
5-(2-((3,3-difluorocyclobutyl)amino)-7H-pyrrolo [2,3-d]pyrimidin-5-yl)-N-(tetrahydro-2H-pyran-4-yl)pyrazolo [1,5-a]pyridine-3-carboxamide [1575];
5-(2-(((1-fluorocyclobutyl)methyl)amino)-7H-pyrrolo [2,3-d]pyrimidin-5-yl)-N-(tetrahydro-2H-pyran-4-yl)pyrazolo [1,5-a]pyridine-3-carboxamide [1576];
5-(2-((4,4-difluorocyclohexyl)amino)-7H-pyrrolo [2,3-d]pyrimidin-5-yl)-N-(tetrahydro -2H-pyran-4-yl)pyrazolo [1,5-a]pyridine-3-carboxamide [1577];
5-(2-((cis-4-methoxycyclohexyl)amino)-7H-pyrrolo [2,3-d]pyrimidin-5-yl)-N-(tetrahydro-2H-pyran-4-yl)pyrazolo [1,5-a] pyridine-3-carboxamide [1578];
5-(2-(((1-fluorocyclohexyl)me thyl)amino)-7H-pyrrolo [2,3-d]pyrimidin-5-yl)-N-(tetrahydro-2H-pyran-4-yl)pyrazolo [1,5-a] pyridine-3-carboxamide [1579];
5-(2-4(4,4-difluorocyclohexyl)me thyl)amino)-7H-pyrrolo [2,3-d] pyrimidin-5-yl)-N-(tetrahydro-2H-pyran-4-yl)pyrazolo [1,5-a]pyridine-3-carboxamide [1580];
5-(2-((l-methylpipe ridin-4-yl)amino)-7H-pyrrolo [2,3-d] pyrimidin-5-yl)-N-(tetrahydro -2H-pyran-4-yl)pyrazolo [1,5-a]pyridine-3-carboxamide [1581];
5-(2-(((l-me thylpiperidin-4-yl)methyl)amino)-7H-pyrrolo [2,3-d] pyrimidin-5-yl)-N-(tetrahydro-2H-pyran-4-yl)pyrazolo [1,5-a]pyridine-3-carboxamide [1582];
N-(tetrahydro-2H-pyran-4-yl)-5-(2-((tetrahydro-2H-pyran-4-yl)amino)-7H-pyrrolo [2,3-d] pyrimidin-5-yl)pyrazolo [1,5-a]pyridine-3-carboxamide [1583];
5-(2-((3-(4-methylpiperazin-1-yl)phenyl)amino)-7H-pyrrolo [2,3-d] pyrimidin-5-yl)-N-(tetrahydro-2H-pyran-4-yl)pyrazolo [1,5-a] pyridine-3-carboxamide [1584];
5-(2-(pyridin-4-ylamino)-7H-pyrrolo [2,3-d] pyrimidin-5-yl)-N-(tetrahydro-2H-pyran-4-yl)pyrazolo [1,5-a]pyridine-3-carboxamide [1585];
5-(2-((2-(4-methylpiperazin-1-yl)pyridin-4-yl)amino)-7H-pyrrolo [2,3-d]pyrimidin-5-yl)-N-(tetrahydro-2H-pyran-4-yl)pyrazolo [1,5-a] pyridine-3-carboxamide [1586];
5-(2-((6-(4-methylpiperazin-1-yl)pyridin-3-yl)amino)-7H-pyrrolo [2,3-d]pyrimidin-5-yl)-N-(tetrahydro-2H-pyran-4-yl)pyrazolo [1,5-a] pyridine-3-carboxamide [1587];
5-(2-(((6-(4-methylpiperazin-1-yl)pyridin-3-yl)methyl)amino)-7H-pyrrolo [2,3-d]pyrimidin-5-yl)-N-(tetrahydro-2H-pyran-4-yl)pyrazolo [1,5-a]pyridine-3-carboxamide [1588];
5-(2-(quinolin-6-yl)-7H-pyrrolo [2,3-d] pyrimidin-5-yl)-N-(tetrahydro-2H-pyran-4-yl)pyrazolo [1,5-a]pyridine-3-carboxamide [1589];
5-(4-(1-methyl-1H-pyrazol-4-yl)-7H-pyrrolo [2,3-d] pyrimidin-5-yl)-N-(tetrahydro-2H-pyran-4-yl)pyrazolo [1,5-a]pyridine-3-carboxamide [1590];
5-(4-((trans-4-morpholinocyclohexyl)amino)-7H-pyrrolo [2,3-d] pyrimidin-5-yl)-N-(tetrahydro-2H-pyran-4-yl)pyrazolo [1,5-a]pyridine-3-carboxamide [1591];
5-(2-isobutyl-7H-pyrrolo [2,3-d] pyrimidin-5-yl)-N-(pyridin-3-yl)pyrazolo [1,5-a]pyridine-3-carboxamide [1593];
N-(pyridin-3-yl)-5-(2-(3,3,3-trifluoropropyl)-7H-pyrrolo [2,3-d]pyrimidin-5-yl)pyrazolo [1,5-a] pyridine-3-carboxamide [1594];
5-(2-cyclopropyl-7H-pyrrolo [2,3-d]pyrimidin-5-yl)-N-(pyridin-3-yl)pyrazolo [1,5-a]pyridine-3-carboxamide [1595];
5-(2-cyclobutyl-7H-pyrrolo [2,3-d]pyrimidin-5-yl)-N-(pyridin-3-yl)pyrazolo [1,5-a]pyridine-3-carboxamide [1596];
5-(2-(cyclobutylmethyl)-7H-pyrrolo [2,3-d]pyrimidin-5-yl)-N-(pyridin-3-yl)pyrazolo [1,5-a] pyridine-3-carboxamide [1597];
5-(2-amino-7H-pyrrolo [2,3-d] pyrimidin-5-yl)-N-(pyridin-3-yl)pyrazolo [1,5-a]pyridine-3-carboxamide [1598];
5-(2-(methylamino)-7H-pyrrolo [2,3-d] pyrimidin-5-yl)-N-(pyridin-3-yl)pyrazolo [1,5-a]pyridine-3-carboxamide [1599];
5-(2-(ethylamino)-7H-pyrrolo [2,3-d]pyrimidin-5-yl)-N-(pyridin-3-yl)pyrazolo [1,5-a]pyridine-3-carboxamide [1600];
5-(2-(i sop ropylamino)-7H-pyrrolo [2,3-d] pyrimidin-5-yl)-N-(pyridin-3-yl)pyrazolo [1,5-a] pyridine-3-carboxamide [1601];
5-(2-(isobutylamino)-7H-pyrrolo [2,3-d]pyrimidin-5-yl)-N-(pyridin-3-yl)pyrazolo [1,5-a] pyridine-3-carboxamide [1602];
5-(2-(neopentylamino)-7H-pyrrolo [2,3-d]pyrimidin-5-yl)-N-(pyridin-3-yl)pyrazolo [1,5-a] pyridine-3-carboxamide [1603];
(S)-5-(2-(sec-butylamino)-7H-pyrrolo [2,3-d] pyrimidin-5-yl)-N-(pyridin-3-yl)pyrazolo [1,5-a] pyridine-3-carboxamide [1604];
5-(2-((2-fluoro-2-methylpropyl)amino)-7H-pyrrolo [2,3-d]pyrimidin-5-yl)-N-(pyridin-3-yl)pyrazolo [1,5-a]pyridine-3-carboxamide [1605];
5-(2-((2,2-difluoropropyl)amino)-7H-pyrrolo [2,3-d] pyrimidin-5-yl)-N-(pyridin-3-yl)pyrazolo [1,5-a]pyridine-3-carboxamide [1606];
N-(pyridin-3-yl)-5-(2-((2,2,2-trifluoroethypamino)-7H-pyrrolo [2,3-d] pyrimidin-5-yl)pyrazolo [1,5-a]pyridine-3-carboxamide [1607];
(R)-N-(pyridin-3-yl)-5-(2-((1,1,1-trifluoropropan-2-yl)amino)-7H-pyrrolo [2,3-d]pyrimidin-5-yl)pyrazolo [1,5-a]pyridine-3-carboxamide [1608];
(S)-N-(pyridin-3-yl)-5-(2-((1,1,1-trifluoropropan-2-yl)amino)-7H-pyrrolo [2,3-d]pyrimidin-5-yl)pyrazolo [1,5-a]pyridine-3-carboxamide [1609];
N-(pyridin-3-yl)-5-(2-((3,3,3-trifluoropropyl)amino)-7H-pyrrolo [2,3-d] pyrimidin-5-yl)pyrazolo [1,5-a]pyridine-3-carboxamide [1610];
(S)-5-(2-((l-methoxypropan-2-yl)amino)-7H-pyrrolo [2,3-d]pyrimidin-5-yl)-N-(pyridin-3-yl)pyrazolo [1,5-a]pyridine-3-carboxamide [1611];
5-(2-ethoxy-7H-pyrrolo [2,3-d] pyrimidin-5-yl)-N-(pyridin-3-yl)pyrazolo [1,5-a]pyridine-3-carboxamide [1612];
5-(2-(((1-fluorocyclopropyl)methyl)amino)-7H-pyrrolo [2,3-d] pyrimidin-5-yl)-N-(pyridin-3-yl)pyrazolo [1,5-a]pyridine-3-carboxamide [1613];
(R)-5-(2-((l-cyclopropylethyl)amino)-7H-pyrrolo [2,3-d]pyrimidin-5-yl)-N-(pyridin-3-yl)pyrazolo [1,5-a]pyridine-3-carboxamide [1614];
5-(2-(((l-me thylcyclopropyl)me thyl)amino)-7H-pyrrolo [2,3-d] pyrimidin-5-yl)-N-(pyridin-3-yl)pyrazolo [1,5-a]pyridine-3-carboxamide [1615];
N-(pyridin-3-yl)-5-(2-(41-(trifluoromethyl)cyclopropyl)methyl)amino)-7H-pyrrolo [2,3-d]pyrimidin-5-yl)pyrazolo [1,5-a]pyridine-3-carboxamide [1616];
5-(2-((2-cyclopropyl-2,2-difluoroethypamino)-7H-pyrrolo [2,3-d]pyrimidin-5-yl)-N-(pyridin-3-yl)pyrazolo [1,5-a]pyridine-3-carboxamide [1617];
5-(2-((3,3-difluorocyclobutyl)amino)-7H-pyrrolo [2,3-d]pyrimidin-5-yl)-N-(pyridin-3-yl)pyrazolo [1,5-a]pyridine-3-carboxamide [1618];
5-(2-(((1-fluorocyclobutyl)methyl)amino)-7H-pyrrolo [2,3-d]pyrimidin-5-yl)-N-(pyridin-3-yl)pyrazolo [1,5-a]pyridine-3-carboxamide [1619];
5-(2-((4,4-difluorocyclohexyl)amino)-7H-pyrrolo [2,3-d]pyrimidin-5-yl)-N-(pyridin-3-yl)pyrazolo [1,5-a]pyridine-3-carboxamide [1620];
5-(2-((cis-4-methoxycyclohexyl)amino)-7H-pyrrolo [2,3-d]pyrimidin-5-yl)-N-(pyridin-3-yl)pyrazolo [1,5-a]pyridine-3-carboxamide [1621];
5-(2-(((1-fluorocyclohexyl)me thyl)amino)-7H-pyrrolo [2,3-d]pyrimidin-5-yl)-N-(pyridin-3-yl)pyrazolo [1,5-a]pyridine-3-carboxamide [1622];
5-(2-(((4,4-difluorocyclohexyl)me thyl)amino)-7H-pyrrolo [2,3-d] pyrimidin-5-yl)-N-(pyridin-3-yl)pyrazolo [1,5-a]pyridine-3-carboxamide [1623];
5-(2-((l-methylpipe ridin-4-yl)amino)-7H-pyrrolo [2,3-d] pyrimidin-5-yl)-N-(pyridin-3-yl)pyrazolo [1,5-a]pyridine-3-carboxamide [1624];
5-(2-(((1-me thylpiperidin-4-yl)methyl)amino)-7H-pyrrolo [2,3-d] pyrimidin-5-yl)-N-(pyridin-3-yl)pyrazolo [1,5-a]pyridine-3-carboxamide [1625];
N-(pyridin-3-yl)-5-(2-((tetrahydro -2H-pyran-4-yl)amino)-7H-pyrrolo [2,3-d]pyrimidin-5-yl)pyrazolo [1,5-a]pyridine-3-carboxamide [1626];
5-(2-((3-(4-methylpiperazin-1-yl)phenyl)amino)-7H-pyrrolo [2,3-d] pyrimidin-5-yl)-N-(pyridin-3-yl)pyrazolo [1,5-a]pyridine-3-carboxamide [1627];
N-(pyridin-3-yl)-5-(2-(pyridin-4-ylamino)-7H-pyrrolo [2,3-d]pyrimidin-5-yl)pyrazolo [1,5-a] pyridine-3-carboxamide [1628];
5-(2-((2-(4-methylpiperazin-1-yl)pyridin-4-yl)amino)-7H-pyrrolo [2,3-d]pyrimidin-5-yl)-N-(pyridin-3-yl)pyrazolo [1,5-a]pyridine-3-carboxamide [1629];
5-(2-((6-(4-methylpiperazin-1-yl)pyridin-3-yl)amino)-7H-pyrrolo [2,3-d]pyrimidin-5-yl)-N-(pyridin-3-yl)pyrazolo [1,5-a]pyridine-3-carboxamide [1630];
5-(2-(((6-(4-methylpiperazin-1-yl)pyridin-3-yl)methyl)amino)-7H-pyrrolo [2,3-d]pyrimidin-5-yl)-N-(pyridin-3-yl)pyrazolo [1,5-a]pyridine-3-carboxamide [1631];
N-(pyridin-3-yl)-5-(2-(quinolin-6-yl)-7H-pyrrolo [2,3-d]pyrimidin-5-yl)pyrazolo [1,5-a]pyridine-3-carboxamide [1632];
5-(4-(1-methyl-1H-pyrazol-4-yl)-7H-pyrrolo [2,3-d] pyrimidin-5-yl)-N-(pyridin-3-yl)pyrazolo [1,5-a] pyridine-3-carboxamide [1633];
5-(4-((trans-4-morpholinocyclohexyl)amino)-7H-pyrrolo [2,3-d] pyrimidin-5-yl)-N-(pyridin-3-yl)pyrazolo [1,5-a]pyridine-3-carboxamide [1634];
2-i sobutyl-5-(pyrazolo [1,5-a]pyrimidin-5-yl)-7H-pyrrolo [2,3-d]pyrimidine [1636];
5-(pyrazolo [1,5-a]pyrimidin-5-yl)-2-(3,3,3-trifluoropropyl)-7H-pyrrolo [2,3-d]pyrimidine [1637];
2-cyclopropyl-5-(pyrazolo [1,5-a] pyrimidin-5-yl)-7H-pyrrolo [2,3-d] pyrimidine [1638];
2-cyclobutyl-5-(pyrazolo [1,5-a]pyrimidin-5-yl)-7H-pyrrolo [2,3-d]pyrimidine [1639];
2-(cyclobutylmethyl)-5-(pyrazolo [1,5-a]pyrimidin-5-yl)-7H-pyrrolo [2,3-d]pyrimidine [1640];
5-(pyrazolo [1,5-a]pyrimidin-5-yl)-7H-pyrrolo [2,3-d]pyrimidin-2-amine [1641];
N-methyl-5-(pyrazolo [1,5-a]pyrimidin-5-yl)-7H-pyrrolo [2,3-d]pyrimidin-2-amine [1642];
N-ethyl-5-(pyrazolo [1,5-a] pyrimidin-5-yl)-7H-pyrrolo [2,3-d] pyrimidin-2-amine [1643];
N-isopropyl-5-(pyrazolo [1,5-a]pyrimidin-5-yl)-7H-pyrrolo [2,3-d]pyrimidin-2-amine [1644];
N-isobutyl-5-(pyrazolo [1,5-a] pyrimidin-5-yl)-7H-pyrrolo [2,3-d] pyrimidin-2-amine [1645];
N-neopentyl-5-(pyrazolo [1,5-a] pyrimidin-5-yl)-7H-pyrrolo [2,3-d]pyrimidin-2-amine [1646];
(S)-N-(sec-butyl)-5-(pyrazolo [1,5-a]pyrimidin-5-yl)-7H-pyrrolo [2,3-d]pyrimidin-2-amine [1647];
N-(2-fluoro-2-methylpropyl)-5-(pyrazolo [1,5-a]pyrimidin-5-yl)-7H-pyrrolo [2,3-d]pyrimidin-2-amine [1648];
N-(2,2-difluoropropyl)-5-(pyrazolo [1,5-a]pyrimidin-5-yl)-7H-pyrrolo [2,3-d]pyrimidin-2-amine [1649];
5-(pyrazolo [1,5-a]pyrimidin-5-yl)-N-(2,2,2-trifluoroethyl)-7H-pyrrolo [2,3-d] pyrimidin-2-amine [1650];
(R)-5-(pyrazolo [1,5-a]pyrimidin-5-yl)-N-(1,1,1-trifluoropropan-2-yl)-7H-pyrrolo [2,3-d]pyrimidin-2-amine [1651];
(S)-5-(pyrazolo [1,5-a]pyrimidin-5-yl)-N-(1,1,1-trifluoropropan-2-yl)-7H-pyrrolo [2,3-d]pyrimidin-2-amine [1652];
5-(pyrazolo [1,5-a]pyrimidin-5-yl)-N-(3 ,3,3-trifluoropropyl)-7H-pyrrolo [2,3-d] pyrimidin-2-amine [1653];
(S)-N-(1-methoxypropan-2-yl)-5-(pyrazolo [1,5-a]pyrimidin-5-yl)-7H-pyrrolo [2,3-d]pyrimidin-2-amine [1654];
2-ethoxy-5-(pyrazolo [1,5-a]pyrimidin-5-yl)-7H-pyrrolo [2,3-d]pyrimidine [1655];
N-((1-fluorocyclopropyl)methyl)-5-(pyrazolo [1,5-a] pyrimidin-5-yl)-7H-pyrrolo [2,3-d]pyrimidin-2-amine [1656];
(R)-N-(1-cyclopropylethyl)-5-(pyrazolo [1,5-a] pyrimidin-5-yl)-7H-pyrrolo [2,3-d]pyrimidin-2-amine [1657];
N-((1-methylcyclopropyl)methyl)-5-(pyrazolo [1,5-a] pyrimidin-5-yl)-7H-pyrrolo [2,3-d]pyrimidin-2-amine [1658];
5-(pyrazolo [1,5-a]pyrimidin-5-yl)-N-4 1-(trifluoromethyl)cyclopropyl)methyl)-7H-pyrrolo [2,3-d]pyrimidin-2-amine [1659];
N-(2-cyclopropyl-2,2-difluoroethyl)-5-(pyrazolo [1,5-a] pyrimidin-5-yl)-7H-pyrrolo [2,3-d]pyrimidin-2-amine [1660];
N-(3,3-difluorocyclobutyl)-5-(pyrazolo [1,5-a]pyrimidin-5-yl)-7H-pyrrolo [2,3-d]pyrimidin-2-amine [1661];
N-((1-fluorocyclobutypmethyl)-5-(pyrazolo [1,5-a]pyrimidin-5-yl)-7H-pyrrolo [2,3-d]pyrimidin-2-amine [1662];
N-(4,4-difluorocyclohexyl)-5-(pyrazolo [1,5-a]pyrimidin-5-yl)-7H-pyrrolo [2,3-d] pyrimidin-2-amine [1663];
N-(cis-4-methoxycyclohexyl)-5-(pyrazolo [1,5-a]pyrimidin-5-yl)-7H-pyrrolo [2,3-d]pyrimidin-2-amine [1664];
N-((1-fluorocyclohexyl)me thyl)-5-(pyrazolo [1,5-a]pyrimidin-5-yl)-7H-pyrrolo [2,3-d]pyrimidin-2-amine [1665];
N-((4,4-difluorocyclohexyl)methyl)-5-(pyrazolo [1,5-a] pyrimidin-5-yl)-7H-pyrrolo [2,3-d]pyrimidin-2-amine [1666];
N-(1-methylpiperidin-4-yl)-5-(pyrazolo [1,5-a] pyrimidin-5-yl)-7H-pyrrolo [2,3-d]pyrimidin-2-amine [1667];
N-((1-methylpiperidin-4-yl)methyl)-5-(pyrazolo [1,5-a]pyrimidin-5-yl)-7H-pyrrolo [2,3-d]pyrimidin-2-amine [1668];
5-(pyrazolo [1,5-a]pyrimidin-5-yl)-N-(tetrahydro-2H-pyran-4-yl)-7H-pyrrolo [2,3-d]pyrimidin-2-amine [1669];
N-(3-(4-methylpiperazin-1-yl)phenyl)-5-(pyrazolo [1,5-a]pyrimidin-5-yl)-7H-pyrrolo [2,3-d]pyrimidin-2-amine [1670];
5-(pyrazolo [1,5-a]pyrimidin-5-yl)-N-(pyridin-4-yl)-7H-pyrrolo [2,3-d]pyrimidin-2-amine [1671];
N-(2-(4-methylpiperazin-1-yl)pyridin-4-yl)-5-(pyrazolo [1,5-a]pyrimidin-5-yl)-7H-pyrrolo [2,3-d]pyrimidin-2-amine [1672];
N-(6-(4-methylpiperazin-1-yl)pyridin-3-yl)-5-(pyrazolo [1,5-a]pyrimidin-5-yl)-7H-pyrrolo [2,3-d]pyrimidin-2-amine [1673];
N-((6-(4-methylpiperazin-1-yl)pyridin-3-yl)methyl)-5-(pyrazolo [1,5-a] pyrimidin-5-yl)-7H-pyrrolo [2,3-d]pyrimidin-2-amine [1674];
6-(5-(pyrazolo [1,5-a]pyrimidin-5-yl)-7H-pyrrolo [2,3-d]pyrimidin-2-yl)quinoline [1675];
4-(1-methyl-1H-pyrazol-4-yl)-5-(pyrazolo [1,5-a]pyrimidin-5-yl)-7H-pyrrolo [2,3-d] pyrimidine [1676];
N-(trans-4-morpholinocyclohexyl)-5-(pyrazolo [1,5-a]pyrimidin-5-yl)-7H-pyrrolo [2,3-d]pyrimidin-4-amine [1677];
N-(2-fluoro-2-methylpropyl)-5-(3-fluoropyrazolo [1,5-a]pyrimidin-5-yl)-7H-pyrrolo [2,3-d]pyrimidin-2-amine [1678];
(R)-5-(3-fluoropyrazolo [1,5-a]pyrimidin-5-yl)-N-(1,1,1-trifluoropropan-2-yl)-7H-pyrrolo [2,3-d]pyrimidin-2-amine [1679];
5-(3-chloropyrazolo [1,5-a] pyrimidin-5-yl)-N-(2-fluoro-2-methylpropyl)-7H-pyrrolo [2,3-d]pyrimidin-2-amine [1680];
(R)-5-(3-chloropyrazolo [1,5-a]pyrimidin-5-yl)-N-(1,1,1-trifluoropropan-2-yl)-7H-pyrrolo [2,3-d]pyrimidin-2-amine [1681];
5-(3-cyclopropylpyrazolo [1,5-a] pyrimidin-5-yl)-24 sobutyl-7H-pyrrolo [2,3-d]pyrimidine [1683];
5-(3-cyclopropylpyrazolo [1,5-a] pyrimidin-5-yl)-2-(3,3,3-trifluoropropyl)-7H-pyrrolo [2,3-d]pyrimidine [1684];
2-cyclopropyl-5-(3-cyclopropylpyrazolo [1,5-a]pyrimidin-5-yl)-7H-pyrrolo [2,3-d]pyrimidine [1685];
2-cyclobutyl-5-(3-cyclopropylpyrazolo [1,5-a] pyrimidin-5-yl)-7H-pyrrolo [2,3-d]pyrimidine [1686];
2-(cyclobutylmethyl)-5-(3-cyclopropylpyrazolo [1,5-a]pyrimidin-5-yl)-7H-pyrrolo [2,3-d]pyrimidine [1687];
5-(3-cyclopropylpyrazolo [1,5-a] pyrimidin-5-yl)-7H-pyrrolo [2,3-d]pyrimidin-2-amine [1688];
5-(3-cyclopropylpyrazolo [1,5-a] pyrimidin-5-yl)-N-methyl-7H-pyrrolo [2,3-d]pyrimidin-2-amine [1689];
5-(3-cyclopropylpyrazolo [1,5-a] pyrimidin-5-yl)-N-ethyl-7H-pyrrolo [2,3-d]pyrimidin-2-amine [1690];
5-(3-cyclopropylpyrazolo [1,5-a] pyrimidin-5-yl)-N-isopropyl-7H-pyrrolo [2,3-d]pyrimidin-2-amine [1691];
5-(3-cyclopropylpyrazolo [1,5-a] pyrimidin-5-yl)-N-isobutyl-7H-pyrrolo [2,3-d] pyrimidin-2-amine ;
5-(3-cyclopropylpyrazolo [1,5-a] pyrimidin-5-yl)-N-neopentyl-7H-pyrrolo [2,3-d]pyrimidin-2-amine [1693];
(S)-N-(sec-butyl)-5-(3-cyclopropylpyrazolo [1,5-a] pyrimidin-5-yl)-7H-pyrrolo [2,3-d]pyrimidin-2-amine [1694];
5-(3-cyclopropylpyrazolo [1,5-a] pyrimidin-5-yl)-N-(2-fluoro-2-methylpropyl)-7H-pyrrolo [2,3-d]pyrimidin-2-amine [1695];
5-(3-cyclopropylpyrazolo [1,5-a] pyrimidin-5-yl)-N-(2,2-difluoropropyl)-7H-pyrrolo [2,3-d]pyrimidin-2-amine [1696];
5-(3-cyclopropylpyrazolo [1,5-a] pyrimidin-5-yl)-N-(2,2,2-trifluoroethyl)-7H-pyrrolo [2,3-d]pyrimidin-2-amine [1697];
(R)-5-(3-cyclopropylpyrazolo [1,5-a] pyrimidin-5-yl)-N-(1, 1, 1-trifluoropropan-2-yl)-7H-pyrrolo [2,3-d]pyrimidin-2-amine [1698];
(S)-5-(3-cyclopropylpyrazolo [1,5-a] pyrimidin-5-yl)-N-(1, 1, 1-trifluoropropan-2-yl)-7H-pyrrolo [2,3-d]pyrimidin-2-amine [1699];
5-(3-cyclopropylpyrazolo [1,5-a] pyrimidin-5-yl)-N-(3 ,3 ,3-trifluoropropyl)-7H-pyrrolo [2,3-d]pyrimidin-2-amine [1700];
(S)-5-(3-cyclopropylpyrazolo [1,5-a] pyrimidin-5-yl)-N-(1-methoxypropan-2-yl)-7H-pyrrolo [2,3-d]pyrimidin-2-amine [1701];
5-(3-cyclopropylpyrazolo [1,5-a] pyrimidin-5-yl)-2-ethoxy-7H-pyrrolo [2,3-d]pyrimidine [1702];
5-(3-cyclopropylpyrazolo [1,5-a] pyrimidin-5-yl)-N-((1-fluorocyclopropyl)methyl)-7H-pyrrolo [2,3-d]pyrimidin-2-amine [1703];
(R)-N-(1-cyclopropylethyl)-5-(3-cyclopropylpyrazolo [1,5-a] pyrimidin-5-yl)-7H-pyrrolo [2,3-d]pyrimidin-2-amine [1704];
5-(3-cyclopropylpyrazolo [1,5-a] pyrimidin-5-yl)-N-((1-methylcyclopropyl)methyl)-7H-pyrrolo [2,3-d]pyrimidin-2-amine [1705];
5-(3-cyclopropylpyrazolo [1,5-a] pyrimidin-5-yl)-N-4 1-(trifluoromethyl)cyclopropyl)methyl)-7H-pyrrolo [2,3-d]pyrimidin-2-amine [1706];
N-(2-cyclopropyl-2,2-difluoroethyl)-5-(3-cyclopropylpyrazolo [1,5-a] pyrimidin-5-yl)-7H-pyrrolo [2,3-d]pyrimidin-2-amine [1707];
5-(3-cyclopropylpyrazolo [1,5-a] pyrimidin-5-yl)-N-(3,3-difluorocyclobutyl)-7H-pyrrolo [2,3-d] pyrimidin-2-amine [1708];
5-(3-cyclopropylpyrazolo [1,5-a] pyrimidin-5-yl)-N-((1-fluorocyclobutyl)methyl)-7H-pyrrolo [2,3-d] pyrimidin-2-amine [1709];
5-(3-cyclopropylpyrazolo [1,5-a] pyrimidin-5-yl)-N-(4,4-difluorocyclohexyl)-7H-pyrrolo [2,3-d]pyrimidin-2-amine [1710];
5-(3-cyclopropylpyrazolo [1,5-a] pyrimidin-5-yl)-N-(cis-4-methoxycyclohexyl)-7H-pyrrolo [2,3-d]pyrimidin-2-amine [1711];
5-(3-cyclopropylpyrazolo [1,5-a] pyrimidin-5-yl)-N-((1-fluorocyclohexyl)methyl)-7H-pyrrolo [2,3-d]pyrimidin-2-amine [1712];
5-(3-cyclopropylpyrazolo [1,5-a] pyrimidin-5-yl)-N-((4,4-difluorocyclohexyl)methyl)-7H-pyrrolo [2,3-d]pyrimidin-2-amine [1713];
5-(3-cyclopropylpyrazolo [1,5-a] pyrimidin-5-yl)-N-(1-methylpipe ridin-4-yl)-7H-pyrrolo [2,3-d]pyrimidin-2-amine [1714];
5-(3-cyclopropylpyrazolo [1,5-a] pyrimidin-5-yl)-N-((1-methylpiperidin-4-yl)methyl)-7H-pyrrolo [2,3-d]pyrimidin-2-amine [1715];
5-(3-cyclopropylpyrazolo [1,5-a] pyrimidin-5-yl)-N-(tetrahydro-2H-pyran-4-yl)-7H-pyrrolo [2,3-d]pyrimidin-2-amine [1716];
5-(3-cyclopropylpyrazolo [1,5-a] pyrimidin-5-yl)-N-(3-(4-methylpiperazin-1-yl)phenyl)-7H-pyrrolo [2,3-d]pyrimidin-2-amine [1717];
5-(3-cyclopropylpyrazolo [1,5-a] pyrimidin-5-yl)-N-(pyridin-4-yl)-7H-pyrrolo [2,3-d] pyrimidin-2-amine [1718];
5-(3-cyclopropylpyrazolo [1,5-a] pyrimidin-5-yl)-N-(2-(4-methylpiperazin-1-yl)pyridin-4-yl)-7H-pyrrolo [2,3-d]pyrimidin-2-amine [1719];
5-(3-cyclopropylpyrazolo [1,5-a] pyrimidin-5-yl)-N-(6-(4-methylpiperazin-1-yl)pyridin-3-yl)-7H-pyrrolo [2,3-d]pyrimidin-2-amine [1720];
5-(3-cyclopropylpyrazolo [1,5-a] pyrimidin-5-yl)-N-((6-(4-methylpipe razin-1-yl)pyridin-3-yl)methyl)-7H-pyrrolo [2,3-d]pyrimidin-2-amine [1721];
6-(5-(3-cyclopropylpyrazolo [1,5-a]pyrimidin-5-yl)-7H-pyrrolo [2,3-d]pyrimidin-2-yl)quinoline [1722];
5-(3-cyclopropylpyrazolo [1,5-a] pyrimidin-5-yl)-4-(1-methyl-1H-pyrazol-4-yl)-7H-pyrrolo [2,3-d]pyrimidine [1723];
5-(3-cyclopropylpyrazolo[1,5-a]pyrimidin-5-yl)-N-(trans-4-morpholinocyclohexyl)-7H-pyrrolo[2,3-d]pyrimidin-4-amine [1724];
8-(2-isobutyl-7H-pyrrolo[2,3-d]pyrimidin-5-yl)-3,4-dihydrobenzo [1,4]oxazepin-5(2H)-one [1726];
8-(2-(3,3,3-trifluoropropyl)-7H-pyrrolo[2,3-d]pyrimidin-5-yl)-3,4-dihydrobenzo [1,4]oxazepin-5(2H)-one [1727];
8-(2-cyclopropyl-7H-pyrrolo[2,3-d]pyrimidin-5-yl)-3,4-dihydrobenzo[f][1,4]oxazepin-5(2H)-one [1728];
8-(2-cyclobutyl-7H-pyrrolo[2,3-d]pyrimidin-5-yl)-3,4-dihydrobenzo [1,4]oxazepin-5(2H)-one [1729];
8-(2-(cyclobutylmethyl)-7H-pyrrolo[2,3-d]pyrimidin-5-yl)-3,4-dihydrobenzo[f][1,4]oxazepin-5(2H)-one [1730];
8-(2-amino-7H-pyrrolo[2,3-d]pyrimidin-5-yl)-3,4-dihydrobenzo[f][1,4]oxazepin-5(2H)-one [1731];
8-(2-(methylamino)-7H-pyrrolo[2,3-d]pyrimidin-5-yl)-3,4-dihydrobenzo[f][1,4]oxazepin-5(2H)-one [1732];
8-(2-(ethylamino)-7H-pyrrolo[2,3-d]pyrimidin-5-yl)-3,4-dihydrobenzo [1,4]oxazepin-5(2H)-one [1733];
8-(2-(isopropylamino)-7H-pyrrolo[2,3-d]pyrimidin-5-yl)-3,4-dihydrobenzo[f][1,4]oxazepin-5(2H)-one [1734];
8-(2-(isobutylamino)-7H-pyrrolo[2,3-d]pyrimidin-5-yl)-3,4-dihydrobenzo [1,4]oxazepin-5(2H)-one [1735];
8-(2-(neopentylamino)-7H-pyrrolo[2,3-d]pyrimidin-5-yl)-3,4-dihydrobenzo [1,4]oxazepin-5(2H)-one [1736];
(S)-8-(2-(sec-butylamino)-7H-pyrrolo[2,3-d]pyrimidin-5-yl)-3,4-dihydrobenzo[f][1,4]oxazepin-5(2H)-one [1737];
8-(2-((2-fluoro-2-methylpropyl)amino)-7H-pyrrolo[2,3-d]pyrimidin-5-yl)-3,4-dihydrobenzo [1,4]oxazepin-5(2H)-one [1738];
8-(2-((2,2-difluoropropyl)amino)-7H-pyrrolo[2,3-d]pyrimidin-5-yl)-3,4-dihydrobenzo [1,4]oxazepin-5(2H)-one [1739];
8-(2-((2,2,2-trifluoroethyl)amino)-7H-pyrrolo[2,3-d]pyrimidin-5-yl)-3,4-dihydrobenzo [1,4]oxazepin-5(2H)-one [1740];
(R)-8-(2-((1,1,1-trifluoropropan-2-yl)amino)-7H-pyrrolo[2,3-d]pyrimidin-5-yl)-3,4-dihydrobenzo [1,4]oxazepin-5(2H)-one [1741];
(S)-8-(2-((1,1,1-trifluoropropan-2-yl)amino)-7H-pyrrolo [2,3-d]pyrimidin-5-yl)-3,4-dihydrobenzo [f] [1,4] oxazepin-5(2H)-one [1742];
8-(2-((3,3,3-trifluoropropyl)amino)-7H-pyrrolo [2,3-d]pyrimidin-5-yl)-3,4-dihydrobenzo [f] [1,4] oxazepin-5(2H)-one [1743];
(S)-8-(2-((l-methoxypropan-2-yl)amino)-7H-pyrrolo [2,3-d]pyrimidin-5-yl)-3,4-dihydrobenzo [f] [1,4] oxazepin-5(2H)-one [1744];
8-(2-ethoxy-7H-pyrrolo [2,3-d] pyrimidin-5-yl)-3,4-dihydrobenzo [f] [1,4] oxazepin-5(2H)-one [1745];
8-(2-(((1-fluorocyclopropyl)methyl)amino)-7H-pyrrolo [2,3-d]pyrimidin-5-yl)-3,4-dihydrobenzo [f] [1,4] oxazepin-5(2H)-one [1746];
(R)-8-(2-((l-cyclopropylethyl)amino)-7H-pyrrolo [2,3-d]pyrimidin-5-yl)-3,4-dihydrobenzo [f] [1,4] oxazepin-5(2H)-one [1747];
8-(2-(((l-methylcyclopropyl)methyl)amino)-7H-pyrrolo [2,3-d]pyrimidin-5-yl)-3,4-dihydrobenzo [f] [1,4] oxazepin-5(2H)-one [1748];
8-(2-(((1-(trifluoromethyl)cyclopropyl)me thyl)amino)-7H-pyrrolo [2,3-d] pyrimidin-5-yl)-3,4-dihydrobenzo [f] [1,4] oxazepin-5(2H)-one [1749];
8-(2-((2-cyclopropyl-2,2-difluoroethyl)amino)-7H-pyrrolo [2,3-d]pyrimidin-5-yl)-3,4-dihydrobenzo [f] [1,4] oxazepin-5(2H)-one [1750];
8-(2-((3,3-difluorocyclobutyl)amino)-7H-pyrrolo [2,3-d]pyrimidin-5-yl)-3,4-dihydrobenzo [f] [1,4] oxazepin-5(2H)-one [1751];
8-(2-(((1-fluorocyclobutyl)methyl)amino)-7H-pyrrolo [2,3-d]pyrimidin-5-yl)-3,4-dihydrobenzo [f] [1,4] oxazepin-5(2H)-one [1752];
8-(2-((4,4-difluorocyclohexyl)amino)-7H-pyrrolo [2,3-d]pyrimidin-5-yl)-3,4-dihydrobenzo [f] [1,4] oxazepin-5(2H)-one [1753];
8-(2-((cis-4-methoxycyclohexyl)amino)-7H-pyrrolo [2,3-d]pyrimidin-5-yl)-3,4-dihydrobenzo [f] [1,4] oxazepin-5(2H)-one [1754];
8-(2-(((1-fluorocyclohexyl)me thyl)amino)-7H-pyrrolo [2,3-d]pyrimidin-5-yl)-3,4-dihydrobenzo [f] [1,4] oxazepin-5(2H)-one [1755];
8-(2-(((4,4-difluorocyclohexyl)me thyl)amino)-7H-pyrrolo [2,3-d]pyrimidin-5-yl)-3,4-dihydrobenzo [f] [1,4] oxazepin-5(2H)-one [1756];
8-(2-((l-methylpipe ridin-4-yl)amino)-7H-pyrrolo [2,3-d]pyrimidin-5-yl)-3,4-dihydrobenzo [f] [1,4] oxazepin-5(2H)-one [1757];
8-(2-(((l-methylpiperidin-4-yl)methyl)amino)-7H-pyrrolo [2,3-d] pyrimidin-5-yl)-3,4-dihydrobenzo [f] [1,4] oxazepin-5(2H)-one [1758];
8-(2-((tetrahydro-2H-pyran-4-yl)amino)-7H-pyrrolo [2,3-d]pyrimidin-5-yl)-3,4-dihydrobenzo [f] [1,4] oxazepin-5(2H)-one [1759];
8-(2-((3-(4-methylpiperazin-1-yl)phenyl)amino)-7H-pyrrolo [2,3-d] pyrimidin-5-yl)-3,4-dihydrobenzo [f] [1,4] oxazepin-5(2H)-one [1760];
8-(2-(pyridin-4-ylamino)-7H-pyrrolo [2,3-d] pyrimidin-5-yl)-3,4-dihydrobenzo [f] [1,4] oxazepin-5(2H)-one [1761];
8-(2-((2-(4-methylpiperazin-1-yl)pyridin-4-yl)amino)-7H-pyrrolo [2,3-d]pyrimidin-5-yl)-3,4-dihydrobenzo [f] [1,4] oxazepin-5(2H)-one [1762];
8-(2-((6-(4-methylpiperazin-1-yl)pyridin-3-yl)amino)-7H-pyrrolo [2,3-d]pyrimidin-5-yl)-3,4-dihydrobenzo [f] [1,4] oxazepin-5(2H)-one [1763];
8-(2-(((6-(4-methylpiperazin-1-yl)pyridin-3-yl)methyl)amino)-7H-pyrrolo [2,3-d]pyrimidin-5-yl)-3,4-dihydrobenzo [f] [1,4] oxazepin-5(2H)-one [1764];
8-(2-(quinolin-6-yl)-7H-pyrrolo [2,3-d] pyrimidin-5-yl)-3,4-dihydrobenzo [f][1,4] oxazepin-5(2H)-one [1765];
8-(4-(1-methyl-1H-pyrazol-4-yl)-7H-pyrrolo [2,3-d] pyrimidin-5-yl)-3,4-dihydrobenzo [f] [1,4] oxazepin-5(2H)-one [1766];
8-(4-((trans-4-morpholinocyclohexyl)amino)-7H-pyrrolo [2,3-d] pyrimidin-5-yl)-3,4-dihydrobenzo [f] [1,4] oxazepin-5(2H)-one [1767];
5-(chroman-6-yl)-N-(2-(4-me thylpiperazin-1-yl)pyridin-4-yl)-7H-pyrrolo [2,3-d] pyrimidin-2-amine [1768];
7-(2-isobutyl-7H-pyrrolo [2,3-d] pyrimidin-5-yl)-2,2-dimethylchroman-4-one [1770];
2,2-dimethyl-7-(2-(3,3,3-trifluoropropyl)-7H-pyrrolo [2,3-d]pyrimidin-5-yl)chroman-4-one [1771];
7-(2-cyclopropyl-7H-pyrrolo [2,3-d]pyrimidin-5-yl)-2,2-dimethylchroman-4-one [1772];
7-(2-cyclobutyl-7H-pyrrolo [2,3-d]pyrimidin-5-yl)-2,2-dimethylchroman-4-one [1773];
7-(2-(cyclobutylmethyl)-7H-pyrrolo [2,3-d]pyrimidin-5-yl)-2,2-dimethylchroman-4-one [1774];
7-(2-amino-7H-pyrrolo [2,3-d] pyrimidin-5-yl)-2,2-dimethylchroman-4-one [1775];
2,2-dimethyl-7-(2-(methylamino)-7H-pyrrolo [2,3-d]pyrimidin-5-yl)chroman-4-one [1776];
7-(2-(ethylamino)-7H-pyrrolo [2,3-d]pyrimidin-5-yl)-2,2-dimethylchroman-4-one [1777];
7-(2-(i sop ropylamino)-7H-pyrrolo [2,3-d] pyrimidin-5-yl)-2,2-dimethylchroman-4-one [1778];
7-(2-(isobutylamino)-7H-pyrrolo [2,3-d]pyrimidin-5-yl)-2,2-dimethylchroman-4-one [1779];
2,2-dimethyl-7-(2-(neopentylamino)-7H-pyrrolo [2,3-d]pyrimidin-5-yl)chroman-4-one [1780];
(S)-7-(2-(sec-butylamino)-7H-pyrrolo [2,3-d] pyrimidin-5-yl)-2,2-dimethylchroman-4-one [1781];
7-(2-((2-fluoro-2-methylpropyl)amino)-7H-pyrrolo [2,3-d]pyrimidin-5-yl)-2,2-dimethylchroman-4-one [1782];
7-(2-((2,2-difluoropropyl)amino)-7H-pyrrolo [2,3-d] pyrimidin-5-yl)-2,2-dimethylchroman-4-one [1783];
2,2-dimethyl-7-(2-((2,2,2-trifluoroethyl)amino)-7H-pyrrolo [2,3-d] pyrimidin-5-yl)chroman-4-one [1784];
(R)-2,2-dimethyl-7-(2-((1,1,1-trifluoropropan-2-yl)amino)-7H-pyrrolo [2,3-d]pyrimidin-5-yl)chroman-4-one [1785];
(S)-2,2-dimethyl-7-(2-((1,1,1-trifluoropropan-2-yl)amino)-7H-pyrrolo [2,3-d]pyrimidin-5-yl)chroman-4-one [1786];
2,2-dimethyl-7-(2-((3,3,3-trifluoropropyl)amino)-7H-pyrrolo [2,3-d]pyrimidin-5-yl)chroman-4-one [1787];
(S)-7-(2-((l-methoxypropan-2-yl)amino)-7H-pyrrolo [2,3-d]pyrimidin-5-yl)-2,2-dimethylchroman-4-one [1788];
7-(2-ethoxy-7H-pyrrolo [2,3-d] pyrimidin-5-yl)-2,2-dimethylchroman-4-one [1789];
7-(2-(((1-fluorocyclopropyl)methyl)amino)-7H-pyrrolo [2,3-d]pyrimidin-5-yl)-2,2-dimethylchroman-4-one [1790];
(R)-7-(2-((l-cyclopropylethyl)amino)-7H-pyrrolo [2,3-d]pyrimidin-5-yl)-2,2-dimethylchroman-4-one [1791];
2,2-dimethyl-7-(2-(((l-methylcyclopropyl)methyl)amino)-7H-pyrrolo [2,3-d]pyrimidin-5-yl)chroman-4-one [1792];
2,2-dimethyl-7-(2-(((1-(trifluoromethyl)cyclopropyl)methyl)amino)-7H-pyrrolo [2,3-d]pyrimidin-5-yl)chroman-4-one [1793];
7-(2-((2-cyclopropyl-2,2-difluoroethyl)amino)-7H-pyrrolo [2,3-d]pyrimidin-5-yl)-2,2-dimethylchroman-4-one [1794];
7-(2-((3,3-difluorocyclobutyl)amino)-7H-pyrrolo [2,3-d]pyrimidin-5-yl)-2,2-dimethylchroman-4-one [1795];
7-(2-(((1-fluorocyclobutyl)methyl)amino)-7H-pyrrolo [2,3-d]pyrimidin-5-yl)-2,2-dimethylchroman-4-one [1796];
7-(2-((4,4-difluorocyclohexyl)amino)-7H-pyrrolo [2,3-d]pyrimidin-5-yl)-2,2-dimethylchroman-4-one [1797];
7-(2-((cis-4-methoxycyclohexyl)amino)-7H-pyrrolo [2,3-d]pyrimidin-5-yl)-2,2-dimethylchroman-4-one [1798];
7-(2-(((1-fluorocyclohexyl)me thyl)amino)-7H-pyrrolo [2,3-d]pyrimidin-5-yl)-2,2-dimethylchroman-4-one [1799];
7-(2-4(4,4-difluorocyclohexyl)me thyl)amino)-7H-pyrrolo [2,3-d]pyrimidin-5-yl)-2,2-dimethylchroman-4-one [1800];
2,2-dimethyl-7-(2-((1-methylpiperidin-4-yl)amino)-7H-pyrrolo [2,3-d]pyrimidin-5-yl)chroman-4-one [1801];
2,2-dimethyl-7-(2-4(1-methylpipe ridin-4-yl)methyl)amino)-7H-pyrrolo [2,3-d]pyrimidin-5-yl)chroman-4-one [1802];
2,2-dimethyl-7-(2-((tetrahydro -2H-pyran-4-yl)amino)-7H-pyrrolo [2,3-d]pyrimidin-5-yl)chroman-4-one [1803];
2,2-dimethyl-7-(2-43-(4-methylpiperazin-1-yl)phenyl)amino)-7H-pyrrolo [2,3-d]pyrimidin-5-yl)chroman-4-one [1804];
2,2-dimethyl-7-(2-(pyridin-4-ylamino)-7H-pyrrolo [2,3-d]pyrimidin-5-yOchroman-4-one [1805];
2,2-dimethyl-7-(2-42-(4-methylpiperazin-1-yl)pyridin-4-yl)amino)-7H-pyrrolo [2,3-d]pyrimidin-5-yl)chroman-4-one [1806];
2,2-dimethyl-7-(2-46-(4-methylpiperazin-1-yl)pyridin-3-yl)amino)-7H-pyrrolo [2,3-d]pyrimidin-5-yl)chroman-4-one [1807];
2,2-dimethyl-7-(2-(46-(4-methylpiperazin-1-yl)pyridin-3-yl)methyl)amino)-7H-pyrrolo [2,3-d]pyrimidin-5-yl)chroman-4-one [1808];
2,2-dimethyl-7-(2-(quinolin-6-yl)-7H-pyrrolo [2,3-d]pyrimidin-5-yOchroman-4-one [1809];
2,2-dimethyl-7-(44 1-methyl-1H-pyrazol-4-yl)-7H-pyrrolo [2,3-d]pyrimidin-5-yOchroman-4-one [1810];
2,2-dimethyl-7-(4-((trans-4-morpholinocyclohexyl)amino)-7H-pyrrolo [2,3-d]pyrimidin-5-yl)chroman-4-one [1811];
6-(2-isobutyl-7H-pyrrolo [2,3-d]pyrimidin-5-yl)-4,4-dimethyl-3 ,4-dihydroisoquinolin-1 (2H)-one [1813];
4,4-dimethyl-6-(2-(3 ,3 ,3-trifluoropropyl)-7H-pyrrolo [2,3-d]pyrimidin-5-yl)-3 ,4-dihydroisoquinolin-1 (2H)-one [1814];
6-(2-cyclopropyl-7H-pyrrolo [2,3-d]pyrimidin-5-yl)-4,4-dimethyl-3 ,4-dihydroisoquinolin-1 (2H)-one [1815];
6-(2-cyclobutyl-7H-pyrrolo [2,3-d]pyrimidin-5-yl)-4,4-dimethyl-3 ,4-dihydroisoquinolin-1 (2H)-one [1816];
6-(2-(cyclobutylmethyl)-7H-pyrrolo [2,3-d]pyrimidin-5-yl)-4,4-dimethyl-3 ,4-dihydroisoquinolin-1 (2H)-one [1817];
6-(2-amino-7H-pyrrolo [2,3-d] pyrimidin-5-yl)-4,4-dimethyl-3,4-dihydroi soquinolin-1(2H)-one [1818];
4,4-dimethyl-6-(2-(methylamino)-7H-pyrrolo [2,3-d]pyrimidin-5-yl)-3,4-dihydroisoquinolin-1(2H)-one [1819];
6-(2-(ethylamino)-7H-pyrrolo [2,3-d] pyrimidin-5-yl)-4,4-dimethyl-3,4-dihydroi soquinolin-1(2H)-one [1820];
6-(2-(i sop ropylamino)-7H-pyrrolo [2,3-d] pyrimidin-5-yl)-4,4-dimethyl-3,4-dihydroi soquinolin-1(2H)-one [1821];
6-(2-(isobutylamino)-7H-pyrrolo [2,3-d]pyrimidin-5-yl)-4,4-dimethyl-3,4-dihydroi soquinolin-1(2H)-one [1822];
4,4-dimethyl-6-(2-(neopentylamino)-7H-pyrrolo [2,3-d]pyrimidin-5-yl)-3,4-dihydroi soquinolin-1(2H)-one [1823];
(S)-6-(2-(sec-butylamino)-7H-pyrrolo [2,3-d] pyrimidin-5-yl)-4,4-dimethyl-3,4-dihydroisoquinolin-1(2H)-one [1824];
6-(2-((2-fluoro-2-methylpropyl)amino)-7H-pyrrolo [2,3-d]pyrimidin-5-yl)-4,4-dimethyl-3,4-dihydroisoquinolin-1(2H)-one [1825];
6-(2-((2,2-difluoropropyl)amino)-7H-pyrrolo [2,3-d] pyrimidin-5-yl)-4,4-dimethyl-3,4-dihydroisoquinolin-1(2H)-one [1826];
4,4-dimethyl-6-(2-((2,2,2-trifluoroethyDamino)-7H-pyrrolo [2,3-d]pyrimidin-5-yl)-3,4-dihydroisoquinolin-1(2H)-one [1827];
(R)-4,4-dimethyl-6-(2-((1,1,1-trifluoropropan-2-yl)amino)-7H-pyrrolo [2,3-d]pyrimidin-5-yl)-3,4-dihydroisoquinolin-1(2H)-one [1828];
(S)-4,4-dimethyl-6-(2-((1,1,1-trifluoropropan-2-yl)amino)-7H-pyrrolo [2,3-d]pyrimidin-5-yl)-3,4-dihydroisoquinolin-1(2H)-one [1829];
4,4-dimethyl-6-(2-((3,3,3-trifluoropropyl)amino)-7H-pyrrolo [2,3-d]pyrimidin-5-yl)-3,4-dihydroisoquinolin-1(2H)-one [1830];
(S)-6-(2-((l-methoxypropan-2-yl)amino)-7H-pyrrolo [2,3-d]pyrimidin-5-yl)-4,4-dimethyl-3,4-dihydroisoquinolin-1(2H)-one [1831];
6-(2-ethoxy-7H-pyrrolo [2,3-d] pyrimidin-5-yl)-4,4-dimethyl-3,4-dihydroi soquinolin-1(2H)-one [1832];
6-(2-(((1-fluorocyclopropyl)methyl)amino)-7H-pyrrolo [2,3-d] pyrimidin-5-yl)-4,4-dimethyl-3,4-dihydroisoquinolin-1(2H)-one [1833];
(R)-6-(2-((l-cyclopropylethyl)amino)-7H-pyrrolo [2,3-d]pyrimidin-5-yl)-4,4-dimethyl-3,4-dihydroisoquinolin-1(2H)-one [1834];
4,4-dimethyl-6-(2-(((1-methylcyclopropyl)methyl)amino)-7H-pyrrolo [2,3-d]pyrimidin-5-yl)-3,4-dihydroisoquinolin-1(2H)-one [1835];
4,4-dimethyl-6-(2-(41-(trifluoromethyl)cyclopropyl)methyl)amino)-7H-pyrrolo [2,3-d]pyrimidin-5-yl)-3,4-dihydroisoquinolin-1(2H)-one [1836];
6-(2-((2-cyclopropyl-2,2-difluoroethypamino)-7H-pyrrolo [2,3-d]pyrimidin-5-yl)-4,4-dimethyl-3,4-dihydroi soquinolin-1(2H)-one [1837];
6-(2-((3,3-difluorocyclobutyl)amino)-7H-pyrrolo [2,3-d]pyrimidin-5-yl)-4,4-dimethyl-3,4-dihydroisoquinolin-1(2H)-one [1838];
6-(2-(((1-fluorocyclobutyl)methyl)amino)-7H-pyrrolo [2,3-d]pyrimidin-5-yl)-4,4-dimethyl-3,4-dihydroisoquinolin-1(2H)-one [1839];
6-(2-((4,4-difluorocyclohexyl)amino)-7H-pyrrolo [2,3-d]pyrimidin-5-yl)-4,4-dimethyl-3,4-dihydroisoquinolin-1(2H)-one [1840];
6-(2-((cis-4-methoxycyclohexyl)amino)-7H-pyrrolo [2,3-d]pyrimidin-5-yl)-4,4-dimethyl-3,4-dihydroisoquinolin-1(2H)-one [1841];
6-(2-(((1-fluorocyclohexyl)me thyl)amino)-7H-pyrrolo [2,3-d]pyrimidin-5-yl)-4,4-dimethyl-3,4-dihydroisoquinolin-1(2H)-one [1842];
6-(2-(((4,4-difluorocyclohexyl)me thyl)amino)-7H-pyrrolo [2,3-d]pyrimidin-5-yl)-4,4-dimethyl-3,4-dihydroi soquinolin-1(2H)-one [1843];
4,4-dimethyl-6-(2-((1-methylpiperidin-4-yl)amino)-7H-pyrrolo [2,3-d] pyrimidin-5-yl)-3,4-dihydroisoquinolin-1(2H)-one [1844];
4,4-dimethyl-6-(2-(((1-methylpiperidin-4-yl)methyl)amino)-7H-pyrrolo [2,3-d]pyrimidin-5-yl)-3,4-dihydroisoquinolin-1(2H)-one [1845];
4,4-dimethyl-6-(2-((tetrahydro -2H-pyran-4-yl)amino)-7H-pyrrolo [2,3-d]pyrimidin-5-yl)-3,4-dihydroisoquinolin-1(2H)-one [1846];
4,4-dimethyl-6-(2-43-(4-methylpiperazin-1-yl)phenyl)amino)-7H-pyrrolo [2,3-d]pyrimidin-5-yl)-3,4-dihydroi soquinolin-1(2H)-one [1847];
4,4-dimethyl-6-(2-(pyridin-4-ylamino)-7H-pyrrolo [2,3-d]pyrimidin-5-yl)-3,4-dihydroisoquinolin-1(2H)-one [1848];
4,4-dimethyl-6-(2-42-(4-methylpiperazin-1-yl)pyridin-4-yl)amino)-7H-pyrrolo [2,3-d]pyrimidin-5-yl)-3,4-dihydroisoquinolin-1(2H)-one [1849];
4,4-dimethyl-6-(2-46-(4-methylpiperazin-1-yl)pyridin-3-yl)amino)-7H-pyrrolo [2,3-d]pyrimidin-5-yl)-3,4-dihydroisoquinolin-1(2H)-one [1850];
4,4-dimethyl-6-(2-(46-(4-methylpiperazin-1-yl)pyridin-3-yl)methyl)amino)-7H-pyrrolo [2,3-d]pyrimidin-5-yl)-3,4-dihydroi soquinolin-1(2H)-one [1851];
4,4-dimethyl-6-(2-(quinolin-6-yl)-7H-pyrrolo [2,3-d]pyrimidin-5-yl)-3,4-dihydroisoquinolin-1(2H)-one [1852];
4,4-dimethyl-6-(4-(1-methyl-1H-pyrazol-4-yl)-7H-pyrrolo [2,3-d]pyrimidin-5-yl)-3,4-dihydroisoquinolin-1(2H)-one [1853];
4,4-dimethyl-6-(4-((trans-4-morpholinocyclohexyl)amino)-7H-pyrrolo [2,3-d]pyrimidin-5-yl)-3,4-dihydroisoquinolin-1(2H)-one [1854];
6-(2-isobutyl-7H-pyrrolo [2,3-d] pyrimidin-5-yl)quinoline [1856];
6-(2-isobutyl-4-methoxy-7H-pyrrolo [2,3-d] pyrimidin-5-yl)quinoline [1857];
6-(2-(3,3,3-trifluoropropyl)-7H-pyrrolo [2,3-d]pyrimidin-5-yl)quinoline [1858];
6-(2-cyclopropyl-7H-pyrrolo [2,3-d]pyrimidin-5-yl)quinoline [1859];
6-(2-cyclobutyl-7H-pyrrolo [2,3-d]pyrimidin-5-yl)quinoline [1860];
6-(2-(cyclobutylmethyl)-7H-pyrrolo [2,3-d]pyrimidin-5-yl)quinoline [1861];
5-(quinolin-6-yl)-7H-pyrrolo [2,3-d]pyrimidin-2-amine [1862];
N-methyl-5-(quinolin-6-yl)-7H-pyrrolo [2,3-d]pyrimidin-2-amine [1863];
N-ethyl-5-(quinolin-6-yl)-7H-pyrrolo [2,3-d]pyrimidin-2-amine [1864];
N-isopropyl-5-(quinolin-6-yl)-7H-pyrrolo [2,3-d]pyrimidin-2-amine [1865];
N-isobutyl-5-(quinolin-6-yl)-7H-pyrrolo [2,3-d] pyrimidin-2-amine [1866];
N-ne opentyl-5-(quinolin-6-yl)-7H-pyrrolo [2,3-d] pyrimidin-2-amine [1867];
(S)-N-(sec-butyl)-5-(quinolin-6-yl)-7H-pyrrolo [2,3-d]pyrimidin-2-amine [1868];
N-(2-fluoro-2-methylpropyl)-5-(quinolin-6-yl)-7H-pyrrolo [2,3-d]pyrimidin-2-amine [1869];
N-(2,2-difluoropropyl)-5-(quinolin-6-yl)-7H-pyrrolo [2,3-d]pyrimidin-2-amine [1870];
5-(quinolin-6-yl)-N-(2,2,2-trifluoroethyl)-7H-pyrrolo [2,3-d]pyrimidin-2-amine [1871];
(R)-5-(quinolin-6-yl)-N-(1,1,1-trifluoropropan-2-yl)-7H-pyrrolo [2,3-d]pyrimidin-2-amine [1872];
(S)-5-(quinolin-6-yl)-N-(1,1,1-trifluoropropan-2-yl)-7H-pyrrolo [2,3-d] pyrimidin-2-amine [1873];
5-(quinolin-6-yl)-N-(3,3,3-trifluoropropyl)-7H-pyrrolo [2,3-d]pyrimidin-2-amine [1874];
(S)-N-(1-methoxypropan-2-yl)-5-(quinolin-6-yl)-7H-pyrrolo [2,3-d] pyrimidin-2-amine [1875];
6-(2-ethoxy-7H-pyrrolo [2,3-d] pyrimidin-5-yl)quinoline [1876];
N-((1-fluorocyclopropyl)methyl)-5-(quinolin-6-yl)-7H-pyrrolo [2,3-d]pyrimidin-2-amine [1877];
(R)-N-(1-cyclopropylethyl)-5-(quinolin-6-yl)-7H-pyrrolo [2,3-d] pyrimidin-2-amine [1878];
N-((l-methylcyclopropyl)methyl)-5-(quinolin-6-yl)-7H-pyrrolo [2,3-d] pyrimidin-2-amine [1879];
5-(quinolin-6-yl)-N-41-(trifluoromethyl)cyclopropyl)methyl)-7H-pyrrolo [2,3-d]pyrimidin-2-amine [1880];
N-(2-cyclopropyl-2,2-difluoroe thyl)-5-(quinolin-6-yl)-7H-pyrrolo [2,3-d] pyrimidin-2-amine [1881];
N-(3,3-difluorocyclobutyl)-5-(quinolin-6-yl)-7H-pyrrolo [2,3-d]pyrimidin-2-amine [1882];
N-((1-fluorocyclobutypmethyl)-5-(quinolin-6-yl)-7H-pyrrolo [2,3-d]pyrimidin-2-amine [1883];
N-(4,4-difluorocyclohexyl)-5-(quinolin-6-yl)-7H-pyrrolo [2,3-d]pyrimidin-2-amine [1884];
N-(cis-4-methoxycyclohexyl)-5-(quinolin-6-yl)-7H-pyrrolo [2,3-d]pyrimidin-2-amine [1885];
N-((1-fluorocyclohexyl)methyl)-5-(quinolin-6-yl)-7H-pyrrolo [2,3-d]pyrimidin-2-amine [1886];
N-((4,4-difluorocyclohexyl)methyl)-5-(quinolin-6-yl)-7H-pyrrolo [2,3-d]pyrimidin-2-amine [1887];
N-(1-methylpiperidin-4-yl)-5-(quinolin-6-yl)-7H-pyrrolo [2,3-d]pyrimidin-2-amine [1888];
N-((1-methylpiperidin-4-yl)methyl)-5-(quinolin-6-yl)-7H-pyrrolo [2,3-d]pyrimidin-2-amine [1889];
5-(quinolin-6-yl)-N-(tetrahydro-2H-pyran-4-yl)-7H-pyrrolo [2,3-d]pyrimidin-2-amine [1890];
N-(3-(4-methylpiperazin-1-yl)phenyl)-5-(quinolin-6-yl)-7H-pyrrolo [2,3-d]pyrimidin-2-amine [1891];
N-(pyridin-4-yl)-5-(quinolin-6-yl)-7H-pyrrolo [2,3-d]pyrimidin-2-amine [1892];
N-(6-(4-methylpiperazin-1-yl)pyridin-3-yl)-5-(quinolin-6-yl)-7H-pyrrolo [2,3-d]pyrimidin-2-amine [1894];
N-((6-(4-methylpiperazin-1-yl)pyridin-3-yl)methyl)-5-(quinolin-6-yl)-7H-pyrrolo [2,3-d]pyrimidin-2-amine [1895];
6,6′-(7H-pyrrolo [2,3-d] pyrimidine -2,5-diyl)diquinoline [1896];
6-(4-(1-methyl-1H-pyrazol-4-yl)-7H-pyrrolo [2,3-d] pyrimidin-5-yl)quinoline [1897];
N-(trans-4-morpholinocyclohexyl)-5-(quinolin-6-yl)-7H-pyrrolo [2,3-d]pyrimidin-4-amine [1898];
6-(2-methyl-4-((1-methylpiperidin-4-yl)methoxy)-7H-pyrrolo [2,3-d]pyrimidin-5-yl)quinoline [1899];
6-(2-isobutyl-7H-pyrrolo [2,3-d] pyrimidin-5-yl)quinoxaline [1901];
6-(2-(3 ,3,3-trifluoropropyl)-7H-pyrrolo [2,3-d]pyrimidin-5-yl)quinoxaline [1902];
6-(2-cyclopropyl-7H-pyrrolo [2,3-d]pyrimidin-5-yl)quinoxaline [1903];
6-(2-cyclobutyl-7H-pyrrolo [2,3-d]pyrimidin-5-yl)quinoxaline [1904];
6-(2-(cyclobutylmethyl)-7H-pyrrolo [2,3-d]pyrimidin-5-yl)quinoxaline [1905];
5-(quinoxalin-6-yl)-7H-pyrrolo [2,3-d]pyrimidin-2-amine [1906];
N-methyl-5-(quinoxalin-6-yl)-7H-pyrrolo [2,3-d]pyrimidin-2-amine [1907];
N-ethyl-5-(quinoxalin-6-yl)-7H-pyrrolo [2,3-d]pyrimidin-2-amine [1908];
N-isopropyl-5-(quinoxalin-6-yl)-7H-pyrrolo [2,3-d]pyrimidin-2-amine [1909];
N-isobutyl-5-(quinoxalin-6-yl)-7H-pyrrolo [2,3-d]pyrimidin-2-amine [1910];
N-neopentyl-5-(quinoxalin-6-yl)-7H-pyrrolo [2,3-d] pyrimidin-2-amine [1911];
(S)-N-(sec-butyl)-5-(quinoxalin-6-yl)-7H-pyrrolo [2,3-d]pyrimidin-2-amine [1912];
N-(2-fluoro-2-methylpropyl)-5-(quinoxalin-6-yl)-7H-pyrrolo [2,3-d]pyrimidin-2-amine [1913];
N-(2,2-difluoropropyl)-5-(quinoxalin-6-yl)-7H-pyrrolo [2,3-d]pyrimidin-2-amine [1914];
5-(quinoxalin-6-yl)-N-(2,2,2-trifluoroethyl)-7H-pyrrolo [2,3-d]pyrimidin-2-amine [1915];
(R)-5-(quinoxalin-6-yl)-N-(1,1,1-trifluoropropan-2-yl)-7H-pyrrolo [2,3-d]pyrimidin-2-amine [1916];
(S)-5-(quinoxalin-6-yl)-N-(1,1,1-trifluoropropan-2-yl)-7H-pyrrolo [2,3-d]pyrimidin-2-amine [1917];
5-(quinoxalin-6-yl)-N-(3,3,3-trifluoropropyl)-7H-pyrrolo [2,3-d]pyrimidin-2-amine [1918];
(S)-N-(1-methoxypropan-2-yl)-5-(quinoxalin-6-yl)-7H-pyrrolo [2,3-d]pyrimidin-2-amine [1919];
6-(2-ethoxy-7H-pyrrolo [2,3-d] pyrimidin-5-yl)quinoxaline [1920];
N-((1-fluorocyclopropyl)methyl)-5-(quinoxalin-6-yl)-7H-pyrrolo [2,3-d]pyrimidin-2-amine [1921];
(R)-N-(1-cyclopropylethyl)-5-(quinoxalin-6-yl)-7H-pyrrolo [2,3-d]pyrimidin-2-amine [1922];
N-((1-methylcyclopropyl)methyl)-5-(quinoxalin-6-yl)-7H-pyrrolo [2,3-d]pyrimidin-2-amine [1923];
5-(quinoxalin-6-yl)-N-((1-(trifluoromethyl)cyclopropyl)methyl)-7H-pyrrolo [2,3-d]pyrimidin-2-amine [1924];
N-(2-cyclopropyl-2,2-difluoroe thyl)-5-(quinoxalin-6-yl)-7H-pyrrolo [2,3-d]pyrimidin-2-amine [1925];
N-(3,3-difluorocyclobutyl)-5-(quinoxalin-6-yl)-7H-pyrrolo [2,3-d] pyrimidin-2-amine [1926];
N-((1-fluorocyclobutyl)methyl)-5-(quinoxalin-6-yl)-7H-pyrrolo [2,3-d]pyrimidin-2-amine [1927];
N-(4,4-difluorocyclohexyl)-5-(quinoxalin-6-yl)-7H-pyrrolo [2,3-d]pyrimidin-2-amine [1928];
N-(cis-4-methoxycyclohexyl)-5-(quinoxalin-6-yl)-7H-pyrrolo [2,3-d] pyrimidin-2-amine [1929];
N-((1-fluorocyclohexyl)methyl)-5-(quinoxalin-6-yl)-7H-pyrrolo [2,3-d]pyrimidin-2-amine [1930];
N-((4,4-difluorocyclohexyl)methyl)-5-(quinoxalin-6-yl)-7H-pyrrolo [2,3-d]pyrimidin-2-amine [1931];
N-(1-methylpiperidin-4-yl)-5-(quinoxalin-6-yl)-7H-pyrrolo [2,3-d]pyrimidin-2-amine [1932];
N-((1-methylpiperidin-4-yl)methyl)-5-(quinoxalin-6-yl)-7H-pyrrolo [2,3-d] pyrimidin-2-amine [1933];
5-(quinoxalin-6-yl)-N-(tetrahydro-2H-pyran-4-yl)-7H-pyrrolo [2,3-d]pyrimidin-2-amine [1934];
N-(3-(4-methylpiperazin-1-yl)phenyl)-5-(quinoxalin-6-yl)-7H-pyrrolo [2,3-d]pyrimidin-2-amine [1935];
N-(pyridin-4-yl)-5-(quinoxalin-6-yl)-7H-pyrrolo[2,3-d]pyrimidin-2-amine [1936];
N-(2-(4-methylpiperazin-1-yl)pyridin-4-yl)-5-(quinoxalin-6-yl)-7H-pyrrolo [2,3-d]pyrimidin-2-amine [1937];
N-(6-(4-methylpipe razin-1-yl)pyridin-3-yl)-5-(quinoxalin-6-yl)-7H-pyrrolo [2,3-d]pyrimidin-2-amine [1938];
N-((6-(4-methylpiperazin-1-yl)pyridin-3-yl)methyl)-5-(quinoxalin-6-yl)-7H-pyrrolo[2,3-d]pyrimidin-2-amine [1939];
6-(2-(quinolin-6-yl)-7H-pyrrolo[2,3-d]pyrimidin-5-yl)quinoxaline [1940];
6-(4-(1-methyl-1H-pyrazol-4-yl)-7H-pyrrolo[2,3-d]pyrimidin-5-yl)quinoxaline [1941];
N-(trans-4-morpholinocyclohexyl)-5-(quinoxalin-6-yl)-7H-pyrrolo[2,3-d]pyrimidin-4-amine [1942];
6-(2-isobutyl-7H-pyrrolo[2,3-d]pyrimidin-5-yl)quinazoline [1944];
6-(2-(3,3,3-trifluoropropyl)-7H-pyrrolo[2,3-d]pyrimidin-5-yl)quinazoline [1945];
6-(2-cyclopropyl-7H-pyrrolo[2,3-d]pyrimidin-5-yl)quinazoline [1946];
6-(2-cyclobutyl-7H-pyrrolo [2,3-d]pyrimidin-5-yl)quinazoline [1947];
6-(2-(cyclobutylmethyl)-7H-pyrrolo[2,3-d]pyrimidin-5-yl)quinazoline [1948];
5-(quinazolin-6-yl)-7H-pyrrolo[2,3-d]pyrimidin-2-amine [1949];
N-methyl-5-(quinazolin-6-yl)-7H-pyrrolo [2,3-d]pyrimidin-2-amine [1950];
N-ethyl-5-(quinazolin-6-yl)-7H-pyrrolo[2,3-d]pyrimidin-2-amine [1951];
N-isopropyl-5-(quinazolin-6-yl)-7H-pyrrolo [2,3-d] pyrimidin-2-amine [1952];
N-isobutyl-5-(quinazolin-6-yl)-7H-pyrrolo[2,3-d]pyrimidin-2-amine [1953];
N-neopentyl-5-(quinazolin-6-yl)-7H-pyrrolo[2,3-d]pyrimidin-2-amine [1954];
(S)-N-(sec-butyl)-5-(quinazolin-6-yl)-7H-pyrrolo[2,3-d]pyrimidin-2-amine [1955];
N-(2-fluoro-2-methylpropyl)-5-(quinazolin-6-yl)-7H-pyrrolo[2,3-d]pyrimidin-2-amine [1956];
N-(2,2-difluoropropyl)-5-(quinazolin-6-yl)-7H-pyrrolo[2,3-d]pyrimidin-2-amine [1957];
5-(quinazolin-6-yl)-N-(2,2,2-trifluoroethyl)-7H-pyrrolo[2,3-d]pyrimidin-2-amine [1958];
(R)-5-(quinazolin-6-yl)-N-(1,1,1-trifluoropropan-2-yl)-7H-pyrrolo[2,3-d]pyrimidin-2-amine [1959];
(S)-5-(quinazolin-6-yl)-N-(1,1,1-trifluoropropan-2-yl)-7H-pyrrolo[2,3-d]pyrimidin-2-amine [1960];
5-(quinazolin-6-yl)-N-(3,3,3-trifluoropropyl)-7H-pyrrolo[2,3-d]pyrimidin-2-amine [1961];
(S)-N-(1-methoxypropan-2-yl)-5-(quinazolin-6-yl)-7H-pyrrolo[2,3-d]pyrimidin-2-amine [1962];
6-(2-ethoxy-7H-pyrrolo[2,3-d]pyrimidin-5-yl)quinazoline [1963];
N-((1-fluorocyclopropyl)methyl)-5-(quinazolin-6-yl)-7H-pyrrolo[2,3-d]pyrimidin-2-amine [1964];
(R)-N-(1-cyclopropylethyl)-5-(quinazolin-6-yl)-7H-pyrrolo[2,3-d]pyrimidin-2-amine [1965];
N-((1-methylcyclopropyl)methyl)-5-(quinazolin-6-yl)-7H-pyrrolo[2,3-d]pyrimidin-2-amine [1968];
5-(quinazolin-6-yl)-N-((1-(trifluoromethyl)cyclopropyl)methyl)-7H-pyrrolo[2,3-d]pyrimidin-2-amine [1967];
N-(2-cyclopropyl-2,2-difluoroe thyl)-5-(quinazolin-6-yl)-7H-pyrrolo [2,3-d] pyrimidin-2-amine [1974];
N-(3,3-difluorocyclobutyl)-5-(quinazolin-6-yl)-7H-pyrrolo[2,3-d]pyrimidin-2-amine [1969];
N-((1-fluorocyclobutypmethyl)-5-(quinazolin-6-yl)-7H-pyrrolo[2,3-d]pyrimidin-2-amine [1970];
N-(4,4-difluorocyclohexyl)-5-(quinazolin-6-yl)-7H-pyrrolo[2,3-d]pyrimidin-2-amine [1971];
N-(cis-4-methoxycyclohexyl)-5-(quinazolin-6-yl)-7H-pyrrolo[2,3-d]pyrimidin-2-amine [1972];
N-((1-fluorocyclohexyl)methyl)-5-(quinazolin-6-yl)-7H-pyrrolo[2,3-d]pyrimidin-2-amine [1973];
N-((4,4-difluorocyclohexyl)methyl)-5-(quinazolin-6-yl)-7H-pyrrolo[2,3-d]pyrimidin-2-amine [1974];
N-(1-methylpiperidin-4-yl)-5-(quinazolin-6-yl)-7H-pyrrolo[2,3-d]pyrimidin-2-amine [1975];
N-((1-methylpiperidin-4-yl)methyl)-5-(quinazolin-6-yl)-7H-pyrrolo [2,3-d]pyrimidin-2-amine [1976];
5-(quinazolin-6-yl)-N-(tetrahydro-2H-pyran-4-yl)-7H-pyrrolo[2,3-d]pyrimidin-2-amine [1977];
N-(3-(4-methylpiperazin-1-yl)phenyl)-5-(quinazolin-6-yl)-7H-pyrrolo[2,3-d]pyrimidin-2-amine [1978];
N-(pyridin-4-yl)-5-(quinazolin-6-yl)-7H-pyrrolo[2,3-d]pyrimidin-2-amine [1979];
N-(2-(4-methylpiperazin-1-yl)pyridin-4-yl)-5-(quinazolin-6-yl)-7H-pyrrolo[2,3-d]pyrimidin-2-amine [1980];
N-(6-(4-methylpiperazin-1-yl)pyridin-3-yl)-5-(quinazolin-6-yl)-7H-pyrrolo[2,3-d]pyrimidin-2-amine [1981];
N-((6-(4-methylpiperazin-1-yl)pyridin-3-yl)methyl)-5-(quinazolin-6-yl)-7H-pyrrolo[2,3-d]pyrimidin-2-amine [1982];
6-(2-(quinolin-6-yl)-7H-pyrrolo[2,3-d]pyrimidin-5-yl)quinazoline [1983];
6-(4-(1-methyl-1H-pyrazol-4-yl)-7H-pyrrolo[2,3-d]pyrimidin-5-yl)quinazoline [1984];
N-(trans-4-morpholinocyclohexyl)-5-(quinazolin-6-yl)-7H-pyrrolo[2,3-d]pyrimidin-4-amine [1985];
5-(4-(4-methylpiperazin-1-yl)quinazolin-6-yl)-7H-pyrrolo[2,3-d]pyrimidin-2-amine [1986];
6-(2-cyclopropyl-7H-pyrrolo[2,3-d]pyrimidin-5-yl)-4-(4-methylpiperazin-1-yl)quinazoline [1987];
7-(2-isobutyl-7H-pyrrolo[2,3-d]pyrimidin-5-yl)-2-((1-methylpiperidin-4-yl)oxy)quinoxaline
2-((l-methylpiperidin-4-yl)oxy)-7-(2-(3,3,3-trifluoropropyl)-7H-pyrrolo[2,3-d]pyrimidin-5-yl)quinoxaline [1989];
7-(2-cyclopropyl-7H-pyrrolo [2,3-d] pyrimidin-5-yl)-2-((1-methylpipe ridin -4-yl)oxy)quinoxaline [1991];
7-(2-cyclobutyl-7H-pyrrolo [2,3-d] pyrimidin -5-yl)-2-((1-methylpiperidin-4-yl)oxy)quinoxaline [1992];
7-(2-(cyclobutylmethyl)-7H-pyrrolo [2,3-d] pyrimidin-5-yl)-2-((1-methylpiperidin-4-yl)oxy)quinoxaline [1993];
5-(3-((l-methylpiperidin-4-yl)oxy)quinoxalin-6-yl)-7H-pyrrolo[2,3-d]pyrimidin-2-amine [1994];
N-methyl-5-(3-((1-methylpiperidin-4-yl)oxy)quinoxalin-6-yl)-7H-pyrrolo [2,3-d]pyrimidin-2-amine [1995];
N-ethyl-5-(3-((l-methylpiperidin -4-yl)oxy)quinoxalin-6-yl) -7H-pyrrolo [2,3-d] pyrimidin-2-amine [1996];
N-isopropyl-5-(3-((1-methylpiperidin-4-yl)oxy)quinoxalin-6-yl)-7H-pyrrolo [2,3-d]pyrimidin-2-amine [1997];
N-i sobutyl-5-(3-((1-methylpiperidin-4-yl)oxy)quinoxalin-6-yl) -7H-pyrrolo [2,3-d] pyrimidin-2-amine [1998];
5-(3-((l-methylpiperidin-4-yl)oxy)quinoxalin-6-yl)-N-neopentyl-7H-pyrrolo [2,3-d]pyrimidin-2-amine [1999]; and
(S)-N-(sec-butyl)-5-(3-((1-methylpiperidin-4-yl)oxy)quinoxalin-6-yl)-7H-pyrrolo [2,3-d]pyrimidin-2-amine [2000]; or a pharmaceutically acceptable salt thereof.

27. The compound of claim 1, wherein the compound of Formula I is selected from the group consisting of:
N-(2-fluoro-2-methylpropyl)-5-(3-((1-methylpiperidin-4-yl)oxy)quinoxalin-6-yl)-7H-pyrrolo [2,3-d]pyrimidin-2-amine [2001];
N-(2,2-difluoropropyl)-5-(3-((1-methylpiperidin-4-yl)oxy)quinoxalin-6-yl)-7H-pyrrolo [2,3-d]pyrimidin-2-amine [2002];
5-(3-((l-methylpipe ridin-4-yl)oxy)quinoxalin -6-yl)-N-(2,2,2-trifluoroe thyl)-7H-pyrrolo [2,3-d]pyrimidin-2-amine [2003];
(R)-5-(3-((l-me thylpiperidin-4-yl)oxy)quinoxalin-6-yl)-N-(1,1,1-trifluoropropan-2-yl)-7H-pyrrolo [2,3-d]pyrimidin-2-amine [2004];
(S)-5-(3-((l-methylpipe ridin-4-yl)oxy)quinoxalin-6-yl)-N-(1,1,1-trifluoropropan-2-yl)-7H-pyrrolo [2,3-d]pyrimidin-2-amine [2005];
5-(3-((l-methylpipe ridin-4-yl)oxy)quinoxalin-6-yl)-N-(3,3,3-trifluoropropyl)-7H-pyrrolo [2,3-d]pyrimidin-2-amine [2006];
(S)-N-(1-methoxypropan-2-yl)-5-(3-((l-me thylpiperidin-4-yl)oxy)quinoxalin-6-yl)-7H-pyrrolo [2,3-d]pyrimidin-2-amine [2007];
7-(2-ethoxy-7H-pyrrolo [2,3-d] pyrimidin-5-yl)-2-((1-methylpiperidin-4-yl)oxy)quinoxaline [2008];
N-((1-fluorocyclopropyl)methyl)-5-(3-((1-methylpipe ridin-4-yl)oxy)quinoxalin-6-yl)-7H-pyrrolo [2,3-d]pyrimidin-2-amine [2009];
(R)-N-(1-cyclopropylethyl)-5-(3-((1-me thylpiperidin-4-yl)oxy)quinoxalin-6-yl)-7H-pyrrolo [2,3-d]pyrimidin-2-amine [2010];
N-((1-methylcyclopropyl)methyl)-5-(3-((1-me thylpiperidin-4-yl)oxy)quinoxalin-6-yl)-7H-pyrrolo [2,3-d]pyrimidin-2-amine [2011];
5-(3-((l-methylpipe ridin-4-yl)oxy)quinoxalin-6-yl)-N-((1-(trifluoromethyl)cyclopropyl)methyl)-7H-pyrrolo [2,3-d]pyrimidin-2-amine [2012];
N-(2-cyclopropyl-2,2-difluoroe thyl)-5-(3-((1-me thylpiperidin-4-yl)oxy)quinoxalin-6-yl)-7H-pyrrolo [2,3-d]pyrimidin-2-amine [2013];
N-(3,3-difluorocyclobutyl)-5-(3-((l-methylpiperidin-4-yl)oxy)quinoxalin-6-yl)-7H-pyrrolo [2,3-d]pyrimidin-2-amine [2014];
N-((1-fluorocyclobutyl)me thyl)-5-(3-((l-methylpiperidin-4-yl)oxy)quinoxalin-6-yl)-7H-pyrrolo [2,3-d]pyrimidin-2-amine [2015];
N-(4,4-difluorocyclohexyl)-5-(3-((l-methylpiperidin-4-yl)oxy)quinoxalin-6-yl)-7H-pyrrolo [2,3-d]pyrimidin-2-amine [2016];
N-(cis-4-methoxycyclohexyl)-5-(3-((l-methylpiperidin-4-yl)oxy)quinoxalin-6-yl)-7H-pyrrolo [2,3-d]pyrimidin-2-amine [2017];
N-((1-fluorocyclohexyl)methyl)-5-(3-((1-me thylpiperidin-4-yl)oxy)quinoxalin-6-yl)-7H-pyrrolo [2,3-d]pyrimidin-2-amine [2018];
N-((4,4-difluorocyclohexyl)methyl)-5-(3-((l-me thylpiperidin-4-yl)oxy)quinoxalin-6-yl)-7H-pyrrolo [2,3-d]pyrimidin-2-amine [2019];
N-(1-methylpiperidin-4-yl)-5-(3-((l-me thylpiperidin-4-yl)oxy)quinoxalin-6-yl)-7H-pyrrolo [2,3-d]pyrimidin-2-amine [2020];
N-((1-methylpiperidin-4-yl)methyl)-5-(3-((1-methylpiperidin-4-yl)oxy)quinoxalin-6-yl)-7H-pyrrolo [2,3-d]pyrimidin-2-amine [2021];
5-(3-((l-methylpipe ridin-4-yl)oxy)quinoxalin-6-yl)-N-(tetrahydro-2H-pyran-4-yl)-7H-pyrrolo [2,3-d]pyrimidin-2-amine [2022];
N-(3-(4-methylpiperazin-1-yl)phenyl)-5-(3-((1-methylpiperidin-4-ypoxy)quinoxalin-6-yl)-7H-pyrrolo [2,3-d]pyrimidin-2-amine [2023];
5-(3-((l-methylpipe ridin-4-yl)oxy)quinoxalin-6-yl)-N-(pyridin-4-yl)-7H-pyrrolo [2,3-d]pyrimidin-2-amine [2024];
N-(2-(4-methylpiperazin-1-yl)pyridin-4-yl)-5-(3-((1-me thylpiperidin-4-yl)oxy)quinoxalin-6-yl)-7H-pyrrolo [2,3-d]pyrimidin-2-amine [2025];
N-(6-(4-methylpiperazin-1-yl)pyridin-3-yl)-5-(3-((1-me thylpiperidin-4-yl)oxy)quinoxalin-6-yl)-7H-pyrrolo [2,3-d]pyrimidin-2-amine [2026];
N-((6-(4-methylpiperazin-1-yl)pyridin-3-yl)methyl)-5-(3-((1-me thylpiperidin-4-yl)oxy)quinoxalin-6-yl)-7H-pyrrolo [2,3-d] pyrimidin-2-amine [2027];
2-((l-methylpiperidin-4-yl)oxy)-7-(2-(quinolin-6-yl)-7H-pyrrolo [2,3-d]pyrimidin-5-yl)quinoxaline [2028];
7-(4-(1-methyl-1H-pyrazol-4-yl)-7H-pyrrolo [2,3-d] pyrimidin-5-yl)-2-((1-methylpiperidin-4-yl)oxy)quinoxaline [2029];
5-(3-((l-methylpipe ridin-4-yl)oxy)quinoxalin-6-yl)-N-(trans-4-morpholinocyclohexyl)-7H-pyrrolo [2,3-d]pyrimidin-4-amine [2030];
N-(2-(4-methylpiperazin-1-yl)pyridin-4-yl)-5-(3-(pyridin-3-yl)pyrazolo [1,5-a]pyridin-5-yl)-7H-pyrrolo [2,3-d]pyrimidin-2-amine [2031];
(5-(2-(methylamino)-7H-pyrrolo [2,3-d]pyrimidin-5-yl)pyrazolo [1,5-a]pyridin-3-yl)(4-methylpiperazin-1-yl)methanone [2032];
N-(cis-3-methoxycyclobutyl)-5-(2-methylbenzo [d]thiazol-6-yl)-7H-pyrrolo [2,3-d]pyrimidin-2-amine [2033];
N-(cis-3-methoxycyclobutyl)-5-(thieno [3,2-c]pyridin-2-yl)-7H-pyrrolo [2,3-d]pyrimidin-2-amine ;
5-(imidazo [1,2-a] pyridin-6-yl)-N-(cis-3-methoxycyclobutyl)-7H-pyrrolo [2,3-d]pyrimidin-2-amine [2035];
5-(3-fluoroimidazo [1,2-a]pyridin-6-yl)-N-(cis-3-methoxycyclobutyl)-7H-pyrrolo [2,3-d]pyrimidin-2-amine [2036];
5-(8-fluoroimidazo [1,2-a]pyridin-6-yl)-N-(cis-3-methoxycyclobutyl)-7H-pyrrolo [2,3-d]pyrimidin-2-amine [2037];
5-(8-fluoro-2-methylimidazo [1,2-a] pyridin-6-yl)-N-(cis-3-methoxycyclobutyl)-7H-pyrrolo [2,3-d]pyrimidin-2-amine [2038];
5-(8-fluoro-3-methylimidazo [1,2-a] pyridin-6-yl)-N-(cis-3-methoxycyclobutyl)-7H-pyrrolo [2,3-d]pyrimidin-2-amine [2039];
5-(3-(difluoromethyl)imidazo[1,2-a]pyridin-6-yl)-N-(cis-3-methoxycyclobutyl)-7H-pyrrolo [2,3-d]pyrimidin-2-amine [2040];
(8-fluoro-6-(2-((cis-3-methoxycyclobutyl)amino)-7H-pyrrolo [2,3-d]pyrimidin-5-yl)imidazo [1,2-a] pyridin-3-yl)methanol [2041];
N-(2,2-difluoroethyl)-6-(2-((cis-3-methoxycyclobutyl)amino)-7H-pyrrolo [2,3-d]pyrimidin-5-yl)imidazo[1,2-a]pyridine-3-carboxamide [2042];
5-(imidazo [1,2-a] pyrimidin-6-yl)-N-(cis-3-methoxycyclobutyl)-7H-pyrrolo [2,3-d]pyrimidin-2-amine [2043];
5-(4-fluoro-14 sop ropyl-2-methyl-1H-benzo [d]imidazol-6-yl)-N-(cis-3-methoxycyclobutyl)-7H-pyrrolo [2,3-d]pyrimidin-2-amine [2044];
5-(4-fluoro-2-methyl-1-(1-methylpiperidin-4-yl)-1H-benzo [d] imidazol-6-yl)-N-(cis-3-methoxycyclobutyl)-7H-pyrrolo [2,3-d] pyrimidin-2-amine [2045];
5-(14 sopropyl-2-methyl-1H-imidazo [4,5-b]pyridin-6-yl)-N-(cis-3-methoxycyclobutyl)-7H-pyrrolo [2,3-d]pyrimidin-2-amine [2046];
5-(3-(2,2-difluoroethyl)-2-methyl-3H-imidazo[4,5-b]pyridin-5-yl)-N-(cis-3-methoxycyclobutyl)-7H-pyrrolo [2,3-d]pyrimidin-2-amine [2047];
5-(3 sopropyl-2-methyl-3H-imidazo [4,5-b]pyridin-5-yl)-N-(cis-3-methoxycyclobutyl)-7H-pyrrolo [2,3-d]pyrimidin-2-amine [2048];
5-([1,2,4]triazolo [1,5-a]pyridin-6-yl)-N-(cis-3-methoxycyclobutyl)-7H-pyrrolo [2,3-d]pyrimidin-2-amine [2049];
5-(8-fluoro-[1,2,4]triazolo [1,5-a]pyridin-6-yl)-N-(cis-3-methoxycyclobutyl)-7H-pyrrolo [2,3-d]pyrimidin-2-amine [2050];
N-(cis-3-methoxycyclobutyl)-5-(1-methyl-1H-benzo[d] [1,2,3]triazol-6-yl)-7H-pyrrolo [2,3-d]pyrimidin-2-amine [2051];
5-(1-ethyl-1H-benzo [d] [1,2,3]triazol-6-yl)-N-(cis-3-methoxycyclobutyl)-7H-pyrrolo [2,3-d]pyrimidin-2-amine [2052];
5-(imidazo [1,2-b]pyridazin-6-yl)-N-(cis-3-methoxycyclobutyl)-7H-pyrrolo [2,3-d]pyrimidin-2-amine [2053];
5-(3-chloroimidazo [1,2-b]pyridazin-6-yl)-N-(cis-3-methoxycyclobutyl)-7H-pyrrolo [2,3-d]pyrimidin-2-amine [2054];
N-(cis-3-methoxycyclobutyl)-5-(2-methylimidazo [1,2-b]pyridazin-6-yl)-7H-pyrrolo [2,3-d]pyrimidin-2-amine [2055];
N-(cis-3-methoxycyclobutyl)-5-(3-methylimidazo [1,2-b]pyridazin-6-yl)-7H-pyrrolo [2,3-d]pyrimidin-2-amine [2056];
5-(3-(difluoromethyl)imidazo[1,2-b]pyridazin-6-yl)-N-(cis-3-methoxycyclobutyl)-7H-pyrrolo [2,3-d]pyrimidin-2-amine [2057];
N-(cis-3-methoxycyclobutyl)-5-(pyrazolo [1,5-a]pyridin-5-yl)-7H-pyrrolo [2,3-d]pyrimidin-2-amine [2058];
N-(cis-3-methoxycyclobutyl)-5-(3-(1-methyl-1H-pyrazol-4-yl)pyrazolo [1,5-a]pyridin-5-yl)-7H-pyrrolo [2,3-d]pyrimidin-2-amine [2059];
(5-(2-((cis-3-methoxycyclobutyl)amino)-7H-pyrrolo [2,3-d] pyrimidin-5-yl)pyrazolo [1,5-a] pyridin-3-yl)(piperidin-1-yl)methanone [2060];
N-(2,2-difluoroethyl)-5-(2-((cis-3-methoxycyclobutyl)amino)-7H-pyrrolo [2,3-d]pyrimidin-5-yl)pyrazolo [1,5-a]pyridine-3-carboxamide [2061];
N-(2-fluoro-2-methylpropyl)-5-(2-((cis-3-methoxycyclobutyl)amino)-7H-pyrrolo [2,3-d]pyrimidin-5-yl)pyrazolo [1,5-a]pyridine-3-carboxamide [2062];
5-(2-((cis-3-methoxycyclobutyl)amino)-7H-pyrrolo [2,3-d]pyrimidin-5-yl)-N-((R)-1, 1, 1-trifluoropropan-2-yl)pyrazolo [1,5-a]pyridine-3-carboxamide [2063];
N-(3,3-difluorocyclobutyl)-5-(2-((cis-3-methoxycyclobutyl)amino)-7H-pyrrolo [2,3-d]pyrimidin-5-yl)pyrazolo[1,5-a]pyridine-3-carboxamide [2064];
N-((3-difluorocyclobutypmethyl)-5-(2-((cis-3-methoxycyclobutypamino)-7H-pyrrolo [2,3-d]pyrimidin-5-yl)pyrazolo [1,5-a]pyridine-3-carboxamide [2065];
5-(2-((cis-3-methoxycyclobutyl)amino)-7H-pyrrolo [2,3-d]pyrimidin-5-yl)-N-(cis-4-methoxycyclohexyl)pyrazolo [1,5-a] pyridine-3-carboxamide [2066];
5-(2-((cis-3-methoxycyclobutyl)amino)-7H-pyrrolo [2,3-d]pyrimidin-5-yl)-N-(1-methylpiperidin-4-yl)pyrazolo [1,5-a]pyridine-3-carboxamide [2067];
5-(2-((cis-3-methoxycyclobutyl)amino)-7H-pyrrolo [2,3-d]pyrimidin-5-yl)-N-(tetrahydro-2H-pyran-4-yl)pyrazolo [1,5-a] pyridine-3-carboxamide [2068];
5-(2-((cis-3-methoxycyclobutyl)amino)-7H-pyrrolo [2,3-d]pyrimidin-5-yl)-N-(pyridin-3-yl)pyrazolo [1,5-a]pyridine-3-carboxamide [2069];
N-(cis-3-methoxycyclobutyl)-5-(pyrazolo [1,5-a]pyrimidin-5-yl)-7H-pyrrolo [2,3-d]pyrimidin-2-amine [2070];
5-(3-cyclopropylpyrazolo [1,5-a] pyrimidin-5-yl)-N-(cis-3-methoxycyclobutyl)-7H-pyrrolo [2,3-d]pyrimidin-2-amine [2071];
8-(2-((cis-3-methoxycyclobutyl)amino)-7H-pyrrolo [2,3-d]pyrimidin-5-yl)-3,4-dihydrobenzo [f] [1,4] oxazepin-5(2H)-one [2072];
7-(2-((cis-3-methoxycyclobutyl)amino)-7H-pyrrolo [2,3-d]pyrimidin-5-yl)-2,2-dimethylchroman-4-one [2073];
6-(2-((cis-3-methoxycyclobutyl)amino)-7H-pyrrolo [2,3-d]pyrimidin-5-yl)-4,4-dimethyl-3,4-dihydroisoquinolin-1(2H)-one [2074];
N-(cis-3-methoxycyclobutyl)-5-(quinolin-6-yl)-7H-pyrrolo [2,3-d]pyrimidin-2-amine [2075];
N-(cis-3-methoxycyclobutyl)-5-(quinoxalin-6-yl)-7H-pyrrolo [2,3-d]pyrimidin-2-amine [2076];
N-(cis-3-methoxycyclobutyl)-5-(quinazolin-6-yl)-7H-pyrrolo [2,3-d]pyrimidin-2-amine [2077];
N-(cis-3-methoxycyclobutyl)-5-(3-((1-methylpiperidin-4-yl)oxy)quinoxalin-6-yl)-7H-pyrrolo [2,3-d]pyrimidin-2-amine [2078];
5-(2-methylbenzo [d]thiazol-6-yl)-N-(ci s-4-(trifluoromethoxy)cyclohexyl)-7H-pyrrolo [2,3-d]pyrimidin-2-amine [2079];
5-(thieno [3,2-c]pyridin-2-yl)-N-(cis-4-(trifluoromethoxy)cyclohexyl)-7H-pyrrolo [2,3-d]pyrimidin-2-amine [2080];
5-(imidazo [1,2-a]pyridin-6-yl)-N-(cis-4-(trifluoromethoxy)cyclohexyl)-7H-pyrrolo [2,3-d]pyrimidin-2-amine [2081];
5-(3-fluoroimidazo [1,2-a]pyridin-6-yl)-N-(cis-4-(trifluoromethoxy)cyclohexyl)-7H-pyrrolo [2,3-d]pyrimidin-2-amine [2082];
5-(8-fluoroimidazo [1,2-a]pyridin-6-yl)-N-(cis-4-(trifluoromethoxy)cyclohexyl)-7H-pyrrolo [2,3-d]pyrimidin-2-amine [2083];
5-(8-fluoro-2-methylimidazo [1,2-a]pyridin-6-yl)-N-(cis-4-(trifluoromethoxy)cyclohexyl)-7H-pyrrolo [2,3-d]pyrimidin-2-amine [2084];
5-(8-fluoro-3-methylimidazo [1,2-a]pyridin-6-yl)-N-(cis-4-(trifluoromethoxy)cyclohexyl)-7H-pyrrolo [2,3-d]pyrimidin-2-amine [2085];
5-(3-(difluoromethyl)imidazo [1,2-a]pyridin-6-yl)-N-(cis-4-(trifluoromethoxy)cyclohexyl)-7H-pyrrolo [2,3-d]pyrimidin-2-amine [2086];
(8-fluoro-6-(2-((cis-4-(trifluoromethoxy)cyclohexyl)amino)-7H-pyrrolo [2,3-d]pyrimidin-5-yl)imidazo [1,2-a]pyridin-3-yl)methanol [2087];
N-(2,2-difluoroethyl)-6-(2-((cis-4-(trifluoromethoxy)cyclohexyl)amino)-7H-pyrrolo [2,3-d]pyrimidin-5-yl)imidazo [1,2-a]pyridine-3-carboxamide [2088];
5-(imidazo [1,2-a]pyrimidin-6-yl)-N-(cis-4-(trifluoromethoxy)cyclohexyl)-7H-pyrrolo [2,3-d]pyrimidin-2-amine [2089];
5-(4-fluoro-14 sop ropyl-2-methyl-1H-benzo [d] imidazol-6-yl)-N-(cis-4-(trifluoromethoxy) cyclohexyl)-7H-pyrrolo [2,3-d] pyrimidin-2-amine [2090];
5-(4-fluoro-2-methyl-1-(1-methylpiperidin-4-yl)-1H-benzo [d] imidazol-6-yl)-N-(cis-4-(trifluoromethoxy)cyclohexyl)-7H-pyrrolo [2,3-d] pyrimidin-2-amine [2091];
5-(1-i sopropyl-2-methyl-1H-imidazo [4,5-b]pyridin-6-yl)-N-(cis-4-(trifluoromethoxy) cyclohexyl)-7H-pyrrolo [2,3-d] pyrimidin-2-amine [2092];
5-(3-(2,2-difluoroethyl)-2-methyl-3H-imidazo [4,5-b] pyridin-5-yl)-N-(cis-4-(trifluoromethoxy) cyclohexyl)-7H-pyrrolo [2,3-d] pyrimidin-2-amine [2093];
5-(3-i sopropyl-2-methyl-3H-imidazo [4,5-b]pyridin-5-yl)-N-(cis-4-(trifluoromethoxy) cyclohexyl)-7H-pyrrolo [2,3-d] pyrimidin-2-amine [2094];
5-([1,2,4]triazolo [1,5-a]pyridin-6-yl)-N-(cis-4-(trifluoromethoxy)cyclohexyl)-7H-pyrrolo [2,3-d]pyrimidin-2-amine [2095];
5-(8-fluoro-[1,2,4]triazolo [1,5-a]pyridin-6-yl)-N-(cis-4-(trifluoromethoxy)cyclohexyl)-7H-pyrrolo [2,3-d]pyrimidin-2-amine [2096];
5-(1-methyl-1H-benzo [d] [1,2,3]triazol-6-yl)-N-(cis-4-(trifluoromethoxy)cyclohexyl)-7H-pyrrolo [2,3-d]pyrimidin-2-amine [2097];
5-(1-ethyl-1H-benzo [d] [1,2,3]triazol-6-yl)-N-(cis-4-(trifluoromethoxy)cyclohexyl)-7H-pyrrolo [2,3-d]pyrimidin-2-amine [2098];
5-(imidazo [1,2-b]pyridazin-6-yl)-N-(cis-4-(trifluoromethoxy)cyclohexyl)-7H-pyrrolo [2,3-d]pyrimidin-2-amine [2099];
5-(3-chloroimidazo [1,2-b] pyridazin-6-yl)-N-(cis-4-(trifluoromethoxy)cyclohexyl)-7H-pyrrolo [2,3-d]pyrimidin-2-amine [2100];
5-(2-methylimidazo [1,2-b] pyridazin-6-yl)-N-(cis-4-(trifluoromethoxy)cyclohexyl)-7H-pyrrolo [2,3-d]pyrimidin-2-amine [2101];
5-(3-methylimidazo [1,2-b] pyridazin-6-yl)-N-(cis-4-(trifluoromethoxy)cyclohexyl)-7H-pyrrolo [2,3-d]pyrimidin-2-amine [2102];
5-(3-(difluoromethyl)imidazo [1,2-b]pyridazin-6-yl)-N-(cis-4-(trifluoromethoxy)cyclohexyl)-7H-pyrrolo [2,3-d]pyrimidin-2-amine [2103];
5-(pyrazolo [1,5-a]pyridin-5-yl)-N-(cis-4-(trifluoromethoxy)cyclohexyl)-7H-pyrrolo [2,3-d]pyrimidin-2-amine [2104];
5-(3-(1-methyl-1H-pyrazol-4-yl)pyrazolo [1,5-a]pyridin-5-yl)-N-(cis-4-(trifluo romethoxy) cyclohexyl)-7H-pyrrolo [2,3-d] pyrimidin-2-amine [2105];
piperidin-1-yl(5-(2-((cis-4-(trifluoromethoxy)cyclohexyl)amino)-7H-pyrrolo [2,3-d] pyrimidin-5-yl)pyrazolo [1,5-a]pyridin-3-yl)methanone [2106];
N-(2,2-difluoroethyl)-5-(2-((cis-4-(trifluoromethoxy)cyclohexyl)amino)-7H-pyrrolo [2,3]-5-yl)pyrazolo [1,5-a]pyridine-3-carboxamide [2107];
N-(2-fluoro-2-methylpropyl)-5-(2-((cis-4-(trifluoromethoxy)cyclohexyl)amino)-7H-pyrrolo [2,3-d]pyrimidin-5-yl)pyrazolo [1,5-a]pyridine-3-carboxamide [2108];
5-(2-((cis-4-(trifluoromethoxy)cyclohexyl)amino)-7H-pyrrolo [2,3-d]pyrimidin-5-yl)-N-((R)-1,1,1-trifluoropropan-2-yl)pyrazolo [1,5-a]pyridine-3-carboxamide [2109];
N-(3,3-difluorocyclobutyl)-5-(2-((cis-4-(trifluoromethoxy)cyclohexyl)amino)-7H-pyrrolo [2,3-d]pyrimidin-5-yl)pyrazolo [1,5-a]pyridine-3-carboxamide [2110];
N-((3,3-difluorocyclobutyl)methyl)-5-(2-((cis-4-(trifluoromethoxy)cyclohexyl)amino)-7H-pyrrolo [2,3-d]pyrimidin-5-yl)pyrazolo [1,5-a]pyridine-3-carboxamide [2111];
N-((1r,4r)-4-methoxycyclohexyl)-5-(2-((cis-4-(trifluoromethoxy)cyclohexyl)amino)-7H-pyrrolo [2,3-d]pyrimidin-5-yl)pyrazolo [1,5-a]pyridine-3-carboxamide [2112];
N-(1-methylpiperidin-4-yl)-5-(2-((cis-4-(trifluoromethoxy)cyclohexyl)amino)-7H-pyrrolo [2,3-d]pyrimidin-5-yl)pyrazolo [1,5-a]pyridine-3-carboxamide [2113];
N-(tetrahydro-2H-pyran-4-yl)-5-(2-((cis-4-(trifluoromethoxy)cyclohexyl)amino)-7H-pyrrolo [2,3-d]pyrimidin-5-yl)pyrazolo [1,5-a]pyridine-3-carboxamide [2114];
N-(pyridin-3-yl)-5-(2-((cis-4-(trifluoromethoxy)cyclohexyl)amino)-7H-pyrrolo [2,3-d]pyrimidin-5-yl)pyrazolo[1,5-a]pyridine-3-carboxamide [2115];
5-(pyrazolo [1,5-a]pyrimidin-5-yl)-N-(cis-4-(trifluoromethoxy)cyclohexyl)-7H-pyrrolo [2,3-d]pyrimidin-2-amine [2116];
5-(3-cyclopropylpyrazolo [1,5-a] pyrimidin-5-yl)-N-(cis-4-(trifluoromethoxy)cyclohexyl)-7H-pyrrolo [2,3-d]pyrimidin-2-amine [2117];
8-(2-((cis-4-(trifluoromethoxy)cyclohexyl)amino)-7H-pyrrolo [2,3-d]pyrimidin-5-yl)-3,4-dihydrobenzo [f] [1,4] oxazepin-5(2H)-one [2118];
2,2-dimethyl-7-(2-((cis-4-(trifluoromethoxy)cyclohexyl)amino)-7H-pyrrolo [2,3-d]pyrimidin-5-yl)chroman-4-one [2119];
4,4-dimethyl-6-(2-((cis-4-(trifluoromethoxy)cyclohexyl)amino)-7H-pyrrolo [2,3-d]pyrimidin-5-yl)-3,4-dihydroi soquinolin-1(2H)-one [2120];
5-(quinolin-6-yl)-N-(cis-4-(trifluoromethoxy)cyclohexyl)-7H-pyrrolo [2,3-d]pyrimidin-2-amine [2121];
5-(quinoxalin-6-yl)-N-(cis-4-(trifluoromethoxy)cyclohexyl)-7H-pyrrolo [2,3-d]pyrimidin-2-amine [2122];
5-(quinazolin-6-yl)-N-(cis-4-(trifluoromethoxy)cyclohexyl)-7H-pyrrolo [2,3-d]pyrimidin-2-amine [2123];
5-(3-((l-methylpipe ridin-4-yl)oxy)quinoxalin-6-yl)-N-(cis-4-(trifluoromethoxy)cyclohexyl)-7H-pyrrolo [2,3-d]pyrimidin-2-amine [2124];
N-(cis-4-ethoxycyclohexyl)-5-(2-methylbenzo [d]thiazol-6-yl)-7H-pyrrolo [2,3-d]pyrimidin-2-amine [2125];
N-(cis-4-ethoxycyclohexyl)-5-(thieno [3,2-c]pyridin-2-yl)-7H-pyrrolo [2,3-d] pyrimidin-2-amine [2126];
N-(cis-4-ethoxycyclohexyl)-5-(imidazo [1,2-a] pyridin-6-yl)-7H-pyrrolo [2,3-d]pyrimidin-2-amine [2127];
N-(cis-4-ethoxycyclohexyl)-5-(3-fluoroimidazo [1,2-a]pyridin-6-yl)-7H-pyrrolo [2,3-d]pyrimidin-2-amine [2128];
N-(cis-4-ethoxycyclohexyl)-5-(8-fluoroimidazo [1,2-a]pyridin-6-yl)-7H-pyrrolo [2,3-d]pyrimidin-2-amine [2129];
N-(cis-4-ethoxycyclohexyl)-5-(8-fluoro-2-methylimidazo [1,2-a]pyridin-6-yl)-7H-pyrrolo [2,3-d]pyrimidin-2-amine [2130];
N-(cis-4-ethoxycyclohexyl)-5-(8-fluoro-3-methylimidazo [1,2-a]pyridin-6-yl)-7H-pyrrolo [2,3-d]pyrimidin-2-amine [2131];
5-(3-(difluoromethyl)imidazo [1,2-a]pyridin-6-yl)-N-(cis-4-ethoxycyclohexyl)-7H-pyrrolo [2,3-d]pyrimidin-2-amine [2132];
(6-(2-((cis-4-ethoxycyclohexyl)amino)-7H-pyrrolo [2,3-d]pyrimidin-5-yl)-8-fluoroimidazo [1,2-a] pyridin-3-yl)methanol [2133];
N-(2,2-difluoroethyl)-6-(2-((cis-4-ethoxycyclohexyl)amino)-7H-pyrrolo [2,3-d]pyrimidin-5-yl)imidazo[1,2-a]pyridine-3-carboxamide [2134];
N-(cis-4-ethoxycyclohexyl)-5-(imidazo [1,2-a] pyrimidin-6-yl)-7H-pyrrolo [2,3-d]pyrimidin-2-amine [2135];
N-(cis-4-ethoxycyclohexyl)-5-(4-fluoro -1-i sopropyl-2-methyl-1H-benzo [d] imidazol-6-yl)-7H-pyrrolo [2,3-d]pyrimidin-2-amine [2136];
N-(cis-4-ethoxycyclohexyl)-5-(4-fluoro-2-methyl-1-(1-methylpiperidin-4-yl)-1H-benzo[d]imidazol-6-yl)-7H-pyrrolo [2,3-d]pyrimidin-2-amine [2137];
N-(cis-4-ethoxycyclohexyl)-5-(1-i sopropyl-2-methyl-1H-imidazo [4,5-b] pyridin-6-yl)-7H-pyrrolo [2,3-d]pyrimidin-2-amine [2138];
5-(3-(2,2-difluoroethyl)-2-methyl-3H-imidazo[4,5-b]pyridin-5-yl)-N-(cis-4-ethoxycyclohexyl)-7H-pyrrolo [2,3-d]pyrimidin-2-amine [2139];
N-(cis-4-ethoxycyclohexyl)-5-(34 sopropyl-2-methyl-3H-imidazo [4,5-b] pyridin-5-yl)-7H-pyrrolo [2,3-d]pyrimidin-2-amine [2140];
5-([1,2,4]triazolo [1,5-a]pyridin-6-yl)-N-(cis-4-ethoxycyclohexyl)-7H-pyrrolo [2,3-d] pyrimidin-2-amine [2141];
N-(cis-4-ethoxycyclohexyl)-5-(8-fluoro-[1,2,4]triazolo [1,5-a]pyridin-6-yl)-7H-pyrrolo [2,3-d]pyrimidin-2-amine [2142];
N-(cis-4-ethoxycyclohexyl)-5-(1-methyl-1H-benzo[d] [1,2,3]triazol -6-yl)-7H-pyrrolo [2,3-d]pyrimidin-2-amine [2143];
N-(cis-4-ethoxycyclohexyl)-5-(1-ethyl-1H-benzo [d] [1,2,3]triazol-6-yl)-7H-pyrrolo [2,3-d]pyrimidin-2-amine [2144];
N-(cis-4-ethoxycyclohexyl)-5-(imidazo [1,2-b] pyridazin-6-yl)-7H-pyrrolo [2,3-d]pyrimidin-2-amine [2145];
5-(3-chloroimidazo [1,2-b] pyridazin-6-yl)-N-(cis-4-ethoxycyclohexyl)-7H-pyrrolo [2,3-d]pyrimidin-2-amine [2146];
N-(cis-4-ethoxycyclohexyl)-5-(2-methylimidazo [1,2-b]pyridazin-6-yl)-7H-pyrrolo [2,3-d]pyrimidin-2-amine [2147];
N-(cis-4-ethoxycyclohexyl)-5-(3-methylimidazo [1,2-b]pyridazin-6-yl)-7H-pyrrolo [2,3-d]pyrimidin-2-amine [2148];
5-(3-(difluoromethyl)imidazo[1,2-b]pyridazin-6-yl)-N-(cis-4-ethoxycyclohexyl)-7H-pyrrolo [2,3-d]pyrimidin-2-amine [2149];
N-(cis-4-ethoxycyclohexyl)-5-(pyrazolo [1,5-a]pyridin-5-yl)-7H-pyrrolo [2,3-d]pyrimidin-2-amine [2150];
N-(cis-4-ethoxycyclohexyl)-5-(3-(1-methyl-1H-pyrazol-4-yl)pyrazolo [1,5-a]pyridin-5-yl)-7H-pyrrolo [2,3-d]pyrimidin-2-amine [2151];
(5-(2-((cis-4-ethoxycyclohexyl)amino)-7H-pyrrolo [2,3-d]pyrimidin-5-yl)pyrazolo [1,5-a]pyridin-3-yl)(piperidin-1-yl)methanone [2152];
N-(2,2-difluoroethyl)-5-(2-((cis-4-ethoxycyclohexyl)amino)-7H-pyrrolo [2,3-d]pyrimidin-5-yl)pyrazolo [1,5-a]pyridine-3-carboxamide [2153];
5-(2-((cis-4-ethoxycyclohexyl)amino)-7H-pyrrolo [2,3-d]pyrimidin-5-yl)-N-(2-fluoro-2-methylpropyl)pyrazolo [1,5-a]pyridine-3-carboxamide [2154];
5-(2-((cis-4-ethoxycyclohexyl)amino)-7H-pyrrolo [2,3-d]pyrimidin-5-yl)-N-((R)-1,1,1-trifluoropropan-2-yl)pyrazolo [1,5-a]pyridine-3-carboxamide [2155];
N-(3,3-difluorocyclobutyl)-5-(2-((cis-4-ethoxycyclohexyl)amino)-7H-pyrrolo [2,3-d]pyrimidin-5-yl)pyrazolo [1,5-a]pyridine-3-carboxamide [2156];
N-((3,3-difluorocyclobutyl)methyl)-5-(2-((cis-4-ethoxycyclohexyl)amino)-7H-pyrrolo [2,3-d]pyrimidin-5-yl)pyrazolo [1,5-a]pyridine-3-carboxamide [2157];
5-(2-((cis-4-ethoxycyclohexyl)amino)-7H-pyrrolo [2,3-d]pyrimidin-5-yl)-N-((1 r,4r)-4-methoxycyclohexyl)pyrazolo [1,5-a] pyridine-3-carboxamide [2158];
5-(2-((cis-4-ethoxycyclohexyl)amino)-7H-pyrrolo [2,3-d]pyrimidin-5-yl)-N-(1-methylpiperidin-4-yl)pyrazolo [1,5-a]pyridine-3-carboxamide [2159];
5-(2-((cis-4-ethoxycyclohexyl)amino)-7H-pyrrolo [2,3-d]pyrimidin-5-yl)-N-(tetrahydro -2H-pyran-4-yl)pyrazolo [1,5-a] pyridine-3-carboxamide [2160];
5-(2-((cis-4-ethoxycyclohexyl)amino)-7H-pyrrolo [2,3-d]pyrimidin-5-yl)-N-(pyridin-3-yl)pyrazolo [1,5-a]pyridine-3-carboxamide [2161];
N-(cis-4-ethoxycyclohexyl)-5-(pyrazolo [1,5-a]pyrimidin-5-yl)-7H-pyrrolo [2,3-d]pyrimidin-2-amine [2162];
5-(3-cyclopropylpyrazolo [1,5-a] pyrimidin-5-yl)-N-(cis-4-ethoxycyclohexyl)-7H-pyrrolo [2,3-d]pyrimidin-2-amine [2163];
8-(2-((cis-4-ethoxycyclohexyl)amino)-7H-pyrrolo [2,3-d]pyrimidin-5-yl)-3 ,4-dihydrobenzo [f] [1,4]oxazepin-5(2H)-one [2164];
7-(2-((cis-4-ethoxycyclohexyl)amino)-7H-pyrrolo [2,3-d]pyrimidin-5-yl)-2,2-dimethylchroman-4-one [2165];
6-(2-((cis-4-ethoxycyclohexyl)amino)-7H-pyrrolo [2,3-d]pyrimidin-5-yl)-4,4-dimethyl-3 ,4-dihydroisoquinolin-1 (2H)-one [2166];
N-(cis-4-ethoxycyclohexyl)-5-(quinolin-6-yl)-7H-pyrrolo [2,3-d]pyrimidin-2-amine [2167];
N-(cis-4-ethoxycyclohexyl)-5-(quinoxalin-6-yl)-7H-pyrrolo [2,3-d]pyrimidin-2-amine [2168];
N-(cis-4-ethoxycyclohexyl)-5-(quinazolin-6-yl)-7H-pyrrolo [2,3-d]pyrimidin-2-amine [2169];
N-(cis-4-ethoxycyclohexyl)-5-(3-((1-methylpiperidin-4-yl)oxy)quinoxalin-6-yl)-7H-pyrrolo [2,3-d]pyrimidin-2-amine [2170];
5-(imidazo [1,2-b]pyridazin-6-yl)-N-(cis-4-(methoxy-d₃)cyclohexyl)-7H-pyrrolo [2,3-d]pyrimidin-2-amine [2171];
N-(cis-4-(methoxy-d₃)cyclohexyl)-5-(1-methyl-1H-benzo [d][1,2,3]⁻triazol-6-yl)-7H-pyrrolo[2,3-d]pyrimidin-2-amine [2172];
N-(cis-4-(methoxy-d₃)cyclohexyl)-5-(quinoxalin-6-yl)-7H-pyrrolo [2,3-d]pyrimidin-2-amine [2173];
(R)-5-(imidazo[1,2-a]pyridin-6-yl)-4-methoxy-N-(1, 1, 1-trifluoropropan-2-yl)-7H-pyrrolo [2,3-d]pyrimidin-2-amine [2174];
(R)-5-(imidazo[1,2-a]pyridin-6-yl)-4-methoxy-N-(1-methoxypropan-2-yl)-7H-pyrrolo [2,3-d]pyrimidin-2-amine [2175];
N-(cis-4-(2,2-difluoroethoxy)cyclohexyl)-5-(imidazo[1,2-a]pyridin-6-yl)-4-methoxy-7H-pyrrolo[2,3-d]pyrimidin-2-amine [2176];
(R)-4-ethoxy-5-(imidazo[1,2-a]pyridin-6-yl)-N-(1,1,1-trifluoropropan-2-yl)-7H-pyrrolo[2,3-d]pyrimidin-2-amine [2177];
(R)-2-((5-(imidazo[1,2-a]pyridin-6-yl)-2-((1,1,1-trifluoropropan-2-yl)amino)-7H-pyrrolo[2,3-d]pyrimidin-4-ypoxy)ethan-1-ol [2178];
cis-4-((5-(8-fluoroimidazo[1,2-a]pyridin-6-yl)-7H-pyrrolo[2,3-d]pyrimidin-2-yl)amino)-1-methylcyclohexan-1-ol [2179];
5-(8-fluoroimidazo[1,2-a]pyridin-6-yl)-N-(cis-4-(methoxy-d₃)cyclohexyl)-7H-pyrrolo[2,3-d]pyrimidin-2-amine [2180];
N-(cis-4-(2,2-difluoroethoxy)cyclohexyl)-5-(8-fluoroimidazo[1,2-a]pyridin-6-yl)-7H-pyrrolo[2,3-d]pyrimidin-2-amine [2181];
2-((cis-4-((5-(8-fluoroimidazo[1,2-a]pyridin-6-yl)-7H-pyrrolo[2,3-d]pyrimidin-2-yl)amino)cyclohexyl)oxy)ethan-1-ol [2182];
(R)-5-(8-fluoroimidazo[1,2-a]pyridin-6-yl)-4-methoxy-N-(1,1,1-trifluoropropan-2-yl)-7H-pyrrolo[2,3-d]pyrimidin-2-amine [2183];
(R)-5-(8-fluoroimidazo[1,2-a]pyridin-6-yl)-4-methoxy-N-(1-methoxypropan-2-yl)-7H-pyrrolo[2,3-d]pyrimidin-2-amine [2184];
N-(4,4-difluorocyclohexyl)-5-(8-fluoroimidazo[1,2-a]pyridin-6-yl)-4-methoxy-7H-pyrrolo[2,3-d]pyrimidin-2-amine [2185];
cis-4-((5-(8-fluoroimidazo[1,2-a]pyridin-6-yl)-4-methoxy-7H-pyrrolo[2,3-d]pyrimidin-2-yl)amino)-1-methylcyclohexan-1-ol [2186];
N-(cis-4-(2,2-difluoroethoxy)cyclohexyl)-5-(8-fluoroimidazo[1,2-a]pyridin-6-yl)-4-methoxy-7H-pyrrolo[2,3-d]pyrimidin-2-amine [2187];
5-(8-fluoroimidazo[1,2-a]pyridin-6-yl)-4-methoxy-N-(1,4-dioxaspiro[4.5]decan-8-yl)-7H-pyrrolo[2,3-d]pyrimidin-2-amine [2188];
(R)-4-ethoxy-5-(8-fluoroimidazo[1,2-a]pyridin-6-yl)-N-(1,1,1-trifluoropropan-2-yl)-7H-pyrrolo[2,3-d]pyrimidin-2-amine [2189];
cis-4-((4-ethoxy-5-(8-fluoroimidazo[1,2-a]pyridin-6-yl)-7H-pyrrolo[2,3-d]pyrimidin-2-yl)amino)cyclohexan-1-ol [2190];
cis-1-methyl-4-45-(1-methyl-1H-benzo[d][1,2,3]triazol-6-yl)-7H-pyrrolo[2,3-d]pyrimidin-2-yl)amino)cyclohexan-1-ol [2191];
N-(cis-4-(2,2-difluoroethoxy)cyclohexyl)-5-(1-methyl-1H-benzo[d][1,2,3]triazol-6-yl)-7H-pyrrolo[2,3-d]pyrimidin-2-amine [2192];
(R)-4-methoxy-5-(1-methyl-1H-benzo[d][1,2,3]triazol-6-yl)-N-(1,1,1-trifluoropropan-2-yl)-7H-pyrrolo[2,3-d]pyrimidin-2-amine [2193];
(R)-4-methoxy-N-(1-methoxypropan-2-yl)-5-(1-methyl-1H-benzo[d][1,2,3]triazol-6-yl)-7H-pyrrolo[2,3-d]pyrimidin-2-amine [2194];
4-methoxy-5-(1-methyl-1H-benzo[d][1,2,3]triazol-6-yl)-N-(1-methylpiperidin-4-yl)-7H-pyrrolo[2,3-d]pyrimidin-2-amine [2195];
(R)-4-ethoxy-5-(1-methyl-1H-benzo[d][1,2,3]triazol-6-yl)-N-(1,1,1-trifluoropropan-2-yl)-7H-pyrrolo[2,3-d]pyrimidin-2-amine [2196];
cis-4-((4-ethoxy-5-(1-methyl-1H-benzo[d][1,2,3]triazol-6-yl)-7H-pyrrolo[2,3-d]pyrimidin-2-yl)amino)cyclohexan-1-ol [2197];
N-(4,4-difluorocyclohexyl)-5-(1-(2,2-difluoroethyl)-1H-benzo[d][1,2,3]triazol-6-yl)-4-methoxy-7H-pyrrolo[2,3-d]pyrimidin-2-amine [2198];
N-(cis-4-(2,2-difluoroethoxy)cyclohexyl)-5-(imidazo[1,2-b]pyridazin-6-yl)-7H-pyrrolo[2,3-d]pyrimidin-2-amine [2199];
N-(cis-4-(methoxy-d₃)cyclohexyl)-5-(pyrazolo[1,5-a]pyridin-5-yl)-7H-pyrrolo[2,3-d]pyrimidin-2-amine [2200];
N-(cis-4-(2,2-difluoroethoxy)cyclohexyl)-5-(pyrazolo[1,5-a]pyridin-5-yl)-7H-pyrrolo[2,3-d]pyrimidin-2-amine [2201];
2-((cis-4-((5-(pyrazolo[1,5-a]pyridin-5-yl)-7H-pyrrolo[2,3-d]pyrimidin-2-yl)amino)cyclohexyl)oxy)ethan-1-ol [2202];
N-(cis-4-(2-methoxyethoxy)cyclohexyl)-5-(pyrazolo[1,5-a]pyridin-5-yl)-7H-pyrrolo[2,3-d]pyrimidin-2-amine [2203];
(R)-4-methoxy-5-(pyrazolo[1,5-a]pyridin-5-yl)-N-(1,1,1-trifluoropropan-2-yl)-7H-pyrrolo[2,3-d]pyrimidin-2-amine [2204];
(R)-4-methoxy-N-(1-methoxypropan-2-yl)-5-(pyrazolo[1,5-a]pyridin-5-yl)-7H-pyrrolo[2,3-d]pyrimidin-2-amine [2205];
N-(4,4-difluorocyclohexyl)-4-methoxy-5-(pyrazolo[1,5-a]pyridin-5-yl)-7H-pyrrolo[2,3-d]pyrimidin-2-amine [2206];
cis-4-((4-methoxy-5-(pyrazolo[1,5-a]pyridin-5-yl)-7H-pyrrolo[2,3-d]pyrimidin-2-yl)amino)cyclohexan-1-ol [2207];
cis-4-((4-methoxy-5-(pyrazolo[1,5-a]pyridin-5-yl)-7H-pyrrolo[2,3-d]pyrimidin-2-yl)amino)-1-methylcyclohexan-1-ol [2208];
trans-4-44-methoxy-5-(pyrazolo[1,5-a]pyridin-5-yl)-7H-pyrrolo[2,3-d]pyrimidin-2-yl)amino)-1-methylcyclohexan-1-ol [2209];
4-methoxy-N-(cis-4-(methoxy-d₃)cyclohexyl)-5-(pyrazolo [1,5-a] pyridin-5-yl)-7H-pyrrolo [2,3-d]pyrimidin-2-amine [2210];
N-(cis-4-(difluoromethoxy)cyclohexyl)-4-methoxy-5-(pyrazolo [1,5-a]pyridin-5-yl)-7H-pyrrolo [2,3-d]pyrimidin-2-amine [2211];
N-(cis-4-(2,2-difluoroethoxy)cyclohexyl)-4-methoxy-5-(pyrazolo [1,5-a]pyridin-5-yl)-7H-pyrrolo [2,3-d]pyrimidin-2-amine [2212];
2-((cis-4-((4-methoxy-5-(pyrazolo [1,5-a] pyridin-5-yl)-7H-pyrrolo [2,3-d] pyrimidin-2-yl)amino)cyclohexyl)oxy)ethan-1-ol [2213];
4-methoxy-N-(cis-4-(2-methoxyethoxy)cyclohexyl)-5-(pyrazolo [1,5-a] pyridin-5-yl)-7H-pyrrolo [2,3-d]pyrimidin-2-amine [2214];
4-methoxy-5-(pyrazolo [1,5-a]pyridin-5-yl)-N-(1,4-dioxaspiro [4.5] de can-8-yl)-7H-pyrrolo [2,3-d]pyrimidin-2-amine [2215];
4-methoxy-N-(1-methylpiperidin-4-yl)-5-(pyrazolo [1,5-a]pyridin-5-yl)-7H-pyrrolo [2,3-d]pyrimidin-2-amine [2216];
4-methoxy-5-(pyrazolo [1,5-a]pyridin-5-yl)-N-(tetrahydro -2H-pyran-4-yl)-7H-pyrrolo [2,3-d]pyrimidin-2-amine [2217];
(R)-4-ethoxy-5-(pyrazolo [1,5-a]pyridin-5-yl)-N-(1,1,1-trifluoropropan-2-yl)-7H-pyrrolo [2,3-d]pyrimidin-2-amine [2218];
cis-4-((4-ethoxy-5-(pyrazolo [1,5-a] pyridin-5-yl)-7H-pyrrolo [2,3-d]pyrimidin-2-yl)amino)cyclohexan-1-01 [2219];
cis-4-((4-i sop ropoxy-5-(pyrazolo [1,5-a]pyridin-5-yl)-7H-pyrrolo [2,3-d]pyrimidin-2-yl)amino)-1-methylcyclohexan-1-ol [2220];
N-(cis-4-(methoxy-d₃)cyclohexyl)-5-(pyrazolo [1,5-a] pyrimidin-5-yl)-7H-pyrrolo [2,3-d]pyrimidin-2-amine [2221];
N-(cis-4-(2,2-difluoroethoxy)cyclohexyl)-5-(pyrazolo [1,5-a]pyrimidin-5-yl)-7H-pyrrolo [2,3-d]pyrimidin-2-amine [2222];
N-(4,4-difluorocyclohexyl)-4-methoxy-5-(pyrazolo [1,5-a] pyrimidin-5-yl)-7H-pyrrolo [2,3-d]pyrimidin-2-amine [2223];
4-methoxy-5-(pyrazolo [1,5-a]pyrimidin-5-yl)-N-(tetrahydro-2H-pyran-4-yl)-7H-pyrrolo [2,3-d]pyrimidin-2-amine [2224];
cis-4-((4-ethoxy-5-(pyrazolo [1,5-a] pyrimidin-5-yl)-7H-pyrrolo [2,3-d]pyrimidin-2-yl)amino)cyclohexan-1-01 [2225];
N-(cis-4-(2-methoxyethoxy)cyclohexyl)-5-(quinolin-6-yl)-7H-pyrrolo [2,3-d] pyrimidin-2-amine [2226];
N-(4,4-difluorocyclohexyl)-4-methoxy-5-(quinolin-6-yl)-7H-pyrrolo [2,3-d]pyrimidin-2-amine [2227];
cis-1-methyl-4-((5-(quinoxalin-6-yl)-7H-pyrrolo [2,3-d]pyrimidin-2-yl)amino)cyclohexan-1-ol [2229];
2-((cis-4-((5-(quinoxalin-6-yl)-7H-pyrrolo [2,3-d]pyrimidin-2-yl)amino)cyclohexyl)oxy)ethan-1-ol [2229];
N-(4,4-difluorocyclohexyl)-4-methoxy-5-(quinoxalin-6-yl)-7H-pyrrolo [2,3-d]pyrimidin-2-amine [2230];
cis-4-((4-methoxy-5-(quinoxalin-6-yl)-7H-pyrrolo [2,3-d] pyrimidin-2-yl)amino)-1-methylcyclohexan-1-ol [2231];
cis-4-((4-ethoxy-5-(quinoxalin-6-yl)-7H-pyrrolo [2,3-d]pyrimidin-2-yl)amino)cyclohexan-1-ol [2232];
cis-4-((5-(imidazo [1,2-a] pyridin-6-yl)-4-methoxy-7H-pyrrolo [2,3-d]pyrimidin-2-yl)amino)-1-methylcyclohexan-1-ol [2233];
cis-1-methyl-4-((5-(pyrazolo [1,5-a]pyridin-5-yl)-7H-pyrrolo [2,3-d]pyrimidin-2-yl)amino)cyclohexan-1-01 [2234];
cis-4-((5-(imidazo [1,2-a] pyridin-6-yl)-7H-pyrrolo [2,3-d] pyrimidin-2-yl)amino)-1-methylcyclohexan-1-ol [2235];
trans-4-((5-(imidazo [1,2-a]pyridin-6-yl)-7H-pyrrolo [2,3-d]pyrimidin-2-yl)amino)-1-methylcyclohexan-1-ol [2236];
trans-4-45-(8-fluoroimidazo [1,2-a]pyridin-6-yl)-7H-pyrrolo [2,3-d]pyrimidin-2-yl)amino)-1-methylcyclohexan-1-ol [2237];
trans-1-methyl-4-((5-(pyrazolo [1,5-a]pyridin-5-yl)-7H-pyrrolo [2,3-d]pyrimidin-2-yl)amino)cyclohexan-1-01 [2238];
cis-4-45-(3-(2,2-difluoroethyl)-2-methyl-3H-imidazo [4,5-b]pyridin-5-yl)-7H-pyrrolo [2,3-d]pyrimidin-2-yl)amino)-1-methylcyclohexan-1-ol [2239];
N-(2-fluoro-2-methylpropyl)-5-(imidazo [1,2-a]pyridin-6-yl)-4-methoxy-7H-pyrrolo [2,3-d]pyrimidin-2-amine [2240];
N-(4,4-difluorocyclohexyl)-5-(imidazo[1,2-a]pyridin-6-yl)-4-methoxy-7H-pyrrolo [2,3-d]pyrimidin-2-amine [2241];
5-(imidazo [1,2-a] pyridin-6-yl)-4-methoxy-N-(cis-4-(methoxy-d₃)cyclohexyl)-7H-pyrrolo [2,3-d]pyrimidin-2-amine [2242];
5-(imidazo [1,2-a] pyridin-6-yl)-4-methoxy-N-(trans-4-(methoxy-d₃)cyclohexyl)-7H-pyrrolo [2,3-d]pyrimidin-2-amine [2243];
N-(cis-4-(difluoromethoxy)cyclohexyl)-5-(imidazo [1,2-a]pyridin-6-yl)-4-methoxy-7H-pyrrolo [2,3-d]pyrimidin-2-amine [2244];
N-(trans-4-(difluoromethoxy)cyclohexyl)-5-(imidazo [1,2-a]pyridin-6-yl)-4-methoxy-7H-pyrrolo [2,3-d]pyrimidin-2-amine [2245];
5-(imidazo [1,2-a]pyridin-6-yl)-4-methoxy-N-(cis-4-(trifluoromethoxy)cyclohexyl)-7H-pyrrolo [2,3-d]pyrimidin-2-amine [2246];
trans-4-((5-(imidazo [1,2-a]pyridin-6-yl)-4-methoxy-7H-pyrrolo [2,3-d]pyrimidin-2-yl)amino)-1-methylcyclohexan-1-ol [2247];
5-(imidazo [1,2-a]pyridin-6-yl)-4-methoxy-N-(cis-4-methoxy-4-methylcyclohexyl)-7H-pyrrolo [2,3-d]pyrimidin-2-amine [2248];
trans-1-ethyl-4-((5-(imidazo [1,2-a]pyridin-6-yl)-4-methoxy-7H-pyrrolo [2,3-d]pyrimidin-2-yl)amino)cyclohexan-1-ol [2249];
5-(imidazo [1,2-a]pyridin-6-yl)-4-methoxy-N-(1,5-dioxaspiro [5 .5]undecan-9-yl)-7H-pyrrolo [2,3-d]pyrimidin-2-amine [2250];
N-(5,12-dioxadispiro [2.2.56.23]tridecan-9-yl)-5-(imidazo [1,2-a]pyridin-6-yl)-4-methoxy-7H-pyrrolo [2,3-d]pyrimidin-2-amine [2251];
N-((3S,4R)-3-fluorotetrahydro-2H-pyran-4-yl)-5-(imidazo [1,2-a]pyridin-6-yl)-4-methoxy-7H-pyrrolo [2,3-d]pyrimidin-2-amine [2252];
N-((3R,4R)-3-fluorotetrahydro-2H-pyran-4-yl)-5-(imidazo [1,2-a]pyridin-6-yl)-4-methoxy-7H-pyrrolo [2,3-d]pyrimidin-2-amine [2253];
cis-4-((4-ethoxy-5-(imidazo [1,2-a] pyridin-6-yl)-7H-pyrrolo [2,3-d]pyrimidin-2-yl)amino)-1-methylcyclohexan-1-ol [2254];
N-(2-fluoro-2-methylpropyl)-5-(8-fluoroimidazo [1,2-a]pyridin-6-yl)-4-methoxy-7H-pyrrolo [2,3-d]pyrimidin-2-amine [2255];
5-(8-fluoroimidazo [1,2-a]pyridin-6-yl)-4-methoxy-N-(cis-4-(methoxy-d₃)cyclohexyl)-7H-pyrrolo [2,3-d]pyrimidin-2-amine [2256];
5-(8-fluoroimidazo [1,2-a]pyridin-6-yl)-4-methoxy-N-(trans-4-(methoxy-d₃)cyclohexyl)-7H-pyrrolo [2,3-d]pyrimidin-2-amine [2257];
N-(cis-4-(difluoromethoxy)cyclohexyl)-5-(8-fluoroimidazo [1,2-a]pyridin-6-yl)-4-methoxy-7H-pyrrolo [2,3-d]pyrimidin-2-amine [2258];
N-(trans-4-(difluoromethoxy)cyclohexyl)-5-(8-fluoroimidazo [1,2-a]pyridin-6-yl)-4-methoxy-7H-pyrrolo [2,3-d]pyrimidin-2-amine [2259];
5-(8-fluoroimidazo [1,2-a]pyridin-6-yl)-4-methoxy-N-(cis-4-(trifluoromethoxy)cyclohexyl)-7H-pyrrolo [2,3-d]pyrimidin-2-amine [2260];
trans-44(5-(8-fluoroimidazo [1,2-a]pyridin-6-yl)-4-methoxy-7H-pyrrolo [2,3-d]pyrimidin-2-yl)amino)-1-methylcyclohexan-1-ol [2261];
5-(8-fluoroimidazo [1,2-a]pyridin-6-yl)-4-methoxy-N-(cis-4-methoxy-4-methylcyclohexyl)-7H-pyrrolo [2,3-d]pyrimidin-2-amine [2262];
trans-1-ethyl-4-((5-(8-fluoroimidazo[1,2-a]pyridin-6-yl)-4-methoxy-7H-pyrrolo [2,3-d]pyrimidin-2-yl)amino)cyclohexan-1-ol [2263];
5-(8-fluoroimidazo [1,2-a]pyridin-6-yl)-4-methoxy-N4 1,5-dioxaspiro [5 .5]undecan-9-yl)-7H-pyrrolo [2,3-d]pyrimidin-2-amine [2264];
N-(5, 12-dioxadispiro [2.2.5 6 .23]tridecan-9-yl)-5-(8-fluoroimidazo [1,2-a]pyridin-6-yl)-4-methoxy-7H-pyrrolo [2,3-d]pyrimidin-2-amine [2265];
5-(8-fluoroimidazo [1,2-a]pyridin-6-yl)-N-((3 S,4R)-3-fluorotetrahydro-2H-pyran-4-yl)-4-methoxy-7H-pyrrolo [2,3-d]pyrimidin-2-amine [2266];
5-(8-fluoroimidazo [1,2-a]pyridin-6-yl)-N-((3R,4R)-3-fluorotetrahydro-2H-pyran-4-yl)-4-methoxy-7H-pyrrolo [2,3-d]pyrimidin-2-amine [2267];
N-(4,4-difluorocyclohexyl)-5-(imidazo[1,2-a]pyrimidin-6-yl)-4-methoxy-7H-pyrrolo [2,3-d]pyrimidin-2-amine [2268];
5-(1-(2,2-difluoroethyl)-2-methyl-1H-imidazo [4,5-b]pyridin-6-yl)-N-(2-fluoro-2-methylpropyl)-4-methoxy-7H-pyrrolo [2,3-d]pyrimidin-2-amine [2269];
(R)-5-(1-(2,2-difluoroethyl)-2-methyl-1H-imidazo [4,5-b]pyridin-6-yl)-4-methoxy-N-(1, 1, 1-trifluoropropan-2-yl)-7H-pyrrolo [2,3-d]pyrimidin-2-amine [2270];
(R)-5-(1-(2,2-difluoroethyl)-2-methyl-1H-imidazo [4,5-b]pyridin-6-yl)-4-methoxy-N-(1-methoxypropan-2-yl)-7H-pyrrolo [2,3-d]pyrimidin-2-amine [2271];
N-(4,4-difluorocyclohexyl)-5-(1-(2,2-difluoroethyl)-2-methyl-1H-imidazo [4,5-b]pyridin-6-yl)-4-methoxy-7H-pyrrolo [2,3-d]pyrimidin-2-amine [2272];
5-(1-(2,2-difluoroethyl)-2-methyl-1H-imidazo [4,5-b]pyridin-6-yl)-4-methoxy-N-(cis-4-(methoxy-d₃)cyclohexyl)-7H-pyrrolo [2,3-d] pyrimidin-2-amine [2273];
5-(1-(2,2-difluoroethyl)-2-methyl-1H-imidazo [4,5-b]pyridin-6-yl)-N-(cis-4-(difluoromethoxy)cyclohexyl)-4-methoxy-7H-pyrrolo [2,3-d]pyrimidin-2-amine [2274];
5-(1-(2,2-difluoroethyl)-2-methyl-1H-imidazo [4,5-b]pyridin-6-yl)-4-methoxy-N-(cis-4-(trifluoromethoxy)cyclohexyl)-7H-pyrrolo [2,3-d] pyrimidin-2-amine [2275];
5-(3-(2,2-difluoroethyl)-2-methyl-3H-imidazo [4,5-b]pyridin-5-yl)-N-(2-fluoro-2-methylpropyl)-4-methoxy-7H-pyrrolo [2,3-d]pyrimidin-2-amine [2276];
(R)-5-(3-(2,2-difluoroethyl)-2-methyl-3H-imidazo [4,5-b]pyridin-5-yl)-4-methoxy-N-(1, 1, 1-trifluoropropan-2-yl)-7H-pyrrolo [2,3-d]pyrimidin-2-amine [2277];
(R)-5-(3-(2,2-difluoroethyl)-2-methyl-3H-imidazo[4,5-b]pyridin-5-yl)-4-methoxy-N-(1-methoxypropan-2-yl)-7H-pyrrolo[2,3-d]pyrimidin-2-amine [2278];
N-(4,4-difluorocyclohexyl)-5-(3-(2,2-difluoroethyl)-2-methyl-3H-imidazo[4,5-b]pyridin-5-yl)-4-methoxy-7H-pyrrolo[2,3-d]pyrimidin-2-amine [2279];
5-(3-(2,2-difluoroethyl)-2-methyl-3H-imidazo[4,5-b]pyridin-5-yl)-4-methoxy-N-(cis-4-(methoxy-d₃)cyclohexyl)-7H-pyrrolo[2,3-d]pyrimidin-2-amine [2280];
5-(3-(2,2-difluoroethyl)-2-methyl-3H-imidazo[4,5-b]pyridin-5-yl)-N-(cis-4-(difluoromethoxy)cyclohexyl)-4-methoxy-7H-pyrrolo[2,3-d]pyrimidin-2-amine [2281];
5-(3-(2,2-difluoroethyl)-2-methyl-3H-imidazo[4,5-b]pyridin-5-yl)-4-methoxy-N-(cis-4-(trifluoromethoxy)cyclohexyl)-7H-pyrrolo[2,3-d]pyrimidin-2-amine [2282];
N-(2-fluoro-2-methylpropyl)-4-methoxy-5-(1-methyl-1H-benzo[d][1,2,3]triazol-6-yl)-7H-pyrrolo[2,3-d]pyrimidin-2-amine [2283];
N-(4,4-difluorocyclohexyl)-4-methoxy-5-(1-methyl-1H-benzo[d][1,2,3]triazol-6-yl)-7H-pyrrolo[2,3-d]pyrimidin-2-amine [2284];
4-methoxy-N-(cis-4-(methoxy-d₃)cyclohexyl)-5-(1-methyl-1H-benzo[d][1,2,3]triazol-6-yl)-7H-pyrrolo[2,3-d]pyrimidin-2-amine [2285];
4-methoxy-N-(trans-4-(methoxy-d₃)cyclohexyl)-5-(1-methyl-1H-benzo[d][1,2,3]triazol-6-yl)-7H-pyrrolo[2,3-d]pyrimidin-2-amine [2286];
N-(cis-4-(difluoromethoxy)cyclohexyl)-4-methoxy-5-(1-methyl-1H-benzo[d][1,2,3]triazol-6-yl)-7H-pyrrolo[2,3-d]pyrimidin-2-amine [2287];
N-(trans-4-(difluoromethoxy)cyclohexyl)-4-methoxy-5-(1-methyl-1H-benzo[d][1,2,3]triazol-6-yl)-7H-pyrrolo[2,3-d]pyrimidin-2-amine [2288];
4-methoxy-5-(1-methyl-1H-benzo[d][1,2,3]triazol-6-yl)-N-(cis-4-(trifluoromethoxy)cyclohexyl)-7H-pyrrolo[2,3-d]pyrimidin-2-amine [2289];
4-methoxy-5-(1-methyl-1H-benzo[d][1,2,3]triazol-6-yl)-N-(1,5-dioxaspiro[5.5]undecan-9-yl)-7H-pyrrolo[2,3-d]pyrimidin-2-amine [2290];
N-(5,12-dioxadispiro[2.2.56.23]tridecan-9-yl)-4-methoxy-5-(1-methyl-1H-benzo[d][1,2,3]triazol-6-yl)-7H-pyrrolo[2,3-d]pyrimidin-2-amine [2291];
5-(1-(2,2-difluoroethyl)-1H-benzo[d][1,2,3]triazol-6-yl)-N-(2-fluoro-2-methylpropyl)-4-methoxy-7H-pyrrolo[2,3-d]pyrimidin-2-amine [2292];
(R)-5-(1-(2,2-difluoroethyl)-1H-benzo[d][1,2,3]triazol-6-yl)-4-methoxy-N-(1,1,1-trifluoropropan-2-yl)-7H-pyrrolo[2,3-d]pyrimidin-2-amine [2293];
(R)-5-(1-(2,2-difluoroethyl)-1H-benzo[d][1,2,3]triazol-6-yl)-4-methoxy-N-(1-methoxypropan-2-yl)-7H-pyrrolo[2,3-d]pyrimidin-2-amine [2294];
5-(1-(2,2-difluoroethyl)-1H-benzo [d] [1,2,3]triazol-6-yl)-4-methoxy-N-(cis-4-(methoxy-d₃)cyclohexyl)-7H-pyrrolo [2,3-d] pyrimidin-2-amine [2295];
5-(1-(2,2-difluoroethyl)-1H-benzo [d] [1,2,3]triazol-6-yl)-N-(cis-4-(difluoromethoxy)cyclohexyl)-4-methoxy-7H-pyrrolo [2,3-d]pyrimidin-2-amine [2296];
5-(1-(2,2-difluoroethyl)-1H-benzo [d] [1,2,3]triazol-6-yl)-4-methoxy-N-(cis-4-(trifluoromethoxy)cyclohexyl)-7H-pyrrolo [2,3-d] pyrimidin-2-amine [2297];
N-(2-fluoro-2-methylpropyl)-5-(imidazo [1,2-b]pyridazin-6-yl)-4-methoxy-7H-pyrrolo [2,3-d]pyrimidin-2-amine [2298];
(R)-5-(imidazo [1,2-b] pyridazin-6-yl)-4-methoxy-N-(1,1,1-trifluoropropan-2-yl)-7H-pyrrolo [2,3-d]pyrimidin-2-amine [2299];
(R)-5-(imidazo [1,2-b] pyridazin-6-yl)-4-methoxy-N-(1-methoxypropan-2-yl)-7H-pyrrolo [2,3-d]pyrimidin-2-amine [2300];
N-(4,4-difluorocyclohexyl)-5-(imidazo [1,2-b] pyridazin-6-yl)-4-methoxy-7H-pyrrolo [2,3-d]pyrimidin-2-amine [2301];
5-(imidazo [1,2-b]pyridazin-6-yl)-4-methoxy-N-(cis-4-(methoxy-d₃)cyclohexyl)-7H-pyrrolo [2,3-d]pyrimidin-2-amine [2302];
N-(cis-4-(difluoromethoxy)cyclohexyl)-5-(imidazo [1,2-b] pyridazin-6-yl)-4-methoxy-7H-pyrrolo [2,3-d]pyrimidin-2-amine [2303];
5-(imidazo [1,2-b]pyridazin-6-yl)-4-methoxy-N-(cis-4-(trifluoromethoxy)cyclohexyl)-7H-pyrrolo [2,3-d]pyrimidin-2-amine [2304];
N-(2-fluoro-2-methylpropyl)-4-methoxy-5-(pyrazolo [1,5-a]pyridin-5-yl)-7H-pyrrolo [2,3-d]pyrimidin-2-amine [2305];
4-methoxy-N-(cis-4-methoxycyclohexyl)-5-(pyrazolo [1,5-a]pyridin-5-yl)-7H-pyrrolo [2,3-d]pyrimidin-2-amine [2306];
4-methoxy-N-(trans-4-(methoxy-d₃)cyclohexyl)-5-(pyrazolo [1,5-a]pyridin-5-yl)-7H-pyrrolo [2,3-d]pyrimidin-2-amine [2307];
N-(trans-4-(difluoromethoxy)cyclohexyl)-4-methoxy-5-(pyrazolo [1,5-a]pyridin-5-yl)-7H-pyrrolo [2,3-d]pyrimidin-2-amine [2308];
4-methoxy-5-(pyrazolo [1,5-a]pyridin-5-yl)-N-(cis-4-(trifluoromethoxy)cyclohexyl)-7H-pyrrolo [2,3-d]pyrimidin-2-amine [2309];
4-methoxy-N-(cis-4-methoxy-4-methylcyclohexyl)-5-(pyrazolo [1,5-a]pyridin-5-yl)-7H-pyrrolo [2,3-d]pyrimidin-2-amine [2310];
trans-1-ethyl-4-44-methoxy-5-(pyrazolo [1,5-a]pyridin-5-yl)-7H-pyrrolo [2,3-d] pyrimidin-2-yl)amino)cyclohexan-1-01 P3111;
4-methoxy-5-(pyrazolo[1,5-a]pyridin-5-yl)-N-(1,5-dioxaspiro[5.5]undecan-9-yl)-7H-pyrrolo[2,3-d]pyrimidin-2-amine [2312];
N-(5,12-dioxadispiro[2.2.56.23]tridecan-9-yl)-4-methoxy-5-(pyrazolo[1,5-a]pyridin-5-yl)-7H-pyrrolo[2,3-d]pyrimidin-2-amine [2313];
(R)-4-methoxy-5-(pyrazolo[1,5-a]pyridin-5-yl)-N-(tetrahydro-2H-pyran-3-yl)-7H-pyrrolo[2,3-d]pyrimidin-2-amine [2314];
(S)-4-methoxy-5-(pyrazolo[1,5-a]pyridin-5-yl)-N-(tetrahydro-2H-pyran-3-yl)-7H-pyrrolo[2,3-d]pyrimidin-2-amine [2315];
N-((3S,4R)-3-fluorotetrahydro-2H-pyran-4-yl)-4-methoxy-5-(pyrazolo[1,5-a]pyridin-5-yl)-7H-pyrrolo[2,3-d]pyrimidin-2-amine [2316];
N-((3R,4R)-3-fluorotetrahydro-2H-pyran-4-yl)-4-methoxy-5-(pyrazolo[1,5-a]pyridin-5-yl)-7H-pyrrolo[2,3-d]pyrimidin-2-amine [2317];
(3S,4R)-4-((4-methoxy-5-(pyrazolo[1,5-a]pyridin-5-yl)-7H-pyrrolo[2,3-d]pyrimidin-2-yl)amino)tetrahydro-2H-pyran-3-ol [2318];
4-methoxy-N-((3S,4R)-3-methoxytetrahydro-2H-pyran-4-yl)-5-(pyrazolo[1,5-a]pyridin-5-yl)-7H-pyrrolo[2,3-d]pyrimidin-2-amine [2319];
cis-4-((4-isopropoxy-5-(pyrazolo[1,5-a]pyridin-5-yl)-7H-pyrrolo[2,3-d]pyrimidin-2-yl)amino)-1-methylcyclohexan-1-ol [2320];
(R)-4-methoxy-5-(pyrazolo[1,5-a]pyridin-5-yl)-N-(tetrahydrofuran-3-yl)-7H-pyrrolo[2,3-d]pyrimidin-2-amine [2321];
(S)-4-methoxy-5-(pyrazolo[1,5-a]pyridin-5-yl)-N-(tetrahydrofuran-3-yl)-7H-pyrrolo[2,3-d]pyrimidin-2-amine [2322];
N-((1R,5S,6r)-3-oxabicyclo[3.1.0]hexan-6-yl)-4-methoxy-5-(pyrazolo[1,5-a]pyridin-5-yl)-7H-pyrrolo[2,3-d]pyrimidin-2-amine [2323];
N-((1R,5S,6s)-3-oxabicyclo[3.1.0]hexan-6-yl)-4-methoxy-5-(pyrazolo[1,5-a]pyridin-5-yl)-7H-pyrrolo[2,3-d]pyrimidin-2-amine [2324];
N-(2-fluoro-2-methylpropyl)-4-methoxy-5-(pyrazolo[1,5-a]pyrimidin-5-yl)-7H-pyrrolo[2,3-d]pyrimidin-2-amine [2325];
(R)-4-methoxy-5-(pyrazolo[1,5-a]pyrimidin-5-yl)-N-(1,1,1-trifluoropropan-2-yl)-7H-pyrrolo[2,3-d]pyrimidin-2-amine [2326];
(R)-4-methoxy-N-(1-methoxypropan-2-yl)-5-(pyrazolo[1,5-a]pyrimidin-5-yl)-7H-pyrrolo[2,3-d]pyrimidin-2-amine [2327];
4-methoxy-N-(cis-4-(methoxy-d₃)cyclohexyl)-5-(pyrazolo[1,5-a]pyrimidin-5-yl)-7H-pyrrolo[2,3-d]pyrimidin-2-amine [2328];
N-(cis-4-(difluoromethoxy)cyclohexyl)-4-methoxy-5-(pyrazolo [1,5-a]pyrimidin-5-yl)-7H-pyrrolo [2,3-d]pyrimidin-2-amine [2329];
4-methoxy-5-(pyrazolo [1,5-a]pyrimidin-5-yl)-N-(cis-4-(trifluoromethoxy)cyclohexyl)-7H-pyrrolo [2,3-d]pyrimidin-2-amine [2330];
(1s,3s)-3-((5-(imidazo [1,2-a]pyridin-6-yl)-7H-pyrrolo [2,3-d] pyrimidin-2-yl)amino)-N,N,1-trimethylcyclobutane-1-carboxamide [2331];
(1r,3r)-3-((5-(imidazo [1,2-a]pyridin-6-yl)-7H-pyrrolo [2,3-d]pyrimidin-2-yl)amino)-N,N,1-trimethylcyclobutane-1-carboxamide [2332];
((1r,3r)-3-((5-(imidazo [1,2-a]pyridin-6-yl)-7H-pyrrolo [2,3-d] pyrimidin-2-yl)amino)-1-methylcyclobutyl)(pyrrolidin-1-yl)methanone [2333];
((1s,3s)-3-((5-(imidazo [1,2-a]pyridin-6-yl)-7H-pyrrolo [2,3-d] pyrimidin-2-yl)amino)-1-methylcyclobutyl)(pyrrolidin-1-yl)methanone [2334];
N-((1r,3r)-3-((5-(imidazo [1,2-a] pyridin-6-yl)-7H-pyrrolo [2,3-d] pyrimidin-2-yl)amino)-1-methylcyclobutyl)acetamide [2335];
N-((1s,3s)-3-((5-(imidazo [1,2-a] pyridin-6-yl)-7H-pyrrolo [2,3-d] pyrimidin-2-yl)amino)-1-methylcyclobutypacetamide [2336];
N-((1r,3r)-3-((5-(imidazo [1,2-a] pyridin-6-yl)-7H-pyrrolo [2,3-d] pyrimidin-2-yl)amino)-1-methylcyclobutyl)propionamide [2337];
N-((1s,3s)-3-((5-(imidazo [1,2-a] pyridin-6-yl)-7H-pyrrolo [2,3-d] pyrimidin-2-yl)amino)-1-methylcyclobutyl)propionamide [2338];
1-((1r,3r)-3-((5-(imidazo [1,2-a]pyridin-6-yl)-7H-pyrrolo[2,3-d]pyrimidin-2-yl)amino)-1-methylcyclobutyl)pyrrolidin-2-one [2339];
1-((1s,3s)-3-((5-(imidazo [1,2-a] pyridin-6-yl)-7H-pyrrolo [2,3-d] pyrimidin-2-yl)amino)-1-methylcyclobutyl)pyrrolidin-2-one [2340];
(1s,3s)-3-((5-(imidazo [1,2-a]pyridin-6-yl)-7H-pyrrolo [2,3-d] pyrimidin-2-yl)amino)-1-methylcyclobutan-1-ol [2341];
(1r,3r)-3-((5-(imidazo [1,2-a]pyridin-6-yl)-7H-pyrrolo [2,3-d]pyrimidin-2-yl)amino)-1-methylcyclobutan-1-ol [2342];
(1s,4s)-4-((5-(imidazo [1,2-a]pyridin-6-yl)-7H-pyrrolo [2,3-d] pyrimidin-2-yl)amino)-N,N-dimethylcyclohexane-1-carboxamide [2343];
((1s,4s)-4-((5-(imidazo [1,2-a]pyridin-6-yl)-7H-pyrrolo [2,3-d] pyrimidin-2-yl)amino)cyclohexyl)(pyrrolidin-1-yl)methanone [2344];
N-((1s,4s)-4-((5-(imidazo [1,2-a] pyridin-6-yl)-7H-pyrrolo [2,3-d] pyrimidin-2-yl)amino)cyclohexyl)acetamide [2345];
N-((1r,4r)-4-((5-(imidazo[1,2-a]pyridin-6-yl)-7H-pyrrolo[2,3-d]pyrimidin-2-yl)amino)cyclohexypacetamide [2346];
1-((1s,4s)-4-((5-(imidazo[1,2-a]pyridin-6-yl)-7H-pyrrolo[2,3-d]pyrimidin-2-yl)amino)cyclohexyl)pyrrolidin-2-one [2347];
N-((1r,40-4-45-(imidazo[1,2-a]pyridin-6-yl)-7H-pyrrolo[2,3-d]pyrimidin-2-yl)amino)-1-methylcyclohexyl)acetamide [2348];
(1s,4s)-4-((5-(imidazo[1,2-a]pyridin-6-yl)-7H-pyrrolo[2,3-d]pyrimidin-2-yl)amino)-1-methylcyclohexan-1-ol [2349];
(1s,4s)-1-ethyl-4-((5-(imidazo[1,2-a]pyridin-6-yl)-7H-pyrrolo[2,3-d]pyrimidin-2-yl)amino)cyclohexan-1-ol [2350];
1-(4-((5-(imidazo[1,2-a]pyridin-6-yl)-7H-pyrrolo[2,3-d]pyrimidin-2-yl)amino)piperidin-1-yl)ethan-1-one [2351];
(S)-5-((5-(imidazo[1,2-a]pyridin-6-yl)-7H-pyrrolo[2,3-d]pyrimidin-2-yl)amino)-1-methylpiperidin-2-one [2352];
(R)-5-((5-(imidazo[1,2-a]pyridin-6-yl)-7H-pyrrolo[2,3-d]pyrimidin-2-yl)amino)-1-methylpiperidin-2-one [2353];
1-(2-((5-(imidazo[1,2-a]pyridin-6-yl)-7H-pyrrolo[2,3-d]pyrimidin-2-yl)amino)-7-azaspiro[3.5]nonan-7-yl)ethan-1-one [2354];
1-((3aR,5s,6aS)-5-((5-(imidazo[1,2-a]pyridin-6-yl)-7H-pyrrolo[2,3-d]pyrimidin-2-yl)amino)hexahydrocyclopenta[c]pyrrol-2(1H)-yl)ethan-1-one [2355];
1-((3aR,5r,6aS)-5-((5-(imidazo[1,2-a]pyridin-6-yl)-7H-pyrrolo[2,3-d]pyrimidin-2-yl)amino)hexahydrocyclopenta[c]pyrrol-2(1H)-yl)ethan-1-one [2356];
1-(7-((5-(imidazo[1,2-a]pyridin-6-yl)-7H-pyrrolo[2,3-d]pyrimidin-2-yl)amino)-2-azaspiro[3.5]nonan-2-yl)ethan-1-one [2357];
5-(imidazo[1,2-a]pyridin-6-yl)-N-(2-oxaspiro[3.5]nonan-7-yl)-7H-pyrrolo[2,3-d]pyrimidin-2-amine [2358];
5-(imidazo[1,2-a]pyridin-6-yl)-N-(1,4-dioxaspiro[4.5]decan-8-yl)-7H-pyrrolo[2,3-d]pyrimidin-2-amine [2359];
(1s,3s)-3-((5-(imidazo[1,2-a]pyridin-6-yl)-4-methoxy-7H-pyrrolo[2,3-d]pyrimidin-2-yl)amino)-N,N,1-trimethylcyclobutane-1-carboxamide [2360];
(1r,3r)-3-((5-(imidazo[1,2-a]pyridin-6-yl)-4-methoxy-7H-pyrrolo[2,3-d]pyrimidin-2-yl)amino)-N,N,1-trimethylcyclobutane-1-carboxamide [2361];
((1s,3s)-3-((5-(imidazo[1,2-a]pyridin-6-yl)-4-methoxy-7H-pyrrolo[2,3-d]pyrimidin-2-yl)amino)-1-methylcyclobutyl)(pyrrolidin-1-yl)methanone [2362];
((1r,3r)-3-((5-(imidazo[1,2-a]pyridin-6-yl)-4-methoxy-7H-pyrrolo[2,3-d]pyrimidin-2-yl)amino)-1-methylcyclobutyl)(pyrrolidin-1-yl)methanone [2363];
N-((1r,3r)-3-((5-(imidazo[1,2-a]pyridin-6-yl)-4-methoxy-7H-pyrrolo[2,3-d]pyrimidin-2-yl)amino)-1-methylcyclobutypacetamide [2364];
N-((1s,3s)-3-((5-(imidazo[1,2-a]pyridin-6-yl)-4-methoxy-7H-pyrrolo[2,3-d]pyrimidin-2-yl)amino)-1-methylcyclobutypacetamide [2365];
N-((1s,3s)-3-((5-(imidazo[1,2-a]pyridin-6-yl)-4-methoxy-7H-pyrrolo[2,3-d]pyrimidin-2-yl)amino)-1-methylcyclobutyl)propionamide [2366];
N-((1r,3r)-3-((5-(imidazo[1,2-a]pyridin-6-yl)-4-methoxy-7H-pyrrolo[2,3-d]pyrimidin-2-yl)amino)-1-methylcyclobutyl)propionamide [2367];
1-((1r,3r)-3-((5-(imidazo[1,2-a]pyridin-6-yl)-4-methoxy-7H-pyrrolo[2,3-d]pyrimidin-2-yl)amino)-1-methylcyclobutyppyrrolidin-2-one [2368];
1-((1s,3s)-3-((5-(imidazo[1,2-a]pyridin-6-yl)-4-methoxy-7H-pyrrolo[2,3-d]pyrimidin-2-yl)amino)-1-methylcyclobutyppyrrolidin-2-one [2369];
(1r,3r)-3-((5-(imidazo[1,2-a]pyridin-6-yl)-4-methoxy-7H-pyrrolo[2,3-d]pyrimidin-2-yl)amino)-1-methylcyclobutan-1-ol [2370];
(1s,3s)-3-((5-(imidazo[1,2-a]pyridin-6-yl)-4-methoxy-7H-pyrrolo[2,3-d]pyrimidin-2-yl)amino)-1-methylcyclobutan-1-ol [2371];
(1s,4s)-4-((5-(imidazo[1,2-a]pyridin-6-yl)-4-methoxy-7H-pyrrolo[2,3-d]pyrimidin-2-yl)amino)-N,N-dimethylcyclohexane-1-carboxamide [2372];
((1s,4s)-4-((5-(imidazo[1,2-a]pyridin-6-yl)-4-methoxy-7H-pyrrolo[2,3-d]pyrimidin-2-yl)amino)cyclohexyl)(pyrrolidin-1-yl)methanone [2373];
N-((1r,40-4-45-(imidazo[1,2-a]pyridin-6-yl)-4-methoxy-7H-pyrrolo[2,3-d]pyrimidin-2-yl)amino)cyclohexypacetamide [2374];
N-((1s,4s)-4-((5-(imidazo[1,2-a]pyridin-6-yl)-4-methoxy-7H-pyrrolo[2,3-d]pyrimidin-2-yl)amino)cyclohexypacetamide [2375];
1-((1s,4s)-4-((5-(imidazo[1,2-a]pyridin-6-yl)-4-methoxy-7H-pyrrolo[2,3-d]pyrimidin-2-yl)amino)cyclohexyl)pyrrolidin-2-one [2376];
N-((1r,40-4-45-(imidazo[1,2-a]pyridin-6-yl)-4-methoxy-7H-pyrrolo[2,3-d]pyrimidin-2-yl)amino)-1-methylcyclohexyl)acetamide [2377];
(1r,40-4-45-(imidazo[1,2-a]pyridin-6-yl)-4-methoxy-7H-pyrrolo[2,3-d]pyrimidin-2-yl)amino)-1-methylcyclohexan-1-ol [2378];
(1r,40-4-45-(imidazo[1,2-a]pyridin-6-yl)-4-(methoxy-d₃)-7H-pyrrolo[2,3-d]pyrimidin-2-yl)amino)-1-methylcyclohexan-1-ol [2379];
(1s,4s)-4-((5-(imidazo [1,2-a]pyridin-6-yl)-4-(methoxy-d₃)-7H-pyrrolo [2,3-d] pyrimidin-2-yl)amino)-1-methylcyclohexan-1-ol [2380];
(1s,4s)-1-ethyl-4-45-(imidazo[1,2-a]pyridin-6-yl)-4-methoxy-7H-pyrrolo [2,3-d]pyrimidin-2-yl)amino)cyclohexan-1-01 [2381];
1-(4-((5-(imidazo [1,2-a]pyridin-6-yl)-4-methoxy-7H-pyrrolo [2,3-d]pyrimidin-2-yl)amino)piperidin-1-yl)ethan-1-one [2382];
(S)-5-((5-(imidazo [1,2-a] pyridin-6-yl)-4-methoxy-7H-pyrrolo [2,3-d]pyrimidin-2-yl)amino)-1-methylpiperidin-2-one [2383];
(R)-5-((5-(imidazo [1,2-a]pyridin-6-yl)-4-methoxy-7H-pyrrolo [2,3-d]pyrimidin-2-yl)amino)-1-methylpiperidin-2-one [2384];
1-(2-((5-(imidazo [1,2-a]pyridin-6-yl)-4-methoxy-7H-pyrrolo [2,3-d]pyrimidin-2-yl)amino)-7-azaspiro [3 .5]nonan-7-yl)ethan-1-one [2385];
1-((3aR,5s,6aS)-5-((5-(imidazo [1,2-a]pyridin-6-yl)-4-methoxy-7H-pyrrolo [2,3-d]pyrimidin-2-yl)amino)hexahydrocyclopenta[c]pyrrol-2(1H)-yl)ethan-1-one [2386];
1-((3aR,5r,6aS)-5-((5-(imidazo [1,2-a]pyridin-6-yl)-4-methoxy-7H-pyrrolo [2,3-d] pyrimidin-2-yl)amino)hexahydrocyclopenta[c] pyrrol-2(1H)-yl)ethan-1-one [2387];
1-(7-((5-(imidazo [1,2-a]pyridin-6-yl)-4-methoxy-7H-pyrrolo [2,3-d]pyrimidin-2-yl)amino)-2-azaspiro [3 .5]nonan-2-yl)ethan-1-one [2388];
5-(imidazo [1,2-a] pyridin-6-yl)-4-methoxy-N-(2-oxaspiro [3.5]nonan-7-yl)-7H-pyrrolo [2,3-d]pyrimidin-2-amine [2389];
5-(imidazo [1,2-a] pyridin-6-yl)-4-methoxy-N-(1,4-dioxaspiro [4.5] decan-8-yl)-7H-pyrrolo [2,3-d]pyrimidin-2-amine [2390];
(1s,3s)-3-45-(3-fluoroimidazo [1,2-a]pyridin-6-yl)-7H-pyrrolo [2,3-d] pyrimidin-2-yl)amino)-N,N,1-trimethylcyclobutane-1-carboxamide [2391];
(1r,3r)-3-((5-(3-fluoroimidazo [1,2-a]pyridin-6-yl)-7H-pyrrolo [2,3-d] pyrimidin-2-yl)amino)-N,N,1-trimethylcyclobutane-1-carboxamide [2392];
((1r,30-3-45-(3-fluoroimidazo [1,2-a]pyridin-6-yl)-7H-pyrrolo [2,3-d] pyrimidin-2-yl)amino)-1-methylcyclobutyl)(pyrrolidin-1-yl)methanone [2393];
((1s,3s)-3-((5-(3-fluoroimidazo [1,2-a] pyridin-6-yl)-7H-pyrrolo [2,3-d] pyrimidin-2-yl)amino)-1-methylcyclobutyl)(pyrrolidin-1-yl)methanone [2394];
N-((1s,3s)-3-((5-(3-fluoroimidazo [1,2-a] pyridin-6-yl)-7H-pyrrolo [2,3-d]pyrimidin-2-yl)amino)-1-methylcyclobutypacetamide [2395];
N-((1r,3r)-3-((5-(3-fluoroimidazo [1,2-a] pyridin-6-yl)-7H-pyrrolo [2,3-d]pyrimidin-2-yl)amino)-1-methylcyclobutypacetamide [2396];
N-((1s,3s)-3-((5-(3-fluoroimidazo[1,2-a]pyridin-6-yl)-7H-pyrrolo[2,3-d]pyrimidin-2-yl)amino)-1-methylcyclobutyl)propionamide [2397];
N-((1r,3r)-3-((5-(3-fluoroimidazo[1,2-a]pyridin-6-yl)-7H-pyrrolo[2,3-d]pyrimidin-2-yl)amino)-1-methylcyclobutyl)propionamide [2398];
1-((1r,3r)-3-((5-(3-fluoroimidazo[1,2-a]pyridin-6-yl)-7H-pyrrolo[2,3-d]pyrimidin-2-yl)amino)-1-methylcyclobutyl)pyrrolidin-2-one [2399];
1-((1s,3s)-3-((5-(3-fluoroimidazo[1,2-a]pyridin-6-yl)-7H-pyrrolo[2,3-d]pyrimidin-2-yl)amino)-1-methylcyclobutyl)pyrrolidin-2-one [2400];
(1s,3s)-3-45-(3-fluoroimidazo[1,2-a]pyridin-6-yl)-7H-pyrrolo[2,3-d]pyrimidin-2-yl)amino)-1-methylcyclobutan-1-ol [2401];
(1r,3r)-3-((5-(3-fluoroimidazo[1,2-a]pyridin-6-yl)-7H-pyrrolo[2,3-d]pyrimidin-2-yl)amino)-1-methylcyclobutan-1-ol [2402];
((1s,4s)-4-((5-(3-fluoroimidazo[1,2-a]pyridin-6-yl)-7H-pyrrolo[2,3-d]pyrimidin-2-yl)amino)cyclohexyl)(pyrrolidin-1-yl)methanone [2403];
(1s,4s)-4-45-(3-fluoroimidazo[1,2-a]pyridin-6-yl)-7H-pyrrolo[2,3-d]pyrimidin-2-yl)amino)-1-methylcyclohexan-1-ol [2404];
(1r,40-4-45-(3-fluoroimidazo[1,2-a]pyridin-6-yl)-7H-pyrrolo[2,3-d]pyrimidin-2-yl)amino)-1-methylcyclohexan-1-ol [2405];
1-(4-((5-(3-fluoroimidazo[1,2-a]pyridin-6-yl)-7H-pyrrolo[2,3-d]pyrimidin-2-yl)amino)piperidin-1-yl)ethan-1-one [2406];
(S)-5-((5-(3-fluoroimidazo[1,2-a]pyridin-6-yl)-7H-pyrrolo[2,3-d]pyrimidin-2-yl)amino)-1-methylpiperidin-2-one [2407];
(R)-5-((5-(3-fluoroimidazo[1,2-a]pyridin-6-yl)-7H-pyrrolo[2,3-d]pyrimidin-2-yl)amino)-1-methylpiperidin-2-one [2408];
5-(3-fluoroimidazo[1,2-a]pyridin-6-yl)-N-(1,4-dioxaspiro[4.5]decan-8-yl)-7H-pyrrolo[2,3-d]pyrimidin-2-amine [2409];
(1s,3s)-3-((5-(3-fluoroimidazo[1,2-a]pyridin-6-yl)-4-methoxy-7H-pyrrolo[2,3-d]pyrimidin-2-yl)amino)-N,N,1-trimethylcyclobutane-1-carboxamide [2410];
(1r,3r)-3-((5-(3-fluoroimidazo[1,2-a]pyridin-6-yl)-4-methoxy-7H-pyrrolo[2,3-d]pyrimidin-2-yl)amino)-N,N,1-trimethylcyclobutane-1-carboxamide [2411];
((1s,3s)-3-((5-(3-fluoroimidazo[1,2-a]pyridin-6-yl)-4-methoxy-7H-pyrrolo[2,3-d]pyrimidin-2-yl)amino)-1-methylcyclobutyl)(pyrrolidin-1-yl)methanone [2412];
((1r,3r)-3-((5-(3-fluoroimidazo[1,2-a]pyridin-6-yl)-4-methoxy-7H-pyrrolo[2,3-d]pyrimidin-2-yl)amino)-1-methylcyclobutyl)(pyrrolidin-1-yl)methanone [2413];
N-((1s,3s)-3-((5-(3-fluoroimidazo [1,2-a] pyridin-6-yl)-4-methoxy-7H-pyrrolo [2,3-d] pyrimidin-2-yl)amino)-1-methylcyclobutypacetamide [2414];
N-((1r,3r)-3-((5-(3-fluoroimidazo [1,2-a] pyridin-6-yl)-4-methoxy-7H-pyrrolo [2,3-d] pyrimidin-2-yl)amino)-1-methylcyclobutypacetamide [2415];
N-((1s,3s)-3-((5-(3-fluoroimidazo [1,2-a] pyridin-6-yl)-4-methoxy-7H-pyrrolo [2,3-d] pyrimidin-2-yl)amino)-1-methylcyclobutyl)propionamide [2416];
N-((1r,3r)-3-((5-(3-fluoroimidazo [1,2-a] pyridin-6-yl)-4-methoxy-7H-pyrrolo [2,3-d] pyrimidin-2-yl)amino)-1-methylcyclobutyl)propionamide [2417];
1-((1r,3r)-3-((5-(3-fluoroimidazo [1,2-a] pyridin-6-yl)-4-methoxy-7H-pyrrolo [2,3-d] pyrimidin-2-yl)amino)-1-methylcyclobutyl)pyrrolidin-2-one [2418];
1-((1s,3s)-3-((5-(3-fluoroimidazo [1,2-a] pyridin-6-yl)-4-methoxy-7H-pyrrolo [2,3-d] pyrimidin-2-yl)amino)-1-methylcyclobutyl)pyrrolidin-2-one [2419];
(1s,3s)-3-((5-(3-fluoroimidazo [1,2-a]pyridin-6-yl)-4-methoxy-7H-pyrrolo [2,3-d] pyrimidin-2-yl)amino)-1-methylcyclobutan-1-ol [2420];
(1r,3r)-3-((5-(3-fluoroimidazo [1,2-a]pyridin-6-yl)-4-methoxy-7H-pyrrolo [2,3-d]pyrimidin-2-yl)amino)-1-methylcyclobutan-1-ol [2421];
(1s,4s)-4-((5-(3-fluoroimidazo [1,2-a]pyridin-6-yl)-4-methoxy-7H-pyrrolo [2,3-d] pyrimidin-2-yl)amino)-N,N-dimethylcyclohexane-1-carboxamide [2422];
((l s,4s)-4-((5-(3-fluoroimidazo [1,2-a] pyridin-6-yl)-4-methoxy-7H-pyrrolo [2,3-d]pyrimidin-2-yl)amino)cyclohexyl)(pyrrolidin-1-yl)methanone [2423];
N-((1r,40-4-45-(3-fluoroimidazo [1,2-a] pyridin-6-yl)-4-methoxy-7H-pyrrolo [2,3-d] pyrimidin-2-yl)amino)cyclohexyl)acetamide [2424];
N-((1s,4s)-4-((5-(3-fluoroimidazo [1,2-a] pyridin-6-yl)-4-methoxy-7H-pyrrolo [2,3-d] pyrimidin-2-yl)amino)cyclohexyl)acetamide [2425];
1-((1s,4s)-4-((5-(3-fluoroimidazo [1,2-a] pyridin-6-yl)-4-methoxy-7H-pyrrolo [2,3-d] pyrimidin-2-yl)amino)cyclohexyl)pyrrolidin-2-one [2426];
5-(3-fluoroimidazo [1,2-a]pyridin-6-yl)-4-methoxy-N-((1s,4s)-4-methoxycyclohexyl)-7H-pyrrolo [2,3-d]pyrimidin-2-amine [2427];
2-(((1r,40-4-45-(3-fluoroimidazo [1,2-a] pyridin-6-yl)-4-methoxy-7H-pyrrolo [2,3-d] pyrimidin-2-yl)amino)cyclohexyl)oxy)ethan-1-ol [2428];
2-(((1s,4s)-4-((5-(3-fluoroimidazo [1,2-a]pyridin-6-yl)-4-methoxy-7H-pyrrolo [2,3-d]pyrimidin-2-yl)amino)cyclohexyl)oxy)ethan-1-ol [2429];
N-((1r,40-4-45-(3-fluoroimidazo [1,2-a] pyridin-6-yl)-4-methoxy-7H-pyrrolo [2,3-d] pyrimidin-2-yl)amino)-1-methylcyclohexyl)acetamide [2430];
(1s,4s)-4-((5-(3-fluoroimidazo[1,2-a]pyridin-6-yl)-4-methoxy-7H-pyrrolo[2,3-d]pyrimidin-2-yl)amino)-1-methylcyclohexan-1-ol [2431];
(1r,40-4-45-(3-fluoroimidazo[1,2-a]pyridin-6-yl)-4-methoxy-7H-pyrrolo[2,3-d]pyrimidin-2-yl)amino)-1-methylcyclohexan-1-ol [2432];
(1s,4s)-4-((5-(3-fluoroimidazo[1,2-a]pyridin-6-yl)-4-(methoxy-d₃)-7H-pyrrolo[2,3-d]pyrimidin-2-yl)amino)-1-methylcyclohexan-1-ol [2433];
(1r,40-4-45-(3-fluoroimidazo[1,2-a]pyridin-6-yl)-4-(methoxy-d₃)-7H-pyrrolo[2,3-d]pyrimidin-2-yl)amino)-1-methylcyclohexan-1-ol [2434];
(1s,4s)-1-ethyl-4-((5-(3-fluoroimidazo[1,2-a]pyridin-6-yl)-4-methoxy-7H-pyrrolo[2,3-d]pyrimidin-2-yl)amino)cyclohexan-1-ol [2435];
1-(4-((5-(3-fluoroimidazo[1,2-a]pyridin-6-yl)-4-methoxy-7H-pyrrolo[2,3-d]pyrimidin-2-yl)amino)piperidin-1-yl)ethan-1-one [2436];
(R)-5-((5-(3-fluoroimidazo[1,2-a]pyridin-6-yl)-4-methoxy-7H-pyrrolo[2,3-d]pyrimidin-2-yl)amino)-1-methylpiperidin-2-one [2437];
(S)-5-((5-(3-fluoroimidazo[1,2-a]pyridin-6-yl)-4-methoxy-7H-pyrrolo[2,3-d]pyrimidin-2-yl)amino)-1-methylpiperidin-2-one [2438];
1-(2-((5-(3-fluoroimidazo[1,2-a]pyridin-6-yl)-4-methoxy-7H-pyrrolo[2,3-d]pyrimidin-2-yl)amino)-7-azaspiro[3.5]nonan-7-yl)ethan-1-one [2439];
1-((3aR,5s,6aS)-5-((5-(3-fluoroimidazo[1,2-a]pyridin-6-yl)-4-methoxy-7H-pyrrolo[2,3-d]pyrimidin-2-yl)amino)hexahydrocyclopenta[c]pyrrol-2(1H)-yl)ethan-1-one [2440];
1-((3aR,5r,6aS)-5-((5-(3-fluoroimidazo[1,2-a]pyridin-6-yl)-4-methoxy-7H-pyrrolo[2,3-d]pyrimidin-2-yl)amino)hexahydrocyclopenta[c]pyrrol-2(1H)-yl)ethan-1-one [2441];
1-(7-((5-(3-fluoroimidazo[1,2-a]pyridin-6-yl)-4-methoxy-7H-pyrrolo[2,3-d]pyrimidin-2-yl)amino)-2-azaspiro[3.5]nonan-2-yl)ethan-1-one [2442];
5-(3-fluoroimidazo[1,2-a]pyridin-6-yl)-4-methoxy-N-(2-oxaspiro[3.5]nonan-7-yl)-7H-pyrrolo[2,3-d]pyrimidin-2-amine [2443];
5-(3-fluoroimidazo[1,2-a]pyridin-6-yl)-4-methoxy-N-(1,4-dioxaspiro[4.5]decan-8-yl)-7H-pyrrolo[2,3-d]pyrimidin-2-amine [2444];
(1s,3s)-3-45-(8-fluoroimidazo[1,2-a]pyridin-6-yl)-7H-pyrrolo[2,3-d]pyrimidin-2-yl)amino)-N,N,1-trimethylcyclobutane-1-carboxamide [2445];
(1r,3r)-3-((5-(8-fluoroimidazo[1,2-a]pyridin-6-yl)-7H-pyrrolo[2,3-d]pyrimidin-2-yl)amino)-N,N,1-trimethylcyclobutane-1-carboxamide [2446];
((1s,3s)-3-((5-(8-fluoroimidazo[1,2-a]pyridin-6-yl)-7H-pyrrolo[2,3-d]pyrimidin-2-yl)amino)-1-methylcyclobutyl)(pyrrolidin-1-yl)methanone [2447];
((1r, 3r)-3((5-(5-(8-fluoroimidazo [1,2-a]pyridin-6-yl)-7H-pyrrolo [2,3-d] pyrimidin-2-yl)amino)-1-methylcyclobutyl)(pyrrolidin-1-yl)methanone [2448];
N-((1s,3s)-3-((5-(8-fluoroimidazo [1,2-a] pyridin-6-yl)-7H-pyrrolo [2,3-d]pyrimidin-2-yl)amino)-1-methylcyclobutypacetamide [2449];
N-((1r,3r)-3-((5-(8-fluoroimidazo [1,2-a] pyridin-6-yl)-7H-pyrrolo [2,3-d]pyrimidin-2-yl)amino)-1-methylcyclobutypacetamide [2450];
N-((1s,3s)-3-((5-(8-fluoroimidazo [1,2-a] pyridin-6-yl)-7H-pyrrolo [2,3-d]pyrimidin-2-yl)amino)-1-methylcyclobutyl)propionamide [2451];
N-((1r,3r)-3-((5-(8-fluoroimidazo [1,2-a] pyridin-6-yl)-7H-pyrrolo [2,3-d]pyrimidin-2-yl)amino)-1-methylcyclobutyl)propionamide [2452];
1-((1r,3r)-3-((5-(8-fluoroimidazo [1,2-a] pyridin-6-yl)-7H-pyrrolo [2,3-d] pyrimidin-2-yl)amino)-1-methylcyclobutyl)pyrrolidin-2-one [2453];
1-((1s,3s)-3-((5-(8-fluoroimidazo [1,2-a] pyridin-6-yl)-7H-pyrrolo [2,3-d]pyrimidin-2-yl)amino)-1-methylcyclobutyl)pyrrolidin-2-one [2454];
(1r,3r)-3-((5-(8-fluoroimidazo [1,2-a]pyridin-6-yl)-7H-pyrrolo [2,3-d] pyrimidin-2-yl)amino)-1-methylcyclobutan-1-ol [2455];
(1s,3s)-3-45-(8-fluoroimidazo [1,2-a]pyridin-6-yl)-7H-pyrrolo [2,3-d] pyrimidin-2-yl)amino)-1-methylcyclobutan-1-ol [2456];
(1s,4s)-4-45-(8-fluoroimidazo [1,2-a]pyridin-6-yl)-7H-pyrrolo [2,3-d] pyrimidin-2-yl)amino)-N,N-dimethylcyclohexane-1-carboxamide [2457];
((1s,4s)-4-((5-(8-fluoroimidazo [1,2-a] pyridin-6-yl)-7H-pyrrolo [2,3-d] pyrimidin-2-yl)amino)cyclohexyl)(pyrrolidin-1-yl)methanone [2458];
N-((1r,4r)-4-((5-(8-fluoroimidazo [1,2-a] pyridin-6-yl)-7H-pyrrolo [2,3-d]pyrimidin-2-yl)amino)cyclohexyl)acetamide [2459];
N-((1s,4s)-4-((5-(8-fluoroimidazo [1,2-a] pyridin-6-yl)-7H-pyrrolo [2,3-d]pyrimidin-2-yl)amino)cyclohexyl)acetamide [2460];
1-((1s,4s)-4-((5-(8-fluoroimidazo [1,2-a] pyridin-6-yl)-7H-pyrrolo [2,3-d]pyrimidin-2-yl)amino)cyclohexyl)pyrrolidin-2-one [2461];
2-(((1r,4r)-4-((5-(8-fluoroimidazo [1,2-a] pyridin-6-yl)-7H-pyrrolo [2,3-d]pyrimidin-2-yl)amino)cyclohexyl)oxy)ethan-1-ol [2462];
N-((1r,40-4-45-(8-fluoroimidazo [1,2-a] pyridin-6-yl)-7H-pyrrolo [2,3-d]pyrimidin-2-yl)amino)-1-methylcyclohexyl)acetamide [2463];
(1s,4s)-1-ethyl-4-((5-(8-fluoroimidazo [1,2-a]pyridin-6-yl)-7H-pyrrolo [2,3-d]pyrimidin-2-yl)amino)cyclohexan-1-01 [2464];
1-(4-((5-(8-fluoroimidazo [1,2-a]pyridin-6-yl)-7H-pyrrolo [2,3-d]pyrimidin-2-yl)amino)piperidin-1-yl)ethan-1-one [2465];
(R)-5-((5-(8-fluoroimidazo [1,2-a]pyridin-6-yl)-7H-pyrrolo [2,3-d]pyrimidin-2-yl)amino)-1-methylpiperidin-2-one [2466];
(S)-5-((5-(8-fluoroimidazo [1,2-a]pyridin-6-yl)-7H-pyrrolo [2,3-d]pyrimidin-2-yl)amino)-1-methylpiperidin-2-one [2467];
1-(2-((5-(8-fluoroimidazo [1,2-a]pyridin-6-yl)-7H-pyrrolo [2,3-d]pyrimidin-2-yl)amino)-7-azaspiro [3 .5]nonan-7-yl)ethan-1-one [2468];
1-((3aR,5s,6aS)-5-((5-(8-fluoroimidazo [1,2-a]pyridin-6-yl)-7H-pyrrolo [2,3-d]pyrimidin-2-yl)amino)hexahydrocyclopenta[c]pyrrol-2(1H)-yl)ethan-1-one [2469];
1-((3aR,5r,6aS)-5-((5-(8-fluoroimidazo [1,2-a] pyridin-6-yl)-7H-pyrrolo [2,3-d]pyrimidin-2-yl)amino)hexahydrocyclopenta[c]pyrrol-2(1H)-yl)ethan-1-one [2470];
1-(7-((5-(8-fluoroimidazo [1,2-a]pyridin-6-yl)-7H-pyrrolo [2,3-d]pyrimidin-2-yl)amino)-2-azaspiro [3 .5]nonan-2-yl)ethan-1-one [2471];
5-(8-fluoroimidazo [1,2-a]pyridin-6-yl)-N-(2-oxaspiro [3.5]nonan-7-yl)-7H-pyrrolo [2,3-d]pyrimidin-2-amine [2472];
5-(8-fluoroimidazo [1,2-a]pyridin-6-yl)-N-(1,4-dioxaspiro [4.5] decan-8-yl)-7H-pyrrolo [2,3-d]pyrimidin-2-amine [2473];
(1s,3s)-3-((5-(8-fluoroimidazo [1,2-a]pyridin-6-yl)-4-methoxy-7H-pyrrolo [2,3-d] pyrimidin-2-yl)amino)-N,N,1-trimethylcyclobutane-1-carboxamide [2474];
(1r,3r)-3-((5-(8-fluoroimidazo [1,2-a]pyridin-6-yl)-4-methoxy-7H-pyrrolo [2,3-d]pyrimidin-2-yl)amino)-N,N,1-trimethylcyclobutane-1-carboxamide [2475];
((l s,3s)-3-((5-(8-fluoroimidazo [1,2-a] pyridin-6-yl)-4-methoxy-7H-pyrrolo [2,3-d]pyrimidin-2-yl)amino)-1-methylcyclobutyl)(pyrrolidin-1-yl)methanone [2476];
((1r,3r)-3-((5-(8-fluoroimidazo [1,2-a]pyridin-6-yl)-4-methoxy-7H-pyrrolo [2,3-d] pyrimidin-2-yl)amino)-1-methylcyclobutyl)(pyrrolidin-1-yl)methanone [2477];
N-((1s,3s)-3-((5-(8-fluoroimidazo [1,2-a] pyridin-6-yl)-4-methoxy-7H-pyrrolo [2,3-d] pyrimidin-2-yl)amino)-1-methylcyclobutypacetamide [2478];
N-((1r,3r)-3-((5-(8-fluoroimidazo [1,2-a] pyridin-6-yl)-4-methoxy-7H-pyrrolo [2,3-d] pyrimidin-2-yl)amino)-1-methylcyclobutypacetamide [2479];
N-((1s,3s)-3-((5-(8-fluoroimidazo [1,2-a] pyridin-6-yl)-4-methoxy-7H-pyrrolo [2,3-d] pyrimidin-2-yl)amino)-1-methylcyclobutyl)propionamide [2480];
N-((1r,3r)-3-((5-(8-fluoroimidazo [1,2-a] pyridin-6-yl)-4-methoxy-7H-pyrrolo [2,3-d] pyrimidin-2-yl)amino)-1-methylcyclobutyl)propionamide [2481];
1-((1 s,3 s)-3-((5-(8-fluoroimidazo [1,2-a] pyridin-6-yl)-4-methoxy-7H-pyrrolo [2,3-d] pyrimidin-2-yl)amino)-1-methylcyclobutyppyrrolidin-2-one [2482];
1-((1r,3r)-3-((5-(8-fluoroimidazo [1,2-a] pyridin-6-yl)-4-methoxy-7H-pyrrolo [2,3-d] pyrimidin-2-yl)amino)-1-methylcyclobutyppyrrolidin-2-one [2483];
(1 s,3 s)-3-((5-(8-fluoroimidazo [1,2-a]pyridin-6-yl)-4-methoxy-7H-pyrrolo [2,3-d] pyrimidin-2-yl)amino)-1-methylcyclobutan-1-ol [2484];
(1r,3r)-3-((5-(8-fluoroimidazo [1,2-a]pyridin-6-yl)-4-methoxy-7H-pyrrolo [2,3-d]pyrimidin-2-yl)amino)-1-methylcyclobutan-1-ol [2485];
(1 s,4 s)-4-((5-(8-fluoroimidazo [1,2-a]pyridin-6-yl)-4-methoxy-7H-pyrrolo [2,3-d] pyrimidin-2-yl)amino)-N,N-dimethylcyclohexane-1-carboxamide [2486];
((1 s,4 s)-4-((5-(8-fluoroimidazo [1,2-a] pyridin-6-yl)-4-methoxy-7H-pyrrolo [2,3-d]pyrimidin-2-yl)amino)cyclohexyl)(pyrrolidin-1-yl)methanone [2487];
N-((1r,40-4-45-(8-fluoroimidazo [1,2-a] pyridin-6-yl)-4-methoxy-7H-pyrrolo [2,3-d]pyrimidin-2-yl)amino)cyclohexypacetamide [2488];
N-((1 s,4 s)-4-((5-(8-fluoroimidazo [1,2-a] pyridin-6-yl)-4-methoxy-7H-pyrrolo [2,3-d]pyrimidin-2-yl)amino)cyclohexypacetamide [2489];
1-((1 s,4 s)-4-((5-(8-fluoroimidazo [1,2-a] pyridin-6-yl)-4-methoxy-7H-pyrrolo [2,3-d]pyrimidin-2-yl)amino)cyclohexyl)pyrrolidin-2-one [2490];
2-(((1r,40-4-45-(8-fluoroimidazo [1,2-a] pyridin-6-yl)-4-methoxy-7H-pyrrolo [2,3-d]pyrimidin-2-yl)amino)cyclohexyl)oxy)ethan-1-ol [2491];
2-(((1 s,4 s)-4-((5-(8-fluoroimidazo[1,2-a]pyridin-6-yl)-4-methoxy-7H-pyrrolo [2,3-d]pyrimidin-2-yl)amino)cyclohexyl)oxy)ethan-1-ol [2492];
N-((1r,40-4-45-(8-fluoroimidazo [1,2-a] pyridin-6-yl)-4-methoxy-7H-pyrrolo [2,3-d] pyrimidin-2-yl)amino)-1-methylcyclohexyl)acetamide [2493];
(1r,4r) -44(5-(8-fluoroimidazo [1,2-a]pyridin-6-yl)-4-methoxy-7H-pyrrolo [2,3-d]pyrimidin-2-yl)amino)-1-methylcyclohexan-1-ol [2494];
(1 s,4 s)-4-((5-(8-fluoroimidazo [1,2-a]pyridin-6-yl)-4-(methoxy-d₃)-7H-pyrrolo [2,3-d] pyrimidin-2-yl)amino)-1-methylcyclohexan-1-ol [2495];
(1r,4r) -44(5-(8-fluoroimidazo [1,2-a]pyridin-6-yl)-4-(methoxy-d₃)-7H-pyrrolo [2,3-d]pyrimidin-2-yl)amino)-1-methylcyclohexan-1-ol [2496];
(1 s,4 s)-1-ethyl-4-((5-(8-fluoroimidazo [1,2-a]pyridin-6-yl)-4-methoxy-7H-pyrrolo [2,3-d]pyrimidin-2-yl)amino)cyclohexan-1-ol [2497];
1-(4-((5-(8-fluoroimidazo [1,2-a]pyridin-6-yl)-4-methoxy-7H-pyrrolo [2,3-d]pyrimidin-2-yl)amino)piperidin-1-yl)ethan-1-one [2498];
(S)-5-((5-(8-fluoroimidazo[1,2-a]pyridin-6-yl)-4-methoxy-7H-pyrrolo[2,3-d]pyrimidin-2-yl)amino)-1-methylpiperidin-2-one [2499];
(R)-5-((5-(8-fluoroimidazo[1,2-a]pyridin-6-yl)-4-methoxy-7H-pyrrolo[2,3-d]pyrimidin-2-yl)amino)-1-methylpiperidin-2-one [2500]; or a pharmaceutically acceptable salt thereof.

28. The compound of claim 1, wherein the compound of Formula I is selected from the group consisting of:
1-(2-((5-(8-fluoroimidazo[1,2-a]pyridin-6-yl)-4-methoxy-7H-pyrrolo[2,3-d]pyrimidin-2-yl)amino)-7-azaspiro [3 .5] nonan-7-yl)ethan-1-one [2501];
1-((3aR,5s,6aS)-5-((5-(8-fluoroimidazo [1,2-a]pyridin-6-yl)-4-methoxy-7H-pyrrolo [2,3-d]pyrimidin-2-yl)amino)hexahydrocyclopenta[c]pyrrol-2(1H)-yl)ethan-1-one [2502];
1-((3aR,5r,6aS)-5-((5-(8-fluoroimidazo [1,2-a]pyridin-6-yl)-4-methoxy-7H-pyrrolo [2,3-d]pyrimidin-2-yl)amino)hexahydrocyclopenta[c]pyrrol-2(1H)-yl)ethan-1-one [2503];
1-(7-((5-(8-fluoroimidazo[1,2-a]pyridin-6-yl)-4-methoxy-7H-pyrrolo[2,3-d]pyrimidin-2-yl)amino)-2-azaspiro [3 .5] nonan-2-yl)ethan-1-one [2504];
5-(8-fluoroimidazo[1,2-a]pyridin-6-yl)-4-methoxy-N-(2-oxaspiro[3.5]nonan-7-yl)-7H-pyrrolo[2,3-d]pyrimidin-2-amine [2505];
(1s,3s)-3-((5-(imidazo [1,2-a]pyrimidin-6-yl)-7H-pyrrolo [2,3-d] pyrimidin-2-yl)amino)-N,N,1-trimethylcyclobutane-1-carboxamide [2506];
(1r,3r)-3-((5-(imidazo[1,2-a]pyrimidin-6-yl)-7H-pyrrolo[2,3-d]pyrimidin-2-yl)amino)-N,N,1-trimethylcyclobutane-1-carboxamide [2507];
((1s,3s)-3-((5-(imidazo [1,2-a] pyrimidin-6-yl)-7H-pyrrolo [2,3-d] pyrimidin-2-yl)amino)-1-methylcyclobutyl)(pyrrolidin-1-yl)methanone [2508];
((1r,3 r) -3-((5-(imidazo [1,2-a] pyrimidin-6-yl)-7H-pyrrolo [2,3-d] pyrimidin-2-yl)amino) -1-methylcyclobutyl)(pyrrolidin-1-yl)methanone [2509];
N-((1s,3s)-3-((5-(imidazo [1,2-a] pyrimidin-6-yl)-7H-pyrrolo [2,3-d] pyrimidin-2-yl)amino)-1-methylcyclobutypacetamide [2510];
N-((1r,3r)-3-((5-(imidazo [1,2-a] pyrimidin-6-yl)-7H-pyrrolo [2,3-d] pyrimidin-2-yl)amino)-1-methylcyclobutypacetamide [2511];
N-((1r,3r)-3-((5-(imidazo [1,2-a] pyrimidin-6-yl)-7H-pyrrolo [2,3-d] pyrimidin-2-yl)amino)-1-methylcyclobutyl)propionamide [2512];
N-((1s,3s)-3-((5-(imidazo [1,2-a] pyrimidin-6-yl)-7H-pyrrolo [2,3-d] pyrimidin-2-yl)amino)-1-methylcyclobutyl)propionamide [2513];
1-((1r,3r)-3-((5-(imidazo[1,2-a]pyrimidin-6-yl)-7H-pyrrolo[2,3-d]pyrimidin-2-yl)amino)-1-methylcyclobutyl)pyrrolidin-2-one [2514];
1-((1s,3s)-3-((5-(imidazo [1,2-a] pyrimidin-6-yl)-7H-pyrrolo [2,3-d]pyrimidin-2-yl)amino)-1-methylcyclobutyl)pyrrolidin-2-one [2515];
(1r,3r)-3-((5-(imidazo [1,2-a]pyrimidin-6-yl)-7H-pyrrolo [2,3-d]pyrimidin-2-yl)amino)-1-methylcyclobutan-1-ol [2516];
(1s,3s)-3-((5-(imidazo [1,2-a]pyrimidin-6-yl)-7H-pyrrolo [2,3-d]pyrimidin-2-yl)amino)-1-methylcyclobutan-1-ol [2517];
(1s,4s)-4-((5-(imidazo [1,2-a]pyrimidin-6-yl)-7H-pyrrolo [2,3-d]pyrimidin-2-yl)amino)-N,N-dimethylcyclohexane-1-carboxamide [2518];
((1s,4s)-4-((5-(imidazo [1,2-a]pyrimidin-6-yl)-7H-pyrrolo [2,3-d]pyrimidin-2-yl)amino)cyclohexyl)(pyrrolidin-1-yl)methanone [2519];
N-((1s,4s)-4-((5-(imidazo [1,2-a] pyrimidin-6-yl)-7H-pyrrolo [2,3-d]pyrimidin-2-yl)amino)cyclohexypacetamide [2520];
N-((1r,4r)-4-((5-(imidazo [1,2-a] pyrimidin-6-yl)-7H-pyrrolo [2,3-d]pyrimidin-2-yl)amino)cyclohexypacetamide [2521];
1-((1s,4s)-4-((5-(imidazo [1,2-a] pyrimidin-6-yl)-7H-pyrrolo [2,3-d]pyrimidin-2-yl)amino)cyclohexyl)pyrrolidin-2-one [2522];
N-((1r,40-4-45-(imidazo [1,2-a] pyrimidin-6-yl)-7H-pyrrolo [2,3-d]pyrimidin-2-yl)amino)-1-methylcyclohexyl)acetamide [2523];
(1s,4s)-4-((5-(imidazo [1,2-a]pyrimidin-6-yl)-7H-pyrrolo [2,3-d]pyrimidin-2-yl)amino)-1-methylcyclohexan-1-ol [2524];
(1r,40-4-45-(imidazo [1,2-a]pyrimidin-6-yl)-7H-pyrrolo [2,3-d]pyrimidin-2-yl)amino)-1-methylcyclohexan-1-ol [2525];
(1s,4s)-1-ethyl-4-((5-(imidazo[1,2-a]pyrimidin-6-yl)-7H-pyrrolo [2,3-d] pyrimidin-2-yl)amino)cyclohexan-1-01 [2526];
1-(4-((5-(imidazo [1,2-a]pyrimidin-6-yl)-7H-pyrrolo [2,3-d]pyrimidin-2-yl)amino)piperidin-1-yl)ethan-1-one [2527];
(S)-5-((5-(imidazo [1,2-a] pyrimidin-6-yl)-7H-pyrrolo [2,3-d]pyrimidin-2-yl)amino)-1-methylpiperidin-2-one [2528];
(R)-5-((5-(imidazo [1,2-a]pyrimidin-6-yl)-7H-pyrrolo [2,3-d] pyrimidin-2-yl)amino)-1-methylpiperidin-2-one [2529];
1-(2-((5-(imidazo [1,2-a]pyrimidin-6-yl)-7H-pyrrolo [2,3-d]pyrimidin-2-yl)amino)-7-azaspiro [3 .5]nonan-7-yl)ethan-1-one [2530];
1-((3aR,5s,6aS)-5-((5-(imidazo [1,2-a]pyrimidin-6-yl)-7H-pyrrolo [2,3-d]pyrimidin-2-yl)amino)hexahydrocyclopenta[c]pyrrol-2(1H)-yl)ethan-1-one [2531];
1-((3 aR,5 r,6aS)-5-((5-(imidazo [1,2-a]pyrimidin-6-yl)-7H-pyrrolo [2,3-d]pyrimidin-2-yl)amino)hexahydrocyclopenta[c]pyrrol-2(1H)-yl)ethan-1-one [2532];
1-(7-((5-(imidazo [1,2-a]pyrimidin-6-yl)-7H-pyrrolo [2,3-d]pyrimidin-2-yl)amino)-2-azaspiro [3 .5]nonan-2-yl)ethan-1-one [2533];
5-(imidazo [1,2-a] pyrimidin-6-yl)-N-(2-oxaspiro [3 .5]nonan-7-yl)-7H-pyrrolo [2,3-d]pyrimidin-2-amine [2534];
5-(imidazo [1,2-a] pyrimidin-6-yl)-N-(1,4-dioxaspiro [4 .5] decan-8-yl)-7H-pyrrolo [2,3-d]pyrimidin-2-amine [2535];
(1 s,3 s)-3-((5-(imidazo[1,2-a]pyrimidin-6-yl)-4-methoxy-7H-pyrrolo [2,3-d]pyrimidin-2-yl)amino)-N,N, 1-trimethylcyclobutane-1-carboxamide [2536];
(1r,3r)-3-((5-(imidazo [1 ,2-a]pyrimidin-6-yl)-4-methoxy-7H-pyrrolo [2,3-d]pyrimidin-2-yl)amino)-N,N, 1-trimethylcyclobutane-1-carboxamide [2537];
((1 s,3 s)-3-((5-(imidazo [1,2-a]pyrimidin-6-yl)-4-methoxy-7H-pyrrolo [2,3-d]pyrimidin-2-yl)amino)-1-methylcyclobutyl)(pyrrolidin-1-yl)methanone [2538];
((1r,3r)-3-((5-(imidazo[1,2-a]pyrimidin-6-yl)-4-methoxy-7H-pyrrolo [2,3-d] pyrimidin-2-yl)amino)-1-methylcyclobutyl)(pyrrolidin-1-yl)methanone [2539];
N-((1 s,3 s)-3-((5-(imidazo [1,2-a] pyrimidin-6-yl)-4-methoxy-7H-pyrrolo [2,3-d]pyrimidin-2-yl)amino)-1-methylcyclobutypacetamide [2540];
N-((1r,3 r)-3-((5-(imidazo [1,2-a] pyrimidin-6-yl)-4-methoxy-7H-pyrrolo [2,3-d]pyrimidin-2-yl)amino)-1-methylcyclobutypacetamide [2541];
N-((1 s,3 s)-3-((5-(imidazo [1,2-a] pyrimidin-6-yl)-4-methoxy-7H-pyrrolo [2,3-d]pyrimidin-2-yl)amino)-1-methylcyclobutyppropionamide [2542];
N-((1r,3r)-3-((5-(imidazo [1,2-a] pyrimidin-6-yl)-4-methoxy-7H-pyrrolo [2,3-d]pyrimidin-2-yl)amino)-1-methylcyclobutyppropionamide [2543];
1-((1r,3r)-3-((5-(imidazo [1,2-a]pyrimidin-6-yl)-4-methoxy-7H-pyrrolo [2,3-d]pyrimidin-2-yl)amino)-1-methylcyclobutyppyrrolidin-2-one [2544];
1-((1 s,3 s)-3-((5-(imidazo [1,2-a] pyrimidin-6-yl)-4-methoxy-7H-pyrrolo [2,3-d]pyrimidin-2-yl)amino)-1-methylcyclobutyppyrrolidin-2-one [2545];
(1r,3r)-3-((5-(imidazo [1 ,2-a]pyrimidin-6-yl)-4-methoxy-7H-pyrrolo [2,3-d] pyrimidin-2-yl)amino)-1-methylcyclobutan-1-ol [2546];
(1 s,3 s)-3-((5-(imidazo[1,2-a]pyrimidin-6-yl)-4-methoxy-7H-pyrrolo [2,3-d] pyrimidin-2-yl)amino)-1-methylcyclobutan-1-ol [2547];
(1 s,4 s)-4-((5-(imidazo[1,2-a]pyrimidin-6-yl)-4-methoxy-7H-pyrrolo [2,3-d] pyrimidin-2-yl)amino)-N,N-dimethylcyclohexane-1-carboxamide [2548];
((1s,4s)-4-((5-(imidazo [1,2-a]pyrimidin-6-yl)-4-methoxy-7H-pyrrolo [2,3-d]pyrimidin-2-yl)amino)cyclohexyl)(pyrrolidin-1-yl)methanone [2549];
N-((1s,4s)-4-((5-(imidazo [1,2-a] pyrimidin-6-yl)-4-methoxy-7H-pyrrolo [2,3-d]pyrimidin-2-yl)amino)cyclohexypacetamide [2550];
N-((1r,40-4-45-(imidazo [1,2-a] pyrimidin-6-yl)-4-methoxy-7H-pyrrolo [2,3-d]pyrimidin-2-yl)amino)cyclohexypacetamide [2551];
1-((1s,4s)-4-((5-(imidazo [1,2-a] pyrimidin-6-yl)-4-methoxy-7H-pyrrolo [2,3-d]pyrimidin-2-yl)amino)cyclohexyl)pyrrolidin-2-one [2552];
5-(imidazo [1,2-a] pyrimidin-6-yl)-4-methoxy-N-((1s,4s)-4-methoxycyclohexyl)-7H-pyrrolo [2,3-d]pyrimidin-2-amine [2553];
N-((1r,40-4-45-(imidazo [1,2-a] pyrimidin-6-yl)-4-methoxy-7H-pyrrolo [2,3-d]pyrimidin-2-yl)amino)-1-methylcyclohexyl)acetamide [2554];
(1r,40-4-45-(imidazo [1,2-a]pyrimidin-6-yl)-4-methoxy-7H-pyrrolo [2,3-d] pyrimidin-2-yl)amino)-1-methylcyclohexan-1-ol [2555];
(1s,4s)-4-((5-(imidazo [1,2-a]pyrimidin-6-yl)-4-methoxy-7H-pyrrolo [2,3-d]pyrimidin-2-yl)amino)-1-methylcyclohexan-1-ol [2556];
(1r,40-4-45-(imidazo [1,2-a]pyrimidin-6-yl)-4-(methoxy-d₃)-7H-pyrrolo [2,3-d]pyrimidin-2-yl)amino)-1-methylcyclohexan-1-ol [2557];
(1s,4s)-4-((5-(imidazo [1,2-a]pyrimidin-6-yl)-4-(methoxy-d₃)-7H-pyrrolo [2,3-d]pyrimidin-2-yl)amino)-1-methylcyclohexan-1-ol [2558];
(1s,4s)-1-ethyl-4-((5-(imidazo[1,2-a]pyrimidin-6-yl)-4-methoxy-7H-pyrrolo [2,3-d] pyrimidin-2-yl)amino)cyclohexan-1-01 [2559];
1-(4-((5-(imidazo [1,2-a]pyrimidin-6-yl)-4-methoxy-7H-pyrrolo [2,3-d]pyrimidin-2-yl)amino)piperidin-1-yl)ethan-1-one [2560];
(S)-5-((5-(imidazo [1,2-a] pyrimidin-6-yl)-4-methoxy-7H-pyrrolo [2,3-d]pyrimidin-2-yl)amino)-1-methylpiperidin-2-one [2561];
(R)-5-((5-(imidazo [1,2-a]pyrimidin-6-yl)-4-methoxy-7H-pyrrolo [2,3-d]pyrimidin-2-yl)amino)-1-methylpiperidin-2-one [2562];
1-(2-((5-(imidazo [1,2-a]pyrimidin-6-yl)-4-methoxy-7H-pyrrolo [2,3-d]pyrimidin-2-yl)amino)-7-azaspiro [3 .5]nonan-7-yl)ethan-1-one [2563];
1-((3aR,5s,6aS)-5-((5-(imidazo [1,2-a]pyrimidin-6-yl)-4-methoxy-7H-pyrrolo [2,3-d]pyrimidin-2-yl)amino)hexahydrocyclopenta[c]pyrrol-2(1H)-yl)ethan-1-one [2564];
1-((3aR,5r,6aS)-5-((5-(imidazo [1,2-a]pyrimidin-6-yl)-4-methoxy-7H-pyrrolo [2,3-d] pyrimidin-2-yl)amino)hexahydrocyclopenta[c]pyrrol-2(1H)-yl)ethan-1-one [2565];
1-(7-((5-(imidazo [1,2-a]pyrimidin-6-yl)-4-methoxy-7H-pyrrolo [2,3-d]pyrimidin-2-yl)amino)-2-azaspiro [3 .5]nonan-2-yl)ethan-1-one [2566];
5-(imidazo [1,2-a]pyrimidin-6-yl)-4-methoxy-N-(2-oxaspiro [3 .5]nonan-7-yl)-7H-pyrrolo [2,3-d]pyrimidin-2-amine [2567];
5-(imidazo [1,2-a]pyrimidin-6-yl)-4-methoxy-N-(1,4-dioxaspiro [4.5] decan-8-yl)-7H-pyrrolo [2,3-d]pyrimidin-2-amine [2568];
(1s,3s)-3-((5-(1-(2,2-difluoroethyl)-2-methyl-1H-benzo [d] imidazol-6-yl)-7H-pyrrolo [2,3-d]pyrimidin-2-yl)amino)-N,N,1-trimethylcyclobutane-1-carboxamide [2569];
(1r,3r)-3-((5-(1-(2,2-difluoroethyl)-2-methyl-1H-benzo [d] imidazol-6-yl)-7H-pyrrolo [2,3-d]pyrimidin-2-yl)amino)-N,N,1-trimethylcyclobutane-1-carboxamide [2570];
((1r,3r)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)(pyrrolidin-1-yl)methanone [2571];
((1s,3s)-3-((5-(1-(2,2-difluoroethyl)-2-methyl-1H-benzo[d]imidazol-6-yl)-7H-pyrrolo [2,3-d]pyrimidin-2-yl)amino)-1-methylcyclobutyl)(pyrrolidin-1-yl)methanone [2572];
N-((1s,3s)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)acetamide [2573];
N-((1r,3r)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)acetamide [2574];
N-((1s,3s)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)propionamide [2575];
N-((1r,3r)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)propionamide [2576];
1-((1r,3r)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)pyrrolidin-2-one [2577];
1-((1s,3s)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutyl)pyrrolidin-2-one [2578];
(1s,3s)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutan-1-ol [2579];
(1s,3s)-3-((5-(1-(2,2-difluoroethyl)-2-methyl-1H-benzo [d] imidazol-6-yl)-7H-pyrrolo [2,3-d]pyrimidin-2-yl)amino)-1-methylcyclobutan-1-ol [2580];
(1s,4s)-4-((5-(1-(2,2-difluoroethyl)-2-methyl-1H-benzo [d] imidazol-6-yl)-7H-pyrrolo [2,3-d]pyrimidin-2-yl)amino)-N,N-dimethylcyclohexane-1-carboxamide [2581];
((1s,4s)-4-((5-(1-(2,2-difluoroethyl)-2-methyl-1H-benzo[d]imidazol-6-yl)-7H-pyrrolo [2,3-d]pyrimidin-2-yl)amino)cyclohexyl)(pyrrolidin-1-yl)methanone [2582];
N-((1s,4s)-4-((5-(1-(2,2-difluoroethyl)-2-methyl-1H-benzo[d]imidazol-6-yl)-7H-pyrrolo[2,3-d]pyrimidin-2-yl)amino)cyclohexypacetamide [2583];
N-((1r,4r)-4-((5-(1-(2,2-difluoroethyl)-2-methyl-1H-benzo[d]imidazol-6-yl)-7H-pyrrolo[2,3-d]pyrimidin-2-yl)amino)cyclohexypacetamide [2584];
1-((1s,4s)-4-((5-(1-(2,2-difluoroethyl)-2-methyl-1H-benzo[d]imidazol-6-yl)-7H-pyrrolo[2,3-d]pyrimidin-2-yl)amino)cyclohexyl)pyrrolidin-2-one [2585];
2-(((1s,4s)-4-((5-(1-(2,2-difluoroethyl)-2-methyl-1H-benzo[d]imidazol-6-yl)-7H-pyrrolo[2,3-d]pyrimidin-2-yl)amino)cyclohexyl)oxy)ethan-1-ol [2586];
2-(((1r,4r)-4-((5-(1-(2,2-difluoroethyl)-2-methyl-1H-benzo[d]imidazol-6-yl)-7H-pyrrolo[2,3-d]pyrimidin-2-yl)amino)cyclohexyl)oxy)ethan-1-ol [2587];
N-((1r,4r)-4-((5-(1-(2,2-difluoroethyl)-2-methyl-1H-benzo[d]imidazol-6-yl)-7H-pyrrolo[2,3-d]pyrimidin-2-yl)amino)-1-methylcyclohexyl)acetamide [2588];
(1r,4r)-4-((5-(1-(2,2-difluoroethyl)-2-methyl-1H-benzo[d]imidazol-6-yl)-7H-pyrrolo[2,3-d]pyrimidin-2-yl)amino)-1-methylcyclohexan-1-ol [2589];
(1s,4s)-4-((5-(1-(2,2-difluoroethyl)-2-methyl-1H-benzo[d]imidazol-6-yl)-7H-pyrrolo[2,3-d]pyrimidin-2-yl)amino)-1-methylcyclohexan-1-ol [2590];
(1s,4s)-4-((5-(1-(2,2-difluoroethyl)-2-methyl-1H-benzo[d]imidazol-6-yl)-7H-pyrrolo[2,3-d]pyrimidin-2-yl)amino)-1-ethylcyclohexan-1-ol [2591];
1-(4-((5-(1-(2,2-difluoroethyl)-2-methyl-1H-benzo[d]imidazol-6-yl)-7H-pyrrolo[2,3-d]pyrimidin-2-yl)amino)piperidin-1-yl)ethan-1-one [2592];
(S)-5-((5-(1-(2,2-difluoroethyl)-2-methyl-1H-benzo[d]imidazol-6-yl)-7H-pyrrolo[2,3-d]pyrimidin-2-yl)amino)-1-methylpiperidin-2-one [2593];
(R)-5-((5-(1-(2,2-difluoroethyl)-2-methyl-1H-benzo[d]imidazol-6-yl)-7H-pyrrolo[2,3-d]pyrimidin-2-yl)amino)-1-methylpiperidin-2-one [2594];
1-(2-((5-(1-(2,2-difluoroethyl)-2-methyl-1H-benzo[d]imidazol-6-yl)-7H-pyrrolo[2,3-d]pyrimidin-2-yl)amino)-7-azaspiro[3.5]nonan-7-yl)ethan-1-one [2595];
1-((3aR,5s,6aS)-5-((5-(1-(2,2-difluoroethyl)-2-methyl-1H-benzo[d]imidazol-6-yl)-7H-pyrrolo[2,3-d]pyrimidin-2-yl)amino)hexahydrocyclopenta[c]pyrrol-2(1H)-yl)ethan-1-one [2596];
1-((3aR,5r,6aS)-5-((5-(1-(2,2-difluoroethyl)-2-methyl-1H-benzo[d]imidazol-6-yl)-7H-pyrrolo[2,3-d]pyrimidin-2-yl)amino)hexahydrocyclopenta[c]pyrrol-2(1H)-yl)ethan-1-one [2597];
1-(7-((5-(1-(2,2-difluoroethyl)-2-methyl-1H-benzo[d]imidazol-6-yl)-7H-pyrrolo[2,3-d]pyrimidin-2-yl)amino)-2-azaspiro[3.5]nonan-2-yl)ethan-1-one [2598];
5-(1-(2,2-difluoroethyl)-2-methyl-1H-benzo[d]imidazol-6-yl)-N-(2-oxaspiro[3.5]nonan-7-yl)-7H-pyrrolo[2,3-d]pyrimidin-2-amine [2599];
5-(1-(2,2-difluoroethyl)-2-methyl-1H-benzo[d]imidazol-6-yl)-N-(1,4-dioxaspiro[4.5]decan-8-yl)-7H-pyrrolo[2,3-d]pyrimidin-2-amine [2600];
(1s,3s)-3-((5-(1-(2,2-difluoroethyl)-2-methyl-1H-benzo[d]imidazol-6-yl)-4-methoxy-7H-pyrrolo[2,3-d]pyrimidin-2-yl)amino)-N,N,1-trimethylcyclobutane-1-carboxamide [2601];
(1r,3r)-3-((5-(1-(2,2-difluoroethyl)-2-methyl-1H-benzo[d]imidazol-6-yl)-4-methoxy-7H-pyrrolo[2,3-d]pyrimidin-2-yl)amino)-N,N,1-trimethylcyclobutane-1-carboxamide [2602];
((1s,3s)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)(pyrrolidin-1-yl)methanone [2603];
((1r,3r)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)(pyrrolidin-1-yl)methanone [2604];
N-((1s,3s)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)acetamide [2605];
N-((1r,3r)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)acetamide [2606];
N-((1s,3s)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)propionamide [2607];
N-((1r,3r)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)propionamide [2608];
1-((1r,3r)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)pyrrolidin-2-one [2609];
1-((1s,3s)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutyl)pyrrolidin-2-one [2610];
(1s,3s)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutan-1-ol [2611];
(1r,3r)-3-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclobutan-1-ol [2612];
(1s,4s)-4-((5-(1-(2,2-difluoroethyl)-2-methyl-1H-benzo[d]imidazol-6-yl)-4-methoxy-7H-pyrrolo[2,3-d]pyrimidin-2-yl)amino)-N,N-dimethylcyclohexane-1-carboxamide [2613];
((1s,4s)-4-((5-(1-(2,2-difluoroethyl)-2-methyl-1H-benzo[d]imidazol-6-yl)-4-methoxy-7H-pyrrolo[2,3-d]pyrimidin-2-yl)amino)cyclohexyl)(pyrrolidin-1-yl)methanone [2614];
N-((1s,4s)-4-((5-(1-(2,2-difluoroethyl)-2-methyl-1H-benzo[d]imidazol-6-yl)-4-methoxy-7H-pyrrolo[2,3-d]pyrimidin-2-yl)amino)cyclohexypacetamide [2615];
N-((1r,4r)-4-((5-(1-(2,2-difluoroethyl)-2-methyl-1H-benzo[d]imidazol-6-yl)-4-methoxy-7H-pyrrolo[2,3-d]pyrimidin-2-yl)amino)cyclohexypacetamide [2616];
1-((1s,4s)-4-((5-(1-(2,2-difluoroethyl)-2-methyl-1H-benzo[d]imidazol-6-yl)-4-methoxy-7H-pyrrolo[2,3-d]pyrimidin-2-yl)amino)cyclohexyl)pyrrolidin-2-one [2617];
2-(((1s,4s)-4-((5-(1-(2,2-difluoroethyl)-2-methyl-1H-benzo[d]imidazol-6-yl)-4-methoxy-7H-pyrrolo[2,3-d]pyrimidin-2-yl)amino)cyclohexyl)oxy)ethan-1-ol [2618];
2-(((1r,4r)-4-((5-(1-(2,2-difluoroethyl)-2-methyl-1H-benzo[d]imidazol-6-yl)-4-methoxy-7H-pyrrolo[2,3-d]pyrimidin-2-yl)amino)cyclohexyl)oxy)ethan-1-ol [2619];
N-((1r,4r)-4-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylcyclohexypacetamide [2620];
(1r,4r)-4-((5-(1-(2,2-difluoroethyl)-2-methyl-1H-benzo[d]imidazol-6-yl)-4-(methoxy-d₃)-7H-pyrrolo[2,3-d]pyrimidin-2-yl)amino)-1-methylcyclohexan-1-ol [2621];
(1s,4s)-4-((5-(1-(2,2-difluoroethyl)-2-methyl-1H-benzo[d]imidazol-6-yl)-4-(methoxy-d₃)-7H-pyrrolo[2,3-d]pyrimidin-2-yl)amino)-1-methylcyclohexan-1-ol [2622];
(1s,4s)-4-((5-(1-(2,2-difluoroethyl)-2-methyl-1H-benzo[d]imidazol-6-yl)-4-methoxy-7H-pyrrolo[2,3-d]pyrimidin-2-yl)amino)-1-ethylcyclohexan-1-ol [2623];
1-(4-((5-(1-(2,2-difluoroethyl)-2-methyl-1H-benzo[d]imidazol-6-yl)-4-methoxy-7H-pyrrolo[2,3-d]pyrimidin-2-yl)amino)piperidin-1-yl)ethan-1-one [2624];
(S)-5-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylpiperidin-2-one [2625];
(R)-5-((5-(1-(2,2-difluoroethyl)-2-methyl-1H-benzo[d]imidazol-6-yl)-4-methoxy-7H-pyrrolo[2,3-d]pyrimidin-2-yl)amino)-1-methylpiperidin-2-one [2626];
1-(2-((5-(1-(2,2-difluoroethyl)-2-methyl-1H-benzo[d]imidazol-6-yl)-4-methoxy-7H-pyrrolo[2,3-d]pyrimidin-2-yl)amino)-7-azaspiro[3.5]nonan-7-yl)ethan-1-one [2627];
1-((3aR,5r,6aS)-5-((5-(1-(2,2-difluoroethyl)-2-methyl-1H-benzo[d]imidazol-6-yl)-4-methoxy-7H-pyrrolo[2,3-d]pyrimidin-2-yl)amino)hexahydrocyclopenta[c]pyrrol-2(1H)-yl)ethan-1-one [2628];
1-((3aR,5s,6aS)-5-((5-(1-(2,2-difluoroethyl)-2-methyl-1H-benzo[d]imidazol-6-yl)-4-methoxy-7H-pyrrolo[2,3-d]pyrimidin-2-yl)amino)hexahydrocyclopenta[c]pyrrol-2(1H)-yl)ethan-1-one [2629];
1-(7-((5-(1-(2,2-difluoroethyl)-2-methyl-1H-benzo[d]imidazol-6-yl)-4-methoxy-7H-pyrrolo[2,3-d]pyrimidin-2-yl)amino)-2-azaspiro[3.5]nonan-2-yl)ethan-1-one [2630];
5-(1-(2,2-difluoroethyl)-2-methyl-1H-benzo[d]imidazol-6-yl)-4-methoxy-N-(1,4-dioxaspiro[4.5]decan-8-yl)-7H-pyrrolo[2,3-d]pyrimidin-2-amine [2631];
(1s,3s)-3-((5-(1-(2,2-difluoroethyl)-4-fluoro-2-methyl-1H-benzo[d]imidazol-6-yl)-7H-pyrrolo[2,3-d]pyrimidin-2-yl)amino)-N,N,1-trimethylcyclobutane-1-carboxamide [2632];
3-((5-(1-(2,2-difluoroethyl)-4-fluoro-2-methyl-1H-benzo[d]imidazol-6-yl)-7H-pyrrolo[2,3-d]pyrimidin-2-yl)amino)-N,N,1-trimethylcyclobutane-1-carboxamide [2633];
(3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)(pyrrolidin-1-yl)methanone [2634];
((1s,3s)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)(pyrrolidin-1-yl)methanone [2635];
N-((1s,3s)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)acetamide [2636];
N-((1r,3r)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutypacetamide [2637];
N-((1s,3s)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)propionamide [2638];
N-((1r,3r)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)propionamide [2639];
1-((1r,3r)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)pyrrolidin-2-one [2640];
1-((1s,3s)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutyl)pyrrolidin-2-one [2641];
(1s,3s)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutan-1-ol [2642];
(1r,3r)-3-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclobutan-1-ol [2643];
(1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)-N,N-dimethylcyclohexane-1-carboxamide [2644];
((1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)cyclohexyl)(pyrrolidin-1-yl)methanone [2645];
N-((1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)cyclohexypacetamide [2646];
N-((1r,4r)-4-((5-(1-(2,2-difluoroethyl)-4-fluoro-2-methyl-1H-benzo[d]imidazol-6-yl)-7H-pyrrolo[2,3-d]pyrimidin-2-yl)amino)cyclohexypacetamide [2647];
1-((1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)cyclohexyl)pyrrolidin-2-one [2648];
2-(((1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)cyclohexyl)oxy)ethan-1-ol [2649];
2-(((1r,4r)-4-((5-(1-(2,2-difluoroethyl)-4-fluoro-2-methyl-1H-benzo[d]imidazol-6-yl)-7H-pyrrolo[2,3-d]pyrimidin-2-yl)amino)cyclohexyl)oxy)ethan-1-ol [2650];
N-((1r,4r)-4-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclohexypacetamide [2651];
(1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclohexan-1-ol [2652];
(1r,4r)-4-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylcyclohexan-1-ol [2653];
(1s,4s)-4-((5-(1-(2,2-difluoroethyl)-4-fluoro-2-methyl-1H-benzo[d]imidazol-6-yl)-7H-pyrrolo[2,3-d]pyrimidin-2-yl)amino)-1-ethylcyclohexan-1-ol [2654];
1-(4-((5-(1-(2,2-difluoroethyl)-4-fluoro-2-methyl-1H-benzo[d]imidazol-6-yl)-7H-pyrrolo[2,3-d]pyrimidin-2-yl)amino)piperidin-1-yl)ethan-1-one [2655];
(S)-5-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylpiperidin-2-one [2656];
(R)-5-((5-(1-(2,2-difluoroethyl)-4-fluoro-2-methyl-1H-benzo[d]imidazol-6-yl)-7H-pyrrolo[2,3-d]pyrimidin-2-yl)amino)-1-methylpiperidin-2-one [2657];
1-(2-((5-(1-(2,2-difluoroethyl)-4-fluoro-2-methyl-1H-benzo[d]imidazol-6-yl)-7H-pyrrolo[2,3-d]pyrimidin-2-yl)amino)-7-azaspiro[3.5]nonan-7-yl)ethan-1-one [2658];
1-((3aR,5s,6aS)-5-((5-(1-(2,2-difluoroethyl)-4-fluoro-2-methyl-1H-benzo[d]imidazol-6-yl)-7H-pyrrolo[2,3-d]pyrimidin-2-yl)amino)hexahydrocyclopenta[c]pyrrol-2(1H)-yl)ethan-1-one [2659];
1-((3aR,5r,6aS)-5-((5-(1-(2,2-difluoroethyl)-4-fluoro-2-methyl-1H-benzo[d]imidazol-6-yl)-7H-pyrrolo[2,3-d]pyrimidin-2-yl)amino)hexahydrocyclopenta[c]pyrrol-2(1H)-yl)ethan-1-one [2660];
1-(7-((5-(1-(2,2-difluoroethyl)-4-fluoro-2-methyl-1H-benzo[d]imidazol-6-yl)-7H-pyrrolo[2,3-d]pyrimidin-2-yl)amino)-2-azaspiro[3.5]nonan-2-yl)ethan-1-one [2661];
5-(1-(2,2-difluoroethyl)-4-fluoro-2-methyl-1H-benzo[d]imidazol-6-yl)-N-(2-oxaspiro[3.5]nonan-7-yl)-7H-pyrrolo[2,3-d]pyrimidin-2-amine [2662];
5-(1-(2,2-difluoroethyl)-4-fluoro-2-methyl-1H-benzo[d]imidazol-6-yl)-N-(1,4-dioxaspiro[4.5]decan-8-yl)-7H-pyrrolo[2,3-d]pyrimidin-2-amine [2663];
(1s,3s)-3-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-N,N,1-trimethylcyclobutane-1-carboxamide [2664];
3-((5-(1-(2,2-difluoroethyl)-4-fluoro -2-methyl-1H-benzo [d] imidazol -6-yl)-4-methoxy-7H-pyrrolo [2,3-d] pyrimidin-2-yl)amino)-N,N, 1-trimethylcyclobutane-1-carboxamide [2665];
((1 s , 3 s) -3-((5-(1-(2,2-difluoroethyl)-4-fluoro -2-methyl -1H-benzo [d] imidazol -6-yl)-4-methoxy-7H-pyrrolo [2,3-d] pyrimidin-2-yl)amino)-1-methylcyclobutyl)(pyrrolidin-1-yl)methanone [2666];
(3-((5-(1-(2,2-difluoroethyl)-4-fluoro -2-methyl -1H-benzo [ d] imidazol -6-yl) -4-methoxy-7H-pyrrolo [2,3-d] pyrimidin-2-yl)amino)-1-methylcyclobutyl)(pyrrolidin-1-yl)methanone [2667];
N-((1 s,3 s)-3-((5-(1-(2,2-difluoroethyl)-4-fluoro -2-methyl-1H-benzo [d] imidazol -6-yl)-4-methoxy-7H-pyrrolo [2,3-d] pyrimidin-2-yl)amino)-1-methylcyclobutyl)acetamide [2668];
N-((1 r,3 r)-3-((5-(1-(2,2-difluoroethyl)-4-fluoro -2-methyl-1H-benzo [d] imidazol -6-yl)-4-methoxy-7H-pyrrolo [2,3-d] pyrimidin-2-yl)amino)-1-methylcyclobutyl)acetamide [2669];
N-((1 s,3 s)-3-((5-(1-(2,2-difluoroethyl)-4-fluoro -2-methyl-1H-benzo [d] imidazol -6-yl)-4-methoxy-7H-pyrrolo [2,3-d] pyrimidin-2-yl)amino)-1-methylcyclobutyl)propionamide [2670];
N-((1 r,3 r)-3-((5-(1-(2,2-difluoroethyl)-4-fluoro -2-methyl-1H-benzo [d] imidazol -6-yl)-4-methoxy-7H-pyrrolo [2,3-d] pyrimidin-2-yl)amino)-1-methylcyclobutyl)propionamide [2671];
1-((1 r,3 r)-3-((5-(1-(2,2-difluoroethyl)-4-fluoro -2-methyl -1H-benzo [d] imidazol -6-yl)-4-methoxy-7H-pyrrolo [2,3-d] pyrimidin-2-yl)amino)-1-methylcyclobutyl)pyrrolidin-2-one [2672];
1-((1 s, 3 s)-3-((5-(1-(2,2-difluoroethyl)-4-fluoro -2-methyl-1H-benzo [d] imidazol -6-yl)-4-methoxy-7H-pyrrolo [2,3-d] pyrimidin-2-yl)amino)-1-methylcyclobutyl)pyrrolidin-2-one [2673];
(1 s,3 s) -3-((5-(1-(2,2-difluoroethyl) -4-fluoro -2-methyl -1H-benzo [d] imidazol -6-yl)-4-methoxy-7H-pyrrolo [2,3-d] pyrimidin-2-yl)amino)-1-methylcyclobutan-1-ol [2674];
(1 r, 3 r) -3-((5-(1-(2,2-difluoroethyl) -4-fluoro -2-methyl -1H-benzo [d] imidazol -6-yl)-4-methoxy-7H-pyrrolo [2,3-d] pyrimidin-2-yl)amino)-1-methylcyclobutan-1-ol [2675];
(1 s ,4 s) -4-((5-(1-(2,2-difluoroethyl) -4-fluoro -2-methyl -1H-benzo [d] imidazol -6-yl)-4-methoxy-7H-pyrrolo [2,3-d] pyrimidin-2-yl)amino)-N,N-dimethylcyclohexane-1-carboxamide [2676];
((1 s ,4 s) -4-((5-(1-(2,2-difluoroethyl)-4-fluoro -2-methyl -1H-benzo [d] imidazol -6-yl)-4-methoxy-7H-pyrrolo [2,3-d] pyrimidin-2-yl)amino)cyclohexyl)(pyrrolidin-1-yl)methanone [2677];
N-((1 s,4s)-4-((5-(1-(2,2-difluoroethyl)-4-fluoro -2-methyl-1H-benzo [d] imidazol -6-yl)-4-methoxy-7H-pyrrolo[2,3-d]pyrimidin-2-yl)amino)cyclohexypacetamide [2678];
N-((1 r,4r)-4-((5-(1-(2,2-difluoroethyl)-4-fluoro -2-methyl-1H-benzo [d] imidazol -6-yl)-4-methoxy-7H-pyrrolo[2,3-d]pyrimidin-2-yl)amino)cyclohexypacetamide [2679];
1-((1 s,4s)-4-((5-(1-(2,2-difluoroethyl)-4-fluoro -2-methyl-1H-benzo [d] imidazol -6-yl)-4-methoxy-7H-pyrrolo[2,3-d]pyrimidin-2-yl)amino)cyclohexyl)pyrrolidin-2-one [2680];
2-(((1s,4s)-4-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)cyclohexyl)oxy)ethan-1-ol [2681];
2-(((1r,4r)-4-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)cyclohexyl)oxy)ethan-1-ol [2682];
N-((1r,4r)-4-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-1-methylcyclohexypacetamide [2683];
(1r,4r)-4-((5-(1-(2,2-difluoroethyl)-4-fluoro-2-methyl-1H-benzo[d]imidazol-6-yl)-4-(methoxy-d₃)-7H-pyrrolo[2,3-d]pyrimidin-2-yl)amino)-1-methylcyclohexan-1-ol [2684];
(1s,4s)-4-((5-(1-(2,2-difluoroethyl)-4-fluoro-2-methyl-1H-benzo[d]imidazol-6-yl)-4-(methoxy-d₃)-7H-pyrrolo[2,3-d]pyrimidin-2-yl)amino)-1-methylcyclohexan-1-ol [2685];
(1s,4s)-4-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-1-ethylcyclohexan-1-ol [2686];
1-(4-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)piperidin-1-yl)ethan-1-one [2687];
(R)-5-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-1-methylpiperidin-2-one [2688];
(S)-5-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-1-methylpiperidin-2-one [2689];
1-(2-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-7-azaspiro[3.5]nonan-7-yl)ethan-1-one [2690];
1-((3aR,5s,6aS)-5-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)hexahydrocyclopenta[c]pyrrol-2(1H)-yl)ethan-1-one [2691];
1-((3aR,5r,6aS)-5-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)hexahydrocyclopenta[c]pyrrol-2(1H)-yl)ethan-1-one [2692];
1-(7-((5-(1-(2,2-difluoroethyl)-4-fluoro-2-methyl-1H-benzo[d]imidazol-6-yl)-4-methoxy-7H-pyrrolo[2,3-d]pyrimidin-2-yl)amino)-2-azaspiro[3.5]nonan-2-yl)ethan-1-one [2693];
5-(1-(2,2-difluoroethyl)-4-fluoro-2-methyl-1H-benzo[d]imidazol-6-yl)-4-methoxy-N-(1,4-dioxaspirop.51decan-8-yl)-7H-pyrrolo[2,3-d]pyrimidin-2-amine [2694];
(1s,3s)-3-((5-(1-(2,2-difluoroethyl)-2-methyl-1H-imidazo[4,5-b]pyridin-6-yl)-7H-pyrrolo[2,3-d]pyrimidin-2-yl)amino)-N,N,1-trimethylcyclobutane-1-carboxamide [2695];
(1r,3r)-3-((5-(1-(2,2-difluoroethyl)-2-methyl-1H-imidazo[4,5-b]pyridin-6-yl)-7H-pyrrolo[2,3-d]pyrimidin-2-yl)amino)-N,N,1-trimethylcyclobutane-1-carboxamide [2696];
((1r,3r)-3-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclobutyl)(pyrrolidin-1-yl)methanone [2697];
((1s,3s)-3-((5-(1-(2,2-difluoroethyl)-2-methyl-1H-imidazo[4,5-b]pyridin-6-yl)-7H-pyrrolo [2,3-d]pyrimidin-2-yl)amino)-1-methylcyclobutyl)(pyrrolidin-1-yl)methanone [2698];
N-((1s,3s)-3-((5-(1-(2,2-difluoroethyl)-2-methyl-1H-imidazo [4,5-b]pyridin-6-yl)-7H-pyrrolo [2,3-d]pyrimidin-2-yl)amino)-1-methylcyclobutyl)acetamide [2699];
N-((1r,3r)-3-((5-(1-(2,2-difluoroethyl)-2-methyl-1H-imidazo [4,5-b]pyridin-6-yl)-7H-pyrrolo [2,3-d]pyrimidin-2-yl)amino)-1-methylcyclobutyl)acetamide [2700];
N-(3-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclobutyl)propionamide [2701];
N-((1r,3r)-3-((5-(1-(2,2-difluoroethyl)-2-methyl-1H-imidazo [4,5-b]pyridin-6-yl)-7H-pyrrolo [2,3-d]pyrimidin-2-yl)amino)-1-methylcyclobutyl)propionamide [2702];
1-((1r,3r)-3-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclobutyl)pyrrolidin-2-one [2703];
1-((1s,3s)-3-((5-(1-(2,2-difluoroethyl)-2-methyl-1H-imidazo [4,5-b]pyridin-6-yl)-7H-pyrrolo [2,3-d]pyrimidin-2-yl)amino)-1-methylcyclobutyl)pyrrolidin-2-one [2704];
(1s,3s)-3-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclobutan-1-ol [2705];
(1r,3r)-3-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclobutan-1-ol [2706];
(1s,4s)-4-((5-(1-(2,2-difluoroethyl)-2-methyl-1H-imidazo [4,5-b] pyridin-6-yl)-7H-pyrrolo [2,3-d]pyrimidin-2-yl)amino)-N,N-dimethylcyclohexane-1-carboxamide [2707];
((1s,4s)-4-((5-(1-(2,2-difluoroethyl)-2-methyl-1H-imidazo[4,5-b]pyridin-6-yl)-7H-pyrrolo [2,3-d]pyrimidin-2-yl)amino)cyclohexyl)(pyrrolidin-1-yl)methanone [2708];
N-((1s,4s)-4-((5-(1-(2,2-difluoroethyl)-2-methyl-1H-imidazo [4,5-b]pyridin-6-yl)-7H-pyrrolo [2,3-d]pyrimidin-2-yl)amino)cyclohexypacetamide [2709];
N-((1r,40-4-45-(1-(2,2-difluoroethyl)-2-methyl-1H-imidazo [4,5-b]pyridin-6-yl)-7H-pyrrolo [2,3-d]pyrimidin-2-yl)amino)cyclohexypacetamide [2710];
1-((1s,4s)-4-((5-(1-(2,2-difluoroethyl)-2-methyl-1H-imidazo [4,5-b]pyridin-6-yl)-7H-pyrrolo [2,3-d]pyrimidin-2-yl)amino)cyclohexyl)pyrrolidin-2-one [2711];
5-(1-(2,2-difluoroethyl)-2-methyl-1H-imidazo[4,5-b]pyridin-6-yl)-N-((1s,4s)-4-methoxycyclohexyl)-7H-pyrrolo [2,3-d]pyrimidin-2-amine [2712];
2-(((1s,4s)-4-((5-(1-(2,2-difluoroethyl)-2-methyl-1H-imidazo [4,5-b]pyridin-6-yl)-7H-pyrrolo [2,3-d]pyrimidin-2-yl)amino)cyclohexyl)oxy)e than-1-ol [2713];
2-(((1 r,40-4-45-(1-(2,2-difluoroethyl)-2-methyl-1H-imidazo [4,5-b]pyridin-6-yl)-7H-pyrrolo [2,3-d]pyrimidin-2-yl)amino)cyclohexyl)oxy)ethan-1-ol [2714];
N-((1 r,40-4-45-(1-(2,2-difluoroethyl)-2-methyl-1H-imidazo [4,5-b]pyridin-6-yl)-7H-pyrrolo [2,3-d]pyrimidin-2-yl)amino)-1-methylcyclohexyl)acetamide [2715];
(1 r,4r)-4-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclohexan-1-ol [2716];
(1 s,4 s)-4-((5-(1-(2,2-difluoroethyl)-2-methyl-1H-imidazo [4,5-b] pyridin-6-yl)-7H-pyrrolo [2,3-d]pyrimidin-2-yl)amino)-1-methylcyclohexan-1-ol [2717];
(1 s,4 s)-4-((5-(1-(2,2-difluoroethyl)-2-methyl-1H-imidazo [4,5-b] pyridin-6-yl)-7H-pyrrolo [2,3-d]pyrimidin-2-yl)amino)-1-ethylcyclohexan-1-ol [2718];
1-(4-((5-(1-(2,2-difluoroethyl)-2-methyl-1H-imidazo [4,5-b]pyridin-6-yl)-7H-pyrrolo [2,3-d]pyrimidin-2-yl)amino)piperidin-1-yl)ethan-1-one [2719];
(S)-5-((5-(1-(2,2-difluoroethyl)-2-methyl-1H-imidazo [4,5-b]pyridin-6-yl)-7H-pyrrolo [2,3-d]pyrimidin-2-yl)amino)-1-methylpiperidin-2-one [2720];
(R)-5-((5-(1-(2,2-difluoroethyl)-2-methyl-1H-imidazo [4,5-b]pyridin-6-yl)-7H-pyrrolo [2,3-d]pyrimidin-2-yl)amino)-1-methylpiperidin-2-one [2721];
1-(2-((5-(1-(2,2-difluoroethyl)-2-methyl-1H-imidazo [4,5-b] pyridin-6-yl)-7H-pyrrolo [2,3-d]pyrimidin-2-yl)amino)-7-azaspiro [3 .5]nonan-7-yl)ethan-1-one [2722];
1-((3 aR,5 s,6aS)-5-((5-(1-(2,2-difluoroethyl)-2-methyl-1H-imidazo [4,5-b] pyridin-6-yl)-7H-pyrrolo [2,3-d]pyrimidin-2-yl)amino)hexahydrocyclopenta[c]pyrrol-2(1H)-yl)ethan-1-one [2723];
1-((3 aR,5 r,6aS)-5-((5-(1-(2,2-difluoroethyl)-2-methyl-1H-imidazo [4,5-b]pyridin-6-yl)-7H-pyrrolo [2,3-d]pyrimidin-2-yl)amino)hexahydrocyclopenta[c]pyrrol-2(1H)-yl)ethan-1-one [2724];
1-(7-((5-(1-(2,2-difluoroethyl)-2-methyl-1H-imidazo [4,5-b] pyridin-6-yl)-7H-pyrrolo [2,3-d]pyrimidin-2-yl)amino)-2-azaspiro [3 .5]nonan-2-yl)ethan-1-one [2725];
5-(1-(2,2-difluoroethyl)-2-methyl-1H-imidazo [4,5-b] pyridin-6-yl)-N-(2-oxaspiro [3 .5]nonan-7-yl)-7H-pyrrolo [2,3-d]pyrimidin-2-amine [2726];
5-(1-(2,2-difluoroethyl)-2-methyl-1H-imidazo [4,5-b]pyridin-6-yl)-N-(1,4-dioxaspiro [4 .5] de can-8-yl)-7H-pyrrolo [2,3-d]pyrimidin-2-amine [2727];
(1 s,3 s)-3-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-N,N, 1-trimethylcyclobutane-1-carboxamide [2728];
(1 r,3 r)-3-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-N,N, 1-trimethylcyclobutane-1-carboxamide [2729];
((1r,3r)-3-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-1-methylcyclobutyl)(pyrrolidin-1-yl)methanone [2730];
((1s,3s)-3-((5-(1-(2,2-difluoroethyl)-2-methyl-1H-imidazo[4,5-b]pyridin-6-yl)-4-methoxy-7H-pyrrolo [2,3-d]pyrimidin-2-yl)amino)-1-methylcyclobutyl)(pyrrolidin-1-yl)methanone [2731];
N-((1s,3s)-3-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylcyclobutyl)acetamide [2732];
N-((1r,3r)-3-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylcyclobutyl)acetamide [2733];
N-((1s,3s)-3-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylcyclobutyl)propionamide [2734];
N-((1r,3r)-3-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylcyclobutyl)propionamide [2735];
1-((1r,3r)-3-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-1-methylcyclobutyl)pyrrolidin-2-one [2736];
1-((1s,3s)-3-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylcyclobutyl)pyrrolidin-2-one [2737];
(1s,3s)-3-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-1-methylcyclobutan-1-ol [2738];
(1r,3r)-3-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-1-methylcyclobutan-1-ol [2739];
(1s,4s)-4-((5-(1-(2,2-difluoroethyl)-2-methyl-1H-imidazo [4,5-b] pyridin-6-yl)-4-methoxy-7H-pyrrolo [2,3-d]pyrimidin-2-yl)amino)-N,N-dimethylcyclohexane-1-carboxamide [2740];
((1s,4s)-4-((5-(1-(2,2-difluoroethyl)-2-methyl-1H-imidazo[4,5-b]pyridin-6-yl)-4-methoxy-7H-pyrrolo [2,3-d]pyrimidin-2-yl)amino)cyclohexyl)(pyrrolidin-1-yl)methanone [2741];
N-((1r,40-4-45-(1-(2,2-difluoroethyl)-2-methyl-1H-imidazo [4,5-b]pyridin-6-yl)-4-methoxy-7H-pyrrolo [2,3-d]pyrimidin-2-yl)amino)cyclohexypacetamide [2742];
N-((1s,4s)-4-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)cyclohexypacetamide [2743];
1-((1s,4s)-4-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)cyclohexyl)pyrrolidin-2-one [2744];
5-(1-(2,2-difluoroethyl)-2-methyl-1H-imidazo[4,5-b]pyridin-6-yl)-4-methoxy-N-((1s,4s)-4-methoxycyclohexyl)-7H-pyrrolo [2,3-d]pyrimidin-2-amine [2745];
2-(((1r,40-4-45-(1-(2,2-difluoroethyl)-2-methyl-1H-imidazo [4,5-b]pyridin-6-yl)-4-methoxy-7H-pyrrolo [2,3-d]pyrimidin-2-yl)amino)cyclohexyl)oxy)ethan-1-ol [2746];
2-(((1s,4s)-4-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)cyclohexyl)oxy)ethan-1-ol [2747];
N-((1r,40-4-45-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylcyclohexypacetamide [2748];
(1s,4s)-4-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-ethylcyclohexan-1-ol [2749];
(1s,4s)-4-((5-(1-(2,2-difluoroethyl)-2-methyl-1H-imidazo [4,5-b]pyridin-6-yl)-4-(methoxy-d₃)-7H-pyrrolo [2,3-d]pyrimidin-2-yl)amino)-1-methylcyclohexan-1-ol [2750];
(1r,40-4-45-(1-(2,2-difluoroethyl)-2-methyl-1H-imidazo [4,5-b]pyridin-6-yl)-4-(methoxy-d₃)-7H-pyrrolo [2,3-d]pyrimidin-2-yl)amino)-1-methylcyclohexan-1-ol [2751];
1-(4-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)piperidin-1-yl)ethan-1-one [2752];
(S)-5-((5-(1-(2,2-difluoroethyl)-2-methyl-1H-imidazo[4,5-b]pyridin-6-yl)-4-methoxy-7H-pyrrolo[2,3-d]pyrimidin-2-yl)amino)-1-methylpiperidin-2-one [2753];
(R)-5-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-1-methylpiperidin-2-one [2754];
1-(2-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-7-azaspiro [3.5]nonan-7-yl)ethan-1-one [2755];
1-((3aR,5r,6aS)-5-((5-(1-(2,2-difluoroethyl)-2-methyl-1H-imidazo[4,5-b]pyridin-6-yl)-4-methoxy-7H-pyrrolo[2,3-d]pyrimidin-2-yl)amino)hexahydrocyclopenta[c]pyrrol-2(1H)-yl)ethan-1-one [2756];
1-((3aR,5 s,6aS)-5-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)hexahydrocyclopenta[c]pyrrol-2(1H)-yl)ethan-1-one [2757];
1-(7-((5-(1-(2,2-difluoroethyl)-2-methyl-1H-imidazo [4,5-b]pyridin-6-yl)-4-methoxy-7H-pyrrolo [2,3-d]pyrimidin-2-yl)amino)-2-azaspiro [3.5]nonan-2-yl)ethan-1-one [2758];
5-(1-(2,2-difluoroethyl)-2-methyl-1H-imidazo[4,5-b]pyridin-6-yl)-4-methoxy-N-(2-oxaspiro [3.5]nonan-7-yl)-7H-pyrrolo [2,3-d]pyrimidin-2-amine [2759];
5-(1-(2,2-difluoroethyl)-2-methyl-1H-imidazo[4,5-b]pyridin-6-yl)-4-methoxy-N-(1,4-dioxaspiro [4.5]decan-8-yl)-7H-pyrrolo [2,3-d]pyrimidin-2-amine [2760];
(1s,3 s)-3-45-(3-(2,2-difluoroethyl)-2-methyl-3H-imidazo [4,5-b]pyridin-5-yl)-7H-pyrrolo [2,3-d]pyrimidin-2-yl)amino)-N,N,1-trimethylcyclobutane-1-carboxamide [2761];
(1r,30-3-45-(3-(2,2-difluoroethyl)-2-methyl-3H-imidazo [4,5-b]pyridin-5-yl)-7H-pyrrolo [2,3-d]pyrimidin-2-yl)amino)-N,N,1-trimethylcyclobutane-1-carboxamide [2762];
((1 s,3 s)-3-((5-(3-(2,2-difluoroethyl)-2-methyl-3H-imidazo [4,5-b]pyridin-5-yl)-7H-pyrrolo [2,3-d]pyrimidin-2-yl)amino)-1-methylcyclobutyl)(pyrrolidin-1-yl)methanone [2763];
((1 r,3 r)-3-45-(3-(2,2-difluoroethyl)-2-methyl-3H-imidazo [4,5-b] pyridin-5-yl)-7H-pyrrolo [2,3-d]pyrimidin-2-yl)amino)-1-methylcyclobutyl)(pyrrolidin-1-yl)methanone [2764];
N-((1 r,3 r)-3-45-(3-(2,2-difluoroethyl)-2-methyl-3H-imidazo [4,5-b]pyridin-5-yl)-7H-pyrrolo [2,3-d]pyrimidin-2-yl)amino)-1-methylcyclobutyl)acetamide [2765];
N-((1 s,3 s)-3-45-(3-(2,2-difluoroethyl)-2-methyl-3H-imidazo [4,5-b]pyridin-5-yl)-7H-pyrrolo [2,3-d]pyrimidin-2-yl)amino)-1-methylcyclobutyl)acetamide [2766];
N-(3-((5-(3-(2,2-difluoroethyl)-2-methyl-3H-imidazo [4,5-b] pyridin-5-yl)-7H-pyrrolo [2,3-d]pyrimidin-2-yl)amino)-1-methylcyclobutyl)propionamide [2767];
N-((1 r,3 r)-3-45-(3-(2,2-difluoroethyl)-2-methyl-3H-imidazo [4,5-b]pyridin-5-yl)-7H-pyrrolo [2,3-d]pyrimidin-2-yl)amino)-1-methylcyclobutyl)propionamide [2768];
1-((1 r,3 r)-3-45-(3-(2,2-difluoroethyl)-2-methyl-3H-imidazo [4,5-b] pyridin-5-yl)-7H-pyrrolo [2,3-d]pyrimidin-2-yl)amino)-1-methylcyclobutyl)pyrrolidin-2-one [2769];
1-((1 s,3 s)-3-45-(3-(2,2-difluoroethyl)-2-methyl-3H-imidazo [4,5-b]pyridin-5-yl)-7H-pyrrolo [2,3-d]pyrimidin-2-yl)amino)-1-methylcyclobutyl)pyrrolidin-2-one [2770];
(1 r,3 r)-3-45-(3-(2,2-difluoroethyl)-2-methyl-3H-imidazo [4,5-b] pyridin-5-yl)-7H-pyrrolo [2,3-d]pyrimidin-2-yl)amino)-1-methylcyclobutan-1-ol [2771];
(1 s,3 s)-3-45-(3-(2,2-difluoroethyl)-2-methyl-3H-imidazo [4,5-b] pyridin-5-yl)-7H-pyrrolo [2,3-d]pyrimidin-2-yl)amino)-1-methylcyclobutan-1-ol [2772];
(1 s,4s)-4-45-(3-(2,2-difluoroethyl)-2-methyl-3H-imidazo [4,5-b] pyridin-5-yl)-7H-pyrrolo [2,3-d]pyrimidin-2-yl)amino)-N,N-dimethylcyclohexane-1-carboxamide [2773];
((1 s,4s)-4-45-(3-(2,2-difluoroethyl)-2-methyl-3H-imidazo [4,5-b]pyridin-5-yl)-7H-pyrrolo [2,3-d]pyrimidin-2-yl)amino)cyclohexyl)(pyrrolidin-1-yl)methanone [2774];
N-((1 s,4s)-4-45-(3-(2,2-difluoroethyl)-2-methyl-3H-imidazo [4,5-b]pyridin-5-yl)-7H-pyrrolo [2,3-d]pyrimidin-2-yl)amino)cyclohexypacetamide [2775];
N-((1 r,40-4-45-(3-(2,2-difluoroethyl)-2-methyl-3H-imidazo [4,5-b]pyridin-5-yl)-7H-pyrrolo [2,3-d]pyrimidin-2-yl)amino)cyclohexypacetamide [2776];
1-((1 s,4s)-4-45-(3-(2,2-difluoroethyl)-2-methyl-3H-imidazo [4,5-b]pyridin-5-yl)-7H-pyrrolo [2,3-d]pyrimidin-2-yl)amino)cyclohexyl)pyrrolidin-2-one [2777];
5-(3-(2,2-difluoroethyl)-2-methyl-3H-imidazo [4,5-b] pyridin-5-yl)-N-((1 s,4s)-4-methoxycyclohexyl)-7H-pyrrolo [2,3-d]pyrimidin-2-amine [2778];
2-(((1 r,40-4-45-(3-(2,2-difluoroethyl)-2-methyl-3H-imidazo [4,5-b]pyridin-5-yl)-7H-pyrrolo [2,3-d]pyrimidin-2-yl)amino)cyclohexypoxy)e than-1-ol [2779];
2-(((1s,4 s)-4-45-(3-(2,2-difluoroethyl)-2-methyl-3H-imidazo [4,5-b]pyridin-5-yl)-7H-pyrrolo [2,3-d]pyrimidin-2-yl)amino)cyclohexyl)oxy)ethan-1-ol [2780];
N-((1r,40-4-45-(3-(2,2-difluoroethyl)-2-methyl-3H-imidazo [4,5-b]pyridin-5-yl)-7H-pyrrolo [2,3-d]pyrimidin-2-yl)amino)-1-methylcyclohexyl)acetamide [2781];
(1r,4r) -44(5-(3-(2,2-difluoroethyl)-2-methyl-3H-imidazo [4,5-1)] pyridin-5-yl)-7H-pyrrolo [2,3-d]pyrimidin-2-yl)amino)-1-methylcyclohexan-1-ol [2782];
(1s,4 s)-4-45-(3-(2,2-difluoroethyl)-2-methyl-3H-imidazo [4,5-b] pyridin-5-yl)-7H-pyrrolo [2,3-d]pyrimidin-2-yl)amino)-1-ethylcyclohexan-1-ol [2783];
1444(5-(3-(2,2-difluoroethyl)-2-methyl-3H-imidazo [4,5-b] pyridin-5-yl)-7H-pyrrolo [2,3-d]pyrimidin-2-yl)amino)piperidin-1-yl)ethan-1-one [2784];
(S)-5-((5-(3-(2,2-difluoroethyl)-2-methyl-3H-imidazo [4,5-b]pyridin-5-yl)-7H-pyrrolo [2,3-d]pyrimidin-2-yl)amino)-1-methylpiperidin-2-one [2785];
(R)-5-((5-(3-(2,2-difluoroethyl)-2-methyl-3H-imidazo [4,5-b]pyridin-5-yl)-7H-pyrrolo [2,3-d]pyrimidin-2-yl)amino)-1-methylpiperidin-2-one [2786];
1424(5-(3-(2,2-difluoroethyl)-2-methyl-3H-imidazo [4,5-b] pyridin-5-yl)-7H-pyrrolo [2,3-d]pyrimidin-2-yl)amino)-7-azaspiro [3 .5]nonan-7-yl)ethan-1-one [2787];
1-((3aR,5 s,6aS)-5-45-(3-(2,2-difluoro ethyl)-2-methyl-3H-imidazo [4,5-b] pyridin-5-yl)-7H-pyrrolo [2,3-d]pyrimidin-2-yl)amino)hexahydrocyclopenta [c]pyrrol-2 (1H)-yl)ethan-1-one [2788];
1-((3aR,5r,6aS)-5-45-(3-(2,2-difluoroethyl)-2-methyl-3H-imidazo [4,5-b]pyridin-5-yl)-7H-pyrrolo [2,3-d]pyrimidin-2-yl)amino)hexahydrocyclopenta [c]pyrrol-2 (1H)-yl)ethan-1-one [2789];
1474(5-(3-(2,2-difluoroethyl)-2-methyl-3H-imidazo [4,5-b] pyridin-5-yl)-7H-pyrrolo [2,3-d]pyrimidin-2-yl)amino)-2-azaspiro [3 .5]nonan-2-yl)ethan-1-one [2790];
5-(3-(2,2-difluoroethyl)-2-methyl-3H-imidazo [4,5-b] pyridin-5-yl)-N-(2-oxaspiro [3 .5]nonan-7-yl)-7H-pyrrolo [2,3-d] pyrimidin-2-amine [2791];
5-(3-(2,2-difluoroethyl)-2-methyl-3H-imidazo [4,5-b] pyridin-5-yl)-N-(1,4-dioxaspiro [4 .5] de can-8-yl)-7H-pyrrolo [2,3-d] pyrimidin-2-amine [2792];
N²-(4,4-difluorocyclohexyl)-5-(3-(2,2-difluoroethyl)-2-methyl-3H-imidazo [4,5-b]pyridin-5-yl)-N⁴-methyl-7H-pyrrolo [2,3-d] pyrimidine-2,4-diamine [2793];
(1r,4r) -44(5-(3-(2,2-difluoroethyl)-2-methyl-3H-imidazo [4,5-b] pyridin-5-yl)-4-(methylamino)-7H-pyrrolo [2,3-d]pyrimidin-2-yl)amino)-N,N-dimethylcyclohexane-1-carboxamide [2794];
(1s,4 s)-4-45-(3-(2,2-difluoroethyl)-2-methyl-3H-imidazo [4,5-b] pyridin-5-yl)-4-(methylamino)-7H-pyrrolo [2,3-d]pyrimidin-2-yl)amino)-N,N-dimethylcyclohexane-1-carboxamide [2795];
((1r,40-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)cyclohexyl)(pyrrolidin-1-yl)methanone [2796];
((1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)cyclohexyl)(pyrrolidin-1-yl)methanone [2797];
N-((1r,40-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)cyclohexypacetamide [2798];
N-((1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)cyclohexypacetamide [2799];
1-((1r,40-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)cyclohexyl)pyrrolidin-2-one [2800];
1-((1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)cyclohexyl)pyrrolidin-2-one [2801];
5-(3-(2,2-difluoroethyl)-2-methyl-3H-imidazo [4,5-b] pyridin-5-yl)-N²-((1s,4s)-4-methoxycyclohexyl)-N⁴-methyl-7H-pyrrolo[2,3-d]pyrimidine-2,4-diamine [2802];
5-(3-(2,2-difluoroethyl)-2-methyl-3H-imidazo [4,5-b] pyridin-5-yl)-N²-((1s,4s)-4-(difluoromethoxy)cyclohexyl)-N⁴-methyl-7H-pyrrolo[2,3-d]pyrimidine-2,4-diamine [2803];
5-(3-(2,2-difluoroethyl)-2-methyl-3H-imidazo [4,5-b] pyridin-5-yl)-N²-41r,4r) -4-(difluoromethoxy)cyclohexyl)-N⁴-methyl-7H-pyrrolo[2,3-d]pyrimidine-2,4-diamine [2804];
(1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-methylcyclohexan-1-ol [2805];
(1r,40-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-methylcyclohexan-1-ol [2806];
N-((1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-methylcyclohexypacetamide [2807];
N-((1r,40-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-methylcyclohexypacetamide [2808];
1-((1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-methylcyclohexyl)pyrrolidin-2-one [2809];
1-((1r,40-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-methylcyclohexyl)pyrrolidin-2-one [2810];
5-(3-(2,2-difluoroethyl)-2-methyl-3H-imidazo [4,5-b] pyridin-5-yl)-N²-((3R,4S)-3-fluoro-1-methylpiperidin-4-yl)-N⁴-methyl-7H-pyrrolo [2,3-d]pyrimidine-2,4-diamine [2811];
5-(3-(2,2-difluoroethyl)-2-methyl-3H-imidazo [4,5-b] pyridin-5-yl)-N²-((3 S,4R)-3-fluoro-1-methylpiperidin-4-yl)-N⁴-methyl-7H-pyrrolo [2,3-d]pyrimidine-2,4-diamine [2812];
5-(3-(2,2-difluoroethyl)-2-methyl-3H-imidazo [4,5-b] pyridin-5-yl)-N²-((3 S,4S)-3-fluoro-1-methylpiperidin-4-yl)-N⁴-methyl-7H-pyrrolo [2,3-d]pyrimidine-2,4-diamine [2813];
5-(3-(2,2-difluoroethyl)-2-methyl-3H-imidazo [4,5-b] pyridin-5-yl)-N²-((3R,4R)-3-fluoro-1-methylpiperidin-4-yl)-N⁴-methyl-7H-pyrrolo [2,3-d]pyrimidine-2,4-diamine [2814];
1-(7-45-(3-(2,2-difluoroethyl)-2-methyl-3H-imidazo [4,5-b] pyridin-5-yl)-4-(methylamino)-7H-pyrrolo [2,3-d]pyrimidin-2-yl)amino)-2-azaspiro [3 . 5 ]nonan-2-yl)ethan-1-one [2815];
5-(3-(2,2-difluoroethyl)-2-methyl-3H-imidazo [4,5-b] pyridin-5-yl)-N⁴-methyl-N²-(2-oxaspiro [3 . 5 ]nonan-7-yl)-7H-pyrrolo [2,3-d]pyrimidine-2,4-diamine [2816];
(R)-5-(3-(2,2-difluoroethyl)-2-methyl-3H-imidazo [4,5-b]pyridin-5-yl)-2-methoxy-N-(1, 1, 1-trifluoropropan-2-yl)-7H-pyrrolo [2,3-d]pyrimidin-4-amine [2817];
(1 s,3 s)-3-((5-(1-(2,2-difluoroethyl)-2-methyl-1H-imidazo [4,5-b] pyrazin-6-yl)-7H-pyrrolo [2,3-d]pyrimidin-2-yl)amino)-N,N, 1-trimethylcyclobutane-1-carboxamide [2818];
(1 r,3 r)-3-((5-(1-(2,2-difluoroethyl)-2-methyl-1H-imidazo [4,5-b] pyrazin-6-yl)-7H-pyrrolo [2,3-d]pyrimidin-2-yl)amino)-N,N, 1-trimethylcyclobutane-1-carboxamide [2819];
((1 r,3 r)-3-((5-(1-(2,2-difluoroethyl)-2-methyl-1H-imidazo [4,5-b] pyrazin-6-yl)-7H-pyrrolo [2,3-d]pyrimidin-2-yl)amino)-1-methylcyclobutyl)(pyrrolidin-1-yl)methanone [2820];
((1 s,3 s)-3-((5-(1-(2,2-difluoroethyl)-2-methyl-1H-imidazo [4,5-b]pyrazin-6-yl)-7H-pyrrolo [2,3-d]pyrimidin-2-yl)amino)-1-methylcyclobutyl)(pyrrolidin-1-yl)methanone [2821];
N-((1 s,3 s)-3-((5-(1-(2,2-difluoroethyl)-2-methyl-1H-imidazo [4,5-b]pyrazin-6-yl)-7H-pyrrolo [2,3-d]pyrimidin-2-yl)amino)-1-methylcyclobutypacetamide [2822];
N-((1 r,3 r)-3-((5-(1-(2,2-difluoroethyl)-2-methyl-1H-imidazo [4,5-b]pyrazin-6-yl)-7H-pyrrolo [2,3-d]pyrimidin-2-yl)amino)-1-methylcyclobutyl)acetamide [2823];
N-(3-((5-(1-(2,2-difluoroethyl)-2-methyl-1H-imidazo [4,5-b] pyrazin-6-yl)-7H-pyrrolo [2,3-d]pyrimidin-2-yl)amino)-1-methylcyclobutyl)propionamide [2824];
N-((1 r,3 r)-3-((5-(1-(2,2-difluoroethyl)-2-methyl-1H-imidazo [4,5-b]pyrazin-6-yl)-7H-pyrrolo [2,3-d]pyrimidin-2-yl)amino)-1-methylcyclobutyl)propionamide [2825];
1-((1 r,3 r)-3-((5-(1-(2,2-difluoroethyl)-2-methyl-1H-imidazo [4,5-b] pyrazin-6-yl)-7H-pyrrolo [2,3-d]pyrimidin-2-yl)amino)-1-methylcyclobutyl)pyrrolidin-2-one [2826];
1-((1 s,3 s)-3-((5-(1-(2,2-difluoroethyl)-2-methyl-1H-imidazo [4,5-b]pyrazin-6-yl)-7H-pyrrolo [2,3-d]pyrimidin-2-yl)amino)-1-methylcyclobutyl)pyrrolidin-2-one [2827];
(1s,3s)-3-((5-(1-(2,2-difluoroethyl)-2-methyl-1H-imidazo [4,5-b] pyrazin-6-yl)-7H-pyrrolo [2,3-]pyrimidin-2-yl)amino)-1-methylcyclobutan-1-ol [2828];
(1r,3r)-3-((5-(1-(2,2-difluoroethyl)-2-methyl-1H-imidazo [4,5-b] pyrazin-6-yl)-7H-pyrrolo [2,3-d]pyrimidin-2-yl)amino)-1-methylcyclobutan-1-ol [2829];
(1s,4s)-4-((5-(1-(2,2-difluoroethyl)-2-methyl-1H-imidazo [4,5-b] pyrazin-6-yl)-7H-pyrrolo [2,3-d]pyrimidin-2-yl)amino)-N,N-dimethylcyclohexane-1-carboxamide [2830];
((1s,4s)-4-((5-(1-(2,2-difluoroethyl)-2-methyl-1H-imidazo[4,5-b]pyrazin-6-yl)-7H-pyrrolo [2,3-d]pyrimidin-2-yl)amino)cyclohexyl)(pyrrolidin-1-yl)methanone [2831];
N-((1s,4s)-4-((5-(1-(2,2-difluoroethyl)-2-methyl-1H-imidazo [4,5-b]pyrazin-6-yl)-7H-pyrrolo [2,3-d]pyrimidin-2-yl)amino)cyclohexypacetamide [2832];
N-((1r,4r)-4-((5-(1-(2,2-difluoroethyl)-2-methyl-1H-imidazo [4,5-b]pyrazin-6-yl)-7H-pyrrolo [2,3-d]pyrimidin-2-yl)amino)cyclohexypacetamide [2833];
1-((1s,4s)-4-((5-(1-(2,2-difluoroethyl)-2-methyl-1H-imidazo [4,5-b]pyrazin-6-yl)-7H-pyrrolo [2,3-d]pyrimidin-2-yl)amino)cyclohexyl)pyrrolidin-2-one [2834];
5-(1-(2,2-difluoroethyl)-2-methyl-1H-imidazo[4,5-b]pyrazin-6-yl)-N-((1s,4s)-4-methoxycyclohexyl)-7H-pyrrolo [2,3-d]pyrimidin-2-amine [2835];
2-(((1s,4s)-4-((5-(1-(2,2-difluoroethyl)-2-methyl-1H-imidazo [4,5-b]pyrazin-6-yl)-7H-pyrrolo [2,3-d]pyrimidin-2-yl)amino)cyclohexyl)oxy)ethan-1-ol [2836];
2-(((1r,4r)-4-((5-(1-(2,2-difluoroethyl)-2-methyl-1H-imidazo [4,5-b]pyrazin-6-yl)-7H-pyrrolo [2,3-d]pyrimidin-2-yl)amino)cyclohexyl)oxy)ethan-1-ol [2837];
N-((1r,4r)-4-((5-(1-(2,2-difluoroethyl)-2-methyl-1H-imidazo [4,5-b]pyrazin-6-yl)-7H-pyrrolo [2,3-d]pyrimidin-2-yl)amino)-1-methylcyclohexyl)acetamide [2838];
(1s,4s)-4-((5-(1-(2,2-difluoroethyl)-2-methyl-1H-imidazo [4,5-b] pyrazin-6-yl)-7H-pyrrolo [2,3-d]pyrimidin-2-yl)amino)-1-methylcyclohexan-1-ol [2839];
(1r,4r)-4-((5-(1-(2,2-difluoroethyl)-2-methyl-1H-imidazo [4,5-b] pyrazin-6-yl)-7H-pyrrolo [2,3-d]pyrimidin-2-yl)amino)-1-methylcyclohexan-1-ol [2840];
(1s,4s)-4-((5-(1-(2,2-difluoroethyl)-2-methyl-1H-imidazo [4,5-b] pyrazin-6-yl)-7H-pyrrolo [2,3-d]pyrimidin-2-yl)amino)-1-ethylcyclohexan-1-ol [2841];
1-(4-((5-(1-(2,2-difluoroethyl)-2-methyl-1H-imidazo [4,5-b] pyrazin-6-yl)-7H-pyrrolo [2,3-d]pyrimidin-2-yl)amino)piperidin-1-yl)ethan-1-one [2842];
(S)-5-((5-(1-(2,2-difluoroethyl)-2-methyl-1H-imidazo [4,5-b]pyrazin-6-yl)-7H-pyrrolo [2,3-d]pyrimidin-2-yl)amino)-1-methylpiperidin-2-one [2843];
(R)-5-((5-(1-(2,2-difluoroethyl)-2-methyl-1H-imidazo [4,5-b]pyrazin-6-yl)-7H-pyrrolo [2,3-d]pyrimidin-2-yl)amino)-1-methylpiperidin-2-one [2844];
1-(2-((5-(1-(2,2-difluoroethyl)-2-methyl-1H-imidazo [4,5-b] pyrazin-6-yl)-7H-pyrrolo [2,3-d]pyrimidin-2-yl)amino)-7-azaspiro [3 . 5 ]nonan-7-yl)ethan-1-one [2845];
1-((3 aR,5 s,6aS)-5-((5-(1-(2,2-difluoroethyl)-2-methyl-1H-imidazo [4,5-b] pyrazin-6-yl)-7H-pyrrolo [2,3-d]pyrimidin-2-yl)amino)hexahydrocyclopenta[c]pyrrol-2(1H)-yl)ethan-1-one [2846];
1-((3 aR,5 r,6aS)-5-((5-(1-(2,2-difluoroethyl)-2-methyl-1H-imidazo [4,5-b]pyrazin-6-yl)-7H-pyrrolo [2,3-d]pyrimidin-2-yl)amino)hexahydrocyclopenta[c]pyrrol-2(1H)-yl)ethan-1-one [2847];
1-(7-((5-(1-(2,2-difluoroethyl)-2-methyl-1H-imidazo [4,5-b] pyrazin-6-yl)-7H-pyrrolo [2,3-d]pyrimidin-2-yl)amino)-2-azaspiro [3 . 5 ]nonan-2-yl)ethan-1-one [2848];
5-(1-(2,2-difluoroethyl)-2-methyl-1H-imidazo [4,5-b] pyrazin-6-yl)-N-(2-oxaspiro [3 .5 ]nonan-7-yl)-7H-pyrrolo [2,3-d] pyrimidin-2-amine [2849];
5-(1-(2,2-difluoroethyl)-2-methyl-1H-imidazo [4,5-b] pyrazin-6-yl)-N-(1,4-dioxaspiro [4 .5]decan-8-yl)-7H-pyrrolo [2,3-d] pyrimidin-2-amine [2850];
(1 s,4s)-4-((5-(1-(2,2-difluoroethyl)-2-methyl-1H-imidazo [4,5-b] pyrazin-6-yl)-4-(methylamino)-7H-pyrrolo [2,3-d]pyrimidin-2-yl)amino)-N,N-dimethylcyclohexane-1-carboxamide [2851];
(1 r,4r)-4-((5-(1-(2,2-difluoroethyl)-2-methyl-1H-imidazo [4,5-1)] pyrazin-6-yl)-4-(methylamino)-7H-pyrrolo [2,3-d]pyrimidin-2-yl)amino)-N,N-dimethylcyclohexane-1-carboxamide [2852];
((1 s,4s)-4-((5-(1-(2,2-difluoroethyl)-2-methyl-1H-imidazo [4,5-b]pyrazin-6-yl)-4-(methylamino)-7H-pyrrolo [2,3-d]pyrimidin-2-yl)amino)cyclohexyl)(pyrrolidin-1-yl)methanone [2853];
((1 r,4r)-4-((5-(1-(2,2-difluoroethyl)-2-methyl-1H-imidazo [4,5-b] pyrazin-6-yl)-4-(methylamino)-7H-pyrrolo [2,3-d]pyrimidin-2-yl)amino)cyclohexyl)(pyrrolidin-1-yl)methanone [2854];
N-((1 s,4s)-4-((5-(1-(2,2-difluoroethyl)-2-methyl-1H-imidazo [4,5-b]pyrazin-6-yl)-4-(methylamino)-7H-pyrrolo [2,3-d]pyrimidin-2-yl)amino)cyclohexypacetamide [2855];
N-((1 r,4r)-4-((5-(1-(2,2-difluoroethyl)-2-methyl-1H-imidazo [4,5-b]pyrazin-6-yl)-4-(methylamino)-7H-pyrrolo [2,3-d]pyrimidin-2-yl)amino)cyclohexypacetamide [2856];
1-((1 s,4s)-4-((5-(1-(2,2-difluoroethyl)-2-methyl-1H-imidazo [4,5-b]pyrazin-6-yl)-4-(methylamino)-7H-pyrrolo [2,3-d]pyrimidin-2-yl)amino)cyclohexyl)pyrrolidin-2-one [2857];
1-((1 r,4r)-4-((5-(1-(2,2-difluoroethyl)-2-methyl-1H-imidazo [4,5-b] pyrazin-6-yl)-4-(methylamino)-7H-pyrrolo [2,3-d]pyrimidin-2-yl)amino)cyclohexyl)pyrrolidin-2-one [2858];
1-((1 s,4s)-4-((5-(1-(2,2-difluoroethyl)-2-methyl-1H-imidazo [4,5-b]pyrazin-6-yl)-4-(methylamino)-7H-pyrrolo [2,3-d]pyrimidin-2-yl)amino)-1-methylcyclohexyl)pyrrolidin-2-one
1-((1r,4r)-4-((5-(1-(2,2-difluoroethyl)-2-methyl-1H-imidazo[4,5-b]pyrazin-6-yl)-4-(methylamino)-7H-pyrrolo[2,3-d]pyrimidin-2-yl)amino)-1-methylcyclohexyl)pyrrolidin-2-one [2860];
5-(1-(2,2-difluoroethyl)-2-methyl-1H-imidazo[4,5-b]pyrazin-6-yl)-N²-((1s,4s)-4-methoxycyclohexyl)-N⁴-methyl-7H-pyrrolo[2,3-d]pyrimidine-2,4-diamine [2861];
5-(1-(2,2-difluoroethyl)-2-methyl-1H-imidazo[4,5-b]pyrazin-6-yl)-N⁴-methyl-N²-(2-oxaspiro[3.5]nonan-7-yl)-7H-pyrrolo[2,3-d]pyrimidine-2,4-diamine [2862];
(1s,3s)-3-((5-([1,2,4]triazolo[4,3-a]pyridin-6-yl)-7H-pyrrolo[2,3-d]pyrimidin-2-yl)amino)-N,N,1-trimethylcyclobutane-1-carboxamide [2863];
(1r,3r)-3-((5-([1,2,4]triazolo[4,3-a]pyridin-6-yl)-7H-pyrrolo[2,3-d]pyrimidin-2-yl)amino)-N,N,1-trimethylcyclobutane-1-carboxamide [2864];
((1r,3r)-3-((5-([1,2,4]triazolo[4,3-a]pyridin-6-yl)-7H-pyrrolo[2,3-d]pyrimidin-2-yl)amino)-1-methylcyclobutyl)(pyrrolidin-1-yl)methanone [2865];
((1s,3s)-3-((5-([1,2,4]triazolo[4,3-a]pyridin-6-yl)-7H-pyrrolo[2,3-d]pyrimidin-2-yl)amino)-1-methylcyclobutyl)(pyrrolidin-1-yl)methanone [2866];
N-((1s,3s)-3-((5-([1,2,4]triazolo[4,3-a]pyridin-6-yl)-7H-pyrrolo[2,3-d]pyrimidin-2-yl)amino)-1-methylcyclobutypacetamide [2867];
N-((1r,3r)-3-((5-([1,2,4]triazolo[4,3-a]pyridin-6-yl)-7H-pyrrolo[2,3-d]pyrimidin-2-yl)amino)-1-methylcyclobutypacetamide [2868];
N-((1s,3s)-3-((5-([1,2,4]triazolo[4,3-a]pyridin-6-yl)-7H-pyrrolo[2,3-d]pyrimidin-2-yl)amino)-1-methylcyclobutyl)propionamide [2869];
N-((1r,3r)-3-((5-([1,2,4]triazolo[4,3-a]pyridin-6-yl)-7H-pyrrolo[2,3-d]pyrimidin-2-yl)amino)-1-methylcyclobutyl)propionamide P8701;
1-((1r,3r)-3-((5-([1,2,4]triazolo[4,3-a]pyridin-6-yl)-7H-pyrrolo[2,3-d]pyrimidin-2-yl)amino)-1-methylcyclobutyl)pyrrolidin-2-one P8711;
1-((1s,3s)-3-((5-([1,2,4]triazolo[4,3-a]pyridin-6-yl)-7H-pyrrolo[2,3-d]pyrimidin-2-yl)amino)-1-methylcyclobutyl)pyrrolidin-2-one P8721;
(1s,3s)-3-((5-([1,2,4]triazolo[4,3-a]pyridin-6-yl)-7H-pyrrolo[2,3-d]pyrimidin-2-yl)amino)-1-methylcyclobutan-1-ol [2873];
(1r,3r)-3-((5-([1,2,4]triazolo[4,3-a]pyridin-6-yl)-7H-pyrrolo[2,3-d]pyrimidin-2-yl)amino)-1-methylcyclobutan-1-ol [2874];
(1s,4s)-4-((5-([1,2,4]triazolo[4,3-a]pyridin-6-yl)-7H-pyrrolo[2,3-d]pyrimidin-2-yl)amino)-N,N-dimethylcyclohexane-1-carboxamide P8751;
((1s,4s)-4-((5-([1,2,4]triazolo[4,3-a]pyridin-6-yl)-7H-pyrrolo[2,3-d]pyrimidin-2-yl)amino)cyclohexyl)(pyrrolidin-1-yl)methanone [2876];
N-((1s,4s)-4-((5-([1,2,4]triazolo[4,3-a]pyridin-6-yl)-7H-pyrrolo[2,3-d]pyrimidin-2-yl)amino)cyclohexypacetamide [2877];
N-((1r,4r)-4-((5-([1,2,4]triazolo[4,3-a]pyridin-6-yl)-7H-pyrrolo[2,3-d]pyrimidin-2-yl)amino)cyclohexypacetamide [2878];
1-((1s,4s)-4-((5-([1,2,4]triazolo[4,3-a]pyridin-6-yl)-7H-pyrrolo[2,3-d]pyrimidin-2-yl)amino)cyclohexyl)pyrrolidin-2-one [2879];
5-([1,2,4]triazolo [4,3-a]pyridin-6-yl)-N-((1s,4s)-4-methoxycyclohexyl)-7H-pyrrolo [2,3-d]pyrimidin-2-amine [2880];
2-(((1s,4s)-4-45-([1,2,4]triazolo[4,3-a]pyridin-6-yl)-7H-pyrrolo[2,3-d]pyrimidin-2-yl)amino)cyclohexyl)oxy)ethan-1-ol [2881];
2-(((1r,4r)-4-((5-([1,2,4]triazolo[4,3-a]pyridin-6-yl)-7H-pyrrolo[2,3-d]pyrimidin-2-yl)amino)cyclohexyl)oxy)ethan-1-ol [2882];
N-((1r,40-4-45-([1,2,4]triazolo [4,3-a]pyridin-6-yl)-7H-pyrrolo [2,3-d]pyrimidin-2-yl)amino)-1-methylcyclohexyl)acetamide [2883];
(1r,4r)-4-((5-([1,2,4]triazolo[4,3-a]pyridin-6-yl)-7H-pyrrolo[2,3-d]pyrimidin-2-yl)amino)-1-methylcyclohexan-1-ol [2884];
(1s,4s)-4-((5-([1,2,4]triazolo[4,3-a]pyridin-6-yl)-7H-pyrrolo[2,3-d]pyrimidin-2-yl)amino)-1-methylcyclohexan-1-ol [2885];
(1s,4s)-4-((5-([1,2,4]triazolo[4,3-a]pyridin-6-yl)-7H-pyrrolo[2,3-d]pyrimidin-2-yl)amino)-1-ethylcyclohexan-1-ol [2886];
1-(4-((5-([1,2,4]triazolo[4,3-a]pyridin-6-yl)-7H-pyrrolo[2,3-d]pyrimidin-2-yl)amino)piperidin-1-yl)ethan-1-one [2887];
(R)-5-45-([1,2,4]triazolo[4,3-a]pyridin-6-yl)-7H-pyrrolo[2,3-d]pyrimidin-2-yl)amino)-1-methylpiperidin-2-one [2888];
(S)-5-((5-([1,2,4]triazolo[4,3-a]pyridin-6-yl)-7H-pyrrolo[2,3-d]pyrimidin-2-yl)amino)-1-methylpiperidin-2-one [2889];
1-(2-45-([1,2,4]triazolo[4,3-a]pyridin-6-yl)-7H-pyrrolo[2,3-d]pyrimidin-2-yl)amino)-7-azaspiro[3.5]nonan-7-yl)ethan-1-one [2890];
1-((3aR,5s,6aS)-5-45-([1,2,4]triazolo[4,3-a]pyridin-6-yl)-7H-pyrrolo[2,3-d]pyrimidin-2-yl)amino)hexahydrocyclopenta[c]pyrrol-2(1H)-yl)ethan-1-one [2891];
1-((3aR,5r,6aS)-5-((5-([1,2,4]triazolo[4,3-a]pyridin-6-yl)-7H-pyrrolo[2,3-d]pyrimidin-2-yl)amino)hexahydrocyclopenta[c]pyrrol-2(1H)-yl)ethan-1-one [2892];
1-(7-45-([1,2,4]triazolo[4,3-a]pyridin-6-yl)-7H-pyrrolo[2,3-d]pyrimidin-2-yl)amino)-2-azaspiro[3.5]nonan-2-yl)ethan-1-one [2893];
5-([1,2,4]triazolo[4,3-a]pyridin-6-yl)-N-(2-oxaspiro[3.5]nonan-7-yl)-7H-pyrrolo[2,3-d]pyrimidin-2-amine [2894];
5-([1,2,4]triazolo[4,3-a]pyridin-6-yl)-N-(1,4-dioxaspiro[4.5]decan-8-yl)-7H-pyrrolo[2,3-d]pyrimidin-2-amine [2895];
(1s,3s)-3-((5-([1,2,4]triazolo[4,3-a]pyridin-6-yl)-4-methoxy-7H-pyrrolo[2,3-d]pyrimidin-2-yl)amino)-N,N,1-trimethylcyclobutane-1-carboxamide [2896];
(1r,3r)-3-((5-([1,2,4]triazolo[4,3-a]pyridin-6-yl)-4-methoxy-7H-pyrrolo[2,3-d]pyrimidin-2-yl)amino)-N,N,1-trimethylcyclobutane-1-carboxamide [2897];
((1s,3s)-3-((5-([1,2,4]triazolo[4,3-a]pyridin-6-yl)-4-methoxy-7H-pyrrolo[2,3-d]pyrimidin-2-yl)amino)-1-methylcyclobutyl)(pyrrolidin-1-yl)methanone [2898];
((1r,3r)-3-((5-([1,2,4]triazolo[4,3-a]pyridin-6-yl)-4-methoxy-7H-pyrrolo[2,3-d]pyrimidin-2-yl)amino)-1-methylcyclobutyl)(pyrrolidin-1-yl)methanone [2899];
N-((1s,3s)-3-((5-([1,2,4]triazolo[4,3-a]pyridin-6-yl)-4-methoxy-7H-pyrrolo[2,3-d]pyrimidin-2-yl)amino)-1-methylcyclobutypacetamide [2900];
N-((1r,3r)-3-((5-([1,2,4]triazolo[4,3-a]pyridin-6-yl)-4-methoxy-7H-pyrrolo[2,3-d]pyrimidin-2-yl)amino)-1-methylcyclobutypacetamide [2901];
N-((1s,3s)-3-((5-([1,2,4]triazolo[4,3-a]pyridin-6-yl)-4-methoxy-7H-pyrrolo[2,3-d]pyrimidin-2-yl)amino)-1-methylcyclobutyl)propionamide [2902];
N-((1r,3r)-3-((5-([1,2,4]triazolo[4,3-a]pyridin-6-yl)-4-methoxy-7H-pyrrolo[2,3-d]pyrimidin-2-yl)amino)-1-methylcyclobutyl)propionamide [2903];
1-((1r,3r)-3-((5-([1,2,4]triazolo[4,3-a]pyridin-6-yl)-4-methoxy-7H-pyrrolo[2,3-d]pyrimidin-2-yl)amino)-1-methylcyclobutyppyrrolidin-2-one [2904];
1-((1s,3s)-3-((5-([1,2,4]triazolo[4,3-a]pyridin-6-yl)-4-methoxy-7H-pyrrolo[2,3-d]pyrimidin-2-yl)amino)-1-methylcyclobutyppyrrolidin-2-one [2905];
(1s,3s)-3-((5-([1,2,4]triazolo[4,3-a]pyridin-6-yl)-4-methoxy-7H-pyrrolo[2,3-d]pyrimidin-2-yl)amino)-1-methylcyclobutan-1-ol [2906];
(1r,3r)-3-((5-([1,2,4]triazolo[4,3-a]pyridin-6-yl)-4-methoxy-7H-pyrrolo[2,3-d]pyrimidin-2-yl)amino)-1-methylcyclobutan-1-ol [2907];
(1s,4s)-4-((5-([1,2,4]triazolo[4,3-a]pyridin-6-yl)-4-methoxy-7H-pyrrolo[2,3-d]pyrimidin-2-yl)amino)-N,N-dimethylcyclohexane-1-carboxamide [2908];
((1s,4s)-4-((5-([1,2,4]triazolo[4,3-a]pyridin-6-yl)-4-methoxy-7H-pyrrolo[2,3-d]pyrimidin-2-yl)amino)cyclohexyl)(pyrrolidin-1-yl)methanone [2909];
N-((1s,4s)-4-((5-([1,2,4]triazolo[4,3-a]pyridin-6-yl)-4-methoxy-7H-pyrrolo[2,3-d]pyrimidin-2-yl)amino)cyclohexypacetamide [2910];
N-((1r,4r)-4-((5-([1,2,4]triazolo[4,3-a]pyridin-6-yl)-4-methoxy-7H-pyrrolo[2,3-d]pyrimidin-2-yl)amino)cyclohexypacetamide [2911];
1-((1s,4s)-4-((5-([1,2,4]triazolo[4,3-a]pyridin-6-yl)-4-methoxy-7H-pyrrolo[2,3-d]pyrimidin-2-yl)amino)cyclohexyl)pyrrolidin-2-one [2912];
5-([1,2,4]triazolo[4,3-a]pyridin-6-yl)-4-(methoxy-d₃)-N-((1s,4s)-4-methoxycyclohexyl)-7H-pyrrolo[2,3-d]pyrimidin-2-amine [2913];
2-(((1s,4s)-4-((5-([1,2,4]triazolo[4,3-a]pyridin-6-yl)-4-methoxy-7H-pyrrolo[2,3-d]pyrimidin-2-yl)amino)cyclohexyl)oxy)ethan-1-ol [2914];
2-(((1r,4r)-4-((5-([1,2,4]triazolo[4,3-a]pyridin-6-yl)-4-methoxy-7H-pyrrolo[2,3-d]pyrimidin-2-yl)amino)cyclohexyl)oxy)ethan-1-ol [2915];
N-((1r,4r)-4-((5-([1,2,4]triazolo[4,3-a]pyridin-6-yl)-4-methoxy-7H-pyrrolo[2,3-d]pyrimidin-2-yl)amino)-1-methylcyclohexyl)acetamide [2916];
(1r,4r)-4-((5-([1,2,4]triazolo[4,3-a]pyridin-6-yl)-4-methoxy-7H-pyrrolo[2,3-d]pyrimidin-2-yl)amino)-1-methylcyclohexan-1-ol [2917];
(1s,4s)-4-((5-([1,2,4]triazolo[4,3-a]pyridin-6-yl)-4-methoxy-7H-pyrrolo[2,3-d]pyrimidin-2-yl)amino)-1-methylcyclohexan-1-ol [2918];
(1s,4s)-4-((5-([1,2,4]triazolo[4,3-a]pyridin-6-yl)-4-methoxy-7H-pyrrolo[2,3-d]pyrimidin-2-yl)amino)-1-ethylcyclohexan-1-ol [2919];
(1r,4r)-4-((5-([1,2,4]triazolo[4,3-a]pyridin-6-yl)-4-(methoxy-d₃)-7H-pyrrolo[2,3-d]pyrimidin-2-yl)amino)-1-methylcyclohexan-1-ol [2920];
(1s,4s)-4-((5-([1,2,4]triazolo[4,3-a]pyridin-6-yl)-4-(methoxy-d₃)-7H-pyrrolo[2,3-d]pyrimidin-2-yl)amino)-1-methylcyclohexan-1-ol [2921];
1-(4-((5-([1,2,4]triazolo[4,3-a]pyridin-6-yl)-4-methoxy-7H-pyrrolo[2,3-d]pyrimidin-2-yl)amino)piperidin-1-yl)ethan-1-one [2922];
(S)-5-((5-([1,2,4]triazolo[4,3-a]pyridin-6-yl)-4-methoxy-7H-pyrrolo[2,3-d]pyrimidin-2-yl)amino)-1-methylpiperidin-2-one [2923];
(R)-5-((5-([1,2,4]triazolo[4,3-a]pyridin-6-yl)-4-methoxy-7H-pyrrolo[2,3-d]pyrimidin-2-yl)amino)-1-methylpiperidin-2-one [2924];
1-(2-((5-([1,2,4]triazolo[4,3-a]pyridin-6-yl)-4-methoxy-7H-pyrrolo[2,3-d]pyrimidin-2-yl)amino)-7-azaspiro[3.5]nonan-7-yl)ethan-1-one [2925];
1-((3aR,5s,6aS)-5-45-([1,2,4]triazolo[4,3-a]pyridin-6-yl)-4-methoxy-7H-pyrrolo[2,3-d]pyrimidin-2-yl)amino)hexahydrocyclopenta[c]pyrrol-2(1H)-yl)ethan-1-one [2926];
1-((3aR,5r,6aS)-5-((5-([1,2,4]triazolo [4,3-a]pyridin-6-yl)-4-methoxy-7H-pyrrolo [2,3-d]pyrimidin-2-yl)amino)hexahydrocyclopenta [c]pyrrol-2(1H)-yl)ethan-1-one [2927];
1-(7-((5-([1,2,4]triazolo [4,3-a]pyridin-6-yl)-4-methoxy-7H-pyrrolo [2,3-d]pyrimidin-2-yl)amino)-2-azaspiro [3 .5] nonan-2-yl)ethan-1-one [2928];
5-([1,2,4]triazolo [4,3-a]pyridin-6-yl)-4-methoxy-N-(2-oxaspiro [3 .5]nonan-7-yl)-7H-pyrrolo [2,3-d]pyrimidin-2-amine [2929];
5-([1,2,4]triazolo [4,3-a]pyridin-6-yl)-4-methoxy-N-(1,4-dioxaspiro [4.5] de can-8-yl)-7H-pyrrolo [2,3-d]pyrimidin-2-amine [2930];
(1s,3s)-N,N,1-trimethyl-3-((5-(3-methyl-[1,2,4]triazolo[4,3-a]pyridin-6-yl)-7H-pyrrolo [2,3-d]pyrimidin-2-yl)amino)cyclobutane-1-carboxamide [2931];
(1r,30-N,N,1-trimethyl-3-((5-(3-methyl-[1,2,4]triazolo[4,3-a]pyridin-6-yl)-7H-pyrrolo [2,3-d]pyrimidin-2-yl)amino)cyclobutane-1-carboxamide [2932];
((1s,3s)-1-methyl-3-((5-(3-methyl-[1,2,4]triazolo [4,3-a]pyridin-6-yl)-7H-pyrrolo [2,3-d]pyrimidin-2-yl)amino)cyclobutyl)(pyrrolidin-1-yl)me thanone [2933];
((1r,3r)-1-methyl-3-((5-(3-methyl-[1,2,4]triazolo [4,3-a]pyridin-6-yl)-7H-pyrrolo [2,3-d] pyrimidin-2-yl)amino)cyclobutyl)(pyrrolidin-1-yl)methanone [2934];
N-((1s,3s)-1-methyl-3-((5-(3-methyl-[1,2,4]triazolo [4,3-a]pyridin-6-yl)-7H-pyrrolo [2,3-d]pyrimidin-2-yl)amino)cyclobutypacetamide [2935];
N-((1r,3r)-1-methyl-3-((5-(3-methyl-[1,2,4]triazolo [4,3-a]pyridin-6-yl)-7H-pyrrolo [2,3-d]pyrimidin-2-yl)amino)cyclobutypacetamide [2936];
N-((1s,3s)-1-methyl-3-((5-(3-methyl-[1,2,4]triazolo [4,3-a]pyridin-6-yl)-7H-pyrrolo [2,3-d]pyrimidin-2-yl)amino)cyclobutyl)propionamide [2937];
N-((1r,3r)-1-methyl-3-((5-(3-methyl-[1,2,4]triazolo [4,3-a]pyridin-6-yl)-7H-pyrrolo [2,3-d]pyrimidin-2-yl)amino)cyclobutyl)propionamide [2938];
1-((1r,3r)-1-methyl-3-((5-(3-methyl-[1,2,4]triazolo [4,3-a] pyridin-6-yl)-7H-pyrrolo [2,3-d]pyrimidin-2-yl)amino)cyclobutyppyrrolidin-2-one [2939];
1-((1s,3s)-1-methyl-3-45-(3-methyl41,2,41triazolo [4,3-a] pyridin-6-yl)-7H-pyrrolo [2,3-d]pyrimidin-2-yl)amino)cyclobutyppyrrolidin-2-one [2940];
(1s,3s)-1-methyl-3-((5-(3-methyl-[1,2,4]triazolo [4,3-a]pyridin-6-yl)-7H-pyrrolo [2,3-d] pyrimidin-2-yl)amino)cyclobutan-1-ol [2941];
(1r,3r)-1-methyl-3-((5-(3-methyl-[1,2,4]triazolo [4,3-a]pyridin-6-yl)-7H-pyrrolo [2,3-d]pyrimidin-2-yl)amino)cyclobutan-1-ol [2942];
(1s,4s)-N,N-dimethyl-4-((5-(3-methyl-[1,2,4]triazolo [4,3-a] pyridin-6-yl)-7H-pyrrolo [2,3-d]pyrimidin-2-yl)amino)cyclohexane-1-carboxamide [2943];
((1s,4s)-4-((5-(3-methyl-[1,2,4]triazolo [4,3-a]pyridin-6-yl)-7H-pyrrolo [2,3-cl]pyrimidin-2-yl)amino)cyclohexyl)(pyrrolidin-1-yl)methanone [2944];
N-((1s,4s)-4-((5-(3-methyl-[1,2,4]triazolo[4,3-a]pyridin-6-yl)-7H-pyrrolo [2,3-cl]pyrimidin-2-yl)amino)cyclohexypacetamide [2945];
N-((1r,4r)-4-((5-(3-methyl-[1,2,4]triazolo[4,3-a]pyridin-6-yl)-7H-pyrrolo [2,3-cl]pyrimidin-2-yl)amino)cyclohexypacetamide [2946];
1-((1s,4s)-4-((5-(3-methyl-[1,2,4]triazolo[4,3-a]pyridin-6-yl)-7H-pyrrolo [2,3-cl]pyrimidin-2-yl)amino)cyclohexyl)pyrrolidin-2-one [2947];
2-(((1s,4s)-4-45-(3-methyl-[1,2,4]triazolo [4,3-a]pyridin-6-yl)-7H-pyrrolo [2,3-cl]pyrimidin-2-yl)amino)cyclohexyl)oxy)ethan-1-ol [2948];
2-(((1r,4r)-4-((5-(3-methyl-[1,2,4]triazolo[4,3-a]pyridin-6-yl)-7H-pyrrolo [2,3-cl]pyrimidin-2-yl)amino)cyclohexyl)oxy)ethan-1-ol [2949];
N-((1r,4r)-1-methyl-4-((5-(3-methyl-[1,2,4]triazolo [4,3-a]pyridin-6-yl)-7H-pyrrolo [2,3-cl]pyrimidin-2-yl)amino)cyclohexypacetamide [2950];
(1r,4r)-1-methyl-4-((5-(3-methyl-[1,2,4]triazolo [4,3-a]pyridin-6-yl)-7H-pyrrolo [2,3-cl]pyrimidin-2-yl)amino)cyclohexan-1-ol [2951];
(1s,4s)-1-methyl-4-((5-(3-methyl-[1,2,4]triazolo [4,3-a]pyridin-6-yl)-7H-pyrrolo [2,3-cl]pyrimidin-2-yl)amino)cyclohexan-1-ol [2952];
(1s,4s)-1-ethyl-4-((5-(3-methyl-[1,2,4]triazolo [4,3-a]pyridin-6-yl)-7H-pyrrolo [2,3-cl]pyrimidin-2-yl)amino)cyclohexan-1-ol [2953];
1-(4-((5-(3-methyl-[1,2,4]triazolo [4,3-a]pyridin-6-yl)-7H-pyrrolo [2,3-cl]pyrimidin-2-yl)amino)piperidin-1-yl)ethan-1-one [2954];
(S)-1-methyl-5-((5-(3-methyl-[1,2,4]triazolo [4,3-a]pyridin-6-yl)-7H-pyrrolo [2,3-cl]pyrimidin-2-yl)amino)piperidin-2-one [2955];
(R)-1-methyl-5-((5-(3-methyl-[1,2,4]triazolo [4,3-a]pyridin-6-yl)-7H-pyrrolo [2,3-cl]pyrimidin-2-yl)amino)piperidin-2-one [2956];
1-(2-((5-(3-methyl-[1,2,4]triazolo [4,3-a]pyridin-6-yl)-7H-pyrrolo [2,3-cl]pyrimidin-2-yl)amino)-7-azaspiro [3 .5]nonan-7-yl)ethan-1-one [2957];
1-((3aR,5s,6aS)-5-45-(3-methyl-[1,2,4]triazolo [4,3-a]pyridin-6-yl)-7H-pyrrolo [2,3-cl]pyrimidin-2-yl)amino)hexahydrocyclopenta[c]pyrrol-2(1H)-yl)ethan-1-one [2958];
1-((3aR,5r,6aS)-5-((5-(3-methyl-[1,2,4]triazolo [4,3-a]pyridin-6-yl)-7H-pyrrolo [2,3-cl]pyrimidin-2-yl)amino)hexahydrocyclopenta[c]pyrrol-2(1H)-yl)ethan-1-one [2959];
1-(7-((5-(3-methyl-[1,2,4]triazolo [4,3-a]pyridin-6-yl)-7H-pyrrolo [2,3-cl]pyrimidin-2-yl)amino)-2-azaspiro [3 .5]nonan-2-yl)ethan-1-one [2960];
5-(3-methyl-[1,2,4]triazolo [4,3-a]pyridin-6-yl)-N-(2-oxaspiro [3 .5]nonan-7-yl)-7H-pyrrolo [2,3-d]pyrimidin-2-amine [2961];
5-(3-methyl-[1,2,4]triazolo [4,3-a]pyridin-6-yl)-N-(1,4-dioxaspiro [4 .5] decan-8-yl)-7H-pyrrolo [2,3-d]pyrimidin-2-amine [2962];
(1r,3r)-3-((4-methoxy-5-(3-methyl-[1,2,4]triazolo [4,3-a]pyridin-6-yl)-7H-pyrrolo [2,3-d]pyrimidin-2-yl)amino)-N,N, 1-trimethylcyclobutane-1-carboxamide [2963];
(1 s,3 s)-3-((4-methoxy-5-(3-methyl-[1,2,4]triazolo [4,3-a]pyridin-6-yl)-7H-pyrrolo [2,3-d]pyrimidin-2-yl)amino)-N,N, 1-trimethylcyclobutane-1-carboxamide [2964];
((1r,3r)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)(pyrrolidin-1-yl)methanone [2965];
((1 s,3 s)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)(pyrrolidin-1-yl)methanone [2966];
N-((1 s,3 s)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)acetamide [2967];
N-((1r,3r)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)acetamide [2968];
N-((1 s,3 s)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)propionamide [2969];
N-((1r,3r)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)propionamide [2970];
1-((1r,3r)-3-((4-methoxy-5-(3-methyl-[1,2,4]triazolo [4,3-a] pyridin-6-yl)-7H-pyrrolo [2,3-d]pyrimidin-2-yl)amino)-1-methylcyclobutyl)pyrrolidin-2-one [2971];
1-((1 s,3 s)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutyl)pyrrolidin-2-one [2972];
(1 s,3 s)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutan-1-ol [2973];
(1r,3r)-3-((4-methoxy-5-(3-methyl-[1,2,4]triazolo [4,3-a]pyridin-6-yl)-7H-pyrrolo [2,3-d]pyrimidin-2-yl)amino)-1-methylcyclobutan-1-ol [2974];
(1 s,4 s)-4-((4-methoxy-5-(3-methyl-[1,2,4]triazolo [4,3-a]pyridin-6-yl)-7H-pyrrolo [2,3-d]pyrimidin-2-yl)amino)-N,N-dimethylcyclohexane-1-carboxamide [2975];
((1 s,4 s)-4-((4-methoxy-5-(3-methyl-[1,2,4]triazolo [4,3-a]pyridin-6-yl)-7H-pyrrolo [2,3-d]pyrimidin-2-yl)amino)cyclohexyl)(pyrrolidin-1-yl)methanone [2976];
N-((1 s,4 s)-4-((4-methoxy-5-(3-methyl-[1,2,4]triazolo [4,3-a]pyridin-6-yl)-7H-pyrrolo [2,3-d]pyrimidin-2-yl)amino)cyclohexypacetamide [2977];
N-((1r,4r)-4-((4-methoxy-5-(3-methyl-[1,2,4]triazolo [4,3-a]pyridin-6-yl)-7H-pyrrolo [2,3-d]pyrimidin-2-yl)amino)cyclohexypacetamide [2978];
1-((1s,4s)-4-((4-methoxy-5-(3-methyl-[1,2,4]triazolo [4,3-a]pyridin-6-yl)-7H-pyrrolo [2,3-d]pyrimidin-2-yl)amino)cyclohexyl)pyrrolidin-2-one [2979];
5-([1,2,4]triazolo [4,3-a]pyridin-6-yl)-4-methoxy-N-((1s,4s)-4-methoxycyclohexyl)-7H-pyrrolo [2,3-d]pyrimidin-2-amine [2980];
2-(((1s,4s)-4-((4-methoxy-5-(3-methyl-[1,2,4]triazolo[4,3-a]pyridin-6-yl)-7H-pyrrolo [2,3-d]pyrimidin-2-yl)amino)cyclohexyl)oxy)ethan-1-ol [2981];
2-(((1r,4r)-4-((4-methoxy-5-(3-methyl-[1,2,4]triazolo [4,3-a]pyridin-6-yl)-7H-pyrrolo [2,3-d]pyrimidin-2-yl)amino)cyclohexyl)oxy)ethan-1-ol [2982];
N-((1r,4r)-4-((4-methoxy-5-(3-methyl-[1,2,4]triazolo [4,3-a]pyridin-6-yl)-7H-pyrrolo [2,3-d]pyrimidin-2-yl)amino)-1-methylcyclohexyl)acetamide [2983];
(1r,4r)-4-((4-methoxy-5-(3-methyl-[1,2,4]triazolo [4,3-a]pyridin-6-yl)-7H-pyrrolo [2,3-d]pyrimidin-2-yl)amino)-1-methylcyclohexan-1-ol [2984];
(1s,4s)-4-((4-methoxy-5-(3-methyl-[1,2,4]triazolo [4,3-a]pyridin-6-yl)-7H-pyrrolo [2,3-d]pyrimidin-2-yl)amino)-1-methylcyclohexan-1-ol [2985];
(1s,4s)-1-ethyl-4-((4-methoxy-5-(3-methyl-[1,2,4]triazolo [4,3-a]pyridin-6-yl)-7H-pyrrolo [2,3-d]pyrimidin-2-yl)amino)cyclohexan-1-ol [2986];
(1r,40-4-44-(methoxy-d₃)-5-(3-methyl41,2,41triazolo [4,3-a]pyridin-6-yl)-7H-pyrrolo [2,3-d]pyrimidin-2-yl)amino)-1-methylcyclohexan-1-ol [2987];
(1s,4s)-4-44-(methoxy-d₃)-5-(3-methyl41,2,41triazolo [4,3-a]pyridin-6-yl)-7H-pyrrolo [2,3-d]pyrimidin-2-yl)amino)-1-methylcyclohexan-1-ol [2988];
1-(4-((4-methoxy-5-(3-methyl-[1,2,4]triazolo [4,3-a]pyridin-6-yl)-7H-pyrrolo [2,3-d]pyrimidin-2-yl)amino)piperidin-1-yl)ethan-1-one [2989];
(S)-5-((4-methoxy-5-(3-methyl-[1,2,4]triazolo [4,3-a]pyridin-6-yl)-7H-pyrrolo [2,3-d]pyrimidin-2-yl)amino)-1-methylpiperidin-2-one [2990];
(R)-5-((4-methoxy-5-(3-methyl-[1,2,4]triazolo [4,3-a]pyridin-6-yl)-7H-pyrrolo [2,3-d]pyrimidin-2-yl)amino)-1-methylpiperidin-2-one [2991];
1-(2-((4-methoxy-5-(3-methyl-[1,2,4]triazolo [4,3-a]pyridin-6-yl)-7H-pyrrolo [2,3-d]pyrimidin-2-yl)amino)-7-azaspiro [3 .5]nonan-7-yl)ethan-1-one [2992];
1-((3aR,5s,6aS)-5-((4-methoxy-5-(3-methyl-[1,2,4]triazolo [4,3-a]pyridin-6-yl)-7H-pyrrolo [2,3-d]pyrimidin-2-yl)amino)hexahydrocyclopenta[c]pyrrol-2(1H)-yl)ethan-1-one [2993];
1-((3aR,5r,6aS)-5-((4-methoxy-5-(3-methyl-[1,2,4]triazolo [4,3-a]pyridin-6-yl)-7H-pyrrolo [2,3-d]pyrimidin-2-yl)amino)hexahydrocyclopenta[c]pyrrol-2(1H)-yl)ethan-1-one [2994];
1-(7-((4-methoxy-5-(3-methyl-[1,2,4]triazolo [4,3-a]pyridin-6-yl)-7H-pyrrolo [2,3-d]pyrimidin-2-yl)amino)-2-azaspiro[3.5]nonan-2-yl)ethan-1-one [2995];
4-methoxy-5-(3-methyl-[1,2,4]triazolo [4,3-a] pyridin-6-yl)-N-(2-oxaspiro [3 .5]nonan-7-yl)-7H-pyrrolo [2,3-d]pyrimidin-2-amine [2996];
4-methoxy-5-(3-methyl-[1,2,4]triazolo [4,3-a] pyridin-6-yl)-N-(1,4-dioxaspiro [4 .5] decan-8-yl)-7H-pyrrolo [2,3-d]pyrimidin-2-amine [2997];
(1s,3s)-3-45-([1,2,4]triazolo[1,5-a]pyridin-6-yl)-7H-pyrrolo[2,3-d]pyrimidin-2-yl)amino)-N,N,1-trimethylcyclobutane-1 -carboxamide [2998];
(1r,3r)-3-((5-([1,2,4]triazolo[1,5-a]pyridin-6-yl)-7H-pyrrolo[2,3-d]pyrimidin-2-yl)amino)-N,N,1-trimethylcyclobutane-1-carboxamide [2999];
41r,3r)-3-((5-([1,2,4]triazolo[1,5-a]pyridin-6-yl)-7H-pyrrolo[2,3-d]pyrimidin-2-yl)amino)-1-methylcyclobutyl)(pyrrolidin-1-yl)methanone [3000]; or a pharmaceutically acceptable salt thereof.

29. The compound of claim 1, wherein the compound of Formula I is selected from the group consisting of:
((1s,3 s)-3-((5-([1,2,4] triazolo [1,5-a] pyridin-6-yl)-7H-pyrrolo [2,3-d] pyrimidin-2-yl)amino)-1-methylcyclobutyl)(pyrrolidin-1-yl)methanone [3001];
N-((1s,3s)-3-((5-([1,2,4]triazolo [1,5-a] pyridin-6-yl)-7H-pyrrolo [2,3-d] pyrimidin-2-yl)amino)-1-methylcyclobutyl)acetamide [3002];
N-((1r,3r)-3-((5-([1,2,4]triazolo [1,5-a] pyridin-6-yl)-7H-pyrrolo [2,3-d] pyrimidin-2-yl)amino)-1-methylcyclobutypacetamide [3003];
N-((1s,3s)-3-((5-([1,2,4]triazolo [1,5-a] pyridin-6-yl)-7H-pyrrolo [2,3-d] pyrimidin-2-yl) amino)-1-methylcyclobutyl)propionamide [3004];
N-((1r,3r)-3-((5-([1,2,4]triazolo [1,5-a] pyridin-6-yl)-7H-pyrrolo [2,3-d] pyrimidin-2-yl)amino)-1-methylcyclobutyl)propionamide [3005];
1-((1r,3r)-3-((5-([1,2,4]triazolo[1,5-a]pyridin-6-yl)-7H-pyrrolo[2,3-d]pyrimidin-2-yl)amino)-1-methylcyclobutyl)pyrrolidin-2-one [3006];
1-((1s,3s)-3-((5-([1,2,4]triazolo [1,5-a] pyridin-6-yl)-7H-pyrrolo [2,3-d] pyrimidin-2-yl) amino)-1-methylcyclobutyl)pyrrolidin-2-one [3007];
(1s,3 s)-3-([1,2,4]triazolo [1,5-a]pyridin-6-yl)-7H-pyrrolo [2,3-d]pyrimidin-2-yl)amino)-1-methylcyclobutan-1-ol [3008];
(1r,3r)-3-((5-([1,2,4]triazolo[1,5-a]pyridin-6-yl)-7H-pyrrolo[2,3-d]pyrimidin-2-yl)amino)-1-methylcyclobutan-1-ol [3009];
(1s,4s)-4-45-([1,2,4]triazolo[1,5-a]pyridin-6-yl)-7H-pyrrolo[2,3-d]pyrimidin-2-yl)amino)-N,N-dimethylcyclohexane-1-carboxamide [3010];
((1s,4s)-4-((5-([1,2,4]triazolo[1,5-a]pyridin-6-yl)-7H-pyrrolo[2,3-d]pyrimidin-2-yl)amino)cyclohexyl)(pyrrolidin-1-yl)methanone [3011];
N-((1s,4s)-4-45-([1,2,4]triazolo[1,5-a]pyridin-6-yl)-7H-pyrrolo[2,3-d]pyrimidin-2-yl)amino)cyclohexypacetamide [3012];
N-((1r,4r)-4-((5-([1,2,4]triazolo[1,5-a]pyridin-6-yl)-7H-pyrrolo[2,3-d]pyrimidin-2-yl)amino)cyclohexypacetamide [3013];
1-((1s,4s)-4-45-([1,2,4]triazolo[1,5-a]pyridin-6-yl)-7H-pyrrolo[2,3-d]pyrimidin-2-yl)amino)cyclohexyl)pyrrolidin-2-one [3014];
2-(((1s,4s)-4-45-([1,2,4]triazolo[1,5-a]pyridin-6-yl)-7H-pyrrolo[2,3-d]pyrimidin-2-yl)amino)cyclohexyl)oxy)ethan-1-ol [3015];
2-(((1r,4r)-4-((5-([1,2,4]triazolo[1,5-a]pyridin-6-yl)-7H-pyrrolo[2,3-d]pyrimidin-2-yl)amino)cyclohexyl)oxy)ethan-1-ol [3016];
N-((1r,40-4-45-([1,2,4]triazolo[1,5-a]pyridin-6-yl)-7H-pyrrolo[2,3-d]pyrimidin-2-yl)amino)-1-methylcyclohexyl)acetamide [3017];
(1s,4s)-4-45-([1,2,4]triazolo[1,5-a]pyridin-6-yl)-7H-pyrrolo[2,3-d]pyrimidin-2-yl)amino)-1-methylcyclohexan-1-ol [3018];
(1r,4r)-4-((5-([1,2,4]triazolo[1,5-a]pyridin-6-yl)-7H-pyrrolo[2,3-d]pyrimidin-2-yl)amino)-1-methylcyclohexan-1-ol [3019];
(1s,4s)-4-45-([1,2,4]triazolo[1,5-a]pyridin-6-yl)-7H-pyrrolo[2,3-d]pyrimidin-2-yl)amino)-1-ethylcyclohexan-1-ol [3020];
1-(4-45-([1,2,4]triazolo[1,5-a]pyridin-6-yl)-7H-pyrrolo[2,3-d]pyrimidin-2-yl)amino)piperidin-1-yl)ethan-1-one [3021];
(S)-5-((5-([1,2,4]triazolo[1,5-a]pyridin-6-yl)-7H-pyrrolo[2,3-d]pyrimidin-2-yl)amino)-1-methylpiperidin-2-one [3022];
(R)-5-45-([1,2,4]triazolo[1,5-a]pyridin-6-yl)-7H-pyrrolo[2,3-d]pyrimidin-2-yl)amino)-1-methylpiperidin-2-one [3023];
1-(2-45-([1,2,4]triazolo[1,5-a]pyridin-6-yl)-7H-pyrrolo[2,3-d]pyrimidin-2-yl)amino)-7-azaspiro[3.5]nonan-7-yl)ethan-1-one [3024];
1-((3aR,5r,6aS)-5-((5-([1,2,4]triazolo[1,5-a]pyridin-6-yl)-7H-pyrrolo[2,3-d]pyrimidin-2-yl)amino)hexahydrocyclopenta[c]pyrrol-2(1H)-yl)ethan-1-one [3025];
1-((3aR,5s,6aS)-5-45-([1,2,4]triazolo[1,5-a]pyridin-6-yl)-7H-pyrrolo[2,3-d]pyrimidin-2-yl)amino)hexahydrocyclopenta[c]pyrrol-2(1H)-yl)ethan-1-one [3026];
1474(5-([1,2,4]triazolo [1,5-a]pyridin-6-yl)-7H-pyrrolo [2,3-d]pyrimidin-2-yl)amino)-2-azaspiro [3 .5]nonan-2-yl)ethan-1-one [3027];
5-([1,2,4]triazolo [1,5-a]pyridin-6-yl)-N-(2-oxaspiro [3 .5] nonan-7-yl)-7H-pyrrolo [2,3-d]pyrimidin-2-amine [3028];
5-([1,2,4]triazolo [1,5-a]pyridin-6-yl)-N-(1,4-dioxaspiro [4.5] decan-8-yl)-7H-pyrrolo [2,3-d]pyrimidin-2-amine [3029];
(1s,3s)-3-((5-([1,2,4]triazolo [1,5-a]pyridin-6-yl)-4-methoxy-7H-pyrrolo [2,3-d]pyrimidin-2-yl)amino)-N,N,1-trimethylcyclobutane-1-carboxamide [3030];
(1r,3r)-3-((5-([1,2,4]triazolo [1,5-a] pyridin-6-yl)-4-methoxy-7H-pyrrolo [2,3-d] pyrimidin-2-yl)amino)-N,N,1-trimethylcyclobutane-1-carboxamide [3031];
((1r,3r)-3-((5-([1,2,4]triazolo [1,5-a]pyridin-6-yl)-4-methoxy-7H-pyrrolo [2,3-d]pyrimidin-2-yl)amino)-1-methylcyclobutyl)(pyrrolidin-1-yl)methanone [3032];
((1s,3s)-3-((5-([1,2,4]triazolo [1,5-a]pyridin-6-yl)-4-methoxy-7H-pyrrolo [2,3-d]pyrimidin-2-yl)amino)-1-methylcyclobutyl)(pyrrolidin-1-yl)methanone [3033];
N-((1s,3s)-3-((5-([1,2,4]triazolo [1,5-a]pyridin-6-yl)-4-methoxy-7H-pyrrolo [2,3-d] pyrimidin-2-yl)amino)-1-methylcyclobutypacetamide [3034];
N-((1r,3r)-3-((5-([1,2,4]triazolo [1,5-a]pyridin-6-yl)-4-methoxy-7H-pyrrolo [2,3-d] pyrimidin-2-yl)amino)-1-methylcyclobutypacetamide [3035];
N-((1s,3s)-3-((5-([1,2,4]triazolo [1,5-a]pyridin-6-yl)-4-methoxy-7H-pyrrolo [2,3-d] pyrimidin-2-yl)amino)-1-methylcyclobutyl)propionamide [3036];
N-((1r,3r)-3-((5-([1,2,4]triazolo [1,5-a]pyridin-6-yl)-4-methoxy-7H-pyrrolo [2,3-d] pyrimidin-2-yl)amino)-1-methylcyclobutyl)propionamide [3037];
1-((1s,3s)-3-((5-([1,2,4]triazolo [1,5-a]pyridin-6-yl)-4-methoxy-7H-pyrrolo [2,3-d] pyrimidin-2-yl)amino)-1-methylcyclobutyl)pyrrolidin-2-one [3038];
1-((1r,3r)-3-((5-([1,2,4]triazolo [1,5-a]pyridin-6-yl)-4-methoxy-7H-pyrrolo [2,3-d]pyrimidin-2-yl)amino)-1-methylcyclobutyl)pyrrolidin-2-one [3039];
(1s,3s)-3-((5-([1,2,4]triazolo [1,5-a]pyridin-6-yl)-4-methoxy-7H-pyrrolo [2,3-d]pyrimidin-2-yl)amino)-1-methylcyclobutan-1-ol [3040];
(1r,3r)-3-((5-([1,2,4]triazolo [1,5-a] pyridin-6-yl)-4-methoxy-7H-pyrrolo [2,3-d] pyrimidin-2-yl)amino)-1-methylcyclobutan-1-ol [3041];
(1s,4s)-4-((5-([1,2,4]triazolo [1,5-a]pyridin-6-yl)-4-methoxy-7H-pyrrolo [2,3-d]pyrimidin-2-yl)amino)-N,N-dimethylcyclohexane-1-carboxamide [3042];
((1s,4s)-4-((5-([1,2,4]triazolo [1,5-a]pyridin-6-yl)-4-methoxy-7H-pyrrolo [2,3-d]pyrimidin-2-yl)amino)cyclohexyl)(pyrrolidin-1-yl)methanone [3043];
N-((1s,4s)-4-((5-([1,2,4]triazolo [1,5-a]pyridin-6-yl)-4-methoxy-7H-pyrrolo [2,3-d] pyrimidin-2-yl)amino)cyclohexyl)acetamide [3044];
N-((1r,4r)-4-((5-([1,2,4]triazolo [1,5-a]pyridin-6-yl)-4-methoxy-7H-pyrrolo [2,3-d] pyrimidin-2-yl)amino)cyclohexyl)acetamide [3045];
1-((1s,4s)-4-((5-([1,2,4]triazolo [1,5-a]pyridin-6-yl)-4-methoxy-7H-pyrrolo [2,3-d] pyrimidin-2-yl)amino)cyclohexyl)pyrrolidin-2-one [3046];
5-([1,2,4]triazolo [1,5-a]pyridin-6-yl)-4-methoxy-N-((1s,4s)-4-methoxycyclohexyl)-7H-pyrrolo [2,3-d]pyrimidin-2-amine [3047];
5-([1,2,4]triazolo [1,5-a]pyridin-6-yl)-4-(methoxy-d₃)-N-((1s,4s)-4-methoxycyclohexyl)-7H-pyrrolo [2,3-d]pyrimidin-2-amine [3048];
2-(((1s,4s)-4-45-([1,2,4]triazolo [1,5-a] pyridin-6-yl)-4-methoxy-7H-pyrrolo [2,3-d] pyrimidin-2-yl)amino)cyclohexyl)oxy)ethan-1-ol [3049];
2-(((1r,4r)-4-((5-([1,2,4]triazolo [1,5-a]pyridin-6-yl)-4-methoxy-7H-pyrrolo [2,3-d] pyrimidin-2-yl)amino)cyclohexyl)oxy)ethan-1-ol [3050];
N-((1r,4r)-4-((5-([1,2,4]triazolo [1,5-a]pyridin-6-yl)-4-methoxy-7H-pyrrolo [2,3-d] pyrimidin-2-yl)amino)-1-methylcyclohexyl)acetamide [3051];
(1r,4r) -44(54 [1,2,4]triazolo [1,5-a] pyridin-6-yl)-4-methoxy-7H-pyrrolo [2,3-d] pyrimidin-2-yl)amino)-1-methylcyclohexan-1-ol [3052];
(1s,4s)-4-((5-([1,2,4]triazolo [1,5-a]pyridin-6-yl)-4-methoxy-7H-pyrrolo [2,3-d]pyrimidin-2-yl)amino)-1-methylcyclohexan-1-ol [3053];
(1r,4r)-4-((5-([1,2,4]triazolo [1,5-a] pyridin-6-yl)-4-(methoxy-d₃)-7H-pyrrolo [2,3-d] pyrimidin-2-yl)amino)-1-methylcyclohexan-1-ol [3054];
(1s,4s)-4-((5-([1,2,4]triazolo [1,5-a]pyridin-6-yl)-4-(methoxy-d₃)-7H-pyrrolo [2,3-d]pyrimidin-2-yl)amino)-1-methylcyclohexan-1-ol [3055];
(1s,4s)-4-((5-([1,2,4]triazolo [1,5-a]pyridin-6-yl)-4-methoxy-7H-pyrrolo [2,3-d]pyrimidin-2-yl)amino)-1-ethylcyclohexan-1-ol [3056];
1-(4-((5-([1,2,4]triazolo [1,5-a]pyridin-6-yl)-4-methoxy-7H-pyrrolo [2,3-d]pyrimidin-2-yl)amino)piperidin-1-yl)ethan-1-one [3057];
(R)-5-((5-([1,2,4]triazolo [1,5-a]pyridin-6-yl)-4-methoxy-7H-pyrrolo [2,3-d]pyrimidin-2-yl)amino)-1-methylpiperidin-2-one [3058];
(S)-5-((5-([1,2,4]triazolo [1,5-a]pyridin-6-yl)-4-methoxy-7H-pyrrolo [2,3-d] pyrimidin-2-yl)amino)-1-methylpiperidin-2-one [3059];
1-(2-((5-([1,2,4]triazolo [1,5-a]pyridin-6-yl)-4-methoxy-7H-pyrrolo [2,3-d]pyrimidin-2-yl)amino)-7-azaspiro [3 .5] nonan-7-yl)ethan-1-one [3060];
1-((3aR,5s,6aS)-5-45-([1,2,4]triazolo [1,5-a]pyridin-6-yl)-4-methoxy-7H-pyrrolo [2,3-d]pyrimidin-2-yl)amino)hexahydrocyclopenta [c]pyrrol-2(1H)-yl)ethan-1-one [3061];
1-((3aR,5r,6aS)-5-((5-([1,2,4]triazolo [1,5-a]pyridin-6-yl)-4-methoxy-7H-pyrrolo [2,3-d]pyrimidin-2-yl)amino)hexahydrocyclopenta [c]pyrrol-2(1H)-yl)ethan-1-one [3062];
1-(7-((5-([1,2,4]triazolo [1,5-a]pyridin-6-yl)-4-methoxy-7H-pyrrolo [2,3-d]pyrimidin-2-yl)amino)-2-azaspiro [3 .5] nonan-2-yl)ethan-1-one [3063];
5-([1,2,4]triazolo [1,5-a]pyridin-6-yl)-4-methoxy-N-(2-oxaspiro [3 .5]nonan-7-yl)-7H-pyrrolo [2,3-d]pyrimidin-2-amine [3064];
5-([1,2,4]triazolo [1,5-a]pyridin-6-yl)-4-methoxy-N-(1,4-dioxaspiro [4 .5] de can-8-yl)-7H-pyrrolo [2,3-d]pyrimidin-2-amine [3065];
(1s,3s)-N,N,1-trimethyl-3-((5-(1-methyl-1H-benzo [d] [1,2,3]triazol-6-yl)-7H-pyrrolo [2,3-d]pyrimidin-2-yl)amino)cyclobutane-1-carboxamide [3066];
(1r,30-N,N,1-trimethyl-3-((5-(1-methyl-1H-benzo [d] [1,2,3]triazol-6-yl)-7H-pyrrolo [2,3-d]pyrimidin-2-yl)amino)cyclobutane-1-carboxamide [3067];
((1r,3r)-1-methyl-3-((5-(1-methyl-1H-benzo [d] [1,2,3]triazol-6-yl)-7H-pyrrolo [2,3-d]pyrimidin-2-yl)amino)cyclobutyl)(pyrrolidin-1-yl)methanone [3068];
((1s,3s)-1-methyl-3-((5-(1-methyl-1H-benzo[d] [1,2,3]triazol-6-yl)-7H-pyrrolo [2,3-d]pyrimidin-2-yl)amino)cyclobutyl)(pyrrolidin-1-yl)methanone [3069];
N-((1s,3s)-1-methyl-3-((5-(1-methyl-1H-benzo [d] [1,2,3]triazol -6-yl)-7H-pyrrolo [2,3-d]pyrimidin-2-yl)amino)cyclobutypacetamide [3070];
N-((1r,3r)-1-methyl-3-((5-(1-methyl-1H-benzo [d] [1,2,3]triazol -6-yl)-7H-pyrrolo [2,3-d]pyrimidin-2-yl)amino)cyclobutypacetamide [3071];
N-((1s,3s)-1-methyl-3-((5-(1-methyl-1H-benzo [d] [1,2,3]triazol-6-yl)-7H-pyrrolo [2,3-d]pyrimidin-2-yl)amino)cyclobutyl)propionamide [3072];
N-((1r,3r)-1-methyl-3-((5-(1-methyl-1H-benzo [d] [1,2,3]triazol -6-yl)-7H-pyrrolo [2,3-d]pyrimidin-2-yl)amino)cyclobutyl)propionamide [3073];
1-((1r,3r)-1-methyl-3-((5-(1-methyl-1H-benzo[d] [1,2,3]triazol-6-yl)-7H-pyrrolo [2,3-d]pyrimidin-2-yl)amino)cyclobutyppyrrolidin-2-one [3074];
1-((1s,3s)-1-methyl-3-((5-(1-methyl-1H-benzo[d] [1,2,3]triazol-6-yl)-7H-pyrrolo [2,3-d]pyrimidin-2-yl)amino)cyclobutyppyrrolidin-2-one [3075];
(1r,3r)-1-methyl-3-((5-(1-methyl-1H-benzo [d] [1,2,3]triazol-6-yl)-7H-pyrrolo [2,3-d]pyrimidin-2-yl)amino)cyclobutan-1-ol [3076];
(1s,3s)-1-methyl-3-((5-(1-methyl-1H-benzo [d] [1,2,3]triazol-6-yl)-7H-pyrrolo [2,3-d]pyrimidin-2-yl)amino)cyclobutan-1-ol [3077];
(1s,4s)-N,N-dimethyl-4-45-(1-methyl-1H-benzo[d][1,2,3]triazol-6-yl)-7H-pyrrolo[2,3-d]pyrimidin-2-yl)amino)cyclohexane-1-carboxamide [3078];
((1s,4s)-4-45-(1-methyl-1H-benzo[d][1,2,3]triazol-6-yl)-7H-pyrrolo[2,3-d]pyrimidin-2-yl)amino)cyclohexyl)(pyrrolidin-1-yl)methanone [3079];
N-((1r,4r)-4-((5-(1-methyl-1H-benzo[d][1,2,3]triazol-6-yl)-7H-pyrrolo[2,3-d]pyrimidin-2-yl)amino)cyclohexypacetamide [3080];
N-((1s,4s)-4-((5-(1-methyl-1H-benzo[d][1,2,3]triazol-6-yl)-7H-pyrrolo[2,3-d]pyrimidin-2-yl)amino)cyclohexypacetamide [3081];
1-((1s,4s)-4-((5-(1-methyl-1H-benzo[d][1,2,3]triazol-6-yl)-7H-pyrrolo[2,3-d]pyrimidin-2-yl)amino)cyclohexyl)pyrrolidin-2-one [3082];
2-(((1s,4s)-4-((5-(1-methyl-1H-benzo[d][1,2,3]triazol-6-yl)-7H-pyrrolo[2,3-d]pyrimidin-2-yl)amino)cyclohexyl)oxy)ethan-1-ol [3083];
2-(((1r,4r)-4-((5-(1-methyl-1H-benzo[d][1,2,3]triazol-6-yl)-7H-pyrrolo[2,3-d]pyrimidin-2-yl)amino)cyclohexyl)oxy)ethan-1-ol [3084];
N-((1r,4r)-1-methyl-4-((5-(1-methyl-1H-benzo[d][1,2,3]triazol-6-yl)-7H-pyrrolo[2,3-d]pyrimidin-2-yl)amino)cyclohexypacetamide [3085];
(1r,4r)-1-methyl-4-((5-(1-methyl-1H-benzo[d][1,2,3]triazol-6-yl)-7H-pyrrolo[2,3-d]pyrimidin-2-yl)amino)cyclohexan-1-ol [3086];
(1s,4s)-1-ethyl-4-((5-(1-methyl-1H-benzo[d][1,2,3]triazol-6-yl)-7H-pyrrolo[2,3-d]pyrimidin-2-yl)amino)cyclohexan-1-ol [3087];
1-(4-((5-(1-methyl-1H-benzo[d][1,2,3]triazol-6-yl)-7H-pyrrolo[2,3-d]pyrimidin-2-yl)amino)piperidin-1-yl)ethan-1-one [3088];
(S)-1-methyl-5-((5-(1-methyl-1H-benzo[d][1,2,3]triazol-6-yl)-7H-pyrrolo[2,3-d]pyrimidin-2-yl)amino)piperidin-2-one [3089];
(R)-1-methyl-5-((5-(1-methyl-1H-benzo[d][1,2,3]triazol-6-yl)-7H-pyrrolo[2,3-d]pyrimidin-2-yl)amino)piperidin-2-one [3090];
1-(2-((5-(1-methyl-1H-benzo[d][1,2,3]triazol-6-yl)-7H-pyrrolo[2,3-d]pyrimidin-2-yl)amino)-7-azaspiro [3 .5]nonan-7-yl)ethan-1-one [3091];
1-((3aR,5s,6aS)-5-((5-(1-methyl-1H-benzo[d][1,2,3]triazol-6-yl)-7H-pyrrolo[2,3-d]pyrimidin-2-yl)amino)hexahydrocyclopenta[c]pyrrol- 2(1H)-yl)ethan-1-one [3092];
1-((3aR,5r,6aS)-5-((5-(1-methyl-1H-benzo[d][1,2,3]triazol-6-yl)-7H-pyrrolo[2,3-d]pyrimidin-2-yl)amino)hexahydrocyclopenta[c]pyrrol- 2(1H)-yl)ethan-1-one [3093];
1-(7-((5-(1-methyl-1H-benzo[d][1,2,3]triazol-6-yl)-7H-pyrrolo[2,3-d]pyrimidin-2-yl)amino)-2-azaspiro[3.5]nonan-2-yl)ethan-1-one [3094];
5-(1-methyl-1H-benzo[d][1,2,3]triazol-6-yl)-N-(2-oxaspiro[3.5]nonan-7-yl)-7H-pyrrolo[2,3-d]pyrimidin-2-amine [3095];
5-(1-methyl-1H-benzo[d][1,2,3]triazol-6-yl)-N-(1,4-dioxaspiro[4.5]decan-8-yl)-7H-pyrrolo[2,3-d]pyrimidin-2-amine [3096];
(1s,3s)-3-((4-methoxy-5-(1-methyl-1H-benzo[d][1,2,3]triazol-6-yl)-7H-pyrrolo[2,3-d]pyrimidin-2-yl)amino)-N,N,1-trimethylcyclobutane-1-carboxamide [3097];
(1r,3r)-3-((4-methoxy-5-(1-methyl-1H-benzo[d][1,2,3]triazol-6-yl)-7H-pyrrolo[2,3-d]pyrimidin-2-yl)amino)-N,N,1-trimethylcyclobutane-1-carboxamide [3098];
((1r,3r)-3-((4-methoxy-5-(1-methyl-1H-benzo[d][1,2,3]triazol-6-yl)-7H-pyrrolo[2,3-d]pyrimidin-2-yl)amino)-1-methylcyclobutyl)(pyrrolidin-1-yl)methanone [3099];
((1s,3s)-3-((4-methoxy-5-(1-methyl-1H-benzo[d][1,2,3]triazol-6-yl)-7H-pyrrolo[2,3-d]pyrimidin-2-yl)amino)-1-methylcyclobutyl)(pyrrolidin-1-yl)methanone [3100];
N-((1s,3s)-3-44-methoxy-5-(1-methyl-1H-benzo[d][1,2,3]triazol-6-yl)-7H-pyrrolo[2,3-d]pyrimidin-2-yl)amino)-1-methylcyclobutyl)acetamide [3101];
N-((1r,3r)-3-44-methoxy-5-(1-methyl-1H-benzo[d][1,2,3]triazol-6-yl)-7H-pyrrolo[2,3-d]pyrimidin-2-yl)amino)-1-methylcyclobutyl)acetamide [3102];
N-((1s,3s)-3-44-methoxy-5-(1-methyl-1H-benzo[d][1,2,3]triazol-6-yl)-7H-pyrrolo[2,3-d]pyrimidin-2-yl)amino)-1-methylcyclobutyl)propionamide [3103];
N-((1r,3r)-3-44-methoxy-5-(1-methyl-1H-benzo[d][1,2,3]triazol-6-yl)-7H-pyrrolo[2,3-d]pyrimidin-2-yl)amino)-1-methylcyclobutyl)propionamide [3104];
1-((1r,3r)-3-((4-methoxy-5-(1-methyl-1H-benzo[d][1,2,3]triazol-6-yl)-7H-pyrrolo[2,3-d]pyrimidin-2-yl)amino)-1-methylcyclobutyl)pyrrolidin-2-one [3105];
1-((1s,3s)-3-44-methoxy-5-(1-methyl-1H-benzo[d][1,2,3]triazol-6-yl)-7H-pyrrolo[2,3-d]pyrimidin-2-yl)amino)-1-methylcyclobutyl)pyrrolidin-2-one [3106];
(1s,3s)-3-((4-methoxy-5-(1-methyl-1H-benzo[d][1,2,3]triazol-6-yl)-7H-pyrrolo[2,3-d]pyrimidin-2-yl)amino)-1-methylcyclobutan-1-ol [3107];
(1r,3r)-3-((4-methoxy-5-(1-methyl-1H-benzo[d][1,2,3]triazol-6-yl)-7H-pyrrolo[2,3-d]pyrimidin-2-yl)amino)-1-methylcyclobutan-1-ol [3108];
(1s,4s)-4-((4-methoxy-5-(1-methyl-1H-benzo[d][1,2,3]triazol-6-yl)-7H-pyrrolo[2,3-d]pyrimidin-2-yl)amino)-N,N-dimethylcyclohexane-1-carboxamide [3109];
((1s,4s)-4-((4-methoxy-5-(1-methyl-1H-benzo[d][1,2,3]triazol-6-yl)-7H-pyrrolo[2,3-d]pyrimidin-2-yl)amino)cyclohexyl)(pyrrolidin-1-yl)methanone [3110];
N-((1s,4s)-4-44-methoxy-5-(1-methyl-1H-benzo[d][1,2,3]triazol-6-yl)-7H-pyrrolo[2,3-d]pyrimidin-2-yl)amino)cyclohexypacetamide [3111];
N-((1r,4r)-4-((4-methoxy-5-(1-methyl-1H-benzo[d][1,2,3]triazol-6-yl)-7H-pyrrolo[2,3-d]pyrimidin-2-yl)amino)cyclohexypacetamide [3112];
1-((1s,4s)-4-44-methoxy-5-(1-methyl-1H-benzo[d][1,2,3]triazol-6-yl)-7H-pyrrolo[2,3-d]pyrimidin-2-yl)amino)cyclohexyl)pyrrolidin-2-one [3113];
4-methoxy-N-((1s,4s)-4-methoxycyclohexyl)-5-(1-methyl-1H-benzo[d][1,2,3]triazol-6-yl)-7H-pyrrolo[2,3-d]pyrimidin-2-amine [3114];
4-(methoxy-d3)-N-((1s,4s)-4-methoxycyclohexyl)-5-(1-methyl-lH-benzo[d][1,2,3]triazol-6-yl)-7H-pyrrolo[2,3-d]pyrimidin-2-amine [3115];
2-(((1s,4s)-4-((4-methoxy-5-(1-methyl-1H-benzo[d][1,2,3]triazol-6-yl)-7H-pyrrolo[2,3-d]pyrimidin-2-yl)amino)cyclohexyl)oxy)ethan-1-ol [3116];
2-(((1r,4r)-4-((4-methoxy-5-(1-methyl-1H-benzo[d][1,2,3]triazol-6-yl)-7H-pyrrolo[2,3-d]pyrimidin-2-yl)amino)cyclohexyl)oxy)ethan-1-ol [3117];
N-((1r,4r)-4-((4-methoxy-5-(1-methyl-1H-benzo[d][1,2,3]triazol-6-yl)-7H-pyrrolo[2,3-d]pyrimidin-2-yl)amino)-1-methylcyclohexyl)acetamide [3118];
(1r,4r)-4-((4-methoxy-5-(1-methyl-1H-benzo[d][1,2,3]triazol-6-yl)-7H-pyrrolo[2,3-d]pyrimidin-2-yl)amino)-1-methylcyclohexan-1-ol [3119];
(1s,4s)-4-((4-methoxy-5-(1-methyl-1H-benzo[d][1,2,3]triazol-6-yl)-7H-pyrrolo[2,3-d]pyrimidin-2-yl)amino)-1-methylcyclohexan-1-ol [3120];
(1r,40-4-44-(methoxy-d₃)-5-(1-methyl-1H-benzo[d][1,2,3]triazol-6-yl)-7H-pyrrolo[2,3-d]pyrimidin-2-yl)amino)-1-methylcyclohexan-1-ol [3121];
(1s,4s)-4-44-(methoxy-d₃)-5-(1-methyl-1H-benzo[d][1,2,3]triazol-6-yl)-7H-pyrrolo[2,3-d]pyrimidin-2-yl)amino)-1-methylcyclohexan-1-ol [3122];
(1s,4s)-1-ethyl-4-((4-methoxy-5-(1-methyl-1H-benzo[d][1,2,3]triazol-6-yl)-7H-pyrrolo[2,3-d]pyrimidin-2-yl)amino)cyclohexan-1-ol [3123];
1-(4-((4-methoxy-5-(1-methyl-1H-benzo[d][1,2,3]triazol-6-yl)-7H-pyrrolo[2,3-d]pyrimidin-2-yl)amino)piperidin-1-yl)ethan-1-one [3124];
(R)-5-((4-methoxy-5-(1-methyl-1H-benzo[d][1,2,3]triazol-6-yl)-7H-pyrrolo[2,3-d]pyrimidin-2-yl)amino)-1-methylpiperidin-2-one [3125];
(S)-5-((4-methoxy-5-(1-methyl-1H-benzo[d][1,2,3]triazol-6-yl)-7H-pyrrolo[2,3-d]pyrimidin-2-yl)amino)-1-methylpiperidin-2-one [3126];
N-((1R,5S,6s)-3-oxabicyclo[3.1.0]hexan-6-yl)-4-methoxy-5-(1-methyl-1H-benzo[d][1,2,3]triazol-6-yl)-7H-pyrrolo[2,3-d]pyrimidin-2-amine [3127];
N-((1R,5S,60-3-oxabicyclo[3.1.0]hexan-6-yl)-4-methoxy-5-(1-methyl-1H-benzo[d][1,2,3]triazol-6-yl)-7H-pyrrolo[2,3-d]pyrimidin-2-amine [3128];
1-(2-((4-methoxy-5-(1-methyl-1H-benzo[d][1,2,3]triazol-6-yl)-7H-pyrrolo[2,3-d]pyrimidin-2-yl)amino)-7-azaspiro[3.5]nonan-7-yl)ethan-1-one [3129];
1-((3aR,5s,6aS)-5-((4-methoxy-5-(1-methyl-1H-benzo[d][1,2,3]triazol-6-yl)-7H-pyrrolo[2,3-d]pyrimidin-2-yl)amino)hexahydrocyclopenta[c]pyrrol-2(1H)-yl)ethan-1-one [3130];
1-((3aR,5r,6aS)-5-((4-methoxy-5-(1-methyl-1H-benzo[d][1,2,3]triazol-6-yl)-7H-pyrrolo[2,3-d]pyrimidin-2-yl)amino)hexahydrocyclopenta[c]pyrrol-2(1H)-yl)ethan-1-one [3131];
1-(7-((4-methoxy-5-(1-methyl-1H-benzo[d][1,2,3]triazol-6-yl)-7H-pyrrolo[2,3-d]pyrimidin-2-yl)amino)-2-azaspiro[3.5]nonan-2-yl)ethan-1-one [3132];
4-methoxy-5-(1-methyl-1H-benzo[d][1,2,3]triazol-6-yl)-N-(2-oxaspiro[3.5]nonan-7-yl)-7H-pyrrolo[2,3-d]pyrimidin-2-amine [3133];
4-methoxy-5-(1-methyl-1H-benzo[d][1,2,3]triazol-6-yl)-N-(1,4-dioxaspiro[4.5]decan-8-yl)-7H-pyrrolo[2,3-d]pyrimidin-2-amine [3134];
(1s,3s)-3-((5-(1-(2,2-difluoroethyl)-1H-benzo[d][1,2,3]triazol-6-yl)-7H-pyrrolo[2,3-d]pyrimidin-2-yl)amino)-N,N,1-trimethylcyclobutane-1-carboxamide [3135];
(1r,3r)-3-((5-(1-(2,2-difluoroethyl)-1H-benzo[d][1,2,3]triazol-6-yl)-7H-pyrrolo[2,3-d]pyrimidin-2-yl)amino)-N,N,1-trimethylcyclobutane-1-carboxamide [3136];
((1s,3s)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)(pyrrolidin-1-yl)methanone [3137];
((1r,3r)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)(pyrrolidin-1-yl)methanone [3138];
N-((1s,3s)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)acetamide [3139];
N-((1r,3r)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)acetamide [3140];
N-((1s,3s)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)propionamide [3141];
N-((1r,3r)-3-((3-(1-(2,2-difluoroethyl)-1H-benzo[d][1,2,3]triazol-6-yl)-112-pyrrolo[3,2-c]pyridin-6-yl)amino)-1-methylcyclobutyl)propionamide [3142];
1-((1r,3r)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)pyrrolidin-2-one [3143];
1-((1s,3s)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutyl)pyrrolidin-2-one [3144];
(1s,3s)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutan-1-ol [3145];
(1r,3r)-3-((5-(1-(2,2-difluoroethyl)-1H-benzo[d][1,2,3]triazol-6-yl)-7H-pyrrolo[2,3-d]pyrimidin-2-yl)amino)-1-methylcyclobutan-1-ol [3146];
(1s,4s)-4-((5-(1-(2,2-difluoroethyl)-1H-benzo[d][1,2,3]triazol-6-yl)-7H-pyrrolo[2,3-d]pyrimidin-2-yl)amino)-N,N-dimethylcyclohexane-1-carboxamide [3147];
((1s,4s)-4-((5-(1-(2,2-difluoroethyl)-1H-benzo[d][1,2,3]triazol-6-yl)-7H-pyrrolo[2,3-d]pyrimidin-2-yl)amino)cyclohexyl)(pyrrolidin-1-yl)methanone [3148];
N-((1s,4s)-4-((5-(1-(2,2-difluoroethyl)-1H-benzo[d][1,2,3]triazol-6-yl)-7H-pyrrolo[2,3-d]pyrimidin-2-yl)amino)cyclohexypacetamide [3149];
N-((1r,4r)-4-((5-(1-(2,2-difluoroethyl)-1H-benzo[d][1,2,3]triazol-6-yl)-7H-pyrrolo[2,3-d]pyrimidin-2-yl)amino)cyclohexypacetamide [3150];
1-((1s,4s)-4-((5-(1-(2,2-difluoroethyl)-1H-benzo[d][1,2,3]triazol-6-yl)-7H-pyrrolo[2,3-d]pyrimidin-2-yl)amino)cyclohexyl)pyrrolidin-2-one [3151];
5-(1-(2,2-difluoroethyl)-1H-benzo[d][1,2,3]triazol-6-yl)-N-((1s,4s)-4-methoxycyclohexyl)-7H-pyrrolo[2,3-d]pyrimidin-2-amine [3152];
2-(((1s,4s)-4-((5-(1-(2,2-difluoroethyl)-1H-benzo[d][1,2,3]triazol-6-yl)-7H-pyrrolo[2,3-d]pyrimidin-2-yl)amino)cyclohexyl)oxy)ethan-1-ol [3153];
2-(((1r,4r)-4-((5-(1-(2,2-difluoroethyl)-1H-benzo[d][1,2,3]triazol-6-yl)-7H-pyrrolo[2,3-d]pyrimidin-2-yl)amino)cyclohexyl)oxy)ethan-1-ol [3154];
N-((1r,4r)-4-((5-(1-(2,2-difluoroethyl)-1H-benzo[d][1,2,3]triazol-6-yl)-7H-pyrrolo[2,3-d]pyrimidin-2-yl)amino)-1-methylcyclohexyl)acetamide [3155];
(1s,4s)-4-((5-(1-(2,2-difluoroethyl)-1H-benzo[d][1,2,3]triazol-6-yl)-7H-pyrrolo[2,3-d]pyrimidin-2-yl)amino)-1-methylcyclohexan-1-ol [3156];
(1r,4r)-4-((5-(1-(2,2-difluoroethyl)-1H-benzo[d][1,2,3]triazol-6-yl)-7H-pyrrolo[2,3-d]pyrimidin-2-yl)amino)-1-methylcyclohexan-1-ol [3157];
(1s,4s)-4-((5-(1-(2,2-difluoroethyl)-1H-benzo[d][1,2,3]triazol-6-yl)-7H-pyrrolo[2,3-d]pyrimidin-2-yl)amino)-1-ethylcyclohexan-1-ol [3158];
1-(4-((5-(1-(2,2-difluoroethyl)-1H-benzo[d][1,2,3]triazol-6-yl)-7H-pyrrolo[2,3-d]pyrimidin-2-yl)amino)piperidin-1-yl)ethan-1-one [3159];
(S)-5-((5-(1-(2,2-difluoroethyl)-1H-benzo[d][1,2,3]triazol-6-yl)-7H-pyrrolo[2,3-d]pyrimidin-2-yl)amino)-1-methylpiperidin-2-one [3160];
(R)-5-((5-(1-(2,2-difluoroethyl)-1H-benzo[d][1,2,3]triazol-6-yl)-7H-pyrrolo[2,3-d]pyrimidin-2-yl)amino)-1-methylpiperidin-2-one [3161];
1-(2-((5-(1-(2,2-difluoroethyl)-1H-benzo[d][1,2,3]triazol-6-yl)-7H-pyrrolo[2,3-d]pyrimidin-2-yl)amino)-7-azaspiro[3.5]nonan-7-yl)ethan-1-one [3162];
1-((3aR,5s,6aS)-5-((5-(1-(2,2-difluoroethyl)-1H-benzo[d][1,2,3]triazol-6-yl)-7H-pyrrolo[2,3-d]pyrimidin-2-yl)amino)hexahydrocyclopenta[c]pyrrol-2(1H)-yl)ethan-1-one [3163];
1-((3aR,5r,6aS)-5-((5-(1-(2,2-difluoroethyl)-1H-benzo[d][1,2,3]triazol-6-yl)-7H-pyrrolo[2,3-d]pyrimidin-2-yl)amino)hexahydrocyclopenta[c]pyrrol-2(1H)-yl)ethan-1-one [3164];
1-(7-((5-(1-(2,2-difluoroethyl)-1H-benzo[d][1,2,3]triazol-6-yl)-7H-pyrrolo[2,3-d]pyrimidin-2-yl)amino)-2-azaspiro[3.5]nonan-2-yl)ethan-1-one [3165];
5-(1-(2,2-difluoroethyl)-1H-benzo[d][1,2,3]triazol-6-yl)-N-(2-oxaspiro[3.5]nonan-7-yl)-7H-pyrrolo[2,3-d]pyrimidin-2-amine [3166];
5-(1-(2,2-difluoroethyl)-1H-benzo[d][1,2,3]triazol-6-yl)-N-(1,4-dioxaspiro[4.5]decan-8-yl)-7H-pyrrolo[2,3-d]pyrimidin-2-amine [3167];
(1s,3s)-3-((5-(1-(2,2-difluoroethyl)-1H-benzo[d][1,2,3]triazol-6-yl)-4-methoxy-7H-pyrrolo[2,3-d]pyrimidin-2-yl)amino)-N,N,1-trimethylcyclobutane-1-carboxamide [3168];
(1r,3r)-3-((5-(1-(2,2-difluoroethyl)-1H-benzo[d][1,2,3]triazol-6-yl)-4-methoxy-7H-pyrrolo[2,3-d]pyrimidin-2-yl)amino)-N,N,1-trimethylcyclobutane-1-carboxamide [3169];
((1s,3s)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)(pyrrolidin-1-yl)methanone [3170];
((1r,3r)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)(pyrrolidin-1-yl)methanone [3171];
N-((1s,3s)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)acetamide [3172];
N-((1r,3r)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)acetamide [3173];
N-((1s,3s)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)propionamide [3174];
N-((1r,3r)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)propionamide [3175];
1-((1r,3r)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)pyrrolidin-2-one [3176];
1-((1s,3s)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutyl)pyrrolidin-2-one [3177];
(1s,3s)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutan-1-ol [3178];
(1r,3r)-3-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclobutan-1-ol [3179];
(1s,4s)-4-((5-(1-(2,2-difluoroethyl)-1H-benzo[d][1,2,3]triazol-6-yl)-4-methoxy-7H-pyrrolo[2,3-d]pyrimidin-2-yl)amino)-N,N-dimethylcyclohexane-1-carboxamide [3180];
((1s,4s)-4-((5-(1-(2,2-difluoroethyl)-1H-benzo[d][1,2,3]triazol-6-yl)-4-methoxy-7H-pyrrolo[2,3-d]pyrimidin-2-yl)amino)cyclohexyl)(pyrrolidin-1-yl)methanone [3181];
N-((1r,4r)-4-((5-(1-(2,2-difluoroethyl)-1H-benzo[d][1,2,3]triazol-6-yl)-4-methoxy-7H-pyrrolo[2,3-d]pyrimidin-2-yl)amino)cyclohexypacetamide [3182];
N-((1s,4s)-4-45-(1-(2,2-difluoroethyl)-1H-benzo[d][1,2,3]triazol-6-yl)-4-methoxy-7H-pyrrolo[2,3-d]pyrimidin-2-yl)amino)cyclohexypacetamide [3183];
1-((1s,4s)-4-45-(1-(2,2-difluoroethyl)-1H-benzo[d][1,2,3]triazol-6-yl)-4-methoxy-7H-pyrrolo[2,3-d]pyrimidin-2-yl)amino)cyclohexyl)pyrrolidin-2-one [3184];
5-(1-(2,2-difluoroethyl)-1H-benzo[d][1,2,3]triazol-6-yl)-4-methoxy-N-((1s,4s)-4-methoxycyclohexyl)-7H-pyrrolo[2,3-d]pyrimidin-2-amine [3185];
5-(1-(2,2-difluoroethyl)-1H-benzo[d][1,2,3]triazol-6-yl)-4-(methoxy-d₃)-N-((1s,4s)-4-methoxycyclohexyl)-7H-pyrrolo[2,3-d]pyrimidin-2-amine [3186];
2-(((1s,4s)-4-((5-(1-(2,2-difluoroethyl)-1H-benzo[d][1,2,3]triazol-6-yl)-4-methoxy-7H-pyrrolo[2,3-d]pyrimidin-2-yl)amino)cyclohexyl)oxy)ethan-1-ol [3187];
2-(((1r,4r)-4-((5-(1-(2,2-difluoroethyl)-1H-benzo[d][1,2,3]triazol-6-yl)-4-methoxy-7H-pyrrolo[2,3-d]pyrimidin-2-yl)amino)cyclohexyl)oxy)ethan-1-ol [3188];
N-((1r,4r)-4-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclohexypacetamide [3189];
(1r,4r)-4-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclohexan-1-ol [3190];
(1s,4s)-4-((5-(1-(2,2-difluoroethyl)-1H-benzo[d][1,2,3]triazol-6-yl)-4-methoxy-7H-pyrrolo[2,3-d]pyrimidin-2-yl)amino)-1-methylcyclohexan-1-ol [3191];
(1r,4r)-4-((5-(1-(2,2-difluoroethyl)-1H-benzo[d][1,2,3]triazol-6-yl)-4-(methoxy-d₃)-7H-pyrrolo[2,3-d]pyrimidin-2-yl)amino)-1-methylcyclohexan-1-ol [3192];
(1s,4s)-4-((5-(1-(2,2-difluoroethyl)-1H-benzo[d][1,2,3]triazol-6-yl)-4-(methoxy-d₃)-7H-pyrrolo[2,3-d]pyrimidin-2-yl)amino)-1-methylcyclohexan-1-ol [3193];
(1s,4s)-4-((5-(1-(2,2-difluoroethyl)-1H-benzo[d][1,2,3]triazol-6-yl)-4-methoxy-7H-pyrrolo[2,3-d]pyrimidin-2-yl)amino)-1-ethylcyclohexan-1-ol [3194];
1-(4-((5-(1-(2,2-difluoroethyl)-1H-benzo[d][1,2,3]triazol-6-yl)-4-methoxy-7H-pyrrolo[2,3-d]pyrimidin-2-yl)amino)piperidin-1-yl)ethan-1-one [3195];
(R)-5-((5-(1-(2,2-difluoroethyl)-1H-benzo[d][1,2,3]triazol-6-yl)-4-methoxy-7H-pyrrolo[2,3-d]pyrimidin-2-yl)amino)-1-methylpiperidin-2-one [3196];
(S)-5-((5-(1-(2,2-difluoroethyl)-1H-benzo[d][1,2,3]triazol-6-yl)-4-methoxy-7H-pyrrolo[2,3-d]pyrimidin-2-yl)amino)-1-methylpiperidin-2-one [3197];
1-(2-((5-(1-(2,2-difluoroethyl)-1H-benzo[d][1,2,3]triazol-6-yl)-4-methoxy-7H-pyrrolo[2,3-d]pyrimidin-2-yl)amino)-7-azaspiro[3.5]nonan-7-yl)ethan-1-one [3198];
1-((3aR,5r,6aS)-5-((5-(1-(2,2-difluoroethyl)-1H-benzo[d][1,2,3]triazol-6-yl)-4-methoxy-7H-pyrrolo[2,3-d]pyrimidin-2-yl)amino)hexahydrocyclopenta[c]pyrrol-2(1H)-yl)ethan-1-one [3199];
1-((3aR,5s,6aS)-5-((5-(1-(2,2-difluoroethyl)-1H-benzo[d][1,2,3]triazol-6-yl)-4-methoxy-7H-pyrrolo[2,3-d]pyrimidin-2-yl)amino)hexahydrocyclopenta[c]pyrrol-2(1H)-yl)ethan-1-one [3200];
1-(7-((5-(1-(2,2-difluoroethyl)-1H-benzo[d][1,2,3]triazol-6-yl)-4-methoxy-7H-pyrrolo[2,3-d]pyrimidin-2-yl)amino)-2-azaspiro[3.5]nonan-2-yl)ethan-1-one [3201];
5-(1-(2,2-difluoroethyl)-1H-benzo[d][1,2,3]triazol-6-yl)-4-methoxy-N-(2-oxaspiro[3.5]nonan-7-yl)-7H-pyrrolo[2,3-d]pyrimidin-2-amine [3202];
5-(1-(2,2-difluoroethyl)-1H-benzo[d][1,2,3]triazol-6-yl)-4-methoxy-N-(1,4-dioxaspiro[4.5]decan-8-yl)-7H-pyrrolo[2,3-d]pyrimidin-2-amine [3203];
(1s,3s)-3-((5-(imidazo[1,2-b]pyridazin-6-yl)-7H-pyrrolo[2,3-d]pyrimidin-2-yl)amino)-N,N,1-trimethylcyclobutane-1-carboxamide [3204];
(1r,3r)-3-((5-(imidazo[1,2-b]pyridazin-6-yl)-7H-pyrrolo[2,3-d]pyrimidin-2-yl)amino)-N,N,1-trimethylcyclobutane-1-carboxamide [3205];
((1r,3r)-3-((5-(imidazo[1,2-b]pyridazin-6-yl)-7H-pyrrolo[2,3-d]pyrimidin-2-yl)amino)-1-methylcyclobutyl)(pyrrolidin-1-yl)methanone [3206];
((1s,3s)-3-((5-(imidazo[1,2-b]pyridazin-6-yl)-7H-pyrrolo[2,3-d]pyrimidin-2-yl)amino)-1-methylcyclobutyl)(pyrrolidin-1-yl)methanone [3207];
N-((1s,3s)-3-((5-(imidazo[1,2-b]pyridazin-6-yl)-7H-pyrrolo[2,3-d]pyrimidin-2-yl)amino)-1-methylcyclobutypacetamide [3208];
N-((1r,3r)-3-((5-(imidazo[1,2-b]pyridazin-6-yl)-7H-pyrrolo[2,3-d]pyrimidin-2-yl)amino)-1-methylcyclobutypacetamide [3209];
N-((1s,3s)-3-((5-(imidazo[1,2-b]pyridazin-6-yl)-7H-pyrrolo[2,3-d]pyrimidin-2-yl)amino)-1-methylcyclobutyl)propionamide [3210];
N-((1r,3r)-3-((5-(imidazo[1,2-b]pyridazin-6-yl)-7H-pyrrolo[2,3-d]pyrimidin-2-yl)amino)-1-methylcyclobutyl)propionamide [3211];
1-((1r,3r)-3-((5-(imidazo[1,2-b]pyridazin-6-yl)-7H-pyrrolo[2,3-d]pyrimidin-2-yl)amino)-1-methylcyclobutyl)pyrrolidin-2-one [3212];
1-((1s,3s)-3-((5-(imidazo[1,2-b]pyridazin-6-yl)-7H-pyrrolo[2,3-d]pyrimidin-2-yl)amino)-1-methylcyclobutyl)pyrrolidin-2-one [3213];
(1s,3s)-3-((5-(imidazo[1,2-b]pyridazin-6-yl)-7H-pyrrolo[2,3-d]pyrimidin-2-yl)amino)-1-methylcyclobutan-1-ol [3214];
(1r,3r)-3-((5-(imidazo[1,2-b]pyridazin-6-yl)-7H-pyrrolo[2,3-d]pyrimidin-2-yl)amino)-1-methylcyclobutan-1-ol [3215];
(1s,4s)-4-((5-(imidazo[1,2-b]pyridazin-6-yl)-7H-pyrrolo[2,3-d]pyrimidin-2-yl)amino)-N,N-dimethylcyclohexane-1-carboxamide [3216];
((1s,4s)-4-((5-(imidazo[1,2-b]pyridazin-6-yl)-7H-pyrrolo[2,3-d]pyrimidin-2-yl)amino)cyclohexyl)(pyrrolidin-1-yl)methanone [3217];
N-((1s,4s)-4-((5-(imidazo[1,2-b]pyridazin-6-yl)-7H-pyrrolo[2,3-d]pyrimidin-2-yl)amino)cyclohexypacetamide [3218];
N-((1r,4r)-4-((5-(imidazo[1,2-b]pyridazin-6-yl)-7H-pyrrolo[2,3-d]pyrimidin-2-yl)amino)cyclohexypacetamide [3219];
1-((1s,4s)-4-((5-(imidazo[1,2-b]pyridazin-6-yl)-7H-pyrrolo[2,3-d]pyrimidin-2-yl)amino)cyclohexyl)pyrrolidin-2-one [3220];
2-(((1s,4s)-4-((5-(imidazo[1,2-b]pyridazin-6-yl)-7H-pyrrolo[2,3-d]pyrimidin-2-yl)amino)cyclohexyl)oxy)ethan-1-ol [3221];
2-(((1r,4r)-4-((5-(imidazo[1,2-b]pyridazin-6-yl)-7H-pyrrolo[2,3-d]pyrimidin-2-yl)amino)cyclohexyl)oxy)ethan-1-ol [3222];
N-((1r,40-4-45-(imidazo[1,2-b]pyridazin-6-yl)-7H-pyrrolo[2,3-d]pyrimidin-2-yl)amino)-1-methylcyclohexyl)acetamide [3223];
(1s,4s)-4-((5-(imidazo[1,2-b]pyridazin-6-yl)-7H-pyrrolo[2,3-d]pyrimidin-2-yl)amino)-1-methylcyclohexan-1-ol [3224];
(1r,40-4-45-(imidazo[1,2-b]pyridazin-6-yl)-7H-pyrrolo[2,3-d]pyrimidin-2-yl)amino)-1-methylcyclohexan-1-ol [3225];
(1s,4s)-1-ethyl-4-((5-(imidazo[1,2-b]pyridazin-6-yl)-7H-pyrrolo[2,3-d]pyrimidin-2-yl)amino)cyclohexan-1-01 [3226];
1-(4-((5-(imidazo[1,2-b]pyridazin-6-yl)-7H-pyrrolo[2,3-d]pyrimidin-2-yl)amino)piperidin-1-yl)ethan-1-one [3227];
(R)-5-((5-(imidazo[1,2-b]pyridazin-6-yl)-7H-pyrrolo[2,3-d]pyrimidin-2-yl)amino)-1-methylpiperidin-2-one [3228];
(S)-5-((5-(imidazo[1,2-b]pyridazin-6-yl)-7H-pyrrolo[2,3-d]pyrimidin-2-yl)amino)-1-methylpiperidin-2-one [3229];
1-(2-((5-(imidazo [1 ,2-b]pyridazin-6-yl)-7H-pyrrolo [2,3-d]pyrimidin-2-yl)amino)-7-azaspiro [3 .5]nonan-7-yl)ethan-1-one [3230];
1-((3 aR,5 s,6aS)-5-((5-(imidazo [1 ,2-b]pyridazin-6-yl)-7H-pyrrolo [2,3-d]pyrimidin-2-yl)amino)hexahydrocyclopenta[c]pyrrol-2(1 H)-yl)ethan-1-one [3231];
1-((3aR,5r,6aS)-5-((5-(imidazo [1 ,2-b]pyridazin-6-yl)-7H-pyrrolo [2,3-d]pyrimidin-2-yl)amino)hexahydrocyclopenta[c]pyrrol-2(1 H)-yl)ethan-1-one [3232];
1-(7-((5-(imidazo [1 ,2-b]pyridazin-6-yl)-7H-pyrrolo [2,3-d]pyrimidin-2-yl)amino)-2-azaspiro [3 .5]nonan-2-yl)ethan-1-one [3233];
5-(imidazo [1 ,2-b]pyridazin-6-yl)-N-(2-oxaspiro [3 .5]nonan-7-yl)-7H-pyrrolo [2,3-d]pyrimidin-2-amine [3234];
5-(imidazo [1 ,2-b]pyridazin-6-yl)-N-(1 ,4-dioxaspiro [4.5] decan-8-yl)-7H-pyrrolo [2,3-d]pyrimidin-2-amine [3235];
N²-(4,4-difluorocyclohexyl)-5-(imidazo [1 ,2-b]pyridazin-6-yl)-N⁴-methyl -7H-pyrrolo [2,3-d]pyrimidine-2,4-diamine [3236];
(1 s,4s)-4-((5-(imidazo[1 ,2-b]pyridazin-6-yl)-4-(methylamino)-7H-pyrrolo [2,3-d]pyrimidin-2-yl)amino)-N,N-dimethylcyclohexane-1-carboxamide [3237];
(1 r,40-4-45-(imidazo [1 ,2-b]pyridazin-6-yl)-4-(methylamino)-7H-pyrrolo [2,3-d]pyrimidin-2-yl)amino)-N,N-dimethylcyclohexane-1-carboxamide [3238];
((1 r,40-4-45-(imidazo[1 ,2-b]pyridazin-6-yl)-4-(methylamino)-7H-pyrrolo [2,3-d]pyrimidin-2-yl)amino)cyclohexyl)(pyrrolidin-1-yl)methanone [3239];
((1 s,4s)-4-((5-(imidazo [1 ,2-b]pyridazin-6-yl)-4-(methylamino)-7H-pyrrolo [2,3-d]pyrimidin-2-yl)amino)cyclohexyl)(pyrrolidin-1-yl)methanone [3240];
N-((1 s,4s)-4-((5-(imidazo [1 ,2-b]pyridazin-6-yl)-4-(methylamino)-7H-pyrrolo [2,3-d]pyrimidin-2-yl)amino)cyclohexypacetamide [3241];
N-((1 r,40-4-45-(imidazo [1 ,2-b]pyridazin-6-yl)-4-(methylamino)-7H-pyrrolo [2,3-d]pyrimidin-2-yl)amino)cyclohexypacetamide [3242];
1-((1 r,40-4-45-(imidazo [1 ,2-b]pyridazin-6-yl)-4-(methylamino)-7H-pyrrolo [2,3-d]pyrimidin-2-yl)amino)cyclohexyl)pyrrolidin-2-one [3243];
1-((1 s,4s)-4-((5-(imidazo [1 ,2-b]pyridazin-6-yl)-4-(methylamino)-7H-pyrrolo [2,3-d]pyrimidin-2-yl)amino)cyclohexyl)pyrrolidin-2-one [3244];
5-(imidazo [1 ,2-b]pyridazin-6-yl)-N²-((1 s,4s)-4-methoxycyclohexyl)-N⁴-methyl -7H-pyrrolo [2,3-d]pyrimidine-2,4-diamine [3245];
N²-4 1 s,4s)-4-(difluoromethoxy)cyclohexyl)-5-(imidazo[1 ,2-b]pyridazin-6-yl)-N⁴-methyl -7H-pyrrolo [2,3-d]pyrimidine-2,4-diamine [3246];
N²-((1r,4r) -4-(difluoromethoxy)cyclohexyl)-5-(imidazo [1,2-b] pyridazin-6-yl)-N⁴-methyl-7H-pyrrolo [2,3-d]pyrimidine-2,4-diamine [3247];
N-((1s,4s)-4-((5-(imidazo [1,2-b]pyridazin-6-yl)-4-(methylamino)-7H-pyrrolo [2,3-d]pyrimidin-2-yl)amino)-1-methylcyclohexyl)acetamide [3248];
N-((1r,40-4-45-(imidazo [1,2-b]pyridazin-6-yl)-4-(methylamino)-7H-pyrrolo [2,3-d]pyrimidin-2-yl)amino)-1-methylcyclohexyl)acetamide [3249];
1-((1s,4s)-4-((5-(imidazo [1,2-b]pyridazin-6-yl)-4-(methylamino)-7H-pyrrolo [2,3-d]pyrimidin-2-yl)amino)-1-methylcyclohexyl)pyrrolidin-2-one [3250];
1-((1r,40-4-45-(imidazo [1,2-b]pyridazin-6-yl)-4-(methylamino)-7H-pyrrolo [2,3-d] pyrimidin-2-yl)amino)-1-methylcyclohexyl)pyrrolidin-2-one [3251];
(1s,4s)-4-((5-(imidazo [1,2-b] pyridazin-6-yl)-4-(methylamino)-7H-pyrrolo [2,3-d]pyrimidin-2-yl)amino)-1-methylcyclohexan-1-ol [3252];
(1r,4r) -4-45-(imidazo [1,2-b] pyridazin-6-yl)-4-(methylamino)-7H-pyrrolo [2,3-d] pyrimidin-2-yl)amino)-1-methylcyclohexan-1-ol [3253];
N²-((3R,4S)-3-fluoro-1-methylpiperidin-4-yl)-5-(imidazo [1,2-b] pyridazin-6-yl)-N⁴-methyl-7H-pyrrolo [2,3-d]pyrimidine-2,4-diamine [3254];
N²-((3 S,4R)-3-fluoro-1-methylpiperidin-4-yl)-5-(imidazo [1,2-b] pyridazin-6-yl)-N⁴-methyl-7H-pyrrolo [2,3-d]pyrimidine-2,4-diamine [3255];
N²-((3 S,4S)-3-fluoro-1-methylpiperidin-4-yl)-5-(imidazo [1,2-b] pyridazin-6-yl)-N⁴-methyl-7H-pyrrolo [2,3-d]pyrimidine-2,4-diamine [3256];
N²-((3R,4R)-3-fluoro-1-methylpiperidin-4-yl)-5-(imidazo [1,2-b]pyridazin-6-yl)-N⁴-methyl-7H-pyrrolo [2,3-d]pyrimidine-2,4-diamine [3257];
1-(7-((5-(imidazo [1,2-b] pyridazin-6-yl)-4-(methylamino)-7H-pyrrolo [2,3-d]pyrimidin-2-yl)amino)-2-azaspiro [3 .5] nonan-2-yl)ethan-1-one [3258];
5-(imidazo [1,2-b]pyridazin-6-yl)-N⁴-methyl-N²-(2-oxaspiro [3 .5]nonan-7-yl)-7H-pyrrolo [2,3-d]pyrimidine -2,4-diamine [3259];
(1s,3s)-N,N,1-trimethyl-3-45-(pyrazolo [1,5-a]pyridin-5-yl)-7H-pyrrolo [2,3-d] pyrimidin-2-yl)amino)cyclobutane-1-carboxamide [3260];
(1r,30-N,N,1-trimethyl-3-45-(pyrazolo [1,5-a]pyridin-5-yl)-7H-pyrrolo [2,3-d] pyrimidin-2-yl)amino)cyclobutane-1-carboxamide [3261];
((1s,3s)-1-methyl-3-45-(pyrazolo [1,5-a]pyridin-5-yl)-7H-pyrrolo [2,3-d]pyrimidin-2-yl)amino)cyclobutyl)(pyrrolidin-1-yl)methanone [3262];
((1r,3r)-1-methyl-3-45-(pyrazolo [1,5-a] pyridin-5-yl)-7H-pyrrolo [2,3-d]pyrimidin-2-yl)amino)cyclobutyl)(pyrrolidin-1-yl)methanone [3263];
N-((1s,3s)-1-methyl-3-45-(pyrazolo [1,5-a]pyridin-5-yl)-7H-pyrrolo[2,3-d]pyrimidin-2-yl)amino)cyclobutypacetamide [3264];
N-((1r,3r)-1-methyl-3-45-(pyrazolo [1,5-a]pyridin-5-yl)-7H-pyrrolo[2,3-d]pyrimidin-2-yl)amino)cyclobutypacetamide [3265];
N-((1s,3s)-1-methyl-3-45-(pyrazolo [1,5-a]pyridin-5-yl)-7H-pyrrolo[2,3-d]pyrimidin-2-yl)amino)cyclobutyl)propionamide [3266];
N-((1r,3r)-1-methyl-3-45-(pyrazolo [1,5-a]pyridin-5-yl)-7H-pyrrolo[2,3-d]pyrimidin-2-yl)amino)cyclobutyl)propionamide [3267];
1-((1r,3r)-1-methyl-3-45-(pyrazolo [1,5-a]pyridin-5-yl)-7H-pyrrolo [2,3-d] pyrimidin-2-yl)amino)cyclobutyl)pyrrolidin-2-one [3268];
1-((1s,3s)-1-methyl-3-45-(pyrazolo [1,5-a]pyridin-5-yl)-7H-pyrrolo [2,3-d] pyrimidin-2-yl)amino)cyclobutyl)pyrrolidin-2-one [3269];
(1s,3s)-1-methyl-3-45-(pyrazolo [1,5-a] pyridin-5-yl)-7H-pyrrolo [2,3-d]pyrimidin-2-yl)amino)cyclobutan-1-ol [3270];
(1r,3r)-1-methyl-3-45-(pyrazolo [1,5-a]pyridin-5-yl)-7H-pyrrolo [2,3-d] pyrimidin-2-yl)amino)cyclobutan-1-ol [3271];
(1s,4s)-N,N-dimethyl-4-45-(pyrazolo [1,5-a]pyridin-5-yl)-7H-pyrrolo [2,3-d]pyrimidin-2-yl)amino)cyclohexane-1-carboxamide [3272];
((1s,4s)-4-((5-(pyrazolo [1,5-a]pyridin-5-yl)-7H-pyrrolo [2,3-d] pyrimidin-2-yl)amino)cyclohexyl)(pyrrolidin-1-yl)methanone [3273];
N-((1s,4s)-4-45-(pyrazolo [1,5-a]pyridin-5-yl)-7H-pyrrolo [2,3-d] pyrimidin-2-yl)amino)cyclohexyl)acetamide [3274];
N-((1r,4r)-4-((5-(pyrazolo [1,5-a]pyridin-5-yl)-7H-pyrrolo [2,3-d] pyrimidin-2-yl)amino)cyclohexyl)acetamide [3275];
1-((1s,4s)-4-45-(pyrazolo [1,5-a]pyridin-5-yl)-7H-pyrrolo [2,3-d] pyrimidin-2-yl)amino)cyclohexyl)pyrrolidin-2-one [3276];
N-((1r,40-4-methoxycyclohexyl)-5-(pyrazolo [1,5-a]pyridin-5-yl)-7H-pyrrolo [2,3-d]pyrimidin-2-amine [3277];
N-((1r,4r)-4-(difluoromethoxy)cyclohexyl)-5-(pyrazolo [1,5-a]pyridin-5-yl)-7H-pyrrolo [2,3-d]pyrimidin-2-amine [3278];
2-(((1r,4r)-4-((5-(pyrazolo [1,5-a]pyridin-5-yl)-7H-pyrrolo [2,3-d] pyrimidin-2-yl)amino)cyclohexyl)oxy)ethan-1-ol [3279];
N-((1r,4r)-1-methyl-4-45-(pyrazolo [1,5-a]pyridin-5-yl)-7H-pyrrolo[2,3-d]pyrimidin-2-yl)amino)cyclohexypacetamide [3280];
(1s,4s)-1-ethyl-4-((5-(pyrazolo [1,5-a]pyridin-5-yl)-7H-pyrrolo [2,3-d]pyrimidin-2-yl)amino)cyclohexan-1-01 [3281];
1-(4-((5-(pyrazolo [1,5-a] pyridin-5-yl)-7H-pyrrolo [2,3-d] pyrimidin-2-yl)amino)piperidin-1-yl)ethan-1-one [3282];
(S)-1-methyl-5-45-(pyrazolo [1,5-a]pyridin-5-yl)-7H-pyrrolo [2,3-d]pyrimidin-2-yl)amino)piperidin-2-one [3283];
(R)-1-methyl-5-45-(pyrazolo [1,5-a]pyridin-5-yl)-7H-pyrrolo [2,3-d] pyrimidin-2-yl)amino)piperidin-2-one [3284];
1-(2-45-(pyrazolo [1,5-a] pyridin-5-yl)-7H-pyrrolo [2,3-d] pyrimidin-2-yl)amino)-7-azaspiro [3 .5]nonan-7-yl)ethan-1-one [3285];
1-((3aR,5s,6aS)-5-45-(pyrazolo [1,5-a] pyridin-5-yl)-7H-pyrrolo [2,3-d] pyrimidin-2-yl)amino)hexahydrocyclopenta[c]pyrrol-2(1H)-yl)ethan-1-one [3286];
1-((3aR,5r,6aS)-5-((5-(pyrazolo [1,5-a]pyridin-5-yl)-7H-pyrrolo [2,3-d]pyrimidin-2-yl)amino)hexahydrocyclopenta[c]pyrrol-2(1H)-yl)ethan-1-one [3287];
1-(7-45-(pyrazolo [1,5-a] pyridin-5-yl)-7H-pyrrolo [2,3-d] pyrimidin-2-yl)amino)-2-azaspiro [3 .5]nonan-2-yl)ethan-1-one [3288];
5-(pyrazolo [1,5-a]pyridin-5-yl)-N-(2-oxaspiro [3 .5]nonan-7-yl)-7H-pyrrolo [2,3-d]pyrimidin-2-amine [3289];
5-(pyrazolo [1,5-a]pyridin-5-yl)-N-(1,4-dioxaspiro [4.5] de can-8-yl)-7H-pyrrolo [2,3-d] pyrimidin-2-amine [3290];
4-methoxy-5-(pyrazolo [1,5-a]pyridin-5-yl)-7H-pyrrolo [2,3-d]pyrimidin-2-amine [3291];
(1r,30-3-44-methoxy-5-(pyrazolo [1,5-a]pyridin-5-yl)-7H-pyrrolo [2,3-d]pyrimidin-2-yl)amino)-N,N,1-trimethylcyclobutane-1-carboxamide [3292];
(1s,3s)-3-44-methoxy-5-(pyrazolo [1,5-a] pyridin-5-yl)-7H-pyrrolo [2,3-d] pyrimidin-2-yl)amino)-N,N,1-trimethylcyclobutane-1-carboxamide [3293];
((1s,3s)-3-((4-methoxy-5-(pyrazolo [1,5-a]pyridin-5-yl)-7H-pyrrolo [2,3-d]pyrimidin-2-yl)amino)-1-methylcyclobutyl)(pyrrolidin-1-yl)methanone [3294];
((1r,30-3-44-methoxy-5-(pyrazolo [1,5-a] pyridin-5-yl)-7H-pyrrolo [2,3-d]pyrimidin-2-yl)amino)-1-methylcyclobutyl)(pyrrolidin-1-yl)methanone [3295];
N-((1s,3s)-3-((4-methoxy-5-(pyrazolo [1,5-a]pyridin-5-yl)-7H-pyrrolo [2,3-d] pyrimidin-2-yl)amino)-1-methylcyclobutypacetamide [3296];
N-((1r,3r)-3-((4-methoxy-5-(pyrazolo [1,5-a]pyridin-5-yl)-7H-pyrrolo [2,3-d] pyrimidin-2-yl)amino)-1-methylcyclobutypacetamide [3297];
N-((1 s,3 s)-3-((4-methoxy-5-(pyrazolo [1,5-a]pyridin-5-yl)-7H-pyrrolo [2,3-d]pyrimidin-2-yl)amino)-1-methylcyclobutyppropionamide [3298];
N-((1 r,3 r)-3-((4-methoxy-5-(pyrazolo [1,5-a]pyridin-5-yl)-7H-pyrrolo [2,3-d]pyrimidin-2-yl)amino)-1-methylcyclobutyppropionamide [3299];
1-((1 r,3 r)-3-((4-methoxy-5-(pyrazolo [1,5-a]pyridin-5-yl)-7H-pyrrolo [2,3-d]pyrimidin-2-yl)amino)-1-methylcyclobutyppyrrolidin-2-one [3300];
1-((1 s,3 s)-3-((4-methoxy-5-(pyrazolo [1,5-a]pyridin-5-yl)-7H-pyrrolo [2,3-d]pyrimidin-2-yl)amino)-1-methylcyclobutyppyrrolidin-2-one [3301];
(1 s,3 s)-3-((4-methoxy-5-(pyrazolo [1,5-a] pyridin-5-yl)-7H-pyrrolo [2,3-d]pyrimidin-2-yl)amino)-1-methylcyclobutan-1-ol [3302];
(1 r,3 r)-3-((4-methoxy-5-(pyrazolo [1,5-a]pyridin-5-yl)-7H-pyrrolo [2,3-d]pyrimidin-2-yl)amino)-1-methylcyclobutan-1-ol [3303];
(1 s,4s)-4-((4-methoxy-5-(pyrazolo [1,5-a] pyridin-5-yl)-7H-pyrrolo [2,3-d]pyrimidin-2-yl)amino)-N,N-dimethylcyclohexane-1-carboxamide [3304];
((1 s,4 s)-4-((4-methoxy -5-(pyrazolo [1,5-a]pyridin-5-yl)-7H-pyrrolo [2,3-d]pyrimidin-2-yl)amino)cyclohexyl)(pyrrolidin-1-yl)methanone [3305];
N-((1 s,4s)-4-((4-methoxy-5-(pyrazolo [1,5-a]pyridin-5-yl)-7H-pyrrolo [2,3-d]pyrimidin-2-yl)amino)cyclohexypacetamide [3306];
N-((1 r,4r)-4-((4-methoxy-5-(pyrazolo [1,5-a]pyridin-5-yl)-7H-pyrrolo [2,3-d]pyrimidin-2-yl)amino)cyclohexypacetamide [3307];
1-((1 s,4s)-4-((4-methoxy-5-(pyrazolo [1,5-a]pyridin-5-yl)-7H-pyrrolo [2,3-d]pyrimidin-2-yl)amino)cyclohexyl)pyrrolidin-2-one [3308];
5-([1,2,4]triazolo [1,5-a]pyridin-7-yl)-4-methoxy-N-((1 s,4s)-4-methoxycyclohexyl)-7H-pyrrolo [2,3-d]pyrimidin-2-amine [3309];
4-methoxy-N-((1 r,40-4-methoxycyclohexyl)-5-(pyrazolo [1,5-a]pyridin-5-yl)-7H-pyrrolo [2,3-d]pyrimidin-2-amine [3310];
4-(methoxy-d₃)-N-((1 s,4s)-4-methoxycyclohexyl)-5-(pyrazolo [1,5-a]pyridin-5-yl)-7H-pyrrolo [2,3-d]pyrimidin-2-amine [3311];
N-((1 r,4r)-4-((4-methoxy-5-(pyrazolo [1,5-a]pyridin-5-yl)-7H-pyrrolo [2,3-d]pyrimidin-2-yl)amino)-1-methylcyclohexyl)acetamide [3312];
(1 r,4r) -4-44-(methoxy-d₃)-5-(pyrazolo [1,5-a]pyridin-5-yl)-7H-pyrrolo [2,3-d]pyrimidin-2-yl)amino)-1-methylcyclohexan-1-ol [3313];
(1 s,4s)-4-44-(methoxy-d₃)-5-(pyrazolo [1,5-a]pyridin-5-yl)-7H-pyrrolo [2,3-d]pyrimidin-2-yl)amino)-1-methylcyclohexan-1-ol [3314];
(1s,4s)-1-ethyl-4-44-methoxy-5-(pyrazolo [1,5-a]pyridin-5-yl)-7H-pyrrolo [2,3-d]pyrimidin-2-yl)amino)cyclohexan-1-01 [3315];
1-(4-((4-methoxy-5-(pyrazolo [1,5-a]pyridin-5-yl)-7H-pyrrolo [2,3-d]pyrimidin-2-yl)amino)piperidin-1-yl)ethan-1-one [3316];
(S)-5-((4-methoxy-5-(pyrazolo [1,5-a]pyridin-5-yl)-7H-pyrrolo [2,3-d]pyrimidin-2-yl)amino)-1-methylpiperidin-2-one [3317];
(R)-5-((4-methoxy-5-(pyrazolo [1,5-a]pyridin-5-yl)-7H-pyrrolo [2,3-d]pyrimidin-2-yl)amino)-1-methylpiperidin-2-one [3318];
N-((1R,5S,6r)-3-oxabicyclo [3.1.0]hexan-6-yl)-4-methoxy-5-(pyrazolo [1,5-a]pyridin-5-yl)-7H-pyrrolo [2,3-d]pyrimidin-2-amine [3319];
N-((1R,5S,6s)-3-oxabicyclo [3.1.0]hexan-6-yl)-4-methoxy-5-(pyrazolo [1,5-a]pyridin-5-yl)-7H-pyrrolo [2,3-d]pyrimidin-2-amine [3320];
4-methoxy-5-(pyrazolo [1,5-a]pyridin-5-yl)-N-(2-oxaspiro [3.3]heptan-6-yl)-7H-pyrrolo [2,3-d]pyrimidin-2-amine [3321];
1-(2-((4-methoxy-5-(pyrazolo [1,5-a]pyridin-5-yl)-7H-pyrrolo [2,3-d]pyrimidin-2-yl)amino)-7-azaspiro [3 .5]nonan-7-yl)ethan-1-one [3322];
1-((3aR,5s,6aS)-5-((4-methoxy-5-(pyrazolo [1,5-a]pyridin-5-yl)-7H-pyrrolo [2,3-d]pyrimidin-2-yl)amino)hexahydrocyclopenta[c]pyrrol-2(1H)-yl)ethan-1-one [3323];
1-((3aR,5r,6aS)-5-((4-methoxy-5-(pyrazolo [1,5-a]pyridin-5-yl)-7H-pyrrolo [2,3-d]pyrimidin-2-yl)amino)hexahydrocyclopenta[c]pyrrol-2(1H)-yl)ethan-1-one [3324];
1-(7-((4-methoxy-5-(pyrazolo [1,5-a]pyridin-5-yl)-7H-pyrrolo [2,3-d]pyrimidin-2-yl)amino)-2-azaspiro [3 .5]nonan-2-yl)ethan-1-one [3325];
4-methoxy-5-(pyrazolo [1,5-a]pyridin-5-yl)-N-(2-oxaspiro [3.5]nonan-7-yl)-7H-pyrrolo [2,3-d]pyrimidin-2-amine [3326];
5-(4-methoxy-2-(((1s,4s)-4-methoxycyclohexyl)amino)-7H-pyrrolo [2,3-d]pyrimidin-5-yl)-N-methylpyrazolo [1,5-a]pyridine-3-carboxamide [3327];
5-(2-(((1s,4s)-4-hydroxy-4-methylcyclohexyl)amino)-4-methoxy-7H-pyrrolo [2,3-d]pyrimidin-5-yl)-N-methylpyrazolo [1,5-a] pyridine-3-carboxamide [3328];
5-(2-(((1r,4r)-4-hydroxy-4-methylcyclohexyl)amino)-4-methoxy-7H-pyrrolo [2,3-d]pyrimidin-5-yl)-N-methylpyrazolo [1,5-a] pyridine-3-carboxamide [3329];
5-(2-(((1r,4r) -4-hydroxy-4-methylcyclohexyl)amino)-4-(methoxy-d₃)-7H-pyrrolo [2,3-d]pyrimidin-5-yl)-N-methylpyrazolo [1,5-a]pyridine-3-carboxamide [3330];
5-(2-(((1s,4s)-4-hydroxy-4-methylcyclohexyl)amino)-4-(methoxy-d₃)-7H-pyrrolo [2,3-d]pyrimidin-5-yl)-N-methylpyrazolo [1,5-a]pyridine-3-carboxamide [3331];
5-(2-(((1s,4s)-4-ethyl-4-hydroxycyclohexyl)amino)-4-methoxy-7H-pyrrolo [2,3-d] pyrimidin-5-yl)-N-methylpyrazolo [1,5-a] pyridine-3-carboxamide [3332];
5-(2-((2-oxaspiro [3.5]nonan-7-yl)amino)-4-methoxy-7H-pyrrolo [2,3-d]pyrimidin-5-yl)-N-methylpyrazolo [1,5-a]pyridine-3-carboxamide [3333];
(1s,3s)-3-45-([1,2,4]triazolo [1,5-a]pyridin-7-yl)-7H-pyrrolo [2,3-d]pyrimidin-2-yl)amino)-N,N,1-trimethylcyclobutane-1-carboxamide [3334];
(1r,3r)-3-((5-([1,2,4]triazolo [1,5-a]pyridin-7-yl)-7H-pyrrolo [2,3-d]pyrimidin-2-yl)amino)-N,N,1-trimethylcyclobutane-1-carboxamide [3335];
((1s,3s)-3-((5-([1,2,4]triazolo [1,5-a]pyridin-7-yl)-7H-pyrrolo [2,3-d] pyrimidin-2-yl)amino)-1-methylcyclobutyl)(pyrrolidin-1-yl)methanone [3336];
((1r,3r)-3-((5-([1,2,4]triazolo [1,5-a]pyridin-7-yl)-7H-pyrrolo [2,3-d]pyrimidin-2-yl)amino)-1-methylcyclobutyl)(pyrrolidin-1-yl)methanone [3337];
N-((1s,3s)-3-((5-([1,2,4]triazolo [1,5-a]pyridin-7-yl)-7H-pyrrolo [2,3-d]pyrimidin-2-yl)amino)-1-methylcyclobutypacetamide [3338];
N-((1r,3r)-3-((5-([1,2,4]triazolo [1,5-a]pyridin-7-yl)-7H-pyrrolo [2,3-d]pyrimidin-2-yl)amino)-1-methylcyclobutypacetamide [3339];
N-((1s,3s)-3-((5-([1,2,4]triazolo [1,5-a]pyridin-7-yl)-7H-pyrrolo [2,3-d]pyrimidin-2-yl)amino)-1-methylcyclobutyl)propionamide [3340];
N-((1r,3r)-3-((5-([1,2,4]triazolo [1,5-a]pyridin-7-yl)-7H-pyrrolo [2,3-d]pyrimidin-2-yl)amino)-1-methylcyclobutyl)propionamide [3341];
1-((1r,3r)-3-((5-([1,2,4]triazolo [1,5-a]pyridin-7-yl)-7H-pyrrolo [2,3-d]pyrimidin-2-yl)amino)-1-methylcyclobutyl)pyrrolidin-2-one [3342];
1-((1s,3s)-3-((5-([1,2,4]triazolo [1,5-a]pyridin-7-yl)-7H-pyrrolo [2,3-d]pyrimidin-2-yl)amino)-1-methylcyclobutyl)pyrrolidin-2-one [3343];
(1s,3s)-3-45-([1,2,4]triazolo [1,5-a]pyridin-7-yl)-7H-pyrrolo [2,3-d]pyrimidin-2-yl)amino)-1-methylcyclobutan-1-ol [3344];
(1r,3r)-3-((5-([1,2,4]triazolo [1,5-a] pyridin-7-yl)-7H-pyrrolo [2,3-d]pyrimidin-2-yl)amino)-1-methylcyclobutan-1-ol [3345];
(1s,4s)-4-45-([1,2,4]triazolo [1,5-a]pyridin-7-yl)-7H-pyrrolo [2,3-d]pyrimidin-2-yl)amino)-N,N-dimethylcyclohexane-1-carboxamide [3346];
((1s,4s)-4-((5-([1,2,4]triazolo [1,5-a]pyridin-7-yl)-7H-pyrrolo [2,3-d] pyrimidin-2-yl)amino)cyclohexyl)(pyrrolidin-1-yl)methanone [3347];
N-((1s,4s)-4-45-([1,2,4]triazolo [1,5-a]pyridin-7-yl)-7H-pyrrolo [2,3-d]pyrimidin-2-yl)amino)cyclohexypacetamide [3348];
N-((1r,4r)-4-((5-([1,2,4]triazolo [1,5-a]pyridin-7-yl)-7H-pyrrolo [2,3-d]pyrimidin-2-yl)amino)cyclohexypacetamide [3349];
1-((1s,4s)-4-45-([1,2,4]triazolo [1,5-a]pyridin-7-yl)-7H-pyrrolo [2,3-d]pyrimidin-2-yl)amino)cyclohexyl)pyrrolidin-2-one [3350];
5-([1,2,4]triazolo [1,5-a]pyridin-7-yl)-N-((1s,4s)-4-methoxycyclohexyl)-7H-pyrrolo [2,3-d]pyrimidin-2-amine [3351];
2-(((1s,4s)-4-45-([1,2,4]triazolo [1,5-a] pyridin-7-yl)-7H-pyrrolo [2,3-d] pyrimidin-2-yl)amino)cyclohexyl)oxy)ethan-1-ol [3352];
2-(((1s,4s)-4-45-([1,2,4]triazolo [1,5-a] pyridin-7-yl)-7H-pyrrolo [2,3-d] pyrimidin-2-yl)amino)cyclohexyl)oxy)ethan-1-ol [3353];
N-((1r,40-4-45-([1,2,4]triazolo [1,5-a]pyridin-7-yl)-7H-pyrrolo [2,3-d]pyrimidin-2-yl)amino)-1-methylcyclohexyl)acetamide [3354];
(1r,4r)-4-((5-([1,2,4]triazolo [1,5-a] pyridin-7-yl)-7H-pyrrolo [2,3-d]pyrimidin-2-yl)amino)-1-methylcyclohexan-1-ol [3355];
(1s,4s)-4-45-([1,2,4]triazolo [1,5-a]pyridin-7-yl)-7H-pyrrolo [2,3-d]pyrimidin-2-yl)amino)-1-methylcyclohexan-1-ol [3356];
(1s,4s)-4-45-([1,2,4]triazolo [1,5-a]pyridin-7-yl)-7H-pyrrolo [2,3-d]pyrimidin-2-yl)amino)-1-ethylcyclohexan-1-ol [3357];
1-(4-45-([1,2,4]triazolo [1,5-a]pyridin-7-yl)-7H-pyrrolo [2,3-d]pyrimidin-2-yl)amino)piperidin-1-yl)ethan-1-one [3358];
(R)-5-45-([1,2,4]triazolo [1,5-a]pyridin-7-yl)-7H-pyrrolo [2,3-d]pyrimidin-2-yl)amino)-1-methylpiperidin-2-one [3359];
(S)-5-((5-([1,2,4]triazolo [1,5-a]pyridin-7-yl)-7H-pyrrolo [2,3-d] pyrimidin-2-yl)amino)-1-methylpiperidin-2-one [3360];
1-(2-45-([1,2,4]triazolo [1,5-a]pyridin-7-yl)-7H-pyrrolo [2,3-d]pyrimidin-2-yl)amino)-7-azaspiro [3 .5]nonan-7-yl)ethan-1-one [3361];
1-((3aR,5s,6aS)-5-45-([1,2,4]triazolo [1,5-a]pyridin-7-yl)-7H-pyrrolo [2,3-d]pyrimidin-2-yl)amino)hexahydrocyclopenta[c]pyrrol-2(1H)-yl)ethan-1-one [3362];
1-((3aR,5r,6aS)-5-((5-([1,2,4]triazolo [1,5-a]pyridin-7-yl)-7H-pyrrolo [2,3-d]pyrimidin-2-yl)amino)hexahydrocyclopenta[c]pyrrol-2(1H)-yl)ethan-1-one [3363];
1-(7-45-([1,2,4]triazolo [1,5-a]pyridin-7-yl)-7H-pyrrolo [2,3-d]pyrimidin-2-yl)amino)-2-azaspiro [3 .5]nonan-2-yl)ethan-1-one [3364];
5-([1,2,4]triazolo [1,5-a]pyridin-7-yl)-N-(2-oxaspiro [3 .5] nonan-7-yl)-7H-pyrrolo [2,3-d]pyrimidin-2-amine [3365];
1-([1,2,4]triazolo [1,5-a]pyridin-7-yl)-N-(1,4-dioxaspiro [4.5] decan-8-yl)-7H-pyrrolo [2,3-d]pyrimidin-2-amine [3366];
(1s,3s)-3-((5-([1,2,4]triazolo [1,5-a]pyridin-7-yl)-4-methoxy-7H-pyrrolo [2,3-d]pyrimidin-2-yl)amino)-N,N,1-trimethylcyclobutane-1-carboxamide [3367];
(1r,3r)-3-((5-([1,2,4]triazolo [1,5-a] pyridin-7-yl)-4-methoxy-7H-pyrrolo [2,3-d] pyrimidin-2-yl)amino)-N,N,1-trimethylcyclobutane-1-carboxamide [3368];
((1r,3r)-3-((5-([1,2,4]triazolo [1,5-a]pyridin-7-yl)-4-methoxy-7H-pyrrolo [2,3-d]pyrimidin-2-yl)amino)-1-methylcyclobutyl)(pyrrolidin-1-yl)methanone [3369];
((1s,3s)-3-((5-([1,2,4]triazolo [1,5-a]pyridin-7-yl)-4-methoxy-7H-pyrrolo [2,3-d]pyrimidin-2-yl)amino)-1-methylcyclobutyl)(pyrrolidin-1-yl)methanone [3370];
N-((1s,3s)-3-((5-([1,2,4]triazolo [1,5-a]pyridin-7-yl)-4-methoxy-7H-pyrrolo [2,3-d] pyrimidin-2-yl)amino)-1-methylcyclobutypacetamide [3371];
N-((1r,3r)-3-((5-([1,2,4]triazolo [1,5-a]pyridin-7-yl)-4-methoxy-7H-pyrrolo [2,3-d] pyrimidin-2-yl)amino)-1-methylcyclobutypacetamide [3372];
N-((1s,3s)-3-((5-([1,2,4]triazolo [1,5-a]pyridin-7-yl)-4-methoxy-7H-pyrrolo [2,3-d] pyrimidin-2-yl)amino)-1-methylcyclobutyl)propionamide [3373];
N-((1r,3r)-3-((5-([1,2,4]triazolo [1,5-a]pyridin-7-yl)-4-methoxy-7H-pyrrolo [2,3-d] pyrimidin-2-yl)amino)-1-methylcyclobutyl)propionamide [3374];
1-((1r,3r)-3-((5-([1,2,4]triazolo [1,5-a]pyridin-7-yl)-4-methoxy-7H-pyrrolo [2,3-d]pyrimidin-2-yl)amino)-1-methylcyclobutyppyrrolidin-2-one [3375];
1-((1s,3s)-3-((5-([1,2,4]triazolo [1,5-a]pyridin-7-yl)-4-methoxy-7H-pyrrolo [2,3-d] pyrimidin-2-yl)amino)-1-methylcyclobutyl)pyrrolidin-2-one [3376];
(1s,3s)-3-((5-([1,2,4]triazolo [1,5-a]pyridin-7-yl)-4-methoxy-7H-pyrrolo [2,3-d]pyrimidin-2-yl)amino)-1-methylcyclobutan-1-ol [3377];
(1r,3r)-3-((5-([1,2,4]triazolo [1,5-a] pyridin-7-yl)-4-methoxy-7H-pyrrolo [2,3-d] pyrimidin-2-yl)amino)-1-methylcyclobutan-1-ol [3378];
(1s,4s)-4-((5-([1,2,4]triazolo [1,5-a]pyridin-7-yl)-4-methoxy-7H-pyrrolo [2,3-d]pyrimidin-2-yl)amino)-N,N-dimethylcyclohexane-1-carboxamide [3379];
((1s,4s)-4-((5-([1,2,4]triazolo [1,5-a]pyridin-7-yl)-4-methoxy-7H-pyrrolo [2,3-d]pyrimidin-2-yl)amino)cyclohexyl)(pyrrolidin-1-yl)methanone [3380];
N-((1s,4s)-4-((5-([1,2,4]triazolo [1,5-a]pyridin-7-yl)-4-methoxy-7H-pyrrolo [2,3-d] pyrimidin-2-yl)amino)cyclohexyl)acetamide [3381];
N-((1r,4r)-4-((5-([1,2,4]triazolo [1,5-a]pyridin-7-yl)-4-methoxy-7H-pyrrolo [2,3-d] pyrimidin-2-yl)amino)cyclohexyl)acetamide [3382];
1-((1s,4s)-4-((5-([1,2,4]triazolo [1,5-a]pyridin-7-yl)-4-methoxy-7H-pyrrolo [2,3-d] pyrimidin-2-yl)amino)cyclohexyl)pyrrolidin-2-one [3383];
5-([1,2,4]triazolo [1,5-a]pyridin-7-yl)-4-methoxy-N-((1s,4s)-4-methoxycyclohexyl)-7H-pyrrolo [2,3-d]pyrimidin-2-amine [3384];
5-([1,2,4]triazolo [1,5-a]pyridin-7-yl)-4-(methoxy-d₃)-N-((1s,4s)-4-methoxycyclohexyl)-7H-pyrrolo [2,3-d]pyrimidin-2-amine [3385];
2-(((1s,4s)-4-((5-([1,2,4]triazolo [1,5-a] pyridin-7-yl)-4-methoxy-7H-pyrrolo [2,3-d] pyrimidin-2-yl)amino)cyclohexyl)oxy)ethan-1-ol [3386];
2-(((1r,4r)-4-((5-([1,2,4]triazolo [1,5-a]pyridin-7-yl)-4-methoxy-7H-pyrrolo [2,3-d] pyrimidin-2-yl)amino)cyclohexyl)oxy)ethan-1-ol [3387];
N-((1r,4r)-4-((5-([1,2,4]triazolo [1,5-a]pyridin-7-yl)-4-methoxy-7H-pyrrolo [2,3-d] pyrimidin-2-yl)amino)-1-methylcyclohexyl)acetamide [3388];
(1r,4r)-4-((5-([1,2,4]triazolo [1,5-a]pyridin-7-yl)-4-methoxy-7H-pyrrolo [2,3-d] pyrimidin-2-yl)amino)-1-methylcyclohexan-1-ol [3389];
(1s,4s)-4-((5-([1,2,4]triazolo [1,5-a]pyridin-7-yl)-4-methoxy-7H-pyrrolo [2,3-d]pyrimidin-2-yl)amino)-1-methylcyclohexan-1-ol [3390];
(1r,4r)-4-((5-([1,2,4]triazolo [1,5-a]pyridin-7-yl)-4-(methoxy-d₃)-7H-pyrrolo [2,3-d]pyrimidin-2-yl)amino)-1-methylcyclohexan-1-ol [3391];
(1s,4s)-4-((5-([1,2,4]triazolo [1,5-a]pyridin-7-yl)-4-(methoxy-d₃)-7H-pyrrolo [2,3-d]pyrimidin-2-yl)amino)-1-methylcyclohexan-1-ol [3392];
(1s,4s)-4-((5-([1,2,4]triazolo [1,5-a]pyridin-7-yl)-4-methoxy-7H-pyrrolo [2,3-d]pyrimidin-2-yl)amino)-1-ethylcyclohexan-1-ol [3393];
1-(4-((5-([1,2,4]triazolo [1,5-a]pyridin-7-yl)-4-methoxy-7H-pyrrolo [2,3-d]pyrimidin-2-yl)amino)piperidin-1-yl)ethan-1-one [3394];
(S)-5-((5-([1,2,4]triazolo [1,5-a]pyridin-7-yl)-4-methoxy-7H-pyrrolo [2,3-d] pyrimidin-2-yl)amino)-1-methylpiperidin-2-one [3395];
(R)-5-((5-([1,2,4]triazolo [1,5-a]pyridin-7-yl)-4-methoxy-7H-pyrrolo [2,3-d]pyrimidin-2-yl)amino)-1-methylpiperidin-2-one [3396];
1-(2-((5-([1,2,4]triazolo [1,5-a]pyridin-7-yl)-4-methoxy-7H-pyrrolo [2,3-d]pyrimidin-2-yl)amino)-7-azaspiro [3 .5] nonan-7-yl)ethan-1-one [3397];
1-((3aR,5s,6aS)-5-((5-([1,2,4]triazolo [1,5-a]pyridin-7-yl)-4-methoxy-7H-pyrrolo [2,3-d]pyrimidin-2-yl)amino)hexahydrocyclopenta[c] pyrrol-2(1H)-yl)ethan-1-one [3398];
1-((3aR,5r,6aS)-5-((5-([1,2,4]triazolo [1,5-a]pyridin-7-yl)-4-methoxy-7H-pyrrolo [2,3-d]pyrimidin-2-yl)amino)hexahydrocyclopenta [c]pyrrol-2(1H)-yl)ethan-1-one [3399];
1-(7-((5-([1,2,4]triazolo [1,5-a]pyridin-7-yl)-4-methoxy-7H-pyrrolo [2,3-d]pyrimidin-2-yl)amino)-2-azaspiro [3 .5] nonan-2-yl)ethan-1-one [3400];
5-([1,2,4]triazolo [1,5-a]pyridin-7-yl)-4-methoxy-N-(2-oxaspiro [3 .5]nonan-7-yl)-7H-pyrrolo [2,3-d]pyrimidin-2-amine [3401];
5-([1,2,4]triazolo [1,5-a]pyridin-7-yl)-4-methoxy-N-(1,4-dioxaspiro [4 .5] de can-8-yl)-7H-pyrrolo [2,3-d]pyrimidin-2-amine [3402];
(1s,3 s)-N,N,1-trimethyl-3-((5-(pyrazolo [1,5-a]pyrimidin-5-yl)-7H-pyrrolo [2,3-d]pyrimidin-2-yl)amino)cyclobutane-1-carboxamide [3403];
(1r,30-N,N,1-trimethyl-3-((5-(pyrazolo [1,5-a]pyrimidin-5-yl)-7H-pyrrolo [2,3-d]pyrimidin-2-yl)amino)cyclobutane-1-carboxamide [3404];
((1r,3r)-1-methyl-3-((5-(pyrazolo [1,5-a] pyrimidin-5-yl)-7H-pyrrolo [2,3-d] pyrimidin-2-yl)amino)cyclobutyl)(pyrrolidin-1-yl)methanone [3405];
((1s,3s)-1-methyl-3-((5-(pyrazolo [1,5-a]pyrimidin-5-yl)-7H-pyrrolo [2,3-d]pyrimidin-2-yl)amino)cyclobutyl)(pyrrolidin-1-yl)methanone [3406];
N-((1r,3r)-1-methyl-3-((5-(pyrazolo [1,5-a]pyrimidin-5-yl)-7H-pyrrolo [2,3-d]pyrimidin-2-yl)amino)cyclobutypacetamide [3407];
N-((1s,3s)-1-methyl-3-((5-(pyrazolo [1,5-a]pyrimidin-5-yl)-7H-pyrrolo [2,3-d]pyrimidin-2-yl)amino)cyclobutypacetamide [3408];
N-((1s,3s)-1-methyl-3-((5-(pyrazolo [1,5-a]pyrimidin-5-yl)-7H-pyrrolo [2,3-d]pyrimidin-2-yl)amino)cyclobutyl)propionamide [3409];
N-((1r,3r)-1-methyl-3-((5-(pyrazolo [1,5-a]pyrimidin-5-yl)-7H-pyrrolo [2,3-d]pyrimidin-2-yl)amino)cyclobutyl)propionamide [3410];
1-((1r,3r)-1-methyl-3-((5-(pyrazolo [1,5-a]pyrimidin-5-yl)-7H-pyrrolo [2,3-d]pyrimidin-2-yl)amino)cyclobutyl)pyrrolidin-2-one [3411];
1-((1 s,3 s)-1-methyl-3-((5-(pyrazolo [1,5-a]pyrimidin-5-yl)-7H-pyrrolo [2,3-d]pyrimidin-2-yl)amino)cyclobutyl)pyrrolidin-2-one [3412];
(1s,3 s) -1-methyl-3-((5-(pyrazolo [1,5-a] pyrimidin-5-yl)-7H-pyrrolo [2,3-d] pyrimidin-2-yl)amino)cyclobutan-1-01 [3413];
(1r,3r)-1-methyl-3-((5-(pyrazolo [1,5-a]pyrimidin-5-yl)-7H-pyrrolo [2,3-d]pyrimidin-2-yl)amino)cyclobutan-1-01 [3414];
(1s,4s)-N,N-dimethyl-4-((5-(pyrazolo [1,5-a]pyrimidin-5-yl)-7H-pyrrolo [2,3-d] pyrimidin-2-yl)amino)cyclohexane-1-carboxamide [3415];
((1s,4s)-4-((5-(pyrazolo [1,5-a]pyrimidin-5-yl)-7H-pyrrolo [2,3-d]pyrimidin-2-yl)amino)cyclohexyl)(pyrrolidin-1-yl)methanone [3416];
N-((1s,4s)-4-((5-(pyrazolo [1,5-a]pyrimidin-5-yl)-7H-pyrrolo [2,3-c1] pyrimidin-2-yl)amino)cyclohexyl)acetamide [3417];
N-((1r,4r)-4-((5-(pyrazolo [1,5-a]pyrimidin-5-yl)-7H-pyrrolo [2,3-c1] pyrimidin-2-yl)amino)cyclohexyl)acetamide [3418];
1-((1s,4s)-4-((5-(pyrazolo [1,5-a]pyrimidin-5-yl)-7H-pyrrolo [2,3-c1] pyrimidin-2-yl)amino)cyclohexyl)pyrrolidin-2-one [3419];
N-((1r,40-4-methoxycyclohexyl)-5-(pyrazolo [1,5-a]pyrimidin-5-yl)-7H-pyrrolo 2-amine [3420];
N-((1r,40-4-(difluoromethoxy)cyclohexyl)-5-(pyrazolo [1,5-a]pyrimidin-5-yl)-7H-pyrrolo [2,3-cl]pyrimidin-2-amine [3421];
2-(((1r,4r)-4-((5-(pyrazolo [1,5-a]pyrimidin-5-yl)-7H-pyrrolo [2,3-c1] pyrimidin-2-yl)amino)cyclohexyl)oxy)ethan-1-ol [3422];
2-(((1s,4s)-4-((5-(pyrazolo [1,5-a] pyrimidin-5-yl)-7H-pyrrolo [2,3-cl]pyrimidin-2-yl)amino)cyclohexyl)oxy)ethan-1-ol [3423];
N-((1r,4r)-1-methyl-4-45-(pyrazolo [1,5-a]pyrimidin-5-yl)-7H-pyrrolo [2,3-cl]pyrimidin-2-yl)amino)cyclohexypacetamide [3424];
(1r,4r)-1-methyl-4-45-(pyrazolo [1,5-a]pyrimidin-5-yl)-7H-pyrrolo [2,3-cl]pyrimidin-2-yl)amino)cyclohexan-1-01 [3425];
(1s,4s)-1-methyl-4-45-(pyrazolo [1,5-a] pyrimidin-5-yl)-7H-pyrrolo [2,3-cl]pyrimidin-2-yl)amino)cyclohexan-1-01 [3426];
(1s,4s)-1-ethyl-4-45-(pyrazolo [1,5-a]pyrimidin-5-yl)-7H-pyrrolo [2,3-cl]pyrimidin-2-yl)amino)cyclohexan-1-ol [3427];
1-(4-((5-(pyrazolo [1,5-a] pyrimidin-5-yl)-7H-pyrrolo [2,3-cl]pyrimidin-2-yl)amino)piperidin-1-yl)ethan-1-one [3428];
(S)-1-methyl-5-45-(pyrazolo [1,5-a]pyrimidin-5-yl)-7H-pyrrolo [2,3-cl]pyrimidin-2-yl)amino)piperidin-2-one [3429];
(R)-1-methyl-5-45-(pyrazolo [1,5-a]pyrimidin-5-yl)-7H-pyrrolo [2,3-cl]pyrimidin-2-yl)amino)piperidin-2-one [3430];
1-(2-((5-(pyrazolo [1,5-a] pyrimidin-5-yl)-7H-pyrrolo [2,3-cl]pyrimidin-2-yl)amino)-7-azaspiro [3 .5]nonan-7-yl)ethan-1-one [3431];
1-((3aR,5s,6aS)-5-((5-(pyrazolo [1,5-a] pyrimidin-5-yl)-7H-pyrrolo [2,3-cl]pyrimidin-2-yl)amino)hexahydrocyclopenta[c]pyrrol-2(1H)-yl)ethan-1-one [3432];
1-((3aR,5r,6aS)-5-((5-(pyrazolo [1,5-a]pyrimidin-5-yl)-7H-pyrrolo [2,3-cl]pyrimidin-2-yl)amino)hexahydrocyclopenta[c]pyrrol-2(1H)-yl)ethan-1-one [3433];
1-(7-((5-(pyrazolo [1,5-a] pyrimidin-5-yl)-7H-pyrrolo [2,3-d]pyrimidin-2-yl)amino)-2-azaspiro [3 .5]nonan-2-yl)ethan-1-one [3434];
5-(pyrazolo [1,5-a]pyrimidin-5-yl)-N-(2-oxaspiro [3 .5]nonan-7-yl)-7H-pyrrolo [2,3-d]pyrimidin-2-amine [3435];
5-(pyrazolo [1,5-a]pyrimidin-5-yl)-N-(1,4-dioxaspiro [4.5] decan-8-yl)-7H-pyrrolo [2,3-d]pyrimidin-2-amine [3436];
N²-(4,4-difluorocyclohexyl)-N⁴-methyl-5-(pyrazolo [1,5-a]pyrimidin-5-yl)-7H-pyrrolo [2,3-d]pyrimidine -2,4-diamine [3437];
((1s,4s)-4-((4-(methylamino)-5-(pyrazolo [1,5-a]pyrimidin-5-yl)-7H-pyrrolo [2,3-d] pyrimidin-2-yl)amino)cyclohexyl)(pyrrolidin-1-yl)methanone [3438];
((1r,4r)-4-((4-(methylamino)-5-(pyrazolo [1,5-a]pyrimidin-5-yl)-7H-pyrrolo [2,3-d]pyrimidin-2-yl)amino)cyclohexyl)(pyrrolidin-1-yl)methanone [3439];
(1s,4s)-N,N-dimethyl-4-44-(methylamino)-5-(pyrazolo [1,5-a]pyrimidin-5-yl)-7H-pyrrolo [2,3-d]pyrimidin-2-yl)amino)cyclohexane-1-carboxamide [3440];
(1r,40-N,N-dimethyl-4-44-(methylamino)-5-(pyrazolo [1,5-a]pyrimidin-5-yl)-7H-pyrrolo [2,3-d]pyrimidin-2-yl)amino)cyclohexane-1-carboxamide [3441];
N-((1s,4s)-4-((4-(methylamino)-5-(pyrazolo [1,5-a]pyrimidin-5-yl)-7H-pyrrolo [2,3-d] pyrimidin-2-yl)amino)cyclohexyl)acetamide [3442];
N-((1r,40-4-44-(methylamino)-5-(pyrazolo [1,5-a]pyrimidin-5-yl)-7H-pyrrolo [2,3-d] pyrimidin-2-yl)amino)cyclohexyl)acetamide [3443];
1-((1s,4s)-4-((4-(methylamino)-5-(pyrazolo [1,5-a]pyrimidin-5-yl)-7H-pyrrolo [2,3-d] pyrimidin-2-yl)amino)cyclohexyl)pyrrolidin-2-one [3444];
1-((1r,4r)-4-((4-(methylamino)-5-(pyrazolo [1,5-a]pyrimidin-5-yl)-7H-pyrrolo [2,3-d] pyrimidin-2-yl)amino)cyclohexyl)pyrrolidin-2-one [3445];
N²-((1s,4s)-4-methoxycyclohexyl)-N⁴-methyl-5-(pyrazolo [1,5-a]pyrimidin-5-yl)-7H-pyrrolo [2,3-d]pyrimidine-2,4-diamine [3446];
N²-41s,4s)-4-(difluoromethoxy)cyclohexyl)-N⁴-methyl-5-(pyrazolo [1,5-a]pyrimidin-5-yl)-7H-pyrrolo [2,3-d]pyrimidine-2,4-diamine [3447];
N²-41r,4r)-4-(difluoromethoxy)cyclohexyl)-N⁴-methyl-5-(pyrazolo [1,5-a]pyrimidin-5-yl)-7H-pyrrolo [2,3-d]pyrimidine-2,4-diamine [3448];
N-((1s,4s)-1-methyl-4-44-(methylamino)-5-(pyrazolo [1,5-a]pyrimidin-5-yl)-7H-pyrrolo [2,3-d]pyrimidin-2-yl)amino)cyclohexypacetamide [3449];
N-((1r,4r)-1-methyl-4-44-(methylamino)-5-(pyrazolo [1,5-a]pyrimidin-5-yl)-7H-pyrrolo [2,3-d]pyrimidin-2-yl)amino)cyclohexypacetamide [3450];
1-((1 s,4s)-1-methyl-4-((4-(methylamino)-5-(pyrazolo [1,5-a]pyrimidin-5-yl)-7H-pyrrolo [2,3-d]pyrimidin-2-yl)amino)cyclohexyl)pyrrolidin-2-one [3451];
1-((1 r,4r)-1-methyl-4-((4-(methylamino)-5-(pyrazolo [1,5-a]pyrimidin-5-yl)-7H-pyrrolo [2,3-d]pyrimidin-2-yl)amino)cyclohexyl)pyrrolidin-2-one [3452];
(1 s,4s)-1-methyl-4-((4-(methylamino)-5-(pyrazolo [1,5-a]pyrimidin-5-yl)-7H-pyrrolo [2,3-d]pyrimidin-2-yl)amino)cyclohexan-1-ol [3453];
(1 r,4r)-1-methyl-4-((4-(methylamino)-5-(pyrazolo [1,5-a]pyrimidin-5-yl)-7H-pyrrolo [2,3-d]pyrimidin-2-yl)amino)cyclohexan-1-ol [3454];
N²-((3 R,4 S)-3-fluoro-1-methylpiperidin-4-yl)-N⁴-methyl-5-(pyrazolo [1,5-a]pyrimidin-5-yl)-7H-pyrrolo [2,3-d]pyrimidine-2,4-diamine [3455];
N²-((3 S,4R)-3-fluoro-1-methylpiperidin-4-yl)-N⁴-methyl-5-(pyrazolo [1,5-a]pyrimidin-5-yl)-7H-pyrrolo [2,3-d]pyrimidine-2,4-diamine [3456];
N²-((3 S,4 S)-3-fluoro-1-methylpiperidin-4-yl)-N⁴-methyl-5-(pyrazolo [1,5-a]pyrimidin-5-yl)-7H-pyrrolo [2,3-d]pyrimidine-2,4-diamine [3457];
N²-((3 R,4R)-3-fluoro-1-methylpiperidin-4-yl)-N⁴-methyl-5-(pyrazolo [1,5-a]pyrimidin-5-yl)-7H-pyrrolo [2,3-d]pyrimidine-2,4-diamine [3458];
(2-(4-(methylamino)-5-(pyrazolo [1,5-a]pyrimidin-5-yl)-7H-pyrrolo [2,3-d]pyrimidin-2-yl)-2-azabicyclo [2 .2 .2]octan-4-yl)methanol [3459];
1-(7-((4-(methylamino)-5-(pyrazolo [1,5-a]pyrimidin-5-yl)-7H-pyrrolo [2,3-d]pyrimidin-2-yl)amino)-2-azaspiro [3 .5]nonan-2-yl)ethan-1-one [3460];
N⁴-methyl-5-(pyrazolo [1,5-a]pyrimidin-5-yl)-N²-(2-oxaspiro [3 .5]nonan-7-yl)-7H-pyrrolo [2,3-d]pyrimidine-2,4-diamine [3461];
(1 s,3 s)-3-((5-(cinnolin-6-yl)-7H-pyrrolo [2,3-d]pyrimidin-2-yl)amino)-N,N, 1-trimethylcyclobutane-1-carboxamide [3462];
(1 r,3 r)-3-((5-(cinnolin-6-yl)-7H-pyrrolo [2,3-d]pyrimidin-2-yl)amino)-N,N, 1-trimethylcyclobutane-1-carboxamide [3463];
((1 r,3 r)-3-((5-(cinnolin-6-yl)-7H-pyrrolo [2,3-d]pyrimidin-2-yl)amino)-1-methylcyclobutyl)(pyrrolidin-1-yl)methanone [3464];
((1 s,3 s)-3-((5-(cinnolin-6-yl)-7H-pyrrolo [2,3-d]pyrimidin-2-yl)amino)-1-methylcyclobutyl)(pyrrolidin-1-yl)methanone [3465];
N-((1 r,3 r)-3-((5-(cinnolin-6-yl)-7H-pyrrolo [2,3-d]pyrimidin-2-yl)amino)-1-methylcyclobutypacetamide [3466];
N-((1 s,3 s)-3-((5-(cinnolin-6-yl)-7H-pyrrolo [2,3-d]pyrimidin-2-yl)amino)-1-methylcyclobutypacetamide [3467];
N-((1s,3s)-3-45-(cinnolin-6-yl)-7H-pyrrolo [2,3-d] pyrimidin-2-yl)amino)-1-methylcyclobutyl)propionamide [3468];
N-((1r,30-3-45-(cinnolin-6-yl)-7H-pyrrolo [2,3-d] pyrimidin-2-yl)amino)-1-methylcyclobutyl)propionamide [3469];
1-((1r,30-3-45-(cinnolin-6-yl)-7H-pyrrolo [2,3-d]pyrimidin-2-yl)amino)-1-methylcyclobutyl)pyrrolidin-2-one [3470];
1-((1s,3s)-3-45-(cinnolin-6-yl)-7H-pyrrolo [2,3-d] pyrimidin-2-yl)amino)-1-methylcyclobutyl)pyrrolidin-2-one [3471];
(1s,3s)-3-45-(cinnolin-6-yl)-7H-pyrrolo [2,3-d]pyrimidin-2-yl)amino)-1-methylcyclobutan-1-ol [3472];
(1r,30-3-45-(cinnolin-6-yl)-7H-pyrrolo [2,3-d] pyrimidin-2-yl)amino)-1-methylcyclobutan-1-ol [3473];
(1s,4s)-4-45-(cinnolin-6-yl)-7H-pyrrolo [2,3-d]pyrimidin-2-yl)amino)-N,N-dimethylcyclohexane-1-carboxamide [3474];
((1s,4s)-4-45-(cinnolin-6-yl)-7H-pyrrolo [2,3-d] pyrimidin-2-yl)amino)cyclohexyl)(pyrrolidin-1-yl)methanone [3475];
N-((1s,4s)-4-45-(cinnolin-6-yl)-7H-pyrrolo [2,3-d] pyrimidin-2-yl)amino)cyclohexyl)acetamide [3476];
N-((1r,40-4-45-(cinnolin-6-yl)-7H-pyrrolo [2,3-d] pyrimidin-2-yl)amino)cyclohexyl)acetamide [3477];
1-((1s,4s)-4-45-(cinnolin-6-yl)-7H-pyrrolo [2,3-d] pyrimidin-2-yl)amino)cyclohexyl)pyrrolidin-2-one [3478];
2-(((1r,40-4-45-(cinnolin-6-yl)-7H-pyrrolo [2,3-d] pyrimidin-2-yl)amino)cyclohexyl)oxy)ethan-1-ol [3479];
2-(((1s,4s)-4-45-(cinnolin-6-yl)-7H-pyrrolo [2,3-d] pyrimidin-2-yl)amino)cyclohexyl)oxy)ethan-1-ol [3480];
N-((1r,40-4-45-(cinnolin-6-yl)-7H-pyrrolo [2,3-d] pyrimidin-2-yl)amino)-1-methylcyclohexyl)acetamide [3481];
(1s,4s)-4-45-(cinnolin-6-yl)-7H-pyrrolo [2,3-d]pyrimidin-2-yl)amino)-1-methylcyclohexan-1-ol [3482];
(1r,40-4-45-(cinnolin-6-yl)-7H-pyrrolo [2,3-d] pyrimidin-2-yl)amino)-1-methylcyclohexan-1-ol [3483];
(1s,4s)-4-45-(cinnolin-6-yl)-7H-pyrrolo [2,3-d]pyrimidin-2-yl)amino)-1-ethylcyclohexan-1-ol [3484];
1-(4-(cinnolin-6-yl)-7H-pyrrolo [2,3-d] pyrimidin-2-yl)amino)piperidin-1-yl)ethan-1-one [3485];
(S)-5-45-(cinnolin-6-yl)-7H-pyrrolo[2,3-d]pyrimidin-2-yl)amino)-1-methylpiperidin-2-one [3486];
(R)-5-45-(cinnolin-6-yl)-7H-pyrrolo[2,3-d]pyrimidin-2-yl)amino)-1-methylpiperidin-2-one [3487];
1-(2-(cinnolin-6-yl)-7H-pyrrolo [2,3-d] pyrimidin-2-yl)amino)-7-azaspiro [3 .5] nonan-7-yl)ethan-1-one [3488];
1-((3aR,5s,6aS)-5-45-(cinnolin-6-yl)-7H-pyrrolo[2,3-d]pyrimidin-2-yl)amino)hexahydrocyclopenta[c]pyrrol-2(1H)-yl)ethan-1-one [3489];
1-((3aR,5r,6aS)-5-45-(cinnolin-6-yl)-7H-pyrrolo[2,3-d]pyrimidin-2-yl)amino)hexahydrocyclopenta[c]pyrrol-2(1H)-yl)ethan-1-one [3490];
1474(5-(cinnolin-6-yl)-7H-pyrrolo [2,3-d] pyrimidin-2-yl)amino)-2-azaspiro [3.5] nonan-2-yl)ethan-1-one [3491];
5-(cinnolin-6-yl)-N-(2-oxaspiro [3 .5] nonan-7-yl)-7H-pyrrolo [2,3-d] pyrimidin-2-amine [3492];
5-(cinnolin-6-yl)-N-(1,4-dioxaspiro[4.5]decan-8-yl)-7H-pyrrolo[2,3-d]pyrimidin-2-amine [3493];
(1s,3s)-3-45-(cinnolin-6-yl)-4-methoxy-7H-pyrrolo[2,3-d]pyrimidin-2-yl)amino)-N,N,1-trimethylcyclobutane-1-carboxamide [3494];
(1r,30-3-45-(cinnolin-6-yl)-4-methoxy-7H-pyrrolo[2,3-d]pyrimidin-2-yl)amino)-N,N,1-trimethylcyclobutane-1-carboxamide [3495];
((1r,30-3-45-(cinnolin-6-yl)-4-methoxy-7H-pyrrolo[2,3-d]pyrimidin-2-yl)amino)-1-methylcyclobutyl)(pyrrolidin-1-yl)methanone [3496];
((1s,3s)-3-45-(cinnolin-6-yl)-4-methoxy-7H-pyrrolo[2,3-d]pyrimidin-2-yl)amino)-1-methylcyclobutyl)(pyrrolidin-1-yl)methanone [3497];
N-((1s,3s)-3-45-(cinnolin-6-yl)-4-methoxy-7H-pyrrolo[2,3-d]pyrimidin-2-yl)amino)-1-methylcyclobutypacetamide [3498];
N-((1r,3 r)-3-(cinnolin-6-yl)-4-methoxy-7H-pyrrolo [2,3-d] pyrimidin-2-yl)amino)-1-methylcyclobutypacetamide [3499];
N-((1s,3s)-3-45-(cinnolin-6-yl)-4-methoxy-7H-pyrrolo[2,3-d]pyrimidin-2-yl)amino)-1-methylcyclobutyl)propionamide [3500]; or a pharmaceutically acceptable salt thereof.

30. The compound of claim 1, wherein the compound of Formula I is selected from the group consisting of:
N-((1r,3 r)-3-(cinnolin-6-yl)-4-methoxy-7H-pyrrolo [2,3-d] pyrimidin-2-yl)amino)-1-methylcyclobutyl)propionamide [3501];
1-((1r,30-3-45-(cinnolin-6-yl)-4-methoxy-7H-pyrrolo [2,3-d] pyrimidin-2-yl)amino)-1-methylcyclobutyl)pyrrolidin-2-one [3502];
1-((1s,3s)-3-45-(cinnolin-6-yl)-4-methoxy-7H-pyrrolo [2,3-d]pyrimidin-2-yl)amino)-1-methylcyclobutyl)pyrrolidin-2-one [3503];
(1s,3s)-3-45-(cinnolin-6-yl)-4-methoxy-7H-pyrrolo [2,3-d]pyrimidin-2-yl)amino)-1-methylcyclobutan-1-ol [3504];
(1r,30-3-45-(cinnolin-6-yl)-4-methoxy-7H-pyrrolo [2,3-d] pyrimidin-2-yl)amino)-1-methylcyclobutan-1-ol [3505];
(1s,4s)-4-45-(cinnolin-6-yl)-4-methoxy-7H-pyrrolo [2,3-d]pyrimidin-2-yl)amino)-N,N-dimethylcyclohexane-1-carboxamide [3506];
((1s,4s)-4-45-(cinnolin-6-yl)-4-methoxy-7H-pyrrolo [2,3-d] pyrimidin-2-yl)amino)cyclohexyl)(pyrrolidin-1-yl)methanone [3507];
N-((1s,4s)-4-45-(cinnolin-6-yl)-4-methoxy-7H-pyrrolo [2,3-d]pyrimidin-2-yl)amino)cyclohexypacetamide [3508];
N-((1r,40-4-45-(cinnolin-6-yl)-4-methoxy-7H-pyrrolo [2,3-d]pyrimidin-2-yl)amino)cyclohexypacetamide [3509];
1-((1s,4s)-4-45-(cinnolin-6-yl)-4-methoxy-7H-pyrrolo [2,3-d]pyrimidin-2-yl)amino)cyclohexyl)pyrrolidin-2-one [3510];
2-(((1s,4s)-4-45-(cinnolin-6-yl)-4-methoxy-7H-pyrrolo [2,3-d] pyrimidin-2-yl)amino)cyclohexyl)oxy)ethan-1-ol [3511];
2-(((1r,40-4-45-(cinnolin-6-yl)-4-methoxy-7H-pyrrolo [2,3-d]pyrimidin-2-yl)amino)cyclohexyl)oxy)ethan-1-ol [3512];
N-((1r,40-4-45-(cinnolin-6-yl)-4-methoxy-7H-pyrrolo [2,3-d]pyrimidin-2-yl)amino)-1-methylcyclohexyl)acetamide [3513];
(1s,4s)-4-45-(cinnolin-6-yl)-4-methoxy-7H-pyrrolo [2,3-d]pyrimidin-2-yl)amino)-1-methylcyclohexan-1-ol [3514];
(1r,40-4-45-(cinnolin-6-yl)-4-methoxy-7H-pyrrolo [2,3-d] pyrimidin-2-yl)amino)-1-methylcyclohexan-1-ol [3515];
(1r,40-4-45-(cinnolin-6-yl)-4-(methoxy-d₃)-7H-pyrrolo [2,3-d] pyrimidin-2-yl)amino)-1-methylcyclohexan-1-ol [3516];
(1s,4s)-4-45-(cinnolin-6-yl)-4-(methoxy-d₃)-7H-pyrrolo [2,3-d]pyrimidin-2-yl)amino)-1-methylcyclohexan-1-ol [3517];
(1s,4s)-4-45-(cinnolin-6-yl)-4-methoxy-7H-pyrrolo [2,3-d]pyrimidin-2-yl)amino)-1-ethylcyclohexan-1-ol [3518];
1-(4-45-(cinnolin-6-yl)-4-methoxy-7H-pyrrolo [2,3-d]pyrimidin-2-yl)amino)piperidin-1-yl)ethan-1-one [3519];
(R)-5-45-(cinnolin-6-yl)-4-methoxy-7H-pyrrolo [2,3-d]pyrimidin-2-yl)amino)-1-methylpiperidin-2-one [3520];
(S)-5-45-(cinnolin-6-yl)-4-methoxy-7H-pyrrolo [2,3-d]pyrimidin-2-yl)amino)-1-methylpiperidin-2-one [3521];
1-(2-45-(cinnolin-6-yl)-4-methoxy-7H-pyrrolo [2,3-d]pyrimidin-2-yl)amino)-7-azaspiro [3 .5]nonan-7-yl)ethan-1-one [3522];
1-((3aR,5s,6aS)-5-45-(cinnolin-6-yl)-4-methoxy-7H-pyrrolo [2,3-d]pyrimidin-2-yl)amino)hexahydrocyclopenta[c]pyrrol-2(1H)-yl)ethan-1-one [3523];
1-((3aR,5r,6aS)-5-45-(cinnolin-6-yl)-4-methoxy-7H-pyrrolo [2,3-d] pyrimidin-2-yl)amino)hexahydrocyclopenta[c]pyrrol-2(1H)-yl)ethan-1-one [3524];
1-(7-45-(cinnolin-6-yl)-4-methoxy-7H-pyrrolo [2,3-d]pyrimidin-2-yl)amino)-2-azaspiro [3 .5]nonan-2-yl)ethan-1-one [3525];
5-(cinnolin-6-yl)-4-methoxy-N-(2-oxaspiro [3 .5]nonan-7-yl)-7H-pyrrolo [2,3-d]pyrimidin-2-amine [3526];
5-(cinnolin-6-yl)-4-methoxy-N-(1,4-dioxaspiro [4.5] de can-8-yl)-7H-pyrrolo [2,3-d]pyrimidin-2-amine [3527];
(1s,3s)-N,N,1-trimethyl-3-45-(quinoxalin-6-yl)-7H-pyrrolo [2,3-d] pyrimidin-2-yl)amino)cyclobutane-1-carboxamide [3528];
(1r,30-N,N,1-trimethyl-3-45-(quinoxalin-6-yl)-7H-pyrrolo [2,3-d] pyrimidin-2-yl)amino)cyclobutane-1-carboxamide [3529];
((1r,3r)-1-methyl-3-45-(quinoxalin-6-yl)-7H-pyrrolo [2,3-d]pyrimidin-2-yl)amino)cyclobutyl)(pyrrolidin-1-yl)methanone [3530];
((1s,3s)-1-methyl-3-45-(quinoxalin-6-yl)-7H-pyrrolo [2,3-d]pyrimidin-2-yl)amino)cyclobutyl)(pyrrolidin-1-yl)methanone [3531];
N-((1s,3s)-1-methyl-3-45-(quinoxalin-6-yl)-7H-pyrrolo [2,3-d] pyrimidin-2-yl)amino)cyclobutypacetamide [3532];
N-((1r,3r)-1-methyl-3-45-(quinoxalin-6-yl)-7H-pyrrolo [2,3-d] pyrimidin-2-yl)amino)cyclobutypacetamide [3533];
N-((1s,3s)-1-methyl-3-45-(quinoxalin-6-yl)-7H-pyrrolo [2,3-d] pyrimidin-2-yl)amino)cyclobutyl)propionamide [3534];
N-((1r,3r)-1-methyl-3-45-(quinoxalin-6-yl)-7H-pyrrolo [2,3-d] pyrimidin-2-yl)amino)cyclobutyl)propionamide [3535];
1-((1r,3r)-1-methyl-3-45-(quinoxalin-6-yl)-7H-pyrrolo [2,3-d]pyrimidin-2-yl)amino)cyclobutyppyrrolidin-2-one [3536];
1-((1s,3s)-1-methyl-3-45-(quinoxalin-6-yl)-7H-pyrrolo [2,3-d]pyrimidin-2-yl)amino)cyclobutyppyrrolidin-2-one [3537];
(1s,3s)-1-methyl-3-45-(quinoxalin-6-yl)-7H-pyrrolo [2,3-d]pyrimidin-2-yl)amino)cyclobutan-1-ol [3538];
(1r,3r)-1-methyl-3-45-(quinoxalin-6-yl)-7H-pyrrolo [2,3-d] pyrimidin-2-yl)amino)cyclobutan-1-ol [3539];
(1s,4s)-N,N-dimethyl-4-45-(quinoxalin-6-yl)-7H-pyrrolo [2,3-d] pyrimidin-2-yl)amino)cyclohexane-1-carboxamide [3540];
pyrrolidin-1-yl((1s,4s)-4-45-(quinoxalin-6-yl)-7H-pyrrolo [2,3-d] pyrimidin-2-yl)amino)cyclohexyl)methanone [3541];
N-((1s,4s)-4-45-(quinoxalin-6-yl)-7H-pyrrolo [2,3-d]pyrimidin-2-yl)amino)cyclohexypacetamide [3542];
N-((1r,40-4-45-(quinoxalin-6-yl)-7H-pyrrolo [2,3-d]pyrimidin-2-yl)amino)cyclohexypacetamide [3543];
1-((1s,4s)-4-((5-(quinoxalin-6-yl)-7H-pyrrolo[2,3-cl]pyrimidin-2-yl)amino)cyclohexyl)pyrrolidin-2-one [3544];
2-(((1r,40-4-45-(quinoxalin-6-yl)-7H-pyrrolo [2,3-d]pyrimidin-2-yl)amino)cyclohexyl)oxy)ethan-1-ol [3545];
N-((1r,4r)-1-methyl-4-45-(quinoxalin-6-yl)-7H-pyrrolo [2,3-d] pyrimidin-2-yl)amino)cyclohexyl)acetamide [3546];
(1r,4r)-1-methyl-4-45-(quinoxalin-6-yl)-7H-pyrrolo [2,3-d] pyrimidin-2-yl)amino)cyclohexan-1-ol [3547];
(1s,4s)-1-ethyl-4-45-(quinoxalin-6-yl)-7H-pyrrolo [2,3-d]pyrimidin-2-yl)amino)cyclohexan-1-ol [3548];
1-(4-45-(quinoxalin-6-yl)-7H-pyrrolo [2,3-d] pyrimidin-2-yl)amino)piperidin-1-yl)ethan-1-one [3549];
(S)-1-methyl-5-45-(quinoxalin-6-yl)-7H-pyrrolo [2,3-d]pyrimidin-2-yl)amino)piperidin-2-one [3550];
(R)-1-methyl-5-45-(quinoxalin-6-yl)-7H-pyrrolo [2,3-d]pyrimidin-2-yl)amino)piperidin-2-one [3551];
1-(2-45-(quinoxalin-6-yl)-7H-pyrrolo [2,3-d] pyrimidin-2-yl)amino)-7-azaspiro [3 .5]nonan-7-yl)ethan-1-one [3552];
1-((3aR,5r,6aS)-5-45-(quinoxalin-6-yl)-7H-pyrrolo [2,3-d]pyrimidin-2-yl)amino)hexahydrocyclopenta[c]pyrrol-2(1H)-yl)ethan-1-one [3553];
1-((3aR,5s,6aS)-5-45-(quinoxalin-6-yl)-7H-pyrrolo [2,3-d]pyrimidin-2-yl)amino)hexahydrocyclopenta[c]pyrrol-2(1H)-yl)ethan-1-one [3554];
1-(7-45-(quinoxalin-6-yl)-7H-pyrrolo [2,3-d] pyrimidin-2-yl)amino)-2-azaspiro [3 .5]nonan-2-yl)ethan-1-one [3555];
5-(quinoxalin-6-yl)-N-(2-oxaspiro [3 .5] nonan-7-yl)-7H-pyrrolo [2,3-d]pyrimidin-2-amine [3556];
5-(quinoxalin-6-yl)-N-(1,4-dioxaspiro [4.5] de can-8-yl)-7H-pyrrolo [2,3-d] pyrimidin-2-amine [3557];
(1r,30-3-44-methoxy-5-(quinoxalin-6-yl)-7H-pyrrolo [2,3-d]pyrimidin-2-yl)amino)-N,N,1-trimethylcyclobutane-1-carboxamide [3558];
(1s,3s)-3-44-methoxy-5-(quinoxalin-6-yl)-7H-pyrrolo [2,3-d]pyrimidin-2-yl)amino)-N,N,1-trimethylcyclobutane-1-carboxamide [3559];
((1s,3s)-3-44-methoxy-5-(quinoxalin-6-yl)-7H-pyrrolo [2,3-d]pyrimidin-2-yl)amino)-1-methylcyclobutyl)(pyrrolidin-1-yl)methanone [3560];
((1r,30-3-44-methoxy-5-(quinoxalin-6-yl)-7H-pyrrolo [2,3-d]pyrimidin-2-yl)amino)-1-methylcyclobutyl)(pyrrolidin-1-yl)methanone [3561];
N-((1s,3s)-3-44-methoxy-5-(quinoxalin-6-yl)-7H-pyrrolo [2,3-d]pyrimidin-2-yl)amino)-1-methylcyclobutypacetamide [3562];
N-((1r,30-3-44-methoxy-5-(quinoxalin-6-yl)-7H-pyrrolo [2,3-d]pyrimidin-2-yl)amino)-1-methylcyclobutypacetamide [3563];
N-((1s,3s)-3-44-methoxy-5-(quinoxalin-6-yl)-7H-pyrrolo [2,3-d]pyrimidin-2-yl)amino)-1-methylcyclobutyl)propionamide [3564];
N-((1r,30-3-44-methoxy-5-(quinoxalin-6-yl)-7H-pyrrolo [2,3-d]pyrimidin-2-yl)amino)-1-methylcyclobutyl)propionamide [3565];
1-((1r,30-3-44-methoxy-5-(quinoxalin-6-yl)-7H-pyrrolo [2,3-d]pyrimidin-2-yl)amino)-1-methylcyclobutyl)pyrrolidin-2-one [3566];
1-((1s,3s)-3-44-methoxy-5-(quinoxalin-6-yl)-7H-pyrrolo [2,3-d]pyrimidin-2-yl)amino)-1-methylcyclobutyl)pyrrolidin-2-one [3567];
(1s,3s)-3-44-methoxy-5-(quinoxalin-6-yl)-7H-pyrrolo [2,3-d]pyrimidin-2-yl)amino)-1-methylcyclobutan-1-ol [3568];
(1r,30-3-44-methoxy-5-(quinoxalin-6-yl)-7H-pyrrolo [2,3-d]pyrimidin-2-yl)amino)-1-methylcyclobutan-1-ol [3569];
(1s,4s)-4-44-methoxy-5-(quinoxalin-6-yl)-7H-pyrrolo [2,3-d] pyrimidin-2-yl)amino)-N,N-dimethylcyclohexane-1-carboxamide [3570];
((1s,4s)-4-44-methoxy-5-(quinoxalin-6-yl)-7H-pyrrolo [2,3-d]pyrimidin-2-yl)amino)cyclohexyl)(pyrrolidin-1-yl)methanone [3571];
N-((1s,4s)-4-44-methoxy-5-(quinoxalin-6-yl)-7H-pyrrolo [2,3-d]pyrimidin-2-yl)amino)cyclohexypacetamide [3572];
N-((1r,40-4-44-methoxy-5-(quinoxalin-6-yl)-7H-pyrrolo [2,3-d]pyrimidin-2-yl)amino)cyclohexypacetamide [3573];
1-((1s,4s)-4-44-methoxy-5-(quinoxalin-6-yl)-7H-pyrrolo [2,3-d]pyrimidin-2-yl)amino)cyclohexyl)pyrrolidin-2-one [3574];
4-methoxy-N-((1s,4s)-4-methoxycyclohexyl)-5-(quinoxalin-6-yl)-7H-pyrrolo [2,3-d]pyrimidin-2-amine [3575];
2-(((1s,4s)-4-44-methoxy-5-(quinoxalin-6-yl)-7H-pyrrolo [2,3-d] pyrimidin-2-yl)amino)cyclohexyl)oxy)ethan-1-ol [3576];
2-(((1r,40-4-44-methoxy-5-(quinoxalin-6-yl)-7H-pyrrolo [2,3-d]pyrimidin-2-yl)amino)cyclohexyl)oxy)ethan-1-ol [3577];
N-((1r,40-4-44-methoxy-5-(quinoxalin-6-yl)-7H-pyrrolo [2,3-d]pyrimidin-2-yl)amino)-1-methylcyclohexyl)acetamide [3578];
(1r,40-4-44-methoxy-5-(quinoxalin-6-yl)-7H-pyrrolo [2,3-d]pyrimidin-2-yl)amino)-1-methylcyclohexan-1-ol [3579];
(1r,40-4-44-(methoxy-d₃)-5-(quinoxalin-6-yl)-7H-pyrrolo [2,3-d]pyrimidin-2-yl)amino)-1-methylcyclohexan-1-ol [3580];
(1s,4s)-4-44-(methoxy-d₃)-5-(quinoxalin-6-yl)-7H-pyrrolo [2,3-d]pyrimidin-2-yl)amino)-1-methylcyclohexan-1-ol [3581];
(1s,4s)-1-ethyl-4-44-methoxy-5-(quinoxalin-6-yl)-7H-pyrrolo [2,3-d] pyrimidin-2-yl)amino)cyclohexan-1-01 [3582];
1-(4-44-methoxy-5-(quinoxalin-6-yl)-7H-pyrrolo [2,3-d]pyrimidin-2-yl)amino)piperidin-1-yl)ethan-1-one [3583];
(R)-5-((4-methoxy-5-(quinoxalin-6-yl)-7H-pyrrolo [2,3-d]pyrimidin-2-yl)amino)-1-methylpiperidin-2-one [3584];
(S)-5-44-methoxy-5-(quinoxalin-6-yl)-7H-pyrrolo [2,3-d]pyrimidin-2-yl)amino)-1-methylpiperidin-2-one [3585];
1-(2-44-methoxy-5-(quinoxalin-6-yl)-7H-pyrrolo [2,3-d]pyrimidin-2-yl)amino)-7-azaspiro [3 .5]nonan-7-yl)ethan-1-one [3586];
1-((3aR,5r,6aS)-5-44-methoxy-5-(quinoxalin-6-yl)-7H-pyrrolo [2,3-d]pyrimidin-2-yl)amino)hexahydrocyclopenta[c]pyrrol-2(1H)-yl)ethan-1-one [3587];
1-((3aR,5s,6aS)-5-44-methoxy-5-(quinoxalin-6-yl)-7H-pyrrolo [2,3-d]pyrimidin-2-yl)amino)hexahydrocyclopenta[c]pyrrol-2(1H)-yl)ethan-1-one [3588];
1-(7-44-methoxy-5-(quinoxalin-6-yl)-7H-pyrrolo [2,3-d]pyrimidin-2-yl)amino)-2-azaspiro [3 .5]nonan-2-yl)ethan-1-one [3589];
4-methoxy-5-(quinoxalin-6-yl)-N-(2-oxaspiro [3 .5] nonan-7-yl)-7H-pyrrolo [2,3-d]pyrimidin-2-amine [3590];
4-methoxy-5-(quinoxalin-6-yl)-N-(1,4-dioxaspiro [4.5] de can-8-yl)-7H-pyrrolo [2,3-d]pyrimidin-2-amine [3591];
(1s,4s)-N,N-dimethyl-4-44-(methylamino)-5-(1,5-naphthyridin-2-yl)-7H-pyrrolo [2,3-d]pyrimidin-2-yl)amino)cyclohexane-1-carboxamide [3592];
(1r,40-N,N-dimethyl-4-44-(methylamino)-5-(1,5-naphthyridin-2-yl)-7H-pyrrolo [2,3-d]pyrimidin-2-yl)amino)cyclohexane-1-carboxamide [3593];
((1s,4s)-4-((4-(methylamino)-5-(1,5-naphthyridin-2-yl)-7H-pyrrolo [2,3-d]pyrimidin-2-yl)amino)cyclohexyl)(pyrrolidin-1-yl)methanone [3594];
((1r,4r)-4-((4-(methylamino)-5-(1,5-naphthyridin-2-yl)-7H-pyrrolo [2,3-d]pyrimidin-2-yl)amino)cyclohexyl)(pyrrolidin-1-yl)methanone [3595];
N-((1s,4s)-4-((4-(methylamino)-5-(1,5-naphthyridin-2-yl)-7H-pyrrolo [2,3-d]pyrimidin-2-yl)amino)cyclohexypacetamide [3596];
N-((1r,4r)-4-((4-(methylamino)-5-(1,5-naphthyridin-2-yl)-7H-pyrrolo [2,3-d]pyrimidin-2-yl)amino)cyclohexypacetamide [3597];
1-((1s,4s)-4-((4-(methylamino)-5-(1,5-naphthyridin-2-yl)-7H-pyrrolo [2,3-d]pyrimidin-2-yl)amino)cyclohexyl)pyrrolidin-2-one [3598];
1-((1r,4r)-4-((4-(methylamino)-5-(1,5-naphthyridin-2-yl)-7H-pyrrolo [2,3-d]pyrimidin-2-yl)amino)cyclohexyl)pyrrolidin-2-one [3599];
N²-((1s,4s)-4-methoxycyclohexyl)-N⁴-methyl-5-(1,5-naphthyridin-2-yl)-7H-pyrrolo [2,3-d]pyrimidine-2,4-diamine [3600];
N-((1s,4s)-1-methyl-4-44-(methylamino)-5-(1,5-naphthyridin-2-yl)-7H-pyrrolo [2,3-d]pyrimidin-2-yl)amino)cyclohexyl)acetamide [3601];
N-((1r,40-1-methyl-4-44-(methylamino)-5-(1,5-naphthyridin-2-yl)-7H-pyrrolo [2,3-d]pyrimidin-2-yl)amino)cyclohexyl)acetamide [3602];
1-((1r,40-1-methyl-4-44-(methylamino)-5-(1,5-naphthyridin-2-yl)-7H-pyrrolo [2,3-d]pyrimidin-2-yl)amino)cyclohexyl)pyrrolidin-2-one [3603];
1-((1 s,4 s)-1-methyl-4-((4-(methylamino)-5-(1,5-naphthyridin-2-yl)-7H-pyrrolo [2,3-d]pyrimidin-2-yl)amino)cyclohexyl)pyrrolidin-2-one [3604];
N⁴-methyl-5-(1,5-naphthyridin-2-yl)-N²-(2-oxaspiro [3.5 ]nonan-7-yl)-7H-pyrrolo [2,3-d]pyrimidine-2,4-diamine [3605];
5-(imidazo [1,2-a] pyridin-6-yl)-N-44 s,7 s)-1-oxaspiro [3 .5]nonan-7-yl)-7H-pyrrolo [2,3-d]pyrimidin-2-amine [3606];
5-(imidazo [1,2-a] pyridin-6-yl)-N-((4r,7r) -1-oxaspiro [3 .5]nonan-7-yl)-7H-pyrrolo [2,3-d]pyrimidin-2-amine [3607];
5-(imidazo [1,2-a] pyridin-6-yl)-4-methoxy-N-44 s,7 s)-1-oxaspiro [3 . 5] nonan-7-yl)-7H-pyrrolo [2,3-d]pyrimidin-2-amine [3608];
5-(imidazo [1,2-a] pyridin-6-yl)-4-methoxy-N-44r,70-1-oxaspiro [3 .5 ]nonan-7-yl)-7H-pyrrolo [2,3-d]pyrimidin-2-amine [3609];
5-(8-fluoroimidazo [1,2-a]pyridin-6-yl)-N-44 s,7 s)-1-oxaspiro [3 . 5 ]nonan-7-yl)-7H-pyrrolo [2,3-d]pyrimidin-2-amine [3610];
5-(8-fluoroimidazo [1,2-a]pyridin-6-yl)-N-44r,7r) -1-oxaspiro [3 . 5 ]nonan-7-yl)-7H-pyrrolo [2,3-d]pyrimidin-2-amine [3611];
5-(8-fluoroimidazo [1,2-a]pyridin-6-yl)-4-methoxy-N-44 s,7 s)-1-oxaspiro [3 .5]nonan-7-yl)-7H-pyrrolo [2,3-d]pyrimidin-2-amine [3612];
5-(8-fluoroimidazo [1,2-a]pyridin-6-yl)-4-methoxy-N-44r,7r) -1-oxaspiro [3 .5 ]nonan-7-yl)-7H-pyrrolo [2,3-d]pyrimidin-2-amine [3613];
5-(3-fluoroimidazo [1,2-a]pyridin-6-yl)-N-44 s,7 s)-1-oxaspiro [3 . 5 ]nonan-7-yl)-7H-pyrrolo [2,3-d]pyrimidin-2-amine [3614];
5-(3-fluoroimidazo [1,2-a]pyridin-6-yl)-N-44r,7r) -1-oxaspiro [3 . 5 ]nonan-7-yl)-7H-pyrrolo [2,3-d]pyrimidin-2-amine [3615];
5-(3-fluoroimidazo [1,2-a]pyridin-6-yl)-4-methoxy-N-44r,7r) -1-oxaspiro [3 .5 ]nonan-7-yl)-7H-pyrrolo [2,3-d]pyrimidin-2-amine [3616];
5-(3-fluoroimidazo [1,2-a]pyridin-6-yl)-4-methoxy-N-44 s,7 s)-1-oxaspiro [3 .5]nonan-7-yl)-7H-pyrrolo [2,3-d]pyrimidin-2-amine [3617];
5-(imidazo [1,2-a] pyrimidin-6-yl)-N-44 s,7 s)-1-oxaspiro [3 .5 ]nonan-7-yl)-7H-pyrrolo [2,3-d]pyrimidin-2-amine [3618];
5-(imidazo [1,2-a] pyrimidin-6-yl)-N-44r,70-1-oxaspiro [3 . 5 ]nonan-7-yl)-7H-pyrrolo [2,3-d]pyrimidin-2-amine [3619];
5-(imidazo [1,2-a] pyrimidin-6-yl)-4-methoxy-N-44r,70-1-oxaspiro [3 . 5 ]nonan-7-yl)-7H-pyrrolo [2,3-d]pyrimidin-2-amine [3620];
5-(imidazo[1,2-a]pyrimidin-6-yl)-4-methoxy-N-44s,7s)-1-oxaspiro[3.5]nonan-7-yl)-7H-pyrrolo[2,3-d]pyrimidin-2-amine [3621];
5-(1-(2,2-difluoroethyl)-2-methyl-1H-imidazo[4,5-b]pyridin-6-yl)-N-44s,7s)-1-oxaspiro[3.5]nonan-7-yl)-7H-pyrrolo[2,3-d]pyrimidin-2-amine [3622];
5-(1-(2,2-difluoroethyl)-2-methyl-1H-imidazo[4,5-b]pyridin-6-yl)-N-44r,70-1-oxaspiro[3.5]nonan-7-yl)-7H-pyrrolo[2,3-d]pyrimidin-2-amine [3623];
5-(1-(2,2-difluoroethyl)-2-methyl-1H-imidazo[4,5-b]pyridin-6-yl)-4-methoxy-N-44r,70-1-oxaspiro[3.5]nonan-7-yl)-7H-pyrrolo[2,3-d]pyrimidin-2-amine [3624];
5-(1-(2,2-difluoroethyl)-2-methyl-1H-imidazo[4,5-b]pyridin-6-yl)-4-methoxy-N-44s,7s)-1-oxaspiro[3.5]nonan-7-yl)-7H-pyrrolo[2,3-d]pyrimidin-2-amine [3625];
(1s,4s)-4-45-(3-(2,2-difluoroethyl)-2-methyl-3H-imidazo[4,5-b]pyridin-5-yl)-4-(methylamino)-7H-pyrrolo[2,3-d]pyrimidin-2-yl)amino)-1-ethylcyclohexan-1-ol [3626];
5-(3-(2,2-difluoroethyl)-2-methyl-3H-imidazo[4,5-b]pyridin-5-yl)-N²-43S,4R)-3-fluoro-1-(oxetan-3-yl)piperidin-4-yl)-N⁴-methyl-7H-pyrrolo[2,3-d]pyrimidine-2,4-diamine [3627];
5-(3-(2,2-difluoroethyl)-2-methyl-3H-imidazo[4,5-b]pyridin-5-yl)-N²-43R,4S)-3-fluoro-1-(oxetan-3-yl)piperidin-4-yl)-N⁴-methyl-7H-pyrrolo[2,3-d]pyrimidine-2,4-diamine [3628];
5-(3-(2,2-difluoroethyl)-2-methyl-3H-imidazo[4,5-b]pyridin-5-yl)-N⁴-methyl-N²-44s,7s)-1-oxaspiro[3.5]nonan-7-yl)-7H-pyrrolo[2,3-d]pyrimidine-2,4-diamine [3629];
5-(3-(2,2-difluoroethyl)-2-methyl-3H-imidazo[4,5-b]pyridin-5-yl)-N⁴-methyl-N²-44r,70-1-oxaspiro[3.5]nonan-7-yl)-7H-pyrrolo[2,3-d]pyrimidine-2,4-diamine [3630];
5-([1,2,4]triazolo [4,3-a]pyridin-6-yl)-N-44s,7s)-1-oxaspiro [3 .5]nonan-7-yl)-7H-pyrrolo [2,3-d]pyrimidin-2-amine [3631];
5-([1,2,4]triazolo [4,3-a]pyridin-6-yl)-N-44r,70-1-oxaspiro [3 .5]nonan-7-yl)-7H-pyrrolo [2,3-d]pyrimidin-2-amine [3632];
5-([1,2,4]triazolo[4,3-a]pyridin-6-yl)-4-methoxy-N-44s,7s)-1-oxaspiro[3.5]nonan-7-yl)-7H-pyrrolo[2,3-d]pyrimidin-2-amine [3633];
4-methoxy-5-(3-methyl-[1,2,4]triazolo [4,3-a]pyridin-6-yl)-N-44s,7s)-1-oxaspiro [3.5]nonan-7-yl)-7H-pyrrolo[2,3-d]pyrimidin-2-amine [3634];
5-(3-methyl-[1,2,4]triazolo[4,3-a]pyridin-6-yl)-N-44s,7s)-1-oxaspiro [3 .5]nonan-7-yl)-7H-pyrrolo[2,3-d]pyrimidin-2-amine [3635];
5-(3-methyl-[1,2,4]triazolo[4,3-a]pyridin-6-yl)-N-44r,70-1-oxaspiro[3.5]nonan-7-yl)-7H-pyrrolo[2,3-d]pyrimidin-2-amine [3636];
5-([1,2,4]triazolo [4,3-a]pyridin-6-yl)-4-methoxy-N-44r,70-1-oxaspiro [3 .5]nonan-7-yl)-7H-pyrrolo[2,3-d]pyrimidin-2-amine [3637];
4-methoxy-5-(3-methyl-[1,2,4]triazolo[4,3-a]pyridin-6-yl)-N-44r,70-1-oxaspiro[3.5]nonan-7-yl)-7H-pyrrolo[2,3-d]pyrimidin-2-amine [3638];
5-([1,2,4]triazolo[1,5-a]pyridin-6-yl)-N-44s,7s)-1-oxaspiro[3.5]nonan-7-yl)-7H-pyrrolo[2,3-d]pyrimidin-2-amine [3639];
5-([1,2,4]triazolo[1,5-a]pyridin-6-yl)-N-44r,70-1-oxaspiro[3.5]nonan-7-yl)-7H-pyrrolo[2,3-d]pyrimidin-2-amine [3640];
5-([1,2,4]triazolo[1,5-a]pyridin-6-yl)-4-methoxy-N-44s,7s)-1-oxaspiro[3.5]nonan-7-yl)-7H-pyrrolo[2,3-d]pyrimidin-2-amine P6411;
5-([1,2,4]triazolo[1,5-a]pyridin-6-yl)-4-methoxy-N-44r,70-1-oxaspiro[3.5]nonan-7-yl)-7H-pyrrolo[2,3-d]pyrimidin-2-amine [3642];
(1s,4s)-1-methyl-4-((5-(1-methyl-1H-benzo[d][1,2,3]triazol-6-yl)-7H-pyrrolo[2,3-d]pyrimidin-2-yl)amino)cyclohexan-1-ol [3643];
5-(1-methyl-1H-benzo[d][1,2,3]triazol-6-yl)-N-44s,7s)-1-oxaspiro[3.5]nonan-7-yl)-7H-pyrrolo[2,3-d]pyrimidin-2-amine [3644];
5-(1-methyl-1H-benzo[d][1,2,3]triazol-6-yl)-N-44r,70-1-oxaspiro[3.5]nonan-7-yl)-7H-pyrrolo[2,3-d]pyrimidin-2-amine [3645];
4-methoxy-5-(1-methyl-1H-benzo[d][1,2,3]triazol-6-yl)-N-44r,70-1-oxaspiro[3.5]nonan-7-yl)-7H-pyrrolo[2,3-d]pyrimidin-2-amine [3646];
4-methoxy-5-(1-methyl-1H-benzo[d][1,2,3]triazol-6-yl)-N-44s,7s)-1-oxaspiro[3.5]nonan-7-yl)-7H-pyrrolo[2,3-d]pyrimidin-2-amine [3647];
5-(1-(2,2-difluoroethyl)-1H-benzo[d][1,2,3]triazol-6-yl)-N-44r,70-1-oxaspiro[3.5]nonan-7-yl)-7H-pyrrolo[2,3-d]pyrimidin-2-amine [3648];
5-(1-(2,2-difluoroethyl)-1H-benzo[d][1,2,3]triazol-6-yl)-N-44s,7s)-1-oxaspiro[3.5]nonan-7-yl)-7H-pyrrolo[2,3-d]pyrimidin-2-amine [3649];
5-(1-(2,2-difluoroethyl)-1H-benzo[d][1,2,3]triazol-6-yl)-4-methoxy-N-44r,70-1-oxaspiro[3.5]nonan-7-yl)-7H-pyrrolo[2,3-d]pyrimidin-2-amine [3650];
5-(1-(2,2-difluoroethyl)-1H-benzo[d][1,2,3]triazol-6-yl)-4-methoxy-N-44s,7s)-1-oxaspiro[3.5]nonan-7-yl)-7H-pyrrolo[2,3-d]pyrimidin-2-amine P6511;
(1s,4s)-1-ethyl-4-((5-(imidazo[1,2-b]pyridazin-6-yl)-4-(methylamino)-7H-pyrrolo[2,3-d]pyrimidin-2-yl)amino)cyclohexan-1-ol [3652];
N²-43S,4R)-3-fluoro-1-(oxetan-3-yl)piperidin-4-yl)-5-(imidazo[1,2-b]pyridazin-6-yl)-N⁴-methyl-7H-pyrrolo[2,3-d]pyrimidine-2,4-diamine [3653];
N²-43R,4S)-3-fluoro-1-(oxetan-3-yl)piperidin-4-yl)-5-(imidazo[1,2-b]pyridazin-6-yl)-N⁴-methyl-7H-pyrrolo[2,3-d]pyrimidine-2,4-diamine [3654];
5-(imidazo [1,2-b]pyridazin-6-yl)-N⁴-methyl-N²-44s,7s)-1-oxaspiro [3 .5]nonan-7-yl)-7H-pyrrolo [2,3-d]pyrimidine-2,4-diamine [3655];
5-(imidazo [1,2-b]pyridazin-6-yl)-N⁴-methyl-N²-44r,70-1-oxaspiro [3.5]nonan-7-yl)-7H-pyrrolo [2,3-d]pyrimidine-2,4-diamine [3656];
5-(pyrazolo [1,5-a]pyridin-5-yl)-N-44s,7s)-1-oxaspiro [3 .5] nonan-7-yl)-7H-pyrrolo [2,3-d]pyrimidin-2-amine [3657];
5-(pyrazolo [1,5-a]pyridin-5-yl)-N-44r,70-1-oxaspiro [3 .5]nonan-7-yl)-7H-pyrrolo [2,3-d]pyrimidin-2-amine [3658];
4-methoxy-5-(pyrazolo [1,5-a]pyridin-5-yl)-N-44s,7s)-1-oxaspiro [3 .5]nonan-7-yl)-7H-pyrrolo [2,3-d]pyrimidin-2-amine [3659];
4-methoxy-5-(pyrazolo [1,5-a]pyridin-5-yl)-N-44r,70-1-oxaspiro [3 .5]nonan-7-yl)-7H-pyrrolo [2,3-d]pyrimidin-2-amine [3660];
5-(2-(((4s,7s)-1-oxaspiro [3.5]nonan-7-yl)amino)-7H-pyrrolo [2,3-d]pyrimidin-5-yl)-N-methylpyrazolo [1,5-a]pyridine-3-carboxamide [3661];
5-(2-(((4r,7r)-1-oxaspiro [3 .5] nonan-7-yl)amino)-7H-pyrrolo [2,3-d] pyrimidin-5-yl)-N-methylpyrazolo [1,5-a]pyridine-3-carboxamide [3662];
5-(2-(((4s,7s)-1-oxaspiro [3.5]nonan-7-yl)amino)-4-methoxy-7H-pyrrolo [2,3-d] pyrimidin-5-yl)-N-methylpyrazolo [1,5-a] pyridine-3-carboxamide [3663];
5-(2-(((4r,7r)-1-oxaspiro [3 .5] nonan-7-yl)amino)-4-methoxy-7H-pyrrolo [2,3-d]pyrimidin-5-yl)-N-methylpyrazolo [1,5-a]pyridine-3-carboxamide [3664];
5-([1,2,4]triazolo [1,5-a]pyridin-7-yl)-N-44s,7s)-1-oxaspiro [3 .5]nonan-7-yl)-7H-pyrrolo [2,3-d]pyrimidin-2-amine [3665];
5-([1,2,4]triazolo [1,5-a]pyridin-7-yl)-N-44r,70-1-oxaspiro [3 .5]nonan-7-yl)-7H-pyrrolo [2,3-d]pyrimidin-2-amine [3666];
5-([1,2,4]triazolo [1,5-a]pyridin-7-yl)-4-methoxy-N-44s,7s)-1-oxaspiro [3 .5]nonan-7-yl)-7H-pyrrolo [2,3-d]pyrimidin-2-amine [3667];
5-([1,2,4]triazolo [1,5-a]pyridin-7-yl)-4-methoxy-N-44r,70-1-oxaspiro [3 .5]nonan-7-yl)-7H-pyrrolo [2,3-d]pyrimidin-2-amine [3668];
(1s,4s)-1-ethyl-4-44-(methylamino)-5-(pyrazolo [1,5-a] pyrimidin-5-yl)-7H-pyrrolo [2,3-d]pyrimidin-2-yl)amino)cyclohexan-1-ol [3669];
N²-((3 S,4R)-3-fluoro-1-(oxetan-3-yl)piperidin-4-yl)-N⁴-methyl-5-(pyrazolo [1,5-a]pyrimidin-5-yl)-7H-pyrrolo [2,3-d] pyrimidine -2,4-diamine [3670];
N²-43R,4S)-3-fluoro-1-(oxetan-3-yl)piperidin-4-yl)-N⁴-methyl-5-(pyrazolo [1,5-a]pyrimidin-5-yl)-7H-pyrrolo [2,3-d] pyrimidine -2,4-diamine [3671];
N⁴-methyl-5-(pyrazolo [1,5-a] pyrimidin-5-yl)-N²-445,75)-1-oxaspiro [3 .5] nonan-7-yl)-7H-pyrrolo [2,3-d]pyrimidine-2,4-diamine [3672];
N⁴-methyl-5-(pyrazolo[1,5-a]pyrimidin-5-yl)-N²-44r,70-1-oxaspiro[3.5]nonan-7-yl)-7H-pyrrolo[2,3-d]pyrimidine-2,4-diamine [3673];
5-(quinoxalin-6-yl)-N-((4r,7r)-1-oxaspiro [3 .5] nonan-7-yl)-7H-pyrrolo [2,3-d] pyrimidin-2-amine [3674];
5-(quinoxalin-6-yl)-N-((4s,7s)-1-oxaspiro [3 .5] nonan-7-yl)-7H-pyrrolo [2,3-d] pyrimidin-2-amine [3675];
4-methoxy-5-(quinoxalin-6-yl)-N-((4s,7s)-1-oxaspiro [3.5]nonan-7-yl)-7H-pyrrolo [2,3-d]pyrimidin-2-amine [3676];
4-methoxy-5-(quinoxalin-6-yl)-N-((4r,7r)-1-oxaspiro [3.5]nonan-7-yl)-7H-pyrrolo [2,3-d]pyrimidin-2-amine [3677]; or a pharmaceutically acceptable salt thereof.

31. The compound of claim 12, wherein the compound of Formula Ic is selected from the group consisting of:
2-(7H-pyrrolo [2,3-d] pyrimidin-5-yl)thieno [3,2-c] pyridine [55];
2-(7H-pyrrolo [2,3-d] pyrimidin-5-yl)thiazolo [5,4-b] pyridine [98];
5-(imidazo [1,2-a] pyridin-6-yl)-7H-pyrrolo [2,3-d] pyrimidine [99];
5-(3-fluoroimidazo [1,2-a] pyridin-6-yl)-7H-pyrrolo [2,3-d] pyrimidine [142];
5-(8-fluoroimidazo[1,2-a]pyridin-6-yl)-7H-pyrrolo[2,3-d]pyrimidine [185];
5-(8-fluoro -2-methylimidazo [1,2-a] pyridin-6-yl)-7H-pyrrolo [2,3-d] pyrimidine [230];
5-(8-fluoro -3-methylimidazo [1,2-a] pyridin-6-yl)-7H-pyrrolo [2,3-d] pyrimidine [273];
5-(3-(difluoromethyl)imidazo [1,2-a] pyridin-6-yl)-7H-pyrrolo [2,3-d] pyrimidine [316];
(8-fluoro-6-(7H-pyrrolo[2,3-d]pyrimidin-5-yl)imidazo[1,2-a]pyridin-3-yl)methanol [360];
N-(2,2-difluoroethyl)-6-(7H-pyrrolo[2,3-d]pyrimidin-5-yl)imidazo[1,2-a]pyridine-3-carboxamide [404];
5-(imidazo[1,2-a]pyrimidin-6-yl)-7H-pyrrolo[2,3-d]pyrimidine [447];
5-(imidazo[1,2-a]pyrazin-6-yl)-7H-pyrrolo[2,3-d]pyrimidine [491];
5-(4-fluoro-1-isopropyl-2-methyl-1H-benzo[d]imidazol-6-yl)-7H-pyrrolo[2,3-d]pyrimidine [495];
5-(4-fluoro-2-methyl-1-(1-methylpiperidin-4-yl)-1H-benzo [d]imidazol-6-yl)-7H-pyrrolo [2,3-d]pyrimidine [541];
1-isopropyl-2-methyl-6-(7H-pyrrolo[2,3-d]pyrimidin-5-yl)-1H-imidazo[4,5-b]pyridine [584];
1-isopropyl-2-methyl-6-(7H-pyrrolo[2,3-d]pyrimidin-5-yl)-1H-imidazo[4,5-c]pyridine [627];
3-(2,2-difluoroethyl)-2-methyl-5-(7H-pyrrolo[2,3-d]pyrimidin-5-yl)-3H-imidazo[4,5-b]pyridine [630];
3-i sopropyl-2-methyl-5-(7H-pyrrolo [2,3-d]pyrimidin-5-yl)-3H-imidazo [4,5-b] pyridine [673];
6-(7H-pyrrolo [2,3-d]pyrimidin-5-yl)41,2,41triazolo [1,5-a]pyridine [716];
8-fluoro -6-(7H-pyrrolo [2,3-d]pyrimidin-5-yl)-[1,2,4]triazolo [1,5-a]pyridine [761];
1-methyl-6-(7H-pyrrolo [2,3-d] pyrimidin-5-yl)-1H-benzo [d] [1,2,3]triazole [809];
1-ethyl-6-(7H-pyrrolo [2,3-d]pyrimidin-5-yl)-1H-benzo [d] [1,2,3]triazole [852];
5-(imidazo [1,2-b]pyridazin-6-yl)-7H-pyrrolo [2,3-d]pyrimidine [896];
5-(3-chloroimidazo [1,2-b]pyridazin-6-yl)-7H-pyrrolo [2,3-d]pyrimidine [939];
5-(2-methylimidazo [1,2-b]pyridazin-6-yl)-7H-pyrrolo [2,3-d]pyrimidine [982];
5-(3-methylimidazo [1,2-b]pyridazin-6-yl)-7H-pyrrolo [2,3-d]pyrimidine [1025];
5-(3-(difluoromethyl)imidazo [1,2-b]pyridazin-6-yl)-7H-pyrrolo [2,3-d] pyrimidine [1068];
5-(pyrazolo [1,5-a]pyridin-5-yl)-7H-pyrrolo [2,3-d] pyrimidine [1111];
5-(3-(1-methyl-1H-pyrazol-4-yl)pyrazolo [1,5-a]pyridin-5-yl)-7H-pyrrolo [2,3-d]pyrimidine [1158];
(5-(7H-pyrrolo [2,3-d]pyrimidin-5-yl)pyrazolo [1,5-a]pyridin-3-yl)(pipe ridin-1-yl)methanone [1201];
N-(2,2-difluoroethyl)-5-(7H-pyrrolo [2,3-d] pyrimidin-5-yl)pyrazolo [1,5-a]pyridine-3-carboxamide [1246];
N-(2-fluoro-2-methylpropyl)-5-(7H-pyrrolo [2,3-d] pyrimidin-5-yl)pyrazolo [1,5-a]pyridine-3-carboxamide [1289];
(R)-5-(7H-pyrrolo [2,3-d]pyrimidin-5-yl)-N-(1,1,1-trifluoropropan-2-yl)pyrazolo [1,5-a]pyridine-3-carboxamide [1332];
N-(3 ,3-difluorocyclobutyl)-5-(7H-pyrrolo [2,3-d]pyrimidin-5-yl)pyrazolo [1,5-a] pyridine-3-carboxamide [1375];
N-((3,3-difluorocyclobutyl)methyl)-5-(7H-pyrrolo [2,3-d]pyrimidin-5-yl)pyrazolo [1,5-a]pyridine-3-carboxamide [1419];
N-(trans-4-methoxycyclohexyl)-5-(7H-pyrrolo [2,3-d] pyrimidin-5-yl)pyrazolo [1,5-a]pyridine-3-carboxamide [1462];
N-(1-methylpiperidin-4-yl)-5-(7H-pyrrolo [2,3-d]pyrimidin-5-yl)pyrazolo [1,5-a]pyridine-3-carboxamide [1505];
5-(7H-pyrrolo [2,3-d]pyrimidin-5-yl)-N-(tetrahydro-2H-pyran-4-yl)pyrazolo [1,5-a]pyridine-3-carboxamide [1548];
N-(pyridin-3-yl)-5-(7H-pyrrolo [2,3-d] pyrimidin-5-yl)pyrazolo [1,5-a]pyridine-3-carboxamide [1592];
5-(pyrazolo [1,5-a]pyrimidin-5-yl)-7H-pyrrolo [2,3-d]pyrimidine [1635];
5-(3-cyclopropylpyrazolo [1,5-a] pyrimidin-5-yl)-7H-pyrrolo [2,3-d] pyrimidine [1682];
8-(7H-pyrrolo [2,3-d] pyrimidin -5-yl)-3 ,4-dihydrobenzo [f] [1 ,4] oxazepin -5 (2H)-one [1725];
2,2-dimethyl-7-(7H-pyrrolo [2,3-d]pyrimidin-5-yl)chroman-4-one [1769];
4,4-dimethyl-6-(7H-pyrrolo [2,3-d]pyrimidin-5-yl)-3,4-dihydroisoquinolin-1(2H)-one [1812];
6-(7H-pyrrolo [2,3-d] pyrimidin -5-yl)quinoline [1855];
6-(7H-pyrrolo [2,3-d] pyrimidin -5-yl)quinoxaline [1900];
6-(7H-pyrrolo [2,3-d] pyrimidin -5-yl)quinazoline [1943];
2-((l-methylpiperidin-4-yl)oxy)-7-(7H-pyrrolo [2,3-d]pyrimidin-5-yl)quinoxaline [1988]; or a pharmaceutically acceptable salt thereof.

32. The compound of claim 15, wherein the compound of Formula I is selected from the group consisting of:
N-(2-fluoro-2-methylpropyl)-5-(4-methoxyphenyl)-7H-pyrrolo [2,3-d]pyrimidin-2-amine [1];
5-(2,4-dimethoxyphenyl)-N-(1-(methylsulfonyl)piperidin-4-yl)-7H-pyrrolo [2,3-d]pyrimidin-2-amine [2];
5-(2-((2-fluoro-2-methylpropyl)amino)-7H-pyrrolo [2,3-d]pyrimidin-5-yl)-1-methylpyridin-2(1H)-one [3];
5-(2-((4,4-difluorocyclohexyl)amino)-7H-pyrrolo [2,3-d]pyrimidin-5-yl)-1-methylpyridin-2(1H)-one [4];
5-(6-(difluoromethyl)pyridin-3-yl)-N-(2-fluoro-2-methylpropyl)-7H-pyrrolo [2,3-d]pyrimidin-2-amine [5];
N-(2-fluoro-2-methylpropyl)-5-(6-methoxypyridin-3-yl)-7H-pyrrolo [2,3-d]pyrimidin-2-amine [6];
5-(6-(difluoromethoxy)pyridin -3-yl)-N-(2-fluoro -2-methylpropyl) -7H-pyrrolo [2,3-d] pyrimidin-2-amine [7];
5-(6-methoxypyridin-3-yl)-N-(trans-4-morpholinocyclohexyl)-7H-pyrrolo [2,3-d]pyrimidin-4-amine [8];
N-(2-fluoro-2-methylpropyl)-5-(2-methoxypyrimidin-5-yl)-7H-pyrrolo[2,3-d]pyrimidin-2-amine [9]; or a pharmaceutically acceptable salt thereof.

33. A pharmaceutical composition comprising a therapeutically effective amount of a compound of claim 1, or a pharmaceutically acceptable salt thereof, and a pharmaceutically acceptable excipient.

34. A pharmaceutical composition comprising a therapeutically effective amount of a compound of claim 12, or a pharmaceutically acceptable salt thereof, and a pharmaceutically acceptable excipient.

35. A pharmaceutical composition comprising a therapeutically effective amount of a compound of claim 15, or a pharmaceutically acceptable salt thereof, and a pharmaceutically acceptable excipient.

36. A method of treating a disorder or disease in a patient, wherein the disorder or disease is selected from the group consisting of: a neurological disorder, diabetes, and cancer, the method comprising administering to the patient a therapeutically effective amount of a compound of claim 1, or a pharmaceutically acceptable salt.

37. A method of treating a disorder or disease in a patient, wherein the disorder or disease is selected from the group consisting of: a neurological disorder, diabetes, and cancer, the method comprising administering to the patient a therapeutically effective amount of a compound of claim 12, or a pharmaceutically acceptable salt.

38. A method of treating a disorder or disease in a patient, wherein the disorder or disease is selected from the group consisting of: a neurological disorder, diabetes, and cancer, the method comprising administering to the patient a therapeutically effective amount of a compound of claim 15, or a pharmaceutically acceptable salt.
